(19) United States
(12) Patent Application Publication
(10) Pub. No.: US 2023/0212610 A1
(43) Pub. Date: Jul. 6, 2023

(54) NUCLEIC ACID EXPRESSION USING SUBCUTANEOUS ADMINISTRATION

(71) Applicant: DNARx, San Francisco, CA (US)

(72) Inventors: Robert DEBS, San Francisco, CA (US); Chakkrapong HANDUMRONGKUL, Richmond, VA (US); Timothy HEATH, Madison, WI (US); Alice YE, San Francisco, CA (US); Ryan ICE, Richmond, CA (US); Marissa MACK, Richmond, VA (US)

(73) Assignee: DNARx, San Francisco, CA (US)

(21) Appl. No.: 18/068,351

(22) Filed: Dec. 19, 2022

Related U.S. Application Data
(60) Provisional application No. 63/291,727, filed on Dec. 20, 2021.

(51) Int. Cl. C12N 15/86; A61K 47/69

(52) U.S. Cl. CPC C12N 15/86; A61K 47/6911

(57) ABSTRACT

Provided herein are compositions, systems, kits, and methods for treating a subject, and/or a subject's pre-adipocytes and/or adipocytes, with a composition containing a nucleic acid sequence encoding a protein or other biologically active nucleic acid-encoded molecule (BANEM), or a vector containing the nucleic acid sequence, wherein the treating comprises: a) injecting the composition into one or more subcutaneous (SC) regions of the subject such that one or more protein, or other BANEM, is detectable in a blood, serum, or plasma sample from the subject; and/or b) injecting the composition into one or more SC regions of the subject such that in-vivo transfected pre-adipocytes and/or adipocytes (e.g., transfected cells of fat cell origin) are generated; and/or c) performing the following: i) contacting pre-adipocytes and/or adipocytes (e.g., cells of fat cell origin) from the subject ex-vivo with the composition such that ex-vivo transfected pre-adipocytes and/or adipocytes are generated, and ii) injecting the ex-vivo transfected pre-adipocytes and/or adipocytes into one or more SC regions of the subject.

### CROSS-REFERENCE TO RELATED APPLICATIONS

This application claims priority to U.S. Provisional Application No. 63/291,727, filed on Dec. 20, 2021, the entirely of which is incorporated herein by reference.

### REFERENCE TO SEQUENCE LISTING SUBMITTED ELECTRONICALLY

This application contains a computer readable Sequence Listing which has been submitted in XML file format via Patent Center, the entire content of which is incorporated by reference herein in its entirety. The Sequence Listing XML file submitted via Patent Center is entitled “14755-007-999_SEQ_LISTING.xml”, was created on Dec. 17, 2022, and is 3,985,876 bytes in size.

### FIELD

Provided herein are compositions, systems, kits, and methods for treating a subject, and/or a subject's pre-adipocytes and/or adipocytes, with a composition containing a nucleic acid sequence encoding a protein or other biologically active nucleic acid-encoded molecule (BANEM), or a vector containing the nucleic acid sequence, wherein the treating comprises: a) injecting the composition into one or more subcutaneous (SC) regions of the subject such that one or more protein, or other BANEM, is detectable in a blood, serum, or plasma sample from the subject; and/or b) injecting the composition into one or more SC regions of the subject such that in-vivo transfected pre-adipocytes and/or adipocytes (e.g., transfected cells of fat cell origin) are generated; and/or c) performing the following: i) contacting pre-adipocytes and/or adipocytes (e.g., cells of fat cell origin) from the subject ex-vivo with the composition such that ex-vivo transfected pre-adipocytes and/or adipocytes are generated, and ii) injecting the ex-vivo transfected pre-adipocytes and/or adipocytes into one or more SC regions of the subject.

### BACKGROUND

The simplest non-viral gene delivery system uses naked expression vector DNA. Direct injection of free DNA into certain tissues, particularly muscle, has been shown to produce high levels of gene expression, and the simplicity of this approach has led to its adoption in a number of clinical protocols. In particular, this approach has been applied to the gene therapy of cancer where the DNA can be injected either directly into the tumor or can be injected into muscle cells in order to express tumor antigens that might function as a cancer vaccine.

Although direct injection of plasmid DNA into muscle has been shown to lead to gene expression, the overall level of expression is much lower than with either viral or liposomal vectors. Systemic administration of naked DNA is also generally thought to be unsuitable for systemic administration due to the presence of serum nucleases. As a result, direct injection of plasmid DNA appears to be limited to only a few applications involving tissues that are easily accessible to direct injection such as skin and muscle cells.

### SUMMARY

Provided herein are compositions, systems, kits, and methods for treating a subject, and/or a subject's adipocytes, with a composition (e.g., liquid composition) containing a nucleic acid sequence encoding a protein or a biologically active nucleic acid molecule (BANEM), or a vector containing the nucleic acid sequence, wherein the treating comprises: a) injecting the composition into one or more subcutaneous

(SC) regions of the subject such that the at least one protein, or BANEM, is detectable in a blood, serum, or plasma sample from the subject; and/or b) injecting the composition into one or more subcutaneous (SC) regions of the subject such that in-vivo transfected pre-adipocytes and/or adipocytes (e.g., transfected cells of fat cell origin) are generated; and/or c) performing the following: i) contacting transfected cells of fat cell origin from the subject ex-vivo with the composition such that ex-vivo transfected pre-adipocytes and/or adipocytes are generated, and ii) injecting the ex-vivo transfected pre-adipocytes and/or adipocytes into one or more SC regions of the subject.

Also provided herein is a method comprising treating a subject, and/or said subject's subcutaneous pre-adipocytes and/or adipocytes, with a composition, wherein said composition comprises, or consists essentially of, a nucleic acid sequence, or a vector containing said nucleic acid sequence, wherein said nucleic acid sequence encodes at least one protein or at least one biologically active nucleic acid molecule, and

wherein said treating comprises at least one of the following:
a) injecting said composition into one or more subcutaneous regions of said subject such that said at least one protein, or said at least one biologically active nucleic acid molecule, is detectable in a blood, serum, or plasma sample from said subject; and/or
b) injecting said composition into one or more subcutaneous regions of said subject such that a plurality of in-vivo transfected pre-adipocytes and/or adipocytes are generated in said subcutaneous region; and/or
c) performing the following:
   i) contacting a plurality said pre-adipocytes and/or adipocytes from said subject ex-vivo with said composition such that a plurality of ex-vivo transfected pre-adipocytes and/or adipocytes are generated, and
   ii) injecting at least some of said plurality of ex-vivo transfected pre-adipocytes and/or adipocytes into one or more subcutaneous regions of said subject.

Also provided herein is a method for the prevention, management or treatment of a disease or condition in a subject. In some embodiments, the disease or condition is associated with lipid storage disorders, such as but are not limited to Fabry disease, Gaucher disease, multiple sulfatase deficiency, Farber's lipogranulomatosis, Niemann-Pick disease, Wolman disease. In some embodiments, the disease or condition associated with lysosomal storage disorders, such as but are not limited to Schindler disease, Fucosidosis, Pompe disease, and Galactosialidosis. In some embodiments, the disease or condition is associated with a genetic disorder. In some embodiments, the disease or condition is associated with infection by a pathogen.

Also provided herein is a system comprising:
a) a plurality of transfected and enlarged adipocytes or pre-adipocytes, wherein each of said plurality of transfected and enlarged adipocytes or pre-adipocytes comprises an exogenous nucleic acid sequence, or a vector containing said nucleic acid sequence, wherein said nucleic acid sequence encodes at least one protein or at least one biologically active nucleic acid molecule, and
b) a first container, wherein said plurality of transfected and enlarged adipocytes or pre-adipocytes are present in said first container.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 shows the results from EXAMPLE 1, which shows serum expression levels (of anti-Sars-CoV-2 antibody) in mice receiving subcutaneously injected plasmids without any expression aid. Some mice also received a neutral lipid injection.

FIG. 2 shows the results from EXAMPLE 1, which shows serum expression levels (of human GCSF (h-GCSF)) in mice receiving subcutaneously injected plasmids encoding h-GCSF without any expression aid. Some mice also received chloroquine or neutral liposome.

FIG. 3 shows the results from EXAMPLE 2, which shows serum expression levels in rats receiving subcutaneously injected plasmids, with or without hyaluronidase.

FIG. 4 shows the results from EXAMPLE 3, which shows serum expression levels in rats receiving subcutaneously injected plasmids, with and without various pre-treatments.

FIG. 5 shows the results from EXAMPLE 4, which shows serum expression levels in rats receiving subcutaneously injected plasmids, with and without, lipid Pre-Treatment.

FIG. 6 shows the results from EXAMPLE 2, which shows long-term follow up of serum expression levels in rats receiving subcutaneously injected plasmids.

FIG. 7 shows the results from EXAMPLE 3, which shows serum protein level in rats receiving subcutaneously injected plasmids over 50 days, with or without hyaluronidase or pre-treatment with rituximab to deplete B cells.

FIG. 8 shows the results from EXAMPLE 4, which shows the level of the anti-Cov2 monoclonal antibody (209K) in female Sprague-Dawley rats (about 300 gram) receiving subcutaneously injected plasmids over an observation window of 106 days.

FIG. 9 shows the results from EXAMPLE 5, which shows in vitro transfection of human derived preadipocyte cells with a plasmid encoding 209K (anti-Cov2 antibody) using expifectamine (Expi), Polyethylenimine (PEI) or Dotap (DP). Cells were transfected via a direct (D) or thin-film (TF) method and a sample was collected every three days and assayed via hIgG ELISA. Values are mean SEM (n=2).

FIG. 10 shows the results from EXAMPLE 6, which shows in vitro transfection of human derived preadipocyte at various stages of the differentiation process towards adipocyte. Each stage of the differentiation cycle, starting with undifferentiated preadipocytes up to mature adipocytes, was transfected with the 209K (anti-CoV2 antibody) encoding plasmid using expifectamine. The media was changed out every three days and assayed via hIgG ELISA. Values are mean SEM (n=2).

FIG. 11 shows the results from EXAMPLE 7, which shows in vitro transfection of human adipocytes with 209K (anti-CoV-2 antibody) encoding plasmid using expifectamine (Expi), Polyethylenimine (PEI) or Dotap (DP). Cells were transfected via a direct (D) or thin-film (TF) method with either 1 or 5 μg plasmid DNA. Samples were collected every two days and assayed via hIgG ELISA. Values are mean SEM (n=2).

FIG. 12 shows the results from EXAMPLE 8, which shows the absolute neutrophil count (ANC) in obese mice 15 days after subcutaneous injection of plasmids encoding hGH-hGCSF.

FIG. 13 shows the results from EXAMPLE 9, which shows the hGLA expression level over 119 days in mice receiving subcutaneous injection of plasmids encoding hGLA-1×L-hyFc in 1 mL solution.

FIG. 14 shows the results from EXAMPLE 10, which shows high and long-term expression of hGLA in mice by subcutaneous injection of plasmids encoding hGLA-1×L-hyFc in smaller volumes.

FIG. 15 shows the results from EXAMPLE 11, which shows no expression of Factor VIII or IX in mice receiving subcutaneous injection of the encoding plasmid.

FIG. 16 shows the results from EXAMPLE 12, which shows the expression of antibody against SARS-CoV-2 by inguinal injections of plasmids at different doses and concentrations.

FIG. 17 shows the lack of correlation between the expression level of SARS-CoV-2 antibody and the body weight of the mice. No clear correlation between expression level and mice weight is observed.

FIG. 18 shows the results from EXAMPLE 13, which shows the expression level of SARS-CoV-2 antibody in mice administrated with the plasmid by surgical incision into the fat pad.

FIG. 19 shows the results from EXAMPLE 14, which shows the expression level of SARS-CoV-2 antibody in mice previously injected with a different plasmid.

FIG. 20 shows the results from EXAMPLE 15, which shows the expression level of hGLA in mice at various plasmid concentrations and hyaluronidase conditions.

FIG. 21 shows the lack of correlation between hGLA expression level and the body weight of mice. No clear correlation is observed.

FIG. 22 shows the results from EXAMPLE 17, which shows the expression level of hGLA with or without pretreatment with dexamethasone or TGF-beta3.

FIG. 23A shows the vector map for the base vector which is 1206 base pairs in length.

FIG. 23B illustrates the nucleic acid sequence for the base vector (SEQ ID NO:10).

FIG. 24A shows the vector map for the GLA-1×L-hyFc-Amp vector which is 5596 base pairs in length.

FIG. 24B illustrates the nucleic acid sequence for the GLA-1×L-hyFc-Amp vector (SEQ ID NO:11).

FIG. 25A shows the vector map for the GLA-1×L-hyFc-BV2 vector which is 4146 base pairs in length.

FIG. 25B illustrates the nucleic acid sequence for the GLA-1×L-hyFc-BV2 vector (SEQ ID NO:12).

FIG. 26A shows the vector map for the aCoV2-209 (H-P2A-L)-BV3 vector which is 5533 base pairs in length.

FIG. 26B illustrates the nucleic acid sequence for the aCoV2-209 (H-P2A-L)-BV3 vector (SEQ ID NO:13).

FIG. 27A shows the vector map for the aCoV2-209 (H-L)-BV3 vector which is 7298 base pairs in length.

FIG. 27B illustrates the nucleic acid sequence for the aCoV2-209 (H-L)-BV3 vector (SEQ ID NO:14).

FIG. 28A shows the vector map for the GHFc-GLA1×LhyFc-BV2 vector which is 6396 base pairs in length.

FIG. 28B illustrates the nucleic acid sequence for the GHFc-GLA1×LhyFc-BV2 vector (SEQ ID NO:15).

FIG. 29A shows the vector map for the GLA-hyFc-BV2 vector which is 4131 base pairs in length.

FIG. 29B illustrates the nucleic acid sequence for the GLA-hyFc-BV2 vector (SEQ ID NO:46).

FIG. 30A shows the vector map for the GLA-2×L-hyFc-BV2 vector which is 4161 base pairs in length.

FIG. 30B illustrates the nucleic acid sequence for the GLA-2×L-hyFc-BV2 vector (SEQ ID NO:47).

FIG. 31A shows the vector map the GLA-3×L-hyFc-BV2 vector which is 4176 base pairs in length.

FIG. 31B illustrates the nucleic acid sequence for the GLA-3×L-hyFc-BV2 vector (SEQ ID NO:48).

FIG. 32 shows the amino acid sequences of the encoded polypeptides: GLA-hyFc (SEQ ID NO:49), GLA-1×L-hyFc (SEQ ID NO:50), GLA-2×L-hyFc (SEQ ID NO:51) and GLA-3×L-hyFc (SEQ ID NO:52), respectively.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the phrase “and/or” as used in a phrase such as “A and/or B” herein is intended to include both A and B; A or B; A (alone); and B (alone). Likewise, the phrase “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 25%, 20%, 15%, 10%, 9%, 8%, 7%, 6%, 5%, 4%, 3%, 2%, 1%, 0.5%, or 0.05% of a given value or range.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments.

The term “composition” is intended to encompass a product containing the specified ingredients (e.g., a nucleic acid encoding a protein or other BANEM) in, optionally, the specified amounts.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants and other tonicity adjusting agents, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition is administered intravenously. Saline solutions and aqueous dextrose and glycerol solutions can also be employed as liquid carriers, particularly for injectable solutions, such as Ringer's solution or lactated Ringer's solution. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990).

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

The term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.), and polyols (e.g., mannitol, sorbitol, etc.). See, also, Remington and Gennaro, Remington's Pharmaceutical Sciences (18th ed. 1990), which is hereby incorporated by reference in its entirety.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blending. Such compounds include e.g., lactose, starch, mannitol, sorbitol, dextrose, cellulose, calcium phosphate, lactose; starch, mannitol, dextrates, amylose, cellulose, and the like.

As used herein, the term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject. In one embodiment, the subject is a mammal. In one embodiment, the subject is a human.

As used herein, the terms “treat,” “treating,” and “treatment” refer to the eradication or amelioration of a disease or disorder, or of one or more symptoms associated with the disease or disorder. In general, treatment occurs after the onset of the disease or disorder. In certain embodiments, the terms refer to minimizing the spread or worsening of the disease or disorder resulting from the administration of one or more prophylactic or therapeutic agents to a subject with such a disease or disorder.

As used herein, the term “administration” refers to the act of giving a composition as described herein to a subject. Exemplary routes of administration to the human body can be through the mouth (oral), skin (transdermal, topical), nose (nasal), lungs (inhalant), oral mucosa (buccal), by injection (e.g., intravenously, subcutaneously, intratumorally, intraocular, intraperitoneally, etc.), by transplantation (e.g. through an incision surgery) and the like.

As used herein, the terms “prevent,” “preventing,” and “prevention” refer to the prevention of the onset, recurrence or spread of a disease or disorder, or of one or more symptoms thereof. In general, prevention occurs prior to the onset of the disease or disorder.

As used herein, and unless otherwise specified, the terms “manage,” “managing,” and “management” refer to preventing or slowing the progression, spread or worsening of a disease or disorder, or of one or more symptoms thereof. Sometimes, the beneficial effects that a subject derives from a prophylactic or therapeutic agent do not result in a cure of the disease or disorder.

As used herein, and unless otherwise specified, the term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

The term “fat pad” as used herein refers to any cushions made of a pocket of fascia and filled with fat deposits (e.g. fatty acids in fat cells) that are present in humans or mammalians.

The term “adipocytes” as used herein refers to the functional cell type of fat, or adipose tissue, that is found throughout the body, particularly under the skin. Adipocytes store and synthesize fat or triglycerides for energy, thermal regulation and cushioning against mechanical shock. Without bound by the theory, it appears that mesenchymal stem cells can differentiate into two types of lipoblasts, one that give rise to white adipocytes and the other to brown adipocytes. Both types of adipocytes store fat. Adipose tissue may be brown or white adipose tissue, derived from, for example, subcutaneous, omental/visceral, mammary, gonadal, periorgan or other adipose tissue site. In some embodiments, adipose tissue is subcutaneous white adipose tissue. The adipose tissue may be from any organism having fat tissue. In some embodiments, the adipose tissue is mammalian. In some embodiments, the adipose tissue is in a human subject. A convenient source of adipose tissue is liposuction surgery. In some embodiments, adipocyte cells as described herein are desired for autologous transplantation into a subject. In some embodiments, the adipose tissue can be isolated from that subject.

The term “pre-adipocyte” as used herein refers to adipocyte precursor cells that, under the action of hormones such as insulin and glucocorticoid, divide and differentiate into adipocytes. Morphologically, pre-adipocytes are fibroblast-looking (i.e., thin and spindle-shaped) and devoid of triglyceride vesicles in their cytoplasm. As compared to adipocytes, pre-adipocytes contain low levels of insulin receptor and relatively high levels of IGF-1 receptors for receiving mitogenic and differentiating signals.

The term “subcutaneous” refer to “under the skin,” i.e., administered into the subcutis, the layer of skin directly below the dermis and epidermis (collectively referred to as the cutis), above muscle. In some embodiments, a composition as described herein is delivered to a subject subcutaneously, such as with the use of a standard needle and syringe. In some embodiments, the syringe is a pre-filled syringe. In some embodiments, a pen delivery device or autoinjector is used for subcutaneous delivery. Non-limiting examples of disposable pen delivery devices having applications in subcutaneous delivery include the SOLOSTAR™ pen (sanofi-aventis), the FLEXPEN™ (Novo Nordisk), and the KWIKPEN™ (Eli Lilly) and the SURECLICK™ Autoinjector (Amgen, Thousand Oaks, Calif.). In some embodiments, a composition as described herein is delivered to a subject subcutaneously, such as through transplantation in an incision surgery. In some embodiments, a subcutaneous incision is made in a subject to expose the fat tissue, and a solid medium (e.g., a sterile strip carrying effective amount of a therapeutic composition containing plasmid DNA) is placed into or near the fat tissue before the incision is closed up through a standard surgical procedure.

The term “incision” refers to a cut, opening, flap or penetration, typically in the course of minimally invasive or less-invasive surgery. An incision can be made with, for example, a knife, needle, blade, lancet, scalpel, laser, or other mechanism. In some embodiments, the incision is performed by lifting up the skin. In some embodiments, an incision extends beyond the dermal layer of a subject's skin. In some embodiments, the site of incision exposes adipocytes or pre-adipocytes. In some embodiments, the site of incision has damaged cells. In some embodiments, a composition described herein is administrated by incision.

The term “vector” refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Non-limiting examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The term “nucleic acid” refers to at least two or more ribo- or deoxy-ribonucleic acid base pairs (nucleotides/nucleosides) that are linked through a phosphoester bond or equivalent. Nucleic acids include polynucleotides. Nucleic acids include single, double or triplex, circular or linear, molecules. Exemplary nucleic acids include but are not limited to: RNA, DNA, cDNA, genomic nucleic acid, naturally occurring and non-naturally occurring nucleic acid, e.g., synthetic nucleic acid. Nucleic acids can be of various lengths. Nucleic acid lengths typically range from about 20 bases to 20 Kilobases (Kb), or any numerical value or range within or encompassing such lengths, 10 bases to 10 Kb, 1 to 5 Kb or less, 1000 to about 500 bases or less in length. Nucleic acids can also be shorter, for example, 100 to about 500 bases, or from about 12 to 24, 24 to 45, 45 to 90, 90 to 250, or about 250 to 500 bases in length. In some embodiments, a nucleic acid sequence has a length from about 10-20, 20-30, 30-50, 50-100, 100-150, 150-200, 200-250, 250-300, 300-400, 400-500, 500-1000, 1000-3000 bases. Shorter nucleic acids are commonly referred to as “oligonucleotides”. In some embodiment, nucleic acid sequences further include nucleotide and nucleoside substitutions, additions and deletions, as well as derivatized forms and fusion/chimeric sequences (e.g., encoding recombinant polypeptide. Nucleic acids can be produced using various techniques that includes, but are not limited to nucleic acid amplification, e.g., polymerase chain reaction (PCR). Nucleic acids can also be produced by chemical synthesis, such as solid phase phosphoramidite synthesis. The sequences produced can then be translated in vitro, or cloned into a plasmid and propagated and then expressed in a cell.

The term “CpG-reduced” as used herein refers to a nucleic acid sequence or expression vector that has less CpG di-nucleotides than present in the wild-type versions of the sequence or vector. “CpG-free” means the subject nucleic acid sequence or vector does not have any CpG di-nucleotides. An initial sequence, that contains CpG dinucleotides (e.g., wild-type version of an anti-SARS-CoV-2 antibody), may be modified to remove CpG dinucleotides by altering the nucleic acid sequence. Such CpG di-nucleotides can be suitably reduced or eliminated not just in a coding sequence, but also in the non-coding sequences, including, e.g., 5′ and 3′ untranslated regions (UTRs), promoter, enhancer, polyA, ITRs, introns, and any other sequences present in the nucleic acid molecule or vector. In certain embodiments, the nucleic acid sequences employed herein are CpG-reduced or CpG-free.

The term “plasmid” as used herein refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy. Plasmids may optionally comprise a selectable marker, such as an antibiotic resistance gene to help select for bacterial cells containing the plasmid and which ensures that the plasmid is retained in the bacterial cell. A plasmid as used herein may comprise a nucleic acid sequence encoding a heterologous gene, e.g., a gene encoding a branched chain amino acid catabolism enzyme. In some embodiment, the plasmid is used as a vector.

The term “mRNA” means “messenger-RNA” and relates to a “transcript” which is generated by using a DNA template and encodes a peptide or polypeptide. Typically, an mRNA comprises a 5′-UTR, a protein coding region, and a 3′-UTR. The term “mRNA” can include precursor mRNA and mature mRNA, either the full-length mRNA or its fragment. In some embodiment, mRNA may be generated by in vitro transcription from a DNA template. the stability and translation efficiency of RNA may be modified as required. In some embodiments, mRNA is stabilized and its translation increased by one or more modifications having a stabilizing effects and/or increasing translation efficiency of RNA. Such modifications are described, for example, in PCT/EP2006/009448 incorporated herein by reference. The modification maybe within the coding region, i.e. the sequence encoding the expressed peptide or protein, preferably without altering the sequence of the expressed peptide or protein, so as to increase the GC-content to increase mRNA stability and to perform a codon optimization and thereby enhance translation in cells.

The term “antibody,” “immunoglobulin,” or “Ig” is used interchangeably herein, and is used in the broadest sense and specifically encompasses, for example, individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a protein, a fragment of a protein, or an epitope of a protein. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments or antigen binding portions) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monoclonal antibody” or “mAb” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. In contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies. Monoclonal antibody as used herein is not limited to antibodies produced through hybridoma technology. A monoclonal antibody can be derived from a single clone, including any eukaryotic, prokaryotic, or phage clone, by any means available or known in the art.

The term “humanized monoclonal antibody” as used herein refers to that all or most of the amino acid sequences of the murine monoclonal antibodies (including the framework region sequence in the variable region), except complementarity-determining regions (CDR) are substituted by the amino acid sequences of human immunoglobulins, to reduce the immunogenicity of the murine monoclonal antibody by genetic engineering methods.

The term “glycosidase” as used herein refers to an agent that cleaves a covalent bond between sequential sugars in a glycan or between the sugar and the backbone moiety (e.g. between sugar and peptide backbone of glycoprotein). In some embodiments, a glycosidase is an enzyme. In certain embodiments, a glycosidase is a protein (e.g., a protein enzyme) comprising one or more polypeptide chains.

The term “galactosidase” as used herein refers to a class of enzymes that catalyze the hydrolysis and cleave terminal galactose residues. Galactosidases include “beta-galactosidase” that catalyzes the hydrolysis of beta-galactosides to form monosaccharides and “alpha-galactosidase” that catalyzes the hydrolysis of alpha-galactosides to form monosaccharides. Substrates of beta-galactosidases includes, but are not limited to, ganglioside GM1, lactosylceramides, lactose, and various glycoproteins.

The term “antigen” refers to a molecule that is capable of being bound specifically by an antibody, or otherwise provokes an immune response. This immune response may involve either antibody production, or the activation of specific immunologically-competent cells, or both. It is known in the art that any macromolecule, including virtually all proteins or peptides, can serve as an antigen.

The term “pathogen” refers to an organism or an infectious agent whose infection of cells of viable plant tissue elicits a disease response. In some embodiments, the pathogen is a bacteria. In some embodiments, the pathogen is a virus, particularly coronavirus.

The term “coronavirus” is a term of art which refers to an enveloped virus with a positive-sense single-stranded RNA genome and a helical symmetry. The genomic size of coronaviruses ranges from approximately 27 to 32 kilobases. Large Spike (S) glycoproteins protrude from the virus particle giving coronaviruses a distinctive corona-like appearance when visualized by electron microscopy. Coronaviruses infect a wide variety of species, including canine, feline, porcine, murine, bovine, avian and human. Coronaviruses typically bind to target cells through Spike-receptor interactions and enter cells by receptor mediated endocytosis or fusion with the plasma membrane. The Spike-receptor interaction is a strong determinant of species specificity.

The term “transfection” refers to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material.

The term “lipid” as used herein refers to a synthetic or naturally-occurring compound which is generally amphipathic and biocompatible. The lipids typically comprise a hydrophilic component and a hydrophobic component. Exemplary lipids include, for example, fatty acids, neutral fats, phosphatides, glycolipids, surface-active agents (surfactants), aliphatic alcohols, waxes, terpenes, and steroids. In some embodiments, the lipid is a phospholipid. Exemplary phospholipids that can form part of the liposomes as used herein include but are not limited to 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC).

The term “cationic lipid” includes lipids and synthetic lipids having both polar and non-polar domains and which are capable of being positively charged at or around physiological pH and which bind to polyanions, such as nucleic acids. In some embodiments, cationic lipids include saturated and unsaturated alkyl and alicyclic ethers and esters of amines, amides, or derivatives thereof. In some embodiments, cationic lipids comprise straight-chain, branched alkyl, alkenyl groups, or any combination of the foregoing. In some embodiments, cationic lipids contain from 1 to about 25 carbon atoms. In some embodiments, cationic lipids contain more than 25 carbon atoms. In some embodiments, straight chain or branched alkyl or alkene groups have six or more carbon atoms. In some embodiment, the cationic lipid is a cationic phospholipid. In some embodiments, the cationic lipid is N-[1-(2,3-dioleoyloxy)propyl]-N,N,N-trimethylammonium chloride (DOTAP).

The term “neutral lipid” refers to any of a number of lipid species which exist either in an uncharged or neutral zwitterionic form a physiological pH. Such lipids include, for example, diacylphosphatidylcholine, diacylphosphatidylethanolamine, ceramide, sphingomyelin, cephalin, cholesterol, cerebrosides and diacylglycerols. Certain neutral lipids, including cholesterol and other sterol derivatives, are known to increase the stability of liposomes and are referred to as “liposomes stabilizing lipids”.

The term “liposome” as used herein, refers to a vesicle composed of amphiphilic lipids arranged in at least one bilayer, e.g., one bilayer or a plurality of bilayers. Liposomes include unilamellar and multilamellar vesicles (MLV)that have a membrane formed from a lipophilic material and an aqueous interior. Multilamellar vesicles have more than one layer of membranes. Unilamellar vesicles have one single layer of membrane. The unilamellar vesicles may be large unilamellar vesicles (LUVs) or small unilamellar vesicles (SUVs). The term “large unilamellar vesicles” or “LUVs” as used herein means unilamellar vesicles having a diameter of between about 0.1 to 1 μm. The term “small unilamellar vesicles” or “SUV” as used herein means unilamellar vesicles having a diameter of less than 100 nm. The lipophilic material of liposome isolates the aqueous interior from an aqueous exterior. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes, thereby facilitating the delivery of therapeutic payloads on or inside the liposome. In some embodiment, the liposome used herein is small unilamellar vesicle (SUV). In some embodiment, the liposome used herein is multilamellar vesicle (MLV).

The term “cationic liposome” refers to liposomes that are made in whole or part from positively charged lipids, or more specifically a lipid that comprises both a cationic group and a lipophilic portion. The positively charged moieties of cationic lipids used in cationic liposomes provide advantageous structural features. For instance, the lipophilic portion of the cationic lipid is hydrophobic and thus may direct itself away from the aqueous interior of the liposome and associate with other nonpolar and hydrophobic species, or conversely, the cationic moiety may associate with polar molecules and species with which it can complex in the aqueous interior of the cationic liposome. The positively charged liposomes may interact with the negatively charged nucleic acid molecules to form a stable complex. Positively charged liposomes can bind to negatively charged cell surface constituents (e.g., heparin sulfate proteoglycans and integrins) and trigger cellular uptake mainly by endocytosis.

The term “neutral liposomes” refers to liposomes that contain lipid components that have an overall neutral charge at physiological pH. Neutral liposomes are less susceptible to interaction with negative constituents in the circulation after systemic delivery, compared to cationic liposomes. Cationic liposomes bind to and are taken up by endothelial cells after systemic delivery, while anionic and neutral liposomes are generally not.

As used herein, “empty liposomes” refers to liposomes that do not contain nucleic acid molecules but that may contain other bioactive molecules (e.g., liposomes that are only composed of the lipid molecules themselves, or only lipid molecules and a small molecule drug). In certain embodiments, empty liposomes are used with any of the methods or compositions described herein.

As used herein, “empty cationic micelles” refers to cationic micelles that do not contain nucleic acid molecules but that may contain other bioactive molecules (e.g., micelles that are only composed of lipid and surfactant molecules themselves, or only lipid and surfactant molecules and a small molecule drug). In certain embodiments, empty cationic micelles are used with any of the methods or compositions described herein.

As used herein, “empty cationic emulsions” refers to cationic emulsions or micro-emulsions that do not contain nucleic acid molecules but that may contain other bioactive molecules. In certain embodiments, empty cationic emulsions are used with any of the methods or compositions described herein.

The term “hyaluronidase” as used herein refers to an enzyme that decomposes hyaluronic acid. The hyaluronidase may be derived from mice, sheep, cattle, or humans. The hyaluronidase may be human hyaluronidase or recombinant human hyaluronidase. The term hyaluronidase as used herein includes its salts and derivatives which retain its enzymatic activity. A recombinant form of hyaluronidase for human injection, sold under the, trademark Hylenex® (Halozyme, Inc., San Diego, Calif.), is an FDA approved enzyme. Without bound by the theory, hyaluronidase hydrolyzes hyaluronic acid by cleaving the 0-1,4 bond between the glucosamine and glucuronic acid.

The term “isotonic” as used herein refers to a solution wherein the osmotic pressure gradient across the cell membrane is essentially balanced. An isotonic formulation is one which has essentially the same osmotic pressure as the blood of a subject. Isotonic formulations will generally have an osmotic pressure from about 250 mOsm/kg to 350 mOsm/kg.

The term “hypertonic” as used herein refers to a fluid having an osmotic concentration and a density greater than the osmotic concentration and density of blood of a subject. The physiologic, isotonic concentration of human blood is about 290 milli-osmols. In some embodiment, a hypertonic solution comprises a salt, such as a potassium salt, a sodium salt or a calcium salt. In some embodiment, a hypertonic solution comprises glucose and/or nonelectrolytes, such as mannitol. The term “hypertonic saline” as used herein refers to a solution of sodium chloride in water having a concentration of sodium chloride greater than 0.9% w/v.

The term “hypotonic” as used herein refers to a fluid having an osmotic concentration and a density lower than the osmotic concentration and density of blood of a subject. When a cell is immersed into a hypotonic solution, water flows into the cell in order to balance the concentration of the solutes. In some embodiments, the hypotonic solution comprises a salt. In one embodiment, the salt is sodium chloride. In one embodiment, the salt is potassium chloride. In one embodiment, the hypotonic solution comprises electrolytes. The term “hypotonic saline” as used herein refers to a solution of sodium chloride in water having an osmolality lower than 290 milli-osmols or a concentration of sodium chloride lower than 0.9% w/v.

As used herein, the term “alkyl” means a straight or branched saturated hydrocarbon chain containing from 1 to 30 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), or 5 to 23 carbon atoms (C₅-C₂₃ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkenyl” refers to a straight or branched hydrocarbon chain containing from 2 to 30 carbon atoms and containing at least one carbon-carbon double bond, for example 2 to 16 carbon atoms (C₂-C₁₆ alkyl), 2 to 14 carbon atoms (C₂-C₁₄ alkyl), 2 to 12 carbon atoms (C₂-C₁₂ alkyl), 2 to 10 carbon atoms (C₂-C₁₀ alkyl), 2 to 8 carbon atoms (C₂-C₈ alkyl), 2 to 6 carbon atoms (C₂-C₆ alkyl), 2 to 4 carbon atoms (C₂-C₄ alkyl), or 5 to 23 carbon atoms (C₅-C₂₃ alkyl). Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

### Methods and Compositions

Provided herein are compositions, systems, kits, and methods for treating a subject, and/or a subject's adipocytes, with a composition containing a nucleic acid sequence encoding a protein or a biologically active nucleic acid encoded molecule (BANEM), or a vector containing the nucleic acid sequence, wherein the treating comprises: a) injecting the composition into a subcutaneous (SC) region of the subject such that the at least one protein, or BANEM, is detectable in a blood, serum, or plasma sample from the subject; and/or b) injecting the composition into an SC region of the subject such that in-vivo transfected pre-adipocytes and/or adipocytes (e.g., cells of fat cell origin) are generated; c) performing the following: i) contacting adipocytes pre-adipocytes and/or adipocytes (e.g., cells of fat cell origin) from the subject ex-vivo with the composition such that ex-vivo transfected pre-adipocytes and/or adipocytes are generated, and ii) injecting the ex-vivo transfected pre-adipocytes and/or adipocytes into one or more SC regions of the subject; and/or d) implanting a solid medium carrying the composition into one or more subcutaneous regions of the subject.

In one embodiment, provided herein is a method of treating a subject. In one embodiment, provided herein is a method of treating the subcutaneous pre-adipocytes of a subject. In one embodiment, provided herein is a method of treating the subcutaneous adipocytes of a subject. In one embodiment, provided herein is a method of treating the subcutaneous pre-adipocytes and adipocytes of a subject.

In one embodiment, provided herein is a method comprising: treating a subject, and/or the subject's subcutaneous pre-adipocytes and/or adipocytes, with a composition, wherein the composition comprises, or consists essentially of, a nucleic acid sequence, or a vector containing the nucleic acid sequence, wherein the nucleic acid sequence encodes at least one protein (e.g., one, two, three, four, five, six, or more proteins), or at least one biologically active nucleic acid molecule (e.g., one, two, three, four, five, six, or more biologically active nucleic acid molecules), and wherein the treating comprises at least one of the following:

a) injecting the composition into one or more subcutaneous regions of the subject (e.g., fat pad, buttocks, stomach, etc.) such that the at least one protein, or the at least one biologically active nucleic acid encoded molecule, is detectable in a blood, serum, or plasma sample from the subject; and/or

b) injecting the composition into one or more subcutaneous regions of the subject such that a plurality of in-vivo transfected pre-adipocytes and/or adipocytes are generated in the subcutaneous region(s);

c) performing the following:
i) contacting a plurality of the pre-adipocytes and/or adipocytes from the subject ex-vivo with the composition such that a plurality of ex-vivo transfected pre-adipocytes and/or adipocytes are generated, and
ii) injecting at least some of the plurality of ex-vivo transfected pre-adipocytes and/or adipocytes into one or more subcutaneous regions of the subject; and/or

d) implanting a solid medium carrying the composition into one or more subcutaneous regions of the subject.

Nucleic Acid Containing Compositions

In one embodiment, the composition comprises a nucleic acid sequence. In one embodiment, the nucleic acid sequence is comprised in a vector. In some embodiments, the vector is a transfection vector. In some embodiments, the vector is an expression vector. In one embodiment, the vector comprises a non-viral vector. In one embodiment, the vector comprises a plasmid. In one embodiment, the plasmid is a Resistance plasmid. In one embodiment, the plasmid is a Fertility plasmid. In one embodiment, the plasmid is a Col plasmid. In one embodiment, the plasmid is a Degradative plasmid. In some embodiments, the vector comprising the nucleic acid sequence is not attached to or encapsulated in, any delivery agent.

In one embodiment, the nucleic acid sequence comprises DNA. In one embodiment, the nucleic acid sequence comprises plasmid DNA. In one embodiment, the nucleic acid sequence comprises linear DNA. In one embodiment, the nucleic acid sequence comprises circular DNA. In one embodiment, the nucleic acid sequence comprises supercoiled DNA.

In one embodiment, the nucleic acid comprises mRNA, and wherein the mRNA is optionally capped and composed of at least some modified bases that reduce immunogenicity.

In some embodiments, the vector comprises a nucleic acid sequence encoding for at least one protein. In one embodiment, the nucleic acid sequence encodes one protein. In one embodiment, the nucleic acid sequence encodes two proteins. In one embodiment, the nucleic acid sequence encodes three proteins. In one embodiment, the nucleic acid sequence encodes four proteins. In one embodiment, the nucleic acid sequence encodes five proteins. In one embodiment, the nucleic acid sequence encodes six proteins. In one embodiment, the nucleic acid sequence encodes seven proteins. In one embodiment, the nucleic acid sequence encodes eight proteins. In one embodiment, the nucleic acid sequence encodes nine proteins. In one embodiment, the nucleic acid sequence encodes ten proteins. In one embodiment, the nucleic acid sequence encodes twelve proteins. In one embodiment, the nucleic acid sequence encodes fourteen proteins. In one embodiment, the nucleic acid sequence encodes sixteen proteins. In one embodiment, the nucleic acid sequence encodes eighteen proteins. In one embodiment, the nucleic acid sequence encodes twenty or more proteins. In some embodiments, the two or more proteins encoded by the nucleic acid sequence are configured to function cooperatively. In particular embodiments, a first of the two or more proteins encoded by the nucleic acid sequence is an antibody heavy chain or an antigen binding fragment of the heavy chain, while a second of the two or more proteins encoded by the nucleic acid sequence is an antibody light chain or an antigen binding fragment of the light chain, where the two proteins form a functional antibody or antigen binding portion of an antibody.

In one embodiment, the nucleic acid sequence encodes one or more BANEMs. In one embodiment, the nucleic acid sequence encodes two BANEMs. In one embodiment, the nucleic acid sequence encodes three BANEMs. In one embodiment, the nucleic acid sequence encodes four BANEMs. In one embodiment, the nucleic acid sequence encodes five BANEMs. In one embodiment, the nucleic acid sequence encodes six BANEMs. In one embodiment, the nucleic acid sequence encodes seven BANEMs. In one embodiment, the nucleic acid sequence encodes eight BANEMs. In one embodiment, the nucleic acid sequence encodes nine BANEMs. In one embodiment, the nucleic acid sequence encodes ten BANEMs. In one embodiment, the nucleic acid sequence encodes twelve BANEMs. In one embodiment, the nucleic acid sequence encodes fourteen BANEMs. In one embodiment, the nucleic acid sequence encodes sixteen BANEMs. In one embodiment, the nucleic acid sequence encodes eighteen BANEMs. In one embodiment, the nucleic acid sequence encodes twenty or more BANEMs. In one embodiment, the BANEM comprises an RNA. In one embodiment, the BANEM comprises a therapeutic RNA. In one embodiment, the BANEM comprises an mRNA. In one embodiment, the BANEM comprises an siRNA. In one embodiment, the BANEM comprises an shRNA. In one embodiment, the BANEM comprises miRNA. In one embodiment, the BANEM comprises an shRNA. In one embodiment, the BANEM comprises piRNA. In one embodiment, the BANEM comprises an shRNA. In one embodiment, the BANEM comprises snoRNA. In one embodiment, the BANEM comprises an shRNA. In one embodiment, the BANEM comprises tsRNA. In one embodiment, the BANEM comprises an shRNA. In one embodiment, the BANEM comprises srRNA. In one embodiment, the BANEM comprises an shRNA. In one embodiment, the BANEM comprises a CRISPR single guide RNA sequence (sgRNA). In one embodiment, the BANEM comprises an antisense sequence.

In some embodiments, the vector encodes an mRNA. In one embodiment, the mRNA is a monocistronic mRNA that comprises only one ORF. In certain embodiments, the monocistronic mRNA encodes a peptide or protein comprising at least one epitope of a selected antigen (e.g., a pathogenic antigen or a tumor associated antigen). In other embodiments, the mRNA is a multicistronic mRNA that comprises two or more ORFs. In one embodiment, the multiecistronic mRNA encodes two or more peptides or proteins that can be the same or different from each other. In one embodiment, each peptide or protein encoded by a multicistronic mRNA comprises at least one epitope of a selected antigen.

In one embodiment, the vector comprises at least one expression cassette of at least one protein. In one embodiment, the vector comprises a single expression cassette. In one embodiment, the vector comprises single expression cassette of a single protein. In one embodiment, the vector comprises single expression cassette of a single monoclonal antibody (mAb). In one embodiment, the vector comprises two expression cassettes of a single protein. In one embodiment, the vector comprises two expression cassettes of a single mAb. In one embodiment, the vector comprises two expression cassettes of two therapeutic genes.

In some embodiments, the composition comprises one or more vectors. In one embodiment, each vector encodes one antibody. In one embodiment, the vector encodes two antibodies. In one embodiment, the vector encodes three antibodies. In one embodiment, the vector encodes four antibodies. In one embodiment, the vector encodes five antibodies. In one embodiment, the vector encodes six antibodies. In one embodiment, the vector encodes seven antibodies. In one embodiment, the vector encodes eight antibodies. In one embodiment, the vector encodes nine antibodies. In one embodiment, the vector encodes ten antibodies. In one embodiment, the vector encodes eleven antibodies. In one embodiment, the vector encodes twelve or more antibodies.

In some embodiments, the composition comprises one or more vectors. In some embodiments, the composition comprises two vectors, and each vector encodes one antibody. In some embodiments, the composition comprises one or more vectors. In some embodiments, the composition comprises two vectors, and each vector encodes two antibodies. In some embodiments, the composition comprises two vectors, and each vector encodes three antibodies. In some embodiments, the composition comprises two vectors, and each vector encodes four antibodies. As described herein, a vector encoding an antibody (or antigen binding fragment thereof) can encode both the heavy chain and light chain of the antibody (or variable regions of the heavy and light chains.)

In one embodiment, the vector comprises at least one expression cassette of at least one protein. In some embodiments, the at least one protein encoded by the nucleic acid molecule in the composition is responsible for lipid metabolism in the subject. In some embodiments, the at least one protein is responsible for lipid synthesis. In some embodiments, the at least one protein is responsible for lipid transportation. In some embodiments, the at least one protein is responsible for lipid storage. In some embodiments, the at least one protein is responsible for lipid degradation or consumption. In some embodiments, the at least one protein in an enzyme catalyzing one or more of the lipid biosynthesis, transportation and/or degradation pathways in the subject.

In some embodiments, the at least one protein encoded by the nucleic acid molecule in the composition is responsible for fat metabolism in the subject. In some embodiments, the at least one protein is responsible for fat synthesis. In some embodiments, the at least one protein is responsible for fat transportation. In some embodiments, the at least one protein is responsible for fat storage. In some embodiments, the at least one protein is responsible for fat degradation or consumption. In some embodiments, the at least one protein in an enzyme catalyzing the fat biosynthesis, transportation and/or degradation pathways in the subject.

In specific embodiment, the at least one protein comprises glycosidase. In specific embodiment, the at least one protein comprises galactosidase. In specific embodiment, the at least one protein comprises beta-galactosidase. In specific embodiment, the at least one protein comprises alpha-galactosidase. In specific embodiment, the at least one protein comprises a mixture of alpha-galactosidase and beta-galactosidase. In specific embodiment, the at least one protein comprises a glucocerebrosidase. In specific embodiment, the at least one protein comprises a beta-glucocerebrosidase. In specific embodiment, the at least one protein comprises sulfatase enzymes. In specific embodiment, the at least one protein comprises formylglycine-generating enzymes. In specific embodiment, the at least one protein comprises ceramidase. In specific embodiment, the at least one protein comprises acid ceramidase. In specific embodiment, the at least one protein comprises neutral ceramidase. In specific embodiment, the at least one protein comprises alkaline ceramidase 1, alkaline ceramidase 2, or alkaline ceramidase 3. In specific embodiment, the at least one protein comprises lipase. In specific embodiment, the at least one protein comprises pancreatic lipase. In specific embodiment, the at least one protein comprises hepatic lipase. In specific embodiment, the at least one protein comprises pharyngeal lipase. In specific embodiment, the at least one protein comprises alpha-N-acetylgalactosaminidase (other names: alpha-NAGA or alpha-galactosidase B). In specific embodiment, the at least one protein comprises alpha-L-fucosidase. In specific embodiment, the at least one protein comprises acid alpha-glucosidase (other names: alpha-1,4-glucosidase, acid maltase).

In some embodiments, the at least one protein is a galactosidase or glycosidase. In some embodiments, the at least one protein is selected from alpha-galactosidase A (GLA), GBA (beta-glucocerebrosidase), FGE (formylglycine-generating enzyme), ASAH (acid ceramidase), SMPD1 (sphingomyelin phosphodiesterase 1) or NPC1 (Niemann-Pick C1 cholesterol transporter) or NPC2 (Niemann-Pick C2 cholesterol transporter), and LAL. In some embodiments, the at least one protein is a lipase. In some embodiments, the at least one protein is a phospholipase.

In some embodiments, the at least one protein encoded by the nucleic acid molecule in the composition comprises an antibody or an antigen binding portion of an antibody that target a pathogenic antigen. In some embodiments, the antibody or antigen binding portion thereof targets an infectious pathogen. In some embodiments, the infectious pathogen is selected from bacteria, virus, fungus, and parasite. In some embodiments, the pathogen is a coronavirus (e.g., SARS, SARS-Cov-2, MERS) or influenza. In one embodiment, the pathogen is a SARS. In one embodiment, the pathogen is SARS-Cov-2. In one embodiment, the pathogen is a MERS. In one embodiment, the pathogen is influenza. In one embodiment, the pathogen is influenza A. In one embodiment, the pathogen is influenza B.

In some embodiment, the antibody or antigen binding portion thereof is an anti-viral antigen or antigen binding portion thereof. In some embodiments, the antibody or antigen binding portion thereof is a neutralizing antibody targeting a virus. In one embodiment, the antibody, or antigen binding portion thereof, is specific for SARS-CoV-2. In one embodiment, the antibody is an anti-CoV-2 monoclonal antibody. In one embodiment, the antibody, or antigen binding portion thereof, is specific for an influenza. In one embodiment, the antibody is an anti-influenza monoclonal antibody. In one embodiment, the antibody is the 5J8 monoclonal antibody. In one embodiment, the antibody, or antigen binding portion thereof, is specific for a cytokine. In one embodiment, the antibody is a humanized monoclonal antibody targeting human interleukin-5 (hIL-5). In one embodiment, the monoclonal antibody is mepolizumab.

In one embodiment, the at least one protein comprises at least one anti-SARS-CoV-2 monoclonal antibody, or antigen-binding portion thereof. In some embodiment, the anti-SARS-CoV-2 antibody, or antigen-binding portion thereof, comprises at least four, or at least eight, or at least 11, anti-SARS-CoV-2 antibodies and/or antigen-binding portions thereof. In one embodiment, the anti-SARS-CoV-2 comprises four antibodies. In one embodiment, the anti-SARS-CoV-2 comprises five antibodies. In one embodiment, the anti-SARS-CoV-2 comprises six antibodies. In one embodiment, the anti-SARS-CoV-2 comprises seven antibodies. In one embodiment, the anti-SARS-CoV-2 comprises eight antibodies. In one embodiment, the anti-SARS-CoV-2 comprises nine antibodies. In one embodiment, the anti-SARS-CoV-2 comprises ten antibodies. In one embodiment, the anti-SARS-CoV-2 comprises eleven antibodies. In one embodiment, the anti-SARS-CoV-2 comprises twelve antibodies. In specific embodiments, the at least one protein comprises one or more selected from the group consisting of: REGN10933, REGN10987, VIR-7831, LY-CoV1404, LY3853113, Zost 2355K, CV07-209K, C121L, Zost 2504L, CV38-183L, COVA215K, RBD215, CV07-250L, C144L, COVA118L, C135K, and B38.

In one embodiment, the at least one protein comprises REGN10933. In one embodiment, the at least one protein comprises REGN10987. In one embodiment, the at least one protein comprises VIR-7831. In one embodiment, the protein comprises LY-CoV1404. In one embodiment, the at least one protein comprises LY3853113. In at least one protein embodiment, the at least one protein comprises Zost 2355K. In one embodiment, the at least one protein comprises CV07-209K. In one embodiment, the at least one protein comprises C121L. In one embodiment, the at least one protein comprises Zost 2504L. In one embodiment, the at least one protein comprises CV38-183L. In one embodiment, the at least one protein comprises CV38-183L. In one embodiment, the at least one protein comprises COVA215K. In one embodiment, the at least one protein comprises RBD215. In one embodiment, the at least one protein comprises CV07-250L. In one embodiment, the at least one protein comprises C144L. In one embodiment, the protein comprises COVA118L. In one embodiment, the at least one protein comprises C135K. In one embodiment, the protein comprises B38. In one embodiment, the at least one protein comprises COVA118L. In some embodiments, the at least one protein is one or more selected from Table 5 and Table 7.

In alternative embodiments, the at least one protein encoded by the nucleic acid molecule in the composition comprises a pathogenic antigen. In particular embodiments, the pathogenic antigen is processed to remove or reduce pathogenicity (i.e., ability to cause a disease or symptom). In some embodiments, the pathogenic antigen retains antigenicity (i.e., ability to illicit immune response targeting the pathogen).

In some embodiment, the at least one protein encoded by the nucleic acid molecule in the composition is selected from the group consisting of: human growth hormone, G-CSF protein (e.g., human GCSF), erythropoietin, Etanercept, Bevacizumab, Rituximab, Adalimumab, Infliximab, Trastuzumab, Insulin, Insulin glargine, Epoetin alfa, Pegfilgrastim, Ranibizumab, Darbepoetin alfa, Interferon beta-1a, Interferon beta-1a (Rebif), Insulin aspart, Rhu insulin, Octocog alfa, Insulin lispro, Cetuximab, Peginterferon alfa-2a, Interferon beta-1b, Eptacog alfa, Insulin aspart, OnabotulinumtoxinA, Epoetin beta, Rec antihemophilic factor, Filgrastin, Insulin detemir, Natalizumab, Insulin (humulin), ACE2, anti-SARS-CoV-2, anti-flu, anti-HIV and/or anti-malaria, Palivizumab, and a-galactosidase A (GLA) (e.g., human GLA). In some embodiments, the at least one protein comprises human GLA.

In one embodiment, the at least one protein encoded by the nucleic acid molecule comprises GLA-hyFc (SEQ ID NO:49). In one embodiment, the protein comprises GLA-1×L-hyFc (SEQ ID NO:50). In one embodiment, the protein comprises GLA-2×L-hyFc (SEQ ID NO:51). In one embodiment, the protein comprises GLA-3×L-hyFc (SEQ ID NO:52). In one embodiment, the protein comprises a linker peptide comprising the amino acid sequence of SEQ ID NO:27. In one embodiment, the protein comprises a linker peptide comprising the amino acid sequence of SEQ ID NO:28. In one embodiment, the protein expression level is affected by the length of the linker. In one embodiment, longer linker improves the expression level of protein.

In some embodiments, the at least one protein encoded by the nucleic acid molecule in the composition comprises an antibody or an antigen binding portion of an antibody that targets an antigen associated with a diseased cell. In some embodiments, the at least one protein encoded by the nucleic acid molecule in the composition comprises an antibody or an antigen binding portion of an antibody listed in Column A of Table 4. In alternative embodiments, the at least one protein encoded by the nucleic acid molecule in the composition comprises an antigen associated with a diseased cell. In some embodiments, the at least one protein encoded by the nucleic acid molecule in the composition comprises a disease associated antigen listed in Column E of Table 4. In some embodiments, the antigen associated with the diseased cell is an antigen associated with a cancerous cell, such as a tumor cell. In some embodiments, the antigen that is targeted by the antibody or antigen binding portion thereof encoded by the nucleic acid in the composition is a tumor associated antigen. In some embodiments, the antigen that is encoded by the nucleic acid in the composition is a tumor associated antigen.

In specific embodiments, the at least one protein encoded comprises a linker peptide. In specific embodiment, the linker is 1× linker. In specific embodiment, the linker is a 2× linker. In specific embodiment, the linker comprises a sequence as set forth in SEQ ID NO: 27 or SEQ ID NO: 28.

In some embodiments, the vector further comprises one or more non-coding sequences. In one embodiment, the vector comprises an enhancer. In one embodiment, the enhancer is a murine CMV enhancer (M-CmvEnh) listed in Table 3B. In one embodiment, the enhancer is a human CMV enhancer (H-CmvEnh) listed in Table 3B. In some embodiment, the super-enhancer is selected from the enhancer sequences of hr3, SE1, SE2, SE3, SE4 or SE5 listed in Table 3B. In one embodiment, the plasmid comprises a super-enhancer.

In one embodiment, the vector comprises a promoter. In one embodiment, the promoter is selected from the promoter sequences of H-CMV, H-FerH, M-FABP2, H-CBOX1, H-REG1 and H-TDOX listed in Table 3B.

In one embodiment, the vector comprises at least one Matrix Attachment Region (MAR). In some embodiments, the MAR is selected from the MAR sequences of βGlo MAR and IFNβ S/MAR listed in Table 3B.

In one embodiment, the vector comprises a long terminal repeat (LTR). In some embodiments, the LTR is within an intron. In one embodiment, the LTR is selected from the LTR sequences of PPRV, MV, SNV and RU5 listed in Table 3B.

In one embodiment, the vector comprises a Nuclear Localization Sequence (NLS). In some embodiments, the vector comprises a MicroTubule-Associated Sequence (MTAS). In one embodiment, the plasmid comprises an NLS-MTAS as listed in Table 3B.

In specific embodiment, the vector comprises an enhancer. In specific embodiment, the enhancer comprises a sequence as set forth in SEQ ID NO: 16 or SEQ ID NO: 17. In specific embodiment, the vector as used herein comprises a promoter. In specific embodiment, the promoter comprises a sequence as set forth in SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26. In specific embodiment, the vector as used herein comprises a matrix-attachment region (MAR). In specific embodiment, the MAR comprises a sequence as set forth in SEQ ID NO: 29, SEQ ID NO: 30 or SEQ ID NO: 31. In specific embodiment, the vector as used herein comprises a super-enhancer. In specific embodiment, the super-enhancer comprises a sequence as set forth in SEQ ID NO: 32, SEQ ID NO: 33, SEQ ID NO: 34, SEQ ID NO: 35, SEQ ID NO: 36 or SEQ ID NO: 37. In specific embodiment, the vector as used herein comprises a long-term repeat (LTR) within intron. In specific embodiment, the LTR comprises a sequence as set forth in SEQ ID NO: 38, SEQ ID NO: 39, SEQ ID NO: 40 or SEQ ID NO: 41. In specific embodiment, the vector as used herein comprises a nucleus localization sequence-microtubule-associated sequence (NLS-MTAS). In specific embodiment, the NLS-MTAS further comprises a hydrophilic linker and modified neck domain peptide (NDP). In specific embodiment, the NLS-MTAS comprises a sequence as set forth in SEQ ID NO: 42, SEQ ID NO: 43, SEQ ID NO: 44 or SEQ ID NO: 45.

In one embodiment, the nucleic acid sequence or vector is CPG-free. In one embodiment, the nucleic acid sequence or vector is CPG-reduced. In some embodiments, the present disclosure employs CpG-reduced or CpG-free nucleic acid sequences and/or expression vectors. An initial sequence that contains CpG dinucleotides (e.g., wild-type version of an anti-SARS-CoV-2 antibody), may be modified to remove CpG dinucleotides by altering the nucleic acid sequence. Such CpG di-nucleotides can be suitably reduced or eliminated not just in a coding sequence, but also in the non-coding sequences, including, e.g., 5′ and 3′ untranslated regions (UTRs), promoter, enhancer, polyA, ITRs, introns, and any other sequences present in the nucleic acid molecule or vector. CpG di-nucleotides may be located within a codon triplet for a selected amino acid. There are five amino acids (serine, proline, threonine, alanine, and arginine) that have one or more codon triplets that contain a CpG di-nucleotide. All five of these amino acids have alternative codons not containing a CpG di-nucleotide that can be changed to, to avoid the CpG but still code for the same amino acid as shown in Table 1 below. Therefore, the CpG di-nucleotides allocated within a codon triplet for a selected amino acid may be changed to a codon triplet for the same amino acid lacking a CpG di-nucleotide.

In one embodiment, 0.05-60 mg/mL of the vectors are present in the composition. In one embodiment, 0.05-0.5 mg/mL of the vectors are present in the composition. In one embodiment, 0.5-1 mg/mL of the vectors are present in the composition. In one embodiment, 1-10 mg/mL of the vectors are present in the composition. In one embodiment, 10-20 mg/mL of the vectors are present in the composition. In one embodiment, 20-30 mg/mL of the vectors are present in the composition. In one embodiment, 30-40 mg/mL of the vectors are present in the composition. In one embodiment, 40-50 mg/mL of the vectors are present in the composition. In one embodiment, 50-60 mg/mL of the vectors are present in the composition.

In one embodiment, the composition comprising a nucleic acid sequence encoding a protein or other BANEM is free of any reagents that serve as aids to transfection. In one embodiment, the composition is free of any reagents that serve as aids to transfection (e.g., the composition comprises water and the nucleic acid sequence or vector containing the nucleic acid sequence, and no other ingredients or only inert other ingredients).

In one embodiment, the composition comprising a nucleic acid sequence encoding a protein or other BANEM is free of transfection agents. In one embodiment, the composition is free of viral transfection agents. In one embodiment, the composition is free of non-viral transfection agents. In one embodiment, the composition is free of chemical-based transfection agents, such as cationic polymers. In one embodiment, the composition is free of DNA transfection agents. In one embodiment, the composition is free of RNA transfection agents.

In one embodiment, the composition comprising a nucleic acid sequence encoding a protein or other BANEM further comprises one or more carriers, excipients, or diluents.

In some embodiments, the composition further comprises a sugar. In some embodiments, the sugar is selected from glucose, mannose, and dextrins. In some embodiments, the composition further comprises water for injection.

In some embodiments, the composition comprises a saline solution. In one embodiment, the composition comprises hypertonic saline. In one embodiment, the composition comprises the nucleic acid molecule dissolved in a Ringer's solution. In one embodiment, the composition comprises the nucleic acid molecule dissolved in a Lactated Ringer's solution. In one embodiment, the composition comprises the nucleic acid molecule dissolved in a hypotonic Ringer's solution. In one embodiment, the composition comprises the nucleic acid molecule dissolved in a hypotonic Lactated Ringer's solution.

In one embodiment, the composition comprises a hypertonic saline. In one embodiment, the composition comprises 3% (w/w) hypertonic saline. In one embodiment, the composition comprises 4% (w/w) hypertonic saline. In one embodiment, the composition comprises 5% (w/w) hypertonic saline. In one embodiment, the composition comprises 6% (w/w) hypertonic saline. In one embodiment, the composition comprises 7% (w/w) hypertonic saline. In one embodiment, the composition comprises 3% (w/w) hypertonic Ringer's solution. In one embodiment, the composition comprises 4% (w/w) hypertonic Ringer's solution. In one embodiment, the composition comprises 5% (w/w) hypertonic Ringer's solution. In one embodiment, the composition comprises 6% (w/w) hypertonic Ringer's solution. In one embodiment, the composition comprises 7% (w/w) hypertonic Ringer's solution. In one embodiment, the hypertonic solution is a 2× isotonic Ringer's solution, 2.5× isotonic Ringer's solution, 3× isotonic Ringer's solution, 4× isotonic Ringer's solution, 5× isotonic Ringer's solution, 6× isotonic Ringer's solution, or 7× isotonic Ringer's solution.

In one embodiment, the composition comprising a nucleic acid sequence encoding a protein or other BANEM further comprises a second active ingredient. In alternative embodiments, the composition comprising a nucleic acid sequence encoding a protein or other BANEM is used in combination with a second composition (e.g., a pre-treatment composition as described herein) comprising a second active ingredient. In some embodiments, the second active ingredient aids expression of the nucleic acid encoding the protein or other BANEM in the composition. In some embodiments, the second active ingredient reduces degradation of the nucleic acid encoding the protein or other BANEM in the composition. In some embodiments, the second active ingredient increases transportation of the nucleic acid encoding the protein or other BANEM into the cells. In some the second active ingredient increases permeability of cell membranes. In some embodiments, the second active ingredient is an anti-inflammation agent. In specific embodiments, the second ingredient is selected from lipids, dexamethasone, hyaluronidase, chloroquine, and TGF-β3.

In specific embodiments, the composition comprising a nucleic acid sequence encoding a protein or other BANEM further comprises hyaluronidase. In specific embodiments, hyaluronidase is comprised in the composition in the amount of at least 10 units, at least 20 units, at least 30 units, at least 40 units, at least 50 units, at least 60 units, at least 70 units, at least 80 units, at least 90 units, at least 100 units, at least 120 units, at least 140 units, at least 150 units, at least 200 units, or at least 300 units.

Methods of Administration

In one embodiment, provided herein is a method comprising: treating a subject, and/or the subject's subcutaneous pre-adipocytes and/or adipocytes, with a composition, wherein the composition comprises, or consists essentially of, a nucleic acid sequence, or a vector containing the nucleic acid sequence, wherein the nucleic acid sequence encodes at least one protein (e.g., one, two, three, four, five, six, or more proteins), or at least one biologically active nucleic acid molecule (e.g., one, two, three, four, five, six, or more biologically active nucleic acid molecules), and wherein the treating comprises at least one of the following:

a) injecting the composition into one or more subcutaneous regions of the subject (e.g., fat pad, buttocks, stomach, etc.) such that the at least one protein, or the at least one biologically active nucleic acid encoded molecule, is detectable in a blood, serum, or plasma sample from the subject; and/or

b) injecting the composition into one or more subcutaneous regions of the subject such that a plurality of in-vivo transfected pre-adipocytes and/or adipocytes are generated in the subcutaneous region(s);

c) performing the following:
i) contacting a plurality of the pre-adipocytes and/or adipocytes from the subject ex-vivo with the composition such that a plurality of ex-vivo transfected pre-adipocytes and/or adipocytes are generated, and
ii) injecting at least some of the plurality of ex-vivo transfected pre-adipocytes and/or adipocytes into one or more subcutaneous regions of the subject; and/or

d) implanting a solid medium carrying the composition into one or more subcutaneous regions of the subject.

In one embodiment, provided herein is a method comprising: treating a subject, and/or the subject's subcutaneous pre-adipocytes and/or adipocytes, with a composition, wherein the composition comprises, or consists essentially of, a nucleic acid sequence, or a vector containing the nucleic acid sequence, wherein the nucleic acid sequence encodes at least one protein (e.g., one, two, three, four, five, six, or more proteins), or at least one biologically active nucleic acid molecule (e.g., one, two, three, four, five, six, or more biologically active nucleic acid molecules), and wherein the treating comprises injecting the composition into one or more subcutaneous regions of the subject (e.g., fat pad, buttocks, stomach, etc.) such that the at least one protein, or the at least one biologically active nucleic acid encoded molecule, is detectable in a blood, serum, or plasma sample from the subject.

In one embodiment, provided herein is a method comprising: treating a subject, and/or the subject's subcutaneous pre-adipocytes and/or adipocytes, with a composition, wherein the composition comprises, or consists essentially of, a nucleic acid sequence, or a vector containing the nucleic acid sequence, wherein the nucleic acid sequence encodes at least one protein (e.g., one, two, three, four, five, six, or more proteins), or at least one biologically active nucleic acid molecule (e.g., one, two, three, four, five, six, or more biologically active nucleic acid molecules), and wherein the treating comprises injecting the composition into one or more subcutaneous regions of the subject such that a plurality of in-vivo transfected pre-adipocytes and/or adipocytes are generated in the subcutaneous region(s).

In one embodiment, provided herein is a method comprising: treating a subject, and/or the subject's subcutaneous pre-adipocytes and/or adipocytes, with a composition, wherein the composition comprises, or consists essentially of, a nucleic acid sequence, or a vector containing the nucleic acid sequence, wherein the nucleic acid sequence encodes at least one protein (e.g., one, two, three, four, five, six, or more proteins), or at least one biologically active nucleic acid molecule (e.g., one, two, three, four, five, six, or more biologically active nucleic acid molecules), and wherein the treating comprises:

i) contacting a plurality of the pre-adipocytes and/or adipocytes from the subject ex-vivo with the composition such that a plurality of ex-vivo transfected pre-adipocytes and/or adipocytes are generated, and

ii) injecting at least some of the plurality of ex-vivo transfected pre-adipocytes and/or adipocytes into one or more subcutaneous regions of the subject.

In one embodiment, the method comprises the injection of ex-vivo transfected pre-adipocytes or adipocytes into a subject. In one embodiment, the method further comprises the use of ex-vivo transfected pre-adipocytes or adipocytes as cell therapies. In some embodiments, the pre-adipocytes or adipocytes are isolated from a subject, ex-vivo transfected and then transferred back to the same subject. In alternative embodiments, the pre-adipocytes or adipocytes are isolated from a donor subject, ex-vivo transfected, and then transferred to a different recipient subject.

In one embodiment, the ex-vivo transfected pre-adipocytes and/or adipocytes are enlarged such that, on average, they contain at least 60 pg of lipid (e.g., at least 60 . . . 70 . . . 90 . . . 100 . . . 110 ug . . . etc.). In further embodiments, the plurality of the ex-vivo transfected pre-adipocytes and/or adipocytes are enlarged such that, on average, they contain at least 80 ug of lipid (e.g., at least 90 . . . 100 . . . 110 ug . . . etc.). In further embodiments, the plurality of the ex-vivo transfected pre-adipocytes and/or adipocytes are enlarged such that, on average, they have a diameter of at least 70 μm (e.g., at least 70 . . . 80 . . . 90 . . . 100 . . . 110 μm). In additional embodiments, the plurality of the ex-vivo transfected pre-adipocytes and/or adipocytes are enlarged such that, on average, they have a diameter of at least 90 μm (e.g., at least 90 . . . 100 . . . 110 . . . 120 μm).

In certain embodiments, the transfected and enlarged adipocytes contain, on average, at least 60 μg of lipid (e.g., 60 . . . 70 . . . 80 . . . 90 . . . ug). In some embodiments, the plurality transfected pre-adipocytes and/or adipocytes contain, on average, at least 80 μg of lipid. In other embodiments, the transfected pre-adipocytes and/or adipocytes have, on average, a diameter of at least 70 μm. In additional embodiments, the transfected pre-adipocytes and/or adipocytes have, on average, a diameter of at least 90 μm. In additional embodiments, the transfected pre-adipocytes and/or adipocytes are derived from one or more subcutaneous regions of a subject.

In one embodiment, the plurality of ex-vivo transfected pre-adipocytes and/or adipocytes are enlarged such that, on average, they contain at least 60 μg of lipid. In one embodiment, they contain at least 65 μg of lipid. In one embodiment, they contain at least 70 μg of lipid. In one embodiment, they contain at least 70 μg of lipid. In one embodiment, they contain at least 75 μg of lipid. In one embodiment, they contain at least 80 μg of lipid. In one embodiment, they contain at least 85 μg of lipid. In one embodiment, they contain at least 90 μg of lipid. In one embodiment, they contain at least 95 μg of lipid. In one embodiment, they contain at least 100 μg of lipid. In one embodiment, they contain at least 110 μg of lipid. In one embodiment, they contain at least 120 μg of lipid. In one embodiment, they contain at least 130 μg of lipid. In one embodiment, they contain at least 150 μg of lipid. In one embodiment, they contain at least 200 μg of lipid.

In one embodiment, the plurality of ex-vivo transfected pre-adipocytes and/or adipocytes are enlarged such that, on average, they have a diameter of at least 70 μm. In one embodiment, they have a diameter of at least 75 μm. In one embodiment, they have a diameter of at least 80 μm. In one embodiment, they have a diameter of at least 85 μm. In one embodiment, they have a diameter of at least 90 μm. In one embodiment, they have a diameter of at least 95 μm. In one embodiment, they have a diameter of at least 100 μm. In one embodiment, they have a diameter of at least 120 μm. In one embodiment, they have a diameter of at least 150 μm. In one embodiment, they have a diameter of at least 200 μm. In one embodiment, they have a diameter of at least 250 μm.

In one embodiment, the plurality of ex-vivo transfected pre-adipocytes and/or adipocytes are enlarged such that, on average, they have a diameter of at least 90 μm.

In one embodiment, the ex-vivo transfected pre-adipocytes and/or adipocytes have normal sizes.

In one embodiment, provided herein is a method comprising: treating a subject, and/or the subject's subcutaneous pre-adipocytes and/or adipocytes, with a composition, wherein the composition comprises, or consists essentially of, a nucleic acid sequence, or a vector containing the nucleic acid sequence, wherein the nucleic acid sequence encodes at least one protein (e.g., one, two, three, four, five, six, or more proteins), or at least one biologically active nucleic acid molecule (e.g., one, two, three, four, five, six, or more biologically active nucleic acid molecules), and wherein the treating comprises implanting a solid medium carrying the composition into one or more subcutaneous regions of the subject.

In some embodiments, the treating comprises implanting a solid medium carrying the composition into one or more subcutaneous regions of the subject, and wherein the at least one protein, or the at least one biologically active nucleic acid encoded molecule, is detectable in a blood, serum, or plasma sample from the subject. In some embodiments, the treating comprises implanting a solid medium carrying the composition into one or more subcutaneous regions of the subject, and wherein a plurality of in-vivo transfected pre-adipocytes and/or adipocytes are generated in the subcutaneous region(s).

In some embodiments, the implanting is performed via an incision surgery. In some embodiments, the solid medium carrying the composition is a biocompatible membrane. In some embodiments, the solid medium carrying the composition is a biocompatible hydrogel. In some embodiments, the solid medium carrying the composition comprises nanoparticles. In some embodiments, the solid medium carrying the composition is configured for slow release of the composition at the site of implantation. In some embodiments, the site of implantation comprises at least one fat tissue. In some embodiments, the site of implantation comprises at least one fat cell. In some embodiments, the site of implantation comprises at least one pre-adipocyte. In some embodiments, the site of implantation comprises at least one adipocyte.

According to the present disclosure, the injecting in a), b), or c) ii) or the implanting in d) can be performed once or multiple times to a single subject. In one embodiment, the injecting in a), b), or c) ii) or the implanting in d) is performed once. In one embodiment, the injecting in a), b), or c) ii) or the implanting in d) is performed twice. In one embodiment, the injecting in a), b), or c) ii) or the implanting in d) is performed three times. In one embodiment, the injecting in a), b), or c) ii) or the implanting in d) is performed four times. In one embodiment, the injecting in a), b), or c) ii) or the implanting in d) is performed five times. In one embodiment, the injecting in a), b), or c) ii) or the implanting in d) is performed six times. In one embodiment, the injecting in a), b), or c) ii) or the implanting in d) is performed seven times. In one embodiment, the injecting in a), b), or c) ii) or the implanting in d) is performed eight times. In one embodiment, the injecting in a), b), or c) ii) or the implanting in d) is performed nine times. In one embodiment, the injecting in a), b), or c) ii) or the implanting in d) is performed ten times. In one embodiment, the injecting in a), b), or c) ii) or the implanting in d) is performed more than ten times.

In some embodiments, the injecting in a), b), or c) ii) or the implanting in d) is performed multiple times at a frequency of once per day, or once per every two days, or once per every three days, or once per every four days, or once per every five days, or once per every six days, or once per week, or biweekly, or monthly, or once every two months, or once every three months, or once every four months, or once every five months, or twice a year, or annually, or other repeating frequencies.

In one embodiment, 1-1000 mg of the vectors are administrated. In one embodiment, 1-50 mg of the vectors are administrated. In one embodiment, 50-100 mg of the vectors are administrated. In one embodiment, 100-200 mg of the vectors are administrated. In one embodiment, 200-300 mg of the vectors are administrated. In one embodiment, 300-400 mg of the vectors are administrated. In one embodiment, 400-500 mg of the vectors are administrated. In one embodiment, 500-600 mg of the vectors are administrated. In one embodiment, 600-700 mg of the vectors are administrated. In one embodiment, 700-800 mg of the vectors are administrated. In one embodiment, 800-900 mg of the vectors are administrated. In one embodiment, 900-1000 mg of the vectors are administrated.

In one embodiment, a composition comprising the nucleic acid encoding at least one protein or other BANEM or a composition comprising ex-vivo transfected adipocytes or preadipocytes is injected or transplanted subcutaneously to a subject. In some embodiments, the composition is injected or transplanted subcutaneously and become accessible to at least one fat tissue in the subject. In some embodiments, the composition is injected or transplanted subcutaneously and become accessible to a population of preadipocytes in the subject. In some embodiments, the composition is injected or transplanted subcutaneously and become accessible to a population of adipocytes in the subject.

In one embodiment, the composition comprising the nucleic acid encoding at least one protein or other BANEM or a composition comprising ex-vivo transfected adipocytes or preadipocytes is injected or transplanted into one or more subcutaneous regions of a subject. In one embodiment, the composition is injected or transplanted into two subcutaneous regions. In one embodiment, the composition is injected or transplanted into three subcutaneous regions. In one embodiment, the composition is injected or transplanted into four subcutaneous regions. In one embodiment, the composition is injected or transplanted into five subcutaneous regions. In one embodiment, the composition is injected or transplanted into six subcutaneous regions. In one embodiment, the composition is injected or transplanted into seven subcutaneous regions. In one embodiment, the composition is injected or transplanted into eight or more subcutaneous regions.

In one embodiment, the subcutaneous region is in a fat pad. In one embodiment, the subcutaneous region is in buttock. In one embodiment, the subcutaneous region is in stomach.

In one embodiment, the composition is implanted at site of incision. In one embodiment, the incision is at the dermal layer of a subject's skin. In one embodiment, the incision extends beyond the dermal layer of a subject's skin. In one embodiment, the site of incision exposes adipocytes or pre-adipocytes. In one embodiment, the composition contacts the adipocytes or pre-adipocytes at site of incision. In one embodiment, the composition contacts basal cells at site of incision. In one embodiment, the composition contacts melanocytes at site of incision.

In one embodiment, the injecting in a), b), or c) ii) is into a fat pad of the subject. In one embodiment, the injecting is into a first fat pad of the subject. In one embodiment, the injecting is into a second fat pad of the subject. In one embodiment, the injecting is into a third fat pad of the subject. In one embodiment, the injecting is into a fourth fat pad of the subject. In one embodiment, the injecting is into a fifth fat pad of the subject. In one embodiment, the injecting in a), b), or c) ii) is into the same fat pad of the subject. In one embodiment, the injecting in a), b), or c) ii) is into different fat pads of the subject.

In one embodiment, the injecting in a), b), or c) ii) or the implantation in d) is performed at a plurality of sites in the subject. In additional embodiments, the injecting in a) and/or b) causes the subject to receive between 1 and 60 micrograms (e.g., 1.0 . . . 10 . . . 20 . . . 30 . . . 40 . . . 50 . . . or 60 micrograms), or between 0.00001 and 1.0 micrograms (e.g., 0.00001 . . . 0.0001 . . . 0.001 . . . 0.01 . . . 0.1 . . . and 1.0 micrograms), per microliter of the composition (e.g., aqueous composition) of the nucleic acid sequence, or the vector containing the nucleic acid sequence.

In one embodiment, wherein injection in a) and/or b) causes the subject to receive between 1 and 60 micrograms (μg), or between 0.00001 and 1.0 micrograms (μg), per microliter (μL) of the composition of the nucleic acid sequence, or the vector containing the nucleic acid sequence. In one embodiment, the subject receives 1 to 5 μg/μL of nucleic acid in the composition. In one embodiment, the subject receives 0.00001 to 0.0001 μg/μL of nucleic acid in the composition. In one embodiment, the subject receives 0.0001 to 0.001 μg/μL of nucleic acid in the composition. In one embodiment, the subject receives 0.001 to 0.01 μg/μL of nucleic acid in the composition. In one embodiment, the subject receives 0.01 to 0.1 μg/μL of nucleic acid in the composition. In one embodiment, the subject receives 0.1 to 1 μg/μL of nucleic acid in the composition. In one embodiment, the subject receives 1 to 5 μg/μL of nucleic acid in the composition. In one embodiment, the subject receives 5 to 10 μg/μL of nucleic acid in the composition. In one embodiment, the subject receives 10 to 15 μg/μL of nucleic acid in the composition. In one embodiment, the subject receives 15 to 20 μg/μL of nucleic acid in the composition. In one embodiment, the subject receives 20 to 25 μg/μL of nucleic acid in the composition. In one embodiment, the subject receives 25 to 30 μg/μL of nucleic acid in the composition. In one embodiment, the subject receives 30 to 35 μg/μL of nucleic acid in the composition. In one embodiment, the subject receives 35 to 40 μg/μL of nucleic acid in the composition. In one embodiment, the subject receives 40 to 45 μg/μL of nucleic acid in the composition. In one embodiment, the subject receives 45 to 50 μg/μL of nucleic acid in the composition. In one embodiment, the subject receives 50 to 60 μg/μL of nucleic acid in the composition. In one embodiment, the subject receives 1 μg/μL of nucleic acid in the composition.

In one embodiment, the subject receives 2 μg/μL of nucleic acid in the composition. In one embodiment, the subject receives 4 μg/μL of nucleic acid in the composition. In one embodiment, the subject receives 8 μg/μL of nucleic acid in the composition.

In one embodiment, the injection in a), b), or c) ii) or the implantation of d) is performed at a plurality of sites in the subject. In one embodiment, the injection is performed at a single site in the subject. In one embodiment, the injection is performed at two sites in the subject. In one embodiment, the injection is performed at two sites in the subject.

Pre-Treatment

In one embodiment, the methods further comprise one or more pre-treatment process, prior to the injecting in a), b), or c) ii) or the implantation of d).

In one embodiment, the methods further comprise, prior to the injecting in a), b), or c) ii) or the implantation of d) administering a pre-treatment solution.

In one embodiment, the pre-treatment solution comprises cationic liposomes. In one embodiment, the pre-treatment solution comprises neutral liposomes. In one embodiment, the pre-treatment solution comprises both cationic liposomes and neutral liposomes. In one embodiment, the liposome comprises Small Unilamellar Vesicles (SUV). In one embodiment, the liposome comprises Multilamellar Vesicles (MLV). In one embodiment, the pre-treatment solution comprises SUV. In one embodiment, the pre-treatment solution comprises MLV. In one embodiment, the pre-treatment solution comprises SUV. In one embodiment, the pre-treatment solution comprises a mixture of SUV and MLV. In one embodiment, the pre-treatment solution comprises a mixture of cationic SUV and neutral MLV. In one embodiment, the pre-treatment solution comprises a mixture of DOTAP SUV and DMPC MLV.

In one embodiment, the pre-treatment solution comprises a micelle. In one embodiment, the pre-treatment solution comprises an emulsion. In one embodiment, the pre-treatment solution comprises an empty micelle. In one embodiment, the pre-treatment solution comprises an empty emulsion.

In one embodiment, the pre-treatment solution comprises an anti-inflammatory agent (AIL). In some embodiments, the anti-inflammatory agent is selected from the group consisting of: dexamethasone, dexamethasone palmitate, a dexamethasone fatty acid ester, Docosahexaenoic Acid (DHA), Eicosapenaenoic Acid (EPA), Alpha Linolenic Acid (ALA), Lipoxin A4 (LA4), 15-deoxy-12,14-Prostaglandin J2 (15d), Arachidonic Acid (AA), Docosapentaenoic Acid (DPA), Retinoic Acid (RA), Diallyl Disulfide (DADS), Oleic Acid (OA), Alpha Tocopherol (AT), Sphingosine-1-Phosphate (S-1-P), Palmitoyl Sphingomyelin (SPH), an anti-TNFa antibody or antigen binding fragment thereof, a heparinoid, N-Acetyl-De-O-Sulfated Heparin, Chloroquine, and TGF-β3. In some embodiments, the anti-inflammatory agent is a lymphocyte depletion agent.

In one embodiment, the pre-treatment solution comprises liposome and anti-inflammatory agent. In one embodiment, the liposome and anti-inflammatory agent are injected together. In one embodiment, the liposome and anti-inflammatory agent are injected separated. In one embodiment, the anti-inflammatory agent is injected before the liposome. In one embodiment, the anti-inflammatory agent is injected after the liposome.

In one embodiment, the pre-treatment solution comprises Chloroquine.

In one embodiment, the pre-treatment solution comprises TGF-β3. In some embodiments, the pre-treatment solution comprises at least 10 ng, at least 20 ng, at least 30 ng, at least 40 ng, at least 50 ng, at least 100 ng, at least 150 ng, at least 200 ng, at least 250 ng, at least 300 ng, at least 500 ng TGF-β3.

In one embodiment, the pre-treatment solution comprises one or more lymphocyte depleting agent. In some embodiments, the lymphocyte depleting agent comprises one or more antibodies. In some embodiments, the lymphocyte depleting agent reduces matured B cell and/or T cell from the subject after administration.

In one embodiment, the pre-treatment solution comprises dexamethasone and/or dexamethasone palmitate. In one embodiment, the dexamethasone is water-soluble dexamethasone. In one embodiment, the dexamethasone is complexed to cyclodextrin to make it soluble in water. In one embodiment, the dexamethasone palmitate is dexamethasone 21-palmitate. In one embodiment, the dexamethasone palmitate is attached to a liposome. In one embodiment, the dexamethasone 21-palmitate is attached to a liposome. In one embodiment, the dexamethasone 21-palmitate is attached to a cationic liposome. In one embodiment, the dexamethasone 21-palmitate is attached to a neutral liposome. In one embodiment, the pre-treatment solution comprises liposome comprising dexamethasone palmitate. In one embodiment, the pre-treatment solution comprises cationic liposome comprising dexamethasone palmitate.

In one embodiment, the pre-treatment solution comprises neutral liposome comprising dexamethasone palmitate. In one embodiment, the pre-treatment solution comprises a mixture of cationic liposome comprising dexamethasone palmitate and neutral liposome comprising dexamethasone palmitate. In one embodiment, the pre-treatment solution comprises a mixture of cationic SUV comprising dexamethasone palmitate and neutral MLV comprising dexamethasone palmitate. In one embodiment, the pre-treatment solution comprises a mixture of DOTAP SUV comprising dexamethasone palmitate and DMPC MLV comprising dexamethasone palmitate.

In one embodiment, the pre-treatment solution is free or essentially free of nucleic acid.

In one embodiment, the liposome and dexamethasone are injected together. In one embodiment, the liposome and dexamethasone palmitate are injected together. In one embodiment, the liposome and dexamethasone are injected separately. In one embodiment, the dexamethasone is injected before the liposome. In one embodiment, the dexamethasone is injected after the liposome.

In one embodiment, the administration of the cationic and/or neutral liposomes is performed intravenously or subcutaneously. In one embodiment, the liposomes are administrated intravenously. In one embodiment, the liposomes are administrated subcutaneously. In one embodiment, the liposomes are administrated intranasally. In one embodiment, the liposomes are administrated intraperitoncally. In one embodiment, the liposomes are administrated intramuscularly. In one embodiment, the liposomes are administrated intratracheally. In one embodiment, the liposomes are administrated intradermally,

In one embodiment, the pre-treatment process further comprises injecting a nucleic acid. In one embodiment, the pre-treatment process further comprises injecting a DNA. In one embodiment, the pre-treatment process further comprises injecting a plasmid DNA. In one embodiment, the pre-treatment process further comprises injecting a nucleic acid intravenously. In one embodiment, the pre-treatment process further comprises injecting a plasmid DNA intravenously.

In one embodiment, the pre-treatment process comprises injecting a liposome, followed by injecting a nucleic acid. In one embodiment, the pre-treatment process comprises injecting a liposome intravenously, followed by injecting a nucleic acid intravenously. In one embodiment, the pre-treatment process comprises injecting a liposome comprising dexamethasone intravenously, followed by injecting a nucleic acid intravenously. In one embodiment, the pre-treatment process comprises injecting a mixture of cationic liposome comprising dexamethasone and a neutral liposome comprising dexamethasone intravenously, followed by injecting a nucleic acid intravenously.

In some embodiments, an agent, such as an anti-inflammatory agent or bioactive lipid, is used to increase the expression level and/or duration of any the therapeutic protein (or biologically active nucleic acid molecules) expressed from the vectors in the methods herein. In certain embodiments, anti-inflammatory agents (AILs) and bioactive lipids in Table 6 below can be used in the compositions and pre-treatment solutions herein. In some embodiments, the anti-inflammatory agent is selected from the group consisting of: dexamethasone, dexamethasone palmitate, a dexamethasone fatty acid ester, Docosahexaenoic Acid (DHA), Eicosapenaenoic Acid (EPA), Alpha Linolenic Acid (ALA), Lipoxin A4 (LA4), 15-deoxy-12,14-Prostaglandin J2 (15d), Arachidonic Acid (AA), Docosapentaenoic Acid (DPA), Retinoic Acid (RA), Diallyl Disulfide (DADS), Oleic Acid (OA), Alpha Tocopherol (AT), Sphingosine-1-Phosphate (S-1-P), Palmitoyl Sphingomyelin (SPH), an anti-TNFa antibody or antigen binding fragment thereof, a heparinoid, and N-Acetyl-De-O-Sulfated Heparin. In one embodiment, the anti-inflammatory agent is dexamethasone. In one embodiment, the anti-inflammatory agent is dexamethasone palmitate. In one embodiment, the anti-inflammatory agent is a dexamethasone fatty acid ester. In one embodiment, the anti-inflammatory agent is TGF-β3.

In certain embodiments, the present disclosure employs polycationic structures (e.g., empty cationic liposomes, empty cationic micelles, or empty cationic emulsions) not containing vector DNA, which are administered to a subject prior to administration of the composition containing the nucleic acid or vector containing the nucleic acid. In certain embodiments, the polycationic structures are cationic lipids and/or are provided as an emulsion. The present disclosure is not limited to the cationic lipids employed, which can be composed, in some embodiments, of one or more of the following: DDAB, dimethyldioctadecyl ammonium bromide; DPTAP (1,2-dipalmitoyl 3-trimethylammonium propane); DHA; prostaglandin, N-[1-(2,3-Dioloyloxy)propyl]-N,N,N-trimethylammonium methylsulfate; 1,2-diacyl-3-trimethylammonium-propanes, (including but not limited to, dioleoyl (DOTAP), dimyristoyl, dipalmitoyl, disearoyl); 1,2-diacyl-3-dimethylammonium-propanes, (including but not limited to, dioleoyl, dimyristoyl, dipalmitoyl, disearoyl) DOTMA, N-[1-[2,3-bis(oleoyloxy)]propyl]-N,N,N-trimethylammoniu-m chloride; DOGS, dioctadecylamidoglycylspermine; DC-cholesterol, 3.beta.-[N—(N′,N′-dimethylaminoethane)carbamoyl]cholesterol; DOSPA, 2,3-dioleoyloxy-N-(2(sperminecarboxamido)-ethyl)-N,N-dimethyl-1-propanami-nium trifluoroacetate; 1,2-diacyl-sn-glycero-3-ethylphosphocholines (including but not limited to dioleoyl (DOEPC), dilauroyl, dimyristoyl, dipalmitoyl, distearoyl, palmitoyl-oleoyl); beta-alanyl cholesterol; CTAB, cetyl trimethyl ammonium bromide; diC14-amidine, N-t-butyl-N′-tetradecyl-3-tetradecylaminopropionamidine; 14Dea2, O,O′-ditetradecanolyl-N-(trimethylammonioacetyl) diethanolamine chloride; DOSPER, 1,3-dioleoyloxy-2-(6-carboxy-spermyl)-propylamide; N,N,N′,N′-tetramethyl-N,N′-bis(2-hydroxylethyl)-2,3-dioleoyloxy-1,4-butan-ediammonium iodide; 1-[2-acyloxy)ethyl]2-alkyl (alkenyl)-3-(2-hydroxyethyl-) imidazolinium chloride derivatives such as 1-[2-(9(Z)-octadecenoyloxy)eth-yl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl)imidazolinium chloride (DPTIM); 1-[2-tetradecanoyloxy)ethyl]-2-tridecyl-3-(2-hydroxyeth-yl)imidazolium chloride (DMTIM) (e.g., as described in Solodin et al. (1995) Biochem. 43:13537-13544, herein incorporated by reference); 2,3-dialkyloxypropyl quaternary ammonium compound derivates, containing a hydroxyalkyl moiety on the quaternary amine, such as 1,2-dioleoyl-3-dimethyl-hydroxyethyl ammonium bromide (DORI); 1,2-dioleyloxypropyl-3-dimethyl-hydroxyethyl ammonium bromide (DORIE); 1,2-dioleyloxypropyl-3-dimethyl-hydroxypropyl ammonium bromide (DORIE-HP), 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1,2-dimyristyloxypropyl-3-dimethyl-hydroxylethyl ammonium bromide (DMRIE); 1,2-dipalmityloxypropyl-3-dimethyl-hydroxyethyl ammonium bromide (DPRIE); 1,2-disteryloxypropyl-3-dimethyl-hydroxyethyl ammonium bromide (DSRIE) (e.g., as described in Felgner et al. (1994) J. Biol. Chem. 269:2550-2561, herein incorporated by reference in its entirety). Many of the above-mentioned lipids are available commercially from, e.g., Avanti Polar Lipids, Inc.; Sigma Chemical Co.; Molecular Probes, Inc.; Northern Lipids, Inc.; Roche Molecular Biochemicals; and Promega Corp.

In certain embodiments, the neutral lipids employed (e.g., pre-injected prior to any subcutaneous injections) with the methods, compositions, systems, and kits includes diacylglycerophosphorylcholine wherein the acyl chains are generally at least 12 carbons in length (e.g., 12 . . . 14 . . . 20 . . . 24 . . . or more carbons in length) and may contain one or more cis or trans double bonds. Examples of the compounds include, but are not limited to, distearoyl phosphatidyl choline (DSPC), dimyristoyl phosphatidylcholine (DMPC), dipalmitoyl phosphatidylcholine (DPPC), palmitoyl oleoyl phosphatidylcholine (POPC), palmitoyl stearoyl phosphatidylcholine (PSPC), egg phosphatidylcholine (EPC), hydrogenated or non-hydrogenated soya phosphatidylcholine (HSPC), or sunflower phosphatidylcholine.

In certain embodiments, the neutral lipids include, for example, up to 70 mol diacylglycerophosphorylethanolamine/100 mol phospholipid (e.g., 10/100 mol 25/100 mol . . . 50/100 . . . 70/100 mol). In some embodiments, the diacylglycerophosphorylethanolamine has acyl chains that are generally at least 12 carbons in length (e.g., 12 . . . 14 . . . 20 . . . 24 . . . or more carbons in length), and may contain one or more cis or trans double bonds. Examples of such compounds include, but are not limited to distearoylphosphatidylethanolamine (DSPE), dimyristoylphosphatidylethanolamine (DMPE), dipalmitoylphosphatidylethanolamine (DPPE), palmitoyloleoylphosphatidylethanolamine (POPE), egg phosphatidylethanolamine (EPE), and transphosphatidylated phosphatidylethanolamine (t-EPE), which can be generated from various natural or semisynthetic phosphatidylcholines using phospholipase D.

In one embodiment, the cationic and neutral lipids (e.g., in the pre-treatment solution) are selected from the group consisting of: distearoyl phosphatidyl choline (DSPC); hydrogenated or non-hydrogenated soya phosphatidylcholine (HSPC); distearoylphosphatidylethanolamine (DSPE); egg phosphatidylcholine (EPC); 1,2-Distearoyl-sn-glycero-3-phospho-rac-glycerol (DSPG); dimyristoyl phosphatidylcholine (DMPC); 1,2-Dimyristoyl-sn-glycero-3-phosphoglycerol (DMPG); 1,2-Dipalmitoyl-sn-glycero-3-phosate (DPPA); trimethylammonium propane lipids; DOTIM (1-[2-9(2)-octadecenoylloxy)ethyl]-2-(8(2)-heptadecenyl)-3-(2-hydroxyethyl) midizolinium chloride) lipids; and mixtures of two or more thereof.

In one embodiment, the cationic and neutral lipids are selected from the group consisting of: DOTAP (1,2-dioleoyl-3-trimethylammonium-propane); distearoyl phosphatidyl choline (DSPC); hydrogenated or non-hydrogenated soya phosphatidylcholine (HSPC); distearoylphosphatidylethanolamine (DSPE); egg phosphatidylcholine (EPC); 1,2-Distearoyl-sn-glycero-3-phospho-rac-glycerol (DSPG); dimyristoyl phosphatidylcholine (DMPC); 1,2-Dimyristoyl-sn-glycero-3-phosphoglycerol (DMPG); 1,2-Dipalmitoyl-sn-glycero-3-phosphate (DPPA); trimethylammonium propane lipids; DOTIM (1-[2-9(2)-octadecenoylloxy)ethyl]-2-(8(2)-heptadecenyl)-3-(2-hydroxyethyl) midizolinium chloride) lipids; and mixtures of two or more thereof.

In one embodiment, the lipid comprises DOTAP. In one embodiment, the lipid comprises DMPC.

In some embodiments, the polycationic structure or cationic liposomes comprise lipids selected from the group consisting of: 1,2-dioleoyl-3-trimethylammonium-propane (DOTAP); 1,2-Dimyristoyl-sn-glycero-3-phosphocholine (DMPC); 1,2-dioleoyl-sn-glycero-3-phospho-L-serine (DOPS); and 1-stearoyl-2-oleoyl-sn-glycero-3-phospho-L-serine.

In one embodiment, the polycationic structure or cationic liposome further comprises an anti-inflammatory agent. In one embodiment, the polycationic structure or cationic liposome further comprises dexamethasone or dexamethasone palmitate. In one embodiment, the polycationic structure or cationic liposome further comprises dextrose. In one embodiment, the amount of dextrose is 1-20% (w/w). In one embodiment, the amount of dextrose is 1-10% (w/w).

In one embodiment, the amount of dextrose is 1-5% (w/w). In one embodiment, the amount of dextrose is 5% (w/w).

In some embodiments, prior to the injecting, the subject is treated with dexamethasone fatty acid ester.

In some embodiments, the dexamethasone fatty acid ester employed has the following Formula:

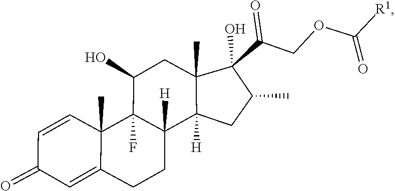

wherein R¹ is C₅-C₂₃ alkyl or C₅-C₂₃ alkenyl.

In one embodiment, the dexamethasone fatty acid ester is dexamethasone palmitate. In one embodiment, the dexamethasone fatty acid ester is dexamethasone acetate.

In one embodiment, the pre-treatment solution comprises water soluble dexamethasone. In one embodiment, the pre-treatment solution comprises dexamethasone-cyclodextrin inclusion complex (see Sigma Aldrich SKU D2915). In one embodiment, the pre-treatment solution comprises dexamethasone sodium phosphate.

In some embodiments, the subject has lung, cardiovascular, and/or multi-organ inflammation. In some embodiments, the subject is on a ventilator. In some embodiments, the composition further comprises a physiologically tolerable buffer or intravenous solution. In other embodiments, the composition further comprises lactated Ringer's solution or saline solution.

In one embodiment, the composition further comprises a physiologically tolerable buffer or intravenous solution.

In some embodiments, prior to the administration of the nucleic acid encoding the at least one protein or the BANEM, the method further comprises administering a solution to the subject comprising liposomes comprising the polycationic structures, wherein the liposomes further comprising one or more macrophage targeting moieties selected from the group consisting of: mannose moieties, maleimide moieties, a folate receptor ligand, folate, folate receptor antibody or fragment thereof, formyl peptide receptor ligands, N-formyl-Met-Leu-Phe, tetrapeptide Thr-Lys-Pro-Arg, galactose, lactobionic acid, a lipid bi-layer integrating peptide and/or a target peptide.

In some embodiments, a fatty acid ester of dexamethasone is employed. In some embodiments, the fatty acid ester is a C₆-C₂₄ fatty acid ester, such as hexanoate (caproate), heptanoate (enanthate), octanoate (caprylate), nonanoate (pelargonate), decanoate (caprate), undecanoate, dodecanoate (laurate), tetradecanoate (myristate), octadecenoate (stearate), icosanoate (arachidate), docosanoate (behenate), and tetracosanoate (lignocerate). Accordingly, in some embodiments, the compound is selected from dexamethasone caproate, dexamethasone enanthate, dexamethasone caprylate, dexamethasone pelargonate, dexamethasone caprate, dexamethasone undecanoate, dexamethasone laurate, dexamethasone myristate, dexamethasone palmitate, dexamethasone stearate, dexamethasone arachidate, dexamethasone behenate, and dexamethasone lignocerate.

Therapeutic Use

In some embodiment, the method is used for treating, managing or preventing a disease, a condition or a symptom in a subject.

In some embodiments, the method is used for treating, managing or preventing lipid storage disorders. In one embodiment, the method is used for treating, managing or preventing Fabry disease. In one embodiment, the method is used for treating, managing or preventing Gaucher disease. In one embodiment, the method is used for treating, managing or preventing multiple sulfatase deficiency. In one embodiment, the method is used for treating, managing or preventing Farber's lipogranulomatosis. In one embodiment, the method is used for treating, managing or preventing Niemann-Pick disease. In one embodiment, the method is used for treating, managing or preventing Wolman disease. In one embodiment, the method is used for treating, managing or preventing lysosomal storage disorders. In one embodiment, the method is used for treating, managing or preventing Galactosialidosis. In one embodiment, the method is used for treating, managing or preventing Schindler disease. In one embodiment, the method is used for treating Fucosidosis. In one embodiment, the method is used for treating, managing or preventing Pompe disease. In some embodiments, the method is used for treating, managing or preventing Tay-Sachs disease. In some embodiments, the method is used for treating, managing or preventing Sandhoff's disease. In some embodiments, the method is used for treating, managing or preventing metachromatic leukodystrophy. In some embodiments, the method is used for treating, managing or preventing cholesterol ester storage disease. In some embodiments, the method is used for treating, managing or preventing type 2 diabetes. In some embodiments, the method is used for treating, managing or preventing non-alcoholic or alcoholic fatty liver disease. In some embodiments, the method is used for treating, managing or preventing cancers.

In one embodiment, the method is used for treating, managing or preventing infectious diseases caused by a pathogen. In some embodiments the infectious pathogen is selected from virus, bacteria, fungi or parasite. In one embodiment, the method is used for treating, managing or preventing viral infection. In one embodiment, the method is used for treating, managing or preventing SARS-Cov-2 infection. In one embodiment, the method is used for treating, managing or preventing influenza infection. In one embodiment, the method is used for treating, managing or preventing influenza A infection. In one embodiment, the method is used for treating, managing or preventing influenza B infection.

In one embodiment, the method is used for treating, managing or preventing a genetic disease. In some embodiments, the method is used for treating, managing or preventing a cancer.

In other embodiment, the method is used for treating, managing or preventing conditions as listed in column F of Table 4.

In one embodiment, the subject is a human or livestock. In one embodiment, the subject is a human.

In one embodiment, the subject is overweight by BMI standards (e.g., a BMI of 25 . . . 27 . . . 29, 30 . . . 35 . . . 40 or more) and/or is overweight or obese (e.g., clinically obese). In some embodiments, the injecting (e.g., with a syringe) in a), b), or c) ii) is into a fat pad of the subject (e.g., a location where millions of pre-adipocytes and/or adipocytes are located).

In one embodiment, the subject is overweight by BMI standards and/or is clinically obese. In one embodiment, described here in a method of treatment a subject that is overweight.

In one embodiment, the subject is overweight by BMI standards. In one embodiment, the subject has a BMI of at least 25. In one embodiment, the subject has a BMI of at least 26. In one embodiment, the subject has a BMI of at least 27. In one embodiment, the subject has a BMI of at least 28. In one embodiment, the subject has a BMI of at least 29. In one embodiment, the subject has a BMI of at least 30. In one embodiment, the subject has a BMI of at least 32. In one embodiment, the subject has a BMI of at least 34. In one embodiment, the subject has a BMI of at least 36. In one embodiment, the subject has a BMI of at least 38. In one embodiment, the subject has a BMI of at least 40. In one embodiment, the subject is clinically obese. In one embodiment, the subject is both overweight by BMI standards and clinically obese.

In one embodiment, the subject has a normal weight by BMI standards. In one embodiment, the subject has a BMI between 18.5 to 25. In one embodiment, the subject has a BMI of 19, 20, 21, 22, 23, 24 or 25. In one embodiment, described here in a method of treatment a subject that is has normal weight.

In one embodiment, the subject is underweight by BMI standards. In one embodiment, the subject has a BMI lower than 18.5. In one embodiment, described here in a method of treatment a subject that is underweight.

Effect of Administration

In some embodiments, upon subcutaneous administration of the composition comprising the nucleic acid encoding at least one protein or other BANEM to a subject, the encoded at least one protein and/or BANEM is expressed in the subject at a therapeutic level for a sustained period of time. In some embodiments, upon administration of the composition comprising the ex-vivo transfected adipocyte and/or pre-adipocyte, the encoded at least one protein and/or BANEM is expressed in the subject at a therapeutic level for a sustained period of time. In some embodiments, the at least one protein and/or BANEM is expressed in the subject at a level sufficient to induce a therapeutic effect in the subject.

In some embodiments, the encoded protein is expressed in the blood of the subject at a level of at least 50 μg/ml. In one embodiment, the protein is expressed in the blood of the subject at a level of at least 50 ng/ml. In one embodiment, the protein is expressed in the blood of the subject at a level of at least 100 ng/ml.

In one embodiment, the protein is expressed in the blood of the subject at a level of at least 200 ng/ml. In one embodiment, the protein is expressed in the blood of the subject at a level of at least 300 ng/ml. In one embodiment, the protein is expressed in the blood of the subject at a level of at least 400 ng/ml. In one embodiment, the protein is expressed in the blood of the subject at a level of at least 500 ng/ml. In one embodiment, the protein is expressed in the blood of the subject at a level of at least 600 ng/ml. In one embodiment, the protein is expressed in the blood of the subject at a level of at least 700 ng/ml. In one embodiment, the protein is expressed in the blood of the subject at a level of at least 800 ng/ml. In one embodiment, the protein is expressed in the blood of the subject at a level of at least 900 ng/ml. In one embodiment, the protein is expressed in the blood of the subject at a level of at least 1000 ng/ml. In one embodiment, the protein is expressed in the blood of the subject at a level of at least 1 μg/ml. In one embodiment, the protein is expressed in the blood of the subject at a level of at least 10 μg/ml. In one embodiment, the protein is expressed in the blood of the subject at a level of at least 50 μg/ml. In one embodiment, the protein is expressed in the blood of the subject at a level of at least 100 μg/ml. In one embodiment, the protein is expressed in the blood of the subject at a level of at least 150 μg/ml. In one embodiment, the protein is expressed in the blood of the subject at a level of at least 500 μg/ml. In one embodiment, the protein is expressed in the blood of the subject at a level of at least 800 μg/ml. In one embodiment, the protein is expressed in the blood of the subject at a level of at least 900 μg/ml. In one embodiment, the protein is expressed in the blood of the subject at a level of at least 1 mg/ml. In one embodiment, the protein is expressed in the blood of the subject at a level of at least 2 mg/ml. In one embodiment, the protein is expressed in the blood of the subject at a level of at least 3 mg/ml. In one embodiment, the protein is expressed in the blood of the subject at a level of at least 4 mg/ml. In one embodiment, the protein is expressed in the blood of the subject at a level of at least 5 mg/ml. In one embodiment, the protein is expressed in the blood of the subject at a level of at least 10 mg/ml. In some embodiments, the protein expression level is measured from a blood sample, such as a whole blood sample, a serum sample or a plasma sample taken from the subject.

In some embodiments, the encoded protein and/or BANEM is expressed in the subject at a therapeutic level for a sustained period of time. In specific embodiments, the encoded protein and/or BANEM is expressed in the subject at a therapeutic level for at least 50, at least 60, at least 70, at least 80, at least 90, at least 100 days, at least 120 days or at least 150 days after receiving the last dose of administration subcutaneously. In some embodiments, the subject is a mouse or a rat. In some embodiments, the encoded protein and/or BANEM is expressed in the subject at a therapeutic level for a period of time equivalent to at least one month, two months, four months, six months, eight months, ten months, twelve months, 2 years, 5 years, a decade or more in human after receiving the last dose of administration subcutaneously.

In one embodiment, the expression level of protein and/or BANEM is maintained in the subject for at least one month without any further treatment. In one embodiment, the expression level is maintained for at least two months without any further treatment. In one embodiment, the expression level is maintained for at least four months without any further treatment. In one embodiment, the expression level is maintained for at least six months without any further treatment. In one embodiment, the expression level is maintained for at least eight months without any further treatment. In one embodiment, the expression level is maintained for at least ten months without any further treatment. In one embodiment, the expression level is maintained for at least twelve months without any further treatment. In one embodiment, the expression level is maintained for at least 2 years without any further treatment. In one embodiment, the expression level is maintained for at least 4 years without any further treatment. In one embodiment, the expression level is maintained for at least 6 years without any further treatment. In one embodiment, the expression level is maintained for at least 8 years without any further treatment. In one embodiment, the expression level is maintained for at least 10 years without any further treatment.

In one embodiment, the expression of protein and/or biologically active nucleic acid is detectable in the subject for at least one month without any further treatment. In one embodiment, the expression is detectable for at least two months without any further treatment. In one embodiment, the expression is detectable for at least four months without any further treatment. In one embodiment, the expression is detectable for at least six months without any further treatment. In one embodiment, the expression is detectable for at least eight months without any further treatment. In one embodiment, the expression is detectable for at least ten months without any further treatment. In one embodiment, the expression is detectable for at least twelve months without any further treatment. In one embodiment, the expression is detectable for at least 2 years without any further treatment. In one embodiment, the expression is detectable for at least 4 years without any further treatment. In one embodiment, the expression is detectable for at least 6 years without any further treatment. In one embodiment, the expression is detectable for at least 8 years without any further treatment. In one embodiment, the expression is detectable for at least 10 years without any further treatment.

In some embodiments, upon administration of the composition comprising the nucleic acid encoding at least one protein or other BANEM in a subject, a plurality of in-vivo transfected pre-adipocytes and/or adipocytes are generated in the subcutaneous region(s) receiving the injection or implantation.

In one embodiment, in-vivo transfected pre-adipocytes and/or adipocytes are enlarged such that, on average, they contain at least 60 μg of lipid (e.g., at least 60 . . . 70 . . . 90 . . . 100 . . . 110 ug . . . etc.). In further embodiments, the plurality of in-vivo transfected pre-adipocytes and/or adipocytes are enlarged such that, on average, they contain at least 80 ug of lipid (e.g., at least 90 . . . 100 . . . 110 ug . . . etc.). In further embodiments, the plurality of in-vivo transfected pre-adipocytes and/or adipocytes are enlarged such that, on average, they have a diameter of at least 70 μm (e.g., at least 70 . . . 80 . . . 90 . . . 100 . . . 110 μm). In additional embodiments, the plurality of in-vivo transfected pre-adipocytes and/or adipocytes are enlarged such that, on average, they have a diameter of at least 90 μm (e.g., at least 90 . . . 100 . . . 110 . . . 120 μm).

In certain embodiments, the transfected and enlarged adipocytes contain, on average, at least 60 μg of lipid (e.g., 60 . . . 70 . . . 80 . . . 90 . . . ug). In some embodiments, the plurality transfected pre-adipocytes and/or adipocytes contain, on average, at least 80 μg of lipid. In other embodiments, the transfected pre-adipocytes and/or adipocytes have, on average, a diameter of at least 70 μm. In additional embodiments, the transfected pre-adipocytes and/or adipocytes have, on average, a diameter of at least 90 μm. In additional embodiments, the transfected pre-adipocytes and/or adipocytes are derived from one or more subcutaneous regions of a subject.

In one embodiment, the plurality of in-vivo transfected pre-adipocytes and/or adipocytes are enlarged such that, on average, they contain at least 60 μg of lipid. In one embodiment, they contain at least 65 μg of lipid. In one embodiment, they contain at least 70 μg of lipid. In one embodiment, they contain at least 70 μg of lipid. In one embodiment, they contain at least 75 μg of lipid. In one embodiment, they contain at least 80 μg of lipid. In one embodiment, they contain at least 85 μg of lipid. In one embodiment, they contain at least 90 μg of lipid. In one embodiment, they contain at least 95 μg of lipid. In one embodiment, they contain at least 100 μg of lipid. In one embodiment, they contain at least 110 μg of lipid. In one embodiment, they contain at least 120 μg of lipid. In one embodiment, they contain at least 130 μg of lipid. In one embodiment, they contain at least 150 μg of lipid. In one embodiment, they contain at least 200 μg of lipid.

In one embodiment, the plurality of in-vivo transfected pre-adipocytes and/or adipocytes are enlarged such that, on average, they have a diameter of at least 70 μm. In one embodiment, they have a diameter of at least 75 μm. In one embodiment, they have a diameter of at least 80 μm. In one embodiment, they have a diameter of at least 85 μm. In one embodiment, they have a diameter of at least 90 μm. In one embodiment, they have a diameter of at least 95 μm. In one embodiment, they have a diameter of at least 100 μm. In one embodiment, they have a diameter of at least 120 μm. In one embodiment, they have a diameter of at least 150 μm. In one embodiment, they have a diameter of at least 200 μm. In one embodiment, they have a diameter of at least 250 μm.

In one embodiment, the plurality of in-vivo transfected pre-adipocytes and/or adipocytes are enlarged such that, on average, they have a diameter of at least 90 μm.

In one embodiment, the in-vivo transfected pre-adipocytes and/or adipocytes have normal sizes.

In specific embodiments, the encoded protein comprises a therapeutic antibody or antigen-binding portion thereof targeting an pathogenic antigen (e.g., a anti-SARS-CoV-2 antibody), and the expression of such antibody in the subject is at an expression level sufficient to reduce: i) the load of the pathogen in the subject, and/or ii) at least one symptom in the subject caused by the infectious antigen.

In one embodiment, the expression level of the therapeutic antibody in the subject is greater than 1 Unit/mL. In one embodiment, the expression level is greater than 10 Unit/mL. In one embodiment, the expression level is greater than 10 Unit/mL. In one embodiment, the expression level is greater than 100 Unit/mL. In one embodiment, the expression level is greater than 200 Unit/mL. In one embodiment, the expression level is greater than 500 Unit/mL. In one embodiment, the expression level is greater than 1000 Unit/mL. In one embodiment, the expression level is greater than 2000 Unit/mL.

In one embodiment, the therapeutic antibody reduces the pathogen load by 10%, 20%, 30%, 40%, 50%, 60%, 70%, 80%, 90%, 95% or 100% in the subject. In one embodiment, the therapeutic antibody reduces the symptom in the subject caused by the pathogenic infection. In one embodiment, the therapeutic antibody reduces risk of hospitalization or death in the subject caused by the pathogen infection.

In some embodiments, the pathogen is a coronavirus (e.g., the SARS-Cov-2 virus). In one embodiment, the antibody binds to one or more epitopes of the S protein of the coronavirus and inhibits or reduces one or more S protein function or activity. In one embodiment, binding of the S protein to its cellular receptor is reduced or inhibited. In one embodiment, binding of the coronavirus S protein to angiotensin-converting enzyme 2 (ACE2), and/or sugar on the host cell surface is reduced or inhibited. In one embodiment, attachment of the coronavirus with host cells in the subject is reduced or inhibited. In one embodiment, infection of host cells in the subject by the coronavirus is reduced or inhibited. In some embodiments, the antibody reduces the S protein function or activity by 10%, 20%, 30%, 40%, 50%, 60%, 70%, 80%, 90%, 95% or 100%. In one embodiment, the antibody against the coronavirus or cells infected by the coronavirus is produced in the subject. In one embodiment, the antibody specifically binds to one or more epitopes of the N protein of the coronavirus and inhibits or reduces one or more N protein function or activity. In one embodiment, binding of the coronavirus N protein to reproduced viral genomic sequences is reduced or inhibited. In one embodiment, embodiments, reproduction of viable progenies of the coronavirus is reduced or inhibited. In one embodiment, the antibody reduces the function or activity of the virus by 10%, 20%, 30%, 40%, 50%, 60%, 70%, 80%, 90%, 95% or 100%.

### Systems and Kits

Also provided here are systems and kits comprising: a) a plurality of transfected pre-adipocytes and/or adipocytes (e.g., which are enlarged), wherein each of the plurality of transfected pre-adipocytes and/or adipocytes comprises an exogenous nucleic acid sequence, or a vector containing the nucleic acid sequence, wherein the nucleic acid sequence encodes at least one protein or at least one biologically active nucleic acid molecule, and b) a first container, wherein the plurality of transfected pre-adipocytes and/or adipocytes (e.g., which are enlarged) are present in the first container.

In one embodiment, the system comprises:
a) a plurality of transfected and enlarged adipocytes or pre-adipocytes, wherein each of the plurality of transfected and enlarged adipocytes or pre-adipocytes comprises an exogenous nucleic acid sequence, or a vector containing the nucleic acid sequence, wherein the nucleic acid sequence encodes at least one protein or at least one biologically active nucleic acid molecule, and
b) a first container, wherein the plurality of transfected and enlarged adipocytes or pre-adipocytes are present in the first container.

In one embodiment, the first container comprises a syringe configured for injecting the plurality of transfected and enlarged adipocytes or pre-adipocytes into a subject subcutaneously. In one embodiment, the adipocytes or pre-adipocytes are enlarged.

In some embodiments, the systems or kit further comprises: c) a solution comprising at least one of the following: i) cationic liposomes, ii) neutral liposomes, iii) dexamethasone, and iv) dexamethasone palmitate. In one embodiment, the system further comprises cationic liposomes. In one embodiment, the system further comprises neutral liposomes. In one embodiment, the system further comprises dexamethasone. In one embodiment, the system further comprises dexamethasone palmitate. In one embodiment, the system further comprises both cationic liposomes and dexamethasone. In one embodiment, the system further comprises both cationic liposomes and dexamethasone palmitate. In one embodiment, the system further comprises both neutral liposomes and dexamethasone. In one embodiment, the system further comprises both neutral liposomes and dexamethasone palmitate. In one embodiment, the system further comprises cationic liposomes, dexamethasone and dexamethasone palmitate. In one embodiment, the system further comprises neutral liposomes, dexamethasone and dexamethasone palmitate. In one embodiment, the system further comprises cationic liposome, neutral liposomes, and dexamethasone palmitate.

In embodiment, the systems or kit is used for treating diseases. In embodiment, the systems or kit is used for treating genetic diseases. In embodiment, the systems or kit is used for treating infectious diseases. In embodiment, the systems or kit is used for treating infectious diseases caused by virus, particularly coronavirus. In embodiment, the systems or kit is used for treating diseases caused SARS-Cov-2. In embodiment, the systems or kit is used for treating diseases caused by influenza, particularly influenza A or influenza B. In embodiment, the systems or kit is used for treating lipid storage disorders. In embodiment, the systems or kit is used for treating lysosomal storage disorders. In embodiment, the systems or kit is used for treating Galactosialidosis. In embodiment, the systems or kit is used for treating Fabry disease. In embodiment, the systems or kit is used for treating Gaucher disease. In embodiment, the systems or kit is used for treating multiple sulfatase deficiency. In embodiment, the systems or kit is used for treating Farber's lipogranulomatosis. In embodiment, the systems or kit is used for treating Niemann-Pick disease. In embodiment, the systems or kit is used for treating Wolman disease. In embodiment, the systems or kit is used for treating Schindler disease. In embodiment, the systems or kit is used for treating Fucosidosis. In embodiment, the systems or kit is used for treating Pompe disease.

In one embodiment, the plurality transfected and enlarged adipocytes or pre-adipocytes contain, on average, at least 60 μg of lipid. In one embodiment, the plurality transfected and enlarged adipocytes or pre-adipocytes contain, on average, at least 70 μg of lipid. In one embodiment, the plurality transfected and enlarged adipocytes or pre-adipocytes contain, on average, at least 80 μg of lipid. In one embodiment, the plurality transfected and enlarged adipocytes or pre-adipocytes contain, on average, at least 90 μg of lipid. In one embodiment, the plurality transfected and enlarged adipocytes or pre-adipocytes contain, on average, at least 100 μg of lipid. In one embodiment, the plurality transfected and enlarged adipocytes or pre-adipocytes contain, on average, at least 120 μg of lipid. In one embodiment, the plurality transfected and enlarged adipocytes or pre-adipocytes contain, on average, at least 140 μg of lipid. In one embodiment, the plurality transfected and enlarged adipocytes or pre-adipocytes contain, on average, at least 160 μg of lipid. In one embodiment, the plurality transfected and enlarged adipocytes or pre-adipocytes contain, on average, at least 180 μg of lipid. In one embodiment, the plurality transfected and enlarged adipocytes or pre-adipocytes contain, on average, at least 200 μg of lipid.

In one embodiment, the transfected and enlarged adipocytes or pre-adipocytes have, on average, a diameter of at least 70 μm. In one embodiment, the transfected and enlarged adipocytes or pre-adipocytes have, on average, a diameter of at least 80 μm. In one embodiment, the transfected and enlarged adipocytes or pre-adipocytes have, on average, a diameter of at least 90 μm. In one embodiment, the transfected and enlarged adipocytes or pre-adipocytes have, on average, a diameter of at least 100 μm. In one embodiment, the transfected and enlarged adipocytes or pre-adipocytes have, on average, a diameter of at least 120 μm. In one embodiment, the transfected and enlarged adipocytes or pre-adipocytes have, on average, a diameter of at least 140 μm. In one embodiment, the transfected and enlarged adipocytes or pre-adipocytes have, on average, a diameter of at least 160 μm. In one embodiment, the transfected and enlarged adipocytes or pre-adipocytes have, on average, a diameter of at least 180 μm. In one embodiment, the transfected and enlarged adipocytes or pre-adipocytes have, on average, a diameter of at least 200 μm. In one embodiment, the transfected and enlarged adipocytes or pre-adipocytes have, on average, a diameter of at least 250 μm.

In one embodiment, the transfected and enlarged adipocytes or pre-adipocytes are derived from one or more subcutaneous regions of a subject.

TABLE 1

| | DNA Codons | DNA Codons |
|---|---|---|
| Amino Acid | Containing CpG | Lacking CpG |
| Serine (Ser or S) | TCG | TCT, TCC, TCA, |
| | | AGT, AGC |
| Proline (Pro or P) | CCG | CCT, CCC, CCA, |
| Threonine (Thr or T) | ACG | ACA, ACT, ACC |
| Alanine (Ala or A) | GCG | GCT, GCC, GCA |
| Arginine (Arg or R) | CGT, CGC, | AGA, AGG |
| | CGA, CGG | |

In addition, within the coding region, the interface between triplets should be taken into consideration. For example, if an amino acid triplet ends in a C-nucleotide which is then followed by an amino acid triplet which can start only with a G-nucleotide (e.g., Valine, Glycine, Glutamic Acid, Alanine, Aspartic Acid), then the triplet for the first amino acid triplet is changed to one which does not end in a C-nucleotide. Methods for making CpG free sequences are shown, for example, in U.S. Pat. No. 7,244,609, which is herein incorporated herein by reference. A commercial service provided by INVIVOGEN is also available to produce CpG free (or reduced) nucleic acid sequences/vectors (plasmids). A commercial service provided by ThermoScientific produces CpG free nucleotide sequences.

Provided below in Table 2 are exemplary promoters and enhancers that may be used in the vectors described herein. Such promoters, and other promoters known in the art, may be used alone or with any of the enhancers, or enhancers, known in the art. Additionally, when multiple proteins or biologically active nucleic acid molecules (e.g., two, three, four, or more) are expressed from the same vector, the same or different promoters may be used in conjunction with the subject nucleic acid sequence. In some embodiments, a promoter selected from the following list is employed to control the expression levels of the protein or nucleic acid: FerL, FerH, Grp78, hREG1B, and cBOX1. Such promoter can be used, for example, to control production of a protein (e.g., HGH) protein production over a broad temporal range (e.g., without the use of any other modifications including Gene switches).

TABLE 2

| Promoter | Enhancer |
|---|---|
| CMV | human CMV |
| EF1α | mouse CMV |
| Ferritin (Heavy/Light) Chain | SV40 |
| GRP94 | Ubc |
| U1 | AP1 |
| UbC | hr3 |
| Beta Actin | IE2 |
| PGK1 | IE6 |
| GRP78 | E2-RS |
| CAG | MEF2 |
| SV40 | C/EBP |
| TRE | |
| FerL | |
| FerH | |
| Grp78 | |
| hREG1B | |
| cBOX1 | HNF-1 |

In some embodiments, compositions and systems herein are provided and/or administered (e.g., injected subcutaneously) in doses selected to elicit a therapeutic and/or prophylactic effect in an appropriate subject (e.g., mouse, human, etc.). In some embodiments, a therapeutic dose is provided. In some embodiments, a prophylactic dose is provided. Dosing and administration regimes are tailored by the clinician, or others skilled in the pharmacological arts, based upon well-known pharmacological and therapeutic/prophylactic considerations including, but not limited to, the desired level of pharmacologic effect, the practical level of pharmacologic effect obtainable, toxicity. Generally, it is advisable to follow well-known pharmacological principles for administrating pharmaceutical agents (e.g., it is generally advisable to not change dosages by more than 50% at time and no more than every 3-4 agent half-lives). For compositions that have relatively little or no dose-related toxicity considerations, and where maximum efficacy is desired, doses in excess of the average required dose are not uncommon. This approach to dosing is commonly referred to as the “maximal dose” strategy. In certain embodiments, a dose (e.g., therapeutic of prophylactic) of nucleic acid or vector is about 0.01 mg/kg to about 200 mg/kg (e.g., 0.01 mg/kg, 0.02 mg/kg, 0.05 mg/kg, 0.1 mg/kg, 0.2 mg/kg, 0.5 mg/kg, 1.0 mg/kg, 2.0 mg/kg, 5.0 mg/kg, 10 mg/kg, 20 mg/kg, 50 mg/kg, 100 mg/kg, 200 mg/kg, or any ranges therebetween (e.g., 5.0 mg/kg to 100 mg/kg)). In some embodiments, a subject is between 0.1 kg (e.g., mouse) and 150 kg (e.g., human), for example, 0.1 kg, 0.2 kg, 0.5 kg, 1.0 kg, 2.0 kg, 5.0 kg, 10 kg, 20 kg, 50 kg, 100 kg, 200 kg, or any ranges therebetween (e.g., 40-125 kg). In some embodiments, a dose of nucleic acid, or vector containing the nucleic acid, comprises between 0.001 mg and 40,000 mg (e.g., 0.001 mg, 0.002 mg, 0.005 mg, 0.01 mg, 0.02 mg, 0.05 mg, 0.1 kg, 0.2 mg, 0.5 mg, 1.0 mg, 2.0 mg, 5.0 mg, 10 mg, 20 mg, 50 mg, 100 mg, 200 mg, 500 mg, 1,000 mg, 2,000 mg, 5,000 mg, 10,000 mg, 20,000 mg, 40,000 mg, or ranges therebetween.

In certain embodiments, a target peptide is used with the cationic or neutral liposomes in the pre-treating solutions herein. Exemplary target peptides are shown in Table 3 below. In table 3, “[n]” prefix indicates the N-terminus and a “[c]” suffix indicates the C-terminus; sequences lacking either are found in the middle of the protein.

TABLE 3A

| Target | Sequence | Source protein or organism |
|---|---|---|
| nucleus (NLS) | PKKKRKV (SEQ ID NO: 1) | SV40 large Tantigen (P03070) |
| Out of nucleus (NES) | IDMLIDLGLDLSD (SEQ ID NO: 2) | HSV transcriptional regulator |
| | | IE63 P10238 |
| ER, secretion (signal | [n]MMSFVSLLLVGILFWATEAEQLTKCE | Lactalbumin (P09462) |
| peptide) | VFQ (SEQ ID NO: 3) | |
| ER, retention (KDEL) | KDEL[c] (SEQ ID NO: 4) | |
| Mitochondrial matrix | [n]MLSLRQSIRFFKPATRTLCSSRYLL | S. cerevisiae COX4 (P04037) |
| | (SEQ ID NO: 5) | |
| Plastid | [n]MVAMAMASLQSSMSSLSLSSNSFLGQ | Pisum sativum RPL24 (P11893) |
| | PLSPITLSPFLQG (SEQ ID NO: 6) | |
| Folded secretion (Tat) | (S/T)RRXFLK (SEQ ID NO: 7)(“X” refers | Near the N terminus^{[6]} |
| | to any naturally occurring amino acid) | |
| peroxisome (PTS1) | SKL[c] (SEQ ID NO: 8) | |
| peroxisome (PTS2) | [c]XXXXRLXXXXXHL (SEQ ID NO: 9) | |
| | (“X” refers to any naturally occurring amino | |
| | acid) | |

In one embodiment, the vector as used herein comprises at least one sequence as listed in Table 3B below.

In one embodiment, the vector as used herein comprises an enhancer. In one embodiment, the enhancer comprises a sequence as set forth in SEQ ID NO: 16 or SEQ ID NO: 17.

In one embodiment, the vector as used herein comprises a promoter. In one embodiment, the promoter comprises a sequence as set forth in SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26.

In one embodiment, the vector as used herein encodes a linker. In one embodiment, the linker is 1× linker. In one embodiment, the linker is a 2× linker. In one embodiment, the linker comprises a sequence as set forth in SEQ ID NO: 27 or SEQ ID NO: 28.

In one embodiment, the vector as used herein comprises a matrix-attachment region (MAR). In one embodiment, the MAR comprises a sequence as set forth in SEQ ID NO: 29, SEQ ID NO: 30 or SEQ ID NO: 31.

In one embodiment, the vector as used herein comprises a super-enhancer. In one embodiment, the super-enhancer comprises a sequence as set forth in SEQ ID NO: 32, SEQ ID NO: 33, SEQ ID NO: 34,

SEQ ID NO: 35, SEQ ID NO: 36 or SEQ ID NO: 37.

In one embodiment, the vector as used herein comprises a long-term repeat (LTR) within intron. In one embodiment, the LTR comprises a sequence as set forth in SEQ ID NO: 38, SEQ ID NO: 39, SEQ ID NO: 40 or SEQ ID NO: 41.

In one embodiment, the vector as used herein comprises a nucleus localization sequence-microtubule-associated sequence (NLS-MTAS). In one embodiment, NLS-MTAS further comprises a hydrophilic linker and modified neck domain peptide (NDP). In one embodiment, the NLS-MTAS comprises a sequence as set forth in SEQ ID NO: 42, SEQ ID NO: 43, SEQ ID NO: 44 or SEQ ID NO: 45.

TABLE 3B

| | |
|---|---|
| SEQUENCE | |
| NAME | |
| (SEQ ID NO) | AMINO ACID OR NUCLEIC ACID SEQUENCE |

Enhancer

| | |
|---|---|
| M-CmvEnh | GAGTCAATGGGAAAAACCCATTGGAGCCAAGTACACTGACTCAATAG |
| (SEQ ID NO: 16) | GGACTTTCCATTGGGTTTTGCCCAGTACATAAGGTCAATAGGGGGTG |
| | AGTCAACAGGAAAGTCCCATTGGAGCCAAGTACATTGAGTCAATAGG |
| | GACTTTCCAATGGGTTTTGCCCAGTACATAAGGTCAATGGGAGGTAA |
| | GCCAATGGGTTTTTCCCATTACTGACATGTATACTGAGTCATTAGGGA |
| | CTTTCCAATGGGTTTTGCCCAGTACATAAGGTCAATAGGGGTGAATC |
| | AACAGGAAAGTCCCATTGGAGCCAAGTACACTGAGTCAATAGGGACT |
| | TTCCATTGGGTTTTGCCCAGTACAAAAGGTCAATAGGGGGTGAGTCA |
| | ATGGGTTTTTCCCATTATTGGCACATACATAAGGTCAATAGGGGTG |
| H-CmvEnh | GTTACATAACTTATGGTAAATGGCCTGCCTGGCTGACTGCCCAATGAC |
| (SEQ ID NO: 17) | CCCTGCCCAATGATGTCAATAATGATGTATGTTCCCATGTAATGCCAA |
| | TAGGGACTTTCCATTGATGTCAATGGGTGGAGTATTTATGGTAACTGC |
| | CCACTTGGCAGTACATCAAGTGTATCATATGCCAAGTATGCCCCCTAT |
| | TGATGTCAATGATGGTAAATGGCCTGCCTGGCATTATGCCCAGTACAT |
| | GACCTTATGGGACTTTCCTACTTGGCAGTACATCTATGTATTAGTCAT |
| | TGCTATTA |

Promoter

| | |
|---|---|
| H-CMV | CCATGGTGATGGGTTTTGGCAGTACATCAATGGGTGTGGATAGTGGT |
| (SEQ ID NO: 18) | TTGACCCATGGGGATTTCCAAGTCTCCACCCCATTGATGCCAATGGGA |
| | GTTTGTTTTGGCACCAAAATCAATGGGACTTTCCAAAATGTTGTAACA |
| | ACTCTGCCCCATTGATGGAAATGGGTGGTAGGTGTGTGTGGTGGGAG |
| | GTCTATATAAGCAGAGCTTGTTTAGTGAACTGGATGCACCTACTA |
| H-FerL from | TCAGGGCCCCAACCCCCCCAAGCCCCCATTTCACAACATGCTGGTGCT |
| pVIVO2, 281 bp: 24 | ACAGGTGTGTGACTTCCCCTTGCTTTGGGGTGGGGGGCTGAGACTCCT |
| TG | ATGTGCTCTGGATTGGTCAGGCATGGCCTTTGGCCCTGCCTCCTGCCA |
| (SEQ ID NO: 19) | CTGCAGATTGGCTGCTAGGCCTCCCTGAGTGCCCTGCCTCTGAGGGCT |
| | GGTGCACCATAAAAGAAGCTGCCCTAGCCATGTCCCCTTGCAGTTTG |
| | GTGGTCCTGTGGGTCTGTCTCAAGCTTGCTGCCAGAACACAG |
| H-FerH from | TTCTGCCAGAGTGTGTGAGGGCCTCCAGTGGCTGCCCCTCCCCCACAG |
| pVIVO2, 182 bp: 26 | CAGGGGTGGGGTCCTGTGCCCACTGGAAGGAGTGGGCTTGGGGTGGG |
| TG | TGGTGCTGATTGGCTGGGGTGGGCCTGATGCTGATGTGGCTATAAGA |
| (SEQ ID NO: 20) | GACCACAAGTGACCTGCAGGGCCAGATGTTCTTTGCTGAA |
| Grp78 from | AGTTACTGGTGGAAATGGTCTTGGGTTGAGAGGTCACCTGAGGGACA |
| pVIVO1, 547 bp: 59 | GGCAGCTGCTGAACCAATAGGACTGGTGCACAGGGTGGATGCTGCCC |
| TG | CTCATTGGTGGCTGTTGAGAGTGACCAAGAGCCAATGAGTCAGCCTG |
| (SEQ ID NO: 21) | GGGGGTGTAGCAGTGATGTAAGTTGTGGAGGAGGCTGCTTTGAATTG |
| | GCAGTGGCCAGCTTGGTGGCATGGACCAATCAGTGTCCTCCAATGAG |
| | GAGTGCCTTTGCCAATTGGAGGCCTCCATGATGGGGCTGGGGGGAGG |
| | GTATATAAGCTGAGTTGGTGGTGGTGTGCTCCACATGGGCTGAGACC |
| | ACAGTGATGGGAGTGTCTGCCTCTGTGGGGCTGAGAGGTAAGTGCTG |
| | TGGCCTGCCCTTTCCAGGCCAACTTGGAGCCTGTCTTGTGGCTCTGCC |
| | TGATTGGGGGCTCCTGTTGCCCTCAGATTGGTTGGAATGCTGTTGTGC |
| | TCTGGGACTACAAGCCTGTTGCTGGGCCTGGAGACTGCTGAAGGACT |
| | GCTGAGCACTGTCCTCAGTGCTGGCAcc |
| M-FABP2 (Murine | GTGCAGTCGACACTGTCTCAAAAACCAAACCAAACCAAAACCCAACC |
| Fatty Acid Binding | CAAACCAAACAAAGCCAAACAACAACAACAAAAACAAAAAAACCCA |
| Protein 2) | CCAAAAAAACCAAGGAAACAAAACAACCAAAAATCAAAAAACTAGG |
| (SEQ ID NO: 22) | CTACTTTAAAATGTCATTATTTATTTTGTTAAAATTCCTGAGATAAAC |
| | ACTATTCTAACAAAAGAGCCATTAAGACTAAGAATCTCTAAGATAGT |
| | TTTTATGTTCTCAAATTCAGAAGAACTAAACACATTATTGCAGTATTA |
| | ATAAAATAAAAACTCAAGATAAGAAGGTCAAATGTGTCCAAGATAAT |
| | TGTCTCCTCCACAATGAGGCAAATCCATAAGGAATAATGGGGGGAAG |
| | TTCAATGCATTAGCTTTTGACAGTCAAAACAGGAACCTTTAAAATACT |
| | CTGTTCATGGTTAAAAATAATTTGTACTCTAAGTCCAGTGATCATTGC |
| | CAGGGAGAACCAAAGTTGAGAAATTTCTATTAAAAACATGACTCAGA |
| | GGAAAACATACAGGGTCTGGTCATGAAGGAAATGATCTGGCCCCCAT |
| | TGGTCACTCCTACAGTCACATGGTCAGGGCATCTTTAAAAGTGAGCT |
| | ATCTGGACTTCAGAGGCTCATAGCACCCTCCTGTGCTGCAGCCTTTCT |
| | CACCTGGAAGATATCTCCTCC |
| M-INB, (Mouse | GCAGCTGTCGACCTGAGTTATATGCACAAGATCCTGGAATATTTTCAT |
| Inteferon B): −807 to | TATCCTGGAGACACTGTGTTACTAATAACTCTGTATTTGTTCCTGCCA |
| +57: PCR = 864 bp | GTACTTACTCTCTCCTGTGGCCCCTCATTGTCCTTGCTCCTTTAGAAGT |
| (SEQ ID NO: 23) | GGCCCAAGTTGGTTTTTCTCACTTCTCAGAGCTCCCTCTTCTGTTGCTT |
| | TGGTGAAGTGCTGGGAAACTTGGCTGCTTCTCTCTCTGTTACCCACTC |
| | ACAACCCTTCCTTCCCTGGTCTCTTGTTTTGACACTGCTCAAACTGGA |
| | CCCCCACTTTAAGCAGTTTCTCCAGGATGTGGGGTTCTCTCCCAAAGG |
| | GAGCTATGGCCCATTGATGTGCCATTTCTCTTATAGTACACTATTGTA |
| | AGCCCCCTGGGAGCTCACAGAGACCCTCTCCCACCATCCCACTATTTC |
| | AAAGGGCAGCAGCTGTGCCATCCTTTCAGGATGAGCAGCTACTCTGC |
| | CTGGCTTTTCAGTGGACACTTTGGCTGTTTGAGAGTTCTTTTATCTTCA |
| | GGGCTGTCTCCTTTCTGTTCTTCTCTCCTGGATATTTCTCTTCCTTTGCT |
| | CCAGCAATTGGTGAAACTGTACAAGATTTTATAAATCCTTAGTTTGTA |
| | TATATTTTAACCCAGTACATAGCATATAAAATAGCCAGGAGCTTGAA |
| | TAAAATGAATATTAGAAGCTGTTAGAATAAGAGAAAATGACAGAGG |
| | AAAACTGAAAGGGAGAACTGAAAGTGGGAAATTCCTCTGAGGCAGA |
| | AAGGACCATCCCTTATAAATAGCACAGGCCATGAAGGAAGATCATTC |
| | TCACTGCAGCCTTTGACAGCCTTTGCCTCATCTTGCAGATATCAGCCG |
| | AC |
| H-CBOX1 (Human | GTCGACACTCTGTCTCAAAAATAATATCAATAGTAATAATGGTAATA |
| Carboxypeptidase | CAAACAAAAATACTGATAAATCTCAAAAACATTATCTTGAATGAAAG |
| B1) | AAGCCAGAAACAAAGGAATATATATTGTATAATCTCATTTACATTAA |
| SEQ ID NO: 24 | GTTTAATACAGGCAAAATTAATGTATAAAGCCAGAAATCAGAGTAAC |
| | ATTTGCCTGAGAGGAGTACAGAAGTAGACTGGAAAAGTGCTCAAGG |
| | GAACTTGTTAGGGTGATGAAAATGTGCTATATCTTGTTTGGGATGTTT |
| | ATTACATGGGTTTACACATTTGTCAAAACTCATCAAACTTTACACTAA |
| | AATCTGTAGAACTTATTGTATGTAATTCTCAATTCAAAACAGAAAAA |
| | AATACAAAATAGATTTTATTTAAATCTAATTTACTCTCAGACTATTCA |
| | AGTTCTGATCCAAATTAAATAAGTTGAATTTGAGAGTATTTACTCTAT |
| | TTCCACAGGTGTTGAGAGAGATACCCTCTCAGACTTTACACAGCTACC |
| | AAGGCAACAATGCCAGGAAAACTATTAATCAAGATATGATAGTTCCT |
| | GTTTGCAATCAGGAGAAGAAAGCAAAGAACACTCAGGATTATAAAA |
| | GCAGATGAGACCTACCCACTAGACCTGGTCAGATATC |
| H-REG1 (Human | GTCGACTCCATGATTGTAAGTTTCCTGAGGCCTCCCCATCCTCAGGAA |
| Regenerating Islet- | AGTCTGTGAGTCAATTAAACTATGAGTCAATTAAACCTCTTTCCTTTT |
| Derived 1B) | TAAGTTACCCAGTCTCTGGTATTATCTTTATAGCAGTATGAAAAGGAC |
| (SEQ ID NO: 25) | TAATACACCTTGCCTTCTGAAGTAGGTCTTGAGAGGTTCCTGAACCTG |
| | AAAGGGAATGGAGAAATGACTTAAAGCCACCAGCTAATTGGGCTTAA |
| | TCATTTGGACTCAGTTGACTCTTCCCCTACCCCTACCCATGCCTAAAC |
| | CAAAGAAAGGATCATCCCACATTTACCTAGCACAAAGAAATCTACTC |
| | TTCTGCTCTTTCTAGGACTGCTAAAGGCCATGGGAACTGGACACCTGG |
| | ATGCTGCAGAGGAAGGGCAAAGCTCAACATCAACTTGGACAGTTTGC |
| | CAACCTGTTTGTGGTAAGTTGATGTCATTTGTGACCACTCCTAATGTG |
| | TGCCAGGAATAAGCTATTCCTGATGCCAGAATCTCTTACTGTCAGTGC |
| | CCTCTGTAGGCCTTCTGATCCTTACTCCTTGCTCCACCCATTGTTTATA |
| | TCATGTAGTTCTCTCTCAGACCCTGATATAAAGCTCCTACTCTGTCTG |
| | ACCTGACAAGCCACCTCAAGTGGACAAGGCACTTACCAACAGGTAAA |
| | GGGGCATTACAGGAGAAGAGGATATCTGAAGCTCGAGGGCTCG |
| H-TDOX (Human | GCTACGTCGACCAGGCTGGTCTTGAACTACTGTTTTAAAATTCTATAT |
| Tryptophan 2, 3 | AGGCAATGTATGCTAATGTGGATGACTGCTAGCTACCCTGTCATTGGT |
| Dioxygenase) | ATGCCACAGATGAATAGTAAACTGATAATGTTCATTTAAATTCCAAT |
| (SEQ ID NO: 26) | ACAATGTGTTGAGAGCTTAACTGCTGCTGATTATTACATGATATTTAG |
| | GTCAGAAGAACTCTGATGATTGAATGGAAGTGGGCTTTGAGCATGTA |
| | CCCAGACTTTATTATACAGTTAATTTCCCTAGAGGTGTTAAATTTCAG |
| | TTTTTGGTCTAGAAGCTGTAAGAATATTCAAATTTGAGAGAATACATA |
| | TTTGTTTTTTACAAGCTCATCATCTTATGTATCCTAATACTATGCCAAA |
| | TGTTTTCTTGGGATTGAATAAATGTAATTTTTTTAACTTGGCCTCTGTT |
| | GATTCATTGGATGTTTGTTACTTTGAATATAAAAGCAGAACTTGGCAA |
| | GTCATACAACTCAATTTGGCAGATCACTCACTCTTAGCAAATGAACTG |
| | TGCCCAACATAAATCCACTCTAAAGTTTACAGAGGTTCAGTTCTGTAC |
| | CAATGGAAATGAGAAGTTAGCTAAGAGTACAAGAAGCATGCTGATTG |
| | GCTGATGCAGGGTAAGCAGGCTACATAAAAGGCAGCTGTAGAACATC |
| | TGGGAAGGTCAATGATATCATCTG |

Linker

| | |
|---|---|
| 1x Linker | GGTGGAGGAGGTAGT |
| (SEQ ID NO: 27) | |
| 2x Linker | GGTGGAGGAGGTAGTGGGGGTGGAGGTTCA |
| (SEQ ID NO: 28) | |

Matrix Attachment Regions (MARs)

| | |
|---|---|
| βGlo MAR | TTAATTAAAATTATCTCTAAGGCATGTGAACTGGCTGTCTTGGTTTTCAT |
| SEQ ID NO: 29 | CTGTACTTCATCTGCTACCTCTGTGACCTGAAACATATTTATAATTCCAT |
| | TAAGCTGTGCATATGATAGATTTATCATATGTATTTTCCTTAAAGGATTTT |
| | TGTAAGAACTAATTGAATTGATACCTGTAAAGTCTTTATCACACTACCC |
| | AATAAATAATAAATCTCTTTGTTCAGCTCTCTGTTTCTATAAATATGTAC |
| | CAGTTTTATTGTTTTTAGTGGTAGTGATTTTATTCTCTTTCTATATATATAC |
| | ACACACATGTGTGCATTCATAAATATATACAATTTTTATGAATAAAAAAT |
| | TATTAGCAATCAATATTGAAAACCACTGATTTTTGTTTATGTGAGCAAA |
| | CAGCAGATTAAAAGG |
| 21421 S/MAR | GAGCCCCACTGTGTTCATCTTACAGATGGAAATACTGACATTCAGAGG |
| (SEQ ID NO: 30) | AGTTAGTTAACTTGCCTAGGTGATTCAGCTAATAAGTGCAAGAAAGAT |
| | TTCAATCCAAGGTGATTTGATTCTGAAGCCTGTGCTAATCACATTACAC |
| | CAAGCTACAACTTCATTTATAAATAATAAGTCAGCTTTCAAGGGCCTTT |
| | CAGGTGTCCTGCACTTCTACAAGCTGTGCCATTTAGTGAACACAAAAT |
| | GAGCCTTCTGATGAAGTAGTCTTTTCATTATTTCAGATATTAGAACACT |
| | AAAATTCTTAGCTGCCAGCTGATTGAAGGCTGGGACAAAATTCAAACA |
| | TGCATCTACAACAATATATATCTCAATGTTAGTCTCCAAATTCTATTGAC |
| | TTCAACTCAAGAGAATATAAAGAGCTAGTCTTTATACACTCTTTAAGGT |
| | ATGATATCATCTGGAAAGTAACAAAATTGATGCAAATTTGAATGAACTT |
| | TATCATGGTGTATTTACACAATGTGTTTCTTCTCCCTGCAATGTATTTCT |
| | TTCTCTAATTCCTTCCATTTGATCTTTCATACACAATCTGGTTCTGATGT |
| | ATGTTTTTTGGATGCACTTTTCAACTCCAAAAGACAGAGCTAGTTACTT |
| | TCTTCCTGGTGCTCCAAGCACTGTATTTGTATCTGTATTCAAGCCCTTT |
| | GCAATATTGTACTGGATCATTATTTCACCTCTAGGATGGCTTCCCCAGG |
| | CAACTTGTGTTCACCCAGAGACTACATTTTGTATCTTGTTGACCTTTGA |
| | ACTTCCACCAGTGTCTAAAAATAATATGTATGCAAAATTACTTGCTATG |
| | AGAATGTATAATTAAACAATATAAAAAGGAGAAGCAAGGAGAGAAAC |
| | ACAGGTGTGTATTTGTGTTTGTGTGCTTAAAAGGCAGTGTGGAAAAGG |
| | AAGAAATGCCATTTATAGTGAGGAGACAAAGTTATATTACCTCTTATCT |
| | GGCTTTTAAGGAGATTTTGCTGAGCTAAAAATCCTATATTCATAGAAAA |
| | GCCTTACCTGAGTTGCCAATACC |
| IFNβ S/MAR | AGTCAATATGTTCACCCCAAAAAAGCTGTTTGTTAACTTGTCAACCTCA |
| (SEQ ID NO: 31) | TTCTAAAATGTATATAGAAGCCCAAAAGACAATAACAAAAATATTCTTG |
| | TAGAACAAAATGGGAAAGAATGTTCCACTAAATATCAAGATTTAGAGC |
| | AAAGCATGAGATGTGTGGGGATAGACAGTGAGGCTGATAAAATAGAGT |
| | AGAGCTCAGAAACAGACCCATTGATATATGTAAGTGACCTATGAAAAA |
| | AATATGGCATTTTACAATGGGAAAATGATGGTCTTTTTCTTTTTTAGAA |
| | AAACAGGGAAATATATTTATATGTAAAAAATAAAAGGGAACCCATATGT |
| | CATACCATACACACAAAAAAATTCCAGTGAATTATAAGTCTAAATGGA |
| | GAAGGCAAAACTTTAAATCTTTTAGAAAATAATATAGAAGCATGCCATC |
| | AAGACTTCAGTGTAGAGAAAAATTTCTTATGACTCAAAGTCCTAACCA |
| | CAAAGAAAAGATTGTTAATTAGATTGCATGAATATTAAGACTTATTTTT |
| | AAAATTAAAAAACCATTAAGAAAAGTCAGGCCATAGAATGACAGAAA |
| | ATATTTGCAACACCCCAGTAAAGAGAATTGTAATATGCAGATTATAAAA |
| | AGAAGTCTTACAAATCAGTAAAAAATAAAACTAGACAAAAATTTGAA |
| | CAGATGAAAGAGAAACTCTAAATAATCATTACACATGAGAAACTCAAT |
| | CTCAGAAATCAGAGAACTATCATTGCATATACACTAAATTAGAGAAATA |
| | TTAAAAGGCTAAGTAACATCTGTGGC |

Super-Enhancer

| | |
|---|---|
| HR3 | AAAACAAATGACATCATTCCTGATTATAATAATTTTAATTGTGCTTTACA |
| (SEQ ID NO: 32) | AGTAGAATTCTACTTGTAAAGAGAGTTTAATTTGAAAAACAAATTAGT |
| | CATTATTAAACATGTTAACAATTGTGTATAAAAATGACATCAGTTTAATG |
| | ATGACATCATCTCTTGATTATGTTTTACAAGTAGAATTCTACTTGTAAAG |
| | CTGGTTCAGTTTTGAAAAACAAATGACATCATCTCTTGATTATGTTTTA |
| | CAAGTAGAATTCTACTTGTAAAAGTGAGTTTAGTTTTAAAAAACAAAT |
| | GACATCATTCAGTTTTGAAAAACAAATGACATCATCTCTTGATTGTGTT |
| | TTACAAGTAGAATTCTACTTGTAAAGTGAGTTCAGTTTTGAAAAACAA |
| | ATGACCCTCTCATACAATTGTTGAACAATTTTAATAAATAATCTTTACAA |
| | GATT |
| SE1 | TCCTGCAGTTAAGGAAGCCCTGGGCCTGCTGTTCTCATCCACTGAGCT |
| (SEQ ID NO: 33) | CTTCTGCCCTGGAGAAGCCCACTTGTTAGCTTATTACAGTTCATGGACA |
| | CCAGGTCCACAATGTAAGCTAACAGAAACTATGTCTGCTTTCCCATCC |
| | GTCTCAGTGACATGGAGGGGGGGGGGGGGCCACTGGCGATCCTCAAG |
| | ACCCAAACCATCCTGAGGATTTGGTTCCATTTGGCTCTCCCTAGCAGCT |
| | GCACTTGCAGGAAGAACAGCTCAGCTCACACTGCCTCTCCTCTGGCCT |
| | CACTGTGCTCAAAGGACAGGCCTCAGGCCACAGGAGGCTTTGTGCAC |
| | TGCGCCGAGTGGAGCAGTGGAGCGTCTGGGAAAAGTTGCAAACCGA |
| | GCTGGAAGACTTGCTTGGATGAGAAACTGGCGTAGGACCGAGTCTGT |
| | TTGCCCCATCAAGCTGGCCAGGCCAGATCCACCTTCCTGCCCCACAGA |
| | TGGGGAAGGCTCCTACCCATTGTTCTCCAGGATGCTTTGACAGAGAGG |
| | AAAGGGAATGTAGGCCCTGGAGACCGGAAGGTTCAGCTTCCGGGCAC |
| | CAAGCTCTGCCAGACTCACGCCTGCCCTCCAGACAGGAAGGCAGGTC |
| | CTTGGTCTCATTCTGTCCTGCCCAGCTGCTGGACCACCCCCAATCAATG |
| | CTTTCCCCTTTCTGGGCCCTTAAGGAGACGGCATACAGGAGGCTTTGT |
| | CTGTGTGCTAGCCTGGGAAGGGAATTCCAGAGGAATTGAGGAGAATA |
| | ATGCCCAAGATTTGGGGCTCCCTGGTCCCAGCTATACCATTCAGTGTAC |
| | CATTACACTCCAAGTACTGCATATGGCCACAGAAATACCTTGTATGCTC |
| | TGTGGCTGGCCTCACACATTCACATTATCAGACAAATGCCATGTGCAC |
| | AATCTTAGTTCCATATACACTC |
| SE2 | AGTGATCCTCAACTTCTGGGTCTTGATCCCTTTGGGGGCTGAACAACC |
| (SEQ ID NO: 34) | AATTAACAGGGGTAACCTACGAGCATCAGAAAACATAGATCTTTACATT |
| | ATGATTCATAGCAGTAGCAAAATGACAGCTATGAAGTAGCAACGAAAT |
| | ACTTCTATGGTTGGGGGTCACCACAACATGAAATATGGTACTAAGGGA |
| | TCGTGGCTTTAGGAAGGTTGAGAAAGCCGATCCCAGCCAGTCCTTGTC |
| | AGGCTCTGCGCTGAGCCTTTTCAGCCTGGACTTTCCAGCTTCAGGGTC |
| | CTTGGTCCTCTGGCACCACTTCCTCAACAACATTGAAAGGATGTCTGA |
| | GAGAAAGCAAGACAGCTGGGAAAGTGCTCTCAGAGGAGCTAGTCCC |
| | CCTCCGCTCTAATGGCCATTGTTCTCACCACCAGGAATGCAGTTCTTGC |
| | TCTGCTCCTTCCTGGCCTGGGACAGACTGCCCAACACCCCTCTTCCTC |
| | CTCAGGCTCCTGAGGCTGCAGCGTTAACTGCCTTAGCCTCTGGCCTCT |
| | TCGGTGGTCAAGATCATGCCCCATCCCCCCACCCTGCTCGCTCTCGAG |
| | AGTGTGCCTTCGCTCTCTCGCTCTCTCGTGTTCTCTCTCTCTCTCTCTCT |
| | CTCTCTCTCTCTCTCTCACACACACACACACACACACACACACACACA |
| | AAACCACCCCATGGGGGATCAAAACCTACACAGGACAATCTTCCTAA |
| | AACAGAACCTCTTAGTTTGAGCCCATGGTTTCAAAGCTGCTTGGGGGA |
| | CATCAGGCCACCTCTCTGACAATACCTAGTATGAAAGC |
| SE3 | ATCTGCATTTCCAACTCCTCTCTCCACCCTAATGATCCTGAGGCTTGAG |
| (SEQ ID NO: 35) | GACCATGCTCATTAAAAAAATATATTTATTTTTATTGTATGCATATTGGTA |
| | TTTTGCCTCTCTATACATCTGTGCAACCATGGGCATGCAGTGCAGGAGG |
| | GAGCCAGAAGATGGCATTAGATCCTCTGGAACCAGAGCTACAGACAC |
| | CTATTTGTGGCCAGAAAGGTACTAGTAGCAGAAACAAGGTCCTCTGGA |
| | AGAGCAACTGATGCTCTTAGGTACTGAAGCATCATCCTGCCCCAGAGA |
| | CCACTCGCATATGAAGCACACATATTCAGTCTGCCTTACTTGTGTTAAT |
| | GATTGCCAGTGTCCCTCTGACCTCCTAGCCCTGAAAAGTGTGGCCTGA |
| | AGGTCATTTCAGAGACGGGGAGAGCTGCTCAGAGAAGCCAATCGGCG |
| | AGTCTAGGACACACAGACAGGATCTAGTCCCAGAGTTCGCTAGCCTAG |
| | GTGAGCGTCCCCTGGCCCCTTATACCACTTCCTTCTCCAGCTTGCATCT |
| | AATCTGCTCTGGCAGACCATCGTGTTTCCTGTCTTCCTGGCAGCCTCCA |
| | GCACGCTCAGTGCTACTCCCTGCGCATGCGCCCTCCTCCCAGTACCTT |
| | CTCTGACTCCAGTGGGCTTGGAGTGCGAGGAGGAAGGGTGAGGAAG |
| | GGGTGAAATCAGGTATTGGATCCACAGGGGGTCTGAAGAGCACTAGC |
| | CTGGCCTTTTGGGACTGAACTTCTGCTATGAAGACCTCCACTGCCATC |
| | CCTGGAGTCCGGGGCACATCCAAGGCTTGCCTGTCCATCGTTTACTGT |
| | TTACAGATGACAACAATGACTGTGTTCGGGGCAGAAATATCCACCAGG |
| | GCTAGAGTACAAAAGGAGTTTGCATTGATGGCCGGACAGGCCCTGTCC |
| | CTGGCAGCCTGCCAGCGCTGAGTATGAGACCCAGCGGGAAGTGCTAC |
| | CCTGGCAGACGTGTCCACTGAGTACACAGACCACCAAGGCAGGCAGC |
| | TCTCGGGGAAGCTGTCTATGCTGGGCCAGCCCACCTTGAGGGCAGGG |
| | AACAGAACAGATTGTGGCAGAGAGGAAAATGTGGAGCTTCTGTTTGT |
| | TCACAGACACACGCACTCGCCCACGCACGCACGCACGCACGCACGCA |
| | CGCACGAATGCACGCACGCAGTAGTTGAATGCTATGGATTCCGCTCAG |
| | AGCTGAGAACAGCCCCAGCGACAGTTCCCTGGCCTCTCTCCTTACTCT |
| | GATGTCCTCATCTGTCTTCACATGGTCTCAGGACGCTAATACTCCATCC |
| | TAATGTACACTCCTTTCCCTGGGCCTCCGTTCCAGTTCAGTTCTCAGAG |
| | GACCTGGAGGGA |
| SE4 | TCATTATCCGCATCACCACTACCATCACCATTTCTACTTTACAGGCAGT |
| (SEQ ID NO: 36) | GGTGCTTAGGTTTGAGGAAGTCACCCACAAGAAGATTGTAGAAGTGG |
| | GACCCCGTTCTTCAGCAGCAGCCTCAGCCCCACTCCTGAGCAGGTCCT |
| | GTGACAACTCATCTCCCTGCTGGTCCTGGGGAGTGGGGAAGGGACCC |
| | CATCACTGACACTGTCTTGTTCCCAACCGGTAAGAGTCAGGGGTTCTG |
| | CCCACCATCTGGAGACCTTCGCATAGGTCCAGATTCGAGCTCTTTCTTT |
| | TAAGCACAGGGTGTTCTCTTCCACGTTATCTGTCTGCGTGGGCCACAA |
| | ACCCGGTTTATAAACAAGGCAAAAAATGGCAAGAGGAAAAAGTGAAG |
| | TCAGCCTGTGCCATTGGAGCAAGTCAAACAATACCAAGCTCGTAGCTG |
| | GCTAAGTGGCTGTCTCATTGTCCAGATATTTCCTCAGGCCATTAAACAA |
| | GCGAGGAATGATTGCCAGGTTTGATTTCAGGAAGGAAGCCCCTCCCAT |
| | TTCCCTGCCGAGCAATTCAGTGTTCATTTTCTTCTGACCCACGCTGGCT |
| | GGTTATTAGGGAAGCGCTGCATGCTTCTTTCATAAACAAAAGTTATTTG |
| | CTTCAAGATTCAAAATTCACTTCTCAACGACTGGGAAGCCCTGAGTTC |
| | CTGCCAGATGGTTTTATTATTTTGGAGGTAGGGCTAGCCCAAGTACTGG |
| | GAGCACCCTGCGACCAGAGTAGACTTTGGGGGCTGAGGGAAAGCCAT |
| | TCTGCCACTGGCCTGGAGTTCAGAGCCTTTGTCCAGAGAGCCCCTTTG |
| | GGTGGAAGACAAAGTAACTAGGTTGGTTCGGCAAAGTTCCGACGGCT |
| | GGCCCGCAGCCCTGCCTTGGGTGGCCACAGATACACACGGCCATGGG |
| | AACAGCCAGTGAACTTTTTTTTTTTTTTTTCTTAATCAGGGTCCTACTC |
| | TGCCTGTGGAGCAAGCTTGTCATTATTCTCACCTCTTGGGAGACAGTC |
| | ACTTTTTTTTTTTTTCCTTTTGAGACAAGGTTTCTCTGTGTAACAG |
| SE5 | GGGGTGGGGGCGGGGGTAGGGGAGGAGCAGGGTGCAGCTGCTCACT |
| (SEQ ID NO: 37) | GCCCTTCCCCCTCTCATGTCTCCTTGGCAGCAGCTGCCTGCTAAAGAC |
| | TAGCCCAGGCCAGGAGCTGTTTGTCTCTGCTTGTCAGAGGCGTTTCCT |
| | GATGGGTCTCTATTTTGGTTTCTGTTTTTCTCTGTGCTAACAGAGAAAG |
| | AGACGGCTCCAACGTCATGGGGTTTTCCCAGCAATTAGCCTCGGAGCA |
| | AACAGACAGCAAATCGGGATTTAATGAGTTCCTTCCCGTCCTCCTCCA |
| | TCCGCAGTGACCACTTGGTGAGCTGCCAGCCAGGGTTAGATCTGGAC |
| | CCGGTCCCCATAGCCTCCTCCAATTGTGTAGGATGAGAGACCTTGAGA |
| | ACAGGGGACCAGGCTGGCAGGCACTAGCTCATATTTAGTCATCA |

Longterm Repeat (LTR) within intron

| | |
|---|---|
| PPRV | AGGGGCCAAGTCCATACGACAGGCCCCAGCCCGCCAGACGGAGGGCC |
| (SEQ ID NO: 38) | GCAGAAAGGAAGGAGACACGACCGCCCCAGAAGGAGGA |
| MV | AGGGCCAAGGAACATACACACCCAACAGAACCCAGACCCCGGCCCAC |
| (SEQ ID NO: 39) | GGCGCCGCGCCCCCAACCCCCGACAACCAGAGGGAGCC |
| SNV | GCCGGGTACATCTCTTGCTCGGGGTCGCCGTCCTACACATTGTTGTTGT |
| (SEQ ID NO: 40) | GACGTGCGGCCCAGATTCGAATCTGTAATAAAACCTTTTTTTTTCTGAA |
| | TCCTCAGATTGGCAGTGAGAGGAGATTTTGTTCGTGGTGTTGGCTCGC |
| | CTACTGGGTGGGCGCAGGGATCCGGACTGAATCCGTAGTACTTCGGTA |
| | CAACATTTGGGGG |
| RU5 | GGTCGCCGTCCTACACATTGTTGTTGTGACGTGCGGCCCAGATTCGAA |
| (SEQ ID NO: 41) | TCTGTATAAAACTTTTTTTTTTCTGAATCCTCAGATTGGCAGTGAGAGG |
| | AGATTTTGTTCGTGGTGTTGGCTCGCCTACTGGGTGGGCGCAGGGATC |
| | CGGACTGAATCCGTAGTACT |

Microtubule-associated Sequence (NLS-MTAS)

| | |
|---|---|
| NLS-MTAS | GRYLTQETNKVETYKEQPLKTPGKKKKGKPGKRKEQEKKKRRTR |
| (SEQ ID NO: 42) | |
| NLS-MTAScoding | GGCAGATACCTGACACAAGAGACAAACAAGGTGGAAACCTACAAAG |
| sequence | AGCAGCCCCTGAAAACCCCAGGCAAGAAGAAGAAGGGCAAGCCTGG |
| (SEQ ID NO: 43) | AAAGAGGAAAGAACAAGAGAAAAAGAAGAGAAGGACCAGA |
| NLS-MTAS- | GRYLTQETNKVETYKEQPLKTPGKKKKGKPGKRKEQEKKKRRTRDRDD |
| Hydrophillic Linker- | QAAWFSQY |
| Modified Neck | |
| Domain Peptide | |
| (SEQ ID NO: 44) | |
| NLS-MTAS- | GGCAGATACCTGACACAAGAGACAAACAAGGTGGAAACCTACAAAG |
| Hydrophillic Linker- | AGCAGCCCCTGAAAACCCCAGGCAAGAAGAAGAAGGGCAAGCCTGG |
| Modified Neck | AAAGAGGAAAGAACAAGAGAAAAAGAAGAGAAGGACCAGAGACAG |
| Domain Peptide | GGATGACCAGGCTGCTTGGTTTTCTCAGTAC |
| coding sequence | |
| (SEQ ID NO: 45) | |

In certain embodiments, one or more (e.g., at least 3, or at least 8 antibodies) are expressed with the systems and methods herein. In some embodiments, this includes the therapeutic monoclonal antibodies (mAbs), Fabs, F(ab)2s, and scFv's that are shown in Table 4 below, as well as the anti-SARS-CoV2 antibodies 5 and antigen bindings provided at Table 5 and Table 7, which is herein incorporated by reference.

TABLE 4

| A | B | | | | |
|---|---|---|---|---|---|
| Antibody | Trade | C | D | E | F |
| Name | name | Type | Source | Target | Use |
| 3F8 | | mab | mouse | GD2 | neuroblastoma |
| | | | | ganglioside | |
| 8H9 | | mab | mouse | B7-H3 | neuroblastoma, |
| | | | | | sarcoma, metastatic |
| | | | | | brain cancers |
| Abagovomab | | mab | mouse | CA-125 | ovarian cancer |
| | | | | (imitation) | |
| Abciximab | ReoPro | Fab | chimeric | CD41 (integrin | platelet aggregation |
| | | | | alpha-IIb) | inhibitor |
| Abituzumab | | mab | humanized | CD51 | cancer |
| Abrilumab | | mab | human | integrin α4β7 | inflammatory |
| | | | | | bowel disease, |
| | | | | | ulcerative colitis, |
| | | | | | Crohn's disease |
| Actoxumab | | mab | human | Clostridium | Clostridium |
| | | | | difficile | difficile colitis |
| Adalimumab | Humira | mab | human | TNF-α | Rheumatoid |
| | | | | | arthritis, Crohn's |
| | | | | | Disease, Plaque |
| | | | | | Psoriasis, Psoriatic |
| | | | | | Arthritis, |
| | | | | | Ankylosing |
| | | | | | Spondylitis, |
| | | | | | Juvenile Idiopathic |
| | | | | | Arthritis, |
| | | | | | Hemolytic disease |
| | | | | | of the newborn |
| Adecatumumab | | mab | human | EpCAM | prostate and breast |
| | | | | | cancer |
| Aducanumab | | mab | human | beta-amyloid | Alzheimer's disease |
| Afasevikumab | | mab | human | IL17A and | — |
| | | | | IL17F | |
| Afelimomab | | F(ab')₂ | mouse | TNF-α | sepsis |
| Afutuzumab | | mab | humanized | CD20 | lymphoma |
| Alacizumab pegol | | F(ab')₂ | humanized | VEGFR2 | cancer |
| ALD518 | | — | humanized | IL-6 | rheumatoid arthritis |
| Alemtuzumab | Lemtrada, | mab | humanized | CD52 | Multiple sclerosis |
| | Campath | | | | |
| Alirocumab | | mab | human | PCSK9 | hypercholesterolemia |
| Altumomab | Hybri- | mab | mouse | CEA | colorectal cancer |
| pentetate | ceaker | | | | (diagnosis) |
| Amatuximab | | mab | chimeric | mesothelin | cancer |
| Anatumomab | | Fab | mouse | TAG-72 | non-small cell lung |
| mafenatox | | | | | carcinoma |
| Anetumab | | mab | human | MSLN | cancer |
| ravtansine | | | | | |
| Anifrolumab | | mab | human | interferon α/β | systemic lupus |
| | | | | receptor | erythematosus |
| Anrukinzumab (= | | mab | humanized | IL-13 | asthma |
| IMA-638) | | | | | |
| Apolizumab | | mab | humanized | HLA-DR— | hematological |
| | | | | | cancers |
| Arcitumomab | CEA-Scan | Fab' | mouse | CEA | gastrointestinal |
| | | | | | cancers (diagnosis) |
| Ascrinvacumab | | mab | human | activin | cancer |
| | | | | receptor-like | |
| | | | | kinase 1 | |
| Aselizumab | | mab | humanized | L-selectin | severely injured |
| | | | | (CD62L) | patients |
| Atezolizumab | | mab | humanized | CD274 | cancer |
| Atinumab | | mab | human | RTN4 | — |
| Atlizumab (= | Actemra, | mab | humanized | IL-6 receptor | rheumatoid arthritis |
| tocilizumab) | RoActemra | | | | |
| Atorolimumab | | mab | human | Rhesus factor | hemolytic disease |
| | | | | | of the |
| | | | | | newborn[citation |
| | | | | | needed] |
| Avelumab | | mab | human | CD274 | — |
| Bapineuzumab | | mab | humanized | beta amyloid | Alzheimer's disease |
| Basiliximab | Simulect | mab | chimeric | CD25 (α chain | prevention of organ |
| | | | | of IL-2 | transplant |
| | | | | receptor) | rejections |
| Bavituximab | | mab | chimeric | phosphatidylser | cancer, viral |
| | | | | ine | infections |
| Bectumomab | LymphoScan | Fab' | mouse | CD22 | non-Hodgkin's |
| | | | | | lymphoma |
| | | | | | (detection) |
| Begelomab | | mab | mouse | DPP4 | — |
| Belimumab | Benlysta, | mab | human | BAFF | non-Hodgkin |
| | LymphoSta | | | | lymphoma etc. |
| | t-B | | | | |
| Benralizumab | | mab | humanized | CD 125 | asthma |
| Bertilimumab | | mab | human | CCL11 | severe allergic |
| | | | | (eotaxin-1) | disorders |
| Besilesomab | Scintimun | mab | mouse | CEA-related | inflammatory |
| | | | | antigen | lesions and |
| | | | | | metastases |
| | | | | | (detection) |
| Bevacizumab | Avastin | mab | humanized | VEGF-A | metastatic cancer, |
| | | | | | retinopathy of |
| | | | | | prematurity |
| Bezlotoxumab | | mab | human | Clostridium | Clostridium |
| | | | | difficile | difficile colitis |
| Biciromab | FibriScint | Fab' | mouse | fibrin II, beta | thromboembolism |
| | | | | chain | (diagnosis) |
| Bimagrumab | | mab | human | ACVR2B | myostatin inhibitor |
| Bimekizumab | | mab | humanized | IL 17A and IL | — |
| | | | | 17F | |
| Bivatuzumab | | mab | humanized | CD44 v6 | squamous cell |
| mertansine | | | | | carcinoma |
| Bleselumab | | mab | human | CD40 | — |
| Blinatumomab | | BiTE | mouse | CD19 | pre-B ALL |
| | | | | | (CD 19+) |
| Blontuvetmab | Biontress | mab | veterinary | CD20 | — |
| Blosozumab | | mab | humanized | SOST | osteoporosis |
| Bococizumab | | mab | humanized | neural | dyslipidemia |
| | | | | apoptosis- | |
| | | | | regulated | |
| | | | | proteinase 1 | |
| Brazikumab | | mab | human | IL23 | Crohn's disease |
| Brentuximab | | mab | chimeric | CD30 | hematologic |
| vedotin | | | | (TNFRSF8) | cancers |
| Briakinumab | | mab | human | IL-12, IL-23 | psoriasis, |
| | | | | | rheumatoid |
| | | | | | arthritis, |
| | | | | | inflammatory |
| | | | | | bowel diseases, |
| | | | | | multiple sclerosis |
| Brodalumab | | mab | human | IL-17 | inflammatory |
| | | | | | diseases |
| Brolucizumab | | mab | humanized | VEGFA | wet age-related |
| | | | | | macular |
| | | | | | degeneration |
| Brontictuzumab | | mab | humanized | Notch 1 | cancer |
| Burosumab | | mab | human | FGF23 | X-linked |
| | | | | | hypophosphatemia |
| Cabiralizumab | | mab | humanized | CSF1R | — |
| Canakinumab | Ilaris | mab | human | IL-1— | rheumatoid arthritis |
| Cantuzumab | | mab | humanized | mucin CanAg | colorectal cancer |
| mertansine | | | | | etc. |
| Cantuzumab | | mab | humanized | MUC1 | cancers |
| ravtansine | | | | | |
| Caplacizumab | | mab | humanized | VWF | thrombotic |
| | | | | | thrombocytopenic |
| | | | | | purpura, |
| | | | | | thrombosis |
| Capromab | Prostascint | mab | mouse | prostatic | prostate cancer |
| pendetide | | | | carcinoma cells | (detection) |
| Carlumab | | mab | human | MCP-1 | oncology/immune |
| | | | | | indications |
| Carotuximab | | mab | chimeric | endoglin | — |
| Catumaxomab | Removab | 3funct | rat/mouse | EpCAM, CD3 | ovarian cancer, |
| | | | hybrid | | malignant ascites, |
| | | | | | gastric cancer |
| cBR96- | | mab | humanized | Lewis-Y | cancer |
| doxorubicin | | | | antigen | |
| immunoconjugate | | | | | |
| Cedelizumab | | mab | humanized | CD4 | prevention of organ |
| | | | | | transplant |
| | | | | | rejections, |
| | | | | | treatment of |
| | | | | | autoimmune |
| | | | | | diseases |
| Cergutuzumab | | mab | humanized | IL2 | — |
| amunaleukin | | | | | |
| Certolizumab | Cimzia | Fab' | humanized | TNF-α | Crohn's disease |
| pegol | | | | | Rheumatoid |
| | | | | | arthritis axial |
| | | | | | spondyloarthritis |
| | | | | | psoriasis arthritis |
| Cetuximab | Erbitux | mab | chimeric | EGFR | metastatic |
| | | | | | colorectal cancer |
| | | | | | and head and neck |
| | | | | | cancer |
| Ch.14.18 | | mab | chimeric | GD2 | neuroblastoma |
| | | | | ganglioside | |
| Citatuzumab | | Fab | humanized | EpCAM | ovarian cancer and |
| bogatox | | | | | other solid tumors |
| Cixutumumab | | mab | human | IGF-1 receptor | solid tumors |
| | | | | (CD221) | |
| Clazakizumab | | mab | humanized | Oryctolagus | rheumatoid arthritis |
| | | | | cuniculus | |
| Clenoliximab | | mab | chimeric | CD4 | rheumatoid arthritis |
| Clivatuzumab | hPAM4- | mab | humanized | MUC1 | pancreatic cancer |
| tetraxetan | Cide | | | | |
| Codrituzumab | | mab | humanized | glypican 3 | cancer |
| Coltuximab | | mab | chimeric | CD19 | cancer |
| ravtansine | | | | | |
| Conatumumab | | mab | human | TRAIL-R2 | cancer |
| Concizumab | | mab | humanized | TFPI | bleeding |
| CR6261 | | mab | human | Influenza A | infectious |
| | | | | hemagglutinin | disease/influenza A |
| Crenezumab | | mab | humanized | 1-40-β-amyloid | Alzheimer's disease |
| Crotedumab | | mab | human | GCGR | diabetes |
| Dacetuzumab | | mab | humanized | CD40 | hematologic |
| | | | | | cancers |
| Daclizumab | Zenapax | mab | humanized | CD25 (α chain | prevention of organ |
| | | | | of IL-2 | transplant |
| | | | | receptor) | rejections |
| Dalotuzumab | | mab | humanized | IGF-1 receptor | cancer etc. |
| | | | | (CD221) | |
| Dapirolizumab | | mab | humanized | CD 154 | — |
| pegol | | | | (CD40L) | |
| Daratumumab | | mab | human | CD38 (cyclic | cancer |
| | | | | ADP ribose | |
| | | | | hydrolase) | |
| Dectrekumab | | mab | human | IL-13 | — |
| Demcizumab | | mab | humanized | DLL4 | cancer |
| Denintuzumab | | mab | humanized | CD19 | cancer |
| mafodotin | | | | | |
| Denosumab | Prolia | mab | human | RANKL | osteoporosis, bone |
| | | | | | metastases etc. |
| Depatuxizumab | | mab | chimeric/ | EGFR | cancer |
| mafodotin | | | humanized | | |
| Derlotuximab | | mab | chimeric | histone | recurrent |
| biotin | | | | complex | glioblastoma |
| | | | | | multiforme |
| Detumomab | | mab | mouse | B-lymphoma | lymphoma |
| | | | | cell | |
| Dinutuximab | | mab | chimeric | GD2 | neuroblastoma |
| | | | | ganglioside | |
| Diridavumab | | mab | human | hemagglutinin | influenza A |
| Domagrozumab | | mab | humanized | GDF-8 | Duchenne muscular |
| | | | | | dystrophy |
| Dorlimomab | | F(ab′)₂ | mouse | — | — |
| aritox | | | | | |
| Drozitumab | | mab | human | DR5 | cancer etc. |
| Duligotumab | | mab | human | ERBB3 | testicular cancer |
| | | | | (HER3) | |
| Dupilumab | | mab | human | IL4 | atopic diseases |
| Durvalumab | | mab | human | CD274 | cancer |
| Dusigitumab | | mab | human | ILGF2 | cancer |
| Ecromeximab | | mab | chimeric | GD3 | malignant |
| | | | | ganglioside | melanoma |
| Eculizumab | Soliris | mab | humanized | C5 | paroxysmal |
| | | | | | nocturnal |
| | | | | | hemoglobinuria, |
| | | | | | atypical HUS |
| Edobacomab | | mab | mouse | endotoxin | sepsis caused by |
| | | | | | Gram-negative |
| | | | | | bacteria |
| Edrecolomab | Panorex | mab | mouse | EpCAM | colorectal |
| | | | | | carcinoma |
| Efalizumab | Raptiva | mab | humanized | LFA-1 | psoriasis (blocks T- |
| | | | | (CD11a) | cell migration) |
| Efungumab | Mycograb | scFv | human | Hsp90 | invasive Candida |
| | | | | | infection |
| Eldelumab | | mab | human | interferon | Crohn's disease, |
| | | | | gamma- | ulcerative colitis |
| | | | | induced protein | |
| Elgemtumab | | mab | human | ERBB3 | cancer |
| | | | | (HER3) | |
| Elotuzumab | | mab | humanized | SLAMF7 | multiple myeloma |
| Elsilimomab | | mab | mouse | IL-6 | — |
| Emactuzumab | | mab | humanized | CSF1R | cancer |
| Emibetuzumab | | mab | humanized | HHGFR | cancer |
| Emicizumab | | mab | humanized | activated F9, | haemophilia A |
| | | | | F10 | |
| Enavatuzumab | | mab | humanized | TWEAK | cancer etc. |
| | | | | receptor | |
| Enfortumab | | mab | human | AGS-22M6 | cancer expressing |
| vedotin | | | | | Nectin-4 |
| Enlimomab pegol | | mab | mouse | ICAM-1 | — |
| | | | | (CD54) | |
| Enoblituzumab | | mab | humanized | CD276 | cancer |
| Enokizumab | | mab | humanized | IL9 | asthma |
| Enoticumab | | mab | human | DLL4 | — |
| Ensituximab | | mab | chimeric | 5AC | cancer |
| Epitumomab | | mab | mouse | episialin | — |
| cituxetan | | | | | |
| Epratuzumab | | mab | humanized | CD22 | cancer, SLE |
| Erenumab | | mab | human | CGRP | migraine |
| Erlizumab | | F(ab′)₂ | humanized | ITGB2 (CD18) | heart attack, stroke, |
| | | | | | traumatic shock |
| Ertumaxomab | Rexomun | 3funct | rat/mouse | HER2/neu, | breast cancer etc. |
| | | | hybrid | CD3 | |
| Etaracizumab | Abegrin | mab | humanized | integrin αvβ3 | melanoma, prostate |
| | | | | | cancer, ovarian |
| | | | | | cancer etc. |
| Etrolizumab | | mab | humanized | integrin α7 β7 | inflammatory |
| | | | | | bowel disease |
| Evinacumab | | mab | human | angiopoietin 3 | dyslipidemia |
| Evolocumab | | mab | human | PCSK9 | hyper- |
| | | | | | cholesterolemia |
| Exbivirumab | | mab | human | hepatitis B | hepatitis B |
| | | | | surface antigen | |
| Fanolesomab | NeutroSpec | mab | mouse | CD15 | appendicitis |
| | | | | | (diagnosis) |
| Faralimomab | | mab | mouse | interferon | — |
| | | | | receptor | |
| Farletuzumab | | mab | humanized | folate receptor | ovarian cancer |
| | | | | 1 | |
| Fasinumab | | mab | human | HNGF | acute sciatic pain |
| FBTA05 | Lymphomun | 3funct | rat/mouse | CD20 | chronic |
| | | | hybrid | | lymphocytic |
| | | | | | leukaemia |
| Felvizumab | | mab | humanized | respiratory | respiratory |
| | | | | syncytial virus | syncytial virus |
| | | | | | infection |
| Fezakinumab | | mab | human | IL-22 | rheumatoid |
| | | | | | arthritis, psoriasis |
| Fibatuzumab | | mab | humanized | ephrin receptor | — |
| | | | | A3 | |
| Ficlatuzumab | | mab | humanized | HGF | cancer etc. |
| Figitumumab | | mab | human | IGF-1 receptor | adrenocortical |
| | | | | (CD221) | carcinoma, non- |
| | | | | | small cell lung |
| | | | | | carcinoma etc. |
| Firivumab | | mab | human | influenza A | — |
| | | | | virus | |
| | | | | hemagglutinin | |
| Flanvotumab | | mab | human | TYRP1(glycop | melanoma |
| | | | | rotein 75) | |
| Fletikumab | | mab | human | IL 20 | rheumatoid arthritis |
| Fontolizumab | HuZAF | mab | humanized | IFN-γ | Crohn's disease etc. |
| Foralumab | | mab | human | CD3 epsilon | — |
| Foravirumab | | mab | human | rabies virus | rabies |
| | | | | glycoprotein | (prophylaxis) |
| Fresolimumab | | mab | human | TGF-β | idiopathic |
| | | | | | pulmonary fibrosis, |
| | | | | | focal segmental |
| | | | | | glomerulosclerosis, |
| | | | | | cancer |
| Fulranumab | | mab | human | NGF | pain |
| Futuximab | | mab | chimeric | EGFR | cancer |
| Galcanezumab | | mab | humanized | calcitonin | migraine |
| Galiximab | | mab | chimeric | CD80 | B-cell lymphoma |
| Ganitumab | | mab | human | IGF-1 receptor | cancer |
| | | | | (CD221) | |
| Gantenerumab | | mab | human | beta amyloid | Alzheimer's disease |
| Gavilimomab | | mab | mouse | CD147 | graft versus host |
| | | | | (basigin) | disease |
| Gemtuzumab | Mylotarg | mab | humanized | CD33 | acute myelogenous |
| ozogamicin | | | | | leukemia |
| Gevokizumab | | mab | humanized | IL-1β | diabetes etc. |
| Girentuximab | Rencarex | mab | chimeric | carbonic | clear cell renal cell |
| | | | | anhydrase 9 | carcinoma[84] |
| | | | | (CA-IX) | |
| Glembatumumab | | mab | human | GPNMB | melanoma, breast |
| vedotin | | | | | cancer |
| Golimumab | Simponi | mab | human | TNF-α | rheumatoid |
| | | | | | arthritis, psoriatic |
| | | | | | arthritis, ankylosing |
| | | | | | spondylitis |
| Gomiliximab | | mab | chimeric | CD23 (IgE | allergic asthma |
| | | | | receptor) | |
| Guselkumab | | mab | human | IL23 | psoriasis |
| Ibalizumab | | mab | humanized | CD4 | HIV infection |
| Ibritumomab | Zevalin | mab | mouse | CD20 | non-Hodgkin's |
| tiuxetan | | | | | lymphoma |
| Icrucumab | | mab | human | VEGFR-1 | cancer etc. |
| Idarucizumab | | mab | humanized | dabigatran | reversal of |
| | | | | | anticoagulant |
| | | | | | effects of |
| | | | | | dabigatran |
| Igovomab | Indimacis- | F(ab′)₂ | mouse | CA-125 | ovarian cancer |
| | 125 | | | | (diagnosis) |
| IMAB362 | | mab | human | CLDN18.2 | gastrointestinal |
| | | | | | adenocarcinomas |
| | | | | | and pancreatic |
| | | | | | tumor |
| Imalumab | | mab | human | MIF | cancer |
| Imciromab | Myoscint | mab | mouse | cardiac myosin | cardiac imaging |
| Imgatuzumab | | mab | humanized | EGFR | cancer |
| Inclacumab | | mab | human | selectin P | cardiovascular |
| | | | | | disease |
| Indatuximab | | mab | chimeric | SDC1 | cancer |
| ravtansine | | | | | |
| Indusatumab | | mab | human | GUCY2C | cancer |
| vedotin | | | | | |
| Inebilizumab | | mab | humanized | CD19 | cancer, systemic |
| | | | | | sclerosis, multiple |
| | | | | | sclerosis |
| Infliximab | Remicade | mab | chimeric | TNF-α | rheumatoid |
| | | | | | arthritis, ankylosing |
| | | | | | spondylitis, |
| | | | | | psoriatic arthritis, |
| | | | | | psoriasis, Crohn's |
| | | | | | disease, ulcerative |
| | | | | | colitis |
| Inolimomab | | mab | mouse | CD25 (α chain | graft versus host |
| | | | | of IL-2 | disease |
| | | | | receptor) | |
| Inotuzumab | | mab | humanized | CD22 | ALL |
| ozogamicin | | | | | |
| Intetumumab | | mab | human | CD51 | solid tumors |
| | | | | | (prostate cancer, |
| | | | | | melanoma) |
| Ipilimumab | Yervoy | mab | human | CD152 | melanoma |
| Iratumumab | | mab | human | CD30 | Hodgkin's |
| | | | | (TNFRSF8) | lymphoma |
| Isatuximab | | mab | chimeric | CD38 | cancer |
| Itolizumab | | mab | humanized | CD6 | — |
| Ixekizumab | | mab | humanized | IL 17A | autoimmune |
| | | | | | diseases |
| Keliximab | | mab | chimeric | CD4 | chronic asthma |
| Labetuzumab | CEA-Cide | mab | humanized | CEA | colorectal cancer |
| Lampalizumab | | mab | humanized | CFD | geographic atrophy |
| | | | | | secondary to age- |
| | | | | | related macular |
| | | | | | degeneration |
| Lanadelumab | | mab | human | kallikrein | angioedema |
| Landogrozumab | | mab | humanized | GDF-8 | muscle wasting |
| | | | | | disorders |
| Laprituximab | | mab | chimeric | EGFR | — |
| emtansine | | | | | |
| Lebrikizumab | | mab | humanized | IL-13 | asthma |
| Lemalesomab | | mab | mouse | NCA-90 | diagnostic agent |
| | | | | (granulocyte | |
| | | | | antigen) | |
| Lendalizumab | | mab | humanized | C5 | — |
| Lenzilumab | | mab | human | CSF2 | — |
| Lerdelimumab | | mab | human | TGF beta 2 | reduction of |
| | | | | | scarring after |
| | | | | | glaucoma surgery |
| Lexatumumab | | mab | human | TRAIL-R2 | cancer |
| Libivirumab | | mab | human | hepatitis B | hepatitis B |
| | | | | surface antigen | |
| Lifastuzumab | | mab | humanized | phosphate- | cancer |
| vedotin | | | | sodium co- | |
| | | | | transporter | |
| Ligelizumab | | mab | humanized | IGHE | severe asthma and |
| | | | | | chronic |
| | | | | | spontaneous |
| | | | | | urticaria |
| Lilotomab | | mab | mouse | CD37 | cancer |
| satetraxetan | | | | | |
| Lintuzumab | | mab | humanized | CD33 | cancer |
| Lirilumab | | mab | human | KIR2D | solid and |
| | | | | | hematological |
| | | | | | cancers |
| Lodelcizumab | | mab | humanized | PCSK9 | hyper- |
| | | | | | cholesterolemia |
| Lokivetmab | | mab | veterinary | Canis lupus | — |
| | | | | familiaris IL31 | |
| Lorvotuzumab | | mab | humanized | CD56 | cancer |
| mertansine | | | | | |
| Lucatumumab | | mab | human | CD40 | multiple myeloma, |
| | | | | | non-Hodgkin's |
| | | | | | lymphoma, |
| | | | | | Hodgkin's |
| | | | | | lymphoma |
| Lulizumab pegol | | mab | humanized | CD28 | autoimmune |
| | | | | | diseases |
| Lumiliximab | | mab | chimeric | CD23 (IgE | chronic |
| | | | | receptor) | lymphocytic |
| | | | | | leukemia |
| Lumretuzumab | | mab | humanized | ERBB3 | cancer |
| | | | | (HER3) | |
| MABp1 | Xilonix | mab | human | IL1A | colorectal cancer |
| Mapatumumab | | mab | human | TRAIL-R1 | cancer |
| Margetuximab | | mab | humanized | ch4D5 | cancer |
| Maslimomab | | | mouse | T-cell receptor | — |
| Matuzumab | | mab | humanized | EGFR | colorectal, lung and |
| | | | | | stomach cancer |
| Mavrilimumab | | mab | human | GMCSF | rheumatoid arthritis |
| | | | | receptor α- | |
| | | | | chain | |
| Mepolizumab | Bosatria | mab | humanized | IL-5 | asthma and white |
| | | | | | blood cell diseases |
| Metelimumab | | mab | human | TGF beta 1 | systemic |
| | | | | | scleroderma |
| Milatuzumab | | mab | humanized | CD74 | multiple myeloma |
| | | | | | and other |
| | | | | | hematological |
| | | | | | malignancies |
| Minretumomab | | mab | mouse | TAG-72 | tumor detection |
| | | | | | (and therapy ) |
| Mirvetuximab | | mab | chimeric | folate receptor | cancer |
| soravtansine | | | | alpha | |
| Mitumomab | | mab | mouse | GD3 | small cell lung |
| | | | | ganglioside | carcinoma |
| Mogamulizumab | | mab | humanized | CCR4 | cancer |
| Monalizumab | | mab | humanized | KLRC1 | — |
| Morolimumab | | mab | human | Rhesus factor | — |
| Motavizumab | Numax | mab | humanized | respiratory | respiratory |
| | | | | syncytial virus | syncytial virus |
| | | | | | (prevention) |
| Moxetumomab | | mab | mouse | CD22 | cancer |
| pasudotox | | | | | |
| Muromonab-CD3 | Orthoclone | mab | mouse | CD3 | prevention of organ |
| | OKT3 | | | | transplant |
| | | | | | rejections |
| Nacolomab | | Fab | mouse | C242 antigen | colorectal cancer |
| tafenatox | | | | | |
| Namilumab | | mab | human | CSF2 | — |
| Naptumomab | | Fab | mouse | 5T4 | non-small cell lung |
| estafenatox | | | | | carcinoma, renal |
| | | | | | cell carcinoma |
| Naratuximab | | mab | chimeric | CD37 | — |
| emtansine | | | | | |
| Narnatumab | | mab | human | RON | cancer |
| Natalizumab | Tysabri | mab | humanized | integrin α4 | multiple sclerosis, |
| | | | | | Crohn's disease |
| Navicixizumab | | mab | chimeric/ | DLL4 | — |
| | | | humanized | | |
| Navivumab | | mab | human | influenza A | — |
| | | | | virus | |
| | | | | hemagglutinin | |
| | | | | HA | |
| Nebacumab | | mab | human | endotoxin | sepsis |
| Necitumumab | | mab | human | EGFR | non-small cell lung |
| | | | | | carcinoma |
| Nemolizumab | | mab | humanized | IL31RA | eczema[106] |
| Nerelimomab | | mab | mouse | TNF-α | — |
| Nesvacumab | | mab | human | angiopoietin 2 | cancer |
| Nimotuzumab | Theracim, | mab | humanized | EGFR | squamous cell |
| | Theraloc | | | | carcinoma, head |
| | | | | | and neck cancer, |
| | | | | | nasopharyngeal |
| | | | | | cancer, glioma |
| Nivolumab | Opdivo | mab | human | PD-1 | cancer |
| Nofetumomab | Verluma | Fab | mouse | | cancer (diagnosis) |
| merpentan | | | | | |
| Obiltoxaximab | | mab | chimeric | Bacillus | Bacillus anthracis |
| | | | | anthracis | spores |
| | | | | anthrax | |
| Obinutuzumab | Gazyva | mab | humanized | CD20 | Chronic lymphatic |
| | | | | | leukemia |
| Ocaratuzumab | | mab | humanized | CD20 | cancer |
| Ocrelizumab | | mab | humanized | CD20 | rheumatoid |
| | | | | | arthritis, lupus |
| | | | | | erythematosus etc. |
| Odulimomab | | mab | mouse | LFA-1 | prevention of organ |
| | | | | (CD11a) | transplant |
| | | | | | rejections, |
| | | | | | immunological |
| | | | | | diseases |
| Ofatumumab | Arzerra | mab | human | CD20 | chronic |
| | | | | | lymphocytic |
| | | | | | leukemia etc. |
| Olaratumab | | mab | human | PDGF-R α | cancer |
| Olokizumab | | mab | humanized | IL6 | — |
| Omalizumab | Xolair | mab | humanized | IgE Fc region | allergic asthma |
| Onartuzumab | | mab | humanized | human scatter | cancer |
| | | | | factor receptor | |
| | | | | kinase | |
| Ontuxizumab | | mab | chimeric/ | TEM1 | cancer |
| | | | humanized | | |
| Opicinumab | | mab | human | LINGO-1 | multiple sclerosis |
| Oportuzumab | | scFv | humanized | EpCAM | cancer |
| monatox | | | | | |
| Oregovomab | OvaRex | mab | mouse | CA-125 | ovarian cancer |
| Orticumab | | mab | human | oxLDL | — |
| Otelixizumab | | mab | chimeric/ | CD3 | diabetes mellitus |
| | | | humanized | | type 1 |
| Otlertuzumab | | mab | humanized | CD37 | cancer |
| Oxelumab | | mab | human | OX-40 | asthma |
| Ozanezumab | | mab | humanized | NOGO-A | ALS and multiple |
| | | | | | sclerosis |
| Ozoralizumab | | mab | humanized | TNF-α | inflammation |
| Pagibaximab | | mab | chimeric | lipoteichoic | sepsis |
| | | | | acid | (Staphylococcus) |
| Palivizumab | Synagis, | mab | humanized | F protein of | respiratory |
| | Abbosynagis | | | respiratory | syncytial virus |
| | | | | syncytial virus | (prevention) |
| Pamrevlumab | | mab | human | CTGF | — |
| Panitumumab | Vectibix | mab | human | EGFR | colorectal cancer |
| Pankomab | | mab | humanized | tumor specific | ovarian cancer |
| | | | | glycosylation | |
| | | | | ofMUC1 | |
| Panobacumab | | mab | human | Pseudomonas | Pseudomonas |
| | | | | aeruginosa | aeruginosa |
| | | | | | infection |
| Parsatuzumab | | mab | human | EGFL7 | cancer |
| Pascolizumab | | mab | humanized | IL-4 | asthma |
| Pasotuxizumab | | mab | chimeric/ | folate | cancer |
| | | | humanized | hydrolase | |
| Pateclizumab | | mab | humanized | LTA | TNF |
| Patritumab | | mab | human | ERBB3 | cancer |
| | | | | (HER3) | |
| Pembrolizumab | | mab | humanized | PDCD1 | melanoma and |
| | | | | | other cancers |
| Pemtumomab | Theragyn | | mouse | MUC1 | cancer |
| Perakizumab | | mab | humanized | IL 17A | arthritis |
| Pertuzumab | Omnitarg | mab | humanized | HER2/neu | cancer |
| Pexelizumab | | scFv | humanized | C5 | reduction of side |
| | | | | | effects of cardiac |
| | | | | | surgery |
| Pidilizumab | | mab | humanized | PD-1 | cancer and |
| | | | | | infectious diseases |
| Pinatuzumab | | mab | humanized | CD22 | cancer |
| vedotin | | | | | |
| Pintumomab | | mab | mouse | adenocarcinom | adenocarcinoma |
| | | | | a antigen | (imaging) |
| Placulumab | | mab | human | human TNF | pain and |
| | | | | | inflammatory |
| | | | | | diseases |
| Plozalizumab | | mab | humanized | CCR2 | diabetic |
| | | | | | nephropathy and |
| | | | | | arteriovenous graft |
| | | | | | patency |
| Pogalizumab | | mab | humanized | TNFR | — |
| | | | | superfamily | |
| | | | | member 4 | |
| Polatuzumab | | mab | humanized | CD79B | cancer |
| vedotin | | | | | |
| Ponezumab | | mab | humanized | human beta- | Alzheimer's disease |
| | | | | amyloid | |
| Prezalizumab | | mab | humanized | ICOSL | — |
| Priliximab | | mab | chimeric | CD4 | Crohn's disease, |
| | | | | | multiple sclerosis |
| Pritoxaximab | | mab | chimeric | E. coli shiga | — |
| | | | | toxin type-1 | |
| Pritumumab | | mab | human | vimentin | brain cancer |
| PRO 140 | | | humanized | CCR5 | HIV infection |
| Quilizumab | | mab | humanized | IGHE | asthma |
| Racotumomab | | mab | mouse | N- | cancer |
| | | | | glycolylneuraminic | |
| | | | | acid | |
| Radretumab | | mab | human | fibronectin | cancer |
| | | | | extra domain-B | |
| Rafivirumab | | mab | human | rabies virus | rabies |
| | | | | glycoprotein | (prophylaxis) |
| Ralpancizumab | | mab | humanized | neural | dyslipidemia |
| | | | | apoptosis- | |
| | | | | regulated | |
| | | | | proteinase 1 | |
| Ramucirumab | Cyramza | mab | human | VEGFR2 | solid tumors |
| Ranibizumab | Lucentis | Fab | humanized | VEGF-A | macular |
| | | | | | degeneration (wet |
| | | | | | form) |
| Raxibacumab | | mab | human | anthrax toxin, | anthrax |
| | | | | protective | (prophylaxis and |
| | | | | antigen | treatment) |
| Refanezumab | | mab | humanized | myelin- | recovery of motor |
| | | | | associated | function after |
| | | | | glycoprotein | stroke |
| Regavirumab | | mab | human | cytomegalovirus | cytomegalovirus |
| | | | | glycoprotein | infection |
| | | | | B | |
| Reslizumab | | mab | humanized | IL-5 | inflammations of |
| | | | | | the airways, skin |
| | | | | | and gastrointestinal |
| | | | | | tract |
| Rilotumumab | | mab | human | HGF | solid tumors |
| Rinucumab | | mab | human | platelet-derived | neovascular age- |
| | | | | growth factor | related macular |
| | | | | receptor beta | degeneration |
| Risankizumab | | mab | humanized | IL23A | — |
| Rituximab | MabThera, | mab | chimeric | CD20 | lymphomas, |
| | Rituxan | | | | leukemias, some |
| | | | | | autoimmune |
| | | | | | disorders |
| Rivabazumab | | mab | humanized | Pseudomonas | — |
| pegol | | | | aeruginosa type | |
| | | | | III secretion | |
| | | | | system | |
| Robatumumab | | mab | human | IGF-1 receptor | cancer |
| | | | | (CD221) | |
| Roledumab | | mab | human | RHD | — |
| Romosozumab | | mab | humanized | sclerostin | osteoporosis |
| Rontalizumab | | mab | humanized | IFN-α | systemic lupus |
| | | | | | erythematosus |
| Rovalpituzumab | | mab | humanized | DLL3 | — |
| tesirine | | | | | |
| Rovelizumab | LenkArrest | mab | humanized | CD11, CD18 | haemorrhagic |
| | | | | | shock etc. |
| Ruplizumab | Antova | mab | humanized | CD154 | rheumatic diseases |
| | | | | (CD40L) | |
| Sacituzumab | | mab | humanized | tumor- | cancer |
| govitecan | | | | associated | |
| | | | | calcium signal | |
| | | | | transducer 2 | |
| Samalizumab | | mab | humanized | CD200 | cancer |
| Sapelizumab | | mab | humanized | IL6R | — |
| Sarilumab | | mab | human | IL6 | rheumatoid |
| | | | | | arthritis, ankylosing |
| | | | | | spondylitis |
| Satumomab | | mab | mouse | TAG-72 | cancer (diagnosis) |
| pendetide | | | | | |
| Secukinumab | | mab | human | IL 17A | uveitis, rheumatoid |
| | | | | | arthritis psoriasis |
| Seribantumab | | mab | human | ERBB3 | cancer |
| | | | | (HER3) | |
| Setoxaximab | | mab | chimeric | E. coli shiga | — |
| | | | | toxin type-2 | |
| Sevirumab | | | human | cytomegalovirus | cytomegalovirus |
| | | | | | infection |
| SGN-CD19A | | mab | humanized | CD19 | acute |
| | | | | | lymphoblastic |
| | | | | | leukemia and B- |
| | | | | | cell non-Hodgkin |
| | | | | | lymphoma |
| SGN-CD33A | | mab | humanized | CD33 | Acute myeloid |
| | | | | | leukemia |
| Sibrotuzumab | | mab | humanized | FAP | cancer |
| Sifalimumab | | mab | humanized | IFN-α | SLE, |
| | | | | | dermatomyositis, |
| | | | | | polymyositis |
| Siltuximab | | mab | chimeric | IL-6 | cancer |
| Simtuzumab | | mab | humanized | LOXL2 | fibrosis |
| Siplizumab | | mab | humanized | CD2 | psoriasis, graft- |
| | | | | | versus-host disease |
| | | | | | (prevention) |
| Sirukumab | | mab | human | IL-6 | rheumatoid arthritis |
| Sofituzumab | | mab | humanized | CA-125 | ovarian cancer |
| vedotin | | | | | |
| Solanezumab | | mab | humanized | beta amyloid | Alzheimer's disease |
| Solitomab | | BiTE | mouse | EpCAM | — |
| Sonepcizumab | | — | humanized | sphingosine-1- | choroidal and |
| | | | | phosphate | retinal |
| | | | | | neovascularization |
| Sontuzumab | | mab | humanized | episialin | — |
| Stamulumab | | mab | human | myostatin | muscular dystrophy |
| Sulesomab | LeukoScan | Fab' | mouse | NCA-90 | osteomyelitis |
| | | | | (granulocyte | (imaging) |
| | | | | antigen) | |
| Suvizumab | | mab | humanized | HIV-1 | viral infections |
| Tabalumab | | mab | human | BAFF | B-cell cancers |
| Tacatuzumab | AFP-Cide | mab | humanized | alpha- | cancer |
| tetraxetan | | | | fetoprotein | |
| Tadocizumab | | Fab | humanized | integrin αIIbβ3 | percutaneous |
| | | | | | coronary |
| | | | | | intervention |
| Talizumab | | mab | humanized | IgE | allergic reaction |
| Tamtuvetmab | Tactress | mab | veterinary | CD52 | — |
| Tanezumab | | mab | humanized | NGF | pain |
| Taplitumomab | | mab | mouse | CD19 | cancer[citation |
| paptox | | | | | needed] |
| Tarextumab | | mab | human | Notch receptor | cancer |
| Tefibazumab | Aurexis | mab | humanized | clumping factor | Staphylococcus |
| | | | | A | aureus infection |
| Telimomab aritox | | Fab | mouse | — | — |
| Tenatumomab | | mab | mouse | tenascin C | cancer |
| Teneliximab | | mab | chimeric | CD40 | autoimmune |
| | | | | | diseases and |
| | | | | | prevention of organ |
| | | | | | transplant rejection |
| Teplizumab | | mab | humanized | CD3 | diabetes mellitus |
| | | | | | type 1 |
| Teprotumumab | | mab | human | IGF-1 receptor | hematologic tumors |
| | | | | (CD221) | |
| Tesidolumab | | mab | human | C5 | — |
| Tetulomab | | mab | humanized | CD37 | cancer[141] |
| Tezepelumab | | mab | human | TSLP | asthma, atopic |
| | | | | | dermatitis |
| TGN1412 | | — | humanized | CD28 | chronic |
| | | | | | lymphocytic |
| | | | | | leukemia, |
| | | | | | rheumatoid arthritis |
| Ticilimumab (= | | mab | human | CTLA-4 | cancer |
| tremelimumab) | | | | | |
| Tigatuzumab | | mab | humanized | TRAIL-R2 | cancer |
| Tildrakizumab | | mab | humanized | IL23 | immunologically |
| | | | | | mediated |
| | | | | | inflammatory |
| | | | | | disorders |
| Timolumab | | mab | human | AOC3 | — |
| Tisotumab vedotin | | mab | human | coagulation | — |
| | | | | factor III | |
| TNX-650 | | — | humanized | IL-13 | Hodgkin's |
| | | | | | lymphoma |
| Tocilizumab (= | Actemra, | mab | humanized | IL-6 receptor | rheumatoid arthritis |
| atlizumab) | RoActemra | | | | |
| Toralizumab | | mab | humanized | CD154 | rheumatoid |
| | | | | (CD40L) | arthritis, lupus |
| | | | | | nephritis etc. |
| Tosatoxumab | | mab | human | Staphylococcus | — |
| | | | | aureus | |
| Tositumomab | Bexxar | — | mouse | CD20 | follicular |
| | | | | | lymphoma |
| Tovetumab | | mab | human | CD 140a | cancer |
| Tralokinumab | | mab | human | IL-13 | asthma etc. |
| Trastuzumab | Herceptin | mab | humanized | HER2/neu | breast cancer |
| Trastuzumab | Kadcyla | mab | humanized | HER2/neu | breast cancer |
| emtansine | | | | | |
| TRBS07 | Ektomab | 3funct | — | GD2 | melanoma |
| | | | | ganglioside | |
| Tregalizumab | | mab | humanized | CD4 | — |
| Tremelimumab | | mab | human | CTLA-4 | cancer |
| Trevogrumab | | mab | human | growth | muscle atrophy due |
| | | | | differentiation | to orthopedic |
| | | | | factor 8 | disuse and |
| | | | | | sarcopenia |
| Tucotuzumab | | mab | humanized | EpCAM | cancer |
| celmoleukin | | | | | |
| Tuvirumab | | — | human | hepatitis B | chronic hepatitis B |
| | | | | virus | |
| Ublituximab | | mab | chimeric | MS4A1 | cancer |
| Ulocuplumab | | mab | human | CXCR4 | hematologic |
| | | | | (CD184) | malignancies |
| Urelumab | | mab | human | 4-1BB | cancer etc. |
| | | | | (CD137) | |
| Urtoxazumab | | mab | humanized | Escherichia | diarrhoea caused by |
| | | | | coli | E. coli |
| Ustekinumab | Stelara | mab | human | IL-12, IL-23 | multiple sclerosis, |
| | | | | | psoriasis, psoriatic |
| | | | | | arthritis |
| Utomilumab | | mab | human | 4-1BB | cancer |
| | | | | (CD137) | |
| Vadastuximab | | mab | chimeric | CD33 | — |
| talirine | | | | | |
| Vandortuzumab | | mab | humanized | STEAP1 | cancer |
| vedotin | | | | | |
| Vantictumab | | mab | human | Frizzled | cancer |
| | | | | receptor | |
| Vanucizumab | | mab | humanized | angiopoietin 2 | cancer |
| Vapaliximab | | mab | chimeric | AOC3 (VAP-1) | — |
| Varlilumab | | mab | human | CD27 | solid tumors and |
| | | | | | hematologic |
| | | | | | malignancies |
| Vatelizumab | | mab | humanized | ITGA2 | — |
| | | | | (CD49b) | |
| Vedolizumab | Entyvio | mab | humanized | integrin α4β7 | Crohn's disease, |
| | | | | | ulcerative colitis |
| Veltuzumab | | mab | humanized | CD20 | non-Hodgkin's |
| | | | | | lymphoma |
| Vepalimomab | | mab | mouse | AOC3 (VAP-1) | inflammation |
| Vesencumab | | mab | human | NRP1 | solid malignancies |
| Visilizumab | Nuvion | mab | humanized | CD3 | Crohn's disease, |
| | | | | | ulcerative colitis |
| Vobarilizumab | | mab | humanized | IL6R | inflammatory |
| | | | | | autoimmune |
| | | | | | diseases |
| Volociximab | | mab | chimeric | integrin α5β1 | solid tumors |
| Vorsetuzumab | | mab | humanized | CD70 | cancer |
| mafodotin | | | | | |
| Votumumab | HumaSPE | mab | human | tumor antigen | colorectal tumors |
| | CT | | | CTAA16.88 | |
| Xentuzumab | | mab | | IGF1, IGF2 | — |
| Zalutumumab | HuMax- | mab | human | EGFR | squamous cell |
| | EGFr | | | | carcinoma of the |
| | | | | | head and neck |
| Zanolimumab | HuMax- | mab | human | CD4 | rheumatoid |
| | CD4 | | | | arthritis, psoriasis, |
| | | | | | T-cell lymphoma |
| Zatuximab | | mab | chimeric | HER1 | cancer |
| Ziralimumab | | mab | human | CD147 | — |
| | | | | (basigin) | |
| Zolimomab aritox | | mab | mouse | CD5 | systemic lupus |
| | | | | | erythematosus, |
| | | | | | graft-versus-host |
| | | | | | disease |

TABLE 5

| Sponsors | Drug code | Trial IDs |
|---|---|---|
| Celltrion | CT-P63 | NCT05017168 |
| Exevir Bio BV | XVR011 | NCT04884295 |
| Jemincare Group | JMB2002 | ChiCTR2100042150 |
| Luye Pharma Group Ltd | LY-CovMab | NA |
| AbbVie | ABBV-47D11 | NCT04644120 |
| HiFiBiO Therapeutics | HFB30132A | NCT04590430 |
| Ology Bioservices | ADM03820 | NCT04592549 |
| Beigene | DXP604 | NCT04669262 |
| Zydus Cadila | ZRC-3308 | NA |
| Hengenix Biotech Inc | HLX70 | NCT04561076 |
| CORAT Therapeutics | COR-101 | NCT04674566 |
| Vir Biotechnol./ | VIR-7832 | NCT04746183 |
| AbCellera/Eli Lilly and | LY-CoV1404, | NCT04634409 |
| Company | LY3853113 | |
| Sorrento Therapeutics, | COVI-AMG (STI- | NCT04734860 |
| Inc. | 2020) | |
| Beigene | DXP593 | NCT04532294; |
| | | NCT04551898 |
| Junshi Biosciences/ | JS016, LY3832479, | NCT04441918; |
| Eli Lilly | LY-CoV016 | NCT04441931; |
| and Company | | NCT04427501 |
| Mabwell (Shanghai) | MW33 | NCT04533048; |
| Bioscience Co., Ltd. | | NCT04627584 |
| Toscana Life Sciences | MAD0004J08 | NCT04932850; |
| Sviluppo s.r.l. | | NCT04952805 |
| Bristol-Myers Squibb, | C144-LS and C-135-LS | NCT04700163; |
| Rockefeller University | | Activ-2 study |
| Sinocelltech Ltd. | SCTA01 | NCT04483375; |
| | | NCT04644185 |
| Adagio Therapeutics | ADG20 | NCT04805671 |
| | | NCT04859517 |
| Brii Biosciences | BRII-196 | NCT04479631; |
| | | Activ-3 study |
| Brii Biosciences | BRII-198 | NCT04479644; |
| | | Activ-3 study |
| Tychan Pte. Ltd. | TY027 | NCT04429529; |
| | | NCT04649515 |
| AstraZeneca | AZD7442 (AZD8895 + | NCT04507256; |
| | AZD1061) | NCT04625725; |
| | | NCT04625972 |
| Celltrion | CT-P59 | NCT04525079; |
| | | NCT04593641; |
| | | NCT04602000 |
| Vir Biotechnol./ | VIR-7831/ | NCT04545060; |
| Glaxo SmithKline | GSK4182136 | Activ-3 study |
| AbCellera/Eli Lilly and | LY-CoV555 | NCT04411628 (Phase |
| Company | (LY3819253); | 1); NCT04427501 |
| | | (Phase 2); |
| | combination of LY- | NCT04497987 |
| | CoV555 with LY- | (Phase 3); |
| | CoV016 (LY3832479) | NCT04501978 (Activ-3 study); |
| | | NCT04518410 (Phase 2/3) |
| Regeneron | REGN-COV2 | NCT04425629 (Phase |
| | (REGN10933 + | 1/2); NCT04426695 |
| | REGN10987) | (Phase |
| | | 1/2); NCT04452318 (Phase 3) |

In certain embodiments, an agent, such as an anti-inflammatory agent or bioactive lipid, is used to increase the expression level and/or duration of any the therapeutic protein (or biologically active nucleic acid molecules) expressed from the vectors in the methods herein. In certain embodiments, anti-inflammatory agents (AILs) and bioactive lipids in Table 6 below can be used in the compositions and pre-treatment solutions herein.

TABLE 6

| | | |
|---|---|---|
| Docosahexaenoic Acid | (DHA) | 3/10/15% |
| Eicosapenaenoic Acid | (EPA) | 10/15% |
| Alpha Linolenic Acid | (ALA) | 3/10/15% |
| Maresin 1 | (MAR1) | 3% |
| Lipoxin A4 | (LA4) | 2% |
| 15-deoxy-12,14-Prostaglandin J2 | (15d) | 3% |
| Arachidonic Acid | (AA) | 10/15% |
| Eicosatetraynoic Acid | (ETA) | 10% |
| Docosapentaenoic Acid | (DPA) | 10/15% |
| Stearidonic Acid | (SA) | 10% |
| Retinoic Acid | (RA) | 10% |
| Trans Retinal | (TA) | 10% |
| 2-Arachidonoyl Glycerol | (AG) | 10% |
| Diallyl Disulfide | (DADS) | 10% |
| 3,3-Diindolylmethane | (DIM) | 10% |
| Prostaglandin E2 | (PE2) | 10% |
| Oleic Acid | (OA) | 5/10/15/30/50% |
| Alpha Tocopherol | (AT) | 2.5% |
| Sphingosine-1-Phosphate | (S-1-P) | 10% |
| Palmitoyl Sphingomyelin | (SPH) | 10% |

TABLE 7
TABLE 7

| | | | | | Not | | | | |
|---|---|---|---|---|---|---|---|---|---|
| | Ab | | | Neutral- | Neutral- | | | SEQ | |
| | or | | Doesn't | ising | ising | Protein + | | ID | |
| Name | Nb | Binds to | Bind to | Vs | Vs | Epitope | Origin | NO.: | VH or VHH |
| 0304-2F8 | Ab | SARS-CoV2 | | | SARS-CoV2 | S; Unk | B-cells; SARS-CoV2 | 63 | EVQLVQSGAEVSQPGESLKISCKGSGYSF |
| | | (weak) | | | | | Human Patient | | TGYWISWVRQMPGKGLEWMGIIYPGD |
| | | | | | | | | | SDTKYTPSFQGQVTISTDKSINTAYLQWS |
| | | | | | | | | | SLKASDTAMYYCARRGDGLYYYGMDV |
| | | | | | | | | | WGQGTTVTVSS |
| 0304-3H3 | Ab | SARS-CoV2 | | SARS-CoV2 | | S; S2 | B-cells; SARS-CoV2 | 64 | EVQLVESGPGLVKPSETLSLTCTASGGSI |
| | | | | | | | Human Patient | | STYYWSWIRQPPGKGLEWIGYIYYSGST |
| | | | | | | | | | NYNPSLKSRVTISVDTSKNQFSLKLSSVT |
| | | | | | | | | | AADTAVYYCARDRIAPVGKFFGWYFDLW |
| | | | | | | | | | GRGTLVTVSS |
| 0304- | Ab | SARS-CoV2 | | | SARS-CoV2 | S; S2 | B-cells; SARS-CoV2 | 65 | EVQLVESGGGLVQPGGSLRLSCAASGFT |
| 4A10 | | | | | | | Human Patient | | FSTYAMHWVRQAPGKGLEYVSGISSNG |
| | | | | | | | | | GSTYYANSVKGRFTISRDNSKNTLYIQM |
| | | | | | | | | | GSLRAEDMAVYYCARSSSRGFDYWGQ |
| | | | | | | | | | GTLVTVSS |
| 0304-4A2 | Ab | SARS-CoV2 | | | SARS-CoV2 | S; S1 (non | B-cells; SARS-CoV2 | 66 | EVQLVESGPGLVKPSETLSLTCAVSGDST |
| | | | | | | RBD) | Human Patient | | SSSSSYWDWIRQPPGKGLEWIGNIYYTG |
| | | | | | | | | | TTYYNPSLKSRVTISVDTSKDQFSLKLSS |
| | | | | | | | | | VTAADTAVYYCARELFTAVAGKGGIDYW |
| | | | | | | | | | GQGTLVTVSS |
| 0317-A1 | Ab | SARS-CoV2 | | | SARS-CoV2 | S; S2 | B-cells; SARS-CoV2 | 67 | QVQLVQSGGGVVQPGRSLRLSCAAPGF |
| | | | | | | | Human Patient | | TFSSYGMHWVRQAPGKGLEWVAVISY |
| | | | | | | | | | DGSNKYYADSVKGRFTISRDNSKNTLYL |
| | | | | | | | | | QMNSLRAEDTAVYYCAKDFKGGSSSWY |
| | | | | | | | | | TPEIEYGMDVWGQGTTVTVSS |
| 0317-A2 | Ab | SARS-CoV2 | | | SARS-CoV2 | S; S2 | B-cells; SARS-CoV2 | 68 | QVQLVQSGSELKKPGASVKVSCKASGYT |
| | | | | | | | Human Patient | | FTSYAMNWVRQAPGQGLEWMGWINT |
| | | | | | | | | | NTGNPTYAQGFTGRFVFSLDTSVSTAYL |
| | | | | | | | | | QISSLKAEDTAVYYCARLIRHEAHTYCSG |
| | | | | | | | | | GSCYSPDYYYGMDVWGQGTMVTVSS |
| 0317-A3 | Ab | SARS-CoV2 | | | SARS-CoV2 | S; Link | B-cells; SARS-CoV2 | 69 | EVQLLQSGGGLVQPGGSLRLSCVASGFT |
| | | (weak) | | | | | Human Patient | | FSSYEMNWVRQAPGKGLEWVSYISTSG |
| | | | | | | | | | STINYADSVKGRFSISRDNAKKSLYLQM |
| | | | | | | | | | NSLRAEDTAVYYCASNPPLGEPYFDIWG |
| | | | | | | | | | QGTMVTVSS |
| 0317-A7 | Ab | SARS-CoV2 | | | SARS-CoV2 | S; S1 (non | B-cells; SARS-CoV2 | 70 | EVQLLESGGGVVQPGRSLRLSCAASGFT |
| | | | | | | RBD) | Human Patient | | FSNYAMHWVRQAPGKGLEWVAVISYD |
| | | | | | | | | | GSNKYYADSVKGRFTISRDNSKNTLYLQ |
| | | | | | | | | | MNSLRAEDTAVYYCARWGGGMQYLD |
| | | | | | | | | | VWGQGTTVTVSS |
| 0317-A8 | Ab | SARS-CoV2 | | | SARS-CoV2 | S; S1 (non | B-cells; SARS-CoV2 | 71 | EVQLVQSGSELKKPGASVKVSCKASGYT |
| | | (weak) | | | | RBD) | Human Patient | | FTSYAMNWVRQAPGQGLEWMGWINT |
| | | | | | | | | | NTGNPTYAQGFTGRFVFSLDTSVSTAYL |
| | | | | | | | | | QISSLKAEDTAVYYCARAGPNYDFWSGY |
| | | | | | | | | | YQTFDYWGQGTMVTVSS |
| 0317-A9 | Ab | SARS-CoV2 | | | SARS-CoV2 | S; Link | B-cells; SARS-CoV2 | 72 | EVQLVQSGAEVKKPGASVKVSCKVSGYT |
| | | (weak) | | | | | Human Patient | | LTELSMHWVRQAPGKGLEWMGGFDP |
| | | | | | | | | | EDGETIYAQKFQGRVTMTEDTSTDTAY |
| | | | | | | | | | MELSSLRSEDTAVYYCATATAMDGYYYY |
| | | | | | | | | | YGMDVWGQGTTVTVSS |
| 0317-B1 | Ab | SARS-CoV2 | | | SARS-CoV2 | S; Link | B-cells; SARS-CoV2 | 73 | EVQLVESGAEVKKPGESLKISCKGSGYSF |
| | | (weak) | | | | | Human Patient | | TSYWIGWVRQMPGEGLEWMGIIYPGD |
| | | | | | | | | | SDTRYSPSFQGQVTISADKSISTAYLQWS |
| | | | | | | | | | SLKASDTAMYYCASAGSSWYGDAFDIW |
| | | | | | | | | | GQGTMVTVSS |
| 0317-C4 | Ab | SARS-CoV2 | | | SARS-CoV2 | S; Link | B-cells; SARS-CoV2 | 74 | QVQLVQSGAEVKKPGASVKVSCKVSGC |
| | | (weak) | | | | | Human Patient | | TLTELSMHWVRQAPGKGLEWMGGFD |
| | | | | | | | | | PEDGETIYAQKFQGRVTMTEDTSTDTAY |
| | | | | | | | | | MELSSLRSEDTAVYYCATATIFGVANNW |
| | | | | | | | | | FDPWGQGTTVTVSS |
| 0317-C9 | Ab | SARS-CoV2 | | | SARS-CoV2 | S; S2 | B-cells; SARS-CoV2 | 75 | QVQLVQSGGGVVQPGRSLRLSCAASGF |
| | | | | | | | Human Patient | | TFSSYGMHWVRQAPGKGLEWVAVISY |
| | | | | | | | | | DGSNKYYADSVKGRFTISRDNSKNTLYL |
| | | | | | | | | | QMNSLRAEDTAVYYCAKDLGYYDILTGQ |
| | | | | | | | | | LGGYYYYYGMDVWGQGTTVTVSS |
| 10C10 | Ab | SARS-CoV2 | | | SARS-CoV2 | S; link | B-cells; SARS-CoV2 | 76 | QVQLVQSGGGLVQPGGSLRLSCAASGF |
| | | | | | | | Human Patient | | TFSSYWMSWVRQAPGKGLEWVANIN |
| | | | | | | | | | QDGSEKYYVDSVKGRFTISRDNAKNSLY |
| | | | | | | | | | LQMNSLRAEDTAVYYCARDWDYDILTG |
| | | | | | | | | | SWFGAFDIWGQGTMVTVSS |
| 1A09 | Ab | SARS-CoV2 | SARS-CoV1 | | SARS-CoV2 | S; RBD | Immunised Mouse | 77 | QVQLKESGPGLVAPSQSLSITCTVSGFSL |
| | | | | | | | | | TSYAISWVRQPPGKGLEWLGVIWTGGG |
| | | | | | | | | | TNYNSALKSRLSISKDNSKSQVFLKMNSL |
| | | | | | | | | | QTDDTARYYCARKDYYYGSSYAMDYWG |
| | | | | | | | | | QGTSVTVSS |
| 1A10 | Ab | SARS-CoV2 | MERS-CoV, | | SARS-CoV2 | S; RBD | Immunised Mouse | 78 | EVQLQQSGAELVRPGASVKLSCKASGYT |
| | | | SARS-CoV1 | | | | | | FSDYYINWVKQRPGQGLEWIARIYPGS |
| | | | | | | | | | GNTYYNEKFTGKATLTAEKSSSTAYMQL |
| | | | | | | | | | SSLTSEDSAVYFCARDYGSSYVDYFDYW |
| | | | | | | | | | GQGTTLTVSS |
| 1A12 | Ab | SARS-CoV2 | MERS-CoV, | SARS-CoV2 | | S; RBD | Immunised Mouse | 79 | EVQLQQSGAELVRPGASVKLSCTASGFN |
| | | | SARS-CoV1 | (weak) | | | | | IKDDYMHWVKQRPEQGLEWIGWINPE |
| | | | | | | | | | NGDTEYASKFQGKAAITADTSSNTACLQ |
| | | | | | | | | | LSSLTSEDTAVYYCSTGGYGNYVDAMDY |
| | | | | | | | | | WGQGTSVTVSS |
| 1B07 | Ab | SARS-CoV2 | MERS-CoV, | SARS-CoV2 | | S; RBD | Immunised Mouse | 80 | QVQLKESGPGLVAPSQSLSITCTVSGFSL |
| | | | SARS-CoV1 | | | | | | TSYAISWVRQPPGKGLEWLGVIWTGGG |
| | | | | | | | | | TNYNSALKSRLSISKDNSKSQVFLKMNSL |
| | | | | | | | | | QTDDTARYYCARKDYYGSSSNVMDYW |
| | | | | | | | | | GQGTSVTVSS |
| 1610 | Ab | SARS-CoV2 | MERS-CoV, | SARS-CoV2 | | S; RBD | Immunised Mouse | 81 | QVQLKESGPGLVAPSQSLSITCTVSGFSL |
| | | | SARS-CoV1 | | | | | | TNYAINWVRQPPGKGLEWLGVIWTGG |
| | | | | | | | | | GTNYNSALRSRLSISKDNSKSQVFLKMN |
| | | | | | | | | | SLHTDDTARYYCARKDYYGSSLAMDYW |
| | | | | | | | | | GQGTSVTVSS |
| 1C05 | Ab | SARS-CoV1, | MERS-CoV | SARS-CoV2 | | S; RBD | Immunised Mouse | 82 | EVQLQQSGPELVKPGASVKISCKASGYS |
| | | SARS-CoV2 | | (weak) | | | | | FTDYYMNWVKQSPEKSLEWIGEINPST |
| | | | | | | | | | GGPTYNQKFKAKATVTVDKSSSTAYMQ |
| | | | | | | | | | LKSLTSEDSAVYYCARRNYDLYYYAMDY |
| | | | | | | | | | WGQGTSVTVSS |
| 1C06 | Ab | SARS-CoV2 | MERS-CoV, | | SARS-CoV2 | S; RBD | Immunised Mouse | 83 | EVQLQQSGAELAKPGASVKLSCKASGYT |
| | | | SARS-CoV1 | | | | | | FTNYWMHWVKQRPGQGLEWIGYINPS |
| | | | | | | | | | SGYTKFNQKFNDKATLTADKSSTTAYMQ |
| | | | | | | | | | LSSPTYEDSAVYYCARSDYYGSSYVGYA |
| | | | | | | | | | MDYWGQGTSVTVSS |
| 1C07 | Ab | SARS-CoV1, | MERS-CoV | | SARS-CoV2 | S; RBD | Immunised Mouse | 84 | EVQLQQPGPELVKPGASVKISCKASGYS |
| | | SARS-CoV2 | | | | | | | FTDYYMNWVKQSPEKSLEWIGEINPST |
| | | | | | | | | | GGTSYNQKFKGKATLTVDKSSSTAYMQL |
| | | | | | | | | | KSLTSEDSAVYYCARRNYDLYYYAMDYW |
| | | | | | | | | | GQGTSVTVSS |
| 1D04 | Ab | SARS-CoV2 | MERS-CoV, | | SARS-CoV2 | S; RBD | Immunised Mouse | 85 | EVQLQQSGAELVRPGASVKLSCTASGFN |
| | | | SARS-CoV1 | | | | | | IKDDYMHWVKQRPEQGLEWIGWINPE |
| | | | | | | | | | NGDTEYASKFQGKAAITADTSSNTACLQ |
| | | | | | | | | | LSSLTSEDTAVYYCSTGGYGNYVDAMDY |
| | | | | | | | | | WGQGTSVTVSS |
| 1D05 | Ab | SARS-CoV2 | MERS-CoV, | | SARS-CoV2 | S; RBD | Immunised Mouse | 86 | QVQLKESGPGLVAPSQSLSITCTVSGFSL |
| | | | SARS-CoV1 | | | | | | TSYAISWVRQPPGKGLEWLGVIWTGGG |
| | | | | | | | | | TNYNSALKSRLSISKDNSKSQVFLKMNSL |
| | | | | | | | | | QTDDTARYYCARKDYYGSSSNVMDYW |
| | | | | | | | | | GQGTSVTVSS |
| 1E02 | Ab | SARS-CoV2 | MERS-CoV, | | SARS-CoV2 | S; RBD | Immunised Mouse | 87 | EVQLQQPGPELVKPGASVKISCKASGYS |
| | | | SARS-CoV1 | | | | | | FTDYYMNWVKQSPEKSLEWIGEINPST |
| | | | | | | | | | GGTSYNQKFKGKATLTVDKSSSTAYMQL |
| | | | | | | | | | KSLTSEDSAVYYCSRRNYDLYYYAMDYW |
| | | | | | | | | | GQGTSVTVSS |
| 1E07 | Ab | SARS-CoV2 | MERS-CoV, | SARS-CoV2 | | S; RBD | Immunised Mouse | 88 | EVQLQQSGPELVKPGASVKISCKASGYS |
| | | | SARS-CoV1 | | | | | | FTGYSMNWVKKSPEKSLEWIGEINPSTG |
| | | | | | | | | | GTTYNQKFKAKATLTVDKSSSTAYIQLKS |
| | | | | | | | | | LTSEDSAVYYCARGAGAYWGQGTLVTV |
| | | | | | | | | | SA |
| 1E10 | Ab | SARS-CoV2 | MERS-CoV, | | SARS-CoV2 | S; RBD | Immunised Mouse | 89 | EVQLQQSGPELVKPGASVKISCKASGYA |
| | | | SARS-CoV1 | | | | | | FSSSWMNWVKQRPGKGLEWIGRIYPG |
| | | | | | | | | | DGDTNYNGKFKGKATLTADKSSSTAYM |
| | | | | | | | | | QLSSLTSEDSAVYFCARDHGPAWFAYRG |
| | | | | | | | | | QGTLVTVSA |
| 1H06 | Ab | SARS-CoV2 | MERS-CoV, | | SARS-CoV2 | S; RBD | Immunised Mouse | 90 | EVQLQQSGPELVKPGASVKISCKASGYS |
| | | | SARS-CoV1 | | | | | | FTGYSMNWVKKSPEKSLEWIGEINPSTG |
| | | | | | | | | | GTTYNQKFKAKATLTVDKSSSTAYIQLKS |
| | | | | | | | | | LTSEDSAVYYCARGAGAYWGQGTLVTV |
| | | | | | | | | | SA |
| 1H10 | Ab | SARS-CoV2 | MERS-CoV, | SARS-CoV2 | | S; RBD | Immunised Mouse | 91 | QVQLKESGPGLVAPSQSLSITCTVSGFSL |
| | | | SARS-CoV1 | (weak) | | | | | TNYAISWVRQPPGKGLEWLGVIWTGG |
| | | | | | | | | | GTNYNSALKSRLSISKDNSKSQVFLKMN |
| | | | | | | | | | SLQTDDTARYYCARISYYDYEGVDYWGQ |
| | | | | | | | | | GTTLTVSS |
| 1M-1D2 | Ab | SARS-CoV2 | | SARS-CoV2 | | S; RBD | B-cells; SARS-CoV2 | 92 | QVQLVESGGGLVQPGGSLRLSCAASGF |
| | | | | | | | Human Patient | | TFSSYAMHWVRQAPGKGLEYVSAISGN |
| | | | | | | | | | GGSTYYAKSVKGRFTISRDNSKNTLYLQ |
| | | | | | | | | | MGSLRAEDMAVYYCARGAEYYDFWSG |
| | | | | | | | | | YYSAYFDYWGQGTLVTVSS |
| 2B04 | Ab | SARS-CoV2 | MERS-CoV, | SARS-CoV2 | | S; RBD | Immunised Mouse | 93 | QVQLKQSGPGLVAPSQSLSITCTVSGFSL |
| | | | SARS-CoV1 | | | | | | INYAISWVRQPPGKGLEWLGVIWTGGG |
| | | | | | | | | | TNYNSALKSRLSISKDNSKSQVFLKMNSL |
| | | | | | | | | | QTDDTARYYCARKDYYGRYYGMDYWG |
| | | | | | | | | | QGTSVTVSS |
| 2C02 | Ab | SARS-CoV1, | MERS-CoV | | SARS-CoV2 | S; RBD | Immunised Mouse | 94 | EVQLQQPGAELVKPGASVKVSCKASGYI |
| | | SARS-CoV2 | | | | | | | FTNYWMHWVKQRPGQGLEWIGRIHPS |
| | | | | | | | | | DSDTKYNQKFKGKATLTVDKSSSTAYMQ |
| | | | | | | | | | LSSLTSEDSAVYYCAILDSYWYFDVWGT |
| | | | | | | | | | GTTVTVSS |
| 2C03 | Ab | SARS-CoV2 | MERS-CoV, | SARS-CoV2 | | S; RBD | Immunised Mouse | 95 | EVQLQQSGPELVKPGASVKISCKASGYA |
| | | | SARS-CoV1 | (weak) | | | | | FSSSWMNWVKQRPGKGLEWIGRIYPG |
| | | | | | | | | | DGDTNYNGKLKGKATLTADKSSSTAYM |
| | | | | | | | | | QLSSLTSEDSAVYFCARKSYGYWHFDV |
| | | | | | | | | | WGTGTTVTVSS |
| 2C04 | Ab | SARS-CoV2 | MERS-CoV, | | SARS-CoV2 | S; RBD | Immunised Mouse | 96 | EVQLQQPGPELVKPGTSVKISCKASGYT |
| | | | SARS-CoV1 | | | | | | FTDYYMNWVKQSHGKSLEWIGDINPN |
| | | | | | | | | | NGGTNYNQKFKGKATLTVDKSSSTAYM |
| | | | | | | | | | ELRSLTSEDSAVCYCAAGKGDYWGQGT |
| | | | | | | | | | TLTVSS |
| 2D01 | Ab | SARS-CoV2 | MERS-CoV, | | SARS-CoV2 | S; RBD | Immunised Mouse | 97 | EVQLQQPGAELVKPGASVKVSCKASGYI |
| | | | SARS-CoV1 | | | | | | FTNYWMHWVKQRPGQGLEWIGRIHPS |
| | | | | | | | | | DSDTKYNQKFKGKATLTVDKSSSTAYMQ |
| | | | | | | | | | LSSLTSEDSAVYYCAILDSYWYFDVWGT |
| | | | | | | | | | GTTVTVSS |
| 2D08 | Ab | SARS-CoV2 | MERS-CoV, | SARS-CoV2 | | S; RBD | Immunised Mouse | 98 | EVQLQQSGPELVKPGTSVKISCKASGYTF |
| | | | SARS-CoV1 | (weak) | | | | | TDYYMNWVKQSHGKSLEWIGDINPNN |
| | | | | | | | | | GGTNYNQKFKGKATLTVDKSSSTAYMEL |
| | | | | | | | | | RSLTSEDSAVYYCAAGKGDYWGQGTTL |
| | | | | | | | | | TVSS |
| 2D11 | Ab | SARS-CoV2 | MERS-CoV, | | SARS-CoV2 | S; RBD | Immunised Mouse | 99 | EVQLQQSGPELVKPGASVKISCKASGYT |
| | | | SARS-CoV1 | | | | | | FTDYYMNWVKQSHGKSLEWIGDINPN |
| | | | | | | | | | NGGTSYNQKFKGKATLTVDKSSSTAYME |
| | | | | | | | | | LRSLTSEDSAVYYCARKGDGYYGGFAYW |
| | | | | | | | | | GQGTLVTVSA |
| 2E06 | Ab | SARS-CoV1, | MERS-CoV | SARS-CoV2 | | S; RBD | Immunised Mouse | 100 | EVQLQQSVADLVRPGASVKLSCTASGFN |
| | | SARS-CoV2 | | (weak) | | | | | IKNTYMHWVKQRPEQGLEWIGRIDPTN |
| | | | | | | | | | GDTKYVSKFQGKATITADTSSNTAYLQLS |
| | | | | | | | | | SLTSEDTAIYYCATYGSYYLYYYAMNYWG |
| | | | | | | | | | QGTSVTVSS |
| 2E10 | Ab | SARS-CoV1, | MERS-CoV | | SARS-CoV2 | S; RBD | Immunised Mouse | 101 | EVQLQQPGAELVRPGASVKLSCKASGYT |
| | | SARS-CoV2 | | | | | | | FTDYYINWVKQRPGQGLEWIARIYPGT |
| | | | | | | | | | GTTYYNEKFKGKATLTAEKSSSTAYMQLS |
| | | | | | | | | | SLTSEDSAVYFCARYDGNLYYYAMDYW |
| | | | | | | | | | GQGTSVTVSS |
| 2F04 | Ab | SARS-CoV2 | MERS-CoV, | | SARS-CoV2 | S; RBD | Immunised Mouse | 102 | EVQLQQPGAELVKPGASVKMSCKASGY |
| | | | SARS-CoV1 | | | | | | TFTSYWITWVKQRPGQGLEWIGDIYPG |
| | | | | | | | | | SGSTKYNEKFRSEATLTVDTSSTTAYMQL |
| | | | | | | | | | SSLTSEDSAVYYCARWDFYGSRTFDYWG |
| | | | | | | | | | QGTTLTVSS |
| 2H04 | Ab | SARS-CoV2 | MERS-CoV, | SARS-CoV2 | | S; RBD | Immunised Mouse | 103 | EVQLQQSGAELVKPGASVKMSCKASGY |
| | | | SARS-CoV1 | | | | | | TFTSYWITWVKQRPGQGLEWIGDIYPG |
| | | | | | | | | | SGSTKYNEKFRSEATLTVDTSSTTAYMQL |
| | | | | | | | | | SSLTSEDSAVYYCARWDFYGSRTFDYWG |
| | | | | | | | | | QGTTLTVSS |
| 2M- | Ab | SARS-CoV2 | | | SARS-CoV2 | S; RBD | B-cells; SARS-CoV2 | 104 | EVQLVESGGGLVQPGGSLRLSCAASGFT |
| 10B11 | | | | | | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAEDTAVYYCARATWLRGVMDVW |
| | | | | | | | | | GQGTTVTVSS |
| 2M-12D7 | Ab | SARS-CoV2 | | | SARS-CoV2 | S; S2 | B-cells; SARS-CoV2 | 105 | EVQLVESGGGLVQPGRSLRLSCAASGFT |
| | | | | | | | Human Patient | | FDDYVMHWVRQAPGKGLEWVSGINW |
| | | | | | | | | | NSGSIGYADSVKGRFTISRDNAKNSLYLQ |
| | | | | | | | | | MNSLRAEDTALYYCAKDVRYCSSTSCYFS |
| | | | | | | | | | AFDIWGQGTMVTVSS |
| 2M-13A3 | Ab | SARS-CoV2 | | | SARS-CoV2 | S; S2 | B-cells; SARS-CoV2 | 106 | EVQLVESGGGVVQPGRSLRLSCAASGFT |
| | | | | | | | Human Patient | | FSGYAMHWVRQAPGKGLEWVAVISYD |
| | | | | | | | | | GSNKYYADSVKGRFTISRDNSKNTLYLQ |
| | | | | | | | | | MNSLRAEDTAVYYCARGGGSYYYWFDP |
| | | | | | | | | | WGQGTMVTVSS |
| 2M- | Ab | SARS-CoV2 | | | SARS-CoV2 | S; S2 | B-cells; SARS-CoV2 | 107 | EVQLVQWGAGLLKPSETLSLTCAVYGGS |
| 13D11 | | | | | | | Human Patient | | FSGYYWSWIRQPPGKGLEWIGEINHSG |
| | | | | | | | | | STNYNPSLKSRVTISVDTSKNQFSLKLSS |
| | | | | | | | | | VTAADTAVYYCARAGYSSSWYGVRGVDP |
| | | | | | | | | | WGQGTMVTVSS |
| 2M-14B2 | Ab | SARS-CoV2 | | | SARS-CoV2 | S; S2 | B-cells; SARS-CoV2 | 108 | QVQLLQSGGGVVQPGRSLRLSCAASGF |
| | | | | | | | Human Patient | | TFSTYGMHWVRQAPGKGLEWVAVISY |
| | | | | | | | | | DGSNKYYADSVKGRFTISRDNSKNTLYL |
| | | | | | | | | | QMNSLRAEDTAVYYCAKGSDIVVVPVG |
| | | | | | | | | | NWFDPWGQGTLVTVSS |
| 2M-14E4 | Ab | SARS-CoV2 | | | SARS-CoV2 | S; S2 | B-cells; SARS-CoV2 | 109 | QVQLQESGPGLVKPSETLSLACTVSGGS |
| | | | | | | | Human Patient | | VSSDNSYWSWIRQPPGKGLEWIGYTFH |
| | | | | | | | | | SGSANYNPSLKSRVTISVDTSKNQFSLKL |
| | | | | | | | | | SSVTAADTAVYYCARVQRYYPDSSGFYG |
| | | | | | | | | | RRFDIWGQGTLVTVSS |
| 2M-14E5 | Ab | SARS-CoV2 | | | SARS-CoV2 | S; S2 | B-cells; SARS-CoV2 | 110 | EVQLVESGGGVVQPGRSLRLSCAASGFT |
| | | | | | | | Human Patient | | FSTYSMHWVRQAPGKGLEWVAVISYD |
| | | | | | | | | | GSNKYYADSVKGRFTISRDNSKNTLYLQ |
| | | | | | | | | | VNSLRAEDTAVYYCARSGGGSYRGPFDY |
| | | | | | | | | | WGQGTLVTVSS |
| 2M-2D1 | Ab | SARS-CoV2 | | | SARS-CoV2 | S; S2 | B-cells; SARS-COV2 | 111 | EVQLVESGPGLVKPSETLSLTCTVSGGSI |
| | | | | | | | Human Patient | | SSSSYYWGWIRQPPGKGLEWIGSIYYSG |
| | | | | | | | | | STYYNPSLKSRVTISVDTSKNQFSLKLSS |
| | | | | | | | | | VTAADTAVYYSARGDRIQLWLLDAFDIW |
| | | | | | | | | | GQGTMVTVSS |
| 2M-2D4 | Ab | SARS-CoV2 | | | SARS-CoV2 | S; S2 | B-cells; SARS-CoV2 | 112 | EVQLVESGGGLVQPGGSLRLSCAASGFT |
| | | | | | | | Human Patient | | FSSYAMSWVRQAPGKGLEWVSAISGSG |
| | | | | | | | | | GSTYYADSVKGRFTISRDNSKNTLYLQM |
| | | | | | | | | | NSLRAEDTAVYYCAKIGLGLGGLLRRYFD |
| | | | | | | | | | YWGQGTLVTVSS |
| 2M-2G12 | Ab | SARS-CoV2 | | | SARS-CoV2 | S; S2 | B-cells; SARS-CoV2 | 113 | QVQLVESGGGLLKPGETLSLSCAASGFTF |
| | | | | | | | Human Patient | | SDYYWSWIRQAPGKGLEWIAVINHSGS |
| | | | | | | | | | TIYYPYPVKGVFIFSRDTANNFSLKLMN |
| | | | | | | | | | MMTSDTAVYYCGTRIMITWYSRRGMD |
| | | | | | | | | | GWGKGVTVTVSS |
| 2M-4G4 | Ab | SARS-CoV2 | | | SARS-CoV2 | S; RBD | B-cells; SARS-CoV2 | 114 | EVQLVESGAEVKKPGASVKVSCKASGYT |
| | | | | | | | Human Patient | | FTSYYIHWVRQAPGQGLEWMGVINPS |
| | | | | | | | | | GGSTTYAQKFQGRVTVTRDTSTSTVYM |
| | | | | | | | | | ELSSLRSEDTAVYYCARERGDSSGYYEII |
| | | | | | | | | | TTANRRFGMDVWGQGTTVTVSS |
| 2M-7E9 | Ab | SARS-CoV2 | | | SARS-CoV2 | S; S2 | B-cells; SARS-CoV2 | 115 | EVQLVESGAEVKKPGSSVKVSCKASGGT |
| | | | | | | | Human Patient | | FSSFAISWVRQAPGQGLEWMGGIIPIFD |
| | | | | | | | | | TTNYAQKFQGRVTITADESTRTAYMELS |
| | | | | | | | | | SLRSEDTAVYYCARIPGWDRGTDRNWN |
| | | | | | | | | | DDWGQGTTVTVSS |
| 2M-8E7 | Ab | SARS-CoV2 | | | SARS-CoV2 | S; S2 | B-cells; SARS-CoV2 | 116 | EVQLVESGAEVKKPGSSVKVSCKASGGT |
| | | | | | | | Human Patient | | FSSYAISWVRQAPGQGLEWMGGIIPIFG |
| | | | | | | | | | TANYAQKFQGRVTVTADESTSTAYMELS |
| | | | | | | | | | SLRSEDTAVYYCARTYSFDSSGYYYDYW |
| | | | | | | | | | GQGTMVTVSS |
| 2M-8H10 | Ab | SARS-CoV2 | | | SARS-CoV2 | S; S2 | B-cells; SARS-CoV2 | 117 | QVQLVESGGGVVLPGRSLRLSCAASGFT |
| | | | | | | | Human Patient | | FSTFAMHWVRQAPGKGLEWVAVISDE |
| | | | | | | | | | GSNKYYADSVKGRFTISRDNSRNTLYLQ |
| | | | | | | | | | MNSLRAEDTAVYYCARAFYDSNWSVGS |
| | | | | | | | | | YFDSWGQGTPVTVSS |
| 2M-9F10 | Ab | SARS-CoV2 | | | SARS-CoV2 | S; S2 | B-cells; SARS-CoV2 | 118 | EVQLLQSGGDLIQPGRSLRLSCAASGFSF |
| | | | | | | | Human Patient | | EDYAMHWVRQAPGQGLEWVSGISYN |
| | | | | | | | | | GGSIDYVDSVKGRFTISRDNAKNALYLE |
| | | | | | | | | | MNSLRPEDTAFYYCAKDSVRREYTHARV |
| | | | | | | | | | PFDNWGLGTLVTVSS |
| 2M-9H1 | Ab | SARS-CoV2 | | | SARS-CoV2 | S; S2 | B-cells; SARS-CoV2 | 119 | EVQLLESGGGVVQPGRSLRLSCVASGFN |
| | | | | | | | Human Patient | | FNNYGMHWVRQAPGKGLEWLAALSYE |
| | | | | | | | | | GSKEHYADSLKGRFTVSRDYSRATLHLH |
| | | | | | | | | | MNSLEPEDTAVYFCAKSSKIFYLGESREV |
| | | | | | | | | | DYWGRGTLVTVSS |
| 31B5 | Ab | SARS-CoV2 | | SARS-CoV2 | | S; RBD | B-cells; SARS-CoV2 | | ND |
| | | | | | | | Human Patient | | |
| 31B9 | Ab | SARS-CoV2 | | | SARS-CoV2 | S; RBD | B-cells; SARS-CoV2 | | ND |
| | | | | | | | Human Patient | | |
| 32D4 | Ab | SARS-CoV2 | | SARS-CoV2 | | S; RBD | B-cells; SARS-CoV2 | | ND |
| | | | | | | | Human Patient | | |
| 413-2 | Ab | SARS-CoV2 | SARS-CoV1 | SARS-CoV2 | SARS-CoV1 | S; non- | B-cells; SARS-CoV2 | | ND |
| | | | | | | RBD | Human Patient | | |
| 414-1 | Ab | SARS-CoV2 | SARS-CoV1 | SARS-CoV2 | SARS-CoV1 | S; RBD | B-cells; SARS-CoV2 | | ND |
| | | | | | | | Human Patient | | |
| 47D11 | Ab | SARS-CoV1, | | SARS-CoV2 | | S; RBD | B-cells; SARS-CoV1 | | ND |
| | | SARS-CoV2 | | and SARS- | | | Human Patient | | |
| | | | | CoV1 | | | | | |
| 4A8 | Ab | SARS-CoV2 | | SARS-CoV2 | | S; S1 (non | B-cells; SARS-CoV2 | 120 | EVQLVESGAEVKKPGASVKVSCKVSGYT |
| | | | | | | RBD) | Human Patient | | LTELSMHWVRQAPGKGLEWMGGFDP |
| | | | | | | | | | EDGETMYAQKFQGRVTMTEDTSTDTAY |
| | | | | | | | | | MELSSLRSEDTAVYYCATSTAVAGTPDLF |
| | | | | | | | | | DYYYGMDVWGQGTTVTVSS |
| 505-3 | Ab | SARS-CoV2 | SARS-CoV1 | SARS-CoV2 | SARS-CoV1 | S; RBD | B-cells; SARS-CoV2 | | ND |
| | | | | | | | Human Patient | | |
| 505-5 | Ab | SARS-CoV2 | SARS-CoV1 | SARS-CoV2 | SARS-CoV1 | S; RBD | B-cells; SARS-CoV2 | | ND |
| | | | | | | | Human Patient | | |
| 505-8 | Ab | SARS-CoV2 | SARS-CoV1 | SARS-CoV2 | SARS-CoV1 | S; non- | B-cells; SARS-CoV2 | | ND |
| | | | | | | RBD | Human Patient | | |
| 515-1 | Ab | SARS-CoV2 | SARS-CoV1 | SARS-CoV2 | SARS-CoV1 | S; RBD | B-cells; SARS-CoV2 | | ND |
| | | | | | | | Human Patient | | |
| 515-5 | Ab | SARS-CoV2 | SARS-CoV1 | SARS-CoV2 | SARS-CoV1 | S; non- | B-cells; SARS-CoV2 | | ND |
| | | | | | | RBD | Human Patient | | |
| 553-15 | Ab | SARS-CoV1, | | SARS-CoV1, | | S; RBD | B-cells; SARS-CoV2 | | ND |
| | | SARS-CoV2 | | SARS-CoV2 | | | Human Patient | | |
| 553-49 | Ab | SARS-CoV2 | SARS-CoV1 | SARS-CoV2 | SARS-CoV1 | S; RBD | B-cells; SARS-CoV2 | | ND |
| | | | | | | | Human Patient | | |
| 553-60 | Ab | SARS-CoV2 | SARS-CoV1 | SARS-CoV2 | SARS-CoV1 | S; RBD | B-cells; SARS-CoV2 | | ND |
| | | | | | | | Human Patient | | |
| 553-63 | Ab | SARS-CoV2 | SARS-CoV1 | SARS-CoV2 | SARS-CoV1 | S; RBD | B-cells; SARS-CoV2 | | ND |
| | | | | | | | Human Patient | | |
| 8D2 | Ab | SARS-CoV2 | | | SARS-CoV2 | S; S2 | B-cells; SARS-CoV2 | 121 | EVQLVESGGGLVQPGGSLRLSCAASGFT |
| | | | | | | | Human Patient | | FSSYWMSWVRQAPGKGLEWVANINQ |
| | | | | | | | | | DGSEKYYVDSVKGRFTISRDNAKNSLYL |
| | | | | | | | | | QVNSLRAEDTAVYYCARDWDYDILTGS |
| | | | | | | | | | WFGAFDIWGQGTTVTVSS |
| 8D9 | Ab | SARS-CoV2 | | | SARS-CoV2 | S; S2 | B-cells; SARS-CoV2 | 122 | EVQLLESGGGLVQPGGSLRLSCTASGFT |
| | | | | | | | Human Patient | | FSSYWMSWVRQAPGKGLEWVANIQQ |
| | | | | | | | | | DGSEKYYVDSVKGRFTISRDNAKNSLYL |
| | | | | | | | | | QMNSLRAEDTAVYYCARPTIGYSYGSDY |
| | | | | | | | | | WGQGTTVTVSS |
| 9A1 | Ab | SARS-CoV2 | | | SARS-CoV2 | S; S2 | B-cells; SARS-CoV2 | 123 | EVQLLESGGGVVQPGRSLRLSCVVSGFT |
| | | | | | | | Human Patient | | FNNYGMHWVRQAPGKGLEWVAVISYE |
| | | | | | | | | | GSVKYYGDHVDGRFTISRDPFKNTLYLH |
| | | | | | | | | | MNNLRPDDTAVYYCAKVSAIFWLGQGL |
| | | | | | | | | | SPIDVWGQGTTVTVSS |
| Ab_510A | Ab | SARS-CoV2 | | SARS-CoV2 | | S; RBD | B-cells; SARS-CoV2 | | ND |
| | | | | (weak) | | | Human Patient | | |
| Ab_510A5 | Ab | SARS-CoV2 | | | SARS-CoV2 | S; RBD | B-cells; SARS-CoV2 | | ND |
| | | | | | | | Human Patient | | |
| Ab_510D7 | Ab | SARS-CoV2 | | SARS-CoV2 | | S; RBD | B-cells; SARS-CoV2 | | ND |
| | | | | | | | Human Patient | | |
| Ab_510G | Ab | SARS-CoV2 | | SARS-CoV2 | | S; RBD | B-cells; SARS-CoV2 | | ND |
| | | | | | | | Human Patient | | |
| Ab_510H10 | Ab | SARS-CoV2 | | | | S; RBD | B-cells; SARS-CoV2 | | ND |
| | | | | | | | Human Patient | | |
| Ab_510H2 | Ab | SARS-CoV2 | | SARS-CoV2 | | S; RBD | B-cells; SARS-CoV2 | | ND |
| | | | | (weak) | | | Human Patient | | |
| Ab_510H | Ab | SARS-CoV2 | | SARS-CoV2 | | S; RBD | B-cells; SARS-CoV2 | | ND |
| | | | | | | | Human Patient | | |
| Ab_510H7 | Ab | SARS-CoV2 | | SARS-CoV2 | | S; RBD | B-cells; SARS-CoV2 | | ND |
| | | | | | | | Human Patient | | |
| Ab_511A1 | Ab | SARS-CoV2 | | | | S; RBD | B-cells; SARS-CoV2 | | ND |
| | | | | | | | Human Patient | | |
| Ab_511A5 | Ab | SARS-CoV2 | | SARS-CoV2 | | S; RBD | B-cells; SARS-CoV2 | | ND |
| | | | | | | | Human Patient | | |
| Ab_511B11 | Ab | SARS-CoV2 | | | | S; RBD | B-cells; SARS-CoV2 | | ND |
| | | | | | | | Human Patient | | |
| Ab_511B | Ab | SARS-CoV2 | | SARS-CoV2 | | S; RBD | B-cells; SARS-CoV2 | | ND |
| | | | | | | | Human Patient | | |
| Ab_511D11 | Ab | SARS-CoV2 | | | | S; RBD | B-cells; SARS-CoV2 | | ND |
| | | | | | | | Human Patient | | |
| Ab_511E5 | Ab | SARS-CoV2 | | | | S; RBD | B-cells; SARS-CoV2 | | ND |
| | | | | | | | Human Patient | | |
| Ab_511E7 | Ab | SARS-CoV2 | | | | S; RBD | B-cells; SARS-CoV2 | | ND |
| | | | | | | | Human Patient | | |
| Ab_511E9 | Ab | SARS-CoV2 | | | | S; RBD | B-cells; SARS-CoV2 | | ND |
| | | | | | | | Human Patient | | |
| Ab_511G5 | Ab | SARS-CoV2 | | | | S; RBD | B-cells; SARS-CoV2 | | ND |
| | | | | | | | Human Patient | | |
| Ab_511G7 | Ab | SARS-CoV2 | | SARS-CoV2 | | S; RBD | B-cells; SARS-CoV2 | | ND |
| | | | | | | | Human Patient | | |
| Ab_511H11 | Ab | SARS-CoV2 | | | | S; RBD | B-cells; SARS-CoV2 | | ND |
| | | | | | | | Human Patient | | |
| Ab_511H7 | Ab | SARS-CoV2 | | | | S; RBD | B-cells; SARS-CoV2 | | ND |
| | | | | | | | Human Patient | | |
| Ab_51A1 | Ab | SARS-CoV2 | | SARS-CoV2 | | S; RBD | B-cells; SARS-CoV2 | | ND |
| | | | | (weak) | | | Human Patient | | |
| Ab_51A3 | Ab | SARS-CoV2 | | | | S; RBD | B-cells; SARS-CoV2 | | ND |
| | | | | | | | Human Patient | | |
| Ab_51D2 | Ab | SARS-CoV2 | | SARS-CoV2 | | S; RBD | B-cells; SARS-CoV2 | | ND |
| | | | | | | | Human Patient | | |
| Ab_51D3 | Ab | SARS-CoV2 | | SARS-CoV2 | | S; RBD | B-cells; SARS-CoV2 | | ND |
| | | | | (weak) | | | Human Patient | | |
| Ab_51D4 | Ab | SARS-CoV2 | | SARS-CoV2 | | S; RBD | B-cells; SARS-CoV2 | | ND |
| | | | | (weak) | | | Human Patient | | |
| Ab_51D7 | Ab | SARS-CoV2 | | SARS-CoV2 | | S; RBD | B-cells; SARS-CoV2 | | ND |
| | | | | (weak) | | | Human Patient | | |
| Ab_51E10 | Ab | SARS-CoV2 | | SARS-CoV2 | | S; RBD | B-cells; SARS-CoV2 | | ND |
| | | | | (weak) | | | Human Patient | | |
| Ab_51E12 | Ab | SARS-CoV2 | | SARS-CoV2 | | S; RBD | B-cells; SARS-CoV2 | | ND |
| | | | | | | | Human Patient | | |
| Ab_51E7 | Ab | SARS-CoV2 | | SARS-CoV2 | | S; RBD | B-cells; SARS-CoV2 | | ND |
| | | | | | | | Human Patient | | |
| Ab_51F11 | Ab | SARS-CoV2 | | SARS-CoV2 | | S; RBD | B-cells; SARS-CoV2 | | ND |
| | | | | | | | Human Patient | | |
| Ab_52C1 | Ab | SARS-CoV2 | | | | S; RBD | B-cells; SARS-CoV2 | | ND |
| | | | | | | | Human Patient | | |
| Ab_52C6 | Ab | SARS-CoV2 | | SARS-CoV2 | | S; RBD | B-cells; SARS-CoV2 | | ND |
| | | | | | | | Human Patient | | |
| Ab_52F7 | Ab | SARS-CoV2 | | SARS-CoV2 | | S; RBD | B-cells; SARS-CoV2 | | ND |
| | | | | (weak) | | | Human Patient | | |
| Ab_52G9 | Ab | SARS-CoV2 | | SARS-CoV2 | | S; RBD | B-cells; SARS-CoV2 | | ND |
| | | | | | | | Human Patient | | |
| Ab_53C10 | Ab | SARS-CoV2 | | | | S; RBD | B-cells; SARS-CoV2 | | ND |
| | | | | | | | Human Patient | | |
| Ab_53C5 | Ab | SARS-CoV2 | | | | S; RBD | B-cells; SARS-COV2 | | ND |
| | | | | | | | Human Patient | | |
| Ab_53F12 | Ab | SARS-CoV2 | | SARS-CoV2 | | S; RBD | B-cells; SARS-CoV2 | | ND |
| | | | | (weak) | | | Human Patient | | |
| Ab_53F9 | Ab | SARS-CoV2 | | | | S; RBD | B-cells; SARS-CoV2 | | ND |
| | | | | | | | Human Patient | | |
| Ab_53H3 | Ab | SARS-CoV2 | | SARS-CoV2 | | S; RBD | B-cells; SARS-CoV2 | | ND |
| | | | | (weak) | | | Human Patient | | |
| Ab_55A8 | Ab | SARS-CoV2 | | SARS-CoV2 | | S; RBD | B-cells; SARS-CoV2 | | ND |
| | | | | (weak) | | | Human Patient | | |
| Ab_55C9 | Ab | SARS-CoV2 | | SARS-CoV2 | | S; RBD | B-cells; SARS-CoV2 | | ND |
| | | | | (weak) | | | Human Patient | | |
| Ab_56C12 | Ab | SARS-CoV2 | | SARS-CoV2 | | S; RBD | B-cells; SARS-CoV2 | | ND |
| | | | | | | | Human Patient | | |
| Ab_56D7 | Ab | SARS-CoV2 | | SARS-CoV2 | | S; RBD | B-cells; SARS-CoV2 | | ND |
| | | | | (weak) | | | Human Patient | | |
| Ab_56E1 | Ab | SARS-CoV2 | | SARS-CoV2 | | S; RBD | B-cells; SARS-CoV2 | | ND |
| | | | | | | | Human Patient | | |
| Ab_56H11 | Ab | SARS-CoV2 | | | | S; RBD | B-cells; SARS-CoV2 | | ND |
| | | | | | | | Human Patient | | |
| Ab_56H3 | Ab | SARS-CoV2 | | SARS-CoV2 | | S; RBD | B-cells; SARS-CoV2 | | ND |
| | | | | | | | Human Patient | | |
| Ab_57A6 | Ab | SARS-CoV2 | | SARS-CoV2 | | S; RBD | B-cells; SARS-CoV2 | | ND |
| | | | | | | | Human Patient | | |
| Ab_57A8 | Ab | SARS-CoV2 | | SARS-CoV2 | | S; RBD | B-cells; SARS-CoV2 | | ND |
| | | | | | | | Human Patient | | |
| Ab_57A9 | Ab | SARS-CoV2 | | SARS-CoV2 | | S; RBD | B-cells; SARS-CoV2 | | ND |
| | | | | | | | Human Patient | | |
| Ab_57B8 | Ab | SARS-CoV2 | | SARS-CoV2 | | S; RBD | B-cells; SARS-CoV2 | | ND |
| | | | | (weak) | | | Human Patient | | |
| Ab_57C4 | Ab | SARS-CoV2 | | SARS-CoV2 | | S; RBD | B-cells; SARS-CoV2 | | ND |
| | | | | | | | Human Patient | | |
| Ab_57E11 | Ab | SARS-CoV2 | | SARS-CoV2 | | S; RBD | B-cells; SARS-CoV2 | | ND |
| | | | | | | | Human Patient | | |
| Ab_57F7 | Ab | SARS-CoV2 | | SARS-CoV2 | | S; RBD | B-cells; SARS-CoV2 | | ND |
| | | | | (weak) | | | Human Patient | | |
| Ab_57G9 | Ab | SARS-CoV2 | | SARS-CoV2 | | S; RBD | B-cells; SARS-CoV2 | | ND |
| | | | | | | | Human Patient | | |
| Ab_58A4 | Ab | SARS-CoV2 | | | | S; RBD | B-cells; SARS-CoV2 | | ND |
| | | | | | | | Human Patient | | |
| Ab_58D2 | Ab | SARS-CoV2 | | | | S; RBD | B-cells; SARS-CoV2 | | ND |
| | | | | | | | Human Patient | | |
| Ab_58G1 | Ab | SARS-CoV2 | | SARS-CoV2 | | S; RBD | B-cells; SARS-CoV2 | | ND |
| | | | | | | | Human Patient | | |
| Ab_58G6 | Ab | SARS-CoV2 | | | SARS-CoV2 | S; RBD | B-cells; SARS-CoV2 | | ND |
| | | | | | | | Human Patient | | |
| Ab_59A2 | Ab | SARS-CoV2 | | | | S; RBD | B-cells; SARS-CoV2 | | ND |
| | | | | | | | Human Patient | | |
| Ab_59D6 | Ab | SARS-CoV2 | | | | S; RBD | B-cells; SARS-CoV2 | | ND |
| | | | | | | | Human Patient | | |
| Ab_81A11 | Ab | SARS-CoV2 | | | | S; RBD | B-cells; SARS-CoV2 | | ND |
| | | | | | | | Human Patient | | |
| Ab_81C3 | Ab | SARS-CoV2 | | SARS-CoV2 | | S; RBD | B-cells; SARS-CoV2 | | ND |
| | | | | (weak) | | | Human Patient | | |
| Ab_81C7 | Ab | SARS-CoV2 | | | | S; RBD | B-cells; SARS-CoV2 | | ND |
| | | | | | | | Human Patient | | |
| Ab_81C8 | Ab | SARS-CoV2 | | SARS-CoV2 | | S; RBD | B-cells; SARS-CoV2 | | ND |
| | | | | | | | Human Patient | | |
| Ab_81E1 | Ab | SARS-CoV2 | | SARS-CoV2 | | S; RBD | B-cells; SARS-CoV2 | | ND |
| | | | | (weak) | | | Human Patient | | |
| Ab_81E10 | Ab | SARS-CoV2 | | | | S; RBD | B-cells; SARS-CoV2 | | ND |
| | | | | | | | Human Patient | | |
| Ab_81F2 | Ab | SARS-CoV2 | | | | S; RBD | B-cells; SARS-CoV2 | | ND |
| | | | | | | | Human Patient | | |
| Ab_82B6 | Ab | SARS-CoV2 | | | | S; RBD | B-cells; SARS-CoV2 | | ND |
| | | | | | | | Human Patient | | |
| Ab_82C6 | Ab | SARS-CoV2 | | | | S; RBD | B-cells; SARS-CoV2 | | ND |
| | | | | | | | Human Patient | | |
| Ab_82F6 | Ab | SARS-CoV2 | | SARS-CoV2 | | S; RBD | B-cells; SARS-CoV2 | | ND |
| | | | | | | | Human Patient | | |
| Acharya | Ab | HIV | | | | S Glycans | B-cells; HIV Human | | Various |
| et al., | | Glycans, | | | | | Patient | | |
| 2020 | | SARS-CoV2 | | | | | | | |
| | | Glycans | | | | | | | |
| B38 | Ab | SARS-CoV2 | | SARS-CoV2 | | S; RBD | B-cells; SARS-CoV2 | 124 | EVQLVESGGGLVQPGGSLRLSCAASGF |
| | | | | | | | Human Patient | | VSSNYMSWVRQAPGKGLEWVSVIYSG |
| | | | | | | | | | GSTYYADSVKGRFTISRHNSKNTLYLQM |
| | | | | | | | | | NSLRAEDTAVYYCAREAYGMDVWGQG |
| | | | | | | | | | TTVTVSS |
| BD-236 | Ab | SARS-CoV2 | | SARS-CoV2 | | S; RBD | B-cells; SARS-CoV2 | 125 | EVQLVESGGGLIQPGGSLRLSCAASGITV |
| | | | | | | | Human Patient | | SSNYMSWVRQAPGKGLEWVSVIYSGG |
| | | | | | | | | | STDYADSVKGRFTISRDKSKNTLYLQMN |
| | | | | | | | | | SLRAEDTAVYYCARDLGEAGGMDVWG |
| | | | | | | | | | QGTTVTVSS |
| BD-368-2 | Ab | SARS-CoV2 | | SARS-CoV2 | | S; RBD | B-cells; SARS-CoV2 | | ND |
| | | | | | | | Human Patient | | |
| BD-494 | Ab | SARS-CoV2 | | SARS-CoV2 | | S; | Phage Display | | ND |
| | | | | | | probably | Library (Antibody, | | |
| | | | | | | RBD | human, immune - | | |
| | | | | | | (implied | CoV2) | | |
| | | | | | | by | | | |
| | | | | | | clustering) | | | |
| BD-498 | Ab | SARS-CoV2 | | SARS-CoV2 | | S; | Phage Display | | ND |
| | | | | | | probably | Library (Antibody, | | |
| | | | | | | RBD | human, immune - | | |
| | | | | | | (implied | CoV2) | | |
| | | | | | | by | | | |
| | | | | | | clustering) | | | |
| BD-500 | Ab | SARS-CoV2 | | SARS-CoV2 | | S; | B-cells; SARS-CoV2 | | ND |
| | | | | | | probably | Human Patient | | |
| | | | | | | RBD | | | |
| | | | | | | (implied | | | |
| | | | | | | by | | | |
| | | | | | | clustering) | | | |
| BD-503 | Ab | SARS-CoV2 | | SARS-CoV2 | | S; RBD | B-cells; SARS-CoV2 | | ND |
| | | | | | | | Human Patient | | |
| BD-504 | Ab | SARS-CoV2 | | SARS-CoV2 | | S; RBD | B-cells; SARS-CoV2 | | ND |
| | | | | | | | Human Patient | | |
| BD-505 | Ab | SARS-CoV2 | | SARS-CoV2 | | S; RBD | B-cells; SARS-CoV2 | | ND |
| | | | | | | | Human Patient | | |
| BD-506 | Ab | SARS-CoV2 | | SARS-CoV2 | | S; RBD | B-cells; SARS-CoV2 | | ND |
| | | | | | | | Human Patient | | |
| BD-507 | Ab | SARS-CoV2 | | SARS-CoV2 | | S; RBD | B-cells; SARS-CoV2 | | ND |
| | | | | | | | Human Patient | | |
| BD-508 | Ab | SARS-CoV2 | | SARS-CoV2 | | S; RBD | B-cells; SARS-CoV2 | | ND |
| | | | | | | | Human Patient | | |
| BD-515 | Ab | SARS-CoV2 | | SARS-CoV2 | | S; RBD | B-cells; SARS-CoV2 | | ND |
| | | | | | | | Human Patient | | |
| BD-604 | Ab | SARS-CoV2 | | SARS-CoV2 | | S; RBD | B-cells; SARS-CoV2 | 126 | EVQLVESGGGLIQPGGSLRLSCAASGIIV |
| | | | | | | | Human Patient | | SSNYMTWVRQAPGKGLEWVSVIYSGG |
| | | | | | | | | | STFYADSVKGRFTISRDNSKNTLYLQMSS |
| | | | | | | | | | LRAEDTAVYYCARDLGPYGMDVWGQG |
| | | | | | | | | | TTVTVSS |
| BD-629 | Ab | SARS-CoV2 | | SARS-CoV2 | | S; RBD | B-cells; SARS-CoV2 | 127 | EVQLVESGGGLIQPGGSLRLSCAASEFIV |
| | | | | | | | Human Patient | | SRNYMSWVRQAPGKGLEWVSVIYSGG |
| | | | | | | | | | STYYADSVKGRFTISRDNSKNTLNLQMN |
| | | | | | | | | | SLRAEDTAVYYCARDYGDYYFDYWGQG |
| | | | | | | | | | TLVTVSS |
| BD23 | Ab | SARS-CoV2 | | SARS-CoV2 | | S; RBD | B-cells; SARS-CoV2 | 128 | QVQLVQSGSELKKPGASVKVSCKASGYT |
| | | | | | | | Human Patient | | FTSYAMNWVRQAPGQGLEWMGWINT |
| | | | | | | | | | NTGNPTYAQGFTGRFVFSLDTSVSTAYL |
| | | | | | | | | | QISSLKAEDTAVYYCARPQGGSSWYRDY |
| | | | | | | | | | YYGMDVWGQGTTVTVSS |
| Bertoglio | Ab | SARS-CoV2 | | SARS-CoV2 | | S; RBD | Phage Display | | 17 |
| et al., | | | | | | | Library (Antibody, | | |
| 2020 | | | | | | | human, non- | | |
| | | | | | | | immune) | | |
| C002 | Ab | SARS-CoV2 | SARS-CoV1 | SARS-CoV2 | SARS-CoV1 | S; RBD | B-cells; SARS-CoV2 | 129 | EVQLVESGGGVVQPGRSLRLSCAASGFT |
| | | | | | | | Human Patient | | FSIYGMHWVRQAPGKGLEWVAVISYD |
| | | | | | | | | | GSNKYYADSVKGRFTISRDNSKNTLYLQ |
| | | | | | | | | | MNSLRAEDTAVYYCAKEGRPSDIVVVVA |
| | | | | | | | | | FDYWGQGTLVTVSS |
| C003 | Ab | SARS-CoV2 | SARS-CoV1 | SARS-CoV2 | SARS-CoV1 | S; RBD | B-cells; SARS-CoV2 | 130 | EVQLVESGGGLIQPGGSLRLSCAASGFT |
| | | | | (weak) | | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAGDTAVYYCARDYGDFYFDYWGQ |
| | | | | | | | | | GTLVTVSS |
| C004 | Ab | SARS-CoV2 | SARS-CoV1 | SARS-CoV2 | SARS-CoV1 | S; RBD | B-cells; SARS-CoV2 | 131 | QVQLVQSGAEVKKPGASVKVSCKASGY |
| | | | | | | | Human Patient | | TFTGYYMHWVRQAPGQGLEWMGWI |
| | | | | | | | | | NPISGGTNYAQKFQGRVTMTRDTSISTA |
| | | | | | | | | | YMELSRLRSDDTAVYYCASPASRGYSGY |
| | | | | | | | | | DHGYYYYMDVWGKGTTVTVSS |
| C005 | Ab | SARS-CoV2 | SARS-CoV1 | SARS-CoV2 | SARS-CoV1 | S; RBD | B-cells; SARS-CoV2 | 132 | QVQLVQSGPEVKKPGTSVKVSCKASGFT |
| | | | | | | | Human Patient | | FTSSAVQWVRQARGQRLEWIGWIVVG |
| | | | | | | | | | SGNTNYAQKFQERVTITRDMSTSTAYM |
| | | | | | | | | | ELSSLRSEDTAVYYCAAPHCSGGSCLDAF |
| | | | | | | | | | DIWGQGTMVTVSS |
| C006 | Ab | SARS-CoV2 | SARS-CoV1 | SARS-CoV2 | SARS-CoV1 | S; RBD | B-cells; SARS-CoV2 | 133 | QVQLVESGGGLVKPGGSLRLSCAASGFI |
| | | | | (weak) | | | Human Patient | | FSDYCMSWIRRAPGKGLEWLSYISNSGT |
| | | | | | | | | | TRYYADSVKGRFTISRDNGRNSLYLQMD |
| | | | | | | | | | SLSAEDTAVYYCARRGDGSSSIYYYNYM |
| | | | | | | | | | DVWGKGTTVTVSS |
| C008 | Ab | SARS-CoV2 | SARS-CoV1 | SARS-CoV2 | SARS-CoV1 | S; RBD | B-cells; SARS-CoV2 | 134 | EVQLVESGGGVVQPGRSLRLSCAASGFT |
| | | | | (weak) | | | Human Patient | | FSSYGMHWVRQAPGKGLEWVTVISYD |
| | | | | | | | | | GRNKYYADSVKGRFTISRDNSKNTLYLQ |
| | | | | | | | | | MNSLRAEDTAVYYCAREFGDPEWYFDY |
| | | | | | | | | | WGQGTLVTVSS |
| C009 | Ab | SARS-CoV2 | SARS-CoV1 | SARS-CoV2 | SARS-CoV1 | S; RBD | B-cells; SARS-CoV2 | 135 | QVQLVQSGAEVKKPGASVKVSCMASGY |
| | | | | | | | Human Patient | | TFTGYYMHWVRQAPGQGLEWMGWI |
| | | | | | | | | | NPNSGGTNYAQKFQGRVTMTRDTSIST |
| | | | | | | | | | AYMELSRLRSDDTAVYYCARDSPFSALG |
| | | | | | | | | | ASNDYWGQGTLVTVSS |
| C010 | Ab | SARS-CoV2 | SARS-CoV1 | | SARS-CoV1, | S; RBD | B-cells; SARS-CoV2 | 136 | EVQLVESGGGVVQPGRSLRLSCAASGFT |
| | | | | | SARS-CoV2 | | Human Patient | | FSSYAMHWVRQAPAKGLEWVAVILYD |
| | | | | | | | | | GSGKYYADSVKGRFTISRDNSKNTLYLQ |
| | | | | | | | | | MNSLRAEDTAVYYCARDGIVDTALVTW |
| | | | | | | | | | FDYWGQGTLVTVSS |
| C013 | Ab | SARS-CoV2 | SARS-CoV1 | SARS-CoV2 | SARS-CoV1 | S; RBD | B-cells; SARS-CoV2 | 137 | QVQLVQSGAEVKKPGSSVKVSCKASGG |
| | | | | (weak) | | | Human Patient | | TFSSYAISWVRQAPGQGLEWMGGIIPIF |
| | | | | | | | | | GTANYAQKFQGRVTITADESTSTAYMEL |
| | | | | | | | | | SSLRSEDTAVYYCARGNRLLYCSSTSCYL |
| | | | | | | | | | DAVRQGYYYYYYMDVWGKGTTVTVSS |
| C016 | Ab | SARS-CoV2 | SARS-CoV1 | | SARS-CoV1, | S; RBD | B-cells; SARS-CoV2 | 138 | EVQLVESGGGVVQPGRSLRLSCAASGFT |
| | | | | | SARS-CoV2 | | Human Patient | | FSRYGMHWVRQAPGKGLEWVAVISYD |
| | | | | | | | | | GSNKYYADSVKGRFTISRDNSKNTLYLQ |
| | | | | | | | | | MNSLRAEDTAVYYCAKVTAPYCSGGSCY |
| | | | | | | | | | GGNFDYWGQGTLVTVSS |
| C017 | Ab | SARS-CoV2 | SARS-CoV1 | SARS-CoV2 | SARS-CoV1 | S; RBD | B-cells; SARS-CoV2 | 139 | EVQLVESGGGLVQPGRSLRLSCAASGFT |
| | | | | | | | Human Patient | | FDDYAMHWVRQAPGKGLEWVSGISW |
| | | | | | | | | | NSGTIGYADSVKGRFTISRDNAKNSLYLQ |
| | | | | | | | | | MNSLRAEDTAFYYCAKAGVRGIAAAGP |
| | | | | | | | | | DLNFDHWGQGTLVTVSS |
| C018 | Ab | SARS-CoV2 | SARS-CoV1 | | SARS-CoV1, | S; RBD | B-cells; SARS-CoV2 | 140 | EVQLVESGGGVVQPGRSLRLSCAASGFT |
| | | | | | SARS-CoV2 | | Human Patient | | FSNYAIHWVRQAPGKGLEWVAVISYDG |
| | | | | | | | | | SNKYYADSVKGRFTISRDNSKNTLYLQM |
| | | | | | | | | | NSLRAEDTAVYYCARDFDDSSFWAFDY |
| | | | | | | | | | WGQGTLVTVSS |
| C019 | Ab | SARS-CoV2 | SARS-CoV1 | | SARS-CoV1, | S; RBD | B-cells; SARS-CoV2 | 141 | QVQLVQSGAEVKKPGASVKVSCKASGY |
| | | | | | SARS-CoV2 | | Human Patient | | TFTSYYMHWVRQAPGQGLEWMGIINP |
| | | | | | | | | | SGGSTSYAQKFQGRVTMTRDTSTSTVY |
| | | | | | | | | | MELSSLRSEDTAVYYCARVPREGTPGFD |
| | | | | | | | | | PWGQGTLVTVSS |
| C021 | Ab | SARS-CoV2 | SARS-CoV1 | | SARS-CoV1, | S; RBD | B-cells; SARS-CoV2 | 142 | QVQLQESGPGLVKPSQTLSLTCTVSGGSI |
| | | | | | SARS-CoV2 | | Human Patient | | SSGGYYWSWIRQHPGKGLEWIGYIYYS |
| | | | | | | | | | GSTYYNPSLKSRVTISVDTSKNQFSLKLS |
| | | | | | | | | | SVTAADTAVYYCARVWQYYDSSGSFDYW |
| | | | | | | | | | GQGTLVTVSS |
| C022 | Ab | SARS-CoV1, | | SARS-CoV2 | | S; RBD | B-cells; SARS-CoV2 | 143 | QVQLQESGPGLVKPSETLSVTCTVSGGSI |
| | | SARS-CoV2 | | | | | Human Patient | | SSSRYYWGWIRQPPGKGLEWIGSIYYSG |
| | | | | | | | | | STYYNPSLKSRVTISVDTSKNQFSLKLSS |
| | | | | | | | | | VTAADTAVYYCARHAAAYYDRSGYYFIEY |
| | | | | | | | | | FQHWGQGTLVTVSS |
| C027 | Ab | SARS-CoV1 | | | SARS-CoV2 | S; RBD | B-cells; SARS-CoV2 | 144 | EVQLVESGGGVVQPGRSLRLSCAASGFT |
| | | (weak), | | | | | Human Patient | | FSSYGMHWVRQAPGKGLEWVAVISYD |
| | | SARS-CoV2 | | | | | | | GSNKYYADSVKGRFTISRDNSKNTLYLQ |
| | | | | | | | | | MNSLRAEDTAVYYCAKASGIYCSGGDCY |
| | | | | | | | | | SYYFDYWGQGTLVTVSS |
| C029 | Ab | SARS-CoV2 | SARS-CoV1 | | SARS-CoV1, | S; RBD | B-cells; SARS-CoV2 | 145 | QVQLQESGPGLVKPSQTLSLTCTVSGGSI |
| | | | | | SARS-CoV2 | | Human Patient | | SSGGYYWSWIRQHPGKGLEWIGYIYYS |
| | | | | | | | | | GSTYYNPSLKSRVTISVDTSKNQFSLKLS |
| | | | | | | | | | SVTAADTAVYYCARTMYYYDSSGSFDYW |
| | | | | | | | | | GQGTLVTVSS |
| C030 | Ab | SARS-CoV2 | SARS-CoV1 | | SARS-CoV1, | S; RBD | B-cells; SARS-CoV2 | 146 | EVQLVESGGGVVQPGRSLRLSCAASGFT |
| | | | | | SARS-CoV2 | | Human Patient | | FSSYGMHWVRQAPGKGLEWVAVISYD |
| | | | | | | | | | GSNKYYADSVKGRFTISRDNSKNTLYLQ |
| | | | | | | | | | MNSLRAEDTAVYYCAKASGIYCSGGNCY |
| | | | | | | | | | SYYFDYWGQGTLVTVSS |
| C031 | Ab | SARS-CoV2 | SARS-CoV1 | | SARS-CoV1, | S; RBD | B-cells; SARS-CoV2 | 147 | EVQLVESGGGLVQPGGSLRLSCAASGFT |
| | | | | | SARS-CoV2 | | Human Patient | | FSSYDMHWVRQATGKGLEWVSAIGTA |
| | | | | | | | | | GDTYYPGSVKGRFTISRENAKNSLYLQM |
| | | | | | | | | | NSLRAGDTAVYYCARVGYDSSGYSGWY |
| | | | | | | | | | FDLWGRGTLVTVSS |
| C101 | Ab | SARS-CoV2 | SARS-CoV1 | SARS-CoV2 | SARS-CoV1 | S; RBD | B-cells; SARS-CoV2 | 148 | QVQLVESGGGLIQPGGSLRLSCAASGFI |
| | | | | | | | Human Patient | | VSSNYMSWVRQAPGKGLEWVSVIYSG |
| | | | | | | | | | GSTFYTDSVKGRFTISRDNSKNTLYLQM |
| | | | | | | | | | NSLRAEDTAVYYCVRDYGDFYFDYWGQ |
| | | | | | | | | | GTLVTVSS |
| C102 | Ab | SARS-CoV2 | SARS-CoV1 | SARS-CoV2 | SARS-CoV1 | S; RBD | B-cells; SARS-CoV2 | 149 | QVQLVESGGGLIQPGGSLRLSCAASGFI |
| | | | | | | | Human Patient | | VSSNYMSWVRQAPGKGLEWVSVIYSG |
| | | | | | | | | | GSTFYADSVKGRFTISRDNSKNTLYLQM |
| | | | | | | | | | NSLRAEDTAVYYCARDYGDYYFDYWGQ |
| | | | | | | | | | GTLVTVSS |
| C103 | Ab | SARS-CoV2 | SARS-CoV1 | SARS-CoV2 | SARS-CoV1 | S; RBD | B-cells; SARS-CoV2 | 150 | QVQLQQWGAGLLKPSETLSLTCAVSGG |
| | | | | | | | Human Patient | | SLSGFYWTWIRQPPGKGLEWIGETNHF |
| | | | | | | | | | GSTGYKPSLKSRVTISVDMSRNQFSLKVT |
| | | | | | | | | | SVTAADTAVYYCARKPLLYSDFSPGAFDI |
| | | | | | | | | | WGQGTMVTVSS |
| C104 | Ab | SARS-CoV2 | SARS-CoV1 | SARS-CoV2 | SARS-CoV1 | S; RBD | B-cells; SARS-CoV2 | 151 | QVQLQQWGAGLLKPSETLSLSCAVYGG |
| | | | | | | | Human Patient | | SLSGYYWSWIRQPPGKGLEWIGEINHF |
| | | | | | | | | | GSTGYNPSLKSRVTISVDTSKSQFSVKLS |
| | | | | | | | | | SVTAADTAVYYCARKPLLYSNLSPGAFDI |
| | | | | | | | | | WGQGTMVTVSS |
| C105 | Ab | SARS-CoV2 | SARS-CoV1 | SARS-CoV2 | SARS-CoV1 | S; RBD | B-cells; SARS-CoV2 | 152 | QVQLVESGGGLIQPGGSLRLSCAASGFT |
| | | | | | | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAEDTAVYYCARGEGWELPYDYWG |
| | | | | | | | | | QGTLVTVSS |
| C106 | Ab | SARS-CoV1, | | | SARS-CoV2 | S; RBD | B-cells; SARS-CoV2 | 153 | QLQLQESGPGLVKPSETLSLTCTVSGASV |
| | | SARS-CoV2 | | | | | Human Patient | | SSGSYYWSWIRQPPGKGLEWIGYIYYSG |
| | | | | | | | | | STNYNPSLKSRVTISVDTSKNQFSLKLSS |
| | | | | | | | | | VTAADTAVYYCARERPGGTYSNTWYTPT |
| | | | | | | | | | DTNWFDTWGQGTLVTVSS |
| C107 | Ab | SARS-CoV2 | SARS-CoV1 | | SARS-CoV1, | S; RBD | B-cells; SARS-CoV2 | 154 | QVQLVQSGAEVKKPGASVRVSCKASGY |
| | | | | | SARS-CoV2 | | Human Patient | | TFTSYGFSWVRQAPGQGLEWMGWISA |
| | | | | | | | | | YNGNTNFAQKLQGRVTMTTDTSTSTAY |
| | | | | | | | | | MELRSLRSDDTAVYYCARGEAVAGTTGF |
| | | | | | | | | | FDYWGQGTLVTVSS |
| C108 | Ab | SARS-CoV2 | SARS-CoV1 | SARS-CoV2 | SARS-CoV1 | S; RBD | B-cells; SARS-CoV2 | 155 | QVQLQESGPGLVKPSGTLSLTCAVSGGS |
| | | | | (weak) | | | Human Patient | | ISSTNWWSWVRQPPGKGLEWIGEIYHT |
| | | | | | | | | | GSTNYNPSLKSRVTISVDKSKNQFSLKLS |
| | | | | | | | | | SVTAADTAVYYCVRDGGRPGDAFDIWG |
| | | | | | | | | | QGTMVTVSS |
| C109 | Ab | SARS-CoV2 | | | | S; RBD | B-cells; SARS-CoV2 | 156 | EVQLVESGGGLVQPGGSLRLSCAASGFT |
| | | | | | | | Human Patient | | FSSYWMSWVRQAPGKGLEWVANIKQ |
| | | | | | | | | | DGSEKYYVDSVKGRFTISGDNAKNSLYL |
| | | | | | | | | | HMNSLRAEDTAVYYCAIQLWLRGGYDY |
| | | | | | | | | | WGQGTLVTVSS |
| C110 | Ab | SARS-CoV2 | SARS-CoV1 | SARS-CoV2 | SARS-CoV1 | S; RBD | B-cells; SARS-CoV2 | 157 | QVQLQQSGAEVKKPGESLKISCKGSGYS |
| | | | | | | | Human Patient | | FTSYWIGWVRQMPGKGLEWMGIIYPG |
| | | | | | | | | | DSDTRYSPSFQGQVTISADKSISTAYMQ |
| | | | | | | | | | WSSLKASDTAMYYCARSFRDDPRIAVA |
| | | | | | | | | | GPADAFDIWGQGTMVTVSS |
| C111 | Ab | SARS-CoV2 | | | | S; RBD | B-cells; SARS-CoV2 | 158 | QLQLQESGPGLVKPSETLSLTCTVSGGSI |
| | | | | | | | Human Patient | | SSYYWSWIRQPPGKGLEWIGYIYYSGST |
| | | | | | | | | | NYNPSLKSRVTISVDTSKNQFSLKLSSVT |
| | | | | | | | | | AADTAVYYCARVEDWGYCSSTNCYSGAF |
| | | | | | | | | | DIWGQGTMVTVSS |
| C112 | Ab | SARS-CoV2 | SARS-CoV1 | SARS-CoV2 | SARS-CoV1 | S; RBD | B-cells; SARS-CoV2 | 159 | QVQLVESGGGVVQPGRSLRLSCAASGF |
| | | | | (weak) | | | Human Patient | | TFSSHAMHWVRQAPGKGLEWVAVISY |
| | | | | | | | | | DGSNKYYADSVKGRFTISRDNSKNTLYL |
| | | | | | | | | | QMNSLRAEDTAVYYCAREDYYDSSGSF |
| | | | | | | | | | DYWGQGTLVTVSS |
| C113 | Ab | SARS-CoV2 | SARS-CoV1 | | SARS-CoV1, | S; RBD | B-cells; SARS-CoV2 | 160 | QVQLVESGGGVVQPGRSLRLSCAASGF |
| | | | | | SARS-CoV2 | | Human Patient | | TFSNFGMHWVRQAPGKGLEWVAVIW |
| | | | | | | | | | YDGSNKYYADSVKGRFTISRDNSKNTLYL |
| | | | | | | | | | QMNSLRAEDTAVYYCARGVNPDDILTG |
| | | | | | | | | | VDAFDIWGQGTMVTVSS |
| C114 | Ab | SARS-CoV2 | SARS-CoV1 | | SARS-CoV1, | S; RBD | B-cells; SARS-CoV2 | 161 | QVQLVESGGGLIQPGGSLKLSCVVSGFT |
| | | | | | SARS-CoV2 | | Human Patient | | VSKNYISWVRQAPGKGLEWVSVIFAGG |
| | | | | | | | | | STFYADSVKGRFAISRDNSNNTLFLQMN |
| | | | | | | | | | SLRVEDTAIYYCARGDGELFFDQWGQG |
| | | | | | | | | | TLVTVSS |
| C115 | Ab | SARS-CoV2 | SARS-CoV1 | SARS-CoV2 | SARS-CoV1 | S; RBD | B-cells; SARS-CoV2 | 162 | QVQLVESGGGLIKPGRSLRLSCTASGFTF |
| | | | | (weak) | | | Human Patient | | GDYAMTWFRQAPGKGLEWVGFIRSKA |
| | | | | | | | | | YGGTTGYAASVKYRFTISRDDSKSIAYLQ |
| | | | | | | | | | MDSLKTEDTAVYYCTRWDGWSQHDY |
| | | | | | | | | | WGQGTLVTVSS |
| C116 | Ab | SARS-CoV2 | SARS-CoV1 | | SARS-CoV1, | S; RBD | B-cells; SARS-CoV2 | 163 | QVQLVESGGGVVQPGRSLRLSCAASGF |
| | | | | | SARS-CoV2 | | Human Patient | | TYSTYAMHWVRQAPGKGLEWVAFISYD |
| | | | | | | | | | GSNKYYADSVKGRFTISRDNSKNTLYLQ |
| | | | | | | | | | MNSLRAEDTAVYYCARDFYHNWFDPW |
| | | | | | | | | | GQGTLVTVSS |
| C117 | Ab | SARS-CoV2 | SARS-CoV1 | SARS-CoV2 | SARS-CoV1 | S; RBD | B-cells; SARS-CoV2 | 164 | QVQLVESGGGVVQPGRSLRLSCAASGF |
| | | | | (weak) | | | Human Patient | | TFSTYAMHWVRQAPGEGLEWVAVISY |
| | | | | | | | | | DGSNTYYADSVKGRFTISRDNSKNTLYL |
| | | | | | | | | | QMNSLRAEDTAVYYCARDPIWFGELLSP |
| | | | | | | | | | PFVHFDYWGQGTLVTVSS |
| C118 | Ab | SARS-CoV1, | | SARS-CoV2 | | S; RBD | B-cells; SARS-CoV2 | 165 | QVQLVESGGGVVQPGRSLRLSCAASGF |
| | | SARS-CoV2 | | (weak) | | | Human Patient | | TFSNYAMHWVRQAPGKGLEWVAVISY |
| | | | | | | | | | DGSNKYYADSVKGRFTISRDNSKNTLYL |
| | | | | | | | | | QMNSLRAEDTAIYYCASGYTGYDYFVRG |
| | | | | | | | | | DYYGLDVWGQGTTVTVSS |
| C119 | Ab | SARS-CoV2 | SARS-CoV1 | SARS-CoV2 | SARS-CoV1 | S; RBD | B-cells; SARS-CoV2 | 166 | QVQLVQSGAEVKKPGASVKVSCKASGY |
| | | | | | | | Human Patient | | TFTSYYMHWVRQAPGQGLEWMGIINP |
| | | | | | | | | | SGGSTSYAQKLQGRVTMTRDTSTSTVY |
| | | | | | | | | | MELSSLRSEDTAVYYCARANHETTMDTY |
| | | | | | | | | | YYYYYMDVWGKGTTVTVSS |
| C120 | Ab | SARS-CoV2 | SARS-CoV1 | SARS-CoV2 | SARS-CoV1 | S; RBD | B-cells; SARS-CoV2 | 167 | EVQLVESGGGLIQPGGSLRLSCAASGFT |
| | | | | | | | Human Patient | | VSSNYMTWVRQAPGKGLEWVSLIYPG |
| | | | | | | | | | GSTYYADSVKGRFTISRDNSKNTLYLQM |
| | | | | | | | | | NSLRAEDTAVYYCAREGMGMAAAGTW |
| | | | | | | | | | GQGTLVTVSS |
| C121 | Ab | SARS-CoV2 | SARS-CoV1 | SARS-CoV2 | SARS-CoV1 | S; RBD | B-cells; SARS-CoV2 | 168 | QVQLVQSGAEVKKPGASVKVSCKASGY |
| | | | | | | | Human Patient | | TFTGYYMHWVRQAPGQGLEWMGWIS |
| | | | | | | | | | PVSGGTNYAQKFQGRVTMTRDTSISTAY |
| | | | | | | | | | MELSRLRSDDTAVYYCARAPLFPTGVLA |
| | | | | | | | | | GDYYYYGMDVWGQGTTVTVSS |
| C122 | Ab | SARS-CoV2 | SARS-CoV1 | SARS-CoV2 | SARS-CoV1 | S; RBD | B-cells; SARS-CoV2 | 169 | EVQLVESGGGLIQPGGSLRLSCAASGLT |
| | | | | | | | Human Patient | | VSSNYMSWVRQAPGKGLEWVSVLYSG |
| | | | | | | | | | GSSFYADSVKGRFTISRDNSKNTLYLQM |
| | | | | | | | | | NSLRAEDTAVYYCARESGDTTMAFDYW |
| | | | | | | | | | GQGTLVTVSS |
| C123 | Ab | SARS-CoV2 | SARS-CoV1 | SARS-CoV2 | SARS-CoV1 | S; RBD | B-cells; SARS-CoV2 | 170 | EVQLVESGGGLIQPGGSLRLSCAASGVT |
| | | | | | | | Human Patient | | VSRNYMSWVRQAPGKGLEWVSVIYSG |
| | | | | | | | | | GSTYYADSVKGRFTISRDNSKNTLYLQM |
| | | | | | | | | | NSLRAEDTAVYYCARDLSAAFDIWGQG |
| | | | | | | | | | TMVTVSS |
| C124 | Ab | SARS-CoV2 | SARS-CoV1 | SARS-CoV2 | SARS-CoV1 | S; RBD | B-cells; SARS-CoV2 | 171 | EVQLVESGGGLVQPGGSLRLSCAASGFT |
| | | | | (weak) | | | Human Patient | | FSGYSMNWVRQAPGKGPEWVSYISRSS |
| | | | | | | | | | STIYYADSVKGRFTISRDNAKNSLYLQMN |
| | | | | | | | | | SLRDEDTAVYYCAREGARVGATYDTYYF |
| | | | | | | | | | DYWGQGTLVTVSS |
| C125 | Ab | SARS-CoV2 | SARS-CoV1 | SARS-CoV2 | SARS-CoV1 | S; RBD | B-cells; SARS-CoV2 | 172 | QVQLVQSGPEVKKPGTSVKVSCKASGFT |
| | | | | | | | Human Patient | | FTSSAVQWVRQARGQRLEWIGWIVVG |
| | | | | | | | | | SGNTNYAQKFQERVTITRDMSTSTAYM |
| | | | | | | | | | ELSSLRSEDTAVYYCAAPYCSGGSCSDAF |
| | | | | | | | | | DIWGQGTMVTVSS |
| C126 | Ab | SARS-CoV2 | SARS-CoV1 | | SARS-CoV1, | S; RBD | B-cells; SARS-CoV2 | 173 | QVQLQESGPGLVKPSETLSLSCAVSGGSI |
| | | | | | SARS-CoV2 | | Human Patient | | GSYFWSWIRQPPGKGLEWIGYLHYSGS |
| | | | | | | | | | TNYNPSLKSRVTISVDTSKNQFSLKLSSV |
| | | | | | | | | | TAADTAVYYCARLQWLRGAFDIWGQGT |
| | | | | | | | | | MVTVSS |
| C127 | Ab | SARS-CoV2 | SARS-CoV1 | SARS-CoV2 | SARS-CoV1 | S; RBD | B-cells; SARS-CoV2 | 174 | QVQLVQSGAEVKKPGASVKVSCKASGY |
| | | | | | | | Human Patient | | TFTGYYMHWVRQAPGQGLEWMGWI |
| | | | | | | | | | NPNSGGTNYAQKFQGRVTMTRDTSIST |
| | | | | | | | | | AYMELSRLRSDDTAVYYCATAHPRRIQG |
| | | | | | | | | | VFFLGPGVWGQGTTVTVSS |
| C128 | Ab | SARS-CoV2 | SARS-CoV1 | SARS-CoV2 | SARS-CoV1 | S; RBD | B-cells; SARS-CoV2 | 175 | EVQLLESGGGLVQPGGSLRLSCAASGFT |
| | | | | | | | Human Patient | | FSTYAMSWVRQAPGKGLEWVSTITGSG |
| | | | | | | | | | RDTYYADSVKGRFTISRDNSKNTLFLQLN |
| | | | | | | | | | SLRAEDAAVYSCANHPLASGDDYYHYY |
| | | | | | | | | | MDVWGKGTTVTVSS |
| C130 | Ab | SARS-CoV2 | SARS-CoV1 | | SARS-CoV1, | S; RBD | B-cells; SARS-CoV2 | 176 | QVQLVQSGAEVKKPGASVKVSCKASGY |
| | | | | | SARS-CoV2 | | Human Patient | | TFTNYYMHWVRQAPGQGLEWMGIINP |
| | | | | | | | | | SGGSTGYAQKFQGRVTMTRDTSTSTVY |
| | | | | | | | | | MELSSLRSEDTAVYYCARSRPTPDWYFD |
| | | | | | | | | | LWGRGTLVTVSS |
| C131 | Ab | SARS-CoV2 | SARS-CoV1 | SARS-CoV2 | SARS-CoV1 | S; RBD | B-cells; SARS-CoV2 | 177 | QVQLVQSGSEVKKPGSSVKVSCKASGG |
| | | | | | | | Human Patient | | TFSSYAFSWVRQAPGQGLEWMGRIIPIL |
| | | | | | | | | | ALANYAQKFQGRVTITADKSTSTAYMEL |
| | | | | | | | | | SSLRSEDTAVYYCARVNQAVTTPFSMDV |
| | | | | | | | | | WGQGTTVTVSS |
| C132 | Ab | SARS-CoV2 | SARS-CoV1 | SARS-CoV2 | SARS-CoV1 | S; RBD | B-cells; SARS-CoV2 | 178 | QVQLQESGPGLVKPSGTLSLTCAVSGGS |
| | | | | (weak) | | | Human Patient | | ISSNNWWSCVRQPPGKGLEWIGEIYHS |
| | | | | | | | | | GSTNYNPSLKSRVTISVDKSKNQFSLKLS |
| | | | | | | | | | SVTAADTAVYYCARGGDTAMGPEYFDY |
| | | | | | | | | | WGQGTLVTVSS |
| C133 | Ab | SARS-CoV2 | SARS-CoV1 | | SARS-CoV1, | S; RBD | B-cells; SARS-CoV2 | 179 | QVQLVESGGGVVQPGRSLRLSCAASGF |
| | | | | | SARS-CoV2 | | Human Patient | | TFSSYAMHWVRQAPGKGLEWVAVILY |
| | | | | | | | | | DGSNKYYADSVKGRFTISRDNSKNTLYL |
| | | | | | | | | | QMNSLRAEDTAVYYCARDSDVDTSMVT |
| | | | | | | | | | WFDYWGQGTLVTVSS |
| C134 | Ab | SARS-CoV2 | SARS-CoV1 | | SARS-CoV1, | S; RBD | B-cells; SARS-CoV2 | 180 | EVQLLESGGGLVQPGGSLRLSCAASGFT |
| | | | | | SARS-CoV2 | | Human Patient | | FSNYAMSWVRQAPGKGLEWVSAISGS |
| | | | | | | | | | DGSTYYAGSVKGRFTISRDNSKNTLYLQ |
| | | | | | | | | | MNSLRAEDTAVYYCAKDPLITGPTYQYF |
| | | | | | | | | | HYWGQGTLVTVSS |
| C135 | Ab | SARS-CoV2 | SARS-CoV1 | SARS-CoV2 | SARS-CoV1 | S; RBD | B-cells; SARS-CoV2 | 181 | QVQLVESGGGVVQPGRSLRLSCAASGF |
| | | | | | | | Human Patient | | TFSSYAMHWVRQAPGKGLEWVAVIPF |
| | | | | | | | | | DGRNKYYADSVTGRFTISRDNSKNTLYL |
| | | | | | | | | | QMNSLRAEDTAVYYCASSSGYLFHSDY |
| | | | | | | | | | WGQGTLVTVSS |
| C138 | Ab | SARS-CoV2 | SARS-CoV1 | | SARS-CoV1, | S; RBD | B-cells; SARS-CoV2 | 182 | EVQLVESGGGLVQPGGSLRLSCAASGFT |
| | | | | | SARS-CoV2 | | Human Patient | | FSTYWMSWVRQPPGKGLEWVANIKQ |
| | | | | | | | | | DGSEKYYVDSVKGRFTISRDNAKNSLYL |
| | | | | | | | | | QMNSLRADDTAVYYCAGGTWLRSSFDY |
| | | | | | | | | | WGQGTLVTVSS |
| C139 | Ab | SARS-CoV2 | SARS-CoV1 | | SARS-CoV1, | S; RBD | B-cells; SARS-CoV2 | 183 | EVQLVESGGGVVQPGRSLRLSCAASGFT |
| | | | | | SARS-CoV2 | | Human Patient | | FSSYAMHWVRQAPGKGLEWVAVISYD |
| | | | | | | | | | GSNKYSADSVKGRFTISRDNSKNTLYLQ |
| | | | | | | | | | MNSLRAEDTAVYYCAKGGAYSYYYYMD |
| | | | | | | | | | VWGKGTTVTVSS |
| C140 | Ab | SARS-CoV2 | SARS-CoV1 | SARS-CoV2 | SARS-CoV1 | S; RBD | B-cells; SARS-CoV2 | 184 | EVQLVESGGGLVQPGGSLRLSCAASGVT |
| | | | | | | | Human Patient | | VSSNYMSWVRQAPGKGLEWVSLIYSGG |
| | | | | | | | | | STFYADSVKGRFTISRDNSENTLYLQMN |
| | | | | | | | | | TLRAEDTAVYYCARDLYYYGMDVWGQ |
| | | | | | | | | | GTTVTVSS |
| C141 | Ab | SARS-CoV2 | SARS-CoV1 | | SARS-CoV1, | S; RBD | B-cells; SARS-CoV2 | 185 | EVQLVESGGGVVQPGRSLRLSCAASGFT |
| | | | | | SARS-CoV2 | | Human Patient | | FSSYAMFWVRQAPGKGLEWVAVISYD |
| | | | | | | | | | GSNKYYADSVKGRFTISRDNSKNTLYLQ |
| | | | | | | | | | MNSLRAEDTAVYYCARADLGYCTNGVC |
| | | | | | | | | | YVDYWGQGTLVTVSS |
| C143 | Ab | SARS-CoV2 | SARS-CoV1 | | SARS-CoV1, | S; RBD | B-cells; SARS-CoV2 | 186 | EVQLVESGGGLVQPGGSLRLSCAASGFS |
| | | | | | SARS-CoV2 | | Human Patient | | VSTKYMTWVRQAPGKGLEWVSVLYSG |
| | | | | | | | | | GSDYYADSVKGRFTISRDNSKNALYLQM |
| | | | | | | | | | NSLRVEDTGVYYCARDSSEVRDHPGHP |
| | | | | | | | | | GRSVGAFDIWGQGTMVTVSS |
| C144 | Ab | SARS-CoV2 | SARS-CoV1 | SARS-CoV2 | SARS-CoV1 | S; RBD | B-cells; SARS-CoV2 | 187 | EVQLVESGGGLIQPGGSLRLSCAASGFT |
| | | | | | | | Human Patient | | VSNNYMSWVRQAPGKGLEWVSVIYSG |
| | | | | | | | | | GSTYYADSVKGRFTISRDKSKNTLYLQM |
| | | | | | | | | | NRLRAEDTAVYYCAREGEVEGYNDFWS |
| | | | | | | | | | GYSRDRYYFDYWGQGTLVTVSS |
| C145 | Ab | SARS-CoV2 | SARS-CoV1 | SARS-CoV2 | SARS-CoV1 | S; RBD | B-cells; SARS-CoV2 | 188 | EVQLVESGGGLIQPGGSLRLSCAASGFS |
| | | | | | | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAEDTAVYYCAREGEVEGYYDFWS |
| | | | | | | | | | GYSRDRYYFDYWGQGTLVTVSS |
| C146 | Ab | SARS-CoV2 | SARS-CoV1 | | SARS-CoV1, | S; non- | B-cells; SARS-CoV2 | 189 | EVQLVESGGGLVKPGGSLRLSCAASGLT |
| | | | | | SARS-CoV2 | RBD | Human Patient | | FTAYRMNWVRQAPGKGLEWLSSISNTN |
| | | | | | | | | | GDIYYADSVKGRFTISRDNAKNSLYLQM |
| | | | | | | | | | NSLRADDTAVYYCARDVASNYAYFDLW |
| | | | | | | | | | GQGTLVTVSS |
| C147 | Ab | SARS-CoV2 | SARS-CoV1 | | SARS-CoV1, | S; non- | B-cells; SARS-CoV2 | 190 | EVQLVQSGAEVKKPGESLKISCKGSGYR |
| | | | | | SARS-CoV2 | RBD | Human Patient | | FTNYWIGWVRQMPGKGLEWMGIIYPG |
| | | | | | | | | | DSDTRYSPSFQGQVTISADKSITTAYLQW |
| | | | | | | | | | SSLKASDTAMYYCARLSDRWYSPFDPW |
| | | | | | | | | | GQGTLVTVSS |
| C148 | Ab | SARS-CoV2 | SARS-CoV1 | | SARS-CoV1, | S; RBD | B-cells; SARS-CoV2 | 191 | EVQLVESGGGLVQPGGSQRLSCAASGF |
| | | | | | SARS-CoV2 | | Human Patient | | TVSSNYMSWIRQAPGKGLEWVSVIYSG |
| | | | | | | | | | GSAYYVDSVKGRFTISRDNSKNTLYLQM |
| | | | | | | | | | NSLRPEDTAVYYCARIANYMDVWGKGT |
| | | | | | | | | | TVTVSS |
| C150 | Ab | SARS-CoV2 | SARS-CoV1 | | SARS-CoV1, | S; RBD | B-cells; SARS-CoV2 | 192 | EVQLVESGGGLVQPGGSLRLSCVASGFT |
| | | | | | SARS-CoV2 | | Human Patient | | FSSYWMHWVRQVPGKGPVWVSHINS |
| | | | | | | | | | EGSSTNYADSVRGRFTISRDNAKDTLYL |
| | | | | | | | | | QMNNLRAEDTAVYYCARPTAVAAAGN |
| | | | | | | | | | YFYYYGMDVWGQGTTVTVSS |
| C151 | Ab | SARS-CoV2 | SARS-CoV1 | SARS-CoV2 | SARS-CoV1 | S; RBD | B-cells; SARS-CoV2 | 193 | EVQLVESGGGLVKPGGSLRLSCAASGFT |
| | | | | (weak) | | | Human Patient | | FSSYNMNWVRQAPGKGLEWVSCISSSS |
| | | | | | | | | | SYIYYADSVKGRFTISRDNAKNSLYLQMN |
| | | | | | | | | | SLRAEDTAVYYCARERGYDGGKTPPFLG |
| | | | | | | | | | GQGTLVTVSS |
| C153 | Ab | SARS-CoV2 | SARS-CoV1 | SARS-CoV2 | SARS-CoV1 | S; RBD | B-cells; SARS-CoV2 | 194 | EVQLVESGGGLIQPGGSLRLSCAASGFT |
| | | | | (weak) | | | Human Patient | | VSSNYMSWVRQAPGKGLEWVSVIYSGY |
| | | | | | | | | | STYYVDSVKGRFTISRDNSKNTLYLQMN |
| | | | | | | | | | SLRAEDTAVYYCARVGGAHSGYDGSFDY |
| | | | | | | | | | WGQGTLVTVSS |
| C154 | Ab | SARS-CoV1, | | SARS-CoV2 | | S; RBD | B-cells; SARS-CoV2 | 195 | QVQLVESGGGVVQPGRSLRLSCAASGF |
| | | SARS-CoV2 | | (weak) | | | Human Patient | | TFSRYGMHWVRQAPGKGLEWVAVMS |
| | | | | | | | | | YDGSSKYYADSVKGRFTISRDNSKNTLCL |
| | | | | | | | | | QMNSLRAEDTAVYYCAKQAGPYCSGGS |
| | | | | | | | | | CYSAPFDYWGQGTLVTVSS |
| C155 | Ab | SARS-CoV2 | SARS-CoV1 | SARS-CoV2 | SARS-CoV1 | S; RBD | B-cells; SARS-CoV2 | 196 | EVQLVESGGGLIQPGGSLRLSCAASGFIV |
| | | | | | | | Human Patient | | SSNYMSWVRQAPGKGLEWVSVIYSGG |
| | | | | | | | | | STFYADSVKGRFTISRDNSKNTLYLQMN |
| | | | | | | | | | SLRAEDTAVYYCARDFGEFYFDYWGQG |
| | | | | | | | | | TLVTVSS |
| C156 | Ab | SARS-CoV2 | SARS-CoV1 | | SARS-CoV1, | S; RBD | B-cells; SARS-CoV2 | 197 | QVQLVESGGGVVQPGRSLRLSCAASGF |
| | | | | | SARS-CoV2 | | Human Patient | | TFSNYGMHWVRQAPGKGLEWVAVISY |
| | | | | | | | | | DGNNKYYADSVKGRFTISRDNSKNTLYL |
| | | | | | | | | | QMNSLRAEDTAVYYCAKDPFPLAVAGT |
| | | | | | | | | | GYFDYWGQGTLVTVSS |
| C164 | Ab | SARS-CoV2 | SARS-CoV1 | SARS-CoV2 | SARS-CoV1 | S; RBD | B-cells; SARS-CoV2 | 198 | EVQLVESGGGLVQPGGSLRLSCAASGFS |
| | | | | (weak) | | | Human Patient | | VSTKYMTWVRQAPGKGLEWVSVLYSG |
| | | | | | | | | | GSDYYADSVKGRFTISRDNSKNALYLQM |
| | | | | | | | | | NSLRVEDTGVYYCARDSSEVRDHPGHP |
| | | | | | | | | | GRSVGAFDIWGQGTMVTVSS |
| C165 | Ab | SARS-CoV2 | SARS-CoV1 | SARS-CoV2 | SARS-CoV1 | S; RBD | B-cells; SARS-CoV2 | 199 | QVQLVQSGAEVKKPGSSVKVSCKASGG |
| | | | | | | | Human Patient | | TFSSYAINWVRQAPGQGLEWMGRIIPI |
| | | | | | | | | | VGIANYAQKFQGRVTITADKSSSTAYME |
| | | | | | | | | | LSSLRSEDTAVYYCARDLLDPQLDDAFDI |
| | | | | | | | | | WGQGTMVTVSS |
| C201 | Ab | SARS-CoV2 | SARS-CoV1 | | SARS-CoV1, | S; RBD | B-cells; SARS-CoV2 | 200 | EVQLVESGGGLVQPGRSLRLSCAASGFT |
| | | | | | SARS-CoV2 | | Human Patient | | FDDYAMHWVRQAPGKGLEWVSGISW |
| | | | | | | | | | NSGSIGYADSVKGRFTISRDNAKNSLYLQ |
| | | | | | | | | | MNSLRAEDTALYYCVKGVEYSSSSNFDY |
| | | | | | | | | | WGQGTLVTVSS |
| C202 | Ab | SARS-CoV2 | SARS-CoV1 | | SARS-CoV1, | S; RBD | B-cells; SARS-CoV2 | 201 | EVQLVESGGGLVQPGGSLRLSCAASGFT |
| | | | | | SARS-CoV2 | | Human Patient | | VSSNYMSWVRQAPGKGLEWVSLIYSGG |
| | | | | | | | | | STYYADSVKGRFTISRDNSKNTLYLQMN |
| | | | | | | | | | SLRAEDTAVYYCARDTLGRGGDYWGQ |
| | | | | | | | | | GTLVTVSS |
| C204 | Ab | SARS-CoV1, | | | SARS-CoV2 | S; RBD | B-cells; SARS-CoV2 | 202 | EVQLLESGGGLEQPGGSLRLSCAASGFT |
| | | SARS-CoV2 | | | | | Human Patient | | FSTYAMSWVRQAPGKGLEWVSAISGSG |
| | | | | | | | | | AGTFYADSVKGRFTISRDNSKNTLYLQM |
| | | | | | | | | | NSLRAEDTAVYYCARESDCGSTSCYQVG |
| | | | | | | | | | WFDPWGQGTLVTVSS |
| C205 | Ab | SARS-CoV2 | SARS-CoV1 | | SARS-CoV1, | S; non- | B-cells; SARS-CoV2 | 203 | QVQLVQSGAEVKKPGASVKVSCKASGH |
| | | | | | SARS-CoV2 | RBD | Human Patient | | TFTSYYMHWVRQAPGQGLEWMGIINP |
| | | | | | | | | | SGGSTSYAQKFQGRVTMTRDTSTSTVY |
| | | | | | | | | | MELSSLRSEDTAVYYCARGPERGIVGAT |
| | | | | | | | | | DYFDYWGQGTLVTVSS |
| C207 | Ab | SARS-CoV2 | SARS-CoV1 | SARS-CoV2 | SARS-CoV1 | S; RBD | B-cells; SARS-CoV2 | 204 | EVQLLESGGGLVQPGGSLRLSCAASGFT |
| | | | | (weak) | | | Human Patient | | FSSYAMSWVRQAPGKGLEWVSAISGSG |
| | | | | | | | | | GSTYYADSVKGRFTISRDNSKNTLYLQM |
| | | | | | | | | | NSLRAEDTAVYYCAKEPIGQPLLWWDY |
| | | | | | | | | | WGQGTLVTVSS |
| C208 | Ab | SARS-CoV2 | SARS-CoV1 | | SARS-CoV1, | S; non- | B-cells; SARS-CoV2 | 205 | EVQLVQSGAEVKKPGESLKISCKGSGYSF |
| | | | | | SARS-CoV2 | RBD | Human Patient | | TSYWIGWVRQMPGKGLEWMGIIYPGD |
| | | | | | | | | | SDTRYSPSFQGQVTISADKSISTAYLKWS |
| | | | | | | | | | SLKASDSAMYYCARGPNLQNWFDPWG |
| | | | | | | | | | QGTLVTVSS |
| C210 | Ab | SARS-CoV2 | SARS-CoV1 | SARS-CoV2 | SARS-CoV1 | S; RBD | B-cells; SARS-CoV2 | 206 | EVQLVESGGGLIQPGGSLRLSCAASGFT |
| | | | | | | | Human Patient | | VSSNYMSWVRQAPGKGLEWVSVIYSG |
| | | | | | | | | | GSTFYADSVKGRFTFSRDNSKNTLYLQM |
| | | | | | | | | | NSLRAEDTAVYYCARDLMAYGMDVWG |
| | | | | | | | | | QGTTVTVSS |
| C211 | Ab | SARS-CoV2 | SARS-CoV1 | SARS-CoV2 | SARS-CoV1 | S; RBD | B-cells; SARS-CoV2 | 207 | EVQLVESGGGLVQPGGSLRLSCAASEFT |
| | | | | | | | Human Patient | | VSSNYMSWVRQAPGKGLEWVSVIYSG |
| | | | | | | | | | GSTFYADSVKGRFTISRDNSKNTLYLQM |
| | | | | | | | | | NSLRPEDTAVYYCARDYGDFYFDFWGQ |
| | | | | | | | | | GTLVTVSS |
| C212 | Ab | SARS-CoV2 | SARS-CoV1 | | SARS-CoV1, | S; non- | B-cells; SARS-CoV2 | 208 | QVQLVQSGAEVKKPGASVKVSCKASGY |
| | | | | | SARS-CoV2 | RBD | Human Patient | | TVTGYYIHWVRQAPGQGLEWMGWISP |
| | | | | | | | | | NSGGTNYAQKFQGWVTMTRDMSITTA |
| | | | | | | | | | YMELSRLRSDDTAVYYCARERYFDLGG |
| | | | | | | | | | MDVWGQGTTVTVSS |
| C214 | Ab | SARS-CoV2 | SARS-CoV1 | | SARS-CoV1, | S; RBD | B-cells; SARS-CoV2 | 209 | QVQLVESGGGVVQPGRSLRLSCAASGF |
| | | | | | SARS-CoV2 | | Human Patient | | TFSSYGMHWVRQAPGKGLEWVAAIWY |
| | | | | | | | | | DGSNKHYADSVKGRFTISRDNSKNTLYL |
| | | | | | | | | | QMNSLRAEDTAVYYCARDVGRVTTWF |
| | | | | | | | | | DPWGQGTLVTVSS |
| C215 | Ab | SARS-CoV1, | | | SARS-CoV2 | S; RBD | B-cells; SARS-CoV2 | 210 | EVQLLESGGGLVQPGGSLRLSCAASGFT |
| | | SARS-CoV2 | | | | | Human Patient | | FSSYAMSWVRQAPGKGLEWVSAITDSG |
| | | | | | | | | | DGTFYADSVKGRFTISRDNSKNTLYLQM |
| | | | | | | | | | NSLRAEDTAVYYCASEEDYSNYVGWFD |
| | | | | | | | | | PWGQGTLVTVSS |
| C216 | Ab | SARS-CoV2 | SARS-CoV1 | | SARS-CoV1, | S; RBD | B-cells; SARS-CoV2 | 211 | EVQLVESGGGLVQPGGSLRLSCAASGFT |
| | | | | | SARS-CoV2 | | Human Patient | | FSSYDMHWVRQATGKGLEWVSAIGTA |
| | | | | | | | | | GDTYYPDSVKGRFTISRENAKNSLYLQM |
| | | | | | | | | | NSLRAGDTAVYYCARDRGSSGWYGWY |
| | | | | | | | | | FDLWGRGTLVTVSS |
| CA1 | Ab | SARS-CoV2 | | SARS-CoV2 | | S; RBD | B-cells; SARS-CoV2 | 212 | EVQLVQSGAEVKKPGASVKVSCKASGYT |
| | | | | | | | Human Patient | | FTSYGISWVRQAPGQGLEWMGWISAY |
| | | | | | | | | | NGNTNYAQKLQGRVTMTTDTSTSTAY |
| | | | | | | | | | MELRSLRSDDTAVYYCAREGYCSGGSCY |
| | | | | | | | | | SGYYYYYGMDVWGQGTTVTVSS |
| CB6 | Ab | SARS-CoV2 | | SARS-CoV2 | | S; RBD | B-cells; SARS-CoV2 | 213 | EVQLVESGGGLVQPGGSLRLSCAASGFT |
| | | | | | | | Human Patient | | VSSNYMSWVRQAPGKGLEWVSVIYSG |
| | | | | | | | | | GSTFYADSVKGRFTISRDNSMNTLFLQM |
| | | | | | | | | | NSLRAEDTAVYYCARVLPMYGDYLDYW |
| | | | | | | | | | GQGTLVTVSS |
| CC12.1 | Ab | SARS-CoV2 | SARS-CoV1 | SARS-CoV2 | SARS-CoV1 | S; RBD | B-cells; SARS-CoV2 | 214 | EVQLVESGGGLIQPGGSLRLSCAASGLT |
| | | | | | | | Human Patient | | VSSNYMSWVRQAPGKGLEWVSVIYSG |
| | | | | | | | | | GSTFYADSVKGRFTISRDNSKNTLYLQM |
| | | | | | | | | | NSLRAEDTAVYYCARDLDVYGLDVWGQ |
| | | | | | | | | | GTTVTVSS |
| CC12.10 | Ab | SARS-CoV2 | SARS-CoV1 | SARS-CoV2 | SARS-CoV1 | S; RBD | B-cells; SARS-CoV2 | 215 | QVQLVQSGAEVKKPGASVKVSCKASGYI |
| | | | | | | | Human Patient | | YSGYFMHWVRQAPGQGLEWMGWISP |
| | | | | | | | | | DSGGANYAQTFQGRVTMTRDTSTTTAH |
| | | | | | | | | | MELSRLRSDDTAVYYCARGPRYSGTHFD |
| | | | | | | | | | YWGQGTLVTVSS |
| CC12.11 | Ab | SARS-CoV2 | SARS-CoV1 | SARS-CoV2 | SARS-CoV1 | S; RBD | B-cells; SARS-CoV2 | 216 | QVQLVQSGAEVKKPGASVKVSCKASGYI |
| | | | | | | | Human Patient | | FSGYYTHWVRQAPGQGLEWMGWISP |
| | | | | | | | | | DSGGTNYAQKFQGRVTMTRDTSITTAY |
| | | | | | | | | | VELSGLRSDDTAVYYCARGPRYSGTYFD |
| | | | | | | | | | YWGQGTLVTVSS |
| CC12.12 | Ab | SARS-CoV2 | SARS-CoV1 | SARS-CoV2 | SARS-CoV1 | S; RBD | B-cells; SARS-CoV2 | 217 | QVQLVQSGAEVKKPGASVKVSCKASGYI |
| | | | | | | | Human Patient | | YSGYYMHWVRQAPGQGLEWMGWISP |
| | | | | | | | | | DSGGTNYAQRFQGRVTMTRDTSTTTAY |
| | | | | | | | | | MELSRLRSDDTAVYYCARGPRYSGTYFD |
| | | | | | | | | | YWGQGTLVTVSS |
| CC12.13 | Ab | SARS-CoV2 | SARS-CoV1 | SARS-CoV2 | SARS-CoV1 | S; RBD | B-cells; SARS-CoV2 | 218 | EVQLVESGGGLIQPGGSLRLSCAASGFT |
| | | | | | | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAEDTAVYYCARDPYGYSSIWDGQ |
| | | | | | | | | | GGHWGQGTLVTVSS |
| CC12.14 | Ab | SARS-CoV2 | SARS-CoV1 | SARS-CoV2 | SARS-CoV1 | S; RBD | B-cells; SARS-CoV2 | 219 | QVQLVESGGGLVKPGGSLRLSCAASGFT |
| | | | | | | | Human Patient | | FNYYSMNWVRQAPGKGLEWISSISTSSS |
| | | | | | | | | | FVYYADSVKGRFTISRDNAKTSLYLQMN |
| | | | | | | | | | SLRAEDTAVYFCARGGYCSDGSCYVQDR |
| | | | | | | | | | LIYYYSGLDVWGQGTTVTVSS |
| CC12.15 | Ab | SARS-CoV2 | SARS-CoV1 | SARS-CoV2 | SARS-CoV1 | S; RBD | B-cells; SARS-CoV2 | 220 | EVQLVESGGGLVQPGGSLRLSCAASGFT |
| | | | | | | | Human Patient | | FSTYEMNWVRQAPGKGLEWVSYISSSG |
| | | | | | | | | | STIYYADSVKGRFTISRDNAKNSLYLQMS |
| | | | | | | | | | SLRAEDTAIYYCARDRRRRYCTNGVCYR |
| | | | | | | | | | PEEIDYWGQGTLVTVSS |
| CC12.16 | Ab | SARS-CoV2 | SARS-CoV1 | SARS-CoV2 | SARS-CoV1 | S; RBD | B-cells; SARS-CoV2 | 221 | QVQLVESGGGVVQPGRSLRLSCAASGF |
| | | | | (weak) | | | Human Patient | | TFSSYGMHWVRQAPGKGLEWVALIWY |
| | | | | | | | | | DGSNKNYADSVKGRFTISRDNSKNTLDL |
| | | | | | | | | | QMNSLRAEDTAVYYCARDPFPGAVAGT |
| | | | | | | | | | GYLQYWGQGTLVTVSS |
| CC12.17 | Ab | SARS-CoV2 | SARS-CoV1 | SARS-CoV2 | SARS-CoV1 | S; RBD | B-cells; SARS-CoV2 | 222 | EVQLVESGGGVVQPGRSLRLSCAASGFT |
| | | | | (weak) | | | Human Patient | | FRNYGMHWVRQAPGKGLEGVAVISYD |
| | | | | | | | | | GSNKYYADSVKGRFTISRDNSKNTLYLQ |
| | | | | | | | | | MNSLRAEDTAVYYCAKSSGSYYYYYYGM |
| | | | | | | | | | DVWGQGTTVTVSS |
| CC12.18 | Ab | SARS-CoV1, | | SARS-CoV2 | SARS-CoV1 | S; RBD | B-cells; SARS-CoV2 | 223 | EVQLVQSGAEVKKPGASVKVSCKASGYT |
| | | SARS-CoV2 | | (weak) | | | Human Patient | | FTSYYMHWVRQAPGQGLEWMGIINPS |
| | | | | | | | | | GGSTSYAQKFQGRVTMTRDTSTSTVYM |
| | | | | | | | | | ELSSLRSEDTAVYYCARLHCGGDCYLDY |
| | | | | | | | | | WGQGTLVTVSS |
| CC12.19 | Ab | SARS-CoV2 | SARS-CoV1 | SARS-CoV2 | SARS-CoV1 | S; RBD | B-cells; SARS-CoV2 | 224 | EVQLLESGGGLVQPGGSLRLSCAASGFT |
| | | | | (weak) | | | Human Patient | | FSSYAMSWVRQAPGKGLEWVSAISGSG |
| | | | | | | | | | GSTYYADSVKGRFTISRDNSKNTLYLQM |
| | | | | | | | | | NSLRAEDTAVYYCAKGSGSGSYPNYYYY |
| | | | | | | | | | YGMDVWGQGTTVTVSS |
| CC12.2 | Ab | SARS-CoV2 | SARS-CoV1 | SARS-CoV2 | SARS-CoV1 | S; RBD | B-cells; SARS-CoV2 | 225 | EVQLVESGGGLIQPGGSLRLSCAASGFT |
| | | | | | | | Human Patient | | VSSNYMSWVRQAPGKGLEWVSVFYSG |
| | | | | | | | | | GSTYYADSVKGRFTISRDNSKNTLYLQM |
| | | | | | | | | | NSLRAEDTAVYYCARDYGDLYFDYWGQ |
| | | | | | | | | | GTLVTVSS |
| CC12.20 | Ab | SARS-CoV2 | SARS-CoV1 | | SARS-CoV1 | S; non- | B-cells; SARS-CoV2 | 226 | QVQLVESGGGVVQPGRSLRLSCAASGF |
| | | | | | | RBD | Human Patient | | TFSSYGMHWVRQAPGKGLEWVAVISY |
| | | | | | | | | | DGSNKYYADSVKGRFTISRDNSKNTLYL |
| | | | | | | | | | QMSSLRGEDTAVYYCAKDQAYYDILTGY |
| | | | | | | | | | LNPPKNYYYYGMDVWGQGTTVTVSS |
| CC12.21 | Ab | SARS-CoV2 | SARS-CoV1 | | SARS-CoV1 | S; non- | B-cells; SARS-CoV2 | 227 | QVQLVQSGAEVKKPGASVKVSCKVSGY |
| | | | | | | RBD | Human Patient | | TLTELSMHWVRQAPGKGLEWMGGFD |
| | | | | | | | | | PEDGETIYAQKFQGRVTMTEDTSTDTAY |
| | | | | | | | | | MDLSSLRSEDTAVYYCATAFSIFGVVPPD |
| | | | | | | | | | YWGQGTLVTVS |
| CC12.23 | Ab | SARS-CoV2 | SARS-CoV1 | SARS-CoV2 | SARS-CoV1 | S; non- | B-cells; SARS-CoV2 | 228 | QVQLQESGPGLVKPSETLSLTCTVSGGSI |
| | | | | (weak) | | RBD | Human Patient | | SSSSYYWGWIRQPPGKGLEWIGSIYYSG |
| | | | | | | | | | STYYNPSLKSRVTISVDTSKNQFSLKLSS |
| | | | | | | | | | VTAADTAVYYCARQGDCSTTSCAYDYWG |
| | | | | | | | | | QGTLVTVSS |
| CC12.24 | Ab | SARS-CoV2 | SARS-CoV1 | | SARS-CoV1 | S; link | B-cells; SARS-CoV2 | 229 | EVQLVESGGGVVQPGRSLRLSCSASGFT |
| | | | | | | | Human Patient | | FSIYGMHWVRQAPGKGLEWVAVISYD |
| | | | | | | | | | GSNKYYADSVKGRFTISRDNSKNTLYLQ |
| | | | | | | | | | MNSLRAEDTAVYYCAKDRTGNYYYGM |
| | | | | | | | | | DVWGQGTTVTVSS |
| CC12.25 | Ab | SARS-CoV1, | | | SARS-CoV1 | S; non- | B-cells; SARS-CoV2 | 230 | QVQLVESGGGLVQPGGSLRLSCAASGF |
| | | SARS-CoV2 | | | | RBD | Human Patient | | TFSSYAMSWVRQAPGKGLEWVSAISGS |
| | | | | | | | | | GDSTYYADSVKGRFTISRDNSKNTLYLQ |
| | | | | | | | | | MNSLRAEDTAVYYCAKDRYYEFWSGYS |
| | | | | | | | | | NWFDPWGQGTLVTISS |
| CC12.26 | Ab | SARS-CoV2 | SARS-CoV1 | | SARS-CoV1 | S; non- | B-cells; SARS-CoV2 | 231 | EVQLVQSGAEVKKPGESLKISCKGSGYSF |
| | | | | | | RBD | Human Patient | | TSYWIGWVRQMPGKGLEWMGIIYPGD |
| | | | | | | | | | SDTRYSPSFQGQVTVSADKSISTAYLQW |
| | | | | | | | | | SSLKASDTAMYYCARVNYYDSSGYPSFH |
| | | | | | | | | | FDYWGQGTLVTVS |
| CC12.27 | Ab | SARS-CoV1, | | | SARS-CoV1 | S; non- | B-cells; SARS-CoV2 | 232 | QVQLVQSGAEVKKPGASVKVSCKASGY |
| | | SARS-CoV2 | | | | RBD | Human Patient | | TFIGYYMHWVRQAPGQGLEWMGWIN |
| | | | | | | | | | PNSGGTNYAQKFQGRVTMTRDTSISTV |
| | | | | | | | | | YMELSRLRSDDTAVYYCAREMPAAMGY |
| | | | | | | | | | YYYGMDVWGQGTTVTVSS |
| CC12.28 | Ab | SARS-CoV1, | | SARS-CoV2 | | S; RBD | B-cells; SARS-CoV2 | 233 | EVQLLESGGGLVQPGGSLRLSCAASGFT |
| | | SARS-CoV2 | | and SARS- | | | Human Patient | | FSNYAMTWVRQAPGKGLEWVSAISSGS |
| | | | | CoV1 | | | | | GSTYYADSVKGRFTISRDNSKNTVYLQM |
| | | | | | | | | | NSLRAEDTAIYYCAKANKYSSSEFDFWG |
| | | | | | | | | | QGTLVTISS |
| CC12.3 | Ab | SARS-CoV2 | SARS-CoV1 | SARS-CoV2 | SARS-CoV1 | S; RBD | B-cells; SARS-CoV2 | 234 | QVQLVESGGGLIQPGGSLRLSCAASGFT |
| | | | | | | | Human Patient | | VSSNYMSWVRQAPGKGLEWVSVIYSG |
| | | | | | | | | | GSTFYADSVKGRFTISRDNSKSTLYLQM |
| | | | | | | | | | NSLRVEDTAVYYCARDFGDFYFDYWGQ |
| | | | | | | | | | GTLVTVSS |
| CC12.4 | Ab | SARS-CoV2 | SARS-CoV1 | SARS-CoV2 | SARS-CoV1 | S; RBD | B-cells; SARS-CoV2 | 235 | QVQLVQSGAEVKKPGASVKVSCKASGY |
| | | | | | | | Human Patient | | TFTGYYMHWVRQAPGQGLEWMGWIS |
| | | | | | | | | | PNSGGTNYAQKFQGWVTMTRDTSVST |
| | | | | | | | | | AYMELSRLRFDDTAVYYCATESWVYGS |
| | | | | | | | | | GSYSSGAFDIWGQGTMVTVSS |
| CC12.5 | Ab | SARS-CoV2 | SARS-CoV1 | SARS-CoV2 | SARS-CoV1 | S; RBD | B-cells; SARS-CoV2 | 236 | EVQLVQSGAEVKKPGASVKVSCKASGYI |
| | | | | | | | Human Patient | | YSGYYMHWVRQAPGQGLEWMGWISP |
| | | | | | | | | | DSGGTNYAQRFQGRVTMTRDTSTTTAY |
| | | | | | | | | | MELSRLRSDDTAMYYCARGPRYSGTYF |
| | | | | | | | | | DYWGQGTLVTVSS |
| CC12.6 | Ab | SARS-CoV2 | SARS-CoV1 | SARS-CoV2 | SARS-CoV1 | S; RBD | B-cells; SARS-CoV2 | 237 | QVQLVQSGAEVKKPGASVKVSCKASGY! |
| | | | | | | | Human Patient | | FSGYYMHWVRQAPGQGLEWMGWISP |
| | | | | | | | | | DSGGTNYAQKFQGRVTMTRDTSITTGY |
| | | | | | | | | | MELSGLRSDDTAVYYCARGPRYSGTYFD |
| | | | | | | | | | YWGQGTLVTVSS |
| CC12.7 | Ab | SARS-CoV2 | SARS-CoV1 | SARS-CoV2 | SARS-CoV1 | S; RBD | B-cells; SARS-CoV2 | 238 | EVQLVQSGAEVKKPGASVKVTCKTSGYI |
| | | | | | | | Human Patient | | FSGYYMHWVRQVPGQGLEWMGWISP |
| | | | | | | | | | DSGATNYAQKFQGRVTMTRDTSITTSYV |
| | | | | | | | | | ELTWLKSDDTAVYYCARGPRYSGTYFDF |
| | | | | | | | | | WGQGTLVTVSS |
| CC12.8 | Ab | SARS-CoV2 | SARS-CoV1 | SARS-CoV2 | SARS-CoV1 | S; RBD | B-cells; SARS-CoV2 | 239 | QVQLVQSGAEVKKPGASVKVSCKASGYI |
| | | | | | | | Human Patient | | FSGYYTHWVRQAPGQGLEWMGWISP |
| | | | | | | | | | DSGGTNYAQKFQGRVTMTRDTSITTAY |
| | | | | | | | | | VELSGLRSDDTAVYYCARGPRYSGTYFD |
| | | | | | | | | | YWGQGTLVTVSS |
| CC12.9 | Ab | SARS-CoV2 | SARS-CoV1 | SARS-CoV2 | SARS-CoV1 | S; non- | B-cells; SARS-CoV2 | 240 | EVQLVQSGAEVKKPGASVKVSCKASGYI |
| | | (weak) | | | | RBD | Human Patient | | FSGYYMHWVRQAPGQGLEWMGWISP |
| | | | | | | | | | DSGGTNYAQNFQGRVTMTRDTSISTGY |
| | | | | | | | | | MELSRLRSDDTAMYYCARGPRYSGTYF |
| | | | | | | | | | DYWGQGVLVTVSS |
| CC6.29 | Ab | SARS-CoV2 | | SARS-CoV2 | | S; RBD | B-cells; SARS-CoV2 | 241 | QVQLVQSGSELKKPGASVKVSCKASGYT |
| | | | | | | | Human Patient | | FATYALNWVRQAPGQGLEWMGWVNT |
| | | | | | | | | | NTGSPTYAQGFTGRFVFSFDTSVSTAYL |
| | | | | | | | | | QIRTLKAEDTAVYYCAVYYYDSGSPGWF |
| | | | | | | | | | DPWGQGTLVTVSS |
| CC6.30 | Ab | SARS-CoV2 | | SARS-CoV2 | | S; RBD | B-cells; SARS-CoV2 | 242 | QVQLVQSGAEVKKPGSSVKVSCKASGG |
| | | | | | | | Human Patient | | TFSIYAITWVRQAPGQGLEWMGGIIPII |
| | | | | | | | | | GTANYAQKFQGRVTITADKSTSTAYMEL |
| | | | | | | | | | SSLRSEDTAVYYCARDFRYCSSTRCYFWF |
| | | | | | | | | | DPWGQGTLVTVSS |
| CC6.31 | Ab | SARS-CoV2 | | SARS-CoV2 | | S; RBD | B-cells; SARS-CoV2 | 243 | EVQLVQSGAEVKKPGASVKVSCMASGY |
| | | | | | | | Human Patient | | TFTSYYMHWVRQAPGQGLEWMGIISP |
| | | | | | | | | | SGGGTSYAQKFQGRVTLTRDTSTSTVYM |
| | | | | | | | | | ELSSLRSEDTAVYYCARWYDSTGSIDYW |
| | | | | | | | | | GQGTLVTVSS |
| CC6.32 | Ab | SARS-CoV1, | | SARS-CoV2 | | S; RBD | B-cells; SARS-CoV2 | 244 | EVQLVESGGGLVQPGRSLRLSCAASGFT |
| | | SARS-CoV2 | | | | | Human Patient | | FDDYAMHWVRQAPGKGLEWVSGISW |
| | | | | | | | | | NSGSIAFAGSVKGRFTISRDNAKNSLYLQ |
| | | | | | | | | | MNSLRAEDTALYYCAKDQGYSYGNYFD |
| | | | | | | | | | YWGQGTLVTVSS |
| CC6.33 | Ab | SARS-CoV1, | | SARS-CoV2 | | S; RBD | B-cells; SARS-CoV2 | 245 | QVQLVQSGAEVKKPGSSVKVSCKASGG |
| | | SARS-CoV2 | | and SARS- | | | Human Patient | | TFSSSAISWVRQAPGQGLEWMGGIIPIL |
| | | | | CoV1 | | | | | DITNYAQKFQGRVTITADKSTSTAFMELS |
| | | | | | | | | | SLRSEDTAVYYCALRNQWDLLVYWGQG |
| | | | | | | | | | TLVTVSS |
| Clone11- | Ab | SARS-CoV2 | | | | S; RBD | Immunised Mouse | 246 | QVQLQQPGAELVMPGASVKMSCKASG |
| 9 | | | | | | | | | YTFTDYWMHWVKQRPGQGLEWIGAE |
| | | | | | | | | | DTSDSYTSYNQKFKGKATLTVDESSSTAY |
| | | | | | | | | | MQLSSLTSEDSAVYYCARRGYGSSYTWF |
| | | | | | | | | | AYWGQGTLVTVSA |
| CnC2t1p1_ | Ab | SARS-CoV2 | | | SARS-CoV2 | S; link | B-cells; SARS-CoV2 | 247 | QVQLVQSGAEVKKPGSSVKVSCKASGG |
| B10 | | | | | | | Human Patient | | TFSSYAISWVRQAPGQGLEWMGGIIPIF |
| | | | | | | | | | GTANYAQKFQGRVTITADKSTSTAYMEL |
| | | | | | | | | | SSLRSEDTAVYYCARVSGYDSSGYWGDY |
| | | | | | | | | | WGQGTLVTVSS |
| CnC2t1p1_ | Ab | SARS-CoV2 | | SARS-CoV2 | | S; RBD | B-cells; SARS-CoV2 | 248 | QVQLVQSGAEVKKPGASVKVSCKASGY |
| B4 | | | | | | | Human Patient | | TFTSYGISWVRQAPGQGLEWMGWISA |
| | | | | | | | | | YNGNTNYAQKLQGRVTMTTDTSTSTAY |
| | | | | | | | | | MELRSLRSDDTAVYYCARDGELLGWFD |
| | | | | | | | | | PWGQGTLVTVSS |
| CnC2t1p1_ | Ab | SARS-CoV2 | | SARS-CoV2 | | S; RBD | B-cells; SARS-CoV2 | 249 | EVQLVESGGGLVQPGRSLRLSCTASGFT |
| D6 | | | | | | | Human Patient | | FGDYAMSWFRQAPGKGLEWVGFIRSK |
| | | | | | | | | | AYGGTTEYAASVKGRFTISRDDSKSIAYL |
| | | | | | | | | | QMNSLKTEDTAVYYCTRVRRLWFGSYY |
| | | | | | | | | | YGMDVWGQGTTVTVSS |
| CnC2t1p1_ | Ab | SARS-CoV2 | | SARS-CoV2 | | S; RBD | B-cells; SARS-CoV2 | 250 | EVQLVESGGGLVQPGRSLRLSCTASGFT |
| E12 | | | | | | | Human Patient | | FGDYAMSWFRQAPGKGLEWVGFIRSK |
| | | | | | | | | | AYGGTTEYAASVKGRFTISRDDSKSIVYL |
| | | | | | | | | | QMNSLKTEDTAVYYCTRVRRLWFGSYY |
| | | | | | | | | | YGMDVWGQGTTVTVSS |
| CnC2t1p1_ | Ab | SARS-CoV2 | | SARS-CoV2 | | S; RBD | B-cells; SARS-CoV2 | 251 | QVQLVQSGAEVKNPGASVKVSCKASGY |
| E8 | | | | | | | Human Patient | | IFTNYYIHWVRQAPGQGLEWVGWIHSL |
| | | | | | | | | | SGGTSYAQKFQGRVTLTRDASIRTAYME |
| | | | | | | | | | LSRLGSDDTALYYCARASVSTITDFDYWG |
| | | | | | | | | | QGTLVAVSS |
| CnC2t1p1_ | Ab | SARS-CoV2 | | SARS-CoV2 | | S; RBD | B-cells; SARS-CoV2 | 252 | QVQLVQSGAEVKKPGASVKVSCKASGYI |
| G6 | | | | | | | Human Patient | | FTNYYIHWVRQAPGQGLEWVGWIHSL |
| | | | | | | | | | SGGTSYAQKFQGRVTLTRDAPIRTAYME |
| | | | | | | | | | LSGLGSDDTAVYYCARASVATITDFDYW |
| | | | | | | | | | GQGTLVAVSS |
| COV2- | Ab | SARS-CoV1, | | | SARS-CoV2 | S; non- | B-cells; SARS-CoV2 | 253 | QVQLVESGGGVVQPGRSLRLSCAASGF |
| 2006 | | SARS-CoV2 | | | | RBD | Human Patient | | TFSYYAILWFRQAPGKGLEWVAIISYDGS |
| | | | | | | | | | NKYYADSVKGRFTISRDNSKNTLYLQMN |
| | | | | | | | | | SLRPEDTAVYYCARPQSGGYYAPLDYW |
| | | | | | | | | | GQGTLVTVSS |
| COV2- | Ab | SARS-CoV1, | | | SARS-CoV2 | S; RBD | B-cells; SARS-CoV2 | 254 | QVQLVESGGGVVQPGRSLRLSCAASGF |
| 2007 | | SARS-CoV2 | | | | | Human Patient | | TFSRYGMHWVRQAPGKGLEWVGIISYD |
| | | | | | | | | | ASDKTYAESVKGRFTISRDNSKNTLYLQ |
| | | | | | | | | | MNSLRAEDTAVYYCAKVSATYYYYYYG |
| | | | | | | | | | MDVWGQGTTVTVSS |
| COV2- | Ab | SARS-CoV1, | | | SARS-CoV2 | S; non- | B-cells; SARS-CoV2 | 255 | QVQLVESGGGVVQPGRSLRLSCAASGF |
| 2009 | | SARS-CoV2 | | | | RBD | Human Patient | | TFSTYAMHWVRQAPGKGLEWVAVISY |
| | | | | | | | | | DGSNKYYADSVKGRFTISRDNPKNTLYL |
| | | | | | | | | | QMNSLRAEDTAVYYCARDTATYVLLWS |
| | | | | | | | | | GDFNLDYWGQGTLVTVSS |
| COV2- | Ab | SARS-CoV1, | | | SARS-CoV2 | S; non- | B-cells; SARS-CoV2 | 256 | QVQLVESGGGVVQPGRSLRLSCAASGF |
| 2011 | | SARS-CoV2 | | | | RBD | Human Patient | | TFSTYAMHWVRQAPGKGLEWVTLISYD |
| | | | | | | | | | GGNKYYADSVKGRFTISRDNSKNTLYLQ |
| | | | | | | | | | MNSLRAEDAAVYFCARGHTGNYYYGM |
| | | | | | | | | | DVWGQGTTVTVSS |
| COV2- | Ab | SARS-CoV1, | | | SARS-CoV2 | S; non- | B-cells; SARS-CoV2 | 257 | QVHLVESGGGVVQPGRSLRLSCAASGF |
| 2013 | | SARS-CoV2 | | | | RBD | Human Patient | | TFSNYGMHWVRQAPGKGLEWVAVISG |
| | | | | | | | | | DENNKFYANSVKGRFTISRDNSKNTLSL |
| | | | | | | | | | QMNSLRPEDTARYYCAKGGDSSGWAW |
| | | | | | | | | | DGDNPPTDYWGQGTLVIVSS |
| COV2- | Ab | SARS-CoV1, | | SARS-CoV2 | | S; RBD | B-cells; SARS-CoV2 | 258 | EVQLVESGGGLVQPGRSLRLSCAASGFT |
| 2015 | | SARS-CoV2 | | (weak) | | | Human Patient | | FDDYAMHWVRQAPGKGLEWVSGISW |
| | | | | | | | | | NSGSIGYADSVKGRFTISRDNAKNSLYLQ |
| | | | | | | | | | MNSLRTEDTALYYCAMGPFGELLPYYFD |
| | | | | | | | | | YWGQGTLVTVSS |
| COV2- | Ab | SARS-CoV1, | | | SARS-CoV2 | S; RBD | B-cells; SARS-CoV2 | 259 | QVQLVQSGAEVKKPGASVKVSCKVSGY |
| 2016 | | SARS-CoV2 | | | | | Human Patient | | TLTELSIHWVRQAPGKGLEWMGGFDP |
| | | | | | | | | | EDAETIYAQNFQGRVTMTEDTSTDTAY |
| | | | | | | | | | MELSSLRSEDTALYYCAAAPAVMTAGW |
| | | | | | | | | | FDPWGQGTLVSVSS |
| COV2- | Ab | SARS-CoV1, | | | SARS-CoV2 | S; non- | B-cells; SARS-CoV2 | 260 | QVQLEESGPGLVKPSETLSLTCTVSGGSI |
| 2017 | | SARS-CoV2 | | | | RBD | Human Patient | | SSHYWSWIRQPPGKGLEWIGYIQDSGS |
| | | | | | | | | | TNYNPSLKSRVTISVDTSKNQFSLRLSSV |
| | | | | | | | | | TTADTAVYYCVRGAMAWFDPWGQGTL |
| | | | | | | | | | VTVSS |
| COV2- | Ab | SARS-CoV2 | SARS-CoV1 | | SARS-CoV1, | S; NTD | B-cells; SARS-CoV2 | 261 | QVQLVQSGAEVKKPGASVKVSCKVSGY |
| 2021 | | | | | SARS-CoV2 | | Human Patient | | TLIELSIHWVRQAPGKGLEWMGGFDPE |
| | | | | | | | | | DVETIYAQKFQGRVTMTEDTSTDTAYM |
| | | | | | | | | | ELSSLTSEDRAVYYCATQPAAIGGTPPYY |
| | | | | | | | | | WGQGTLVTVSS |
| COV2- | Ab | SARS-CoV2 | SARS-CoV1 | | SARS-CoV1, | S; non- | B-cells; SARS-CoV2 | 262 | QVQLVQSGAEVKKPGASVKVSCKASGY |
| 2022 | | | | | SARS-CoV2 | RBD | Human Patient | | TFTSYVISWVRQAPGQGLEWMGWISA |
| | | | | | | | | | YNGNTNYAQKLQGRVTMTTDTSTSTAY |
| | | | | | | | | | MELRSLRSDDTAVYYCARDQGPTYYYGS |
| | | | | | | | | | GSPHYGMDVWGQGTTVTVSS |
| COV2- | Ab | SARS-CoV1, | | | SARS-CoV2 | S; non- | B-cells; SARS-CoV2 | 263 | QVHLVESGGGVVQPGRSLRLSCAASGF |
| 2025 | | SARS-CoV2 | | | | RBD | Human Patient | | TFSNYGMHWVRQAPGKGLEWVAVISG |
| | | | | | | | | | DENNKFYANSVKGRFTISRDNSKNTLSL |
| | | | | | | | | | QMNSLRPEDTARYYCAKGGDSSGWAW |
| | | | | | | | | | DGDNPPTDYWGQGTLVIVSS |
| COV2- | Ab | SARS-CoV2 | SARS-CoV1 | | SARS-CoV1, | S; NTD | B-cells; SARS-CoV2 | 264 | QVQLVQSGAEVKKPGASVKVSCKVSGY |
| 2026 | | | | | SARS-CoV2 | | Human Patient | | TLTELSIHWVRQAPGKGLEWMGGFDP |
| | | | | | | | | | EDGETVYAQKFQGRVTMTEDTSSDTAY |
| | | | | | | | | | MELSSLRSEDTAVYYCATSFPIRGDPSYY |
| | | | | | | | | | YYYYGMDVWGQGTTVTVSS |
| COV2- | Ab | SARS-CoV2 | SARS-CoV1 | | SARS-CoV1, | S; non- | B-cells; SARS-CoV2 | 265 | QVQLVESGGGVVQPGRSLRLSCAASGFI |
| 2027 | | | | | SARS-CoV2 | RBD | Human Patient | | FSTYGMHWVRQAPGKGLEWVAVISYD |
| | | | | | | | | | GSNKYNADSVKGRFTISRDNSKNTLYLQ |
| | | | | | | | | | MNSLRVEDTAVYYCAIYGYYYYGLDVW |
| | | | | | | | | | GQGTTVTVSS |
| COV2- | Ab | SARS-CoV1, | | | SARS-CoV2 | S; non- | B-cells; SARS-CoV2 | 266 | QVHLVESGGGVVQPGRSLRLSCAASGF |
| 2028 | | SARS-CoV2 | | | | RBD | Human Patient | | SFRNYGMHWVRQAPGKGLEWVAVISG |
| | | | | | | | | | DENNKFYANSVKGRFTISRDNSKNTLSL |
| | | | | | | | | | QMNSLRPEDTARYYCAKGGDSSGWAW |
| | | | | | | | | | DGDNPPTDYWGQGTLVIVSS |
| COV2- | Ab | SARS-CoV1, | | | SARS-CoV2 | S; non- | B-cells; SARS-CoV2 | 267 | QVQLQESGPGLVKPSGTLSLTCVVSGGS |
| 2029 | | SARS-CoV2 | | | | RBD | Human Patient | | ISSSNWGWVRQPPGKGLEWIGEIYLSG |
| | | | | | | | | | TTNYNPSLTSRVTISVDKSKNQFSLKLNS |
| | | | | | | | | | VTAADTAIYYCARPTAGAGGAFDIWGQ |
| | | | | | | | | | GTVVTVSS |
| COV2- | Ab | SARS-CoV1, | | | SARS-CoV2 | S; RBD | B-cells; SARS-CoV2 | 268 | QVTLKESGPVLVKPTETLTLTCTVSGFSL |
| 2031 | | SARS-CoV2 | | | | | Human Patient | | SNARMGVSWIRQPPGKALEWLAHIFWN |
| | | | | | | | | | DENSYSTSLKTRLTISKDTSKSQVVLNMT |
| | | | | | | | | | NMDPVDTATYYCARTEWLLSDNWFDS |
| | | | | | | | | | WGQGTLVTVSS |
| COV2- | Ab | SARS-CoV1, | | | SARS-CoV2 | S; RBD | B-cells; SARS-CoV2 | 269 | QVQLVQSGAEVKKPGASVKVSCKASGD |
| 2032 | | SARS-CoV2 | | | | | Human Patient | | TFSSYYLHWVRQAPGQGLQWMGISNP |
| | | | | | | | | | SGGSTTYAQKFQGRVTMTGDTSTSTVY |
| | | | | | | | | | MELSSLRSEDTAVYYCARGGLVPAARNA |
| | | | | | | | | | FDIWGQGTMVTVSS |
| COV2- | Ab | SARS-CoV1, | | | SARS-CoV2 | S; non- | B-cells; SARS-CoV2 | 270 | QVQLQESGPGLVKPSGTLSLTCVVSGGS |
| 2033 | | SARS-CoV2 | | | | RBD | Human Patient | | ISSSNWGWVRQPPGKGLEWIGEIYHSG |
| | | | | | | | | | TTNYNPSLRSRVTISVDKSKNQLSLKLNS |
| | | | | | | | | | VTAADTAIYYCARPTAGAGGAFDTWGQ |
| | | | | | | | | | GTMVTVSA |
| COV2- | Ab | SARS-CoV1, | | | SARS-CoV2 | S; non- | B-cells; SARS-CoV2 | 271 | QVQLVQSGAEVKKPGASVKVSCKASGY |
| 2034 | | SARS-CoV2 | | | | RBD | Human Patient | | TFTTYGISWVRQAPGQGLEWMGWISA |
| | | | | | | | | | YNGNSNYGKKFQGRVTMTADTSTSTAY |
| | | | | | | | | | MELRSLRSDDTAVYYCARDLPIKVVVPA |
| | | | | | | | | | ADYNWFDPWGQGTLVTVSS |
| COV2- | Ab | SARS-CoV2 | SARS-CoV1 | | SARS-CoV1, | S; non- | B-cells; SARS-CoV2 | 272 | QITLKESGPTLVKPTQTLTLTCTFSGFSL |
| 2035 | | | | | SARS-CoV2 | RBD | Human Patient | | NTNGVAVGWIRQPPGKALEWLALIYWD |
| | | | | | | | | | DDKRYSPSLKSRLTITKDTSKNQVVLTMT |
| | | | | | | | | | NMDPVDTATYYCAHRRGILTEDAFDIW |
| | | | | | | | | | GQGTMVTVSS |
| COV2- | Ab | SARS-CoV2 | SARS-CoV1 | SARS-CoV2 | | S; RBD | B-cells; SARS-CoV2 | 273 | EVQLVESGGGLVQPGESLRLSCAASGIT |
| 2037 | | | | | | | Human Patient | | VSSNYMSWVRQAPGKGLEWVSVIYSG |
| | | | | | | | | | GSTFYADSVKGRFIISRHNSKNTLYLQM |
| | | | | | | | | | NSLRAEDTAVYYCARDLNEHGLDVWG |
| | | | | | | | | | QGTTVSVSS |
| COV2- | Ab | SARS-CoV2 | SARS-CoV1 | | SARS-CoV1, | S; RBD | B-cells; SARS-CoV2 | 274 | QVQLVQSGAEVKKPGASVKVSCKASGY |
| 2039 | | | | | SARS-CoV2 | | Human Patient | | TFTSYYMHWVRQAPGQGLEWMGIINP |
| | | | | | | | | | SAGSTSYAQKFQGRVTMTRDTSTSTVY |
| | | | | | | | | | MELSSLRSEDTAVYYCARGTLIPAHRGAF |
| | | | | | | | | | DIWGQGTMVTVSS |
| COV2- | Ab | SARS-CoV1, | | | SARS-CoV2 | S; non- | B-cells; SARS-CoV2 | 275 | QVQLQESGPGLVKPSQTLSLTCTVSGGSI |
| 2041 | | SARS-CoV2 | | | | RBD | Human Patient | | SSGTYYWSWIRQPAGKGLEWIGRFYTS |
| | | | | | | | | | GSTNYNPSLKSRVTISVDASKNQFSLKLS |
| | | | | | | | | | SVTAADTAVYYCARARPDYYYYYAMDV |
| | | | | | | | | | WGQGTTVTVSS |
| COV2- | Ab | SARS-CoV2 | SARS-CoV1 | SARS-CoV2 | | S; RBD | B-cells; SARS-CoV2 | 276 | EVQLVESGGGLVQPGRSLRLSCAASGFT |
| 2046 | | | | | | | Human Patient | | FDDYAMHWVRQAPGKGLEWVSGISW |
| | | | | | | | | | NSGSIAYTDSVKGRFTISRDNAKNSLYLQ |
| | | | | | | | | | MNSLRAEDTALYYCAKAHSTGHQYYYG |
| | | | | | | | | | MDVWGQGTTVTVSS |
| COV2- | Ab | SARS-CoV2 | SARS-CoV1 | SARS-CoV2 | | S; RBD | B-cells; SARS-CoV2 | 277 | QVQLVQSGAEVKKPGASVKVSCKASGY |
| 2050 | | | | | | | Human Patient | | TFTDYYMHWVRQAPGQGLEWMGWI |
| | | | | | | | | | NPNSRGTNYAQKFQGRVTMTRDTSIST |
| | | | | | | | | | VYMELSRLTSDDTAVYYCARVVVLGYGR |
| | | | | | | | | | PNNYYDGRNVWDYWGQGTLVTVSS |
| COV2- | Ab | SARS-CoV2 | SARS-CoV1 | | SARS-CoV1, | S; RBD | B-cells; SARS-CoV2 | 278 | QVTLRESGPALVKPTQTLSLTCTFSGFSL |
| 2051 | | | | | SARS-CoV2 | | Human Patient | | GTSGMCVSWIRQPPGKALEWLARIDW |
| | | | | | | | | | DDDKYYSTSLKTRLTISKDTSKNQVVLTM |
| | | | | | | | | | TNMDPVDTATYYCARGVVTYDYWGQG |
| | | | | | | | | | TLVTVSS |
| COV2- | Ab | SARS-CoV2 | SARS-CoV1 | | SARS-CoV1, | S; non- | B-cells; SARS-CoV2 | 279 | QVQLVQSGAEVKKPGSSVKVSCKASGD |
| 2054 | | | | | SARS-CoV2 | RBD | Human Patient | | TFSSYTINWVRQAPGQGLEWMGRIIPIL |
| | | | | | | | | | GIPNYAQKFQGRVTITADKSTSTAFMEL |
| | | | | | | | | | SSLRSEDTAVYYCARGRGYSNYGASYYM |
| | | | | | | | | | DVWGKGTTVTVSS |
| COV2- | Ab | SARS-CoV1, | | | SARS-CoV2 | S; RBD | B-cells; SARS-CoV2 | 280 | EVQLVESGGGLVQPGRSLRLSCAASGFT |
| 2055 | | SARS-CoV2 | | | | | Human Patient | | FDDYAMNWVRQPPGKGLEWVSGISW |
| | | | | | | | | | NSDSIGYADSVKGRFTISRDNAKNSLYLQ |
| | | | | | | | | | MNSLRAEDTAMYYCAKGRGAGYTSYM |
| | | | | | | | | | DVWGKGTTVTVSS |
| COV2- | Ab | SARS-CoV2 | SARS-CoV1 | SARS-CoV2 | | S; RBD | B-cells; SARS-CoV2 | 281 | QVQLAQSGAEVKKPGASVKVSCKAAGY |
| 2064 | | | | | | | Human Patient | | TFTSYDINWVRQATGQGLEWMGWMN |
| | | | | | | | | | SNSGNAGYAQKFQGRVTMTRDTSTSTA |
| | | | | | | | | | YMELSSLTSDDTAVYYCARMRTGWPTH |
| | | | | | | | | | GRPDDFWGRGTLVTVSS |
| COV2- | Ab | SARS-CoV2 | SARS-CoV1 | SARS-CoV2 | | S; RBD | B-cells; SARS-CoV2 | 282 | EVQLVESGGGLVQPGGSLRLSCAASGFT |
| 2068 | | | | | | | Human Patient | | VSSNYMSWVRQAPGKGLEWVSVIYPG |
| | | | | | | | | | GSAFYADSVKGRFTISRHNSNNTLCLQM |
| | | | | | | | | | NSLRTEDTAVYYCARSYDILTGYRDAFDI |
| | | | | | | | | | WGQGTMVTVSS |
| COV2- | Ab | SARS-CoV2 | SARS-CoV1 | | SARS-CoV1, | S; RBD | B-cells; SARS-CoV2 | 283 | QVQLQQWGAGLLKPSETLSLTCAVSGG |
| 2070 | | | | | SARS-CoV2 | | Human Patient | | SFSAYYWSWIRQPPGKGLEWIGEINHS |
| | | | | | | | | | GSTNYNPSLRSRVTISVDTSKNQFSLKLS |
| | | | | | | | | | SVTAADTAVYYCARVGYSQGYYYYYMDV |
| | | | | | | | | | WGKGTTVTVSS |
| COV2- | Ab | SARS-CoV2 | SARS-CoV1 | SARS-CoV2 | | S; RBD | B-cells; SARS-CoV2 | 284 | QMQLVQSGPEVKKPGTSVKVSCKTSGF |
| 2072 | | | | | | | Human Patient | | TFTSSAIQWVRQARGQRLEWIGWIVVG |
| | | | | | | | | | SGNTNYAQKFQERVTITRDMSTSTAYM |
| | | | | | | | | | ELSSLRSEDTAVYYCAAPHCNRTSCYDAF |
| | | | | | | | | | DLWGQGTMVTVSS |
| COV2- | Ab | SARS-CoV2 | SARS-CoV1 | | SARS-CoV1, | S; RBD | B-cells; SARS-CoV2 | 285 | QVQLVQSGAEVKKPGSSVKVSCKASGG |
| 2078 | | | | | SARS-CoV2 | | Human Patient | | TFSSYSITWVRQAPGQGLEWMGRIIPVL |
| | | | | | | | | | GIANYAQKFQDRVTITADKSTSTAYMEL |
| | | | | | | | | | SSLRSEDTAVYYCARVGVSGFKSGSNWY |
| | | | | | | | | | FDLWGRGTLVTVSS |
| COV2- | Ab | SARS-CoV2 | SARS-CoV1 | | SARS-CoV1, | S; RBD | B-cells; SARS-CoV2 | 286 | EVQLVESGGGLVQPGGSLRLSCAASGLT |
| 2080 | | | | | SARS-CoV2 | | Human Patient | | VRSNYMTWVRQTPGKGLEWVSVIYSG |
| | | | | | | | | | GSTFYADSVKGRFTISRDNSKNTVYLQM |
| | | | | | | | | | NSLRAEDTAVYYCARDLVTYGLDVWGQ |
| | | | | | | | | | GTTVTVSS |
| COV2- | Ab | SARS-CoV1, | | | SARS-CoV2 | S; RBD | B-cells; SARS-CoV2 | 287 | EVQLVESGGGVVRPGGSLRLSCAASGFI |
| 2082 | | SARS-CoV2 | | | | | Human Patient | | FDDYDMTWVRQAPGKGLEWVSGISW |
| | | | | | | | | | NGGNTGYADSVKGRFTISRDNAKNSLYL |
| | | | | | | | | | QMNSLRAEDTALYHCAVIMSPIPRYSGY |
| | | | | | | | | | DWAGGAFDIWGQGTMVTVSS |
| COV2- | Ab | SARS-CoV1, | | | SARS-CoV2 | S; RBD | B-cells; SARS-CoV2 | 288 | QVQLVESGGGVVQPGRSLRLSCAASGF |
| 2083 | | SARS-CoV2 | | | | | Human Patient | | TFSNYGMHWVRQAPGKGLEWVAVMS |
| | | | | | | | | | YDGSNKYYADSVKGRFTISRDNSKNTLYL |
| | | | | | | | | | QMNSLRAEDTAVYYCAKNLGPYCSGGT |
| | | | | | | | | | CYSLVGDYWGQGTLVTVSS |
| COV2- | Ab | SARS-CoV1, | | | SARS-CoV2 | S; RBD | B-cells; SARS-CoV2 | 289 | EVQLVESGGGVVRPGGSLRLSCAASGFI |
| 2094 | | SARS-CoV2 | | | | | Human Patient | | FDDYDMTWVRQAPGKGLEWVSGISW |
| | | | | | | | | | NGGSTGYADSVKGRFTISRDNAKNSLYL |
| | | | | | | | | | QMSSLRAEDTALYHCAVIMSPIPRYSGY |
| | | | | | | | | | DWAGDAFDIWGQGTMVTVSS |
| COV2- | Ab | SARS-CoV2 | SARS-CoV1 | SARS-CoV2 | | S; RBD | B-cells; SARS-CoV2 | 290 | QVQLVQSGAEVKKPGASVKVSCKASGY |
| 2096 | | | | | | | Human Patient | | TFGSFDISWVRQATGQGLEWMGRMS |
| | | | | | | | | | SSSGSTAYAQKFQGRVTMTRDTSTSTA |
| | | | | | | | | | YMELSSLRSEDTAMYYCARMRSGWPTH |
| | | | | | | | | | GRPDDFWGRGTLVTVSS |
| COV2- | Ab | SARS-CoV2 | SARS-CoV1 | SARS-CoV2 | | S; RBD | B-cells; SARS-CoV2 | 291 | EVQLVESGGGLVQPGRSLRLSCAASGFT |
| 2097 | | | | | | | Human Patient | | FDDYAMHWVRQAPGKGLEWVSGISW |
| | | | | | | | | | SSGTIGYADSVKGRFIISRDSAKSSLYLQ |
| | | | | | | | | | MSSLRPEDTALYYCAKDIIRQGEDGMD |
| | | | | | | | | | VWGQGTTVTVSS |
| COV2- | Ab | SARS-CoV2 | SARS-CoV1 | SARS-CoV2 | | S; RBD | B-cells; SARS-CoV2 | 292 | EVQLLESGGGLIQPGGSLRLSCAASGFTF |
| 2098 | | | | | | | Human Patient | | SSYAMSWVRQAPGKGLEWVSGIISTSG |
| | | | | | | | | | GATYSADSVRGRFTTSRDSSKSILYLQM |
| | | | | | | | | | SSLRGEDTAVYYCVKGLFDWFPLWGQ |
| | | | | | | | | | GTMVTVSS |
| COV2- | Ab | SARS-CoV1, | | SARS-CoV2 | | S; RBD | B-cells; SARS-CoV2 | 293 | EVQLVESGGGLVQPGGSLRLSCAASGFT |
| 2103 | | SARS-CoV2 | | | | | Human Patient | | FSRHWMTWVRQAPGKGLEWVASIKQ |
| | | | | | | | | | DGSEKYYVDSVKGRLTISRDSAKSSLYL |
| | | | | | | | | | QMSSLRAEDTAVYYCARLGFYYGGADY |
| | | | | | | | | | WGQGTLVTVSS |
| COV2- | Ab | SARS-CoV2 | SARS-CoV1 | | SARS-CoV1, | S; RBD | B-cells; SARS-CoV2 | 294 | SVQLVESGGGLVQPGGSLRLSCAASGF |
| 2108 | | | | | SARS-CoV2 | | Human Patient | | TFHHYAMHWVRQAPGKGLEWVSGISG |
| | | | | | | | | | SSDYRAYADSLKGRFTISRDYAKSSLWL |
| | | | | | | | | | QMSSLTSEDTAFYYCAKGVDYGGKLAYF |
| | | | | | | | | | DSWGQGTLVTVSS |
| COV2- | Ab | SARS-CoV2 | SARS-CoV1 | | SARS-CoV1, | S; RBD | B-cells; SARS-CoV2 | 295 | QVQLVESGGGVVQPGRSLRLSCAASGF |
| 2110 | | | | | SARS-CoV2 | | Human Patient | | SFSSYVMSWVRQAPGKGqLEWVAVISY |
| | | | | | | | | | DGSSKYYADSVKGRFTISRDSSKSTLYLQ |
| | | | | | | | | | MSSLRAEDTAVYYCARDIDSGYDPTPVF |
| | | | | | | | | | DYWGQGTLVTVSS |
| COV2- | Ab | SARS-CoV1, | | | SARS-CoV2 | S; RBD | B-cells; SARS-CoV2 | 296 | EVQLVESGGGLVQPGGSLRLSCAASGFT |
| 2111 | | SARS-CoV2 | | | | | Human Patient | | FSSYDLHWVRQGTGKRLEWVSAIGTAG |
| | | | | | | | | | DTYYLGSVKGRFTISRESAKSSLYLQMS |
| | | | | | | | | | SLRAGDTAVYYCARVLYDSSGFYSWFDP |
| | | | | | | | | | WGQGTLVTVSS |
| COV2- | Ab | SARS-CoV2 | SARS-CoV1 | SARS-CoV2 | | S; RBD | B-cells; SARS-CoV2 | 297 | EVQLVESGGGLIQPGGSLRLSCAASEVT |
| 2113 | | | | | | | Human Patient | | VSSSYMSWVRQAPGKGLEWVSLIYSGG |
| | | | | | | | | | TTYYADSVKGRFTISRDSSKSTLYLQMS |
| | | | | | | | | | SLRAEDTAVYYCARDFLRWHDLWGQG |
| | | | | | | | | | TLVTVSS |
| COV2- | Ab | SARS-CoV2 | SARS-CoV1 | | SARS-CoV1, | S; RBD | B-cells; SARS-CoV2 | 298 | QVQLVQSGAEVKKPGSSVKVSCKASGD |
| 2114 | | | | | SARS-CoV2 | | Human Patient | | TFSSYTINWVRQAPGQGLEWMGRIIPIL |
| | | | | | | | | | GIPNYAQKFQGRVTITADKSTSTAFMEL |
| | | | | | | | | | SSLRSEDTAVYYCARGRGYSNYGASYYM |
| | | | | | | | | | DVWGKGTTVTVSS |
| COV2- | Ab | SARS-CoV1, | | | SARS-CoV2 | S; RBD | B-cells; SARS-CoV2 | 299 | QLQLQESGPGLVKPSETLSLTCTVSGGSI |
| 2128 | | SARS-CoV2 | | | | | Human Patient | | SSSSYYWGWIRQPPGNGLEWIGSIYYSG |
| | | | | | | | | | STYYNPSLKGRVSISVDTSKNQFSLKLSS |
| | | | | | | | | | VTAADTAVYYCARILVIFTLNWFDPWGQ |
| | | | | | | | | | GTLVTVSS |
| COV2- | Ab | SARS-CoV2 | SARS-CoV1 | SARS-CoV2 | | S; RBD | B-cells; SARS-CoV2 | 300 | EVQLVESGGGLVKPGGSLRLSCAASGFT |
| 2130 | | | | | | | Human Patient | | FRDVWMSWVRQAPGKGLEWVGRIKS |
| | | | | | | | | | KIDGGTTDYAAPVKGRFTISRDDSKNTLY |
| | | | | | | | | | LQMNSLKTEDTAVYYCTTAGSYYYDTVG |
| | | | | | | | | | PGLPEGKFDYWGQGTLVTVSS |
| COV2- | Ab | SARS-CoV2 | SARS-CoV1 | SARS-CoV2 | | S; RBD | B-cells; SARS-CoV2 | 301 | EVQLVESGGGLIQPGGSLRLSCAASEVT |
| 2132 | | | | | | | Human Patient | | VSSNYMSWVRQAPGKGLEWVSLIYSGG |
| | | | | | | | | | TTYYADSVKGRFTISRDNSKNTLYLQMN |
| | | | | | | | | | SLRAEDTAVYYCARDFLRWHDLWGQG |
| | | | | | | | | | TLVTVSS |
| COV2- | Ab | SARS-CoV2 | SARS-CoV1 | | SARS-CoV1, | S; RBD | B-cells; SARS-CoV2 | 302 | QVQLQESGPGLVKPSETLSLTCTVSGGS |
| 2137 | | | | | SARS-CoV2 | | Human Patient | | VSSGSYYWSWIRQPPGKGLECIGYIYYSG |
| | | | | | | | | | SSNYNPSLKSRVTISVDTSKNQFSLKMSS |
| | | | | | | | | | VTAADTAVYYCAGSPVPPTIVGASYWG |
| | | | | | | | | | QGTLVTVSS |
| COV2- | Ab | SARS-CoV2 | SARS-CoV1 | | SARS-CoV1, | S; non- | B-cells; SARS-CoV2 | 303 | EVQLVQSGAEVKKPGESLKISCKGSGYSF |
| 2142 | | | | | SARS-CoV2 | RBD | Human Patient | | TSYWIDWVRQMPGKGLEWMGIIYPGD |
| | | | | | | | | | SDTRYSPSFQGQVTISADKSTSTAYLQW |
| | | | | | | | | | SSLKASDTAMYYCARRGEAAGIWYFDL |
| | | | | | | | | | WGRGTLVTVSS |
| COV2- | Ab | SARS-CoV2 | SARS-CoV1 | | SARS-CoV1, | S; non- | B-cells; SARS-CoV2 | 304 | EVQLVESGGGLVQPGGSLRLSCAASGFT |
| 2143 | | | | | SARS-CoV2 | RBD | Human Patient | | VSSNYMSWVRQAPGKGLEWVSVIYSA |
| | | | | | | | | | GSTYYADSVKGRFSISRDKSKNTLYLQM |
| | | | | | | | | | NSLRAEDTAVYYCAKEGGSGSLRYYYYG |
| | | | | | | | | | MDVWGQGTTVTVSS |
| COV2- | Ab | SARS-CoV2 | SARS-CoV1 | | SARS-CoV1, | S; NTD | B-cells; SARS-CoV2 | 305 | EVQLVESGGGLVQPGGSLRLSCEASGFT |
| 2146 | | | | | SARS-CoV2 | | Human Patient | | FSSSEINWVRQAPGKGLEWVSHISSSGSI |
| | | | | | | | | | IYYADSVKGRFTISRDNAKNSLYLQMNSL |
| | | | | | | | | | RAEDTAVYYCARRSYRSSWYYYYGMDV |
| | | | | | | | | | WGQGTTVTVSS |
| COV2- | Ab | SARS-CoV2 | SARS-CoV1 | | SARS-CoV1, | S; non- | B-cells; SARS-CoV2 | 306 | QVQLAESGGGVVQPGRSLRLSCAASGF |
| 2147 | | | | | SARS-CoV2 | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARSTSGSYYYGM |
| | | | | | | | | | DVWGQGTTVTVSS |
| COV2- | Ab | SARS-CoV2 | SARS-CoV1 | | SARS-CoV1, | S; NTD | B-cells; SARS-CoV2 | 307 | QVQLVESGGGVVQPGRSLRLSCAASGF |
| 2150 | | | | | SARS-CoV2 | | Human Patient | | TFSTYAMHWVRQAPGKGLEWVAVISY |
| | | | | | | | | | DGSNKYYADSVKGRFTISRDNSKNTLYL |
| | | | | | | | | | QMIGLRAEDTAVYYCARDWAPTYYDM |
| | | | | | | | | | PSAFDIWGQGTMVTVSS |
| COV2- | Ab | SARS-CoV1, | | | SARS-CoV2 | S; non- | B-cells; SARS-CoV2 | 308 | QVQLVQSGAEVRKPGSSVKVSCKASGG |
| 2151 | | SARS-CoV2 | | | | RBD | Human Patient | | TFSSYAISWVRQAPGQGLEWMGGIIPV |
| | | | | | | | | | FGTANYAQKFQGRVTITADKSTSTAFME |
| | | | | | | | | | LNSLRSEDTAVYYCARIGSYPEYFQHWG |
| | | | | | | | | | QGTLVTVSS |
| COV2- | Ab | SARS-CoV1, | | | SARS-CoV2 | S; RBD | B-cells; SARS-CoV2 | 309 | QVQLQESGPGLVKPSQTLSLTCTVSGGSI |
| 2158 | | SARS-CoV2 | | | | | Human Patient | | SSGGYFWSWIRQHPGKGLEWIGSIYYS |
| | | | | | | | | | GSTYYNPSLRSRITISVDTSKNQFSLKLS |
| | | | | | | | | | SVTAADTAVYYCARGGSGSYSLFDYWGQ |
| | | | | | | | | | GTLVTVSS |
| COV2- | Ab | SARS-CoV1, | | | SARS-CoV2 | S; non- | B-cells; SARS-CoV2 | 310 | QVQLAESGGGVVQPGRSLRLSCAASGF |
| 2159 | | SARS-CoV2 | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARSTSGSYYYGM |
| | | | | | | | | | DVWGQGTTVTVSS |
| COV2- | Ab | SARS-CoV1, | | | SARS-CoV2 | S; non- | B-cells; SARS-CoV2 | 311 | QVQLAESGGGVVQPGRSLRLSCAASGF |
| 2160 | | SARS-CoV2 | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARSTSGSYYYGM |
| | | | | | | | | | DVWGQGTTVTVSS |
| COV2- | Ab | SARS-CoV2 | SARS-CoV1 | SARS-CoV2 | | S; RBD | B-cells; SARS-CoV2 | 312 | EVQLVESGGGLVQPGGSLRLSCAASGLT |
| 2165 | | | | | | | Human Patient | | VRSNYMTWVRQTPGKGLEWVSVIYSG |
| | | | | | | | | | GSTFYADSVKGRFTISRDNSKNTVYLQM |
| | | | | | | | | | NSLRAEDTAVYYCARDLVTYGLDVWGQ |
| | | | | | | | | | GTTVTVSS |
| COV2- | Ab | SARS-CoV1, | | | SARS-CoV2 | S; non- | B-cells; SARS-CoV2 | 313 | QVQLVQSGAEVKKPGSSVKVSCKASGG |
| 2166 | | SARS-CoV2 | | | | RBD | Human Patient | | TFSSYAHWVRQAPGQGLEWMGGIIPIF |
| | | | | | | | | | GTTNYAQKFQGRVTITADESTSTAYVELS |
| | | | | | | | | | SLRSEDTAVYYCARIGHFDSSGYYLDYW |
| | | | | | | | | | GQGTLVTVSS |
| COV2- | Ab | SARS-CoV1, | | | SARS-CoV2 | S; non- | B-cells; SARS-CoV2 | 314 | EVQLVESGGGLVQPGGSLRLSCVASGFT |
| 2171 | | SARS-CoV2 | | | | RBD | Human Patient | | FSFYWMSWVRQAPGKGLEWVANIKQ |
| | | | | | | | | | DGGEKYYVDSVKGRFTISRDNAKNSLYL |
| | | | | | | | | | QMNSLRAEDTAVYYCARLSGSSWDFDY |
| | | | | | | | | | WGQGTLVTVSS |
| COV2- | Ab | SARS-CoV2 | SARS-CoV1 | | SARS-CoV1, | S; non- | B-cells; SARS-CoV2 | 315 | EVQLVESGGGVVRPGGSLRLSCAASGFT |
| 2173 | | | | | SARS-CoV2 | RBD | Human Patient | | FDDYGMSWVRQAPGKGLEWVSAINW |
| | | | | | | | | | NGGSTGYADSVKGRFTISRDNAKNSLYL |
| | | | | | | | | | QMNSLRAEDTALYHCARRRSSSRYSSG |
| | | | | | | | | | WYMYYYYMDVWGKGTTVTVSS |
| COV2- | Ab | SARS-CoV1, | | | SARS-CoV2 | S; non- | B-cells; SARS-CoV2 | 316 | EVQLVESGGGLVQPGGSLRLSCVASGFT |
| 2175 | | SARS-CoV2 | | | | RBD | Human Patient | | FSFYWMSWVRQAPGKGLEWVANIKQ |
| | | | | | | | | | DGGEKYYVDSVKGRFTISRDNAKNSLYL |
| | | | | | | | | | QMNSLRAEDTAVYYCARLSGSSWDFDY |
| | | | | | | | | | WGQGTLVTVSS |
| COV2- | Ab | SARS-CoV2 | SARS-CoV1 | | SARS-CoV1, | S; non- | B-cells; SARS-CoV2 | 317 | QITFKESGPTLVKPTETLTLTCTFSGFSV |
| 2177 | | | | | SARS-CoV2 | RBD | Human Patient | | STSGEGVGWIRQPPGKALEWLAVIYWD |
| | | | | | | | | | DDKRYSPSLKSRLTITRDTSKNQVVLTMT |
| | | | | | | | | | NMDPVDTATYYCAHRLWFRDAFDIWG |
| | | | | | | | | | QGTTVTVSS |
| COV2- | Ab | SARS-CoV1, | | | SARS-CoV2 | S; non- | B-cells; SARS-CoV2 | 318 | EVQLVESGGGLVQPGGSLRLSCAASGFT |
| 2178 | | SARS-CoV2 | | | | RBD | Human Patient | | FSTYWMTWVRQAPGKGLEWVANIKQ |
| | | | | | | | | | DGSEKYYVDSVKYRFTISRDNAKNSLYLQ |
| | | | | | | | | | MNSLRAEDTAVYYCARVGSSSWYFDYW |
| | | | | | | | | | GQGTLVTVSS |
| COV2- | Ab | SARS-CoV1, | | | SARS-CoV2 | S; non- | B-cells; SARS-CoV2 | 319 | QVQLVESGGGVVQPGRSLRLSCAASGF |
| 2183 | | SARS-CoV2 | | | | RBD | Human Patient | | TFSSYAMHWVRQAPGKGLEWVAGISY |
| | | | | | | | | | DGSNKYYADSVKGRFTISRDNSKNTLYL |
| | | | | | | | | | QMNSLRAEDTAVYYCARADTMVRGTYF |
| | | | | | | | | | EYWGQGTLVTVSS |
| COV2- | Ab | SARS-CoV1, | | | SARS-CoV2 | S; non- | B-cells; SARS-CoV2 | 320 | QVQLVESGGGVVQPGRSLRLSCAASGF |
| 2187 | | SARS-CoV2 | | | | RBD | Human Patient | | TFSYYPMHWLWVRQAPGKGLEWVAVT |
| | | | | | | | | | SYDGTNKYYADSVKGRFTISRDNSKNTLY |
| | | | | | | | | | LQMNSLRAEDTAVYYCARGGATNFDY |
| | | | | | | | | | WGQGTLVTVSS |
| COV2- | Ab | SARS-CoV1, | | | SARS-CoV2 | S; non- | B-cells; SARS-CoV2 | 321 | EAQLVESGGGLVQPGRSLRLSCAASGFT |
| 2189 | | SARS-CoV2 | | | | RBD | Human Patient | | FDDSAMHWVRQAPGKGLEWVSGISW |
| | | | | | | | | | NSGNVGYADSVKGRFTISRDNAKNSLYL |
| | | | | | | | | | QMNSLRAEDTALYYCTKASRYCSSTICY |
| | | | | | | | | | WNWFDPWGQGTLVTVSS |
| COV2- | Ab | SARS-CoV2 | SARS-CoV1 | | SARS-CoV1, | S; NTD | B-cells; SARS-CoV2 | 322 | EVQLVESGGGLVQPGGSLRLSCAASGFT |
| 2190 | | | | | SARS-CoV2 | | Human Patient | | FSSYEMNWVRQAPGKGLEWVSYISSSG |
| | | | | | | | | | SAIYYADSVKGRFTISRDNAKNSLYLQM |
| | | | | | | | | | NSLRVEDTAVYYCAREARSRYFDWLPSY |
| | | | | | | | | | YFDYWGQGTLVTVSS |
| COV2- | Ab | SARS-CoV2 | SARS-CoV1 | | SARS-CoV1, | S; non- | B-cells; SARS-CoV2 | 323 | EVQLVESGGGVVRPGGSLRLSCAASGFT |
| 2191 | | | | | SARS-CoV2 | RBD | Human Patient | | FDDYGMSWVRQAPGKGLEWVSAINW |
| | | | | | | | | | NGGSTGYADSVKGRFTISRDNAKNSLYL |
| | | | | | | | | | QMNSLRAEDTALYHCARRRSSSRYSSG |
| | | | | | | | | | WYMYYYYMDVWGKGTTVTVSS |
| COV2- | Ab | SARS-CoV2 | SARS-CoV1 | | SARS-CoV1, | S; non- | B-cells; SARS-CoV2 | 324 | QVHLVESGGGVVQPGRSLRLSCAASGF |
| 2195 | | | | | SARS-CoV2 | RBD | Human Patient | | TFSNYGMHWVRQAPGKGLEWVAVISN |
| | | | | | | | | | DEFNKFYANSVKGRFTISRDNSKNTVYL |
| | | | | | | | | | QLNSLRTEDTARYYCAKGGDGSGWAW |
| | | | | | | | | | DGDNPPTDYWGQGTLVIVSS |
| COV2- | Ab | SARS-CoV2 | SARS-CoV1 | SARS-CoV2 | | S; RBD | B-cells; SARS-CoV2 | 325 | QMQLVQSGPEVKKPGTSVKVSCKASGF |
| 2196 | | | | | | | Human Patient | | TFMSSAVQWVRQARGQRLEWIGWIVI |
| | | | | | | | | | GSGNTNYAQKFQERVTITRDMSTSTAY |
| | | | | | | | | | MELSSLRSEDTAVYYCAAPYCSSISCNDG |
| | | | | | | | | | FDIWGQGTMVTVSS |
| COV2- | Ab | SARS-CoV2 | SARS-CoV1 | | SARS-CoV1, | S; non- | B-cells; SARS-CoV2 | 326 | EVQLVQSGAEVKKPGESLKISCKGSGYSF |
| 2197 | | | | | SARS-CoV2 | RBD | Human Patient | | TSYWIGWVRQMPGKGLEWMGIIYPGD |
| | | | | | | | | | SDTRYSPSFQGQVTISADKSISTAYLQWS |
| | | | | | | | | | SLKASDTAMYYCARPDYSSGWFSYWYF |
| | | | | | | | | | DLWGRGTLVTVSS |
| COV2- | Ab | SARS-CoV2 | SARS-CoV1 | | SARS-CoV1, | S; NTD | B-cells; SARS-CoV2 | 327 | QVQLVQSGAEVKKPGASVKVSCKVSGY |
| 2199 | | | | | SARS-CoV2 | | Human Patient | | TLTELSIHWVRQAPGKGLEWMGGFDP |
| | | | | | | | | | EDAETIYAQQFQGRVTMTEDTSTDTAY |
| | | | | | | | | | MELSSLKSEDTALYYCATGFAVFGRAAV |
| | | | | | | | | | PYWGQGTLVTVSS |
| COV2- | Ab | SARS-CoV1, | | | SARS-CoV2 | S; non- | B-cells; SARS-CoV2 | 328 | QVQLVESGGGVVQPGRSLRLSCAASGF |
| 2203 | | SARS-CoV2 | | | | RBD | Human Patient | | TFSTYAMHWVRQAPGKGLAWVALISYD |
| | | | | | | | | | GYNKYYADSVRGRFTISRINSKNTLSLQM |
| | | | | | | | | | NSLRAEDTAVYYCARGSAGNYYYGMDV |
| | | | | | | | | | WGQGTTVTVSS |
| COV2- | Ab | SARS-CoV2 | SARS-CoV1 | | SARS-CoV1, | S; non- | B-cells; SARS-CoV2 | 329 | EVQLVQSGAEVKKPGESLKISCKGSGYSF |
| 2207 | | | | | SARS-CoV2 | RBD | Human Patient | | TSHWIGWVRQMPGKGLEWMGIIYPG |
| | | | | | | | | | DSDTRYSPSFQGQVTISADKSISTAYLQW |
| | | | | | | | | | SSLKASDTAMYYCASALRERGVQLWSV |
| | | | | | | | | | WGQGTLVTVSS |
| COV2- | Ab | SARS-CoV2 | SARS-CoV1 | | SARS-CoV1, | S; NTD | B-cells; SARS-CoV2 | 330 | QVQLVESGGGVVQPGRSLRLSCAASGF |
| 2210 | | | | | SARS-CoV2 | | Human Patient | | TFSSYAMHWVRQAPGKGLEWVAVISY |
| | | | | | | | | | DGSNKYYADSVKGRFTISRDNSKNTLYL |
| | | | | | | | | | QMNSLRVEDTAVYYCARDQEWFRELFL |
| | | | | | | | | | FDYWGQGTLVTVSS |
| COV2- | Ab | SARS-CoV2 | SARS-CoV1 | | SARS-CoV1, | S; NTD | B-cells; SARS-CoV2 | 331 | EVQLVESGGGLVKPGGSLRLSCAASGFT |
| 2212 | | | | | SARS-CoV2 | | Human Patient | | FSSYSMNWVRQAPGKGLEWVSSISNSN |
| | | | | | | | | | SFIYYADSMKGRFTISRDNAKNSLYLQM |
| | | | | | | | | | NSLRAEDTAVYYCARVNGNSNWNFGSY |
| | | | | | | | | | YYYYMDVWGKGTTVTVSS |
| COV2- | Ab | SARS-CoV1, | | | SARS-CoV2 | S; non- | B-cells; SARS-CoV2 | 332 | QVQLVQSGAEVKKPGSSVKVSCKASGG |
| 2214 | | SARS-CoV2 | | | | RBD | Human Patient | | TFSSYAIIWVRQAPGQGLEWMGGIIPIF |
| | | | | | | | | | GTTNYAQKFQGRVTITADESTSTAYVELS |
| | | | | | | | | | SLRSEDTAVYYCARIGHFDSSGYYLDYW |
| | | | | | | | | | GQGTLVTVSS |
| COV2- | Ab | SARS-CoV2 | SARS-CoV1 | | SARS-CoV1, | S; NTD | B-cells; SARS-CoV2 | 333 | EVQLVESGGGLVKPGGSLRLSCAASGFT |
| 2215 | | | | | SARS-CoV2 | | Human Patient | | FSGYSMNWVRQAPGKGLEWVSSISSSS |
| | | | | | | | | | SYIYYADSVKGRFTISRDNAKNSLYLQMN |
| | | | | | | | | | SLRAEDTAVYYCARWLQLRSDYYYFGM |
| | | | | | | | | | DVWGQGTTVTVSS |
| COV2- | Ab | SARS-CoV1, | | | SARS-CoV2 | S; non- | B-cells; SARS-CoV2 | 334 | QVQLVQSGAEVKKPGSSVKVSCKASGG |
| 2216 | | SARS-CoV2 | | | | RBD | Human Patient | | TFSSYAISWVRQAPGQGLEWMGGIIPIF |
| | | | | | | | | | GAANYAQNFQGRVTITADESTSTGYMQ |
| | | | | | | | | | LSSLRFEDTAVYYCARTSHYDSSGSYFEY |
| | | | | | | | | | WGQGTLVTVSS |
| COV2- | Ab | SARS-CoV1, | | | SARS-CoV2 | S; non- | B-cells; SARS-CoV2 | 335 | QVQLVESGGGVVQPGRSLRLSCAASGF |
| 2218 | | SARS-CoV2 | | | | RBD | Human Patient | | TFSSYALFWVRQAPGKGLEWVAVISYD |
| | | | | | | | | | GNNKYYADSVRGRFTISRDNSKNTLYLQ |
| | | | | | | | | | MNSLRPEDTAVYYCARPYTGSYKSYMD |
| | | | | | | | | | VWGKGTTVTVSS |
| COV2- | Ab | SARS-CoV2 | SARS-CoV1 | | SARS-CoV1, | S; NTD | B-cells; SARS-CoV2 | 336 | QVQLQESGPGLVKPSETLSLTCTVSGGSI |
| 2222 | | | | | SARS-CoV2 | | Human Patient | | SSYYWSWIRQPPGKGLEWIGYIYYSGST |
| | | | | | | | | | NYNPSLKSRVTISVDMSKNQFSLKLRSVT |
| | | | | | | | | | AADTAVYYCARAPRERLQWGEYYFDYW |
| | | | | | | | | | GQGTLVTVSS |
| COV2- | Ab | SARS-CoV1, | | | SARS-CoV2 | S; non- | B-cells; SARS-CoV2 | 337 | QVQLVESGGGVVQPGRSLRLSCAASGF |
| 2224 | | SARS-CoV2 | | | | RBD | Human Patient | | TFSTYGMHWVRQAPGKGLEWVAVISY |
| | | | | | | | | | DGSNKYYADSVKGRFTISRDNSKNTLYL |
| | | | | | | | | | QMNSLRAEDTAMYYCAKDGSIAAADY |
| | | | | | | | | | WGQGTLVTVSS |
| COV2- | Ab | SARS-CoV2 | SARS-CoV1 | | SARS-CoV1, | S; non- | B-cells; SARS-CoV2 | 338 | EVQLVQSGAEVKKPGESLKISCKGSGYSF |
| 2226 | | | | | SARS-CoV2 | RBD | Human Patient | | TNSWIGWVRQMPGKGLEWMGIIYPG |
| | | | | | | | | | DSDTRYSPSFQGQVTISADKSISTAYLQW |
| | | | | | | | | | SSLKASDTAIYYCATHRCSGGFCYLAYWG |
| | | | | | | | | | QGTLVTVSS |
| COV2- | Ab | SARS-CoV1, | | | SARS-CoV2 | S; non- | B-cells; SARS-CoV2 | 339 | QVQLVESGGGVVQPGRSLRLSCAASGF |
| 2227 | | SARS-CoV2 | | | | RBD | Human Patient | | TFSSYGMHWVRQAPGKGLEWVAVIWY |
| | | | | | | | | | DGSKKDYADSVKGRFTISRDNSKNTLYL |
| | | | | | | | | | QMNSLRAEDTAVYYCARDQSQGAYILT |
| | | | | | | | | | GYRGYGMDVWGQGTTVTVSS |
| COV2- | Ab | SARS-CoV2 | SARS-CoV1 | | SARS-CoV1, | S; NTD | B-cells; SARS-CoV2 | 340 | QVQLQQWGAGLLKPSETLSLTCAVYGG |
| 2228 | | | | | SARS-CoV2 | | Human Patient | | SFSGHYWSWIRQPPGKGLEWIGEINHS |
| | | | | | | | | | GSTNYNPSLKSRVTISVDTSKNQFSLKLSS |
| | | | | | | | | | VTAADTAVYYCARPPQAARIHYYYYMD |
| | | | | | | | | | VWGKGTTVTVSS |
| COV2- | Ab | SARS-CoV1, | | | SARS-CoV2 | S; RBD | B-cells; SARS-CoV2 | 341 | EVQLVESGGGLVQPGRSLRLSCAASGFT |
| 2231 | | SARS-CoV2 | | | | | Human Patient | | FDDYAMNWVRQPPGKGLEWVSGISW |
| | | | | | | | | | NSDSIGYADSVKGRFTISRDNAKNSLYLQ |
| | | | | | | | | | MNSLRAEDTAMYYCAKGRGAGYTSYM |
| | | | | | | | | | DVWGKGTTVTVSS |
| COV2- | Ab | SARS-CoV1, | | | SARS-CoV2 | S; non- | B-cells; SARS-CoV2 | 342 | EVQLVESGGGLIQPGGSLRLSCAASGFIV |
| 2235 | | SARS-CoV2 | | | | RBD | Human Patient | | SSNYMSWVRQAPGKGLEWVSVIYSGG |
| | | | | | | | | | STYYADSVKGRFTISRDNSKNTLYLQMN |
| | | | | | | | | | SLRAEDTAVYYCARESTQWGQGTLVTV |
| | | | | | | | | | SS |
| COV2- | Ab | SARS-CoV2 | SARS-CoV1 | | SARS-CoV1, | S; non- | B-cells; SARS-CoV2 | 343 | QITLKESGPPLVEPKQTLTLTCTFSGFSL |
| 2238 | | | | | SARS-CoV2 | RBD | Human Patient | | TTSGEAVGWIRQPPGKALEWLALIYWDD |
| | | | | | | | | | DKHYSPSLRNRLTITRDTSKNQVVLTLTN |
| | | | | | | | | | VDPADTGTYYCAHRAVILNFDHWGQGF |
| | | | | | | | | | LVTVSS |
| COV2- | Ab | SARS-CoV1, | | | SARS-CoV2 | S; non- | B-cells; SARS-CoV2 | 344 | QVQLVESGGGVVQPGRSLRLSCAASGF |
| 2239 | | SARS-CoV2 | | | | RBD | Human Patient | | TFSSYAMHWVRQAPGKGLEWVAVISY |
| | | | | | | | | | DGINKYYADAVKGRFTISRDNSKNTLYL |
| | | | | | | | | | QMNSLRAEDTAVYYCARPRSGSYYAYFD |
| | | | | | | | | | YWGQGTLVTVSS |
| COV2- | Ab | SARS-CoV2 | SARS-CoV1 | | SARS-CoV1, | S; RBD | B-cells; SARS-CoV2 | 345 | EVQLVESGGGLVQPGRSLRLSCAASGFT |
| 2240 | | | | | SARS-CoV2 | | Human Patient | | FDDYAMHWVRQAPGKGLEWVSGISW |
| | | | | | | | | | NSGSIGYADSVKGRFTISRDNAKNSLYLQ |
| | | | | | | | | | MNSLRAEDTALYYCAKVGYTISRQWLV |
| | | | | | | | | | GEFDYWGQGTLVTVSS |
| COV2- | Ab | SARS-CoV2 | SARS-CoV1 | | SARS-CoV1, | S; non- | B-cells; SARS-CoV2 | 346 | EVQLVQSGAEVKKPGESLKISCKGSGYSF |
| 2241 | | | | | SARS-CoV2 | RBD | Human Patient | | TSYWIDWVRQMPGKGLEWMGIIYPGD |
| | | | | | | | | | SDTRYSPSFQGQVTISADKSTSTAYLQW |
| | | | | | | | | | SSLKASDTAMYYCARRGEAAGIWYFDL |
| | | | | | | | | | WGRGTLVTVSS |
| COV2- | Ab | SARS-CoV2 | SARS-CoV1 | | SARS-CoV1, | S; RBD | B-cells; SARS-CoV2 | 347 | QLQLQESGPGLVKPSETLPLTCTVSGGSI |
| 2243 | | | | | SARS-CoV2 | | Human Patient | | SSSSYYWGWIRQPPGKGLQWIGNIYYS |
| | | | | | | | | | GSTYYNSSLKSRVTISVDTSKNQFSLKLS |
| | | | | | | | | | SVTAADTAVYYCARQSRGYSYAWSFDYW |
| | | | | | | | | | GQGTLVTVSS |
| COV2- | Ab | SARS-CoV2 | SARS-CoV1 | | SARS-CoV1, | S; non- | B-cells; SARS-CoV2 | 348 | QVQLVESGGGVVQPGRSLRLSCAASGF |
| 2245 | | | | | SARS-CoV2 | RBD | Human Patient | | TFSNYGIHWVRQAPGQGLEWVAGIWY |
| | | | | | | | | | DGSNKYYVDSVKGRFTISRDNSKNTLYL |
| | | | | | | | | | QMNSLRAEDTAVYYCAGSSGEGGLYYY |
| | | | | | | | | | YGMDVWGQGTTVTVSS |
| COV2- | Ab | SARS-CoV2 | SARS-CoV1 | | SARS-CoV1, | S; non- | B-cells; SARS-CoV2 | 349 | QVQLVESGGGVVQPGRSLRLSCAASGF |
| 2248 | | | | | SARS-CoV2 | RBD | Human Patient | | TFDSYGVHWVRQAPGKGLEWVAVISYD |
| | | | | | | | | | GSNKHYADSVKGRFTISRDNSKNTLYVQ |
| | | | | | | | | | MNSLRTEDTAVYYCARDSGGNYGDSYF |
| | | | | | | | | | DYWGQGTLVTVSS |
| COV2- | Ab | SARS-CoV1, | | | SARS-CoV2 | S; non- | B-cells; SARS-CoV2 | 350 | QVQLVESGGGVVQPGRSLRLSCAASGF |
| 2250 | | SARS-CoV2 | | | | RBD | Human Patient | | TFSTYAMHWVRQAPGKGLAWVALISYD |
| | | | | | | | | | GYNKYYADSVRGRFTISRINSKNTLSLQM |
| | | | | | | | | | NSLRAEDTAVYYCARGSAGNYYYGMDV |
| | | | | | | | | | WGQGTTVTVSS |
| COV2- | Ab | SARS-CoV2 | SARS-CoV1 | | SARS-CoV1, | S; NTD | B-cells; SARS-CoV2 | 351 | QVTLKESGPVLVKPTETLTLTCAVSGFSL |
| 2251 | | | | | SARS-CoV2 | | Human Patient | | SNAKMGVSWIRQPPGKALEWLAHIFSN |
| | | | | | | | | | DEKAYSTSLKTRLTISKDTSKSQVVLTVT |
| | | | | | | | | | NMDPVDTATYYCARIVLGASGTYPSPGFD |
| | | | | | | | | | PWGQGTLVTVSS |
| COV2- | Ab | SARS-CoV2 | SARS-CoV1 | | SARS-CoV1, | S; RBD | B-cells; SARS-CoV2 | 352 | EVQLVESGGGLVQPGGSLRLSCAASGFT |
| 2253 | | | | | SARS-CoV2 | | Human Patient | | FSSYWMHWVRQVPGKGLVWVSRINSD |
| | | | | | | | | | GSSTSYADSVKGRFTISRDNAKNTLYLE |
| | | | | | | | | | MNSLRAQDTAVYYCAGSPWLRGDIDY |
| | | | | | | | | | WGQGTLVTVSS |
| COV2- | Ab | SARS-CoV1, | | | SARS-CoV2 | S; RBD | B-cells; SARS-CoV2 | 353 | QVQLVQSGSELTKPGASVKVSCKASGYT |
| 2256 | | SARS-CoV2 | | | | | Human Patient | | FTSYAMNWVRQAPGQGLEWMGWINT |
| | | | | | | | | | DTGNPTYAQGFTGRFVFSLDTSVSTAYL |
| | | | | | | | | | QISSLKAEDTAVYYCARDPSYCSSTRCYT |
| | | | | | | | | | VGWFDPWGQGTLVTVSS |
| COV2- | Ab | SARS-CoV2 | SARS-CoV1 | | SARS-CoV1, | S; NTD | B-cells; SARS-CoV2 | 354 | QVQLQESGPGLVKPSQTLSLTCTVSGDSI |
| 2257 | | | | | SARS-CoV2 | | Human Patient | | NSGNYYWSWIRQPAGKGLEWIGRMFT |
| | | | | | | | | | SGSTNYNPSLTSRVTMSIDTSKNQFSLNL |
| | | | | | | | | | NSVTAADTAMYYCARGHVAAWESCYY |
| | | | | | | | | | WGQGILVTVSS |
| COV2- | Ab | SARS-CoV1, | | SARS-CoV2 | | S; RBD | B-cells; SARS-CoV2 | 355 | EVQLVESGGGVVRPGGSLRLSCAASGFT |
| 2258 | | SARS-CoV2 | | | | | Human Patient | | LDDYGLSWVRHAPGKGLEWVSGINWN |
| | | | | | | | | | GGRTAYADSVKGRFTISRDNAKNSLYLQ |
| | | | | | | | | | MNSLRAEDTALYHCARARGPSEQYYDLL |
| | | | | | | | | | TGYYDAFDIWGQGTMVTVSS |
| COV2- | Ab | SARS-CoV2 | SARS-CoV1 | | SARS-CoV1, | S; NTD | B-cells; SARS-CoV2 | 356 | EVQLLESGGGLVHPGGSLRLSCAASGFT |
| 2260 | | | | | SARS-CoV2 | | Human Patient | | FSSYALSWVRQAPGKGLEWVSAISGSG |
| | | | | | | | | | GSTYYADSVKGRFTISRDNSKNTLYLQM |
| | | | | | | | | | NRLRAEDTAVYYCAQMGPLGSTSSAAD |
| | | | | | | | | | YWGQGTLVTVSS |
| COV2- | Ab | SARS-CoV2 | SARS-CoV1 | SARS-CoV2 | | S; non- | B-cells; SARS-CoV2 | 357 | QVQLVQSGAEVKKPGASVKVSCKASGY |
| 2262 | | | | (weak) | | RBD | Human Patient | | TFTSYDINWVRQATGQGLEWMGWMN |
| | | | | | | | | | PNSGNTGYAQKFQGRVTMTRNTSISTA |
| | | | | | | | | | YMELSSLRSEDTALYYCAREARYFDWIFE |
| | | | | | | | | | GSDYYYYGMDVWGQGTTVTVSS |
| COV2- | Ab | SARS-CoV2 | SARS-CoV1 | | SARS-CoV1, | S; NTD | B-cells; SARS-CoV2 | 358 | EVQLVESGGGLVQPGGSLRLSCEASGFT |
| 2263 | | | | | SARS-CoV2 | | Human Patient | | FSSSEINWVRQAPGKGLEWVSHISSSGSI |
| | | | | | | | | | IYYADSVKGRFTISRDNAKNSLYLQMNSL |
| | | | | | | | | | RAEDTAVYYCARRSYRSSWYYYYGMDV |
| | | | | | | | | | WGQGTTVTVSS |
| COV2- | Ab | SARS-CoV1, | | | SARS-CoV2 | S; non- | B-cells; SARS-CoV2 | 359 | EVQLVESGGGLVQPGRSLRLSCTASGFIF |
| 2266 | | SARS-CoV2 | | | | RBD | Human Patient | | GDYAMGWVRQAPGKGLEWVGFIRGK |
| | | | | | | | | | AYDGTTEYAASVKGRFTISRDDSKYIAHL |
| | | | | | | | | | QMNSLKTEDTAVYYCIRDYDFWGGYYY |
| | | | | | | | | | HPLRAFDIWGRGTMVTVSS |
| COV2- | Ab | SARS-CoV2 | SARS-CoV1 | SARS-CoV2 | | S; RBD | B-cells; SARS-CoV2 | 360 | QITLKESGPTLVKPTQTLTLTCTFSGFSL |
| 2268 | | | | | | | Human Patient | | STSGVGVGWIRQPPGKALEWLALIYWD |
| | | | | | | | | | DDKRYSPSLKSRLSITKDTSKNQVVLTMT |
| | | | | | | | | | NMDPVDTGTYYCARHQIVVLFDMWG |
| | | | | | | | | | QGTRVTVSS |
| COV2- | Ab | SARS-CoV1, | | | SARS-CoV2 | S; non- | B-cells; SARS-CoV2 | 361 | QVQLVQSGAEVKKPGSSVKVSCKASGG |
| 2270 | | SARS-CoV2 | | | | RBD | Human Patient | | TFSSYAISWVRQAPGQGLEWMGGIIPIF |
| | | | | | | | | | GTANYAQKFQGRVTITADESTSTAYMEL |
| | | | | | | | | | SSLRSEDTAVYYCAITYYYDSSGYWWDD |
| | | | | | | | | | WGQGTLVTVSS |
| COV2- | Ab | SARS-CoV1, | | | SARS-CoV2 | S; non- | B-cells; SARS-CoV2 | 362 | QLQLQESGPGLVKPSETLSLTCTVSGGSI |
| 2273 | | SARS-CoV2 | | | | RBD | Human Patient | | SSSSYYWGWIRQPPGKGLEWIGTIYYSG |
| | | | | | | | | | STYYNPSLKSRVTISVDTSKKQFSLKLSS |
| | | | | | | | | | VTAADTAVYYCAGEEVRGVKLYYYYAMD |
| | | | | | | | | | VWGQGTTVTVSS |
| COV2- | Ab | SARS-CoV1, | | | SARS-CoV2 | S; non- | B-cells; SARS-CoV2 | 363 | EVQLVESGGRLVLPGGSLRLSCAASGFTF |
| 2274 | | SARS-CoV2 | | | | RBD | Human Patient | | SVYEMNWVRQAPGKGLEWLSYIGTSGS |
| | | | | | | | | | PIYYADSVKGRFTVSRDNAKNSLYLQMN |
| | | | | | | | | | SLRVEDTALYYCARDRGWNYGLDYWG |
| | | | | | | | | | QGTLVTVSS |
| COV2- | Ab | SARS-CoV2 | SARS-CoV1 | | SARS-CoV1, | S; RBD | B-cells; SARS-CoV2 | 364 | QVQLVESGGGVVQPGRSLRLSCAASGF |
| 2277 | | | | | SARS-CoV2 | | Human Patient | | TFSSYAMHWVRQAPGKGLEWVALISSD |
| | | | | | | | | | GGNKFYADSVKGRFTISRDNSKNTLYLQ |
| | | | | | | | | | MNSLRAEDTAVYYCARDVPTTVTAFTVF |
| | | | | | | | | | TYWGQGTLVTVSS |
| COV2- | Ab | SARS-CoV1, | | | SARS-CoV2 | S; non- | B-cells; SARS-CoV2 | 365 | QVQLVESGGGVVQPGRSLRLSCAASGF |
| 2281 | | SARS-CoV2 | | | | RBD | Human Patient | | AFSNYAIHWVRQAPGKGLEWVAVISYD |
| | | | | | | | | | GNNKDYADSVKGRFTISRDNSKNTLYLQ |
| | | | | | | | | | MNSLRAEDTAVYYCARVPVMVRGVYF |
| | | | | | | | | | DYWGQGTLVTVSS |
| COV2- | Ab | SARS-CoV1, | | | SARS-CoV2 | S; RBD | B-cells; SARS-CoV2 | 366 | QVQLVQSGAEVKKPGASVKVSCKVSGY |
| 2287 | | SARS-CoV2 | | | | | Human Patient | | TFTSYDINWVRQATGQGLEWMGWMN |
| | | | | | | | | | PNSGHTGYAQKFQGRVTMTRNTSISTA |
| | | | | | | | | | YMELSSLRYEDTAVYYCARGYGLTYYMD |
| | | | | | | | | | VWGKGTTVTVSS |
| COV2- | Ab | SARS-CoV2 | SARS-CoV1 | SARS-CoV2 | | S; RBD | B-cells; SARS-CoV2 | 367 | QVQLAQSGAEVKKPGASVKVSCKAAGY |
| 2290 | | | | (weak) | | | Human Patient | | TFTSYDINWVRQATGQGLEWMGWMN |
| | | | | | | | | | PNSGNAGYAQKFQGRVTMTRDTSISTA |
| | | | | | | | | | YMELSSLRSEDTAVYYCARMRSGWPTH |
| | | | | | | | | | GRPDDFWGRGTLVTVSS |
| COV2- | Ab | SARS-CoV2 | SARS-CoV1 | | SARS-CoV1, | S; NTD | B-cells; SARS-CoV2 | 368 | EVQLLQSGGGLVQPGGSLRLSCAASGFT |
| 2293 | | | | | SARS-CoV2 | | Human Patient | | FRNYAMSWVRQAPGKGLEWVSAISGS |
| | | | | | | | | | GGTTYYADSVKGRFTISRDNSKNTLYLQ |
| | | | | | | | | | MNSLRAEDTAVYYCAKNERITMLVVVTL |
| | | | | | | | | | FDYWGQGTLVTVSS |
| COV2- | Ab | SARS-CoV2 | SARS-CoV1 | | SARS-CoV1, | S; non- | B-cells; SARS-CoV2 | 369 | EVQLVESGGGLVQPGGSLRLSCAASGFT |
| 2296 | | | | | SARS-CoV2 | RBD | Human Patient | | VSGNYMSWVRQAPGKGLEWVSVIYSG |
| | | | | | | | | | GSTYYADSVKGRFTISRDNSKNTLYLQM |
| | | | | | | | | | NSPRAEDTAVYYCARDPSAYYDILTGYSG |
| | | | | | | | | | DVWGKGTTVTVSS |
| COV2- | Ab | SARS-CoV2 | SARS-CoV1 | | SARS-CoV1, | S; RBD | B-cells; SARS-CoV2 | 370 | QVQLVESGGGVVQPGRSLRLSCAASGF |
| 2299 | | | | | SARS-CoV2 | | Human Patient | | SFSSYVMNWVRQAPGKGLEWVAVISY |
| | | | | | | | | | DGSSKYYADSVKGRFTISRDNSKNTLYLQ |
| | | | | | | | | | MNSLRAEDTAVYYCARDIDSGYDPTPVF |
| | | | | | | | | | DYWGQGTLVTVSS |
| COV2- | Ab | SARS-CoV2 | SARS-CoV1 | | SARS-CoV1, | S; non- | B-cells; SARS-CoV2 | 371 | QVQLQESGPGLVKPSGTLSLTCAVSGGS |
| 2300 | | | | | SARS-CoV2 | RBD | Human Patient | | ISSSNWWTWVRQPPGKGLEWIGEIYHS |
| | | | | | | | | | GSTNYNPSLKSRVTISVDKSKNQFSLKLS |
| | | | | | | | | | SVTAADTAVYYCASRWGDYFDSSGAYDS |
| | | | | | | | | | WGQGTLVTVSS |
| COV2- | Ab | SARS-CoV2 | SARS-CoV1 | | SARS-CoV1, | S; RBD | B-cells; SARS-CoV2 | 372 | EVQLVESGGGLVKPGGSLRLSCAASGFT |
| 2304 | | | | | SARS-CoV2 | | Human Patient | | FSSYSMNWVRQAPGKGLEWVSCISSSS |
| | | | | | | | | | SFIYYADSVKGRFTISRDNAKNSLYLQMN |
| | | | | | | | | | SLRAEDTAVYYCARDPVWVDGELLSGGI |
| | | | | | | | | | PFDYWGQGTLVTVSS |
| COV2- | Ab | SARS-CoV2 | SARS-CoV1 | | SARS-CoV1, | S; NTD | B-cells; SARS-CoV2 | 373 | QLQLQESGPGLVKPSETLSLTCTVSGGSI |
| 2305 | | | | | SARS-CoV2 | | Human Patient | | RSSSYYWGWIRQPPGKGLEWIASIYYSG |
| | | | | | | | | | STYYNSSLKSRVTISVDTSKNQFSLKVNS |
| | | | | | | | | | MTAADTAVYYCAILWRGSSWADRHYYY |
| | | | | | | | | | YSMDVWGQGTTVTVSS |
| COV2- | Ab | SARS-CoV2 | SARS-CoV1 | | SARS-CoV1, | S; NTD | B-cells; SARS-CoV2 | 374 | QVQLVESGGGVVQPGRSLRLSCTASGFT |
| 2307 | | | | | SARS-CoV2 | | Human Patient | | FSSYAMHWVRQAPGKGLEWVALISYD |
| | | | | | | | | | GNNKYYADSAKGRFTISRDNSKNTLYLQ |
| | | | | | | | | | MNSLRSEDTAVYYCARDLGRGLDPWG |
| | | | | | | | | | QGTLVTVSS |
| COV2- | Ab | SARS-CoV2 | SARS-CoV1 | SARS-CoV2 | | S; RBD | B-cells; SARS-CoV2 | 375 | EVQLLESGGGLIQPGGSLRLSCAASGFTF |
| 2308 | | | | | | | Human Patient | | SNYAMSWVRQAPGKGLEWVSGIISSSG |
| | | | | | | | | | GATYNADSVRGRFTTSRDNSKNILYLQM |
| | | | | | | | | | NSLRGEDTAVYYCVKGLFDWFPLWGQ |
| | | | | | | | | | GTMVTVSS |
| COV2- | Ab | SARS-CoV1, | | | SARS-CoV2 | S; non- | B-cells; SARS-CoV2 | 376 | QVQLQESGPGLVKPSQTLSLTCAVSGAS |
| 2310 | | SARS-CoV2 | | | | RBD | Human Patient | | ISSGSYYWSWIRQPAGKGLEWIGRIYTS |
| | | | | | | | | | GNTNYNPSLKSRVTISVDTSKNQFSLKLS |
| | | | | | | | | | SVTAADTAVYYCATGYIGTYYYYMDVW |
| | | | | | | | | | GKGTTVTVSS |
| COV2- | Ab | SARS-CoV1, | | | SARS-CoV2 | S; RBD | B-cells; SARS-CoV2 | 377 | EVQLVESGGGLVQPGRSLRLSCAASGFT |
| 2313 | | SARS-CoV2 | | | | | Human Patient | | FDDYAMHWVRQAPGKGLEWVSGISW |
| | | | | | | | | | NSGSIGYADSVKGRFTISRDNAKNSLYLQ |
| | | | | | | | | | MNSLRAEDTALYYCAKVSSITSLLGYYFD |
| | | | | | | | | | SWGQGTLVTVSS |
| COV2- | Ab | SARS-CoV2 | SARS-CoV1 | | SARS-CoV1, | S; RBD | B-cells; SARS-CoV2 | 378 | QVTLRESGPALVKPTQTLSLTCTFSGFSL |
| 2318 | | | | | SARS-CoV2 | | Human Patient | | GTSGMCVSWIRQPPGKALEWLARIDW |
| | | | | | | | | | DDDKYYSTSLKTRLTISKDTSKNQVVLTM |
| | | | | | | | | | TNMDPVDTATYYCARGVVTYDYWGQG |
| | | | | | | | | | TLVTVSS |
| COV2- | Ab | SARS-CoV1, | | | SARS-CoV2 | S; non- | B-cells; SARS-CoV2 | 379 | QVQLVESGGGVVQPGRSLRLSCAASGF |
| 2322 | | SARS-CoV2 | | | | RBD | Human Patient | | TFSNYAMHWVRQAPGKGLDWVAVISY |
| | | | | | | | | | DGSNRYYAASVKGRFTISRDNSKNTLYL |
| | | | | | | | | | QMNSLRTEDTAVYFCARGDGYRSQFDP |
| | | | | | | | | | WGQGTLVTVSS |
| COV2- | Ab | SARS-CoV1, | | | SARS-CoV2 | S; non- | B-cells; SARS-CoV2 | 380 | QITFKESGPTLVKPTETLTLTCTFSGFSV |
| 2325 | | SARS-CoV2 | | | | RBD | Human Patient | | STSGEGVGWIRQPPGKALEWLAVIYWD |
| | | | | | | | | | DDKRYSPSLKSRLTITRDTSKNQVVLTMT |
| | | | | | | | | | NMDPVDTATYYCAHRLWFRDAFDIWG |
| | | | | | | | | | QGTTVTVSS |
| COV2- | Ab | SARS-CoV1, | | | SARS-CoV2 | S; RBD | B-cells; SARS-CoV2 | 381 | EVQLLESGGGLVQPGGSLRLSCAASGFT |
| 2329 | | SARS-CoV2 | | | | | Human Patient | | FNNYAMSWVRQAPGKGLEWVSAIGGS |
| | | | | | | | | | GGSTYYADSVKGRFTVSRDNSENTLYLQ |
| | | | | | | | | | MSSLRAEDTAVYYCARVEGDWLLGGPY |
| | | | | | | | | | YHYYGMDVWGQGTTVTVSS |
| COV2- | Ab | SARS-CoV1, | | | SARS-CoV2 | S; RBD | B-cells; SARS-CoV2 | 382 | QLQLQESGPGLVKPSETLSLTCTVSGGSI |
| 2331 | | SARS-CoV2 | | | | | Human Patient | | SSSSYYWGWIRQPPGNGLEWIGSIYYSG |
| | | | | | | | | | STYYNPSLKGRVSISVDTSKNQFSLKLSS |
| | | | | | | | | | VTAADTAVYYCARILVIFTLNWFDPWGQ |
| | | | | | | | | | GTLVTVSS |
| COV2- | Ab | SARS-CoV1, | | | SARS-CoV2 | S; non- | B-cells; SARS-CoV2 | 383 | EVQLVESGGGLVQPGGSLRLSCAASGFT |
| 2333 | | SARS-CoV2 | | | | RBD | Human Patient | | FSIYWMSWVRQAPGKGLQWVANIKQ |
| | | | | | | | | | DASEKYYVDSVKGRFTISRDNAKNSLYLQ |
| | | | | | | | | | MNSLRAEDTAVYYCARLGGSSWHFDY |
| | | | | | | | | | WGQGTLVTVSS |
| COV2- | Ab | SARS-CoV2 | SARS-CoV1 | | SARS-CoV1, | S; NTD | B-cells; SARS-CoV2 | 384 | QLQLQESGPGLVKPSETLSLTCTVSGGPI |
| 2335 | | | | | SARS-CoV2 | | Human Patient | | SSSRYYWGWIRQPPGKGLEWIGSIYYSG |
| | | | | | | | | | STYYNPSLKSRVTISVDTSKNQFSLKLNS |
| | | | | | | | | | VTAADTAVYYCARHDGSGEMDTITWGPI |
| | | | | | | | | | YYYMDVWGKGTTVTVSS |
| COV2- | Ab | SARS-CoV1, | | | SARS-CoV2 | S; non- | B-cells; SARS-CoV2 | 385 | QITFKESGPTLVKPTETLTLTCTFSGFSV |
| 2337 | | SARS-CoV2 | | | | RBD | Human Patient | | STSGEGVGWIRQPPGKALEWLAVIYWD |
| | | | | | | | | | DDKRYSPSLKSRLTITRDTSKNQVVLTMT |
| | | | | | | | | | NMDPVDTATYYCAHRLWFRDAFDIWG |
| | | | | | | | | | QGTTVTVSS |
| COV2- | Ab | SARS-CoV1, | | | SARS-CoV2 | S; non- | B-cells; SARS-CoV2 | 386 | QVQLVQSGAEVKKPGASVKVSCKASGY |
| 2340 | | SARS-CoV2 | | | | RBD | Human Patient | | TFTSYGMHWVRQAPGQRLEWMGWIN |
| | | | | | | | | | VGNGNTKYSQRFQGRVTITRDTSASTAY |
| | | | | | | | | | MELSSLRSEDTAVYYCAMGPSAFSWLD |
| | | | | | | | | | PWGQGTLVTVSS |
| COV2- | Ab | SARS-CoV1, | | | SARS-CoV2 | S; non- | B-cells; SARS-CoV2 | 387 | QVQLAESGGGVVQPGRSLRLSCAASGF |
| 2341 | | SARS-CoV2 | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARSTSGSYYYGM |
| | | | | | | | | | DVWGQGTTVTVSS |
| COV2- | Ab | SARS-CoV1, | | | SARS-CoV2 | S; RBD | B-cells; SARS-CoV2 | 388 | QITLKESGPTLVKPTQTLTLTCTFSGFSL |
| 2342 | | SARS-CoV2 | | | | | Human Patient | | NTSGVGVGWIRQPPGKALEWLALIYWD |
| | | | | | | | | | DDKRYNPSLKSRLTITKDTSKNQVVLTM |
| | | | | | | | | | TNMDPVDTATYYCAHRPPSYHGWCYF |
| | | | | | | | | | DYWGQGNLVTVSS |
| COV2- | Ab | SARS-CoV1, | | | SARS-CoV2 | S; non- | B-cells; SARS-CoV2 | 389 | QVQVVQSGAEVKKPGASVKVSCKASGY |
| 2343 | | SARS-CoV2 | | | | RBD | Human Patient | | TFKNYGISWVRQAPGQGLEWMGWISA |
| | | | | | | | | | YTGNTNYAQKFQGRMTMTTDTSTGTG |
| | | | | | | | | | YMELRSLRSDDTAVYYCARVQRRRLDY |
| | | | | | | | | | WGQGTLVIVSS |
| COV2- | Ab | SARS-CoV2 | SARS-CoV1 | | SARS-CoV1, | S; NTD | B-cells; SARS-CoV2 | 390 | QVQLQQWGAGLLKPSETLSLTCAVYGG |
| 2346 | | | | | SARS-CoV2 | | Human Patient | | SFSGHYWSWIRQPPGKGLEWIGEINHS |
| | | | | | | | | | GSTNYNPSLKSRVTISVDTSKNQFSLKLS |
| | | | | | | | | | SVTAADTAVYYCARPPQAARIHYYYYMD |
| | | | | | | | | | VWGKGTTVTVSS |
| COV2- | Ab | SARS-CoV2 | SARS-CoV1 | | SARS-CoV1, | S; RBD | B-cells; SARS-CoV2 | 391 | EVQLVQSGAEVKKPGESLKISCKGSGYSF |
| 2351 | | | | | SARS-CoV2 | | Human Patient | | TSYWIGWVRQMPGKGLEWMGIIYPGD |
| | | | | | | | | | SDTTYNPSFQGQVTISADKSLTTAFLHW |
| | | | | | | | | | SSLKASDTAIYYCARRFYGPSSFDYWGQ |
| | | | | | | | | | GTLVIVSS |
| COV2- | Ab | SARS-CoV2 | SARS-CoV1 | | SARS-CoV1, | S; RBD | B-cells; SARS-CoV2 | 392 | QVQLVQSGAEVKRPGASMNISCKASGY |
| 2352 | | | | | SARS-CoV2 | | Human Patient | | NFNNNYIYWVRQAPGQGLEWMGVVN |
| | | | | | | | | | PTGGGTAYAQGFQDRVTITSDTPRNTVY |
| | | | | | | | | | LGVTGLHSEDTAVYFCARGGEWRIVPG |
| | | | | | | | | | GRDYFDYWGQGTLVTVSA |
| COV2- | Ab | SARS-CoV2 | SARS-CoV1 | SARS-CoV2 | | S; RBD | B-cells; SARS-CoV2 | 393 | EVQLVQSGAEVKKPGESLKISCKGFGYN |
| 2353 | | | | | | | Human Patient | | FTNYWIGWVRQMPGKGLEWMGIIYPG |
| | | | | | | | | | DSETRNSPSFQGQVTISADKSMSTAYLQ |
| | | | | | | | | | WSSLKASDTAMYYCARLGVSKYCSGGR |
| | | | | | | | | | CLSGGSNWFDPWGQGTLVTVSS |
| COV2- | Ab | SARS-CoV1, | | SARS-CoV2 | | S; RBD | B-cells; SARS-CoV2 | 394 | EVHLVGSGGGLIQPGGSLRLSCAASGFT |
| 2354 | | SARS-CoV2 | | | | | Human Patient | | VSSNFMSWVRQAPGKGLEWVSIIHNG |
| | | | | | | | | | GDSYYTDSVKGRFTISRDNSKNTLYLQIV |
| | | | | | | | | | INNLRAEDTAVYYCASSSWLRGAFDIWG |
| | | | | | | | | | QGTMVTVSS |
| COV2- | Ab | SARS-CoV2 | SARS-CoV1 | SARS-CoV2 | | S; RBD | B-cells; SARS-CoV2 | 395 | QMQLVQSGPEVKKPGTSVKVSCKTSGF |
| 2355 | | | | | | | Human Patient | | TFTSSAIQWVRQARGQRLEWIGWIVVG |
| | | | | | | | | | SGNTNYAQKFQERVTITRDMSTSTAYM |
| | | | | | | | | | ELSSLRSEDTAVYYCAAPHCNRTSCYDAF |
| | | | | | | | | | DLWGQGTMVTVSS |
| COV2- | Ab | SARS-CoV1, | | | SARS-CoV2 | S; RBD | B-cells; SARS-CoV2 | 396 | EVQLVESGGDLVKPGRSLRLSCSASGFTF |
| 2357 | | SARS-CoV2 | | | | | Human Patient | | GDYTMSWFRQAPGKGLEWVAFIRSKAY |
| | | | | | | | | | GGTTEYAASVIGRFTISRDDSKSIAYLQM |
| | | | | | | | | | NSLKSEDTAVYYCSRVRGSFYGSVGKNY |
| | | | | | | | | | GMDVWGQGTTVTVSS |
| COV2- | Ab | SARS-CoV1, | | | SARS-CoV2 | S; RBD | B-cells; SARS-CoV2 | 397 | QVQLVESGGGVVQPGRSLRLSCAASGF |
| 2358 | | SARS-CoV2 | | | | | Human Patient | | SFSSYVMNWVRQAPGKGLEWVAVISY |
| | | | | | | | | | DGSSKYYADSVKGRFTISRDNSKNTLYLQ |
| | | | | | | | | | MNSLRAEDTAVYYCARDIDSGYDPTPVF |
| | | | | | | | | | DYWGQGTLVTVSS |
| COV2- | Ab | SARS-CoV1, | | | SARS-CoV2 | S; non- | B-cells; SARS-CoV2 | 398 | QVQLVQSGAEVKKPGSSVKVSCKASGG |
| 2367 | | SARS-CoV2 | | | | RBD | Human Patient | | TFSSYAISWVRQAPGQGLEWMGGIIPIF |
| | | | | | | | | | GAANYAQNFQGRVTITADESTSTGYMQ |
| | | | | | | | | | LSSLRFEDTAVYYCARTSHYDSSGSYFEY |
| | | | | | | | | | WGQGTLVTVSS |
| COV2- | Ab | SARS-CoV2 | SARS-CoV1 | | SARS-CoV1, | S; non- | B-cells; SARS-CoV2 | 399 | EVQLVQSGAEVKKPGESLKISCKGSGYSF |
| 2368 | | | | | SARS-CoV2 | RBD | Human Patient | | TTYWIGWVRQMPGKGLEWMGIIYPGD |
| | | | | | | | | | SDTRYSPSFQGQVTISADKSISTAYLQWS |
| | | | | | | | | | SLKASDTAMYYCARRRGGIGIEYGMDV |
| | | | | | | | | | WGQGTTVTVSS |
| COV2- | Ab | SARS-CoV2 | SARS-CoV1 | | SARS-CoV1, | S; non- | B-cells; SARS-CoV2 | 400 | QVQLVESGGGVVQPGRSLRLSCAASGF |
| 2369 | | | | | SARS-CoV2 | RBD | Human Patient | | TFSSYAMHWVRQAPGKGLEWVADISY |
| | | | | | | | | | DGSEKYYADSVKGRFTIYRDNSKNTLYLQ |
| | | | | | | | | | MNSLRAEDTAVYYCAKDFGGDNTAMV |
| | | | | | | | | | EYFFDFWGQGTLVTVSS |
| COV2- | Ab | SARS-CoV2 | SARS-CoV1 | | SARS-CoV1, | S; RBD | B-cells; SARS-CoV2 | 401 | QVQLQESGPGLVKPSETLSLTCTVSGGS |
| 2370 | | | | | SARS-CoV2 | | Human Patient | | VSSGSYYWSWIRQPPGKGLECIGYIYYSG |
| | | | | | | | | | SSNYNPSLKSRVTISVDTSKNQFSLKMSS |
| | | | | | | | | | VTAADTAVYYCAGSPVPPTIVGASYWG |
| | | | | | | | | | QGTLVTDSS |
| COV2- | Ab | SARS-CoV1, | | | SARS-CoV2 | S; non- | B-cells; SARS-CoV2 | 402 | QVQLVESGGGVVQPGRSLRLSCAASGF |
| 2371 | | SARS-CoV2 | | | | RBD | Human Patient | | TFSNYGMHWVRQAPGKGLEWVAVISY |
| | | | | | | | | | DGTNKYYADSVKGRFTISRDNSKNTLYL |
| | | | | | | | | | QMNSLRADDTAVYYCAKGRGNYLTFFD |
| | | | | | | | | | SWGQGTLVTVSS |
| COV2- | Ab | SARS-CoV2 | SARS-CoV1 | | SARS-CoV1, | S; non- | B-cells; SARS-CoV2 | 403 | EVQLVESGGGLVQPGGSLRLSCAASGLT |
| 2373 | | | | | SARS-CoV2 | RBD | Human Patient | | VSSNYMSWVRQAPGKGLEWVSVIYSG |
| | | | | | | | | | GSTYYADSVKGRFTISGDNSKNTLYLQM |
| | | | | | | | | | NSLRVDDTAVYYCARDPGSRYSGGWYD |
| | | | | | | | | | YYYAMDVWGQGTTVTVSS |
| COV2- | Ab | SARS-CoV2 | SARS-CoV1 | | SARS-CoV1, | S; non- | B-cells; SARS-CoV2 | 404 | EVQLVQSGAEVKKPGESLKISCKGSGYSF |
| 2378 | | | | | SARS-CoV2 | RBD | Human Patient | | PTYWIGWVRQMPGKGLEWMGIIYPGD |
| | | | | | | | | | SDTRYGPSFQGQVTISADKSISTAYLQWS |
| | | | | | | | | | SLKASDTAMYYCARRDTDFDYWGQGTL |
| | | | | | | | | | VTVSS |
| COV2- | Ab | SARS-CoV2 | SARS-CoV1 | SARS-CoV2 | | S; RBD | B-cells; SARS-CoV2 | 405 | QMQLVQSGPEVKKPGTSVKVSCKASGF |
| 2381 | | | | | | | Human Patient | | TFTSSAVQWVRQARGQRLEWIGWIAV |
| | | | | | | | | | GSGNTNYAQKFQERVSITRDMSTSTAY |
| | | | | | | | | | MELSSLRSEDTAVYYCAAPYCSRTSCHD |
| | | | | | | | | | AFDIWGQGTKVTVSS |
| COV2- | Ab | SARS-CoV1, | | | SARS-CoV2 | S; RBD | B-cells; SARS-CoV2 | 406 | QVQLVESGGGVVQPGRSLRLSCVASGF |
| 2382 | | SARS-CoV2 | | | | | Human Patient | | TFSSYGMHWVRQAPGKGLEWVAVISF |
| | | | | | | | | | DGSNKYYADSVKGRFTISRDNSKNTLYL |
| | | | | | | | | | QMNSLRAEDTAVFYCAKDLPPYASGWY |
| | | | | | | | | | EGGFDYWGRGTQVTVSS |
| COV2- | Ab | SARS-CoV2 | SARS-CoV1 | | SARS-CoV1, | S; non- | B-cells; SARS-CoV2 | 407 | QVQLVESGGGVVQPGRSLRLSCAASGF |
| 2383 | | | | | SARS-CoV2 | RBD | Human Patient | | TFSSYGMHWVRQAPGKGLEWVAVVSY |
| | | | | | | | | | DGSNKYYTDSVKGRFTISRDNSKNTLFLQ |
| | | | | | | | | | MIGLREEDTAVYYCAQGRGGYYSPFDD |
| | | | | | | | | | WGQGTLVTVSS |
| COV2- | Ab | SARS-CoV2 | SARS-CoV1 | | SARS-CoV1, | S; NTD | B-cells; SARS-CoV2 | 408 | QVTLKESGPVLVKPTETLTLTCTVSGFSL |
| 2384 | | | | | SARS-CoV2 | | Human Patient | | SNARMGVSWIRQPPGKALEWLAHIFSG |
| | | | | | | | | | DEKSYSTSLKSRLTISKDTSKSQVVLTMT |
| | | | | | | | | | NMDPLDTATYYCARTTWGTWIQAWYF |
| | | | | | | | | | DIWGRGTLVTVSS |
| COV2- | Ab | SARS-CoV2 | SARS-CoV1 | | SARS-CoV1, | S; non- | B-cells; SARS-CoV2 | 409 | QVQLVESGGGVVQPGRSLRLSCAASGF |
| 2386 | | | | | SARS-CoV2 | RBD | Human Patient | | TFSSYGMHWVRQAPGKGLEWVAVIWF |
| | | | | | | | | | DGSNKHYADSVKGRFTISRDNSKNTLYL |
| | | | | | | | | | QMNSLRAEDTAVYYCAREGDFWSGYYT |
| | | | | | | | | | GWFDPWGQGTLVTVSS |
| COV2- | Ab | SARS-CoV1, | | | SARS-CoV2 | S; non- | B-cells; SARS-CoV2 | 410 | QVQLVESGGGVVQPGRSLRLSCAASGF |
| 2387 | | SARS-CoV2 | | | | RBD | Human Patient | | TFSSYGMHWVRQAPGKGLEWVAVISY |
| | | | | | | | | | DGSNKYYADSVKGRFTISRDNSKNTLYL |
| | | | | | | | | | QMNSLRAEDTAVYYCAKDLTIVVIPAAP |
| | | | | | | | | | NFDYWGQGTLVTVSS |
| COV2- | Ab | SARS-CoV1, | | | SARS-CoV2 | S; non- | B-cells; SARS-CoV2 | 411 | EVQLLESGGGLVQPGGSLRLSCAASGFT |
| 2388 | | SARS-CoV2 | | | | RBD | Human Patient | | FSSYAMSWVRQAPGKGLEWVSFISGTG |
| | | | | | | | | | DSTYYADSVKGRFTISRDNSKNTLYLQM |
| | | | | | | | | | NSLRAEDTAVYYCAKDQARVQDYIWGS |
| | | | | | | | | | YRSYGMDVWGLGTTVTVSS |
| COV2- | Ab | SARS-CoV2 | SARS-CoV1 | SARS-CoV2 | | S; RBD | B-cells; SARS-CoV2 | 412 | EVQLVESGGGLVQPGRSLRLSCAASGFT |
| 2389 | | | | | | | Human Patient | | FDDYAMHWVRQAPGKGLEWVSGISW |
| | | | | | | | | | NSGTIGYADSVKGRFIISRDNAKNSLYLQ |
| | | | | | | | | | MNSLRPEDTALYYCAKDIIRQGEDGMD |
| | | | | | | | | | VWGQGTTVTVSS |
| COV2- | Ab | SARS-CoV2 | SARS-CoV1 | SARS-CoV2 | | S; RBD | B-cells; SARS-CoV2 | 413 | QVQLVQSGAEVKKPGASVKVSCKASGY |
| 2391 | | | | | | | Human Patient | | TFGSFDINWVRQATGQGLEWMGRMN |
| | | | | | | | | | SNSGNTAYAQKFQGRVTMTRDTSTNTA |
| | | | | | | | | | YMELSSLRSEDTAMYYCARMRSGWPTH |
| | | | | | | | | | GRPDDFWGRGTLVTVSS |
| COV2- | Ab | SARS-CoV2 | SARS-CoV1 | | SARS-CoV1, | S; NTD | B-cells; SARS-CoV2 | 414 | EVQLVESGGGLVKPGGSLRLSCAASGFT |
| 2394 | | | | | SARS-CoV2 | | Human Patient | | FSGYSMNWVRQAPGKGLEWVSSISSSS |
| | | | | | | | | | SYIYYADSVKGRFTISRDNAKNSLYLQMN |
| | | | | | | | | | SLRAEDTAVYYCARWLQLRSDYYYFGM |
| | | | | | | | | | DVWGQGTTVTVSS |
| COV2- | Ab | SARS-CoV1, | | | SARS-CoV2 | S; non- | B-cells; SARS-CoV2 | 415 | EVQLVESGGGLVQPGGSLRLSCVASGFT |
| 2397 | | SARS-CoV2 | | | | RBD | Human Patient | | FSFYWMSWVRQAPGKGLEWVANIKQ |
| | | | | | | | | | DGGEKYYVDSVKGRFTISRDNAKNSLYL |
| | | | | | | | | | QMNSLRAEDTAVYYCARLSGSSWDFDY |
| | | | | | | | | | WGQGTLVTVSS |
| COV2- | Ab | SARS-CoV2 | SARS-CoV1 | | SARS-CoV1, | S; RBD | B-cells; SARS-CoV2 | 416 | EVQLVESGGGLVQPGGSLRLSCAASGFS |
| 2399 | | | | | SARS-CoV2 | | Human Patient | | VSTNYMSWVRQAPGKGLEWVSVIYSG |
| | | | | | | | | | GSTFYADSVKGRFTISRDNSKNTLYLQM |
| | | | | | | | | | NSLRAEDTAVYYCARDYRDWIWGQGTL |
| | | | | | | | | | VTVSS |
| COV2- | Ab | SARS-CoV2 | SARS-CoV1 | | SARS-CoV1, | S; non- | B-cells; SARS-CoV2 | 417 | QITLKESGPTLVKPTQTLTLTCTFSGFSL |
| 2400 | | | | | SARS-CoV2 | RBD | Human Patient | | STSGVGVGWIRQPPGKALEWLALIYWD |
| | | | | | | | | | DDKRYSPSLKSRLTITKDTSKNQVVLTMT |
| | | | | | | | | | NMDPVDTATYYCAHNRFQYCSSTTCYTL |
| | | | | | | | | | LPFDYWGQGTLVTVSS |
| COV2- | Ab | SARS-CoV1, | | | SARS-CoV2 | S; non- | B-cells; SARS-CoV2 | 418 | QVQLVESGGGVVQPGRSLRLSCAASGF |
| 2401 | | SARS-CoV2 | | | | RBD | Human Patient | | TFSSYALFWVRQAPGKGLEWVAVISYD |
| | | | | | | | | | GNNKYYADSVRGRFTISRDNSKNTLYLQ |
| | | | | | | | | | MNSLRPEDTAVYYCARPYTGSYKSYMD |
| | | | | | | | | | VWGKGTTVTVSS |
| COV2- | Ab | SARS-CoV1, | | | SARS-CoV2 | S; non- | B-cells; SARS-CoV2 | 419 | QVQLVESGGGVVQPGRSLRLSCAASGF |
| 2403 | | SARS-CoV2 | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMSSLRAEDTAVFYCARGDGDVYNFLL |
| | | | | | | | | | VRNWFDPWGQGTLVTVSS |
| COV2- | Ab | SARS-CoV2 | SARS-CoV1 | | SARS-CoV1, | S; NTD | B-cells; SARS-CoV2 | 420 | QLQLQESGPGLVKPSETLSLTCTVSGGSI |
| 2405 | | | | | SARS-CoV2 | | Human Patient | | SSSSYYWGWIRQPPGKGLEWIGSFYYSG |
| | | | | | | | | | STYYNPSLKSRVTISVDTSKNQFSLNLSS |
| | | | | | | | | | VTAADTAVYSCASLWFGDLYSFDYWGQG |
| | | | | | | | | | TLVTVSS |
| COV2- | Ab | SARS-CoV1, | | | SARS-CoV2 | S; non- | B-cells; SARS-CoV2 | 421 | EVQLVESGGGLVQPGGSLRLSCADSAFT |
| 2406 | | SARS-CoV2 | | | | RBD | Human Patient | | FSSFWMSWVRQAPGKGLEWVANIKQ |
| | | | | | | | | | DGSEKFYLDSVKGRFTISRDNAKNSLYLQ |
| | | | | | | | | | MNSLRAEDTAVYYCARLGRSSWNFDY |
| | | | | | | | | | WGQGTLVTVSS |
| COV2- | Ab | SARS-CoV1, | | | SARS-CoV2 | S; non- | B-cells; SARS-CoV2 | 422 | QVQLQESGPGLVKPSQTLSLTCTVSGDSI |
| 2408 | | SARS-CoV2 | | | | RBD | Human Patient | | SSGSYYWSWIRQPAGKGLEWIGRIYTSG |
| | | | | | | | | | STTYNPSLKSRVTISVNTSKNQFSLNLSS |
| | | | | | | | | | VTAADTAVYYCARVGGISPYYYYYYMDV |
| | | | | | | | | | WGKGTTVTVSS |
| COV2- | Ab | SARS-CoV2 | SARS-CoV1 | SARS-CoV2 | | S; RBD | B-cells; SARS-CoV2 | 423 | QVQLVQSGAEVKKPGASVKVSCKASGY |
| 2413 | | | | (weak) | | | Human Patient | | TFTSYDINWVRQATGQGLEWMGWMN |
| | | | | | | | | | PNSGNAGYGQKFQGRVTMTRNTSISTA |
| | | | | | | | | | YMELSSLRSEDTAVYYCARMRSGWPTH |
| | | | | | | | | | GRPDDYWGRGTLVTVSS |
| COV2- | Ab | SARS-CoV2 | SARS-CoV1 | | SARS-CoV1, | S; RBD | B-cells; SARS-CoV2 | 424 | QVQLQESGPGLVKPSQTLSLTCTVSGGSI |
| 2416 | | | | | SARS-CoV2 | | Human Patient | | SSGGYYWSWIRQHPGKGLEWIGYIYYS |
| | | | | | | | | | GSTYYNPSLKSRVTISVDTSKNQFSLKLS |
| | | | | | | | | | SVTAADTAVYYCASAKLVATISYFDYWGQ |
| | | | | | | | | | GTLVTVSS |
| COV2- | Ab | SARS-CoV1, | | | SARS-CoV2 | S; RBD | B-cells; SARS-CoV2 | 425 | QVQLQQWGAGLLKPSETLSLTCAVYGG |
| 2417 | | SARS-CoV2 | | | | | Human Patient | | SFSGYYWNWLRQPPGKGLEWIGEINHS |
| | | | | | | | | | GSTNYNPSLKSRVTISVDTSKNQFSLKLS |
| | | | | | | | | | SVTAADTAVYYCARVGGYYYYYMDVWG |
| | | | | | | | | | KGTTVTVSS |
| COV2- | Ab | SARS-CoV2 | SARS-CoV1 | | SARS-CoV1, | S; non- | B-cells; SARS-CoV2 | 426 | EVRLLESGGGLVQPGGSLRLSCAASGFT |
| 2418 | | | | | SARS-CoV2 | RBD | Human Patient | | FSDYAMNWVRQAPGKGLEWVSAISAT |
| | | | | | | | | | GGSTFYADSVKGRFSISRDNSKNSLVLQ |
| | | | | | | | | | MNSLRAEDTAVYYCAKPYGMDVWGQ |
| | | | | | | | | | GTTVTVSS |
| COV2- | Ab | SARS-CoV2 | SARS-CoV1 | | SARS-CoV1, | S; non- | B-cells; SARS-CoV2 | 427 | QGQLVQSGGDVVQPGKSLRLSCAASGF |
| 2420 | | | | | SARS-CoV2 | RBD | Human Patient | | TFTNYAMHWVRQAPGKGLEWVAVISN |
| | | | | | | | | | DGSNEKYVDSVKGRFSLSRDNSKNTVYL |
| | | | | | | | | | DMHSLRPEDTAIYYCARDRSNLERLVMT |
| | | | | | | | | | FGGIIAGAFDIWGQGARVTVSS |
| COV2- | Ab | SARS-CoV1, | | | SARS-CoV2 | S; non- | B-cells; SARS-CoV2 | 428 | QVQLVESGGGVVQPGRSLRLSCAASGF |
| 2422 | | SARS-CoV2 | | | | RBD | Human Patient | | TFSSYAMYWVRQAPGKGLEWVAVISYD |
| | | | | | | | | | GINKYYADSVKGRFTISRDNSKNTLYLQ |
| | | | | | | | | | MNSLRAEDTAVYYCARVNSGSYYSYFDY |
| | | | | | | | | | WGQGTLVTVSS |
| COV2- | Ab | SARS-CoV2 | SARS-CoV1 | | SARS-CoV1, | S; non- | B-cells; SARS-CoV2 | 429 | QVQLQESGPGLVKPSGTLSLTCAVSGGS |
| 2427 | | | | | SARS-CoV2 | RBD | Human Patient | | ISSSNWWSWVRQPPGKGLEWIGEIYHS |
| | | | | | | | | | GSTNYNPSLKSRVTISVDKSKNQFSLKLN |
| | | | | | | | | | SVTAADTAVYYCASRWGDYFDSSGAYD |
| | | | | | | | | | SWGQGTLLTVSS |
| COV2- | Ab | SARS-CoV2 | SARS-CoV1 | | SARS-CoV1, | S; non- | B-cells; SARS-CoV2 | 430 | QVQLVESGGGVVQPGKSLRLSCAASGF |
| 2428 | | | | | SARS-CoV2 | RBD | Human Patient | | TFSSYGMHWVRQAPGKGLEWVAVIWY |
| | | | | | | | | | DGNNKFYVDSVKGRFTISRDNSKNTLY |
| | | | | | | | | | MEMNSLRAEDTAVYYCARKGPLWRFD |
| | | | | | | | | | YWGQGTLVTVSS |
| COV2- | Ab | SARS-CoV1, | | | SARS-CoV2 | S; non- | B-cells; SARS-CoV2 | 431 | EVQLVESGGGLIQPGGSLRLSCAASGFIV |
| 2429 | | SARS-CoV2 | | | | RBD | Human Patient | | SSNYMSWVRQAPGKGLEWVSVIYSGG |
| | | | | | | | | | STYYADSVKGRFTISRDNSKNTLYLQMN |
| | | | | | | | | | SLRAEDTAVYYCARESTQWGQGTLVTV |
| | | | | | | | | | SS |
| COV2- | Ab | SARS-CoV1, | | | SARS-CoV2 | S; non- | B-cells; SARS-CoV2 | 432 | QLQLQESGPGLVKPSETLSLTCTVSGGSI |
| 2430 | | SARS-CoV2 | | | | RBD | Human Patient | | SSSSYYWGWIRQPPGKGLEWIGSVYYIG |
| | | | | | | | | | STYYNPSLKSRVTMSVDTSKNQFSLKLSS |
| | | | | | | | | | VTAADTAVYYCARAPFQLLDKYYFFYYM |
| | | | | | | | | | DVWGKGTTVTVSS |
| COV2- | Ab | SARS-CoV2 | SARS-CoV1 | | SARS-CoV1, | S; RBD | B-cells; SARS-CoV2 | 433 | EVQLVESGGDLIQPGGSLRLSCAASGLT |
| 2434 | | | | | SARS-CoV2 | | Human Patient | | VSSNYMSWVRQAPGKGLEWVSIIYSGG |
| | | | | | | | | | STYYADSVKGRFSISRDNSNNTLYLQMN |
| | | | | | | | | | SLRAEDTAVYFCARHIPAWGYKWGQGT |
| | | | | | | | | | LVTVSS |
| COV2- | Ab | SARS-CoV2 | SARS-CoV1 | | SARS-CoV1, | S; NTD | B-cells; SARS-CoV2 | 434 | EVQLLESGGGLVQPGGSLRLSCAASGFT |
| 2438 | | | | | SARS-CoV2 | | Human Patient | | FTSYGMSWVRQAPGKGLEWVSAISISG |
| | | | | | | | | | GSTYYADSVKGRFTISRDNSKNTLYLQM |
| | | | | | | | | | NSLRAEDTAVYYCAKLLGSGITLDNDAF |
| | | | | | | | | | DIWGQGTMVTVSS |
| COV2- | Ab | SARS-CoV1, | | | SARS-CoV2 | S; non- | B-cells; SARS-CoV2 | 435 | QVQLVQSGAEVKKPGSSVKVSCKASGG |
| 2441 | | SARS-CoV2 | | | | RBD | Human Patient | | TFSSYAIIWVRQAPGQGLEWMGGIIPIF |
| | | | | | | | | | GTTNYAQKFQGRVTITADESTSTAYVELS |
| | | | | | | | | | SLRSEDTAVYYCARIGHFDSSGYYLDYW |
| | | | | | | | | | GQGTLVTVSS |
| COV2- | Ab | SARS-CoV2 | SARS-CoV1 | | SARS-CoV1, | S; NTD | B-cells; SARS-CoV2 | 436 | EVQLLESGGGLVQPGGSLRLSCAASGFT |
| 2444 | | | | | SARS-CoV2 | | Human Patient | | FTSYAMNWVRQAPGKGLEWVSAISVS |
| | | | | | | | | | GGSTYYADSVKGRFTISRDNSKNTLYLQ |
| | | | | | | | | | MNSLRAEDTAVYYCAKDFGSGIVGATG |
| | | | | | | | | | FDFWGQGTLVTVSS |
| COV2- | Ab | SARS-CoV2 | SARS-CoV1 | | SARS-CoV1, | S; non- | B-cells; SARS-CoV2 | 437 | EVQLVESGGGVVRPGGSLRLSCAASGFT |
| 2445 | | | | | SARS-CoV2 | RBD | Human Patient | | FDDYGMSWVRQAPGKGLEWVSAINW |
| | | | | | | | | | NGGSTGYADSVKGRFTISRDNAKNSLYL |
| | | | | | | | | | QMNSLRAEDTALYHCARRRSSSRYSSG |
| | | | | | | | | | WYMYYYYMDVWGKGTTVTVSS |
| COV2- | Ab | SARS-CoV2 | SARS-CoV1 | | SARS-CoV1, | S; non- | B-cells; SARS-CoV2 | 438 | EVQLVQSGAEVKKPGESLKISCKGSGYSF |
| 2446 | | | | | SARS-CoV2 | RBD | Human Patient | | TNSWIGWVRQMPGKGLEWMGIIYPG |
| | | | | | | | | | DSDTRYSPSFQGQVTISADKSISTAYLQW |
| | | | | | | | | | SSLKASDTAIYYCATHRCSGGFCYLAYWG |
| | | | | | | | | | QGTLVTVSS |
| COV2- | Ab | SARS-CoV1, | | | SARS-CoV2 | S; non- | B-cells; SARS-CoV2 | 439 | QVQLVESGGGVVQPGRSLRLSCATSGFT |
| 2449 | | SARS-CoV2 | | | | RBD | Human Patient | | FSSFALHWVRQAPGKGLEWVTVISDDG |
| | | | | | | | | | NNKYYVDSVKGRFTISRDNSKNTLFLQM |
| | | | | | | | | | NSLRVEDTAIYYCARASYNSNWSIGEYFR |
| | | | | | | | | | DWGQGTLVTVSS |
| COV2- | Ab | SARS-CoV1, | | | SARS-CoV2 | S; RBD | B-cells; SARS-CoV2 | 440 | QVQLVESGGGVVQPGRSLRLSCAASGF |
| 2450 | | SARS-CoV2 | | | | | Human Patient | | TFSTYGMHWVRQAPGKGLEWVAVILY |
| | | | | | | | | | DGSNRYYADSVKGRFTISRDNSKNTLYL |
| | | | | | | | | | QMNSLRAEDTAVYYCAKQGGLYCSGTN |
| | | | | | | | | | CWGGYLDYWGQGTLVTVSS |
| COV2- | Ab | SARS-CoV2 | SARS-CoV1 | | SARS-CoV1, | S; non- | B-cells; SARS-CoV2 | 441 | QITLKESGPTLVKPTQTLTLTCTVSGFSL |
| 2451 | | | | | SARS-CoV2 | RBD | Human Patient | | STSGVGVGCIRQPPGKALEWLALIYWDD |
| | | | | | | | | | DKRYSPSLKSRLTITRDTSKNQVVLTMTN |
| | | | | | | | | | MDPVDTGTYFCVHRHVSGAFDYWGQG |
| | | | | | | | | | TLVTVSS |
| COV2- | Ab | SARS-CoV1, | | | SARS-CoV2 | S; non- | B-cells; SARS-CoV2 | 442 | QVQLVQSGSELKKPGASVKVSCKASGYT |
| 2453 | | SARS-CoV2 | | | | RBD | Human Patient | | FTSYAMNWVRQAPGQGLEWMGWINT |
| | | | | | | | | | NTGNPTYAQGFTGRFVFSLDTSVNTAYL |
| | | | | | | | | | QISSLKAEDTAVYYCARARLLGYCSSTSC |
| | | | | | | | | | YTIGWGAFDIWGQGTMVTVSS |
| COV2- | Ab | SARS-CoV2 | SARS-CoV1 | | SARS-CoV1, | S; non- | B-cells; SARS-CoV2 | 443 | QVQLVESGGGVVQPGRSLRLSCAASGF |
| 2454 | | | | | SARS-CoV2 | RBD | Human Patient | | TFSSYGMHWVRQAPGKGLEWVAVIWY |
| | | | | | | | | | DGSNKYYADSVKGRFTISRDNSKNTLYL |
| | | | | | | | | | QMNSLRAEDTAVYYCAREGQGTYLDY |
| | | | | | | | | | WGQGTLVTVSS |
| COV2- | Ab | SARS-CoV2 | SARS-CoV1 | | SARS-CoV1, | S; NTD | B-cells; SARS-CoV2 | 444 | EVQLVESGGGLVQPGGSLRLSCAASGFT |
| 2455 | | | | | SARS-CoV2 | | Human Patient | | FSSYEMNWVRQAPGKGLEWVSYISSSG |
| | | | | | | | | | SAIYYADSVKGRFTISRDNAKNSLYLQM |
| | | | | | | | | | NSLRVEDTAVYYCAREARSRYFDWLPSY |
| | | | | | | | | | YFDYWGQGTLVTVSS |
| COV2- | Ab | SARS-CoV1, | | | SARS-CoV2 | S; non- | B-cells; SARS-CoV2 | 445 | QVQLVESGGGVVQPGRSLRLSCAASGF |
| 2458 | | SARS-CoV2 | | | | RBD | Human Patient | | TFSRHAMHWVRQAPGKGLEWVAVISY |
| | | | | | | | | | DGSNKYYADSVKGRFAISRDNSKNTLYL |
| | | | | | | | | | QMNSLRPEDTAVYYCARDPSPLVLITSID |
| | | | | | | | | | YWGQGTLVTVSS |
| COV2- | Ab | SARS-CoV2 | SARS-CoV1 | | SARS-CoV1, | S; NTD | B-cells; SARS-CoV2 | 446 | QLQLQESGPGLVKPSETLSLTCTVSGGSI |
| 2459 | | | | | SARS-CoV2 | | Human Patient | | SSGTYYCGWIRQPPGKGLEWIGSTYYG |
| | | | | | | | | | GSTLYNPSLRGRVTISVDTSKNQFSLKLS |
| | | | | | | | | | SVTAADTAVYYCARRGNYYDSKNWFDP |
| | | | | | | | | | WGQGTLVTVSS |
| COV2- | Ab | SARS-CoV1, | | | SARS-CoV2 | S; RBD | B-cells; SARS-CoV2 | 447 | QVQLQESGPGLVKPSQTLSLTCTVSGGSI |
| 2461 | | SARS-CoV2 | | | | | Human Patient | | SSGGYFWSWIRQHPGKGLEWIGSIYYS |
| | | | | | | | | | GSTYYNPSLRSRITISVDTSKNQFSLKLS |
| | | | | | | | | | SVTAADTAVYYCARGGSGSYSLFDYWGQ |
| | | | | | | | | | GTLVTVSS |
| COV2- | Ab | SARS-CoV1, | | | SARS-CoV2 | S; RBD | B-cells; SARS-CoV2 | 448 | EVQLVESGGGLVQPGGSLRLSCAASGFT |
| 2462 | | SARS-CoV2 | | | | | Human Patient | | FSSSDLHWVRQATGKGLEWVSAIGTAG |
| | | | | | | | | | DTYYLGSVKGRFTISRENGKNSLYLQMN |
| | | | | | | | | | SLRAGDTAVYYCARVLYDSSGFYNWFDP |
| | | | | | | | | | WGQGTLVTVSS |
| COV2- | Ab | SARS-CoV1, | | | SARS-CoV2 | S; RBD | B-cells; SARS-CoV2 | 449 | QVQLQESGPGLVKPSQTLSLTCTVSGGSI |
| 2464 | | SARS-CoV2 | | | | | Human Patient | | SSGGYYWSWIRQHPGKGLEWIGYISYS |
| | | | | | | | | | GSTYYNPSLKSRLTISVDTSKNQFSLKLS |
| | | | | | | | | | SVTAADTAVYYCARDLGDGYNLRVPAYF |
| | | | | | | | | | DLWGRGTLVTVSS |
| COV2- | Ab | SARS-CoV1, | | | SARS-CoV2 | S; RBD | B-cells; SARS-CoV2 | 450 | QVQLQESGPGLVKPSGTLSLTCAVSGGS |
| 2465 | | SARS-CoV2 | | | | | Human Patient | | ISSSNWWSWVRQPPGKGLEWIGEIYHG |
| | | | | | | | | | GSTDYNPSLKSRVTISVDKSKNQFSLKLT |
| | | | | | | | | | SVTAADTAVYYCARVDHVNVRDYWGPG |
| | | | | | | | | | TLVTVSS |
| COV2- | Ab | SARS-CoV2 | SARS-CoV1 | | SARS-CoV1, | S; NTD | B-cells; SARS-CoV2 | 451 | EVQLVESGGGLVKPGGSLRLSCAASGFT |
| 2466 | | | | | SARS-CoV2 | | Human Patient | | FSSYSMNWVRQAPGKGLEWVSSISNSN |
| | | | | | | | | | SFIYYADSMKGRFTISRDNAKNSLYLQM |
| | | | | | | | | | NSLRAEDTAVYYCARVNGNSNWNFGSY |
| | | | | | | | | | YYYYMDVWGKGTTVTVSS |
| COV2- | Ab | SARS-CoV2 | SARS-CoV1 | | SARS-CoV1, | S; non- | B-cells; SARS-CoV2 | 452 | QVQLVESGGGVVQPGRSLRLSCAASGF |
| 2473 | | | | | SARS-CoV2 | RBD | Human Patient | | TFSNYGMHWVRQAPGKGLEWVAVIYY |
| | | | | | | | | | DGSNKYYADSVKGRFTISRDNSKNTLYL |
| | | | | | | | | | QIHSLRAEDTAVYYCAREGQMAATTGID |
| | | | | | | | | | YWGQGTLVTVSS |
| COV2- | Ab | SARS-CoV2 | SARS-CoV1 | | SARS-CoV1, | S; non- | B-cells; SARS-CoV2 | 453 | EVQLVESGGGLVKPGGSLRLSCAASGFT |
| 2474 | | | | | SARS-CoV2 | RBD | Human Patient | | FSNAWMSWVRQAPGKGLEWVGRIKG |
| | | | | | | | | | KTDGGTTDYAAPVKGRFTISKDDSKNTL |
| | | | | | | | | | YLQMSSLNTEDTAVYWCTTLTYYYDSSA |
| | | | | | | | | | YLNDAFDIWGQGTMVTVSS |
| COV2- | Ab | SARS-CoV1, | | | SARS-CoV2 | S; non- | B-cells; SARS-CoV2 | 454 | EVQLVESGGGLVQPGRSLRLSCAASGFT |
| 2478 | | SARS-CoV2 | | | | RBD | Human Patient | | FDDYAMHWVRQAPGKGLEWVSGISW |
| | | | | | | | | | NSDNIGYADSVKGRFTISRDIAKNSLYLQ |
| | | | | | | | | | MNSLRAEDTALYYCAKGIYYDIFMPLLD |
| | | | | | | | | | WGRGTLVTVSS |
| COV2- | Ab | SARS-CoV2 | SARS-CoV1 | SARS-CoV2 | | S; RBD | B-cells; SARS-CoV2 | 455 | QVQLVQSGAEVKKPGSSVKVSCKTSGD |
| 2479 | | | | | | | Human Patient | | TSSSYTVGWVRQAPGQGLEWMGRIIPI |
| | | | | | | | | | LGIAYSAQKFQGRLTITADKSTSTSYMEL |
| | | | | | | | | | SSLRSEDTAVYYCARGVVAATPGWFDP |
| | | | | | | | | | WGQGTLVTVSS |
| COV2- | Ab | SARS-CoV1, | | | SARS-CoV2 | S; non- | B-cells; SARS-CoV2 | 456 | QVQLVQSGAEVKKPGSSVKVSCKASGG |
| 2481 | | SARS-CoV2 | | | | RBD | Human Patient | | TFGSYVISWVRQAPGQGLQWMGGIIPI |
| | | | | | | | | | FGKPNYAQKFQGRVTITADESTSTAYME |
| | | | | | | | | | LSSLRSEDTAVYYCARGWFGELLKGTYW |
| | | | | | | | | | FDPWGQGTLVTVSS |
| COV2- | Ab | SARS-CoV2 | SARS-CoV1 | SARS-CoV2 | | S; RBD | B-cells; SARS-CoV2 | 457 | QVQLVQSGAEVKKPGSSVKVSCKASGG |
| 2485 | | | | (weak) | | | Human Patient | | TFSSYSITWVRQAPGQGLEWMGRIIPVL |
| | | | | | | | | | GIANYAQKFQDRVTITADKSTSTAYMEL |
| | | | | | | | | | SSLRSEDTAVYYCARVGVSGFKSGSNWY |
| | | | | | | | | | FDLWGRGTLVTVSS |
| COV2- | Ab | SARS-CoV2 | SARS-CoV1 | | SARS-CoV1, | S; non- | B-cells; SARS-CoV2 | 458 | EVQLVESGGGLVQPGGSLRLSCAVSGFT |
| 2488 | | | | | SARS-CoV2 | RBD | Human Patient | | FSSYWMHWVRQAPGKGLVWVSRINSD |
| | | | | | | | | | GSSTSYADSVKGRFTISRDNAKNTLYLQ |
| | | | | | | | | | MNSLRAEDTAVYYCAREVEQLAHMVDY |
| | | | | | | | | | WGQGTLVTVSS |
| COV2- | Ab | SARS-CoV2 | SARS-CoV1 | SARS-CoV2 | | S; NTD | B-cells; SARS-CoV2 | 459 | QLQLQESGPGLVKPSETLSLTCTVSGGSI |
| 2489 | | | | (weak) | | | Human Patient | | SSTTYYWGWIRQPPGKGLEWIASIYYSG |
| | | | | | | | | | STYYNPSLKSRLTVSVDTSKNQFSLKLSS |
| | | | | | | | | | VTAADTAVYYCARQWKWFGEAWYFDL |
| | | | | | | | | | WGRGTLVTVSS |
| COV2- | Ab | SARS-CoV2 | SARS-CoV1 | | SARS-CoV1, | S; NTD | B-cells; SARS-CoV2 | 460 | EVQLVESGGGLVQPGGSLRLSCAASGFT |
| 2490 | | | | | SARS-CoV2 | | Human Patient | | FSSYWMNWVRQAPGKGLEWVANINQ |
| | | | | | | | | | DGGEKYYVDSVRGRFTISRDNAKNSLYL |
| | | | | | | | | | QMNSLRAEDTAVYYCARDPYDLYGDYG |
| | | | | | | | | | GTFDYWGQGTLVTVSS |
| COV2- | Ab | SARS-CoV2 | SARS-CoV1 | | SARS-CoV1, | S; NTD | B-cells; SARS-CoV2 | 461 | QVQLVQSGAAVKKPGSSVKVSCKASGG |
| 2495 | | | | | SARS-CoV2 | | Human Patient | | TFSSYAISWVRQAPGQGLEWMGGIVPI |
| | | | | | | | | | FGTANYAQKFQGRVTITADESTTTAYME |
| | | | | | | | | | LNSLRSEDTAVYYCAREDYYGSGSLVDPY |
| | | | | | | | | | YYYRMDVWGQGTTVTVSS |
| COV2- | Ab | SARS-CoV2 | SARS-CoV1 | | SARS-CoV1, | S; non- | B-cells; SARS-CoV2 | 462 | QVQLVESGGGVVQPGRSLRLSCAASGF |
| 2496 | | | | | SARS-CoV2 | RBD | Human Patient | | TFSNYGMHWVRQAPGKGLEWVAVIW |
| | | | | | | | | | YDGSNKYYADSVKGRFTISRDNSKNTLYL |
| | | | | | | | | | QMNSLRAEDTAVYYCVRDLALFEVVIQ |
| | | | | | | | | | QGVWGQGTLVTVSS |
| COV2- | Ab | SARS-CoV2 | SARS-CoV1 | SARS-CoV2 | | S; RBD | B-cells; SARS-CoV2 | 463 | QLQLQESGPGLVKPSETLSLTCTVSGGSV |
| 2499 | | | | | | | Human Patient | | SSRSYYWGWIRQPPGKGLEWIGSIYYSG |
| | | | | | | | | | STYYNPSLKSRVTISVDTSKNQFSLKLSS |
| | | | | | | | | | VTAADTAVYYCARHTVDCGGDCFPNDAF |
| | | | | | | | | | DIWGQGTMVTVSS |
| COV2- | Ab | SARS-CoV2 | SARS-CoV1 | SARS-CoV2 | | S; RBD | B-cells; SARS-CoV2 | 464 | QVQLVQSGAEVKKPGASVKVSCKASGY |
| 2504 | | | | | | | Human Patient | | TFTDYYMHWVRQAPGQGLEWMGWI |
| | | | | | | | | | NPNSRGTNYAQKFQGRVTMTRDTSIST |
| | | | | | | | | | VYMELSRLTSDDTAVYYCARVVVLGYGR |
| | | | | | | | | | PNNYYDGRNVWDYWGQGTLVTVSS |
| COV2- | Ab | SARS-CoV1, | | | SARS-CoV2 | S; non- | B-cells; SARS-CoV2 | 465 | QVQLVESGGGVVQPGRSLRLSCAASGFI |
| 2509 | | SARS-CoV2 | | | | RBD | Human Patient | | FSTYAMHWVRQAPGKGLEWVAVISYD |
| | | | | | | | | | GDNKYYADSVKGRFTISRDNSKNTLYLE |
| | | | | | | | | | MNSLRAEDTAVYYCARPRGGSYQTCFD |
| | | | | | | | | | YWGQGTLVTVSS |
| COV2- | Ab | SARS-CoV2 | SARS-CoV1 | | SARS-CoV1, | S; RBD | B-cells; SARS-CoV2 | 466 | QVQLVQSGSELKKPGASVKVSCKASGYT |
| 2510 | | | | | SARS-CoV2 | | Human Patient | | FTSHTMNWVRQAPGQGLEWMGWIN |
| | | | | | | | | | TNTGNPMYAQGFTGRFVFSLDTSGSTA |
| | | | | | | | | | YLQISSLKAEDTAVYYCARWGPDYGDYA |
| | | | | | | | | | SNDYWGQGTLVTVSS |
| COV2- | Ab | SARS-CoV1, | | SARS-CoV2 | | S; RBD | B-cells; SARS-CoV2 | 467 | EVQLVESGGGVVRPGGSLRLSCAASGFI |
| 2514 | | SARS-CoV2 | | (weak) | | | Human Patient | | FDDYDMTWVRQAPGKGLEWVSGINW |
| | | | | | | | | | NGGSTGYADSVKGRFTISRDNAKNSLYL |
| | | | | | | | | | QMNSLRAEDTALYHCAVIMSPIPRYSGY |
| | | | | | | | | | DWAGDAFDIWGQGTMVTVSS |
| COV2- | Ab | SARS-CoV1, | | | SARS-CoV2 | S; non- | B-cells; SARS-CoV2 | 468 | QVQLVQSGSELKKPGASVKVSCKASGYT |
| 2515 | | SARS-CoV2 | | | | RBD | Human Patient | | FTTYAMNWVRQAPGQGLEWMGWIN |
| | | | | | | | | | TNTGNPTYAQDFTGRFVFSLDTSVSTAYL |
| | | | | | | | | | QISSLKAEDTAVYYCARGLVGRIDPWGQ |
| | | | | | | | | | GTLVTVSS |
| COV2- | Ab | SARS-CoV1, | | | SARS-CoV2 | S; RBD | B-cells; SARS-CoV2 | 469 | QVQLVQSGAEVKKPGASVKVSCKASGY |
| 2516 | | SARS-CoV2 | | | | | Human Patient | | TFTNYYMHWVRQAPGQGLEWMGILN |
| | | | | | | | | | PGAGSTSYAQKFQGRVTMTSDTSTNTV |
| | | | | | | | | | YMQLSSLKSEDTAVYYCARDQQIVPHAD |
| | | | | | | | | | GFDIWGQGTMVTVSS |
| COV2- | Ab | SARS-CoV1, | | | SARS-CoV2 | S; non- | B-cells; SARS-CoV2 | 470 | QVQLVQSGAEVRKPGSSVKVSCKASGG |
| 2517 | | SARS-CoV2 | | | | RBD | Human Patient | | TFSSYAISWVRQAPGQGLEWMGGIIPV |
| | | | | | | | | | FGTANYAQKFQGRVTITADKSTSTAFME |
| | | | | | | | | | LNSLRSEDTAVYYCARIGSYPEYFQHWG |
| | | | | | | | | | QGTLVTVSS |
| COV2- | Ab | SARS-CoV2 | SARS-CoV1 | | SARS-CoV1, | S; RBD | B-cells; SARS-CoV2 | 471 | EVQLVESGGGLVQPGRSLRLSCAASGFT |
| 2518 | | | | | SARS-CoV2 | | Human Patient | | FDDYAMHWVRQAPGKGLEWVSGISW |
| | | | | | | | | | NSGTIGYADSVKGRFIISRDNAKNSLYLQ |
| | | | | | | | | | MNSLRPEDTALYYCAKDIIRQGEDGMD |
| | | | | | | | | | VWGQGTTVTVSS |
| COV2- | Ab | SARS-CoV1, | | | SARS-CoV2 | S; non- | B-cells; SARS-CoV2 | 472 | EVQLVESGGGLVQPGGSLRLSCAASGFT |
| 2520 | | SARS-CoV2 | | | | RBD | Human Patient | | FSSYWMSWVRQAPGKGLEWVANIKED |
| | | | | | | | | | GSEKYYVDSVKGRFTISRDNAKNSLYLQ |
| | | | | | | | | | MNSLRAEDTAVYYCARDVGGYSGYDLG |
| | | | | | | | | | FDYYYYMDVWGKGTTVTVSS |
| COV2- | Ab | SARS-CoV2 | SARS-CoV1 | | SARS-CoV1, | S; non- | B-cells; SARS-CoV2 | 473 | QVQLVESGGGVVQPGRSLRLACAASGF |
| 2521 | | | | | SARS-CoV2 | RBD | Human Patient | | TFSSYGMHWVRQAPGKGLEWVAVISY |
| | | | | | | | | | DGNNKYYADSLKGRFTISRDDSKNTLYL |
| | | | | | | | | | QMNSLRAEDTAVYYCAKDRTAVFLFFGL |
| | | | | | | | | | GDAFDIWGQGTMVTVSS |
| COV2- | Ab | SARS-CoV1, | | | SARS-CoV2 | S; non- | B-cells; SARS-CoV2 | 474 | EVQLVQSGAEVKKPGESLKISCKGSGYSF |
| 2524 | | SARS-CoV2 | | | | RBD | Human Patient | | TSHWIGWVRQMPGKGLEWMGIIYPG |
| | | | | | | | | | DSDTRYSPSFQGQVTISADKSISTAYLQW |
| | | | | | | | | | SSLKASDTAMYYCASALRERGVQLWSV |
| | | | | | | | | | WGQGTLVTVSS |
| COV2- | Ab | SARS-CoV2 | SARS-CoV1 | | SARS-CoV1, | S; NTD | B-cells; SARS-CoV2 | 475 | EVRLLESGGGLVQPGGSLRLSCAASGFT |
| 2525 | | | | | SARS-CoV2 | | Human Patient | | FTSYAMSWVRQAPGKGLQWVSTISVSG |
| | | | | | | | | | GSTYYADSVKGRFTISRDNSKNTLYLQM |
| | | | | | | | | | NSLRAEDTAVYYCAKDPASGIVGPTHFD |
| | | | | | | | | | YWGQGTLVTVSS |
| COV2- | Ab | SARS-CoV2 | SARS-CoV1 | | SARS-CoV1, | S; NTD | B-cells; SARS-CoV2 | 476 | QVQLVQSGAEVKKPGASVKVSCKVSGY |
| 2526 | | | | | SARS-CoV2 | | Human Patient | | TLTELSIHWVRQAPGKGLEWMGGFDP |
| | | | | | | | | | EDAETIYAQQFQGRVTMTEDTSTDTAY |
| | | | | | | | | | MELSSLKSEDTALYYCATGFAVFGRAAV |
| | | | | | | | | | PYWGQGTLVTVSS |
| COV2- | Ab | SARS-CoV2 | SARS-CoV1 | | SARS-CoV1, | S; RBD | B-cells; SARS-CoV2 | 477 | QVQLQESGPGLVKPSQTLSLTCTVSGGSI |
| 2527 | | | | | SARS-CoV2 | | Human Patient | | SSGDYYWSWIRQPPGKGLEWIGYIYYSG |
| | | | | | | | | | STYYNPSLKSRVTISVDTSKNQVSLKLSS |
| | | | | | | | | | VTAADTAVYYCARFRRSYGSGSYYNISFD |
| | | | | | | | | | YWGQGTLVTVSS |
| COV2- | Ab | SARS-CoV2 | SARS-CoV1 | | SARS-CoV1, | S; non- | B-cells; SARS-CoV2 | 478 | QVQLVQSGSELKKPGASVKVSCKASGYT |
| 2529 | | | | | SARS-CoV2 | RBD | Human Patient | | FTRHAMNWVRQAPGQGLEWMGWIN |
| | | | | | | | | | TNTGNPTYAQGFTGRFVFSLDTSVSTAY |
| | | | | | | | | | LQISSLKAEDTAVYYCVREYGSGHPLPIW |
| | | | | | | | | | GQGTLVTVSS |
| COV2- | Ab | SARS-CoV2 | SARS-CoV1 | SARS-CoV2 | | S; RBD | B-cells; SARS-CoV2 | 479 | QVQLQESGPGLVKPSETLSLTCSVSGGSI |
| 2531 | | | | | | | Human Patient | | RSYFWNWVRQPPGKGLEWIGYIYYSGS |
| | | | | | | | | | TNYKPSFKSRVTISLDTSKNQISLKLSSV |
| | | | | | | | | | TAADTAVYYCARATWLRDAFGIWGQGT |
| | | | | | | | | | MVTVSS |
| COV2- | Ab | SARS-CoV2 | SARS-CoV1 | | SARS-CoV1, | S; RBD | B-cells; SARS-CoV2 | 480 | QVQLVQSGAEVKKPGASVRVSCKAPGY |
| 2532 | | | | | SARS-CoV2 | | Human Patient | | TFTTYYIHWVRQAPGQGLEWMGIINPS |
| | | | | | | | | | AGSTTYAQKFQGRVTMTRDTSTSTVYM |
| | | | | | | | | | ELSSLRSEDTAVYYCARGFHVPAALRNW |
| | | | | | | | | | FDPWGQGTLVTVSS |
| COV2- | Ab | SARS-CoV2 | SARS-CoV1 | | SARS-CoV1, | S; NTD | B-cells; SARS-CoV2 | 481 | QVQLQESGPGLVKPSQTLSLTCTVSGGSI |
| 2533 | | | | | SARS-CoV2 | | Human Patient | | SSGGYYWSWIRQHPGKGLEWIGYIYYS |
| | | | | | | | | | GSTYYNPSLKSRVTISVDTSKNQFSLKLT |
| | | | | | | | | | SVTAADTAVYYCAREANDSGSFYNGPFDY |
| | | | | | | | | | WGQGTLVTVSS |
| COV2- | Ab | SARS-CoV1, | | | SARS-CoV2 | S; non- | B-cells; SARS-CoV2 | 482 | QVQLQESGPGLVKPSQTLSLTCTVSGGSI |
| 2536 | | SARS-CoV2 | | | | RBD | Human Patient | | SSGSYYWSWIRQPAGKGLEWIGRIFTSG |
| | | | | | | | | | STNYNPSLKSRVTISVDTSKNQFSLKLSS |
| | | | | | | | | | VTAADTAVYYCARGGLLWFGGAGNYMD |
| | | | | | | | | | VWGKGTTVTVSS |
| COV2- | Ab | SARS-CoV2 | SARS-CoV1 | SARS-CoV2 | | S; RBD | B-cells; SARS-CoV2 | 483 | QVQLAQSGAEVKKPGASVKVSCKAGGY |
| 2539 | | | | | | | Human Patient | | TFTSYDINWVRQATGQGLEWMGWMN |
| | | | | | | | | | SNSGNAGYAQKFQGRVTMTRDTSISTA |
| | | | | | | | | | YMELSSLRSEDTAVYYCARMRTGWPTH |
| | | | | | | | | | GRPDDFWGRGTLVTVSS |
| COV2- | Ab | SARS-CoV2 | SARS-CoV1 | | SARS-CoV1, | S; non- | B-cells; SARS-CoV2 | 484 | QVQIQQWGAGVLKPSETLSLTCAVYGG |
| 2545 | | | | | SARS-CoV2 | RBD | Human Patient | | SFSGHYWSWIRQPPGKGLEWIGEINHS |
| | | | | | | | | | GSTKYNPSLKSRVTISVDTSKNQFSLKLS |
| | | | | | | | | | SVTAADTAVYYCARGPPVTTFFVFSLLFD |
| | | | | | | | | | PWGQGTLVTVSS |
| COV2- | Ab | SARS-CoV2 | SARS-CoV1 | | SARS-CoV1, | S; non- | B-cells; SARS-CoV2 | 485 | QVQLVESGGGVVQPGRSLRLSCAASGF |
| 2546 | | | | | SARS-CoV2 | RBD | Human Patient | | TFSSYGMHWVRQAPGKGLEWVAVISY |
| | | | | | | | | | DGRNKYYADSVKGRFTISRDNSKNTLYL |
| | | | | | | | | | QMSSLRAEDTAVYYCAKEGEWELRGNA |
| | | | | | | | | | LDIWGQGTMVTVSS |
| COV2- | Ab | SARS-CoV2 | SARS-CoV1 | | SARS-CoV1, | S; non- | B-cells; SARS-CoV2 | 486 | QVQLVESGGGVVQPGRSLRLSCAASGF |
| 2549 | | | | | SARS-CoV2 | RBD | Human Patient | | TFSSYGMHWVRQAPDKGLEWVAVIWY |
| | | | | | | | | | DGSNKYYADSVKGRFTISRDNSKNTLYL |
| | | | | | | | | | QMNSLRAEDTAVYYCAREGQWPNQAF |
| | | | | | | | | | DIWGQGTMVTVSS |
| COV2- | Ab | SARS-CoV1, | | | SARS-CoV2 | S; non- | B-cells; SARS-CoV2 | 487 | QVQLQESGPGLVKPSETLSLTCTVSGGSI |
| 2551 | | SARS-CoV2 | | | | RBD | Human Patient | | SSSSYYWGWIRQPPGKGLEWIGSIYYSG |
| | | | | | | | | | SSYYSPSLKSRVTISADTSKNQFSLNLRS |
| | | | | | | | | | VTAADTAVYYCASGPPYMATFSYYFDYW |
| | | | | | | | | | GQGTLVTVSS |
| COV2- | Ab | SARS-CoV1, | | SARS-CoV2 | | S; RBD | B-cells; SARS-CoV2 | 488 | QVQLVESGGGLVKPGGSLRLSCAASGFT |
| 2552 | | SARS-CoV2 | | (weak) | | | Human Patient | | FSDYYMSWIRQAPGKGLECVSYISSSGST |
| | | | | | | | | | IYYADSVKGRFTISRDNAKNSLYLQMNSL |
| | | | | | | | | | RAEDTAVYYCARDPIRDGVWGLNENDY |
| | | | | | | | | | WGQGTLVTVSS |
| COV2- | Ab | SARS-CoV2 | SARS-CoV1 | | SARS-CoV1, | S; non- | B-cells; SARS-CoV2 | 489 | QVQLVESGGGVVQPGRSLRLSCAASGFI |
| 2553 | | | | | SARS-CoV2 | RBD | Human Patient | | FSSYAMHWVRQAPGKGLEWVAVISYD |
| | | | | | | | | | GSNKYYADSVKGRFTISRDNSKNTLYLQ |
| | | | | | | | | | MNSLRAEDTAVYYCARSPPASYYNPSTG |
| | | | | | | | | | YFDYWGQGTLVTVSS |
| COV2- | Ab | SARS-CoV1, | | | SARS-CoV2 | S; RBD | B-cells; SARS-CoV2 | 490 | QVQLVQSGAEVKKPGASVKVSCKASGY |
| 2554 | | SARS-CoV2 | | | | | Human Patient | | TFTSHYMHWVRQAPGQGLEWMGIINP |
| | | | | | | | | | SGGSTSYAQKFQGRVTMTSDTSTSTVY |
| | | | | | | | | | MELSSLRSEDTAMYYCARDVFWVPAAS |
| | | | | | | | | | SFDYWGQGTLVTVSS |
| COV2- | Ab | SARS-CoV2 | SARS-CoV1 | | SARS-CoV1, | S; NTD | B-cells; SARS-CoV2 | 491 | QVQLVESGGGVVQPGRSLRLSCAASGF |
| 2558 | | | | | SARS-CoV2 | | Human Patient | | TFSSYAMHWVRQAPGKGLEWVAVISY |
| | | | | | | | | | DGSNKYYADSVKGRFTISRDNSKNTLYL |
| | | | | | | | | | QMNSLRVEDTAVYYCARDQEWFRELFL |
| | | | | | | | | | FDYWGQGTLVTVSS |
| COV2- | Ab | SARS-CoV2 | SARS-CoV1 | SARS-CoV2 | | S; RBD | B-cells; SARS-CoV2 | 492 | QVQLAQSGAEVKKPGASVKVSCKAAGY |
| 2562 | | | | | | | Human Patient | | TFTSYDINWVRQATGQGLEWMGWMN |
| | | | | | | | | | SNSGNAGYAQKFQGRVTMTRDTSTSTA |
| | | | | | | | | | YMELSSLTSDDTAVYYCARMRTGWPTH |
| | | | | | | | | | GRPDDFWGRGTLVTVSS |
| COV2- | Ab | SARS-CoV2 | SARS-CoV1 | | SARS-CoV1, | S; NTD | B-cells; SARS-CoV2 | 493 | QVQLQQWGAGLLKPSETLSLTCAVYGG |
| 2563 | | | | | SARS-CoV2 | | Human Patient | | SFSGYYWSWIRQPPGKGLEWIGEINHS |
| | | | | | | | | | GSSNYNPSLKSRVTISVDTSKNQFSLKLS |
| | | | | | | | | | SVTAADTAVYYCARGWGWGAVAGRAEY |
| | | | | | | | | | YFDYWGQGTLVTVSS |
| COV2- | Ab | SARS-CoV1, | | | SARS-CoV2 | S; non- | B-cells; SARS-CoV2 | 494 | QVQLVESGGGVVQPGRSLRLSCAASGF |
| 2564 | | SARS-CoV2 | | | | RBD | Human Patient | | TFSSYAMHWVRQAPGKGLEWVAVISY |
| | | | | | | | | | DGYNKYYADSVKGRFTISRDNSKNTLYL |
| | | | | | | | | | QMNSLRAEDTAVYYCARAQGGNYYYG |
| | | | | | | | | | MDVWGQGTTVTVSS |
| COV2- | Ab | SARS-CoV1, | | | SARS-CoV2 | S; non- | B-cells; SARS-CoV2 | 495 | QVQLQESGPGLVKPSQTLSLTCTVSGGSI |
| 2565 | | SARS-CoV2 | | | | RBD | Human Patient | | SSGSYYWSWIRQPAGKGLEWIGRVYIYS |
| | | | | | | | | | SGSTNYNPSLKSRVTISVDTSKNQFSLKL |
| | | | | | | | | | SSVTAADTAVYYCARGAASFDYWGQGTL |
| | | | | | | | | | VTVSS |
| COV2- | Ab | SARS-CoV2 | SARS-CoV1 | | SARS-CoV1, | S; non- | B-cells; SARS-CoV2 | 496 | QLQLQESGPGLVKPSETLSLTCTVSGGSI |
| 2570 | | | | | SARS-CoV2 | RBD | Human Patient | | SSSSNYWGWIRQPPGKGLEWIGSIYYSG |
| | | | | | | | | | STYYNPSLKSRVTISVDTSKNQFSLKLSS |
| | | | | | | | | | VTAADTAVYYCARDPRVVVTARMYNWF |
| | | | | | | | | | DPWGQGTLVTVSS |
| COV2- | Ab | SARS-CoV2 | SARS-CoV1 | | SARS-CoV1, | S; NTD | B-cells; SARS-CoV2 | 497 | QVQLQESGPGLVKPSETLSLTCTVSGGSL |
| 2571 | | | | | SARS-CoV2 | | Human Patient | | SSYYWSWIRQPPGKGLEWIGYIYDSGG |
| | | | | | | | | | ASRSTNYNPSLKSRVTISVDTSRNQLSLK |
| | | | | | | | | | LSSVTAADTAVYYCARDQRQFQLLGRFG |
| | | | | | | | | | WFDPWGQGTLVTVSS |
| COV2- | Ab | SARS-CoV2 | SARS-CoV1 | | SARS-CoV1, | S; non- | B-cells; SARS-CoV2 | 498 | QLQLQESGPGLVKPSETLSLTCTVSGGSI |
| 2574 | | | | | SARS-CoV2 | RBD | Human Patient | | SSTTYYWGWIRQPPGKGLEWIASIYYSG |
| | | | | | | | | | STYYNPSLKSRLTVSVDTSKNQFSLKLSS |
| | | | | | | | | | VTAADTAVYYCARQWKWFGEAWYFDL |
| | | | | | | | | | WGRGTLVTVSS |
| COV2- | Ab | SARS-CoV2 | SARS-CoV1 | | SARS-CoV1, | S; non- | B-cells; SARS-CoV2 | 499 | QVQLVQSGSELKKPGASVKVSCKASGYT |
| 2582 | | | | | SARS-CoV2 | RBD | Human Patient | | FTTYAMNWVRQAPGQGLEWMGWIN |
| | | | | | | | | | TNTGNPTYAQGFTGRFVFSLDTSVNTAF |
| | | | | | | | | | LHIGSLKAEDTAVYYCARDQDSGYPTYYY |
| | | | | | | | | | YYMDVWGKGTTVTVSS |
| COV2- | Ab | SARS-CoV1, | | | SARS-CoV2 | S; non- | B-cells; SARS-CoV2 | 500 | QVQLVQSGAEVKKPGASVKVSCKASGY |
| 2583 | | SARS-CoV2 | | | | RBD | Human Patient | | TFTSYDMNWVRQATGQGLEWMGWM |
| | | | | | | | | | NPNSGNTGYAQKFQGRVTMTRNTSIST |
| | | | | | | | | | AYMELSSLRSEDTAVYYCARGGIYYLVRG |
| | | | | | | | | | FIIGYYGMDVWGQGTTVTVSS |
| COV2- | Ab | SARS-CoV2 | SARS-CoV1 | | SARS-CoV1, | S; non- | B-cells; SARS-CoV2 | 501 | EVALVESGGGLVKPGGSLRLSCAASGFIF |
| 2584 | | | | | SARS-CoV2 | RBD | Human Patient | | SNAWMTWVRQAPGKGLEWVGRIKSK |
| | | | | | | | | | SEGGTPEYAAPVKGRFIISRDDSTNSLHL |
| | | | | | | | | | QMNYLRIEDTAVYYCTTGGYSSYAASDY |
| | | | | | | | | | WGQGTLVTVSS |
| COV2- | Ab | SARS-CoV2 | SARS-CoV1 | | SARS-CoV1, | S; RBD | B-cells; SARS-CoV2 | 502 | EVQLVESGGGLVQPGGSLRLSCAASGFT |
| 2585 | | | | | SARS-CoV2 | | Human Patient | | FSSYSMNWVRQAPGKGLEWVSYISSRS |
| | | | | | | | | | STIKYADSVKGRFTISRDNAKNSLYLQM |
| | | | | | | | | | NSLRDEDTAVYYCARVDYYGSGSVYWYF |
| | | | | | | | | | DLWGRGTLVTVSS |
| COV2- | Ab | SARS-CoV2 | SARS-CoV1 | | SARS-CoV1, | S; non- | B-cells; SARS-CoV2 | 503 | EVQLVESGGGLVQPGRSLRLSCAASGFT |
| 2586 | | | | | SARS-CoV2 | RBD | Human Patient | | FDDYAMHWVRQAPGKGLEWVSGISW |
| | | | | | | | | | NSGSIAYADSVKGRFTISRDNAKNSLYLQ |
| | | | | | | | | | MNSLRAEDTALYFCAKVGWELSIDAFDL |
| | | | | | | | | | WGQGTMVTVSS |
| COV2- | Ab | SARS-CoV1, | | | SARS-CoV2 | S; non- | B-cells; SARS-CoV2 | 504 | QITFKESGPTLVKPTETLTLTCTFSGFSV |
| 2587 | | SARS-CoV2 | | | | RBD | Human Patient | | STSGEGVGWIRQPPGKALEWLAVIYWD |
| | | | | | | | | | DDKRYSPSLKSRLTITRDTSKNQVVLTMT |
| | | | | | | | | | NMDPVDTATYYCAHRLWFRDAFDIWG |
| | | | | | | | | | QGTTVTVSS |
| COV2- | Ab | SARS-CoV2 | SARS-CoV1 | SARS-CoV2 | | S; RBD | B-cells; SARS-CoV2 | 505 | QVTLRESGPALVKPTQTLTLTCTFSGFSL |
| 2589 | | | | (weak) | | | Human Patient | | STSGMCVSWIRQPPGKALEWLARIDWD |
| | | | | | | | | | DDKYYSTSLETRLTISKDTSKNQVVLTMT |
| | | | | | | | | | NMDPVDTATYYCARIQYQLNGMDVW |
| | | | | | | | | | GQGTTVTVSS |
| COV2- | Ab | SARS-CoV1, | | | SARS-CoV2 | S; non- | B-cells; SARS-CoV2 | 506 | QVQLVQSGAEVKKPGASVKVSCKASGY |
| 2590 | | SARS-CoV2 | | | | RBD | Human Patient | | TFSSYDINWVRQATGQGLEWMGWVN |
| | | | | | | | | | PNSGHTGYAQKFQGRVTMTRNTSVSTA |
| | | | | | | | | | YMELSSLRSEDTAVFYCARGRVGYVGSG |
| | | | | | | | | | SRGYYYYYDMDVWGQGTTVTVSS |
| COV2- | Ab | SARS-CoV1, | | | SARS-CoV2 | S; non- | B-cells; SARS-CoV2 | 507 | EVQLVQSGAEVKKPGESLKISCKGSGYSF |
| 2602 | | SARS-CoV2 | | | | RBD | Human Patient | | TSYWIGWVRQMPGKGLEWMGIIYPGD |
| | | | | | | | | | SDTRYSPSFQGQVTISADKSISTAYLQWS |
| | | | | | | | | | SLKASDTAMYYCARPDYSSGWFSYWYF |
| | | | | | | | | | DLWGRGTLVTVSS |
| COV2- | Ab | SARS-CoV1, | | | SARS-CoV2 | S; non- | B-cells; SARS-CoV2 | 508 | QVQLVQSGSALKKPGASVKVSCKASGYT |
| 2610 | | SARS-CoV2 | | | | RBD | Human Patient | | FTSYAMNWVRQAPGQGLEWMGWINT |
| | | | | | | | | | NTGNPTYAQGFTGRFVFSLDTSVSTAYL |
| | | | | | | | | | QISSLKAEDTAVYYCARGRSYGLSLGYW |
| | | | | | | | | | GQGTLVTVSS |
| COV2- | Ab | SARS-CoV2 | SARS-CoV1 | | SARS-CoV1, | S; non- | B-cells; SARS-CoV2 | 509 | QVQLVESGGGVVQPGRSLRVSCAASGF |
| 2611 | | | | | SARS-CoV2 | RBD | Human Patient | | TFSSHGMHWVRQAPGKGLEWVSVIWY |
| | | | | | | | | | DGSNKYYADSVKGRFTISRDNSKNTLSL |
| | | | | | | | | | QMNSLRAEDTAVYYCARESADISSRLDY |
| | | | | | | | | | WGRGTLVTVSS |
| COV2- | Ab | SARS-CoV1, | | | SARS-CoV2 | S; non- | B-cells; SARS-CoV2 | 510 | QITFKESGPTLVKPTETLTLTCTFSGFSV |
| 2614 | | SARS-CoV2 | | | | RBD | Human Patient | | STSGEGVGWIRQPPGKALEWLAVIYWD |
| | | | | | | | | | DDKRYSPSLKSRLTITRDTSKNQVVLTMT |
| | | | | | | | | | NMDPVDTATYYCAHRLWFRDAFDIWG |
| | | | | | | | | | QGTTVTVSS |
| COV2- | Ab | SARS-CoV1, | | | SARS-CoV2 | S; non- | B-cells; SARS-CoV2 | 511 | EVQLVESGGGLVQPGGSLRLSCAASGFI |
| 2616 | | SARS-CoV2 | | | | RBD | Human Patient | | LSDHYMDWVRQAPGKGLEWVGRTRN |
| | | | | | | | | | KANSYTTEYAASVKGRFTISRDDSKNSLY |
| | | | | | | | | | LQMNSLKTEDTAVYYCASVITFGGVIVRS |
| | | | | | | | | | YWGQGTLVTVSS |
| COV2- | Ab | SARS-CoV1, | | | SARS-CoV2 | S; non- | B-cells; SARS-CoV2 | 512 | QVQLVQSGAEVKKPGSSVKVSCKASGG |
| 2617 | | SARS-CoV2 | | | | RBD | Human Patient | | TFSSYAISWVRQAPGQGLEWMGGIIPV |
| | | | | | | | | | FGTTNYAQKLQGRVTISADESTSTAYME |
| | | | | | | | | | VSSLRSEDTAVYYCARVSGYGDYGAYSD |
| | | | | | | | | | YWGQGTLVTVSS |
| COV2- | Ab | SARS-CoV2 | SARS-CoV1 | | SARS-CoV1, | S; RBD | B-cells; SARS-CoV2 | 513 | EVQLVESGGGLVQPGGSLRLSCVASEFT |
| 2618 | | | | | SARS-CoV2 | | Human Patient | | FSSYWMSWFRQAPGKGLEWVSGINW |
| | | | | | | | | | NGGSTGYADSVKGRFTISRDNAKNSLYL |
| | | | | | | | | | QMNSLKTEDTAVYYCASVITFGGVIVRSY |
| | | | | | | | | | WGQGTLVTVSS |
| COV2- | Ab | SARS-CoV1, | | | SARS-CoV2 | S; non- | B-cells; SARS-CoV2 | 514 | EVQLLESGGGLVQPGGSLRLSCAASGFT |
| 2619 | | SARS-CoV2 | | | | RBD | Human Patient | | FNNYAMSWVRQAPGKGLEWVSAIGGS |
| | | | | | | | | | GGSTYYADSVKGRFTVSRDNSENTLYLQ |
| | | | | | | | | | MSSLRAEDTAVYYCARVEGDWLLGGPY |
| | | | | | | | | | YHYYGMDVWGQGTTVTVSS |
| COV2- | Ab | SARS-CoV1, | | | SARS-CoV2 | S; non- | B-cells; SARS-CoV2 | 515 | QVQLVESGGGVVQPGRSLRLSCAASGF |
| 2620 | | SARS-CoV2 | | | | RBD | Human Patient | | TFSSYAMHWVRQAPGKGLEWVAGISY |
| | | | | | | | | | DGSNKYYADSVKGRFTISRDNSKNTLYL |
| | | | | | | | | | QMNSLRAEDTAVYYCARADTMVRGTYF |
| | | | | | | | | | EYWGQGTLVTVSS |
| COV2- | Ab | SARS-CoV2 | SARS-CoV1 | | SARS-CoV1, | S; non- | B-cells; SARS-CoV2 | 516 | QVQLVESGGGVVQPGRSLRLSCAASGF |
| 2621 | | | | | SARS-CoV2 | RBD | Human Patient | | TFSSYLMHWVRQAPGKGLEWVAVIWA |
| | | | | | | | | | NGNRYYADSVKGRFTISRDISKNTLYLQ |
| | | | | | | | | | MNSLRAEDTAMYYCARDYCNGVTCNS |
| | | | | | | | | | NYWGQGTLVTVSS |
| COV2- | Ab | SARS-CoV2 | SARS-CoV1 | | SARS-CoV1, | S; NTD | B-cells; SARS-CoV2 | 517 | QVQLQESGPGLVKPSGTLSLTCAVSGGS |
| 2622 | | | | | SARS-CoV2 | | Human Patient | | ISSSNWWSWVRQPPGKGLEWIGEIYHS |
| | | | | | | | | | GSTNYNPSLKSRVTISVDKSKNQFSLKLS |
| | | | | | | | | | SVTAADTAVYYCARGWYFDYWGQGTLV |
| | | | | | | | | | TVSA |
| COV2- | Ab | SARS-CoV2 | SARS-CoV1 | | SARS-CoV1, | S; non- | B-cells; SARS-CoV2 | 518 | QVQLVQSGAEVKKPGSSVKVSCKASGG |
| 2624 | | | | | SARS-CoV2 | RBD | Human Patient | | TFHNYAISWVRQAPGQGLEWMGGFIPI |
| | | | | | | | | | LGTTNYAQKFQGRVTITADESTSTAYME |
| | | | | | | | | | LSSLRSEDTAVYYCARVEGEGVDSYYYG |
| | | | | | | | | | MDVWGQGTTVTVSS |
| COV2- | Ab | SARS-CoV2 | SARS-CoV1 | SARS-CoV2 | | S; RBD | B-cells; SARS-CoV2 | 519 | EVQLVESGGGLVQPGRSLRLSCAASGFT |
| 2628 | | | | (weak) | | | Human Patient | | FDDYAMHWVRQAPGKGLEWVSGISW |
| | | | | | | | | | NSGTIGYADSVKGRFIISRDNAKNSLYLQ |
| | | | | | | | | | MNSLRPEDTALYYCAKDIIRQGEDGMD |
| | | | | | | | | | VWGQGTTVTVSS |
| COV2- | Ab | SARS-CoV2 | SARS-CoV1 | | SARS-CoV1, | S; RBD | B-cells; SARS-CoV2 | 520 | QLQVVQSGPGLVKPSETLSLTCTVSGDP |
| 2631 | | | | | SARS-CoV2 | | Human Patient | | VINTNYYWGWIRQPPGKGLEWIGTLSY |
| | | | | | | | | | SGGTHYNPSLSSRVTIAVDSSKKRFSLTL |
| | | | | | | | | | RSVTAADTAIYYCARHPVDGYNYGYSDL |
| | | | | | | | | | WGQGTLVTVSS |
| COV2- | Ab | SARS-CoV2 | SARS-CoV1 | | SARS-CoV1, | S; RBD | B-cells; SARS-CoV2 | 521 | QVQLVQSGAEVKKPGASVKVSCKASGY |
| 2632 | | | | | SARS-CoV2 | | Human Patient | | TFTDYYMHWVRQAPGQGLEWMGWI |
| | | | | | | | | | NPNSRGTNYAQKFQGRVTMTRDTSIST |
| | | | | | | | | | VYMELSRLTSDDTAVYYCARVVVLGYGR |
| | | | | | | | | | PNNYYDGRNVWDYWGQGTLVTVSS |
| COV2- | Ab | SARS-CoV1, | | | SARS-CoV2 | S; non- | B-cells; SARS-CoV2 | 522 | QVQLVESGGGVVQPGRSLRLSCAASGF |
| 2639 | | SARS-CoV2 | | | | RBD | Human Patient | | TFSSYAMHWVRQAPGKGLEWVAVISY |
| | | | | | | | | | DEINKYYADSVKGRFTISRDNSKTTLDLQ |
| | | | | | | | | | MNSLRAEDTAVYYCARAGGGSYRGPFD |
| | | | | | | | | | YWGQGTLVTVSS |
| COV2- | Ab | SARS-CoV1, | | | SARS-CoV2 | S; non- | B-cells; SARS-CoV2 | 523 | QVQLVESGGGVVQPGRSLRLSCAASGF |
| 2641 | | SARS-CoV2 | | | | RBD | Human Patient | | TFSTYAMHWVRQAPGKGLEWVTVISYD |
| | | | | | | | | | GSNKYYADSVKGRFTISRDNSKNTLYLQ |
| | | | | | | | | | MNSLRAEDTAVYYCAKSYNGNYYDAFDI |
| | | | | | | | | | WGQGTMVTVSS |
| COV2- | Ab | SARS-CoV2 | SARS-CoV1 | | SARS-CoV1, | S; non- | B-cells; SARS-CoV2 | 524 | QVQLVESGGGVVQPGRSLRLSCAASGF |
| 2643 | | | | | SARS-CoV2 | RBD | Human Patient | | TFSSYGMHWVRQAPGKGLEWVAVISY |
| | | | | | | | | | DGSNKYYADSVKGRFTISRDNAKNSLYL |
| | | | | | | | | | QMNSLRAEDTAVYYCARGSAGNYYYG |
| | | | | | | | | | MDVWGQGTTVTVSS |
| COV2- | Ab | SARS-CoV2 | SARS-CoV1 | | SARS-CoV1, | S; non- | B-cells; SARS-CoV2 | 525 | EVQLVQSGAEVKKPGESLKISCKGSGYSF |
| 2656 | | | | | SARS-CoV2 | RBD | Human Patient | | SDYWIGWVRQMPGKGLEWMGIIYPG |
| | | | | | | | | | DSDTRYSPSFQGQVTISADKSISTAYLQW |
| | | | | | | | | | SSLKASDTAMYYCARLTFGGSGSYYFYYN |
| | | | | | | | | | GMDVWGQGTTVTVSS |
| COV2- | Ab | SARS-CoV2 | SARS-CoV1 | | SARS-CoV1, | S; non- | B-cells; SARS-CoV2 | 526 | EVQLVESGGGLVQPGGSLRLSCAASGFT |
| 2660 | | | | | SARS-CoV2 | RBD | Human Patient | | FSSYDMHWVRQATGKGLEWVSAIGTA |
| | | | | | | | | | GDTYYPGSVKGRFTISRENAKNSLYLQM |
| | | | | | | | | | NSLRAGDTAVYYCARADPYQLLGQHYYY |
| | | | | | | | | | GMDVWGQGTTVTVSS |
| COV2- | Ab | SARS-CoV1, | | | SARS-CoV2 | S; non- | B-cells; SARS-CoV2 | 527 | QVHLVESGGGVVQPGRSLRLSCAASGF |
| 2669 | | SARS-CoV2 | | | | RBD | Human Patient | | TFSNYGMHWVRQAPGKGLEWVAVISN |
| | | | | | | | | | DEFNKFYANSVKGRFTISRDNSKNTVYL |
| | | | | | | | | | QLNSLRTEDTARYYCAKGGDGSGWAW |
| | | | | | | | | | DGDNPPTDYWGQGTLVIVSS |
| COV2- | Ab | SARS-CoV2 | SARS-CoV1 | | SARS-CoV1, | S; NTD | B-cells; SARS-CoV2 | 528 | QVTLKESGPVLVKPTETLTLTCAVSGFSL |
| 2673 | | | | | SARS-CoV2 | | Human Patient | | SNAKMGVSWIRQPPGKALEWLAHIFSN |
| | | | | | | | | | DEKAYSTSLKTRLTISKDTSKSQVVLTVT |
| | | | | | | | | | NMDPVDTATYYCARIVLGASGTYPSPGFD |
| | | | | | | | | | PWGQGTLVTVSS |
| COV2- | Ab | SARS-CoV2 | SARS-CoV1 | | SARS-CoV1, | S; non- | B-cells; SARS-CoV2 | 529 | QVQLVESGGGVVQPGRSLRLSCAASGF |
| 2675 | | | | | SARS-CoV2 | RBD | Human Patient | | TFSIYGMHWVRQAPGKGLEWVAVISFD |
| | | | | | | | | | GSNKYYADSVKGRFTISRDNSKNTLYLQ |
| | | | | | | | | | MNSLRAEDTAVYYCAKDGSGSYYGWFD |
| | | | | | | | | | PWGQGTLVTVSS |
| COV2- | Ab | SARS-CoV2 | SARS-CoV1 | SARS-CoV2 | | S; non- | B-cells; SARS-CoV2 | 530 | QVQLVQSGAEVKKPGSSVKVSCKASGG |
| 2676 | | | | (weak) | | RBD | Human Patient | | TFSSYAINWVRQAPGQGLEWMGGIIPIF |
| | | | | | | | | | GTANYAQKFQGRVTFTADESTSTAYME |
| | | | | | | | | | VSSLRSEDTAVYYCARSCGDCYSADLDF |
| | | | | | | | | | WGQGTLVTVSS |
| COV2- | Ab | SARS-CoV1, | | SARS-CoV2 | | S; RBD | B-cells; SARS-CoV2 | 531 | QLQLQESGPGLVKPSETLSLTCTVSGGSI |
| 2677 | | SARS-CoV2 | | | | | Human Patient | | SSSSYYWGWIRQPPGKGLEWIGSMYYS |
| | | | | | | | | | GSTYYNPSLKSRVTISVDTSKNQFSLKLS |
| | | | | | | | | | SVTAADTAVYYCARLLWLRGHFDYWGQ |
| | | | | | | | | | GTLVTVSS |
| COV2- | Ab | SARS-CoV1, | | SARS-CoV2 | | S; RBD | B-cells; SARS-CoV2 | 532 | EVQLVESGGGVVRPGGSLRLSCAASGFI |
| 2678 | | SARS-CoV2 | | | | | Human Patient | | FDDYDMTWVRQAPGKGLEWVSGISW |
| | | | | | | | | | NGGNTGYADSVKGRFTISRDNAKNSLYL |
| | | | | | | | | | QMNSLRAEDTALYHCAVIMSPIPRYSGY |
| | | | | | | | | | DWAGGAFDIWGQGTMVTVSS |
| COV2- | Ab | SARS-CoV1, | | | SARS-CoV2 | S; non- | B-cells; SARS-CoV2 | 533 | QVQLVESGGGVVQPGRSLRLSCAASGF |
| 2681 | | SARS-CoV2 | | | | RBD | Human Patient | | TFSFYAIHWVRQAPGQGLEWAAAISSD |
| | | | | | | | | | GTYKYYADSVKGRFTISRDNSKNTSYLQ |
| | | | | | | | | | MNSLRAEDTAVYYCARALNKGFDPWG |
| | | | | | | | | | QGTLLTVSS |
| COV2- | Ab | SARS-CoV2 | SARS-CoV1 | SARS-CoV2 | | S; RBD | B-cells; SARS-CoV2 | 534 | QMQLVQSGPEVKKPGTSVKVSCKTSGF |
| 2684 | | | | (weak) | | | Human Patient | | TFTSSAIQWVRQARGQRLEWIGWIVVG |
| | | | | | | | | | SGNTNYAQKFQERVTITRDMSTSTAYM |
| | | | | | | | | | ELSSLRSEDTAVYYCAAPHCNRTSCYDAF |
| | | | | | | | | | DLWGQGTMVTVSS |
| COV2- | Ab | SARS-CoV1, | | | SARS-CoV2 | S; non- | B-cells; SARS-CoV2 | 535 | QVQLVQSGAEVKKPGASVQVSCEASGY |
| 2685 | | SARS-CoV2 | | | | RBD | Human Patient | | TFTTYYMHWVRQAPGQGLEWMGIINP |
| | | | | | | | | | SGGSTTYAQKFQGRVTMTRDTSTSTVY |
| | | | | | | | | | MDLSSLRSEDTAVYYCARDRLGDGSYLG |
| | | | | | | | | | GGYYGMDVWGQGTTVTVSS |
| COV2- | Ab | SARS-CoV2 | SARS-CoV1 | SARS-CoV2 | | S; RBD | B-cells; SARS-CoV2 | 536 | QVQLVQSGAEVKKPGASVKVSCQASGY |
| 2693 | | | | (weak) | | | Human Patient | | TFTSYDINWVRQATGQGLEWMGWMK |
| | | | | | | | | | SNSGNTGYAQKFQGRVTMTRNTSISTA |
| | | | | | | | | | YMELTSLRSEDTAVYYCARMRSGWPTH |
| | | | | | | | | | GRPDDLWGRGTLVTVSS |
| COV2- | Ab | SARS-CoV2 | SARS-CoV1 | | SARS-CoV1, | S; RBD | B-cells; SARS-CoV2 | 537 | QVQLVQSGAEVKRPGSSVKVSCKASGG |
| 2694 | | | | | SARS-CoV2 | | Human Patient | | TFSSYTISWVRQAPGQGLEWMGRIIPIL |
| | | | | | | | | | AVANYAQKFQGRVTITADKSTSTAYMEL |
| | | | | | | | | | SSLRSEDTAVYYCARDHSGYYDSTSLMSP |
| | | | | | | | | | FFDYWGQGTLVTVSS |
| COV2- | Ab | SARS-CoV2 | SARS-CoV1 | | SARS-CoV1, | S; non- | B-cells; SARS-CoV2 | 538 | QVQLVQSGAEVKKPGASVKVSCKASGY |
| 2697 | | | | | SARS-CoV2 | RBD | Human Patient | | TFTSHYMHWVRQAPGQGLEWMGIINP |
| | | | | | | | | | SGGSTSYAQKFQGRVTMTGDTSTSTVY |
| | | | | | | | | | MELSSLRSEDTAVYYCARDLAGVPAALG |
| | | | | | | | | | CWFDPWGQGTLVTVSS |
| COV2- | Ab | SARS-CoV2 | SARS-CoV1 | | SARS-CoV1, | S; non- | B-cells; SARS-CoV2 | 539 | QVQLVESGGGVVQPGRSLRLSCAASGF |
| 2700 | | | | | SARS-CoV2 | RBD | Human Patient | | TFSTYAMHWVRQAPGKGLEWVAVISY |
| | | | | | | | | | DGGNKYYADSVKGRFTISRDNSKNTLYL |
| | | | | | | | | | QMNSLRAEDTAVYYCAKNLGPYCSGGT |
| | | | | | | | | | CYSLVGDYWGQGTLVTVSS |
| COV2- | Ab | SARS-CoV2 | SARS-CoV1 | | SARS-CoV1, | S; RBD | B-cells; SARS-CoV2 | 540 | EVQLVESGGGLVQPGGSLRLSCAASGFT |
| 2703 | | | | | SARS-CoV2 | | Human Patient | | FSSYDMHWVRQATGKGLEWVSAIGTA |
| | | | | | | | | | GDTYYPGSVKGRFTISRENAKNSLYLQM |
| | | | | | | | | | NSLRAGDTAVYYCARARGGYNWNFDY |
| | | | | | | | | | WGQGTLVTVSS |
| COV2- | Ab | SARS-CoV1, | | | SARS-CoV2 | S; RBD | B-cells; SARS-CoV2 | 541 | QVQLVESGGGVVQPGRSLRLSCAASGF |
| 2705 | | SARS-CoV2 | | | | | Human Patient | | TFSSYAMHWVRQAPGKGLEWVAVISY |
| | | | | | | | | | DGSYKYFADSVKGRFTISRDNSKNTLYLQ |
| | | | | | | | | | MNSLRAEDTAVYYCARDQGTVVTHFDY |
| | | | | | | | | | WGQGTLVTVSS |
| COV2- | Ab | SARS-CoV1, | | | SARS-CoV2 | S; RBD | B-cells; SARS-CoV2 | 542 | QVQLVESGGGVVQPGRSLRLSCAASGF |
| 2709 | | SARS-CoV2 | | | | | Human Patient | | TFSNYGMHWVRQAPGKGLEWVAVMS |
| | | | | | | | | | YDGSNKYYADSVKGRFTISRDNSKNTLYL |
| | | | | | | | | | QMNSLRAEDTAVYYCAKNLGPYCSGGT |
| | | | | | | | | | CYSLVGDYWGQGTLVTVSS |
| COV2- | Ab | SARS-CoV2 | SARS-CoV1 | | SARS-CoV1, | S; RBD | B-cells; SARS-CoV2 | 543 | QVQLVQSGAEVKKPGASVKVSCKASGY |
| 2710 | | | | | SARS-CoV2 | | Human Patient | | TFTSYDINWVRQATGQGLEWMGWMS |
| | | | | | | | | | PNSGNTGYAQKFQGRVTMTRDTSISTA |
| | | | | | | | | | YMELNSLRSEDTAVYYCARMRSGWPTH |
| | | | | | | | | | GRPDDHWGQGTLVTVSS |
| COV2- | Ab | SARS-CoV1, | | | SARS-CoV2 | S; RBD | B-cells; SARS-CoV2 | 544 | EVQLVESGGGVVQPGGSLRLSCAASGF |
| 2713 | | SARS-CoV2 | | | | | Human Patient | | TFDDYAMHWVRQAPGKGLEWVSLISG |
| | | | | | | | | | DGGNTYYADSVKGRFTISRDNSKNSLYL |
| | | | | | | | | | QMNSLRTEDTALYYCAKDEMAYPPSHH |
| | | | | | | | | | YYYYYMDVWGKGTTVTVSS |
| COV2- | Ab | SARS-CoV1, | | | SARS-CoV2 | S; RBD | B-cells; SARS-CoV2 | 545 | QVQLVQSGAEVKTPGASVKVSCKASGY |
| 2717 | | SARS-CoV2 | | | | | Human Patient | | TFTSYDINWVRQATGQGPEWMGWM |
| | | | | | | | | | NPNSGNTGYAHKFQGRVTMTRNTSIST |
| | | | | | | | | | AYMELSSLRSEDTAVYYCARGPSILTGFY |
| | | | | | | | | | NPLDYWGQGTLVTVSS |
| COV2- | Ab | SARS-CoV2 | SARS-CoV1 | | SARS-CoV1, | S; non- | B-cells; SARS-CoV2 | 546 | QLQLQESGPGLVKPSETLSLTCTVSGGSI |
| 2718 | | | | | SARS-CoV2 | RBD | Human Patient | | SSSNYYWGWIRQPPGKGLEWIGTIHYS |
| | | | | | | | | | GISYYNPSLKSRVTISVDTSNNKFSLELS |
| | | | | | | | | | SVTAADTAVYFCARRTYYDLWSAYSSTAY |
| | | | | | | | | | YCMDVWGKGTTVTVSS |
| COV2- | Ab | SARS-CoV1, | | SARS-CoV2 | | S; RBD | B-cells; SARS-CoV2 | 547 | QVRLVQSGAEVKKPGSSVKVSCKASGG |
| 2722 | | SARS-CoV2 | | | | | Human Patient | | TFSDYAISWVRQAPGQGLEWMGGIIPIF |
| | | | | | | | | | GTANYAQKFQGRVTITADEFTITAYMEL |
| | | | | | | | | | SSLRSEDTAVYYCARLSGSGWLGYAMD |
| | | | | | | | | | VWGQGTTVTVSS |
| COV2- | Ab | SARS-CoV2 | SARS-CoV1 | | SARS-CoV1, | S; non- | B-cells; SARS-CoV2 | 548 | QVQLVESGGGVVQPGRSLRLSCAASGF |
| 2726 | | | | | SARS-CoV2 | RBD | Human Patient | | TFSSYAMHWVRQAPGKGLEWVAVISN |
| | | | | | | | | | DGRNKYYADSVKGRLTISRDNSKNTLYL |
| | | | | | | | | | QMNSLRAEDTAVYYCARPSNWYFDLW |
| | | | | | | | | | GRGTLVTVSS |
| COV2- | Ab | SARS-CoV2 | SARS-CoV1 | SARS-CoV2 | | S; RBD | B-cells; SARS-CoV2 | 549 | EVQLVESGGGLVQPGRSLTLSCAASGFT |
| 2730 | | | | (weak) | | | Human Patient | | FDDYTMHWVRQAPGKGLEWVSGIDW |
| | | | | | | | | | NGGTIGYADSVKGRFTISRDNAKNSLYL |
| | | | | | | | | | QMNSLRAEDTALYYCAKAGYYAYVWGS |
| | | | | | | | | | YRFEYFDNWGQGTLVTVSS |
| COV2- | Ab | SARS-CoV2 | SARS-CoV1 | SARS-CoV2 | | S; RBD | B-cells; SARS-CoV2 | 550 | EVQLVESGGGLVQPGGSLRLSCAASGVT |
| 2733 | | | | | | | Human Patient | | VSSNYMSWVRQAPGKGLEWVSLIYSGG |
| | | | | | | | | | STFYADSVKGRFTISRHNSKNTLYLQMN |
| | | | | | | | | | SLRPEDTAVYYCARGPEPDAFDIWGQG |
| | | | | | | | | | TMVTVSS |
| COV2- | Ab | SARS-CoV1, | | SARS-CoV2 | | S; RBD | B-cells; SARS-CoV2 | 551 | QVQLQESGPGLVKPSETLSLTCTVSGGS |
| 2734 | | SARS-CoV2 | | (weak) | | | Human Patient | | VSSGSYYWSWIRQPPGKGLECIGYIYYSG |
| | | | | | | | | | SSNYNPSLKSRVTISVDTSKNQFSLKMSS |
| | | | | | | | | | VTAADTAVYYCAGSPVPPTIVGASYWG |
| | | | | | | | | | QGTLVTVSS |
| COV2- | Ab | SARS-CoV1, | | | SARS-CoV2 | S; RBD | B-cells; SARS-CoV2 | 552 | QVQLVESGGGVVQPGRSLRLSCAASGF |
| 2736 | | SARS-CoV2 | | | | | Human Patient | | TFSDYAMHWVRQAPGKGLEWVADISF |
| | | | | | | | | | DGSNKYYADSVKGRFTISRDSSENTLYLQ |
| | | | | | | | | | MDSLRADDTAVYYCARDLSTTWYLEM |
| | | | | | | | | | WGPDAFDIWGQGTVVTVSS |
| COV2- | Ab | SARS-CoV1, | | | SARS-CoV2 | S; RBD | B-cells; SARS-CoV2 | 553 | QVQLVESGGGVVQPGRSLRLSCAASGF |
| 2740 | | SARS-CoV2 | | | | | Human Patient | | TFRRYGMYWVRQAPGKGLEWVAVISY |
| | | | | | | | | | DGTDKYYTDSVKGRFTISRDNSKNTLYLQ |
| | | | | | | | | | MNSLRAEDTAVYYCAKKGGPYCGGGN |
| | | | | | | | | | CYAGYFDYWGQGTLVTVSS |
| COV2- | Ab | SARS-CoV1, | | | SARS-CoV2 | S; RBD | B-cells; SARS-CoV2 | 554 | QVQLQQWGAGLLKPSETLSLTCAVSGG |
| 2749 | | SARS-CoV2 | | | | | Human Patient | | SFSAYYWSWIRQPPGKGLEWIGEINHS |
| | | | | | | | | | GSTNYNPSLRSRVTISVDTSKNQFSLKLS |
| | | | | | | | | | SVTAADTAVYYCARVGYSQGYYYYYMDV |
| | | | | | | | | | WGKGTTVTVSS |
| COV2- | Ab | SARS-CoV2 | SARS-CoV1 | | SARS-CoV1, | S; RBD | B-cells; SARS-CoV2 | 555 | EVQVVESGGGLVQPGRSLRLSCVSSGFI |
| 2751 | | | | | SARS-CoV2 | | Human Patient | | FDDYVMHWVRQRPGKGLEWVAGITYN |
| | | | | | | | | | GGILGYGDSVKGRFIIARDNVRGFLSLQ |
| | | | | | | | | | MGDLRTEDTALYYCARDYCSSTTCPAET |
| | | | | | | | | | YYYMDVWGKGTAVTVSS |
| COV2- | Ab | SARS-CoV2 | SARS-CoV1 | SARS-CoV2 | | S; RBD | B-cells; SARS-CoV2 | 556 | EVQLVESGGGLIQPGGSLRLSCAASEVT |
| 2752 | | | | | | | Human Patient | | VSSNYMSWVRQAPGKGLEWVSLIYSGG |
| | | | | | | | | | TTYYADSVKGRFTISRDNSKNTLYLQMN |
| | | | | | | | | | SLRAEDTAVYYCARDFLRWHDLWGQG |
| | | | | | | | | | TLVTVSS |
| COV2- | Ab | SARS-CoV1, | | | SARS-CoV2 | S; RBD | B-cells; SARS-CoV2 | 557 | EVQLVESGGGLVQPGGSLRLSCAASGFT |
| 2753 | | SARS-CoV2 | | | | | Human Patient | | FSSYDMHWVRQATGKGLEWVSTIGTA |
| | | | | | | | | | GDTYYPDSVKGRFTISRENAKNSLFLQM |
| | | | | | | | | | NSLRAGDTAVYYCARVDFDILTGYYSNW |
| | | | | | | | | | GQGTLVTVSS |
| COV2- | Ab | SARS-CoV2 | SARS-CoV1 | | SARS-CoV1, | S; RBD | B-cells; SARS-CoV2 | 558 | EAQLLESGGALVQPGGSLRLSCAASGFT |
| 2756 | | | | | SARS-CoV2 | | Human Patient | | FSCCAMGWVRQAPGRGLEWVSSIHDD |
| | | | | | | | | | GVGTFYAVSVKGRFSISRDNSKNTVYLQ |
| | | | | | | | | | MNGLRAEDTGVYYCAKWAGPIVMKYY |
| | | | | | | | | | LQYWGQGALVTVSS |
| COV2- | Ab | SARS-CoV1, | | | SARS-CoV2 | S; RBD | B-cells; SARS-CoV2 | 559 | EVQLVESGGGLVQPGGSLRLSCAASGFT |
| 2758 | | SARS-CoV2 | | | | | Human Patient | | FSSYDMHWVRQATGKGLEWVSAIGTA |
| | | | | | | | | | GDTYYPDSVKGRFTISRENAKNSLYLQM |
| | | | | | | | | | NSLRAGDTAVYYCARGGDSGYDLGAWY |
| | | | | | | | | | FDLWGRGTLVTVSS |
| COV2- | Ab | SARS-CoV2 | SARS-CoV1 | | SARS-CoV1, | S; RBD | B-cells; SARS-CoV2 | 560 | EVQLVQSGAEVKKPGESLKISCKGSGYSF |
| 2759 | | | | | SARS-CoV2 | | Human Patient | | TNYWIGWVRQMPGKGLEWMGIIYPG |
| | | | | | | | | | DSDTRYSPSFQGQVTISADKSISTAYLQW |
| | | | | | | | | | SSLKASDTAMYYCARTPTLYNWFHPWG |
| | | | | | | | | | QGTPVTVSS |
| COV2- | Ab | SARS-CoV2 | SARS-CoV1 | SARS-CoV2 | | S; RBD | B-cells; SARS-CoV2 | 561 | QVTLRESGPALVKPTQTLTLTCTFSGFSL |
| 2760 | | | | (weak) | | | Human Patient | | STSGLCVSWIRQPPGKALEWLARIDWDD |
| | | | | | | | | | DKYYNTSLRTRLTISKDTSKNQVVLTMTN |
| | | | | | | | | | MDPVDTATYYCARATTFFYGMDVWGQ |
| | | | | | | | | | GTTVTVSS |
| COV2- | Ab | SARS-CoV2 | SARS-CoV1 | | SARS-CoV1, | S; RBD | B-cells; SARS-CoV2 | 562 | QLQLQESGPGLVKPSETLSLTCTVSGGSI |
| 2762 | | | | | SARS-CoV2 | | Human Patient | | SSSSYYWGWIRQPPGKGLEWIGSIYYSG |
| | | | | | | | | | ITYYNPSLKSRVTISVDTSKNQFSLKLSS |
| | | | | | | | | | VTAADTAVYYCARHQRYCSSSSCHVWDYW |
| | | | | | | | | | GQGTLVTVSS |
| COV2- | Ab | SARS-CoV1, | | | SARS-CoV2 | S; RBD | B-cells; SARS-CoV2 | 563 | EVQLVESGGGLVQPGRSLRLSCAASGFT |
| 2765 | | SARS-CoV2 | | | | | Human Patient | | FDDYAMNWVRQPPGKGLEWVSGISW |
| | | | | | | | | | NSDSIGYADSVKGRFTISRDNAKNSLYLQ |
| | | | | | | | | | MNSLRAEDTAMYYCAKGRGAGYTSYM |
| | | | | | | | | | DVWGKGTTVTVSS |
| COV2- | Ab | SARS-CoV2 | SARS-CoV1 | | SARS-CoV1, | S; RBD | B-cells; SARS-CoV2 | 564 | EVQLVESGGGLVQPGGSLRLSCAASGFT |
| 2767 | | | | | SARS-CoV2 | | Human Patient | | VSSNYMSWVRQAPGKGLEWVSVIYPG |
| | | | | | | | | | GSAFYADSVKGRFTISRHNSNNTLCLQM |
| | | | | | | | | | NSLRTEDTAVYYCARSYDILTGYRDAFDI |
| | | | | | | | | | WGQGTMVTVSS |
| COV2- | Ab | SARS-CoV2 | SARS-CoV1 | | SARS-CoV1, | S; RBD | B-cells; SARS-CoV2 | 565 | QVQLVESGGGVVQPGRSLRLSCAASGF |
| 2768 | | | | | SARS-CoV2 | | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARDDNSPQGSG |
| | | | | | | | | | WYFYYYYAMDVWGQGTTVTVSS |
| COV2- | Ab | SARS-CoV2 | SARS-CoV1 | | SARS-CoV1, | S; NTD | B-cells; SARS-CoV2 | 566 | QVQLVESGGGVVQPGRSLRLSCAASGF |
| 2769 | | | | | SARS-CoV2 | | Human Patient | | TFSTYAMHWVRQAPGKGLEWVAVISY |
| | | | | | | | | | DGSNKYYADSVKGRFTISRDNSKNTLYL |
| | | | | | | | | | QMIGLRAEDTAVYYCARDWAPTYYDM |
| | | | | | | | | | PSAFDIWGQGTMVTVSS |
| COV2- | Ab | SARS-CoV1, | | | SARS-CoV2 | S; non- | B-cells; SARS-CoV2 | 567 | EVQLVESGGGLVQPGGSLRLSCAASGFT |
| 2774 | | SARS-CoV2 | | | | RBD | Human Patient | | FSSYWMTWVRQAPGKGLEWVANIKQ |
| | | | | | | | | | DGSEKYYVDSVKGRFTISRDNAKNSLSL |
| | | | | | | | | | QMNSLRVEDTAVYYCVRLGVSSWYFDY |
| | | | | | | | | | WGQGTLVTVSS |
| COV2- | Ab | SARS-CoV1, | | | SARS-CoV2 | S; non- | B-cells; SARS-CoV2 | 568 | EVQLVESGGDLVQPGGSLRLTCAASGFT |
| 2776 | | SARS-CoV2 | | | | RBD | Human Patient | | FSSHWMTWVRQAPGKGLEWVANIKE |
| | | | | | | | | | DGREKYYVDSVKGRLTISRDNAKNSLYL |
| | | | | | | | | | QMNSLRAEDTAVYYCARVVVEVATNKG |
| | | | | | | | | | IHGVDYYYYYYMDVWGKGTTVTVSS |
| COV2- | Ab | SARS-CoV2 | SARS-CoV1 | SARS-CoV2 | | S; RBD | B-cells; SARS-CoV2 | 569 | QVQLQQWGAGLLKPSETLSRTCAVYGA |
| 2780 | | | | (weak) | | | Human Patient | | SFSNYYWSWIRQPPGKGLEWIGEINHSE |
| | | | | | | | | | NTNYNPSLKSRVTISVDTSKNQFSLRLSS |
| | | | | | | | | | VTAADTAVYYCARLRYSSSGGHIFDYWG |
| | | | | | | | | | QGTLVTVSS |
| COV2- | Ab | SARS-CoV1, | | | SARS-CoV2 | S; RBD | B-cells; SARS-CoV2 | 570 | QVQLVQSGAEVKKPGSSVKVSCKASGG |
| 2783 | | SARS-CoV2 | | | | | Human Patient | | TFSSYAISWVRQAPGQGLEWMGGIIPIF |
| | | | | | | | | | GTANYAQKFQGRVTITADESTSTAYMEL |
| | | | | | | | | | SSLRSEDTAVYYCARGLTGSSAYKDEIYF |
| | | | | | | | | | DYWGQGTLVTVSS |
| COV2- | Ab | SARS-CoV2 | SARS-CoV1 | | SARS-CoV1, | S; non- | B-cells; SARS-CoV2 | 571 | QVQLQESGPGLVKPSETLSLTCTVSGGSI |
| 2784 | | | | | SARS-CoV2 | RBD | Human Patient | | SSYYWSWIRQPPGKGLEWIGYIYYSGST |
| | | | | | | | | | KYNPSLKSRVTISVDTSKNQFSLKLSSVT |
| | | | | | | | | | AADTAVYYCARDGGNAYSSGWYRYYYH |
| | | | | | | | | | MDVWGKGTTVTVSS |
| COV2- | Ab | SARS-CoV2 | SARS-CoV1 | | SARS-CoV1, | S; RBD | B-cells; SARS-CoV2 | 572 | EVQLVESGGGLVQPGGSLRLSCAASGFT |
| 2786 | | | | | SARS-CoV2 | | Human Patient | | FSSYDLHWVRQGTGKRLEWVSAIGTAG |
| | | | | | | | | | DTYYLGSVKGRFTISRENAKNSLYLQMN |
| | | | | | | | | | SLRAGDTAVYYCARVLYDSSGFYNWFDP |
| | | | | | | | | | WGQGTLVTVSS |
| COV2- | Ab | SARS-CoV2 | SARS-CoV1 | | SARS-CoV1, | S; RBD | B-cells; SARS-CoV2 | 573 | QVQLVQSGAEVKKPGASVRVSCKAPGY |
| 2789 | | | | | SARS-CoV2 | | Human Patient | | TFTTYYIHWVRQAPGQGLEWMGIINPS |
| | | | | | | | | | AGSTTYAQKFQGRVTMTRDTSTSTVYM |
| | | | | | | | | | ELSSLRSEDTAVYYCARGFHVPAALRNW |
| | | | | | | | | | FDPWGQGTLVTVSS |
| COV2- | Ab | SARS-CoV2 | SARS-CoV1 | SARS-CoV2 | | S; RBD | B-cells; SARS-CoV2 | 574 | EVQLVESGGGLVQPGGSLRLSCAASGFT |
| 2790 | | | | | | | Human Patient | | VSSNYMSWVRQAPGKGLEWVSVIYPG |
| | | | | | | | | | GSAFYADSVKGRFTISRHNSNNTLCLQM |
| | | | | | | | | | NSLRTEDTAVYYCARSYDILTGYRDAFDI |
| | | | | | | | | | WGQGTMVTVSS |
| COV2- | Ab | SARS-CoV1, | | | SARS-CoV2 | S; RBD | B-cells; SARS-CoV2 | 575 | EAQLVESGGGLVQPGGSLRLSCEASGFI |
| 2794 | | SARS-CoV2 | | | | | Human Patient | | FSSYWMSWVRQAPGKGLEWVANIKQ |
| | | | | | | | | | DGSEKYYVDSVKGRFTISRDNAKNSLYL |
| | | | | | | | | | QMNSLRAEDTAVYYCARVNDGRPNPLE |
| | | | | | | | | | YYFDYWGQGTLVTVSS |
| COV2- | Ab | SARS-CoV2 | SARS-CoV1 | | SARS-CoV1, | S; RBD | B-cells; SARS-CoV2 | 576 | QVQLQESGPGLVKPSQTLSLTCTVSGDSI |
| 2796 | | | | | SARS-CoV2 | | Human Patient | | SSGRYYWSWIRQHPGKGLEWIGFVYYS |
| | | | | | | | | | GSTYYNPSLKSRVTISVDTSKNQFSLRLS |
| | | | | | | | | | SVTAADTAVYYCARETYSAYEMPPYFDY |
| | | | | | | | | | WGQGTLVTVSS |
| COV2- | Ab | SARS-CoV1, | | | SARS-CoV2 | S; RBD | B-cells; SARS-CoV2 | 577 | EVQLVQSGAEVKKPGESLKISCKGSGYSF |
| 2797 | | SARS-CoV2 | | | | | Human Patient | | TSYWIGWVRQMPGKGLEWMGIIQPG |
| | | | | | | | | | DSDTRYSPSFQGQVTMSADKSTSTAYLQ |
| | | | | | | | | | WSSLKASDTAMYYCARDLIIESTIAARPG |
| | | | | | | | | | YYGMDVWGQGTTVTVSS |
| COV2- | Ab | SARS-CoV2 | SARS-CoV1 | | SARS-CoV1, | S; RBD | B-cells; SARS-CoV2 | 578 | QVHLVQSGAEVKKPGASVKLSCKASGYT |
| 2801 | | | | | SARS-CoV2 | | Human Patient | | FTNYLLHWVRQAPGQGLEWMGNVNP |
| | | | | | | | | | SRGTATYPQKLDDRVTMTSDKSASTIYM |
| | | | | | | | | | ELSGLRSEDTAIYYCARERSGTYFFDYWG |
| | | | | | | | | | QGTLLTVSS |
| COV2- | Ab | SARS-CoV2 | SARS-CoV1 | SARS-CoV2 | | S; RBD | B-cells; SARS-CoV2 | 579 | QVTLRESGPALVKPTQTLTLTCTFSGFSL |
| 2807 | | | | | | | Human Patient | | TTSGMCVSWIRQPPGKALEWLARIDWD |
| | | | | | | | | | DDKYYSTSLQTRLTISKDTSKNQVVLTMT |
| | | | | | | | | | NMDPVDTATYYCARETPVTAIDYWGQ |
| | | | | | | | | | GTLVTVSS |
| COV2- | Ab | SARS-CoV1, | | | SARS-CoV2 | S; RBD | B-cells; SARS-CoV2 | 580 | QVQLVQSGAEVKKPGSSVKVSCKASGFT |
| 2808 | | SARS-CoV2 | | | | | Human Patient | | FMSSAVQWVRQARGQRLEWIGWIVIG |
| | | | | | | | | | SGNTNYAQKFQERVTITRDMSTSTAYM |
| | | | | | | | | | ELSSLRSEDTAVYYCAAPYCSSISCNDGF |
| | | | | | | | | | DIWGQGTMVTVSS |
| COV2- | Ab | SARS-CoV2 | SARS-CoV1 | | SARS-CoV1, | S; RBD | B-cells; SARS-CoV2 | 581 | QVQLVQSGAEVKKPGASVKVSCKVSGY |
| 2809 | | | | | SARS-CoV2 | | Human Patient | | TFTGYVVHWVRQAPGQDLEWMGWIN |
| | | | | | | | | | TGYGNTKYSQKFQGRVTISWDTSATTAY |
| | | | | | | | | | MELSNLKSEDKAVYYCASMTRMSEQTY |
| | | | | | | | | | YGMDVWGQGTTVTVSS |
| COV2- | Ab | SARS-CoV1, | | | SARS-CoV2 | S; non- | B-cells; SARS-CoV2 | 582 | QVQLVESGGGVVQPGRSLRLSCAASGF |
| 2811 | | SARS-CoV2 | | | | RBD | Human Patient | | TFSSFAMHWVRQAPGKGLEWVAVISY |
| | | | | | | | | | DGSNKYYADSVKGRFTISRDNSKNTLYL |
| | | | | | | | | | QMNSLRAEDTAAYYCARELMSVGWGQ |
| | | | | | | | | | GTLVTVSS |
| COV2- | Ab | SARS-CoV2 | SARS-CoV1 | SARS-CoV2 | | S; RBD | B-cells; SARS-CoV2 | 583 | EVQLVESGGGLIQPGGSLRLSCAASGITV |
| 2812 | | | | | | | Human Patient | | SSNYMSWVRQAPGKGLEWVSVIYAGG |
| | | | | | | | | | STYYADSVKGRFTISRDNSKNTLYLQMN |
| | | | | | | | | | SLRAEDTAVYYCARDALYYNGPGRDGM |
| | | | | | | | | | DVWGQGTTVTVSS |
| COV2- | Ab | SARS-CoV2 | SARS-CoV1 | SARS-CoV2 | | S; RBD | B-cells; SARS-CoV2 | 584 | EVQLVESGGGLIQPGGSLRLSCAASGITV |
| 2813 | | | | | | | Human Patient | | SSNYMSWVRQAPGKGLEWVSVIYAGG |
| | | | | | | | | | STYYADSVKGRFTISRDNSKNTLYLQMN |
| | | | | | | | | | SLRAEDTAVYYCARDALYYNGPGRDGM |
| | | | | | | | | | DVWGQGTTVTVSS |
| COV2- | Ab | SARS-CoV1, | | | SARS-CoV2 | S; non- | B-cells; SARS-CoV2 | 585 | QVQLVESGGGVVQPGRSLRLSCAASGF |
| 2814 | | SARS-CoV2 | | | | RBD | Human Patient | | TFSSYGMHWVRQAPGKGLEWVAVISS |
| | | | | | | | | | DGSNKYYAGSVKGRFTISRDNSKNTLYL |
| | | | | | | | | | QMNSLRAEDTAVYYCAKDMVEPLFSHY |
| | | | | | | | | | YYYGMDVWGQGTTVTVSS |
| COV2- | Ab | SARS-CoV2 | SARS-CoV1 | | SARS-CoV1, | S; NTD | B-cells; SARS-CoV2 | 586 | QVQLQESGPGLVKPSETLSLTCTVSGGSI |
| 2816 | | | | | SARS-CoV2 | | Human Patient | | SSYYWSWIRQPPGKGLEWIGYIYYSGST |
| | | | | | | | | | NYNPSLKSRVTISVDMSKNQFSLKLRSVT |
| | | | | | | | | | AADTAVYYCARAPRERLQWGEYYFDYW |
| | | | | | | | | | GQGTLVTVSS |
| COV2- | Ab | SARS-CoV1, | | | SARS-CoV2 | S; RBD | B-cells; SARS-CoV2 | 587 | EVQLVESGGGLVQPGGSLRLSCAASGFT |
| 2817 | | SARS-CoV2 | | | | | Human Patient | | FRSYDMHWVRQVTGKGLEWVSTIGTA |
| | | | | | | | | | GDTYYPGSVKGRFTISRENAKNSLYLQM |
| | | | | | | | | | NSLRAGDTAVYYCARVFETKVIRGGRYY |
| | | | | | | | | | YYYYMDVWGKGTTVTVSS |
| COV2- | Ab | SARS-CoV2 | SARS-CoV1 | | SARS-CoV1, | S; non- | B-cells; SARS-CoV2 | 588 | QVQLQQWGAGLLKPSETLSLTCAVYGG |
| 2818 | | | | | SARS-CoV2 | RBD | Human Patient | | SFSGYYWSWIRQPPGKGLEWIGEINHS |
| | | | | | | | | | GSTNYNPSLKSRVTISVDTSKNHFSLKM |
| | | | | | | | | | NSVTAADTAVYYCARCRQMGNFYYYYM |
| | | | | | | | | | DVWGKGTTVTVSS |
| COV2- | Ab | SARS-CoV2 | SARS-CoV1 | SARS-CoV2 | | S; RBD | B-cells; SARS-CoV2 | 589 | QVQLQESGPGLVKPSQTLSLTCTVSGDSI |
| 2819 | | | | | | | Human Patient | | SSAGYYWSWIRQHPGKGLEWIGYIYYSG |
| | | | | | | | | | RTYYNPSLKSRVTMSVDTSKNQFSLRLRS |
| | | | | | | | | | VTAADTAVYYCARVVPTRGPVAWFDP |
| | | | | | | | | | WGQGTLVTVSS |
| COV2- | Ab | SARS-CoV1, | | | SARS-CoV2 | S; non- | B-cells; SARS-CoV2 | 590 | QVQLVESGGGVVQPGRSLRLSCAASGF |
| 2820 | | SARS-CoV2 | | | | RBD | Human Patient | | TFSSYGMHWVRQAPGKGLEWVAVIWY |
| | | | | | | | | | DGSKKDYADSVKGRFTISRDNSKNTLYL |
| | | | | | | | | | QMNSLRAEDTAVYYCARDQSQGAYILT |
| | | | | | | | | | GYRGYGMDVWGQGTTVTVSS |
| COV2- | Ab | SARS-CoV1, | | | SARS-CoV2 | S; RBD | B-cells; SARS-CoV2 | 591 | QVQLVQSGAEVKKPGSSVKVSCKTAGG |
| 2821 | | SARS-CoV2 | | | | | Human Patient | | TSSSYAISWVRQAPGQGLEWMGRIIPIL |
| | | | | | | | | | GVAIYAQKFQGRVTITADKSTSTAYMEL |
| | | | | | | | | | NSLRSEDTAVYYCTTTQGGDYGDNLYYL |
| | | | | | | | | | DYWGQGTLVTVSS |
| COV2- | Ab | SARS-CoV2 | SARS-CoV1 | | SARS-CoV1, | S; RBD | B-cells; SARS-CoV2 | 592 | EVQLVESGGGLVQPGGSLRLSCAASGVT |
| 2822 | | | | | SARS-CoV2 | | Human Patient | | VSSNYMSWVRQAPGKGLEWVSLIYSGG |
| | | | | | | | | | STFYADSVKGRFTISRHNSKNTLYLQMN |
| | | | | | | | | | SLRPEDTAVYYCARGPEPDAFDIWGQG |
| | | | | | | | | | TMVTVSS |
| COV2- | Ab | SARS-CoV2 | SARS-CoV1 | | SARS-CoV1, | S; RBD | B-cells; SARS-CoV2 | 593 | QVQLVQSGAEVKKPGASVKVSCKASGD |
| 2826 | | | | | SARS-CoV2 | | Human Patient | | TFTTYYIHWVRQAPGQGLEWMGIINPS |
| | | | | | | | | | GGSRSYAQKFQGRISMTSDTSTSTVYME |
| | | | | | | | | | LSSLRSEDTAVYYCARGYGFVPNVLYYFD |
| | | | | | | | | | YWGQGTLVTVST |
| COV2- | Ab | SARS-CoV1, | | SARS-CoV2 | | S; RBD | B-cells; SARS-CoV2 | 594 | EVQLVESGGGLVKPGGSLRLSCAASGFT |
| 2828 | | SARS-CoV2 | | | | | Human Patient | | FRDYSMNWVRQAPGKGLEWVSSISSG |
| | | | | | | | | | GSYIYYADSVKGRFTISRDNAKNSLYLQM |
| | | | | | | | | | NSLRAEDTAVYYCARGGSILWWLIDYW |
| | | | | | | | | | GQGTLVTVSS |
| COV2- | Ab | SARS-CoV2 | SARS-CoV1 | | SARS-CoV1, | S; NTD | B-cells; SARS-CoV2 | 595 | EVQLLESGGGLVQPGGSLRLSCAASGFT |
| 2830 | | | | | SARS-CoV2 | | Human Patient | | FTSYAMSWVRQAPGKGLEWVSGISISG |
| | | | | | | | | | GSTYYAASVKGRFTISRDNSKNTLYLQM |
| | | | | | | | | | NSLRAEDTAVYYCAKDSRSGIAGVDAFD |
| | | | | | | | | | IWGQGTMVTVSS |
| COV2- | Ab | SARS-CoV2 | SARS-CoV1 | SARS-CoV2 | | S; RBD | B-cells; SARS-CoV2 | 596 | EVQLVESGGGLVQPGGSLRLSCAASGLT |
| 2832 | | | | | | | Human Patient | | VSSNYMSWVRQAPGKGLECVSVIYAGG |
| | | | | | | | | | NTYYADSVKGRFTISRDNSKNTLYLQMN |
| | | | | | | | | | SLRAEDTAVYYCARGDGGYYSPFDYWG |
| | | | | | | | | | QGTLVTVSS |
| COV2- | Ab | SARS-CoV1, | | | SARS-CoV2 | S; RBD | B-cells; SARS-CoV2 | 597 | QITLKESGPTLVKPTQTLTLTCTFSGFSL |
| 2834 | | SARS-CoV2 | | | | | Human Patient | | STSGVGVAWIRQPPGKALEWLALIYWDD |
| | | | | | | | | | DKRYSPSLKSRLTITKDTSKNQVVLTMTN |
| | | | | | | | | | MDPVDTATYYCAHRLPTPQLLPSFENW |
| | | | | | | | | | FDPWGQGTLVTVSS |
| COV2- | Ab | SARS-CoV2 | SARS-CoV1 | SARS-CoV2 | | S; RBD | B-cells; SARS-CoV2 | 598 | EVQLVESGGGLVQPGGSLRLSCAASGLT |
| 2835 | | | | | | | Human Patient | | VGSNYMNWVRQAPGKGLEWVSVIYSG |
| | | | | | | | | | GSTFYADSVKGRFTISRDNSKNTLYLQLN |
| | | | | | | | | | SLRAEDTAVYYCAREVVGYFDCWGQGT |
| | | | | | | | | | LVTVSS |
| COV2- | Ab | SARS-CoV2 | SARS-CoV1 | SARS-CoV2 | | S; RBD | B-cells; SARS-CoV2 | 599 | QVQLQESGPGLVKPSETLSLTCTVSGGSI |
| 2841 | | | | | | | Human Patient | | SSYYWSWIRQPPGKGLEWIGHIYYTGSS |
| | | | | | | | | | YYNPSLKSRVTISLDTSKNQFSLKLNSVT |
| | | | | | | | | | AADTAVYYCARLRWLRGGIDFWGQGTLV |
| | | | | | | | | | IVSS |
| COV2- | Ab | SARS-CoV1, | | | SARS-CoV2 | S; RBD | B-cells; SARS-CoV2 | 600 | QVQLVQSGAEVKKPGASVKVSCKASGD |
| 2842 | | SARS-CoV2 | | | | | Human Patient | | TFTTYYIHWVRQAPGQGLEWMGIINPS |
| | | | | | | | | | GGSRSYAQKFQGRISMTSDTSTSTVYME |
| | | | | | | | | | LSSLRSEDTAVYYCARGYGFVPNVLYYFD |
| | | | | | | | | | YWGQGILVTVSS |
| COV2- | Ab | SARS-CoV1, | | | SARS-CoV2 | S; non- | B-cells; SARS-CoV2 | 601 | QVELVESGGGVVQPGRSLRLSCAASGFI |
| 2844 | | SARS-CoV2 | | | | RBD | Human Patient | | FSSYAMHWVRQAPGKGLEWVAVISYD |
| | | | | | | | | | GGNKYYADSVKGRFTISRDNSKNTLYLQ |
| | | | | | | | | | MNSLRAEDTAVYYCARAQGGNYYYGM |
| | | | | | | | | | DVWGQGTTVTVSS |
| COV2- | Ab | SARS-CoV1, | | SARS-CoV2 | | S; RBD | B-cells; SARS-CoV2 | 602 | QVQLVESGGGVVQPGRSLRLSCAASGF |
| 2848 | | SARS-CoV2 | | (weak) | | | Human Patient | | TFSRYAMYWVRQAPGKGLEWVALISYD |
| | | | | | | | | | GRNEYYADSVKGRFTISRDNSKNTLYLQ |
| | | | | | | | | | MNSLRAGDSAVYYCARDLAYHPYRDYG |
| | | | | | | | | | DDDYYYYYGMDVWGQGTTVTVSS |
| COV2- | Ab | SARS-CoV2 | SARS-CoV1 | | SARS-CoV1, | S; RBD | B-cells; SARS-CoV2 | 603 | QVQLVQSGAEVKKPGASVKVSCKASGY |
| 2853 | | | | | SARS-CoV2 | | Human Patient | | TFTSYYMHWVRQAPGQGLEWMGIINP |
| | | | | | | | | | GGGSTTYAQKFQGRVTMTSDTSTSTVY |
| | | | | | | | | | MELSSLRSEDTAMYYCARGAIPPNSRAE |
| | | | | | | | | | IDYWGQGTLVTVSS |
| COV2- | Ab | SARS-CoV1, | | | SARS-CoV2 | S; RBD | B-cells; SARS-CoV2 | 604 | QVQLVQSGAEVKKPGASVKVSCKASGY |
| 2863 | | SARS-CoV2 | | | | | Human Patient | | TFTSYYLHWVRQAPGQGLEWMGIINPS |
| | | | | | | | | | GGSTTYAQKFQGRVTMTRDTSTSAVYM |
| | | | | | | | | | ELRSLRSEDTAVYYCARENDYGDYVEPR |
| | | | | | | | | | DYYYGMDVWGQGTTVTVSS |
| COV2- | Ab | SARS-CoV2 | SARS-CoV1 | | SARS-CoV1, | S; non- | B-cells; SARS-CoV2 | 605 | QVQLVESGGGVVQPGRSLRLSCAASGF |
| 2872 | | | | | SARS-CoV2 | RBD | Human Patient | | TFSTYGMHWVRQAPGKGLEWVAVIWY |
| | | | | | | | | | DGNKKYCADSVKGRCTISRDNSKNTLYL |
| | | | | | | | | | QMNSLRAEDTAVYYCAREGPFGDREAS |
| | | | | | | | | | GAFDVWGQGTMVTVSS |
| COV2- | Ab | SARS-CoV1, | | | SARS-CoV2 | S; RBD | B-cells; SARS-CoV2 | 606 | QVQLVESGGGLVKPGGSLRLSCAASGFT |
| 2873 | | SARS-CoV2 | | | | | Human Patient | | FSDYYMSWIRQAPGKGLEWVSYISSSGI |
| | | | | | | | | | TIYYADSVKGRFTISRDNAKNSLYLQMNS |
| | | | | | | | | | LRAEDTAVYYCTGVVAAPAEYFQHWGQ |
| | | | | | | | | | GTLVTVSS |
| COV2- | Ab | SARS-CoV1, | | SARS-CoV2 | | S; RBD | B-cells; SARS-CoV2 | 607 | EVQLVESGGGLVQPGGSLRLSCAASGFT |
| 2878 | | SARS-CoV2 | | (weak) | | | Human Patient | | FSRHWMTWVRQAPGKGLEWVANIKQ |
| | | | | | | | | | DGSEKYYVDSVKGRLTISRDNAKNSLYL |
| | | | | | | | | | QMNSLRAEDTAVYYCARLGFYYGGADY |
| | | | | | | | | | WGQGTLVTVSS |
| COV2- | Ab | SARS-CoV1, | | | SARS-CoV2 | S; non- | B-cells; SARS-CoV2 | 608 | QVQLVESGGGVVQPGRSLRLSCAASGF |
| 2883 | | SARS-CoV2 | | | | RBD | Human Patient | | TFSTYGMHWVRQAPGKGLEWVAVISY |
| | | | | | | | | | DGSNKYYADSVKGRFTISRDNSKNTLYL |
| | | | | | | | | | QMNSLRAEDTAMYYCAKDGSIAAADY |
| | | | | | | | | | WGQGTLVTVSS |
| COV2- | Ab | SARS-CoV2 | SARS-CoV1 | | SARS-CoV1, | S; RBD | B-cells; SARS-CoV2 | 609 | QVQLVQAGAEVKKPGASVKVSCKASGY |
| 2891 | | | | | SARS-CoV2 | | Human Patient | | TFTSYYMHWVRQAPGQGLEWMGIINP |
| | | | | | | | | | SGGSTSYAQKFQGRVTMTRDTSTSTVY |
| | | | | | | | | | MELSSLRSEDTAVYYCARGAAVPAAGEF |
| | | | | | | | | | DYWGQGTLVTVSS |
| COV2- | Ab | SARS-CoV1, | | | SARS-CoV2 | S; RBD | B-cells; SARS-CoV2 | 610 | QVQLVESGGGVVQSGGSLRLSCAASGF |
| 2894 | | SARS-CoV2 | | | | | Human Patient | | TFSTYGMHWVRQAPGKGLEWVAIISYD |
| | | | | | | | | | GINKYYADSVKGRFTISRDNSKNTVYLQ |
| | | | | | | | | | MNSLRTEDTAMYYCAKGDGSYLMDYF |
| | | | | | | | | | DYWGQGTLVTVSS |
| COV2- | Ab | SARS-CoV2 | SARS-CoV1 | | SARS-CoV1, | S; RBD | B-cells; SARS-CoV2 | 611 | EVQLVESGGGLVQPGGSLRLSCAASGFT |
| 2901 | | | | | SARS-CoV2 | | Human Patient | | VSSNYMSWVRQAPGKGLEWVSVIYPG |
| | | | | | | | | | GSAFYADSVKGRFTISRHNSNNTLCLQM |
| | | | | | | | | | NSLRTEDTAVYYCARSYDILTGYRDAFDI |
| | | | | | | | | | WGQGTMVTVSS |
| COV2- | Ab | SARS-CoV1, | | | SARS-CoV2 | S; non- | B-cells; SARS-CoV2 | 612 | QVQLQQWGAGLLKPSETLSLTCAVYGG |
| 2904 | | SARS-CoV2 | | | | RBD | Human Patient | | SFSGYYWSWIRQPPGKGLEWIGEINHS |
| | | | | | | | | | GSTNYNPSLKSRVTISVDTSKNHFSLKM |
| | | | | | | | | | NSVTAADTAVYYCARCRQMGNFYYYYM |
| | | | | | | | | | DVWGKGTTVTVSP |
| COV2- | Ab | SARS-CoV1, | | | SARS-CoV2 | S; RBD | B-cells; SARS-CoV2 | 613 | EVQLVQSGAEVKKPGESLKISCKGSGYSF |
| 2906 | | SARS-CoV2 | | | | | Human Patient | | TSYWVGWVRQMPGKGLEWMGIIYPG |
| | | | | | | | | | DSDTRDSPSFQGQVTISADKSISTAYLQ |
| | | | | | | | | | WSSLKASDTAMYYCARLGSESKIDYYYY |
| | | | | | | | | | GMDVWGQGTTVTVSS |
| COV2- | Ab | SARS-CoV2 | SARS-CoV1 | | SARS-CoV1, | S; RBD | B-cells; SARS-CoV2 | 614 | QVQLVQSGAEVKKPGSSVKVSCKASGD |
| 2909 | | | | | SARS-CoV2 | | Human Patient | | TFSSYTINWVRQAPGQGLEWMGRIIPIL |
| | | | | | | | | | GIPNYAQKFQGRVTITADKSTSTAFMEL |
| | | | | | | | | | SSLRSEDTAVYYCARGRGYSNYGASYYM |
| | | | | | | | | | DVWGKGTTVTVSS |
| COV2- | Ab | SARS-CoV2 | SARS-CoV1 | | SARS-CoV1, | S; RBD | B-cells; SARS-CoV2 | 615 | QLQLRESGPGLVKPSETLSLTCTVSGGSI |
| 2911 | | | | | SARS-CoV2 | | Human Patient | | SSSSYYWGWIRQPPGKGLEWIGSIYYSG |
| | | | | | | | | | STYYNPSLKSRVTISVDTSKNQFSLKLSS |
| | | | | | | | | | VTAADTAVFYCARMSRGYNYAYTFDIWG |
| | | | | | | | | | QGTMVTVSS |
| COV2- | Ab | SARS-CoV2 | SARS-CoV1 | SARS-CoV2 | | S; RBD | B-cells; SARS-CoV2 | 616 | QVTLRESGPALVKPTQTLTLTCTFSGFSL |
| 2919 | | | | | | | Human Patient | | STSGMCLSWIRQPPGKALEWLARIDWD |
| | | | | | | | | | DDKYYSTSLETRLTISKDTSKNQVVLTMT |
| | | | | | | | | | NMDPVDTGTYYCARTMATINAFDIWG |
| | | | | | | | | | QGTMVTVSS |
| COV2- | Ab | SARS-CoV1, | | | SARS-CoV2 | S; non- | B-cells; SARS-CoV2 | 617 | QVQLVESGGGVVQPGRSLRLSCAASGF |
| 2933 | | SARS-CoV2 | | | | RBD | Human Patient | | TFSYYPMHWLWVRQAPGKGLEWVAVT |
| | | | | | | | | | SYDGTNKYYADSVKGRFTISRDNSKNTLY |
| | | | | | | | | | LQMNSLRAEDTAVYYCARGGATNFDY |
| | | | | | | | | | WGQGTLVTVSS |
| COV2- | Ab | SARS-CoV2 | SARS-CoV1 | | SARS-CoV1, | S; non- | B-cells; SARS-CoV2 | 618 | QVQLVQSGSELKKPGASVKVSCKASEYT |
| 2934 | | | | | SARS-CoV2 | RBD | Human Patient | | FTSYAMNWVRQAPGQGLEWMGWINT |
| | | | | | | | | | NTGNPTYAQGFTGRFVFSLDTSVSTAYL |
| | | | | | | | | | QISSLKAEDTAVYYCARPGKAAAFDYWG |
| | | | | | | | | | QGTLVTVSS |
| COV2- | Ab | SARS-CoV2 | SARS-CoV1 | | SARS-CoV1, | S; RBD | B-cells; SARS-CoV2 | 619 | QVQLVESGGGVVQPGRSLRLSCAASGF |
| 2939 | | | | | SARS-CoV2 | | Human Patient | | TFSSYGMHWVRQAPGKGLEWVAVIWY |
| | | | | | | | | | DGSNKYYAESVKGRFTISRDNSKNTLYLQ |
| | | | | | | | | | MNSLRAEDTAVYYCARDLHQDWVVVV |
| | | | | | | | | | AANVYGMDVWGQGTTVTVSS |
| COV2- | Ab | SARS-CoV2 | SARS-CoV1 | SARS-CoV2 | | S; RBD | B-cells; SARS-CoV2 | 620 | QMQLVQSGPEVKKPGTSVKVSCKASGF |
| 2941 | | | | (weak) | | | Human Patient | | TFMSSAVQWVRQARGQRLEWIGWIVI |
| | | | | | | | | | GSGNTNYAQKFQERVTITRDMSTSTAY |
| | | | | | | | | | MELSSLRSEDTAVYYCAAPYCSSISCNDG |
| | | | | | | | | | FDIWGQGTMVTVSS |
| COV2- | Ab | SARS-CoV2 | SARS-CoV1 | | SARS-CoV1, | S; non- | B-cells; SARS-CoV2 | 621 | QVTLRESGPALVKPTQTLTLTCTFSGFSL |
| 2942 | | | | | SARS-CoV2 | RBD | Human Patient | | STSGLCVSWIRQPPGKALEWLARIDWDD |
| | | | | | | | | | DKYYNTSLRTRLTISKDTSKNQVVLTMTN |
| | | | | | | | | | MDPVDTATYYCARATTFFYGMDVWGQ |
| | | | | | | | | | GTTATVSS |
| COV2- | Ab | SARS-CoV1, | | | SARS-CoV2 | S; RBD | B-cells; SARS-CoV2 | 622 | QVQLVESGGGVVQPGRSLRLSCAASGF |
| 2944 | | SARS-CoV2 | | | | | Human Patient | | TFRRYGMYWVRQAPGKGLEWVAVISY |
| | | | | | | | | | DGTDKYYTDSVKGRFTISRDNSKNTLYLQ |
| | | | | | | | | | MNSLRAEDTAVYYCAKKGGPYCGGGN |
| | | | | | | | | | CYAGYFDYWGQGILVTVSS |
| COV2- | Ab | SARS-CoV1, | | | SARS-CoV2 | S; RBD | B-cells; SARS-CoV2 | 623 | EVQVVESGGGLVQPGRSLRLSCAASGFT |
| 2945 | | SARS-CoV2 | | | | | Human Patient | | FEDYAMHWVRQAPGKGLEWVSGVSW |
| | | | | | | | | | NSGIIGYADSVKGRFTISRDNAKNSLYLQ |
| | | | | | | | | | MNSLRAEDTALYYCAKLDVGGYDFVSG |
| | | | | | | | | | HYYAFDIWGQGTMVTVSS |
| COV2- | Ab | SARS-CoV2 | SARS-CoV1 | | SARS-CoV1, | S; non- | B-cells; SARS-CoV2 | 624 | QVQLVQSGSELKKPGASVKVSCKASGYT |
| 2947 | | | | | SARS-CoV2 | RBD | Human Patient | | FSDYAMNWVRQAPGQGLEWMGWIN |
| | | | | | | | | | TNTGNPTYAQGFTGRFVFSLDTSVSTAY |
| | | | | | | | | | LQISSLKAEDTAVYYCARGLISLFRGAIF |
| | | | | | | | | | HYYYGMDVWGQGTTVTVSS |
| COV2- | Ab | SARS-CoV2 | SARS-CoV1 | SARS-CoV2 | | S; RBD | B-cells; SARS-CoV2 | 625 | EVQLVESGGGLVQPGGSLRLSCAASGLT |
| 2952 | | | | | | | Human Patient | | VRSNYMTWVRQTPGKGLEWVSVIYSG |
| | | | | | | | | | GSTFYADSVKGRFTISRDNSKNTVYLQM |
| | | | | | | | | | NSLRAEDTAVYYCARDLVTYGLDVWGQ |
| | | | | | | | | | GTTVTVSS |
| COV2- | Ab | SARS-CoV1, | | | SARS-CoV2 | S; RBD | B-cells; SARS-CoV2 | 626 | QITLKESGPTLVKPTQTLTLTCTFSGFSL |
| 2953 | | SARS-CoV2 | | | | | Human Patient | | STKRVGVGWIRQPPGKALEWLALIYWDD |
| | | | | | | | | | DQRYSPSLKSRLTITKDTSKNQVVLTMT |
| | | | | | | | | | NMDPVDTATYYCAHSGPPDLSPVLSQG |
| | | | | | | | | | WFDPWGQGTLVTVSS |
| COV2- | Ab | SARS-CoV2 | SARS-CoV1 | SARS-CoV2 | | S; RBD | B-cells; SARS-CoV2 | 627 | QVQLVESGGGVVQPGRSLRLSCAASGF |
| 2955 | | | | | | | Human Patient | | TFVTSGIHWVRQAPGKGLEWVAVISYD |
| | | | | | | | | | GSNKYYADSVKGRFTISRDNSKNTLYLQ |
| | | | | | | | | | MNSLRAEDTAVYYCAKGGPNKEVLYFG |
| | | | | | | | | | ELLDYGMDVWGQGTTVTVSS |
| COV2- | Ab | SARS-CoV1, | | | SARS-CoV2 | S; non- | B-cells; SARS-CoV2 | 628 | QVQVVQSGAEVKKPGASVKVSCKASGY |
| 2960 | | SARS-CoV2 | | | | RBD | Human Patient | | TFKNYGISWVRQAPGQGLEWMGWISA |
| | | | | | | | | | YTGNTNYAQKFQGRMTMTTDTSTGTG |
| | | | | | | | | | YMELRSLRSDDTAVYYCARVQRRRLDY |
| | | | | | | | | | WGQGTLVIVSS |
| COV2- | Ab | SARS-CoV2 | SARS-CoV1 | SARS-CoV2 | | S; RBD | B-cells; SARS-CoV2 | 629 | QMQLVQSGPEVKKPGTSVKVSCKASGF |
| 2961 | | | | | | | Human Patient | | TFMSSAVQWVRQARGQRLEWIGWIVI |
| | | | | | | | | | GSGNTNYAQKFQERVTITRDMSTSTAY |
| | | | | | | | | | MELSSLRSEDTAVYYCAAPYCSSISCNDG |
| | | | | | | | | | FDIWGQGTMVTVSS |
| COV2- | Ab | SARS-CoV2 | SARS-CoV1 | | SARS-CoV1, | S; RBD | B-cells; SARS-CoV2 | 630 | QVQLVQSGAEVKKPGASVKVSCKASGY |
| 2997 | | | | | SARS-CoV2 | | Human Patient | | TFTSYYMHWVRQAPGQGLEWMGIINP |
| | | | | | | | | | GGGSTTYAQKFQGRVTMTSDTSTSTVY |
| | | | | | | | | | MELSSLRSEDTAMYYCARGAIPPNSRAE |
| | | | | | | | | | IDYWGQGTLVTVSS |
| COV2- | Ab | SARS-CoV2 | SARS-CoV1 | | SARS-CoV1, | S; RBD | B-cells; SARS-CoV2 | 631 | QVQLVESGGGVVQPGRSLRLSCAASGF |
| 3010 | | | | | SARS-CoV2 | | Human Patient | | TFSDYAMHWVRQAPGKGLEWVADISF |
| | | | | | | | | | DGSNKYYADSVKGRFTISRDSSENTLYLQ |
| | | | | | | | | | MDSLRADDTAVYYCARDLSTTWYLEM |
| | | | | | | | | | WGPDAFDIWGQGTVVTVSS |
| COV2- | Ab | SARS-CoV2 | SARS-CoV1 | | SARS-CoV1, | S; RBD | B-cells; SARS-CoV2 | 632 | EVQLVESGGGLVQPGGSLRLSCAASGFT |
| 3012 | | | | | SARS-CoV2 | | Human Patient | | FSSYDLHWVRQGTGKRLEWVSAIGTAG |
| | | | | | | | | | DTYYLGSVKGRFTISRENAKNSLYLQMN |
| | | | | | | | | | SLRAGDTAVYYCARVLYDSSGFYNWFDP |
| | | | | | | | | | WGQGTLVTVSS |
| COV2- | Ab | SARS-CoV2 | SARS-CoV1 | | SARS-CoV1, | S; RBD | B-cells; SARS-CoV2 | 633 | QVQLVESGGGVVQPGRSLRLSCAASGF |
| 3013 | | | | | SARS-CoV2 | | Human Patient | | TFSNYGMHWVRQAPGKGLEWVAVMS |
| | | | | | | | | | YDGSNKYYADSVKGRFTISRDNSKNTLYL |
| | | | | | | | | | QMNSLRAEDTAVYYCAKNLGPYCSGGT |
| | | | | | | | | | CYSLVGDYWGQGTLVTVSS |
| COV2- | Ab | SARS-CoV2 | SARS-CoV1 | SARS-CoV2 | | S; RBD | B-cells; SARS-CoV2 | 634 | EVQLVQSGPEVKKPGTSVKVSCKASGFT |
| 3025 | | | | | | | Human Patient | | FMSSAVQWVRQARGQRLEWIGWIVIG |
| | | | | | | | | | SGNTNYAQKFQERVTITRDMSTSTAYM |
| | | | | | | | | | ELSSLRSEDTAVYYCAAPYCSSISCNDGF |
| | | | | | | | | | DIWGQGTMVTVSS |
| COV2- | Ab | SARS-CoV1, | | | SARS-CoV2 | S; RBD | B-cells; SARS-CoV2 | 635 | EVQLVESGGGVVQPGGSLRLSCAASGF |
| 3029 | | SARS-CoV2 | | | | | Human Patient | | TFDDYAMHWVRQAPGKGLEWVSLISG |
| | | | | | | | | | DGGNTYYADSVKGRFTISRDNSKNSLYL |
| | | | | | | | | | QMNSLRTEDTALYYCAKDEMAYPPSHH |
| | | | | | | | | | YYYYYMDVWGKGTTVTVSS |
| COV2- | Ab | SARS-CoV1, | | | SARS-CoV2 | S; non- | B-cells; SARS-CoV2 | 636 | EVQLVESGGGLVQPGGSLRLSCAASGFT |
| 3057 | | SARS-CoV2 | | | | RBD | Human Patient | | FSSYWMSWVRQAPGKGLEWVANIKQ |
| | | | | | | | | | DGSEKYFVDSVKGRFTISRDNAKNSLYL |
| | | | | | | | | | QMNSLRAEDAAVYYCARLVTTVTTANG |
| | | | | | | | | | LYYYSYYYMDVWGKGTTVTVSS |
| COV2- | Ab | SARS-CoV1, | | | SARS-CoV2 | S; non- | B-cells; SARS-CoV2 | 637 | EVQLVESGGGAVQPGRSLRLSCAASGFT |
| 3058 | | SARS-CoV2 | | | | RBD | Human Patient | | FSTYAMYWVRQAPGKGLEWVAVISYD |
| | | | | | | | | | GSNRYYADSVKGRFTISRDNSKNTLYLQ |
| | | | | | | | | | MNSLRPEDTAVYYCARDRSGNYRDAFD |
| | | | | | | | | | IWGQGTMVTVSS |
| COVA1- | Ab | SARS-CoV2 | SARS-CoV1 | | SARS-CoV1, | S; non- | B-cells; SARS-CoV2 | 638 | HQHQVEQGPGGVEQSETLFLTCCVSGG |
| 01 | | | | | SARS-CoV2 | RBD | Human Patient | | FTISSCYKRGGIRQPPRKGEEWAVSSYYS |
| | | | | | | | | | SSTYYTPSLKSRVTISVDKSKNQFSLKMS |
| | | | | | | | | | SVTAADTAVYYCARVSSGYYFTPFDYWG |
| | | | | | | | | | QGTRGHRLF |
| COVA1- | Ab | SARS-CoV2 | SARS-CoV1 | | SARS-CoV1, | S; non- | B-cells; SARS-CoV2 | 639 | EVQLVESGGGVVQPGRSLRLSCAASGFT |
| 02 | | | | | SARS-CoV2 | RBD | Human Patient | | FSIYAMHWVRQAPGKGLEWVAVISYD |
| | | | | | | | | | GSNQYYADSVRGRFTISRGNSKNTLYLQ |
| | | | | | | | | | MNSLRPEDTAVYYCARARGGSYNDAFD |
| | | | | | | | | | IWGQGTMVTVSS |
| COVA1- | Ab | SARS-CoV2 | SARS-CoV1 | SARS-CoV2 | SARS-CoV1 | link | B-cells; SARS-CoV2 | 640 | EVQLVESGGGVVQPGRSLRLSCAASGFT |
| 03 | | | | | | | Human Patient | | FSSYGMHWVRQAPGKGLEWVAVISYD |
| | | | | | | | | | GSNKYYADSVKGRFTISRDNSKNTLYLQ |
| | | | | | | | | | MNSLRAEDTAVYYCAKDGQYYDFWSGY |
| | | | | | | | | | LGARTNPHYYYYMDVWGKGTMVTVSS |
| COVA1- | Ab | SARS-CoV2 | SARS-CoV1 | | SARS-CoV1, | S; non- | B-cells; SARS-CoV2 | 641 | EVQLVQSGGGLVQPGRSLRLSCAASGFT |
| 06 | | | | | SARS-CoV2 | RBD | Human Patient | | FGEYAMHWVRQAPGKGLEWVSGISW |
| | | | | | | | | | NSGSIDYADSVKGRFTISRDNAKNSLYLQ |
| | | | | | | | | | MNSLRAEDTALYYCAKDMGEAVAGTHY |
| | | | | | | | | | GMDVWGQGTMVTASS |
| COVA1- | Ab | SARS-CoV1, | | | SARS-CoV1, | S; non- | B-cells; SARS-CoV2 | 642 | QVQLVESGAEVKKPGSSVKVSCKASGGT |
| 07 | | SARS-CoV2 | | | SARS-CoV2 | RBD | Human Patient | | LSSYAITWVRQAPGQGLEWVGGIIPIFG |
| | | | | | | | | | TANYAQKFQGRVTITADESTSTAYMELS |
| | | | | | | | | | SLRSEDTAVYYCARVGAYDSSGYSNDYW |
| | | | | | | | | | GQGTLVTVSS |
| COVA1- | Ab | SARS-CoV2 | SARS-CoV1 | | SARS-CoV1, | S; RBD | B-cells; SARS-CoV2 | 643 | EVQLVESGGGVVQPGGSLRLSCAASGF |
| 08 | | | | | SARS-CoV2 | | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCAREDYYDSSGSF |
| | | | | | | | | | DYWGQGTLVTVSS |
| COVA1- | Ab | SARS-CoV2 | SARS-CoV1 | | SARS-CoV1, | S; non- | B-cells; SARS-CoV2 | 644 | QLQLVESGPGLVKPSETLSLTCTVSGGSI |
| 09 | | | | | SARS-CoV2 | RBD | Human Patient | | SSYFWSWIRQPPGKGLEWIGYIYYSGST |
| | | | | | | | | | NYNPSLKSRVTISVDTSKNQFSLKLSSVT |
| | | | | | | | | | AADTAVYYCARHSQGWLQQAVAFDIWG |
| | | | | | | | | | QGTMVTVSS |
| COVA1- | Ab | SARS-CoV2 | SARS-CoV1 | | SARS-CoV1, | S; RBD | B-cells; SARS-CoV2 | 645 | EVQLVESGGGLVQPGGSLRLSCAASGFT |
| 10 | | | | | SARS-CoV2 | | Human Patient | | VTSTYMTWVRRAPGKGLEWVSIIYNDD |
| | | | | | | | | | TTYYADSVKDRVTVSRDDSKNTLYLQM |
| | | | | | | | | | NSLRAEDTAIYYCARGGYYYDPSGYYSRS |
| | | | | | | | | | FSFDYWGQGTLVTVSS |
| COVA1- | Ab | SARS-CoV2 | SARS-CoV1 | SARS-CoV2 | SARS-CoV1 | S; RBD | B-cells; SARS-CoV2 | 646 | EVQLVQSGTEVKKPGASVKVSCKASGYT |
| 12 | | | | | | | Human Patient | | FPGYYIHWVRQAPGQGLEWMGWINP |
| | | | | | | | | | NSGVAKSAQKFQGRVTMTRDSSISTVYL |
| | | | | | | | | | DVTSDDTAVYYCARDLVWATVSGTMD |
| | | | | | | | | | VWGQGTTVTVSS |
| COVA1- | Ab | SARS-CoV1, | | SARS-CoV1, | | S; RBD | B-cells; SARS-CoV2 | 647 | QVQLVQSGAEVKKPGASVKVSCKASGY |
| 16 | | SARS-CoV2 | | SARS-CoV2 | | | Human Patient | | TFTSYYMHWVRQAPGQGLEWMGIINS |
| | | | | | | | | | SGGSTSYAQKFQGRVTMTRDTSTSTVY |
| | | | | | | | | | MELSSLRSEDTAVYYCARPPRNYYDRSG |
| | | | | | | | | | YYQRAEYFQHWGQGTLVTVSS |
| COVA1- | Ab | SARS-CoV2 | SARS-CoV1 | SARS-CoV2 | SARS-CoV1 | S; RBD | B-cells; SARS-CoV2 | 648 | EVQLVESGGGLVQPGGSLRLSCAASGFT |
| 18 | | | | | | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAEDTAVYYCARVEWAAAGTFYWG |
| | | | | | | | | | QGTLVTVSS |
| COVA1- | Ab | SARS-CoV2 | SARS-CoV1 | | SARS-CoV1, | S; non- | B-cells; SARS-CoV2 | 649 | QVQLVESGGGLVKPGGSLRLSCAASGFT |
| 19 | | | | | SARS-CoV2 | RBD | Human Patient | | FSSYSMNWVRQAPGKGLEWVSSISSSSS |
| | | | | | | | | | FIYYADSVKGRFTISRDNAKNSLYLQMNS |
| | | | | | | | | | LRAEDTAVYYCARWKSDYYDSSGYYPAA |
| | | | | | | | | | FDIWGQRDKWSPSS |
| COVA1- | Ab | SARS-CoV2 | SARS-CoV1 | | SARS-CoV1, | S; non- | B-cells; SARS-CoV2 | 650 | QVQLVESGGGLVKPGGSLRLSCAASGFT |
| 20 | | | | | SARS-CoV2 | RBD | Human Patient | | FSSYSMNWVRQAPGKGLEWVSSISSSSS |
| | | | | | | | | | YIYYADSVKGRFTISRDDAKNSLFLQMNS |
| | | | | | | | | | LRAEDTAVYYCAGDQNLYCSGDSCYYHY |
| | | | | | | | | | YGMDVWGQGTVVTVSS |
| COVA1- | Ab | SARS-CoV2 | SARS-CoV1 | SARS-CoV2 | SARS-CoV1 | S; non- | B-cells; SARS-CoV2 | 651 | EVQLVESGGGVVQPGRSLRLSCAASGFT |
| 21 | | | | | | RBD | Human Patient | | FSSYAMHWVRQAPGKGLEWVAVISYD |
| | | | | | | | | | GSNKYYADSVKGRFTISRDNTKNTLYLQ |
| | | | | | | | | | MNSLRAEDTAVYYCARDSEYYDILTGYL |
| | | | | | | | | | APTHYYYYYMDVWGKGTTVTVSS |
| COVA1- | Ab | SARS-CoV2 | SARS-CoV1 | | SARS-CoV1, | S; non- | B-cells; SARS-CoV2 | 652 | EVQLVESGAEVKKPGASVKVSCKASGYT |
| 22 | | | | | SARS-CoV2 | RBD | Human Patient | | FTSYGISWVRQAPGQGLEWMGWISAY |
| | | | | | | | | | NGYTNSAQKLQGRVTMTTDTSTSTAYM |
| | | | | | | | | | ELRSLRSDDTAVYYCARDLVDTAMVQTL |
| | | | | | | | | | DDYGMDVWGQGTMVTVSS |
| COVA1- | Ab | SARS-CoV2 | SARS-CoV1 | | SARS-CoV1, | S; non- | B-cells; SARS-CoV2 | 653 | QVQLVQSGAEVKKPGESLKISCKGSGYR |
| 23 | | | | | SARS-CoV2 | RBD | Human Patient | | FTTYWIGWVRQMPGKGLEWMGIIYPG |
| | | | | | | | | | DSDTRYSPSFQGQVTISADKSISTAYLQW |
| | | | | | | | | | SSLTASDTAIYYCARYYYDSRGYTSIDFW |
| | | | | | | | | | GQGTLVTVSS |
| COVA1- | Ab | SARS-CoV2 | SARS-CoV1 | | SARS-CoV1, | S; non- | B-cells; SARS-CoV2 | 654 | EVQLVESGPGLVKPSETLSLTCTVSGGSI |
| 25 | | | | | SARS-CoV2 | RBD | Human Patient | | SSTSYYWGWIRQPPGKGLECIGSIYYSGS |
| | | | | | | | | | TYYNPSLKSRVTISVDTSKNQFSLKLSSV |
| | | | | | | | | | TAADTAVYYCARLNYDFWSGYYSYALYY |
| | | | | | | | | | MDVWGKGTMVTVSS |
| COVA1- | Ab | SARS-CoV2 | SARS-CoV1 | | SARS-CoV1, | S; non- | B-cells; SARS-CoV2 | 655 | QLQLQESGPGLVKPSQTLSLTCTVSGGSI |
| 26 | | | | | SARS-CoV2 | RBD | Human Patient | | SSGGYYWSWIRQLPGKGLEWIGYIYYSG |
| | | | | | | | | | STYYNPSLKSRVTISVDTSKNQFSLNLSS |
| | | | | | | | | | VTAADTAVYYCARQQLDYYDSSGCFDYW |
| | | | | | | | | | GQGTLVTVSS |
| COVA1- | Ab | SARS-CoV1, | | | SARS-CoV1, | S; non- | B-cells; SARS-CoV2 | 656 | EVQLVESGPGLVKPSETLSLTCTVSGGSI |
| 27 | | SARS-CoV2 | | | SARS-CoV2 | RBD | Human Patient | | SSYYWSWIRQPPGKRLEWIGYIYYSGST |
| | | | | | | | | | NYNPSLKSRVTISVDTSKNQFSLKLNSVT |
| | | | | | | | | | AADTAAYYCARGFDYWGQGTRVTVSS |
| COVA2- | Ab | SARS-CoV2 | SARS-CoV1 | | SARS-CoV1, | S; RBD | B-cells; SARS-CoV2 | 657 | EVQLVESGGGLVQPGGSLRLSCAASGFT |
| 01 | | | | | SARS-CoV2 | | Human Patient | | FSSYDMHWVRQTTGKGLQWVSAIGTA |
| | | | | | | | | | GDTYYPGSVKGRFTISRENAKNSLYLQM |
| | | | | | | | | | NSLRAGDTAVYYCARGGDRYPVGYFDL |
| | | | | | | | | | WGRGTLVTVSS |
| COVA2- | Ab | SARS-CoV1, | | SARS-CoV1, | | S; RBD | B-cells; SARS-CoV2 | 658 | VQLQQESGPGLVKPSETLSLTCTVSGGSI |
| 02 | | SARS-CoV2 | | SARS-CoV2 | | | Human Patient | | SSSSYYWGWIRQPPGKGLEWIGSIYYSG |
| | | | | (weak) | | | | | STYYNPSLKSRVTISVDTSKNQFSLKLSS |
| | | | | | | | | | VTAADTAVYYCARRSTSRWGYYYMDVW |
| | | | | | | | | | GKGTRVTVSS |
| COVA2- | Ab | SARS-CoV2 | SARS-CoV1 | | SARS-CoV1, | S; non- | B-cells; SARS-CoV2 | 659 | EVQLVESGGGLVQPGGSLRLSCAASGFT |
| 03 | | | | | SARS-CoV2 | RBD | Human Patient | | FSSYSMNWVRQAPGKGLEWVSYISSSSS |
| | | | | | | | | | TIYYADSVKGRFTISRDNAKNSLYLQMNS |
| | | | | | | | | | LRAEDTAVYYCAREANSDFWSGYLGYFD |
| | | | | | | | | | YWGQGTLVTVSS |
| COVA2- | Ab | SARS-CoV2 | SARS-CoV1 | SARS-CoV2 | SARS-CoV1 | S; RBD | B-cells; SARS-CoV2 | 660 | QVQLVETGGGLIQPGGSLRLSCAASGFT |
| 04 | | | | | | | Human Patient | | VSSNYMSWVRQAPGKGLEWVSVIYSG |
| | | | | | | | | | GSTFYADSVKGRFTISRDNSKNTLYLQM |
| | | | | | | | | | NSLRAEDTAVYYCARDLERAGGMDVW |
| | | | | | | | | | GQGTMVTVSS |
| COVA2- | Ab | SARS-CoV2 | SARS-CoV1 | | SARS-CoV1, | S; RBD | B-cells; SARS-CoV2 | 661 | EVQLLESGAEVKKPGESLKISCKGSGYSF |
| 05 | | | | | SARS-CoV2 | | Human Patient | | TSYWIGWVRQMPGKGLEWMGIIYPGD |
| | | | | | | | | | SDTRYSPSFQGQVTISADKSISTAYLQWS |
| | | | | | | | | | SLKASDTAMYYCARHMRPSIAARPGYQ |
| | | | | | | | | | YYMDVWGKGTRVTVSS |
| COVA2- | Ab | SARS-CoV2 | SARS-CoV1 | SARS-CoV2 | SARS-CoV1 | S; RBD | B-cells; SARS-CoV2 | 662 | EVQLVETGGGLIQPGGSLRLSCAASGLT |
| 07 | | | | | | | Human Patient | | VSSNYMNWVRQAPGKGLEWVSVIYSG |
| | | | | | | | | | GSTYYADSVKGRFTISRDNSKNTLYLQM |
| | | | | | | | | | NSLRAEDTAVYYCAREAYGMDVWGQG |
| | | | | | | | | | TMVTVSS |
| COVA2- | Ab | SARS-CoV2 | SARS-CoV1 | | SARS-CoV1, | S; non- | B-cells; SARS-CoV2 | 663 | EVQLLESGGGLVQPGGSLKLSCAASGFT |
| 10 | | | | | SARS-CoV2 | RBD | Human Patient | | FSSYAMSWVRQAPGKGLEWVSAISGSG |
| | | | | | | | | | SNTYYADSVKGRFTISRDNSKNTLYLQM |
| | | | | | | | | | NSLRAEDTAVYYCAKGLRGQQLVIPTEY |
| | | | | | | | | | FQHWGQGTLVTVSS |
| COVA2- | Ab | SARS-CoV2 | SARS-CoV1 | SARS-CoV2 | SARS-CoV1 | S; RBD | B-cells; SARS-CoV2 | 664 | EVQLVESGAEVKKPGSSVKVSCKASGGT |
| 11 | | | | (weak) | | | Human Patient | | LSSYAISWVRQAPGQGLEWMGGIIPIFG |
| | | | | | | | | | TANYAQKFQGRVTITTDESTSTAYMELSS |
| | | | | | | | | | LRSEDTAVYYCARGPRGCSSTSCYGSYFD |
| | | | | | | | | | YWGQGTLVTVSS |
| COVA2- | Ab | SARS-CoV2 | SARS-CoV1 | | SARS-CoV1, | S; non- | B-cells; SARS-CoV2 | 665 | EVQLVESGGGLVKPGGSLRLSCAASGFT |
| 12 | | | | | SARS-CoV2 | RBD | Human Patient | | FGSYSMSWVRQAPGKGLEWVSSISRSS |
| | | | | | | | | | SYIYNADSVRGRLTISRDNAKNSLYLQM |
| | | | | | | | | | NSLRVEDTAVYYCARDQPLPDILTGYYT |
| | | | | | | | | | GPLDYWGQGTLVTVSS |
| COVA2- | Ab | SARS-CoV2 | SARS-CoV1 | SARS-CoV2 | SARS-CoV1 | S; RBD | B-cells; SARS-CoV2 | 666 | QVQLVETGGGLIQPGGSLRLSCAASGFT |
| 13 | | | | (weak) | | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAEDTAVYYCARDLDTMGGMDVW |
| | | | | | | | | | GQGTRVTVSS |
| COVA2- | Ab | SARS-CoV1, | | | SARS-CoV1, | S; non- | B-cells; SARS-CoV2 | 667 | QVQLVQSGAEVKKPGSSVKVSCKASGG |
| 14 | | SARS-CoV2 | | | SARS-CoV2 | RBD | Human Patient | | TFSSYAIIWVRQAPGQGLEWMGGIIPIF |
| | | | | | | | | | GTANYAQKFQGRVTITTDESTSTAYMEL |
| | | | | | | | | | SSLRSEDTAVYYCARVRYYDSSGYYEDY |
| | | | | | | | | | WGQGTLVTVSS |
| COVA2- | Ab | SARS-CoV2 | SARS-CoV1 | SARS-CoV2 | SARS-CoV1 | S; RBD | B-cells; SARS-CoV2 | 668 | QVQLLESGGGLVQPGGSLRLSCAASGFT |
| 15 | | | | | | | Human Patient | | FSSYAMSWVRQAPGKGLEWASAISGSG |
| | | | | | | | | | GSTYYADSVEGRFTISRDNSKNTLYLQM |
| | | | | | | | | | NSLRAEDTAVYYCAKDTGYCGDDCYIKLI |
| | | | | | | | | | RGGPDYWGQGTLVTVSS |
| COVA2- | Ab | SARS-CoV2 | SARS-CoV1 | | SARS-CoV1, | S; RBD | B-cells; SARS-CoV2 | 669 | QVQLVQSGAEVKKPGATVKISCKVSGYT |
| 16 | | | | | SARS-CoV2 | | Human Patient | | FTDYYMHWVQQAPGKGLEWMGLVDP |
| | | | | | | | | | EDGETIYAEKFQGRVTITADTSTDTAYM |
| | | | | | | | | | ELSSLRSEDTAVYYCASSDSSGFVGSRGF |
| | | | | | | | | | DYWGQGTRVTVSS |
| COVA2- | Ab | SARS-CoV2 | SARS-CoV1 | | SARS-CoV1, | S; RBD | B-cells; SARS-CoV2 | 670 | QVQLVESGAEVKKPGSSVKVSCKASGGT |
| 17 | | | | | SARS-CoV2 | | Human Patient | | FSSYGINWVRQAPGQGLEWMGGIIPIF |
| | | | | | | | | | GTANYAQKFQGRVTITTDESTSTAYMEL |
| | | | | | | | | | SSLRSEDTAVYYCASFGDDSGDEGVRW |
| | | | | | | | | | GQGTLVTVSS |
| COVA2- | Ab | SARS-CoV1, | | SARS-CoV2 | SARS-CoV1 | S; non- | B-cells; SARS-CoV2 | 671 | EVQLVQSGAEVKKPGSSVKVSCKASGGT |
| 18 | | SARS-CoV2 | | | | RBD | Human Patient | | FSSYAISWVRQAPGQGLEWMGGIIPIFG |
| | | | | | | | | | TTNYAQKFQGRVTITTDESTSTAYMELSS |
| | | | | | | | | | LRSEDTAVYYCARVYSYDSSGYYLEYWG |
| | | | | | | | | | QGTRVTVSS |
| COVA2- | Ab | SARS-CoV2 | SARS-CoV1 | | SARS-CoV1, | S; RBD | B-cells; SARS-CoV2 | 672 | EVQLVESGGGLIQPGGSLRLSCAASGFT |
| 20 | | | | | SARS-CoV2 | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAEDTAVYYCASPLLLTPPDYYYYMD |
| | | | | | | | | | VWGKGTMVTVSS |
| COVA2- | Ab | SARS-CoV2 | SARS-CoV1 | | SARS-CoV1, | S; non- | B-cells; SARS-CoV2 | 673 | EVQLVESGPGLVKPSETLSLTCTVSGGSI |
| 22 | | | | | SARS-CoV2 | RBD | Human Patient | | SSYYWSWIRQPAGKGLEWIGRIYTSGST |
| | | | | | | | | | NYNPSLKSRVTMSVDTSKNQFSLKLSSV |
| | | | | | | | | | TAADTAVYYCARWKYNDRFDYWGQGT |
| | | | | | | | | | RVTVSS |
| COVA2- | Ab | SARS-CoV2 | SARS-CoV1 | | SARS-CoV1, | S; RBD | B-cells; SARS-CoV2 | 674 | EVQLVESGAEVKKPGSSVKVSCKASGGT |
| 23 | | | | | SARS-CoV2 | | Human Patient | | FSSYAISWVRQAPGQGLEWMGGIIPIFG |
| | | | | | | | | | TANYAQKFQGRVTITTDESTSTAYMELSS |
| | | | | | | | | | LRSEDTAVYYCARGPRYCSSTSCYAGVYF |
| | | | | | | | | | DYWGQGTRVTVSS |
| COVA2- | Ab | SARS-CoV2 | SARS-CoV1 | | SARS-CoV1, | S; RBD | B-cells; SARS-CoV2 | 675 | EVQLVQSGAEVKKPGESLRISCKGSGYSF |
| 24 | | | | | SARS-CoV2 | | Human Patient | | TSYWISWVRQMPGKGLEWMGRIDPSD |
| | | | | | | | | | SYTNYSPSFQGHVTISADKSISTAYLQWS |
| | | | | | | | | | SLKASDTAMYYCARPNPAGGYDSSGWV |
| | | | | | | | | | DAFDIWGQGTRVTVSS |
| COVA2- | Ab | SARS-CoV2 | SARS-CoV1 | | SARS-CoV1, | S; non- | B-cells; SARS-CoV2 | 676 | EVQLVESGAEVKKPGASVKVSCKVSGYT |
| 25 | | | | | SARS-CoV2 | RBD | Human Patient | | LPELSMHWVRQTPGKGLEWMGGFDP |
| | | | | | | | | | EDGETIYAQKFQGRVTMTEDTSTDTAY |
| | | | | | | | | | MELSSLRSEDTAVYYCATGPTIAAAATN |
| | | | | | | | | | WFDPGGQGTLVTVSS |
| COVA2- | Ab | SARS-CoV2 | SARS-CoV1 | | SARS-CoV1, | S; non- | B-cells; SARS-CoV2 | 677 | EVQLVESGGGLVKPGGSLRLSCAASGFT |
| 26 | | | | | SARS-CoV2 | RBD | Human Patient | | FSNAWMSWVRQAPGKGLEWVGRIKSK |
| | | | | | | | | | TDGGTTDYAAPVKGRFTISRDDSKNTLY |
| | | | | | | | | | LQMNSLKTEDTAVYYCTTDRGDSYGYYY |
| | | | | | | | | | CMDVWGKGTRVTVSS |
| COVA2- | Ab | SARS-CoV2 | SARS-CoV1 | | SARS-CoV1, | S; non- | B-cells; SARS-CoV2 | 678 | EVQLVESGGGVVQPGRSLRLSCAASGFT |
| 28 | | | | | SARS-CoV2 | RBD | Human Patient | | FSSYGMHWVRQAPGKGLEWVAVIWY |
| | | | | | | | | | DGSNKYYADSVKGRFTISRDNSKNTLYL |
| | | | | | | | | | QMDSLRAEDTAMYYCAKDKAPPCSSG |
| | | | | | | | | | WYYFDYWGQGTLVTVSS |
| COVA2- | Ab | SARS-CoV2 | SARS-CoV1 | SARS-CoV2 | SARS-CoV1 | S; RBD | B-cells; SARS-CoV2 | 679 | EVQLVESGGGVVQPGRSLRLSCAASGFT |
| 29 | | | | | | | Human Patient | | FSSYTMHWVRQAPGKGLEWVAVISYD |
| | | | | | | | | | GSNKYYADSVKGRFTISRDNSKNTLYLQ |
| | | | | | | | | | MNSLRAEDTAVYYCASLPVVPAAIGPLP |
| | | | | | | | | | AFDIWGQGTMVTVSS |
| COVA2- | Ab | SARS-CoV2 | SARS-CoV1 | | SARS-CoV1, | S; non- | B-cells; SARS-CoV2 | 680 | EVQLVESGPGLVKPSQTLSLTCTVSGGSI |
| 30 | | | | | SARS-CoV2 | RBD | Human Patient | | SSGDYYWSWIRQPPGKGLEWIGYIYYSG |
| | | | | | | | | | STYYNPSLKSRVTISVDTSKNQFSLKLSS |
| | | | | | | | | | VTAADTAVYYCARGVEDPVVPAAIPWCW |
| | | | | | | | | | FDPWGQGTRVTVSS |
| COVA2- | Ab | SARS-CoV2 | SARS-CoV1 | | SARS-CoV1, | S; RBD | B-cells; SARS-CoV2 | 681 | EVQLVESGVEVKKPGASVKVSCKAFGYT |
| 31 | | | | | SARS-CoV2 | | Human Patient | | FTGQDMHWVRQAPGQGLEWMGWIN |
| | | | | | | | | | PSSAGTNYAQKFQGRVTMTRDTSISTAY |
| | | | | | | | | | MELSRLRSDDTSVYYCRKMLTIFGKVNQ |
| | | | | | | | | | TMLLISGAKGQWSPSLQWGQGTMVTV |
| | | | | | | | | | SS |
| COVA2- | Ab | SARS-CoV1, | | | SARS-CoV1, | S; RBD | B-cells; SARS-CoV2 | 682 | EVQLVESGAEVKKPGSSVKVSCKASGGT |
| 32 | | SARS-CoV2 | | | SARS-CoV2 | | Human Patient | | FSSYAISWVRQAPGQGLEWMGGIIPIFG |
| | | | | | | | | | TANYAQKFQGRATITTDESTSTAYMELSS |
| | | | | | | | | | LGSEDTAVYYCARTHSYDNSGQYFDYW |
| | | | | | | | | | GQGTMVTVSS |
| COVA2- | Ab | SARS-CoV2 | SARS-CoV1 | | SARS-CoV1, | S; non- | B-cells; SARS-CoV2 | 683 | QVQLVQSGAEVKKPGESLRISCKGSGYS |
| 33 | | | | | SARS-CoV2 | RBD | Human Patient | | FTSHWISWVRQMPGKGLEWMGRIDPS |
| | | | | | | | | | DSYTNYSPSFQGHVTISADKSISTAYLQW |
| | | | | | | | | | SSLKASDTAMYYCARLKVITIFGVVRDDY |
| | | | | | | | | | GMDVWGQGTTVTVSS |
| COVA2- | Ab | SARS-CoV2 | SARS-CoV1 | | SARS-CoV1, | S; non- | B-cells; SARS-CoV2 | 684 | QVQLVESGGGVVQPGRSLRLSCAASGF |
| 34 | | | | | SARS-CoV2 | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARSASGSYYGAF |
| | | | | | | | | | DYWGQGTLVTVSS |
| COVA2- | Ab | SARS-CoV2 | SARS-CoV1 | | SARS-CoV1, | S; non- | B-cells; SARS-CoV2 | 685 | EVQLVESGAEVKKPGASVKVSCKVSGYT |
| 37 | | | | | SARS-CoV2 | RBD | Human Patient | | LPELSMHWVRQAPGKGLDWMGGFDP |
| | | | | | | | | | EDGETIYAQKFQGRVTMTEDTSTDTAY |
| | | | | | | | | | MELSSLRSEDTAVYYCATSPAVMSVGW |
| | | | | | | | | | VDPWGQGTLVTVSS |
| COVA2- | Ab | SARS-CoV2 | SARS-CoV1 | | SARS-CoV1, | S; non- | B-cells; SARS-CoV2 | 686 | QVQLQQSGPGLVKPSETLPLTCTVSGGS |
| 38 | | | | | SARS-CoV2 | RBD | Human Patient | | ISSSSYYWGWIRQPPGKGLEWIGSVFYS |
| | | | | | | | | | GSTYYNPSLKSRVTISVDTSKNQLSLKLS |
| | | | | | | | | | SVTAADTAVYYCARQVRQWLEDDAFDI |
| | | | | | | | | | WGQGTMVTVSS |
| COVA2- | Ab | SARS-CoV2 | SARS-CoV1 | | SARS-CoV1, | S; RBD | B-cells; SARS-CoV2 | 687 | QVQLVETGGGLIQPGGSLRLSCAASGFT |
| 39 | | | | | SARS-CoV2 | | Human Patient | | VSSNYMSWVRQAPGKGLEWVSVIYTG |
| | | | | | | | | | GTTYYADSVKGRFTISRDNSKNTLYLQM |
| | | | | | | | | | NSLRAEDTAVYYCARAHVDTAMVESGA |
| | | | | | | | | | FDIWGQGTRVTVSS |
| COVA2- | Ab | SARS-CoV2 | SARS-CoV1 | | SARS-CoV1, | S; non- | B-cells; SARS-CoV2 | 688 | QVQLQESGPGLVKPSGTLSLTCAVSGGS |
| 40 | | | | | SARS-CoV2 | RBD | Human Patient | | ISSSNWWSWVRQPPGKGLEWIGEIYHS |
| | | | | | | | | | GSSNYNPSLKSRVTISVDKSKNQFSLKLN |
| | | | | | | | | | SVTAADTAVYYCAGRYCSGGRCGWFDP |
| | | | | | | | | | WGQGTLVTVSS |
| COVA2- | Ab | SARS-CoV2 | SARS-CoV1 | | SARS-CoV1, | S; non- | B-cells; SARS-CoV2 | 689 | EVQLVESGGGLVKPGGSLRLSCSASGFT |
| 41 | | | | | SARS-CoV2 | RBD | Human Patient | | FSNYNMDWVRQAPGKGLEWVSSISSSS |
| | | | | | | | | | SYIYLADSVKGRFTISRDNAKNSLYLQMN |
| | | | | | | | | | SLRAEDTAVYYCARVQKDIVVVPVALAD |
| | | | | | | | | | YYYYGMDVWGQGTTVTVSS |
| COVA2- | Ab | SARS-CoV2 | SARS-CoV1 | | SARS-CoV1, | S; non- | B-cells; SARS-CoV2 | 690 | QVQLVQPGAEVKKPGASVKVSCKASGY |
| 43 | | | | | SARS-CoV2 | RBD | Human Patient | | TFTSYGISWVRQAPGQGLEWMGWISA |
| | | | | | | | | | YNGDTNYAQKLQGRVTMTTDTSTSTAY |
| | | | | | | | | | MELRSLKSDDTAVYYCARFDYGYPYSSW |
| | | | | | | | | | SVLSIDYWGQGTLVTVSS |
| COVA2- | Ab | SARS-CoV1, | | | SARS-CoV1, | S; RBD | B-cells; SARS-CoV2 | 691 | EVQLVESGGGVVQPGRSLRLSCAASGFT |
| 44 | | SARS-CoV2 | | | SARS-CoV2 | | Human Patient | | FSSYAMHWVRQAPGKGLEWVAVISYD |
| | | | | | | | | | GSYKYYADSVKGRFTISRDNSKNTLYLQ |
| | | | | | | | | | MNSLTAEDTAVYYCAREGSRQWLVIYF |
| | | | | | | | | | DYWGQGTLVTVSS |
| COVA2- | Ab | SARS-CoV2 | | | SARS-CoV1, | S; RBD | B-cells; SARS-CoV2 | 692 | EVQLLESGAEVKKPGASVKISCKASGYTF |
| 45 | | | | | SARS-CoV2 | | Human Patient | | TGYYMHWVRQAPGQGLEWMGWINP |
| | | | | | | | | | NSGGTNYAQKFQGRVTMTRDTSISTAY |
| | | | | | | | | | MELSRLRSDDTAVYYCARGDGDYYDSS |
| | | | | | | | | | GYYRPTLYNWLDPWGQGTRVTVSS |
| COVA2- | Ab | SARS-CoV2 | SARS-CoV1 | | SARS-CoV1, | S; RBD | B-cells; SARS-CoV2 | 693 | QVQLQESGPGLVKPSETLSLTCTASGGSI |
| 46 | | | | | SARS-CoV2 | | Human Patient | | SSSSYYWGWIRQPPGKGLEWIGSIYYSG |
| | | | | | | | | | STYYNPSLKSRVTISVDTSKNQFSLKLSS |
| | | | | | | | | | VTAADTAVYYCARHPSGLYQLLNWGQGT |
| | | | | | | | | | LVTVSS |
| COVA2- | Ab | SARS-CoV2 | SARS-CoV1 | | SARS-CoV1, | S; non- | B-cells; SARS-CoV2 | 694 | EVQLVESGGGLVQPGRSLRLSCAASGFT |
| 47 | | | | | SARS-CoV2 | RBD | Human Patient | | FDDYAMHWVRQAPGKGLEWVSGISW |
| | | | | | | | | | NSGSIGYADSVKGRFTISRDSAKNSLYLQ |
| | | | | | | | | | MNSLRAEDTALYYCAKVATYYYDRSGYY |
| | | | | | | | | | YGGALDYWGQGTLVTVSS |
| COVA3- | Ab | SARS-CoV1, | | | SARS-CoV1, | S; non- | B-cells; SARS-CoV2 | 695 | EVQLVESGPGLVKPSETLSLTCTVSGGSI |
| 01 | | SARS-CoV2 | | | SARS-CoV2 | RBD | Human Patient | | SSYYWSWIRQPPGKGLEWIGYIYYSGST |
| | | | | | | | | | NYNPSLKSRVTISVDTSKNQFSLKLSSVT |
| | | | | | | | | | AADTAVYYCARGPAATYYYYMDVWGKG |
| | | | | | | | | | TRVTVSS |
| COVA3- | Ab | SARS-CoV1, | | | SARS-CoV1, | S; non- | B-cells; SARS-CoV2 | 696 | EVQLVESGGGLVQPGGSLRLSCAASGFT |
| 03 | | SARS-CoV2 | | | SARS-CoV2 | RBD | Human Patient | | FSSYAMSWVRQAPGKGLEWVSTISGSG |
| | | | | | | | | | GNTYYADSVKGRFTISRDNSKNTLYLQM |
| | | | | | | | | | NSLRAEDTAVYYCAKEIAVAGCFDYWG |
| | | | | | | | | | QGTLVTVSS |
| COVA3- | Ab | SARS-CoV2 | SARS-CoV1 | | SARS-CoV1, | S; non- | B-cells; SARS-CoV2 | 697 | EVQLVESGGGVVQPGRSLRLSCAASGFT |
| 04 | | | | | SARS-CoV2 | RBD | Human Patient | | FSSYGMHWVRQAPGKGLEWVAVIWY |
| | | | | | | | | | DGSNKYYADSVKGRFTISRDNSKNTLYL |
| | | | | | | | | | QMNSLRAEDTAVYYCARVGSVKSTAGY |
| | | | | | | | | | DFWSGDPFDYWGQGTLVTVSS |
| COVA3- | Ab | SARS-CoV2 | SARS-CoV1 | | SARS-CoV1, | S; RBD | B-cells; SARS-CoV2 | 698 | QVQLVQSGAEVKKPGASVKVSCKVSGY |
| 05 | | | | | SARS-CoV2 | | Human Patient | | TLTELSMHWVRQAPGKGLEWMGGFD |
| | | | | | | | | | PEDGETIYAQKFQGRVTMTEDTSTDTAY |
| | | | | | | | | | MELSSLRSEDTAVYYCATAYSVDTAMVR |
| | | | | | | | | | GVGYWGQGTLVTVSS |
| COVA3- | Ab | SARS-CoV2 | SARS-CoV1 | | SARS-CoV1, | S; RBD | B-cells; SARS-CoV2 | 699 | QVQLVESRAEVKKPGSSVKVSCKASGGT |
| 06 | | | | | SARS-CoV2 | | Human Patient | | FSSYAISWVRQAPGQGLEWMGRIIPILG |
| | | | | | | | | | ITNYAQKFQGRVTITADKSTSTAYMELSS |
| | | | | | | | | | LRSEDTAVYYCARDAPDYYDSSGPTYFD |
| | | | | | | | | | YWGQGTLVTVSS |
| COVA3- | Ab | SARS-CoV2 | SARS-CoV1 | | SARS-CoV1, | S; non- | B-cells; SARS-CoV2 | 700 | QVQLVESGGGLVQPGRSLRLSCAASGFT |
| 07 | | | | | SARS-CoV2 | RBD | Human Patient | | FDDYAMHWVRQAPGKGLEWVSGISW |
| | | | | | | | | | NSGSIGYADSVKGRFTISRDNAKNSLYLQ |
| | | | | | | | | | MNSLRAEDTALYYCAKAEPEVGGYDYY |
| | | | | | | | | | MDVWGKGTMVTVSS |
| COVA3- | Ab | SARS-CoV1, | | | SARS-CoV1, | S; non- | B-cells; SARS-CoV2 | 701 | EVQLVESGPGLVKPSETLSLTCTVSGGSI |
| 08 | | SARS-CoV2 | | | SARS-CoV2 | RBD | Human Patient | | SSYYWSWIRQPPGKGLEWIGYIYYSGST |
| | | | | | | | | | NYNPSLKSRVTISVDTSKNQFSLRLSSVT |
| | | | | | | | | | AADTAVYYCARGPAATYYYYMDVWGK |
| | | | | | | | | | GTMVTVSS |
| COVA3- | Ab | SARS-CoV2 | SARS-CoV1 | | SARS-CoV1, | S; RBD | B-cells; SARS-CoV2 | 702 | EVQLVESGGGLVQPGRSLRLSCAASGFT |
| 09 | | | | | SARS-CoV2 | | Human Patient | | FDDYAMHWVRQAPGKGLEWVSGISW |
| | | | | | | | | | NSGSIGYADSVKGRFTISRDNAKNSLYLQ |
| | | | | | | | | | MNSLRAEDTALYYCAKMGPDPAHDYG |
| | | | | | | | | | RKNDAFDIWGQGTMVTVSS |
| COVA3- | Ab | SARS-CoV1, | | | SARS-CoV1, | S; RBD | B-cells; SARS-CoV2 | 703 | EVQLVESRAEMKKPGESLKISCKGSGYTF |
| 10 | | SARS-CoV2 | | | SARS-CoV2 | | Human Patient | | TNHWIAWVRQMPGKGLEWMGIIYPG |
| | | | | | | | | | DSDTRYSPSFEGQVTISADKSISTAYLQW |
| | | | | | | | | | SSLKASDTAMYYCARRGYTYGADFYGLD |
| | | | | | | | | | VWGQGTRVTVSS |
| CR3022 | Ab | SARS-CoV1, | | SARS-CoV1 | SARS-CoV2 | S; RBD | B-cells; SARS-CoV1 | 704 | QMQLVQSGTEVKKPGESLKISCKGSGY |
| | | SARS-CoV2 | | | | | Human Patient | | GFITYWIGWVRQMPGKGLEWMGIIYP |
| | | | | | | | | | GDSETRYSPSFQGQVTISADKSINTAYLQ |
| | | | | | | | | | WSSLKASDTAIYYCAGGSGISTPMDVW |
| | | | | | | | | | GQGTTVTVSS |
| CV-X1- | Ab | SARS-CoV2 | SARS-CoV1 | SARS-CoV2 | | S; RBD | B-cells; SARS-CoV2 | | ND |
| 126 | | | | | | | Human Patient | | |
| CV-X2- | Ab | SARS-CoV2 | SARS-CoV1 | SARS-CoV2 | | S; RBD | B-cells; SARS-CoV2 | | ND |
| 106 | | | | | | | Human Patient | | |
| CV05-163 | Ab | SARS-CoV2 | SARS-CoV1 | SARS-CoV2 | | S; RBD | B-cells; SARS-CoV2 | | ND |
| | | | | | | | Human Patient | | |
| CV07-200 | Ab | SARS-CoV2 | SARS-CoV1 | SARS-CoV2 | | S; RBD | B-cells; SARS-CoV2 | | ND |
| | | | | | | | Human Patient | | |
| CV07-209 | Ab | SARS-CoV2 | SARS-CoV1 | SARS-CoV2 | | S; RBD | B-cells; SARS-CoV2 | | ND |
| | | | | | | | Human Patient | | |
| CV07-222 | Ab | SARS-CoV2 | SARS-CoV1 | SARS-CoV2 | | S; RBD | B-cells; SARS-CoV2 | | ND |
| | | | | | | | Human Patient | | |
| CV07-250 | Ab | SARS-CoV2 | SARS-CoV1 | SARS-CoV2 | | S; RBD | B-cells; SARS-CoV2 | | ND |
| | | | | | | | Human Patient | | |
| CV07-255 | Ab | SARS-CoV2 | SARS-CoV1 | SARS-CoV2 | | S; RBD | B-cells; SARS-CoV2 | | ND |
| | | | | | | | Human Patient | | |
| CV07-262 | Ab | SARS-CoV2 | SARS-CoV1 | SARS-CoV2 | | S; RBD | B-cells; SARS-CoV2 | | ND |
| | | | | | | | Human Patient | | |
| CV07-270 | Ab | SARS-CoV2 | SARS-CoV1 | SARS-CoV2 | | S; RBD | B-cells; SARS-CoV2 | | ND |
| | | | | | | | Human Patient | | |
| CV07-283 | Ab | SARS-CoV2 | SARS-CoV1 | SARS-CoV2 | | S; RBD | B-cells; SARS-CoV2 | | ND |
| | | | | | | | Human Patient | | |
| CV07-287 | Ab | SARS-CoV2 | SARS-CoV1 | SARS-CoV2 | | S; RBD | B-cells; SARS-CoV2 | | ND |
| | | | | | | | Human Patient | | |
| CV07-315 | Ab | SARS-CoV2 | SARS-CoV1 | SARS-CoV2 | | S; RBD | B-cells; SARS-CoV2 | | ND |
| | | | | | | | Human Patient | | |
| CV1 | Ab | SARS-CoV1, | | SARS-CoV2 | | S; non- | B-cells; SARS-CoV2 | 705 | QVQLQESGPGLVKPSETLSLTCTVSGYSI |
| | | SARS-CoV2 | | | | RBD | Human Patient | | SSGYYWGWIRQPPGKGLEWIGSIYHSG |
| | | | | | | | | | STYYNPSLKSRVTISVDTSKNQFSLKLSS |
| | | | | | | | | | VTAADTAVYYCARTPLSLRLRYNWYFDLW |
| | | | | | | | | | GRGTLVTVSS |
| CV10 | Ab | SARS-CoV1, | | | SARS-CoV2 | S; non- | B-cells; SARS-CoV2 | 706 | QVQLQESGPGLVKPSETLSLTCNVSGGSI |
| | | SARS-CoV2 | | | | RBD | Human Patient | | SSYYWSWIRQPPGKGLEWIGYIYYSGST |
| | | | | | | | | | NYNPSLKSRVTISVDTSKNQFSLKLSSVT |
| | | | | | | | | | AADTAVYYCARGFDYWGQGTLVTVSS |
| CV11 | Ab | SARS-CoV1, | | | SARS-CoV2 | S; non- | B-cells; SARS-CoV2 | 707 | QVQLQESGPGLVKPSQTLSLTCTVSGGSI |
| | | SARS-CoV2 | | | | RBD | Human Patient | | SSGGYYWSWIRQHPGKGLEWIGYIYYS |
| | | | | | | | | | GSTYYNPSLKSRVTISVDTSKNQFSLKLS |
| | | | | | | | | | SVTAADTAVYYCARETTGHFDYWGQGTL |
| | | | | | | | | | VTVSS |
| CV12 | Ab | SARS-CoV2 | SARS-CoV1 | | SARS-CoV2 | S; non- | B-cells; SARS-CoV2 | 708 | QVQLVESGGGVVQPGRSLRLSCAASGF |
| | | | | | | RBD | Human Patient | | NLSNYAMHWVRQASGKGLEWVSLISY |
| | | | | | | | | | DGSVKYYTDSVKGRFTVSGDNSKNTLFL |
| | | | | | | | | | QMNSLRPDDSALYYCVRGGVSGPNSFD |
| | | | | | | | | | MWGQGTTVTVSS |
| CV13 | Ab | SARS-CoV1, | | | SARS-CoV2 | S; non- | B-cells; SARS-CoV2 | 709 | QVQLVQSGSELKKPGASVKLSCQASGYS |
| | | SARS-CoV2 | | | | RBD | Human Patient | | FTNHAMNWVRQAPGQGLEWMGWIN |
| | | | | | | | | | TNTGNPTYAQGFTGRFVFSLDTSVSTTYL |
| | | | | | | | | | HISSLKAEDTAVYYCARASARPGVATNL |
| | | | | | | | | | DFWGQGTLVVVSS |
| CV15 | Ab | SARS-CoV2 | SARS-CoV1 | | SARS-CoV2 | S; non- | B-cells; SARS-CoV2 | 710 | EVQLVESGGGLVQPGGSLRLSCAASGFT |
| | | | | | | RBD | Human Patient | | FSSYWMSWVRQAPGKGLEWVANIKQ |
| | | | | | | | | | DGSEKYYVDSVKGRFTISRDNAKNSLYL |
| | | | | | | | | | QMNSLRAEDTAVYYCARDFNSYQLLWY |
| | | | | | | | | | YYYGMDVWGQGTTVTVSS |
| CV16 | Ab | SARS-CoV2 | SARS-CoV1 | | SARS-CoV2 | S; non- | B-cells; SARS-CoV2 | 711 | EVQLVQSGAEVKKPGESLKISCKGSGYSF |
| | | | | | | RBD | Human Patient | | TSYWIGWVRQMPGKGLEWMGIIYPGD |
| | | | | | | | | | SDTRYSPSFQGQVTISADKSISTAYLQWS |
| | | | | | | | | | SLKASDTAMYYCARQSSFYSSGWYSYG |
| | | | | | | | | | MDVWGQGTTVTVSS |
| CV17 | Ab | SARS-CoV2 | SARS-CoV1 | | SARS-CoV2 | S; non- | B-cells; SARS-CoV2 | 712 | QVQLVQSGAEVKKPGASVKVSCKASGY |
| | | | | | | RBD | Human Patient | | TFTGYYMHWVRQAPGQGLEWMGWI |
| | | | | | | | | | NPNSGGTNYAQKFQGRVTMTRDTSIST |
| | | | | | | | | | AYMELSRLRSDDTAVYYCARVDYGSGSY |
| | | | | | | | | | GWGWFDPWGQGTLVTVSS |
| CV18 | Ab | SARS-CoV1, | | | SARS-CoV2 | S; non- | B-cells; SARS-CoV2 | 713 | QVQLVQSGAEVKKPGASVKVSCKVSGY |
| | | SARS-CoV2 | | | | RBD | Human Patient | | TLTELSMHWVRQAPGKGLEWMGGFD |
| | | | | | | | | | PEDGETIYAQKFQGRVTMTEDTSTDTAY |
| | | | | | | | | | MELSSLRSEDTAVYYCATTSPIVGAITWF |
| | | | | | | | | | DPWGQGTLVTVSS |
| CV19 | Ab | SARS-CoV2 | SARS-CoV1 | | SARS-CoV2 | S; non- | B-cells; SARS-CoV2 | 714 | QVQLVQSGAEVKKPGASVKVSCKASGY |
| | | | | | | RBD | Human Patient | | TFTGYYMHWVRQAPGQGLEWMGWI |
| | | | | | | | | | NPNSGGTNYAQKFQGRVTMTRDTSIST |
| | | | | | | | | | AYMELSRLRSDDTAVYYCAREYYYDSSVY |
| | | | | | | | | | PYYYYAMDVWGQGTTVTVSS |
| CV2 | Ab | SARS-CoV2 | SARS-CoV1 | | SARS-CoV2 | S; non- | B-cells; SARS-CoV2 | 715 | QVQLVESGGGVVQPGRSLRLSCAASGF |
| | | | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARVRGSYYLFDY |
| | | | | | | | | | WGQGTLVTVSS |
| CV21 | Ab | SARS-CoV2 | SARS-CoV1 | | SARS-CoV2 | S; non- | B-cells; SARS-CoV2 | 716 | EVQLVESGGGLVKPGGSLRLSCAASGFT |
| | | | | | | RBD | Human Patient | | FSNAWMSWVRQAPGKGLEWVGRIKSK |
| | | | | | | | | | TDGGTTDYAAPVKGRFTISRDDSKNTLY |
| | | | | | | | | | LQMNSLKTEDTAVYYCTTDRVYDYIWGS |
| | | | | | | | | | YRYLDYWGQGTLVTVSS |
| CV22 | Ab | SARS-CoV1, | | | SARS-CoV2 | S; non- | B-cells; SARS-CoV2 | 717 | EVQLVESGGGLVKPGGSLRLSCAASGFT |
| | | SARS-CoV2 | | | | RBD | Human Patient | | FSSYSMNWVRQAPGKGLEWVSSISSSSS |
| | | | | | | | | | YIYYADSVKGRFTISRDNAKNSLYLQMNS |
| | | | | | | | | | LRAEDTAVYYCARDRESYDILTGYSMEG |
| | | | | | | | | | CFDYWGQGTLVTVSS |
| CV23 | Ab | SARS-CoV2 | SARS-CoV1 | | SARS-CoV2 | S; non- | B-cells; SARS-CoV2 | 718 | QVQLVQSGAEVKKPGASVKVSCKASGY |
| | | | | | | RBD | Human Patient | | TFTSYAMHWVRQAPGQRLEWMGWIN |
| | | | | | | | | | AGNGNTKYSQKFQGRVTITRDTSASTAY |
| | | | | | | | | | MELSSLRSEDTAVYYCARVWGYCSGGS |
| | | | | | | | | | CYVDAFDIWGQGTMVTVSS |
| CV24 | Ab | SARS-CoV2 | SARS-CoV1 | | SARS-CoV2 | S; non- | B-cells; SARS-CoV2 | 719 | QVQLVQSGAEVKKPGASVKVSCKVSGY |
| | | | | | | RBD | Human Patient | | TLTELSMHWVRQAPGKGLEWMGGFD |
| | | | | | | | | | PEDGETIYAQKFQGRVTMTEDTSTDTAY |
| | | | | | | | | | MELSSLRSEDTAVYYCATAPPYSPPSSWF |
| | | | | | | | | | DPWGQGTLVTVSS |
| CV25 | Ab | SARS-CoV2 | SARS-CoV1 | | SARS-CoV2 | S; non- | B-cells; SARS-CoV2 | 720 | QVQLQESGPGLVKPSQTLSLTCTVSGGSI |
| | | | | | | RBD | Human Patient | | SSGDYYWSWIRQPPGKGLEWIGYIYYSG |
| | | | | | | | | | STYYNPSLKSRVTISVDTSKNQFSLKLSS |
| | | | | | | | | | VTAADTAVYYCARDHHYDFWSGYSSYYYY |
| | | | | | | | | | GMDVWGQGTTVTVSS |
| CV26 | Ab | SARS-CoV1, | | | SARS-CoV2 | S; non- | B-cells; SARS-CoV2 | 721 | QVQLVESGGGVVQPGRSLRLSCAASGF |
| | | SARS-CoV2 | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARDEAYYDILTGY |
| | | | | | | | | | INAPKNYYYYGMDVWGQGTTVTVSS |
| CV27 | Ab | SARS-CoV2 | SARS-CoV1 | | SARS-CoV2 | S; non- | B-cells; SARS-CoV2 | 722 | QVQLVESGGGVVQPGRSLRLSCAASGF |
| | | | | | | RBD | Human Patient | | TFSSYAMHWVRQAPGKGLEWVALISYD |
| | | | | | | | | | GSNKYYADSVKGRFTISRDNSKNTLYLQ |
| | | | | | | | | | MNSLRAEDTAVYYCARSFGGSYYYGMD |
| | | | | | | | | | VWGQGTTVTVSS |
| CV3 | Ab | SARS-CoV1, | | | SARS-CoV2 | S; non- | B-cells; SARS-CoV2 | 723 | QVQLVQSGSELKKPGASVKLSCQASGYS |
| | | SARS-CoV2 | | | | RBD | Human Patient | | FTNHAMNWVRQAPGQGLEWMGWIN |
| | | | | | | | | | TNTGNPTYAQGFTGRFVFSLDTSVSTTYL |
| | | | | | | | | | HISSLKAEDTAVYYCARASARPGVATNL |
| | | | | | | | | | DFWGQGTLVVVSS |
| CV30 | Ab | SARS-CoV2 | SARS-CoV1 | SARS-CoV2 | | S; RBD | B-cells; SARS-CoV2 | 724 | EVQLVESGGGLIQPGGSLRLSCAASGVI |
| | | | | | | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAEDTAVYYCARDLDVSGGMDVW |
| | | | | | | | | | GQGTTVTVSS |
| CV31 | Ab | SARS-CoV1, | | | SARS-CoV2 | S; non- | B-cells; SARS-CoV2 | 725 | QVQLVQSGAEVKKPGASVKVSCKVSGY |
| | | SARS-CoV2 | | | | RBD | Human Patient | | TLTELSMHWVRQAPGKGLEWMGGFD |
| | | | | | | | | | PEDGETIYAQKFQGRVTMTEDTSTDTAY |
| | | | | | | | | | MELSSLRSEDTAVYYCATAPPYSPPSSWF |
| | | | | | | | | | DPWGQGTLVTVSS |
| CV32 | Ab | SARS-CoV2 | SARS-CoV1 | | SARS-CoV2 | S; non- | B-cells; SARS-CoV2 | 726 | QVQLVQSGAEVKKPGASVKVSCKASGY |
| | | | | | | RBD | Human Patient | | TFTGYYMHWVRQAPGQGLEWMGWI |
| | | | | | | | | | NPNSDVTNYAQKFQGRVTMTRDTSIST |
| | | | | | | | | | AYMELSRLRSDDTAVYYCAREARDYYGS |
| | | | | | | | | | GSLDYWGQGTLVTVSS |
| CV33 | Ab | SARS-CoV2 | SARS-CoV1 | | SARS-CoV2 | S; non- | B-cells; SARS-CoV2 | 727 | QVQLVQSGAEVKKPGASVKVSCKASGY |
| | | | | | | RBD | Human Patient | | TFTSYDISWVRQAPGQGLEWMGWISA |
| | | | | | | | | | YNGNTNYAQKLQGRVTMTTDTSTSTAY |
| | | | | | | | | | MELRSLRSDDTAVYYCARDSVAGIYYYY |
| | | | | | | | | | GMDVWGQGTTVTVSS |
| CV34 | Ab | SARS-CoV2 | SARS-CoV1 | | SARS-CoV2 | S; non- | B-cells; SARS-CoV2 | 728 | QVQLVESGGGVVQPGRSLRLSCAASGF |
| | | | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARSYGGSYYYGM |
| | | | | | | | | | DVWGQGTTVTVSS |
| CV35 | Ab | SARS-CoV2 | SARS-CoV1 | | SARS-CoV2 | S; non- | B-cells; SARS-CoV2 | 729 | QVQLQESGPGLVKPSETLSLTCTVSGYSI |
| | | | | | | RBD | Human Patient | | SSGYYWGWIRQPPGKGLEWIGSIYHSG |
| | | | | | | | | | STYYNPSLKSRVTISVDTSKNQFSLKLSS |
| | | | | | | | | | VTAADTAVYYCARTPLSLRLRYNWYFDLW |
| | | | | | | | | | GRGTLVTVSS |
| CV36 | Ab | SARS-CoV2 | SARS-CoV1 | | SARS-CoV2 | S; non- | B-cells; SARS-CoV2 | 730 | QVQLVQSGAEVKKPGASVKVSCKASGY |
| | | | | | | RBD | Human Patient | | TFTGYYMHWVRQAPGQGLEWMGWI |
| | | | | | | | | | NPNSGGTNYVQKFQGRVTMTRDTSIST |
| | | | | | | | | | AYMELNRLRSDDTAVYFCARDLTTTAGT |
| | | | | | | | | | DYYYGMDVWGQGTTVTVSS |
| CV37 | Ab | SARS-CoV1, | | | SARS-CoV2 | S; non- | B-cells; SARS-CoV2 | 731 | QVQLVQSGAEVKKPGASVKVSCKASGY |
| | | SARS-CoV2 | | | | RBD | Human Patient | | TFTSYGISWVRQAPGQGLEWMGWISA |
| | | | | | | | | | YNGNTNYAQKLQGRVTMTTDTSTSTAY |
| | | | | | | | | | MELRSLRSDDTAVYYCARARVAYDYIW |
| | | | | | | | | | GSYRYKAFDYWGQGTLVTVSS |
| CV38 | Ab | SARS-CoV1, | | | SARS-CoV2 | S; non- | B-cells; SARS-CoV2 | 732 | QVQLVESGGGVVQPGRSLRLSCAASGF |
| | | SARS-CoV2 | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARAQTAHYSSSF |
| | | | | | | | | | DYWGQGTLVTVSS |
| CV38-113 | Ab | SARS-CoV2 | SARS-CoV1 | SARS-CoV2 | | S; RBD | B-cells; SARS-CoV2 | | ND |
| | | | | | | | Human Patient | | |
| CV38-139 | Ab | SARS-CoV2 | SARS-CoV1 | SARS-CoV2 | | S; RBD | B-cells; SARS-CoV2 | | ND |
| | | | | | | | Human Patient | | |
| CV38-142 | Ab | SARS-CoV1, | | SARS-CoV2 | | S; RBD | B-cells; SARS-CoV2 | | ND |
| | | SARS-CoV2 | | | | | Human Patient | | |
| CV38-183 | Ab | SARS-CoV2 | SARS-CoV1 | SARS-CoV2 | | S; RBD | B-cells; SARS-CoV2 | | ND |
| | | | | | | | Human Patient | | |
| CV38-221 | Ab | SARS-CoV2 | SARS-CoV1 | SARS-CoV2 | | S; RBD | B-cells; SARS-CoV2 | | ND |
| | | | | | | | Human Patient | | |
| CV39 | Ab | SARS-CoV2 | SARS-CoV1 | | SARS-CoV2 | S; non- | B-cells; SARS-CoV2 | 733 | QVQLVESGGGVVQPGRSMRLSCAASG |
| | | | | | | RBD | Human Patient | | FNLSSYAMHWVRQASGKGLEWVSLISY |
| | | | | | | | | | DGSIKYYADSVKGRFTVSGDNSKNTLFL |
| | | | | | | | | | QMSSLRADDSALYYCVRGGVSGPNAFD |
| | | | | | | | | | IWGQGTTVTVSS |
| CV4 | Ab | SARS-CoV1, | | | SARS-CoV2 | S; non- | B-cells; SARS-CoV2 | 734 | QVQLVESGGGVVQPGRSLRLSCAASGF |
| | | SARS-CoV2 | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARSISGSYLGAFD |
| | | | | | | | | | YWGQGTLVTVSS |
| CV40 | Ab | SARS-CoV1, | | | SARS-CoV2 | S; non- | B-cells; SARS-CoV2 | 735 | QVQLVQSGAEVKKPGASVKVSCKASGY |
| | | SARS-CoV2 | | | | RBD | Human Patient | | TFSSYGISWVRQDPGQGLEWMGWISA |
| | | | | | | | | | YNGNTNYAQKLQGRVTMTTDTSTSTAY |
| | | | | | | | | | MELRSLRSDDTAVYYCARVGLWWLGH |
| | | | | | | | | | PDAFDIWGQGTMVTVSS |
| CV41 | Ab | SARS-CoV2 | SARS-CoV1 | | SARS-CoV2 | S; non- | B-cells; SARS-CoV2 | 736 | QVQLVESGGGVVQPGRSLRLSCAASGF |
| | | | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARTKGGSYFAPF |
| | | | | | | | | | DYWGQGTLVTVSS |
| CV42 | Ab | SARS-CoV2 | SARS-CoV1 | | SARS-CoV2 | S; non- | B-cells; SARS-CoV2 | 737 | QVQLVQSGAEVKKPGASVKVSCKASGY |
| | | | | | | RBD | Human Patient | | TFTSYGISWVRQAPGQGLEWMGWISA |
| | | | | | | | | | YNGNTNYAQKVQGRVTMTTDTSTSTAY |
| | | | | | | | | | MELRSLRSDDTAVYYCARDRGYAATFG |
| | | | | | | | | | VFDYWGQGTLVTVSS |
| CV43 | Ab | SARS-CoV2 | SARS-CoV1 | | SARS-CoV2 | S; RBD | B-cells; SARS-CoV2 | 738 | QVQLVESGGGVVQPGRSLRLSCAASGF |
| | | | | | | | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARVTVVHFDYW |
| | | | | | | | | | GQGTLVTVSS |
| CV44 | Ab | SARS-CoV2 | SARS-CoV1 | | SARS-CoV2 | S; non- | B-cells; SARS-CoV2 | 739 | QVQLVQSGAEVKKPGASVKVSCKASGY |
| | | | | | | RBD | Human Patient | | TFTSYYMHWVRQAPGQGLEWMGIINP |
| | | | | | | | | | SGGSTSYAQKFQGRVTMTRDTSTSTVY |
| | | | | | | | | | MELSSLRSEDTAVYYCARDLTSTSSSPYS |
| | | | | | | | | | YYYGMDVWGQGTTVTVSS |
| CV45 | Ab | SARS-CoV1, | | | SARS-CoV2 | S; non- | B-cells; SARS-CoV2 | 740 | QVQLVQSGAEVKKPGASVKVSCKASGY |
| | | SARS-CoV2 | | | | RBD | Human Patient | | TFTSYGISWVRQAPGQGLEWMGWISA |
| | | | | | | | | | YNGNTNYAQKLQGRVTMTTDTSTSTAY |
| | | | | | | | | | MELRSLRSDDTAVYYCARVTVEAIFGVVI |
| | | | | | | | | | LPLKNWFDPWGQGTLVTVSS |
| CV46 | Ab | SARS-CoV2 | SARS-CoV1 | | SARS-CoV2 | S; non- | B-cells; SARS-CoV2 | 741 | QVQLVESGGGVVQPGRSLRLSCAASGF |
| | | | | | | RBD | Human Patient | | NLSNYAMHWVRQASGKGLEWVSLISY |
| | | | | | | | | | DGSIKYYTDSVKGRFTVSGDNSKNTLFLQ |
| | | | | | | | | | MNSLRPDDSALYYCVRGGVSGPNSFD |
| | | | | | | | | | MWGQGTTVTVSS |
| CV47 | Ab | SARS-CoV2 | SARS-CoV1 | | SARS-CoV2 | S; non- | B-cells; SARS-CoV2 | 742 | QVQLVQSGAEVKKPGASVKVSCKASGY |
| | | | | | | RBD | Human Patient | | TFTSYGISWVRQAPGQGLEWMGWISA |
| | | | | | | | | | YSGNTNYAQKLQGRVTMTTDTSTSTAY |
| | | | | | | | | | MEVRSLRSDDTAVYYCARVGLWWLGH |
| | | | | | | | | | PDVFDIWGQGTMVTVSS |
| CV48 | Ab | SARS-CoV2 | | | SARS-CoV2 | S; non- | B-cells; SARS-CoV2 | 743 | QVQLVQSGAEVKKPGSSVKVSCKASGG |
| | | | | | | RBD | Human Patient | | TFSSYTINWVRQAPGQGLEWMGRIIPIL |
| | | | | | | | | | GIADYAQKFQGRVTITADKSTSTAYMEL |
| | | | | | | | | | SSLRSEDTAVYYCARDLVEDTAMVTGAA |
| | | | | | | | | | AGTWGQGTLVTVSS |
| CV5 | Ab | SARS-CoV2 | SARS-CoV1 | | SARS-CoV2 | S; RBD | B-cells; SARS-CoV2 | 744 | QVQLVQSGAEVKKPGASVKVTCKASGY |
| | | | | | | | Human Patient | | TFTSYYLHWVRQAPGQGLEWMGIINPS |
| | | | | | | | | | GGTTSYAQKFQGRVTMTRDTSTSTVYM |
| | | | | | | | | | ELSSLRSEDTAVYYCARAGRRYSSSDDGA |
| | | | | | | | | | FDIWGQGTMVTVSS |
| CV50 | Ab | SARS-CoV2 | | | SARS-CoV2 | S; non- | B-cells; SARS-CoV2 | 745 | QVQLVESGGGVVQPGRSLRLSCAASGF |
| | | | | | | RBD | Human Patient | | TFSSYGMHWVRQAPGKGLEWVAVIWY |
| | | | | | | | | | DGSNKYYADSVKGRFTISRDNSKNTLYL |
| | | | | | | | | | QMNSLRAEDTAVYYCARDIMFGDDWL |
| | | | | | | | | | QKQPDYWGQGTLVTVSS |
| CV7 | Ab | SARS-CoV2 | SARS-CoV1 | | SARS-CoV2 | S; non- | B-cells; SARS-CoV2 | 746 | QVQLVESGGGVVQPGRSLRLSCAASGF |
| | | | | | | RBD | Human Patient | | TFSSYAMHWVRQAPGKGLEWVAVISY |
| | | | | | | | | | DGSNKYYADSVKGRFTISRDNSKNTLYL |
| | | | | | | | | | QMNSLRAEDTAVYYCARSISGSYLGAFD |
| | | | | | | | | | YWGQGTLVTVSS |
| CV8 | Ab | SARS-CoV1, | | | SARS-CoV2 | S; non- | B-cells; SARS-CoV2 | 747 | QVQLVQSGAEVKKPGASVKVSCKASGY |
| | | SARS-CoV2 | | | | RBD | Human Patient | | TFTSYGISWVRQAPGQGLEWMGWISA |
| | | | | | | | | | YNGNTNYAQKLQGRVTMTTDTSTSTAY |
| | | | | | | | | | MELRSLRSDDTAVYYCARLVPTWASYYD |
| | | | | | | | | | FWSGYPGGYGMDVWGQGTTVTVSS |
| CV9 | Ab | SARS-CoV1, | | | SARS-CoV2 | S; non- | B-cells; SARS-CoV2 | 748 | QLQLQESGPGLVKPSETLSLTCTVSGGSI |
| | | SARS-CoV2 | | | | RBD | Human Patient | | SSSSYYWGWIRQPPGKGLEWIGSIYYSG |
| | | | | | | | | | STYYNPSLKSRVTISVDTSKNQFSLKLSS |
| | | | | | | | | | VTAADTAVYYCATHIVVVTATPNWYFDL |
| | | | | | | | | | WGRGTLVTVSS |
| EY6A | Ab | SARS-CoV2 | | SARS-CoV2 | | S; RBD | B-cells; SARS-CoV2 | 749 | EVQLVESGGGVVQPGRSLRLSCAASAFT |
| | | | | | | | Human Patient | | FSSYDMHWVRQAPGKGLEWVAVISYD |
| | | | | | | | | | GSNKYYADSVKGRFTISRDNSKNTLYLQ |
| | | | | | | | | | MNSLRAEDTAVYYCAKDGGKLWVYYFD |
| | | | | | | | | | YWGQGTLVTVSS |
| Fab 2-4 | Ab | SARS-CoV2 | | SARS-CoV2 | | S; RBD | B-cells; SARS-CoV2 | 750 | QVQLVQSGAEVKKPGASVKVSCKASGY |
| | | | | | | | Human Patient | | TFTGYYMHWVRQAPGQGLEWMGWI |
| | | | | | | | | | NPNSGGTNYTQMFQGRVTMTRDTSIST |
| | | | | | | | | | AYMEVSRLRSDDTAVYYCARDRSWAVV |
| | | | | | | | | | YYYMDVWGKGTTVTVSS |
| FnC1t1p2_ | Ab | SARS-CoV2 | | SARS-CoV2 | | S; SInon- | B-cells; SARS-CoV2 | 751 | QVQLVQSGAEVKKPGASVKVSCKASGY |
| A5 | | | | | | RBD | Human Patient | | TFTSYDINWVRQATGQGLEWMGWMN |
| | | | | | | | | | PNSGNTGYAQKFQGRVTMTRNTSISTA |
| | | | | | | | | | YMELSSLRSEDTAVYYCARATTDCSSTSC |
| | | | | | | | | | WSLDFWSGYYTGGREKIFDWGQGTLVT |
| | | | | | | | | | VSS |
| FnC1t2p1_ | Ab | SARS-CoV2 | | SARS-CoV2 | | S; RBD | B-cells; SARS-CoV2 | 752 | QVQLVQSGSELKKPGASVKVSCKASGYT |
| D4 | | | | | | | Human Patient | | FLRFAMNWLRQAPGQGLEWMGWIDT |
| | | | | | | | | | NTGTPTYAQGFTGRFVFSLDTSVSTAYL |
| | | | | | | | | | QISSLKAEDTAVYYCARSLRGANLVPWG |
| | | | | | | | | | QGTLVTVSS |
| FnC1t2p1_ | Ab | SARS-CoV2 | | SARS-CoV2 | | S; RBD | B-cells; SARS-CoV2 | 753 | QVQLVQSGSELKKPGASVKVSCKASGYT |
| G5 | | | | | | | Human Patient | | FLRFAMNWLRQAPGQGLEWMGWIDT |
| | | | | | | | | | NTGTPTYAQGFTGRFVFSLDTSVSTAYL |
| | | | | | | | | | QISSLKAEDTAVYYCARSLRGANLVPWG |
| | | | | | | | | | QGTLVTVSS |
| H014 | Ab | SARS-CoV1, | | SARS-CoV1, | | S; RBD | Immunised | | ND |
| | | SARS-CoV2 | | SARS-CoV2 | | | Humanised (hACE2) | | |
| | | | | | | | Mouse | | |
| H11-D4 | Nb | SARS-CoV2 | | SARS-CoV2 | | S; RBD | Phage Display | 754 | QVQLVESGGGLMQAGGSLRLSCAVSGR |
| | | | | | | | Library (Nanobody, | | TFSTAAMGWFRQAPGKEREFVAAIRWS |
| | | | | | | | non-immune) | | GGSAYYADSVKGRFTISRDKAKNTVYLQ |
| | | | | | | | | | MNSLKYEDTAVYYCARTENVRSLLSDYA |
| | | | | | | | | | TWPYDYWGQGTQVTVSS |
| H11-H4 | Nb | SARS-CoV2 | | SARS-CoV2 | | S; RBD | Phage Display | 755 | QVQLVESGGGLMQAGGSLRLSCAVSGR |
| | | | | | | | Library (Nanobody, | | TFSTAAMGWFRQAPGKEREFVAAIRWS |
| | | | | | | | non-immune) | | GGSAYYADSVKGRFTISRDKAKNTVYLQ |
| | | | | | | | | | MNSLKYEDTAVYYCAQTHYVSYLLSDYA |
| | | | | | | | | | TWPYDYWGQGTQVTVSS |
| H4 | Ab | SARS-CoV2 | | SARS-CoV2 | | S; RBD | B-cells; SARS-CoV2 | 756 | QVQLVQSGAEVKKPGASVKVSCKASGY |
| | | | | | | | Human Patient | | TFTGYYMHWVRQAPGQGLEWMGRIN |
| | | | | | | | | | PNSGGTNYAQKFQGRVTMTRDTSISTA |
| | | | | | | | | | YMELSRLRSDDTAVYYCARVPYCSSTSCH |
| | | | | | | | | | RDWYFDLWGRGTLVTVSS |
| HbnC2t1p2_ | Ab | SARS-CoV2 | | SARS-CoV2 | | S; RBD | B-cells; SARS-CoV2 | 757 | QVQLVESGGGVVQPGRSLRLSCAASGF |
| D9 | | | | | | | Human Patient | | TFSSYGMHWVRQAPGKGLEWVAVIWY |
| | | | | | | | | | DGRNKYYVDSVKGRFTISRDNSKNTLYL |
| | | | | | | | | | QISSLRAEDTAVYYCARAARRPVVTDTM |
| | | | | | | | | | AYYMDVWGKGTTVTVSS |
| HbnC3t1p1_ | Ab | SARS-CoV2 | | SARS-CoV2 | | S; RBD | B-cells; SARS-CoV2 | 758 | QMQLVQSGPEVKKPGTSVKVSCKASGF |
| C6 | | | | | | | Human Patient | | TFTSSAVQWVRQARGQRLEWIGWIVV |
| | | | | | | | | | GSGNTNYAQKFQERVTITRDMSTSTAY |
| | | | | | | | | | MELSSLRSEDTAVYYCAAPHCSSTICYDG |
| | | | | | | | | | FDIWGQGTMVTVSS |
| HbnC3t1p1_ | Ab | SARS-CoV2 | | SARS-CoV2 | | S; RBD | B-cells; SARS-CoV2 | 759 | QVQLVESGGGVVQPGRSLRLSCAATGF |
| F4 | | | | | | | Human Patient | | TFRRYGMHWVRQAPGKGLEWVAGILF |
| | | | | | | | | | DGSNKYYVDSVKGRFTISRDSSRNTLYLQ |
| | | | | | | | | | LNSLRREDTAVYYCAKGGDYEWELLES |
| | | | | | | | | | WGQGTLVTVSS |
| HbnC3t1p1_ | Ab | SARS-CoV2 | | SARS-CoV2 | | S; RBD | B-cells; SARS-CoV2 | 760 | EVQLVESGGGLVQPGGSLRLSCAASGFT |
| G4 | | | | | | | Human Patient | | VSSNYMSWVRQAPGKGLEWVSVIYSG |
| | | | | | | | | | GSTFYADSVKGRFTISRDNSKNTLYLQM |
| | | | | | | | | | NSLRAEDTAVYYCARDFGDFFFDYWGQ |
| | | | | | | | | | GTLVTVSS |
| HbnC3t1p2_ | Ab | SARS-CoV2 | | SARS-CoV2 | | S; RBD | B-cells; SARS-CoV2 | 761 | EVQLVESGGGLVQPGGSLRLSCAASGFI |
| B10 | | | | | | | Human Patient | | VSSNYMSWVRQAPGKGLEWVSVIYSG |
| | | | | | | | | | GSTFYADSVKGRFTISRDNSKNTLYLQM |
| | | | | | | | | | NSLRAEDTAVYYCARDYGDYFFDYWGQ |
| | | | | | | | | | GTLVTVSS |
| HbnC3t1p2_ | Ab | SARS-CoV2 | | SARS-CoV2 | | S; RBD | B-cells; SARS-CoV2 | 762 | QMQLVQSGPEVKKPGTSVKVSCKASGF |
| C6 | | | | | | | Human Patient | | TFSSSAVQWVRQARGQRLEWIGWIVV |
| | | | | | | | | | GSGNTNYAQKFQGRVTITRDMSTRTAY |
| | | | | | | | | | MELSSLRSEDTAMYYCAAPYCSSTRCYD |
| | | | | | | | | | AFDIWGQGTMVTVSS |
| HbnC4t1p1_ | Ab | SARS-CoV2 | | SARS-CoV2 | | S; RBD | B-cells; SARS-CoV2 | 763 | EVQLVESGGGLVQPGRSLRLSCAASGFT |
| D5 | | | | | | | Human Patient | | FDDYAMHWVRQAPGKGLEWVSGISW |
| | | | | | | | | | NSGSIGYADSVKGRFTISRDNAKNSLYLQ |
| | | | | | | | | | MNSLRAEDTGLYYCAKDINYDSGGYHK |
| | | | | | | | | | NYFDYWGQGTLVTVSS |
| Ju et al., | Ab | SARS-CoV2 | | SARS-CoV2 | SARS-CoV2 | S; Various | B-cells; SARS-CoV2 | | 205 |
| 2020 | | | | | | | Human Patient | | |
| Kim et | Ab | SARS-CoV2 | | SARS-CoV2 | | S; RBD | B-cells; SARS-CoV2 | | Various |
| al., 2020 | | | | | | | Human Patient | | |
| LR1 | Nb | SARS-CoV2 | | | | S; RBD | Phage Display | 764 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| LR11 | Nb | SARS-CoV2 | | | | S; RBD | Phage Display | 765 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| LR15 | Nb | SARS-CoV2 | | | | S; RBD | Phage Display | 766 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| LR16 | Nb | SARS-CoV2 | | | | S; RBD | Phage Display | 767 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| LR2 | Nb | SARS-CoV2 | | | | S; RBD | Phage Display | 768 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| LR3 | Nb | SARS-CoV2 | | | | S; RBD | Phage Display | 769 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| LR5 | Nb | SARS-CoV2 | | | | S; RBD | Phage Display | 770 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| LR6 | Nb | SARS-CoV2 | | | | S; RBD | Phage Display | 771 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| LR7 | Nb | SARS-CoV2 | | | | S; RBD | Phage Display | 772 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| LR8 | Nb | SARS-CoV2 | | | | S; RBD | Phage Display | 773 | QVQLVESGGGSVQAGGSLRLSCAASGSI |
| | | | | | | | Library (Nanobody, | | SSITYLGWFRQAPGKEREGVAALITDSG |
| | | | | | | | non-immune) | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-1 | Ab | SARS-CoV1, | 229E, HKU1, | | | S; Unk | B-cells; SARS-CoV1 | 774 | QVQLVESGGGSVQAGGSLRLSCAASGSI |
| | | SARS-CoV2 | NL63, OC43 | | | | Human Patient | | SSITYLGWFRQAPGKEREGVAALITDSG |
| | | | | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-10 | Ab | SARS-CoV1, | 229E, HKU1, | | | S; Unk | B-cells; SARS-CoV1 | 775 | QVQLVESGGGSVQAGGSLRLSCAASGSI |
| | | SARS-CoV2 | NL63, OC43 | | | | Human Patient | | SSITYLGWFRQAPGKEREGVAALITDSG |
| | | | | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-100 | Ab | SARS-CoV2 | 229E, HKU1, | | | S; Unk | B-cells; SARS-CoV1 | 776 | QVQLVESGGGSVQAGGSLRLSCAASGSI |
| | | (weak) | NL63, OC43, | | | | Human Patient | | SSITYLGWFRQAPGKEREGVAALITDSG |
| | | | SARS-CoV1 | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-101 | Ab | SARS-CoV2 | 229E, HKU1, | | | S; Unk | B-cells; SARS-CoV1 | 777 | QVQLVESGGGSVQAGGSLRLSCAASGSI |
| | | (weak) | NL63, OC43, | | | | Human Patient | | SSITYLGWFRQAPGKEREGVAALITDSG |
| | | | SARS-CoV1 | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-102 | Ab | SARS-CoV2 | 229E, HKU1, | | | S; Unk | B-cells; SARS-CoV1 | 778 | QVQLVESGGGSVQAGGSLRLSCAASGSI |
| | | (weak) | NL63, OC43, | | | | Human Patient | | SSITYLGWFRQAPGKEREGVAALITDSG |
| | | | SARS-CoV1 | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-103 | Ab | SARS-CoV2 | 229E, HKU1, | | | S; Unk | B-cells; SARS-CoV1 | 779 | QVQLVESGGGSVQAGGSLRLSCAASGSI |
| | | (weak) | NL63, OC43, | | | | Human Patient | | SSITYLGWFRQAPGKEREGVAALITDSG |
| | | | SARS-CoV1 | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-104 | Ab | SARS-CoV2 | 229E, HKU1, | | | S; Unk | B-cells; SARS-CoV1 | 780 | QVQLVESGGGSVQAGGSLRLSCAASGSI |
| | | (weak) | NL63, OC43, | | | | Human Patient | | SSITYLGWFRQAPGKEREGVAALITDSG |
| | | | SARS-CoV1 | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-105 | Ab | SARS-CoV2 | 229E, HKU1, | | | S; Unk | B-cells; SARS-CoV1 | 781 | QVQLVESGGGSVQAGGSLRLSCAASGSI |
| | | (weak) | NL63, OC43, | | | | Human Patient | | SSITYLGWFRQAPGKEREGVAALITDSG |
| | | | SARS-CoV1 | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-106 | Ab | SARS-CoV2 | 229E, HKU1, | | | S; Unk | B-cells; SARS-CoV1 | 782 | QVQLVESGGGSVQAGGSLRLSCAASGSI |
| | | (weak) | NL63, OC43, | | | | Human Patient | | SSITYLGWFRQAPGKEREGVAALITDSG |
| | | | SARS-CoV1 | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-107 | Ab | SARS-CoV1, | 229E, HKU1, | | | S; Unk | B-cells; SARS-CoV1 | 783 | QVQLVESGGGSVQAGGSLRLSCAASGSI |
| | | SARS-CoV2 | NL63, OC43 | | | | Human Patient | | SSITYLGWFRQAPGKEREGVAALITDSG |
| | | (weak) | | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-108 | Ab | SARS-CoV2 | 229E, HKU1, | | | S; Unk | B-cells; SARS-CoV1 | 784 | QVQLVESGGGSVQAGGSLRLSCAASGSI |
| | | (weak) | NL63, OC43, | | | | Human Patient | | SSITYLGWFRQAPGKEREGVAALITDSG |
| | | | SARS-CoV1 | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-109 | Ab | SARS-CoV1, | 229E, HKU1, | | | S; Unk | B-cells; SARS-CoV1 | 785 | QVQLVESGGGSVQAGGSLRLSCAASGSI |
| | | SARS-CoV2 | NL63, OC43 | | | | Human Patient | | SSITYLGWFRQAPGKEREGVAALITDSG |
| | | (weak) | | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-11 | Ab | SARS-CoV1, | 229E, HKU1, | | | S; Unk | B-cells; SARS-CoV1 | 786 | QVQLVESGGGSVQAGGSLRLSCAASGSI |
| | | SARS-CoV2 | NL63, OC43 | | | | Human Patient | | SSITYLGWFRQAPGKEREGVAALITDSG |
| | | | | | | | | | RTYYADSVKGRFTVSLDNAKNTVYLQM |
| | | | | | | | | | NSLKPEDTALYYCAAAEWGYEWPLYYAS |
| | | | | | | | | | SWYWGQGTQVTVSS |
| mAb-110 | Ab | SARS-CoV1, | 229E, HKU1, | | | S; Unk | B-cells; SARS-CoV1 | 787 | EVQLVESGGGVVQPGRSLRLSCAASGFT |
| | | SARS-CoV2 | NL63, OC43 | | | | Human Patient | | FSSYGMHWVRQAPGKGLEWVAVIWY |
| | | (weak) | | | | | | | DGSNKYYADSVKGRFTISRDNSKNTLYL |
| | | | | | | | | | QMNSLRAEDTAVYYCARVGSGRVYWG |
| | | | | | | | | | QGTLVTVSS |
| mAb-111 | Ab | 229E | HKU1, | | | S; Unk | B-cells; SARS-CoV1 | 788 | QVQLVESGPGLVRPSGTLSVTCAVSGDS |
| | | (weak), | NL63, OC43 | | | | Human Patient | | ISSDYWCTWVRQSPGKGLEWIGKISHS |
| | | SARS-CoV1, | | | | | | | GSLNYNPSLKSRVTMSVDKSKNHFSLKL |
| | | SARS-CoV2 | | | | | | | ASVTAADTAVYYCARVRIGASHHNFWS |
| | | (weak) | | | | | | | GYYTDAFDIWGQGTTVTVSS |
| mAb-112 | Ab | 229E | OC43 | | | S; Unk | B-cells; SARS-CoV1 | 789 | QVQLVQSGAEVKKPGESLKISCKASGYS |
| | | (weak), | | | | | Human Patient | | FTNYWVGWVRQMPAKGLEWMGIIWP |
| | | HKU1 | | | | | | | DDSDTRYRPSFQGQVTISVDKSISTAYLH |
| | | (weak), | | | | | | | WNSLKASDNGMYFCARAPLASCSGGRC |
| | | NL63, SARS- | | | | | | | PTYNRFDLWGQGTLVTVSS |
| | | CoV1, SARS- | | | | | | | |
| | | CoV2 | | | | | | | |
| | | (weak) | | | | | | | |
| mAb-113 | Ab | 229E, HKU1 | OC43 | | | S; Unk | B-cells; SARS-CoV1 | 790 | QVQLVQSGAEVKKPGSSMKVSCKASGV |
| | | (weak), | | | | | Human Patient | | NFRSYSFSWVRQAPGQGLEWMGGVIP |
| | | NL63, SARS- | | | | | | | YFPTANYADKFRDRVTITADESTGTVYLD |
| | | CoV1, SARS- | | | | | | | MSSLRSEDTAVYFCASEYFDGRSYHSFC |
| | | CoV2 | | | | | | | GLDVWGQGTTVTVSS |
| | | (weak) | | | | | | | |
| mAb-114 | Ab | 229E, HKU1, | OC43 | | | S; Unk | B-cells; SARS-CoV1 | 791 | QVQLVESGGGLVQPGGSLRLSCAASGF |
| | | NL63, SARS- | | | | | Human Patient | | TFSNYAMTWVRQAPGKGLKWVSGINP |
| | | CoV1, SARS- | | | | | | | SGDATFYTDSVKGQFTISRDNSKNILYLQ |
| | | CoV2 | | | | | | | MNRLRADDTAIYYCAKGLSFYGSGSDAF |
| | | (weak) | | | | | | | DVWGQGTTVTVSS |
| mAb-115 | Ab | 229E, HKU1 | OC43 | | | S; Unk | B-cells; SARS-CoV1 | 792 | QVQLVQSGAEVKKPGASVKVSCKASGY |
| | | (weak), | | | | | Human Patient | | TFTDYYVHWVRQVPGQGLEWMGWIS |
| | | NL63, SARS- | | | | | | | PDSGDTIRAQNFQGRVTMTRDTSMNT |
| | | CoV1, SARS- | | | | | | | AYMEVNRLRTDDTAIYYCARDLISVIRGL |
| | | CoV2 | | | | | | | GGGMDVWGQGTTVTVSS |
| | | (weak) | | | | | | | |
| mAb-116 | Ab | 229E | OC43 | | | S; Unk | B-cells; SARS-CoV1 | 793 | QVQLVQSGAEVKKPGASVKVSCKTSGY |
| | | (weak), | | | | | Human Patient | | TFTGHYIHWVRQAPGQGLEWMGWIN |
| | | HKU1 | | | | | | | PASGDTNYAQKFQGRVTKTRDTSITTAY |
| | | (weak), | | | | | | | MELNRLRSDDTAVYYCARGGPLPWSDL |
| | | NL63 | | | | | | | DIVGTFDYWGQGTLVTVSS |
| | | (weak), | | | | | | | |
| | | SARS-CoV1, | | | | | | | |
| | | SARS-CoV2 | | | | | | | |
| | | (weak) | | | | | | | |
| mAb-117 | Ab | 229E, HKU1, | OC43 | | | S; Unk | B-cells; SARS-CoV1 | 794 | EVQLVESGGGVVQPGKSLRLSCVPSGFS |
| | | NL63, SARS- | | | | | Human Patient | | FGTYGMHWVRQAPGKGPEWLAVMW |
| | | CoV1, SARS- | | | | | | | YDGITQYYADSVKGRFTISRDNSKETLYL |
| | | CoV2 | | | | | | | QMNSLTADDTGIYYCVKDQSSGDRLLYL |
| | | (weak) | | | | | | | GYFDLWGPGALVTVSS |
| mAb-118 | Ab | 229E, HKU1 | OC43 | | | S; Unk | B-cells; SARS-CoV1 | 795 | QVQLVQSGGGVVQPGKSLRLSCVASGF |
| | | (weak), | | | | | Human Patient | | SFGTYGMHWVRQAPGKGPEWLAVM |
| | | NL63 | | | | | | | WYDGITQYYADSVKGRFTISRDNSKETLY |
| | | (weak), | | | | | | | LQMNSLTADDTGVYYCVKDQSSGDRLL |
| | | SARS-CoV1, | | | | | | | YLGYFDLWGPGTLVTVSS |
| | | SARS-CoV2 | | | | | | | |
| | | (weak) | | | | | | | |
| mAb-119 | Ab | 229E, HKU1 | OC43 | | | S; Unk | B-cells; SARS-CoV1 | 796 | QVQLQESGPGLVKPSGTLSLTCAVSGAS |
| | | (weak), | | | | | Human Patient | | VSSDHWWSWVRQSPGKGLEWIGEVY |
| | | NL63 | | | | | | | HSGSTNYNPSLKSRVTISLDQSNNQFSLK |
| | | (weak), | | | | | | | LTSVTAADTAIYYCATMWGGLCTASNCY |
| | | SARS-CoV1, | | | | | | | GNPMDVWGQGTTVTVSS |
| | | SARS-CoV2 | | | | | | | |
| | | (weak) | | | | | | | |
| mAb-12 | Ab | SARS-CoV1, | 229E, HKU1, | | | S; Unk | B-cells; SARS-CoV1 | 797 | QVQLVQSGAEVRKPGSSVKLSCKASGG |
| | | SARS-CoV2 | NL63, OC43 | | | | Human Patient | | TFSTHAISWVRQAPGQRPEWMGGIMP |
| | | | | | | | | | IFGESKDTQKFQGRVTFTADESTTTAYM |
| | | | | | | | | | ELRSLRSDDTAIYYCVRDSDPYTATVTSN |
| | | | | | | | | | HYWYAMDVWGQGTTVTVSS |
| mAb-120 | Ab | 229E | OC43 | | | S; Unk | B-cells; SARS-CoV1 | 798 | QVQLQQWGAGVLKPSETLSLTCAVYGG |
| | | (weak), | | | | | Human Patient | | SFRGFFWSWIRQPPGKGLEWIGQITHS |
| | | HKU1 | | | | | | | GSTNYNSSLKSRLTISVDTSKNQFSLNLS |
| | | (weak), | | | | | | | SVTAADTAIYYCARGQGGYDLRRVGYGLT |
| | | NL63 | | | | | | | SWFDPWGQGILVTVSS |
| | | (weak), | | | | | | | |
| | | SARS-CoV1, | | | | | | | |
| | | SARS-CoV2 | | | | | | | |
| | | (weak) | | | | | | | |
| mAb-121 | Ab | 229E, HKU1 | OC43 | | | S; Unk | B-cells; SARS-CoV1 | 799 | QVQLVQSGAEVKKPGASVKVSCKASGY |
| | | (weak), | | | | | Human Patient | | TFTSYYIHWVRQAPGQGLEWLGVIHPS |
| | | NL63 | | | | | | | GGSTTFAQKFQGRVTMTRDTSTSTVYM |
| | | (weak), | | | | | | | ELGSLRSDDTAVYYCARVLAGSSHEWQL |
| | | SARS-CoV1, | | | | | | | THDAFDIWGQGTTVTVSS |
| | | SARS-CoV2 | | | | | | | |
| | | (weak) | | | | | | | |
| mAb-122 | Ab | 229E | OC43 | | | S; Unk | B-cells; SARS-CoV1 | 800 | EVQLVESGAEVKKPGASVILSCKASGYTV |
| | | (weak), | | | | | Human Patient | | TNYYIHWVRQAPGQGLEWMGWIDPD |
| | | HKU1 | | | | | | | SGVTNFAEKFQGRVTMTRDTSISTAYME |
| | | (weak), | | | | | | | LRWLESDDTAVYYCAKDLITVIRGLGGG |
| | | NL63 | | | | | | | MDVWGQGTTVTVSS |
| | | (weak), | | | | | | | |
| | | SARS-CoV1, | | | | | | | |
| | | SARS-CoV2 | | | | | | | |
| | | (weak) | | | | | | | |
| mAb-123 | Ab | 229E | NL63, OC43 | | | S; Unk | B-cells; SARS-CoV1 | 801 | EVQLLESGAEVKKPGSSVKVSCKASGGT |
| | | (weak), | | | | | Human Patient | | FSSDAISWVRQAPGQGLEWMGGIIPIF |
| | | HKU1 | | | | | | | GTTNYAQKFQGRVTITADESTNTAYMEL |
| | | (weak), | | | | | | | SSLRSEDTAVYYCARDGPYDSGGYHLNH |
| | | SARS-CoV1, | | | | | | | WGQGTLVTVSS |
| | | SARS-CoV2 | | | | | | | |
| | | (weak) | | | | | | | |
| mAb-124 | Ab | HKU1 | 229E, NL63, | | | S; Unk | B-cells; SARS-CoV1 | 802 | EVQLLESGGGVVNPGGSMRLSCAGSGF |
| | | (weak), | OC43 | | | | Human Patient | | TFSDHYMGWIRQAPGKGLEVISYISSSG |
| | | SARS-CoV1, | | | | | | | SFIRDADSVKGRFTISRDNAKNSVYLQM |
| | | SARS-CoV2 | | | | | | | NSLRAEDTAVYYCARMGPYGSGTFDYW |
| | | (weak) | | | | | | | GQGTLVTVSS |
| mAb-125 | Ab | 229E, HKU1 | OC43 | | | S; Unk | B-cells; SARS-CoV1 | 803 | EVQLVESGAEVRKPGSSVKVSCKATGGT |
| | | (weak), | | | | | Human Patient | | FSSYGITWVRQAPGQGLEWMGRIIPTL |
| | | NL63 | | | | | | | GRTNYAQKFQGRVTITADKSTSTAHMEL |
| | | (weak), | | | | | | | SSLRSEDTAVYYCARDLSTLQPDAIVNFD |
| | | SARS-CoV1, | | | | | | | YWGQGTLVTVSS |
| | | SARS-CoV2 | | | | | | | |
| | | (weak) | | | | | | | |
| mAb-126 | Ab | HKU1 | 229E, NL63, | | | S; Unk | B-cells; SARS-CoV1 | 804 | QVQLVQSGAEVKKPGSSVKVSCKTSGG |
| | | (weak), | OC43 | | | | Human Patient | | SFTSYVLSWVRQAPGQGLEWMGRIVP |
| | | SARS-CoV1, | | | | | | | NLGVANYAQKFQDRVTITADKSTTTAYL |
| | | SARS-CoV2 | | | | | | | ELRSLRSEDTAVYYCARDLYYDNGGYNY |
| | | (weak) | | | | | | | LDYWGPGTLVTVSS |
| mAb-127 | Ab | 229E | NL63, OC43 | | | S; Unk | B-cells; SARS-CoV1 | 805 | QVQLVESGGGLVQPGGSLRLSCAASGF |
| | | (weak), | | | | | Human Patient | | TFSNYNMVWVRQAPGKGLEWISYISSSS |
| | | HKU1 | | | | | | | STIYYADSVKGRFIISRDNAKNSLHLQMN |
| | | (weak), | | | | | | | SLRDEDTAVYYCVRDYCNSVSCYTYYYIG |
| | | SARS-CoV1, | | | | | | | MDVWGQGTTVTVSS |
| | | SARS-CoV2 | | | | | | | |
| | | (weak) | | | | | | | |
| mAb-128 | Ab | 229E | OC43 | | | S; Unk | B-cells; SARS-CoV1 | 806 | EVQLVQSGAEVKKPGASVKVSCKASGYS |
| | | (weak), | | | | | Human Patient | | STNYGFSWVRQAPGQGLEWMGWISV |
| | | HKU1, NL63 | | | | | | | HSGNANFAQKFQGRITMTTDTSTTTAY |
| | | (weak), | | | | | | | MELRNLRSDDTATYYCATSASSYSRYYFG |
| | | SARS-CoV1, | | | | | | | LDVWGQGTTVTVSS |
| | | SARS-CoV2 | | | | | | | |
| | | (weak) | | | | | | | |
| mAb-129 | Ab | HKU1, SARS- | 229E, NL63, | | | S; Unk | B-cells; SARS-CoV1 | 807 | EVQLLESGPGLVKPSQTLSLTCTVSNGFI |
| | | CoV1 | OC43 | | | | Human Patient | | SSGEYYWSWIRQSPGKGLEWIGYISHSG |
| | | (weak), | | | | | | | STYYNRSLKSRVTISLDTSRNQFSLNLSS |
| | | SARS-CoV2 | | | | | | | VTAADTAVYYCARDLAKWSYGYYYSGMD |
| | | (weak) | | | | | | | VWGQGTTVTVSS |
| mAb-13 | Ab | SARS-CoV1, | 229E, HKU1, | | | S; Unk | B-cells; SARS-CoV1 | 808 | QVQLVQSGAEVKKPGSSVKVSCKISGGT |
| | | SARS-CoV2 | NL63, OC43 | | | | Human Patient | | FKNSAFSWARQVPGQGFQWMGGIIP |
| | | | | | | | | | MFGVPHSVQMFQGRVTLTADESTSAVY |
| | | | | | | | | | MELSGLTSDDTAVYYCAREEYSGTVHNF |
| | | | | | | | | | FGMDVWGQGTTVTVSS |
| mAb-130 | Ab | HKU1 | 229E, OC43 | | | S; Unk | B-cells; SARS-CoV1 | 809 | EVQLVESGGGLVQPGRSLRLSCAASGFT |
| | | (weak), | | | | | Human Patient | | FDDYAMHWVRQVPGRGLEWVSGISW |
| | | NL63 | | | | | | | NSGTINYADSVMGRFTISRDNAKNSLYL |
| | | (weak), | | | | | | | QMNSLRAEDTALYYCAKDGRYCSGISCR |
| | | SARS-CoV1 | | | | | | | TGMDVWGQGTTVTVSS |
| | | (weak), | | | | | | | |
| | | SARS-CoV2 | | | | | | | |
| | | (weak) | | | | | | | |
| mAb-131 | Ab | 229E | NL63, OC43 | | | S; Unk | B-cells; SARS-CoV1 | 810 | EVQLVESGGGLVQPGESLRLSCAASGFN |
| | | (weak), | | | | | Human Patient | | FSPYGMNWVRQAPGKGLEWIAYIISGS |
| | | HKU1 | | | | | | | GTIYYADSVKGRFTISRDNAQSSLYLQM |
| | | (weak), | | | | | | | NSLRAEDTAVYYCARGLLDYLHDAFDIW |
| | | SARS-CoV1 | | | | | | | GQGTMVTVSS |
| | | (weak), | | | | | | | |
| | | SARS-CoV2 | | | | | | | |
| | | (weak) | | | | | | | |
| mAb-132 | Ab | 229E, HKU1 | OC43 | | | S; Unk | B-cells; SARS-CoV1 | 811 | EVQLQESGPGLVKPSETLSLTCAVSGGS |
| | | (weak), | | | | | Human Patient | | VSSDTDYWGWIRQPPGKGLEWIGSIHD |
| | | NL63 | | | | | | | SERTYYDPSLKSRVTISVDTSKNQFSLRL |
| | | (weak), | | | | | | | SSVTAADTALYFCASRHLDLLPIGSFDVW |
| | | SARS-CoV1 | | | | | | | GRGTMVTVSS |
| | | (weak), | | | | | | | |
| | | SARS-CoV2 | | | | | | | |
| | | (weak) | | | | | | | |
| mAb-133 | Ab | HKU1 | 229E, NL63, | | | S; Unk | B-cells; SARS-CoV1 | 812 | QVQLLESGGGLVQPGRSLRLSCTASGFR |
| | | (weak), | OC43 | | | | Human Patient | | FGDYTMTWFRQAPGKGLEWVGFIRSIA |
| | | SARS-CoV1 | | | | | | | YGGTTEHAASVEGRFIISRDDSKSIAYLQ |
| | | (weak), | | | | | | | MNSLKAEDTGVYFCTRGSGMFYGSSSG |
| | | SARS-CoV2 | | | | | | | MDVWGQGTTVTVSS |
| | | (weak) | | | | | | | |
| mAb-134 | Ab | 229E | NL63, OC43 | | | S; Unk | B-cells; SARS-CoV1 | 813 | EVQLLESGAEVKKPGSSVKVSCKTSGGT |
| | | (weak), | | | | | Human Patient | | FNSYTISWVRQAPGQGLEWMGGIIPIL |
| | | HKU1 | | | | | | | DTPHYAQKFRGRVTITADKSTSTAFMDL |
| | | (weak), | | | | | | | SSLTSEDTAVYYCAIRRDYSDYRDFDYW |
| | | SARS-CoV1 | | | | | | | GQGTLVTVSS |
| | | (weak), | | | | | | | |
| | | SARS-CoV2 | | | | | | | |
| | | (weak) | | | | | | | |
| mAb-135 | Ab | HKU1 | 229E, NL63, | | | S; Unk | B-cells; SARS-CoV1 | 814 | EVQLVESGPGLVKPSETLSLACTVSGGSI |
| | | (weak), | OC43 | | | | Human Patient | | SNYYWNWVRQPPGKGLEWIGNIYYSGS |
| | | SARS-CoV1 | | | | | | | TTFNPSLKSRVTISVDTSRNQFSLKLSSV |
| | | (weak), | | | | | | | TAADTAVYYCARQSSSWYNPYYFDQWG |
| | | SARS-CoV2 | | | | | | | QGTLVTVSS |
| | | (weak) | | | | | | | |
| mAb-136 | Ab | HKU1, SARS- | 229E, NL63, | | | S; Unk | B-cells; SARS-CoV1 | 815 | QVQLVQSGGGLVQPGESLRLSCAASGF |
| | | CoV1 | OC43 | | | | Human Patient | | NFSPYGMNWVRQAPGKGLEWIAYIISG |
| | | (weak), | | | | | | | SGTIYYADSVKGRFTISRDNAQSSLYLQM |
| | | SARS-CoV2 | | | | | | | NSLRAEDTAVYYCARGLLDYLHDAFDIW |
| | | (weak) | | | | | | | GQGTMVTVSS |
| mAb-137 | Ab | HKU1, SARS- | 229E, NL63, | | | S; Unk | B-cells; SARS-CoV1 | 816 | EVQLVESGAEVKNPGSSVKISCKSSGGTL |
| | | CoV1 | OC43 | | | | Human Patient | | GDYAISWVRQAPGLGLEWLGGIMPLH |
| | | (weak), | | | | | | | GTTGYSQRFRPRLTITADESARTAYMELT |
| | | SARS-CoV2 | | | | | | | ALSSEDSAIYYCARDPSILNTGNHHWYD |
| | | (weak) | | | | | | | LDLWGQGTEVTVSS |
| mAb-138 | Ab | HKU1 | 229E, NL63, | | | S; Unk | B-cells; SARS-CoV1 | 817 | EVQLLESGGGVVQPGRSLRLSCAASGFT |
| | | (weak), | OC43 | | | | Human Patient | | FNSYALFWVRQAPGKGLEWVAVVSYD |
| | | SARS-CoV1 | | | | | | | GNNKYYADSVKGRFTISRDNSKNTLYLQ |
| | | (weak), | | | | | | | MNSLKTEDTSVYYCARPRSGSYRQAIDY |
| | | SARS-CoV2 | | | | | | | WGLGTLVTVSS |
| | | (weak) | | | | | | | |
| mAb-139 | Ab | 229E, HKU1 | OC43 | | | S; Unk | B-cells; SARS-CoV1 | 818 | QVQLVQSGGGLVQPGGSLRLSCAASGFI |
| | | (weak), | | | | | Human Patient | | FSGHWMSWVRQAPGKGLEWVANIKQ |
| | | NL63, SARS- | | | | | | | DGREKHYVDSVKGRFTISRDNAKNSVSL |
| | | CoV1 | | | | | | | QMNSLRAEDTAVYYCVRQNVAIQYYYY |
| | | (weak), | | | | | | | AMDVWGQGTTVTVSS |
| | | SARS-CoV2 | | | | | | | |
| | | (weak) | | | | | | | |
| mAb-14 | Ab | SARS-CoV1, | 229E, HKU1, | | | S; Unk | B-cells; SARS-CoV1 | 819 | EVQLVESGGGVVQPGRSLRLSCAASGFT |
| | | SARS-CoV2 | NL63, OC43 | | | | Human Patient | | FSGYPMHWVRQAPGKGLEWVALISFD |
| | | | | | | | | | GDSKYYTDSVKARFAISRDNSKNTLFLQ |
| | | | | | | | | | MNSLRVADTALYYCARAKGGSYSNAFD |
| | | | | | | | | | YWGQGTLVTVSS |
| mAb-140 | Ab | 229E, HKU1 | OC43 | | | S; Unk | B-cells; SARS-CoV1 | 820 | QVQLVESGAEVKKPGSSVKVSCKASGGT |
| | | (weak), | | | | | Human Patient | | LSDYAISWVRQAPGQGLEWMGGIMPI |
| | | NL63, SARS- | | | | | | | FGSPGYAEIFQGRVTITADESKSTVYMEL |
| | | CoV1 | | | | | | | TSLRSEDTAVYYCARDPSILDTGNHHWY |
| | | (weak), | | | | | | | DLDIWGQGTMVTVSS |
| | | SARS-CoV2 | | | | | | | |
| | | (weak) | | | | | | | |
| mAb-141 | Ab | 229E, HKU1 | OC43 | | | S; Unk | B-cells; SARS-CoV1 | 821 | QVQLVQSGAEVKKPGSSVKVSCKTSGAT |
| | | (weak), | | | | | Human Patient | | YKNSAFSWARQAPGQGFQWMGGIIPL |
| | | NL63, SARS- | | | | | | | FGVPHYVQMFQGRVTITADESTSAVYM |
| | | CoV1 | | | | | | | ELSGLTSDDTAVYYCAREEYSGTVHNFF |
| | | (weak), | | | | | | | GMDVWGQGTTVTVSS |
| | | SARS-CoV2 | | | | | | | |
| | | (weak) | | | | | | | |
| mAb-142 | Ab | 229E, HKU1 | OC43 | | | S; Unk | B-cells; SARS-CoV1 | 822 | EVQLVESGGGLVQPGGSLRLSCSASEFT |
| | | (weak), | | | | | Human Patient | | LRSHAMHWVRQAPGKGLEYVSGISTDG |
| | | NL63, SARS- | | | | | | | SGRFYADSVKGRFTISRDNSKNKLFLQM |
| | | CoV1 | | | | | | | SSLRPEDTAVYYCVRDWGSSTHYDVFDL |
| | | (weak), | | | | | | | WGQGTMVTVSS |
| | | SARS-CoV2 | | | | | | | |
| | | (weak) | | | | | | | |
| mAb-143 | Ab | 229E, 229E | | | | S; Unk | B-cells; SARS-CoV1 | 823 | QVQLVQSGAEVKKPGSSVKVSCKASGG |
| | | (weak), | | | | | Human Patient | | TLADYAISWVRQAPGQGLEWMGGIKP |
| | | HKU1 | | | | | | | LHGAAGYSQHFRGRLSITADESASTAYM |
| | | (weak), | | | | | | | ELTGLRSEDTAMYYCARDPSILNTGNHH |
| | | NL63 | | | | | | | WYDLDLWGQGTMVTVSS |
| | | (weak), | | | | | | | |
| | | OC43 | | | | | | | |
| | | (weak), | | | | | | | |
| | | SARS-CoV1 | | | | | | | |
| | | (weak), | | | | | | | |
| | | SARS-CoV2 | | | | | | | |
| | | (weak) | | | | | | | |
| mAb-144 | Ab | 229E, HKU1, | OC43 | | | S; Unk | B-cells; SARS-CoV1 | 824 | QVQLVQSGAELKKPGSSVKVSCKASGG |
| | | NL63, SARS- | | | | | Human Patient | | TFYNSAFSWLRHAPGQGPEWMGGITP |
| | | CoV1 | | | | | | | SLGRVGYSERFLARLTITADESTSTVYME |
| | | (weak), | | | | | | | LTSLASEDTAVYYCARDASIVGTGNHLW |
| | | SARS-CoV2 | | | | | | | YGLDFWGHGTTVTVSS |
| | | (weak) | | | | | | | |
| mAb-145 | Ab | 229E, HKU1 | OC43 | | | S; Unk | B-cells; SARS-CoV1 | 825 | EVQLVESGGHVVLPGKSLRLSCAGSGFG |
| | | (weak), | | | | | Human Patient | | FPLYAMQWVRRAPGKGLEWVALVSYD |
| | | NL63, SARS- | | | | | | | SSNIRYADSVKGRFTISRDNSQNTLYLQ |
| | | CoV1 | | | | | | | MDSLRPEDTAMYYCARDNALQDGRPG |
| | | (weak), | | | | | | | YFDSWGQGTLVTVSS |
| | | SARS-CoV2 | | | | | | | |
| | | (weak) | | | | | | | |
| mAb-146 | Ab | 229E, HKU1 | OC43 | | | S; Unk | B-cells; SARS-CoV1 | 826 | EVQLVESGGGLVKPGGSLRLSCAASGFIF |
| | | (weak), | | | | | Human Patient | | SDYYMSWIRQAPGKGLEWVSYITGSGR |
| | | NL63, SARS- | | | | | | | TIHYADSVKGRFTISRDNAKNSVYLQMN |
| | | CoV1 | | | | | | | SLRAEDTAVYYCARGHRFLEFPLNYFDP |
| | | (weak), | | | | | | | WGQGTLVTVSS |
| | | SARS-CoV2 | | | | | | | |
| | | (weak) | | | | | | | |
| mAb-147 | Ab | 229E, HKU1 | OC43 | | | S; Unk | B-cells; SARS-CoV1 | 827 | EVQLLESGGHVVLPGKSLRLSCAGSGFG |
| | | (weak), | | | | | Human Patient | | FPLYAMQWVRRAPGKGLEWVALVSYD |
| | | NL63 | | | | | | | SSNIRYADSVKGRFTISRDNSQNTLYLQ |
| | | (weak), | | | | | | | MDSLRPEDTAMYYCARDNALQDGRPG |
| | | SARS-CoV1 | | | | | | | YFDSWGQGTLVTVSS |
| | | (weak), | | | | | | | |
| | | SARS-CoV2 | | | | | | | |
| | | (weak) | | | | | | | |
| mAb-148 | Ab | 229E, HKU1 | OC43 | | | S; Unk | B-cells; SARS-CoV1 | 828 | EVQLVESGGGVVQPGRSLRLSCAASGFT |
| | | (weak), | | | | | Human Patient | | FSKFAMHWVRQAPGKGLEWVAIISYDG |
| | | NL63 | | | | | | | SHKNYADSVKGRFTISRDNSKNTVYLQV |
| | | (weak), | | | | | | | DSLRAEDTAVYYCALLYGSGSYYNFVFFG |
| | | SARS-CoV1 | | | | | | | WKDGSDAWGPGTTVTVSS |
| | | (weak), | | | | | | | |
| | | SARS-CoV2 | | | | | | | |
| | | (weak) | | | | | | | |
| mAb-149 | Ab | 229E | OC43 | | | S; Unk | B-cells; SARS-CoV1 | 829 | QVQLVQSGAEVKKPGSSVKVSCKASGG |
| | | (weak), | | | | | Human Patient | | MFTDYAISWVRQAPGQRLEWMGGILP |
| | | HKU1, NL63 | | | | | | | AFAASGSPGYAPIFRGRATFSADVSTSTA |
| | | (weak), | | | | | | | YLELTNLKPEDTAVYYCARDPSILNTGNH |
| | | SARS-CoV1 | | | | | | | HWYDLDLWGQGTEVTVSS |
| | | (weak), | | | | | | | |
| | | SARS-CoV2 | | | | | | | |
| | | (weak) | | | | | | | |
| mAb-15 | Ab | SARS-CoV1, | 229E, HKU1, | | | S; Unk | B-cells; SARS-CoV1 | 830 | QVQLVQSGAELKKPGSSVRVSCKAAGG |
| | | SARS-CoV2 | NL63, OC43 | | | | Human Patient | | TLTNYAISWVRQAPGQGFEWMGGIMP |
| | | | | | | | | | VSHTAGYAQKFQGRVTFTADESATTAY |
| | | | | | | | | | MDLTSLRPEDTAIYYCARDPSIHYTGNH |
| | | | | | | | | | HWYDLDIWGQGTMVTVSS |
| mAb-150 | Ab | 229E | OC43 | | | S; Unk | B-cells; SARS-CoV1 | 831 | QVQLVQSGAELKKPGSSVRVSCKAAGG |
| | | (weak), | | | | | Human Patient | | TLTNYAISWVRQAPGQGFEWMGGIMP |
| | | HKU1 | | | | | | | VSHTAGYAQKFQGRVTFTADESATTAY |
| | | (weak), | | | | | | | MDLTSLRPEDTAIYYCARDPSIHYTGNH |
| | | NL63, SARS- | | | | | | | HWYDLDIWGQGTMVTVSS |
| | | CoV1 | | | | | | | |
| | | (weak), | | | | | | | |
| | | SARS-CoV2 | | | | | | | |
| | | (weak) | | | | | | | |
| mAb-151 | Ab | 229E | OC43 | | | S; Unk | B-cells; SARS-CoV1 | 832 | QVQLVQSGAEVKKPGSSMRVSCRVSG |
| | | (weak), | | | | | Human Patient | | GTFITHAMSWVRQAPGQGPEWMGGI |
| | | HKU1 | | | | | | | VPLFGRASYAQPSQTRVQITADESTSTVY |
| | | (weak), | | | | | | | LEVPSLTSEDTAVYYCVRDSEPYTATRSQ |
| | | NL63 | | | | | | | NHYWYDMDVWGQGTTVTVSS |
| | | (weak), | | | | | | | |
| | | SARS-CoV1 | | | | | | | |
| | | (weak), | | | | | | | |
| | | SARS-CoV2 | | | | | | | |
| | | (weak) | | | | | | | |
| mAb-152 | Ab | 229E | OC43 | | | S; Unk | B-cells; SARS-CoV1 | 833 | QVQLVQSGAELKKPGSSVRVSCKAAGG |
| | | (weak), | | | | | Human Patient | | TLTNYAISWVRQAPGQGFEWMGGIMP |
| | | HKU1 | | | | | | | VSHTAGYAQKFQGRVTFTADESATTAY |
| | | (weak), | | | | | | | MDLTSLRPEDTAIYYCARDPSIHYTGNH |
| | | NL63 | | | | | | | HWYDLDIWGQGTTVTVSS |
| | | (weak), | | | | | | | |
| | | SARS-CoV1 | | | | | | | |
| | | (weak), | | | | | | | |
| | | SARS-CoV2 | | | | | | | |
| | | (weak) | | | | | | | |
| mAb-153 | Ab | 229E | OC43 | | | S; Unk | B-cells; SARS-CoV1 | 834 | QVQLVQSGAEVKKPGSSVKVSCKASGG |
| | | (weak), | | | | | Human Patient | | TLSHYAISWVRQAPGQGLEWMGGIMP |
| | | HKU1 | | | | | | | VSGTVGYAQKFQGRVKFTADEYASTAY |
| | | (weak), | | | | | | | MELTNLRSEDSAVYFCARDPSIVDSGPH |
| | | NL63 | | | | | | | HWYDLDIWGQGTMVTVSS |
| | | (weak), | | | | | | | |
| | | SARS-CoV1 | | | | | | | |
| | | (weak), | | | | | | | |
| | | SARS-CoV2 | | | | | | | |
| | | (weak) | | | | | | | |
| mAb-154 | Ab | 229E | OC43 | | | S; Unk | B-cells; SARS-CoV1 | 835 | QVQLVESGAEVKRPGASVKVSCKASGY |
| | | (weak), | | | | | Human Patient | | AFSDYYMHWVRQAPGQGPEWMGWI |
| | | HKU1 | | | | | | | NPNSGDTGYPQKFRGWVTMTRDTSVS |
| | | (weak), | | | | | | | TAYMELKRLKSDDTAVYYCASGPNYFDY |
| | | NL63 | | | | | | | WGQGTLVTVSS |
| | | (weak), | | | | | | | |
| | | SARS-CoV1 | | | | | | | |
| | | (weak), | | | | | | | |
| | | SARS-CoV2 | | | | | | | |
| | | (weak) | | | | | | | |
| mAb-155 | Ab | 229E | OC43 | | | S; Unk | B-cells; SARS-CoV1 | 836 | EVQLLESGGGLVKPGGSLRLSCEASGFTF |
| | | (weak), | | | | | Human Patient | | SNYNMNWVRQAPGKGLEWVSSISGSS |
| | | HKU1 | | | | | | | SYISYADSVKGRFTISRDNAKNSLYLQMN |
| | | (weak), | | | | | | | SLRVEDTAVYYCARADRDYDFWSDPPLI |
| | | NL63 | | | | | | | DHWGQGTLVTVSS |
| | | (weak), | | | | | | | |
| | | SARS-CoV1 | | | | | | | |
| | | (weak), | | | | | | | |
| | | SARS-CoV2 | | | | | | | |
| | | (weak) | | | | | | | |
| mAb-156 | Ab | 229E | NL63, OC43 | | | S; Unk | B-cells; SARS-CoV1 | 837 | EVQLVESGGGLIQPGGSLRLSCAASGFT |
| | | (weak), | | | | | Human Patient | | VTDNYMSWVRQAPGKGLEWVSVLYSG |
| | | HKU1 | | | | | | | GSTYYADAVQGRFSISRDNSKNALYLQM |
| | | (weak), | | | | | | | NSLRAEDTAVYYCARGFGNGWSYYFDY |
| | | SARS-CoV1 | | | | | | | WGQGTLVTVSS |
| | | (weak), | | | | | | | |
| | | SARS-CoV2 | | | | | | | |
| | | (weak) | | | | | | | |
| mAb-157 | Ab | HKU1, SARS | 229E, NL63, | | | S; Unk | B-cells; SARS-CoV1 | 838 | EVQLVQSGGGLVQPGGSLRLSCAASGF |
| | | CoV1 | OC43 | | | | Human Patient | | TFSSYSMSWVRQAPGKGLEWVSYITRSS |
| | | (weak), | | | | | | | DNIYYAESVKGRFTISRDSAKNSLYLHMN |
| | | SARS-CoV2 | | | | | | | SLRDEDTAVYYCARDPGLEYSGNYFSYYY |
| | | (weak) | | | | | | | YAMDVWGQGTTVTVSS |
| mAb-158 | Ab | HKU1, SARS- | 229E, NL63, | | | S; Unk | B-cells; SARS-CoV1 | 839 | QVQLVQSGAEVKKPGSSVKVSCKASGG |
| | | CoV1 | OC43 | | | | Human Patient | | TFSTHAISWVRQAPGHGPEWMGGIIPL |
| | | (weak), | | | | | | | FGTSESAQRFQARVRFTADESTSTAYME |
| | | SARS-CoV2 | | | | | | | LSSLTSEDTAVYYCVRDSDPYTATSRNNH |
| | | (weak) | | | | | | | YWYDMDVWGQGTTVTVSS |
| mAb-159 | Ab | HKU1, SARS- | 229E, NL63, | | | S; Unk | B-cells; SARS-CoV1 | 840 | QVQLVQSGAEVKKPGSSVKVSCKVSGG |
| | | CoV1 | OC43 | | | | Human Patient | | RFSDYAISWLRQAPVKGLEWMGGIIPRL |
| | | (weak), | | | | | | | NRKGYSQDFQGRLTFTADESTSTAYMEL |
| | | SARS-CoV2 | | | | | | | SGLTSEDTAVYYCARDPTFLNSGNHFWY |
| | | (weak) | | | | | | | AVDIWGQGTTVTVSS |
| mAb-16 | Ab | SARS-CoV1, | 229E, HKU1, | | | S; Unk | B-cells; SARS-CoV1 | 841 | EVQLVESGAEVRTPGSSVKLSCKASGGT |
| | | SARS-CoV2 | NL63, OC43 | | | | Human Patient | | FSTHAFSWVRQAPGQRPEWMGGIIPIF |
| | | | | | | | | | GESKDTQKFQGRVTFTADESTTTVYMEL |
| | | | | | | | | | RSLKSDDTAIYYCVRDSDPYTATYRNNHY |
| | | | | | | | | | WYAMDVWGQGTTVTVSS |
| mAb-160 | Ab | HKU1, SARS- | 229E, NL63, | | | S; Unk | B-cells; SARS-CoV1 | 842 | EVQLVESGGGVVKPGGSLRVSCVGSGF |
| | | CoV1 | OC43 | | | | Human Patient | | TFSDHYMSWIRQAPGKGLEIISYISTDGS |
| | | (weak), | | | | | | | YINDADSVKGRFINSRDNAKNSVYLQLN |
| | | SARS-CoV2 | | | | | | | SLRAEDTAVYYCARMGPSGSGSLDYWG |
| | | (weak) | | | | | | | QGSLVTVSS |
| mAb-161 | Ab | HKU1, SARS- | 229E, NL63, | | | S; Unk | B-cells; SARS-CoV1 | 843 | EVQLVESGPTLVKPTQTLTLTCTFSGFSL |
| | | CoV1 | OC43 | | | | Human Patient | | NTRELGVGWIRQPPGKALEWLALIYWD |
| | | (weak), | | | | | | | DDKRYSPSLKSRLSITKDTSKNQVVLTLT |
| | | SARS-CoV2 | | | | | | | NMDPGDTATYYCAHTSELPPRRPYAAF |
| | | (weak) | | | | | | | DFWGQGTLVTVSS |
| mAb-162 | Ab | HKU1 | 229E, NL63, | | | S; Unk | B-cells; SARS-CoV1 | 844 | EVQLVESGGGVVQPGGSLRLSCVASGF |
| | | (weak), | OC43 | | | | Human Patient | | PFGRYAMHWVRQAPGQGLEWLTLISF |
| | | SARS-CoV1 | | | | | | | DSSNIEYSDSVQGRFTISRDNSRNTLFLQ |
| | | (weak), | | | | | | | MTSLRPEDTAVYFCARDLPPLDYWGQG |
| | | SARS-CoV2 | | | | | | | TLVTVSS |
| | | (weak) | | | | | | | |
| mAb-163 | Ab | HKU1 | 229E, OC43 | | | S; Unk | B-cells; SARS-CoV1 | 845 | QVQLQQWGAGLLKPSETLSLTCAVNGG |
| | | (weak), | | | | | Human Patient | | SFNNYYWSWIRQPPGKGPEWIGEVVHS |
| | | NL63 | | | | | | | GSTTYNPSLKSRVTISIDMSKNQFALKLN |
| | | (weak), | | | | | | | SVTAADTAVYYCARGFTFTYSDFLTGQRI |
| | | SARS-CoV1 | | | | | | | FEYWGQGTLVTVSS |
| | | (weak), | | | | | | | |
| | | SARS-CoV2 | | | | | | | |
| | | (weak) | | | | | | | |
| mAb-164 | Ab | HKU1 | 229E, OC43 | | | S; Unk | B-cells; SARS-CoV1 | 846 | EVQLVESGGGVVQPGRSLRLSCAASGFT |
| | | (weak), | | | | | Human Patient | | FSSYAMHWVRQAPGKGLEWVALISYD |
| | | NL63 | | | | | | | GDKKYYPDSVRGRFTISRDNSKNTLHLQ |
| | | (weak), | | | | | | | MNSLRLEDTAVYYCARSYGGSYSTVGY |
| | | SARS-CoV1 | | | | | | | WGQGALVTVSS |
| | | (weak), | | | | | | | |
| | | SARS-CoV2 | | | | | | | |
| | | (weak) | | | | | | | |
| mAb-165 | Ab | HKU1 | 229E, OC43 | | | S; Unk | B-cells; SARS-CoV1 | 847 | QVQLVQSGGDSVNPGGSLRLSCAGSGF |
| | | (weak), | | | | | Human Patient | | SVRDVWMSWVRQAPGKGLEWIGRIKS |
| | | NL63 | | | | | | | EADGGSSDYRASLKDRFSIWRDASKNTL |
| | | (weak), | | | | | | | YLQVNGLQTEDTAIYFCSWNDVGWAFT |
| | | SARS-CoV1 | | | | | | | FWGQGTLVTVSS |
| | | (weak), | | | | | | | |
| | | SARS-CoV2 | | | | | | | |
| | | (weak) | | | | | | | |
| mAb-166 | Ab | HKU1 | 229E, NL63, | | | S; Unk | B-cells; SARS-CoV1 | 848 | QVQLVQSGAEVKKAGSSVKVSCKASGG |
| | | (weak), | OC43 | | | | Human Patient | | PFSSFAISWVRQAPGQGLEWLGGIMTV |
| | | SARS-CoV1 | | | | | | | FGPAHYAQKSRDRISITADESTSTSYLEL |
| | | (weak), | | | | | | | SSLTSDDTAVYYCAAEERSGTNHNYYGLD |
| | | SARS-CoV2 | | | | | | | VWGQGTTVTVSS |
| | | (weak) | | | | | | | |
| mAb-167 | Ab | HKU1 | 229E, NL63, | | | S; Unk | B-cells; SARS-CoV1 | 849 | EVQLVESGGGLIQPGGSLRLSCAASGFP |
| | | (weak), | OC43 | | | | Human Patient | | FSGTYMTWVRQAPGKGLEWVSIIYSGG |
| | | SARS-CoV1 | | | | | | | DTYYADSVKGRFTISRDNSKNTLFLQMN |
| | | (weak), | | | | | | | SLRVEDTAMYYCARDREMAIITERSYGL |
| | | SARS-CoV2 | | | | | | | DVWGQGTMVTVSS |
| | | (weak) | | | | | | | |
| mAb-168 | Ab | HKU1 | 229E, NL63, | | | S; Unk | B-cells; SARS-CoV1 | 850 | EVQLVESGGGLVQPGGSLRLSCGASGFT |
| | | (weak), | OC43 | | | | Human Patient | | FSDYAMSWVRQAPGKGLEWVSTISGSG |
| | | SARS-CoV1 | | | | | | | DKTYYADSLKGRFTNSRDNSKSTLYLQM |
| | | (weak), | | | | | | | TSLRAEDTAVYFCAKDRYCSGGSCFYDAF |
| | | SARS-CoV2 | | | | | | | DIWGQGTTVTVSS |
| | | (weak) | | | | | | | |
| mAb-169 | Ab | HKU1 | 229E, NL63, | | | S; Unk | B-cells; SARS-CoV1 | 851 | EVQLVESGGGVVQPGRSLRLSCEASGFT |
| | | (weak), | OC43 | | | | Human Patient | | FADYPMHWVRQAPGKGLEWVAVISSH |
| | | SARS-CoV1 | | | | | | | GRSQGYAASVKGRFTFSRDNSQSSLFLQ |
| | | (weak), | | | | | | | LNSLRVEDTAVYFCAREAQSSGRAGCLD |
| | | SARS-CoV2 | | | | | | | AWGQGTLVTVSS |
| | | (weak) | | | | | | | |
| mAb-17 | Ab | SARS-CoV1, | 229E, HKU1, | | | S; Unk | B-cells; SARS-CoV1 | 852 | QVQLVQSGAELKKPGSSVRVSCKAAGG |
| | | SARS-CoV2 | NL63, OC43 | | | | Human Patient | | TLTNYAISWVRQAPGQGFEWMGGIMP |
| | | | | | | | | | VSHTAGYAQKFQGRVTFTADESATTAY |
| | | | | | | | | | MDLTSLRPEDTAIYYCARDPSIHYTGNH |
| | | | | | | | | | HWYDLDIWGQGTMVTVSS |
| mAb-170 | Ab | HKU1 | 229E, NL63, | | | S; Unk | B-cells; SARS-CoV1 | 853 | QVQLVQSGAEVREPGSSVKLSCKTSGGT |
| | | (weak), | OC43 | | | | Human Patient | | FSTHAISWVRQAPGQRPEWMGGIMPI |
| | | SARS-CoV1 | | | | | | | FGESKDTQKFQGRVTFTADESTTTAYME |
| | | (weak), | | | | | | | LRSLKSDDTAIYYCVRDSDPYTATVRSNH |
| | | SARS-CoV2 | | | | | | | YWYAMDVWGQGTTVTVSS |
| | | (weak) | | | | | | | |
| mAb-171 | Ab | HKU1 | 229E, NL63, | | | S; Unk | B-cells; SARS-CoV1 | 854 | EVQLVQSGAEVKKPGSSVKVSCKVSGG |
| | | (weak), | OC43 | | | | Human Patient | | RFSDYAISWLRQAPVKGLEWMGGIIPRL |
| | | SARS-CoV1 | | | | | | | NRKGYSQDFQGRLTFTADESTSTAYMEL |
| | | (weak), | | | | | | | SGLTSEDTAVYYCARDPTFLNSGNHFWY |
| | | SARS-CoV2 | | | | | | | AVDIWGQGTTVTVSS |
| | | (weak) | | | | | | | |
| mAb-172 | Ab | HKU1 | 229E, NL63, | | | S; Unk | B-cells; SARS-CoV1 | 855 | QVQLQESGRGLVKPSETLSLTCTVSGGSI |
| | | (weak), | OC43 | | | | Human Patient | | TSSRYYWGWIRQPPGRNLEWIGSIHYS |
| | | SARS-CoV1 | | | | | | | GTTSYNPSLWSRVAISVDTAQNQFSLRL |
| | | (weak), | | | | | | | NSVTAADTAVYYCAAPAPSNHESWSGT |
| | | SARS-CoV2 | | | | | | | DWFDPWGQGILVTVSS |
| | | (weak) | | | | | | | |
| mAb-173 | Ab | HKU1 | 229E, NL63, | | | S; Unk | B-cells; SARS-CoV1 | 856 | QVQLVQSGAEVKGPGASVKVSCKVSGY |
| | | (weak), | OC43 | | | | Human Patient | | SFITYDITWVRQAPGQGLEWMGWISTK |
| | | SARS-CoV1 | | | | | | | SGDTRYAQNVQGRVTMTTDTSTNTAY |
| | | (weak), | | | | | | | MELRNLKSDDTALYYCARTTPRGWEQ |
| | | SARS-CoV2 | | | | | | | WPVLEYWGQGTLVTVSS |
| | | (weak) | | | | | | | |
| mAb-174 | Ab | HKU1 | 229E, NL63, | | | S; Unk | B-cells; SARS-CoV1 | 857 | EVQLVESGGGLVQPGGSLRLSCEASGFN |
| | | (weak), | OC43 | | | | Human Patient | | FNSYSMSWVRQAPGKGLEWLSYISSRSS |
| | | SARS-CoV1 | | | | | | | TIKYASSVQGRFTVSRDNAKKSVYLQMN |
| | | (weak), | | | | | | | SLRDEDTAVYFCARELDSETYYNYNSLDV |
| | | SARS-CoV2 | | | | | | | WGQGTTVTVSS |
| | | (weak) | | | | | | | |
| mAb-175 | Ab | HKU1 | 229E, NL63, | | | S; Unk | B-cells; SARS-CoV1 | 858 | QVQLVESGGEVKKPGASVKVSCKASGY |
| | | (weak), | OC43 | | | | Human Patient | | TLSSYPISWVRQAPGHGLEWMGWINT |
| | | SARS-CoV1 | | | | | | | YNGRTNYEQMLQGRVTMTTDTSTSTAY |
| | | (weak), | | | | | | | MELRSLRSDDTAVYYCARVVFRHGQYD |
| | | SARS-CoV2 | | | | | | | DSSGRLAFDIWGQGTMVTVSS |
| | | (weak) | | | | | | | |
| mAb-176 | Ab | HKU1 | 229E, NL63, | | | S; Unk | B-cells; SARS-CoV1 | 859 | QVQLVQSGAELKKPGSSVRVSCKAAGG |
| | | (weak), | OC43 | | | | Human Patient | | TLTNYAISWVRQAPGQGFEWMGGIMP |
| | | SARS-CoV1 | | | | | | | VSHTAGYAQKFQGRVTFTADESATTAY |
| | | (weak), | | | | | | | MDLTSLRPEDTAIYYCARDPSIHYTGNH |
| | | SARS-CoV2 | | | | | | | HWYDLDIWGQGTMVTVSS |
| | | (weak) | | | | | | | |
| mAb-177 | Ab | HKU1 | 229E, NL63, | | | S; Unk | B-cells; SARS-CoV1 | 860 | EVQLLESGAEVKKPGSSVKVSCKTSGGT |
| | | (weak), | OC43 | | | | Human Patient | | FSTHAISWVRQAPGQGPEWMGGIIPLF |
| | | SARS-CoV1 | | | | | | | GTSEHAQRFQARVKFTADESTSTAYMEL |
| | | (weak), | | | | | | | SSLTPEDTAVYYCVRDSDPYTATSRNNH |
| | | SARS-CoV2 | | | | | | | YWYGMDVWGQGTTVTVSS |
| | | (weak) | | | | | | | |
| mAb-178 | Ab | SARS-CoV1 | 229E, HKU1, | | | S; Unk | B-cells; SARS-CoV1 | 861 | EVQLLESGGGVVQPGTSLRLSCAVSGF |
| | | (weak), | NL63, OC43 | | | | Human Patient | | MFKNYAIHWVRQAPGKGLEWVAVISF |
| | | SARS-CoV2 | | | | | | | DGSDISYTESVQGRFTISRDNSENMLYL |
| | | (weak) | | | | | | | QMNSLRAEDTAMYYCAREPDGIGAAGI |
| | | | | | | | | | SGYWGQGTLVTVSS |
| mAb-179 | Ab | SARS-CoV1 | 229E, HKU1, | | | S; Unk | B-cells; SARS-CoV1 | 862 | EVQLVESGGGLVKPGGSLRLSCVASGFT |
| | | (weak), | NL63, OC43 | | | | Human Patient | | FSDFYMSWIRQAPGKGLEWVSYISGSG |
| | | SARS-CoV2 | | | | | | | DTIYYADSVKGRFTVSRDNAKNSLFLQM |
| | | (weak) | | | | | | | SSLGAEDTAMYYCAREMATSFGYYFVLD |
| | | | | | | | | | VWGQGTTVTVSS |
| mAb-18 | Ab | SARS-CoV1, | 229E, HKU1, | | | S; Unk | B-cells; SARS-CoV1 | 863 | QVQLQQWGAGLLKPSETLSLSCAVYGG |
| | | SARS-CoV2 | NL63, OC43 | | | | Human Patient | | SFSGFYWSWIRQPPGKGLEWIGEINHS |
| | | | | | | | | | GSANYNPSLMSRVTISMDTSKKQFSLQL |
| | | | | | | | | | RSVTAADTAVYYCARGQESPIVGVTGR |
| | | | | | | | | | WFDPWGQGTLVTVSS |
| mAb-180 | Ab | SARS-CoV1 | 229E, HKU1, | | | S; Unk | B-cells; SARS-CoV1 | 864 | EVQLVESGGGVVQPGRSLRLSCAASEFT |
| | | (weak), | NL63, OC43 | | | | Human Patient | | FSTYAMHWVRQAPGKGLEWVALISYD |
| | | SARS-CoV2 | | | | | | | GINKYYADSVKGRFAISRDNSKNTLYLQV |
| | | (weak) | | | | | | | NSLRADDTAVYYCVRPYSGSYTNWFDL |
| | | | | | | | | | WGQGTLVTVSS |
| mAb-181 | Ab | SARS-CoV1 | 229E, HKU1, | | | S; Unk | B-cells; SARS-CoV1 | 865 | QVQLVQSGAEVKKPGSSVKVSCKASGG |
| | | (weak), | NL63, OC43 | | | | Human Patient | | MLSDYAISWVRQAPGQRLEWMGGIM |
| | | SARS-CoV2 | | | | | | | PAFGSPGYAQIFRGRATISADVSTSTAYL |
| | | (weak) | | | | | | | ELTSLKPEDTAVYYCARDPSILNTGNHH |
| | | | | | | | | | WYDLDIWGQGTMVTVSS |
| mAb-182 | Ab | 229E | NL63, OC43, | | | S; Unk | B-cells; SARS-CoV1 | 866 | QVQLVQSGAEVKMPGSSVKVSCKVSG |
| | | (weak), | SARS-CoV1 | | | | Human Patient | | GRFSDYAISWLRQAPLEGLEWMGGIVP |
| | | HKU1 | | | | | | | HLNRKGYSQKFQDRLTFTADDSTSTAYM |
| | | (weak), | | | | | | | ELSGLTSEDTAVYYCARDPTFLNTGNHF |
| | | SARS-CoV2 | | | | | | | WYAVDIWGQGTTVTVSS |
| | | (weak) | | | | | | | |
| mAb-183 | Ab | HKU1, SARS- | 229E, NL63, | | | S; Unk | B-cells; SARS-CoV1 | 867 | QVQLVQSGAEVKKPGSSVKVSCKASGG |
| | | CoV2 | OC43, SARS- | | | | Human Patient | | TFSTHAISWVRQAPGHGPEWLGGILPLF |
| | | (weak) | CoV1 | | | | | | GTSESAQRFQARVKITADESTSTAYMELS |
| | | | | | | | | | SLTSEDTAVYYCVRDSDPYTATSRNNHY |
| | | | | | | | | | WYAMDVWGQGTTVTVSS |
| mAb-184 | Ab | HKU1 | 229E, NL63, | | | S; Unk | B-cells; SARS-CoV1 | 868 | QVQLVQSGAEVKKPGSSVKVSCKVSGG |
| | | (weak), | OC43, SARS- | | | | Human Patient | | TFSNYAISWLRQAPGQGPEWMGGIIPA |
| | | SARS-CoV2 | CoV1 | | | | | | LSRVGYVRKFQARLTISADELTTTAYMDL |
| | | (weak) | | | | | | | SSLTSEDTAVYYCARDPSFLNTGNHFWY |
| | | | | | | | | | DFDLWGQGTTVTVSS |
| mAb-185 | Ab | SARS-CoV2 | 229E, HKU1, | | | S; Unk | B-cells; SARS-CoV1 | 869 | QVQLQQWGAGLLKPSETLSLTCAVYGG |
| | | (weak) | NL63, OC43, | | | | Human Patient | | SFSGFYWTWIRQPPGKGLEWIGEINHS |
| | | | SARS-CoV1 | | | | | | GSSNYDLSLKSRVTMSVDTSKNQFSLKLS |
| | | | | | | | | | SVTAADTAVYYCARGMISPRIPRTTRQR |
| | | | | | | | | | WFDTWGQGTLVTVSS |
| mAb-186 | Ab | SARS-CoV2 | 229E, HKU1, | | | S; Unk | B-cells; SARS-CoV1 | 870 | QVQLQQWGAGLLKPSETLSLTCGVYGG |
| | | (weak) | NL63, OC43, | | | | Human Patient | | SFSGYFWSWIRQSPGKGLEWIGEINHSR |
| | | | SARS-CoV1 | | | | | | SMSYNPSLKSRITMSVDTSKNQFSLNLN |
| | | | | | | | | | SVTAADTAVYFCARGKAHRNDFWSGYY |
| | | | | | | | | | PHWFDPWGQGILVTVSS |
| mAb-187 | Ab | SARS-CoV2 | 229E, HKU1, | | | S; Unk | B-cells; SARS-CoV1 | 871 | QVQLVQSGAEVKKPGSSVKISCKISGDTF |
| | | (weak) | NL63, OC43, | | | | Human Patient | | STNAISWLRQAPGREPEWMGGIVPLVG |
| | | | SARS-CoV1 | | | | | | PASYAQRPQGRLTITADEFTNTAYLELNS |
| | | | | | | | | | LRSEDTATYYCARDSDPYTATRRHNHYW |
| | | | | | | | | | YAMDVWGQGTTVTVSS |
| mAb-188 | Ab | SARS-CoV2 | 229E, HKU1, | | | S; Unk | B-cells; SARS-CoV1 | 872 | EVQLLESGPGLVKPSETLSLTCTVSGGSI |
| | | (weak) | NL63, OC43, | | | | Human Patient | | NSQYWNWIRQSPGKGLEWIGYVYYSGS |
| | | | SARS-CoV1 | | | | | | TNYNPSLKSRVTMSVDTSKNHFSLNLRS |
| | | | | | | | | | VTAADTAVYYCARGLVVRYFDGFPSGPII |
| | | | | | | | | | GAFDIWNQGTTVTVSS |
| mAb-189 | Ab | SARS-CoV2 | 229E, HKU1, | | | S; Unk | B-cells; SARS-CoV1 | 873 | QVQLVQSGAEVKKPGSSVKVSCKVSGG |
| | | (weak) | NL63, OC43, | | | | Human Patient | | TFSSHAISWVRQAPGQRPEWMGGIMP |
| | | | SARS-CoV1 | | | | | | IFGESKDTQKFQGRVTFTVDESTNTAYM |
| | | | | | | | | | ELTSLKSEDTAIYYCVRDPDPYTATVRHN |
| | | | | | | | | | HYWHGMDVWGQGTTVTVSS |
| mAb-19 | Ab | OC43, SARS- | 229E, HKU1, | | | S; Unk | B-cells; SARS-CoV1 | 874 | QVQLQQWGAGLLKPSETLSLTCAVYGG |
| | | CoV1, SARS- | NL63 | | | | Human Patient | | SFSGFHWSWIRQPPGKGLEWIGEVNHS |
| | | CoV2 | | | | | | | GSTKYNPSLKSRVTVSVDTSKNQFSLRLS |
| | | | | | | | | | SVTAADTAIYYCARGSLSREYDFLTAPQN |
| | | | | | | | | | GPWFDSWGQGALVTVSS |
| mAb-190 | Ab | SARS-CoV2 | 229E, HKU1, | | | S; Unk | B-cells; SARS-CoV1 | 875 | EVQLLESGPGLVKPSETLSLTCTVSGGSI |
| | | (weak) | NL63, OC43, | | | | Human Patient | | SSNNLYWGWIRQPPGKGLEWIGSIYYSG |
| | | | SARS-CoV1 | | | | | | STYYNPSLKSRVIIPVDTSKDQFSLRLSS |
| | | | | | | | | | VTAADTAVYYCARHSQKDIVLIPAAQSPI |
| | | | | | | | | | FDYWGQGTLVTVSS |
| mAb-191 | Ab | SARS-CoV2 | 229E, HKU1, | | | S; Unk | B-cells; SARS-CoV1 | 876 | EVQLVESGGGLVQPGGSLRLSCSASGFT |
| | | (weak) | NL63, OC43, | | | | Human Patient | | FSRYAMHWVRQAPGKGLEYVSAINLNG |
| | | | SARS-CoV1 | | | | | | DSTYYTDSVRGRFTISRDNSKNTLFLQMS |
| | | | | | | | | | NVRPEDTAFYYCVKDGGYYDSSGPGHW |
| | | | | | | | | | GQGTLVTVSS |
| mAb-192 | Ab | SARS-CoV2 | 229E, HKU1, | | | S; Unk | B-cells; SARS-CoV1 | 877 | QVQLVQSGGGVVQPGGSLRLSCADSG |
| | | (weak) | NL63, OC43, | | | | Human Patient | | NAFIANPMHWVRQAPGKGLEWLALIST |
| | | | SARS-CoV1 | | | | | | DGNNRHYADSVKGRFTFSRDNSKNSLYL |
| | | | | | | | | | QMDSLRPEDTGVYYCARESRSSGRAGC |
| | | | | | | | | | FDSWGQGTLVTVSS |
| mAb-193 | Ab | SARS-CoV2 | 229E, HKU1, | | | S; Unk | B-cells; SARS-CoV1 | 878 | QVQLVESGGGVVQPGTSLRLSCAATGF |
| | | (weak) | NL63, OC43, | | | | Human Patient | | TFTTYPMHWVRQAPGKGLEWVAVISY |
| | | | SARS-CoV1 | | | | | | DGMNQYYADFLKGRFTISRDNSKNTLYL |
| | | | | | | | | | QMNSLRADDTAVYYCARAYGGNYQNH |
| | | | | | | | | | FDHWGQGTLVTVSS |
| mAb-194 | Ab | SARS-CoV2 | 229E, HKU1, | | | S; Unk | B-cells; SARS-CoV1 | 879 | EVQLVQWGAGLLKPSETLSLRCAVYGGS |
| | | (weak) | NL63, OC43, | | | | Human Patient | | FNGFYWTWIRQAPGQGLEWIAEINHSG |
| | | | SARS-CoV1 | | | | | | TTNYNPSLKSRVTISIDTSKKQFSLSLKS |
| | | | | | | | | | VTAADTAMYFCARGTISPIVGVPTPVVPR |
| | | | | | | | | | RGRSWFDPWGQGTLVTVSS |
| mAb-195 | Ab | SARS-CoV2 | 229E, HKU1, | | | S; Unk | B-cells; SARS-CoV1 | 880 | QVQLVQSGAEVKKPGSSVKVSCKASGG |
| | | (weak) | NL63, OC43, | | | | Human Patient | | TFSTHAISWVRQAPGHGPEWLGGIIPLF |
| | | | SARS-CoV1 | | | | | | GTSESAQRFQARVKITADESTSTAYMELS |
| | | | | | | | | | SLTSEDTAVYYCVRDSDPYTATSRNNHY |
| | | | | | | | | | WYAMDVWGQGTTVTVSS |
| mAb-196 | Ab | SARS-CoV2 | 229E, HKU1, | | | S; Unk | B-cells; SARS-CoV1 | 881 | QVQLVQSGAEVREPGSSVKLSCKTSGGP |
| | | (weak) | NL63, OC43, | | | | Human Patient | | FSTHAFSWVRQAPGQRPEWMGGIMP |
| | | | SARS-CoV1 | | | | | | VFGESKDTQKFKGRVTFTADASTTTTYM |
| | | | | | | | | | ELRSLKSDDTAIYYCVRDSDPYTATSSHN |
| | | | | | | | | | HYWYAMDVWGQGTTVTVSS |
| mAb-197 | Ab | SARS-CoV2 | 229E, HKU1, | | | S; Unk | B-cells; SARS-CoV1 | 882 | QVQLVESGGGLVKPGGSLRLSCAASGFT |
| | | (weak) | NL63, OC43, | | | | Human Patient | | FSDYYMIWIRQAPGKGLEWLSYISHTAS |
| | | | SARS-CoV1 | | | | | | TIYYADSVKGRFTISRDNAKNSLFLQMNS |
| | | | | | | | | | LTAEDTAVYYCARDRGSGVIDPWGQGT |
| | | | | | | | | | LVTVSS |
| mAb-2 | Ab | SARS-CoV1, | 229E, HKU1, | | | S; Unk | B-cells; SARS-CoV1 | 883 | QVQLVESGGGLVQPGGSLRLSCSASGFT |
| | | SARS-CoV2 | NL63, OC43 | | | | Human Patient | | FSRFAMHWVRQAPGKGLEYVSAINLNG |
| | | | | | | | | | DSTYYTDSVRGRFTISRDNSKNTLYLQMS |
| | | | | | | | | | SVRPDDTAFYYCVKDGGYYDSSGPGHW |
| | | | | | | | | | GQGTLVTVSS |
| mAb-20 | Ab | OC43, SARS- | 229E, HKU1, | | | S; Unk | B-cells; SARS-CoV1 | 884 | EVQLVESGPGLVKPSETLSLTCTVSGASV |
| | | CoV1, SARS- | NL63 | | | | Human Patient | | TAGSSYWGWIRQPPGKGLEWIGYMFSS |
| | | CoV2 | | | | | | | GNTKYNPSLKSRVTISADTSKNQFSLRLS |
| | | | | | | | | | SVTAADTAVYFCARVGWVRYFDWSKPY |
| | | | | | | | | | YYFDLWGRGTLVTVSS |
| mAb-21 | Ab | SARS-CoV1, | 229E, HKU1, | | | S; Unk | B-cells; SARS-CoV1 | 885 | EVQLVESGGGLVQPGGSLRLSCAASGFT |
| | | SARS-CoV2 | NL63, OC43 | | | | Human Patient | | FSTSAMSWVRQAPGKGLEWVSRIGGG |
| | | | | | | | | | GGRTKYADSVKGRFTISRDNSKNTLYLQ |
| | | | | | | | | | MNSLRADDTAVYYCAKCDLVRYFDWLG |
| | | | | | | | | | EENNWFDPWGQGTLVTVSS |
| mAb-22 | Ab | SARS-CoV1, | 229E, HKU1, | | | S; Unk | B-cells; SARS-CoV1 | 886 | QVQLVQSGAEVRKPGSSVKLSCKASGG |
| | | SARS-CoV2 | NL63, OC43 | | | | Human Patient | | TFSTHAISWVRQAPGQRPEWMGGIMP |
| | | | | | | | | | IFGESKDTQKFQGRVTFTADESTTTAYM |
| | | | | | | | | | ELRSLKSDDTAIYYCVRDSDPYTATVRNN |
| | | | | | | | | | HYWYALDVWGPGTMVTVSS |
| mAb-23 | Ab | SARS-CoV1, | 229E, HKU1, | | | S; Unk | B-cells; SARS-CoV1 | 887 | EVQLVESGGGLSQPGGSLRLSCAASGFT |
| | | SARS-CoV2 | NL63, OC43 | | | | Human Patient | | VRTYCMNWVRQAPGKGLEWVSLVCG |
| | | | | | | | | | DNIDYYPDSVKGRFSISRDDSKNTLLLH |
| | | | | | | | | | MDSLRVEDTAVYYCARATPPGGGTGW |
| | | | | | | | | | PYFDFWGQGTLVTVSS |
| mAb-24 | Ab | SARS-CoV1 | 229E, HKU1, | | | S; Unk | B-cells; SARS-CoV1 | 888 | QVQLVQSGAEMRKPGSSVKVSCKASGG |
| | | (weak), | NL63, OC43 | | | | Human Patient | | TFSRYCFSWVRQAPGQRLEWMGGIMS |
| | | SARS-CoV2 | | | | | | | ILGAHYAQKFOGRVTFTADESTNTAYME |
| | | | | | | | | | LISLTSEDTAVYYCAREEPSGTYHNYYGL |
| | | | | | | | | | DVWGQGTTVTVSS |
| mAb-25 | Ab | SARS-CoV1, | 229E, HKU1, | | | S; Unk | B-cells; SARS-CoV1 | 889 | QVQLQESGPGLVKPSGTLSLTCAVSGGS |
| | | SARS-CoV2 | NL63, OC43 | | | | Human Patient | | ISSSDWCSWVRQPPGKGPEWIAEISHS |
| | | | | | | | | | GSTNYNPSLKSRVTMSVDRSKNQFSLNL |
| | | | | | | | | | NSVTAADTAVYYCAARIRGATHYDFWS |
| | | | | | | | | | GFWAGPFDIWGQGTTVTVSS |
| mAb-26 | Ab | SARS-CoV1, | 229E, HKU1, | | | S; Unk | B-cells; SARS-CoV1 | 890 | EVQLQESGPGLVKPSGTLSLTCGVSGVSI |
| | | SARS-CoV2 | NL63, OC43 | | | | Human Patient | | SSSSWWSWVRQPPGRGLEWIGEISPSG |
| | | | | | | | | | STSYNPSFRSRLTMSVDKSRNQLSLKLSS |
| | | | | | | | | | VTAADTAVYYCARTQSNDFWSGYYTAA |
| | | | | | | | | | FDLWGQGTMVTVSS |
| mAb-27 | Ab | SARS-CoV1, | 229E, HKU1, | | | S; Unk | B-cells; SARS-CoV1 | 891 | QVQLVQWGAGLLKPSETLSLTCAVYGG |
| | | SARS-CoV2 | NL63, OC43 | | | | Human Patient | | SFSGFHWSWIRQPPGKGLEWIGEINHS |
| | | | | | | | | | GSTKYNPSLKSRVTISVDTSKNQFSLRLR |
| | | | | | | | | | SVTAADTAIYYCARGSLSREYDFLTAPQN |
| | | | | | | | | | GPWFDSWGQGALVTVSS |
| mAb-28 | Ab | SARS-CoV1, | 229E, HKU1, | | | S; Unk | B-cells; SARS-CoV1 | 892 | EVQLVESGPELKKPGSSVRVSCKASGGS |
| | | SARS-CoV2 | NL63, OC43 | | | | Human Patient | | FSNFAVSWVRQAPGQRLEWMGGVIPR |
| | | | | | | | | | FGRSEYVQKFQGRVTITVDEAASTAYME |
| | | | | | | | | | LSSLRSEDTAIYYCVLDTTSANPHNWYG |
| | | | | | | | | | MDVWGQGTTVTVSS |
| mAb-29 | Ab | SARS-CoV1, | 229E, HKU1, | | | S; Unk | B-cells; SARS-CoV1 | 893 | QVQLVQSGGGVVNPGGSLRLSCVGSGF |
| | | SARS-CoV2 | NL63, OC43 | | | | Human Patient | | TFSDYYMGWIRQAPGKGLEVISYISSTGS |
| | | | | | | | | | YIRDADSVKGRFTISRDNAENSVYLQMN |
| | | | | | | | | | SLRGEDTAVYYCARMGPYGSGSFDYWG |
| | | | | | | | | | LGTLVTVSS |
| mAb-3 | Ab | SARS-CoV1, | 229E, HKU1, | | | S; Unk | B-cells; SARS-CoV1 | 894 | QVQLQESGPGLVRPSETLSLTCTVSGDS |
| | | SARS-CoV2 | NL63, OC43 | | | | Human Patient | | VSSSDYHWGWIRQPPGKGLEWIGSIYY |
| | | | | | | | | | GGRSHFNPSLKSRVAIFVDTSNNQFSLRL |
| | | | | | | | | | NSVTASDTAVYFCAGRHQELLPMGSFD |
| | | | | | | | | | MWAQGTTVTVSS |
| mAb-30 | Ab | SARS-CoV2 | 229E, HKU1, | | | S; Unk | B-cells; SARS-CoV1 | 895 | PGPACTVWAEVKKPGSSVKVSCKASGG |
| | | | NL63, OC43, | | | | Human Patient | | MFSDYAISWVRQAPGQRLEWMGGIM |
| | | | SARS-CoV1 | | | | | | PGLGSPGYAQIFRGRATISADVSTSTAYL |
| | | | | | | | | | ELTSLKPEDTAVYYCARDPSILNTGNHH |
| | | | | | | | | | WYDLDIWGQGTQVTVSS |
| mAb-31 | Ab | SARS-CoV2 | 229E, HKU1, | | | S; Unk | B-cells; SARS-CoV1 | 896 | QVQLVQSGAEVKKPGSSVKVSCKASGG |
| | | | NL63, OC43, | | | | Human Patient | | TSSTHAISWVRQAPGQGLEWMGGIIPIF |
| | | | SARS-CoV1 | | | | | | GTTNYAQKFQDRVTITADESTSTAYMEL |
| | | | | | | | | | SSLRSEDTAVYFCVRDGAYDSSGYYSTQ |
| | | | | | | | | | WGQGTLVTVSS |
| mAb-32 | Ab | SARS-CoV1, | 229E, HKU1, | | | S; Unk | B-cells; SARS-CoV1 | 897 | EVQLVQSGAEVKKPGSSVKVSCKVSGG |
| | | SARS-CoV2 | NL63, OC43 | | | | Human Patient | | RFSDYAISWLRQAPVKGLEWMGGIIPRL |
| | | | | | | | | | NRKGYSQDFQGRLTFTADESTSTAYMEL |
| | | | | | | | | | SGLTSEDTAVYYCARDPTFLNSGNHFWY |
| | | | | | | | | | AVDIWGQGTTVTVSS |
| mAb-33 | Ab | SARS-CoV1, | 229E, HKU1, | | | S; Unk | B-cells; SARS-CoV1 | 898 | QVQLVQSGAEVKKPGSSVTVSCKVSGG |
| | | SARS-CoV2 | NL63, OC43 | | | | Human Patient | | RFSDYAISWLRQAPVEGLEWMGGIIPHL |
| | | | | | | | | | NKKGYSQKFQDRITFTADESTSTAYMEL |
| | | | | | | | | | SGLTSEDTAIYYCARDPTFLNTGNHFWY |
| | | | | | | | | | AVDIWGQGTTVTVSS |
| mAb-34 | Ab | SARS-CoV1, | 229E, HKU1, | | | S; Unk | B-cells; SARS-CoV1 | 899 | QVQLVQSGAEVKKPGASVKVSCKASGY |
| | | SARS-CoV2 | NL63, OC43 | | | | Human Patient | | TFSLFHVHWVRQAPGQGLEWMGWIN |
| | | | | | | | | | PHNGDTTFAERFQGRVALTRDTSINTAY |
| | | | | | | | | | MELSRLTSDDTAVYFCARDFGVRYDDSR |
| | | | | | | | | | QLMKYCDSWGQGTLVTVSS |
| mAb-35 | Ab | SARS-CoV1, | 229E, HKU1, | | | S; Unk | B-cells; SARS-CoV1 | 900 | QVQLVQSGAEVKKPGSSVKVSCKASGG |
| | | SARS-CoV2 | NL63, OC43 | | | | Human Patient | | TLADYAISWVRQAPGQGLEWMGGIKP |
| | | | | | | | | | LHGAAGYSQLFRGRLSITADESASTAYM |
| | | | | | | | | | ELTGLTSDDTAMYYCARDPSILNTGNHH |
| | | | | | | | | | WYDLDLWGQGTTVTVSS |
| mAb-36 | Ab | SARS-CoV1, | 229E, HKU1, | | | S; Unk | B-cells; SARS-CoV1 | 901 | QVQLVQSGPELKKPGSSVRVSCKASGGS |
| | | SARS-CoV2 | NL63, OC43 | | | | Human Patient | | ISSYAISWVRQAPGQRLEWMGGVLPM |
| | | | | | | | | | MGRESPVQKFKDRVTIAADESTSTAYM |
| | | | | | | | | | ELRSLSAEDTAVYYCVVDTTMADPHNW |
| | | | | | | | | | YGLDVWGQGTTVTVSS |
| mAb-37 | Ab | SARS-CoV1, | 229E, HKU1, | | | S; Unk | B-cells; SARS-CoV1 | 902 | QVQLVQSGPGLVRPSGTLSLTCAVSGDS |
| | | SARS-CoV2 | NL63, OC43 | | | | Human Patient | | ISGDYWCTWVRQTPGKGLEWIGKISHS |
| | | | | | | | | | GSINYNPSLKSRITMSVDKSKNQFSLKLN |
| | | | | | | | | | SVTAADTAMYYCARVRVGASHHNFWS |
| | | | | | | | | | GYYTDAFDIWGQGTTVTVSS |
| mAb-38 | Ab | SARS-CoV1, | 229E, HKU1, | | | S; Unk | B-cells; SARS-CoV1 | 903 | QVQLVQSGAEVKKPGSSVKVSCKVSGG |
| | | SARS-CoV2 | NL63, OC43 | | | | Human Patient | | TFSNYAISWLRQAPGQGPEWMGGIIPA |
| | | | | | | | | | LSRVGYARKFQARLTISADELTTTAYMDL |
| | | | | | | | | | SSLTSEDTAVYYCARDPSFLNTGNHFWY |
| | | | | | | | | | DFDMWGQGTTVTVSS |
| mAb-39 | Ab | SARS-CoV2 | 229E, HKU1, | | | S; Unk | B-cells; SARS-CoV1 | 904 | EVQLQQSGAEVKKPGSSVKVSCKASGG |
| | | | NL63, OC43, | | | | Human Patient | | TSSTHAISWVRQAPGQGLEWMGGIIPIF |
| | | | SARS-CoV1 | | | | | | GTTNYAQKFQDRVTITADESTSTAYMEL |
| | | | | | | | | | SSLRSEDTAVYFCVRDGAYDSSGYYSTQ |
| | | | | | | | | | WGQGTLVTVSS |
| mAb-4 | Ab | SARS-CoV1, | 229E, HKU1, | | | S; Unk | B-cells; SARS-CoV1 | 905 | EVQLVESGGGLVKPGGSLRLSCAASGFT |
| | | SARS-CoV2 | NL63, OC43 | | | | Human Patient | | FSDYYMNWVRQAPGKGLEWVSSISSSS |
| | | | | | | | | | YMYYADSMKGRFTISRDNAQNSLYLQM |
| | | | | | | | | | SSLRAEDTAVYYCARDFPGDTAVAGTGF |
| | | | | | | | | | NYWGQGTLVTVSS |
| mAb-40 | Ab | SARS-CoV2 | 229E, HKU1, | | | S; Unk | B-cells; SARS-CoV1 | 906 | QVQLVESGGDLVKPGRSLRLSCTASGFTI |
| | | | NL63, OC43, | | | | Human Patient | | GDYAMTWFRQAPGKGLECVAVIRSRAF |
| | | | SARS-CoV1 | | | | | | GGTTEYAASVKGRFIVSRDDSNSVAFLQ |
| | | | | | | | | | MNSLKTEDTAVYYCSRDLRRGYYDSNG |
| | | | | | | | | | HQQFDLWGQGTLVTVSS |
| mAb-41 | Ab | SARS-CoV2 | 229E, HKU1, | | | S; Unk | B-cells; SARS-CoV1 | 907 | QVQLVESGGGVVQPGRSLRLSCAASGF |
| | | | NL63, OC43, | | | | Human Patient | | TFSRYGMHWVRQAPGKGLEWVAVIYS |
| | | | SARS-CoV1 | | | | | | DGRNEYYADSVKGRFTISRDNSKNTLHL |
| | | | | | | | | | QMNSLGAADTAVYYCARDPGPITFFDW |
| | | | | | | | | | SPDKSRKSYYDYNGMDVWGQGTTVTV |
| | | | | | | | | | SS |
| mAb-42 | Ab | SARS-CoV2 | 229E, HKU1, | | | S; Unk | B-cells; SARS-CoV1 | 908 | EVQLLESGGGVVQPGTSLRLSCVASGFT |
| | | | NL63, OC43, | | | | Human Patient | | FSTFAMHWVRQAPGKGLEWVALISFDS |
| | | | SARS-CoV1 | | | | | | TNIRYANSVRGRFTISRDNSKNTLYLEVD |
| | | | | | | | | | SLRIEDTGVYYCARDLPPLDYWGQGTLV |
| | | | | | | | | | TVSS |
| mAb-43 | Ab | SARS-CoV1, | 229E, HKU1, | | | S; Unk | B-cells; SARS-CoV1 | 909 | QVQLVQSGAEVRKPGSSVKLSCKASGG |
| | | SARS-CoV2 | NL63, OC43 | | | | Human Patient | | TFSTHAISWVRQAPGQRPEWMGGIIPIF |
| | | | | | | | | | GESKDTQKFQGRVTFTADESTTTAYMEL |
| | | | | | | | | | RSLRSDDTAIYYCVRDSDPYTTTFSHNHY |
| | | | | | | | | | WYAMDVWGQGTTVTVSS |
| mAb-44 | Ab | SARS-CoV1, | 229E, HKU1, | | | S; Unk | B-cells; SARS-CoV1 | 910 | QVQLVQSGGGLVQPGGSLRVSCSASGF |
| | | SARS-CoV2 | NL63, OC43 | | | | Human Patient | | TFSSFAMHWVRQAPGKGLEYVAGISDN |
| | | | | | | | | | GHTTMYADSVKGRFTISRDNSKNTLYLQ |
| | | | | | | | | | LSSLRPEDTAVYFCVKDNVILPGAIVRPQ |
| | | | | | | | | | FDYWGQGTLVTVSS |
| mAb-45 | Ab | SARS-CoV2 | 229E, HKU1, | | | S; Unk | B-cells; SARS-CoV1 | 911 | QVQLVQSGAEVKKPGSSVKVSCKVSGG |
| | | | NL63, OC43, | | | | Human Patient | | MFSDYAISWVRQAPGQRLEWMGGIM |
| | | | SARS-CoV1 | | | | | | PGLGSPAYAQIFRGRVTISADISTSTAYL |
| | | | | | | | | | EVTSLRPEDTAVYYCARDPSILNTGNHHW |
| | | | | | | | | | YDLDMWGQGTTVTVSS |
| mAb-46 | Ab | SARS-CoV1, | 229E, HKU1, | | | S; Unk | B-cells; SARS-CoV1 | 912 | EVQLVESGGGVVQPGRSLRLSCVASGFT |
| | | SARS-CoV2 | NL63, OC43 | | | | Human Patient | | LAPYGMQWVRQAPGKGLEWVAFLSH |
| | | | | | | | | | DGSHLGYVDSVKGRFTISRDNSKNTLYLE |
| | | | | | | | | | MNSLRAEDTATYYCARDNVVQQNADN |
| | | | | | | | | | VGYFDFWGQGSLVTVSS |
| mAb-47 | Ab | SARS-CoV1, | 229E, HKU1, | | | S; Unk | B-cells; SARS-CoV1 | 913 | QVQLVQSGAEVKKPGSSVKVSCKASGG |
| | | SARS-CoV2 | NL63, OC43 | | | | Human Patient | | MFRDYAISWLRQAPGQRLEWMGGIM |
| | | | | | | | | | PAFGAPGYAQIFRGRATISADVSTTTAYL |
| | | | | | | | | | ELTSLMPDDTAVYYCARDPSILNTGNHH |
| | | | | | | | | | WYDLDLWGQGTTVTVSS |
| mAb-48 | Ab | SARS-CoV1, | 229E, HKU1, | | | S; Unk | B-cells; SARS-CoV1 | 914 | QVTLKESGAEVKKPGSSVKVSCKTSGGT |
| | | SARS-CoV2 | NL63, OC43 | | | | Human Patient | | FSTHAISWVRQAPGQGPEWMGGIIPLF |
| | | | | | | | | | GTSQHAQRFQARVKFTADESTNTAYME |
| | | | | | | | | | LSSLTPEDTAVYYCVRDSDPYTATSRNNH |
| | | | | | | | | | YWYGMDVWGHGTTVTVSS |
| mAb-49 | Ab | SARS-CoV1, | 229E, HKU1, | | | S; Unk | B-cells; SARS-CoV1 | 915 | QVQLVQSGAEVKKPGSSVKVSCKASGG |
| | | SARS-CoV2 | NL63, OC43 | | | | Human Patient | | MFSDYAISWVRQAPGQRLEWMGGIM |
| | | | | | | | | | PGLGSPAYAQSFGGRVTISADISTSTAYL |
| | | | | | | | | | EVTSLRPEDTAVYYCARDPSILNTGNHH |
| | | | | | | | | | WYDLDMWGQGTMVTVSS |
| mAb-5 | Ab | SARS-CoV1, | 229E, HKU1, | | | S; Unk | B-cells; SARS-CoV1 | 916 | EVQLVESGGGLVQPGRSLRLSCSASGFS |
| | | SARS-CoV2 | NL63, OC43 | | | | Human Patient | | FGDYAMSWFRQAPGKGLQWVGLIKTR |
| | | | | | | | | | AYGAATDYAASVQGRFIISRDDSKSIAYL |
| | | | | | | | | | QMNSLKTEDTAVYFCAREGTSLGYYYYY |
| | | | | | | | | | AMDVWGHGTTVTVSS |
| mAb-50 | Ab | SARS-CoV1, | 229E, HKU1, | | | S; Unk | B-cells; SARS-CoV1 | 917 | QVQLVQSGAEVKKPGSSVKVSCKASGG |
| | | SARS-CoV2 | NL63, OC43 | | | | Human Patient | | MFSDYAISWVRQAPGQRLEWMGGIM |
| | | | | | | | | | PGLGSPAYAQIFRGRVTISADISTSTAYL |
| | | | | | | | | | EVTSLRPEDTAVYYCARDPSILNTGNHHW |
| | | | | | | | | | YDLDMWGQGTTVTVSS |
| mAb-51 | Ab | SARS-CoV1, | 229E, HKU1, | | | S; Unk | B-cells; SARS-CoV1 | 918 | EVQLVESGGGLVQPGGSLRLSCLTSGFS |
| | | SARS-CoV2 | NL63, OC43 | | | | Human Patient | | FSSYWMIWVRQAPGKGLEWVANIEPD |
| | | | | | | | | | GNEEYYVDSVKGRFTISRDNAKNSLYLQ |
| | | | | | | | | | MNSLRAEDTAVYYCARGPIRHFGLDAFD |
| | | | | | | | | | IWGQGTTVTVSS |
| mAb-52 | Ab | SARS-CoV2 | 229E, HKU1, | | | S; Unk | B-cells; SARS-CoV1 | 919 | QVQLVQSGDEMKKPGSSVKVSCKASGD |
| | | | NL63, OC43, | | | | Human Patient | | TFSTHAISWVRQAPGQGPEWMGGIIPL |
| | | | SARS-CoV1 | | | | | | FGTASYAQTSQSRVKITADESTSTAYMEL |
| | | | | | | | | | SSLTSEDTAVYYCVRDSDPYTATSRNNHY |
| | | | | | | | | | WYGMDVWGQGTTVTVSS |
| mAb-53 | Ab | SARS-CoV1, | 229E, HKU1, | | | S; Unk | B-cells; SARS-CoV1 | 920 | QVQLVQSGAEVKKPGSSVKVSCKASGG |
| | | SARS-CoV2 | NL63, OC43 | | | | Human Patient | | MFSDYAISWVRQAPGQRLEWMGGIM |
| | | | | | | | | | PGLGSPAYAQSFGGRVTISADISTSTAYL |
| | | | | | | | | | EVTSLRPEDTAVYYCARDPSILNTGNHH |
| | | | | | | | | | WYDLDMWGQGTMVTVSS |
| mAb-54 | Ab | SARS-CoV1, | 229E, HKU1, | | | S; Unk | B-cells; SARS-CoV1 | 921 | EVQLVESGGGLVQPGGSLRLSCSASGFT |
| | | SARS-CoV2 | NL63, OC43 | | | | Human Patient | | FSNYAMYWVRQAPGKRPEYVSGISSNG |
| | | | | | | | | | GITYYADSVEGRFTVSRDNSKKSLYLQM |
| | | | | | | | | | SSLRPEDTAVYYCVKDLGATVTYDVFDV |
| | | | | | | | | | WGQGTMVTVSS |
| mAb-55 | Ab | SARS-CoV2 | 229E, HKU1, | | | S; Unk | B-cells; SARS-CoV1 | 922 | EVQLVESGGGLVQPGGSLRLSCAASGFT |
| | | | NL63, OC43, | | | | Human Patient | | FSSYSMSWVRQAPGKGLEWVSGHDGG |
| | | | SARS-CoV1 | | | | | | TTHYADSVKGRFTISRDDSMNTLSLQM |
| | | | | | | | | | NSLRAEDTAVYYCAKERDLPGRGGYFDH |
| | | | | | | | | | WGQGTLVTVSS |
| mAb-56 | Ab | SARS-CoV2 | 229E, HKU1, | | | S; Unk | B-cells; SARS-CoV1 | 923 | QVQLQQWGAGLLKPSETLSLTCAVYGG |
| | | | NL63, OC43, | | | | Human Patient | | SFRGFYWTWIRQPPGKGLEWIGEVSHS |
| | | | SARS-CoV1 | | | | | | GETNYNPSLKSRVTISVDTSKNQFSLNLI |
| | | | | | | | | | SVTAADTSVYYCARGYTAPIIREVPITFR |
| | | | | | | | | | PRWFDPWGQGTPVTVSS |
| mAb-57 | Ab | SARS-CoV1, | 229E, HKU1, | | | S; Unk | B-cells; SARS-CoV1 | 924 | QVQLVQSGGGVVNPGGSMRLSCAGSG |
| | | SARS-CoV2 | NL63, OC43 | | | | Human Patient | | FTFSDHYMGWIRQAPGKGLEVISYISSS |
| | | | | | | | | | GSFIRDADSVKGRFTISRDNAKNSVYLQ |
| | | | | | | | | | MNSLRAEDTAVYYCARMGPYGSGTFDY |
| | | | | | | | | | WGQGTLVTVSS |
| mAb-58 | Ab | SARS-CoV1, | 229E, HKU1, | | | S; Unk | B-cells; SARS-CoV1 | 925 | QVQLVQSGGNVVQPGGSLRLSCVGSEF |
| | | SARS-CoV2 | NL63, OC43 | | | | Human Patient | | SITFFAMQWVRRTPGKGLEWVALVSHD |
| | | | | | | | | | GSNIRYSDSVKGRFIISRDNAKNTLYLQL |
| | | | | | | | | | DSLTPEDTGIYYCARDHALQNGRPGYFD |
| | | | | | | | | | SWGQGSQVTVSS |
| mAb-59 | Ab | SARS-CoV1, | 229E, HKU1, | | | S; Unk | B-cells; SARS-CoV1 | 926 | QVQLVQSGGGLVQPGGSLRLSCSASGF |
| | | SARS-CoV2 | NL63, OC43 | | | | Human Patient | | TFNTYAMHWVRQAPGKRLEYVSSITRD |
| | | | | | | | | | GAGKFYADSVKGRFTISRDNSKNTLYQQ |
| | | | | | | | | | MSSLRPEDTAVYYCVREGQQWLGLYFD |
| | | | | | | | | | HWGQGALVTVSS |
| mAb-6 | Ab | SARS-CoV1, | 229E, HKU1, | | | S; Unk | B-cells; SARS-CoV1 | 927 | QVQLVQSGAEVKKPGASVKVSCRASGY |
| | | SARS-CoV2 | NL63, OC43 | | | | Human Patient | | TFSGYDINWVRQATGQGLEWMGWM |
| | | | | | | | | | NPNSGDTGYAHKFQGRVTMTRNSSIST |
| | | | | | | | | | AYMELSSLTSEDTAVYFCAREKKSFGPQY |
| | | | | | | | | | YYGSGEDWGQGTLVTVSS |
| mAb-60 | Ab | SARS-CoV1, | 229E, HKU1, | | | S; Unk | B-cells; SARS-CoV1 | 928 | EVQLVQSGAEMKKPGESLKISCKGSGYS |
| | | SARS-CoV2 | NL63, OC43 | | | | Human Patient | | FPNYWIGWVRQMPGKGLEWMAIMW |
| | | | | | | | | | PSDSDTRYSPSFQGQVTISADTSTSTVYL |
| | | | | | | | | | QWGSLKASDTAMYYCVRQRYCSGGSCF |
| | | | | | | | | | LFEDAFEIWGQGTMVTVSS |
| mAb-61 | Ab | SARS-CoV2 | 229E, HKU1, | | | S; Unk | B-cells; SARS-CoV1 | 929 | EVQLVESGGGLVQPGGSLRLSCAASGFI |
| | | | NL63, OC43, | | | | Human Patient | | FKNEPMNWIRQFPGKGLEWISNIRDNG |
| | | | SARS-CoV1 | | | | | | NDVYYADSVKGRFTVSRDNAKNSLYLQ |
| | | | | | | | | | MNSLRDDDTALYYCVRDTDWAFDSWG |
| | | | | | | | | | QGTLVTVSS |
| mAb-62 | Ab | SARS-CoV2 | 229E, HKU1, | | | S; Unk | B-cells; SARS-CoV1 | 930 | EVQLVESGPTLVKPTQTLTLTCTFSGFSLT |
| | | | NL63, OC43, | | | | Human Patient | | TRRQGVGWLRRPPGKALEWLALIYWD |
| | | | SARS-CoV1 | | | | | | DDKRYSPSLKSRLTITKDTSKNHVVLSLT |
| | | | | | | | | | NVGPADTATYYCAHTSELPPRRPYAAFD |
| | | | | | | | | | FWGQGTLVTVSS |
| mAb-63 | Ab | SARS-CoV2 | 229E, HKU1, | | | S; Unk | B-cells; SARS-CoV1 | 931 | EVQLLESGGGLVQPGGSLRLSCAASGFP |
| | | | NL63, OC43, | | | | Human Patient | | FSTDAMNWVRQAPGEGLEWVSTISDS |
| | | | SARS-CoV1 | | | | | | GRDTYYAASVRGRFTISRDNSKNTVYLQ |
| | | | | | | | | | MNSLRVEDTAVYYCANTNFLDYWGQG |
| | | | | | | | | | TLVTVSS |
| mAb-64 | Ab | SARS-CoV1, | 229E, HKU1, | | | S; Unk | B-cells; SARS-CoV1 | 932 | EVQLQESGPGLLKPSETLSLTCTVSGSPIA |
| | | SARS-CoV2 | NL63, OC43 | | | | Human Patient | | SNYWSWVRQPAGKGLEWIGRIDTSPTT |
| | | | | | | | | | DYNPSLKSRVIMSVDTSTSQFSLKMSSVT |
| | | | | | | | | | AADTAVYYCTRSFISFDSSGHPYYYYAMD |
| | | | | | | | | | VWGQGTTVTVSS |
| mAb-65 | Ab | SARS-CoV1, | 229E, HKU1, | | | S; Unk | B-cells; SARS-CoV1 | 933 | QVQLVQSGAEVRKPGSSVKLSCRASGG |
| | | SARS-CoV2 | NL63, OC43 | | | | Human Patient | | TFHTYTVNWVRQAPGQGLEWLGGIIPIF |
| | | | | | | | | | GTPTYAQRFQGKVSITADSSTNTVFMEL |
| | | | | | | | | | TSLTSEDTAVYYCTRETGTDEFDFWGQG |
| | | | | | | | | | ALVTVSS |
| mAb-66 | Ab | SARS-CoV2 | 229E, HKU1, | | | S; Unk | B-cells; SARS-CoV1 | 934 | EVQLVESGPGLVKPSGTLSLTCAVSGAS |
| | | | NL63, OC43, | | | | Human Patient | | VSSDHWWSWVRQSPGKGLEWIGEVY |
| | | | SARS-CoV1 | | | | | | HSGSTNYNPSLKSRVTISLDQSNNQFSLK |
| | | | | | | | | | LTSVTAADTAIYYCATMWGGLCTASNCY |
| | | | | | | | | | GNPMDVWGQGTTVTVSS |
| mAb-67 | Ab | SARS-CoV2 | 229E, HKU1, | | | S; Unk | B-cells; SARS-CoV1 | 935 | EVQLVESGPGLVKPSGTLSLTCAVTGAS |
| | | | NL63, OC43, | | | | Human Patient | | VSSDHWWSWVRQSPGKGLEWIGEVY |
| | | | SARS-CoV1 | | | | | | HSGSTNYNPSLKSRVTISLDQSNNQFSLK |
| | | | | | | | | | LTSVTAADTAIYYCATMWGGLCTASNCY |
| | | | | | | | | | GNPMDVWGQGTTVTVSS |
| mAb-68 | Ab | SARS-CoV1, | 229E, HKU1, | | | S; Unk | B-cells; SARS-CoV1 | 936 | QVQLQESGPGLVKPSGTLSLTCAVSGAS |
| | | SARS-CoV2 | NL63, OC43 | | | | Human Patient | | VSSDHWWSWVRQSPGKGLEWIGEVY |
| | | | | | | | | | HSGSTNYNPSLKSRVTISLDQSNNQFSLK |
| | | | | | | | | | LTSVTAADTAIYYCATMWGGLCTASNCY |
| | | | | | | | | | GNPMDVWGQGTTVTVSS |
| mAb-69 | Ab | SARS-CoV1, | 229E, HKU1, | | | S; Unk | B-cells; SARS-CoV1 | 937 | QVQLQESGGGLVQPGGSLRLSCEASGF |
| | | SARS-CoV2 | NL63, OC43 | | | | Human Patient | | SFSDFVMHWVRQVPGKGLEWVSRISH |
| | | | | | | | | | DGSITSYVDSVKGRFTVSRDNGKKTLYL |
| | | | | | | | | | QMNSPRTEDTAVYYCARDLAWTFFDY |
| | | | | | | | | | WGHGTLVTVSS |
| mAb-7 | Ab | SARS-CoV1, | 229E, HKU1, | | | S; Unk | B-cells; SARS-CoV1 | 938 | EVQLLESGGGLSRPGGSLRLSCAASGFIA |
| | | SARS-CoV2 | NL63, OC43 | | | | Human Patient | | SRNCMQWVRQAPGKGLEWVSIICGDEI |
| | | | | | | | | | TYIRDSVKGRFTISRDDSKNTLHLEMNSL |
| | | | | | | | | | RADDTAVYYCARATPPGGTTGWPYIDL |
| | | | | | | | | | WGQGTLVTVSS |
| mAb-70 | Ab | 229E, HKU1 | OC43 | | | S; Unk | B-cells; SARS-CoV1 | 939 | EVQLLESGGGLVQPGGSLRLSCAASGFP |
| | | (weak), | | | | | Human Patient | | FSTDAMNWVRQAPGEGLEWVSTISDT |
| | | NL63, SARS- | | | | | | | GRDTYYAASVKGRFTISRDNSKNTVYLQ |
| | | CoV1 | | | | | | | MNSLRAEDTAVYYCANTNFLDYWGQG |
| | | (weak), | | | | | | | TLVTVSS |
| | | SARS-CoV2 | | | | | | | |
| mAb-71 | Ab | SARS-CoV2 | 229E, HKU1, | | | S; Unk | B-cells; SARS-CoV1 | 940 | EVQLVESGGGLVQPGRSLRLSCAASGFIL |
| | | | NL63, OC43, | | | | Human Patient | | DDYAVHWVRLAPGKGLEWVSGITWNS |
| | | | SARS-CoV1 | | | | | | GYLGYADSVKGRFTISRDNAKNSLYLQM |
| | | | | | | | | | NSLRPEDTALYYCAKLGTDHPIGVDVW |
| | | | | | | | | | GQGTTVTVSS |
| mAb-72 | Ab | SARS-CoV2 | 229E, HKU1, | | | S; Unk | B-cells; SARS-CoV1 | 941 | QVQLVQSGGGLVQPGGSLRLSCSASGF |
| | | | NL63, OC43, | | | | Human Patient | | TFNTYTMHWVRQAPGKGLEYVSAISSN |
| | | | SARS-CoV1 | | | | | | GVVTYYADSVKGRFTISRDNSKNTLYLQ |
| | | | | | | | | | MSSLRAEDTAVYYCVKALYSSSWCPFDY |
| | | | | | | | | | WGQGALVTVSS |
| mAb-73 | Ab | SARS-CoV2 | 229E, HKU1, | | | S; Unk | B-cells; SARS-CoV1 | 942 | QVQLQQSGGGLVQPGGSLRLSCEASGF |
| | | | NL63, OC43, | | | | Human Patient | | NFNSYSMSWVRQAPGKGLEWLSYISSR |
| | | | SARS-CoV1 | | | | | | SSTIKYASSVQGRFTVSRDNAKKSVFLQ |
| | | | | | | | | | MNSLRDEDTAVYYCARELDSETYYNYNS |
| | | | | | | | | | LDVWGQGTTVTVSS |
| mAb-74 | Ab | SARS-CoV2 | 229E, HKU1, | | | S; Unk | B-cells; SARS-CoV1 | 943 | QVQLQESGPGLVRPSETLSLTCTVSRGSI |
| | | | NL63, OC43, | | | | Human Patient | | SSSYWSWIRQPPGKGLEWIGFMYYSGS |
| | | | SARS-CoV1 | | | | | | TNYNPSLKSRVTISLDTSKNQFSLKLSSVT |
| | | | | | | | | | AADTAVYYCAKAQGIYYRGWSYWFDP |
| | | | | | | | | | WGQGTLVTVSS |
| mAb-75 | Ab | 229E, HKU1 | OC43 | | | S; Unk | B-cells; SARS-CoV1 | 944 | EVQLVQSGAEVKKPGASVKISCKASGYA |
| | | (weak), | | | | | Human Patient | | FRNNYINWIRQAPGQGLEWMGIINPSA |
| | | NL63, SARS- | | | | | | | GTSTYAQKFQGRVTMTRDTSTNTVYME |
| | | CoV1 | | | | | | | ITSLRSEDTATYFCAREARRQVTQWFGE |
| | | (weak), | | | | | | | FWGPYNWFDPWGQGTLVTVSS |
| | | SARS-CoV2 | | | | | | | |
| mAb-76 | Ab | SARS-CoV1, | 229E, HKU1, | | | S; Unk | B-cells; SARS-CoV1 | 945 | QVQLQQWGAGLLKPSETLSLTCGVYGV |
| | | SARS-CoV2 | NL63, OC43 | | | | Human Patient | | SFSDYYWSWIRQPPGKGLEWIGEINHS |
| | | | | | | | | | GITNYNPSLKSRVTISVDTSKNQFSLKLSS |
| | | | | | | | | | VTAADTAVYYCARGLISYTLWLRESYFDY |
| | | | | | | | | | WGQGTLVTVSS |
| mAb-77 | Ab | SARS-CoV1, | 229E, HKU1, | | | S; Unk | B-cells; SARS-CoV1 | 946 | QVQLVQSGAEVKKPGSSVKVSCKASGG |
| | | SARS-CoV2 | NL63, OC43 | | | | Human Patient | | SLRDYAISWVRQAPGQGPEWMGGIMP |
| | | | | | | | | | IFGTAGYAQKFQGRVKFTADESATTAYM |
| | | | | | | | | | ELTGLRSEDSAVYFCARDPSILNTGNHH |
| | | | | | | | | | WYDLDIWGQGTTVTVSS |
| mAb-78 | Ab | SARS-CoV2 | 229E, HKU1, | | | S; Unk | B-cells; SARS-CoV1 | 947 | QVQLVQSGAEVKKPGASVKVSCKASGY |
| | | | NL63, OC43, | | | | Human Patient | | TFTNDGFVWVRQAPGQVPEWMGWIS |
| | | | SARS-CoV1 | | | | | | VHTGDTIYAQRFQGRVTMTTDTSTRTSY |
| | | | | | | | | | MELMSLRSDDTAVYYCARDYGDGPPD |
| | | | | | | | | | HWGQGTLVTVSS |
| mAb-79 | Ab | SARS-CoV1, | 229E, HKU1, | | | S; Unk | B-cells; SARS-CoV1 | 948 | QVQLVQSGPEVKKPGSSVRVSCKVSGG |
| | | SARS-CoV2 | NL63, OC43 | | | | Human Patient | | PFSSYGVSWARQAPEKGLEWMGGVLPI |
| | | | | | | | | | FGTVGYVHKFQGRVTITADESTSTVYMA |
| | | | | | | | | | LSSLRSEDTAVYYCVLDTTMSHPHNWY |
| | | | | | | | | | GMDVWGHGTTVTVSS |
| mAb-8 | Ab | SARS-CoV1, | 229E, HKU1, | | | S; Unk | B-cells; SARS-CoV1 | 949 | QVQLVQSGAEVKKPGSSMKLSCKASGI |
| | | SARS-CoV2 | NL63, OC43 | | | | Human Patient | | NFRSYSFSWVRQAPGQGLEWMGGVIP |
| | | | | | | | | | YFPTANYAEKFRGRVTATADESTGTVYLE |
| | | | | | | | | | MSSLRSEDTAVYYCASEYFDGRSYHSFC |
| | | | | | | | | | GLDVWGQGTLVTVSS |
| mAb-80 | Ab | 229E, HKU1 | OC43 | | | S; Unk | B-cells; SARS-CoV1 | 950 | QVQLVQSGAEVKKPGSSVKVSCKASGG |
| | | (weak), | | | | | Human Patient | | TLSDYAISWVRQAPGQGLEWMGGIMP |
| | | NL63, SARS- | | | | | | | VFGSPGYAEIFQGRLTITADESRSTAYME |
| | | CoV1 | | | | | | | LTSLRSEDTAVYYCARDPSILNTGPHHW |
| | | (weak), | | | | | | | YDLDIWGPGTTVTVSS |
| | | SARS-CoV2 | | | | | | | |
| mAb-81 | Ab | SARS-CoV2 | 229E, HKU1, | | | S; Unk | B-cells; SARS-CoV1 | 951 | EVQLVQSGAEVKKPGSSMRVSCRVSGG |
| | | | NL63, OC43, | | | | Human Patient | | TFITHAMSWVRQAPGQGPEWMGGIV |
| | | | SARS-CoV1 | | | | | | PLFGRASYAQPSQTRVQITADESTSTVYL |
| | | | | | | | | | EVPSLTSEDTAVYYCVRDSEPYTATRSQN |
| | | | | | | | | | HYWYDMDVWGQGTTVTVSS |
| mAb-82 | Ab | HKU1, SARS- | 229E, NL63, | | | S; Unk | B-cells; SARS-CoV1 | 952 | EVQLVESGPGLVKPSATLSLTCTVSGGSI |
| | | CoV1, SARS- | OC43 | | | | Human Patient | | NNYYWTWVRQSAGKGLEWIGRINTSG |
| | | CoV2 | | | | | | | STNYNASLKSRVTMSIDTSKNEFSLRLSS |
| | | | | | | | | | VTAADTAVYYCAREFGVRFLDRSLFGAM |
| | | | | | | | | | DVWGHGATVTVSS |
| mAb-83 | Ab | SARS-CoV2 | 229E, HKU1, | | | S; Unk | B-cells; SARS-CoV1 | 953 | QVQLVESGGGVVQPGRSLRLSCAASGF |
| | | | NL63, OC43, | | | | Human Patient | | TFSAFAMHWVRQAPGKGLEWVTIISYD |
| | | | SARS-CoV1 | | | | | | GSNEYYADSVQGRFSISRDNSKNTLFLQ |
| | | | | | | | | | MSSLRTEDTAIYYCARAGGYLSAFDIWG |
| | | | | | | | | | QGTTVTVSS |
| mAb-84 | Ab | SARS-CoV1, | 229E, HKU1, | | | S; Unk | B-cells; SARS-CoV1 | 954 | EVQLVQSGAEVKKPGSSVKVSCKVSGGT |
| | | SARS-CoV2 | NL63, OC43 | | | | Human Patient | | FSNYAISWLRQAPGQGPEWMGGIIPAL |
| | | | | | | | | | SKVGYAGKFQARLTFSADELKTTVYMDL |
| | | | | | | | | | SSLTSEDTAVYYCARDPSFLNAGNHFYYD |
| | | | | | | | | | FDVWGQGTMVTVSS |
| mAb-85 | Ab | SARS-CoV2 | 229E, HKU1, | | | S; Unk | B-cells; SARS-CoV1 | 955 | QVQLQESGPGLVKPSGTLSLTCAVSGGS |
| | | | NL63, OC43, | | | | Human Patient | | ITSRDWWSWVRQPPGKGLEWIGEVYH |
| | | | SARS-CoV1 | | | | | | SGSTSYNPSLKSRVTISVDKSKNEFSLKLS |
| | | | | | | | | | SVTAADTAVYYCARAGNIVVMPAAQYY |
| | | | | | | | | | FDYWGQGTLVTVSS |
| mAb-86 | Ab | SARS-CoV1, | 229E, HKU1, | | | S; Unk | B-cells; SARS-CoV1 | 956 | EVQLVESGGSVVQPGTSLKLSCAGSAGL |
| | | SARS-CoV2 | NL63, OC43 | | | | Human Patient | | TITRYAMHWVRQAPGKGLEWVALVSH |
| | | | | | | | | | DGIHIGYSDSVRGRFTISRDNSRNTLYLQ |
| | | | | | | | | | MDGLRPEDTAVYYCVRDDVLQHSRPSG |
| | | | | | | | | | PGYFVSWGQGTLVTVSS |
| mAb-87 | Ab | SARS-CoV2 | 229E, HKU1, | | | S; Unk | B-cells; SARS-CoV1 | 957 | QVQLQESGPGLVKPSETLSLTCTVSGGS |
| | | | NL63, OC43, | | | | Human Patient | | MNHYYWSWIRQPPGKGLEGIGYTYYSG |
| | | | SARS-CoV1 | | | | | | STNYNPSLKSRVTISVDASKNQFSLRLSSV |
| | | | | | | | | | TAADTAVYYCARGSQIDLRGGLGATFFD |
| | | | | | | | | | YWGQGTLVTVSS |
| mAb-88 | Ab | SARS-CoV2 | 229E, HKU1, | | | S; Unk | B-cells; SARS-CoV1 | 958 | QVQLVESGAEVKKPGSSVKISCKISGDTF |
| | | | NL63, OC43, | | | | Human Patient | | STNAISWLRQAPGREPEWMGGIVPLVG |
| | | | SARS-CoV1 | | | | | | PASYAQRPQGRLTITADEFTNTAYLELNS |
| | | | | | | | | | LRSEDTATYYCARDSDPYTATRRHNHYW |
| | | | | | | | | | YAMDVWGQGTTVTVSS |
| mAb-89 | Ab | SARS-CoV1, | 229E, HKU1, | | | S; Unk | B-cells; SARS-CoV1 | 959 | EVQLLESGGGLVQPGGSLRLSCAASGFIF |
| | | SARS-CoV2 | NL63, OC43 | | | | Human Patient | | KNEPMNWIRQFPGKGLEWISNIRDNG |
| | | | | | | | | | NDVYYADSVKGRFTVSRDNAKNSLYLQ |
| | | | | | | | | | MNSLRDDDTALYYCVRDTDWAFDSWG |
| | | | | | | | | | QGTLVTVSS |
| mAb-9 | Ab | SARS-CoV1, | 229E, HKU1, | | | S; Unk | B-cells; SARS-CoV1 | 960 | QVQLVQSGAEVKKPGASVKVSCKASGY |
| | | SARS-CoV2 | NL63, OC43 | | | | Human Patient | | TFSSYDINWVRQATGQGLEWMGWMS |
| | | | | | | | | | PNTGDTGYAHKFQGRVRMTSNTSISTA |
| | | | | | | | | | YMELNSLTSEDTAVYYCARRGNNFGYYY |
| | | | | | | | | | YYTVDVWGQGTTVTVSS |
| mAb-90 | Ab | HKU1, SARS- | 229E, NL63, | | | S; Unk | B-cells; SARS-CoV1 | 961 | QVQLVQSGADVKKPGASVKVSCKASGY |
| | | CoV1, SARS- | OC43 | | | | Human Patient | | TFTSYYMHWVRQAPGQGLEWLGVIHP |
| | | CoV2 | | | | | | | SGGSTTFAQKFQGRVTMTRDTSTSTVY |
| | | | | | | | | | MELSSLRSEDTAVYYCARVLAGSSHEW |
| | | | | | | | | | QLTHDAFDIWGQGTTVTVSS |
| mAb-91 | Ab | HKU1 | 229E, NL63, | | | S; Unk | B-cells; SARS-CoV1 | 962 | EVQLLESGGGLVQPGGSLRLSCAASGFT |
| | | (weak), | OC43 | | | | Human Patient | | FSSYAMSWVRQAPGKGLEWVSGIDGG |
| | | SARS-CoV1 | | | | | | | GGSSYYADSVRGRFTVSRDNSKNMLHL |
| | | (weak), | | | | | | | QMNSLRADDTAVYFCAKGDWIRYFDW |
| | | SARS-CoV2 | | | | | | | SLPISFFDYWGQGALVTVSS |
| mAb-92 | Ab | SARS-CoV1, | 229E, HKU1, | | | S; Unk | B-cells; SARS-CoV1 | 963 | EVQLLESGPGLLKPSETLSLTCTVSGDSV |
| | | SARS-CoV2 | NL63, OC43 | | | | Human Patient | | SSGNFYWSWVRRPPGKALEWIAYSHYT |
| | | | | | | | | | GGTNSDPSFMGRVTMSIDPSRNQFSLR |
| | | | | | | | | | LTSVAAADTAVYYCARTTSPLTYSGHWP |
| | | | | | | | | | LFDYWGQGSLVTVSS |
| mAb-93 | Ab | SARS-CoV1, | 229E, HKU1, | | | S; Unk | B-cells; SARS-CoV1 | 964 | QVQLVQSGDEMKKPGSSVKVSCKASGD |
| | | SARS-CoV2 | NL63, OC43 | | | | Human Patient | | TFSTHAISWVRQAPGQGPEWMGGIIPL |
| | | | | | | | | | FGTASYAQTSQSRVKITADESTSTAYMEL |
| | | | | | | | | | SSLTSEDTAVYYCVRDSDPYTATSRNNHY |
| | | | | | | | | | WYGMDVWGQGTTVTVSS |
| mAb-94 | Ab | SARS-CoV2 | 229E, HKU1, | | | S; Unk | B-cells; SARS-CoV1 | 965 | QVQLVQSGAEVKKPGSSVKVSCKASGG |
| | | | NL63, OC43, | | | | Human Patient | | MFTDYAISWVRQAPGQRLEWMGGIM |
| | | | SARS-CoV1 | | | | | | PGLGSPAYAQIFRDRATISADVSTSTAYL |
| | | | | | | | | | ELTSLKPEDTAVYYCARDPSILNTGNHH |
| | | | | | | | | | WYDLDIWGQGTTVTVSS |
| mAb-95 | Ab | SARS-CoV1, | 229E, HKU1, | | | S; Unk | B-cells; SARS-CoV1 | 966 | QVQLVQSGAEVKKPGSSVKVSCKASGG |
| | | SARS-CoV2 | NL63, OC43 | | | | Human Patient | | MFTDYAISWVRQAPGQRPEWMGGIM |
| | | | | | | | | | PGLGSPAYAQIFRGRATISADLSTSTAYLE |
| | | | | | | | | | LTSLKPEDTAVYYCARDPSILNTGNHHW |
| | | | | | | | | | YDLDIWGQGTMVTVSS |
| mAb-96 | Ab | SARS-CoV2 | 229E, HKU1, | | | S; Unk | B-cells; SARS-CoV1 | 967 | QVTLKESGGGLVQPGGSLRLSCAASGFT |
| | | | NL63, OC43, | | | | Human Patient | | VNSYGMSWVRQAPGKGLEWVSGFKSR |
| | | | SARS-CoV1 | | | | | | SDRPDYAESVKGRFTISRDTSKNTVYLE |
| | | | | | | | | | MIGLRAEDTATYYCVRMDWMEWMKY |
| | | | | | | | | | YFDSWGQGALVTVSS |
| mAb-97 | Ab | SARS-CoV1 | 229E, HKU1, | | | S; Unk | B-cells; SARS-CoV1 | 968 | QVQLVQSGAEVKKPGSSVKVSCKASGG |
| | | (weak), | NL63, OC43 | | | | Human Patient | | MISDYAISWVRQAPGQRLEWMGGIMP |
| | | SARS-CoV2 | | | | | | | AFGSPGYAQIFRGRATISADVSTNTAYLE |
| | | | | | | | | | LTSLNPDDTAVYYCARDPSILNTGNHHW |
| | | | | | | | | | YDLDMWGQGTMVTVSS |
| mAb-98 | Ab | SARS-CoV1, | 229E, HKU1, | | | S; Unk | B-cells; SARS-CoV1 | 969 | QVQLVESGGSVVQPGTSLKLSCAGSAGL |
| | | SARS-CoV2 | NL63, OC43 | | | | Human Patient | | TITRYAMHWVRQAPGKGLEWVALVSH |
| | | (weak) | | | | | | | DGIHIGYSDSVRGRFTISRDNSRNTLYLQ |
| | | | | | | | | | MDGLRPEDTAVYYCVRDDVLQHSRPSG |
| | | | | | | | | | PGYFVSWGQGTLVTVSS |
| mAb-99 | Ab | SARS-CoV2 | 229E, HKU1, | | | S; Unk | B-cells; SARS-CoV1 | 970 | QVQLQESGPGLVKPSGTLSLTCAVSGAS |
| | | (weak) | NL63, OC43, | | | | Human Patient | | VSSDHWWSWVRQSPGKGLEWIGEVY |
| | | | SARS-CoV1 | | | | | | HSGSTNYNPSLKSRVTISLDQSNNQFSLK |
| | | | | | | | | | LTSVTAADTAIYYCATMWGGLCTASNCY |
| | | | | | | | | | GNPMDVWGQGTTVTVSS |
| mBG17 | Ab | SARS-CoV1, | 229E | | | N | Immunised Mouse | 971 | EVKLEESGGGLVQPGGSMKFSCVASGF |
| | | SARS-CoV2, | | | | | | | TFSDYWMNWVRQSPDKGLEWVAEIRL |
| | | NL63 | | | | | | | KSNNYATHYAASVKGRFTISRDDSKSSVY |
| | | (weak) | | | | | | | LQMNNLRAEDSGIYYCTRSAMDYWGQ |
| | | | | | | | | | GTSVTVSS |
| mBG21 | Ab | SARS-CoV1, | 229E | | | N | Immunised Mouse | 972 | QIQLVQSGPELKKPGETVKISCKASGYTF |
| | | SARS-CoV2, | | | | | | | TDYSMHWVKQAPGKGSKWMGWINTE |
| | | NL63 | | | | | | | TGEPTYADDFKGRFAFSLETSASTAYLQI |
| | | (weak) | | | | | | | NNLKNEDTATYFCALRRWGQGTLVTVS |
| | | | | | | | | | S |
| mBG22 | Ab | SARS-CoV1, | 229E | | | N | Immunised Mouse | 973 | QIQLVQSGPELKKPGETVKISCKASGYTF |
| | | SARS-CoV2, | | | | | | | TDYSMHWVKQAPGKGSKWMGWINTE |
| | | NL63 | | | | | | | TGEPTYADDFKGRFAFSLETSASTAYLQI |
| | | (weak) | | | | | | | NNLKNEDTATYFCALRRWGQGTLVTVS |
| | | | | | | | | | A |
| mBG57 | Ab | SARS-CoV1, | 229E | | | N | Immunised Mouse | 974 | QIQLVQSGPELKKPGETVKISCKASGYTF |
| | | SARS-CoV2, | | | | | | | TDYSMHWVKQAPGKGSKWMGWINTE |
| | | NL63 | | | | | | | TGEPTYADDFKGRFAFSLETSASTAYLQI |
| | | (weak) | | | | | | | NNLKNEDTATYFCALRRWGQGTLVTVS |
| | | | | | | | | | A |
| mBG67 | Ab | SARS-CoV1, | 229E | | | N | Immunised Mouse | 975 | EVQLVESGGGLVQPGGSLKLSCAASGFT |
| | | SARS-CoV2, | | | | | | | FSNYGMSWVRQTPDKRLELVATINRNG |
| | | NL63 | | | | | | | GSTYYLDSVKVRFTISRDNAKSTLFLQLSS |
| | | (weak) | | | | | | | LKSDDTAMYYCARIYDFDEDYFDVWGA |
| | | | | | | | | | GTTVTVSS |
| MD17 | Ab | SARS-CoV2 | | SARS-CoV2 | | S; RBD | Phage Display | | ND |
| | | | | (weak) | | | Library (Antibody, | | |
| | | | | | | | human, immune - | | |
| | | | | | | | CoV2) | | |
| MD29 | Ab | SARS-CoV2 | | SARS-CoV2 | | S; RBD | Phage Display | | ND |
| | | | | (weak) | | | Library (Antibody, | | |
| | | | | | | | human, immune - | | |
| | | | | | | | CoV2) | | |
| MD45 | Ab | SARS-CoV2 | | SARS-CoV2 | | S; RBD | Phage Display | | ND |
| | | | | | | | Library (Antibody, | | |
| | | | | | | | human, immune - | | |
| | | | | | | | CoV2) | | |
| MD47 | Ab | SARS-CoV2 | | SARS-CoV2 | | S; RBD | Phage Display | | ND |
| | | | | (weak) | | | Library (Antibody, | | |
| | | | | | | | human, immune - | | |
| | | | | | | | CoV2) | | |
| MD62 | Ab | SARS-CoV2 | | SARS-CoV2 | | S; RBD | Phage Display | | ND |
| | | | | | | | Library (Antibody, | | |
| | | | | | | | human, immune - | | |
| | | | | | | | CoV2) | | |
| MD63 | Ab | SARS-CoV2 | | | SARS-CoV2 | S; RBD | Phage Display | | ND |
| | | | | | | | Library (Antibody, | | |
| | | | | | | | human, immune - | | |
| | | | | | | | CoV2) | | |
| MD65 | Ab | SARS-CoV2 | | SARS-CoV2 | | S; RBD | Phage Display | | ND |
| | | | | | | | Library (Antibody, | | |
| | | | | | | | human, immune - | | |
| | | | | | | | CoV2) | | |
| MD67 | Ab | SARS-CoV2 | | SARS-CoV2 | | S; RBD | Phage Display | | ND |
| | | | | | | | Library (Antibody, | | |
| | | | | | | | human, immune - | | |
| | | | | | | | CoV2) | | |
| mNb6 | Nb | SARS-CoV2 | | SARS-CoV2 | | S; RBD | Yeast Display | | ND |
| | | | | | | | Library (scFv, | | |
| | | | | | | | human) | | |
| mNb6-tri | Nb | SARS-CoV2 | | SARS-CoV2 | | S; RBD | Yeast Display | | ND |
| | | | | | | | Library (scFv, | | |
| | | | | | | | human) | | |
| MnCit3p1_ | Ab | SARS-CoV2 | | SARS-CoV2 | | S; RBD | B-cells; SARS-CoV2 | 976 | EVQLLESGGGLVQPGGSLRLSCAASGFT |
| G9 | | | | | | | Human Patient | | FRNYAMTWVRQAPGKGLEWVSGISDS |
| | | | | | | | | | GDRTYNADSVKGRFSISRDNSKNTLHLQ |
| | | | | | | | | | MNSLRAEDTAVYYCALASGSYFGGANY |
| | | | | | | | | | WGQGTLVTVSS |
| MnC2t1p1_ | Ab | SARS-CoV2 | | SARS-CoV2 | | S; RBD | B-cells; SARS-CoV2 | 977 | EVQLVESGGGLVQPGGSLRLSCAASGFT |
| A3 | | | | | | | Human Patient | | VSSNYMSWVRQAPGRGLEWVSVIYSG |
| | | | | | | | | | GSTFYADSVKGRFTISRDNSKNTLYLQM |
| | | | | | | | | | NSLRPEDTAVYYCATGARFGESPFDYW |
| | | | | | | | | | GQGTLVTVSS |
| MnC2t1p1_ | Ab | SARS-CoV2 | | SARS-CoV2 | | S; RBD | B-cells; SARS-CoV2 | 978 | EVQLVESGGGLVQPGGSLRLSCAASGFT |
| C5 | | | | | | | Human Patient | | VSSNYMSWVRQAPGKGLEWVSVIYSG |
| | | | | | | | | | GSTYYADSVKGRFTISRDNSKNTLYLQM |
| | | | | | | | | | NSLRAEDTAVYYCATGARFGESPFDYW |
| | | | | | | | | | GQGTLVTVSS |
| MnC2t2p1_ | Ab | SARS-CoV2 | | SARS-CoV2 | | S; RBD | B-cells; SARS-COV2 | 979 | QVQLVQSGAEVKKPGSSVKVSCKASGG |
| C11 | | | | | | | Human Patient | | TFSRYTIIWVRQAPGQGLEWMGRIIPIL |
| | | | | | | | | | DIANYAQKFQGRVTITADKSTSTAYMEL |
| | | | | | | | | | SSLRSEDTAVYYCAREGGLDYFGSRNSG |
| | | | | | | | | | WTYTWFDPWGQGTLVTVSS |
| MnC4t1p1_ | Ab | SARS-CoV2 | | SARS-CoV2 | | S; RBD | B-cells; SARS-CoV2 | 980 | QLQVQESGPGLVKPSETLSLTCTVSGASI |
| A10 | | | | | | | Human Patient | | SSNHYFWGWIRQPPGKGLAWIGSMHY |
| | | | | | | | | | SGSTYYNPSLKSRVTISVDTSKNQLSLKLS |
| | | | | | | | | | SVTAADTAVYYCARGVNYYDRNGYYRN |
| | | | | | | | | | DGFDIRGQGTMVTVSS |
| MnC4t1p1_ | Ab | SARS-CoV2 | | SARS-CoV2 | | S; RBD | B-cells; SARS-CoV2 | 981 | EVQLVESGGGLVQPGGSLRLSCAASGFT |
| A11 | | | | | | | Human Patient | | FSSYSMNWVRQAPGKGLEWVSYISSSS |
| | | | | | | | | | NTRYYTDSVMGRFTISRDNAKNSLFLQ |
| | | | | | | | | | MNSLRAEDTAVYYCASSKGFCSGGSCSD |
| | | | | | | | | | YWGQGTLVTVSS |
| MnC4t2p1_ | Ab | SARS-CoV2 | | SARS-CoV2 | | S; RBD | B-cells; SARS-CoV2 | 982 | EVQLVESGGHLVQPGRSLRLSCAASGFT |
| B3 | | | | | | | Human Patient | | FDDYAMHWVRQVPGKGLEWVSGISW |
| | | | | | | | | | NGGILDYADSVKGRFTISRDNAKNSLYL |
| | | | | | | | | | HMRSLRTDDTALYYCAKDLRRQDYYAD |
| | | | | | | | | | WYFDLWGRGTLVTVSS |
| MnC4t2p1_ | Ab | SARS-CoV2 | | SARS-CoV2 | | S; RBD | B-cells; SARS-CoV2 | 983 | QLQVQESGPGLVKPSETLSLTCTVSGASI |
| D10 | | | | | | | Human Patient | | SSNHYFWGWIRQPPGKGLAWIGSMHY |
| | | | | | | | | | SGSTYYNPSLKSRVTISVDTSKNQLSLKLS |
| | | | | | | | | | SVTAADTAVYYCARGVNYYDRNGYYRN |
| | | | | | | | | | DGFDIRGQGTMVTVSS |
| MnC4t2p1_ | Ab | SARS-CoV2 | | SARS-CoV2 | | S; RBD | B-cells; SARS-CoV2 | 984 | EVQLVESGGHLVQPGRSLRLSCAASGFT |
| E6 | | | | | | | Human Patient | | FDDYAMHWVRQVPGKGLEWVSGISW |
| | | | | | | | | | NGGILGYADSVKGRFTISRDNAKNSLYL |
| | | | | | | | | | QMRSLRTDDTALYYCAKDLRRQDYYAD |
| | | | | | | | | | WYFDLWGRGTLVTVSS |
| MnC4t2p1_ | Ab | SARS-CoV2 | | SARS-CoV2 | | S; RBD | B-cells; SARS-CoV2 | 985 | QLQVQESGPGLVKPSETLSLTCTVSGASI |
| F5 | | | | | | | Human Patient | | SSNHYFWGWIRQPPGKGLAWIGSMHY |
| | | | | | | | | | SGSTYYNPSLKSRVTISVDTSKNQLSLKLS |
| | | | | | | | | | SVTAADTAVYYCARGVNYYDRNGYYRN |
| | | | | | | | | | DGFDIRGQGTMVTVSS |
| MnC4t2p2_ | Ab | SARS-CoV2 | | SARS-CoV2 | | S; RBD | B-cells; SARS-CoV2 | 986 | QVQLVQSGSELKKPGASVKISCKASGYIF |
| A4 | | | | | | | Human Patient | | INYAMNWVRQAPGQGLEWMGWINT |
| | | | | | | | | | NTGNPTYAQDFTGRFVFSLDTSLSTAYL |
| | | | | | | | | | QISSLEAEDTAVYYCAKIGSRNSLGVWG |
| | | | | | | | | | QGTLVTVSA |
| MnC5t2p1_ | Ab | SARS-CoV2 | | SARS-CoV2 | | S; RBD | B-cells; SARS-CoV2 | 987 | QMQLVQSGPEVKKPGTSVKVSCKASGF |
| G1 | | | | | | | Human Patient | | TFTSSAVQWVRQARGQRLEWIGWIVV |
| | | | | | | | | | GSGNTDYAQKFQERVTITRDVSTSTAYM |
| | | | | | | | | | ELSSLRSEDTAVYYCAAPRCSGGSCYDGF |
| | | | | | | | | | DIWGQGTMVTVSS |
| MR10 | Nb | SARS-CoV2 | | | | S; RBD | Phage Display | 988 | QVQLVESGGGLVQAGGSLRLSCAASGF |
| | | | | | | | Library (Nanobody, | | PVANTWMEWYRQAPGKEREWVAAITS |
| | | | | | | | non-immune) | | YGYRTYYADSVKGRFTISRDNAKNTVYL |
| | | | | | | | | | QMNSLKPEDTAVYYCNVKDEGATTKVY |
| | | | | | | | | | DYWGQGTQVTVSS |
| MR14 | Nb | SARS-CoV2 | | | | S; RBD | Phage Display | 989 | QVQLVESGGGLVQAGGSLRLSCAASGF |
| | | | | | | | Library (Nanobody, | | PVMYTHMHWYRQAPGKEREWVAAIV |
| | | | | | | | non-immune) | | SLGEYTTYADSVKGRFTISRDNAKNTVYL |
| | | | | | | | | | QMNSLKPEDTAVYYCNVKDWGAANKY |
| | | | | | | | | | YDYWGQGTQVTVSS |
| MR17 | Nb | SARS-CoV2 | | | | S; RBD | Phage Display | 990 | QVQLVESGGGLVQAGGSLRLSCAASGF |
| | | | | | | | Library (Nanobody, | | PVEVWRMEWYRQAPGKEREGVAAIES |
| | | | | | | | non-immune) | | YGHGTRYADSVKGRFTISRDNAKNTVYL |
| | | | | | | | | | QMNSLKPEDTAVYYCNVKDDGQLAYHY |
| | | | | | | | | | DYWGQGTQVTVSS |
| MR17_K56W | Nb | SARS-CoV2 | | | | S; RBD | Derived from MR17 | 991 | QVQLVESGGGLVQAGGSLRLSCAASGF |
| | | | | | | | | | PVEVWRMEWYRQAPGKEREGVAAIES |
| | | | | | | | | | YGWGTRYADSVKGRFTISRDNAKNTVYL |
| | | | | | | | | | QMNSLKPEDTAVYYCNVKDDGQLAYHY |
| | | | | | | | | | DYWGQGTQVTVSS |
| MR17_K99W | Nb | SARS-CoV2 | | | | S; RBD | Derived from MR17 | 992 | QVQLVESGGGLVQAGGSLRLSCAASGF |
| | | | | | | | | | PVEVWRMEWYRQAPGKEREGVAAIES |
| | | | | | | | | | YGHGTRYADSVKGRFTISRDNAKNTVYL |
| | | | | | | | | | QMNSLKPEDTAVYYCNVWDDGQLAYH |
| | | | | | | | | | YDYWGQGTQVTVSS |
| MR17_K99Y | Nb | SARS-CoV2 | | | | S; RBD | Derived from MR17 | 993 | QVQLVESGGGLVQAGGSLRLSCAASGF |
| | | | | | | | | | PVEVWRMEWYRQAPGKEREGVAAIES |
| | | | | | | | | | YGHGTRYADSVKGRFTISRDNAKNTVYL |
| | | | | | | | | | QMNSLKPEDTAVYYCNVYDDGQLAYHY |
| | | | | | | | | | DYWGQGTQVTVSS |
| MR2 | Nb | SARS-CoV2 | | | | S; RBD | Phage Display | 994 | QVQLVESGGGLVQAGGSLRLSCAASGF |
| | | | | | | | Library (Nanobody, | | PVYFSYMAWYRQAPGKEREWVAAINSE |
| | | | | | | | non-immune) | | GDSTTYADSVKGRFTISRDNAKNTVYLQ |
| | | | | | | | | | MNSLKPEDTAVYYCNVKDYGWYNSQY |
| | | | | | | | | | DYWGQGTQVTVSS |
| MR3 | Nb | SARS-CoV2 | | | | S; RBD | Phage Display | 995 | QVQLVESGGGLVQAGGSLRLSCAASGF |
| | | | | | | | Library (Nanobody, | | PVNAHFMYWYRQAPGKEREWVAAIYS |
| | | | | | | | non-immune) | | YGRTLYADSVKGRFTISRDNAKNTVYLQ |
| | | | | | | | | | MNSLKPEDTAVYYCNVKDYGAASWEYD |
| | | | | | | | | | YWGQGTQVTVSS |
| MR4 | Nb | SARS-CoV2 | | | | S; RBD | Phage Display | 996 | QVQLVESGGGLVQAGGSLRLSCAASGF |
| | | | | | | | Library (Nanobody, | | PMYAWEMAWYRQAPGKEREWVAAIR |
| | | | | | | | non-immune) | | SMGVHTHYSDSVKGRFTISRDNAKNTV |
| | | | | | | | | | YLQMNSLKPEDTAVYYCNVKDFGGHQA |
| | | | | | | | | | YYDYWGQGTQVTVSS |
| MR6 | Nb | SARS-CoV2 | | | | S; RBD | Phage Display | 997 | QVQLVESGGGLVQAGGSLRLSCAASGF |
| | | | | | | | Library (Nanobody, | | PVEDTWMEWYRQAPGKEREWVAAITS |
| | | | | | | | non-immune) | | WGFKTYYADSVKGRFTISRDNAKNTVYL |
| | | | | | | | | | QMNSLKPEDTAVYYCNVKDEGDTSASY |
| | | | | | | | | | DYWGQGTQVTVSS |
| MR7 | Nb | SARS-CoV2 | | | | S; RBD | Phage Display | 998 | QVQLVESGGGLVQAGGSLRLSCAASGF |
| | | | | | | | Library (Nanobody, | | PVNSWMEWYRQAPGKEREWVAAITSY |
| | | | | | | | non-immune) | | GYKTYYADSVKGRFTISRDNAKNTVYLQ |
| | | | | | | | | | MNSLKPEDTAVYYCNVKDEGYFSDEYDY |
| | | | | | | | | | WGQGTQVTVSS |
| MR8 | Nb | SARS-CoV2 | | | | S; RBD | Phage Display | 999 | QVQLVESGGGLVQAGGSLRLSCAASGF |
| | | | | | | | Library (Nanobody, | | PVEWAHMHWYRQAPGKEREWVAAIV |
| | | | | | | | non-immune) | | SAGHYTVYADSVKGRFTISRDNAKNTVY |
| | | | | | | | | | LQMNSLKPEDTAVYYCNVKDWGSSNQ |
| | | | | | | | | | YYDYWGQGTQVTVSS |
| Nb11 | Nb | SARS-CoV2 | | SARS-CoV2 | | S; RBD | Yeast Display | | ND |
| | | | | | | | Library (scFv, | | |
| | | | | | | | human) | | |
| Nb11-59 | Nb | SARS-CoV1, | | SARS-CoV1, | | S; RBD | Phage Display | | ND |
| | | SARS-CoV2 | | SARS-CoV2 | | | (Immunised Camel) | | |
| Nb3 | Nb | SARS-CoV2 | | SARS-CoV2 | | S; RBD | Yeast Display | | ND |
| | | | | | | | Library (scFv, | | |
| | | | | | | | human) | | |
| Nb3-bi | Nb | SARS-CoV2 | | SARS-CoV2 | | S; RBD | Yeast Display | | ND |
| | | | | | | | Library (scFv, | | |
| | | | | | | | human) | | |
| Nb3-tri | Nb | SARS-CoV2 | | SARS-CoV2 | | S; RBD | Yeast Display | | ND |
| | | | | | | | Library (scFv, | | |
| | | | | | | | human) | | |
| Nb4-43 | Nb | SARS-CoV2 | SARS-CoV1 | SARS-CoV2 | SARS-CoV1 | S; RBD | Phage Display | | ND |
| | | | | | | | (Immunised Camel) | | |
| Nb6 | Nb | SARS-CoV2 | | SARS-CoV2 | | S; RBD | Yeast Display | | ND |
| | | | | | | | Library (scFv, | | |
| | | | | | | | human) | | |
| Nb6-tri | Nb | SARS-CoV2 | | SARS-CoV2 | | S; RBD | Yeast Display | | ND |
| | | | | | | | Library (scFv, | | |
| | | | | | | | human) | | |
| NIH- | Nb | SARS-CoV2 | | | | S; RBD | Immunised Llama | 1000 | DVQLQESGGDLVQPGGSLRLSCAASGF |
| CoVnb- | | | | | | | | | TLDYYAIGWFRQAPGKEREGVSCISSSD |
| 101 | | | | | | | | | GSTYYADSVKGRFTSSRDNAKNTVYLQ |
| | | | | | | | | | MNSLKPEDTAVYYCAAVPSTYYNGSYYY |
| | | | | | | | | | TCHPGGMDYWGKGTQVTVSS |
| NIH- | Nb | SARS-CoV2 | | | | S; RBD | Immunised Llama | 1001 | DVQLQESGGGLVQPGGSLRLSCAVSGF |
| CoVnb- | | | | | | | | | TLDYYAIGWFRQAPGKEREGVSCISSSD |
| 102 | | | | | | | | | GSTYYADSVKGRFTSSRDNAKNTVYLQ |
| | | | | | | | | | MNSLKPEDTAVYYCAAVPSTYYSGTYYY |
| | | | | | | | | | NCHPGGMDYWGKGTQVTVSS |
| NIH- | Nb | SARS-CoV2 | | | | S; RBD | Immunised Llama | 1002 | DVQLQESGGGLVQPGGSLRLSCAASGL |
| CoVnb- | | | | | | | | | TLDYYTIGWFRQAPGKEREGVSCISSSD |
| 103 | | | | | | | | | DSTYYADSVKGRFTISRDNAKNTVYLQM |
| | | | | | | | | | NSLKPEDTAVYYCATAPGTYYKGSYYPM |
| | | | | | | | | | CHYYGMDYWGKGTQVTVSS |
| NIH- | Nb | SARS-CoV2 | | | | S; RBD | Immunised Llama | 1003 | DVQLQESGGGLVQPGGSLRLSCAVSGF |
| CoVnb- | | | | | | | | | TLDYYAIGWFRQAPGKEREGVACISSSD |
| 104 | | | | | | | | | GTTYYADSVKGRFTISRDNAKNTVYLQM |
| | | | | | | | | | NSLKPEDTAVYYCATRPLTYYSGSYYTTCS |
| | | | | | | | | | DYGMDYWGKGTLVTVSS |
| NIH- | Nb | SARS-CoV2 | | | | S; RBD | Immunised Llama | 1004 | DVQLQESGGGLVQPGGSLRLSCAASGF |
| CoVnb- | | | | | | | | | TLDYYAIGWFRQAPGKEREGVSCISNSD |
| 105 | | | | | | | | | GSTYYADSVKGRFTTSRDNAKNTVYLQ |
| | | | | | | | | | MNSLKPEDTAVYYCAAVPSTYYSGSYYYT |
| | | | | | | | | | CHPGGMDYWGKGTQVTVSS |
| NIH- | Nb | SARS-CoV2 | | | | S; RBD | Immunised Llama | 1005 | DVQLQESGGGLVQSGGSLRLSCAASGF |
| CoVnb- | | | | | | | | | TLDYYAIGWFRQAPGKEREGVSCISNSD |
| 106 | | | | | | | | | GSTYYADSVKGRFTTSRDNAKNTVYLQ |
| | | | | | | | | | MNSLKPEDTAVYYCAAVPSTYYSGSYYYT |
| | | | | | | | | | CHPGGMDYWGKGTQVTVSS |
| NIH- | Nb | SARS-CoV2 | | | | S; RBD | Immunised Llama | 1006 | DVQLQESGGGLVQPGGSLRLSCAASGF |
| CoVnb- | | | | | | | | | TLDYYAIGWFRQAPGKEREGVSCISNSD |
| 107 | | | | | | | | | GSTYYADSVKGRFTTSRDNAKNTVYLQ |
| | | | | | | | | | MNSLKPEDTAVYYCAAVPSTYYSGSYYYT |
| | | | | | | | | | CHPGGMDYWGKGTLVTVSS |
| NIH- | Nb | SARS-CoV2 | | | | S; RBD | Immunised Llama | 1007 | DVQLQESGGGLVQPGGSLRLSCAASGF |
| CoVnb- | | | | | | | | | TLDYYAIGWFRQAPGKEREGVSCISNSG |
| 108 | | | | | | | | | GSTYYADSVKGRFTTSRDNAKNTVYLQ |
| | | | | | | | | | MNSLKPEDTAVYYCAAVPSTYYSGSYYYT |
| | | | | | | | | | CHPGGMDYWGKGTQVTVSS |
| NIH- | Nb | SARS-CoV2 | | | | S; RBD | Immunised Llama | 1008 | DVQLQESGGGLVQSGGSLRLSCAASGF |
| CoVnb- | | | | | | | | | TLDYYAIGWFRQAPGKEREGVSCITNSD |
| 109 | | | | | | | | | GSTYYADSVKGRFTTSRDNAKNTVYLQ |
| | | | | | | | | | MNSLKPEDTAVYYCASFPSTYYSGSYYYT |
| | | | | | | | | | CHPGGMDYWGKGTQVTVSS |
| NIH- | Nb | SARS-CoV2 | | | | S; RBD | Immunised Llama | 1009 | DVQLQESGGGLVQPGGSLRLSCAASGF |
| CoVnb- | | | | | | | | | TLDYYAIGWFRQAPGKEREGVSCISSSD |
| 110 | | | | | | | | | GSTYYADSVKGRFTISRDNAKNTVYLQM |
| | | | | | | | | | NSLKPDDTAVYYCAAALSEGGYTIDGSS |
| | | | | | | | | | WCYHSVYGMDYWGKGTQVTVSS |
| NIH- | Nb | SARS-CoV2 | | | | S; RBD | Immunised Llama | 1010 | DVQLQESGGGSVEAGGSLRLSCAASGV |
| CoVnb- | | | | | | | | | TLDYYAIGWFRQAPGKEREGVSCISSSD |
| 111 | | | | | | | | | GSTYYADSVKGRFTTSRDNAKNTVYLQ |
| | | | | | | | | | MNSLKPEDTADYYCAAVPSTYYSGTYYY |
| | | | | | | | | | NCHPGAMHYWGKGTQVTVSS |
| NIH- | Nb | SARS-CoV2 | | | | S; RBD | Immunised Llama | 1011 | DVQLQESGGGLVQPGGSLRLSCAASGL |
| CoVnb- | | | | | | | | | TLDYYAIGWFRQAPGKEREGVSCISSSD |
| 112 | | | | | | | | | GSTYYADSVKGRFTTSRDNAKNTVYLQ |
| | | | | | | | | | MNSLKPEDTAVYYCAAVPSTYYSGTYYY |
| | | | | | | | | | TCHPGGMDYWGKGTQVTVSS |
| NIH- | Nb | SARS-CoV2 | | | | S; RBD | Immunised Llama | 1012 | DVQLQESGGGLVQPGGSLRLSCAASGL |
| CoVnb- | | | | | | | | | TLDYYAIGWFRQAPGKEREGVSCISSSD |
| 113 | | | | | | | | | GSTYYADSVKGRFTTSRDNAKNTVYLQ |
| | | | | | | | | | MNSLKPEDTAVYYCAAVPSTYYSGTYYY |
| | | | | | | | | | TCHPGGMDYWGKGTLVTVSS |
| P2B-2F6 | Ab | SARS-CoV2 | SARS-CoV1 | SARS-CoV2 | | S; RBD | B-cells; SARS-CoV2 | 1013 | QVQLQESGPGLVKPSETLSLTCTVSGYSI |
| | | | | | | | Human Patient | | SSGYYWGWIRQPPGKGLEWIGSIYHSG |
| | | | | | | | | | STYYNPSLKTRVTISVDTSKNQFSLKLSSV |
| | | | | | | | | | TAADTAVYYCARAVVGIVVVPAAGRRAF |
| | | | | | | | | | DIWGQGTMVTVSS |
| S110 | Ab | SARS-CoV1, | | SARS-CoV1 | | S; | B-cells; SARS-CoV1 | | ND |
| | | SARS-CoV2 | | | | RBD + non- | Human Patient | | |
| S124 | Ab | SARS-CoV1, | | SARS-CoV1 | | S; RBD | B-cells; SARS-CoV1 | | ND |
| | | SARS-CoV2 | | | | | Human Patient | | |
| S303 | Ab | SARS-CoV1, | | SARS-CoV1 | | S; RBD | B-cells; SARS-CoV1 | | ND |
| | | SARS-CoV2 | | | | | Human Patient | | |
| S304 | Ab | SARS-CoV1, | | SARS-CoV1, | | S; RBD | B-cells; SARS-CoV1 | | ND |
| | | SARS-CoV2 | | SARS-CoV2 | | | Human Patient | | |
| S306 | Ab | SARS-CoV1, | | SARS-CoV1 | SARS-CoV2 | S; non- | B-cells; SARS-CoV1 | | ND |
| | | SARS-CoV2 | | | | RBD | Human Patient | | |
| S309 | Ab | SARS-CoV1, | | SARS-CoV2 | | S; RBD | B-cells; SARS-CoV1 | 1014 | QVQLVQSGAEVKKPGASVKVSCKASGY |
| | | SARS-CoV2 | | and SARS- | | | Human Patient | | PFTSYGISWVRQAPGQGLEWMGWIST |
| | | | | CoV1 | | | | | YNGNTNYAQKFQGRVTMTTDTSTTTGY |
| | | | | | | | | | MELRRLRSDDTAVYYCARDYTRGAWFG |
| | | | | | | | | | ESLIGGFDNWGQGTLVTVSS |
| S310 | Ab | SARS-CoV1, | | SARS-CoV1 | | S; non- | B-cells; SARS-CoV1 | | ND |
| | | SARS-CoV2 | | | | RBD | Human Patient | | |
| S315 | Ab | SARS-CoV1, | | SARS-CoV2 | | S; RBD | B-cells; SARS-CoV1 | | ND |
| | | SARS-CoV2 | | and SARS- | | | Human Patient | | |
| | | | | CoV1 | | | | | |
| Sb#1 | Nb | SARS-CoV2 | | | | S; RBD | Phage Display | 1015 | QVQLVESGGGLVQAGGSLRLSCAASGF |
| | | | | | | | Library (Nanobody, | | PVRKANMHWYRQAPGKEREWVAAIM |
| | | | | | | | non-immune) | | SKGEQTVYADSVEGRFTISRDNAKNTVY |
| | | | | | | | | | LQMNSLKPEDTAVYYCRVFVGWHYFG |
| | | | | | | | | | QGTQVTVS |
| Sb#10 | Nb | SARS-CoV2 | | | | S; RBD | Phage Display | 1016 | QVQLVESGGGLVQAGGSLRLSCAASGF |
| | | | | | | | Library (Nanobody, | | PVQSHYMRWYRQAPGKEREWVAAIES |
| | | | | | | | non-immune) | | TGHHTAYADSVKGRFTISRDNAKNTVYL |
| | | | | | | | | | QMNSLKPEDTAVYYCTVYVGYEYHGQG |
| | | | | | | | | | TQVTVS |
| Sb#11 | Nb | SARS-CoV2 | | | | S; RBD | Phage Display | 1017 | QVQLVESGGGLVQAGGSLRLSCAASGF |
| | | | | | | | Library (Nanobody, | | PVETENMHWYRQAPGKEREWVAAIYS |
| | | | | | | | non-immune) | | HGMWTAYADSVKGRFTISRDNTKNTVY |
| | | | | | | | | | LQMNSLKPEDTAVYYCEVEVGKWYFGQ |
| | | | | | | | | | GTQVTVS |
| Sb#12 | Nb | SARS-CoV2 | | | | S; RBD | Phage Display | 1018 | QVQLVESGGGLVQAGGSLRLSCAASGF |
| | | | | | | | Library (Nanobody, | | PVKASRMYWYRQAPGKEREWVAAIQS |
| | | | | | | | non-immune) | | FGEVTWYADSVKGRFTISRDNAKNTVYL |
| | | | | | | | | | QMNSLKPEDTAVYYCYVWVGQEYWG |
| | | | | | | | | | QGTQVTVS |
| Sb#13 | Nb | SARS-CoV2 | | | | S; RBD | Phage Display | 1019 | QVQLVESGGGLVQAGGSLRLSCAASGF |
| | | | | | | | Library (Nanobody, | | PVYASNMHWYRQAPGKEREWVAAIES |
| | | | | | | | non-immune) | | QGYMTAYADSVKGRFTISRDNAKNTVY |
| | | | | | | | | | LQMNSLKPEDTAVYYCWVIVGEYYVGQ |
| | | | | | | | | | GTQVTVS |
| Sb#14 | Nb | SARS-CoV2 | | | | S; RBD | Phage Display | 1020 | QVQLVESGGGLVQAGGSLRLSCAASGF |
| | | | | | | | Library (Nanobody, | | PVQAREMEWYRQAPGKEREWVAAIKS |
| | | | | | | | non-immune) | | TGTYTAYAYSVKGRFTISRDNAKNTVYLQ |
| | | | | | | | | | MNSLKPEDTAVYYCYVYVGSSYIGQGTQ |
| | | | | | | | | | VTVS |
| Sb#15 | Nb | SARS-CoV2 | | | | S; RBD | Phage Display | 1021 | QVQLVESGGGLVQAGGSLRLSCAASGF |
| | | | | | | | Library (Nanobody, | | PVKNFEMEWYRKAPGKEREWVAAIQS |
| | | | | | | | non-immune) | | GGVETYYADSVKGRFTISRDNAKNTVYL |
| | | | | | | | | | QMNSLKPEDTAVYYCFVYVGRSYIGQGT |
| | | | | | | | | | QVTVS |
| Sb#16 | Nb | SARS-CoV2 | | | | S; RBD | Phage Display | 1022 | QVQLVESGGGLVQAGGSLRLSCAASGF |
| | | | | | | | Library (Nanobody, | | PVAYKTMWWYRQAPGKEREWVAAIES |
| | | | | | | | non-immune) | | YGIKWTRYADSVKGRFTISRDNAKNTVY |
| | | | | | | | | | LQMNSLKPEDTAVYYCIVWVGAQYHG |
| | | | | | | | | | QGTQVTVS |
| Sb#17 | Nb | SARS-CoV2 | | | | S; RBD | Phage Display | 1023 | QVQLVESGGGLVQAGGSLRLSCAASGF |
| | | | | | | | Library (Nanobody, | | PVAGRNMWWYRQAPGKEREWVAAIY |
| | | | | | | | non-immune) | | SSGTYTEYADSVKGRFTISRDNAKNTVYL |
| | | | | | | | | | QMNSLKPEDTAVYYCHVWVGSLYKGQ |
| | | | | | | | | | GTQVTVS |
| Sb#18 | Nb | SARS-CoV2 | | | | S; RBD | Phage Display | 1024 | QVQLVESGGGLVQAGGSLRLSCAASGF |
| | | | | | | | Library (Nanobody, | | PVKHARMWWYRQAPGKEREWVAAID |
| | | | | | | | non-immune) | | SHGDTTWYADSVKGRFTISRDNAKNTV |
| | | | | | | | | | YLQMNSLKPEDTAVYYCYVYVGASYWG |
| | | | | | | | | | QGTQVTVS |
| Sb#19 | Nb | SARS-CoV2 | | | | S; RBD | Phage Display | 1025 | QVQLVESGGGLVQAGGSLRLSCAASGF |
| | | | | | | | Library (Nanobody, | | PVNSHEMTWYRQAPGKEREWVAAIQS |
| | | | | | | | non-immune) | | TGTVTEYADSVKGRFTISRDNAKNTVYL |
| | | | | | | | | | QMNSLKPEDTAVYYCYVYVGSSYLGQG |
| | | | | | | | | | TQVTVS |
| Sb#2 | Nb | SARS-CoV2 | | | | S; RBD | Phage Display | 1026 | QVQLVESGGGLVQAGGSLRLSCATSGF |
| | | | | | | | Library (Nanobody, | | PVYQANMHWYRQAPGKEREWVAAIQ |
| | | | | | | | non-immune) | | SYGDGTHYADSVKGRFTISRDNAKNTVY |
| | | | | | | | | | LQMNSLKPEDTAVYYCRAVYVGMHYFG |
| | | | | | | | | | QGTQVTVS |
| Sb#20 | Nb | SARS-CoV2 | | | | S; RBD | Phage Display | 1027 | QVQLVESGGGLVQAGGSLRLSCAASGF |
| | | | | | | | Library (Nanobody, | | PVEQREMEWYRQAPGKEREWVAAIDS |
| | | | | | | | non-immune) | | NGNYTFYADSVKGRFTISRDNAKNTVYL |
| | | | | | | | | | QMNSLKPEDTAVYYCYVYVGKSYIGQGT |
| | | | | | | | | | QVTVS |
| Sb#21 | Nb | SARS-CoV2 | | | | S; RBD | Phage Display | 1028 | QVQLVESGGGLVQAGGSLRLSCAASGF |
| | | | | | | | Library (Nanobody, | | PVKHHWMFWYRQAPGKEREWVAAIK |
| | | | | | | | non-immune) | | SYGYGTEYADSVKGRFTISRDNAKNTVYL |
| | | | | | | | | | QMNSLKPEDTAVYYCFVGVGTHYAGQ |
| | | | | | | | | | GTQVTVS |
| Sb#22 | Nb | SARS-CoV2 | | | | S; RBD | Phage Display | 1029 | QVQLVESGGGLVQAGGSLRLSCAASGF |
| | | | | | | | Library (Nanobody, | | PVYAAEMEWYRQAPGKEREWVAAISS |
| | | | | | | | non-immune) | | QGTITYYADSVKGRFTISRDNAKNTVYLQ |
| | | | | | | | | | MNSLKPEDTAVYYCFVYVGKSYIGQGTQ |
| | | | | | | | | | VSVS |
| Sb#23 | Nb | SARS-CoV2 | | | | S; RBD | Phage Display | 1030 | QVQLVESGGGLVQAGGSLRLSCAASGF |
| | | | | | | | Library (Nanobody, | | PVYAAEMEWYRQAPGKEREWVAAISS |
| | | | | | | | non-immune) | | QGTITYYADSVKGRFTISRDNAKNTVYLQ |
| | | | | | | | | | MNSLKPEDTAVYYCFVYVGKSYIGQGTQ |
| | | | | | | | | | VSVS |
| Sb#25 | Nb | SARS-CoV2 | | | | S; RBD | Phage Display | 1031 | QVQLVESGGGLVQAGGSLRLSCAASGF |
| | | | | | | | Library (Nanobody, | | PVHAWEMAWYRQAPGKEREWVAAIR |
| | | | | | | | non-immune) | | SFGSSTHYADSVKGRFTISRDNAKNTVYL |
| | | | | | | | | | QMNSLKPEDTAVYYCNVKDFGTHHYAY |
| | | | | | | | | | DYWGQGTQVTVS |
| Sb#26 | Nb | SARS-CoV2 | | | | S; RBD | Phage Display | 1032 | QVQLVESGGGLVQAGGSLRLSCAASGF |
| | | | | | | | Library (Nanobody, | | PVNTWWMHWYRQAPGKEREWVAAIT |
| | | | | | | | non-immune) | | SWGFRTYYADSVKGRFTISRDNAKNTVY |
| | | | | | | | | | LQMNSLKPEDTAVYYCNVKDKGMAVQ |
| | | | | | | | | | WYDYWGQGTQVTVS |
| Sb#27 | Nb | SARS-CoV2 | | | | S; RBD | Phage Display | 1033 | QVQLVESGGGLVQAGGSLRLSCAASGF |
| | | | | | | | Library (Nanobody, | | PVYNTWMEWYRQAPGKEREWVAAITS |
| | | | | | | | non-immune) | | HGYKTYYADSVKGRFTISRDNAKNTVYL |
| | | | | | | | | | QMNSLKPEDTAVYYCNVKDEGDMFTA |
| | | | | | | | | | YDYWGQGTQVTVS |
| Sb#28 | Nb | SARS-CoV2 | | | | S; RBD | Phage Display | 1034 | QVQLVESGGGLVQAGGSLRLSCAASGF |
| | | | | | | | Library (Nanobody, | | PVYHSTMFWYRQAPGKEREWVAAIYSS |
| | | | | | | | non-immune) | | GQHTYYADSVKGRFTISRDNAKNTVYLQ |
| | | | | | | | | | MNSLKPEDTAVYYCNVKDSGQWRQEY |
| | | | | | | | | | DYWGQGTQVTVS |
| Sb#29 | Nb | SARS-CoV2 | | | | S; RBD | Phage Display | 1035 | QVQLVESGGGLVQAGGSLRLSCAASGF |
| | | | | | | | Library (Nanobody, | | PVEHEMAWYRQAPGKEREWVAAIRS |
| | | | | | | | non-immune) | | MGRKTLYADSVKGRFTISRDNAKNTVYL |
| | | | | | | | | | QMNSLKPEDTAVYYCNVKDFGYTWHEY |
| | | | | | | | | | DYWGQGTQVTVS |
| Sb#3 | Nb | SARS-CoV2 | | | | S; RBD | Phage Display | 1036 | QVQLVESGGGLVQAGGSLRLSCAASGF |
| | | | | | | | Library (Nanobody, | | PVNYKTMWWYRQAPGKEREWVAAIW |
| | | | | | | | non-immune) | | SYGHTTHYADSVKGRFTISRDNAKNTVY |
| | | | | | | | | | LQMNSLKPEDTAVYYCVVWVGHNYEG |
| | | | | | | | | | QGTQVTVS |
| Sb#30 | Nb | SARS-CoV2 | | | | S; RBD | Phage Display | 1037 | QVQLVESGGGLVQAGGSLRLSCAASGF |
| | | | | | | | Library (Nanobody, | | PVTMAWMWWYRQAPGKEREWVAAI |
| | | | | | | | non-immune) | | RSEGVRTYYADSVKGRFTISRDNAKNTV |
| | | | | | | | | | YLQMNSLKPEDTAVYYCNVKDYGQAHA |
| | | | | | | | | | YYDYWGQGTQVTVS |
| Sb#31 | Nb | SARS-CoV2 | | | | S; RBD | Phage Display | 1038 | QVQLVESGGGLVQAGGSLRLSCAASGF |
| | | | | | | | Library (Nanobody, | | PVNSHFMEWYRQAPGKEREWVAAIQH |
| | | | | | | | non-immune) | | SSGFHTYYADSVKGRFTISRDNAKNTVYL |
| | | | | | | | | | QMNSLKPEDTAVYYCNVKDTGTTEDYD |
| | | | | | | | | | YWGQGTQVTVS |
| Sb#32 | Nb | SARS-CoV2 | | | | S; RBD | Phage Display | 1039 | QVQLDESGGGLVQAGGSLRLSCAASGF |
| | | | | | | | Library (Nanobody, | | PVYHAWMEWYRQAPGKEREWVAAITS |
| | | | | | | | non-immune) | | SGRHTYYADSVKGRFTISRDNAKNTVYL |
| | | | | | | | | | QMNSLKPEDTAVYYCNVKDAGRVYNSY |
| | | | | | | | | | DYWGQGTQVTVS |
| Sb#33 | Nb | SARS-CoV2 | | | | S; RBD | Phage Display | 1040 | QVQLVESGGGLVQAGGSLRLSCAASGF |
| | | | | | | | Library (Nanobody, | | PVAHAWMEWYRQAPGKEREWVAAIT |
| | | | | | | | non-immune) | | SYGYKTYYADSVKGRFTISRDNAKNTVYL |
| | | | | | | | | | QMNSLKPEDTAVYYCNVKDTGTYRFYY |
| | | | | | | | | | DYWGQGTQVTVS |
| Sb#34 | Nb | SARS-CoV2 | | | | S; RBD | Phage Display | 1041 | QVQLVESGGGLVQAGGSLRLSCAASGF |
| | | | | | | | Library (Nanobody, | | PVWNQTMVWYRQAPGKEREWVAAI |
| | | | | | | | non-immune) | | WSMGHTYYADSVKGRFTISRDNAKNTV |
| | | | | | | | | | YLQMNSLKPEDTAVYYCNVKDAGVYNR |
| | | | | | | | | | YYDYWGQGTQVTVS |
| Sb#35 | Nb | SARS-CoV2 | | | | S; RBD | Phage Display | 1042 | QVQLVESGGGLVQAGGSLRLSCAASGF |
| | | | | | | | Library (Nanobody, | | PVEHYWMEWYRQAPGKEREWVAAITS |
| | | | | | | | non-immune) | | FGYRTYYADSVKGRFTISRDNAKNTVYL |
| | | | | | | | | | QMNSLKPEDTAVYYCNVKDWGFASHA |
| | | | | | | | | | YDYWGQGIQVTVS |
| Sb#36 | Nb | SARS-CoV2 | | | | S; RBD | Phage Display | 1043 | QVQLVESGGGLVQAGGSLRLSCAASGF |
| | | | | | | | Library (Nanobody, | | PEIAWEMAWYRQAPGKEREWVAAIRS |
| | | | | | | | non-immune) | | FGERTLYADSVKGRFTISRDNAKNTVYL |
| | | | | | | | | | QMNSLKPEDTAVYYCNVKDFGWQHQE |
| | | | | | | | | | YDYWGQGTQVTVS |
| Sb#37 | Nb | SARS-CoV2 | | | | S; RBD | Phage Display | 1044 | QVQLVESGGGLVQAGGSLRLSCAASGF |
| | | | | | | | Library (Nanobody, | | PVYHAYMEWYRQAPGKEREWVAAIYS |
| | | | | | | | non-immune) | | NGEHTYYADSVKGRFTISRDNAKNTVYL |
| | | | | | | | | | QMNSLKPEDTAVYYCNVKDSGSFNQAY |
| | | | | | | | | | DYWGQGTQVTVS |
| Sb#38 | Nb | SARS-CoV2 | | | | S; RBD | Phage Display | 1045 | QVQLVESGGGLVQAGGSLRLSCAASGF |
| | | | | | | | Library (Nanobody, | | PVEWSHMHWYRQAPGKEREWVAAIV |
| | | | | | | | non-immune) | | SKGGYTLYADSVKGRFTISRDNAKNTVYL |
| | | | | | | | | | QMNSLKPEDTAVYYCNVKDYGVHFKRY |
| | | | | | | | | | DYWGQGTQVTVI |
| Sb#39 | Nb | SARS-CoV2 | | | | S; RBD | Phage Display | 1046 | QVQLVESGGGLVQAGGSLRLSCAASGF |
| | | | | | | | Library (Nanobody, | | PVFHVWMEWYRQAPGKEREWVAAID |
| | | | | | | | non-immune) | | SAGWHTYYADSVKGRFTISRDNAKNTV |
| | | | | | | | | | YLQMNSLKPEDTAVYYCNVKDAGNTTS |
| | | | | | | | | | AYDYWGQGTQVTVS |
| Sb#4 | Nb | SARS-CoV2 | | | | S; RBD | Phage Display | 1047 | QVQLVESGGGLVQAGGSLRLSCAASGF |
| | | | | | | | Library (Nanobody, | | PVYAQNMHWYRQAPGKEREWVAAIYS |
| | | | | | | | non-immune) | | HGYWTLYADSVKGRFTISRDNAKNTVYL |
| | | | | | | | | | QMNSLKPEDTAVYYCEVQVGAWYTGQ |
| | | | | | | | | | GTQVTVS |
| Sb#40 | Nb | SARS-CoV2 | | | | S; RBD | Phage Display | 1048 | QVQLVESGGGLVQAGGSLRLSCAASGF |
| | | | | | | | Library (Nanobody, | | PVYYNWMEWYRQAPGKEREWVAAIH |
| | | | | | | | non-immune) | | SNGDETFYADSVKGRFTISRDNAKNTVY |
| | | | | | | | | | LQMNSLKPEDTAVYYCNVKDIDAEAYAY |
| | | | | | | | | | DYWGQGTQVTVS |
| Sb#41 | Nb | SARS-CoV2 | | | | S; RBD | Phage Display | 1049 | QVQLVESGGGLVQAGGSLRLSCAASGF |
| | | | | | | | Library (Nanobody, | | PVYHVWMEWYRQAPGKEREWVAAITS |
| | | | | | | | non-immune) | | SGSHTYYADSVKGRFTISRDNAKNTVYL |
| | | | | | | | | | QMNSLKPEDTAVYYCNVKDSGQWRVQ |
| | | | | | | | | | YDYWGQGTQVTVS |
| Sb#42 | Nb | SARS-CoV2 | | | | S; RBD | Phage Display | 1050 | QVQLVESGGGLVQAGGSLRLSCAASGF |
| | | | | | | | Library (Nanobody, | | PVYWHHMHWYRQAPGKEREWVAAIIS |
| | | | | | | | non-immune) | | WGWYTTYADSVKGRFTISRDNAKNTVY |
| | | | | | | | | | LQMNSLKPEDTAVYYCNVKDHGAQNQ |
| | | | | | | | | | MYDYWGQGTQVTVS |
| Sb#45 | Nb | SARS-CoV2 | | | | S; RBD | Phage Display | 1051 | QVQLVESGGGLVQAGGSLRLSCAASGF |
| | | | | | | | Library (Nanobody, | | PVYRDRMAWYRQAPGKEREWVAAIYS |
| | | | | | | | non-immune) | | AGQQTRYADSVKGRFTISRDNAKNTVYL |
| | | | | | | | | | QMNSLKPEDTAVYYCNVKDVGHHYEYY |
| | | | | | | | | | DYWGQGTQVTVS |
| Sb#46 | Nb | SARS-CoV2 | | | | S; RBD | Phage Display | 1052 | QVQLVESGGGLVQAGGSLRLSCAASGF |
| | | | | | | | Library (Nanobody, | | PVDNGYMHWYRQAPGKEREWVAAID |
| | | | | | | | non-immune) | | SYGWHTIYADSVKGRFTISRDNAKNTVY |
| | | | | | | | | | LQMNSLKPEDTAVYYCNVKDKGQMRA |
| | | | | | | | | | AYDYWGQGTQVTVS |
| Sb#47 | Nb | SARS-CoV2 | | | | S; RBD | Phage Display | 1053 | QVQLVESGGGLVQAGGSLRLSCAASGF |
| | | | | | | | Library (Nanobody, | | PVSWHSMYWYRQAPGKEREWVAAIFS |
| | | | | | | | non-immune) | | EGDWTYYADSVKGRFTISRDNAKNTVYL |
| | | | | | | | | | QMNSLKPEDTAVYYCNVKDYGSSYYKY |
| | | | | | | | | | DYWGQGTQVTVS |
| Sb#48 | Nb | SARS-CoV2 | | | | S; RBD | Phage Display | 1054 | QVQLVESGGGLVQAGGSLRLSCAASGF |
| | | | | | | | Library (Nanobody, | | PVSQSVMAWYRQAPGKEREWVAAIYS |
| | | | | | | | non-immune) | | KGQYTHYADSVKGRFTISRDNAKNTVYL |
| | | | | | | | | | QMNSLKPEDTAVYYCNVKDAGSSYWD |
| | | | | | | | | | YDYWGQGTQVTVS |
| Sb#49 | Nb | SARS-CoV2 | | | | S; RBD | Phage Display | 1055 | QVQLVESGGGSVQAGGSLRLSCAASGSI |
| | | | | | | | Library (Nanobody, | | GQIEYLGWFRQAPGKEREGVAALNTW |
| | | | | | | | non-immune) | | TGRTYYADSVKGRFTVSLDNAKNTVYLQ |
| | | | | | | | | | MNSLKPEDTALYYCAAARWGRTKPLNT |
| | | | | | | | | | YYYSYWGQGTPVTVS |
| Sb#5 | Nb | SARS-CoV2 | | | | S; RBD | Phage Display | 1056 | QVQLVESGGGLVQAGGSLRLSCAASGF |
| | | | | | | | Library (Nanobody, | | PVFSGHMHWYRQAPGKEREWVAAILS |
| | | | | | | | non-immune) | | NGDSTHYADSVKGRFTISRDNAKNTVYL |
| | | | | | | | | | QMNSLKPEDTAVYYCRVHVGAHYFGQ |
| | | | | | | | | | GTQVTVS |
| Sb#50 | Nb | SARS-CoV2 | | | | S; RBD | Phage Display | 1057 | QVQLVESGGGSVQAGGSLRLSCAASGYI |
| | | | | | | | Library (Nanobody, | | DKIVYLGWFRQAPGKEREGVAALYTLSG |
| | | | | | | | non-immune) | | HTYYADSVKGRFTVSLDNAKNTVYLQM |
| | | | | | | | | | NSLKPEDTALYYCAAATEGHAHALYRLH |
| | | | | | | | | | YYWGQGTQVTVS |
| Sb#51 | Nb | SARS-CoV2 | | | | S; RBD | Phage Display | 1058 | QVQLVESGGGLVQAGGSLRLSCAASGF |
| | | | | | | | Library (Nanobody, | | PVYQGEMHWYRQAPGKEREWVAAIRS |
| | | | | | | | non-immune) | | TGVQTWYADSVKGRFTISRDNAKNTVY |
| | | | | | | | | | LQMNSLKPEDTAVYYCRVWVGTHYFG |
| | | | | | | | | | QGTQVTVS |
| Sb#52 | Nb | SARS-CoV2 | | | | S; RBD | Phage Display | 1059 | QVQLVESGGGSVQAGGSLRLSCAASGN |
| | | | | | | | Library (Nanobody, | | IQRIYYLGWFRQAPGKEREGVAALMTYT |
| | | | | | | | non-immune) | | GHTYYADSVKGRFTVSLDNAKNTVYLQ |
| | | | | | | | | | MNSLKPEDTALYYCAAAYVGAENPLPYS |
| | | | | | | | | | MYGYWGQGTQVTVS |
| Sb#53 | Nb | SARS-CoV2 | | | | S; RBD | Phage Display | 1060 | QVQLVESGGGSVQAGGSLRLSCAASGQ |
| | | | | | | | Library (Nanobody, | | ISHIKYLGWFRQAPGKEREGVAALITRW |
| | | | | | | | non-immune) | | GQTYYADSVKGRFTVSLDNAKNTVYLQ |
| | | | | | | | | | MNSLKPEDTALYYCAAADYGASDPLWFI |
| | | | | | | | | | HYLYWGQGTQVTVS |
| Sb#55 | Nb | SARS-CoV2 | | | | S; RBD | Phage Display | 1061 | QVQLVESGGGSVQAGGSLRLSCAASGKI |
| | | | | | | | Library (Nanobody, | | WTIKYLGWFRQAPGKEREGVAALMTR |
| | | | | | | | non-immune) | | WGYTYYADSVKGRFTVSLDNAKNTVYL |
| | | | | | | | | | QMNSLKPEDTALYYCAAANYGSNFPLAE |
| | | | | | | | | | EDYWYWGQGTQVTVS |
| Sb#56 | Nb | SARS-CoV2 | | | | S; RBD | Phage Display | 1062 | QVQLVESGGGSVQAGGSLRLSCAASGN |
| | | | | | | | Library (Nanobody, | | ISQIHYLGWFRQAPGKEREGVAALNTDY |
| | | | | | | | non-immune) | | GYTYYADSVKGRFTVSLDNAKNTVYLQ |
| | | | | | | | | | MNSLKPEDTALYYCAAAYYFGDDIPLW |
| | | | | | | | | | WEAYSYWGQGTQVTVS |
| Sb#58 | Nb | SARS-CoV2 | | | | S; RBD | Phage Display | 1063 | QVQLVESGGGSVQAGGSLRLSCAASGN |
| | | | | | | | Library (Nanobody, | | ISTIEYLGWFRQAPGKEREGVAALYTWH |
| | | | | | | | non-immune) | | GQTYYADSVKGRFTVSLDNAKNTVYLQ |
| | | | | | | | | | MNSLKPEDTALYYCAAARWGRHMPLS |
| | | | | | | | | | ATEYSYWGQGTQVTVS |
| Sb#59 | Nb | SARS-CoV2 | | | | S; RBD | Phage Display | 1064 | QVQLVESGGGSVQAGGSLRLSCAASGN |
| | | | | | | | Library (Nanobody, | | IESIYYLGWFRQAPGKEREGVAALWTG |
| | | | | | | | non-immune) | | DGETYYADSVKGRFTVSLDNAKNTVYLQ |
| | | | | | | | | | MNSLKPEDTALYYCAAAAWGNSAPLTT |
| | | | | | | | | | YRYYYWGQGTQVTVS |
| Sb#6 | Nb | SARS-CoV2 | | | | S; RBD | Phage Display | 1065 | QVQLVESGGGLVQAGGSLRLSCAASGF |
| | | | | | | | Library (Nanobody, | | PVEQGRMYWYRQAPGKEREWVAAIIS |
| | | | | | | | non-immune) | | HGTVTVYADSVKGRFTISRDNAKNTVYL |
| | | | | | | | | | QMNSLKPEDTAVYYCYVYVGAQYWGQ |
| | | | | | | | | | GTQVTVS |
| Sb#61 | Nb | SARS-CoV2 | | | | S; RBD | Phage Display | 1066 | QVQLVESGGGSVQAGGSLRLSCAASGFI |
| | | | | | | | Library (Nanobody, | | YGITYLGWFRQAPGKEREGVAALVTWN |
| | | | | | | | non-immune) | | GQTYYADSVKGRFTVSLDNAKNTVYLQ |
| | | | | | | | | | MNSLKPEDTALYYCAAADWGYDWPLW |
| | | | | | | | | | DEWYWYWGQGTQVTVS |
| Sb#62 | Nb | SARS-CoV2 | | | | S; RBD | Phage Display | 1067 | QVQLVESGGGSVQAGGSLRLSCAASGTI |
| | | | | | | | Library (Nanobody, | | ADIKYLGWFRQAPGKEREGVAALMTR |
| | | | | | | | non-immune) | | WGSTYYADSVKGRFTVSLDNAKNTVYL |
| | | | | | | | | | QMNSLKPEDTALYYCAAANYGANYPLYS |
| | | | | | | | | | QQYSYWGQGTQVTVS |
| Sb#63 | Nb | SARS-CoV2 | | | | S; RBD | Phage Display | 1068 | QVQLVESGGGSVQAGGSLRLSCAASGSI |
| | | | | | | | Library (Nanobody, | | SSIKYLGWFRQAPGKEREGVAALMTRW |
| | | | | | | | non-immune) | | GMTYYADSVKGRFTVSLDNAKNTVYLQ |
| | | | | | | | | | MNSLKPEDTALYYCAAANYGANEPLQY |
| | | | | | | | | | THYNYWGQGTQVTVS |
| Sb#64 | Nb | SARS-CoV2 | | | | S; RBD | Phage Display | 1069 | QVQLVESGGGSVQAGGSLRLSCAASGFI |
| | | | | | | | Library (Nanobody, | | ESIFYLGWFRQAPGKEREGVAALYTYVG |
| | | | | | | | non-immune) | | QTYYADSVKGRFTVSLDNAKNTVYLQM |
| | | | | | | | | | NSLKPEDTALYYCAAASYGAAHPLSIMR |
| | | | | | | | | | YYYWGQGTQVTVS |
| Sb#65 | Nb | SARS-CoV2 | | | | S; RBD | Phage Display | 1070 | QVQLVESGGGSVQAGGSLRLSCAASGTI |
| | | | | | | | Library (Nanobody, | | AHIKYLGWFRQAPGKEREGVAALMTK |
| | | | | | | | non-immune) | | WGQTYYADSVKGRFTVSLDNAKNTVYL |
| | | | | | | | | | QMNSLKPEDTALYYCAAASYGANFPLKA |
| | | | | | | | | | SDYSYWGQGTQVTVS |
| Sb#66 | Nb | SARS-CoV2 | | | | S; RBD | Phage Display | 1071 | QVQLVESGGGSVQAGGSLRLSCAASGSI |
| | | | | | | | Library (Nanobody, | | QAITYLGWFRQAPGKEREGVAALVTW |
| | | | | | | | non-immune) | | NGQTYYADSVKGRFTVSLDNAKNTVYL |
| | | | | | | | | | QMNSLKPEDTALYYCAAADWGYDWPL |
| | | | | | | | | | WDEWYWYWGQGTQVTVS |
| Sb#67 | Nb | SARS-CoV2 | | | | S; RBD | Phage Display | 1072 | QVQLVESGGGSVQAGGSLRLSCAASGSI |
| | | | | | | | Library (Nanobody, | | SSITYLGWFRQAPGKEREGVAALVTYSG |
| | | | | | | | non-immune) | | NTYYADSVKGRFTVSLDNAKNTVYLQM |
| | | | | | | | | | NSLKPEDTALYYCAAATWGHSWPLYND |
| | | | | | | | | | EYWYWGQGSQVTVS |
| Sb#68 | Nb | SARS-CoV2 | | | | S; RBD | Phage Display | 1073 | QVQLVESGGGSVQAGGSLRLSCAASGSI |
| | | | | | | | Library (Nanobody, | | SSITYLGWFRQAPGKEREGVAALITVNG |
| | | | | | | | non-immune) | | HTYYADSVKGRFTVSLDNAKNTVYLQM |
| | | | | | | | | | NSLKPEDTALYYCAAAAWGYAWPLHQ |
| | | | | | | | | | DDYWYWGQGTQVTVS |
| Sb#69 | Nb | SARS-CoV2 | | | | S; RBD | Phage Display | 1074 | QVQLVESGGGSVQAGGSLRLSCAASGSI |
| | | | | | | | Library (Nanobody, | | SSITYLGWFRQAPGKEREGVAALNTFNG |
| | | | | | | | non-immune) | | TTYYADSVKGRFTVSLDNAKNTVYLQM |
| | | | | | | | | | NSLKPEDTALYYCAAATWGYSWPLIAEY |
| | | | | | | | | | NWYWGQGTQVTVS |
| Sb#7 | Nb | SARS-CoV2 | | | | S; RBD | Phage Display | 1075 | QVQLVESGGGLVQAGGSLRLSCAASGF |
| | | | | | | | Library (Nanobody, | | PVLFTYMHWYRQAPGKEREWVAAIWS |
| | | | | | | | non-immune) | | SGNSTWYADSVKGRFTISRDNAKNTVYL |
| | | | | | | | | | QMNSLKPEDTAVYYCFVKVGNWYAGQ |
| | | | | | | | | | GTQVTVS |
| Sb#71 | Nb | SARS-CoV2 | | | | S; RBD | Phage Display | 1076 | QVQLVESGGGSVQAGGSLRLSCAASGSI |
| | | | | | | | Library (Nanobody, | | SSITYLGWFRQAPGKEREGVAALKTQAG |
| | | | | | | | non-immune) | | FTYYADSVKGRFTVSLDNAKNTVYLQM |
| | | | | | | | | | NSLKPEDTALYYCAAANWGYSWPLYEA |
| | | | | | | | | | DDWYWGQGTQVTVS |
| Sb#8 | Nb | SARS-CoV2 | | | | S; RBD | Phage Display | 1077 | QVQLVESGGGLVQAGGSLRLSCAASGF |
| | | | | | | | Library (Nanobody, | | PVNAGNMHWYRQAPGKEREWVAAIQ |
| | | | | | | | non-immune) | | SYGRTTYYADSVKGRFTISRDNAKNTVYL |
| | | | | | | | | | QMNSLKPEDTAVYYCRVFVGMHYFGQ |
| | | | | | | | | | GTQVTVS |
| Sb#9 | Nb | SARS-CoV2 | | | | S; RBD | Phage Display | 1078 | QVQLVESGGGLVQAGGSLRLSCAASGF |
| | | | | | | | Library (Nanobody, | | PVSSSTMTWYRQAPGKEREWVAAINSY |
| | | | | | | | non-immune) | | GWETHYADSVKGRFTISRDNAKNTVYL |
| | | | | | | | | | QMNSLKPEDTAVYYCYVYVGGSYIGQG |
| | | | | | | | | | TQVTVS |
| Sb100 | Nb | SARS-CoV2 | | SARS-CoV2 | | S; RBD | Phage Display | 1079 | QVQLVESGGGSVQAGGSLRLSCAASGSI |
| | | | | | | | Library (Nanobody, | | SSITYLGWFRQAPGKEREGVAALVTSDG |
| | | | | | | | non-immune) | | RTYYADSVKGRFTVSLDNAKNTVYLQM |
| | | | | | | | | | NSLKPEDTALYYCAAANWGYSWPLYQT |
| | | | | | | | | | EYWYWGQGTQVTVSS |
| Sb12 | Nb | SARS-CoV2 | | SARS-CoV2 | | S; RBD | Phage Display | 1080 | QVQLVESGGGLVQAGGSLRLSCAASGF |
| | | | | | | | Library (Nanobody, | | PVQLYWMEWYRQAPGKEREWVAAITS |
| | | | | | | | non-immune) | | DGDYTEYADSVKGRFTISRDNAKNTVYL |
| | | | | | | | | | QMNSLKPEDTAVYYCYVKVGEWYYGQ |
| | | | | | | | | | GTQVTVSS |
| Sb13 | Nb | SARS-CoV2 | | | | S; RBD | Phage Display | 1081 | QVQLVESGGGLVQAGGSLRLSCAASGF |
| | | (weak) | | | | | Library (Nanobody, | | PVENYYMRWYRQAPGKEREWVAAIES |
| | | | | | | | non-immune) | | SGAETRYADSVKGRFTISRDNAKNTVYL |
| | | | | | | | | | QMNSLKPEDTAVYYCYVVVGWGYAGQ |
| | | | | | | | | | GTQVTVSS |
| Sb15 | Nb | SARS-CoV2 | | | | S; RBD | Phage Display | 1082 | QVQLVESGGGLVQAGGSLRLSCAASGF |
| | | | | | | | Library (Nanobody, | | PVYEHYMRWYRQAPGKEREWVAAIQS |
| | | | | | | | non-immune) | | HGNHTAYADSVKGRFTISRDNAKNTVYL |
| | | | | | | | | | QMNSLKPEDTAVYYCFVVVGNGYTGQ |
| | | | | | | | | | GTQVTVSS |
| Sb16 | Nb | SARS-CoV2 | | | | S; RBD | Phage Display | 1083 | QVQLVESGGGLVRAGGSLRLSCAASGF |
| | | | | | | | Library (Nanobody, | | PVASQEMTWYRQAPGKEREWVAAISSS |
| | | | | | | | non-immune) | | GRQTEYADSVKGRFTISRDNAKNTVYLQ |
| | | | | | | | | | MNSLKPEDTAVYYCYVYVGGSYIGQGT |
| | | | | | | | | | QVTVSS |
| Sb17 | Nb | SARS-CoV2 | | SARS-CoV2 | | S; RBD | Phage Display | 1084 | QVQLVESGGGLVQAGGSLRLSCAASGF |
| | | | | | | | Library (Nanobody, | | PVKASEMEWYRQAPGKEREWVAAIASI |
| | | | | | | | non-immune) | | GYNTYYADSVKGRFTISRDNAKNTVYLQ |
| | | | | | | | | | MNSLKPEDTAVYYCLVYVGATYIGQGTQ |
| | | | | | | | | | VTVSS |
| Sb2 | Nb | SARS-CoV2 | | | | S; RBD | Phage Display | 1085 | QVQLVESGGGLVQAGGSLRLSCAASGF |
| | | | | | | | Library (Nanobody, | | PVSNEEMTWYRQAPGKEREWVAAIAS |
| | | | | | | | non-immune) | | NGNQTEYADSVKGRFTISRDNAKNTVYL |
| | | | | | | | | | QMNSLKPEDTAVYYCYVYVGASYIGQG |
| | | | | | | | | | TQVTVSS |
| Sb21 | Nb | SARS-CoV2 | | | | S; RBD | Phage Display | 1086 | QVQLVESGGGLVQAGGSLRLSCAASGF |
| | | | | | | | Library (Nanobody, | | PVKESEMTWYRQARGKEREWVAAINS |
| | | | | | | | non-immune) | | HGMTTHYADSVKGRFTISRDNAKNTVY |
| | | | | | | | | | LQMNSLKPEDTAVYYCYVYVGGSYIGQ |
| | | | | | | | | | GTQVTVSS |
| Sb22 | Nb | SARS-CoV2 | | SARS-CoV2 | | S; RBD | Phage Display | 1087 | QVQLVESGGGLVQAGGSLRLSCAASGF |
| | | | | | | | Library (Nanobody, | | PVNHYEMEWYRQAPGREREWVAAIM |
| | | | | | | | non-immune) | | DSTGYETAYADSVKGRFTISRDNAKNTV |
| | | | | | | | | | YLQMNSLKPEDTAVYYCYVYVGASYIGQ |
| | | | | | | | | | GTQVTVSS |
| Sb23 | Nb | SARS-CoV2 | | SARS-CoV2 | | S; RBD | Phage Display | 1088 | QVQLVESGGGLVQAGGSLRLSCAASGF |
| | | | | | | | Library (Nanobody, | | PVESENMHWYRQAPGKEREWVAAIYS |
| | | | | | | | non-immune) | | TGGWTLYADSVKGRFTISRDNAKNTVYL |
| | | | | | | | | | QMNSLKPEDTAVYYCAVQVGYWYEGQ |
| | | | | | | | | | GTQVTVSS |
| Sb25 | Nb | SARS-CoV2 | | | | S; RBD | Phage Display | 1089 | QVQLVESGGGLVQAGGSLRLSCAASGF |
| | | | | | | | Library (Nanobody, | | PVESTEMTWYRQAPGKEREWVAAIESE |
| | | | | | | | non-immune) | | GHGTEYADSVKGRFTISRDNAKNTVYLQ |
| | | | | | | | | | MNSLKPEDTAVYYCYVYVGAGYIGQGT |
| | | | | | | | | | QVTVSS |
| Sb27 | Nb | SARS-CoV2 | | | | S; RBD | Phage Display | 1090 | QVQLVESGGGLVQAGGSLRLSCAASGF |
| | | | | | | | Library (Nanobody, | | PVKASEMVWYRQAPGKEREWVAAILS |
| | | | | | | | non-immune) | | QGHATEYADSVKGRFTISRDNAKNTVYL |
| | | | | | | | | | QMNSLKPEDTAVYYCYVWVGRSYIGQG |
| | | | | | | | | | TQVTVSS |
| Sb28 | Nb | SARS-CoV2 | | | | S; RBD | Phage Display | 1091 | QVQLVESGGGLVQAGGSLRLSCAASGF |
| | | | | | | | Library (Nanobody, | | PVYSAEMEWYRQAPGKEREWVAAISSY |
| | | | | | | | non-immune) | | GTNTYYADSVKGRFTISRDNAKNTVYLQ |
| | | | | | | | | | MNSLKPEDTAVYYCYVYVGSSYIGQGTQ |
| | | | | | | | | | VTVSS |
| Sb30 | Nb | SARS-CoV2 | | | | S; RBD | Phage Display | 1092 | QVQLVESGGGLVQAGGSLRLSCAASGF |
| | | | | | | | Library (Nanobody, | | PVWYKEMEWYRQAPGKEREWVAAITS |
| | | | | | | | non-immune) | | AGHHTYYADSVKGRFTISRDNAKNTVYL |
| | | | | | | | | | QMNSLKPEDTAVYYCYVYVGESYIGQGT |
| | | | | | | | | | QVTVSS |
| Sb32 | Nb | SARS-CoV2 | | | | S; RBD | Phage Display | 1093 | QVQLVESGGGLVQAGGSLRLSCAASGF |
| | | | | | | | Library (Nanobody, | | PVAHKSMWWYRQAPGKEREWVAAIES |
| | | | | | | | non-immune) | | TGDTTRYADSVKGRFTISRDNAKNTVYL |
| | | | | | | | | | QMNSLKPEDTAVYYCVVWVGEVYRGQ |
| | | | | | | | | | GTQVTVSS |
| Sb37 | Nb | SARS-CoV2 | | | | S; RBD | Phage Display | 1094 | QVQLVESGGGLVQAGGSLRLSCAASGF |
| | | (weak) | | | | | Library (Nanobody, | | PVYNTWMEWYRQAPGKEREWVAAITS |
| | | | | | | | non-immune) | | YGFHTYYADSVKGRFTISRDNAKNTVYL |
| | | | | | | | | | QMNSLKPEDTAVYYCNVKDEGNTTAYY |
| | | | | | | | | | DYWGQGTQVTVSS |
| Sb38 | Nb | SARS-CoV2 | | | | S; RBD | Phage Display | 1095 | QVQLVESGGGLVQAGGSLRLSCAASGF |
| | | | | | | | Library (Nanobody, | | PVYWAHMTWYRQAPGKEREWVAAIV |
| | | | | | | | non-immune) | | SSGAYTAYADSVKGRFTISRDNAKNTVYL |
| | | | | | | | | | QMNSLKPEDTAVYYCNVKDFGTQEHYY |
| | | | | | | | | | DYWGQGTQVTVSS |
| Sb39 | Nb | SARS-CoV2 | | | | S; RBD | Phage Display | 1096 | QVQLVESGGGPVQAGGSLRLSCAASGF |
| | | | | | | | Library (Nanobody, | | PVMWSHMHWYRQAPGKEREWVAAIV |
| | | | | | | | non-immune) | | SYGAYTIYADSVKGRFTISRDNAKNTVYL |
| | | | | | | | | | QMNSLKPEDTAVYYCNVKDFGGYRYYY |
| | | | | | | | | | DYWGQGTQVTVSS |
| Sb40 | Nb | SARS-CoV2 | | | | S; RBD | Phage Display | 1097 | QVQLVESGGGLVQAGGSLRLSCAASGF |
| | | | | | | | Library (Nanobody, | | PVQGTWMEWYRQAPGKEREWVAAIT |
| | | | | | | | non-immune) | | SVGYRTYYADSVKGRFTISRDNAKNTVYL |
| | | | | | | | | | QMNSLKPEDTAVYYCNVKDEGAIAKNY |
| | | | | | | | | | DYWGQGTQVTVSS |
| Sb42 | Nb | SARS-CoV2 | | SARS-CoV2 | | S; RBD | Phage Display | 1098 | QVQLVESGGGLVQAGGSLRLSCAASGF |
| | | | | | | | Library (Nanobody, | | PVYNTWMEWYRQAPGKEREWVAAITS |
| | | | | | | | non-immune) | | WGFKTYYADSVKGRFTISRDNAKNTVYL |
| | | | | | | | | | QMNSLKPEDTAVYYCNVKDEGYTGYYY |
| | | | | | | | | | DYWGQGTQVTVSS |
| Sb43 | Nb | SARS-CoV2 | | SARS-CoV2 | | S; RBD | Phage Display | 1099 | QVQLVESGGGLVQAGGSLRLSCAASGF |
| | | | | | | | Library (Nanobody, | | PVEWTHMHWYRQAPGKEREWVAAIA |
| | | | | | | | non-immune) | | SSGAYTVYADSVKGRFTISRDNAKNTVYL |
| | | | | | | | | | QMNSLKPEDTAVYYCNVKDWGSQDRY |
| | | | | | | | | | YDYWGQGTQVTVSS |
| Sb45 | Nb | SARS-CoV2 | | | | S; RBD | Phage Display | 1100 | QVQLVESGGGLVQAGGSLRLSCAASGF |
| | | | | | | | Library (Nanobody, | | PVAGTWMEWYRQAPGKEREWVAAITS |
| | | | | | | | non-immune) | | YGYRTYYADSVKGRFTISRDNAKNTVYL |
| | | | | | | | | | QMNSLKPEDTAVYYCNVKDEGKSSQVY |
| | | | | | | | | | DYWGQGTQVTVSS |
| Sb46 | Nb | SARS-CoV2 | | SARS-CoV2 | | S; RBD | Phage Display | 1101 | QVQLVESGGGLVQAGGSLRLSCAASGF |
| | | | | | | | Library (Nanobody, | | PVNYTIMFWYRKAPGKEREWVAAIKSH |
| | | | | | | | non-immune) | | GATTLYADSVKGRFTISRDNAKNTVYLQ |
| | | | | | | | | | MNSLKPEDTAVYYCNVKDVGNDQKSYD |
| | | | | | | | | | YWGQGTQVTVSS |
| Sb47 | Nb | SARS-CoV2 | | | | S; RBD | Phage Display | 1102 | QVQLVESGGGLVQAGGSLRLSCAASGF |
| | | | | | | | Library (Nanobody, | | PVVWAHMHWYRQAPGKEREWVAAIT |
| | | | | | | | non-immune) | | SEGAHTIYADSVKGRFTISRDNAKNTVYL |
| | | | | | | | | | QMNSLKPEDTAVYYCNVKDWGTYSTYY |
| | | | | | | | | | DYWGQGTQVTVSS |
| Sb5 | Nb | SARS-CoV2 | | | | S; RBD | Phage Display | 1103 | QVQLVESGGGLVQAGGSLRLSCAASGF |
| | | (weak) | | | | | Library (Nanobody, | | PVAQQEMTWYRQAPGKEREWVAAISS |
| | | | | | | | non-immune) | | IGSITHYADSVKGRFTISRDNAKNTVYLQ |
| | | | | | | | | | MNSLKPEDTAVYYCYVYVGASYIGQGT |
| | | | | | | | | | QVTVSS |
| Sb50 | Nb | SARS-CoV2 | | | | S; RBD | Phage Display | 1104 | QVQLVESGGGLVQAGGSLRLSCAASGF |
| | | | | | | | Library (Nanobody, | | PVQYEHMHWYRQAPGKEREWVAAIVS |
| | | | | | | | non-immune) | | EGAYTHYADSVKGRFTISRDNAKNTVYL |
| | | | | | | | | | QMNSLKPEDTAVYYCNVKDWGWLAQY |
| | | | | | | | | | YDYWGQGTQVTVSS |
| Sb52 | Nb | SARS-CoV2 | | | | S; RBD | Phage Display | 1105 | QVQLVESGGGLVQAGGSLRLSCAASGF |
| | | | | | | | Library (Nanobody, | | PVNYNWMEWYRQAPGKEREWVAAIT |
| | | | | | | | non-immune) | | SWGYKTYYADSVKGRFTISRDNAKNTVY |
| | | | | | | | | | LQMNSLKPEDTAVYYCNVKDEGMWQ |
| | | | | | | | | | HYYDYWGQGTQVTVSS |
| Sb54 | Nb | SARS-CoV2 | | | | S; RBD | Phage Display | 1106 | QVQLVESGGGLVQAGGSLRLSCAASGF |
| | | | | | | | Library (Nanobody, | | PVWNTWMEWYRQAPGKEREWVAAIT |
| | | | | | | | non-immune) | | SYGFKTYYADSVKGRFTISRDNAKNTVYL |
| | | | | | | | | | QMNSLKPEDTAVYYCNVKDEGNSQSHY |
| | | | | | | | | | DYWGQGTQVTVSS |
| Sb56 | Nb | SARS-CoV2 | | | | S; RBD | Phage Display | 1107 | QVQLVESGGGLVQAGGSLRLSCAASGF |
| | | | | | | | Library (Nanobody, | | PVEMWSMEWYRQAPGKEREWVAAI |
| | | | | | | | non-immune) | | MSFGYQTWYADSVKGRFTISRDNAKNT |
| | | | | | | | | | VYLQMNSLKPEDTAVYYCNVKDAGNSK |
| | | | | | | | | | ALYDYWGQGTQVTVSS |
| Sb57 | Nb | SARS-CoV2 | | | | S; RBD | Phage Display | 1108 | QVQLVESGGGLVQAGGSLRLSCAASGF |
| | | | | | | | Library (Nanobody, | | PVEHDHMHWYRQAPGKEREWVAAIVS |
| | | | | | | | non-immune) | | QGAYTVYADSVKGRFTISRDNAKNTVYL |
| | | | | | | | | | QMNSLKPEDTAVYYCNVKDWGRAGAR |
| | | | | | | | | | YDYWGQGTQVTVSS |
| Sb58 | Nb | SARS-CoV2 | | | | S; RBD | Phage Display | 1109 | QVQLVESGGGLVQAGGSLRLSCAASGF |
| | | (weak) | | | | | Library (Nanobody, | | PVDAAWMEWYRQAPGKEREWVAAIT |
| | | | | | | | non-immune) | | SYGYRTYYADSVKGRFTISRDNAKNTVYL |
| | | | | | | | | | QMNSLKPEDTAVYYCNVKDMDRWRTT |
| | | | | | | | | | YDYWGQGTQVTVSS |
| Sb6 | Nb | SARS-CoV2 | | | | S; RBD | Phage Display | 1110 | QVQLVESGGGLVQAGGSLRLSCAASGF |
| | | | | | | | Library (Nanobody, | | PVNHTRMYWYRQAPGKEREWVAAIQS |
| | | | | | | | non-immune) | | HGQNTFYADSVKGRFTISRDNAKNTVYL |
| | | | | | | | | | QMNSLKPEDTAVYYCYVWVGNQYWG |
| | | | | | | | | | QGTQVTVSS |
| Sb60 | Nb | SARS-CoV2 | | | | S; RBD | Phage Display | 1111 | QVQLVESGGGLVQAGGSLRLSCAASGF |
| | | | | | | | Library (Nanobody, | | PVVAWQMTWYRQAPGKEREWVAAIR |
| | | | | | | | non-immune) | | SFGVSTHYADSVKGRFTISRDNAKNTVYL |
| | | | | | | | | | QMNSLKPEDTAVYYCNVKDWGYEYEG |
| | | | | | | | | | YDYWGQGTQVTVSS |
| Sb61 | Nb | SARS-CoV2 | | | | S; RBD | Phage Display | 1112 | QVQLVESGGGLVQAGGSLRLSCAASGF |
| | | | | | | | Library (Nanobody, | | PVQHEWMEWYRQAPGKEREWVAAIT |
| | | | | | | | non-immune) | | SYGYRTYYADSVKGRFTISRDNAKNTVYL |
| | | | | | | | | | QMNSLKPEDTAVYYCNVKDTGTYQAW |
| | | | | | | | | | YDYWGQGTQVTVSS |
| Sb62 | Nb | SARS-CoV2 | | SARS-CoV2 | | S; RBD | Phage Display | 1113 | QVQLVESGGGLVQAGGSLRLSCAASGF |
| | | | | | | | Library (Nanobody, | | PVEQEHMYWYRQASGKEREWVAAIVS |
| | | | | | | | non-immune) | | EGAYTAYADSVKGRFTISRDNAKNTVYL |
| | | | | | | | | | QMNSLKPEDTAVYYCNVKDWGGYQW |
| | | | | | | | | | YYDYWGQGTQVTVSS |
| Sb63 | Nb | SARS-CoV2 | | | | S; RBD | Phage Display | 1114 | QVQLVESGGGLVQAGGSLRLSCAASGF |
| | | | | | | | Library (Nanobody, | | PVNAEIMFWYRQAPGKEREWVAAIKSA |
| | | | | | | | non-immune) | | GTTTLYADSVKGRFTISRDNAKNTVYLQ |
| | | | | | | | | | MNSLKPEDTAVYYCNVKDYGAQAHYYD |
| | | | | | | | | | YWGQGTQVTVSS |
| Sb67 | Nb | SARS-CoV2 | | | | S; RBD | Phage Display | 1115 | QVQLVESGGGLVQAGGSLRLSCAASGF |
| | | | | | | | Library (Nanobody, | | PVMWAHMAWYRQAPGKEREWVAAI |
| | | | | | | | non-immune) | | VSAGAYTHYADSVKGRFTISRDNAKNTV |
| | | | | | | | | | YLQMNSLKPEDTAVYYCNVKDWGTYNS |
| | | | | | | | | | YYDYWGQGTQVTVSS |
| Sb7 | Nb | SARS-CoV2 | | SARS-CoV2 | | S; RBD | Phage Display | 1116 | QVQLVESGGGLVQAGGSLRLSCAASGF |
| | | | | | | | Library (Nanobody, | | PVNAEEMEWYRQAPGKEREWVAAISS |
| | | | | | | | non-immune) | | SGDWTYYADSVKGRFTISRDNAKNTVYL |
| | | | | | | | | | QMNSLKPEDTAVYYCLVYVGSTYIGQGT |
| | | | | | | | | | QVTVSS |
| Sb71 | Nb | SARS-CoV2 | | | | S; RBD | Phage Display | 1117 | QVQLVESGGGSVQAGGSLRLSCAASGN |
| | | | | | | | Library (Nanobody, | | IQHIKYLGWFRQAPGREREGVAALMTR |
| | | | | | | | non-immune) | | YGQTYYADSVKGRFTVSLDNAKNTVYLQ |
| | | | | | | | | | MNSLKPEDTALYYCAAAHYGDNFPLAY |
| | | | | | | | | | QAYLYWGQGTQVTVSS |
| Sb75 | Nb | SARS-CoV2 | | | | S; RBD | Phage Display | 1118 | QVQLVESGGGSVQAGGSLRLSCAASGYI |
| | | | | | | | Library (Nanobody, | | NQIYYLGWFRQAPGKEREGVAALNTYQ |
| | | | | | | | non-immune) | | GQTYYADSVKGRFTVSLDNAKNTVYLQ |
| | | | | | | | | | MNSLKPEDTALYYCAAARWGRDEPLYH |
| | | | | | | | | | YYYSYWGQGTQVTVSS |
| Sb76 | Nb | SARS-CoV2 | | SARS-CoV2 | | S; RBD | Phage Display | 1119 | QVQLVESGGGLVQAGGSLRLSCAASSFP |
| | | | | | | | Library (Nanobody, | | VDTYHMAWYRQAPGKEREWVAAIVS |
| | | | | | | | non-immune) | | WGWRTYYADSVKGRFTISRDNAKNTVY |
| | | | | | | | | | LQMNSLKPEDTAVYYCNVKDIGAQEVH |
| | | | | | | | | | YDYWGQGTQVTVSS |
| Sb78 | Nb | SARS-CoV2 | | | | S; RBD | Phage Display | 1120 | QVQLVESGGGSVQAGGSLRLSCAASGYI |
| | | | | | | | Library (Nanobody, | | KSIKYLGWFRQAPGKEREGVAALMTRY |
| | | | | | | | non-immune) | | GETYYADSVKGRFTVSLDNAKNTVYLQ |
| | | | | | | | | | MNSLKPEDTALYYCAAANYGNNWPLTG |
| | | | | | | | | | VNYWYWGQGTQVTVSS |
| Sb8 | Nb | SARS-CoV2 | | | | S; RBD | Phage Display | 1121 | QVQLVESGGGLVQAGGSLRLSCAASGF |
| | | | | | | | Library (Nanobody, | | PVKSYEMEWYRQAPGKEREWVAAISSY |
| | | | | | | | non-immune) | | GEYTEYADSVKGRFTISRDNAKNTVYLQ |
| | | | | | | | | | MNSLKPEDTAVYYCYVWVGDSYLGQGT |
| | | | | | | | | | QVTVSS |
| Sb83 | Nb | SARS-CoV2 | | | | S; RBD | Phage Display | 1122 | QVQLVESGGGSVQAGGSLRLSCAASGG |
| | | | | | | | Library (Nanobody, | | ITHIVYLGWFRQAPGKEREGVAALMTR |
| | | | | | | | non-immune) | | WGTTYYADSVKGRFTVSLDNAKNTVYL |
| | | | | | | | | | QMNSLKPEDTALYYCAAAKYGQNFPLSY |
| | | | | | | | | | HAYRYWGQGTQVTVSS |
| Sb84 | Nb | SARS-CoV2 | | | | S; RBD | Phage Display | 1123 | QVQLVESGGGSVQAGGSLRLSCAASGYI |
| | | | | | | | Library (Nanobody, | | KHIEYLGWFRQAPGKEREGVAALKTSSG |
| | | | | | | | non-immune) | | STYYADSVKGRFTVSLDNAKNTVYLQM |
| | | | | | | | | | NSLKPEDTALYYCAAARYGRSDPLHYHE |
| | | | | | | | | | YSYWGQGTQVTVSS |
| Sb85 | Nb | SARS-CoV2 | | | | S; RBD | Phage Display | 1124 | QVQLVESGGGSVQAGGSLRLSCAASGSI |
| | | | | | | | Library (Nanobody, | | SSITYLGWFRQAPGKEREGVAALVTSRG |
| | | | | | | | non-immune) | | KTYYADSVKGRFTVSLDNAKNTVYLQM |
| | | | | | | | | | NSLKPEDTALYYCAAASWGYTWPLYTYD |
| | | | | | | | | | YWYWGQGTQVTVSS |
| Sb88 | Nb | SARS-CoV2 | | | | S; RBD | Phage Display | 1125 | QVQLVESGGGLVQAGGSLRLSCAASGF |
| | | | | | | | Library (Nanobody, | | PVWAHHMLWYRQAPGKEREWVAAIA |
| | | | | | | | non-immune) | | SWGANTAYADSVKGRFTISRDNAKNTV |
| | | | | | | | | | YLQMNSLKPEDTAVYYCNVKDSGQYRE |
| | | | | | | | | | NYDYWGQGTQVTVSS |
| Sb9 | Nb | SARS-CoV2 | | | | S; RBD | Phage Display | 1126 | QVQLVESGGGLVQAGGSLRLSCAASGF |
| | | | | | | | Library (Nanobody, | | PVGQQHMYWYRQAPGKEREWVAAIYS |
| | | | | | | | non-immune) | | YGHITKYADSVKGRFTISRDNAKNTVYLQ |
| | | | | | | | | | MNSLKPEDTAVYYCWVYVGDYYEGQG |
| | | | | | | | | | TQVTVSS |
| Sb90 | Nb | SARS-CoV2 | | | | S; RBD | Phage Display | 1127 | QVQLVESGGGSVQAGGSLRLSCAASGA |
| | | | | | | | Library (Nanobody, | | INQIYYLGWFRQAPGKEREGVAALSTKY |
| | | | | | | | non-immune) | | GETYYADSVKGRFTVSLDNAKNTVYLQ |
| | | | | | | | | | MNSLKPEDTALYYCAAARWGRQYPLTF |
| | | | | | | | | | VYYSYWGQGTQVTVSS |
| Sb93 | Nb | SARS-CoV2 | | | | S; RBD | Phage Display | 1128 | QVQLVESGGGSVQAGGSLRLSCAASGH |
| | | | | | | | Library (Nanobody, | | IAQIEYLGWFRQAPGKEREGVAALSTNQ |
| | | | | | | | non-immune) | | GYTYYADSVKGRFTVSLDNAKNTVYLQ |
| | | | | | | | | | MNSLKPEDTALYYCAAARWGRTYPLSY |
| | | | | | | | | | MAYTYWGQGTQVTVSS |
| Sb94 | Nb | SARS-CoV2 | | | | S; RBD | Phage Display | 1129 | QVQLVESGGGSVQAGGSLRLSCAASGYI |
| | | | | | | | Library (Nanobody, | | TMIEYLGWFRQAPGKEREGVAALNTHT |
| | | | | | | | non-immune) | | GGTYYADSVKGRFTVSLDNAKNTVYLQ |
| | | | | | | | | | MNSLKPEDTALYYCAAARWGRYEPLHY |
| | | | | | | | | | AYYSYWGQGTQVTVSS |
| Sb95 | Nb | SARS-CoV2 | | | | S; RBD | Phage Display | 1130 | QVQLVESGGGSVQAGGSLRLSCAASGN |
| | | | | | | | Library (Nanobody, | | IYNIKYLGWFRQAPGKEREGVAALMTRY |
| | | | | | | | non-immune) | | GETYYADSVKGRFTVSLDNAKNTVYLQ |
| | | | | | | | | | MNSLKPEDTALYYCAAASYGANWPLVS |
| | | | | | | | | | AAYTYWGQGTQVTVSS |
| Sb97 | Nb | SARS-CoV2 | | | | S; RBD | Phage Display | 1131 | QVQLVESGGGSVQAGGSLRLSCAASGA |
| | | | | | | | Library (Nanobody, | | ISTIEYLGWFRQAPGREREGVAALYTER |
| | | | | | | | non-immune) | | GYTYYADSVKGRFTVSLDNAKNTVYLQ |
| | | | | | | | | | MNSLKPEDTALYYCAAARYGHAQAPLH |
| | | | | | | | | | YFWYGYWGQGTQVTVSS |
| SR1 | Nb | SARS-CoV2 | | | | S; RBD | Phage Display | 1132 | QVQLVESGGGLVQAGGSLRLSCAASGF |
| | | | | | | | Library (Nanobody, | | PVAAYEMEWYRQAPGKEREWVAAINS |
| | | | | | | | non-immune) | | MGDQTYYADSVKGRFTISRDNAKNTVY |
| | | | | | | | | | LQMNSLKPEDTAVYYCYVYVGFSYIGQG |
| | | | | | | | | | TQVTVSS |
| SR13 | Nb | SARS-CoV2 | | | | S; RBD | Phage Display | 1133 | QVQLVESGGGLVQAGGSLRLSCAASGF |
| | | | | | | | Library (Nanobody, | | PVDYMEMEWFRQAPGKEREWVAAITS |
| | | | | | | | non-immune) | | NGRETYYADSVKGRFTVSRDNAKNTVYL |
| | | | | | | | | | QMNSLKPEDTAVYYCYVYVGSSYIGQGT |
| | | | | | | | | | QVTVSS |
| SR18 | Nb | SARS-CoV2 | | | | S; RBD | Phage Display | 1134 | QVQLVESGGGLVQAGGSLRLSCAASGF |
| | | | | | | | Library (Nanobody, | | PVWFQEMEWYRQAPGKEREWVAAISS |
| | | | | | | | non-immune) | | QGTHTYYADSVKGRFTISRDNAKNTVYL |
| | | | | | | | | | QMNSLKPEDTAVYYCYVYVGASYLGQG |
| | | | | | | | | | TQVTVSS |
| SR31 | Nb | SARS-CoV2 | | | | S; RBD | Phage Display | 1135 | QVQLVESGGGLVQAGGSLRLSCAASGF |
| | | | | | | | Library (Nanobody, | | PVWQGEMAWYRQAPGKEREWVAAIS |
| | | | | | | | non-immune) | | SMGYKTYYADSVKGRFTISRDNAKNTVY |
| | | | | | | | | | LQMNSLKPEDTAVYYCAVMVGFWYAG |
| | | | | | | | | | QGTQVTVSS |
| SR34 | Nb | SARS-CoV2 | | | | S; RBD | Phage Display | 1136 | QVQLVESGGGLVQAGGSLRLSCAAGGF |
| | | | | | | | Library (Nanobody, | | PVKDHEMEWYRQAPGKEREWVAAITS |
| | | | | | | | non-immune) | | SGWGTNTYYADSVKGRFTISRDNAKNT |
| | | | | | | | | | VYLQMNSLKPEDTAVYYCYVYVGSSYIG |
| | | | | | | | | | QGTQVTVSS |
| SR38 | Nb | SARS-CoV2 | | | | S; RBD | Phage Display | 1137 | QVQLVESGGGLVQAGGSLRLSCAASGF |
| | | | | | | | Library (Nanobody, | | PVNQEEMEWYRQAPGKEREWVAAIKS |
| | | | | | | | non-immune) | | WGTLTAYADSVKGRFTISRDNAKNTVYL |
| | | | | | | | | | QMNSLKPEDTAVYYCAVHVGQTYIGQG |
| | | | | | | | | | TQVTVSS |
| SR4 | Nb | SARS-CoV2 | | | | S; RBD | Phage Display | 1138 | QVQLVESGGGLVQAGGSLRLSCAASGF |
| | | | | | | | Library (Nanobody, | | PVYSWNMWWYRQAPGKEREWVAAIE |
| | | | | | | | non-immune) | | SHGDSTRYADSVKGRFTISRDNAKNTVY |
| | | | | | | | | | LQMNSLKPEDTAVYYCYVWVGHTYYG |
| | | | | | | | | | QGTQVTVSS |
| SR5 | Nb | SARS-CoV2 | | | | S; RBD | Phage Display | 1139 | QVQLVESGGGLVQAGGSLRLRCAASGF |
| | | | | | | | Library (Nanobody, | | PVETTEMEWYRQAPGKEREWVAAISSY |
| | | | | | | | non-immune) | | GSETYYADSVKGRFTISRDNAKNTVYLQ |
| | | | | | | | | | MNSLKPEDTAVYYCYVYVGTSYLGQGT |
| | | | | | | | | | QVTVSS |
| SR7 | Nb | SARS-CoV2 | | | | S; RBD | Phage Display | 1140 | QVQLVESGGGLVQAGGSLRLSCAASGF |
| | | | | | | | Library (Nanobody, | | PVWSNEMEWYRQAPGKEREWVAAITS |
| | | | | | | | non-immune) | | YGTTEYADSVKGRFTISRDNAKNTVYLQ |
| | | | | | | | | | MNSLKPEDTAVYYCYVYVGYSYIGQGTQ |
| | | | | | | | | | VTVSS |
| Ty1 | Nb | SARS-CoV2 | | SARS-CoV2 | | S; RBD | Immunised Alpaca | 1141 | QVQLVETGGGLVQPGGSLRLSCAASGF |
| | | | | | | | | | TFSSVYMNWVRQAPGKGPEWVSRISP |
| | | | | | | | | | NSGNIGYTDSVKGRFTISRDNAKNTLYL |
| | | | | | | | | | QMNNLKPEDTALYYCAIGLNLSSSSVRG |
| | | | | | | | | | QGTQVTVSS |
| VH-Fc- | Nb | SARS-CoV2 | | SARS-CoV2 | | S; RBD | Phage Display | | ND |
| B01 | | | | | | | (Human) | | |
| VH2-A01- | Nb | SARS-CoV2 | | SARS-CoV2 | | S; RBD | Phage Display | | ND |
| B01 | | | | | | | (Human) | | |
| VH2-A01- | Nb | SARS-CoV2 | | SARS-CoV2 | | S; RBD | Phage Display | | ND |
| B02 | | | | | | | (Human) | | |
| VH3-B01 | Nb | SARS-CoV2 | | SARS-CoV2 | | S; RBD | Phage Display | | ND |
| | | | | | | | (Human) | | |
| VHH-72 | Nb | SARS-CoV1, | | SARS-CoV2 | | S; RBD | Immunised Llama | 1142 | QVQLQESGGGLVQAGGSLRLSCAASGR |
| | | SARS-CoV2 | | and SARS- | | | | | TFSEYAMGWFRQAPGKEREFVATISWS |
| | | | | CoV1 | | | | | GGSTYYTDSVKGRFTISRDNAKNTVYLQ |
| | | | | | | | | | MNSLKPDDTAVYYCAAAGLGTVVSEW |
| | | | | | | | | | DYDYDYWGQGTQVTVSS |
| W23UACh | Nb | SARS-CoV2 | | | | S; RBD | Immunised Alpaca | 1143 | QVQLVESGGGLVQPGESLRLSCAASGNI |
| | | | | | | | | | FGIAAVHWFRKAPGKEREFTAGFGSDG |
| | | | | | | | | | STNYANSVKGRFTISRDNAKNTTYLQMN |
| | | | | | | | | | SLKPEDTAVYYCHALIKNELGFLDYWGP |
| | | | | | | | | | GTQVTVSS |
| W25UACh | Nb | SARS-CoV2 | | | | S; RBD | Immunised Alpaca | 1144 | QVQLVESGGGLVQPGESLRLSCAASGSI |
| | | | | | | | | | FGIYAVHWFRMAPGKEREFTAGFGSHG |
| | | | | | | | | | STNYAASVKGRFTMSRDNAKNTTYLQM |
| | | | | | | | | | NSLKPADTAVYYCHALIKNELGFLDYWG |
| | | | | | | | | | PGTQVTVSS |
| | | SEQ | | | | | | SEQ | |
| | | ID | | Heavy V | Heavy J | Light V | Light J | ID | |
| | Name | NO.: | VL | Gene | Gene | Gene | Gene | NO.: | CDRH3 |
| | 0304-2F8 | 1145 | EIVMTQSHTLLPVTPGEPASITCRSSQS | IGHV5-51 | IGHJ6 | IGKV2-28 | IGKJ3 | 2057 | ARRGDGLYYY |
| | | | LLHSNGYNYLDWYLQKPGQSPQLLIYL | (Human) | (Human) | (Human) | (Human) | | GMDV |
| | | | GSNRASGVPDRFSGSGSGTDFTLKISR | | | | | | |
| | | | VEAEDVGVYYCMQALQTPQTFGQGT | | | | | | |
| | | | KVDIK | | | | | | |
| | 0304-3H3 | 1146 | DIVMTQSPATLSVSPEERATLSCRASQ | IGHV4-59 | IGHJ2 | IGKV3-15 | IGKJ1 | 2058 | ARDRIAPVGK |
| | | | SVSSNLAWYQQKPGQAPRLLIYGAST | (Human) | (Human) | (Human) | (Human) | | FFGWYFDL |
| | | | RATGIPARFSGSGSGTEFTLTISSLQSE | | | | | | |
| | | | DFAVYYCQQYNKWPPWTFGQGTKV | | | | | | |
| | | | DIK | | | | | | |
| | 0304- | 1147 | EIVLTQSPDSLAVSLGERATINCRSSQS | IGHV3-64 | IGHJ4 | IGKV4-1 | IGKJ3 | 2059 | ARSSSRGFDY |
| | 4A10 | | VLYSSNNKNYLAWYQQKPGQPPKVLI | (Human) | (Human) | (Human) | (Human) | | |
| | | | YWASTRESGVPDRFSGSGSGTDFTLTI | | | | | | |
| | | | SSLQAEDVAVYYCQQYYSSPYAFGPGT | | | | | | |
| | | | KVDIK | | | | | | |
| | 0304-4A2 | 1148 | EIVLTQSPDSLAVSLGERATINCKSSQS | IGHV4-39 | IGHJ4 | IGKV4-1 | IGKJ1 | 2060 | ARELFTAVAG |
| | | | VLYSSNNKNYLAWYQQKPGQPPKLLI | (Human) | (Human) | (Human) | (Human) | | KGGIDY |
| | | | YWASTRESGVPDRFSGSGSGTDFTLTI | | | | | | |
| | | | SSLQAEDVAVYYCHQYYNTPRTFGQG | | | | | | |
| | | | TKVEIK | | | | | | |
| | 0317-A1 | 1149 | DIVMTQSPATLSLSPGERATLSCRASQ | IGHV3-30 | IGHJ6 | IGKV3-11 | IGKJ3 | 2061 | AKDFKGGSSS |
| | | | SVSSYLAWYQQKPGQAPRLLIYDASN | (Human) | (Human) | (Human) | (Human) | | WYTPEIEYG |
| | | | RATGIPARFSGSGSGADFTLTISSLEPE | | | | | | MDV |
| | | | DFAVYYCRQRSNWPPTFGGGTKVDIK | | | | | | |
| | 0317-A2 | 1150 | DIVMTQSPSSLSASVGDRVTITCRASQ | IGHV7-4- | IGHJ6 | IGKV1-39 | IGKJ3 | 2062 | ARLIRHEAHT |
| | | | SISSYLNWYQQEPGKAPKLLIYAASSLQ | 1 | (Human) | (Human) | (Human) | | YCSGGSCYSP |
| | | | SGVPSRFSGSGSGTDFTLTISSLQPEDF | (Human) | | | | | DYYYGMDV |
| | | | ATYYCQQSYSTPPTFGQGTKVDIK | | | | | | |
| | 0317-A3 | 1151 | DIQMTHSLILLSASVGDRVTITCRASQS | IGHV3-48 | IGHJ3 | IGKV1-39 | IGKJ1 | 2063 | ASNPPLGEPY |
| | | | ISSYLNWYQQKPGKAPKLLIYAASSLQS | (Human) | (Human) | (Human) | (Human) | | FDI |
| | | | GVPSRFSGSGSGADFTLTISSLQPEDFA | | | | | | |
| | | | TYYCQQTYRPPWTFGQGTKVDIK | | | | | | |
| | 0317-A7 | 1152 | EIVVTQSLLSLPVTPGEPASISCRSSQS | IGHV3- | IGHJ6 | IGKV2-28 | IGKJ1 | 2064 | ARWGGGMQ |
| | | | LLHSNGYNYLDWYLQKPGQSPQVLIYL | 303 | (Human) | (Human) | (Human) | | YLDV |
| | | | GSNRASGVPDRFSGSGSGTDFTLKISR | (Human) | | | | | |
| | | | VEAEDVGVYYCMQTLQTPYTFGQGTK | | | | | | |
| | | | VEIK | | | | | | |
| | 0317-A8 | 1153 | DIQMTQSPSSLSASVGDRVTITCQASQ | IGHV7-4- | IGHJ4 | IGKV1-33 | IGKJ4 | 2065 | ARAGPNYDF |
| | | | DISNYLNWYQQKPGKAPKLLIYDASNL | 1 | (Human) | (Human) | (Human) | | WSGYYQTFD |
| | | | ETGVPSRFSGSGSGTDFTFTISSLQPED | (Human) | | | | | Y |
| | | | IATYYCQQYDNLPLTFGGGTKVEIK | | | | | | |
| | 0317-A9 | 1154 | EIVMTQSPLSSPVTLGQPASISCRSSQS | IGHV1-24 | IGHJ6 | IGKV2-24 | IGKJ3 | 2066 | ATATAMDGY |
| | | | LVHSDGNTYLSWLQQRPGQPPRLLIY | (Human) | (Human) | (Human) | (Human) | | YYYYGMDV |
| | | | KISNRFSGVPDRFSGSGAGTDFTLKISR | | | | | | |
| | | | VEAEDVGVYYCMQATQFPYTFGQGT | | | | | | |
| | | | KVDIK | | | | | | |
| | 0317-B1 | 1155 | DIVMTHSLRLLAVSLGERATINCKSSQS | IGHV5-51 | IGHJ3 | IGKV4-1 | IGKJ3 | 2067 | ASAGSSWYG |
| | | | VLYSSNNKNYLAWYQQKPGQPPKLLI | (Human) | (Human) | (Human) | (Human) | | DAFDI |
| | | | YWASTRESGVPDRFSGSGSGTDFTLTI | | | | | | |
| | | | SSLQAEDVAVYYCQQYYSTYGSFGGG | | | | | | |
| | | | TKVDIK | | | | | | |
| | 0317-C4 | 1156 | QAVVTQPPSVSAAPGQKVTISCSGSSS | IGHV1-24 | IGHJ5 | IGLV1-51 | IGU3 | 2068 | ATATIFGVAN |
| | | | NIGNNYVSWYQQLPGTAPKLLIYDNN | (Human) | (Human) | (Human) | (Human) | | NWFDP |
| | | | KRPSGIPDRFSGSKSGTSATLGITGLQT | | | | | | |
| | | | GDEADYYCGTWDSSLSVVVFGGGTKL | | | | | | |
| | | | TVL | | | | | | |
| | 0317-C9 | 1157 | QAVVTQPPSASGTPGQRVTISCSGSSS | IGHV3-30 | IGHJ6 | IGLV1-44 | IGU3 | 2069 | AKDLGYYDILT |
| | | | NIGSNTVNWYQQLPGTAPKLLIYSNN | (Human) | (Human) | (Human) | (Human) | | GQLGGYYYYY |
| | | | QRPSGVPDRFSGSKSGTSASLAISGLQ | | | | | | GMDV |
| | | | SEDEADYYCAAWDDSLNGVVFGGGT | | | | | | |
| | | | KLTVL | | | | | | |
| | 10C10 | 1158 | DIQLTQSPSSLSASVRNRVTITCRASQG | IGHV3-7 | IGHJ3 | IGKV1-17 | IGKJ4 | 2070 | ARDWDYDILT |
| | | | IKNDLCWYQQKPGKAPKRLIYAASSLQ | (Human) | (Human) | (Human) | (Human) | | GSWFGAFDI |
| | | | SGVPSRFSGSGSGTEFTLTISSLQPEDF | | | | | | |
| | | | ATYYCLQHNNYPLTFGGGTNEEIK | | | | | | |
| | 1A09 | 1159 | QAVVTQESALTTSPGETVTLTCRSSTG | IGHV2-9- | IGHJ4 | IGLV1 | IGU1 | 2071 | ARKDYYYGSS |
| | | | AVTTSNYANWVQEKPDHLFTGLIGGT | 1 | (Mouse) | (Mouse) | (Mouse) | | YAMDY |
| | | | NNRAPGVPARFSGSLIGDKAALTITGA | (Mouse) | | | | | |
| | | | QTEDEAIYFCALWYSNHWVFGGGTKL | | | | | | |
| | | | TVL | | | | | | |
| | 1A10 | 1160 | DIQMTQSPSSLSASLGDRVTISCRASQ | IGHV1-76 | IGHJ2 | IGKV10-96 | IGKJ2 | 2072 | ARDYGSSYVD |
| | | | DISNYLNWYQQKPDGTVKLLIFYTSRL | (Mouse) | (Mouse) | (Mouse) | (Mouse) | | YFDY |
| | | | HSGVPSRFSGSGSGTDYSLTISNLEQE | | | | | | |
| | | | DIATYFCQQGNTLPYTFGGGTKLEIK | | | | | | |
| | 1A12 | 1161 | DIKMTQSPSSMYASLGERVTITCKASQ | IGHV14-4 | IGHJ4 | IGKV14- | IGKJ1 | 2073 | STGGYGNYV |
| | | | DINSFLSWFQQKPGKSPKTLIYRANRL | (Mouse) | (Mouse) | 111 | (Mouse) | | DAMDY |
| | | | VDGVPSRFSGSGSGQDYSLTISSLDYE | | | (Mouse) | | | |
| | | | DMGIYYCLQYDEFRTFGGGTKLEIK | | | | | | |
| | 1B07 | 1162 | QAVVTQESALTTSPGETVTLTCRSSTG | IGHV2-9- | IGHJ4 | IGLV1 | IGU3 | 2074 | ARKDYYGSSS |
| | | | AVTTSNYANWVQEKPDHLITGLIGGT | 1 | (Mouse) | (Mouse) | (Mouse) | | NVMDY |
| | | | NNRVPGVPARFSGSLIGDKAALTITGA | (Mouse) | | | | | |
| | | | QTEDEAIYFCALWYSNQFIFGSGTKVT | | | | | | |
| | | | VL | | | | | | |
| | 1610 | 1163 | QAVVTQESALTTSPGETVTLTCRSSTG | IGHV2-9- | IGHJ4 | IGLV1 | IGU1 | 2075 | ARKDYYGSSL |
| | | | AVTTSNYANWVQEKPDHLFTGLIGGT | 1 | (Mouse) | (Mouse) | (Mouse) | | AMDY |
| | | | NNRAPGVPARFSGSLIGDKAALTITGA | (Mouse) | | | | | |
| | | | QTEDEAIYFCALWYSNHWVFGGGTKL | | | | | | |
| | | | TVL | | | | | | |
| | 1C05 | 1164 | DIVLTQSPASLAVSLGQRATISCRASES | IGHV1-42 | IGHJ4 | IGKV3-10 | IGKJ5 | 2076 | ARRNYDLYYY |
| | | | VDSYGTSFMHWYQQKPGQPPKLLIYL | (Mouse) | (Mouse) | (Mouse) | (Mouse) | | AMDY |
| | | | ASNLESGVPARFSGSGSRTDFTLTIDPV | | | | | | |
| | | | EADDAATYYCQQNNEDPLTFGAGTKL | | | | | | |
| | | | ELK | | | | | | |
| | 1C06 | 1165 | DVVMTQTPLSLPVSLGDQASISCRSSQ | IGHV1-7 | IGHJ4 | IGKV1-117 | IGKJ1 | 2077 | ARSDYYGSSY |
| | | | SIVHSNGNTYLEWYLQKPGQSPKLLIY | (Mouse) | (Mouse) | (Mouse) | (Mouse) | | VGYAMDY |
| | | | KVSNRFSGVPDRFSGSGSGTDFTLKIS | | | | | | |
| | | | RVEAEDLGVYYCFQGSHVPPTFGGGT | | | | | | |
| | | | KLEIK | | | | | | |
| | 1C07 | 1166 | DIVLTQSPASLAVSLGQRATISCRASEG | IGHV1-42 | IGHJ4 | IGKV3-10 | IGKJ5 | 2078 | ARRNYDLYYY |
| | | | VDSYGSSFMHWYQQKPGQPPKLLIYL | (Mouse) | (Mouse) | (Mouse) | (Mouse) | | AMDY |
| | | | ASNLESGVPARFSGSGSGTDFTLTIDP | | | | | | |
| | | | VEADDAATYYCQQNNEDPLTFGAGTK | | | | | | |
| | | | LELK | | | | | | |
| | 1D04 | 1167 | DIKMTQSPSSMYASLGERVTITCKASQ | IGHV14-4 | IGHJ4 | IGKV14- | IGKJ1 | 2079 | STGGYGNYV |
| | | | DINSFLSWFQQKPGKSPKTLIYRANRL | (Mouse) | (Mouse) | 111 | (Mouse) | | DAMDY |
| | | | VDGVPSRFSGSGSGQDYSLTISSLEYED | | | (Mouse) | | | |
| | | | MGIYYCLQYDEFRTFGGGTKLEIK | | | | | | |
| | 1D05 | 1168 | QAVVTQESALTTSPGETVTLTCRSSTG | IGHV2-9- | IGHJ4 | IGLV1 | IGU3 | 2080 | ARKDYYGSSS |
| | | | AVTTSNYANWVQEKPDHLITGLIGGT | 1 | (Mouse) | (Mouse) | (Mouse) | | NVMDY |
| | | | NNRVPGVPARFSGSLIGDKAALTITGA | (Mouse) | | | | | |
| | | | QTEDKAIYFCALWYSNQFIFGSGTKVT | | | | | | |
| | | | VL | | | | | | |
| | 1E02 | 1169 | DIVLTQSPASLAVSLGQRATISCRASEG | IGHV1-42 | IGHJ4 | IGKV3-10 | IGKJ5 | 2081 | SRRNYDLYYY |
| | | | VDSYGSSFMHWYQQKPGQAPKLLIYL | (Mouse) | (Mouse) | (Mouse) | (Mouse) | | AMDY |
| | | | ASNLESGVPARFSGSGSRTDFTLTIDPV | | | | | | |
| | | | EADDAAIYYCQQNNEDPLTFGAGTKL | | | | | | |
| | | | EIK | | | | | | |
| | 1E07 | 1170 | DIVLTQSPASLAVSLGQRATISCRASES | IGHV1-42 | IGHJ3 | IGKV3-1 | IGKJ1 | 2082 | ARGAGAY |
| | | | VEYYGTSLMQWFQQKPGQPPKLLIFA | (Mouse) | (Mouse) | (Mouse) | (Mouse) | | |
| | | | ASNVESGVPARFGGSGSGTDFSLNIHP | | | | | | |
| | | | VEEDDIAMYFCQQSRKVPWTFGGGT | | | | | | |
| | | | KLEIK | | | | | | |
| | 1E10 | 1171 | QIVLTQSPAIMSASPGEKVTMTCSASS | IGHV1-82 | IGHJ3 | IGKV4-59 | IGKJ2 | 2083 | ARDHGPA |
| | | | SVSYMHWYQQKSGTSPKRWIYDTSKL | (Mouse) | (Mouse) | (Mouse) | (Mouse) | | |
| | | | ASGVPARFSGSGSGTSYSLTISSMEAE | | | | | | |
| | | | DAATYYCQQWSSNPPTFGSGTKLEIK | | | | | | |
| | 1H06 | 1172 | DIVLTQSPASLAVSLGQRATISCRASES | IGHV1-42 | IGHJ3 | IGKV3-1 | IGKJ1 | 2084 | ARGAGAY |
| | | | VEYYGTSLMQWFQQKPGQPPKLLIFA | (Mouse) | (Mouse) | (Mouse) | (Mouse) | | |
| | | | ASNVESGVPARFGGSGSGTDFSLNIHP | | | | | | |
| | | | VEEDDIATYFCQQSRKVPWTFGGGTK | | | | | | |
| | | | LEIK | | | | | | |
| | 1H10 | 1173 | QAVVTQESALTTSPGETVTLTCRSSTG | IGHV2-9- | IGHJ2 | IGLV1 | IGU1 | 2085 | ARISYYDYEG |
| | | | AVTTSNYANWVQEKPDHLFTGLIGGT | 1 | (Mouse) | (Mouse) | (Mouse) | | VDY |
| | | | NNRAPGVPARFSGSLIGDKAALTITGA | (Mouse) | | | | | |
| | | | QTEDEAIYFCALWYSNHWVFGGGTKL | | | | | | |
| | | | TVL | | | | | | |
| | 1M-1D2 | 1174 | QAVVTQPPSASGTPGQRVTISCSGSSS | IGHV3-64 | IGHJ4 | IGLV1-47 | IGU3 | 2086 | ARGAEYYDF |
| | | | NIGSNYVYWYQQLPGTAPKLLIYNNN | (Human) | (Human) | (Human) | (Human) | | WSGYYSAYFD |
| | | | QRPSGVPDRFSGSKSGTSASLAISGLRS | | | | | | Y |
| | | | EDEADYYCAAWDDSLIYVLFGGGTKLT | | | | | | |
| | | | VL | | | | | | |
| | 2B04 | 1175 | QAVVTQESALTTSPGETVTLTCRSSTG | IGHV2-9- | IGHJ4 | IGLV1 | IGU1 | 2087 | ARKDYYGRYY |
| | | | AVTTSNYANWVQEKPDHLFTGLIGGT | 1 | (Mouse) | (Mouse) | (Mouse) | | GMDY |
| | | | NNRAPGVPARFSGSLIGDKAALTITGA | (Mouse) | | | | | |
| | | | QTEDEAIYFCALWYNNHWVFGGGTK | | | | | | |
| | | | LTVL | | | | | | |
| | 2C02 | 1176 | QIVLTQSPAIMSASPGEKVTMTCSASS | IGHV1-74 | IGHJ1 | IGKV4-55 | IGKJ5 | 2088 | AILDSYWYFD |
| | | | SVSYMYWYQQKPGSSPRLLIYDTSNLA | (Mouse) | (Mouse) | (Mouse) | (Mouse) | | V |
| | | | SGVPIRFSGSGSGTSYSLTISRMEAEDA | | | | | | |
| | | | ATYYCQQWSSYPLTFGAGTKLELK | | | | | | |
| | 2C03 | 1177 | DVVMTQTPLSLPVSLGDQASISCRSSQ | IGHV1-82 | IGHJ1 | IGKV1-117 | IGKJ2 | 2089 | ARKSYGYWH |
| | | | SIVYSNGNTYLEWYLQKPGQSPKLLIYK | (Mouse) | (Mouse) | (Mouse) | (Mouse) | | FDV |
| | | | VSNRFSGVPDRFSGSGSGTDFTLKISG | | | | | | |
| | | | VEAEDLGVYYCFQGSHVPYTFGGGTK | | | | | | |
| | | | LEIK | | | | | | |
| | 2C04 | 1178 | DIVLTQSQASLAVSLGQRATISCRASES | IGHV1-26 | IGHJ2 | IGKV3-1 | IGKJ1 | 2090 | AAGKGDY |
| | | | VEYYGTSLMQWYQQKPGQPPKLLIYA | (Mouse) | (Mouse) | (Mouse) | (Mouse) | | |
| | | | ASNVESGVPARFSGSGSGTDFSLNIHP | | | | | | |
| | | | VEEDDIAMYFCQQSRKVPWTFGGGT | | | | | | |
| | | | KLEIK | | | | | | |
| | 2D01 | 1179 | QIVLTQSPAIMSASPGEKVTMTCSASS | IGHV1-74 | IGHJ1 | IGKV4-55 | IGKJ5 | 2091 | AILDSYWYFD |
| | | | SVSYMYWYQQKPGSSPRLLIYDTSNLA | (Mouse) | (Mouse) | (Mouse) | (Mouse) | | V |
| | | | SGVPIRFSGSGSGTSYSLTISRMEAEDA | | | | | | |
| | | | ATYYCQQWSSYPLTFGAGTKLELK | | | | | | |
| | 2D08 | 1180 | DIVLTQSQASLAVSLGQRATISCRASES | IGHV1-26 | IGHJ2 | IGKV3-1 | IGKJ1 | 2092 | AAGKGDY |
| | | | VEYYGTSLMQWYQQKPGQPPKLLIYA | (Mouse) | (Mouse) | (Mouse) | (Mouse) | | |
| | | | ASNVESGVPARFSGSGSGTDFSLNIHP | | | | | | |
| | | | VEEDDIAMYFCQQSRKVPWTFGGGT | | | | | | |
| | | | KLEIK | | | | | | |
| | 2D11 | 1181 | DIVLTQSPASLAVSLGQRATISCRASES | IGHV1-26 | IGHJ3 | IGKV3-2 | IGKJ1 | 2093 | ARKGDGYYG |
| | | | VDNYGISFMNWFQQKPGQPPKLLIYA | (Mouse) | (Mouse) | (Mouse) | (Mouse) | | GFAY |
| | | | ASNQGSGVPARFSGSGSGTDFSLNIHP | | | | | | |
| | | | MEEDDTAMYFFQQSKEVPWTFGGGT | | | | | | |
| | | | KLEIK | | | | | | |
| | 2E06 | 1182 | DIVLTQSPASLTVSLGQRATISCKASQS | IGHV14-3 | IGHJ4 | IGKV3-4 | IGKJ2 | 2094 | ATYGSYYLYYY |
| | | | VDYGGDRYMNWYQQKPGQPPKLLIF | (Mouse) | (Mouse) | (Mouse) | (Mouse) | | AMNY |
| | | | AASNLESGIPARFSGSGSGTDFTLNIHP | | | | | | |
| | | | VEEEDAATYYCQQSNEDPYTFGGGTK | | | | | | |
| | | | LEIK | | | | | | |
| | 2E10 | 1183 | DIVLTQSPASLAVSLGQRATMSCRASE | IGHV1-76 | IGHJ4 | IGKV3-4 | IGKJ5 | 2095 | ARYDGNLYYY |
| | | | SVDSYGNGFMHWYQQKPGQPPKLLI | (Mouse) | (Mouse) | (Mouse) | (Mouse) | | AMDY |
| | | | YLASNLESGVPARFSGSGSRTDFSLTID | | | | | | |
| | | | PVEADDAAIYYCQQNNEDPLTFGAGT | | | | | | |
| | | | KLELK | | | | | | |
| | 2F04 | 1184 | DIVLTQSPAILSVSPGERVSFSCRASQN | IGHV1-55 | IGHJ2 | IGKV5-48 | IGKJ5 | 2096 | ARWDFYGSR |
| | | | IGTIIHWYQQRTNGSPRLLIKYASESVS | (Mouse) | (Mouse) | (Mouse) | (Mouse) | | TFDY |
| | | | GIPSRFSGSGSGTDFTLSINSVESEDIA | | | | | | |
| | | | DYYCQQSSSWPLTFGAGTKLELK | | | | | | |
| | 2H04 | 1185 | DIVLTQSPAILSVSPGERVSFSCRASQN | IGKV5-48 | IGKJ5 | IGHV1-55 | IGHJ2 | 2097 | ARWDFYGSR |
| | | | IGTIIHWYQQRTNGSPRLLIKYASESVS | (Mouse) | (Mouse) | (Mouse) | (Mouse) | | TFDY |
| | | | GIPSRFSGSGSGTDFTLSINSVESEDIA | | | | | | |
| | | | DYYCQQSSSWPLTFGAGTKLELK | | | | | | |
| | 2M- | 1186 | QSALTQPHSVSESPGKTVTISCTGSSGS | IGHV3-66 | IGHJ6 | IGLV6-57 | IGU3 | 2098 | ARATWLRGV |
| | 10B11 | | IASNYVQWYQQRPGSAPTTVIYEDNQ | (Human) | (Human) | (Human) | (Human) | | MDVW |
| | | | RPSGVPDRFSGSIDSSSNSASLTISGLK | | | | | | |
| | | | TEDEADYYCQSYDSSNHWVFGGGTKLT | | | | | | |
| | | | VL | | | | | | |
| | 2M-12D7 | 1187 | AIRMTQSPSSLSASVGDRVTITCRASQ | IGHV3-9 | IGHJ3 | IGKV1-39 | IGKJ3 | 2099 | AKDVRYCSST |
| | | | SITGYLNWYQQKPGKAPKLLISAASSL | (Human) | (Human) | (Human) | (Human) | | SCYFSAFDI |
| | | | QSGVPSRFSGSGSGTDFTLTISSLQPED | | | | | | |
| | | | FATYYCQQSYSTPRTFGPGTKVEIK | | | | | | |
| | 2M-13A3 | 1188 | AIRMTQSPDSLAVSLGERATINCKSSQ | IGHV3-30 | IGHJ3 | IGKV4-1 | IGKJ5 | 2100 | ARGGGSYYY |
| | | | SVLYSPNNKNYLAWYQQKPGQPPKLL | (Human) | (Human) | (Human) | (Human) | | WFDP |
| | | | IYWASTRESGVPDRFSGSGSGTDFTLTI | | | | | | |
| | | | SSLQAEDVAVYYCQQYYSTPFTFGQGT | | | | | | |
| | | | RLEIK | | | | | | |
| | 2M- | 1189 | EIVMTQSPGTLSLSPGERATLSCRASQ | IGHV4-34 | IGHJ3 | IGKV3-20 | IGKJ1 | 2101 | ARAGYSSSWY |
| | 13D11 | | SVSSSYLAWYQQKPGQAPRLLIYGASS | (Human) | (Human) | (Human) | (Human) | | GVRGVDP |
| | | | RATGIPDRFSGSGSGTDFTLTISRLEPE | | | | | | |
| | | | DFAVYYCQQYGSSRSWTFGQGTKVEI | | | | | | |
| | | | K | | | | | | |
| | 2M-14B2 | 1190 | AIRMTQSPSSLSASVGDRVTITCRASQ | IGHV3-30 | IGHJ5 | IGKV1-39 | IGKJ3 | 2102 | AKGSDIVWP |
| | | | SISSYLNWYQQKPGKAPKLLIYAASSLQ | (Human) | (Human) | (Human) | (Human) | | VGNWFDP |
| | | | SGVPSRFSGSGSGTDFTLTISSLQPEDF | | | | | | |
| | | | ATYYCQQSYSTFTLYTFGQGTKVDIK | | | | | | |
| | 2M-14E4 | 1191 | DIVMTQSPSSLSASVGDRVSITCRASQ | IGHV4-61 | IGHJ4 | IGKV1-39 | IGKJ3 | 2103 | ARVQRYYPDS |
| | | | NISNYLNWYQQKPGEAPKLLISAASSL | (Human) | (Human) | (Human) | (Human) | | SGFYGRRFDI |
| | | | QSGVPSRFGGSGSGTGFTLTINSLQPE | | | | | | |
| | | | DFATYYCQQSHSFPFTFGPGTKVDIK | | | | | | |
| | 2M-14E5 | 1192 | AIRMTQSPSFLSVSVGDRVTITCRASQ | IGHV3-30 | IGHJ4 | IGKV1-9 | IGKJ4 | 2104 | ARSGGGSYR |
| | | | GISSYLAWYQQKPGKAPKLLIYAASTL | (Human) | (Human) | (Human) | (Human) | | GPFDY |
| | | | QSGVPSRFSGSGSGTEFTLTISSLQPED | | | | | | |
| | | | FATYYCQQLNSYVTFGGGTKVEIK | | | | | | |
| | 2M-2D1 | 1193 | QSVLTQLPSASGTPGQTVTISCSGNVF | IGHV4-39 | IGHJ3 | IGLV1-47 | IGU3 | 2105 | ARGDRIQLW |
| | | | KEKSNYVYWYQQLPGTAPKLLIYRNN | (Human) | (Human) | (Human) | (Human) | | LLDAFDI |
| | | | HRPSGVPDRFSGSKSGTSPSLAISGLRS | | | | | | |
| | | | EDEADYYCAARDDSLSGWVFSGRTKL | | | | | | |
| | | | TVL | | | | | | |
| | 2M-2D4 | 1194 | EIVMTQSPATLSLSPGERATLSCRASQS | IGHV3-23 | IGHJ4 | IGKV3-11 | IGKJ3 | 2106 | AKIGLGLGGL |
| | | | VSTSLAWYQQKPGQAPRLLIYDASNR | (Human) | (Human) | (Human) | (Human) | | LRRYFDY |
| | | | ATGVPARFSGSGSGTDFTLTISSLEPDD | | | | | | |
| | | | FAVYYCQQRTNWPLFGPGTKVEIK | | | | | | |
| | 2M-2G12 | 1195 | DIQMTQSPSSLSVSAGERATLSCRASQ | IGHV3-11 | IGHJ6 | IGKV1-39 | IGKJ1 | 2107 | GTRIMITWYS |
| | | | SVSSYLIWCHQKPGKAPKLLIYDASTLQ | (Human) | (Human) | (Human) | (Human) | | RRGMDG |
| | | | TGVPSRFIGGGSGTNFTLTIISLQPEDF | | | | | | |
| | | | AAYYCQQNYSTWTIGQGSRVEMK | | | | | | |
| | 2M-4G4 | 1196 | QSVLTQPASVSGSPGQSITISCTGTSSD | IGHV1-46 | IGHJ6 | IGLV2-23 | IGU3 | 2108 | ARERGDSSGY |
| | | | VGMYNLVSWYQQHPGQAPKLMIYE | (Human) | (Human) | (Human) | (Human) | | YEIITTANRRF |
| | | | GSKRPSGVSNRFSGSKSGNTASLTISGL | | | | | | GMDV |
| | | | QAEDEAYYYCCSYAVSSTWVFGGGTK | | | | | | |
| | | | LTVL | | | | | | |
| | 2M-7E9 | 1197 | DIVMTQSPATLSLSPGERATLSCRASQ | IGHV1-69 | IGHJ6 | IGKV3-11 | IGKJ3 | 2109 | ARIPGWDRG |
| | | | SVSNFLAWYQQKPGQAPRLLIYDASN | (Human) | (Human) | (Human) | (Human) | | TDRNWNDD |
| | | | RATGIPARFSGSGSGTDFTLTISSLEPE | | | | | | |
| | | | DFAVYYCQQRSNWPPAFTFGPGTKVE | | | | | | |
| | | | IK | | | | | | |
| | 2M-8E7 | 1198 | EIVLTQSPATLSLSPGERATLSCRASQS | IGHV1-69 | IGHJ4 | IGKV3-11 | IGKJ5 | 2110 | ARTYSFDSSG |
| | | | VSSYLAWYQQKPGQAPRLLIYDASNR | (Human) | (Human) | (Human) | (Human) | | YYYDY |
| | | | ATGIPARFSGSGSGTDFTLTISSLEPED | | | | | | |
| | | | FAVYYCQQRSNWPPKITFGQGTRLEIK | | | | | | |
| | 2M-8H10 | 1199 | DIQMTQSPDSLAVSLGERATIKCKSSQ | IGHV3-30 | IGHJ5 | IGKV4-1 | IGKJ1 | 2111 | ARAFYDSNW |
| | | | SVLHSSNNKNYLAWYQQKAGQPPSLL | (Human) | (Human) | (Human) | (Human) | | SVGSYFDS |
| | | | LYWASTRESGVPDRFSGSGSGTDFTLT | | | | | | |
| | | | ISSLQAEDVAVYYCQQYYNNQWTFGQ | | | | | | |
| | | | GTKVDIK | | | | | | |
| | 2M-9F10 | 1200 | AIRMTQSPSSLSASVGDRVTISCRATQ | IGHV3-9 | IGHJ4 | IGKV1-39 | IGKJ3 | 2112 | AKDSVRREYT |
| | | | NINYFLNWYQQKPGRAPKLLIYAASGL | (Human) | (Human) | (Human) | (Human) | | HARVPFDN |
| | | | QSGVPSRFSGSGTGRVFTLTINSLQPD | | | | | | |
| | | | DFATYYCQQSFVSPRTFGQGTKVDIK | | | | | | |
| | 2M-9H1 | 1201 | DIVMTQSPSSLSAFVGDRVTITCRASR | IGHV3-30 | IGHJ4 | IGKV1-17 | IGKJ4 | 2113 | AKSSKIFYLGE |
| | | | DIGGDLSWFQQKPGKAPERLIYAASSL | (Human) | (Human) | (Human) | (Human) | | SREVDY |
| | | | ESGVPSRFSGSGSATEFALTITSLQPED | | | | | | |
| | | | FATYYCLQHKSYPLTFGGGTKVDIK | | | | | | |
| | 31B5 | | ND | ND | ND | ND | ND | 2114 | ARVEYYYGSG |
| | | | | | | | | | SYMPWYFDL |
| | 31B9 | | ND | ND | ND | ND | ND | 2115 | ATYYYDSSGYS |
| | | | | | | | | | YGMDV |
| | 32D4 | | ND | ND | ND | ND | ND | 2116 | TEEGSGSEGP |
| | | | | | | | | | IEFDY |
| | 413-2 | | ND | ND | ND | ND | ND | 2117 | ARDNNYRNY |
| | | | | | | | | | YYYMDVW |
| | 414-1 | | ND | ND | ND | ND | ND | 2118 | ARGALGCSST |
| | | | | | | | | | SCYPNNFDY |
| | | | | | | | | | W |
| | 47D11 | | ND | ND | ND | ND | ND | 2119 | ND |
| | 4A8 | 1202 | EIVMTQSPLSSPVTLGQPASISCRSSQS | IGHV1-24 | IGHJ6 | IGKV2-24 | IGKJ3 | 2120 | ATSTAVAGTP |
| | | | LVHSDGNTYLSWLQQRPGQPPRLLIY | (Human) | (Human) | (Human) | (Human) | | DLFDYYYGM |
| | | | KISNRFSGVPDRFSGSGAGTDFTLKISR | | | | | | DV |
| | | | VEAEDVGVYYCTQATQFPYTFGQGTK | | | | | | |
| | | | VDIK | | | | | | |
| | 505-3 | | ND | ND | ND | ND | ND | 2121 | ARDFISRPRG |
| | | | | | | | | | YRW |
| | 505-5 | | ND | ND | ND | ND | ND | 2122 | ARDFISRPRG |
| | | | | | | | | | YRW |
| | 505-8 | | ND | ND | ND | ND | ND | 2123 | ARGQDDFWS |
| | | | | | | | | | MNWFDPW |
| | 515-1 | | ND | ND | ND | ND | ND | 2124 | ARDFISRPRG |
| | | | | | | | | | YRW |
| | 515-5 | | ND | ND | ND | ND | ND | 2125 | TRGSQWATI |
| | | | | | | | | | NDARFDYW |
| | 553-15 | | ND | ND | ND | ND | ND | 2126 | ARVWYYYGP |
| | | | | | | | | | RDYW |
| | 553-49 | | ND | ND | ND | ND | ND | 2127 | AKDSSSWYNY |
| | | | | | | | | | YGMDVW |
| | 553-60 | | ND | ND | ND | ND | ND | 2128 | ARVFRGSQY |
| | | | | | | | | | WFDPW |
| | 553-63 | | ND | ND | ND | ND | ND | 2129 | ARMEAPKLTL |
| | | | | | | | | | DPW |
| | 8D2 | 1203 | DIQMTQSPSSLSASVGDRVTITCRASQ | IGHV3-7 | IGHJ3 | IGKV1-17 | IGKJ4 | 2130 | ARDWDYDILT |
| | | | GIRNDLGWYQQKPGKAPKRLIYAASSL | (Human) | (Human) | (Human) | (Human) | | GSWFGAFDI |
| | | | QSGVPSRFSGSGSGTEFTLTISSLQPED | | | | | | |
| | | | FATYYCLQHNSYPLTFGGGTKVEIK | | | | | | |
| | 8D9 | 1204 | SYELTQPPSVSVSPGQTASITCSGDKLG | IGHV3-7 | IGHJ4 | IGLV3-1 | IGU3 | 2131 | ARPTIGYSYGS |
| | | | DKYACWYRQKPGQSPVLVIYQDSKRP | (Human) | (Human) | (Human) | (Human) | | DY |
| | | | SGIPERFSGSNSGNTATLTISGTQAMD | | | | | | |
| | | | EADYYCQAWDSSTGVFGGGTKLTVL | | | | | | |
| | 9A1 | 1205 | DIVMTQSPATLSASPGERVTLSCRASQ | IGHV3-30 | IGHJ6 | IGKV3-15 | IGKJ3 | 2132 | AKVSAIFWLG |
| | | | NIRNNLAWYQQKPGQAPRLLIHGAST | (Human) | (Human) | (Human) | (Human) | | QGLSPIDV |
| | | | RAAGAPARFSGSGSDTQFTLTVSSLQS | | | | | | |
| | | | EDFAVYYCHQYSKWPVTFGGGTKVDI | | | | | | |
| | | | K | | | | | | |
| | Ab_510A | | ND | IGHV2-70 | IGHJ4 | IGKV2-28 | IGKJ1 | 2133 | ARVQVAAAG |
| | | | | (Human) | (Human) | (Human) | (Human) | | SPYDY |
| | Ab_510A5 | | ND | IGHV3-9 | IGHJ4 | IGKV1-39 | IGKJ2 | 2134 | AKDRGYEILTP |
| | | | | (Human) | (Human) | (Human) | (Human) | | ASFDY |
| | Ab_510D7 | | ND | IGHV1-69 | IGHJ4 | IGKV3-20 | IGKJ2 | 2135 | ATGRYTYGYG |
| | | | | (Human) | (Human) | (Human) | (Human) | | YYFDY |
| | Ab_510G | | ND | IGHV4-31 | IGHJ4 | IGKV1-33 | IGKJ4 | 2136 | ARDYGGNSN |
| | | | | (Human) | (Human) | (Human) | (Human) | | YFHY |
| | Ab_510H10 | | ND | IGHV2-70 | IGHJ4 | IGKV1-39 | IGKJ2 | 2137 | ARIQRGIAAD |
| | | | | (Human) | (Human) | (Human) | (Human) | | Y |
| | Ab_510H2 | | ND | IGHV3-66 | IGHJ4 | IGKV1-9 | IGKJ4 | 2138 | ARDKWEGTF |
| | | | | (Human) | (Human) | (Human) | (Human) | | DY |
| | Ab_510H | | ND | IGHV3-66 | IGHJ6 | IGKV3-11 | IGKJ1 | 2139 | AETGWDGM |
| | | | | (Human) | (Human) | (Human) | (Human) | | DV |
| | Ab_510H7 | | ND | IGHV4-59 | IGHJ5 | IGKV1D-13 | IGKJ4 | 2140 | ARHCPWQQL |
| | | | | (Human) | (Human) | (Human) | (Human) | | VSNWFDP |
| | Ab_511A1 | | ND | IGHV4-31 | IGHJ6 | IGKV3-15 | IGKJ1 | 2141 | AREKIRSIAAA |
| | | | | (Human) | (Human) | (Human) | (Human) | | GTVYYYGMD |
| | | | | | | | | | V |
| | Ab_511A5 | | ND | IGHV4-31 | IGHJ2 | IGLV3-21 | IGU1 | 2142 | ARIYRGTMVV |
| | | | | (Human) | (Human) | (Human) | (Human) | | VFSDLHWYFD |
| | | | | | | | | | L |
| | Ab_511B11 | | ND | IGHV3-7 | IGHJ4 | IGLV1-40 | IGU1 | 2143 | AGLFWYGGY |
| | | | | (Human) | (Human) | (Human) | (Human) | | FDY |
| | Ab_511B | | ND | IGHV4-59 | IGHJ5 | IGKV1D-12 | IGKJ4 | 2144 | ASTYWDSSGY |
| | | | | (Human) | (Human) | (Human) | (Human) | | YYGVDY |
| | Ab_511D11 | | ND | IGHV1-18 | IGHJ6 | IGKV3-20 | IGKJ2 | 2145 | AVLDYCSGGS |
| | | | | (Human) | (Human) | (Human) | (Human) | | SSSGYYNYGM |
| | | | | | | | | | DV |
| | Ab_511E5 | | ND | IGHV1-2 | IGHJ2 | IGLV1-40 | IGU2 | 2146 | ARDSLFSRVD |
| | | | | (Human) | (Human) | (Human) | (Human) | | WYFDL |
| | Ab_511E7 | | ND | IGHV5-51 | IGHJ4 | IGKV1-33 | IGKJ3 | 2147 | ALAVGRGIPT |
| | | | | (Human) | (Human) | (Human) | (Human) | | SYFDY |
| | Ab_511E9 | | ND | IGHV1-18 | IGHJ4 | IGKV6-21 | IGKJ2 | 2148 | AREGAGLIIAY |
| | | | | (Human) | (Human) | (Human) | (Human) | | DY |
| | Ab_511G5 | | ND | IGHV1-46 | IGHJ4 | IGLV1-47 | IGU3 | 2149 | ARDGALYSNS |
| | | | | (Human) | (Human) | (Human) | (Human) | | PTEFDY |
| | Ab_511G7 | | ND | IGHV3-33 | IGHJ4 | IGKV1-33 | IGKJ4 | 2150 | AKGGNYGDY |
| | | | | (Human) | (Human) | (Human) | (Human) | | LRGFDY |
| | Ab_511H11 | | ND | IGHV3-33 | IGHJ6 | IGKV1-39 | IGKJ1 | 2151 | VRGDHSSGW |
| | | | | (Human) | (Human) | (Human) | (Human) | | YGTYYYYMDV |
| | Ab_511H7 | | ND | IGHV3-23 | IGHJ6 | IGLV1-47 | IGU3 | 2152 | ARGLQYYYDT |
| | | | | (Human) | (Human) | (Human) | (Human) | | SGYYKDSYYY |
| | | | | | | | | | GVDV |
| | Ab_51A1 | | ND | IGHV3-66 | IGHJ3 | IGKV1-9 | IGKJ5 | 2153 | ARDLNIAGGF |
| | | | | (Human) | (Human) | (Human) | (Human) | | DI |
| | Ab_51A3 | | ND | IGHV1-18 | IGHJ4 | IGKV3-11 | IGKJ5 | 2154 | ARDLAWFGE |
| | | | | (Human) | (Human) | (Human) | (Human) | | LSESPIEY |
| | Ab_51D2 | | ND | IGHV2-5 | IGHJ3 | IGKV1-39 | IGKJ4 | 2155 | AHRLAPDYDF |
| | | | | (Human) | (Human) | (Human) | (Human) | | LTGYYNGDD |
| | | | | | | | | | AFDV |
| | Ab_51D3 | | ND | IGHV3-66 | IGHJ6 | IGKV1-33 | IGKJ2 | 2156 | AREGLLVGPT |
| | | | | (Human) | (Human) | (Human) | (Human) | | GRGLGMDV |
| | Ab_51D4 | | ND | IGHV2-70 | IGHJ4 | IGKV1-39 | IGKJ2 | 2157 | ARMVVRGV |
| | | | | (Human) | (Human) | (Human) | (Human) | | MLDY |
| | Ab_51D7 | | ND | IGHV5-51 | IGHJ3 | IGKV1-39 | IGKJ2 | 2158 | ATRTGWTND |
| | | | | (Human) | (Human) | (Human) | (Human) | | AFDI |
| | Ab_51E10 | | ND | IGHV1-18 | IGHJ2 | IGKV4-1 | IGKJ4 | 2159 | ARARQLVLN |
| | | | | (Human) | (Human) | (Human) | (Human) | | WYFDL |
| | Ab_51E12 | | ND | IGHV3-7 | IGHJ4 | IGKV1-8 | IGKJ2 | 2160 | ARLMYYYGNF |
| | | | | (Human) | (Human) | (Human) | (Human) | | DY |
| | Ab_51E7 | | ND | IGHV3-13 | IGHJ2 | IGKV1-39 | IGKJ1 | 2161 | ARVGYYGSGS |
| | | | | (Human) | (Human) | (Human) | (Human) | | YPLYWYFDL |
| | Ab_51F11 | | ND | IGHV1-69 | IGHJ4 | IGKV3-20 | IGKJ2 | 2162 | ATGRYTYGYG |
| | | | | (Human) | (Human) | (Human) | (Human) | | YYFDY |
| | Ab_52C1 | | ND | IGHV3-66 | IGHJ4 | IGLV1-40 | IGU3 | 2163 | ARLASDGSGS |
| | | | | (Human) | (Human) | (Human) | (Human) | | YLDYFDY |
| | Ab_52C6 | | ND | IGHV1-69 | IGHJ6 | IGLV8-61 | IGU2 | 2164 | ATDGGGGSY |
| | | | | (Human) | (Human) | (Human) | (Human) | | YYAHYYYGM |
| | Ab_52F7 | | ND | IGHV3-9 | IGHJ6 | IGKV2D-30 | IGKJ2 | 2165 | AKDIGVMVP |
| | | | | (Human) | (Human) | (Human) | (Human) | | GVTPYGMDV |
| | Ab_52G9 | | ND | IGHV3-66 | IGHJ6 | IGKV1-33 | IGKJ1 | 2166 | ARDPMRPG |
| | | | | (Human) | (Human) | (Human) | (Human) | | MDV |
| | Ab_53C10 | | ND | IGHV3-43 | IGHJ4 | IGLV3-21 | IGU1 | 2167 | ARESPKLTGY |
| | | | | (Human) | (Human) | (Human) | (Human) | | FDY |
| | Ab_53C5 | | ND | IGHV1-69 | IGHJ4 | IGKV1-5 | IGKJ2 | 2168 | ARGRYTYGTE |
| | | | | (Human) | (Human) | (Human) | (Human) | | GYFDN |
| | Ab_53F12 | | ND | IGHV3-66 | IGHJ6 | IGKV3-15 | IGKJ2 | 2169 | ARDAVGSYYY |
| | | | | (Human) | (Human) | (Human) | (Human) | | GMEV |
| | Ab_53F9 | | ND | IGHV3-53 | IGHJ4 | IGKV2D-30 | IGKJ2 | 2170 | AREGLVGTTL |
| | | | | (Human) | (Human) | (Human) | (Human) | | TFDY |
| | Ab_53H3 | | ND | IGHV3-66 | IGHJ3 | IGKV1-33 | IGKJ3 | 2171 | ARYYGPQGR |
| | | | | (Human) | (Human) | (Human) | (Human) | | AFDI |
| | Ab_55A8 | | ND | IGHV1-69 | IGHJ4 | IGKV1-5 | IGKJ2 | 2172 | ARGTEYGDYD |
| | | | | (Human) | (Human) | (Human) | (Human) | | VSHD |
| | Ab_55C9 | | ND | IGHV3-53 | IGHJ4 | IGKV1-39 | IGKJ2 | 2173 | AREGLVGTAL |
| | | | | (Human) | (Human) | (Human) | (Human) | | AFDY |
| | Ab_56C12 | | ND | IGHV3-30 | IGHJ5 | IGKV1-39 | IGKJ1 | 2174 | AKDPTSLYCS |
| | | | | (Human) | (Human) | (Human) | (Human) | | GGSCYNNWF |
| | Ab_56D7 | | ND | IGHV3-66 | IGHJ6 | IGKV1-9 | IGKJ5 | 2175 | ARDLDYYGM |
| | | | | (Human) | (Human) | (Human) | (Human) | | DV |
| | Ab_56E1 | | ND | IGHV3-30 | IGHJ3 | IGLV1-44 | IGU3 | 2176 | AGGGVLVTS |
| | | | | (Human) | (Human) | (Human) | (Human) | | DPDAFDI |
| | Ab_56H11 | | ND | IGHV4-4 | IGHJ4 | IGLV1-44 | IGU3 | 2177 | AGEQHIVTTII |
| | | | | (Human) | (Human) | (Human) | (Human) | | DY |
| | Ab_56H3 | | ND | IGHV3-66 | IGHJ4 | IGKV3-20 | IGKJ1 | 2178 | ARDYGDYYFD |
| | | | | (Human) | (Human) | (Human) | (Human) | | Y |
| | Ab_57A6 | | ND | IGHV5-51 | IGHJ4 | IGLV3-19 | IGU3 | 2179 | ARQESGWSF |
| | | | | (Human) | (Human) | (Human) | (Human) | | DY |
| | Ab_57A8 | | ND | IGHV3-23 | IGHJ4 | IGKV1-5 | IGKJ4 | 2180 | AKGQRGSPD |
| | | | | (Human) | (Human) | (Human) | (Human) | | FFDY |
| | Ab_57A9 | | ND | IGHV1-3 | IGHJ4 | IGKV4-1 | IGKJ1 | 2181 | ARAGWELNY |
| | | | | (Human) | (Human) | (Human) | (Human) | | |
| | Ab_57B8 | | ND | IGHV3-53 | IGHJ4 | IGKV1-9 | IGKJ5 | 2182 | ARDLVTWGL |
| | | | | (Human) | (Human) | (Human) | (Human) | | DY |
| | Ab_57C4 | | ND | IGHV3-15 | IGHJ5 | IGKV1-39 | IGKJ4 | 2183 | STTNDYGDYS |
| | | | | (Human) | (Human) | (Human) | (Human) | | ANY |
| | Ab_57E11 | | ND | IGHV1-8 | IGHJ5 | IGLV1-44 | IGU3 | 2184 | ARGLWFGDL |
| | | | | (Human) | (Human) | (Human) | (Human) | | TRTKYNWFD |
| | | | | | | | | | P |
| | Ab_57F7 | | ND | IGHV4-34 | IGHJ6 | IGLV1-44 | IGU3 | 2185 | ARDDSSSSGV |
| | | | | (Human) | (Human) | (Human) | (Human) | | GTGMDV |
| | Ab_57G9 | | ND | IGHV2-70 | IGHJ4 | IGKV1-39 | IGKJ2 | 2186 | ARITPHLVYDY |
| | | | | (Human) | (Human) | (Human) | (Human) | | |
| | Ab_58A4 | | ND | IGHV4-59 | IGHJ5 | IGKV3-20 | IGKJ1 | 2187 | ARTLGAYYDIL |
| | | | | (Human) | (Human) | (Human) | (Human) | | TGFRTPGGW |
| | | | | | | | | | FAP |
| | Ab_58D2 | | ND | IGHV3-11 | IGHJ2 | IGKV3-11 | IGKJ2 | 2188 | ASPLLSHNYG |
| | | | | (Human) | (Human) | (Human) | (Human) | | SGSYYNVYWY |
| | | | | | | | | | FEL |
| | Ab_58G1 | | ND | IGHV3-53 | IGHJ6 | IGKV1D-12 | IGKJ4 | 2189 | ARDLENGGL |
| | | | | (Human) | (Human) | (Human) | (Human) | | DV |
| | Ab_58G6 | | ND | IGHV1-58 | IGHJ3 | IGKV3-20 | IGKJ1 | 2190 | AAPNCNSTTC |
| | | | | (Human) | (Human) | (Human) | (Human) | | HDGFDI |
| | Ab_59A2 | | ND | IGHV3-66 | IGHJ4 | IGKV1-33 | IGKJ3 | 2191 | ARDLPLHGDY |
| | | | | (Human) | (Human) | (Human) | (Human) | | FDY |
| | Ab_59D6 | | ND | IGHV4-34 | IGHJ4 | IGLV1-40 | IGU2 | 2192 | ARHRRDYITM |
| | | | | (Human) | (Human) | (Human) | (Human) | | IVRPTRLWAF |
| | | | | | | | | | DY |
| | Ab_81A11 | | ND | IGHV1-69 | IGHJ2 | IGLV1-40 | IGU2 | 2193 | AREAGTTDW |
| | | | | (Human) | (Human) | (Human) | (Human) | | YFDL |
| | Ab_81C3 | | ND | IGHV4-39 | IGHJ4 | IGLV2-14 | IGU1 | 2194 | ARHPRFSWR |
| | | | | (Human) | (Human) | (Human) | (Human) | | GNDSGYFDY |
| | Ab_81C7 | | ND | IGHV2-5 | IGHJ4 | IGLV1-36 | IGU3 | 2195 | AHSMVRGVL |
| | | | | (Human) | (Human) | (Human) | (Human) | | FGADFDY |
| | Ab_81C8 | | ND | IGHV3-33 | IGHJ4 | IGKV1-39 | IGKJ1 | 2196 | ARDGVDFGM |
| | | | | (Human) | (Human) | (Human) | (Human) | | VTLFDY |
| | Ab_81E1 | | ND | IGHV1-24 | IGHJ4 | IGLV1-47 | IGU2 | 2197 | AITSVARGLR |
| | | | | (Human) | (Human) | (Human) | (Human) | | GYFDT |
| | Ab_81E10 | | ND | IGHV1-24 | IGHJ4 | IGLV1-47 | IGU2 | 2198 | AITSLARGLKG |
| | | | | (Human) | (Human) | (Human) | (Human) | | YFDS |
| | Ab_81F2 | | ND | IGHV4-34 | IGHJ5 | IGKV2-28 | IGKJ4 | 2199 | ARGWTVPPL |
| | | | | (Human) | (Human) | (Human) | (Human) | | WVLNWFDP |
| | Ab_82B6 | | ND | IGHV4-61 | IGHJ5 | IGKV2-30 | IGKJ1 | 2200 | AMTYYDYIW |
| | | | | (Human) | (Human) | (Human) | (Human) | | GRVDPQFDP |
| | Ab_82C6 | | ND | IGHV4-39 | IGHJ4 | IGKV2-30 | IGKJ2 | 2201 | ARFITDGYSS |
| | | | | (Human) | (Human) | (Human) | (Human) | | GSDS |
| | Ab_82F6 | | ND | IGHV3-30 | IGHJ4 | IGKV1-33 | IGKJ4 | 2202 | AKQASPYCSG |
| | | | | (Human) | (Human) | (Human) | (Human) | | GSCYSGNFDY |
| | Acharya | | Various | Various | Various | Various | Various | | Various |
| | et al., | | | | | | | | |
| | 2020 | | | | | | | | |
| | B38 | 1206 | DIVMTQSPSFLSASVGDRVTITCRASQ | IGHV3-53 | IGHJ6 | IGKV1-9 | IGKJ2 | 2203 | AREAYGMDV |
| | | | GISSYLAWYQQKPGKAPKLLIYAASTL | (Human) | (Human) | (Human) | (Human) | | |
| | | | QSGVPSRFSGSGSGTEFTLTISSLQPED | | | | | | |
| | | | FATYYCQQLNSYPPYTFGQGTKLEIK | | | | | | |
| | BD-236 | | ND | IGHV3-53 | IGHJ6 | ND | ND | 2204 | ARDLGEAGG |
| | | | | (Human) | (Human) | | | | MDV |
| | BD-368-2 | | ND | ND | ND | ND | ND | | ND |
| | BD-494 | | ND | IGHV3-53 | IGHJ6 | IGKV1-9 | IGKJ3 | 2205 | ARDLVVYGM |
| | | | | (Human) | (Human) | (Human) | (Human) | | DV |
| | BD-498 | | ND | IGHV3-66 | IGHJ6 | IGKV1-9 | IGKJ5 | 2206 | ARDLVVYGM |
| | | | | (Human) | (Human) | (Human) | (Human) | | DV |
| | BD-500 | | ND | IGHV3-53 | IGHJ6 | IGKV1-39 | IGKJ5 | 2207 | ARDAMSYG |
| | | | | (Human) | (Human) | (Human) | (Human) | | MDV |
| | BD-503 | | ND | IGHV3-53 | IGHJ6 | IGKV1-39 | IGKJ3 | 2208 | ARDAAVYGID |
| | | | | (Human) | (Human) | (Human) | (Human) | | V |
| | BD-504 | | ND | IGHV3-66 | IGHJ6 | IGKV1-9 | IGKJ3 | 2209 | ARDLISRGMD |
| | | | | (Human) | (Human) | (Human) | (Human) | | V |
| | BD-505 | | ND | IGHV3-53 | IGHJ6 | IGKV1-33 | IGKJ5 | 2210 | ARDRVVYGM |
| | | | | (Human) | (Human) | (Human) | (Human) | | DV |
| | BD-506 | | ND | IGHV3-53 | IGHJ6 | IGKV1-9 | IGKJ4 | 2211 | ARDLVSYGM |
| | | | | (Human) | (Human) | (Human) | (Human) | | DV |
| | BD-507 | | ND | IGHV3-53 | IGHJ6 | IGKV1-9 | IGKJ3 | 2212 | ARDLVVYGM |
| | | | | (Human) | (Human) | (Human) | (Human) | | DV |
| | BD-508 | | ND | IGHV3-53 | IGHJ6 | IGKV1-39 | IGKJ2 | 2213 | ARDAQNYG |
| | | | | (Human) | (Human) | (Human) | (Human) | | MDV |
| | BD-515 | | ND | IGHV3-66 | IGHJ4 | IGKV1-39 | IGKJ5 | | ND |
| | | | | (Human) | (Human) | (Human) | (Human) | | |
| | BD-604 | | ND | IGHV3-53 | IGHJ6 | IGKV1-9 | IGKJ2 | 2214 | ARDLGPYGM |
| | | | | (Human) | (Human) | (Human) | (Human) | | DV |
| | BD-629 | | ND | IGHV3-53 | IGHJ4 | IGKV3-20 | IGKJ1 | 2215 | ARDYGDYYFD |
| | | | | (Human) | (Human) | (Human) | (Human) | | Y |
| | BD23 | 1207 | DIQMTQSPSTLSASVGDRVTITCRASQ | IGHV7-4- | IGHJ6 | IGKV1-5 | IGKJ2 | 2216 | ARPQGGSSW |
| | | | SISSWLAWYQQKPGKAPKLLIYKASSL | 1 | (Human) | (Human) | (Human) | | YRDYYYGMD |
| | | | ESGVPSRFSGSGSGTEFTLTISSLQPDD | (Human) | | | | | V |
| | | | FATYYCQQYNSYPYTFGQGTKLEIK | | | | | | |
| | Bertoglio | | 17 | Various | Various | Various | Various | | Various |
| | et al., | | | | | | | | |
| | 2020 | | | | | | | | |
| | C002 | 1208 | DIQLTQSPSSLSASVGDRVTITCRASQS | IGHV3-30 | IGHJ4 | IGKV1-39 | IGKJ1 | 2217 | AKEGRPSDIV |
| | | | ISSYLNWYQQKPGKAPKLLIYAASSLQS | (Human) | (Human) | (Human) | (Human) | | VWAFDY |
| | | | GVPSRFSGSGSGTDFTLTISSLQPEDFA | | | | | | |
| | | | TYYCQQSYSTPRTFGQGTKVEIK | | | | | | |
| | C003 | 1209 | EIVLTQSPGTLSLSPGERATLSCRASQS | IGHV3-53 | IGHJ4 | IGKV3-20 | IGKJ2 | 2218 | ARDYGDFYFD |
| | | | VSSTYLAWYQQKPGQAPRLLIYGASSR | (Human) | (Human) | (Human) | (Human) | | Y |
| | | | ATGIPDRFSGSGSGTDFTLTISRLEPED | | | | | | |
| | | | FAVYYCQQYGSSPRTFGQGTKLEIK | | | | | | |
| | C004 | 1210 | AIRMTQSPSSLSASVGDRVTITCQASQ | IGHV1-2 | IGHJ6 | IGKV1-33 | IGKJ5 | 2219 | ASPASRGYSG |
| | | | DISNYLNWYQQKPGKAPKLLIYDASNL | (Human) | (Human) | (Human) | (Human) | | YDHGYYYYM |
| | | | ETGVPSRFSGSGSGTDFTFTISSLQPED | | | | | | DV |
| | | | IATYYCQQYDNLPITFGQGTRLEIK | | | | | | |
| | C005 | 1211 | EIVLTQSPGTLSLSPGERATLSCRASQS | IGHV1-58 | IGHJ3 | IGKV3-20 | IGKJ1 | 2220 | AAPHCSGGSC |
| | | | VRSSYLAWYQQKPGQAPRLLIYGASSR | (Human) | (Human) | (Human) | (Human) | | LDAFDI |
| | | | ATGIPDRFSGSGSGTDFTLTISRLEPED | | | | | | |
| | | | FAVYYCQQYGSSPWTFGQGTKVEIK | | | | | | |
| | C006 | 1212 | QSVLTQPPSASGTPGQRVTVSCSGSSS | IGHV3-11 | IGHJ6 | IGLV1-44 | IGU3 | 2221 | ARRGDGSSSI |
| | | | NIGSNTVNWYQQLPGTAPKLLIYSNN | (Human) | (Human) | (Human) | (Human) | | YYYNYMDV |
| | | | QRPSGVPDRFSGSKSGTSASLAISGLQ | | | | | | |
| | | | SEDEADYFCAAWDDSLNGPVFGGGT | | | | | | |
| | | | KLTVL | | | | | | |
| | C008 | 1213 | DIQMTQSPSTLSASVGDRVTITCRANQ | IGHV3-30 | IGHJ4 | IGKV1-5 | IGKJ1 | 2222 | AREFGDPEW |
| | | | SISSWLAWYQQKPGKAPKLLIYKASSL | (Human) | (Human) | (Human) | (Human) | | YFDY |
| | | | ESGVPSRFSGSGSGTEFTLTISSLQPDD | | | | | | |
| | | | FATYYCQQYNSYWTFGQGTKVEIK | | | | | | |
| | C009 | 1214 | QSALTQPPSASGSPGQSVTISCTGTSS | IGHV1-2 | IGHJ4 | IGLV2-8 | IGU3 | 2223 | ARDSPFSALG |
| | | | DVGGYNYVSWYQQHPGKAPKLMIYE | (Human) | (Human) | (Human) | (Human) | | ASNDY |
| | | | VSKRPSGVPDRFSGSKSGNTASLTVSG | | | | | | |
| | | | LQAEDEAEYYCSSDAGSNNVVFGGGT | | | | | | |
| | | | KLTVL | | | | | | |
| | C010 | 1215 | DIQLTQSPSSLSASVGDRVTITCRASQS | IGHV3-30 | IGHJ4 | IGKV1-39 | IGKJ1 | 2224 | ARDGIVDTAL |
| | | | ISTYLNWYQQKPGKAPKLLIYAASSLQS | (Human) | (Human) | (Human) | (Human) | | VTWFDY |
| | | | GVPSRFSGSGSGTDFTLTISSLQPEDFA | | | | | | |
| | | | TYYCQQSYSTPPWTFGQGTKVEIK | | | | | | |
| | C013 | 1216 | EIVLTQSPATLSLSPGERATLSCRASQS | IGHV1-69 | IGHJ6 | IGKV3-11 | IGKJ4 | 2225 | ARGNRLLYCS |
| | | | VSSYLAWYQQKPGQAPRLLIYDASNR | (Human) | (Human) | (Human) | (Human) | | STSCYLDAVR |
| | | | ATGIPARFSGSGSGTDFTLTISSLEPED | | | | | | QGYYYYYYM |
| | | | FAVYYCQQRSNWPLTFGGGTKVEIK | | | | | | DV |
| | C016 | 1217 | AIRMTQSPSSLSASVGDRVTITCQASQ | IGHV3-30 | IGHJ4 | IGKV1-33 | IGKJ4 | 2226 | AKVTAPYCSG |
| | | | DISNYLNWYQQKPGKAPKLLIYDASNL | (Human) | (Human) | (Human) | (Human) | | GSCYGGNFDY |
| | | | ETGVPSRFSGSGSGTDFTFTINSLQPED | | | | | | |
| | | | IATYYCQQYDNLPPTFGGGTKVEIK | | | | | | |
| | C017 | 1218 | EIVLTQSPATLSLSPGERATLSCRASQS | IGHV3-9 | IGHJ4 | IGKV3-11 | IGKJ5 | 2227 | AKAGVRGIAA |
| | | | VSSYLAWYQQKPGQAPRLLIYDASNR | (Human) | (Human) | (Human) | (Human) | | AGPDLNFDH |
| | | | ATGIPARFSGSGSGTDFTLTISSLEPED | | | | | | |
| | | | FAVYYCQQRITFGQGTRLEIK | | | | | | |
| | C018 | 1219 | DIQLTQSPSSLSASVGDRVTITCRASQS | IGHV3-30 | IGHJ4 | IGKV1-39 | IGKJ2 | 2228 | ARDFDDSSF |
| | | | IRSYLNWYQQKPGKAPKLLIYAASSLQ | (Human) | (Human) | (Human) | (Human) | | WAFDY |
| | | | SGVPSRFSGSGSGTDFTLTISSLQPDDF | | | | | | |
| | | | ATYYCQQSYSTPPATFGQGTKLEIK | | | | | | |
| | C019 | 1220 | SYELTQPPSVSVAPGKTARITCGENNI | IGHV1-46 | IGHJ5 | IGLV3-21 | IGU3 | 2229 | ARVPREGTPG |
| | | | GSKSVHWYQQKPGQAPVLVIYYDSDR | (Human) | (Human) | (Human) | (Human) | | FDP |
| | | | PSGIPERFSGSNSGNTATLTINRVEAG | | | | | | |
| | | | DEADYYCQVWDSSSDHVVFGGGTKL | | | | | | |
| | | | TVL | | | | | | |
| | C021 | 1221 | DIVMTQSPLSLPVTPGEPASISCRSSQS | IGHV4-31 | IGHJ4 | IGKV2-28 | IGKJ3 | 2230 | ARVWQYYDS |
| | | | LLHSNGYNYLDWYLQKPGQSPQLLIYL | (Human) | (Human) | (Human) | (Human) | | SGSFDY |
| | | | GSNRASGVPDRFSGSGSGTDFTLKISR | | | | | | |
| | | | VEAEDVGVYYCMQALQTPFTFGPGTK | | | | | | |
| | | | VDIK | | | | | | |
| | C022 | 1222 | DIQMTQSPSTLSASVGDSVTITCRASQ | IGHV4-39 | IGHJ1 | IGKV1-5 | IGKJ2 | 2231 | ARHAAAYYDR |
| | | | SISSWLAWYQQKPGKAPKLLIYKASSL | (Human) | (Human) | (Human) | (Human) | | SGYYFIEYFQH |
| | | | ESGVPSRFSGSGSGTEFTLTISSLQPDD | | | | | | |
| | | | FATYYCQQYNNYRYTFGQGTKLEIK | | | | | | |
| | C027 | 1223 | DIQMTQSPSTLSASVGDRVTITCRASQ | IGHV3-30 | IGHJ4 | IGKV1-5 | IGKJ1 | 2232 | AKASGIYCSG |
| | | | SISSWLAWYQQKPGKAPKLLIYKASSL | (Human) | (Human) | (Human) | (Human) | | GDCYSYYFDY |
| | | | ESGVPSRFSGSGSGTEFTLTISSLQPDD | | | | | | |
| | | | FATYYCQQYNSYSTFGQGTKVEIK | | | | | | |
| | C029 | 1224 | DIVMTQSPLSLPVTPGEPASISCRSSQS | IGHV4-31 | IGHJ4 | IGKV2-28 | IGKJ4 | 2233 | ARTMYYYDSS |
| | | | LLHSNGYNYLDWYLQKPGQSPQLLIYL | (Human) | (Human) | (Human) | (Human) | | GSFDY |
| | | | GSNRASGVPDRFSGSGSGTDFTLKISR | | | | | | |
| | | | VEAEDVGVYYCMQALQTPHTFGGGT | | | | | | |
| | | | KVEIK | | | | | | |
| | C030 | 1225 | DIQMTQSPSTLSASVGDRVTITCRASQ | IGHV3-30 | IGHJ4 | IGKV1-5 | IGKJ1 | 2234 | AKASGIYCSG |
| | | | SISSWLAWYQQKPGKAPKLLIYKASSL | (Human) | (Human) | (Human) | (Human) | | GNCYSYYFDY |
| | | | ESGVPSRFSGSGSGTEFTLTISSLQPDD | | | | | | |
| | | | FATYYCQQYNSYSTFGQGTKVEIK | | | | | | |
| | C031 | 1226 | DIQMTQSPSSLSASVGDRVTITCRASQ | IGHV3-13 | IGHJ2 | IGKV1-39 | IGKJ4 | 2235 | ARVGYDSSGY |
| | | | SISSYLNWYQQKPGKAPKVLIYAASSL | (Human) | (Human) | (Human) | (Human) | | SGWYFDL |
| | | | QSGVPSRFSGSGSGTDFTLTISSLQPED | | | | | | |
| | | | FATYYCQQSYSTPPLTFGGGTKVEIK | | | | | | |
| | C101 | 1227 | EIVLTQSPGTLSLSPGERATLSCRASQS | IGHV3-53 | IGHJ4 | IGKV3-20 | IGKJ1 | 2236 | VRDYGDFYFD |
| | | | VSSSYLAWYQQKPGQAPRLLIYGASSR | (Human) | (Human) | (Human) | (Human) | | Y |
| | | | ATGIPDRFSGGGSETDFTLTISRLEPED | | | | | | |
| | | | CAVYYCQQYGSSPRTFGQGTKVEIK | | | | | | |
| | C102 | 1228 | EIVLTQSPGTLSLSPGERATLSCRASQS | IGHV3-53 | IGHJ4 | IGKV3-20 | IGKJ1 | 2237 | ARDYGDYYFD |
| | | | VSSSYLAWYQQKPGQAPRLLIYGASSR | (Human) | (Human) | (Human) | (Human) | | Y |
| | | | ATGIPDRFSGSGSGTDFTLTISRLEPED | | | | | | |
| | | | FAVYYCQQYGSSPRTFGQGTKVEIK | | | | | | |
| | C103 | 1229 | EIVLTQSPGTLSLSPGERATLSCRASQT | IGHV4-34 | IGHJ3 | IGKV3-20 | IGKJ4 | 2238 | ARKPLLYSDFS |
| | | | VTANYLAWYQQKPGQAPRLLIYGASK | (Human) | (Human) | (Human) | (Human) | | PGAFDI |
| | | | RATGIPDRFSGSGSGTDFTLSISRLEPE | | | | | | |
| | | | DFAVYYCQQYTTTPRTFGGGTKVEIK | | | | | | |
| | C104 | 1230 | EIVLTQSPGTVSLSPGERATLSCWASQ | IGHV4-34 | IGHJ3 | IGKV3-20 | IGKJ4 | 2239 | ARKPLLYSNLS |
| | | | SVSASYLAWYQQKPGQAPRLLIYGASS | (Human) | (Human) | (Human) | (Human) | | PGAFDI |
| | | | RATGIPDRFSGSGSGTDFTLTISRLEPE | | | | | | |
| | | | DFAVYYCQQYGTTPRTFGGGTKVEIK | | | | | | |
| | C105 | 1231 | QSALTQPPSASGSPGQSVTISCTGTSS | IGHV3-53 | IGHJ4 | IGLV2-8 | IGU3 | 2240 | ARGEGWELP |
| | | | DVGGYKYVSWYQQHPGKAPKLMIYE | (Human) | (Human) | (Human) | (Human) | | YDY |
| | | | VSKRPSGVPDRFSGSKSGNTASLTVSG | | | | | | |
| | | | LQAEDEADYYCSSYEGSNNFVVFGGG | | | | | | |
| | | | TKLTVL | | | | | | |
| | C106 | 1232 | SYELTQPPSVSVAPGKTARITCGGNNI | IGHV4-61 | IGHJ4 | IGLV3-21 | IGU3 | 2241 | ARERPGGTYS |
| | | | GSKSVHWYQQKPGQAPVLVIYFDSDR | (Human) | (Human) | (Human) | (Human) | | NTWYTPTDT |
| | | | PSGIPERFSGSNSGNTATLTISRVEAGD | | | | | | NWFDT |
| | | | EADYYCQVWDSSRDHVVFGGGTKLT | | | | | | |
| | | | VL | | | | | | |
| | C107 | 1233 | QSVLTQPPSASGTPGQRVTISCSGSSS | IGHV1-18 | IGHJ4 | IGLV1-47 | IGU3 | 2242 | ARGEAVAGTT |
| | | | NIGSNYVYWYQQLPGTAPKLLIYRNN | (Human) | (Human) | (Human) | (Human) | | GFFDY |
| | | | QRPSGVPDRFSGSKSGTSASLAISGLRS | | | | | | |
| | | | EDEADYYCAAWDDSLSGFVVFGGGTK | | | | | | |
| | | | LTVL | | | | | | |
| | C108 | 1234 | QSALTQPASVSGSPGQSITISCTGTSSD | IGHV4-4 | IGHJ3 | IGLV2-14 | IGU1 | 2243 | VRDGGRPGD |
| | | | VGGYNYVSWYQQHPGKAPKLMIYDV | (Human) | (Human) | (Human) | (Human) | | AFDI |
| | | | SNRPSGVSNRFSGSKSGNTASLTISGL | | | | | | |
| | | | QAEDEADYYCNSYTSSSTRVFGTGTKV | | | | | | |
| | | | TVL | | | | | | |
| | C109 | 1235 | QSALTQPPSASGSPGQSVTISCTGTSS | IGHV3-7 | IGHJ4 | IGLV2-8 | IGU3 | 2244 | AIQLWLRGGY |
| | | | DVGGYNYVSWYQQHPGKAPKLMIYE | (Human) | (Human) | (Human) | (Human) | | DY |
| | | | VTKRPSGVPDRFSGSKSGNTASLTVSG | | | | | | |
| | | | LQAEDEADYYCSSYAGSNNYVVFGGG | | | | | | |
| | | | TKLTVL | | | | | | |
| | C110 | 1236 | DIQMTQSPSTLSASVGDRVTITCRASQ | IGHV5-51 | IGHJ3 | IGKV1-5 | IGKJ2 | 2245 | ARSFRDDPRI |
| | | | SISYWLAWYQQKPGKAPKLLIYQASSL | (Human) | (Human) | (Human) | (Human) | | AVAGPADAF |
| | | | ESGVPSRFSGSESGTEFTLTISSLQPDD | | | | | | DI |
| | | | FATYYCQQYNSYPYTFGQGTKLEIK | | | | | | |
| | C111 | 1237 | QSVLTQPPSVSEAPRQRVTISCSGSSS | IGHV4-59 | IGHJ3 | IGLV1-36 | IGU3 | 2246 | ARVEDWGYC |
| | | | NIGNNAVNWYQQVPGKAPKLLIYYDD | (Human) | (Human) | (Human) | (Human) | | SSTNCYSGAF |
| | | | LLPSGVSDRFSGSKSGTSASLAISGLQS | | | | | | DI |
| | | | EDEADYYCAAWDDSLNGAWVFGGG | | | | | | |
| | | | TKLTVL | | | | | | |
| | C112 | 1238 | QSALTQPASVSGSPGQSITISCTGTSSD | IGHV3-30 | IGHJ4 | IGLV2-14 | IGU3 | 2247 | AREDYYDSSG |
| | | | VGGYNYVSWYQQHPGKAPKLMIYDV | (Human) | (Human) | (Human) | (Human) | | SFDY |
| | | | SNRPSGVSNRFSGSKSGNTASLTISGL | | | | | | |
| | | | QAEDEADYYCSSYTSSSTWVFGGGTKL | | | | | | |
| | | | TVL | | | | | | |
| | C113 | 1239 | DIQMTQSPSTLSASVGDRVTITCRASQ | IGHV3-33 | IGHJ3 | IGKV1-5 | IGKJ4 | 2248 | ARGVNPDDIL |
| | | | SMSSWLAWYQQKPGNAPKLLIYKASS | (Human) | (Human) | (Human) | (Human) | | TGVDAFDI |
| | | | LESGVPSRFSGSGSGTEFTLTISSLQPD | | | | | | |
| | | | DFATYYCQQHNSSPLTFGGGTKVEIK | | | | | | |
| | C114 | 1240 | QSVLTQPPSVSGAPGQRVTISCTGTSS | IGHV3-53 | IGHJ4 | IGLV1-40 | IGU3 | 2249 | ARGDGELFFD |
| | | | NIGAGYDVHWYQQLPGRAPKVLISGN | (Human) | (Human) | (Human) | (Human) | | Q |
| | | | NIRPSEVPDRFSGSRSGTSASLAITSLQ | | | | | | |
| | | | PEDEAQYYCQSYDSSLYAVFGGGTKLT | | | | | | |
| | | | VL | | | | | | |
| | C115 | 1241 | DIVMTQSPLSLSVTPGEPASISCRSSQS | IGHV3-49 | IGHJ4 | IGKV2-28 | IGKJ2 | 2250 | TRWDGWSQ |
| | | | LLHSNGNNYFDWYLQKPGQSPQLLIY | (Human) | (Human) | (Human) | (Human) | | HDY |
| | | | LGSNRASGVPDRFSGSGSGTDFTLKIS | | | | | | |
| | | | RVEAEDVGVYYCMQVLQIPYTFGQGT | | | | | | |
| | | | KLEIK | | | | | | |
| | C116 | 1242 | NFMLTQPHSVSESPGKTVTISCTGSSG | IGHV3-30 | IGHJ5 | IGLV6-57 | IGU3 | 2251 | ARDFYHNWF |
| | | | SIASNYVQWYQQRPGSAPTTVIYEDN | (Human) | (Human) | (Human) | (Human) | | DP |
| | | | QRPSGVPDRFSGSIDRSSNSASLTISGL | | | | | | |
| | | | KTEDEADYYCQSYDSGNHWVVFGGG | | | | | | |
| | | | TRLTVL | | | | | | |
| | C117 | 1243 | QSVLTQPPSVSAAPGQKVTISCSGSSS | IGHV3-30 | IGHJ4 | IGLV1-51 | IGU1 | 2252 | ARDPIWFGEL |
| | | | NIGNNLVSWYQQLPGTAPKLLIYENN | (Human) | (Human) | (Human) | (Human) | | LSPPFVHFDY |
| | | | KRPSGIPDRFSGSKSGTSATLGITGLQT | | | | | | |
| | | | GDEADYYCGAWDSSLSAGGVYVFGT | | | | | | |
| | | | GTKVTVL | | | | | | |
| | C118 | 1244 | QPVLTQSPSASASLGASVKLTCTLSSGH | IGHV3-30 | IGHJ6 | IGLV4-69 | IGU3 | 2253 | ASGYTGYDYF |
| | | | SSYAIAWHQQQPEKGPRYLMKLNTD | (Human) | (Human) | (Human) | (Human) | | VRGDYYGLD |
| | | | GSHSKGDGIPDRFSGSSSGAERYLTISS | | | | | | V |
| | | | LQSEDEADYYCQTWGTGILVFGGGTK | | | | | | |
| | | | LTVL | | | | | | |
| | C119 | 1245 | QSALTQPASVSGSPGQSITISCTGTSSD | IGHV1-46 | IGHJ6 | IGLV2-14 | IGU3 | 2254 | ARANHETTM |
| | | | VGGYKYVSWYQRHPGKAPKLMIYDV | (Human) | (Human) | (Human) | (Human) | | DTYYYYYYMD |
| | | | SNRPSGVSNRFSGSKSGNTASLTISGL | | | | | | V |
| | | | QAEDEADYYCSSYTSSSTSVVFGGGTQ | | | | | | |
| | | | LTVL | | | | | | |
| | C120 | 1246 | AIRMTQSPSSLSASVGDTVTITCQASQ | IGHV3-53 | IGHJ4 | IGKV1-33 | IGKJ4 | 2255 | AREGMGMA |
| | | | DISKYLNWYQQKPGKAPKLLIYDASNL | (Human) | (Human) | (Human) | (Human) | | AAGT |
| | | | ETGVPSRFSGSGSGTDFTFTISSLQPED | | | | | | |
| | | | IATYYCQQYDNLPQTFGGGTKVEIK | | | | | | |
| | C121 | 1247 | QSALTQPASVSGSPGQSITISCTGTSSD | IGHV1-2 | IGHJ6 | IGLV2-23 | IGU3 | 2256 | ARAPLFPTGV |
| | | | VGSYNLVSWYQQHPGKAPKLMIYEGS | (Human) | (Human) | (Human) | (Human) | | LAGDYYYYG |
| | | | KRPSGVSNRFSGSKSGNTASLTISGLQ | | | | | | MDV |
| | | | AEDEADYYCCSYAGSSTLVFGGGTKLT | | | | | | |
| | | | VL | | | | | | |
| | C122 | 1248 | DIQLTQSPSFLSASVGDRVTITCRASQG | IGHV3-53 | IGHJ4 | IGKV1-9 | IGKJ2 | 2257 | ARESGDTTM |
| | | | ISSYLAWYQQKPGKAPKLLIYAASTLQS | (Human) | (Human) | (Human) | (Human) | | AFDY |
| | | | GVPSRFSGSGSGTEFTLTISSLQPEDFA | | | | | | |
| | | | TYYCQQLNSDSYTFGQGTKLEIK | | | | | | |
| | C123 | 1249 | DIQLTQSPSFLSASVGDRVTITCRASQG | IGHV3-53 | IGHJ3 | IGKV1-9 | IGKJ5 | 2258 | ARDLSAAFDI |
| | | | ISSYLAWYQQKPGKAPKLLIYAASTLQS | (Human) | (Human) | (Human) | (Human) | | |
| | | | GVPSRFSGSGSGTEFTLTISSLQPEDFA | | | | | | |
| | | | TYYCQQLNSYPPAFGQGTRLEIK | | | | | | |
| | C124 | 1250 | EIVLTQSPATLSLSPGERATLSCRASQS | IGHV3-48 | IGHJ4 | IGKV3-11 | IGKJ1 | 2259 | AREGARVGA |
| | | | FSSYLAWYQQKPGQAPRLLIYDASNRA | (Human) | (Human) | (Human) | (Human) | | TYDTYYFDY |
| | | | TGIPARFSGSGSGTDFTLTISSLEPEDF | | | | | | |
| | | | AVYYCQQRNNWPPEWTFGQGTKVEIK | | | | | | |
| | C125 | 1251 | EIVLTQSPGTLSLSPGERATLSCRASQS | IGHV1-58 | IGHJ3 | IGKV3-20 | IGKJ1 | 2260 | AAPYCSGGSC |
| | | | VSSSYLAWYQQKPGQAPRLLIYGASSR | (Human) | (Human) | (Human) | (Human) | | SDAFDI |
| | | | ATGIPDRFSGSGSGTDFTLTISRLEPED | | | | | | |
| | | | FAVYYCQQYGSSPWTFGQGTKVEIK | | | | | | |
| | C126 | 1252 | NFMLTQPHSVSESPGKTVTISCTGSSG | IGHV4-59 | IGHJ3 | IGLV6-57 | IGU3 | 2261 | ARLQWLRGA |
| | | | SIASNYVQWYQQRPGSAPTTVINEDN | (Human) | (Human) | (Human) | (Human) | | FDI |
| | | | QRPSGVPDRFSGSIDSSSNSASLTISGL | | | | | | |
| | | | KTEDEADYYCQSYDSSNLVFGGGTKLT | | | | | | |
| | | | VL | | | | | | |
| | C127 | 1253 | QSVLTQPPSASGTPGQRVTISCSGSSS | IGHV1-2 | IGHJ6 | IGLV1-44 | IGU3 | 2262 | ATAHPRRIQG |
| | | | NIGSNTVNWYQQLPGTAPKLLIYSNN | (Human) | (Human) | (Human) | (Human) | | VFFLGPGV |
| | | | QRPSGVPDRFSGSKSGTSASLAISGLQ | | | | | | |
| | | | SEDEADYYCAAWDDSLNGVVFGGGT | | | | | | |
| | | | KLTVL | | | | | | |
| | C128 | 1254 | EIVLTQSPGTLSLSPGERATLSCRASQS | IGHV3-23 | IGHJ6 | IGKV3-20 | IGKJ4 | 2263 | ANHPLASGD |
| | | | VNSRQLAWYQQKPGQAPRLLIYGASS | (Human) | (Human) | (Human) | (Human) | | DYYHYYMDV |
| | | | RATGIPERFSGSGSGTDFTLTISRLESE | | | | | | |
| | | | DFAVYHCQQYGSSRALTFGGGTKVEIK | | | | | | |
| | C130 | 1255 | SYELTQPPSVSVAPGKTARITCGGNNI | IGHV1-46 | IGHJ2 | IGLV3-21 | IGU3 | 2264 | ARSRPTPDW |
| | | | GSKSVHWYQQKPGQAPVLVIYYDSDR | (Human) | (Human) | (Human) | (Human) | | YFDL |
| | | | PSGIPERFSGSNSGNTATLTISRVEAGD | | | | | | |
| | | | EADYYCQVWDSSSDHPGVVFGGGTK | | | | | | |
| | | | LTVL | | | | | | |
| | C131 | 1256 | EIVMTQSPATLSVSPGERATLSCRASQ | IGHV1-69 | IGHJ6 | IGKV3-15 | IGKJ5 | 2265 | ARVNQAVTT |
| | | | SVSSNLAWYQQKPGQAPRLLIYGAST | (Human) | (Human) | (Human) | (Human) | | PFSMDV |
| | | | RATGIPARFSGSGSGTEFTLTISSLQSE | | | | | | |
| | | | DFAVYYCQQYNNWPITFGQGTRLEIK | | | | | | |
| | C132 | 1257 | QSALTQPASVSGSPGQSITISCTGTSSD | IGHV4-4 | IGHJ4 | IGLV2-14 | IGU3 | 2266 | ARGGDTAMG |
| | | | VGGYNYVSWYQQHPGKAPKLMIYDV | (Human) | (Human) | (Human) | (Human) | | PEYFDY |
| | | | SNRPSGVSNRFSGSKSGNTASLTISGL | | | | | | |
| | | | QAEDEADYYCSSYTSSSTLLFGGGTKLT | | | | | | |
| | | | VL | | | | | | |
| | C133 | 1258 | DIQMTQSPSSLSASVGDRVTITCRASQ | IGHV3-30 | IGHJ4 | IGKV1-39 | IGKJ1 | 2267 | ARDSDVDTS |
| | | | SISSYLNWYQQKPGKAPKLLIYAASSLQ | (Human) | (Human) | (Human) | (Human) | | MVTWFDY |
| | | | SGVPSRFSGSGSGTDFTLTISSLQPEDF | | | | | | |
| | | | ATYYCQQSYSTPPWTFGQGTKVEIK | | | | | | |
| | C134 | 1259 | SYELTQPPSVSVAPGKTARITCGGNNI | IGHV3-23 | IGHJ4 | IGLV3-21 | IGU3 | 2268 | AKDPLITGPTY |
| | | | GSKSVHWYQQKPGQAPVLVIYYDSDR | (Human) | (Human) | (Human) | (Human) | | QYFHY |
| | | | PSGIPERFSGSNSGNTATLTISRVEAGD | | | | | | |
| | | | EAEYHCQVWDSSSDRPGVVFGGGTK | | | | | | |
| | | | LTVL | | | | | | |
| | C135 | 1260 | DIQMTQSPSTLSASVGDRVTITCRASQ | IGHV3-30 | IGHJ4 | IGKV1-5 | IGKJ1 | 2269 | ASSSGYLFHS |
| | | | SISNWLAWFQQKPGKAPKLLIYEASSL | (Human) | (Human) | (Human) | (Human) | | DY |
| | | | ESGVPSRFSGSGSGTEFTLTISSLQPDD | | | | | | |
| | | | FATYYCQQYNSYPWTFGQGTKVEIK | | | | | | |
| | C138 | 1261 | NFMLTQPHSVSESPGKTVTISCTGSSG | IGHV3-7 | IGHJ4 | IGLV6-57 | IGU3 | 2270 | AGGTWLRSSF |
| | | | SIASNYVQWYQQRPGSAPTTVIYEDN | (Human) | (Human) | (Human) | (Human) | | DY |
| | | | QRPSGVPDRFSGSIDSSSNSASLTISGL | | | | | | |
| | | | KTEDEADYYCQSYDSSNWVFGGGTKL | | | | | | |
| | | | TVL | | | | | | |
| | C139 | 1262 | DIQMTQSPSSLSASVGDRVTITCQASQ | IGHV3- | IGHJ6 | IGKV1-33 | IGKJ4 | 2271 | AKGGAYSYYY |
| | | | DISNYLNWYQQKPGKAPKLLIYDASNL | 303 | (Human) | (Human) | (Human) | | YMDV |
| | | | ETGVPSRFSGSGSGTDFTFTISSLQPED | (Human) | | | | | |
| | | | IATYYCQQYDNLPLTFGGGTKVEIK | | | | | | |
| | C140 | 1263 | DIQLTQSPSFLSASVGDRVTITCRASQG | IGHV3-66 | IGHJ6 | IGKV1-9 | IGKJ2 | 2272 | ARDLYYYGM |
| | | | ISSYLAWYQQKPGKAPKLLIYAASTLQS | (Human) | (Human) | (Human) | (Human) | | DV |
| | | | GVPSRFSGSGSGTEFTLTISSLQPEDFA | | | | | | |
| | | | TYYCQQLNSYSYTFGQGTKLEIK | | | | | | |
| | C141 | 1264 | NFMLTQPHSVSESPGKTVTISCTGSSG | IGHV3-30 | IGHJ4 | IGLV6-57 | IGU3 | 2273 | ARADLGYCTN |
| | | | SIASNYVQWYQQRPGSAPTTVIYEDN | (Human) | (Human) | (Human) | (Human) | | GVCYVDY |
| | | | QRPSGVPDRFSGSIDSSSNSASLTISGL | | | | | | |
| | | | KTEDEADYYCQSYDSSNWVFGGGTKL | | | | | | |
| | | | TVL | | | | | | |
| | C143 | 1265 | QSALTQPASVSGSPGQSITISCTGTSND | IGHV3-66 | IGHJ3 | IGLV2-23 | IGU3 | 2274 | ARDSSEVRDH |
| | | | VGSYTLVSWYQQYPGKAPKLLIFEGTK | (Human) | (Human) | (Human) | (Human) | | PGHPGRSVG |
| | | | RSSGISNRFSGSKSGNTASLTISGLQGE | | | | | | AFDI |
| | | | DEADYYCCSYAGASTFVFGGGTKLTVL | | | | | | |
| | C144 | 1266 | QSALTQPASVSGSPGQSITISCTGTSSD | IGHV3-53 | IGHJ4 | IGLV2-14 | IGU1 | 2275 | AREGEVEGY |
| | | | VGGYNYVSWYQQHPGKAPKLMIYDV | (Human) | (Human) | (Human) | (Human) | | NDFWSGYSR |
| | | | SNRPSGVSNRFSGSKSGNTASLTISGL | | | | | | DRYYFDY |
| | | | QAEDEADYYCSSYTSSSTRVFGTGTKV | | | | | | |
| | | | TVL | | | | | | |
| | C145 | 1267 | QSALTQPASVSGSPGQSITISCTGTSSD | IGHV3-53 | IGHJ4 | IGLV2-14 | IGU1 | 2276 | AREGEVEGYY |
| | | | VGGYNYVSWYQQHPGKAPKLMIYDV | (Human) | (Human) | (Human) | (Human) | | DFWSGYSRD |
| | | | SNRPSGVSNRFSGSKSGNTASLTISGL | | | | | | RYYFDY |
| | | | QAEDEADYYCSSYTSSTTRVFGTGTRV | | | | | | |
| | | | TVL | | | | | | |
| | C146 | 1268 | QSALTQPASVSGSPGQSITISCTGTSSD | IGHV3-21 | IGHJ4 | IGLV2-14 | IGU1 | 2277 | ARDVASNYAY |
| | | | IGVYNYISWSQQHPGKAPKVMIYDVT | (Human) | (Human) | (Human) | (Human) | | FDL |
| | | | NRPSGVSNRFSGSKSGNTASLTISGLQ | | | | | | |
| | | | AEDEADYYCSSYRGSSTPYVFGTGTKV | | | | | | |
| | | | TVL | | | | | | |
| | C147 | 1269 | QAVVTQEPSLTVSPGGTVTLTCGSSTG | IGHV5-51 | IGHJ5 | IGLV7-46 | IGU3 | 2278 | ARLSDRWYSP |
| | | | AVTSGHYPYWFQQKSGQAPRTLIYETS | (Human) | (Human) | (Human) | (Human) | | FDP |
| | | | IKHSWTPARFSGSLLGGKAALTLSGAQ | | | | | | |
| | | | PEDEADYYCLLSYSGARPVFGGGTKLT | | | | | | |
| | | | VL | | | | | | |
| | C148 | 1270 | EIVMTQSPATLSVSPGERATLSCRASQ | IGHV3-66 | IGHJ6 | IGKV3-15 | IGKJ4 | 2279 | ARIANYMDV |
| | | | SVSSHLAWYQQKPGQAPRLLIYGAST | (Human) | (Human) | (Human) | (Human) | | |
| | | | RATGIPTRFSGSGSGTEFTLTISSLQSE | | | | | | |
| | | | DFAVYYCQQYNNWPPLTFGGGTKVEIK | | | | | | |
| | C150 | 1271 | QSALTQPASVSGSPGQSITISCTGTSSD | IGHV3-74 | IGHJ6 | IGLV2-14 | IGU3 | 2280 | ARPTAVAAA |
| | | | VGYYNFVSWYQQHPGKAPKLMIYEVS | (Human) | (Human) | (Human) | (Human) | | GNYFYYYGM |
| | | | NRPSGVSNRFSGSKSGNTASLIISGLQA | | | | | | DV |
| | | | EDEADYYCSSYRSSSTLVFGGGTKLTVL | | | | | | |
| | C151 | 1272 | NFMLTQPHSVSESPGKTVTISCTGSSG | IGHV3-21 | IGHJ4 | IGLV6-57 | IGU3 | 2281 | ARERGYDGG |
| | | | SIASNYVQWYQQRPGSAPTTVIYEDN | (Human) | (Human) | (Human) | (Human) | | KTPP |
| | | | QRPSGVPDRFSGSIDSSSNSASLTISGL | | | | | | |
| | | | KTEDEADYYCQSYDSSNYWVFGGGTK | | | | | | |
| | | | LTVL | | | | | | |
| | C153 | 1273 | QSALTQPASVSGSPGQSITISCTGTSSD | IGHV3-53 | IGHJ4 | IGLV2-23 | IGU3 | 2282 | ARVGGAHSG |
| | | | VGSYNLVSWYQQHPGKAPKLMIYEGS | (Human) | (Human) | (Human) | (Human) | | YDGSFDY |
| | | | KRPSGVSNRFSGSKSGNTASLTISGLQ | | | | | | |
| | | | AEDEADYYCCSYAGSSTWVFGGGTKL | | | | | | |
| | | | TVL | | | | | | |
| | C154 | 1274 | DIQMTQSPSSLSASVGDRVTITCQASQ | IGHV3-30 | IGHJ4 | IGKV1-33 | IGKJ5 | 2283 | AKQAGPYCS |
| | | | GISNYLNWYQQKPGKAPKLLIYDASNL | (Human) | (Human) | (Human) | (Human) | | GGSCYSAPFD |
| | | | ETGVPSRFSGSGSGTDFTFTISSLQPED | | | | | | Y |
| | | | IATYYCQQYDNLPITFGQGTRLEIK | | | | | | |
| | C155 | 1275 | EIVMTQSPATLSVSPGERATLSCRASQ | IGHV3-53 | IGHJ4 | IGKV3-15 | IGKJ1 | 2284 | ARDFGEFYFD |
| | | | SVSSNLAWYQQKPGQAPRLLIYGAST | (Human) | (Human) | (Human) | (Human) | | Y |
| | | | RATAIPARFSGSGSGTEFTLTISSLQSE | | | | | | |
| | | | DFAVYYCQQYNNWPRTFGQGTKVEIK | | | | | | |
| | C156 | 1276 | SYELTQPPSVSVAPGQTARISCGGNNI | IGHV3-30 | IGHJ4 | IGLV3-21 | IGU3 | 2285 | AKDPFPLAVA |
| | | | GSKNVHWYQQKPGQAPVLVVYDDS | (Human) | (Human) | (Human) | (Human) | | GTGYFDY |
| | | | DRPSGIPERFSGSNSGNTATLTISRVEA | | | | | | |
| | | | GDEADYYCQVWDSSSDPWVFGGGTK | | | | | | |
| | | | LTVL | | | | | | |
| | C164 | 1277 | QSALTQPASVSGSPGQSITISCTGTSSD | IGHV3-66 | IGHJ3 | IGLV2-23 | IGU3 | 2286 | ARDSSEVRDH |
| | | | VGSYTLVSWYQQYPGKAPKLLIFEVTK | (Human) | (Human) | (Human) | (Human) | | PGHPGRSVG |
| | | | RSSGISNRFSGSKSGNTASLTISGLQGE | | | | | | AFDI |
| | | | DEADYYCCSYAGASTFVFGGGTKLTVL | | | | | | |
| | C165 | 1278 | EIVLTQSPGTLSLSPGERATLSCRASQS | IGHV1-69 | IGHJ3 | IGKV3-20 | IGKJ1 | 2287 | ARDLLDPQLD |
| | | | VSSTYLAWYQQKPGQAPRLLIYGASSR | (Human) | (Human) | (Human) | (Human) | | DAFDI |
| | | | ATGIPDRFSGSGSGTDFTLTISRLEPED | | | | | | |
| | | | FAVYYCQQYGSSPWTFGQGTKVEIK | | | | | | |
| | C201 | 1279 | IRMTQSPSSVSASVGDRVTITCRASQG | IGHV3-9 | IGHJ4 | IGKV1-12 | IGKJ4 | 2288 | VKGVEYSSSS |
| | | | ISSWLAWYQQKPGKAPKLLIYVESSLQ | (Human) | (Human) | (Human) | (Human) | | NFDY |
| | | | SGVPSRFSGSGSGTDFTLTISSLQPEDF | | | | | | |
| | | | ATYYCQQANSFPLTFGGGTKVEIK | | | | | | |
| | C202 | 1280 | DIQLTQSPSSLSASVGDRVTITCQASQ | IGHV3-66 | IGHJ4 | IGKV1-33 | IGKJ2 | 2289 | ARDTLGRGG |
| | | | DISNYLNWYQQKPGKAPKLLIYDASNL | (Human) | (Human) | (Human) | (Human) | | DY |
| | | | ETGVPSRFSGSGSGTDFTFTISSLQPED | | | | | | |
| | | | IATYYCQQYDNLPRSFGQGTKLEIK | | | | | | |
| | C204 | 1281 | DIQLTQSPSSLSASVGDRVTITCRASQS | IGHV3-23 | IGHJ5 | IGKV1-39 | IGKJ1 | 2290 | ARESDCGSTS |
| | | | ISSYLNWYQQKPGKAPKLLIYAASSLQS | (Human) | (Human) | (Human) | (Human) | | CYQVGWFDP |
| | | | GVPSRFSGSGSGTDFTLTISSLQPEDFA | | | | | | |
| | | | TYYCQQSYSTPPWTFGQGTKVEIK | | | | | | |
| | C205 | 1282 | EIVLTQSPGTLSLSPGERATLSCRASQS | IGHV1-46 | IGHJ4 | IGKV3-20 | IGKJ1 | 2291 | ARGPERGIVG |
| | | | VSSSYLAWYQQKPGQAPRLLIYGASSR | (Human) | (Human) | (Human) | (Human) | | ATDYFDY |
| | | | ATGIPDRFSGSGSGTDFTLTISRLEPED | | | | | | |
| | | | FAVYYCQQYVSSPWTFGQGTKVEIK | | | | | | |
| | C207 | 1283 | EIVLTQSPATLSLSPGERATLSCRASQS | IGHV3-23 | IGHJ4 | IGKV3-11 | IGKJ1 | 2292 | AKEPIGQPLL |
| | | | VSSYLAWYQQKPGQAPRLLIYDASNR | (Human) | (Human) | (Human) | (Human) | | WWDY |
| | | | ATGIPARFSGSGSGTDFTLTISSLEPED | | | | | | |
| | | | FAVYYCQQRSNWPRGFGQGTKVEIK | | | | | | |
| | C208 | 1284 | EIVLTQSPGTLSLSPGERATLSCRASQS | IGHV5-51 | IGHJ5 | IGKV3-20 | IGKJ4 | 2293 | ARGPNLQNW |
| | | | VSGSYLAWYQQRPGQAPRLLIYGASS | (Human) | (Human) | (Human) | (Human) | | FDP |
| | | | RATGIPDRFSGSGSGTDFTLTISRLEPE | | | | | | |
| | | | DFAVYYCQQYGSSLTFGGGTKVEIK | | | | | | |
| | C210 | 1285 | DIQLTQSPSFLSASVGDRVTITCRASQG | IGHV3-53 | IGHJ6 | IGKV1-9 | IGKJ4 | 2294 | ARDLMAYGM |
| | | | ISSYLAWYQQKPGKAPKLLIYAASTLQS | (Human) | (Human) | (Human) | (Human) | | DV |
| | | | GVPSRFSGSGSGTEFTLTISSLQPEDFA | | | | | | |
| | | | TYYCQQLNSYPQGTFGGGTKVEIK | | | | | | |
| | C211 | 1286 | EIVMTQSPATLSVSPGERATLSCRASQ | IGHV3-66 | IGHJ4 | IGKV3-15 | IGKJ1 | 2295 | ARDYGDFYFD |
| | | | SVSSNLAWYQQKPGQGPRLLIYGAST | (Human) | (Human) | (Human) | (Human) | | F |
| | | | RATGIPARFSGSGSGTEFTLTISSLQSE | | | | | | |
| | | | DFAVYYCQQYNNWPRTFGQGTKVEIK | | | | | | |
| | C212 | 1287 | LTQPASVSGSPGQSITISCTGTSSDVGS | IGHV1-2 | IGHJ6 | IGLV2-23 | IGU3 | 2296 | ARERYFDLGG |
| | | | YNLVSWYQQHPGKAPKLMIYEDSKRP | (Human) | (Human) | (Human) | (Human) | | MDV |
| | | | SGVSNRFSGSKSGNTASLTISGLQAED | | | | | | |
| | | | EADYYCCSYAGSSTRLFGGGTKLTVL | | | | | | |
| | C214 | 1288 | DIQLTQSPSSLSASVGDRVTITCRASQS | IGHV3-33 | IGHJ5 | IGKV1-39 | IGKJ1 | 2297 | ARDVGRVTT |
| | | | ISSYLTWYQQKPGKAPKLLIYAASSLQS | (Human) | (Human) | (Human) | (Human) | | WFDP |
| | | | GVPSRFSGSGSGTDFTLTISSLQPEDFA | | | | | | |
| | | | TYYCQQSYSTPPWTFGQGTKVEIK | | | | | | |
| | C215 | 1289 | DIQLTQSPSSLSASVGDRVTITCRASQS | IGHV3-23 | IGHJ5 | IGKV1-39 | IGKJ1 | 2298 | ASEEDYSNYV |
| | | | ISSYLNWYQQKPGKAPKLLIYAASSLQS | (Human) | (Human) | (Human) | (Human) | | GWFDP |
| | | | GVPSRFSGSGSGTDFTLTISSLQPEDFA | | | | | | |
| | | | TYYCQQSYSTPPWTFGQGTKVEIK | | | | | | |
| | C216 | 1290 | DIQLTQSPSSLSASVGDRVTITCRASQS | IGHV3-13 | IGHJ2 | IGKV1-39 | IGKJ5 | 2299 | ARDRGSSGW |
| | | | ISSYLNWYQQKPGKAPKLLIYVASSLQS | (Human) | (Human) | (Human) | (Human) | | YGWYFDL |
| | | | GVPSRFSGSGSGTDFTLTISSLQPEDFA | | | | | | |
| | | | TYYCQQSYSTPPITFGQGTRLEIK | | | | | | |
| | CA1 | 1291 | DIVMTQTPATLSLSPGERATLSCRASQ | IGHV1-18 | IGHJ6 | IGKV3-11 | IGKJ3 | 2300 | AREGYCSGGS |
| | | | SVSSYLAWYQQKPGQAPRLLIYDASN | (Human) | (Human) | (Human) | (Human) | | CYSGYYYYYG |
| | | | RATGIPARFSGSGSGTDFTLTISSLEPE | | | | | | MDV |
| | | | DFAVYYCQQRRNWGTFGPGTKVDIK | | | | | | |
| | CB6 | 1292 | DIVMTQSPSSLSASVGDRVTITCRASQ | IGHV3-66 | IGHJ4 | IGKV1-39 | IGKJ2 | 2301 | ARVLPMYGD |
| | | | SISRYLNWYQQKPGKAPKLLIYAASSL | (Human) | (Human) | (Human) | (Human) | | YLDY |
| | | | QSGVPSRFSGSGSGTDFTLTISSLQPED | | | | | | |
| | | | FATYYCQQSYSTPPEYTFGQGTKLEIK | | | | | | |
| | CC12.1 | 1293 | DIVMTQSPSFLSASVGDRVTITCRASQ | IGHV3-53 | IGHJ6 | IGKV1-9 | IGKJ3 | 2302 | ARDLDVYGLD |
| | | | GISSYLAWYQQKPGKAPKLLIYAASTL | (Human) | (Human) | (Human) | (Human) | | V |
| | | | QSGVPSRFSGSGSGTEFTLTISSLQPED | | | | | | |
| | | | FATYYCQQLNSYPPKFTFGPGTKVEIK | | | | | | |
| | CC12.10 | 1294 | QSALTQPASVSGSPGQSLTISCTGTSSD | IGHV1-2 | IGHJ4 | IGLV2-14 | IGU3 | 2303 | ARGPRYSGTH |
| | | | VGGYNYVSWYQQHPDKAPKLMIYDV | (Human) | (Human) | (Human) | (Human) | | FDY |
| | | | NNRPSGVSNRFSGSKSGSTASLTISGL | | | | | | |
| | | | QAEDEADYYCSSYAGSSTQVFGGGTK | | | | | | |
| | | | LTVL | | | | | | |
| | CC12.11 | 1295 | QSVLTQPASVSGSPGQSITISCTGTSSD | IGHV1-2 | IGHJ4 | IGLV2-14 | IGU3 | 2304 | ARGPRYSGTY |
| | | | VGGYNYVSWYQQHPGKVPKLMIYDV | (Human) | (Human) | (Human) | (Human) | | FDY |
| | | | SNRPSGVSNRFSGSKSGNTASLTISGL | | | | | | |
| | | | QAEDEADYYCSSYTSSSAQLFGGGTKL | | | | | | |
| | | | TVL | | | | | | |
| | CC12.12 | 1296 | QSALTQPASVSGSPGQSITISCTGTSSD | IGHV1-2 | IGHJ4 | IGLV2-14 | IGU3 | 2305 | ARGPRYSGTY |
| | | | VGGYNYVSWYQQHPDKAPKLMIYDV | (Human) | (Human) | (Human) | (Human) | | FDY |
| | | | SNRPSGVSNRFSGSKSGNTASLTISGL | | | | | | |
| | | | QAEDEADYYCSSYTSGSTQVFGGGTKL | | | | | | |
| | | | TVL | | | | | | |
| | CC12.13 | 1297 | DVVMTQSPSSLSASVGDRVTITCQAS | IGHV3-53 | IGHJ4 | IGKV1-33 | IGKJ5 | 2306 | ARDPYGYSSI |
| | | | QDISNYLNWYQQKPGKAPKLLIYDAS | (Human) | (Human) | (Human) | (Human) | | WDGQGGH |
| | | | NFATGVPSRFSGTGSGTDFTFTISSLQP | | | | | | |
| | | | EDIATYYCQQYDNLPITFGQGTRLEIK | | | | | | |
| | CC12.14 | 1298 | DVVMTQSPLSLPVTLGQPASISCRSSQ | IGHV3-21 | IGHJ6 | IGKV2-30 | IGKJ3 | 2307 | ARGGYCSDGS |
| | | | SLVYSDGNTYLNWFQQRPGQSPRRLI | (Human) | (Human) | (Human) | (Human) | | CYVQDRLIYYY |
| | | | YKVSNRDSGVPDRFSGSGSGTDFTLKI | | | | | | SGLDV |
| | | | SRVEAEDVGVFYCMQGTHWPPTFGQ | | | | | | |
| | | | GTKVDIK | | | | | | |
| | CC12.15 | 1299 | QAVLTQPPSVSGAPGQRVTISCTGSSS | IGHV3-48 | IGHJ4 | IGLV1-40 | IGU3 | 2308 | ARDRRRRYCT |
| | | | NIEAGYDVHWYQQLPGTAPKLLIYVN | (Human) | (Human) | (Human) | (Human) | | NGVCYRPEEI |
| | | | SNRPSGVPDRFSGSKSGTSASLAITGL | | | | | | DY |
| | | | QAEDEADYYCQSYDSSLSGVVFGGGT | | | | | | |
| | | | KLIVL | | | | | | |
| | CC12.16 | 1300 | SYELTQPPSVSVAPGQTARITCGGNNI | IGHV3-33 | IGHJ4 | IGLV3-21 | IGU3 | 2309 | ARDPFPGAV |
| | | | GSKSVHWYQQKPGQAPVLVVYDDSD | (Human) | (Human) | (Human) | (Human) | | AGTGYLQY |
| | | | RPSGIPERFSGSNSGNTATLTISRVEAG | | | | | | |
| | | | DEADYYCQVWDSSSDPWVFGGGTKL | | | | | | |
| | | | TVL | | | | | | |
| | CC12.17 | 1301 | SYELTQPPSVSVAPGKTARITCGGNNI | IGHV3-30 | IGHJ6 | IGLV3-21 | IGU3 | 2310 | AKSSGSYYYYY |
| | | | GSKSVHWYQQKPGQAPVLVVYDDTD | (Human) | (Human) | (Human) | (Human) | | YGMDV |
| | | | RPSGIPERFSGSSSGNTATLTISRVEAG | | | | | | |
| | | | DEADYYCQVWDSSSDHPVVFGGGTK | | | | | | |
| | | | LTVL | | | | | | |
| | CC12.18 | 1302 | NFMLTQPHSVSESPGKTVTISCTRSSG | IGHV1-46 | IGHJ4 | IGLV6-57 | IGU3 | 2311 | ARLHCGGDC |
| | | | SIANNYVKWYQQRPGSSPTTVIYEDN | (Human) | (Human) | (Human) | (Human) | | YLDY |
| | | | QRPSGVPDRFSGSIDSSSNSASLTISGL | | | | | | |
| | | | KTEDEADYYCQSYDSSNHEEIWVFGG | | | | | | |
| | | | GTKLIVL | | | | | | |
| | CC12.19 | 1303 | SYELTQSPSVSVAPGQTARITCGGNNI | IGHV3-23 | IGHJ6 | IGLV3-21 | IGU3 | 2312 | AKGSGSGSYP |
| | | | GSKSVHWYQRKPGQAPVLVVYDDSD | (Human) | (Human) | (Human) | (Human) | | NYYYYYGMD |
| | | | RPSGIPERFSGSNSGNTATLTISRVEAG | | | | | | V |
| | | | DEADYYCQVWDNNSDHLVFGGGTKL | | | | | | |
| | | | TVL | | | | | | |
| | CC12.2 | 1304 | EIVMTQSPGTLSLSPGERATLSCRASQ | IGHV3-53 | IGHJ4 | IGKV3-20 | IGKJ2 | 2313 | ARDYGDLYFD |
| | | | SVSSSYLAWYQQKPGQAPRLLIYGVSS | (Human) | (Human) | (Human) | (Human) | | Y |
| | | | RATGIPDRFSGSGSETDFTLTISRLEPE | | | | | | |
| | | | EFAVYYCQQYGSSPRTFGQGTKLEIK | | | | | | |
| | CC12.20 | 1305 | QSVLTQPPSASSTPGQRVTISCSGSSSN | IGHV3-30 | IGHJ6 | IGLV1-47 | IGU3 | 2314 | AKDQAYYDIL |
| | | | IGSNYVYWYQQLPGTAPKLLIYTNNQR | (Human) | (Human) | (Human) | (Human) | | TGYLNPPKNY |
| | | | PSGVPDRFSGSKSGTSASLAISGLRSED | | | | | | YYYGMDV |
| | | | EADYYCAAWDDSLSGRVVFGGGTKLT | | | | | | |
| | | | VL | | | | | | |
| | CC12.21 | 1306 | QAVVTQPPSASGTPGQRVTISCSGSSS | IGHV1-24 | IGHJ4 | IGLV1-44 | IGU3 | 2315 | ATAFSIFGVVP |
| | | | NIGSNTVNWYQQLPGTAPKLLIYSNN | (Human) | (Human) | (Human) | (Human) | | PDY |
| | | | QRPSGVPDRFSGSKSGTSASLAISGLQ | | | | | | |
| | | | SEDEADYYCAAWDDSLNGPVFGGGT | | | | | | |
| | | | KLTVL | | | | | | |
| | CC12.23 | 1307 | SYELTQPPSVSVSPGQTARITCSGDALP | IGHV4-39 | IGHJ4 | IGLV3-25 | IGU3 | 2316 | ARQGDCSTTS |
| | | | KQYAYWYQQKPGQAPVLVIYKDSERP | (Human) | (Human) | (Human) | (Human) | | CAYDY |
| | | | SGIPERFSGSSSGTTATLTISGVQAEDE | | | | | | |
| | | | ADYYCQSADSSGTYLVVFGGGTKLAV | | | | | | |
| | CC12.24 | 1308 | DIQLTQSPSSLSASVGDRVTITCRASQS | IGHV3-30 | IGHJ6 | IGKV1-39 | IGKJ1 | 2317 | AKDRTGNYYY |
| | | | ISSYLNWYQQKPGKAPKLLIYAASNLQ | (Human) | (Human) | (Human) | (Human) | | GMDV |
| | | | SGVPSRFSGSGSGTDFTLTISSLQPEDF | | | | | | |
| | | | ATYYCQQSYSTPWTFGQGTKVEIK | | | | | | |
| | CC12.25 | 1309 | QSALTQPPSASGTPGQRVTISCSGSSS | IGHV3-23 | IGHJ5 | IGLV1-44 | IGU3 | 2318 | AKDRYYEFWS |
| | | | NIGSNTVNWYQQLPGTAPKVLVYSND | (Human) | (Human) | (Human) | (Human) | | GYSNWFDP |
| | | | QRPSGVPDRFSGSKSGTSASLAISGLQ | | | | | | |
| | | | SEDEADYYCAAWDDSLNGPVFGGGT | | | | | | |
| | | | KLTVL | | | | | | |
| | CC12.26 | 1310 | QTVVTQEPSLTVSPGGTVTLTCVSSTG | IGHV5-51 | IGHJ4 | IGLV7-43 | IGU3 | 2319 | ARVNYYDSSG |
| | | | AVTSGYYPNWFQQKPGQAPRALIYST | (Human) | (Human) | (Human) | (Human) | | YPSFHFDY |
| | | | SKKHSWTPARFSGSLLGGKAALTLSGV | | | | | | |
| | | | QPEDEAEYYCLLYYGGAQRWVFGGG | | | | | | |
| | | | TKLTVL | | | | | | |
| | CC12.27 | 1311 | QSVLTQPASVSGSPGQSITISCTGTSSD | IGHV1-2 | IGHJ6 | IGLV3-23 | IGU1 | 2320 | AREMPAAM |
| | | | VGSYNLVSWYQQHPGKAPKLMIYEGS | (Human) | (Human) | (Human) | (Human) | | GYYYYGMDV |
| | | | KRPSGVSNRFSGSKSGNTASLTISGLQ | | | | | | |
| | | | AEDEADYYCYSYAGSSTFVFVFGTGTK | | | | | | |
| | | | VIVL | | | | | | |
| | CC12.28 | 1312 | QSALTQPPSASGTPGQRVTISCSGSSS | IGHV3-23 | IGHJ4 | IGLV1-47 | IGU3 | 2321 | AKANKYSSSE |
| | | | NIGSNYVYWYQQLPGAAPKLLIYRND | (Human) | (Human) | (Human) | (Human) | | FDF |
| | | | QRPSGVPDRFSGSKSGTSVSLAISGLRS | | | | | | |
| | | | EDEADYYCAAWDDSLSGWVFGGGTK | | | | | | |
| | | | LTVL | | | | | | |
| | CC12.3 | 1313 | EIVLTQSPGTLSLSPGERATLSCRASQS | IGHV3-53 | IGHJ4 | IGKV3-20 | IGKJ2 | 2322 | ARDFGDFYFD |
| | | | VSSYLAWYQQKPGQAPRLLIYGASSRA | (Human) | (Human) | (Human) | (Human) | | Y |
| | | | TGIPDRFSGSGSGTDFTLTISRLEPEDF | | | | | | |
| | | | AVYYCQQYGSSPRTFGQGTKLEIK | | | | | | |
| | CC12.4 | 1314 | QSVLTQPPSASGSPGQSVTISCTGTSS | IGHV1-2 | IGHJ3 | IGLV2-8 | IGU3 | 2323 | ATESWVYGS |
| | | | DVGGYDYVSWYQQHPGKAPKLMIYE | (Human) | (Human) | (Human) | (Human) | | GSYSSGAFDI |
| | | | VSKRPSGVPDRFSGSKSGNTASLTVPG | | | | | | |
| | | | LQAEDEADYYCTSYAGSNNFVFGGGT | | | | | | |
| | | | KLIVL | | | | | | |
| | CC12.5 | 1315 | QTVVTQPASVSGSPGQSITISCTGTSSD | IGHV1-2 | IGHJ4 | IGLV2-14 | IGU3 | 2324 | ARGPRYSGTY |
| | | | VGGYNYVSWYQQHPDKAPKLIIYDVS | (Human) | (Human) | (Human) | (Human) | | FDY |
| | | | NRPSGVSNRFSGSKSGNTASLTISGLQ | | | | | | |
| | | | AEDEAAYYCSSYTSSSTQVFGGGTKLT | | | | | | |
| | | | VL | | | | | | |
| | CC12.6 | 1316 | QSALTQPTSVSGSPGQSITISCTGTSSD | IGHV1-2 | IGHJ4 | IGLV2-14 | IGU3 | 2325 | ARGPRYSGTY |
| | | | VGGYNYVSWYQQHPGKAPKLIIYDVS | (Human) | (Human) | (Human) | (Human) | | FDY |
| | | | NRPSGVSNRFSGSKSGNTASLTISGLQ | | | | | | |
| | | | AEDEADYYCSSYTVSSTQVFGGGTKLT | | | | | | |
| | | | VL | | | | | | |
| | CC12.7 | 1317 | QPVLTQPASVSGSPGQSLTISCTGTSS | IGHV1-2 | IGHJ4 | IGLV2-14 | IGU3 | 2326 | ARGPRYSGTY |
| | | | DIGGFNYVSWYQQHPGKAPKLMIFD | (Human) | (Human) | (Human) | (Human) | | FDY |
| | | | VSKRPSGVPNRFSGSKSGNTASLTISGL | | | | | | |
| | | | QAEDEGDYYCSSYTISSAQVFGGGTKL | | | | | | |
| | | | TVL | | | | | | |
| | CC12.8 | 1318 | QSALTQPASVSGSPGQSITISCTGTSSD | IGHV1-2 | IGHJ4 | IGLV2-14 | IGU3 | 2327 | ARGPRYSGTY |
| | | | VGGYNHVSWYQQHPGKAPKLMIYDV | (Human) | (Human) | (Human) | (Human) | | FDY |
| | | | SNRPSGVSNRFSGSKSGNTASLTISGL | | | | | | |
| | | | QAEDEADYYCSSYTSSSAQLFGGGTKL | | | | | | |
| | | | TVL | | | | | | |
| | CC12.9 | 1319 | QSVLTQPASVSGSPGQSITISCTGTSSD | IGHV1-2 | IGHJ4 | IGLV2-14 | IGU3 | 2328 | ARGPRYSGTY |
| | | | VGGYNYVSWYQQHPGKVPKLMIYDV | (Human) | (Human) | (Human) | (Human) | | FDY |
| | | | SNRPSGVSNRFSGSKSGNTASLTISGL | | | | | | |
| | | | QAEDEADYYCSSYTSSSAQVFGGGTKL | | | | | | |
| | | | TVL | | | | | | |
| | CC6.29 | 1320 | DIQLTQSPSSLSASVGDRVTITCRASQT | IGHV7-4- | IGHJ5 | IGKV1-39 | IGKJ3 | 2329 | AVYYYDSGSP |
| | | | ASSYLNWYQQKPGKAPNLLIYAASSLQ | 1 | (Human) | (Human) | (Human) | | GWFDP |
| | | | SGVPSRFSGSGSVTDFTLTISSLQPEDF | (Human) | | | | | |
| | | | ATYYCQQSYSTPPTFGQGTKVDIK | | | | | | |
| | CC6.30 | 1321 | DIQMTQSPSSLSASVGDRVTITCRASQ | IGHV1-69 | IGHJ5 | IGKV1-39 | IGKJ3 | 2330 | ARDFRYCSST |
| | | | NISSYLNWYQQEAGKAPKLLIYAASSL | (Human) | (Human) | (Human) | (Human) | | RCYFWFDP |
| | | | QSGVPSRFSGSGSGTDFTLTISSLQPED | | | | | | |
| | | | FATYYCQQSYSTPRTFGQGTKVDIK | | | | | | |
| | CC6.31 | 1322 | DIQMTQSPSSLSASVGDRVTITCRASQ | IGHV1-46 | IGHJ4 | IGKV1-17 | IGKJ4 | 2331 | ARWYDSTGSI |
| | | | GIRNDLGWYQQKPGKAPKRLIYAASSL | (Human) | (Human) | (Human) | (Human) | | DY |
| | | | QSGVPSRFSGSGSGTEFTLTISSLQPED | | | | | | |
| | | | FATYYCLQHNSYPILTFGGGTKLEIK | | | | | | |
| | CC6.32 | 1323 | QLVLTQPPSVSVAPGKTARITCGGNNI | IGHV3-9 | IGHJ4 | IGLV3-21 | IGU6 | 2332 | AKDQGYSYG |
| | | | GSKSVYWYQQKPGQAPVLVVYDDSD | (Human) | (Human) | (Human) | (Human) | | NYFDY |
| | | | RPSGIPERFSGSNSGNTATLTISRVEAG | | | | | | |
| | | | DEADYYCQVWDSSSDHPYVFGSGTKV | | | | | | |
| | | | TVL | | | | | | |
| | CC6.33 | 1324 | EIVLTQSPGTLSLSPGERATLSCRASQS | IGHV1-69 | IGHJ4 | IGKV3-20 | IGKJ1 | 2333 | ALRNQWDLL |
| | | | VSSSYLAWYQQKPGQAPRLLIYGASSR | (Human) | (Human) | (Human) | (Human) | | VY |
| | | | ATGIPDRFSGSGSGTDFTLTISRLEPED | | | | | | |
| | | | FAVYYCQHYGSSLWTFGQGTKLEIK | | | | | | |
| | Clone11- | 1325 | DVLMTQTPLSLPVSLGDQASISCRSSQ | IGHV1-69 | IGHJ3 | IGKV1-117 | IGKJ5 | 2334 | ARRGYGSSYT |
| | 9 | | SIVHSNGNTYLEWYLQKPGQSPKLLIY | (Mouse) | (Mouse) | (Mouse) | (Mouse) | | WFAY |
| | | | KVSNRFSGVPDRFSGSGSGTDFTLKIS | | | | | | |
| | | | RVEAEDLGVYYCFQGSHVPLTFGAGT | | | | | | |
| | | | KLELK | | | | | | |
| | CnC2t1p1_ | 1326 | EIVLTQSPATLSLSPGERATLSCRASQS | IGHV1-69 | IGHJ4 | IGKV3-11 | IGKJ4 | 2335 | ARVSGYDSSG |
| | B10 | | VSSYLAWYQQKPGQAPRLLIYDASNR | (Human) | (Human) | (Human) | (Human) | | YWGDY |
| | | | ATGIPARFSGSGSGTDFTLTISSLEPED | | | | | | |
| | | | FAVYYCQQRSNWPPALTFGGGTKVEIK | | | | | | |
| | CnC2t1p1_ | 1327 | QSALTQPASVSGSPGQSITISCTGTSSD | IGHV1-18 | IGHJ5 | IGLV2-23 | IGU3 | 2336 | ARDGELLGW |
| | B4 | | VGSYNLVSWYQQHPGKAPKLMIYEGS | (Human) | (Human) | (Human) | (Human) | | FDP |
| | | | KRPSGVSNRFSGSKSGNTASLTISGLQ | | | | | | |
| | | | AEDEADYYCCSYAGSSTWVFGGGTKL | | | | | | |
| | | | TVL | | | | | | |
| | CnC2t1p1_ | 1328 | DIVMTQSPLSLPVTPGEPASISCRSSQS | IGHV3-49 | IGHJ6 | IGKV2-28 | IGKJ3 | 2337 | TRVRRLWFGS |
| | D6 | | LLHSNGYNYLDWYLQKPGQSPQLLIYL | (Human) | (Human) | (Human) | (Human) | | YYYGMDV |
| | | | GSNRASGVPDRFSGSGSGTDFTLKISR | | | | | | |
| | | | VEAEDVGVYYCMQALQTPGTFGPGT | | | | | | |
| | | | KVDIK | | | | | | |
| | CnC2t1p1_ | 1329 | DIVMTQSPLSLPVTPGEPASISCRSSQS | IGHV3-49 | IGHJ6 | IGKV2-28 | IGKJ3 | 2338 | TRVRRLWFGS |
| | E12 | | LLHSNGYNYLDWYLQKPGQSPQLLIYL | (Human) | (Human) | (Human) | (Human) | | YYYGMDV |
| | | | GSNRASGVPDRFSGSGSGTDFTLKISR | | | | | | |
| | | | VEAEDVGVYYCMQALQTPGTFGPGT | | | | | | |
| | | | KVDIK | | | | | | |
| | CnC2t1p1_ | 1330 | QSALTQPASVSGSPGQSITISCTGTSGD | IGHV1-2 | IGHJ4 | IGLV2-23 | IGU3 | 2339 | ARASVSTITDF |
| | E8 | | VGSYNLVSWYQQHPGKAPKLVIYEAT | (Human) | (Human) | (Human) | (Human) | | DY |
| | | | KRPSGVSNRFFASKSGNTASLTISGLQ | | | | | | |
| | | | AEDEADYYCCSYAGVRTVVFGGGTKL | | | | | | |
| | | | TVL | | | | | | |
| | CnC2t1p1_ | 1331 | QSALTQPASVSGSPGQSITISCTGTSGD | IGHV1-2 | IGHJ4 | IGLV2-23 | IGU3 | 2340 | ARASVATITDF |
| | G6 | | IGSYNLVSWYQQYPGKAPKLIIYEASKR | (Human) | (Human) | (Human) | (Human) | | DY |
| | | | PSGVSNRFFASKSGNTASLTISGLQAE | | | | | | |
| | | | DEADYYCCSYAGVRTVVFGGGTKLTVL | | | | | | |
| | COV2- | 1332 | EIVLTQSPGTLSLSPGERATLSCRASQS | IGHV3- | IGHJ4 | IGKV3-20 | IGKJ1 | 2341 | ARPQSGGYYA |
| | 2006 | | VSSSYLAWYQQKPGQAPRLLIYGASSR | 303 | (Human) | (Human) | (Human) | | PLDY |
| | | | ATGIPDRFSGSGSGTDFTLTISRLEPED | (Human) | | | | | |
| | | | FAVYYCQQYGSSPWTFGQGTKVEIK | | | | | | |
| | COV2- | 1333 | DIQMTQSPSSLSASVGDRVTITCQASQ | IGHV3-30 | IGHJ6 | IGKV1-33 | IGKJ3 | 2342 | AKVSATYYYYY |
| | 2007 | | DISTYLNWYQQKPGKAPKLLIYDASNL | (Human) | (Human) | (Human) | (Human) | | YGMDV |
| | | | ETGVPSRFSGSGSGTDFTFTISSLQPED | | | | | | |
| | | | IATYYCQQYDNLLFTFGPGTKVDVK | | | | | | |
| | COV2- | 1334 | QSALTQPASVSGSPGQSITISCTGTSGD | IGHV3-30 | IGHJ4 | IGLV2-14 | IGU3 | 2343 | ARDTATYVLL |
| | 2009 | | VGAYNYVSWYQQHPVKAPKLMIYDV | (Human) | (Human) | (Human) | (Human) | | WSGDFNLDY |
| | | | SKRPSGVSNRFSGSKSGNTASLTISGLQ | | | | | | |
| | | | TEDEADYYCSSYTSSSTLWVFGGGTKL | | | | | | |
| | | | TVL | | | | | | |
| | COV2- | 1335 | ITCRASQSISSYLNWYQQKPGKAPKLLI | IGHV3- | IGHJ6 | IGKV1-39 | IGKJ3 | 2344 | ARGHTGNYYY |
| | 2011 | | YAASSLQSGVPSRFGGSASGTDFTLTIS | 303 | (Human) | (Human) | (Human) | | GMDV |
| | | | SLQPEDFATYYCQQSYSTFTFGPGTKV | (Human) | | | | | |
| | | | DIK | | | | | | |
| | COV2- | 1336 | DIVMTQSPDFLAVSLGERATINCKSSQ | IGHV3-30 | IGHJ4 | IGKV4-1 | IGKJ4 | 2345 | AKGGDSSGW |
| | 2013 | | SVLHTPKNKNYLAWYKQKPGQPPKVL | (Human) | (Human) | (Human) | (Human) | | AWDGDNPPT |
| | | | IYWASTRESGVPERFSGSGSGTDFTLII | | | | | | DY |
| | | | SSLQAEDAAVYYCQQYYTAPLTFGGG1 | | | | | | |
| | | | KVEIK | | | | | | |
| | COV2- | 1337 | EIVLTQSPATLSLSPGERATLSCRASQS | IGHV3-9 | IGHJ4 | IGKV3-11 | IGKJ2 | 2346 | AMGPFGELLP |
| | 2015 | | VSSYLAWYQQKPGQAPRLLIYDTSNR | (Human) | (Human) | (Human) | (Human) | | YYFDY |
| | | | ATGIPARFSGSGSGTDFTLTISSLEPED | | | | | | |
| | | | FAVYYCQQRSNWPPYTFGQGTKLEIK | | | | | | |
| | COV2- | 1338 | QSALTQPASVSGSPGQSITISCTGTSSD | IGHV1-24 | IGHJ5 | IGLV2-14 | IGU3 | 2347 | AAAPAVMTA |
| | 2016 | | VGGYHYVSWYQHHPGKAPKLIIYDVIK | (Human) | (Human) | (Human) | (Human) | | GWFDP |
| | | | RPSGVSNRFSGSKSGNTASLTISGLQA | | | | | | |
| | | | EDEAYYYCSSYTSSTTWVFGGGTRLTV | | | | | | |
| | | | L | | | | | | |
| | COV2- | 1339 | QSVLTQPPSVSGAPGQRVTISCTGSSS | IGHV4-59 | IGHJ5 | IGLV1-40 | IGU1 | 2348 | VRGAMAWF |
| | 2017 | | NIGAGYNVHWYQQLPGTAPKLLIYGN | (Human) | (Human) | (Human) | (Human) | | DP |
| | | | NNRPSGVPDRFSGSKSGTSASLAITGL | | | | | | |
| | | | QAEDEADYYCQSFDSSLSGSDVFGTGT | | | | | | |
| | | | KVSVL | | | | | | |
| | COV2- | 1340 | QSVLTQPPSASGTPGQRVTISCSGSSS | IGHV1-24 | IGHJ4 | IGLV1-47 | IGU2 | 2349 | ATQPAAIGGT |
| | 2021 | | NIGSNYVYWYQQLPGTAPKLLIYRNN | (Human) | (Human) | (Human) | (Human) | | PPYY |
| | | | QRPSGVPDRFSGSKSGTSASLAISGLRS | | | | | | |
| | | | EDEADYYCAAWDASLSGHVVFGGGT | | | | | | |
| | | | KLTVL | | | | | | |
| | COV2- | 1341 | DIVMAQTPLSLPVTPGEPASISCRSSQS | IGHV1-18 | IGHJ6 | IGKV2-40 | IGKJ2 | 2350 | ARDQGPTYYY |
| | 2022 | | LLDSDDGNTYFDWYLQKPGQSPQLLI | (Human) | (Human) | (Human) | (Human) | | GSGSPHYGM |
| | | | YTLSYRASGVPDRFSGSGSGTDFTLKIN | | | | | | DV |
| | | | RVEAEDVGVYYCMQRIEFPWTFGQG | | | | | | |
| | | | TKLEIK | | | | | | |
| | COV2- | 1342 | DIVMTQSPDFLAVSLGERATINCKSSQ | IGHV3-30 | IGHJ4 | IGKV4-1 | IGKJ4 | 2351 | AKGGDSSGW |
| | 2025 | | SVLHTPKNKNYLAWYKQKPGQPPKVL | (Human) | (Human) | (Human) | (Human) | | AWDGDNPPT |
| | | | IYWASTRESGVPERFSGSGSGTDFTLII | | | | | | DY |
| | | | SSLQAEDAAVYYCQQYYTAPLTFGGG1 | | | | | | |
| | | | KVEIK | | | | | | |
| | COV2- | 1343 | DIQMTQSPSSLSASVGDRVTITCRASQ | IGHV1-24 | IGHJ6 | IGKV1-16 | IGKJ1 | 2352 | ATSFPIRGDPS |
| | 2026 | | GITNYLAWFQQKPGKAPKSLIYAVSSL | (Human) | (Human) | (Human) | (Human) | | YYYYYYGMDV |
| | | | QSGVPSKFSGSGSGTDFTLTISSLQPED | | | | | | |
| | | | FATYYCQQYNSYPWTFGQGTKVEIK | | | | | | |
| | COV2- | 1344 | SYELTQPPSVSVSPGQTARITCSGDALP | IGHV3-30 | IGHJ6 | IGLV3-25 | IGU3 | 2353 | AIYGYYYYGLD |
| | 2027 | | KQYAYWYQQKPGQAPVLVIYKDSERP | (Human) | (Human) | (Human) | (Human) | | V |
| | | | SGIPERFSGSSSGTTVTLTISGVQAEDE | | | | | | |
| | | | ADYYCQSADSSGTYFWVFGGGTKLTV | | | | | | |
| | | | L | | | | | | |
| | COV2- | 1345 | DIVMTQSPDFLAVSLGERATINCKSSQ | IGHV3-30 | IGHJ4 | IGKV4-1 | IGKJ4 | 2354 | AKGGDSSGW |
| | 2028 | | SVLHTPKNKNYLAWYKQKPGQPPKVL | (Human) | (Human) | (Human) | (Human) | | AWDGDNPPT |
| | | | IYWASTRESGVPERFSGSGSGTDFTLII | | | | | | DY |
| | | | SSLQAEDAAVYYCQQYYTAPLTFGGG1 | | | | | | |
| | | | KVEIK | | | | | | |
| | COV2- | 1346 | QSVLTQPPSVSGTPGQRVTISCSGSNS | IGHV4-4 | IGHJ3 | IGLV1-44 | IGU3 | 2355 | ARPTAGAGG |
| | 2029 | | KIGSYSVNWYQQLPGTAPKLLIYRNNQ | (Human) | (Human) | (Human) | (Human) | | AFDI |
| | | | RPSGVPDRFFGSKSGTSASLAISGLQSE | | | | | | |
| | | | DEADYYCSVWDDSLNGPLFGGGTKLT | | | | | | |
| | | | FV | | | | | | |
| | COV2- | 1347 | SYELTQPPSVSVSPGQTARITCSGDAW | IGHV2-26 | IGHJ5 | IGLV3-25 | IGU3 | 2356 | ARTEWLLSDN |
| | 2031 | | PNQYAYWYQQKPGQAPVLVIYKDTER | (Human) | (Human) | (Human) | (Human) | | WFDS |
| | | | PSGIPERFSGSSSGTTVTLTISGVQAED | | | | | | |
| | | | EADYFCQSSDSSGVVFGGGTKLTVL | | | | | | |
| | COV2- | 1348 | DIQMTQSPSTLSASVGDRVTITCRASQ | IGHV1-46 | IGHJ3 | IGKV1-5 | IGKJ1 | 2357 | ARGGLVPAA |
| | 2032 | | SISSWLAWYQQKPGKAPKLLIYKASSL | (Human) | (Human) | (Human) | (Human) | | RNAFDI |
| | | | ESGVPSRFSGSGSGTEFTLTISSLQPDD | | | | | | |
| | | | FATYYCQQYNSYSWTFGQGTKVEIK | | | | | | |
| | COV2- | 1349 | QSVLTQPPSASGTPGQRVTISCSGSNS | IGHV4-4 | IGHJ3 | IGLV1-44 | IGU3 | 2358 | ARPTAGAGG |
| | 2033 | | KIGSYSVNWYQQLPGTAPKLLIYRNNQ | (Human) | (Human) | (Human) | (Human) | | AFDT |
| | | | RPSGVPDRFSGSKSGTSASLAISGLQSE | | | | | | |
| | | | DEADYYCSVWDDSLNGPLFGGGTKLT | | | | | | |
| | | | FL | | | | | | |
| | COV2- | 1350 | DIQMTQSPSSLSASVGDRVTITCRASQ | IGHV1-18 | IGHJ5 | IGKV1-39 | IGKJ4 | 2359 | ARDLPIKVVV |
| | 2034 | | SISSYLNWYQQKPGKAPKLLIYAASSLQ | (Human) | (Human) | (Human) | (Human) | | PAADYNWFD |
| | | | SGVPSRFSGSGSGTDFTLTISSLQPEDF | | | | | | P |
| | | | ATYYCQQSYSTPPTFGGGTKVQIK | | | | | | |
| | COV2- | 1351 | DIQMTQSPSSLSASVGDRVTITCRASQ | IGHV2-5 | IGHJ3 | IGKV1-39 | IGKJ1 | 2360 | AHRRGILTED |
| | 2035 | | SISSYLNWYQQRPGKAPKLLIYAASSLQ | (Human) | (Human) | (Human) | (Human) | | AFDI |
| | | | SGVPSRFSGSGYGTDFTLTISSLQPEDF | | | | | | |
| | | | ATYYCQQSYNTPRTFGQGTKVEIK | | | | | | |
| | COV2- | 1352 | DIQMTQSPSSVSASVGDRVTITCRASQ | IGHV3-53 | IGHJ6 | IGKV1-12 | IGKJ5 | 2361 | ARDLNEHGL |
| | 2037 | | GISSWLAWYQQKPGKAPKLLIYAASSL | (Human) | (Human) | (Human) | (Human) | | DV |
| | | | QSGVPSRFSGSGSGTDFTLTISSLQPED | | | | | | |
| | | | FATYYCQQTNSFPTFGQGTRLEIR | | | | | | |
| | COV2- | 1353 | EIVLTQSPGTLSLSPGERATLSCRASQS | IGHV1-46 | IGHJ3 | IGKV3-20 | IGKJ3 | 2362 | ARGTLIPAHR |
| | 2039 | | VSSSFLAWYQQKPGQAPRLLIYGASN | (Human) | (Human) | (Human) | (Human) | | GAFDI |
| | | | RATGIPDRFSGSGSGTDFTLTISRLEPE | | | | | | |
| | | | DFAVYYCQQYGNSPQFGPGTKVDIK | | | | | | |
| | COV2- | 1354 | DIQMTQSPSSLSASVGDRVTITCRASQ | IGHV4-61 | IGHJ6 | IGKV1-5 | IGKJ1 | 2363 | ARARPDYYYY |
| | 2041 | | SISNWLAWYQQKPGKAPKLLIYKASSL | (Human) | (Human) | (Human) | (Human) | | YAMDV |
| | | | ESGVPSRFSGSGSGTEFTLTISSLQPDD | | | | | | |
| | | | FATYYCQQYNSYSTWTFGQGTKVEIK | | | | | | |
| | COV2- | 1355 | DIQMTQSPSSLSASVGDRVTITCRASQ | IGHV3-9 | IGHJ6 | IGKV1-39 | IGKJ2 | 2364 | AKAHSTGHQ |
| | 2046 | | SISSFLNWYQQKPGKAPKLLIYAAFNL | (Human) | (Human) | (Human) | (Human) | | YYYGMDV |
| | | | QSGVPSRFSGSGSGTDFTLTISSLQSED | | | | | | |
| | | | FATYYCQQSYNTPYTFGQGTKLEIK | | | | | | |
| | COV2- | 1356 | QSVLTQPPSASGTPGQRVIISCSGSSSN | IGHV1-2 | IGHJ4 | IGLV1-44 | IGU2 | 2365 | ARVVVLGYGR |
| | 2050 | | IGSNTVKWYHQLPGTAPKLLICSNNQR | (Human) | (Human) | (Human) | (Human) | | PNNYYDGRN |
| | | | PSGVPDRFSGSKSDTSASLAISGLQSED | | | | | | VWDY |
| | | | EADYYCAAWDDSLNALVFGGGTKLTV | | | | | | |
| | | | L | | | | | | |
| | COV2- | 1357 | DIQMTQSPSSLSASVGDRVTITCRASQ | IGHV2-70 | IGHJ4 | IGKV1-39 | IGKJ5 | 2366 | ARGVVTYDY |
| | 2051 | | SIAGYLNWYQQKPGKAPKLLIYGTTSL | (Human) | (Human) | (Human) | (Human) | | |
| | | | QSGVPVRFSGSGSGTDFTLTISSLQPE | | | | | | |
| | | | DFATYYCQQSYSTPGTFGQGTRLEIK | | | | | | |
| | COV2- | 1358 | DIQMTQSPSSLSASVGDRVTITCQASQ | IGHV1-69 | IGHJ6 | IGKV1-33 | IGKJ2 | 2367 | ARGRGYSNY |
| | 2054 | | DINHYLNWYQQKPGKAPKLLIYDASN | (Human) | (Human) | (Human) | (Human) | | GASYYMDV |
| | | | LETGVPSRFSGSGSGTDFTFTITSLQPE | | | | | | |
| | | | DVATYYCQQSDNLPMYTFGQGTKLEI | | | | | | |
| | | | K | | | | | | |
| | COV2- | 1359 | SYVLTQPPSVSVAPGKTARITCEGNNI | IGHV3-9 | IGHJ6 | IGLV3-21 | IGU2 | 2368 | AKGRGAGYTS |
| | 2055 | | GSKSVHWYQQKPGQAPVLVVYDDSG | (Human) | (Human) | (Human) | (Human) | | YMDV |
| | | | RPSGIPERFSGSNSGNTATLTISRVEAG | | | | | | |
| | | | DEADYFCQVWDSSSDHHVVFGGGTK | | | | | | |
| | | | LTVL | | | | | | |
| | COV2- | 1360 | QSVLTQPPSASGTPGQRVTISCSGSNS | IGHV1-8 | IGHJ4 | IGLV1-44 | IGU2 | 2369 | ARMRTGWPT |
| | 2064 | | NIGSYTVNWYQQFPGTAPKLLIYDNN | (Human) | (Human) | (Human) | (Human) | | HGRPDDF |
| | | | QRTSGVPDRFSGSKSGTSASLAISGLQ | | | | | | |
| | | | SEDEANYYCLVWDDSLNGLVFGGGTK | | | | | | |
| | | | LTVL | | | | | | |
| | COV2- | 1361 | QSVLTQPPSVSGAPGQRVTISCTGSSS | IGHV3-53 | IGHJ3 | IGLV1-40 | IGU2 | 2370 | ARSYDILTGYR |
| | 2068 | | NIGSGSDVHWYQQLPGTAPKLLIYGN | (Human) | (Human) | (Human) | (Human) | | DAFDI |
| | | | TNRPSGVPDRFSGSKSGTSASLAITGL | | | | | | |
| | | | QAEDEADYYCQSYDSRLSGFVVFGGG | | | | | | |
| | | | TKLTVL | | | | | | |
| | COV2- | 1362 | DIQMTQSPSSLSASVGDRVTITCRASQ | IGHV4-34 | IGHJ6 | IGKV1-39 | IGKJ4 | 2371 | ARVGYSQGYY |
| | 2070 | | SISNYLNWYQQKPGKAPKLLIYAASSL | (Human) | (Human) | (Human) | (Human) | | YYYMDV |
| | | | QSGVPSRFSGSGSGTDFSLTISSLQPED | | | | | | |
| | | | FATYSCQQSYTTLLTFGGGTKVEIK | | | | | | |
| | COV2- | 1363 | EIVLTQSPGTLSLSPGERATLSCRASQS | IGHV1-58 | IGHJ3 | IGKV3-20 | IGKJ1 | 2372 | AAPHCSRTSC |
| | 2072 | | VSSSYLGWYQQKPGQAPRLLIYGASSR | (Human) | (Human) | (Human) | (Human) | | YDAFDL |
| | | | ATGIPDRFSGSGSGTDFTLTISRLEPED | | | | | | |
| | | | FAVYYCQQYGSSPWTFGQGTKVEIK | | | | | | |
| | COV2- | 1364 | QSVLTQPPSVSGAPGQRVTLSCTGSSS | IGHV1-69 | IGHJ2 | IGLV1-40 | IGU3 | 2373 | ARVGVSGFKS |
| | 2078 | | SIGAGYDVHWYQQLPGTAPKLLIYGS | (Human) | (Human) | (Human) | (Human) | | GSSWYFDL |
| | | | SSRPSGVPDRFSGSKSGTSASLAITGL | | | | | | |
| | | | QAEDEADYYCQSYDSSLSDSVFGGGT | | | | | | |
| | | | KVTVL | | | | | | |
| | COV2- | 1365 | DIQLTQSPSFLSASVGDRVTITCRASQG | IGHV3-66 | IGHJ6 | IGKV1-9 | IGKJ5 | 2374 | ARDLVTYGLD |
| | 2080 | | ISSYLAWYQQKPGTAPSLLIYAASTLQ | (Human) | (Human) | (Human) | (Human) | | V |
| | | | SGVPSRFSGSGSGTEFTLTISSLQPEDF | | | | | | |
| | | | ATYYCQLLSSHPLTFGQGTRLEIK | | | | | | |
| | COV2- | 1366 | SSELTQDPAVSVALGQTVRITCQGDSL | IGHV3-20 | IGHJ3 | IGLV3-19 | IGU3 | 2375 | AVIMSPIPRYS |
| | 2082 | | RSYYASWYQQKPGQVPILVIYDKSSR | (Human) | (Human) | (Human) | (Human) | | GYDWAGGAF |
| | | | PSGIPDRFSGSSSGSTASLTITGAQAED | | | | | | DI |
| | | | EADYYCSSRDSSGSAVVFGGGTKLTV | | | | | | |
| | | | L | | | | | | |
| | COV2- | 1367 | DIQMTQSPSSLSASVGDRVTITCQASQ | IGHV3-30 | IGHJ4 | IGKV1-33 | IGKJ3 | 2376 | AKSLGPYCSG |
| | 2083 | | DISSYLSWYQQKPGKAPKLLIYDASSL | (Human) | (Human) | (Human) | (Human) | | GTCYSLVGDY |
| | | | ETGVPSRFSGSGSGTDFTFTISSLQPED | | | | | | |
| | | | IATYYCQQYASLPFTFGPGTKVDIK | | | | | | |
| | COV2- | 1368 | SSELTQDPAVSVALGQTVRITCQGDSL | IGHV3-20 | IGHJ3 | IGLV3-19 | IGU2 | 2377 | AVIMSPIPRYS |
| | 2094 | | RSYYASWYQQKPGQVPILVIYDKSSR | (Human) | (Human) | (Human) | (Human) | | GYDWAGDAF |
| | | | PSGIPDRFSGSSSGSTASLTITGAQAED | | | | | | DI |
| | | | EADYYCSSRDSSGSAVVFGGGTKLTV | | | | | | |
| | | | L | | | | | | |
| | COV2- | 1369 | QSVLTQAPSASGTPGQRVTISCSGSSS | IGHV1-8 | IGHJ4 | IGLV1-44 | IGU2 | 2378 | ARMRSGWPT |
| | 2096 | | SIGSYTISWYQQLPGTAPKLLIYGSDQ | (Human) | (Human) | (Human) | (Human) | | HGRPDDF |
| | | | RTSGVPDRFSGSKFGTSASLAISGLQSE | | | | | | |
| | | | DESSYYCAVWDDSLSGLVFGGGTKL | | | | | | |
| | | | TVL | | | | | | |
| | COV2- | 1370 | DIQMTQSPSSLSASVGDRVTITCRASQ | IGHV3-9 | IGHJ6 | IGKV1-39 | IGKJ1 | 2379 | AKDIIRQGED |
| | 2097 | | SIASYLSWYQQKPGKAPKLLIYAASSL | (Human) | (Human) | (Human) | (Human) | | GMDV |
| | | | QSGVPSRFSGSGSGTDFTLTISSLQPEE | | | | | | |
| | | | FATYYCQQSYSTPWTFGQGTKVEIK | | | | | | |
| | COV2- | 1371 | DIVMTQSPATLSVSPGERAILSCRASQ | IGHV3-23 | IGHJ3 | IGKV3-15 | IGKJ1 | 2380 | VKGLFDWFPL |
| | 2098 | | SVRSSLAWYQQKPGQAPRLLISGAST | (Human) | (Human) | (Human) | (Human) | | |
| | | | RATAIPARFSGSGSGTEFTLTITSLQSE | | | | | | |
| | | | DCAVYYCHQYSSWPQTFGQGTKVEIK | | | | | | |
| | COV2- | 1372 | SFMLTQPHSVSESPGKTVTISCTGSSG | IGHV3-7 | IGHJ4 | IGLV6-57 | IGU3 | 2381 | ARLGFYYGGA |
| | 2103 | | SIASSYVQWYQQRPGSAPTTVISEDS | (Human) | (Human) | (Human) | (Human) | | DY |
| | | | QRPSGVPDRFSGSIDSSSSSASLTISGL | | | | | | |
| | | | KTEDEADYYCQSYDGISRAWVFGGGT | | | | | | |
| | | | KLTVL | | | | | | |
| | COV2- | 1373 | DIVMTQSPLSLPVTPGEPASISCRSSQS | IGHV3-9 | IGHJ4 | IGKV2-28 | IGKJ5 | 2382 | AKGVDYGGK |
| | 2108 | | LLHSLGYSSLSWYLQKPGQSPHLLIYL | (Human) | (Human) | (Human) | (Human) | | LAYFDS |
| | | | GSSRASGVPDRFSGSGSATDFTLKISR | | | | | | |
| | | | LEAEDVGVYYCMQALQTPLTFGQGTR | | | | | | |
| | | | LEIK | | | | | | |
| | COV2- | 1374 | DIQMTQSPSSLSACVGDRVTITCRASQ | IGHV3- | IGHJ4 | IGKV1-39 | IGKJ5 | 2383 | ARDIDSGYDP |
| | 2110 | | SISSYLSWYQQKPGKGPKLLIYAASSLQ | 303 | (Human) | (Human) | (Human) | | TPVFDY |
| | | | SGVPSRFSGSGSGTDFTLTISSLQPEDF | (Human) | | | | | |
| | | | ATYYCQQSYSSLSITFGQGTRLEIK | | | | | | |
| | COV2- | 1375 | DIQMTQSPSSLSASVGDRVTITCRASQ | IGHV3-13 | IGHJ5 | IGKV1-39 | IGKJ1 | 2384 | ARVLYDSSGF |
| | 2111 | | SISSYLSWYQQKPGKAPKLLIYAASSLE | (Human) | (Human) | (Human) | (Human) | | YSWFDP |
| | | | SGVPSRFSGSGSGTDFTLTISSLQPEDF | | | | | | |
| | | | ATYYCQQSYEIPPWTFGQGTKVEIK | | | | | | |
| | COV2- | 1376 | DIQMTQSPSSLSASVGDRVTITCQASQ | IGHV3-53 | IGHJ5 | IGKV1-33 | IGKJ4 | 2385 | ARDFLRWHD |
| | 2113 | | DISSYLSWYQQKPGKAPKLLIYDASS | (Human) | (Human) | (Human) | (Human) | | L |
| | | | LETGVPLRFSGSGSGTDFIFTISSLQPE | | | | | | |
| | | | DIATYYCQQYDSLPPVFGGGTKVEIK | | | | | | |
| | COV2- | 1377 | QSVLTQPPSVSGAPGQRVTISCTGSSS | IGHV1-69 | IGHJ6 | IGLV1-40 | IGU2 | 2386 | ARGRGYSNY |
| | 2114 | | NIGAGYDVHWYQQLPETAPKLLIYAN | (Human) | (Human) | (Human) | (Human) | | GASYYMDV |
| | | | SNRPSGVPDRFSGSKSGTSASLAITGL | | | | | | |
| | | | QAEDEADYYCQSYDSSLSGSVFGGGT | | | | | | |
| | | | KLTVL | | | | | | |
| | COV2- | 1378 | NFMLTQPHSVSESPGKTVTISCTGSSG | IGHV4-39 | IGHJ5 | IGLV6-57 | IGU2 | 2387 | ARILVIFTLNW |
| | 2128 | | SIASNYVQWYQQRPGSAPTTVIYEDN | (Human) | (Human) | (Human) | (Human) | | FDP |
| | | | QRPSGVPDRFSGSIDSSSNSASLTISGL | | | | | | |
| | | | KTEDEADYYCQSYDSGNPIFGGGTKLT | | | | | | |
| | | | VL | | | | | | |
| | COV2- | 1379 | DIVMTQSPDSLAVSLGERATINCKSSQ | IGHV3-15 | IGHJ4 | IGKV4-1 | IGKJ4 | 2388 | TTAGSYYYDT |
| | 2130 | | SVLYSSNNKNYLAWYQQKPGQPPKLL | (Human) | (Human) | (Human) | (Human) | | VGPGLPEGKF |
| | | | MYWASTRESGVPDRFSGSGSGAEFTL | | | | | | DY |
| | | | TISSLQAEDVAIYYCQQYYSTLTFGGGT | | | | | | |
| | | | KVEIK | | | | | | |
| | COV2- | 1380 | DIQMTQSPSSLSASVGDRVTITCQASQ | IGHV3-53 | IGHJ5 | IGKV1-33 | IGKJ4 | 2389 | ARDFLRWHD |
| | 2132 | | DINNYLNWYQQKPGKAPKLLIYDASN | (Human) | (Human) | (Human) | (Human) | | L |
| | | | LETGVPLRFSGSGSGTDFTFTISSLQPE | | | | | | |
| | | | DIATYYCQQYDNLPPVFGGGTKVEIK | | | | | | |
| | COV2- | 1381 | NFMLTQPHSVSESPGKTVTFSCTGSSG | IGHV4-61 | IGHJ4 | IGLV6-57 | IGU2 | 2390 | AGSPVPPTIV |
| | 2137 | | SIASNYVQWYQQRPGSAPTTVIYEDN | (Human) | (Human) | (Human) | (Human) | | GASY |
| | | | QRPSGVPDRFSGSIDSSSNSASLTISGL | | | | | | |
| | | | KTEDEADYYCQSYDGINRWLVFGGGT | | | | | | |
| | | | KLTVL | | | | | | |
| | COV2- | 1382 | QPVLTQPPSASASLGASVTLTCTLSSGY | IGHV5-51 | IGHJ2 | IGLV9-49 | IGU2 | 2391 | ARRGEAAGI |
| | 2142 | | SNYKVDWYQQRPGKGPRFVMRVGT | (Human) | (Human) | (Human) | (Human) | | WYFDL |
| | | | GGIVGSKGDGIPDRFSVLGSGLNRYLTI | | | | | | |
| | | | KNIQEEDESDYHCGADHGSGSNFEYV | | | | | | |
| | | | VFGGGTKLTVL | | | | | | |
| | COV2- | 1383 | QSVVTQPPSASGTPGQRVTISCSGSSS | IGHV3-66 | IGHJ6 | IGLV1-44 | IGU1 | 2392 | AKEGGSGSLR |
| | 2143 | | NIGYNIVNWYQQLPGTAPKLLIYSNN | (Human) | (Human) | (Human) | (Human) | | YYYYGMDV |
| | | | QRPSGVPDRFSGSKSGTSASLSISGLQS | | | | | | |
| | | | EDEADYYCAAWDDSLNGYVFGTGTK | | | | | | |
| | | | VTVL | | | | | | |
| | COV2- | 1384 | DIQLTQSPSFLSASVGDRVTITCRASQG | IGHV3-48 | IGHJ6 | IGKV1-9 | IGKJ1 | 2393 | ARRSYRSSWY |
| | 2146 | | ISSYLAWYQQKPGKAPKLLIYAASTLQS | (Human) | (Human) | (Human) | (Human) | | YYYGMDV |
| | | | GVPSRFSGSGSGTEFTLTISSLQPEDFA | | | | | | |
| | | | TYYCQQLNSYPVTFGQGTKVEIK | | | | | | |
| | COV2- | 1385 | QSALTQPASVSGSPGQSITISCTGTSSD | IGHV3- | IGHJ6 | IGLV2-14 | IGU1 | 2394 | ARSTSGSYYY |
| | 2147 | | VGDYNYVSWYQQHPGKAPKLMIYDV | 303 | (Human) | (Human) | (Human) | | GMDV |
| | | | SNRPSGVSNRFSGSKSGNTASLTISGL | (Human) | | | | | |
| | | | QAEDEAEYYCSSYTSSSTLLYVFGTGTK | | | | | | |
| | | | VTVL | | | | | | |
| | COV2- | 1386 | SYVLTQPPSVSVAPGKTARITCGGNNI | IGHV3- | IGHJ3 | IGLV3-21 | IGU3 | 2395 | ARDWAPTYY |
| | 2150 | | GNKGVHWYQQKPGQAPVLVVDDDS | 303 | (Human) | (Human) | (Human) | | DMPSAFDI |
| | | | DRPSGIPERFSGSNSGNTATLIISSVEV | (Human) | | | | | |
| | | | GDEADFYCQVWDSSSDHPGVFGGGT | | | | | | |
| | | | KLTVL | | | | | | |
| | COV2- | 1387 | EIVLTQSPATLSLSPGERATLSCRASQS | IGHV1-69 | IGHJ1 | IGKV3-11 | IGKJ4 | 2396 | ARIGSYPEYFQ |
| | 2151 | | VSSFLAWYQQKPGQAPRLLIYDASNR | (Human) | (Human) | (Human) | (Human) | | |
| | | | ATGIPARFSGSGSGTDFTLTISSLEPED | | | | | | |
| | | | FAVYYCHYRSNWPPVLTFGGGTKVEIE | | | | | | |
| | COV2- | 1388 | DIQMTQSPSSLSASVGDRVTITCQASQ | IGHV4-31 | IGHJ4 | IGKV1-33 | IGKJ2 | 2397 | ARGGSGSYSL |
| | 2158 | | DITNYLNWYQQKPGKAPKLLIYDASNL | (Human) | (Human) | (Human) | (Human) | | FDY |
| | | | ETGVPSRFSGSGSGTDFTFTISSLQPED | | | | | | |
| | | | FATYYCQQYDNLYSVHFGQGTKLEIK | | | | | | |
| | COV2- | 1389 | QSALTQPASVSGSPGQSITISCTGTSSD | IGHV3- | IGHJ6 | IGLV2-14 | IGU1 | 2398 | ARSTSGSYYY |
| | 2159 | | VGDYNYVSWYQQHPGKAPKLMIYDV | 303 | (Human) | (Human) | (Human) | | GMDV |
| | | | SNRPSGVSNRFSGSKSGNTASLTISGL | (Human) | | | | | |
| | | | QAEDEAEYYCSSYTSSSTLLYVFGTGTK | | | | | | |
| | | | VTVL | | | | | | |
| | COV2- | 1390 | QSALTQPASVSGSPGQSITISCTGTSSD | IGHV3- | IGHJ6 | IGLV2-14 | IGU1 | 2399 | ARSTSGSYYY |
| | 2160 | | VGDYNYVSWYQQHPGKAPKLMIYDV | 303 | (Human) | (Human) | (Human) | | GMDV |
| | | | SNRPSGVSNRFSGSKSGNTASLTISGL | (Human) | | | | | |
| | | | QAEDEAEYYCSSYTSSSTLLYVFGTGTK | | | | | | |
| | | | VTVL | | | | | | |
| | COV2- | 1391 | DIQLTQSPSFLSASVGDRVTITCRASQG | IGHV3-66 | IGHJ6 | IGKV1-9 | IGKJ5 | 2400 | ARDLVTYGLD |
| | 2165 | | ISNYLAWYQQKPGTAPNLLIYAASTLQ | (Human) | (Human) | (Human) | (Human) | | V |
| | | | SGVPSRFSGSGSGTEFTLTISSLQPEDF | | | | | | |
| | | | ATYYCQLLNSHPLTFGQGTRLEIK | | | | | | |
| | COV2- | 1392 | EIVLTQSPATLSLSPGERATLSCRASQS | IGHV1-69 | IGHJ4 | IGKV3-11 | IGKJ3 | 2401 | ARIGHFDSSG |
| | 2166 | | VSSFLAWYQQKPGQAPRLLIYDASNR | (Human) | (Human) | (Human) | (Human) | | YYLDY |
| | | | PTGIPARFTGSGSGTDFTLTISSLEPED | | | | | | |
| | | | FAVYYCQHRTNWPPLFTFGPGTKVDIK | | | | | | |
| | COV2- | 1393 | SYELTQPPSVSVSPGQTASITCSGDKLG | IGHV3-7 | IGHJ4 | IGLV3-1 | IGU1 | 2402 | ARLSGSSWDF |
| | 2171 | | DKYACWYQQRPGQSPVLVIYQDSKRP | (Human) | (Human) | (Human) | (Human) | | DY |
| | | | SGIPERFSGSNSGNTATLTISGTQAMD | | | | | | |
| | | | EADYYCQAWDSSTGVFGTGTKVTVL | | | | | | |
| | COV2- | 1394 | DIQMTQSPSTLSASVGDRVTITCRASQ | IGHV3-20 | IGHJ6 | IGKV1-5 | IGKJ1 | 2403 | ARRRSSSRYSS |
| | 2173 | | SVSTWLAWYQQKPGKAPNLLIYEASSL | (Human) | (Human) | (Human) | (Human) | | GWYMYYYY |
| | | | ESGVPSRFSGSGSGTEFTLTISSLQPDD | | | | | | MDV |
| | | | FATYYCQQYNTYSGTFGQGTKVEIK | | | | | | |
| | COV2- | 1395 | SYELTQPPSVSVSPGQTASITCSGDKLG | IGHV3-7 | IGHJ4 | IGLV3-1 | IGU1 | 2404 | ARLSGSSWDF |
| | 2175 | | DKYACWYQQRPGQSPVLVIYQDSKRP | (Human) | (Human) | (Human) | (Human) | | DY |
| | | | SGIPERFSGSNSGNTATLTISGTQAMD | | | | | | |
| | | | EADYYCQAWDSSTGVFGTGTKVTVL | | | | | | |
| | COV2- | 1396 | DIQMTQSPSSLSASVGDRVTITCRARQ | IGHV2-5 | IGHJ3 | IGKV1-39 | IGKJ1 | 2405 | AHRLWFRDA |
| | 2177 | | SISNYLNWYQQKPGKAPKLLIYAASSL | (Human) | (Human) | (Human) | (Human) | | FDI |
| | | | HSGVPSRFSGSGSGTDFTLTISSLQPED | | | | | | |
| | | | FATYYCQQTYSTFWTFGQGTNVEIK | | | | | | |
| | COV2- | 1397 | SYELTQPPSVSVSPGQTASITCSGDKLG | IGHV3-7 | IGHJ4 | IGLV3-1 | IGU2 | 2406 | ARVGSSSWYF |
| | 2178 | | DKYACWYQQKPGQSPVVVIYQDSKR | (Human) | (Human) | (Human) | (Human) | | DY |
| | | | PSGIPERFSGSNSGNTATLTISGTQAM | | | | | | |
| | | | DEADYYCQAWDSSTAVFGGGTKLTVL | | | | | | |
| | COV2- | 1398 | QSALTQPASVSGSPGQSITISCTGTSSD | IGHV3- | IGHJ4 | IGLV2-14 | IGU2 | 2407 | ARADTMVRG |
| | 2183 | | VGGYNYVSWYQQHPGKAPKLMIYDV | 303 | (Human) | (Human) | (Human) | | TYFEY |
| | | | SNRPSGVSNRFSGSKSGNTASLTISGL | (Human) | | | | | |
| | | | QAEDEADYCCSSYTSSRAVLFGGGTKL | | | | | | |
| | | | TVL | | | | | | |
| | COV2- | 1399 | SYVLTQPPSVSVAPGKTANITCGGNNI | IGHV3- | IGHJ4 | IGLV3-21 | IGU3 | 2408 | ARGGATNFD |
| | 2187 | | GRKSVHWYQQKSGQAPVLVVYDDSD | 303 | (Human) | (Human) | (Human) | | Y |
| | | | RPSGIPERFSGSNSGNTATLTISRVEAG | (Human) | | | | | |
| | | | DEADYYCQVWDSSSDHPEWVFGGGT | | | | | | |
| | | | KLTVL | | | | | | |
| | COV2- | 1400 | DIQMTQSPSSLSASVGDRVTITCRASQ | IGHV3-9 | IGHJ5 | IGKV1-39 | IGKJ4 | 2409 | TKASRYCSSTI |
| | 2189 | | SISSYLNWYQQKPGKAPKLLIYGASSLQ | (Human) | (Human) | (Human) | (Human) | | CYWNWFDP |
| | | | TGVPSRFSGSGSGTDFTLTIRSLQPEDF | | | | | | |
| | | | ASYYCQQSYSTPTFGGGTKVEIK | | | | | | |
| | COV2- | 1401 | QSALTQPASVSGSPGQSITISCTGTSSD | IGHV3-48 | IGHJ4 | IGLV2-14 | IGU2 | 2410 | AREARSRYFD |
| | 2190 | | IGGYNYVSWYQQHPGKAPKLLIYDVS | (Human) | (Human) | (Human) | (Human) | | WLPSYYFDY |
| | | | NRPSGVSTRFSGSKSGNTASLTISGLQ | | | | | | |
| | | | AEDEADYYCSSYTSSSTHVVFGGGTKL | | | | | | |
| | | | TVL | | | | | | |
| | COV2- | 1402 | DIQMTQSPSTLSASVGDRVTITCRASQ | IGHV3-20 | IGHJ6 | IGKV1-5 | IGKJ1 | 2411 | ARRRSSSRYSS |
| | 2191 | | SVSTWLAWYQQKPGKAPNLLIYEASSL | (Human) | (Human) | (Human) | (Human) | | GWYMYYYY |
| | | | ESGVPSRFSGSGSGTEFTLTISSLQPDD | | | | | | MDV |
| | | | FATYYCQQYNTYSGTFGQGTKVEIK | | | | | | |
| | COV2- | 1403 | DIVMTQSPDFLAVSLGERATISCKSSQS | IGHV3-30 | IGHJ4 | IGKV4-1 | IGKJ4 | 2412 | AKGGDGSG |
| | 2195 | | VLYTPKNKNYLAWYKQKPGQPPKVLIY | (Human) | (Human) | (Human) | (Human) | | WAWDGDNP |
| | | | WASTRESGVPDRFSGSGSGTDFTLIISS | | | | | | PTDY |
| | | | LQAEDAAVYYCQQYYTAPLTFGGGTR | | | | | | |
| | | | VEIK | | | | | | |
| | COV2- | 1404 | EIVLTQSPGTLSLSPGERATLSCRASQS | IGHV1-58 | IGHJ3 | IGKV3-20 | IGKJ1 | 2413 | AAPYCSSISCN |
| | 2196 | | VSSSYLAWYQQKPGQAPRLLIYGASSR | (Human) | (Human) | (Human) | (Human) | | DGFDI |
| | | | ATGIPDRFSGSGSGTDFTLTISRLEPED | | | | | | |
| | | | FAVYYCQHYGSSRGWTFGQGTKVEIK | | | | | | |
| | COV2- | 1405 | EIVLTQSPGTLSLSPGERATLSCRASQS | IGHV5-51 | IGHJ2 | IGKV3-20 | IGKJ5 | 2414 | ARPDYSSGW |
| | 2197 | | VSSNFLAWYQQKPGQAPRLLIYGASS | (Human) | (Human) | (Human) | (Human) | | FSYWYFDL |
| | | | RATGIPDRFSGSGSGTDFTLTISRLEPE | | | | | | |
| | | | DFAVYYCQQYGRSPITFGQGTRLEIK | | | | | | |
| | COV2- | 1406 | SYELTQPPSVSVSPGQTASITCFGDKLG | IGHV1-24 | IGHJ4 | IGLV3-1 | IGU2 | 2415 | ATGFAVFGRA |
| | 2199 | | DKYACWFQQKPGQSPVLIIYQGAKRP | (Human) | (Human) | (Human) | (Human) | | AVPY |
| | | | SGIPERFSGSNSGNTATLTISGTQAMD | | | | | | |
| | | | EADYYCQAWDSSTVVFGGGTKLTVL | | | | | | |
| | COV2- | 1407 | DIQMTQSPSSLSASVGDRVTITCRASQ | IGHV3-30 | IGHJ6 | IGKV1-39 | IGKJ2 | 2416 | ARGSAGNYYY |
| | 2203 | | TITNYLNWYQLKSGRAPKLLIYAASSLQ | (Human) | (Human) | (Human) | (Human) | | GMDV |
| | | | SGVPSRFSGSGSGTDFTLTISSLQPEDF | | | | | | |
| | | | ATYYCQQSYSTPYTFGQGTKLEIK | | | | | | |
| | COV2- | 1408 | QSVLTQPPSVSGAPGQRVTISCTGSSS | IGHV5-51 | IGHJ4 | IGLV1-40 | IGU3 | 2417 | ASALRERGVQ |
| | 2207 | | NIGAGYDVHWYQQLPGTAPKLLIFINS | (Human) | (Human) | (Human) | (Human) | | LWSV |
| | | | NRPSGVPDRFSGSKSGTSASLAITGLQ | | | | | | |
| | | | AEDEADYYCQSYDSSLGALFGGGTKLT | | | | | | |
| | | | VL | | | | | | |
| | COV2- | 1409 | DIQMTQSPSSVSASVGDRVTITCRASQ | IGHV3- | IGHJ4 | IGKV1-12 | IGKJ3 | 2418 | ARDQEWFRE |
| | 2210 | | GISSWLAWYQQKPGKAPKLLIYDASSL | 303 | (Human) | (Human) | (Human) | | LFLFDY |
| | | | QSGVPSRFSGSGSGTDFTLTISSLQPED | (Human) | | | | | |
| | | | FATYYCQQANSFPPTFGPGTKVDIK | | | | | | |
| | COV2- | 1410 | EIVLTQSPAILSLSPGERATLSCRASQS | IGHV3-21 | IGHJ6 | IGKV3-11 | IGKJ1 | 2419 | ARVNGNSN |
| | 2212 | | VSSYLAWYQQKPGQAPRLLIYDTSNRAT | (Human) | (Human) | (Human) | (Human) | | WNFGSYYYYY |
| | | | GIPARFSGSGSGTDFTLTISSLEPEDFA | | | | | | MDV |
| | | | FYYCQQRGNWWTFAQGTKVEIK | | | | | | |
| | COV2- | 1411 | EIVLTQSPATLSLSPGERATLSCRASQS | IGHV1-69 | IGHJ4 | IGKV3-11 | IGKJ3 | 2420 | ARIGHFDSSG |
| | 2214 | | VSSFLAWYQQKPGQAPRLLIYDASNR | (Human) | (Human) | (Human) | (Human) | | YYLDY |
| | | | PTGIPARFTGSGSGTDFTLTISSLEPED | | | | | | |
| | | | FAVYYCQHRTNWPPLFTFGPGTKVDIK | | | | | | |
| | COV2- | 1412 | EIVMTQSPATLSVSPGERATLSCRASQ | IGHV3-21 | IGHJ6 | IGKV3-15 | IGKJ1 | 2421 | ARWLQLRSD |
| | 2215 | | SVSNNLAWYQHKPGQAPRLLIYGAST | (Human) | (Human) | (Human) | (Human) | | YYYFGMDV |
| | | | RATGIPARFSGSGSGTEFTLTISSLQSE | | | | | | |
| | | | DFAVYFCQQCYNWPPWTFGQGTKVE | | | | | | |
| | | | IK | | | | | | |
| | COV2- | 1413 | EIVLTQSPATLSLSPGERATLSCRASQS | IGHV1-69 | IGHJ4 | IGKV3-11 | IGKJ4 | 2422 | ARTSHYDSSG |
| | 2216 | | VSSYLAWYQQKPGQAPRLLIYDASNR | (Human) | (Human) | (Human) | (Human) | | SYFEY |
| | | | ATGIPARFSGSGSGTDFTLTISSLDPED | | | | | | |
| | | | FAVYYCHKRSNWPPSLTFGGGTKVEIK | | | | | | |
| | COV2- | 1414 | DIVMTQSPDSLAVSLGERATINCKSSQ | IGHV3-30 | IGHJ6 | IGKV4-1 | IGKJ1 | 2423 | ARPYTGSYKS |
| | 2218 | | SVLYSSNNKNSLAWYQQKPGQPPKLLI | (Human) | (Human) | (Human) | (Human) | | YMDV |
| | | | YWASTRESGVPDRFSGSGSGTDFTLTI | | | | | | |
| | | | SSLQAEDVAVYYCQQYYSISWTFGQG | | | | | | |
| | | | TKVEIK | | | | | | |
| | COV2- | 1415 | QSALTQPASVSGSPGQSITISCTGTSSD | IGHV4-59 | IGHJ4 | IGLV2-23 | IGU2 | 2424 | ARAPRERLQ |
| | 2222 | | VGSYNLVSWYQQHAGKAPKLMIYEVI | (Human) | (Human) | (Human) | (Human) | | WGEYYFDY |
| | | | KRPSGVSNRFSGSKSGNTASLTISGLQ | | | | | | |
| | | | AEDEADYYCCSYAVSTTYVIFGGGTKLT | | | | | | |
| | | | VL | | | | | | |
| | COV2- | 1416 | DIVMTQSPDSLAVSLGERATINCKSSQ | IGHV3-30 | IGHJ4 | IGKV4-1 | IGKJ1 | 2425 | AKDGSIAAAD |
| | 2224 | | SVLHSSNNKDSLVWYQQKPGQPPKLL | (Human) | (Human) | (Human) | (Human) | | Y |
| | | | IYWASSRESGVPDRFSGSGSGTDFTLTI | | | | | | |
| | | | SSLQAEDVAVYYCQQYYSTPWTFGQG | | | | | | |
| | | | TKVEIK | | | | | | |
| | COV2- | 1417 | QPVLTQPPSASASLGASVTLTCTLSSGY | IGHV5-51 | IGHJ4 | IGLV9-49 | IGU2 | 2426 | ATHRCSGGFC |
| | 2226 | | SNYKVDWYQQRPGKGPRFVMRVGT | (Human) | (Human) | (Human) | (Human) | | YLAY |
| | | | GGIVGSKGDGIPDRFSVLGSGLNRYLTI | | | | | | |
| | | | KNIQEEDESDYHCGADHGSGSNFVFV | | | | | | |
| | | | VFGGGTKLTVL | | | | | | |
| | COV2- | 1418 | DIVMTQSPLSLPVTPGEPASISCRSSQS | IGHV3-33 | IGHJ6 | IGKV2-28 | IGKJ3 | 2427 | ARDQSQGAYI |
| | 2227 | | LLHSNGYNYLDWYLQKPGQSPQFLIYL | (Human) | (Human) | (Human) | (Human) | | LTGYRGYGM |
| | | | GSNRASGVPDRFSGSGSGTDFILKISR | | | | | | DV |
| | | | VEAEDVGVYYCMQALQTPFTFGPGTK | | | | | | |
| | | | VDIK | | | | | | |
| | COV2- | 1419 | EIVMTQSPATLSVSPGERATLSCRASQ | IGHV4-34 | IGHJ6 | IGKV3-15 | IGKJ4 | 2428 | ARPPQAARIH |
| | 2228 | | SVSSNLAWYQQKPGQAPRLLIYGAST | (Human) | (Human) | (Human) | (Human) | | YYYYMDV |
| | | | RATGIPARFSGSGSGTEFTLTISSLQSE | | | | | | |
| | | | DFAVYYCQQYNYWPPLTFGGGTKVEI | | | | | | |
| | | | K | | | | | | |
| | COV2- | 1420 | SYVLTQPPSVSVAPGKTARITCEGNNI | IGHV3-9 | IGHJ6 | IGLV3-21 | IGU2 | 2429 | AKGRGAGYTS |
| | 2231 | | GSKSVHWYQQKPGQAPVLVVYDDSG | (Human) | (Human) | (Human) | (Human) | | YMDV |
| | | | RPSGIPERFSGSNSGNTATLTISRVEAG | | | | | | |
| | | | DEADYFCQVWDSSSDHHVVFGGGTK | | | | | | |
| | | | LTVL | | | | | | |
| | COV2- | 1421 | DIQMTQSPSTLSASVGDRVTITCRASH | IGHV3-53 | IGHJ4 | IGKV1-5 | IGKJ1 | 2430 | ARESTQ |
| | 2235 | | SISSWLAWYQQKPGKAPKLLIYKASSL | (Human) | (Human) | (Human) | (Human) | | |
| | | | ESGVPSRFSGSGSGTEFTLTISSLQPDD | | | | | | |
| | | | FATYYCQQYNTYSQTFGQGTKVEIK | | | | | | |
| | COV2- | 1422 | FYELTQPPSVSVSPGQTAGITCSGDKL | IGHV2-5 | IGHJ4 | IGLV3-1 | IGU3 | 2431 | AHRAVILNFD |
| | 2238 | | GHKYAYWYQQKPGQSPILLIYQDDKR | (Human) | (Human) | (Human) | (Human) | | |
| | | | PSGIPERFSGSNSGTIATLTISGTQPVD | | | | | | |
| | | | EADYYCQAWDNDAGVVFGGGTKLTV | | | | | | |
| | | | L | | | | | | |
| | COV2- | 1423 | DIQMTQSPSSLSASVGDRVTITCRASQ | IGHV3- | IGHJ4 | IGKV1-16 | IGKJ4 | 2432 | ARPRSGSYYA |
| | 2239 | | GISNYLAWFQQKPGKAPKSLIYAASSL | 303 | (Human) | (Human) | (Human) | | YFDY |
| | | | QSGVPSKFSGSGSGTDFTLTISSLQPED | (Human) | | | | | |
| | | | VATYYCQQYNSHPPTFGGGTKVEIK | | | | | | |
| | COV2- | 1424 | QSALTQPPSASGSPGQSVTISCTGTSS | IGHV3-9 | IGHJ4 | IGLV2-8 | IGU2 | 2433 | AKVGYTISRQ |
| | 2240 | | DVGTYNYVSWYQQHPGKAPKLMIYE | (Human) | (Human) | (Human) | (Human) | | WLVGEFDY |
| | | | VSKRPSGVPDRFSGSKSGNTASLTVSG | | | | | | |
| | | | LQAEDEAEYYCSAYAGSNNLVFGGGT | | | | | | |
| | | | KLTVL | | | | | | |
| | COV2- | 1425 | QPVLTQPPSASASLGASVTLTCTLSSGY | IGHV5-51 | IGHJ2 | IGLV9-49 | IGU2 | 2434 | ARRGEAAGI |
| | 2241 | | SNYKVDWYQQRPGKGPRFVMRVGT | (Human) | (Human) | (Human) | (Human) | | WYFDL |
| | | | GGIVGSKGDGIPDRFSVLGSGLNRYLTI | | | | | | |
| | | | KNIQEEDESDYHCGADHGSGSNFEYV | | | | | | |
| | | | VFGGGTKLTVL | | | | | | |
| | COV2- | 1426 | QSALTQPASVSGSHGQSITISCTGTSSD | IGHV4-39 | IGHJ4 | IGLV2-23 | IGU2 | 2435 | ARQSRGYSYA |
| | 2243 | | VGSYNLVSWYQQHPGKAPKLMIYEVS | (Human) | (Human) | (Human) | (Human) | | WSFDY |
| | | | KRPSGVSNRFSGSTSGNTASLTISGLQ | | | | | | |
| | | | AEDEADYYCCSYAGIVLFGGGTKVTVL | | | | | | |
| | COV2- | 1427 | SYELTQPPSVSVSPGQTTRITCSGDALP | IGHV3-33 | IGHJ6 | IGLV3-25 | IGU3 | 2436 | AGSSGEGGLY |
| | 2245 | | KQYGYWYQQKAGQAPILVIYKDSERP | (Human) | (Human) | (Human) | (Human) | | YYYGMDV |
| | | | SGIPERFSGSSSGTTVTLTISGVQAEDE | | | | | | |
| | | | AHYYCQSGDSSGTYVVFGGGTKLTVL | | | | | | |
| | COV2- | 1428 | SYELTQPPSVSVSPGQTARITCSGDALS | IGHV3-30 | IGHJ4 | IGLV3-25 | IGU3 | 2437 | ARDSGGNYG |
| | 2248 | | KQYAYWYQQKPGQAPVLVIYKDSERP | (Human) | (Human) | (Human) | (Human) | | DSYFDY |
| | | | SGIPERFSGSSSGTTVTLTISGVQAEDE | | | | | | |
| | | | ADYYCQSVDRSGTYFNWVFGGGTKLT | | | | | | |
| | | | VL | | | | | | |
| | COV2- | 1429 | DIQMTQSPSSLSASVGDRVTITCRASQ | IGHV3-30 | IGHJ6 | IGKV1-39 | IGKJ2 | 2438 | ARGSAGNYYY |
| | 2250 | | TITNYLNWYQLKSGRAPKLLIYAASSLQ | (Human) | (Human) | (Human) | (Human) | | GMDV |
| | | | SGVPSRFSGSGSGTDFTLTISSLQPEDF | | | | | | |
| | | | ATYYCQQSYSTPYTFGQGTKLEIK | | | | | | |
| | COV2- | 1430 | QTVVTQEPSLTVSPGGTVTLTCASSAG | IGHV2-26 | IGHJ5 | IGLV7-43 | IGU3 | 2439 | ARIVLGASGT |
| | 2251 | | AVTSGYYPNWFQQKPGQAPRALIYST | (Human) | (Human) | (Human) | (Human) | | YPSPGFDP |
| | | | ANKHSWTLARFSGSLLGGKAALTLSG | | | | | | |
| | | | VQPEDEAEYYCLLYYGGAWVFGGGTK | | | | | | |
| | | | LTVL | | | | | | |
| | COV2- | 1431 | NFMLTQPHSVSESPGKTVTISCTGSSG | IGHV3-74 | IGHJ4 | IGLV6-57 | IGU2 | 2440 | AGSPWLRGDI |
| | 2253 | | SIASNYVQWYQQRPGSAPTTVIYEDN | (Human) | (Human) | (Human) | (Human) | | DY |
| | | | QRPSGVPDRFSGSIDSSSNSASLTISGL | | | | | | |
| | | | MTEDEADYYCQSYDGSNHAVVFGGG | | | | | | |
| | | | TKLTVL | | | | | | |
| | COV2- | 1432 | SYVLTQPPSVSVAPGKTARITCGGNNI | IGHV7-4- | IGHJ5 | IGLV3-21 | IGU2 | 2441 | ARDPSYCSST |
| | 2256 | | GSKNVHWYQQKPGQAPVVVVYDDS | 1 | (Human) | (Human) | (Human) | | RCYTVGWFD |
| | | | DRPSGIPERFSGSNSGNTATLTISRVEA | (Human) | | | | | P |
| | | | GDEADYYCQVWDSSSDPVVFGGGTK | | | | | | |
| | | | LTVL | | | | | | |
| | COV2- | 1433 | NIVMTQSPLSLPVTPGEPASISCRSSQS | IGHV4-61 | IGHJ4 | IGKV2-28 | IGKJ1 | 2442 | ARGHVAAWE |
| | 2257 | | LLNSNGLTYLDWYLQKPGQSPQLLIYF | (Human) | (Human) | (Human) | (Human) | | SCYY |
| | | | VSNRASGVSDRFSGSGSGADFTLTISR | | | | | | |
| | | | VEAEDVGVYYCMQALQTPQTFGQGT | | | | | | |
| | | | KVEIK | | | | | | |
| | COV2- | 1434 | EIVLTQSPGTLSLSPGERATLSCGASQS | IGHV3-20 | IGHJ3 | IGKV3-20 | IGKJ4 | 2443 | ARARGPSEQY |
| | 2258 | | ISSSYLAWYQQKPGQAPRLLIYGASSRA | (Human) | (Human) | (Human) | (Human) | | YDLLTGYYDA |
| | | | AGIPDRFSGSGSGTDFTLTISRLEPEDF | | | | | | FDI |
| | | | AVYFCQQYGGSLTFGGGTKVEIK | | | | | | |
| | COV2- | 1435 | SYELTQPPSVSVSPGQTASITCSGDKM | IGHV3-23 | IGHJ4 | IGLV3-1 | IGU2 | 2444 | AQMGPLGST |
| | 2260 | | GDKYACWYQQKPGQSPVVVIYEDNK | (Human) | (Human) | (Human) | (Human) | | SSAADY |
| | | | RPSGIPERFSGSNSGNTATLTISGTQA | | | | | | |
| | | | MDEADYYCQAWDSSLVVFGGGTKLT | | | | | | |
| | | | VL | | | | | | |
| | COV2- | 1436 | QSALTQPPSVSGSPGQSVTISCTGTSS | IGHV1-8 | IGHJ6 | IGLV2-18 | IGU3 | 2445 | AREARYFDWI |
| | 2262 | | DVGSYNRVSWYQQPPGTAPKLMIYE | (Human) | (Human) | (Human) | (Human) | | FEGSDYYYYG |
| | | | VSNRPSGVPDRFSGSKSGNTASLTISG | | | | | | MDV |
| | | | LQAEDEADYYCSSYTSSSLRVFGGGTK | | | | | | |
| | | | LTVL | | | | | | |
| | COV2- | 1437 | DIQLTQSPSFLSASVGDRVTITCRASQG | IGHV3-48 | IGHJ6 | IGKV1-9 | IGKJ1 | 2446 | ARRSYRSSWY |
| | 2263 | | ISSYLAWYQQKPGKAPKLLIYAASTLQS | (Human) | (Human) | (Human) | (Human) | | YYYGMDV |
| | | | GVPSRFSGSGSGTEFTLTISSLQPEDFA | | | | | | |
| | | | TYYCQQLNSYPVTFGQGTKVEIK | | | | | | |
| | COV2- | 1438 | QSALTQPRSVSGSPGQSVTISCTGTSS | IGHV3-49 | IGHJ3 | IGLV2-11 | IGU1 | 2447 | IRDYDFWGG |
| | 2266 | | DVGGYNYVSWYQQHPGKAPKLMIYD | (Human) | (Human) | (Human) | (Human) | | YYYHPLRAFDI |
| | | | VTKRPSGVPERFSGSKSGNTASLTISGL | | | | | | |
| | | | QAEDEADYSCCSYAGSYTYVFGTGTKV | | | | | | |
| | | | TVL | | | | | | |
| | COV2- | 1439 | QSALTQPASVSGSPGQSITISCTGTGSD | IGHV2-5 | IGHJ3 | IGLV2-14 | IGU2 | 2448 | ARHQIVVLFD |
| | 2268 | | VGGSNYVSWYQQHPGKAPKLMIYDV | (Human) | (Human) | (Human) | (Human) | | M |
| | | | SNRPSGVSNRFSGSKSGNTASLTISGL | | | | | | |
| | | | QAEDEADYYCSSYTSSSTLVLFGGGTKL | | | | | | |
| | | | SVL | | | | | | |
| | COV2- | 1440 | EIVLTQSPATLSLSPGERATLSCRASQS | IGHV1-69 | IGHJ4 | IGKV3-11 | IGKJ2 | 2449 | AITYYYDSSGY |
| | 2270 | | VSSFLAWYQQKPGQAPRLLIYDASNR | (Human) | (Human) | (Human) | (Human) | | WWDD |
| | | | ATGIPARFSGSGSGTDFTLTISSLEPED | | | | | | |
| | | | FAVYYCQQRSNWPPSYTFGQGTKLEIK | | | | | | |
| | COV2- | 1441 | QSALTQPASVSGSPGQSITISCTGTSSD | IGHV4-39 | IGHJ6 | IGLV2-14 | IGU3 | 2450 | AGEEVRGVKL |
| | 2273 | | IGGYNYVSWYQQHPGKAPKLMIYDVS | (Human) | (Human) | (Human) | (Human) | | YYYYAMDV |
| | | | NRPSGVSNRFSGSKSGNTASLTISGLQ | | | | | | |
| | | | AEDEADYYCSSYTSISTWVFGGGTKVT | | | | | | |
| | | | VL | | | | | | |
| | COV2- | 1442 | SYELTQPSSVSVSPGQTARITCSGDALP | IGHV3-48 | IGHJ4 | IGLV3-10 | IGU2 | 2451 | ARDRGWNYG |
| | 2274 | | KRYAYWYRQKSGQAPVLVIHEDSKRP | (Human) | (Human) | (Human) | (Human) | | LDY |
| | | | SGIPERFSGSTSGTMATLTITGAQLEDE | | | | | | |
| | | | ADYYCFSMDSSGDLRVFGGGTKLTVL | | | | | | |
| | COV2- | 1443 | DIQMTQSPSSLSASVGDRVTITCRASQ | IGHV3-30 | IGHJ4 | IGKV1-39 | IGKJ4 | 2452 | ARDVPTTVTA |
| | 2277 | | SISTYLNWYQQKPGKAPKLLIYAASSLQ | (Human) | (Human) | (Human) | (Human) | | FTVFTY |
| | | | SGVPSRFSGSGSGTDFTLTISSLQPEDY | | | | | | |
| | | | ATYYCQQSYSTPGLTFGGGTKVEIK | | | | | | |
| | COV2- | 1444 | QSALTQPASVSGSPGQSITISCTGTSSD | IGHV3- | IGHJ4 | IGLV2-14 | IGU2 | 2453 | ARVPVMVRG |
| | 2281 | | VVGYNYVSWYQQHPGKAPKLMIYDV | 303 | (Human) | (Human) | (Human) | | VYFDY |
| | | | RNRPSGVSNRFSGSKSGNTASLTISGL | (Human) | | | | | |
| | | | QAEDEADYFCISYTSSRTLVFGGGTKLT | | | | | | |
| | | | VL | | | | | | |
| | COV2- | 1445 | SYVLTQPPSVSVAPGKTARITCGGNNI | IGHV1-8 | IGHJ6 | IGLV3-21 | IGU2 | 2454 | ARGYGLTYY |
| | 2287 | | GSKNVHWYQQKPGQAPVLVVYDDS | (Human) | (Human) | (Human) | (Human) | | MDV |
| | | | DRPSGIPERFSGSNSGNTATLTISRVEA | | | | | | |
| | | | GDEADYYCQVWDSSYYHPVVFGGGT | | | | | | |
| | | | KLTVL | | | | | | |
| | COV2- | 1446 | QSVLTQPPSASGTPGQRVTISCSGSNS | IGHV1-8 | IGHJ4 | IGLV1-44 | IGU2 | 2455 | ARMRSGWPT |
| | 2290 | | NIGSYTVNWYQQFPGTAPKLLIYDNN | (Human) | (Human) | (Human) | (Human) | | HGRPDDF |
| | | | QRTSGVPDRFSGSKSGTSASLAISGLQ | | | | | | |
| | | | SEDEANYYCLVWDDSLNGLVFGGGTK | | | | | | |
| | | | LTVL | | | | | | |
| | COV2- | 1447 | QSVLTQPPSVSAAPGQKVTISCSGSSS | IGHV3-23 | IGHJ4 | IGLV1-51 | IGU2 | 2456 | AKNERITMLV |
| | 2293 | | NIGNNYISWYQQLPGTAPKLLIYDNNK | (Human) | (Human) | (Human) | (Human) | | VVTLFDY |
| | | | RPSGIPDRFSGSKSGTSATLGITGLQTG | | | | | | |
| | | | DEADYYCGTWDSSLSAVVFGGGTKLT | | | | | | |
| | | | VL | | | | | | |
| | COV2- | 1448 | DIQMTQSPSTLSASVGDRVTITCRASQ | IGHV3-66 | IGHJ6 | IGKV1-5 | IGKJ1 | 2457 | ARDPSAYYDIL |
| | 2296 | | SINSWLAWYQQKPGKAPKLLIYKASSL | (Human) | (Human) | (Human) | (Human) | | TGYSGDV |
| | | | ESGVPSRFSGSGSGTEFTLTISSLQPDD | | | | | | |
| | | | FATYYCQQYSIYSWTFGQGTKVEIK | | | | | | |
| | COV2- | 1449 | DIQMTQSPSSLSACVGDRVTITCRASQ | IGHV3- | IGHJ4 | IGKV1-39 | IGKJ1 | 2458 | ARDIDSGYDP |
| | 2299 | | SISSYLNWYQQKPGKGPKLLIYAASSLQ | 303 | (Human) | (Human) | (Human) | | TPVFDY |
| | | | SGVPSRFSGSGSGTDFTLTISSLQPEDF | (Human) | | | | | |
| | | | ATYYCQQSYSTPWTFGQGTKVEIK | | | | | | |
| | COV2- | 1450 | DIQMTQSPSSVSASVGDRVTITCRASQ | IGHV4-4 | IGHJ4 | IGKV1-12 | IGKJ4 | 2459 | ASRWGDYFD |
| | 2300 | | GISSWLAWYQQKPGKAPKLLIYAASSL | (Human) | (Human) | (Human) | (Human) | | SSGAYDS |
| | | | QRGVPSRFSGSGSGTDFTLTISSLQPE | | | | | | |
| | | | DFATYYCQQANSLPLTFGGGTKVEVK | | | | | | |
| | COV2- | 1451 | QSVLTQPPSASGTPGQRVTISCSGSSS | IGHV3-21 | IGHJ4 | IGLV1-44 | IGU2 | 2460 | ARDPVWVDG |
| | 2304 | | NIESNSVNWYQQLPGTAPKLLIYSNN | (Human) | (Human) | (Human) | (Human) | | ELLSGGIPFDY |
| | | | QRPSGVPDRFSGSKSGTSASLAISGLQ | | | | | | |
| | | | SEDEADYYCAAWDDSLNGVVFGGGT | | | | | | |
| | | | KLTVL | | | | | | |
| | COV2- | 1452 | QSVLTQPPSASGTPGQRVTISCSGSTS | IGHV4-39 | IGHJ6 | IGLV1-44 | IGU3 | 2461 | AILWRGSSW |
| | 2305 | | NIGSNTVNWYQQLPGTAPKLLIYSNN | (Human) | (Human) | (Human) | (Human) | | ADRHYYYYSM |
| | | | QRPSGVPDRFSGSKSGPSASLAISGLQ | | | | | | DV |
| | | | SVDEADYYCAAWDDSLNGWVFGGG | | | | | | |
| | | | TKLTVL | | | | | | |
| | COV2- | 1453 | SYELTQSPSVSVSPGQTASITCSGDKLG | IGHV3- | IGHJ5 | IGLV3-1 | IGU3 | 2462 | ARDLGRGLDP |
| | 2307 | | DKYACWYQQRPGQSPVLVIYQDSQR | 303 | (Human) | (Human) | (Human) | | |
| | | | PSGIPERFSGSNSGNTATLTISGTQAM | (Human) | | | | | |
| | | | DEADYYCQAWDSSTAVFGGGTKLTVL | | | | | | |
| | COV2- | 1454 | DIVMTQSPATLSVSPGERAILSCRASQ | IGHV3-23 | IGHJ3 | IGKV3-15 | IGKJ1 | 2463 | VKGLFDWFPL |
| | 2308 | | SVRSNLAWYQQKPGQAPRLLISGAST | (Human) | (Human) | (Human) | (Human) | | |
| | | | RATAIPARFSGSGSGTEFTLTITSLQSE | | | | | | |
| | | | DCAVYYCHQYNNWPQTFGQGTKVEIK | | | | | | |
| | COV2- | 1455 | DIQMTQSPSSLSASVGDRVTITCRASQ | IGHV4-61 | IGHJ6 | IGKV1-39 | IGKJ4 | 2464 | ATGYIGTYYYY |
| | 2310 | | SISTYLNWYQQKPGKAPKLLIYAASSLQ | (Human) | (Human) | (Human) | (Human) | | MDV |
| | | | SGVPSRFSGSGSGTDFTLTISSLQPEDF | | | | | | |
| | | | ATYHCQQSYSTLTFGGGTKVEIK | | | | | | |
| | COV2- | 1456 | EIVLTQSPATLSLSPGERATLSCRASQS | IGHV3-9 | IGHJ4 | IGKV3-11 | IGKJ4 | 2465 | AKVSSITSLLG |
| | 2313 | | VSSYLAWYQQKPGQAPRLLIYDASNR | (Human) | (Human) | (Human) | (Human) | | YYFDS |
| | | | ATGIPARFSGSGSGTDFTLTISSLEPED | | | | | | |
| | | | FAVYYCQHRSNWPPRLTFGGGTKVEIK | | | | | | |
| | COV2- | 1457 | DIQMTQSPSSLSASVGDRVTITCRASQ | IGHV2-70 | IGHJ4 | IGKV1-39 | IGKJ5 | 2466 | ARGVVTYDY |
| | 2318 | | SIAGYLNWYQQKPGKAPKLLIYGTTSL | (Human) | (Human) | (Human) | (Human) | | |
| | | | QSGVPVRFSGSGSGTDFTLTISSLQPE | | | | | | |
| | | | DFATYYCQQSYSTPGTFGQGTRLEIK | | | | | | |
| | COV2- | 1458 | EIVLTQSPGTLSLSPGERATLSCRASQS | IGHV3-30 | IGHJ5 | IGKV3-20 | IGKJ2 | 2467 | ARGDGYRSQ |
| | 2322 | | VSSSYLAWYQQKPGQAPRLLIYGASSR | (Human) | (Human) | (Human) | (Human) | | FDP |
| | | | ATGIPDRFSGSGSGTDFTLTISRLEPED | | | | | | |
| | | | FAVYYCQQYGSSYTFGQGTKLEIK | | | | | | |
| | COV2- | 1459 | DIQMTQSPSSLSASVGDRVTITCRARQ | IGHV2-5 | IGHJ3 | IGKV1-39 | IGKJ1 | 2468 | AHRLWFRDA |
| | 2325 | | SISNYLNWYQQKPGKAPKLLIYAASSL | (Human) | (Human) | (Human) | (Human) | | FDI |
| | | | HSGVPSRFSGSGSGTDFTLTISSLQPED | | | | | | |
| | | | FATYYCQQTYSTFWTFGQGTNVEIK | | | | | | |
| | COV2- | 1460 | QSVLTQPPSASGTPGQRVTISCSGSSS | IGHV3-23 | IGHJ6 | IGLV1-47 | IGU3 | 2469 | ARVEGDWLL |
| | 2329 | | NIGSNYVYWYQQLPGTAPKLLISRNN | (Human) | (Human) | (Human) | (Human) | | GGPYYHYYG |
| | | | QRPSGVPDRFSGSKSGTSASLAISGLRS | | | | | | MDV |
| | | | EDEADYYCAAWDDSLSSWVFGGGTK | | | | | | |
| | | | LTVL | | | | | | |
| | COV2- | 1461 | NFMLTQPHSVSESPGKTVTISCTGSSG | IGHV4-39 | IGHJ5 | IGLV6-57 | IGU2 | 2470 | ARILVIFTLNW |
| | 2331 | | SIASNYVQWYQQRPGSAPTTVIYEDN | (Human) | (Human) | (Human) | (Human) | | FDP |
| | | | QRPSGVPDRFSGSIDSSSNSASLTISGL | | | | | | |
| | | | KTEDEADYYCQSYDSGNPIFGGGTKLT | | | | | | |
| | | | VL | | | | | | |
| | COV2- | 1462 | SYELTQPPSVSVSPGQTASITCSGDKLG | IGHV3-7 | IGHJ4 | IGLV3-1 | IGU3 | 2471 | ARLGGSSWH |
| | 2333 | | DKYSSWYQQKPGQSPVLVIYEDTKRPS | (Human) | (Human) | (Human) | (Human) | | FDY |
| | | | GIPERFSGSNSGNTATLTISGTQAMDE | | | | | | |
| | | | ADYYCQAWSSSTAVFGGGTKLTVL | | | | | | |
| | COV2- | 1463 | QSALTQPASVSGSPGQSITISCTGTSSD | IGHV4-39 | IGHJ6 | IGLV2-14 | IGU2 | 2472 | ARHDGSGEM |
| | 2335 | | VGGYNYVSWYQQHPGKAPKLMIYYV | (Human) | (Human) | (Human) | (Human) | | DTITWGPIYYY |
| | | | SNRPSGVSNRFSGSKSGNTASLTISGL | | | | | | MDV |
| | | | QAEDEADYYCSSYTSSTLNVLFGGGTK | | | | | | |
| | | | LTVL | | | | | | |
| | COV2- | 1464 | DIQMTQSPSSLSASVGDRVTITCRARQ | IGHV2-5 | IGHJ3 | IGKV1-39 | IGKJ5 | 2473 | AHRLWFRDA |
| | 2337 | | SISNYLNWYQQKPGKAPKLLIYAASSL | (Human) | (Human) | (Human) | (Human) | | FDI |
| | | | HSGVPSRFSGSGSGTDFTLTISSLQPED | | | | | | |
| | | | FATYYCQQSYSTPGTFGQGTRLEIK | | | | | | |
| | COV2- | 1465 | QSVLTQPPSVSGAPGQRVTISCTGSSS | IGHV1-3 | IGHJ5 | IGLV1-40 | IGU1 | 2474 | AMGPSAFSW |
| | 2340 | | NIGAGYDVHWYQHLPGTAPKLLIYGN | (Human) | (Human) | (Human) | (Human) | | LDP |
| | | | SNRPSGVPDRFSGSKSGTSASLAITGL | | | | | | |
| | | | QAEDEADYYCQSYDSSLSGWSVFGTG | | | | | | |
| | | | TKVTVL | | | | | | |
| | COV2- | 1466 | QSALTQPASVSGSPGQSITISCTGTSSD | IGHV3- | IGHJ6 | IGLV2-14 | IGU1 | 2475 | ARSTSGSYYY |
| | 2341 | | VGDYNYVSWYQQHPGKAPKLMIYDV | 303 | (Human) | (Human) | (Human) | | GMDV |
| | | | SNRPSGVSNRFSGSKSGNTASLTISGL | (Human) | | | | | |
| | | | QAEDEAEYYCSSYTSSSTLLYVFGTGTK | | | | | | |
| | | | VTVL | | | | | | |
| | COV2- | 1467 | DIQMTQSPSSLSASVGDRVTITCRASQ | IGHV2-5 | IGHJ4 | IGKV1-39 | IGKJ2 | 2476 | AHRPPSYHG |
| | 2342 | | SISIYLNWYQQKPGKAPKLLIYAASTLQ | (Human) | (Human) | (Human) | (Human) | | WCYFDY |
| | | | SGVPSRFSGSGSGTDFTLTISSLQPEDF | | | | | | |
| | | | ATYYCQQSYSTHMSTFGQGTKLEIK | | | | | | |
| | COV2- | 1468 | DIVVTQTPLSLSVTPGQPASISCKSSET | IGHV1-18 | IGHJ4 | IGKV2D-29 | IGKJ5 | 2477 | ARVQRRRLDY |
| | 2343 | | LLHSDGKTYLSWYLQKPGQPPQLLIYEV | (Human) | (Human) | (Human) | (Human) | | |
| | | | SNRFSGVPDRFSGSGSGTDFTLKIGRV | | | | | | |
| | | | EAEDVGLYYCMQSIQLAFGQGTRLEIE | | | | | | |
| | COV2- | 1469 | EIVMTQSPATLSVSPGERATLSCRASQ | IGHV4-34 | IGHJ6 | IGKV3-15 | IGKJ4 | 2478 | ARPPQAARIH |
| | 2346 | | SVSSNLAWYQQKPGQAPRLLIYGAST | (Human) | (Human) | (Human) | (Human) | | YYYYMDV |
| | | | RATGIPARFSGSGSGTEFTLTISSLQSE | | | | | | |
| | | | DFAVYYCQQYNYWPPLTFGGGTKVEI | | | | | | |
| | | | K | | | | | | |
| | COV2- | 1470 | SSELTQDPAVSVALGQTVTITCQGDSL | IGHV5-51 | IGHJ4 | IGLV3-19 | IGU2 | 2479 | ARRFYGPSSF |
| | 2351 | | RSHYASWYKVKPGQAPLLVIYGRNNR | (Human) | (Human) | (Human) | (Human) | | DY |
| | | | PSGIPDRFSGSRSGNTASLTITGAQVD | | | | | | |
| | | | DEADYYCSSGDSSTDHHVVFAGGTKL | | | | | | |
| | | | AW | | | | | | |
| | COV2- | 1471 | DMEVTQFPSSLSASIGDRVTITCRATQ | IGHV1-46 | IGHJ4 | IGKV1-39 | IGKJ4 | 2480 | ARGGEWRIV |
| | 2352 | | SISTYLNWYQQKPGKAPNLLIYSASHL | (Human) | (Human) | (Human) | (Human) | | PGGRDYFDY |
| | | | QRGVPARFSGSGSGTEFTLTITTLQPE | | | | | | |
| | | | DVGTYYCHQSYGVPITFGGGTKVDIK | | | | | | |
| | COV2- | 1472 | EIVMTQSPATLSVSPGERATLSCRASQ | IGHV5-51 | IGHJ5 | IGKV3-15 | IGKJ2 | 2481 | ARLGVSKYCS |
| | 2353 | | SVSSNLAWYQQKPGQAPRLLIYGAST | (Human) | (Human) | (Human) | (Human) | | GGRCLSGGS |
| | | | RATGIPARFSGSGSGTEFTLTISSLQSE | | | | | | NWFDP |
| | | | DFAVYYCQQYNNWPPMYTFGQGTKL | | | | | | |
| | | | EIK | | | | | | |
| | COV2- | 1473 | NFMLTQPHSVSESPGKTVTISCTGSSG | IGHV3-53 | IGHJ3 | IGLV6-57 | IGU2 | 2482 | ASSSWLRGAF |
| | 2354 | | SIASNYVQWYQQRPGSAPTTVIYEDN | (Human) | (Human) | (Human) | (Human) | | DI |
| | | | QRPSGVPDRFSGSIDSSSNSASLTISGL | | | | | | |
| | | | KTEDEADYYCQSYDSSKYVVFGGGTKL | | | | | | |
| | | | TVL | | | | | | |
| | COV2- | 1474 | EIVLTQSPGTLSLSPGERATLSCRASQS | IGHV1-58 | IGHJ3 | IGKV3-20 | IGKJ1 | 2483 | AAPHCNRTSC |
| | 2355 | | VSSSYLGWYQQKPGQAPRLLIYGASSR | (Human) | (Human) | (Human) | (Human) | | YDAFDL |
| | | | ATGIPDRFSGSGSGTDFTLTISRLEPED | | | | | | |
| | | | FAVYYCQQYGSSPWTFGQGTKVEIK | | | | | | |
| | COV2- | 1475 | DIVMTQSPLSLPVTPGEPASISCRSSQS | IGHV3-49 | IGHJ6 | IGKV2-28 | IGKJ2 | 2484 | SRVRGSFYGS |
| | 2357 | | LLHSNGYNYLDWYLQKPGQSPQLLIYL | (Human) | (Human) | (Human) | (Human) | | VGKNYGMDV |
| | | | GSTRASGVPDRFSGSGSGTDFTLKISR | | | | | | |
| | | | VEAEDVGVYYCMQALQTPLYTFGQGT | | | | | | |
| | | | KLEIK | | | | | | |
| | COV2- | 1476 | DIQMTQSPSSLSACVGDRVTITCRASQ | IGHV3- | IGHJ4 | IGKV1-39 | IGKJ5 | 2485 | ARDIDSGYDP |
| | 2358 | | SISSYLNWYQQKPGKGPKLLIYAASSLQ | 303 | (Human) | (Human) | (Human) | | TPVFDY |
| | | | SGVPSRFSGSGSGTDFTLTISSLQPEDF | (Human) | | | | | |
| | | | ATYYCQQSYSSLSITFGQGTRLEIK | | | | | | |
| | COV2- | 1477 | EIVLTQSPATLSLSPGERATLSCRASQS | IGHV1-69 | IGHJ4 | IGKV3-11 | IGKJ4 | 2486 | ARTSHYDSSG |
| | 2367 | | VSSYLAWYQQKPGQAPRLLIYDASNR | (Human) | (Human) | (Human) | (Human) | | SYFEY |
| | | | ATGIPARFSGSGSGTDFTLTISSLDPED | | | | | | |
| | | | FAVYYCHKRSNWPPSLTFGGGTKVEIK | | | | | | |
| | COV2- | 1478 | SNELTQPPSVSVSPGQTARITCSGDAL | IGHV5-51 | IGHJ6 | IGLV3-25 | IGU2 | 2487 | ARRRGGIGIE |
| | 2368 | | PKQYAYWYQQKPGQAPVLVIYKDTER | (Human) | (Human) | (Human) | (Human) | | YGMDV |
| | | | PSGIPERFSGSSSGTTVTLTISGVQAED | | | | | | |
| | | | EADYYCQSTASSGTYVVFGGGTKLTVL | | | | | | |
| | COV2- | 1479 | DIQMTQSPSTLSASVGDRVTITCRASQ | IGHV3-30 | IGHJ4 | IGKV1-5 | IGKJ1 | 2488 | AKDFGGDNT |
| | 2369 | | SISSWLAWYQQKPGKAPKLLIYKASSL | (Human) | (Human) | (Human) | (Human) | | AMVEYFFDF |
| | | | ESGVPSRFSGSGSGTEFTLTISSLQPDD | | | | | | |
| | | | FATYYCQQYNSYSPTFGQGTKVEIK | | | | | | |
| | COV2- | 1480 | NFMLTQPHSVSESPGKTVTFSCTGSSG | IGHV4-61 | IGHJ4 | IGLV6-57 | IGU2 | 2489 | AGSPVPPTIV |
| | 2370 | | SIASNYVQWYQQRPGSAPTTVIYEDN | (Human) | (Human) | (Human) | (Human) | | GASY |
| | | | QRPSGVPDRFSGSIDSSSNSASLTISGL | | | | | | |
| | | | KTEDEADYYCQSYDGINRWLVFGGGT | | | | | | |
| | | | KLTVL | | | | | | |
| | COV2- | 1481 | EIVMTQSPATLSVSPGERATLSCRASQ | IGHV3-30 | IGHJ4 | IGKV3-15 | IGKJ1 | 2490 | AKGRGNYLTF |
| | 2371 | | SVSSNLAWYQQKPGQAPRLLIYGAST | (Human) | (Human) | (Human) | (Human) | | FDS |
| | | | RATGIPARFSGSGSGTEFTLTISSLQSE | | | | | | |
| | | | DFAVYYCQQYNNWPGTFGQGTKVEI | | | | | | |
| | | | K | | | | | | |
| | COV2- | 1482 | EIVLTQSPGTLSLSPGERATLSCRASQS | IGHV3-66 | IGHJ6 | IGKV3-20 | IGKJ2 | 2491 | ARDPGSRYSG |
| | 2373 | | VSSSYLAWYQQKPGQAPRLLIYGASSR | (Human) | (Human) | (Human) | (Human) | | GWYDYYYAM |
| | | | ATGIPDRFSGSGSGTDFTLTISRLEPED | | | | | | DV |
| | | | FAVYYCQQYGSSPPYTFGQGTKLEIK | | | | | | |
| | COV2- | 1483 | QPVLTQPPSASASLGASVTLTCTLSSGY | IGHV5-51 | IGHJ4 | IGLV9-49 | IGU2 | 2492 | ARRDTDFDY |
| | 2378 | | SNYKVDWFQQRPGKGPRFVMRVGT | (Human) | (Human) | (Human) | (Human) | | |
| | | | GGIVGSKGDGIPDRFSVLGSGLNRYLTI | | | | | | |
| | | | KNIQEEDESDYHCGADHGSGSNFVYV | | | | | | |
| | | | VFGGGTKLTVL | | | | | | |
| | COV2- | 1484 | EVVLTQSPGTLSLSPGERATLSCRASQS | IGHV1-58 | IGHJ3 | IGKV3-20 | IGKJ1 | 2493 | AAPYCSRTSC |
| | 2381 | | VSSSYLAWYQQKPGQAPRLLIYGASSR | (Human) | (Human) | (Human) | (Human) | | HDAFDI |
| | | | ATGIPDRFSGSGSGTDFTLTISRLEPED | | | | | | |
| | | | FAVYHCQHFGSSSQWTFGQGTKVEIK | | | | | | |
| | COV2- | 1485 | QTVVTQEPSLTVSPGGTVTLTCASSTG | IGHV3-30 | IGHJ4 | IGLV7-43 | IGU3 | 2494 | AKDLPPYASG |
| | 2382 | | AVTSGYFPNWFQQKPGQAPRALIFST | (Human) | (Human) | (Human) | (Human) | | WYEGGFDY |
| | | | NNRHSWTPARFSGSLLGDKAALTLSG | | | | | | |
| | | | VQPEDEAEYYCLLYYGGPWVFGGGTK | | | | | | |
| | | | LTVL | | | | | | |
| | COV2- | 1486 | EIVMTQSPGTLSVSPGERATLSCRASQ | IGHV3-30 | IGHJ4 | IGKV3-15 | IGKJ1 | 2495 | AQGRGGYYS |
| | 2383 | | TLSSNLAWYQQKPGQAPRLLIYGASTR | (Human) | (Human) | (Human) | (Human) | | PFDD |
| | | | ATGIPARFSGSGSGTEFTLTISSLQSED | | | | | | |
| | | | FAVYHCQQYNNWPLAFGQGTKVEIK | | | | | | |
| | COV2- | 1487 | QSALTQPASVSGSPGQSITISCTGTSSD | IGHV2-26 | IGHJ2 | IGLV2-23 | IGU2 | 2496 | ARTTWGTWI |
| | 2384 | | IGSYNLVSWYQQYPGKAPKLMIYEVSK | (Human) | (Human) | (Human) | (Human) | | QAWYFDI |
| | | | RPSGVSNRFSGSKSGNTASLTISGLQA | | | | | | |
| | | | EDEADYYCCSYAGGNTFVVFGGGTKL | | | | | | |
| | | | TVL | | | | | | |
| | COV2- | 1488 | EIVMTQSPATLSVSPGERATLSCRASQ | IGHV3-33 | IGHJ5 | IGKV3-15 | IGKJ1 | 2497 | AREGDFWSG |
| | 2386 | | SVSSNLAWYQQKPGQAPRLLIYGAST | (Human) | (Human) | (Human) | (Human) | | YYTGWFDP |
| | | | RATGIPARFSGSGSGTEFTLTISSLQSE | | | | | | |
| | | | DFAVYYCQQYNNWPRTFGQGNRVEI | | | | | | |
| | | | N | | | | | | |
| | COV2- | 1489 | QSALTQPASVSGSPGQSITISCTGTSSD | IGHV3-30 | IGHJ4 | IGLV2-14 | IGU2 | 2498 | AKDLTIVVIPA |
| | 2387 | | VGGYNYVSWYQQHPGKAPKLMIYDV | (Human) | (Human) | (Human) | (Human) | | APNFDY |
| | | | SNRPSGVSNRFSGSRSGNTASLTISGL | | | | | | |
| | | | QAEDEADYYCSSYTSSSTPVVFGGGTK | | | | | | |
| | | | LTVL | | | | | | |
| | COV2- | 1490 | DIVMTQSPLSLPVTPGEPASISCRSSQS | IGHV3-23 | IGHJ6 | IGKV2-28 | IGKJ1 | 2499 | AKDQARVQD |
| | 2388 | | LLHSNGYNYLDWYLQKPGQSPHLLIYL | (Human) | (Human) | (Human) | (Human) | | YIWGSYRSYG |
| | | | GSNRASGVPDRFSGSGSGTDFTLKISR | | | | | | MDV |
| | | | VEAEDVGVYYCMQALQTPRTFGQGT | | | | | | |
| | | | KVEIK | | | | | | |
| | COV2- | 1491 | DIQMTQSPSSLSASVGDRVTITCRASQ | IGHV3-9 | IGHJ6 | IGKV1-39 | IGKJ1 | 2500 | AKDIIRQGED |
| | 2389 | | NIASYLNWYQQKPGKAPKLLIYAASSL | (Human) | (Human) | (Human) | (Human) | | GMDV |
| | | | QSGVPSRFSGSGSGTDFTLTISSLQPEE | | | | | | |
| | | | FATYYCQQSYSTPWTFGQGTKVEIK | | | | | | |
| | COV2- | 1492 | QSVLTQAPSASGTPGQRVTISCSGSNS | IGHV1-8 | IGHJ4 | IGLV1-44 | IGU2 | 2501 | ARMRSGWPT |
| | 2391 | | NIGSYTINWYQQLPGTAPKLLIYGNDQ | (Human) | (Human) | (Human) | (Human) | | HGRPDDF |
| | | | RTSGVPDRFSGSKFGTSASLAISGLQSE | | | | | | |
| | | | DENNYYCAVWDDSLNGLVFGGGTKL | | | | | | |
| | | | TVL | | | | | | |
| | COV2- | 1493 | EIVMTQSPATLSVSPGERATLSCRASQ | IGHV3-21 | IGHJ6 | IGKV3-15 | IGKJ1 | 2502 | ARWLQLRSD |
| | 2394 | | SVSNNLAWYQHKPGQAPRLLIYGAST | (Human) | (Human) | (Human) | (Human) | | YYYFGMDV |
| | | | RATGIPARFSGSGSGTEFTLTISSLQSE | | | | | | |
| | | | DFAVYFCQQCYNWPPWTFGQGTKVE | | | | | | |
| | | | IK | | | | | | |
| | COV2- | 1494 | SYELTQPPSVSVSPGQTASITCSGDKLG | IGHV3-7 | IGHJ4 | IGLV3-1 | IGU1 | 2503 | ARLSGSSWDF |
| | 2397 | | DKYACWYQQRPGQSPVLVIYQDSKRP | (Human) | (Human) | (Human) | (Human) | | DY |
| | | | SGIPERFSGSNSGNTATLTISGTQAMD | | | | | | |
| | | | EADYYCQAWDSSTGVFGTGTKVTVL | | | | | | |
| | COV2- | 1495 | DIQMTQSPSSLSASVGDRVTITCQASQ | IGHV3-66 | IGHJ4 | IGKV1-33 | IGKJ1 | 2504 | ARDYRDWI |
| | 2399 | | DISKYLNWYQQKPGKAPKLLIYDASNL | (Human) | (Human) | (Human) | (Human) | | |
| | | | ERGVPSRFSGSGSGTDFTFTISSLQAED | | | | | | |
| | | | IATYYCQQYHNLPRTFGQGTKVEIK | | | | | | |
| | COV2- | 1496 | DIQLTQSPSFLSASVRDRVTITCRASQG | IGHV2-5 | IGHJ4 | IGKV1-9 | IGKJ3 | 2505 | AHNRFQYCSS |
| | 2400 | | ISSYLAWYQQKPGKAPKLLIYAASTLQS | (Human) | (Human) | (Human) | (Human) | | TTCYTLLPFDY |
| | | | GVPSRFSGSGSGTEFTLTISSLQPEDFA | | | | | | |
| | | | TYYCQQLNSYPFTFGPGTKVDIK | | | | | | |
| | COV2- | 1497 | DIVMTQSPDSLAVSLGERATINCKSSQ | IGHV3-30 | IGHJ6 | IGKV4-1 | IGKJ1 | 2506 | ARPYTGSYKS |
| | 2401 | | SVLYSSNNKNSLAWYQQKPGQPPKLLI | (Human) | (Human) | (Human) | (Human) | | YMDV |
| | | | YWASTRESGVPDRFSGSGSGTDFTLTI | | | | | | |
| | | | SSLQAEDVAVYYCQQYYSISWTFGQG | | | | | | |
| | | | TKVEIK | | | | | | |
| | COV2- | 1498 | QSALTQPASVSGSPGQSITISCTGTSSD | IGHV3-30 | IGHJ5 | IGLV2-14 | IGU1 | 2507 | ARGDGDVYN |
| | 2403 | | VGGYNYVSWYQQHPGKAPKLMIYDV | (Human) | (Human) | (Human) | (Human) | | FLLVRNWFDP |
| | | | GNRPSGVSNRFSGSKSGNTASLTISGL | | | | | | |
| | | | QAEDEADYYCSSYTSSSTLYVFGTGTKV | | | | | | |
| | | | TVL | | | | | | |
| | COV2- | 1499 | DIQMTQSPSSLSASVGDRVTFTCRASQ | IGHV4-39 | IGHJ4 | IGKV1-27 | IGKJ1 | 2508 | ASLWFGDLYS |
| | 2405 | | GISNYLAWYQQKPGKVPKLLIYAASTL | (Human) | (Human) | (Human) | (Human) | | FDY |
| | | | QSGVPSRFSGIGSGTDFTLTISSLQPED | | | | | | |
| | | | VATYYCQKYNSAPWTFGQGTKVEIK | | | | | | |
| | COV2- | 1500 | SYELTQPPSVSVSPGQTASITCSGDKLG | IGHV3-7 | IGHJ4 | IGLV3-1 | IGU2 | 2509 | ARLGRSSWN |
| | 2406 | | DKFVCWYQQKPGQSPVLVIYQDNKR | (Human) | (Human) | (Human) | (Human) | | FDY |
| | | | PSGIPERFSGSNSGNTATLTISGTQALD | | | | | | |
| | | | EADYYCQAWDSSTGVFGGGTKLTVL | | | | | | |
| | COV2- | 1501 | DIQLTQSPSFLSASVGDRVTITCRASQG | IGHV4-61 | IGHJ6 | IGKV1-9 | IGKJ5 | 2510 | ARVGGISPYY |
| | 2408 | | ISSYLAWYQQKPGKAPKLLMYAASTL | (Human) | (Human) | (Human) | (Human) | | YYYYMDV |
| | | | QSGVPSRFSGSGSGTEFTLTINTLQPE | | | | | | |
| | | | DFATYYCQQLNSYPITFGQGTRLEIK | | | | | | |
| | COV2- | 1502 | QSVLTQPPSASGTPGQRVTFSCSGSNS | IGHV1-8 | IGHJ4 | IGLV1-44 | IGU3 | 2511 | ARMRSGWPT |
| | 2413 | | NIGSYTVNWYQQLPGTAPKLLIYDNN | (Human) | (Human) | (Human) | (Human) | | HGRPDDY |
| | | | QRPSGVPDRFSGSKSGTSASLAISGLQ | | | | | | |
| | | | SEDEADYYCAVWDDSLNGLVFGGGT | | | | | | |
| | | | KLTVL | | | | | | |
| | COV2- | 1503 | QSVLTQPPSVSEAPRQRVTISCSGSSS | IGHV4-31 | IGHJ4 | IGLV1-36 | IGU2 | 2512 | ASAKLVATISY |
| | 2416 | | NIGNNAVNWYQQLPGKAPKLLIYYDD | (Human) | (Human) | (Human) | (Human) | | FDY |
| | | | LLPSGVSDRFSGSKSGTSASLAISGLQS | | | | | | |
| | | | EDEADYYCAAWDDSLNGVVFGGGTK | | | | | | |
| | | | LTVL | | | | | | |
| | COV2- | 1504 | DIQMTQSPSSLSASVGDRVTITCRASQ | IGHV4-34 | IGHJ6 | IGKV1-39 | IGKJ4 | 2513 | ARVGGYYYYY |
| | 2417 | | SITSYLNWYQQRPGKAPKLLIYAASSLQ | (Human) | (Human) | (Human) | (Human) | | MDV |
| | | | SGVPSRFSGSGSGTDFTLSISSLQPEDF | | | | | | |
| | | | ATYYCQQSYTTLLTFGGGTKVEIK | | | | | | |
| | COV2- | 1505 | QSALTQPASVSGSPGQSITISCTGTSSD | IGHV3-23 | IGHJ6 | IGLV2-14 | IGU3 | 2514 | AKPYGMDV |
| | 2418 | | VGGYNFVSWHQQHPGKAPKNMIYD | (Human) | (Human) | (Human) | (Human) | | |
| | | | VSKRPSGVSNRFSGSKSGNTASLTISGL | | | | | | |
| | | | HTEDEADYYCSSYTSTSTPWVFGGGTK | | | | | | |
| | | | LTVL | | | | | | |
| | COV2- | 1506 | EIVMTQSPGTLSVSPGERATLSCRASQ | IGHV3-30 | IGHJ3 | IGKV3-15 | IGKJ5 | 2515 | ARDRSNLERL |
| | 2420 | | SLSSHLAWYQQKPGQAPRLLIYGVSTR | (Human) | (Human) | (Human) | (Human) | | VMTFGGIIAG |
| | | | ATGIPARFSGSGSGTEFTLAISSLQSED | | | | | | AFDI |
| | | | SAVYYCQQYHVWPPITFGQGTRLEIK | | | | | | |
| | COV2- | 1507 | DIVMTQSPDSLAVSLGERATINCKSSQ | IGHV3-30 | IGHJ4 | IGKV4-1 | IGKJ1 | 2516 | ARVNSGSYYS |
| | 2422 | | SVLYSSNNKNYLAWYQQKPGQPPKLLI | (Human) | (Human) | (Human) | (Human) | | YFDY |
| | | | YWASTRESGVPDRFSGSGSGTDFTLTI | | | | | | |
| | | | SSLQAEDVAVYYCQQYYSTPLTFGQGT | | | | | | |
| | | | KVEIK | | | | | | |
| | COV2- | 1508 | DIQMTQSPSSVSASVGDRVTITCRASQ | IGHV4-4 | IGHJ4 | IGKV1-12 | IGKJ4 | 2517 | ASRWGDYFD |
| | 2427 | | GISSWLAWYQQKPGKAPKLLIYAASSL | (Human) | (Human) | (Human) | (Human) | | SSGAYDS |
| | | | QSGVPSRFSGSGSGTDFTLTITSLQPED | | | | | | |
| | | | FATYYCQQGNSFPLTFGGGTKVEIK | | | | | | |
| | COV2- | 1509 | QSVLTQPPSVSGAPGQRVTISCTGSSS | IGHV3-33 | IGHJ4 | IGLV1-40 | IGU3 | 2518 | ARKGPLWRF |
| | 2428 | | NIGAGYDVHWYQQIPGTAPKLLFYGN | (Human) | (Human) | (Human) | (Human) | | DY |
| | | | NNRPSGVPDRFSDSKSGTSASLAITGL | | | | | | |
| | | | QAEDEADYYCQSYDSSLSAWVFGGGT | | | | | | |
| | | | KLTVL | | | | | | |
| | COV2- | 1510 | DIQMTQSPSTLSASVGDRVTITCRASH | IGHV3-53 | IGHJ4 | IGKV1-5 | IGKJ1 | 2519 | ARESTQ |
| | 2429 | | SISSWLAWYQQKPGKAPKLLIYKASSL | (Human) | (Human) | (Human) | (Human) | | |
| | | | ESGVPSRFSGSGSGTEFTLTISSLQPDD | | | | | | |
| | | | FATYYCQQYNTYSQTFGQGTKVEIK | | | | | | |
| | COV2- | 1511 | EIVLTQSPATLSLSPGERATLSCRASQS | IGHV4-39 | IGHJ6 | IGKV3-11 | IGKJ5 | 2520 | ARAPFQLLDK |
| | 2430 | | VSSYLAWYQQKPGQAPRLLIYDASNR | (Human) | (Human) | (Human) | (Human) | | YYFFYYMDV |
| | | | ATGIPARFSGSGSGTDFTLTISSLEPED | | | | | | |
| | | | FAVYYCQQRSNWPPGVTFGQGTRLEIK | | | | | | |
| | COV2- | 1512 | DIQMTQSPSSLSASVGDRVTITCQASQ | IGHV3-53 | IGHJ4 | IGKV1-33 | IGKJ2 | 2521 | ARHIPAWGY |
| | 2434 | | DIRNYLNWYQQKPGKAPELLIYDASNL | (Human) | (Human) | (Human) | (Human) | | K |
| | | | ETGVPSRFSGSGSGTDFIFTISSLQPED | | | | | | |
| | | | IATYYCHQYDYLPYTFGQGTKLDIK | | | | | | |
| | COV2- | 1513 | DIQMTQSPSSLSASVGDRVTITCRASQ | IGHV3-23 | IGHJ3 | IGKV1-39 | IGKJ2 | 2522 | AKLLGSGITLD |
| | 2438 | | SISSYLNWYQQKPGKAPKLLIYAATSLQ | (Human) | (Human) | (Human) | (Human) | | NDAFDI |
| | | | SGVPSRFSGSGSGTDFTLTISSLQPEDF | | | | | | |
| | | | ATYYCQQTYSTPLYTFGQGTKLEIK | | | | | | |
| | COV2- | 1514 | EIVLTQSPATLSLSPGERATLSCRASQS | IGHV1-69 | IGHJ4 | IGKV3-11 | IGKJ3 | 2523 | ARIGHFDSSG |
| | 2441 | | VSSFLAWYQQKPGQAPRLLIYDASNR | (Human) | (Human) | (Human) | (Human) | | YYLDY |
| | | | PTGIPARFTGSGSGTDFTLTISSLEPED | | | | | | |
| | | | FAVYYCQHRTNWPPLFTFGPGTKVDIK | | | | | | |
| | COV2- | 1515 | SYELTQPPSVSVSPGQTARITCSGDALP | IGHV3-23 | IGHJ4 | IGLV3-25 | IGU2 | 2524 | AKDFGSGIVG |
| | 2444 | | NQYAYWYQQKPGQAPVLVMCKDSE | (Human) | (Human) | (Human) | (Human) | | ATGFDF |
| | | | RPSGIPERFSGSSSGTTVTLTISGVQAE | | | | | | |
| | | | DEADYYCQSADSSGTYVVFGGGTKLT | | | | | | |
| | | | VL | | | | | | |
| | COV2- | 1516 | DIQMTQSPSTLSASVGDRVTITCRASQ | IGHV3-20 | IGHJ6 | IGKV1-5 | IGKJ1 | 2525 | ARRRSSSRYSS |
| | 2445 | | SVSTWLAWYQQKPGKAPNLLIYEASSL | (Human) | (Human) | (Human) | (Human) | | GWYMYYYY |
| | | | ESGVPSRFSGSGSGTEFTLTISSLQPDD | | | | | | MDV |
| | | | FATYYCQQYNTYSQTFGQGTKVEIK | | | | | | |
| | COV2- | 1517 | QPVLTQPPSASASLGASVTLTCTLSSGY | IGHV5-51 | IGHJ4 | IGLV9-49 | IGU2 | 2526 | ATHRCSGGFC |
| | 2446 | | SNYKVDWYQQRPGKGPRFVMRVGT | (Human) | (Human) | (Human) | (Human) | | YLAY |
| | | | GGIVGSKGDGIPDRFSVLGSGLNRYLTI | | | | | | |
| | | | KNIQEEDESDYHCGADHGSGSNFVFV | | | | | | |
| | | | VFGGGTKLTVL | | | | | | |
| | COV2- | 1518 | DIVMTQSPDSLAVSLGERATINCKSSQ | IGHV3-30 | IGHJ1 | IGKV4-1 | IGKJ1 | 2527 | ARASYNSNW |
| | 2449 | | SLLYTSNNKNYLAWYQQKPGQPPKLLI | (Human) | (Human) | (Human) | (Human) | | SIGEYFRD |
| | | | YWASTRESGVPDRFSGSGSGTDFTLTI | | | | | | |
| | | | SSLQAEDVAVYYCQQYYSPPWTFGQG | | | | | | |
| | | | TKVEIK | | | | | | |
| | COV2- | 1519 | DIQMTQSPSSLSASVGDRVTITCQASQ | IGHV3-30 | IGHJ4 | IGKV1-33 | IGKJ5 | 2528 | AKQGGLYCSG |
| | 2450 | | DISNYLHWYQQKPGKAPKLLIYDASNL | (Human) | (Human) | (Human) | (Human) | | TNCWGGYLD |
| | | | ETGVPSRFSGSGSGTDFTFTISSLQPED | | | | | | Y |
| | | | IATYYCQQYHNLPPITFGQGTRLEIK | | | | | | |
| | COV2- | 1520 | SYELTQPPSVSVSPGQTASITCSGDKLG | IGHV2-5 | IGHJ4 | IGLV3-1 | IGU1 | 2529 | VHRHVSGAF |
| | 2451 | | ETYVSWYQQKPGQSPVLVIYEDSKRPS | (Human) | (Human) | (Human) | (Human) | | DY |
| | | | GIPERFSGSNSGNTATLTISGTQAMDE | | | | | | |
| | | | ADYYCQAWDSSTGGVFGTGTKVTVL | | | | | | |
| | COV2- | 1521 | DIQMTQSPSSLSASVGDRVTITCQASL | IGHV7-4- | IGHJ3 | IGKV1-33 | IGKJ5 | 2530 | ARARLLGYCS |
| | 2453 | | DISKYLNWYQHKPGKAPNLLIYDAFNL | 1 | (Human) | (Human) | (Human) | | STSCYTIGWG |
| | | | ERGVPSRFSGSGSGTDFTFTISSLQPED | (Human) | | | | | AFDI |
| | | | IATYYCQQYDNLPPGVSTTFGQGTRLE | | | | | | |
| | | | IK | | | | | | |
| | COV2- | 1522 | SYELTQPPSVSVSPGQTARITCSGDALP | IGHV3-33 | IGHJ4 | IGLV3-10 | IGU3 | 2531 | AREGQGTYLD |
| | 2454 | | KKYAHWYQQKSGQAPVLVIYEDSKRP | (Human) | (Human) | (Human) | (Human) | | Y |
| | | | SGIPERFSGSSSGTMATLTISGAQVED | | | | | | |
| | | | EADYYCCSTDSSGNQRVFGGGTKLTVL | | | | | | |
| | COV2- | 1523 | QSALTQPASVSGSPGQSITISCTGTSSD | IGHV3-48 | IGHJ4 | IGLV2-14 | IGU2 | 2532 | AREARSRYFD |
| | 2455 | | IGGYNYVSWYQQHPGKAPKLLIYDVS | (Human) | (Human) | (Human) | (Human) | | WLPSYYFDY |
| | | | NRPSGVSTRFSGSKSGNTASLTISGLQ | | | | | | |
| | | | AEDEADYYCSSYTSSSTHVVFGGGTKL | | | | | | |
| | | | TVL | | | | | | |
| | COV2- | 1524 | SYELTQPPSVSVSPGQTARITCSGDALP | IGHV3-30 | IGHJ4 | IGLV3-25 | IGU2 | 2533 | ARDPSPLVLIT |
| | 2458 | | RQYTYWYQQKPGQAPVLVIYKDSERP | (Human) | (Human) | (Human) | (Human) | | SIDY |
| | | | SGIPERFSGSSSGTTVTLTISGVQAEDE | | | | | | |
| | | | ADYYCQSADTIGTYWVFGGGTKLTVL | | | | | | |
| | COV2- | 1525 | EIVMTQSPATVSVSPGERATLSCRASQ | IGHV4-39 | IGHJ5 | IGKV3-15 | IGKJ2 | 2534 | ARRGNYYDSK |
| | 2459 | | SVSSNLAWYQQKPGQAPRLLIYGAST | (Human) | (Human) | (Human) | (Human) | | NWFDP |
| | | | RATGIPARFSASGSGTEFTLTISSLQSE | | | | | | |
| | | | DFAVYYCQQYNNWPPMYTFGQGTKVE | | | | | | |
| | | | IK | | | | | | |
| | COV2- | 1526 | DIQMTQSPSSLSASVGDRVTITCQASQ | IGHV4-31 | IGHJ4 | IGKV1-33 | IGKJ2 | 2535 | ARGGSGSYSL |
| | 2461 | | DITNYLNWYQQKPGKAPKLLIYDASNL | (Human) | (Human) | (Human) | (Human) | | FDY |
| | | | ETGVPSRFSGSGSGTDFTFTISSLQPED | | | | | | |
| | | | FATYYCQQYDNLYSVHFGQGTKLEIK | | | | | | |
| | COV2- | 1527 | DIQMTQSPSSLSASVGDRVTITCRASQ | IGHV3-13 | IGHJ5 | IGKV1-39 | IGKJ1 | 2536 | ARVLYDSSGF |
| | 2462 | | SISSYLNWYQQKPGKAPKLLIYAASSLK | (Human) | (Human) | (Human) | (Human) | | YNWFDP |
| | | | SGVPSRFGGSGSGTDFTLTISSLQPEDF | | | | | | |
| | | | ATYYCQQSYEMPPWTFGQGTKVEIE | | | | | | |
| | COV2- | 1528 | SYELTQPPSVSVSPGQTARITCSGDALP | IGHV4-31 | IGHJ2 | IGLV3-25 | IGU1 | 2537 | ARDLGDGYN |
| | 2464 | | NQYAYWYHQKPGQAPVVVIYKDSER | (Human) | (Human) | (Human) | (Human) | | LRVPAYFDL |
| | | | PSGIPQRFSGSSSGTTVTLTISGVQAED | | | | | | |
| | | | EADYYCQSADSSGYVFGTGTKVTVL | | | | | | |
| | COV2- | 1529 | QSALTQPASVSGSPGQSITISCTGTSSD | IGHV4-4 | IGHJ4 | IGLV2-14 | IGU1 | 2538 | ARVDHVNVR |
| | 2465 | | VGGYNYVSWYQQHPGKAPKLMIYDV | (Human) | (Human) | (Human) | (Human) | | DY |
| | | | SKRPSGVSNRFSGSKSGNTASLTISGLQ | | | | | | |
| | | | AEDEADYYCSSYTSSSIPYVFGTGTKVT | | | | | | |
| | | | VL | | | | | | |
| | COV2- | 1530 | EIVLTQSPAILSLSPGERATLSCRASQS | IGHV3-21 | IGHJ6 | IGKV3-11 | IGKJ1 | 2539 | ARVNGNSN |
| | 2466 | | VSSYLAWYQQKPGQAPRLLIYDTSNRAT | (Human) | (Human) | (Human) | (Human) | | WNFGSYYYYY |
| | | | GIPARFSGSGSGTDFTLTISSLEPEDFA | | | | | | MDV |
| | | | FYYCQQRGNWWTFAQGTKVEIK | | | | | | |
| | COV2- | 1531 | SYELTQPPSVSVSPRQTARITCSGDALP | IGHV3-33 | IGHJ4 | IGLV3-10 | IGU3 | 2540 | AREGQMAAT |
| | 2473 | | KKYAYWYQQKSGQAPVLVIYEDSKRP | (Human) | (Human) | (Human) | (Human) | | TGIDY |
| | | | SGIPERFSGSSSGTMATLTISGAQVED | | | | | | |
| | | | EADYYCYSTDTSGNHWVFGGGTKLTV | | | | | | |
| | | | L | | | | | | |
| | COV2- | 1532 | SYELTQPPSVSVSPGQTARITCSGDALP | IGHV3-15 | IGHJ3 | IGLV3-10 | IGU3 | 2541 | TTLTYYYDSSA |
| | 2474 | | KKYAYWYQQKSGQAPVLVISEDSKRP | (Human) | (Human) | (Human) | (Human) | | YLNDAFDI |
| | | | SGIPERFSGSSSGTVATLTISGAQVEDE | | | | | | |
| | | | ADYYCYSTDSSGNHRVFGGGTKLTVL | | | | | | |
| | COV2- | 1533 | EIVLTQSPGTLSLSPGERATLSCRASQS | IGHV3-9 | IGHJ4 | IGKV3-20 | IGKJ2 | 2542 | AKGIYYDIFM |
| | 2478 | | VSSTFLAWYQQKPGQAPRLLIFGASSR | (Human) | (Human) | (Human) | (Human) | | PLLD |
| | | | ATGIPDRFSGSGSGTDFTLTISRLEPED | | | | | | |
| | | | FAVYYCHQYGTSPYTFGQGTKLEIK | | | | | | |
| | COV2- | 1534 | EIVMTQSPATLSVSPGERVTLSCRASQ | IGHV1-69 | IGHJ5 | IGKV3-15 | IGKJ4 | 2543 | ARGVVAATP |
| | 2479 | | SVSSNLAWYQQKPGQAPRLLIYGAST | (Human) | (Human) | (Human) | (Human) | | GWFDP |
| | | | RATGIPARFSGGGSGTEFTLTISSLQSE | | | | | | |
| | | | DFAVYYCQQYNNFLTFGGGTKVEIK | | | | | | |
| | COV2- | 1535 | DIVMTQSPDSLAVSLGERATINCKSSQ | IGHV1-69 | IGHJ5 | IGKV4-1 | IGKJ2 | 2544 | ARGWFGELL |
| | 2481 | | SVLYSSNNKNYLAWYQQKPGQPPKLLI | (Human) | (Human) | (Human) | (Human) | | KGTYWFDP |
| | | | YWASTRESGVPDRFSGSGSGTDFTLTI | | | | | | |
| | | | SSLQAEDVAVYYCQQYYSTPGFGQGT | | | | | | |
| | | | KLEIK | | | | | | |
| | COV2- | 1536 | QSVLTQPPSVSGAPGQRVTLSCTGSNS | IGHV1-69 | IGHJ2 | IGLV1-40 | IGU3 | 2545 | ARVGVSGFKS |
| | 2485 | | NIGAGYDVHWYQQLPGTAPKLLIYGN | (Human) | (Human) | (Human) | (Human) | | GSNWYFDL |
| | | | SNRPSGVPDRFSGSKSGTSASLAITGL | | | | | | |
| | | | QAEDEADYYCQSYDSSLSDSVFGGGT | | | | | | |
| | | | KVTVL | | | | | | |
| | COV2- | 1537 | SYELTQPPSVSVSPGQTARITCSGDALP | IGHV3-74 | IGHJ4 | IGLV3-25 | IGU3 | 2546 | AREVEQLAH |
| | 2488 | | NQYAYWYQQKPGQAPVLVIYKDSERP | (Human) | (Human) | (Human) | (Human) | | MVDY |
| | | | SGIPERFSGSSSGTTVTLTISGVQAEDE | | | | | | |
| | | | ADYYCQSADSSGTSWVFGGGTKLTVL | | | | | | |
| | COV2- | 1538 | EIVLTQSPGTLSLSPGERASLSCRASQS | IGHV4-39 | IGHJ2 | IGKV3-20 | IGKJ4 | 2547 | ARQWKWFG |
| | 2489 | | VSSSYLAWYQQKPGQAPRLLIYGASSR | (Human) | (Human) | (Human) | (Human) | | EAWYFDL |
| | | | ATGIPDRFSGSGSGTDFTLTISRLEPED | | | | | | |
| | | | FAVYYCQQYGSSPFTFGGGTKVEIK | | | | | | |
| | COV2- | 1539 | DIQMTQSPSTLSASVGDRVTITCRASQ | IGHV3-7 | IGHJ4 | IGKV1-5 | IGKJ4 | 2548 | ARDPYDLYGD |
| | 2490 | | SISSWLAWYQQKPRKAPKLLIYKASTLE | (Human) | (Human) | (Human) | (Human) | | YGGTFDY |
| | | | SGVPSRFSGSGSGTEFTLTISSLQPDDF | | | | | | |
| | | | ATYYCQQYNSYSLTFGGGTKVEIK | | | | | | |
| | COV2- | 1540 | EIVLTQSPATLSVSPGERATLSCRASQS | IGHV1-69 | IGHJ6 | IGKV3-15 | IGKJ1 | 2549 | AREDYYGSGS |
| | 2495 | | VSSNLAWYQQKPGQAPRLLIYGASTR | (Human) | (Human) | (Human) | (Human) | | LVDPYYYYRM |
| | | | ATGIPARFSGSGSGTDFTLTISSLQSED | | | | | | DV |
| | | | FALYYCQQYNNWWRTFGQGTKVEIN | | | | | | |
| | COV2- | 1541 | SYELTQPPSVSVSPGQTARITCSGDALP | IGHV3-33 | IGHJ4 | IGLV3-10 | IGU3 | 2550 | VRDLALFEVVI |
| | 2496 | | KKYAYWYQQKSGQAPVLVIYEDSKRP | (Human) | (Human) | (Human) | (Human) | | QQGV |
| | | | SGIPERFSGSSSGTMATLTISGAQVED | | | | | | |
| | | | EADYYCYSTDSSGNHWVFGGGTKLTV | | | | | | |
| | | | L | | | | | | |
| | COV2- | 1542 | SSELTQDPAVSVALGQTVRITCQGDSL | IGHV4-39 | IGHJ3 | IGLV3-19 | IGU2 | 2551 | ARHTVDCGG |
| | 2499 | | RSYYASWYQQKPGQAPLLVIYGKNNR | (Human) | (Human) | (Human) | (Human) | | DCFPNDAFDI |
| | | | PSGIPDRFSGSSSGNTPSLTITGAQAED | | | | | | |
| | | | EADYYCNFRDSSGHHPVFGEGTKLTVL | | | | | | |
| | COV2- | 1543 | QSVLTQPPSASGTPGQRVIISCSGSSSN | IGHV1-2 | IGHJ4 | IGLV1-44 | IGU2 | 2552 | ARVVVLGYGR |
| | 2504 | | IGSNTVKWYHQLPGTAPKLLICSNNQR | (Human) | (Human) | (Human) | (Human) | | PNNYYDGRN |
| | | | PSGVPDRFSGSKSDTSASLAISGLQSED | | | | | | VWDY |
| | | | EADYYCAAWDDSLNALVFGGGTKLTV | | | | | | |
| | | | L | | | | | | |
| | COV2- | 1544 | EIVMTQSPATLSVSPGERATLSCRASQ | IGHV3- | IGHJ4 | IGKV3-15 | IGKJ1 | 2553 | ARPRGGSYQT |
| | 2509 | | SVSSNLAWYQQKPGQAPRLLIYGAST | 303 | (Human) | (Human) | (Human) | | CFDY |
| | | | RATGIPARFSGSGSGTEFTLTISSLQSE | (Human) | | | | | |
| | | | DFAVYYCQQYNNWPGTFGQGTKVEI | | | | | | |
| | | | K | | | | | | |
| | COV2- | 1545 | SYELTQPSSVSVSPGQTARITCSGDVLA | IGHV7-4- | IGHJ4 | IGLV3-27 | IGU2 | 2554 | ARWGPDYGD |
| | 2510 | | KNYARWFQQKPGQAPVLVIYKDSERP | 1 | (Human) | (Human) | (Human) | | YASNDY |
| | | | SGIPKRFSGSSSGTTVTLTISGAQVEDE | (Human) | | | | | |
| | | | ADYYCYSAADNNRVFGGGTKLTVL | | | | | | |
| | COV2- | 1546 | SSELTQDPAVSVALGQTVRITCQGDSL | IGHV3-20 | IGHJ3 | IGLV3-19 | IGU2 | 2555 | AVIMSPIPRYS |
| | 2514 | | RSYYASWYQQKPGQVPILVIYDKNNR | (Human) | (Human) | (Human) | (Human) | | GYDWAGDAF |
| | | | PSGIPDRFSGSSSGNTASLTITGAQAED | | | | | | DI |
| | | | EADYYCNSRDSSGNAVVFGGGTKLTV | | | | | | |
| | | | L | | | | | | |
| | COV2- | 1547 | DIQMTQSPSSLSASVGDRVTITCQASQ | IGHV7-4- | IGHJ5 | IGKV1-33 | IGKJ3 | 2556 | ARGLVGRIDP |
| | 2515 | | DISNSLSWYQQKPGKAPKLLIYDASNL | 1 | (Human) | (Human) | (Human) | | |
| | | | KTGVPSRFSGSGSGTDFTFTISSLQPED | (Human) | | | | | |
| | | | IATYYCQQYDNLLLFTFGPGTKVDIK | | | | | | |
| | COV2- | 1548 | EIVLTQSPATLSLSPGERATLSCRASQS | IGHV1-46 | IGHJ3 | IGKV3-11 | IGKJ1 | 2557 | ARDQQIVPH |
| | 2516 | | VSSYLAWYQQKPGQAPRLLIYDASNR | (Human) | (Human) | (Human) | (Human) | | ADGFDI |
| | | | ATGIPARFSGSGSGTDFTLTISSLEPED | | | | | | |
| | | | FAVYYCQQRSNWPPRTWTFGQGTKVE | | | | | | |
| | | | IK | | | | | | |
| | COV2- | 1549 | EIVLTQSPATLSLSPGERATLSCRASQS | IGHV1-69 | IGHJ1 | IGKV3-11 | IGKJ4 | 2558 | ARIGSYPEYFQ |
| | 2517 | | VSSFLAWYQQKPGQAPRLLIYDASNR | (Human) | (Human) | (Human) | (Human) | | |
| | | | ATGIPARFSGSGSGTDFTLTISSLEPED | | | | | | |
| | | | FAVYYCHYRSNWPPVLTFGGGTKVEIE | | | | | | |
| | COV2- | 1550 | SSELTQDPAVSVALGQTVRITCQGDSL | IGHV3-9 | IGHJ6 | IGLV3-19 | IGU2 | 2559 | AKDIIRQGED |
| | 2518 | | RSYYASWYQQKPGQVPILVIYDKNNR | (Human) | (Human) | (Human) | (Human) | | GMDV |
| | | | PSGIPDRFSGSSSGNTASLTITGAQAED | | | | | | |
| | | | EADYYCNSRDSSGNAVVFGGGTKLTV | | | | | | |
| | COV2- | 1551 | SSELTQDPAVSVALGQTVRITCQGDSL | IGHV3-7 | IGHJ6 | IGLV3-19 | IGU3 | 2560 | ARDVGGYSG |
| | 2520 | | RSYYANWYQQKPGQAPVVVIFGKNN | (Human) | (Human) | (Human) | (Human) | | YDLGFDYYYY |
| | | | RPSGIPDRFSGSNSGNTASLTITGAQA | | | | | | MDV |
| | | | EDEADYYCNSRDSSGYIWGWMFGGG | | | | | | |
| | | | TKLTVL | | | | | | |
| | COV2- | 1552 | SYVLTQPPSVSVAPGKTARITCGGNNI | IGHV3-30 | IGHJ3 | IGLV3-21 | IGU3 | 2561 | AKDRTAVFLF |
| | 2521 | | GSKSVHWYQQKPGQAPVVVVYDDSD | (Human) | (Human) | (Human) | (Human) | | FGLGDAFDI |
| | | | RPSGLPERFSGSNSGNTATLTISRVEA | | | | | | |
| | | | GDEADYYCQVWDSSSDHWVFGGGT | | | | | | |
| | | | KLTVL | | | | | | |
| | COV2- | 1553 | QSVLTQPPSVSGAPGQRVTISCTGSSS | IGHV5-51 | IGHJ4 | IGLV1-40 | IGU3 | 2562 | ASALRERGVQ |
| | 2524 | | NIGAGYDVHWYQQLPGTAPKLLIFINS | (Human) | (Human) | (Human) | (Human) | | LWSV |
| | | | NRPSGVPDRFSGSKSGTSASLAITGLQ | | | | | | |
| | | | AEDEADYYCQSYDSSLGALFGGGTKLT | | | | | | |
| | | | VL | | | | | | |
| | COV2- | 1554 | QLVLTQSPSASASLGASVKLTCTLSSGH | IGHV3-23 | IGHJ4 | IGLV4-69 | IGU3 | 2563 | AKDPASGIVG |
| | 2525 | | TTYAIAWHQQQPEKGPRYLMKLNSD | (Human) | (Human) | (Human) | (Human) | | PTHFDY |
| | | | GSHTRGDGIPDRFSGSSSGAERYLTISS | | | | | | |
| | | | LQSEDEADYYCQSWDTGIGVFGGGTK | | | | | | |
| | | | LTVL | | | | | | |
| | COV2- | 1555 | SYELTQPPSVSVSPGQTASITCFGDKLG | IGHV1-24 | IGHJ4 | IGLV3-1 | IGU2 | 2564 | ATGFAVFGRA |
| | 2526 | | DKYACWFQQKPGQSPVLIIYQGAKRP | (Human) | (Human) | (Human) | (Human) | | AVPY |
| | | | SGIPERFSGSNSGNTATLTISGTQAMD | | | | | | |
| | | | EADYYCQAWDSSTVVFGGGTKLTVL | | | | | | |
| | COV2- | 1556 | QSALTQPASVSGSPGQSITISCTGTSSD | IGHV4-30 | IGHJ4 | IGLV2-23 | IGU2 | 2565 | ARFRRSYGSG |
| | 2527 | | VGSYNLVSWYQQHPGKAPKLMIYEVS | (Human) | (Human) | (Human) | (Human) | | SYYNISFDY |
| | | | KRPSGVSNRFSGSKSGNTASLTISGLQ | | | | | | |
| | | | AEDEADYYCCSYVGSSTYVVFGGGTKL | | | | | | |
| | | | TVL | | | | | | |
| | COV2- | 1557 | SYELTQPPSVSVSPGQTARITCSADALS | IGHV7-4- | IGHJ4 | IGLV3-25 | IGU3 | 2566 | VREYGSGHPL |
| | 2529 | | KQYAYWYQQKPGQAPVVVIYKDSERP | 1 | (Human) | (Human) | (Human) | | PI |
| | | | SGIPERFSGSSSGTTVTLTISGVQAEDE | (Human) | | | | | |
| | | | ADYYCQSADSSSHWVFGGGTKLTVL | | | | | | |
| | COV2- | 1558 | NFMLTQPHSVSESPGKTVTISCTGNSG | IGHV4-59 | IGHJ3 | IGLV6-57 | IGU2 | 2567 | ARATWLRDA |
| | 2531 | | SIASNYVQWYQQRPGSAPTTVIYEDS | (Human) | (Human) | (Human) | (Human) | | FGI |
| | | | QRPSGVPDRFSGSIDSSSNSASLTISGL | | | | | | |
| | | | KTEDEADYYCQSFDSGNVVFGGGTKL | | | | | | |
| | | | TVL | | | | | | |
| | COV2- | 1559 | QSVLTQPPSASGTPGQRVTISCSGSSS | IGHV1-46 | IGHJ5 | IGLV1-44 | IGU2 | 2568 | ARGFHVPAAL |
| | 2532 | | NIGSNTVNWYQQLPGTAPKLLIYNNN | (Human) | (Human) | (Human) | (Human) | | RNWFDP |
| | | | QRPSGVPDRLSGSKSGTSASLAISGLQ | | | | | | |
| | | | SEDEADYYCVAWDDSRNGLVFGGGT | | | | | | |
| | | | KLTVL | | | | | | |
| | COV2- | 1560 | DIVMTQSPDSLAVSLGERATINCKSSQ | IGHV4-31 | IGHJ4 | IGKV4-1 | IGKJ2 | 2569 | AREANDSGSF |
| | 2533 | | SVLYSSNNKNYLAWYQQKPGQPPKLLI | (Human) | (Human) | (Human) | (Human) | | YNGPFDY |
| | | | YWASTRESGVPDRFSGSGSGTDFTLTI | | | | | | |
| | | | SSLQAEDVAVYYCQQYYSTRTFGQGT | | | | | | |
| | | | KLEIK | | | | | | |
| | COV2- | 1561 | DIVMTQSPDSLAVSLGERATINCKSSQ | IGHV4-61 | IGHJ6 | IGKV4-1 | IGKJ2 | 2570 | ARGGLLWFG |
| | 2536 | | SVLYSSNNKNYLAWYQQKPGQPPKLLI | (Human) | (Human) | (Human) | (Human) | | GAGNYMDV |
| | | | YWASTRESGVPDRFSGSGSGTDFTLTI | | | | | | |
| | | | SSLQAEDVAVYYCQQYYSTPPYTFGQG | | | | | | |
| | | | TKVEIK | | | | | | |
| | COV2- | 1562 | QSVLTQPPSASGTPGQRVTISCSGSNS | IGHV1-8 | IGHJ4 | IGLV1-44 | IGU3 | 2571 | ARMRTGWPT |
| | 2539 | | NIGSYTVNWYQQFPGTAPKLLIYDNN | (Human) | (Human) | (Human) | (Human) | | HGRPDDF |
| | | | QRTSGVPDRFSGSKSGTSASLAISGLQ | | | | | | |
| | | | SEDEASYYCLAWDDSLNGLVFGGGTK | | | | | | |
| | | | LTVL | | | | | | |
| | COV2- | 1563 | QSVLTQPPSASGTPGQRVTISCSGSSS | IGHV4-34 | IGHJ5 | IGLV1-47 | IGU3 | 2572 | ARGPPVTTFF |
| | 2545 | | NIGSNYVYWYQQLPGTAPKLLIYSSNQ | (Human) | (Human) | (Human) | (Human) | | VFSLLFDP |
| | | | RPSGVPDRFSGSKSGTSASLAISGLRSE | | | | | | |
| | | | DEADYYCAAWDASLSGWVFGGGTKL | | | | | | |
| | | | TVL | | | | | | |
| | COV2- | 1564 | SYELTQPPSVSVSPGQTARITCSADALP | IGHV3-30 | IGHJ3 | IGLV3-25 | IGU3 | 2573 | AKEGEWELR |
| | 2546 | | KHYAYWYQQRPGQAPVLVIYKDIERP | (Human) | (Human) | (Human) | (Human) | | GNALDI |
| | | | SGIPERFSGSSSGTTVTLTITGAQAEDE | | | | | | |
| | | | ADYYCQSVDGSGSSVVFGGGTKLAVL | | | | | | |
| | COV2- | 1565 | SYELTQPPSVSVSPGQTASITCSGDKLG | IGHV3-33 | IGHJ3 | IGLV3-1 | IGU2 | 2574 | AREGQWPN |
| | 2549 | | DKYACWYQQKPGQSPVLVIHQDSQR | (Human) | (Human) | (Human) | (Human) | | QAFDI |
| | | | PSGIPERFSGSNSGNTATLTISGTQAM | | | | | | |
| | | | DEADYYCQAWDSSTHVVFGGGTKLT | | | | | | |
| | | | VL | | | | | | |
| | COV2- | 1566 | QSALTQPASVSGSPGQSITISCTGTSSD | IGHV4-39 | IGHJ4 | IGLV2-14 | IGU1 | 2575 | ASGPPYMATF |
| | 2551 | | VGGYNYVSWFQHHPDKAPRLMIYDV | (Human) | (Human) | (Human) | (Human) | | SYYFDY |
| | | | SKRPSGVSNRFSGSKSGNTASLTISGLQ | | | | | | |
| | | | AEDEADYYCSSYTSSSTPFVFGTGTKVT | | | | | | |
| | | | VL | | | | | | |
| | COV2- | 1567 | QPVLTQSSSASASLGSSVKLTCTLSSGH | IGHV3-11 | IGHJ4 | IGLV4-60 | IGU3 | 2576 | ARDPIRDGV |
| | 2552 | | SSYIIAWHQQQPGKAPRYLMKLEGSG | (Human) | (Human) | (Human) | (Human) | | WGLNENDY |
| | | | SYNKGSGVPDRFSGSSSGADCYLTISN | | | | | | |
| | | | LQSEDEADYYCETWDSNTRVFAGGTK | | | | | | |
| | | | LTVL | | | | | | |
| | COV2- | 1568 | DIQMTQSPSSLSASVGDRVTITCRASQ | IGHV3-30 | IGHJ4 | IGKV1-39 | IGKJ2 | 2577 | ARSPPASYYN |
| | 2553 | | SISSYLNWYQQKPGKAPKLLIYAASSLQ | (Human) | (Human) | (Human) | (Human) | | PSTGYFDY |
| | | | SGVPSRFSGSGSGTDFTLTISSLQPEDF | | | | | | |
| | | | ATFYCQQSYSTPMHTFGQGTKLEIK | | | | | | |
| | COV2- | 1569 | DIQLTQSPSFLSASVGDRVTITCRASQG | IGHV1-46 | IGHJ4 | IGKV1-9 | IGKJ1 | 2578 | ARDVFWVPA |
| | 2554 | | ISSYLAWYQQKPGKAPKLLIYAASTLQS | (Human) | (Human) | (Human) | (Human) | | ASSFDY |
| | | | GVPSRFSGSGSGTEFTLTISSLQPEDFA | | | | | | |
| | | | TYYCQQLNSYLGTFGQGTKVEIK | | | | | | |
| | COV2- | 1570 | DIQMTQSPSSVSASVGDRVTITCRASQ | IGHV3- | IGHJ4 | IGKV1-12 | IGKJ3 | 2579 | ARDQEWFRE |
| | 2558 | | GISSWLAWYQQKPGKAPKLLIYDASSL | 303 | (Human) | (Human) | (Human) | | LFLFDY |
| | | | QSGVPSRFSGSGSGTDFTLTISSLQPED | (Human) | | | | | |
| | | | FATYYCQQANSFPPTFGPGTKVDIK | | | | | | |
| | COV2- | 1571 | QSVLTQPPSASGTPGQRVTISCSGSNS | IGHV1-8 | IGHJ4 | IGLV1-44 | IGU2 | 2580 | ARMRTGWPT |
| | 2562 | | NIGSYTVNWYQQFPGTAPKLLIYDNN | (Human) | (Human) | (Human) | (Human) | | HGRPDDF |
| | | | QRTSGVPDRFSGSKSGTSASLAISGLQ | | | | | | |
| | | | SEDEANYYCLVWDDSLNGLVFGGGTK | | | | | | |
| | | | LTVL | | | | | | |
| | COV2- | 1572 | QSALTQPASVSGSPGQSITISCTGTSSD | IGHV4-34 | IGHJ4 | IGLV2-23 | IGU1 | 2581 | ARGWGWGA |
| | 2563 | | VGSYNLVSWYQQHPGKAPKLMIYEVS | (Human) | (Human) | (Human) | (Human) | | VAGRAEYYFD |
| | | | KRPSGVSNRFSGSKSGNTASLTISGLQ | | | | | | Y |
| | | | AEDEADYYCCSYAGSSTWGFGTGTKV | | | | | | |
| | | | TVL | | | | | | |
| | COV2- | 1573 | SYVLTQPPSVSVAPGKTARITCGGNNI | IGHV3- | IGHJ6 | IGLV3-21 | IGU2 | 2582 | ARAQGGNYY |
| | 2564 | | GTKGVHWYQQKPGQAPVLVVYDDS | 303 | (Human) | (Human) | (Human) | | YGMDV |
| | | | DRPSGIPGRFSGSNSGNTATLTISRVEA | (Human) | | | | | |
| | | | GDEADYFCQVWDSSSDHHVVFGGGT | | | | | | |
| | | | KLTVL | | | | | | |
| | COV2- | 1574 | DIVMTQSPLSLPVTPGEPASISCRSSQS | IGHV4-61 | IGHJ4 | IGKV2-28 | IGKJ4 | 2583 | ARGAASFDY |
| | 2565 | | LLHSDGYTYLDWYLQKPGQSPQLLIYL | (Human) | (Human) | (Human) | (Human) | | |
| | | | GSKRASGVPDRFSGSGSGTDFTLKISR | | | | | | |
| | | | VEAEDVGVYYCMQALQTPLTFGGGTK | | | | | | |
| | | | VEIK | | | | | | |
| | COV2- | 1575 | EIVMTQSPGTLSVSPGERATLSCRASQ | IGHV4-39 | IGHJ5 | IGKV3-15 | IGKJ2 | 2584 | ARDPRVVVTA |
| | 2570 | | SVSSNLAWYQQKPGQAPRLLIYGAST | (Human) | (Human) | (Human) | (Human) | | RMYNWFDP |
| | | | RATGVPARFSGSGSGTEFTLTISSLQSE | | | | | | |
| | | | DFAVYYCQQYNNWPPMYTFGQGTKL | | | | | | |
| | | | EIK | | | | | | |
| | COV2- | 1576 | EIVMTQSPATLSVSPGERATLSCRASQ | IGHV4-59 | IGHJ5 | IGKV3-15 | IGKJ1 | 2585 | ARDQRQFQL |
| | 2571 | | SVSSNLAWYQQKPGQAPRLLIYGAST | (Human) | (Human) | (Human) | (Human) | | LGRFGWFDP |
| | | | RATGIPARFSGSGSGTEFTLTISSLQSE | | | | | | |
| | | | DFAVYYCQQYNNWPRTFGQGTKVEIK | | | | | | |
| | COV2- | 1577 | QPVVTQSPSASASLGASVKLTCTLDSG | IGHV4-39 | IGHJ2 | IGLV4-69 | IGU2 | 2586 | ARQWKWFG |
| | 2574 | | HRSYAIAWHQQRPEKGPRFLMRITTD | (Human) | (Human) | (Human) | (Human) | | EAWYFDL |
| | | | GRHTKGDGIPDRFSGSGSGTERYLTISS | | | | | | |
| | | | LQSEDEADYYCQTWGTFGGGTRLTVL | | | | | | |
| | COV2- | 1578 | DIVMTQTPLSLSVTPGQPASISCKSSQS | IGHV7-4- | IGHJ6 | IGKV2D-29 | IGKJ4 | 2587 | ARDQDSGYP |
| | 2582 | | LLHSDGKTYLYWYLQKPGQSPQLLIYE | 1 | (Human) | (Human) | (Human) | | TYYYYYMDV |
| | | | VSNRFSGVPDRFSGSGSGTDFTLKISR | (Human) | | | | | |
| | | | VEAEDVGVYYCMQSIQPPLTFGGGTK | | | | | | |
| | | | VEIK | | | | | | |
| | COV2- | 1579 | DIVMTQSPDSLAVSLGERATINCKSSQ | IGHV1-8 | IGHJ6 | IGKV4-1 | IGKJ4 | 2588 | ARGGIYYLVR |
| | 2583 | | SVLYSSNNKNYLAWYQQKPGQPPKLLI | (Human) | (Human) | (Human) | (Human) | | GFIIGYYGMD |
| | | | YWASTRESGVPDRFSGSGSGTDFTLTI | | | | | | V |
| | | | SSLQAEDVAVYYCQQYYSTPLTFGGGT | | | | | | |
| | | | KVEIK | | | | | | |
| | COV2- | 1580 | QSALTQPASVSGSPGQLITIFCNGSISD | IGHV3-15 | IGHJ4 | IGLV2-14 | IGU2 | 2589 | TTGGYSSYAA |
| | 2584 | | VGGWNYVSWYQQHPDKAPKMMIY | (Human) | (Human) | (Human) | (Human) | | SDY |
| | | | DVRHRPSGVSSRFSGSKSGNTASLTIS | | | | | | |
| | | | GLQAEDEGDYYCSSFTSRGALVLFGG | | | | | | |
| | | | GTKLTVL | | | | | | |
| | COV2- | 1581 | QSALTQPRSVSGSPGQSVTISCTGTSS | IGHV3-48 | IGHJ2 | IGLV2-11 | IGU2 | 2590 | ARVDYYGSGS |
| | 2585 | | DVGGYNYVSWYQQHPGKAPKLMIYD | (Human) | (Human) | (Human) | (Human) | | VYWYFDL |
| | | | VSKRPSGVPDRFSGSKSGNTASLTISGL | | | | | | |
| | | | QAEDEADYYCCSYAGIWVFGGGTKLT | | | | | | |
| | | | VL | | | | | | |
| | COV2- | 1582 | EIVLTQSPGTLSLSPGERATLSCRASQS | IGHV3-9 | IGHJ3 | IGKV3-20 | IGKJ2 | 2591 | AKVGWELSID |
| | 2586 | | VSSSYLGWYHQKPGQAPRLLIYGASSR | (Human) | (Human) | (Human) | (Human) | | AFDL |
| | | | ATGIPDRFSGSGSGTDFTLTISRLEPED | | | | | | |
| | | | FAVYYCQHYGSSRSTFGQGTKLEIK | | | | | | |
| | COV2- | 1583 | DIQMTQSPSSLSASVGDRVTITCRARQ | IGHV2-5 | IGHJ3 | IGKV1-39 | IGKJ4 | 2592 | AHRLWFRDA |
| | 2587 | | SISNYLNWYQQKPGKAPKLLIYAASSL | (Human) | (Human) | (Human) | (Human) | | FDI |
| | | | HSGVPSRFSGSGSGTDFTLTISSLQPED | | | | | | |
| | | | FATYYCQQSYSTPTFGGGTKVEIK | | | | | | |
| | COV2- | 1584 | DIQMTQSPSSLSASVGDRVTITCRASQ | IGHV2-70 | IGHJ6 | IGKV1-39 | IGKJ2 | 2593 | ARIQYQLNG |
| | 2589 | | SISSYLNWYQQKPGKAPKLLIYAASSLH | (Human) | (Human) | (Human) | (Human) | | MDV |
| | | | SGVPSRFSGSGSGTDFTLTISSLQPEDF | | | | | | |
| | | | ATYYCQQSYSTPYTFGQGTKLEIK | | | | | | |
| | COV2- | 1585 | SSELTQDPAVSVALGQTVRITCQGDSL | IGHV1-8 | IGHJ6 | IGLV3-19 | IGU2 | 2594 | ARGRVGYVG |
| | 2590 | | RSYYASWYQQKPRQAPVLIISGNNNR | (Human) | (Human) | (Human) | (Human) | | SGSRGYYYYY |
| | | | PSGIPDRFSGSSSGNTASLTITGAQAED | | | | | | DMDV |
| | | | EADYYCNSRDSSGNHLRVFGGGTKLT | | | | | | |
| | | | VL | | | | | | |
| | COV2- | 1586 | EIVLTQSPGTLSLSPGERATLSCRASQS | IGHV5-51 | IGHJ2 | IGKV3-20 | IGKJ5 | 2595 | ARPDYSSGW |
| | 2602 | | VSSNFLAWYQQKPGQAPRLLIYGASS | (Human) | (Human) | (Human) | (Human) | | FSYWYFDL |
| | | | RATGIPDRFSGSGSGTDFTLTISRLEPE | | | | | | |
| | | | DFAVYYCQQYGRSPITFGQGTRLEIK | | | | | | |
| | COV2- | 1587 | DIQMTQSPSSLSASVGDRVTITCQASQ | IGHV7-4- | IGHJ4 | IGKV1-33 | IGKJ3 | 2596 | ARGRSYGLSL |
| | 2610 | | DISNYLNWYQQKPGKAPKLLIYAASNL | 1 | (Human) | (Human) | (Human) | | GY |
| | | | ETGVPSRFSGSGSGTDFTFTISSLQPED | (Human) | | | | | |
| | | | IATYYCQQYDNLLQFTFGPGTKVDIK | | | | | | |
| | COV2- | 1588 | SYELTQPPSVSVSPGQTARITCSGDALP | IGHV3-33 | IGHJ4 | IGLV3-10 | IGU1 | 2597 | ARESADISSRL |
| | 2611 | | TKYAYWYQQKSGQAPVLVIYDDSKRP | (Human) | (Human) | (Human) | (Human) | | DY |
| | | | SGIPERFSGSSSGTMATLTISGAQVED | | | | | | |
| | | | EADYYCYSTDSSGNVFGTGTKVTVL | | | | | | |
| | COV2- | 1589 | EIVMTQSPATLSVSAGERATLSCRASQ | IGHV2-5 | IGHJ3 | IGKV3-15 | IGKJ4 | 2598 | AHRLWFRDA |
| | 2614 | | SISSNLAWYHQKPGQAPRLLIYGASTR | (Human) | (Human) | (Human) | (Human) | | FDI |
| | | | ATGIPARFSGSGSGTEFTLTISSLQSED | | | | | | |
| | | | FAVYYCQQYNSYSLTFGGGTKVEIK | | | | | | |
| | COV2- | 1590 | EIVLTQSPGTLSLSPGERATLSCRASQS | IGHV3-72 | IGHJ4 | IGKV3-20 | IGKJ3 | 2599 | ASVITFGGVIV |
| | 2616 | | VSSSYLAWYQQKPGQAPRLLIYGASSR | (Human) | (Human) | (Human) | (Human) | | RSY |
| | | | ATGIPDRFSGSGSGTDFTLTISRLEPED | | | | | | |
| | | | FAVYYCQYYGSSPFGFGPGTKVDIK | | | | | | |
| | COV2- | 1591 | EIVLTQSPATLSLSPGERATLSCRASQS | IGHV1-69 | IGHJ4 | IGKV3-11 | IGKJ4 | 2600 | ARVSGYGDY |
| | 2617 | | VSRFLAWYQQKPGQAPRLLIYDASNR | (Human) | (Human) | (Human) | (Human) | | GAYSDY |
| | | | ATGIPARFSGSGSGTDFTLTISSLEPED | | | | | | |
| | | | FAVYYCQQRSNWPPRLTFGGGTKVEIK | | | | | | |
| | COV2- | 1592 | QSVLTQPPSASGTPGQRVTISCSGSSS | IGHV3-20 | IGHJ4 | IGLV1-44 | IGU2 | 2601 | ASVITFGGVIV |
| | 2618 | | NIGSNTVNWYQQLPGTAPKLLIYSNN | (Human) | (Human) | (Human) | (Human) | | RSY |
| | | | QRPSGVPDRFSGSKSGTSASLAISGLQ | | | | | | |
| | | | SEDENNYYCAVWDDSLNGVVFGGGT | | | | | | |
| | | | KLTVL | | | | | | |
| | COV2- | 1593 | QSVLTQPPSASGTPGQRVTISCSGSSS | IGHV3-23 | IGHJ6 | IGLV1-47 | IGU2 | 2602 | ARVEGDWLL |
| | 2619 | | NIGSNYVYWYQQLPGTAPKLLISRNN | (Human) | (Human) | (Human) | (Human) | | GGPYYHYYG |
| | | | QRPSGVPDRFSGSKSGTSASLAISGLQ | | | | | | MDV |
| | | | SEDEANYYCLVWDDSLNGLVFGGGTK | | | | | | |
| | | | LTVL | | | | | | |
| | COV2- | 1594 | QSALTQPASVSGSPGQSITISCTGTSSD | IGHV3- | IGHJ4 | IGLV2-14 | IGU2 | 2603 | ARADTMVRG |
| | 2620 | | VGGYNYVSWYQQHPGKAPKLMIYDV | 303 | (Human) | (Human) | (Human) | | TYFEY |
| | | | SNRPSGVSNRFSGSKSGNTASLTISGL | (Human) | | | | | |
| | | | QAEDEADYCCSSYTSSRAVLFGGGTKL | | | | | | |
| | | | TVL | | | | | | |
| | COV2- | 1595 | DFVMTQSPGSLAVSLGERATINCRSSQ | IGHV3-33 | IGHJ4 | IGKV4-1 | IGKJ1 | 2604 | ARDYCNGVT |
| | 2621 | | SVLDNSSNKNHLAWHQQKPGQPPKL | (Human) | (Human) | (Human) | (Human) | | CNSNY |
| | | | LIYWASTRESGVPDRFSGSGSGTDFTL | | | | | | |
| | | | TISSLQAEDVAVYYCQQYYSSHWTFG | | | | | | |
| | | | QGTKVEIK | | | | | | |
| | COV2- | 1596 | QSALTQPASVSGSPGQSITISCTGTSSD | IGHV4-4 | IGHJ4 | IGLV2-23 | IGU3 | 2605 | ARGWYFDY |
| | 2622 | | VGSYNLVSWYQQHPGKAPKLMIYEVS | (Human) | (Human) | (Human) | (Human) | | |
| | | | KRPSGVSNRFSGSKSGNTASLTISGLQ | | | | | | |
| | | | AEDEADYYCYSYAGSSTWVFGGGTKL | | | | | | |
| | | | TVL | | | | | | |
| | COV2- | 1597 | SYELTQPPSVSVSPGQTARITCSADALP | IGHV1-69 | IGHJ6 | IGLV3-25 | IGU2 | 2606 | ARVEGEGVD |
| | 2624 | | KQYAYWYQQKPGQAPVLVIYKDSERP | (Human) | (Human) | (Human) | (Human) | | SYYYGMDV |
| | | | SGIPERFSGSSSGTTVTLTISGVQAEDE | | | | | | |
| | | | ADYYCQSTDSSGSYVVFGGGTKLTVL | | | | | | |
| | COV2- | 1598 | DIQMTQSPSSLSASVGDRVTITCRASQ | IGHV3-9 | IGHJ6 | IGKV1-39 | IGKJ2 | 2607 | AKDIIRQGED |
| | 2628 | | NIASYLNWYQQKPGKAPKLLIYAASSL | (Human) | (Human) | (Human) | (Human) | | GMDV |
| | | | QSGVPSRFSGSGSGTDFTLTISSLQPEE | | | | | | |
| | | | FATYYCHQSYFTPQTFGQGTKLEIK | | | | | | |
| | COV2- | 1599 | EIVLTQSPATLALSVGERATLSCVASQR | IGHV4-39 | IGHJ5 | IGKV3D-20 | IGKJ3 | 2608 | ARHPVDGYN |
| | 2631 | | VSSDYIAWYQKKPGLAPRLLVYHGSA | (Human) | (Human) | (Human) | (Human) | | YGYSDL |
| | | | WATGSSARFSGSGSGTAFTLTISSLEPE | | | | | | |
| | | | DFAVYYCQQYGNSPFTFGPGTKVEFK | | | | | | |
| | COV2- | 1600 | EIVLTQSPGTLSLSPGERATLSCRASQS | IGHV1-2 | IGHJ4 | IGKV3-20 | IGKJ2 | 2609 | ARVVVLGYGR |
| | 2632 | | VSSSYLAWYQEKPGQAPRLLMYSASS | (Human) | (Human) | (Human) | (Human) | | PNNYYDGRN |
| | | | RATGIPDRFSGSGSATDFTLTINRLEPE | | | | | | VWDY |
| | | | DFAVYYCQQYVEPPFTFGQGTKLEIK | | | | | | |
| | COV2- | 1601 | QSALTQPRSVSGSPGQSVTISCTGTSS | IGHV3-30 | IGHJ4 | IGLV2-11 | IGU2 | 2610 | ARAGGGSYR |
| | 2639 | | DVGGYNYVSWYQQHPGKAPKLMIYD | (Human) | (Human) | (Human) | (Human) | | GPFDY |
| | | | VSKRPSGVPDRFSGSKSGNTASLTVSG | | | | | | |
| | | | LQAEDEAEYYCSAYAGSNNLVFGGGT | | | | | | |
| | | | KLTVL | | | | | | |
| | COV2- | 1602 | EIVLTQSPGTLSLSPGERATLSCRAGQT | IGHV3-30 | IGHJ3 | IGKV3-20 | IGKJ2 | 2611 | AKSYNGNYYD |
| | 2641 | | VSSSYLAWYQHKPGQAPRLLIYGASSR | (Human) | (Human) | (Human) | (Human) | | AFDI |
| | | | ATGIPDRFSGSGSGTDFTLTISRLEPED | | | | | | |
| | | | FAVYYCQQYGSSYTFGQGTKLEIK | | | | | | |
| | COV2- | 1603 | DIQMTQSPSSLSASVGDRVTITCRASQ | IGHV3-30 | IGHJ6 | IGKV1-39 | IGKJ5 | 2612 | ARGSAGNYYY |
| | 2643 | | SISSYLNWYQQKPGKAPKLLIYAASSLQ | (Human) | (Human) | (Human) | (Human) | | GMDV |
| | | | SGVPSRFSGSGSGTDFTLTISSLQPEDY | | | | | | |
| | | | ATYYCQQSYSTPGTFGQGTRLEIK | | | | | | |
| | COV2- | 1604 | EIVLTQSPGTLSLSPGERATLSCRASQS | IGHV5-51 | IGHJ6 | IGKV3-20 | IGKJ1 | 2613 | ARLTFGGSGS |
| | 2656 | | VSSSYLAWYQQKPGQAPRLLIYGASSR | (Human) | (Human) | (Human) | (Human) | | YYFYYNGMD |
| | | | ATGIPDRFSGSGSGTDFTLTISRLEPED | | | | | | V |
| | | | FAVYYCQQYGRSSGTFGQGTKVEIK | | | | | | |
| | COV2- | 1605 | EIVLTQSPGTLSLSPGERATLSCRASQS | IGHV3-13 | IGHJ6 | IGKV3-20 | IGKJ5 | 2614 | ARADPYQLLG |
| | 2660 | | VSSSYLAWYQQKPGQAPRLLIYGASSR | (Human) | (Human) | (Human) | (Human) | | QHYYYGMDV |
| | | | ATGIPDRFSGSGSGTDFTLTISRLEPED | | | | | | |
| | | | FAVYYCQQYGSSPLITFGQGTRLEIK | | | | | | |
| | COV2- | 1606 | DIVMTQSPDFLAVSLGERATISCKSSQS | IGHV3-30 | IGHJ4 | IGKV4-1 | IGKJ4 | 2615 | AKGGDGSG |
| | 2669 | | VLYTPKNKNYLAWYKQKPGQPPKVLIY | (Human) | (Human) | (Human) | (Human) | | WAWDGDNP |
| | | | WASTRESGVPDRFSGSGSGTDFTLIISS | | | | | | PTDY |
| | | | LQAEDAAVYYCQQYYTAPLTFGGGTR | | | | | | |
| | | | VEIK | | | | | | |
| | COV2- | 1607 | QTVVTQEPSLTVSPGGTVTLTCASSAG | IGHV2-26 | IGHJ5 | IGLV7-43 | IGU3 | 2616 | ARIVLGASGT |
| | 2673 | | AVTSGYYPNWFQQKPGQAPRALIYST | (Human) | (Human) | (Human) | (Human) | | YPSPGFDP |
| | | | ANKHSWTPARFSGSLLGGKAALTLSG | | | | | | |
| | | | VQPEDEAEYYCLLYYGGAWVFGGGTK | | | | | | |
| | | | LTVL | | | | | | |
| | COV2- | 1608 | EIVLTQSPDFQSVTPKEKVTITCRASQN | IGHV3-30 | IGHJ5 | IGKV6-21 | IGKJ4 | 2617 | AKDGSGSYYG |
| | 2675 | | IGSSLHWYQQKPDQSPKVIIKYASQSF | (Human) | (Human) | (Human) | (Human) | | WFDP |
| | | | SGVPSRFSGSGSGTDFTLTINSLEAEDA | | | | | | |
| | | | ATYYCHQSSSLPPTFGGGTKVEIK | | | | | | |
| | COV2- | 1609 | EIVLTQSPATLSLSPGERATLSCRASQS | IGHV1-69 | IGHJ4 | IGKV3-11 | IGKJ3 | 2618 | ARSCGDCYSA |
| | 2676 | | ISSFLAWYQQKPGQAPRLLIYDASNRA | (Human) | (Human) | (Human) | (Human) | | DLDF |
| | | | TGIPARFSGSGSGTDFTLTISSLEPEDF | | | | | | |
| | | | AVYYCQRRSNWPPFTFGPGTKVDIK | | | | | | |
| | COV2- | 1610 | NFMLTQPHSVSESPGKTVTISCTGSSG | IGHV4-39 | IGHJ4 | IGLV6-57 | IGU3 | 2619 | ARLLWLRGHF |
| | 2677 | | SIASNYVQWYQQRPGSAPTTVIYEDN | (Human) | (Human) | (Human) | (Human) | | DY |
| | | | QRPSGVPDRFSGSIDSSSNSASLTISGL | | | | | | |
| | | | KTEDEADYYCQSYDSSNYWVFGGGTK | | | | | | |
| | | | LTVL | | | | | | |
| | COV2- | 1611 | SSELTQDPAVSVALGQTVRITCQGDSL | IGHV3-20 | IGHJ3 | IGLV3-19 | IGU3 | 2620 | AVIMSPIPRYS |
| | 2678 | | RSYYASWYQQKPGQVPILVIYDKNNR | (Human) | (Human) | (Human) | (Human) | | GYDWAGGAF |
| | | | PSGIPDRFSGSSSGNTASLTITGAQAED | | | | | | DI |
| | | | EADYYCNSRDSSGNAVVFGGGTKLTV | | | | | | |
| | | | L | | | | | | |
| | COV2- | 1612 | DIVMTQSPDSLAVSLGERATINCKSSQ | IGHV3-30 | IGHJ5 | IGKV4-1 | IGKJ2 | 2621 | ARALNKGFDP |
| | 2681 | | SVLYSSNNKNYLAWYQQKPGQPPKLLI | (Human) | (Human) | (Human) | (Human) | | |
| | | | YWASTRESGVPDRFSGSGSGTDFTLTI | | | | | | |
| | | | SSLQAEDVAVYYCQQYYSSPYTFGQGT | | | | | | |
| | | | KLEIK | | | | | | |
| | COV2- | 1613 | EIVLTQSPGTLSLSPGERATLSCRASQS | IGHV1-58 | IGHJ3 | IGKV3-20 | IGKJ1 | 2622 | AAPHCNRTSC |
| | 2684 | | VSSSYLGWYQQKPGQAPRLLIYGASSR | (Human) | (Human) | (Human) | (Human) | | YDAFDL |
| | | | ATGIPDRFSGSGSGTDFTLTISRLESED | | | | | | |
| | | | FALYYCQQYNNWWRTFGQGTKVEIK | | | | | | |
| | COV2- | 1614 | EIVLTQSPGTLSLSPGERATLSCRASQS | IGHV1-46 | IGHJ6 | IGKV3-20 | IGKJ4 | 2623 | ARDRLGDGSY |
| | 2685 | | VSSSYLAWYQQKRGQAPRLLIYGASSR | (Human) | (Human) | (Human) | (Human) | | LGGGYYGMD |
| | | | ATGIPDRFSGSGSGTDFTLTISRLEPED | | | | | | V |
| | | | FAVYYCQQYGSSPRLTFGGGTKVEIK | | | | | | |
| | COV2- | 1615 | QSVLTQPPSASGTPGQRVTISCSGSNS | IGHV1-8 | IGHJ5 | IGLV1-44 | IGU2 | 2624 | ARMRSGWPT |
| | 2693 | | NIGSYTVNWYQQLPGTAPKLLIYDNN | (Human) | (Human) | (Human) | (Human) | | HGRPDDL |
| | | | QRTSGVPDRLSGSKSGTSASLAISGLQS | | | | | | |
| | | | EDEANYYCAVWDDSLNGLVFGGGTK | | | | | | |
| | | | LTVL | | | | | | |
| | COV2- | 1616 | QSVLTQPPSVSGAPGQRVTISCTGSSS | IGHV1-69 | IGHJ4 | IGLV1-40 | IGU3 | 2625 | ARDHSGYYDS |
| | 2694 | | NIGAGYDVHWYQQLPGTAPKLLIFGN | (Human) | (Human) | (Human) | (Human) | | TSLMSPFFDY |
| | | | TNRPSGVPDRFSGSKSGTSTSLAITGLQ | | | | | | |
| | | | AEDEADYYCQSYDSSLNGDVFGGGTK | | | | | | |
| | | | LTVL | | | | | | |
| | COV2- | 1617 | EIVLTQSPATLSLSPGERATLSCRASQS | IGHV1-46 | IGHJ5 | IGKV3-11 | IGKJ3 | 2626 | ARDLAGVPA |
| | 2697 | | VSSYLAWYQQKPGQAPRLLIYDAYKR | (Human) | (Human) | (Human) | (Human) | | ALGCWFDP |
| | | | DTGIPARFSGSGSGTDFTLTISSLEPED | | | | | | |
| | | | FAVYYCQQRSNWPLIFTFGPGTKVDIK | | | | | | |
| | COV2- | 1618 | QSVLTQPPSVSGVPGQRVTVSCTGSSS | IGHV3-30 | IGHJ4 | IGLV1-40 | IGU1 | 2627 | AKNLGPYCSG |
| | 2700 | | NIGAGFDVYWYQQFLGTAPKLLIYGN | 3 | (Human) | (Human) | (Human) | | GTCYSLVGDY |
| | | | NNRPSGVPDRFSASKSGTSASLAITGL | (Human) | | | | | |
| | | | QAEDEADYYCQSYDSSLSGYVFGTGTK | | | | | | |
| | | | VTVL | | | | | | |
| | COV2- | 1619 | DIQMTQSPSSLSASVGDRVTITCRASQ | IGHV3-13 | IGHJ4 | IGKV1-39 | IGKJ5 | 2628 | ARARGGYNW |
| | 2703 | | SISSYLNWYQQKPGKAPKLLIYDASSLQ | (Human) | (Human) | (Human) | (Human) | | NFDY |
| | | | SGVPSRFSGSGSGTDFTLTISSLQPEDF | | | | | | |
| | | | ATYYCQQSYSTPPITFGQGTRLEIK | | | | | | |
| | COV2- | 1620 | DIQMTQSPSSLSTSVGDRVTITCRASQ | IGHV3-30 | IGHJ4 | IGKV1-39 | IGKJ1 | 2629 | ARDQGTVVT |
| | 2705 | | SIRSYLNWYQQKPGKAPKLLIYVASSL | (Human) | (Human) | (Human) | (Human) | | HFDY |
| | | | QSGVPSRFSGSGSGTDFTLTISSLQPED | | | | | | |
| | | | FATYYCQQSYSTPPWTFGQGTKVEIK | | | | | | |
| | COV2- | 1621 | DIQMTQSPSSLSASVGDRVTITCQASQ | IGHV3-30 | IGHJ4 | IGKV1-33 | IGKJ3 | 2630 | AKNLGPYCSG |
| | 2709 | | DISNYLNWYQQKPGKAPKLLIYDASNL | (Human) | (Human) | (Human) | (Human) | | GTCYSLVGDY |
| | | | ETGVPSRFSGSGSGTDFTFTISSLQPED | | | | | | |
| | | | IATYYCQQYANLPFTFGPGTKVDIK | | | | | | |
| | COV2- | 1622 | QSVLTQPPSASGTPGQRVTISCSGSNS | IGHV1-8 | IGHJ4 | IGLV1-44 | IGU2 | 2631 | ARMRSGWPT |
| | 2710 | | NIGSYTVNWYQQLPGTAPKLLIFGNN | (Human) | (Human) | (Human) | (Human) | | HGRPDDH |
| | | | QRPSGVPDRFSGSKSGTSASLAISGLQ | | | | | | |
| | | | SEDEADYYCVAWDDSRNGLVFGGGA | | | | | | |
| | | | KLTVL | | | | | | |
| | COV2- | 1623 | SYELTQPPSVSVSPGQTARITCSTDALP | IGHV3-43 | IGHJ6 | IGLV3-25 | IGU2 | 2632 | AKDEMAYPP |
| | 2713 | | NEYIYWYQQKPGQAPVLVIYKDSERPS | (Human) | (Human) | (Human) | (Human) | | SHHYYYYYMD |
| | | | GIPERFSGSSSGTTVTLTISGVQAEDEA | | | | | | V |
| | | | DYYCQSVDSSGTYPHVIFGGGTKLTVL | | | | | | |
| | COV2- | 1624 | DIQMTQSPSSLSASVGDRVTITCRASQ | IGHV1-8 | IGHJ4 | IGKV1-39 | IGKJ2 | 2633 | ARGPSILTGFY |
| | 2717 | | SIISYLNWYHQKPGKAPKLLIYAASSLQ | (Human) | (Human) | (Human) | (Human) | | NPLDY |
| | | | SGVPSRFSGSGSGTDFTLTISSLQPEDF | | | | | | |
| | | | ATYYCQQSYSTPYTFGQGTKLEIK | | | | | | |
| | COV2- | 1625 | DIQLTQSPSFLSASVGDRVTITCRASQG | IGHV4-39 | IGHJ6 | IGKV1-9 | IGKJ4 | 2634 | ARRTYYDLWS |
| | 2718 | | ISSYLAWYQQKPGKAPKLLIYAASTLQS | (Human) | (Human) | (Human) | (Human) | | AYSSTAYYCM |
| | | | GVPSRFSGSGSGTEFTLTISSLQPEDFA | | | | | | DV |
| | | | TYYCQQLNSYPLTFGGGTKVEIK | | | | | | |
| | COV2- | 1626 | QSALTQPASVSGSPGQSITISCTGTSSD | IGHV1-69 | IGHJ6 | IGLV2-14 | IGU2 | 2635 | ARLSGSGWL |
| | 2722 | | VGGYNFVSWYQQHPGKAPKLIIYDVS | (Human) | (Human) | (Human) | (Human) | | GYAMDV |
| | | | NRPSGVSNRFSGSKSGNTASLTISGLQ | | | | | | |
| | | | AEDEADYYCTSYTSSSTLNVVFGGGTK | | | | | | |
| | | | LTVL | | | | | | |
| | COV2- | 1627 | QSALTQPASVSGSPGQSITISCTGTSSD | IGHV3-30 | IGHJ2 | IGLV2-23 | IGU2 | 2636 | ARPSNWYFD |
| | 2726 | | VGSYNLVSWYQQHPGKAPKLMIYEVS | (Human) | (Human) | (Human) | (Human) | | L |
| | | | KRPSGVSNRFSGSKSGNTASLTISGLQ | | | | | | |
| | | | AEDEADYYCCSYASSSIVVFGGGTKLTV | | | | | | |
| | | | L | | | | | | |
| | COV2- | 1628 | DIQMTQSPSSLSASVGDRVTITCRASQ | IGHV3-9 | IGHJ4 | IGKV1-39 | IGKJ1 | 2637 | AKAGYYAYV |
| | 2730 | | SISSYLNWYQQKPGKAPKLLLYAASSL | (Human) | (Human) | (Human) | (Human) | | WGSYRFEYFD |
| | | | QSGVPSRFSGSGSGTDFTLTISSLQPED | | | | | | N |
| | | | FATYYCQQSYSTPPWTFGQGTKVEIK | | | | | | |
| | COV2- | 1629 | DIQLTQSPSFLSASVGDRVTITCRASQG | IGHV3-53 | IGHJ3 | IGKV1-9 | IGKJ3 | 2638 | ARGPEPDAFD |
| | 2733 | | ISSYLAWYQQKPGKAPKLLIYAASTLQS | (Human) | (Human) | (Human) | (Human) | | |
| | | | GVPSRFSGSGSGTEFTLTISSLQPEDFA | | | | | | |
| | | | TYYCQQLNSYSFETFGPGTKVAIK | | | | | | |
| | COV2- | 1630 | NFMLTQPHSVSESPGKTVTFSCTGSSG | IGHV4-61 | IGHJ4 | IGLV6-57 | IGU2 | 2639 | AGSPVPPTIV |
| | 2734 | | SIASNYVQWYQQRPGSAPTTVIYEDN | (Human) | (Human) | (Human) | (Human) | | GASY |
| | | | QRPSGVPDRFSGSIDSSSNSASLTISGL | | | | | | |
| | | | KTEDEADYYCQSYDGINRWLVFGGGT | | | | | | |
| | | | KLTVL | | | | | | |
| | COV2- | 1631 | DIQMTQSPSTLSASIGDRVTITCRASQS | IGHV3- | IGHJ3 | IGKV1-5 | IGKJ2 | 2640 | ARDLSTTWYL |
| | 2736 | | ISSWLAWYQQIPGKAPKLLIYKASSLES | 303 | (Human) | (Human) | (Human) | | EMWGPDAF |
| | | | GVPSRFSGSGSGTEFTLTISSLQPDDFA | (Human) | | | | | DI |
| | | | TYYCQQYNSYPYTFGQGTKLEIK | | | | | | |
| | COV2- | 1632 | DIQMTQSPSSLSASIGDRVTITCRASQ | IGHV3-30 | IGHJ4 | IGKV1-39 | IGKJ2 | 2641 | AKKGGPYCG |
| | 2740 | | NIRSYLNWYQHKPGKAPKLLIYAASTL | (Human) | (Human) | (Human) | (Human) | | GGNCYAGYF |
| | | | QSGVPSRFSGSESGTDFTLTISSLQPED | | | | | | DY |
| | | | FATYYCQQSSSSPITFGQGTKLEIK | | | | | | |
| | COV2- | 1633 | DIQMTQSPSSLSASVGDRVTITCRASQ | IGHV4-34 | IGHJ6 | IGKV1-39 | IGKJ4 | 2642 | ARVGYSQGYY |
| | 2749 | | SISNYLNWYQQKPGKAPKLLIYAASSL | (Human) | (Human) | (Human) | (Human) | | YYYMDV |
| | | | QSGVPSRFSGSGSGTDFSLTISSLQPED | | | | | | |
| | | | FATYSCQQSYTTLLTFGGGTKVEIK | | | | | | |
| | COV2- | 1634 | SYELAQPPSVSVAPGETATIFCRATYIG | IGHV3-9 | IGHJ6 | IGLV3-21 | IGU2 | 2643 | ARDYCSSTTC |
| | 2751 | | RKNVQWYQQKPGQAPVLVVYDDSD | (Human) | (Human) | (Human) | (Human) | | PAETYYYMDV |
| | | | RPSGIPGRFSGSNSGDTATLTISRIEAG | | | | | | |
| | | | DEAAYYCQVWDGINDRVVFGGGTKL | | | | | | |
| | | | TVL | | | | | | |
| | COV2- | 1635 | DIQMTQSPSSLSASVGDRVTITCQASQ | IGHV3-53 | IGHJ5 | IGKV1-33 | IGKJ4 | 2644 | ARDFLRWHD |
| | 2752 | | DINNYLNWYQQKPGKAPKLLIYDASN | (Human) | (Human) | (Human) | (Human) | | L |
| | | | LETGVPLRFSGSGSGTDFTFTISSLQPE | | | | | | |
| | | | DIATYYCQQYDNLPPVFGGGTKVEIK | | | | | | |
| | COV2- | 1636 | DFQMTQSPSSLSASVGDRVTITCRASQ | IGHV3-13 | IGHJ4 | IGKV1-39 | IGKJ1 | 2645 | ARVDFDILTG |
| | 2753 | | SISTYLNWYQQKPGKAPNLLIFAASSLH | (Human) | (Human) | (Human) | (Human) | | YYSN |
| | | | SGVPSRFSGSGSGTDFTLSISSLQPEDF | | | | | | |
| | | | ATYYCQQSYSSEWTFGQGTKVEIK | | | | | | |
| | COV2- | 1637 | QSLLTQPPSVSGAPGQRVTLSCAGATS | IGHV3-23 | IGHJ4 | IGLV1-40 | IGU3 | 2646 | AKWAGPIVM |
| | 2756 | | NIGAGSDVHWYQQLPGTAPKLLIYYN | (Human) | (Human) | (Human) | (Human) | | KYYLQY |
| | | | TNRPSGVPDRFSGSKSATSASLVITGL | | | | | | |
| | | | QTEDEADYYCQSYDISLGGWVFGGGT | | | | | | |
| | | | KLTVL | | | | | | |
| | COV2- | 1638 | DIQMTQSPSSLSASVGDRVTITCRASQ | IGHV3-13 | IGHJ2 | IGKV1-39 | IGKJ5 | 2647 | ARGGDSGYD |
| | 2758 | | SVSIYLNWYQQKPGKAPKLLIYAASSL | (Human) | (Human) | (Human) | (Human) | | LGAWYFDL |
| | | | QSGVPSRFSGSGSGTDFTLTVSSLQPE | | | | | | |
| | | | DFATYYCQQSYSMPPITFGQGTRLEIK | | | | | | |
| | COV2- | 1639 | QSVLTQPPSASGTPGQRVTISCSGSSS | IGHV5-51 | IGHJ5 | IGLV1-44 | IGU3 | 2648 | ARTPTLYNWF |
| | 2759 | | NIGSNSLNWYQQLPGTAPKLLIYSNN | (Human) | (Human) | (Human) | (Human) | | HP |
| | | | QRPSGVPDRFSGSKSGTSASLAISGLQ | | | | | | |
| | | | SEDEADYYCAAWDDSLNGSWVFGGG | | | | | | |
| | | | TKLTVL | | | | | | |
| | COV2- | 1640 | QSVLTQPPSASGTPGQRVTISCSGSRY | IGHV2-70 | IGHJ6 | IGLV1-47 | IGU2 | 2649 | ARATTFFYGM |
| | 2760 | | NIGSNYVYWYQQLPGTAPRLLIYSNN | (Human) | (Human) | (Human) | (Human) | | DV |
| | | | QRPSGVPDRFSGSKSGTSASLAISGLRS | | | | | | |
| | | | EDEADYYCAAWDDSLSGLIFGGGTKLT | | | | | | |
| | | | VQ | | | | | | |
| | COV2- | 1641 | QSALTQPASVSGSPGQSITISCTGTSSD | IGHV4-39 | IGHJ4 | IGLV2-23 | IGU3 | 2650 | ARHQRYCSSS |
| | 2762 | | VGSYNLVSWYQQHPGKAPKLMIYEGS | (Human) | (Human) | (Human) | (Human) | | SCHVWDY |
| | | | KRPSGVSNRFSGSKSGNTASLTISGLQ | | | | | | |
| | | | AEDEADYYCCSYAGSSTWLFGGGTKLT | | | | | | |
| | | | VL | | | | | | |
| | COV2- | 1642 | SYVLTQPPSVSVAPGKTARITCEGNNI | IGHV3-9 | IGHJ6 | IGLV3-21 | IGU2 | 2651 | AKGRGAGYTS |
| | 2765 | | GSKSVHWYQQKPGQAPVLVVYDDSG | (Human) | (Human) | (Human) | (Human) | | YMDV |
| | | | RPSGIPERFSGSNSGNTATLTISRVEAG | | | | | | |
| | | | DEADYFCQVWDSSSDHHVVFGGGTK | | | | | | |
| | | | LTVL | | | | | | |
| | COV2- | 1643 | QSVLTQPPSVSGAPGQRVTISCTGSSS | IGHV3-53 | IGHJ3 | IGLV1-50 | IGU2 | 2652 | ARSYDILTGYR |
| | 2767 | | NIGAGYDVHWYQQLPETAPKLLIYAN | (Human) | (Human) | (Human) | (Human) | | DAFDI |
| | | | SNRPSGVPDRFSGSKSGTSASLAISGL | | | | | | |
| | | | QSEAEADYYCVAWDDSRNGLVFGGG | | | | | | |
| | | | AKLTVL | | | | | | |
| | COV2- | 1644 | QSVLTQPPSVSAAPGQKVTISCSGSSS | IGHV3- | IGHJ6 | IGLV1-51 | IGU1 | 2653 | ARDDNSPQG |
| | 2768 | | NIGNNYVSWYQQLPGTAPKLLIYDNN | 303 | (Human) | (Human) | (Human) | | SGWYFYYYYA |
| | | | ERPSGIPDRFSGSKSGTSATLGITGLQT | (Human) | | | | | MDV |
| | | | GDEADYYCGTWDSSLSAYVFGTGTKV | | | | | | |
| | | | TVL | | | | | | |
| | COV2- | 1645 | SYVLTQPPSVSVAPGKTARITCGGNNI | IGHV3- | IGHJ3 | IGLV3-21 | IGU3 | 2654 | ARDWAPTYY |
| | 2769 | | GNKGVHWYQQKPGQAPVLVVDDDS | 303 | (Human) | (Human) | (Human) | | DMPSAFDI |
| | | | DRPSGIPERFSGSNSGNTATLIISSVEV | (Human) | | | | | |
| | | | GDEADFYCQVWDSSSDHPGVFGGGT | | | | | | |
| | | | KLTVL | | | | | | |
| | COV2- | 1646 | SYELTQPPSVSVSPGQTASITCSGDKLG | IGHV3-7 | IGHJ4 | IGLV3-1 | IGU3 | 2655 | VRLGVSSWYF |
| | 2774 | | DKYACWYQQKPGQSPVLVIYQDTKRP | (Human) | (Human) | (Human) | (Human) | | DY |
| | | | SGIPERFSGSNSGNTATLTISGTQAMD | | | | | | |
| | | | EADYYCQAWGSSRGVFGGGTKLTVL | | | | | | |
| | COV2- | 1647 | SSELTQDPAVSVALGQTVRITCQGDSL | IGHV3-7 | IGHJ6 | IGLV3-19 | IGU3 | 2656 | ARWVEVAT |
| | 2776 | | RSYYASWYQQKPGQAPVVVIYGKNN | (Human) | (Human) | (Human) | (Human) | | NKGIHGVDYY |
| | | | RPSGIPDRFSGSSSGNKASLTITGAQA | | | | | | YYYYMDV |
| | | | GDEADYYCNSRDNSGNLNWVFGGGT | | | | | | |
| | | | KLTVL | | | | | | |
| | COV2- | 1648 | DIQMTQSPSSLSASVGDRVTITCQASQ | IGHV4-34 | IGHJ4 | IGKV1-33 | IGKJ2 | 2657 | ARLRYSSSGG |
| | 2780 | | DINNYLNWYQQKSGKAPKLLIYDASNL | (Human) | (Human) | (Human) | (Human) | | HIFDY |
| | | | ETGVPSRFSGSGSGTDFTFTINSLQPED | | | | | | |
| | | | IATYYCQQYDNLPYTFGQGTKLEIK | | | | | | |
| | COV2- | 1649 | SYELTQPPSVSVSPGQTARITCSGDALP | IGHV1-69 | IGHJ4 | IGLV3-25 | IGU2 | 2658 | ARGLTGSSAY |
| | 2783 | | KQYAYWYQQKPGQAPVLVIYKDTERP | (Human) | (Human) | (Human) | (Human) | | KDEIYFDY |
| | | | SGIPERFSGSSSGTTVTLTISGVQAEDE | | | | | | |
| | | | ADYYCQSADSSGSRFGGGTKLTVL | | | | | | |
| | COV2- | 1650 | EIVMTQSPATLSVSPGERATLSCRASQ | IGHV4-59 | IGHJ6 | IGKV3-15 | IGKJ2 | 2659 | ARDGGNAYS |
| | 2784 | | SVSSNLAWYQQKPGQAPRLLIYGAST | (Human) | (Human) | (Human) | (Human) | | SGWYRYYYH |
| | | | RATVIPARFSGSGSGTEFTLTISSLQSE | | | | | | MDV |
| | | | DFAVYYCQQYNNWPYTFGQGTKLEIK | | | | | | |
| | COV2- | 1651 | DIQMTQSPSSLSASVGDRVTITCRASQ | IGHV3-13 | IGHJ5 | IGKV1-39 | IGKJ1 | 2660 | ARVLYDSSGF |
| | 2786 | | SISSYLNWYQQKPGKAPKLLIYAASSLE | (Human) | (Human) | (Human) | (Human) | | YNWFDP |
| | | | SGVPSRFSGSGSGTDFTLTISSLQPEDF | | | | | | |
| | | | ATYYCQQSYEIPPWTFGQGTKVEIK | | | | | | |
| | COV2- | 1652 | QSVLTQPPSASGTPGQRVTISCSGSSS | IGHV1-46 | IGHJ5 | IGLV1-44 | IGU2 | 2661 | ARGFHVPAAL |
| | 2789 | | NIGSNTVNWYQQLPGTAPKLLIYNNN | (Human) | (Human) | (Human) | (Human) | | RNWFDP |
| | | | QRPSGVPDRLSGSKSGTSASLAISGLQ | | | | | | |
| | | | SEDEADYYCATWDDSLNGPVFGGGT | | | | | | |
| | | | KLTVL | | | | | | |
| | COV2- | 1653 | QSVLTQPPSVSGAPGQRVTISCTGSSS | IGHV3-53 | IGHJ3 | IGLV1-40 | IGU2 | 2662 | ARSYDILTGYR |
| | 2790 | | NIGSGSDVHWYQQLPGTAPKLLIYGN | (Human) | (Human) | (Human) | (Human) | | DAFDI |
| | | | TNRPSGVPDRFSGSKSGTSASLAITGL | | | | | | |
| | | | QAEDEADYYCQSYDSRLSGFVVFGGG | | | | | | |
| | | | TKLTVL | | | | | | |
| | COV2- | 1654 | SYELTQPPSVSVSPGQTARITCSGDALP | IGHV3-7 | IGHJ4 | IGLV3-25 | IGU2 | 2663 | ARVNDGRPN |
| | 2794 | | KQYAYWYHQKPGQAPVLVIYKDSERP | (Human) | (Human) | (Human) | (Human) | | PLEYYFDY |
| | | | SGIPERFSGSSSGTTVTLTISGVQAEDE | | | | | | |
| | | | ADYYCQSADSSGTSVLFGGGTKLTVL | | | | | | |
| | COV2- | 1655 | QSALTQPASVSGSPGQSITISCTGTSSD | IGHV4-31 | IGHJ4 | IGLV2-23 | IGU3 | 2664 | ARETYSAYEM |
| | 2796 | | VGSYNLVSWYQQHPGKAPKLMIYEGS | (Human) | (Human) | (Human) | (Human) | | PPYFDY |
| | | | KRPSGVSNRFSGSKSGNTASLTISGLQ | | | | | | |
| | | | AEDEADYYCCSYARSSTRVFGGGTKLT | | | | | | |
| | | | VL | | | | | | |
| | COV2- | 1656 | SYELTQPPSVSVSPGQTARITCSGDAFP | IGHV5-51 | IGHJ6 | IGLV3-25 | IGU1 | 2665 | ARDLIIESTIAA |
| | 2797 | | KQYGYWYQQKPGQAPVLVIYKDSERP | (Human) | (Human) | (Human) | (Human) | | RPGYYGMDV |
| | | | SGIPERFSGSSSGTTVTLTISGVQAEDE | | | | | | |
| | | | ADYYCQSADSRGAVFGSGTKVTVL | | | | | | |
| | COV2- | 1657 | QSVLTQPPSASGTPGQSVTISCSGSSS | IGHV1-46 | IGHJ4 | IGLV1-44 | IGU1 | 2666 | ARERSGTYFF |
| | 2801 | | NIGNNKVNWYQQLPGTAPKVLIYNSN | (Human) | (Human) | (Human) | (Human) | | DY |
| | | | QRPSGVPDRFSGSKSGTSASLAISGLQ | | | | | | |
| | | | SEDEADYYCAVWDDSLHSYVFGTETK | | | | | | |
| | | | VTVL | | | | | | |
| | COV2- | 1658 | DIQMTQSPSSLSASVGDRVTITCRASQ | IGHV2-70 | IGHJ4 | IGKV1-39 | IGKJ1 | 2667 | ARETPVTAID |
| | 2807 | | SISRYLNWYQQKPGKAPKLLIYTASSLQ | (Human) | (Human) | (Human) | (Human) | | Y |
| | | | SGVPSRFSGSGSGTDFTLTISSLQPEDF | | | | | | |
| | | | ATYYCQQSYSTPRTFGQGTKVEIK | | | | | | |
| | COV2- | 1659 | DIQMTQSPSSLSASVGDRVTITCRASQ | IGHV1-58 | IGHJ3 | IGKV1-33 | IGKJ3 | 2668 | AAPYCSSISCN |
| | 2808 | | SISNYLNWYQQKPGKAPKLLIYDASNL | (Human) | (Human) | (Human) | (Human) | | DGFDI |
| | | | ETGVPSRFSGSGSGTDFTFTISSLQPDD | | | | | | |
| | | | IATYYCQQYDNLPLAFGPGTKVDIK | | | | | | |
| | COV2- | 1660 | DIQMTQSPSSLSASVGDRVTITCRASQ | IGHV1-3 | IGHJ6 | IGKV1-39 | IGKJ4 | 2669 | ASMTRMSEQ |
| | 2809 | | SISNYLNWYQQKPGKAPKLLIYAASSL | (Human) | (Human) | (Human) | (Human) | | TYYGMDV |
| | | | QSGVPSRFSGSGSGTDFSLTISSLQPED | | | | | | |
| | | | FATYSCQQSYTTLLTFGGGTKVEIK | | | | | | |
| | COV2- | 1661 | QSVLTQPPSVSGAPGQRVTISCTGSSS | IGHV3-30 | IGHJ4 | IGLV1-40 | IGU3 | 2670 | ARELMSVG |
| | 2811 | | NIGARYDVHWYQQLPGTAPKLLMYG | (Human) | (Human) | (Human) | (Human) | | |
| | | | NNNRPSGVPDRFSGSKSGTSVSLAITG | | | | | | |
| | | | LQAEDEADYYCQSYDSSLSGWVFGGG | | | | | | |
| | | | TKLTVL | | | | | | |
| | COV2- | 1662 | VIQMTQSPSSLSASVGDRVTITCQASQ | IGHV3-53 | IGHJ6 | IGKV1-33 | IGKJ3 | 2671 | ARDALYYNGP |
| | 2812 | | DINKYLNWYQQKPGKAPKLLIYDASNL | (Human) | (Human) | (Human) | (Human) | | GRDGMDV |
| | | | ETGVPSRFSGSGSGTDFTFTISSLQPED | | | | | | |
| | | | IATYYCQQYANLPFTFGPGTKVDIK | | | | | | |
| | COV2- | 1663 | VIQMTQSPSSLSASVGDRVTITCQASQ | IGHV3-53 | IGHJ6 | IGKV1-33 | IGKJ2 | 2672 | ARDALYYNGP |
| | 2813 | | DINKYLNWYQQKPGKAPKLLIYDASNL | (Human) | (Human) | (Human) | (Human) | | GRDGMDV |
| | | | ETGVPSRFSGSGSGTDFTFTISSLQPED | | | | | | |
| | | | IATYYCQQYDNLPRTFGQGTKLEIK | | | | | | |
| | COV2- | 1664 | QSVLTQPPSVSGAPGQRVTISCTGSSS | IGHV3-30 | IGHJ6 | IGLV1-40 | IGU1 | 2673 | AKDMVEPLFS |
| | 2814 | | NIGAGYDVHWYQQLPGTAPKLLIYGN | (Human) | (Human) | (Human) | (Human) | | HYYYYGMDV |
| | | | SNRPSGVPDRFSGSKSGTSASLAISGL | | | | | | |
| | | | QAEDEADYSCQSYDSSLSGYVFGTGTK | | | | | | |
| | | | VTVL | | | | | | |
| | COV2- | 1665 | QSALTQPASVSGSPGQSITISCTGTSSD | IGHV4-59 | IGHJ4 | IGLV2-23 | IGU2 | 2674 | ARAPRERLQ |
| | 2816 | | VGSYNLVSWYQQHAGKAPKLMIYEVI | (Human) | (Human) | (Human) | (Human) | | WGEYYFDY |
| | | | KRPSGVSNRFSGSKSGNTASLTISGLQ | | | | | | |
| | | | AEDEADYYCCSYAVSTTYVIFGGGTKLT | | | | | | |
| | | | VL | | | | | | |
| | COV2- | 1666 | DIQMTQSPSSLSASVGDRVTITCRASQ | IGHV3-13 | IGHJ6 | IGKV1-39 | IGKJ2 | 2675 | ARVFETKVIR |
| | 2817 | | SISSYLNWYQQKPGKAPKLLIYAASSLQ | (Human) | (Human) | (Human) | (Human) | | GGRYYYYYYM |
| | | | SGVPSRFSGSGSGTDFTLTISSLQPEDF | | | | | | DV |
| | | | ATYYCQQSYSNPSYTFGQGTKLEIK | | | | | | |
| | COV2- | 1667 | EIVLTQSPGTLSLSPGERATLSCRASQS | IGHV4-34 | IGHJ6 | IGKV3-20 | IGKJ2 | 2676 | ARCRQMGNF |
| | 2818 | | VASSYLAWYQQKPGQAPRLLIYGASG | (Human) | (Human) | (Human) | (Human) | | YYYYMDV |
| | | | RATGIPDRFSGSGSGTDFTLTISRLEPE | | | | | | |
| | | | DFAVYYCQQYGSSPPRYTFGQGTKLEI | | | | | | |
| | | | K | | | | | | |
| | COV2- | 1668 | DIQMTQSPSSLSASVGDRVTITCRASQ | IGHV4-31 | IGHJ5 | IGKV1-39 | IGKJ2 | 2677 | ARVVPTRGPV |
| | 2819 | | SISSYLNWYQQRPGKAPKLLIYAASSLQ | (Human) | (Human) | (Human) | (Human) | | AWFDP |
| | | | SGVPSRFSGSGSGTDFTLTISTLQPEDF | | | | | | |
| | | | ATYYCQQSYSTLLYTFGQGTKLEIK | | | | | | |
| | COV2- | 1669 | DIVMTQSPLSLPVTPGEPASISCRSSQS | IGHV3-33 | IGHJ6 | IGKV2-28 | IGKJ3 | 2678 | ARDQSQGAYI |
| | 2820 | | LLHSNGYNYLDWYLQKPGQSPQFLIYL | (Human) | (Human) | (Human) | (Human) | | LTGYRGYGM |
| | | | GSNRASGVPDRFSGSGSGTDFILKISR | | | | | | DV |
| | | | VEAEDVGVYYCMQALQTPFTFGPGTK | | | | | | |
| | | | VDIK | | | | | | |
| | COV2- | 1670 | SYVLTQPPSVSVAPGKTASITCEGNNI | IGHV1-69 | IGHJ4 | IGLV3-21 | IGU1 | 2679 | TTTQGGDYG |
| | 2821 | | GSKSVHWYQQKPGQAPVLVIYYDSDR | (Human) | (Human) | (Human) | (Human) | | DNLYYLDY |
| | | | PSGIPERFSGSNSGNTATLTISRVEAGD | | | | | | |
| | | | EADYYCQVWDSSSDRLYVFGTGTKVT | | | | | | |
| | | | VL | | | | | | |
| | COV2- | 1671 | DIQLTQSPSFLSASVGDRVTITCRASQG | IGHV3-53 | IGHJ3 | IGKV1-9 | IGKJ2 | 2680 | ARGPEPDAFD |
| | 2822 | | ISSYLAWYQQKPGKAPKLLIYAASTLQS | (Human) | (Human) | (Human) | (Human) | | |
| | | | GVPSRFSGSGSGTDFTLTISSLQPEDFA | | | | | | |
| | | | TYYCQQSYSNPSYTFGQGTKLEIK | | | | | | |
| | COV2- | 1672 | QSALTQPASVSGSPGQSITISCTGTSSD | IGHV1-46 | IGHJ4 | IGLV2-23 | IGU3 | 2681 | ARGYGFVPN |
| | 2826 | | VGSYNLVSWYQQHPGKAPKLMIYEGS | (Human) | (Human) | (Human) | (Human) | | VLYYFDY |
| | | | KRPSGVSNRFSGSKSGITASLTISGLQA | | | | | | |
| | | | EDEADYYCCSYAGSSTWLFGGGTKLTV | | | | | | |
| | | | L | | | | | | |
| | COV2- | 1673 | NFMLTQPHSVSESPGKTVTISCTGSSG | IGHV3-21 | IGHJ4 | IGLV6-57 | IGU2 | 2682 | ARGGSILWW |
| | 2828 | | SIASNYVQWYQQRPGSAPTTVIYEDN | (Human) | (Human) | (Human) | (Human) | | LIDY |
| | | | QRPSGVPDRFSGSIDSSSNSASLTISGL | | | | | | |
| | | | KTEDEADYYCQSYDSTSRVVFGGGTKL | | | | | | |
| | | | TVL | | | | | | |
| | COV2- | 1674 | SYVLTQPPSVSVAPGRTARITCGGNNI | IGHV3-23 | IGHJ3 | IGLV3-21 | IGU2 | 2683 | AKDSRSGIAG |
| | 2830 | | GSKSVHWYQQKPGQAPVLVVYDDSD | (Human) | (Human) | (Human) | (Human) | | VDAFDI |
| | | | RPSGIPERFSGSKFGNTATLIISRVEAG | | | | | | |
| | | | DEADYSCQVWDSGSDHVVFGGGTKV | | | | | | |
| | | | TVL | | | | | | |
| | COV2- | 1675 | DIQMTQSPSSLSASVGDRVTITCRASQ | IGHV3-66 | IGHJ4 | IGKV1-39 | IGKJ1 | 2684 | ARGDGGYYS |
| | 2832 | | SISSYLNWYQQKPGKAPKVLIYAASTM | (Human) | (Human) | (Human) | (Human) | | PFDY |
| | | | QSGVPSRFRGSGSGTDFTLTISSLQLED | | | | | | |
| | | | FATYYCQQSYSTPQTFGQGTKVEIK | | | | | | |
| | COV2- | 1676 | QSVLTQPPSVSEAPRQRVTISCSGGSS | IGHV2-5 | IGHJ5 | IGLV1-36 | IGU2 | 2685 | AHRLPTPQLL |
| | 2834 | | NIGNNAVNWYQQLPGKAPKLLIYYDD | (Human) | (Human) | (Human) | (Human) | | PSFENWFDP |
| | | | LLPSGVSDRFSGSKSGTSASLAISGLQS | | | | | | |
| | | | EDEADYYCASWDDSLIGPVFGGGTKL | | | | | | |
| | | | TVL | | | | | | |
| | COV2- | 1677 | DIQLTQSPSFLSASVGDRVTITCRASQG | IGHV3-66 | IGHJ4 | IGKV1-9 | IGKJ2 | 2686 | AREVVGYFDC |
| | 2835 | | ISTYLAWYQQKPGKAPKLLIYAASTLQS | (Human) | (Human) | (Human) | (Human) | | |
| | | | GVPSRFSGSGSGTEFTLTISSLQPEDFA | | | | | | |
| | | | TYYCQQLNSYPGYTFGQGTKLEIK | | | | | | |
| | COV2- | 1678 | NLMLTQPHSVSESPGKTVTISCTGSSG | IGHV4-59 | IGHJ4 | IGLV6-57 | IGU3 | 2687 | ARLRWLRGGI |
| | 2841 | | SIASNYVQWYQQRPGSAPTTVIYEDN | (Human) | (Human) | (Human) | (Human) | | DF |
| | | | QRPSGVPDRFSGSIDSSSNSASLTISGL | | | | | | |
| | | | KTEDEADYYCQSYDSNNQVFGGGTKL | | | | | | |
| | | | TVL | | | | | | |
| | COV2- | 1679 | NFMLTQPHSVSESPGKTVTISCTRSSG | IGHV1-46 | IGHJ4 | IGLV6-57 | IGU2 | 2688 | ARGYGFVPN |
| | 2842 | | SIASSYVQWYQQRPGSAPTTVIYEDN | (Human) | (Human) | (Human) | (Human) | | VLYYFDY |
| | | | QRPSGVPDRFSGSIDSSSNSASLTISGL | | | | | | |
| | | | KTEDEADYYCQSYDSSDVVFGGGTKLT | | | | | | |
| | | | VL | | | | | | |
| | COV2- | 1680 | SYVLTQPPSVSVAPGKTARITCGGNNI | IGHV3- | IGHJ6 | IGLV3-21 | IGU2 | 2689 | ARAQGGNYY |
| | 2844 | | GSKNVHWYQQKPGQAPVKVVYHDG | 303 | (Human) | (Human) | (Human) | | YGMDV |
| | | | DRPSGIPERFSGSNSGNTATLTINRVE | (Human) | | | | | |
| | | | AGDEADYSCQVWDSSSDHHVVFGGG | | | | | | |
| | | | TKLTVL | | | | | | |
| | COV2- | 1681 | SYELTQPPSVSVSPGQTARITCFGDALP | IGHV3-30 | IGHJ6 | IGLV3-25 | IGU3 | 2690 | ARDLAYHPYR |
| | 2848 | | KQYAYWYQQKPGQGPVLVIYKDSERP | (Human) | (Human) | (Human) | (Human) | | DYGDDDYYYY |
| | | | SGIPERFSGSTSGTTVTLTISGVQAEDE | | | | | | YGMDV |
| | | | ADYYCQSADSSGTYRVFGGGTKLTVL | | | | | | |
| | COV2- | 1682 | EIVMTQSPATLSVSPGERVTLSCRASQ | IGHV1-46 | IGHJ4 | IGKV3-15 | IGKJ4 | 2691 | ARGAIPPNSR |
| | 2853 | | SVSSNLAWCQQKPGQAPRLLIYGAST | (Human) | (Human) | (Human) | (Human) | | AEIDY |
| | | | RATGIPARFSGSGSGTEFTLTISSLQSE | | | | | | |
| | | | DFAVYYCQQYYNWPLTFGGGTKVEIK | | | | | | |
| | COV2- | 1683 | SYELTQPPSVSVSPGQTARITCSGDALP | IGHV1-46 | IGHJ6 | IGLV3-25 | IGU2 | 2692 | ARENDYGDY |
| | 2863 | | KQYAYWYQQKPGQAPVLVIYKDSERP | (Human) | (Human) | (Human) | (Human) | | VEPRDYYYG |
| | | | SGIPERFSGSSSGTTVTLTISGVQAEDE | | | | | | MDV |
| | | | ADYYCQSADSSAAYVVFGGGTKLTVL | | | | | | |
| | COV2- | 1684 | SYELTQPPSVSVSPGQTARITCSGDALP | IGHV3-33 | IGHJ3 | IGLV3-10 | IGU3 | 2693 | AREGPFGDRE |
| | 2872 | | KKYTYWYQQKSGQAPVVVIYEDSKRP | (Human) | (Human) | (Human) | (Human) | | ASGAFDV |
| | | | SGIPERFSGSSSGTMATLTISGAQVED | | | | | | |
| | | | EADYYCYSTDSSGKGVFGGGTKLTVL | | | | | | |
| | COV2- | 1685 | SYVLTQPPSVSVAPGKTARITCGGNNI | IGHV3-11 | IGHJ1 | IGLV3-21 | IGU1 | 2694 | TGVVAAPAEY |
| | 2873 | | GSKSVHWYQQKPGQAPVLVVYDDSD | (Human) | (Human) | (Human) | (Human) | | FQH |
| | | | RPSGIPERFSGSNSGNTATLTISRVEAG | | | | | | |
| | | | DEADYYCQVWDSSSDPFYVFGTGTKV | | | | | | |
| | | | TVL | | | | | | |
| | COV2- | 1686 | NFMLTQPHSVSESPGKTVTISCTGSSG | IGHV3-7 | IGHJ4 | IGLV6-57 | IGU3 | 2695 | ARLGFYYGGA |
| | 2878 | | SIASNYVQWYQQRPGSAPTTVISEDN | (Human) | (Human) | (Human) | (Human) | | DY |
| | | | QRPSGVPDRFSGSIDSSSNSASLTISGL | | | | | | |
| | | | KTEDEADYYCQSYDGINRAWVFGGGT | | | | | | |
| | | | KLTVL | | | | | | |
| | COV2- | 1687 | DIVMTQSPDSLAVSLGERATINCKSSQ | IGHV3-30 | IGHJ4 | IGKV4-1 | IGKJ1 | 2696 | AKDGSIAAAD |
| | 2883 | | SVLHSSNNKDSLVWYQQKPGQPPKLL | (Human) | (Human) | (Human) | (Human) | | Y |
| | | | IYWASSRESGVPDRFSGSGSGTDFTLTI | | | | | | |
| | | | SSLQAEDVAVYYCQQYYSTPWTFGQG | | | | | | |
| | | | TKVEIK | | | | | | |
| | COV2- | 1688 | DIQMTQSPSTLSASVGDRVTITCRASQ | IGHV1-46 | IGHJ4 | IGKV1-5 | IGKJ2 | 2697 | ARGAAVPAA |
| | 2891 | | SISSWLAWYQQKPGKAPKLLIYKASSL | (Human) | (Human) | (Human) | (Human) | | GEFDY |
| | | | ESGVPSRFSGSGSGTEFTLTISSLQPDD | | | | | | |
| | | | FATYYCQQYNSYSYTFGQGTKLEIK | | | | | | |
| | COV2- | 1689 | EIVMTQSPATLSVSPGERATLSCRASQ | IGHV3-30 | IGHJ4 | IGKV3-15 | IGKJ4 | 2698 | AKGDGSYLM |
| | 2894 | | SVSNNLAWYQQKPGQAPRLLIYGAST | (Human) | (Human) | (Human) | (Human) | | DYFDY |
| | | | RATGIPVRFSGSGSGTEFTLTISSLQSE | | | | | | |
| | | | DFAVYYCQQYDDWPPEVTFGG | | | | | | |
| | | | GTKVEIK | | | | | | |
| | COV2- | 1690 | QSVLTQPPSVSGAPGQRVTISCTGSSS | IGHV3-53 | IGHJ3 | IGLV1-40 | IGU2 | 2699 | ARSYDILTGYR |
| | 2901 | | NIGSGSDVHWYQQLPGTAPKLLIYGN | (Human) | (Human) | (Human) | (Human) | | DAFDI |
| | | | TNRPSGVPDRFSGSKSGTSASLAITGL | | | | | | |
| | | | QAEDEADYYCVAWDDSRNGLVFGGG | | | | | | |
| | | | AKLTVL | | | | | | |
| | COV2- | 1691 | QSVLTQPPSVSGVPGQRVTVSCTGSSS | IGHV4-34 | IGHJ6 | IGLV1-40 | IGU2 | 2700 | ARCRQMGNF |
| | 2904 | | NIGAGFDVYWYQQFLGTAPKLLIYGN | (Human) | (Human) | (Human) | (Human) | | YYYYMDV |
| | | | NNRPSGVPDRFSASKSGTSASLAITGL | | | | | | |
| | | | QAEDEADYYCQSFDIGRGGWIFGGGT | | | | | | |
| | | | KLTVL | | | | | | |
| | COV2- | 1692 | DIQMTQSPSSLSASVGDRVTITCRPSQ | IGHV5-51 | IGHJ6 | IGKV1-39 | IGKJ5 | 2701 | ARLGSESKIDY |
| | 2906 | | SITTYLNWYQQKPGKAPRLLIYAVSSLQ | (Human) | (Human) | (Human) | (Human) | | YYYGMDV |
| | | | SGVPSRFSGSGSGTDFTLTISSLQPEDF | | | | | | |
| | | | ATYYCQQSYSTPPTFGQGTRLEIK | | | | | | |
| | COV2- | 1693 | QSVLTQPPSVSGAPGQRVTISCTGSSS | IGHV1-69 | IGHJ6 | IGLV1-40 | IGU2 | 2702 | ARGRGYSNY |
| | 2909 | | NIGAGYDVHWYQQLPETAPKLLIYAN | (Human) | (Human) | (Human) | (Human) | | GASYYMDV |
| | | | SNRPSGVPDRFSGSKSGTSASLAITGL | | | | | | |
| | | | QAEDEADYYCQSYDSSLSGSVFGGGT | | | | | | |
| | | | KLTVL | | | | | | |
| | COV2- | 1694 | QSALTQPASVSGSPGQSITISCTGTSSD | IGHV4-39 | IGHJ3 | IGLV2-23 | IGU2 | 2703 | ARMSRGYNY |
| | 2911 | | VGSYNLVSWYQQHPGKAPKLKIYEGS | (Human) | (Human) | (Human) | (Human) | | AYTFDI |
| | | | KRPSGVSNRFSGSKSGNTASLTISGLQ | | | | | | |
| | | | AEDEADYYCCSYAGSVLFGGGTKLTVL | | | | | | |
| | COV2- | 1695 | DIQMTQSPSSLSASAGDRVTITCRASQ | IGHV2-70 | IGHJ3 | IGKV1-39 | IGKJ2 | 2704 | ARTMATINAF |
| | 2919 | | SIRYYLNWYQQKPGKAPKLLIYAASSL | (Human) | (Human) | (Human) | (Human) | | DI |
| | | | QSGVPSRFSGSGSGTDFTLTISSLQPED | | | | | | |
| | | | FAAYYCQQSFSTPRTFGQGTKLEIK | | | | | | |
| | COV2- | 1696 | SYVLTQPPSVSVAPGKTANITCGGNNI | IGHV3- | IGHJ4 | IGLV3-21 | IGU3 | 2705 | ARGGATNFD |
| | 2933 | | GRKSVHWYQQKSGQAPVLVVYDDSD | 303 | (Human) | (Human) | (Human) | | Y |
| | | | RPSGIPERFSGSNSGNTATLTISRVEAG | (Human) | | | | | |
| | | | DEADYYCQVWDSSSDHPEWVFGGGT | | | | | | |
| | | | KLTVL | | | | | | |
| | COV2- | 1697 | DIVMTQSPLSLPVTPGEPASISCRSSQS | IGHV7-4- | IGHJ4 | IGKV2-28 | IGKJ1 | 2706 | ARPGKAAAF |
| | 2934 | | LLHSNGYNFLDWYLQKPGQSPQLLIYL | 1 | (Human) | (Human) | (Human) | | DY |
| | | | GSNRASGVPDRFSGSGSGTDFTLKISR | (Human) | | | | | |
| | | | VQAEDVGVYYCMQALQTPWTFGQG | | | | | | |
| | | | TKVEIK | | | | | | |
| | COV2- | 1698 | SSELTQDPAVSVALGQTVRITCQGDSL | IGHV3-33 | IGHJ6 | IGLV3-19 | IGU3 | 2707 | ARDLHQDWV |
| | 2939 | | RRYYASWYQQKPGQAPVLVIYGKNN | (Human) | (Human) | (Human) | (Human) | | VVVAANVYG |
| | | | RPSGIPDRFSGSSSGNTASLTITGAQAE | | | | | | MDV |
| | | | DEADYYCNSRDSSGNPRWFGGGTKLT | | | | | | |
| | | | VL | | | | | | |
| | COV2- | 1699 | EIVLTQSPGTLSLSPGERATLSCRASQS | IGHV1-58 | IGHJ3 | IGKV3-20 | IGKJ2 | 2708 | AAPYCSSISCN |
| | 2941 | | VSSSYLAWYQQKPGQAPRLLIYGASSR | (Human) | (Human) | (Human) | (Human) | | DGFDI |
| | | | ATGIPDRFSGSGSGTDFTLTISRLEPED | | | | | | |
| | | | FAVYYCQQYGSSPPRYTFGQGTKLEIK | | | | | | |
| | COV2- | 1700 | SYELTQPPSVSVSPGQTARITCSTDALP | IGHV2-70 | IGHJ6 | IGLV3-25 | IGU3 | 2709 | ARATTFFYGM |
| | 2942 | | NEYIYWYQQKPGQAPVLVIYKDSERPS | (Human) | (Human) | (Human) | (Human) | | DV |
| | | | GIPERFSGSSSGTTVTLTISGVQAEDEA | | | | | | |
| | | | DYYCQSVDSSGTYRVFGGGTKLTVL | | | | | | |
| | COV2- | 1701 | DIQMTQSPSSLSASVGDRVTITCQASQ | IGHV3-30 | IGHJ4 | IGKV1-33 | IGKJ3 | 2710 | AKKGGPYCG |
| | 2944 | | DISNYLNWYQQKAGKAPKLLIYDASNL | (Human) | (Human) | (Human) | (Human) | | GGNCYAGYF |
| | | | ETGVPSRFSGSGSGTDFTFTISSLQPDD | | | | | | DY |
| | | | IATYYCQQYDNLPLAFGPGTKVDVK | | | | | | |
| | COV2- | 1702 | DVVMTQSPLSLPVTLGQPASISCRSSQ | IGHV3-9 | IGHJ3 | IGKV2-30 | IGKJ2 | 2711 | AKLDVGGYDF |
| | 2945 | | SLVYSDGNTYLNWFQQRPGQSPRRLI | (Human) | (Human) | (Human) | (Human) | | VSGHYYAFDI |
| | | | YKVSSRDSGVPDRFSGSGSGTAFTLKIS | | | | | | |
| | | | RVEAEDVGVYYCMQGTHWPYTFGQ | | | | | | |
| | | | GTKLEIK | | | | | | |
| | COV2- | 1703 | QSALTQPRSVSGSPGQSVTISCTGTSS | IGHV7-4- | IGHJ6 | IGLV2-11 | IGU3 | 2712 | ARGLISLFRGA |
| | 2947 | | DVGGYTYVSWYQQHPGKAPKLMIYD | 1 | (Human) | (Human) | (Human) | | IFHYYYGMDV |
| | | | VNKRPSGVPDRFSGSKSGITASLTISGL | (Human) | | | | | |
| | | | QAEDEADYYCCSYAGRYTWVFGGGTT | | | | | | |
| | | | LTVL | | | | | | |
| | COV2- | 1704 | DIQLTQSPSFLSASVGDRVTITCRASQG | IGHV3-66 | IGHJ6 | IGKV1-9 | IGKJ5 | 2713 | ARDLVTYGLD |
| | 2952 | | ISNYLAWYQQKPGTAPNLLIYAASTLQ | (Human) | (Human) | (Human) | (Human) | | V |
| | | | SGVPSRFSGSGSGTEFTLTISSLQPEDF | | | | | | |
| | | | ATYYCQLLNSHPLTFGQGTRLEIK | | | | | | |
| | COV2- | 1705 | SYELTQPPSVSVSPGQTARITCSGDALP | IGHV2-5 | IGHJ5 | IGLV3-25 | IGU3 | 2714 | AHSGPPDLSP |
| | 2953 | | KQYAYWYQQKPGQAPVLVIYRDSERP | (Human) | (Human) | (Human) | (Human) | | VLSQGWFDP |
| | | | SGIPERFSGSTSGTTVTLTISGVQAEDE | | | | | | |
| | | | ADYYCQSADSTGWVFGGGTKLTVL | | | | | | |
| | COV2- | 1706 | DIVMTQTPLSLSVTPGQPASFSCKSSQ | IGHV3-30 | IGHJ6 | IGKV2D-29 | IGKJ2 | 2715 | AKGGPNKEVL |
| | 2955 | | SLLHSDGKTYLYWYLQKPGQSPQLLIY | (Human) | (Human) | (Human) | (Human) | | YFGELLDYGM |
| | | | AVSNRFSGVPDRFSGSGSGTDFTLKIS | | | | | | DV |
| | | | RVEAEDVGVYYCMQSIQLPYTFGQGT | | | | | | |
| | | | KLEIK | | | | | | |
| | COV2- | 1707 | DIVVTQTPLSLSVTPGQPASISCKSSET | IGHV1-18 | IGHJ4 | IGKV2D-29 | IGKJ5 | 2716 | ARVQRRRLDY |
| | 2960 | | LLHSDGKTYLSWYLQKPGQPPQLLIYEV | (Human) | (Human) | (Human) | (Human) | | |
| | | | SNRFSGVPDRFSGSGSGTDFTLKIGRV | | | | | | |
| | | | EAEDVGLYYCMQSIQLAFGQGTRLEIE | | | | | | |
| | COV2- | 1708 | ETVLTQSPGTLSLSPGERATLSCRASQS | IGHV1-58 | IGHJ3 | IGKV3-20 | IGKJ1 | 2717 | AAPYCSSISCN |
| | 2961 | | VSSSYLAWYQQKPGQAPRLLIYGASSR | (Human) | (Human) | (Human) | (Human) | | DGFDI |
| | | | ATGIPDRFSGSGSGTDFTLTISRLEPED | | | | | | |
| | | | FAVYYCQHYGSSRGWTFGQGTKVEIK | | | | | | |
| | COV2- | 1709 | EIVMTQSPATLSVSPGERVTLSCRASQ | IGHV1-46 | IGHJ4 | IGKV3-15 | IGKJ4 | 2718 | ARGAIPPNSR |
| | 2997 | | SVSSNLAWCQQKPGQAPRLLIYGAST | (Human) | (Human) | (Human) | (Human) | | AEIDY |
| | | | RATGIPARFSGSGSGTEFTLTISSLQSE | | | | | | |
| | | | DFAVYYCQQYYNWPLTFGGGTKVEIK | | | | | | |
| | COV2- | 1710 | DIQMTQSPSTLSASIGDRVTITCRASQS | IGHV3-30 | IGHJ3 | IGKV1-5 | IGKJ2 | 2719 | ARDLSTTWYL |
| | 3010 | | ISSWLAWYQQIPGKAPKLLIYKASSLES | 3 | (Human) | (Human) | (Human) | | EMWGPDAF |
| | | | GVPSRFSGSGSGTEFTLTISSLQPDDFA | (Human) | | | | | DI |
| | | | TYYCQQYNSYPYTFGQGTKLEIK | | | | | | |
| | COV2- | 1711 | DIQMTQSPSSLSASVGDRVTITCRASQ | IGHV3-13 | IGHJ5 | IGKV1-39 | IGKJ1 | 2720 | ARVLYDSSGF |
| | 3012 | | SISSYLNWYQQKPGKAPKLLIYAASSLE | (Human) | (Human) | (Human) | (Human) | | YNWFDP |
| | | | SGVPSRFSGSGSGTDFTLTISSLQPEDF | | | | | | |
| | | | ATYYCQQSYEIPPWTFGQGTKVEIK | | | | | | |
| | COV2- | 1712 | DIQMTQSPSSLSASVGDRVTITCQASQ | IGHV3-30 | IGHJ4 | IGKV1-33 | IGKJ3 | 2721 | AKNLGPYCSG |
| | 3013 | | DISNYLNWYQQKPGKAPKLLIYDASNL | (Human) | (Human) | (Human) | (Human) | | GTCYSLVGDY |
| | | | ETGVPSRFSGSGSGTDFTFTISSLQPED | | | | | | |
| | | | IATYYCQQYANLPFTFGPGTKVDIK | | | | | | |
| | COV2- | 1713 | EIVLTQSPGTLSLSPGERATLSCRASQS | IGHV1-58 | IGHJ3 | IGKV3-20 | IGKJ1 | 2722 | AAPYCSSISCN |
| | 3025 | | VSSSYLAWYQQKPGQAPRLLIYGASSR | (Human) | (Human) | (Human) | (Human) | | DGFDI |
| | | | ATGIPDRFSGSGSGTDFTLTISRLEPED | | | | | | |
| | | | FAVYYCQHYGSSRGWTFGQGTKVEIK | | | | | | |
| | COV2- | 1714 | SYELTQPPSVSVSPGQTARITCSTDALP | IGHV3-43 | IGHJ6 | IGLV3-25 | IGU2 | 2723 | AKDEMAYPP |
| | 3029 | | NEYIYWYQQKPGQAPVLVIYKDSERPS | (Human) | (Human) | (Human) | (Human) | | SHHYYYYYMD |
| | | | GIPERFSGSSSGTTVTLTISGVQAEDEA | | | | | | V |
| | | | DYYCQSVDSSGTYPHVIFGGGTKLTVL | | | | | | |
| | COV2- | 1715 | QSVLTQPPSVSGAPGQRVTISCTGSSS | IGHV3-7 | IGHJ6 | IGLV1-40 | IGU2 | 2724 | ARLVTTVTTA |
| | 3057 | | NIGAGYDVHWYQQLPGTAPKLLIYGN | (Human) | (Human) | (Human) | (Human) | | NGLYYYSYYY |
| | | | TNRPSGVPDRFSGSKSGTSASLAITGL | | | | | | MDV |
| | | | QAEDEADYYCQSYASSLSAHVVFGGG | | | | | | |
| | | | TKLTVL | | | | | | |
| | COV2- | 1716 | DIVMTQSPDSLAVSLGERATINCKSSQ | IGHV3-30 | IGHJ3 | IGKV4-1 | IGKJ2 | 2725 | ARDRSGNYR |
| | 3058 | | SVLYSSNNENYLAWYQQKPGQPPKLLI | (Human) | (Human) | (Human) | (Human) | | DAFDI |
| | | | YWASTRESGVPDRFSGSGSGTDFTLTI | | | | | | |
| | | | SSLQAEDVAVYYCQQYYSSYTFGQGTK | | | | | | |
| | | | VEIK | | | | | | |
| | COVA1- | 1717 | EIVMTQSPGTLSLSPGERATLSCRASQ | IGHV4-39 | IGHJ4 | IGKV3-20 | IGKJ3 | 2726 | ARVSSGYYFT |
| | 01 | | SVSSSYLAWYQQKPGQAPRLLIYGASS | (Human) | (Human) | (Human) | (Human) | | PFDY |
| | | | RATGIPDRFSGSGSGTDFTLTISRLEPE | | | | | | |
| | | | DFAVYYCQQYGSSPPPFTFGPGTKVDI | | | | | | |
| | | | K | | | | | | |
| | COVA1- | 1718 | NFMLTQPASVSGSPGQSITISCTGASS | IGHV3-30 | IGHJ3 | IGLV2-14 | IGU3 | 2727 | ARARGGSYN |
| | 02 | | DVGGYNYVSWYQQHPGKAPKLMIYD | (Human) | (Human) | (Human) | (Human) | | DAFDI |
| | | | VSNRPSGVSNRFSGSKSGNTASLTISGL | | | | | | |
| | | | QAEDEADYYCSSYTSSSTPVVFGGGTE | | | | | | |
| | | | LTVL | | | | | | |
| | COVA1- | 1719 | DIVMTQSPSSLSASVGDRVTITCRASQ | IGHV3-30 | IGHJ6 | IGKV1-27 | IGKJ3 | 2728 | AKDGQYYDF |
| | 03 | | GISNYLAWYQQKPGKVPKLLIYAASTL | (Human) | (Human) | (Human) | (Human) | | WSGYLGART |
| | | | QSGVPSRFSGSGSGTDFTLTISSLQPED | | | | | | NPHYYYYMD |
| | | | VATYYCQKYNSAPPAFGQGTKVDIK | | | | | | V |
| | COVA1- | 1720 | QSALTQPPSVSVSPGQTASIPCSGDKL | IGHV3-9 | IGHJ6 | IGLV3-1 | IGU3 | 2729 | AKDMGEAVA |
| | 06 | | GDIYACWYQQKPGQSPVLVIYQDTKR | (Human) | (Human) | (Human) | (Human) | | GTHYGMDV |
| | | | PSGIPERFSGSNSGNTATLTISGTOAM | | | | | | |
| | | | DEADYYCQAWGSTTAKVFGGGTKLTV | | | | | | |
| | | | P | | | | | | |
| | COVA1- | 1721 | DIQLTQSPATLSLSPGERATLSCRASQS | IGHV1-69 | IGHJ4 | IGKV3-11 | IGKJ4 | 2730 | ARVGAYDSSG |
| | 07 | | VSSYLAWYQQKPGQAPRLLIYDASNR | (Human) | (Human) | (Human) | (Human) | | YSNDY |
| | | | ATGIPARFSGSGSGTDFTLTISSLEPED | | | | | | |
| | | | FAVYYCQQRSNWPPRVTFGGGTKVEIK | | | | | | |
| | COVA1- | 1722 | QSVLTQPASVSGSPGQSITISCTGTSSD | IGHV3-30 | IGHJ4 | IGLV2-14 | IGU3 | 2731 | AREDYYDSSG |
| | 08 | | VGGYNYVSWYQQHPGKAPKLMIYDV | (Human) | (Human) | (Human) | (Human) | | SFDY |
| | | | SNRPSGVSNRFSGSKSGNTASLTISGL | | | | | | |
| | | | QAEDEADYYCSSYTSSSTRHWVFGGG | | | | | | |
| | | | TKLTVL | | | | | | |
| | COVA1- | 1723 | DIQMTQSPSSLSASVRDRVTITCRASQ | IGHV4-59 | IGHJ3 | IGKV1-39 | IGKJ1 | 2732 | ARHSQGWLQ |
| | 09 | | SITSSLNWYQQKPGKAPKLLIYAASSLQ | (Human) | (Human) | (Human) | (Human) | | QAVAFDI |
| | | | SGVPSRFSGSGSGTDFTLTISSLQPEDF | | | | | | |
| | | | ATYYCQQSYSTPYTFGQGTKVEIK | | | | | | |
| | COVA1- | 1724 | NFMLTQPASVSGSPGQSITISCTGSSS | IGHV3-66 | IGHJ4 | IGLV2-14 | IGU3 | 2733 | ARGGYYYDPS |
| | 10 | | DIAPYTFVSWYQQHSGTAPKLIIYDVR | (Human) | (Human) | (Human) | (Human) | | GYYSRSFSFDY |
| | | | NRPSGISDRFSGSRSGNTASLSISGLQA | | | | | | |
| | | | EDEADYYCSAYTTTSTSWVFGGGTKLT | | | | | | |
| | | | VL | | | | | | |
| | COVA1- | 1725 | QSALTQPPSASGSPGQSVTISCTGTSS | IGHV1-2 | IGHJ6 | IGLV2-8 | IGU3 | 2734 | ARDLVWATV |
| | 12 | | DIGTYNYVSWYQQHPGKAPKLMIYEV | (Human) | (Human) | (Human) | (Human) | | SGTMDV |
| | | | TKRPSGVPDRFSGSKSGNTASLTVSGL | | | | | | |
| | | | QADDEGDYYCSSYVGNNNWVFGGG | | | | | | |
| | | | TKLTVL | | | | | | |
| | COVA1- | 1726 | DIQLTQSPSSLSASVGDRVTITCQASQ | IGHV1-46 | IGHJ1 | IGKV1-33 | IGKJ4 | 2735 | ARPPRNYYDR |
| | 16 | | DISNYLNWYQQRPGKAPKLLIYDASNL | (Human) | (Human) | (Human) | (Human) | | SGYYQRAEYF |
| | | | ETGVPSRFSGSGSGTDFTFTISSLQPED | | | | | | QH |
| | | | IATYYCQQYDNPPLTFGGGTKLEIK | | | | | | |
| | COVA1- | 1727 | QSALTQEPSLTVSPGGTVTLTCGSSTG | IGHV3-66 | IGHJ4 | IGLV7-46 | IGU3 | 2736 | ARVEWAAAG |
| | 18 | | AVTSGHYPYWFQQKPGQAPRTLIYDT | (Human) | (Human) | (Human) | (Human) | | TFY |
| | | | SNKRSWTPARFSGSLLGGKAALTLSGA | | | | | | |
| | | | QPEDEAEYYCLLSYSGVWVFGGGTKL | | | | | | |
| | | | TVL | | | | | | |
| | COVA1- | 1728 | EIVMTQSPATLSVSPGERATLSCRASQ | IGHV3-21 | IGHJ3 | IGKV3-15 | IGKJ1 | 2737 | ARWKSDYYD |
| | 19 | | SVSSNLAWYQQKPGQAPRLLIYGAST | (Human) | (Human) | (Human) | (Human) | | SSGYYPAAFDI |
| | | | RATGIPARFSGSGSGTEFTLTISSLQSE | | | | | | |
| | | | DFAVYYCQQYNNWPPWTFGQGTKV | | | | | | |
| | | | EIK | | | | | | |
| | COVA1- | 1729 | NFMLTQPPSVSVAPGKTARITCGGNN | IGHV3-21 | IGHJ6 | IGLV3-21 | IGU3 | 2738 | AGDQNLYCS |
| | 20 | | IGSKSVHWYQQKPGQAPVLVIYYDSD | (Human) | (Human) | (Human) | (Human) | | GDSCYYHYYG |
| | | | RPSGIPERFSGSNSGNTATLTISRVEAG | | | | | | MDV |
| | | | DEADYYCQVWDSSSDHWVFGGGTKL | | | | | | |
| | | | TVL | | | | | | |
| | COVA1- | 1730 | EIVMTQSPATLSVSPGERATLSYRASQ | IGHV3-30 | IGHJ6 | IGKV3-15 | IGKJ2 | 2739 | ARDSEYYDILT |
| | 21 | | SVSSNLAWYQQKPGQAPRLLIYGAST | (Human) | (Human) | (Human) | (Human) | | GYLAPTHYYY |
| | | | RATGIPARFSGSGSGTEFTLTISSLQSE | | | | | | YYMDV |
| | | | DFAVYYCQQYNNWPPGTFGQGTKLEI | | | | | | |
| | | | K | | | | | | |
| | COVA1- | 1731 | SYELTQPPSVSVSPGQTASITCSGDKLG | IGHV1-18 | IGHJ6 | IGLV3-1 | IGU3 | 2740 | ARDLVDTAM |
| | 22 | | DKYACWYQQKPGQSPVLVTYQDNKR | (Human) | (Human) | (Human) | (Human) | | VQTLDDYGM |
| | | | PSGIPERFSGSNSGNTATLTISGTQAM | | | | | | DV |
| | | | DEADYYCQAWDSSTAVFGGGTKLTVL | | | | | | |
| | COVA1- | 1732 | SYELTQPPSVSVSPGQTARITCSGDALP | IGHV5-51 | IGHJ4 | IGLV3-25 | IGU3 | 2741 | ARYYYDSRGY |
| | 23 | | KQYAYWYQQKPGQAPVLVIYKDSERP | (Human) | (Human) | (Human) | (Human) | | TSIDF |
| | | | SGIPERFSGSSSGTTVTLTISGVQAEDE | | | | | | |
| | | | ADYYCQSADSSGTYSVVFGGGTKLTVL | | | | | | |
| | COVA1- | 1733 | DIQLTQSPSTLSASVGDRVTITCRASQS | IGHV4-39 | IGHJ6 | IGKV1-5 | IGKJ3 | 2742 | ARLNYDFWS |
| | 25 | | ISSWLAWYQQKPGKAPKLLIYKASSLE | (Human) | (Human) | (Human) | (Human) | | GYYSYALYYM |
| | | | SGVPSRFSGSGSGTEFTLTISSLQPDDF | | | | | | DV |
| | | | ATYYCQQYNSYSITFGPGTKVDIK | | | | | | |
| | COVA1- | 1734 | DIVMTQSPSFLSASVGDRVTITCRASQ | IGHV4-31 | IGHJ4 | IGKV1-9 | IGKJ4 | 2743 | ARQQLDYYDS |
| | 26 | | GISSYLAWYQQKPGKAPKLLIYAASTL | (Human) | (Human) | (Human) | (Human) | | SGCFDY |
| | | | QSGVPSRFSGSGSGTEFTLTISSLQPED | | | | | | |
| | | | FATYYCQQLHSYPLTFGGGTKVDIK | | | | | | |
| | COVA1- | 1735 | DVVMTQSPGTLSLSPGERATLSCRAS | IGHV4-59 | IGHJ4 | IGKV3-20 | IGKJ3 | 2744 | ARAMGSYRS |
| | 27 | | QNVSSSYLAWYQQKPGQAPRLLIYGA | (Human) | (Human) | (Human) | (Human) | | PFDY |
| | | | SSRATGIPDRFSGSGSGTDFTLTISRLE | | | | | | |
| | | | PEDFAVYYCQQYGSSPLFTFGPGTKVEI | | | | | | |
| | | | K | | | | | | |
| | COVA2- | 1736 | DIVMTQSPSSLSASVGDRVTITCRASQ | IGHV3-13 | IGHJ2 | IGKV1-39 | IGKJ4 | 2745 | ARGGDRYPV |
| | 01 | | SISSYLNWYQQKPGKAPKLLIYAASSLQ | (Human) | (Human) | (Human) | (Human) | | GYFDL |
| | | | SGVPSRFSGSGSGTDFTLTISSLQPEDF | | | | | | |
| | | | ATYYCQQSYSTPPVTFGGGTKVEIK | | | | | | |
| | COVA2- | 1737 | DIVMTQSPSSLSASVGDRVTITCRASQ | IGHV4-39 | IGHJ6 | IGKV1-39 | IGKJ3 | 2746 | ARRSTSRWG |
| | 02 | | SISNYLNWYQQKPGKAPKLLLYAASDL | (Human) | (Human) | (Human) | (Human) | | YYYMDV |
| | | | QSGVPSRFSGSGSGTDFTLTISSLQPED | | | | | | |
| | | | FATYYCQQSYSTHMSTFGQGTKVDIK | | | | | | |
| | COVA2- | 1738 | EIVMTQSPATLSLSPGERATLSCRASQS | IGHV3-48 | IGHJ4 | IGKV3-11 | IGKJ5 | 2747 | AREANSDFW |
| | 03 | | VSSYLAWYQQKPGQAPRLLIYDASNR | (Human) | (Human) | (Human) | (Human) | | SGYLGYFDY |
| | | | ATGIPARFSGSGSGTDFTLTISSLEPED | | | | | | |
| | | | FAVYYCQQRSNWPQVTFGQGTRLEIK | | | | | | |
| | COVA2- | 1739 | EIVMTQSPGTLSLSPGERATLSCRASQ | IGHV3-53 | IGHJ6 | IGKV3-20 | IGKJ3 | 2748 | ARDLERAGG |
| | 04 | | SVSSSYLAWYQQKPGQAPRLLIYGASS | (Human) | (Human) | (Human) | (Human) | | MDV |
| | | | RATGIPDRFSGSGSGTDFTLTISRLEPE | | | | | | |
| | | | DFAVYYCQQYGSLYTFGQGTKVDIK | | | | | | |
| | COVA2- | 1740 | DIQMTQSPSSLSASVGDRVTITCQASQ | IGHV5-51 | IGHJ6 | IGKV1-33 | IGKJ4 | 2749 | ARHMRPSIAA |
| | 05 | | DISNYLNWYQQKPGKAPKLLIYDASNL | (Human) | (Human) | (Human) | (Human) | | RPGYQYYMD |
| | | | ETGVPSRFSGSGSGTDFTFTISSLQPED | | | | | | V |
| | | | IATYYCQQYDNLPLTFGGGTKLEIK | | | | | | |
| | COVA2- | 1741 | EIVLTQSPGTLSLSPGERATLSCRASQS | IGHV3-53 | IGHJ6 | IGKV3-20 | IGKJ3 | 2750 | AREAYGMDV |
| | 07 | | VSSSYLAWYQQKPGQAPRLLIYGASSR | (Human) | (Human) | (Human) | (Human) | | |
| | | | ATGIPDRFSGSWSGTDFTLTISRLEPED | | | | | | |
| | | | FAVYYCQQYGSSPGTFGQGTKVDIK | | | | | | |
| | COVA2- | 1742 | DIVMTQSPGTLSLSPGERATLSCRASQ | IGHV3-23 | IGHJ1 | IGKV3-20 | IGKJ4 | 2751 | AKGLRGQQL |
| | 10 | | SVSSSYLAWYQQKPGQAPRLLIYGASS | (Human) | (Human) | (Human) | (Human) | | VIPTEYFQH |
| | | | RATGIPDRFSGSGSGTDFTLTISRLEPE | | | | | | |
| | | | DFAVYYCQQYGSSLLTFGGGTKVEIK | | | | | | |
| | COVA2- | 1743 | DIQLTQSPGTLSLSPGERATLSCRASQS | IGHV1-69 | IGHJ4 | IGKV3-20 | IGKJ4 | 2752 | ARGPRGCSST |
| | 11 | | VSSSYLAWYQQKPGQAPRLLIYGASSR | (Human) | (Human) | (Human) | (Human) | | SCYGSYFDY |
| | | | ATGIPDRFSGSGSGTDFTLTISRLEPED | | | | | | |
| | | | FAVYYCQQYGSSPRLTFGGGTKVDIK | | | | | | |
| | COVA2- | 1744 | EIVLTQSPATLSVSPGERATLSCRASQS | IGHV3-21 | IGHJ4 | IGKV3-15 | IGKJ1 | 2753 | ARDQPLPDIL |
| | 12 | | VSSSLAWYQQKPGQAPRLLIYAASTRA | (Human) | (Human) | (Human) | (Human) | | TGYYTGPLDY |
| | | | TGIPARFSGSGSGTEFTLTISSLQSEDF | | | | | | |
| | | | AVYYCQQYNNWPPWTFGQGTKLEIK | | | | | | |
| | COVA2- | 1745 | EIVMTQSPGTLSLSPGERATLSCRASQ | IGHV3-53 | IGHJ6 | IGKV3-20 | IGKJ1 | 2754 | ARDLDTMGG |
| | 13 | | SVSSSYLAWYQQKPGQAPRLLIYGASS | (Human) | (Human) | (Human) | (Human) | | MDV |
| | | | RATGIPDRFSGSGSGTDFTLTISRLEPE | | | | | | |
| | | | DFAVYYCQQYGSSPGTFGQGTKVEIK | | | | | | |
| | COVA2- | 1746 | EIVLTQSPATLSLSPGERATLSCRASQS | IGHV1-69 | IGHJ4 | IGKV3-11 | IGKJ1 | 2755 | ARVRYYDSSG |
| | 14 | | VSSYLAWYQQEPGQAPRLLIYDASNR | (Human) | (Human) | (Human) | (Human) | | YYEDY |
| | | | ATGIPARFSGSGSGTDFTLTISSLEPED | | | | | | |
| | | | FAVYYCQQRSNWPPMYTFGQGTKVEI | | | | | | |
| | | | K | | | | | | |
| | COVA2- | 1747 | DIVMTQSPLSLPVTLGQPASISCRSSQS | IGHV3-23 | IGHJ4 | IGKV2-30 | IGKJ2 | 2756 | AKDTGYCGD |
| | 15 | | LVYSDGNTFLNWFQQRPGQSPRRLIY | (Human) | (Human) | (Human) | (Human) | | DCYIKLIRGGP |
| | | | QVSNRDSGVPDRFSGSGSGTDFTLKIS | | | | | | DY |
| | | | RVEAEDVGVYYCMQGTHWPRTFGQ | | | | | | |
| | | | GTKLEIK | | | | | | |
| | COVA2- | 1748 | DIQLTQSPSSLSASVGDRVTITCRASQG | IGHV1-69 | IGHJ4 | IGKV1-17 | IGKJ1 | 2757 | ASSDSSGFVG |
| | 16 | | IRNDLGWYQQKPGKAPKRLIYAASSL | (Human) | (Human) | (Human) | (Human) | | SRGFDY |
| | | | QSGVPSRFSGSGSGTEFTLTISSLQPED | | | | | | |
| | | | FATYYCLQHNSYPPLFGQGTKVEIK | | | | | | |
| | COVA2- | 1749 | EIVMTQSPATLSLSPGERATLSCRASQS | IGHV1-69 | IGHJ4 | IGKV3-11 | IGKJ1 | 2758 | ASFGDDSGDE |
| | 17 | | VSSYLAWYQQKPGQAPRLLIYDASNR | (Human) | (Human) | (Human) | (Human) | | GVR |
| | | | ATGIPARFSGSGSGTDFTLTISSLEPED | | | | | | |
| | | | FAVYYCQQRSNWPPYTFGQGTKVEIK | | | | | | |
| | COVA2- | 1750 | EIVLTQSPATLSLSPGERATLSCRASQS | IGHV1-69 | IGHJ4 | IGKV3-11 | IGKJ5 | 2759 | ARVYSYDSSG |
| | 18 | | VSSYLAWYQQKPGQAPRLLIYDASNR | (Human) | (Human) | (Human) | (Human) | | YYLEY |
| | | | ATGIPARFSGSGSGTDFTLTISSLEPED | | | | | | |
| | | | FAVYYCQQRSNWPPSITFGQGTRLEIK | | | | | | |
| | COVA2- | 1751 | DVVMTQSPSSLSVSVGDRVTITCRASQ | IGHV3-53 | IGHJ6 | IGKV1-17 | IGKJ1 | 2760 | ASPLLLTPPDY |
| | 20 | | GIRNDLGWYQQKPGKAPKRLIYAASSL | (Human) | (Human) | (Human) | (Human) | | YYYMDV |
| | | | QSGVPSRFSGSGSGTEFTLTISSLQPED | | | | | | |
| | | | FATYYCLQHNSYLWTFGQGTKLEIK | | | | | | |
| | COVA2- | 1752 | AIRMTQSPSSLSASVGDRVTITCQASQ | IGHV4-4 | IGHJ4 | IGKV1-33 | IGKJ5 | 2761 | ARGPRYCSST |
| | 22 | | DISNYLNWYQQKPGKAPKLLIYDASNL | (Human) | (Human) | (Human) | (Human) | | SCYAGVYFDY |
| | | | ETGVPSRFSGSGSGTDFTFTISSLQPED | | | | | | |
| | | | IATYYCQQYDNLPITFGQGTRLEIK | | | | | | |
| | COVA2- | 1753 | EIVLTQSPGTLSLSPGERATLSCRASQS | IGHV1-69 | IGHJ4 | IGKV3-20 | IGKJ4 | 2762 | ARDGFGDVE |
| | 23 | | VSSSYLAWYQQKPGQAPRLLIYGASSR | (Human) | (Human) | (Human) | (Human) | | EMATIKDAFD |
| | | | ATGIPDRFSGSGSGTDFTLTISRLEPED | | | | | | |
| | | | FAVYYCQQYGSSPGVTFGGGTKLEIK | | | | | | |
| | COVA2- | 1754 | AIRMTQSPSSLSASVGDRVTITCRASQ | IGHV5-101 | IGHJ3 | IGKV1-39 | IGKJ2 | 2763 | ARPNPAGGY |
| | 24 | | SISSYLNWYQQKPGKAPKLLIYAASSLQ | (Human) | (Human) | (Human) | (Human) | | DSSGWVDAF |
| | | | SGVPSRFSGSGSGTDFTLTISSLQPEDF | | | | | | DI |
| | | | ATYYCQQSYSTPQTFGQGTKLEIK | | | | | | |
| | COVA2- | 1755 | QPVLTQPASVSGSPGQSITISCTGTSSD | IGHV1-24 | IGHJ5 | IGLV2-14 | IGU3 | 2764 | ATGPTIAAAA |
| | 25 | | VGGYNYVSWYQQHPGKAPKLMIYDV | (Human) | (Human) | (Human) | (Human) | | TNWFDP |
| | | | SNRPSGVSNRFSGSRSGDTASLTISGL | | | | | | |
| | | | QAEDEADYYCSSYTSSSTWVFGGGTKL | | | | | | |
| | | | TVL | | | | | | |
| | COVA2- | 1756 | QLVLTQPPSVSVSPGQTASITCSGDKL | IGHV3-15 | IGHJ6 | IGLV3-1 | IGU3 | 2765 | TTDRGDSYGY |
| | 26 | | GDKYACWYQQKPGQSPVLVIYQDSKR | (Human) | (Human) | (Human) | (Human) | | YYCMDV |
| | | | PSGIPERFSGSNSGNTATLTISGTQAM | | | | | | |
| | | | DEADYYCQAWDSSTAVVFGGGTKLTV | | | | | | |
| | | | L | | | | | | |
| | COVA2- | 1757 | EIVMTQSPATLSVSPGERATLSCRASQ | IGHV3-33 | IGHJ4 | IGKV3-15 | IGKJ5 | 2766 | AKDKAPPCSS |
| | 28 | | SVSSNLAWYQHKPGQAPRLLIYGASTR | (Human) | (Human) | (Human) | (Human) | | GWYYFDY |
| | | | ATGIPARFSGSGSGTEFTLTISSLQSED | | | | | | |
| | | | FAVYYCQQYNYWPLITFGQGTRLEIK | | | | | | |
| | COVA2- | 1758 | DIVMTQSPSSLSASVGDRVTITCRASQ | IGHV3-30 | IGHJ3 | IGKV1-39 | IGKJ1 | 2767 | ARGVEDPVV |
| | 29 | | SISSYLNWYQQKPGKAPKLLIYAASSLQ | (Human) | (Human) | (Human) | (Human) | | PAAIPWCWF |
| | | | SGVPSRFSGSGSGTDFTLTISSLQPEDF | | | | | | DP |
| | | | ATYYCQQSYSTPRTFGQGTKVEIK | | | | | | |
| | COVA2- | 1759 | QSVLTQPPSVSVAPGQTARITCGGNNI | IGHV4-30 | IGHJ5 | IGLV3-21 | IGU3 | 2768 | ASLPVVPAAI |
| | 30 | | GSKSVHWYQQKPGQAPVLVVYDDSD | (Human) | (Human) | (Human) | (Human) | | GPLPAFDI |
| | | | RPSGIPERFSGSNSGNTATLTISRVEAG | | | | | | |
| | | | DEADYYCQVWASSSVVFGGGTKLTVL | | | | | | |
| | COVA2- | 1760 | DIVMTQTPLSLSVTPGQPASISCKSGQ | IGHV1-2 | IGHJ3 | IGKV2D-29 | IGKJ1 | 2769 | RKMLTIFGKV |
| | 31 | | SLLHSDGKTYLYWYLQKPGQPPQLLIY | (Human) | (Human) | (Human) | (Human) | | NQTMLLISGA |
| | | | EVSNRFSGVPDRFSGSGSGTDFTLKISR | | | | | | KGQ |
| | | | VEAEDVGVYYCMQSIQLPPTFGQGTK | | | | | | |
| | | | VEIK | | | | | | |
| | COVA2- | 1761 | DIVMTQSPATLSLSPGERATLSCRASQ | IGHV1-69 | IGHJ4 | IGKV3-11 | IGKJ4 | 2770 | ARTHSYDNSG |
| | 32 | | SVSSFLAWYQQKPGQAPRLLIYDASNR | (Human) | (Human) | (Human) | (Human) | | QYFDY |
| | | | ATGIPARFSGSGSGTDFTLTISSLEPED | | | | | | |
| | | | FAVYYCQQRSNWPPRLTFGGGTKVDIK | | | | | | |
| | COVA2- | 1762 | QSALTQPASVSGSPGQSITISCTGTSSD | IGHV5-101 | IGHJ6 | IGLV2-23 | IGU3 | 2771 | ARLKVITIFGV |
| | 33 | | VGSYNLVSWYQQHPGKAPKLMIYDSS | (Human) | (Human) | (Human) | (Human) | | VRDDYGMDV |
| | | | KRPSGVSNRFSGSKSGNTASLTISGLQ | | | | | | |
| | | | AEDEADYYCCSYAGSVFGGGTKLTVL | | | | | | |
| | COVA2- | 1763 | QTVVTQPASVSGSPGQSITISCTGTSSD | IGHV3-30 | IGHJ4 | IGLV2-14 | IGU1 | 2772 | ARSASGSYYG |
| | 34 | | VGGYNYVSWYQQHPGKAPKLMIYDV | (Human) | (Human) | (Human) | (Human) | | AFDY |
| | | | SNRPSGVSNRFSGSKSGNTASLTISGL | | | | | | |
| | | | QAEDEADYYCSSYTSSSTLGLYVFGTGT | | | | | | |
| | | | KVTVL | | | | | | |
| | COVA2- | 1764 | QSVLTQPPSVSGAPGQRVTISCTGSSS | IGHV1-24 | IGHJ5 | IGLV1-40 | IGU3 | 2773 | ATSPAVMSV |
| | 37 | | NIGAGYDVHWYQQLPGTAPKVLIYDN | (Human) | (Human) | (Human) | (Human) | | GWVDP |
| | | | NNRPSGVPDRFSGSKSGTSASLAITGL | | | | | | |
| | | | QAEDEADYYCQSYDSSLSGSVFGGGT | | | | | | |
| | | | KLTVL | | | | | | |
| | COVA2- | 1765 | DIQLTQSPSSLSASVGDRVTITCRASQG | IGHV4-39 | IGHJ3 | IGKV1-17 | IGKJ4 | 2774 | ARQVRQWLE |
| | 38 | | IRNDLGWYQQKPGKAPKRLIYAASSL | (Human) | (Human) | (Human) | (Human) | | DDAFDI |
| | | | QSGVPSRFSGSGSGTEFTLTISSLQPED | | | | | | |
| | | | FATYYCLQHNSYPLTFGGGTKVDIK | | | | | | |
| | COVA2- | 1766 | QSALTQPASVSGSPGQSITISCTGTSSD | IGHV3-53 | IGHJ3 | IGLV2-23 | IGU3 | 2775 | ARAHVDTAM |
| | 39 | | VGSYNLVSWYQQHPGKAPKLMIYEVT | (Human) | (Human) | (Human) | (Human) | | VESGAFDI |
| | | | KRPSGVSNRFSGSKSGNTASLTISGLQ | | | | | | |
| | | | AEDEADYYCCSYAGSSTWVFGGGTKL | | | | | | |
| | | | TVL | | | | | | |
| | COVA2- | 1767 | QSVLTQPASVSGSPGQSITISCTGTSSD | IGHV4-4 | IGHJ5 | IGLV2-23 | IGU3 | 2776 | AGRYCSGGRC |
| | 40 | | VGSYNLVSWYQQHPGKAPKLMIYEAS | (Human) | (Human) | (Human) | (Human) | | GWFDP |
| | | | KRPSGISNRFSGSKSGNTASLTISGLQA | | | | | | |
| | | | EDEADYYCCSYAGSSTWVFGGGTKPT | | | | | | |
| | | | VL | | | | | | |
| | COVA2- | 1768 | DIVMTQSPATLSVSPGERATLSCRASQ | IGHV3-21 | IGHJ6 | IGKV3-15 | IGKJ1 | 2777 | ARVQKDIVVV |
| | 41 | | SVSSNLAWYQQKPGQAPRLLIYGAST | (Human) | (Human) | (Human) | (Human) | | PVALADYYYY |
| | | | RATGIPARFSGSGSGTEFTLTISSLQPE | | | | | | GMDV |
| | | | DFAVYYCQQCYNWPPWTFGQGTRVE | | | | | | |
| | | | FK | | | | | | |
| | COVA2- | 1769 | DIVMTQSPSSLSASVGDRVTITCRASQ | IGHV1-18 | IGHJ4 | IGKV1-17 | IGKJ4 | 2778 | ARFDYGYPYS |
| | 43 | | GIRNDLGWYQQKPGKAPKRLIYAASSL | (Human) | (Human) | (Human) | (Human) | | SWSVLSIDY |
| | | | QSGVPSRFSGSGSGTEFTLTISSLQPED | | | | | | |
| | | | FATYYCLQHNSYPLTFGGGTKLEIK | | | | | | |
| | COVA2- | 1770 | AIRMTQSPSSLSASVGDRVTITCRASQ | IGHV3-30 | IGHJ4 | IGKV1-39 | IGKJ2 | 2779 | AREGSRQWL |
| | 44 | | SISSYLNWYQQKPGKAPKLLIYAASSLQ | (Human) | (Human) | (Human) | (Human) | | VIYFDY |
| | | | SGVPSRFSGSGSGTDFTLTISSLQPEDF | | | | | | |
| | | | ATYYCQQSYTTFIYTFGQGTKLEIK | | | | | | |
| | COVA2- | 1771 | DIVMTQSPGTLSLSPGERATLSCRASQ | IGHV1-2 | IGHJ5 | IGKV3-20 | IGKJ1 | 2780 | ARGDGDYYD |
| | 45 | | SVSSSYLAWYQQKPGQAPRLLIYGASS | (Human) | (Human) | (Human) | (Human) | | SSGYYRPTLY |
| | | | RATGIPDRFSGSGSGTDFTLTISRLEPE | | | | | | NWLDP |
| | | | DFAVYYCQQYGSSPYTFGQGTKVEIK | | | | | | |
| | COVA2- | 1772 | AIRMTQSPSSLSASVGDRVTITCQASQ | IGHV4-39 | IGHJ4 | IGKV1-33 | IGKJ4 | 2781 | ARHPSGLYQL |
| | 46 | | DISNYLNWYQQKPGKAPKLLIYDASNL | (Human) | (Human) | (Human) | (Human) | | LN |
| | | | ETGVPSRFSGSGSGTDFTFTISSLQPED | | | | | | |
| | | | IATYYCQQYDNLLSLTFGGGTKVEIK | | | | | | |
| | COVA2- | 1773 | EIVLTQSPDSLAVSLGERATINCKSSQS | IGHV3-9 | IGHJ4 | IGKV4-1 | IGKJ4 | 2782 | AKVATYYYDR |
| | 47 | | VLYSSNNKNYLAWYQQKPGQPPKLLI | (Human) | (Human) | (Human) | (Human) | | SGYYYGGALD |
| | | | YWASTRESGVPDRFSGSGSGTHFTLTI | | | | | | Y |
| | | | SSLQAEDVAVYYCQQYYSTPPLTFGGG | | | | | | |
| | | | TKVDIK | | | | | | |
| | COVA3- | 1774 | EIVMTQSPSSLSASVGDRVTITCRASQS | IGHV4-59 | IGHJ6 | IGKV1-39 | IGKJ4 | 2783 | ARGPAATYYY |
| | 01 | | MSSYLNWYQQKPGKAPKLLIYAASSL | (Human) | (Human) | (Human) | (Human) | | YMDV |
| | | | QSGVPSRFSGSGSGTDFTLTISSLQPED | | | | | | |
| | | | FATYYCQQSYSTLTFGGGTKVEIK | | | | | | |
| | COVA3- | 1775 | QTVVTQPPSVSVSPGQTARITCSGDAL | IGHV3-23 | IGHJ4 | IGLV3-25 | IGU3 | 2784 | AKEIAVAGCF |
| | 03 | | PKQYAYWYQQKPGQAPVLVIYKDSER | (Human) | (Human) | (Human) | (Human) | | DY |
| | | | PSGIPERFSGSSSGTTVTLTISGVQAED | | | | | | |
| | | | EADYYCQSADSSGTYRVFGGGTKLTVL | | | | | | |
| | COVA3- | 1776 | QPVLTQPPSVSVAPGQTARITCGGNN | IGHV3-33 | IGHJ4 | IGLV3-21 | IGU1 | 2785 | ARVGSVKSTA |
| | 04 | | IGSKSVHWYQQKPGQAPVLVVYDDS | (Human) | (Human) | (Human) | (Human) | | GYDFWSGDP |
| | | | DRPSGIPERFSGSNSGNTATLTISRVEA | | | | | | FDY |
| | | | GDEADYYCQVWDSSSDHYVFGTGTK | | | | | | |
| | | | VTVL | | | | | | |
| | COVA3- | 1777 | NFMLTQPPSASGTPGQRVTISCSGSSS | IGHV1-24 | IGHJ4 | IGLV1-44 | IGU3 | 2786 | ATAYSVDTA |
| | 05 | | NIGSNTVNWYQQLPGTAPKLLIYSNN | (Human) | (Human) | (Human) | (Human) | | MVRGVGY |
| | | | QRPSGVPDRFSGSKSGTSASLAISGLQ | | | | | | |
| | | | SEDEADYYCAAWDDSLNGPHWVFGG | | | | | | |
| | | | GTKLTVL | | | | | | |
| | COVA3- | 1778 | DVVMTQSPDSLAVSLGERATINCKSS | IGHV1-69 | IGHJ4 | IGKV4-1 | IGKJ4 | 2787 | ARDAPDYYDS |
| | 06 | | QSVLYSSNNKNYLAWYQQKPGQPPKL | (Human) | (Human) | (Human) | (Human) | | SGPTYFDY |
| | | | LIYWASTRESGVPDRFSGSGSGTDFTL | | | | | | |
| | | | TISSLQAEDVAVYYCQQYYSTPLTFGG | | | | | | |
| | | | GTKVDIK | | | | | | |
| | COVA3- | 1779 | EIVLTQSPGTLSLSPGERATLSCRASQS | IGHV3-9 | IGHJ6 | IGKV3-20 | IGKJ3 | 2788 | AKMGPDPAH |
| | 07 | | VSSSYLAWYQQKPGQAPRLLIYGASSR | (Human) | (Human) | (Human) | (Human) | | DYGRKNDAF |
| | | | ATGIPDRFSGSGSGTDFTLTISRLEPED | | | | | | DI |
| | | | FAVYYCQQYGSSPFTFGPGTKLEIK | | | | | | |
| | COVA3- | 1780 | DIVMTQSPSSLSASVGDRVTITCRASQ | IGHV4-59 | IGHJ6 | IGKV1-39 | IGKJ4 | 2789 | AKAEPEVGGY |
| | 08 | | SISSYLNWYQQKPGKAPKLLIYAASSLQ | (Human) | (Human) | (Human) | (Human) | | DYYMDV |
| | | | SGVPSRFSGSGSGTDFTLTISSLQPEDF | | | | | | |
| | | | ATYYCQQSYSTPLTFGPGTKLEIK | | | | | | |
| | COVA3- | 1781 | DIVMTQSPSSLSASVGDRVTITCRASQ | IGHV3-9 | IGHJ3 | IGKV1-39 | IGKJ4 | 2790 | ARGPAATYYY |
| | 09 | | SMSSYLNWYQQKPGKAPKLLIYAASSL | (Human) | (Human) | (Human) | (Human) | | YMDV |
| | | | QSGVPSRFSGSGSGTDFTLTISSLQPED | | | | | | |
| | | | FATYYCQQSYSTLTFGGGTKLEIK | | | | | | |
| | COVA3- | 1782 | DIVMTQSPDSLAVSLGERATINCKSSQ | IGHV5-51 | IGHJ6 | IGKV4-1 | IGKJ5 | 2791 | ARRGYTYGAD |
| | 10 | | SVLYSSNNKNYLAWYQQKPGQPPKLLI | (Human) | (Human) | (Human) | (Human) | | FYGLDV |
| | | | YWASTRESGVPDRFSGSGSGTDFTLTI | | | | | | |
| | | | SSLQAEDVAVYYCQQYYSTPITFGQGT | | | | | | |
| | | | RLEIK | | | | | | |
| | CR3022 | 1783 | DIQLTQSPDSLAVSLGERATINCKSSQS | IGHV5-51 | IGHJ6 | IGKV4-1 | IGKJ1 | 2792 | AGGSGISTPM |
| | | | VLYSSINKNYLAWYQQKPGQPPKLLIY | (Human) | (Human) | (Human) | (Human) | | DV |
| | | | WASTRESGVPDRFSGSGSGTDFTLTIS | | | | | | |
| | | | SLQAEDVAVYYCQQYYSTPYTFGQGT | | | | | | |
| | | | KVEIK | | | | | | |
| | CV-X1- | | ND | IGHV3-53 | IGHJ6 | ND | ND | 2793 | ARDLSEGGM |
| | 126 | | | (Human) | (Human) | | | | DV |
| | CV-X2- | | ND | IGHV1-69 | IGHJ6 | ND | ND | 2794 | ATRKETTVTTS |
| | 106 | | | (Human) | (Human) | | | | LVYGMDV |
| | CV05-163 | | ND | IGHV1-2 | IGHJ6 | ND | ND | 2795 | AREVMVRGA |
| | | | | (Human) | (Human) | | | | LPPYGMDV |
| | CV07-200 | | ND | IGHV1-2 | IGHJ6 | ND | ND | 2796 | ARGPFYYDNS |
| | | | | (Human) | (Human) | | | | GTLGGLDV |
| | CV07-209 | | ND | IGHV3-11 | IGHJ4 | ND | ND | 2797 | ARDGVIPPRF |
| | | | | (Human) | (Human) | | | | DY |
| | CV07-222 | | ND | IGHV1-2 | IGHJ3 | ND | ND | 2798 | ARGPYYYDSS |
| | | | | (Human) | (Human) | | | | GSLGAFDI |
| | CV07-250 | | ND | IGHV1-18 | IGHJ6 | ND | ND | 2799 | AGSDNYGFPY |
| | | | | (Human) | (Human) | | | | NGMDV |
| | CV07-255 | | ND | IGHV1-2 | IGHJ4 | ND | ND | 2800 | ARDSRFSYVN |
| | | | | (Human) | (Human) | | | | GEFDY |
| | CV07-262 | | ND | IGHV1-2 | IGHJ6 | ND | ND | 2801 | ARVGWYDFG |
| | | | | (Human) | (Human) | | | | TPGDYYYYYG |
| | | | | | | | | | MDV |
| | CV07-270 | | ND | IGHV1-2 | IGHJ6 | ND | ND | 2802 | ARVFGPGLDC |
| | | | | (Human) | (Human) | | | | SSTSCYTYGM |
| | | | | | | | | | DV |
| | CV07-283 | | ND | IGHV1-2 | IGHJ6 | ND | ND | 2803 | VRGPFYYDSS |
| | | | | (Human) | (Human) | | | | GPLGGMDV |
| | CV07-287 | | ND | IGHV1-58 | IGHJ3 | ND | ND | 2804 | AAPYCSSTNC |
| | | | | (Human) | (Human) | | | | YDAFDI |
| | CV07-315 | | ND | IGHV3-9 | IGHJ6 | ND | ND | 2805 | AKDFLWDLH |
| | | | | (Human) | (Human) | | | | PPRYYGMDV |
| | CV1 | 1784 | QSVLTQPPSASGTPGQRVTISCSGSSS | IGHV4- | IGHJ2 | IGLV1-44 | IGU3 | 2806 | ARTPLSLRLRY |
| | | | NIGSNTVNWYQQLPGTAPKLLIYSNN | 382 | (Human) | (Human) | (Human) | | NWYFDL |
| | | | QRPSGVPDRFSGSKSGTSASLAISGLQ | (Human) | | | | | |
| | | | SEDEADYYCAAWDDSLNGPVFGGGT | | | | | | |
| | | | KLTVL | | | | | | |
| | CV10 | 1785 | EIVLTQSPGTLSLSPGERATLSCRASQS | IGHV4-59 | IGHJ4 | IGKV3-20 | IGKJ1 | 2807 | ARGFDY |
| | | | VSSIYLAWYQQKPGQAPRLLIYGASSR | (Human) | (Human) | (Human) | (Human) | | |
| | | | ATGIPDRFSGSGSGTDFTLTISRLEPED | | | | | | |
| | | | FAVYYCQQYAGSPWTFGQGTKVEIK | | | | | | |
| | CV11 | 1786 | EIVLTQSPATLSLSPGERATLSCRASQS | IGHV4-31 | IGHJ4 | IGKV3-11 | IGKJ3 | 2808 | ARETTGHFDY |
| | | | VSSYLAWYQQKPGQAPRLLIYDASNR | (Human) | (Human) | (Human) | (Human) | | |
| | | | ATGIPARFSGSGSGTDFTLTISSLEPED | | | | | | |
| | | | FAVYYCQQRSNWPPIFTFGPGTKVDIK | | | | | | |
| | CV12 | 1787 | DVVMTQSPLSLPVTLGQPATISCRSSQ | IGHV3-30 | IGHJ4 | IGKV2-30 | IGKJ1 | 2809 | VRGGVSGPN |
| | | | SLVYSDGNTYVNWFQQRPGQSPRRLI | (Human) | (Human) | (Human) | (Human) | | SFDM |
| | | | YQVSIRASGVPDRFSGSGSGTDFALKIS | | | | | | |
| | | | RVEAEDVGVYYCMQGTHWPVTFGQ | | | | | | |
| | | | GTKVEIK | | | | | | |
| | CV13 | 1788 | DIQMTQSPSSLSASVGDRVTITCRASQ | IGHV7-4- | IGHJ4 | IGKV1-39 | IGKJ3 | 2810 | ARASARPGVA |
| | | | NIDNYLNWYQQKPGKAPKLLIYAASRL | 1 | (Human) | (Human) | (Human) | | TNLDF |
| | | | HSGVPSRFSGSGSGTDFTLIISSLQPED | (Human) | | | | | |
| | | | LATYYCQQSYSNPLTFGPGTKVDIR | | | | | | |
| | CV15 | 1789 | QSALTQPRSVSGSPGQSVTISCTGTSS | IGHV3-7 | IGHJ6 | IGLV2-11 | IGU3 | 2811 | ARDFNSYQLL |
| | | | DVGGYNYVSWYQQHPGKAPKLMIYD | (Human) | (Human) | (Human) | (Human) | | WYYYYGMDV |
| | | | VSKRPSGVPDRFSGSKSGNTASLTISGL | | | | | | |
| | | | QAEDEADYYCCSYAGSYTWVFGGGTK | | | | | | |
| | | | LTVL | | | | | | |
| | CV16 | 1790 | EIVLTQSPGTLSLSPGERATLSCRASQS | IGHV5-51 | IGHJ6 | IGKV3-20 | IGKJ1 | 2812 | ARQSSFYSSG |
| | | | VSSSYLAWYQQKPGQAPRLLIYGASSR | (Human) | (Human) | (Human) | (Human) | | WYSYGMDV |
| | | | ATGIPDRFSGSGSGTDFTLTISRLEPED | | | | | | |
| | | | FAVYYCQQYGSSRGTFGQGTKVEIK | | | | | | |
| | CV17 | 1791 | QSALTQPASVSGSPGQSITISCTGTSSD | IGHV1-2 | IGHJ5 | IGLV2-23 | IGU1 | 2813 | ARVDYGSGSY |
| | | | VGSYNLVSWYQQHPGKAPKLMIYEGS | (Human) | (Human) | (Human) | (Human) | | GWGWFDP |
| | | | KRPSGVSNRFSGSKSGNTASLTISGLQ | | | | | | |
| | | | AEDEADYYCCSYAGSSTYVFGTGTKVT | | | | | | |
| | | | VL | | | | | | |
| | CV18 | 1792 | QSVLTQPPSVSAAPGQKVTISCSGSSS | IGHV1-24 | IGHJ5 | IGLV1-51 | IGU3 | 2814 | ATTSPIVGAIT |
| | | | NIGNNYVSWYQQLPGTAPKLLIYDNN | (Human) | (Human) | (Human) | (Human) | | WFDP |
| | | | KRPSGIPDRFSGSKSGTSATLGITGLQT | | | | | | |
| | | | GDEADYYCGTWDSSLSAGPVFGGGT | | | | | | |
| | | | KLTVL | | | | | | |
| | CV19 | 1793 | EIVLTQSPGTLSLSPGERATLSCRASQS | IGHV1-2 | IGHJ6 | IGKV3-20 | IGKJ2 | 2815 | AREYYYDSSV |
| | | | VSSSYLAWYQQKPGQAPRLLIYGASSR | (Human) | (Human) | (Human) | (Human) | | YPYYYYAMDV |
| | | | ATGIPDRFSGSGSGTDFTLTISRLEPED | | | | | | |
| | | | FAVYYCQQYGSSPPKYTFGQGTKLEIK | | | | | | |
| | CV2 | 1794 | EIVMTQSPATLSVSPGERATLSCRASQ | IGHV3-30 | IGHJ4 | IGKV3-15 | IGKJ4 | 2816 | ARVRGSYYLF |
| | | | SVSSNLAWYQQKPGQAPRLLIYGAST | (Human) | (Human) | (Human) | (Human) | | DY |
| | | | RATGIPARFSGSGSGTEFTLTISSLQSE | | | | | | |
| | | | DFAVYYCQQYNNWPPSLTFGGGTKV | | | | | | |
| | | | EIK | | | | | | |
| | CV21 | 1795 | EIVLTQSPATLSLSPGERATLSCRASQS | IGHV3-15 | IGHJ4 | IGKV3-11 | IGKJ4 | 2817 | TTDRVYDYIW |
| | | | VSSYLAWYQQKPGQAPRLLIYDASNR | (Human) | (Human) | (Human) | (Human) | | GSYRYLDY |
| | | | ATGIPARFSGSGSGTDFTLTISSLEPED | | | | | | |
| | | | FAVYYCQQRSNWPLTFGGGTKVEIK | | | | | | |
| | CV22 | 1796 | QLVLTQSPSASASLGASVKLTCTLSSGH | IGHV3-21 | IGHJ4 | IGLV4-69 | IGU3 | 2818 | ARDRESYDILT |
| | | | SSYAIAWHQQQPEKGPRYLMKLNSD | (Human) | (Human) | (Human) | (Human) | | GYSMEGCFD |
| | | | GSHSKGDGIPDRFSGSSSGAERYLTISS | | | | | | Y |
| | | | LQSEDEADYYCQTWGTGIRVFGGGTK | | | | | | |
| | | | LTVL | | | | | | |
| | CV23 | 1797 | SYELTQPPSVSVSPGQTARITCSGDALP | IGHV1-3 | IGHJ3 | IGLV3-25 | IGU3 | 2819 | ARVWGYCSG |
| | | | KQYAYWYQQKPGQAPVLVIYKDSERP | (Human) | (Human) | (Human) | (Human) | | GSCYVDAFDI |
| | | | SGIPERFSGSSSGTTVTLTISGVQAEDE | | | | | | |
| | | | ADYYCQSADSSGTYVVFGGGTKLTVL | | | | | | |
| | CV24 | 1798 | QSVLTQPPSVSAAPGQKVTISCSGSSS | IGHV1-24 | IGHJ5 | IGLV1-51 | IGU1 | 2820 | ATAPPYSPPSS |
| | | | NIGNNYVSWYQQLPGTAPKLLIYDNN | (Human) | (Human) | (Human) | (Human) | | WFDP |
| | | | KRPSGIPDRFSGSKSGTSATLGITGLQT | | | | | | |
| | | | GDEADYYCGTWDSSLSASYVFGTGTK | | | | | | |
| | | | VTVL | | | | | | |
| | CV25 | 1799 | EIVMTQSPATLSVSPGERATLSCRASQ | IGHV4-30 | IGHJ6 | IGKV3-15 | IGKJ2 | 2821 | ARDHHYDFW |
| | | | SVSSNLAWYQQKPGQAPRLLIYGAST | (Human) | (Human) | (Human) | (Human) | | SGYSSYYYYG |
| | | | RATGIPARFSGSGSGTEFTLTISSLQSE | | | | | | MDV |
| | | | DFAVYYCQQYNNWPYTFGQGTKLEIK | | | | | | |
| | CV26 | 1800 | DIQMTQSPSSLSASVGDRVTITCRASQ | IGHV3-30 | IGHJ6 | IGKV1-17 | IGKJ3 | 2822 | ARDEAYYDILT |
| | | | GIRNDLGWYQQKPGKAPKRLIYAASSL | 3 | (Human) | (Human) | (Human) | | GYINAPKNYY |
| | | | QSGVPSRFSGSGSGTEFTLTISSLQPED | (Human) | | | | | YYGMDV |
| | | | FATYYCLQHNSYPFTFGPGTKVDIK | | | | | | |
| | CV27 | 1801 | QSALTQPASVSGSPGQSITISCTGTSSD | IGHV3-30 | IGHJ6 | IGLV2-14 | IGU1 | 2823 | ARSFGGSYYY |
| | | | VGGYNYVSWYQQHPGKAPKLMIYDV | (Human) | (Human) | (Human) | (Human) | | GMDV |
| | | | SNRPSGVSNRFSGSKSGNTASLTISGL | | | | | | |
| | | | QAEDEADYYCSSYTSSSTPYVFGTGTK | | | | | | |
| | | | VTVL | | | | | | |
| | CV3 | 1802 | DIQMTQSPSSLSASVGDRVTITCRASQ | IGHV7-4- | IGHJ4 | IGKV1-39 | IGKJ3 | 2824 | ARASARPGVA |
| | | | NIDNYLNWYQQKPGKAPKLLIYAASRL | 1 | (Human) | (Human) | (Human) | | TNLDF |
| | | | HSGVPSRFSGSGSGTDFTLIISSLQPED | (Human) | | | | | |
| | | | LATYYCQQSYSNPLTFGPGTKVDIR | | | | | | |
| | CV30 | 1803 | EIVLTQSPGTLSLSPGERATLSCRASQS | IGHV3-53 | IGHJ6 | IGKV3-20 | IGKJ2 | 2825 | ARDLDVSGG |
| | | | VSSSYLAWYQQKPGQAPRLLIYGASSR | (Human) | (Human) | (Human) | (Human) | | MDV |
| | | | ATGIPDRFSGSGSGTDFTLTISRLEPED | | | | | | |
| | | | FAVYYCQQYGSSPQTFGQGTKLEIK | | | | | | |
| | CV31 | 1804 | QSVLTQPPSVSAAPGQKVTISCSGSSS | IGHV1-24 | IGHJ5 | IGLV1-51 | IGU1 | 2826 | ATAPPYSPPSS |
| | | | NIGNNYVSWYQQLPGTAPKLLIYDNN | (Human) | (Human) | (Human) | (Human) | | WFDP |
| | | | KRPSGIPDRFSGSKSGTSATLGITGLQT | | | | | | |
| | | | GDEADYYCGTWDSSLSASYVFGTGTK | | | | | | |
| | | | VTVL | | | | | | |
| | CV32 | 1805 | QSVLTQPPSVSAAPGQKVTISCSGSSS | IGHV1-2 | IGHJ4 | IGLV1-51 | IGU3 | 2827 | AREARDYYGS |
| | | | NIGNNYVSWYQQLPGTAPKLLIYDNN | (Human) | (Human) | (Human) | (Human) | | GSLDY |
| | | | KRPSGIPDRFSGSKSGTSATLGITGLQT | | | | | | |
| | | | GDEADYYCGTWDSSLSAVVFGGGTKL | | | | | | |
| | | | TVL | | | | | | |
| | CV33 | 1806 | QSVLTQPPSVSGAPGQRVTISCTGSSS | IGHV1-18 | IGHJ6 | IGLV1-40 | IGU3 | 2828 | ARDSVAGIYY |
| | | | NIGAGYDVHWYQQLPGTAPKLLIYGN | (Human) | (Human) | (Human) | (Human) | | YYGMDV |
| | | | SNRPSGVPDRFSGSKSGTSASLAITGL | | | | | | |
| | | | QAEDEADYYCQSYDSSLSGPVVFGGG | | | | | | |
| | | | TKLTVL | | | | | | |
| | CV34 | 1807 | SYELTQPHSVSVATAQMARITCGGNN | IGHV3-30 | IGHJ6 | IGLV3-12 | IGU3 | 2829 | ARSYGGSYYY |
| | | | IGSKAVHWYQQKPGQDPVLVIYSDSN | 3 | (Human) | (Human) | (Human) | | GMDV |
| | | | RPSGIPERFSGSNPGNTATLTISRIEAG | (Human) | | | | | |
| | | | DEADYYCQVWDSSSDHVVFGGGTKL | | | | | | |
| | | | TVL | | | | | | |
| | CV35 | 1808 | QSVLTQPPSASGTPGQRVTISCSGSSS | IGHV4-38 | IGHJ2 | IGLV1-44 | IGU3 | 2830 | ARTPLSLRLRY |
| | | | NIGSNTVNWYQQLPGTAPKLLIYSNN | (Human) | (Human) | (Human) | (Human) | | NWYFDL |
| | | | QRPSGVPDRFSGSKSGTSASLAISGLQ | | | | | | |
| | | | SEDEADYYCAAWDDSLNGPVFGGGT | | | | | | |
| | | | KLTVL | | | | | | |
| | CV36 | 1809 | SYELTQPPSVSVSPGQTARITCSGDALP | IGHV1-2 | IGHJ6 | IGLV3-25 | IGU1 | 2831 | ARDLTTTAGT |
| | | | KQYAYWYQQKPGQAPVLVIYKDTERP | (Human) | (Human) | (Human) | (Human) | | DYYYGMDV |
| | | | SGIPERFSGSSSGTTVTLTISGVQAEDE | | | | | | |
| | | | ADYYCQSADSSGTYMIFGTGTKVTVL | | | | | | |
| | CV37 | 1810 | DIQMTQSPSSLSASVGDRVTITCQASQ | IGHV1-18 | IGHJ4 | IGKV1-33 | IGKJ3 | 2832 | ARARVAYDYl |
| | | | DISNYLNWYQQKPGKAPKLLIYDASNL | (Human) | (Human) | (Human) | (Human) | | WGSYRYKAF |
| | | | ETGVPSRFSGSGSGTDFTFTISSLQPED | | | | | | DY |
| | | | IATYYCQQYDNLPRFGPGTKVDIKR | | | | | | |
| | CV38 | 1811 | EIVLTQSPATLSLSPGERATLSCRASQS | IGHV3-30 | IGHJ4 | IGKV3-11 | IGKJ5 | 2833 | ARAQTAHYSS |
| | | | VSSYLAWYQQKPGQAPRLLIYDASNR | (Human) | (Human) | (Human) | (Human) | | SFDY |
| | | | ATGIPARFSGSGSGTDFTLTISSLEPED | | | | | | |
| | | | FAVYYCQQRSNWPPITFGQGTRLEIK | | | | | | |
| | CV38-113 | | ND | IGHV3-53 | IGHJ4 | ND | ND | 2834 | ARGGRLADA |
| | | | | (Human) | (Human) | | | | AGDY |
| | CV38-139 | | ND | IGHV3-53 | IGHJ4 | ND | ND | 2835 | ARGHYDLFDY |
| | | | | (Human) | (Human) | | | | |
| | CV38-142 | | ND | IGHV5-51 | IGHJ4 | ND | ND | 2836 | ARIRGVYSSG |
| | | | | (Human) | (Human) | | | | WIGGDY |
| | CV38-183 | | ND | IGHV3-53 | IGHJ6 | ND | ND | 2837 | ARGDGWDN |
| | | | | (Human) | (Human) | | | | YYYGMDV |
| | CV38-221 | | ND | IGHV3-66 | IGHJ4 | ND | ND | 2838 | ARGFGDYYFD |
| | | | | (Human) | (Human) | | | | Y |
| | CV39 | 1812 | DVVMTQSPLSLPVTLGQPASISCRSSQ | IGHV3-30 | IGHJ3 | IGKV2-30 | IGKJ1 | 2839 | VRGGVSGPN |
| | | | SLVYSDGNTYVNWFQQRPGQSPRRLI | (Human) | (Human) | (Human) | (Human) | | AFDI |
| | | | YKVSNRDSGVPDRFSGSGSGTDFALKI | | | | | | |
| | | | SRVEAEDVGVYYCMQGTHWPVTFGQ | | | | | | |
| | | | GTKVEIK | | | | | | |
| | CV4 | 1813 | DIQMTQSPSTLSASVGDRVTITCRASQ | IGHV3-30 | IGHJ4 | IGKV1-5 | IGKJ2 | 2840 | ARSISGSYLGA |
| | | | SISSWLAWYQQKPGKAPKLLIYKASSL | (Human) | (Human) | (Human) | (Human) | | FDY |
| | | | ESGVPSRFSGSGSGTEFTLTISSLQPDD | | | | | | |
| | | | FATYYCQQYNSYTFGQGTKLEIK | | | | | | |
| | CV40 | 1814 | DIQMTQSPSSLSASVGDRVTITCRASQ | IGHV1-18 | IGHJ3 | IGKV1-17 | IGKJ4 | 2841 | ARVGLWWL |
| | | | GIRNDLGWYQQKPGKAPKRLIYAASSL | (Human) | (Human) | (Human) | (Human) | | GHPDAFDI |
| | | | QSGVPSRFSGSGSGTEFTLTISSLQPED | | | | | | |
| | | | FATYYCLQHNSYPLTFGGGTKVEIK | | | | | | |
| | CV41 | 1815 | EIVMTQSPATLSVSPGERATLSCRASQ | IGHV3-30 | IGHJ4 | IGKV3-15 | IGKJ4 | 2842 | ARTKGGSYFA |
| | | | SVSSNLAWYQQKPGQAPRLLIYGAST | (Human) | (Human) | (Human) | (Human) | | PFDY |
| | | | RATGIPARFSGSGSGTEFTLTISSLQSE | | | | | | |
| | | | DFAVYYCQQYNNWPLTFGGGTKVEIK | | | | | | |
| | CV42 | 1816 | DIQMTQSPSSLSASVGDRVTITCRASQ | IGHV1-18 | IGHJ4 | IGKV1-39 | IGKJ3 | 2843 | ARDRGYAATF |
| | | | SISSYLNWYQQKPGKAPKFLIYAASSLQ | (Human) | (Human) | (Human) | (Human) | | GVFDY |
| | | | SGVPSRFSGSGSGTDFTLTISSLQPEDF | | | | | | |
| | | | ATYYCQQTYITAFTFGPGTKVDIK | | | | | | |
| | CV43 | 1817 | NFMLTQPHSVSESPGKTVTISCTGSSG | IGHV3-30 | IGHJ4 | IGLV6-57 | IGU3 | 2844 | ARVTVVHFDY |
| | | | SIASNYVQWYQQRPGSAPTTVIYEDN | (Human) | (Human) | (Human) | (Human) | | |
| | | | QRPSGVPDRFSGSIDSSSNSASLTISGL | | | | | | |
| | | | KTEDEADYYCQSYDSSNWVFGGGTKL | | | | | | |
| | | | TVL | | | | | | |
| | CV44 | 1818 | SYELTQPPSVSVSPGQTARITCSGDALP | IGHV1-46 | IGHJ6 | IGLV3-25 | IGU3 | 2845 | ARDLTSTSSSP |
| | | | KQYAYWYQQKPGQAPVVVIYKDSERP | (Human) | (Human) | (Human) | (Human) | | YSYYYGMDV |
| | | | SGIPERFSGSSSGTTVTLTISGVQAEDE | | | | | | |
| | | | ADYYCQSADSSGTYVVFGGGTKLTVL | | | | | | |
| | CV45 | 1819 | QSVLTQPPSVSGAPGQRVTISCTGSSS | IGHV1-18 | IGHJ5 | IGLV1-40 | IGU1 | 2846 | ARVTVEAIFG |
| | | | NIGAGYDVHWYQQLPGTAPKLLIYGN | (Human) | (Human) | (Human) | (Human) | | VVILPLKNWF |
| | | | SNRPSGVPDRFSGSKSGTSASLAITGL | | | | | | DP |
| | | | QAEDEADYYCQSYDSSLTLYVFGTGTK | | | | | | |
| | | | VTVL | | | | | | |
| | CV46 | 1820 | DVVMTQSPLSLPVTLGQPASISCRSSQ | IGHV3-30 | IGHJ4 | IGKV2-30 | IGKJ1 | 2847 | VRGGVSGPN |
| | | | SLVYSDGNTYVNWFQQRPGQSPRRLI | (Human) | (Human) | (Human) | (Human) | | SFDM |
| | | | YQVSIRASGVPDRFSGSGSGTDFALKIS | | | | | | |
| | | | RVEAEDVGVYYCMQGTHWPVTFGQ | | | | | | |
| | | | GTKVEIK | | | | | | |
| | CV47 | 1821 | DIQMTQSPSSLSASVGDRVTITCRASQ | IGHV1-18 | IGHJ3 | IGKV1-17 | IGKJ4 | 2848 | ARVGLWWL |
| | | | GIRNDLGWYQQKPGKAPKRLIYAASSL | (Human) | (Human) | (Human) | (Human) | | GHPDVFDI |
| | | | QSGVPSRFSGSGSGTEFTLTISSLQPED | | | | | | |
| | | | FATYYCLQHNSYPLTFGGGTKVEIK | | | | | | |
| | CV48 | 1822 | DVVMTQSPLSLPVTLGQPASISCRSSQ | IGHV1-69 | IGHJ4 | IGKV2-30 | IGKJ1 | 2849 | ARDLVEDTA |
| | | | SLVYSDGNTYLNWFQQRPGQSPRRLI | (Human) | (Human) | (Human) | (Human) | | MVTGAAAGT |
| | | | YKVSNRDSGVPDKFSGSGSGTDFTLKI | | | | | | |
| | | | SRVEAEDVGVYYCMQGTHWPPTFGQ | | | | | | |
| | | | GTKVEIK | | | | | | |
| | CV5 | 1823 | DIVMTQSPDSLAVSLGERATINCKSSQ | IGHV1-46 | IGHJ3 | IGKV4-1 | IGKJ2 | 2850 | ARAGRRYSSS |
| | | | NVLYSSNNKNYLAWYQQKPGQPPKLL | (Human) | (Human) | (Human) | (Human) | | DDGAFDI |
| | | | IYWASTRESGVPDRFSGSGSGTDFTLTI | | | | | | |
| | | | SSLQAEDVAVYYCQQYYITPYTFGQGT | | | | | | |
| | | | KLEIK | | | | | | |
| | CV50 | 1824 | SYELTQPPSVSVSPGQTARITCSGDALP | IGHV3-33 | IGHJ4 | IGLV3-10 | IGU1 | 2851 | ARDIMFGDD |
| | | | KKYAYWYQQKSGQAPVLVIYEDSKRP | (Human) | (Human) | (Human) | (Human) | | WLQKQPDY |
| | | | SGIPERFSGSSSGTMATLTISGAQVED | | | | | | |
| | | | EADYYCYSTDSSGNLYVFGTGTKVTVL | | | | | | |
| | CV7 | 1825 | DIQMTQSPSTLSASVGDRVTITCRASQ | IGHV3-30 | IGHJ4 | IGKV1-5 | IGKJ2 | 2852 | ARSISGSYLGA |
| | | | SISSWLAWYQQKPGKAPKLLIYKASSL | (Human) | (Human) | (Human) | (Human) | | FDY |
| | | | ESGVPSRFSGSGSGTEFTLTISSLQPDD | | | | | | |
| | | | FATYYCQQYNSYTFGQGTKLEIK | | | | | | |
| | CV8 | 1826 | EIVLTQSPGTLSLSPGERATLSCRASQS | IGHV1-18 | IGHJ6 | IGKV3-20 | IGKJ5 | 2853 | ARLVPTWASY |
| | | | VSSSYLAWYQQKPGQAPRLLIYGASSR | (Human) | (Human) | (Human) | (Human) | | YDFWSGYPG |
| | | | ATGIPDRFSGSGSGTDFTLTISRLEPED | | | | | | GYGMDV |
| | | | FAVYYCQQYGSSPGTFGQGTRLEIK | | | | | | |
| | CV9 | 1827 | QSALTQPASVSGSPGQSITISCTGTSSD | IGHV4-39 | IGHJ2 | IGLV2-14 | IGU3 | 2854 | ATHIVVVTAT |
| | | | VGGYNYVSWYQQHPGKAPKLMIYDV | (Human) | (Human) | (Human) | (Human) | | PNWYFDL |
| | | | SNRPSGVSNRFSGSKSGNTASLTISGL | | | | | | |
| | | | QAEDEADYYCSSYTSISTWVFGGGTKL | | | | | | |
| | | | TVL | | | | | | |
| | EY6A | 1828 | DIQMTQSPSSLSASVGDRVTITCRASQ | IGHV3-30 | IGHJ4 | IGKV1-39 | IGKJ4 | 2855 | AKDGGKLWV |
| | | | SISSYLNWYQQKPGKAPKLLIYAASSLQ | 3 | (Human) | (Human) | (Human) | | YYFDY |
| | | | SGVPSRFSGSGSGTDFTLTISSLQPEDF | (Human) | | | | | |
| | | | ATYYCQQSYSTLALTFGGGTKVEIK | | | | | | |
| | Fab 2-4 | 1829 | QSALTQPPSASGSPGQSVTISCTGTSS | IGHV1-2 | IGHJ6 | IGLV2-8 | IGU3 | 2856 | ARDRSWAVV |
| | | | DVGGYNYVSWYQQHPGKAPKLMIYE | (Human) | (Human) | (Human) | (Human) | | YYYMDV |
| | | | VSKRPSGVPDRFSGSKSGNTASLTVSG | | | | | | |
| | | | LQAEDEADYYCSSYAGSNNLVFGGGT | | | | | | |
| | | | KLTVL | | | | | | |
| | FnC1t1p2_ | 1830 | EIVLTQSPGTLSLSPGERATLSCRASQS | IGHV1-8 | IGHJ4 | IGKV3-20 | IGKJ1 | 2857 | ARATTDCSST |
| | A5 | | VSSSYLAWYQQKPGQAPRLLIYGASSR | (Human) | (Human) | (Human) | (Human) | | SCWSLDFWS |
| | | | ATGIPDRFSGSGSGTDFTLTISRLEPED | | | | | | GYYTGGREKI |
| | | | FAVYYCQQYGSSPGTFGQGTKVEIK | | | | | | FD |
| | FnC1t2p1_ | 1831 | DIQMTQSPSSLSASVGDRVTITCQASQ | IGHV7-4- | IGHJ5 | IGKV1-33 | IGKJ4 | 2858 | ARSLRGANLV |
| | D4 | | DVSNYLNWYQQQPGKAPKLLIYDAFN | 1 | (Human) | (Human) | (Human) | | P |
| | | | LETGVPSRFSGSGSGTDFTFTISSLQPE | (Human) | | | | | |
| | | | DIATYYCQQYDNLPLTFGGGTKVEIK | | | | | | |
| | FnC1t2p1_ | 1832 | DIQMTQSPSSLSASVGDRVTITCQASQ | IGHV7-4- | IGHJ5 | IGKV1-33 | IGKJ4 | 2859 | ARSLRGANLV |
| | G5 | | DVSNYLNWYQQQPGKAPKLLIYDAFN | 1 | (Human) | (Human) | (Human) | | P |
| | | | LETGVPSRFSGSGSGTDFTFTISSLQPE | (Human) | | | | | |
| | | | DIATYYCQQYDNLPLTFGGGTKVEIK | | | | | | |
| | H014 | | ND | ND | ND | ND | ND | | ND |
| | H11-D4 | | N/A | IGHV3-3 | IGHJ4 | N/A | N/A | 2860 | ARTENVRSLL |
| | | | | (Alpaca) | (Alpaca) | | | | SDYATWPYDY |
| | H11-H4 | | N/A | IGHV3-3 | IGHJ4 | N/A | N/A | 2861 | AQTHYVSYLL |
| | | | | (Alpaca) | (Alpaca) | | | | SDYATWPYDY |
| | H4 | 1833 | DIQMTQSPLSLPVTPGEPASISCRSSQS | IGHV1-2 | IGHJ2 | IGKV2-40 | IGKJ4 | 2862 | ARVPYCSSTSC |
| | | | LLDSDDGNTYLDWYLQKPGQSPQLLIY | (Human) | (Human) | (Human) | (Human) | | HRDWYFDL |
| | | | TLSYRASGVPDRFSGSGSGTDFTLKISR | | | | | | |
| | | | VEAEDVGVYYCMQRIEFPLTFGGGTK | | | | | | |
| | | | VEIK | | | | | | |
| | HbnC2t1p2_ | 1834 | EIVLTQSPATLSLSPGERATLSCRASLS | IGHV3-33 | IGHJ6 | IGKV3-11 | IGKJ1 | 2863 | ARAARRPVVT |
| | D9 | | LSSYLAWYQQKPGQAPRLLIYDASNRA | (Human) | (Human) | (Human) | (Human) | | DTMAYYMDV |
| | | | TGIPARFSGSGSGTDFTLTISSLEPEDF | | | | | | |
| | | | AVYYCQQRSNWPPTWTFGQGTKAEIK | | | | | | |
| | HbnC3t1p1_ | 1835 | EIVLTQSPGTLSLSPGERATLSCRASQS | IGHV1-58 | IGHJ3 | IGKV3-20 | IGKJ1 | 2864 | AAPHCSSTICY |
| | C6 | | VSSSYLAWYQQKPGQAPRLLIYGASSR | (Human) | (Human) | (Human) | (Human) | | DGFDI |
| | | | ATGIPDRFSGSGSGTDFTLTISRVEPED | | | | | | |
| | | | FAVYYCQQYGSSPWTFGQGTKVEIK | | | | | | |
| | HbnC3t1p1_ | 1836 | DIQMTQSPSTVSASVGDRVTITCRASQ | IGHV3-30 | IGHJ5 | IGKV1-5 | IGKJ4 | 2865 | AKGGDYEWE |
| | F4 | | SIDNWLAWYQEKPGKAPKVLIYKASSL | (Human) | (Human) | (Human) | (Human) | | LLES |
| | | | ESGVPSRFSGRGSGTEFTLTISSLQPGD | | | | | | |
| | | | FATYYCQHYHSFPLTFGGGTKVDIK | | | | | | |
| | HbnC3t1p1_ | 1837 | EIVLTQSPGTLSLSPGERATLSCRASQS | IGHV3-66 | IGHJ4 | IGKV3-20 | IGKJ1 | 2866 | ARDFGDFFFD |
| | G4 | | VSSYLAWYQQKPGQAPRLLIYGVSSRA | (Human) | (Human) | (Human) | (Human) | | Y |
| | | | TGIPDRFSGSGSGTDFTLTISRLEPEDF | | | | | | |
| | | | AVYYCQQYGSSPRTFGQGTKVEIK | | | | | | |
| | HbnC3t1p2_ | 1838 | EIVLTQSPGTLSLSPGERATLSCRASQS | IGHV3-66 | IGHJ4 | IGKV3-20 | IGKJ2 | 2867 | ARDYGDYFFD |
| | B10 | | VSSYLAWYQQKPGQAPRLLISGASSRA | (Human) | (Human) | (Human) | (Human) | | Y |
| | | | AGIPDRFSGSGSGTDFTLTINRLEPEDF | | | | | | |
| | | | AVYYCQQYGSSPRTFGQGTKLEIK | | | | | | |
| | HbnC3t1p2_ | 1839 | EIVLTQSPGTLSLSPGERATLSCRASQS | IGHV1-58 | IGHJ3 | IGKV3-20 | IGKJ1 | 2868 | AAPYCSSTRC |
| | C6 | | VSSSYLAWYQQKPGQAPRLLIYGASSR | (Human) | (Human) | (Human) | (Human) | | YDAFDI |
| | | | ATGIPDRFSGSGSGTDFTLTISRLEPED | | | | | | |
| | | | FAVYYCQQYGRSPWTFGQGTKVEI | | | | | | |
| | HbnC4t1p1_ | 1840 | DIQMTQSPSSLSASVGDRATITCRASQ | IGHV3-9 | IGHJ4 | IGKV1-39 | IGKJ4 | 2869 | AKDINYDSGG |
| | D5 | | SISSYLNWYQEKPGKAPKLLIYAASSLQ | (Human) | (Human) | (Human) | (Human) | | YHKNYFDY |
| | | | SGVPSRFSGSGSGTDFTLTISSLQPEDF | | | | | | |
| | | | ATYYCQQSYSNPLTFGGGTKVEIK | | | | | | |
| | Ju et al., | | 205 | Various | Various | Various | Various | | Various |
| | 2020 | | | | | | | | |
| | Kim et | | Various | Various | Various | Various | Various | | Various |
| | al., 2020 | | | | | | | | |
| | LR1 | | N/A | IGHV3-3 | IGHJ4 | N/A | N/A | 2870 | AAAEWGYE |
| | | | | (Alpaca) | (Alpaca) | | | | WPLYYASSW |
| | | | | | | | | | Y |
| | LR11 | | N/A | IGHV3-3 | IGHJ4 | N/A | N/A | 2871 | AAAYWGWD |
| | | | | (Alpaca) | (Alpaca) | | | | WPLNSQDYW |
| | LR15 | | N/A | IGHV3-3 | IGHJ4 | N/A | N/A | 2872 | AAADWGYN |
| | | | | (Alpaca) | (Alpaca) | | | | WPLIREEYEY |
| | LR16 | | N/A | IGHV3-3 | IGHJ4 | N/A | N/A | 2873 | AAADWGYNI |
| | | | | (Alpaca) | (Alpaca) | | | | PLNITDYWY |
| | LR2 | | N/A | IGHV3-3 | IGHJ4 | N/A | N/A | 2874 | AAAMNGYNE |
| | | | | (Alpaca) | (Alpaca) | | | | PLYSYDYEY |
| | LR3 | | N/A | IGHV3-3 | IGHJ4 | N/A | N/A | 2875 | AAASWGYEW |
| | | | | (Alpaca) | (Alpaca) | | | | PLVYDDYWY |
| | LR5 | | N/A | IGHV3-3 | IGHJ4 | N/A | N/A | 2876 | AAATWGYH |
| | | | | (Alpaca) | (Alpaca) | | | | WPLGAWDY |
| | | | | | | | | | WY |
| | LR6 | | N/A | IGHV3-3 | IGHJ4 | N/A | N/A | 2877 | AAATWGYSW |
| | | | | (Alpaca) | (Alpaca) | | | | PLEHDEYWY |
| | LR7 | | N/A | IGHV3-3 | IGHJ4 | N/A | N/A | 2878 | AAAFHGEQY |
| | | | | (Alpaca) | (Alpaca) | | | | PLYTNKYHY |
| | LR8 | | N/A | IGHV3-3 | IGHJ4 | N/A | N/A | 2879 | AAANYGANF |
| | | | | (Alpaca) | (Alpaca) | | | | PLQANTYFY |
| | mAb-1 | 1841 | QPVLTQPPSVSGAPGQRITISCTGSSS | IGHV3-21 | IGHJ4 | IGLV1-40 | IGU1 | 2880 | ARDFSGHTAV |
| | | | NIGAGYDVHWYQQLPGTAPKLLIYGS | (Human) | (Human) | (Human) | (Human) | | AGTGFEY |
| | | | SSRPSGVPDRFSGSKSGTSASLAITGLQ | | | | | | |
| | | | AEDEADYYCQSYDSSLSVLYVFGTGTK | | | | | | |
| | | | VTVL | | | | | | |
| | mAb-10 | 1842 | DIVLTQTPATLSVSPGEGATLSCRASQS | IGHV3-66 | IGHJ4 | IGKV3-15 | IGKJ4 | 2881 | VRASPPGGN |
| | | | VRSNLAWFQQRPGQVPRLLIYDASTR | (Human) | (Human) | (Human) | (Human) | | TGWPFFED |
| | | | ATGVPARFTGSGSGTYFTLTISSLQSED | | | | | | |
| | | | FAVYYCQQYNSWPPLTFGGGTKLEIK | | | | | | |
| | mAb-100 | 1843 | EIVMTQSPATLSVSPGETATLSCRASQ | IGHV3-30 | IGHJ4 | IGKV3-11 | IGKJ1 | 2882 | ARDNALQNA |
| | | | SVGRFMGWYQQKPGQAPRLLIFDAS | (Human) | (Human) | (Human) | (Human) | | QIGYLDY |
| | | | NRVTGVPDRFRGSGSGTDFILTINSLEP | | | | | | |
| | | | EDSASYYCQRRGDGYNFGQGTKVEIK | | | | | | |
| | mAb-101 | 1844 | ETTLTQSPLSLPVTLGQPASISCRSSQG | IGHV1-69 | IGHJ6 | IGKV2-30 | IGKJ1 | 2883 | VLDSDPYTAT |
| | | | LVHSDGNTYLNWFQQRPGQSPRRLIY | (Human) | (Human) | (Human) | (Human) | | FSHNHYWYA |
| | | | KVSNRDSGVPDRFSGSGSGTDFTLKIS | | | | | | MDV |
| | | | RVEAEDIGVYYCMQGTEWPRTFGQG | | | | | | |
| | | | TKVEIK | | | | | | |
| | mAb-102 | 1845 | DIRVTQSPLSLPVTLGQPASISCRSSQR | IGHV1-69 | IGHJ6 | IGKV2-30 | IGKJ3 | 2884 | ARDPSIHYTG |
| | | | IVHTDGNTYLNWFLQRPGQSPRRLIYK | (Human) | (Human) | (Human) | (Human) | | NHHWYDLDI |
| | | | VSNRDSGVPDRFSGSGSGTDFTLKISR | | | | | | |
| | | | VEAEDVGIYYCMQGTEWPRTFGQGT | | | | | | |
| | | | KVDIK | | | | | | |
| | mAb-103 | 1846 | EIVMTQSPSSLSASVGDRVTITCRAGH | IGHV7-4- | IGHJ4 | IGKV1-39 | IGKJ3 | 2885 | AREEHYDFSS |
| | | | TISTYLNWYQQKPGKAPKILISGASSLQ | 1 | (Human) | (Human) | (Human) | | GYFRPAY |
| | | | SGVPSRFSGSGSGTDFTLTIGSLQPEDF | (Human) | | | | | |
| | | | ATYYCQQSYSTPYTFGQGTKVDIK | | | | | | |
| | mAb-104 | 1847 | QPVLTQPASVSGSPGQSSTLSCSGTSS | IGHV4-34 | IGHJ4 | IGLV2-23 | IGU3 | 2886 | ARGMTSPVV |
| | | | DVGSYDLVSWYQQHPGKAPKLMIYE | (Human) | (Human) | (Human) | (Human) | | HLYSSGRPSR |
| | | | GTKRPSGVSDRFSGSTSGNTASLTISGL | | | | | | WFDF |
| | | | QAEDEANYYCCSYAGSGTWIFGGGTK | | | | | | |
| | | | LTVL | | | | | | |
| | mAb-105 | 1848 | EIVLTQSPGTLSLSPGDRVTLFCRASQN | IGHV4-4 | IGHJ6 | IGKV3-20 | IGKJ1 | 2887 | ARSFISFDSSG |
| | | | IANNHLAWYQQKPGQAPRVLIYGAST | (Human) | (Human) | (Human) | (Human) | | HPYYYYAMD |
| | | | TATDIPDRFSGRVSGTDFTLTISRLDPE | | | | | | V |
| | | | DFAVYYCHQYGSSPWTFGQGTKLEIK | | | | | | |
| | mAb-106 | 1849 | ETTLTQSPATLSVSPGETATLSCRASQS | IGHV3-30 | IGHJ4 | IGKV3-11 | IGKJ1 | 2888 | ARDNALQNA |
| | | | VGRFMGWYQQKPGQAPRLLIFDASN | (Human) | (Human) | (Human) | (Human) | | QIGYLDY |
| | | | RVTGVPDRFRGSGSGTDFILTINSLEPE | | | | | | |
| | | | DSASYYCQRRGDGYNFGQGTKVEIK | | | | | | |
| | mAb-107 | 1850 | DIRMTQSPLSLPVDLGQSASISCRSSQ | IGHV1-69 | IGHJ6 | IGKV2-30 | IGKJ2 | 2889 | ARDPSILNTG |
| | | | RVVHTNGNTYLHWFHQRPGQAPRRL | (Human) | (Human) | (Human) | (Human) | | NHHWYDLD |
| | | | IYKVSNRESGVPDRFSGSGSGTDFTLRI | | | | | | M |
| | | | SRVEAEDVGVYYCMQATDWPRTFGQ | | | | | | |
| | | | GTKLEIK | | | | | | |
| | mAb-108 | 1851 | DIVMTQSPLSLSVTLGQAASISCTCSQT | IGHV1-69 | IGHJ6 | IGKV2-30 | IGKJ1 | 2890 | ARDPTFLNSG |
| | | | AVHSDGNTYLNWFHQRPGQSPRRLIY | (Human) | (Human) | (Human) | (Human) | | NHFWYDVDI |
| | | | KVSNRDSGVPDRFSGSGSGTDFTLKIN | | | | | | |
| | | | RVEAEDVGIYYCMQTTDWPRTFGQG | | | | | | |
| | | | TKVEIK | | | | | | |
| | mAb-109 | 1852 | ETTLTQSPVTLPVTLGQPASISCTSSRW | IGHV1-69 | IGHJ4 | IGKV2-30 | IGKJ2 | 2891 | ARDASFPNTG |
| | | | LVHTNGNTYLNWFHQRPGQSPRRLIY | (Human) | (Human) | (Human) | (Human) | | NHFWYDFDL |
| | | | QVSNRDSGVPDRFSGSGSGTDFTLQIS | | | | | | |
| | | | RVEAEDVGVYYCMQGTEWPRTFGQ | | | | | | |
| | | | GTKLEIK | | | | | | |
| | mAb-11 | 1853 | EIVLTQSPSSLSASVGDRVTITCQASQD | IGHV3-30 | IGHJ4 | IGKV1-33 | IGKJ5 | 2892 | AKKGSPYCGV |
| | | | IRKCLNWYQHIPGKAPKLLIHDASSLES | (Human) | (Human) | (Human) | (Human) | | DCYKGYFDY |
| | | | GVPSRFSGSGSGTDFSFTINSLHPEDIA | | | | | | |
| | | | TYYCQQFEDLPITFGQGTRLEIK | | | | | | |
| | mAb-110 | 1854 | DIVMTQSPDSLAVSLGERATINCKSSQ | IGHV3-33 | IGHJ4 | IGKV4-1 | IGKJ1 | 2893 | ARVGSGRVY |
| | | | SVLYSSNNKNYLAWYQQKPGQPPKLLI | (Human) | (Human) | (Human) | (Human) | | |
| | | | YWASTRESGVPDRFSGSGSGTDFTLTI | | | | | | |
| | | | SSLQAEDVAVYYCQQYYSTPYTFGQGT | | | | | | |
| | | | KVEIK | | | | | | |
| | mAb-111 | 1855 | DIVVTQSPGTLSLSPGERAALSCRASQS | IGHV4-4 | IGHJ3 | IGKV3-20 | IGKJ1 | 2894 | ARVRIGASHH |
| | | | VGNNYLAWYQQKPGQAPRLLIYGATS | (Human) | (Human) | (Human) | (Human) | | NFWSGYYTD |
| | | | RATGIPDRFSGSGSGTDFTLTISRLEPE | | | | | | AFDI |
| | | | DFAVYYCQQYGTSPVYTFGQGTKVEIK | | | | | | |
| | mAb-112 | 1856 | QPVLTQPPSVSAAPGQKVTISCSGSSS | IGHV5-51 | IGHJ4 | IGLV1-51 | IGU3 | 2895 | ARAPLASCSG |
| | | | NIENNYVSWYQQLPGAAPKLLIYDNN | (Human) | (Human) | (Human) | (Human) | | GRCPTYNRFD |
| | | | KRPSGIPDRFSGSKSGTSATLGITGLQT | | | | | | L |
| | | | GDEADYYCGTWDFSLSAGVFGGGTKL | | | | | | |
| | | | TVL | | | | | | |
| | mAb-113 | 1857 | ETTLTQSPLSLPVTLGQPASISCRSSQG | IGHV1-69 | IGHJ6 | IGKV2-30 | IGKJ1 | 2896 | ASEYFDGRSY |
| | | | LVHTNGNLYLNWFHQRPGQSPRRLIY | (Human) | (Human) | (Human) | (Human) | | HSFCGLDV |
| | | | QVSNRDSGVPDRFSGSGSGTDFTLRIS | | | | | | |
| | | | RVEAEDVGIYYCMQGTEWPRTFGQG | | | | | | |
| | | | TKVEIK | | | | | | |
| | mAb-114 | 1858 | DIQLTQSPSTLSASVGDSVTITCRASQS | IGHV3-23 | IGHJ6 | IGKV1-5 | IGKJ4 | 2897 | AKGLSFYGSG |
| | | | ISSWLAWYQQKPGKAPKLLIYKASSLET | (Human) | (Human) | (Human) | (Human) | | SDAFDV |
| | | | GVPSRFSGSGSGTEFTLTISSLQPDDFA | | | | | | |
| | | | TYYCQQYKSPLSFGGGTKVEIK | | | | | | |
| | mAb-115 | 1859 | DIQVTQSPATLSLSPGERATLSCRASQS | IGHV1-2 | IGHJ6 | IGKV3-11 | IGKJ5 | 2898 | ARDLISVIRGL |
| | | | VSSYLAWYQQKPGQAPRLLIYDASKR | (Human) | (Human) | (Human) | (Human) | | GGGMDV |
| | | | ATGIPARFSGSGSGTDFTLTISSLEPED | | | | | | |
| | | | FAVYYCQQRSDWHPITFGQGTRLEIK | | | | | | |
| | mAb-116 | 1860 | DIQVTQSPSSLSASVGDRVTITCRASQS | IGHV1-2 | IGHJ4 | IGKV1-39 | IGKJ3 | 2899 | ARGGPLPWS |
| | | | ISSSLNWYQQKPGKAPTLLIYTASNLQS | (Human) | (Human) | (Human) | (Human) | | DLDIVGTFDY |
| | | | GVPSRFSGSGSGTDFTLTITSLQPEDFA | | | | | | |
| | | | TYYCQQSYSTPGFGPGTKVDIK | | | | | | |
| | mAb-117 | 1861 | EIVMTQSPATLSLSPGERATLSCRASQS | IGHV3-33 | IGHJ2 | IGKV3-11 | IGKJ3 | 2900 | VKDQSSGDRL |
| | | | VSIYLAWYQQKPGQAPRLLIYDASNRA | (Human) | (Human) | (Human) | (Human) | | LYLGYFDL |
| | | | TGVPARFSGSGSGTDFTLTINNLEPED | | | | | | |
| | | | FAIYYCQQRAKWPPRVTFGPGTKVDIK | | | | | | |
| | mAb-118 | 1862 | DIVLTQSPATLSLSPGERATLSCRASQS | IGHV3-33 | IGHJ2 | IGKV3-11 | IGKJ3 | 2901 | VKDQSSGDRL |
| | | | VSIYLAWYQQKPGQAPRLLIYDASNRA | (Human) | (Human) | (Human) | (Human) | | LYLGYFDL |
| | | | TGIPARFSGSGSGTDFTLTINNLEPEDF | | | | | | |
| | | | AIYYCQQRAKWPPRVIFGPGTKVEIK | | | | | | |
| | mAb-119 | 1863 | SYELTQPPSVSVSPGQTARITCSGDALP | IGHV4-4 | IGHJ6 | IGLV3-10 | IGU3 | 2902 | ATMWGGLCT |
| | | | RRYAYWYQQRSGQAPVLVIYEDNKRP | (Human) | (Human) | (Human) | (Human) | | ASNCYGNPM |
| | | | SGIPERFSAFSSGTMATLTISGAQVEDE | | | | | | DV |
| | | | ADYYCYSTDSTANYKVFGGGTKLTVL | | | | | | |
| | mAb-12 | 1864 | ETTLTQSPLSLPVTLGQPASISCRSSQG | IGHV1-69 | IGHJ6 | IGKV2-30 | IGKJ3 | 2903 | VRDSDPYTAT |
| | | | LVHSNGNTYVNWFHQRPGQSPRRLIY | (Human) | (Human) | (Human) | (Human) | | VTSNHYWYA |
| | | | EVSNRDSGVPDRFSGSGSGTDFTLKIS | | | | | | MDV |
| | | | RVEAEDIGVYYCMQGTEWPRTFGQG | | | | | | |
| | | | TKVDIK | | | | | | |
| | mAb-120 | 1865 | QSVLIQPASVSGSPGQSITISCTGSSSD | IGHV4-34 | IGHJ5 | IGLV2-23 | IGU3 | 2904 | ARGQGGYDL |
| | | | VGSYNLVSWYQQHPGKAPKLMIYEGY | (Human) | (Human) | (Human) | (Human) | | RRVGYGLTS |
| | | | KRPSGVSNRFSGSKSGNTASLTISGLQ | | | | | | WFDP |
| | | | AEDEADYYCCSYAGSSAVVVVFGGGT | | | | | | |
| | | | KLTVL | | | | | | |
| | mAb-121 | 1866 | DIRVTQSPDSLAVSLGERATINCRTSQS | IGHV1-46 | IGHJ3 | IGKV4-1 | IGKJ3 | 2905 | ARVLAGSSHE |
| | | | VLYSSNNKNYLGWYQQKPGQPPKLLI | (Human) | (Human) | (Human) | (Human) | | WQLTHDAFD |
| | | | YWASTRESGVPDRFSGSGSGTDFTLTI | | | | | | |
| | | | SSLQAEDVAVYYCQQYYSTPYTFGQGT | | | | | | |
| | | | KVDIK | | | | | | |
| | mAb-122 | 1867 | EIVLTQSPATLSLSPGERATLSCRASQS | IGHV1-2 | IGHJ6 | IGKV3-11 | IGKJ5 | 2906 | AKDLITVIRGL |
| | | | VSTYLAWYQQKPGQAPRLLIYDASNR | (Human) | (Human) | (Human) | (Human) | | GGGMDV |
| | | | ATGIPARFSGSGSGTDFTLTISSLEPED | | | | | | |
| | | | FALYYCLQRSDWHPITFGQGTRLEIK | | | | | | |
| | mAb-123 | 1868 | DIQLTQSPATLSLSPGERATLSCRANQS | IGHV1-69 | IGHJ1 | IGKV3-11 | IGKJ4 | 2907 | ARDGPYDSG |
| | | | VSNFLAWYQQKPGQAPRHLIYDASNR | (Human) | (Human) | (Human) | (Human) | | GYHLNH |
| | | | ATGIPARFSGSGSGTDFTLTISSLEPED | | | | | | |
| | | | FAVYYCQQRSNWPPRLTFGGGTKVEIK | | | | | | |
| | mAb-124 | 1869 | DIVMTQTPGTLSVSPGERATLSCRASQ | IGHV3-11 | IGHJ4 | IGKV3-20 | IGKJ3 | 2908 | ARMGPYGSG |
| | | | IINRSQLGWYQHKPGQPPRLLIFDSSK | (Human) | (Human) | (Human) | (Human) | | TFDY |
| | | | RATGTPDRFSASGSETDFTLTISGVEPE | | | | | | |
| | | | DSGVYYCLQYSLATTFGPGTKVEIK | | | | | | |
| | mAb-125 | 1870 | QPVLTQSSSASASLGSSVKLTCTLSSGH | IGHV1-69 | IGHJ4 | IGLV4-60 | IGU6 | 2909 | ARDLSTLQPD |
| | | | SSYILAWHQQQPGKAPRFLMKFEVG | (Human) | (Human) | (Human) | (Human) | | AIVNFDY |
| | | | GRYNKGSGVPDRFSGSSSGADRYLTIS | | | | | | |
| | | | NLQSEDEADYYCETWDSNLKGVFGG | | | | | | |
| | | | GTKVTVL | | | | | | |
| | mAb-126 | 1871 | QPVLTQSSSASASLGSSVKLTCTLRSGH | IGHV1-69 | IGHJ4 | IGLV4-60 | IGU6 | 2910 | ARDLYYDNG |
| | | | SSYIIAWHQQQPGKAPRFLMKVGHSG | (Human) | (Human) | (Human) | (Human) | | GYNYLDY |
| | | | SYNKGSGVPDRFSGSRSGADHYLTISN | | | | | | |
| | | | LQPDDEADYYCEAWDNNNLGVFSGG | | | | | | |
| | | | TKVTVL | | | | | | |
| | mAb-127 | 1872 | DIQLTQSPSAMSASVGDRVTITCRASQ | IGHV3-48 | IGHJ6 | IGKV1-17 | IGKJ4 | 2911 | VRDYCNSVSC |
| | | | GINDNLAWFQQKPGKVPKRLIYAASN | (Human) | (Human) | (Human) | (Human) | | YTYYYIGMDV |
| | | | LQNGVPSRFSGSGSGTEFTLTISSLQPE | | | | | | |
| | | | DFATYYCLQHNSYPLTFGGGTKLEIK | | | | | | |
| | mAb-128 | 1873 | DIRMTQSPLSLPVTPGEPASISCRSSQS | IGHV1-18 | IGHJ6 | IGKV2-28 | IGKJ3 | 2912 | ATSASSYSRYY |
| | | | LLHSNGYNYLDWYLQKPGQSPQLLIYL | (Human) | (Human) | (Human) | (Human) | | FGLDV |
| | | | GSNRASGVPDRFSGSGSGTDFTLKISR | | | | | | |
| | | | VEAEDVGVYYCMQALQTPGVTFGGG | | | | | | |
| | | | TKVDIK | | | | | | |
| | mAb-129 | 1874 | QPVLTQPPSASGTPGQRVTISCSGSYS | IGHV4-31 | IGHJ6 | IGLV1-44 | IGU3 | 2913 | ARDLAKWSY |
| | | | NIGTNPANWYQQLPGTAPKLLIYNND | (Human) | (Human) | (Human) | (Human) | | GYYYSGMDV |
| | | | QRPSGVPDRFSGSKSGTSASLAISGLQ | | | | | | |
| | | | SEDETDYYCATWDDSLNGVVFGGGTK | | | | | | |
| | | | LTVL | | | | | | |
| | mAb-13 | 1875 | DIRMTQSPLSLPVTRGQPASISCRSSH | IGHV1-69 | IGHJ6 | IGKV2-30 | IGKJ4 | 2914 | AREEYSGTVH |
| | | | NVVHSDGKTYLNWFHQRPGQAPRRL | (Human) | (Human) | (Human) | (Human) | | NFFGMDV |
| | | | IYQVSKRDSGVPDRFSGSGSGSDFTLTI | | | | | | |
| | | | SRVEAEDVGVYYCMQGTDWPRSFGG | | | | | | |
| | | | GTKVEIK | | | | | | |
| | mAb-130 | 1876 | EIVLTQSPSSLSASVGDRVTITCRASRS | IGHV3-9 | IGHJ6 | IGKV1-39 | IGKJ1 | 2915 | AKDGRYCSGI |
| | | | ISSYLNWYQQKPGKAPNLLIYDASTLQS | (Human) | (Human) | (Human) | (Human) | | SCRTGMDV |
| | | | GVPSRFSGSGSGTDFSLTISSLQPEDFA | | | | | | |
| | | | TYYCLHTYTTPRTFGQGTKVEIK | | | | | | |
| | mAb-131 | 1877 | EIVLTQSPATLSVSPGERITLSCRASHS | IGHV3-48 | IGHJ3 | IGKV3-15 | IGKJ4 | 2916 | ARGLLDYLHD |
| | | | VSSNLAWYQQKPGQVPRLLIYGASARA | (Human) | (Human) | (Human) | (Human) | | AFDI |
| | | | TGIPARFSGSGSGTEFTLTISSLQSEDF | | | | | | |
| | | | AVYYCQQYNYWPPLTFGGGTKVEIK | | | | | | |
| | mAb-132 | 1878 | DIQMTQSPSSLSASVGDRVTITCRASQ | IGHV4-38 | IGHJ3 | IGKV1-39 | IGKJ5 | 2917 | ASRHLDLLPIG |
| | | | SISTYLNWYQQKTGKAPELLIYVASSLQ | 2 | (Human) | (Human) | (Human) | | SFDV |
| | | | SGVPSRFSGSGSGTDFTLTISSLQPEDF | (Human) | | | | | |
| | | | ATYYCQQSYRFPITFGQGTRLEIK | | | | | | |
| | mAb-133 | 1879 | EIVLTQSPATLSLSPGERATLSCRASQS | IGHV3-49 | IGHJ6 | IGKV3-11 | IGKJ3 | 2918 | TRGSGMFYG |
| | | | VGTYLAWYQQKHGQAPRLLISDVSKR | (Human) | (Human) | (Human) | (Human) | | SSSGMDV |
| | | | ATGIPARFSGSGSGTDFTLTITSLEPED | | | | | | |
| | | | FAVYYCQQRTNWPGATFGPGTKVDIK | | | | | | |
| | mAb-134 | 1880 | QPVLTQSPSASASLGGSVKLTCTLTSG | IGHV1-69 | IGHJ4 | IGLV4-69 | IGU3 | 2919 | AIRRDYSDYR |
| | | | HSTYAIAWHQQQPEKGPRFLMKLNS | (Human) | (Human) | (Human) | (Human) | | DFDY |
| | | | DGSHNKGDGIPDRFSGSSSGAERYLTI | | | | | | |
| | | | SSLQSEDEADYYCHTWGTDIQVFGGG | | | | | | |
| | | | TKLTVL | | | | | | |
| | mAb-135 | 1881 | NFMLTQPHSVSESPGKTVTISCTRSSG | IGHV4-59 | IGHJ4 | IGLV6-57 | IGU6 | 2920 | ARQSSSWYN |
| | | | SIANNYVQWLQQRPGSSPTTIIYEDNQ | (Human) | (Human) | (Human) | (Human) | | PYYFDQ |
| | | | RPSGVPDRFSGSIDASSNSASLTISGLK | | | | | | |
| | | | TEDEADYYCQSYDSSSQVFGGGTKVT | | | | | | |
| | | | VL | | | | | | |
| | mAb-136 | 1882 | DIVLTQSPATLSVSPGERITLSCRASHS | IGHV3-48 | IGHJ3 | IGKV3-15 | IGKJ4 | 2921 | ARGLLDYLHD |
| | | | VSSNLAWYQQKPGQVPRLLIYGASAR | (Human) | (Human) | (Human) | (Human) | | AFDI |
| | | | ATGIPARFSGSGSGTEFTLTISSLQSED | | | | | | |
| | | | FAVYYCQQYNYWPPLTFGGGTKVEIK | | | | | | |
| | mAb-137 | 1883 | ETTLTQSPLSLSVTLGQAASISCRPNLG | IGHV1-69 | IGHJ4 | IGKV2-30 | IGKJ2 | 2922 | ARDPSILNTG |
| | | | LMHTDGNTYLNWFHQRPGQSPRRLI | (Human) | (Human) | (Human) | (Human) | | NHHWYDLDL |
| | | | YKVSNRDSGVPDRFAGSGSGTHFTLEI | | | | | | |
| | | | SGVEADDVGVYYCMQGTEWPRTFG | | | | | | |
| | | | QGTKLEIK | | | | | | |
| | mAb-138 | 1884 | DIRMTQSPSTLSASVGDRVTITCRASQ | IGHV3-30 | IGHJ4 | IGKV1-5 | IGKJ2 | 2923 | ARPRSGSYRQ |
| | | | TIGSWLAWYQQKPGKAPNLLIYKASSL | (Human) | (Human) | (Human) | (Human) | | AIDY |
| | | | ESGVPSRFSGSGSGTEFTLTISSLQPDD | | | | | | |
| | | | FATYYCQQYNSLYTFGQGTKLEIK | | | | | | |
| | mAb-139 | 1885 | ETTLTQSPGTLSVSPGERATLSCRASQS | IGHV3-7 | IGHJ6 | IGKV3-15 | IGKJ3 | 2924 | VRQNVAIQYY |
| | | | VISNLAWYQQKPGQAPRLLIYGASTRA | (Human) | (Human) | (Human) | (Human) | | YYAMDV |
| | | | TGVPARFSGSGSGTEFTLTIASLQSEDF | | | | | | |
| | | | AVYYCQQYHHWPPYTFGQGTKVDIK | | | | | | |
| | mAb-14 | 1886 | EIVLTQSPDSLAVSLGERATINCKSSQS | IGHV3-30 | IGHJ4 | IGKV4-1 | IGKJ3 | 2925 | ARAKGGSYSN |
| | | | VLYSSNNENCLAWYQQKPGQPPKLLI | (Human) | (Human) | (Human) | (Human) | | AFDY |
| | | | YWASTRESGVPDRFSGSGSGTDFTLTI | | | | | | |
| | | | SSLQAEDVAVYYCQQYCSTPPYTFGQ | | | | | | |
| | | | GTKVDIK | | | | | | |
| | mAb-140 | 1887 | EIVMTQSPLSLPVSLGQPASISCRSSQS | IGHV1-69 | IGHJ3 | IGKV2-30 | IGKJ1 | 2926 | ARDPSILDTG |
| | | | VIHTDGNTYLNWYHQRPGQSPRRLIY | (Human) | (Human) | (Human) | (Human) | | NHHWYDLDI |
| | | | KVSNRDSGVPDRFSGSGSVTDFTLKIS | | | | | | |
| | | | RVEAEDVGVYYCMQGTEWPRTFGQ | | | | | | |
| | | | GTKVEIK | | | | | | |
| | mAb-141 | 1888 | DIVMTQSPLSLPVSRGQSASISCRSSHS | IGHV1-69 | IGHJ6 | IGKV2-30 | IGKJ3 | 2927 | AREEYSGTVH |
| | | | VVHSDGKTYVNWFHQRPGQAPRRLI | (Human) | (Human) | (Human) | (Human) | | NFFGMDV |
| | | | YQVSKRDSGVPDRFSGSGSGFDFTLKI | | | | | | |
| | | | SRVEAEDVGVYFCMQGTDWPRSFGG | | | | | | |
| | | | GTKVDIK | | | | | | |
| | mAb-142 | 1889 | DIVMTQSPSSLSASVGDRVTITCRASQ | IGHV3- | IGHJ3 | IGKV1-39 | IGKJ5 | 2928 | VRDWGSSTH |
| | | | SINIYLNWYQQKPGKAPKLLIYAASSLQ | 64D | (Human) | (Human) | (Human) | | YDVFDL |
| | | | SGVPSRFSGSGSGTDFTLTISSLQPEDF | (Human) | | | | | |
| | | | ATYYCQQSYSTPLITFGQGTRLEIK | | | | | | |
| | mAb-143 | 1890 | DIRLTQSPLSLPVTLGQAASISCKSSHF | IGHV1-69 | IGHJ3 | IGKV2-30 | IGKJ3 | 2929 | ARDPSILNTG |
| | | | IVHTDGNTYLNWFHQRPGQSPRRLIYK | (Human) | (Human) | (Human) | (Human) | | NHHWYDLDL |
| | | | VSNRDSGVPDRFSGSGSGTDFTLEISG | | | | | | |
| | | | VEAEDVGVYYCMQGTEWPRTFGQGT | | | | | | |
| | | | KVDIK | | | | | | |
| | mAb-144 | 1891 | ETTLTQSPLFLPVTLGQPASISCTSSVH | IGHV1-69 | IGHJ6 | IGKV2-30 | IGKJ3 | 2930 | ARDASIVGTG |
| | | | VVHSDGNTYLNWFQQRPGQSPRRLIY | (Human) | (Human) | (Human) | (Human) | | NHLWYGLDF |
| | | | KVSNRDSGVPDRFSGSGSGTYFTLKIS | | | | | | |
| | | | RVEAEDVGVYYCMQGTDWPRTFGQ | | | | | | |
| | | | GTKVDIK | | | | | | |
| | mAb-145 | 1892 | ETTLTQSPATLSLSPGERATLSCRASQS | IGHV3-30 | IGHJ5 | IGKV3-11 | IGKJ3 | 2931 | ARDNALQDG |
| | | | INDYLGWYQHRPGQAPRLLIHDASTRA | (Human) | (Human) | (Human) | (Human) | | RPGYFDS |
| | | | PGIPVRFSGSGSGTDFTLTISSLEPEDS | | | | | | |
| | | | AVYYCQQRFSWYNFGPGTKVEIK | | | | | | |
| | mAb-146 | 1893 | QSVLTQPASVSGSPGQSITISCAVTSSD | IGHV3-11 | IGHJ5 | IGLV2-23 | IGU3 | 2932 | ARGHRFLEFP |
| | | | VGSYNLVSWHQQHPGKAPKLMIYEV | (Human) | (Human) | (Human) | (Human) | | LNYFDP |
| | | | NKRPSGVSNRFSGSKSGNTASLTISGL | | | | | | |
| | | | QAEDEAVYYCCSYGGRSTSVVFGGGT | | | | | | |
| | | | KLTVL | | | | | | |
| | mAb-147 | 1894 | DIRLTQSPATLSLSPGERATLSCRASQS | IGHV3-30 | IGHJ5 | IGKV3-11 | IGKJ2 | 2933 | ARDNALQDG |
| | | | INDYLGWYQHRPGQAPRLLIHDASTRA | (Human) | (Human) | (Human) | (Human) | | RPGYFDS |
| | | | PGIPVRFSGSGSGTDFTLTISSLEPEDS | | | | | | |
| | | | AVYYCQQRFSWYNFGPGTKLEIK | | | | | | |
| | mAb-148 | 1895 | QPVLTQPASVSGSPGQSITISCTGTTSD | IGHV3-30 | IGHJ6 | IGLV2-14 | IGU3 | 2934 | ALLYGSGSYY |
| | | | VGGYDYVSWYQQRPGKAPKLIIYDVI | (Human) | (Human) | (Human) | (Human) | | NFVFFGWKD |
| | | | NRPSGVSNRFSGSKSGNTASLTISGLQ | | | | | | GSDA |
| | | | ADDETDYYCSSYTSGGTLVFGTGTKLT | | | | | | |
| | | | VL | | | | | | |
| | mAb-149 | 1896 | DIQMTQSPLSLPVGLGQSASISCRSSQ | IGHV1-69 | IGHJ4 | IGKV2-30 | IGKJ1 | 2935 | ARDPSILNTG |
| | | | WVVHTDGNTYLNWFHQRPGQSPRR | (Human) | (Human) | (Human) | (Human) | | NHHWYDLDL |
| | | | LIYKVSNRDSGVPDRFSGSGSGTDFTL | | | | | | |
| | | | RISRVEAEDVGVYYCMQATEWPRTFG | | | | | | |
| | | | QGTKVEIK | | | | | | |
| | mAb-15 | 1897 | DIRLTQSPLSLPVTLGQPASISCRSSQR | IGHV1-69 | IGHJ3 | IGKV2-30 | IGKJ3 | 2936 | ARDPSIHYTG |
| | | | IVHTDGNTYLNWFLQRPGQSPRRLIYK | (Human) | (Human) | (Human) | (Human) | | NHHWYDLDI |
| | | | VSNRDSGVPDRFSGSGSGTDFTLKISR | | | | | | |
| | | | VEAEDVGIYYCMQGTEWPRTFGQGT | | | | | | |
| | | | KVDIK | | | | | | |
| | mAb-150 | 1898 | DIQLTQSPLSLPVTLGQPASISCRSSQR | IGHV1-69 | IGHJ3 | IGKV2-30 | IGKJ1 | 2937 | ARDPSIHYTG |
| | | | IVHTDGNTYLNWFLQRPGQSPRRLIYK | (Human) | (Human) | (Human) | (Human) | | NHHWYDLDI |
| | | | VSNRDSGVPDRFSGSGSGTDFTLKISR | | | | | | |
| | | | VEAEDVGIYYCMQGTEWPRTFGQGT | | | | | | |
| | | | KVEIK | | | | | | |
| | mAb-151 | 1899 | ETTLTQSPLSLSVTLGQSASISCRASQT | IGHV1-69 | IGHJ6 | IGKV2-30 | IGKJ1 | 2938 | VRDSEPYTAT |
| | | | VVHSVDGNTYLNWFHQRPGQSPRRLI | (Human) | (Human) | (Human) | (Human) | | RSQNHYWYD |
| | | | YKVSNRDSGVPDRFSGSGSGTDFTLRI | | | | | | MDV |
| | | | SRVEAEDIGIYYCMQGTDWPRTFGQG | | | | | | |
| | | | TKVEIK | | | | | | |
| | mAb-152 | 1900 | DIVMTQSPLSLPVTLGQPASISCRSSQR | IGHV1-69 | IGHJ6 | IGKV2-30 | IGKJ1 | 2939 | ARDPSIHYTG |
| | | | IVHTDGNTYLNWFLQRPGQSPRRLIYK | (Human) | (Human) | (Human) | (Human) | | NHHWYDLDI |
| | | | VSNRDSGVPDRFSGSGSGTDFTLKISR | | | | | | |
| | | | VEAEDVGIYYCMQGTEWPRTFGQGT | | | | | | |
| | | | KVEIK | | | | | | |
| | mAb-153 | 1901 | DIRMTQSPLSLPVTLGQPASISCRSSQF | IGHV1-69 | IGHJ3 | IGKV2-30 | IGKJ3 | 2940 | ARDPSIVDSG |
| | | | VVHTDGNTYLNWFQQRPGQSPRRLIY | (Human) | (Human) | (Human) | (Human) | | PHHWYDLDI |
| | | | KVSNRDSGVPDRFSGSGSGTDFTLKIS | | | | | | |
| | | | RVEAEDVGVYYCMQATEWPRTFGQG | | | | | | |
| | | | TKVDIK | | | | | | |
| | mAb-154 | 1902 | SYVLTQPPSVSVSPGQTARITCSADGL | IGHV1-2 | IGHJ4 | IGLV3-25 | IGU7 | 2941 | ASGPNYFDY |
| | | | PKQYSYWYQQKPGQAPVMVIYKDTE | (Human) | (Human) | (Human) | (Human) | | |
| | | | RPSGIPERFSGSSSGTTATLTISGVQAE | | | | | | |
| | | | DEADYYCQSADSNDSSPVFGGGTQLT | | | | | | |
| | | | VL | | | | | | |
| | mAb-155 | 1903 | ETTLTQSPATLSLSPGEGATLSCRASQS | IGHV3-21 | IGHJ4 | IGKV3-11 | IGKJ1 | 2942 | ARADRDYDF |
| | | | VSSSLAWYQQKPGQAPRLLIYDASNR | (Human) | (Human) | (Human) | (Human) | | WSDPPLIDH |
| | | | ATGIPARFSGSGSGTDYTLTISSLEPED | | | | | | |
| | | | FAVYYCHQRSNWPYTFGQGTKVEIK | | | | | | |
| | mAb-156 | 1904 | DIVLTQTPSSLSASVGDRVTITCRASQT | IGHV3-53 | IGHJ4 | IGKV1-39 | IGKJ1 | 2943 | ARGFGNGWS |
| | | | IATYLSWYQQKPGKAPKLLIYAVSSLQS | (Human) | (Human) | (Human) | (Human) | | YYFDY |
| | | | GVPSRFSGSGSGTDFTLTIGSLQPEDF | | | | | | |
| | | | ATYYCQQSYSIPWTFGQGTKVDIK | | | | | | |
| | mAb-157 | 1905 | EIVMTQSPSSLSASVGDRVTITCRASQS | IGHV3-48 | IGHJ6 | IGKV1-39 | IGKJ4 | 2944 | ARDPGLEYSG |
| | | | ISYYLNWYQQKPGKAPKLLIYAASSLQS | (Human) | (Human) | (Human) | (Human) | | NYFSYYYYAM |
| | | | GVPSRFSGSGSGTDFILTISSLQPEDIA | | | | | | DV |
| | | | TYYCQHSYSSPPLTFGGGTKVEIK | | | | | | |
| | mAb-158 | 1906 | ETTLTQSPLSLPVTLGQPASISCRSSQI | IGHV1-69 | IGHJ6 | IGKV2-30 | IGKJ3 | 2945 | VRDSDPYTAT |
| | | | VVHSDGNTYLNWFHQRPGQSPRRLIYK | (Human) | (Human) | (Human) | (Human) | | SRNNHYWYD |
| | | | VSNRDSGVPDRFSGSGSGTDFTLKISR | | | | | | MDV |
| | | | VEAEDIGVYYCMQGTDWPRTFGQGT | | | | | | |
| | | | KVDIK | | | | | | |
| | mAb-159 | 1907 | DIRLTQSPLSLSVTLGQAASISCTCSQT | IGHV1-69 | IGHJ6 | IGKV2-30 | IGKJ1 | 2946 | ARDPTFLNSG |
| | | | AVHSDGNTYLNWFHQRPGQSPRRLIY | (Human) | (Human) | (Human) | (Human) | | NHFWYAVDI |
| | | | KVSNRDSGVPDRFSGSGSGTDFTLKIS | | | | | | |
| | | | RVEAEDVGIYYCMQTTDWPRTFGQG | | | | | | |
| | | | TKVEIK | | | | | | |
| | mAb-16 | 1908 | EIVLTQSPLSLPVTLGQPASISCRSSLR | IGHV1-69 | IGHJ6 | IGKV2-30 | IGKJ3 | 2947 | VRDSDPYTAT |
| | | | LVHTDGNTYLNWFQQRPGQSPRRLIYK | (Human) | (Human) | (Human) | (Human) | | YRNNHYWYA |
| | | | VSNRDSGVPDRFSGSGSGTDFTLKISR | | | | | | MDV |
| | | | VEAEDIGVYYCMQGTEWPRTFGQGT | | | | | | |
| | | | KVDIK | | | | | | |
| | mAb-160 | 1909 | DIVMTQSPGTLSLSPGERAALSCRASQ | IGHV3-11 | IGHJ4 | IGKV3-20 | IGKJ3 | 2948 | ARMGPSGSG |
| | | | IVTRSQLAWYQHKPGQPPRLLIYDSSS | (Human) | (Human) | (Human) | (Human) | | SLDY |
| | | | RATGTPDRFSGSGSGTDFTLTISRLEPE | | | | | | |
| | | | DSAVYYCHQYSGSATFGPGTKVEIK | | | | | | |
| | mAb-161 | 1910 | SYELTQPPSVSGAPRQKVTISCSGSSAN | IGHV2-5 | IGHJ4 | IGLV1-36 | IGU3 | 2949 | AHTSELPPRR |
| | | | IASNGVNWYQQLPGKAPKLLIYYDDL | (Human) | (Human) | (Human) | (Human) | | PYAAFDF |
| | | | VSSGVSDRFSGSKSGTSASLAISGLQSE | | | | | | |
| | | | DEADYYCATWDDILNGPVFGGGTKLT | | | | | | |
| | | | VL | | | | | | |
| | mAb-162 | 1911 | DIRMTQSPSSLSASVGDRVTITCRASQ | IGHV3-30 | IGHJ4 | IGKV1-27 | IGKJ1 | 2950 | ARDLPPLDY |
| | | | GFGNKVAWYQQKPGTAPKLLIYETSTL | (Human) | (Human) | (Human) | (Human) | | |
| | | | QSGVPSRFSGSGSGTEFAFTISSLQPED | | | | | | |
| | | | GATYYCQKYNRAPWTFGQGTKVEIK | | | | | | |
| | mAb-163 | 1912 | QSALIQPASVSGSPGQSITISCTGTSSD | IGHV4-34 | IGHJ4 | IGLV2-14 | IGU3 | 2951 | ARGFTFTYSD |
| | | | VGRYNYVSWYQQHPGKAPKLMIYDV | (Human) | (Human) | (Human) | (Human) | | FLTGQRTFEY |
| | | | SNRPSGVSNRFSGSKSGNTASLTISGL | | | | | | |
| | | | QAEDEAAYYCSSYISDIKLVVFGGGTKL | | | | | | |
| | | | TVL | | | | | | |
| | mAb-164 | 1913 | QSALIQPRSVSGSPGQSVTISCTGTSSD | IGHV3-30 | IGHJ4 | IGLV2-11 | IGU1 | 2952 | ARSYGGSYST |
| | | | VGGSNYVSWYQQHPGKAPKLLVYDV | (Human) | (Human) | (Human) | (Human) | | VGY |
| | | | TKRPSGVPDRFSGSKSGNTASLTISGL | | | | | | |
| | | | QAEDEADYYCCSYAGTYIFGTGTKLTV | | | | | | |
| | | | L | | | | | | |
| | mAb-165 | 1914 | QSVLTQPPSVSGSPGQSVSISCSGTSS | IGHV3-15 | IGHJ4 | IGLV2-18 | IGU1 | 2953 | SWNDVGWA |
| | | | DFGNYNRISWYQQTPGTAPKVIIYEVN | (Human) | (Human) | (Human) | (Human) | | FTF |
| | | | SRPSGVPDRFSGSKSGNTASLTITGLQ | | | | | | |
| | | | AEDEADYYCCSYRSDNTYIFGGGTKVT | | | | | | |
| | | | VL | | | | | | |
| | mAb-166 | 1915 | ETTLTQSPLFLPVTLGQPASISCRSSQR | IGHV1-69 | IGHJ6 | IGKV2-30 | IGKJ4 | 2954 | AAEERSGTNH |
| | | | LVHTNGNTYLNWFQQRPGHSPRRLIY | (Human) | (Human) | (Human) | (Human) | | NYYGLDV |
| | | | QVSNRDSGVPDRFSGSGSGTDFTLKIS | | | | | | |
| | | | RVEAEDVGVYYCMQGTEWPRTFGG | | | | | | |
| | | | GTKLEIK | | | | | | |
| | mAb-167 | 1916 | QPVLTQPPSASGTPGQRVTISCSGGSS | IGHV3-53 | IGHJ3 | IGLV1-44 | IGU1 | 2955 | ARDREMAHT |
| | | | NIGSNSVNWYQQLPGTAPKLLIYSNSQ | (Human) | (Human) | (Human) | (Human) | | ERSYGLDV |
| | | | RPSGVPDRFSGSKSGTSASLAISGLQSE | | | | | | |
| | | | DEADYYCAAWDDSLNTFRYVFGTGTK | | | | | | |
| | | | VTVL | | | | | | |
| | mAb-168 | 1917 | ETTLTQSPSSLSASVGDRVNITCRASQS | IGHV3-23 | IGHJ3 | IGKV1-39 | IGKJ3 | 2956 | AKDRYCSGGS |
| | | | ISTYLTWYQQKPGKAPKLLIYGASSLHS | (Human) | (Human) | (Human) | (Human) | | CFYDAFDI |
| | | | GVPSRFTGVGSGTEFTLSISSLQPEDFA | | | | | | |
| | | | TYYCQQSYNTFFTFGGGTKVDIK | | | | | | |
| | mAb-169 | 1918 | DIRLTQSPSSLSASVGDRVTITCRASQA | IGHV3-30 | IGHJ4 | IGKV1D-16 | IGKJ5 | 2957 | AREAQSSGRA |
| | | | IAGWLAWYQQKPGRAPKSLIYRASSL | (Human) | (Human) | (Human) | (Human) | | GCLDA |
| | | | QSGVPSRFSGSGSGTDFSLTISNLQPE | | | | | | |
| | | | DSATYYCQHYDSYPTAFGQGTRLEIK | | | | | | |
| | mAb-17 | 1919 | ETGLTQIPFSLPVTFGQPASISCRFSQR | IGHV1-69 | IGHJ3 | IGKV2-30 | IGKJ1 | 2958 | ARDPSIHYTG |
| | | | LVHTDGNTNLNWFLQRPGQFPRGLIY | (Human) | (Human) | (Human) | (Human) | | NHHWYDLDI |
| | | | KVFNRDSGVPDRFRGSGSGIDFTLKIS | | | | | | |
| | | | RVEVEDVGIYYGMQGTEWLGTFGQG | | | | | | |
| | | | TKVEIK | | | | | | |
| | mAb-170 | 1920 | ETTLTQSPLSLAVTLGQPASISCRSSLG | IGHV1-69 | IGHJ6 | IGKV2-30 | IGKJ1 | 2959 | VRDSDPYTAT |
| | | | LVHTNGNTYLNWFQQRPGQSPRRLIYR | (Human) | (Human) | (Human) | (Human) | | VRSNHYWYA |
| | | | VSNRDSGVPDRFSGSGSVTDFTLTISR | | | | | | MDV |
| | | | VEAEDIGVYYCMQGTEWPRTFGQGT | | | | | | |
| | | | KVEIK | | | | | | |
| | mAb-171 | 1921 | ETTLTQSPLSLSVTLGQAASISCTCSQT | IGHV1-69 | IGHJ6 | IGKV2-30 | IGKJ3 | 2960 | ARDPTFLNSG |
| | | | AVHSDGNTYLNWFHQRPGQSPRRLIY | (Human) | (Human) | (Human) | (Human) | | NHFWYAVDI |
| | | | KVSNRDSGVPDRFSGSGSGTDFTLKIS | | | | | | |
| | | | RVEAEDVGIYYCMQTTDWPRTFGQG | | | | | | |
| | | | TKVDIK | | | | | | |
| | mAb-172 | 1922 | EIVMTQSPASLSASVGDRVTITCRAGQ | IGHV4-39 | IGHJ5 | IGKV1-39 | IGKJ3 | 2961 | AAPAPSNHES |
| | | | SISTNLCWYQQRQGKAPKLLIYAASSL | (Human) | (Human) | (Human) | (Human) | | WSGTDWFDP |
| | | | RSGVPSRFSGSGSGTDFTLTISSLQPED | | | | | | |
| | | | FATYYCQQSYSTPPTFGGGTKVDIK | | | | | | |
| | mAb-173 | 1923 | DIQVTQSPATLSVSPGERVTLSCRASQ | IGHV1-18 | IGHJ4 | IGKV3-15 | IGKJ4 | 2962 | ARTTPRGWE |
| | | | SISNTLAWYQQKPGQAPRLLIYGASTR | (Human) | (Human) | (Human) | (Human) | | QWPVLEY |
| | | | ATGIPARFSGSGSGTEFTLTISSLQSED | | | | | | |
| | | | FAVYYCHQYNKWPPITFGGGTKVEIK | | | | | | |
| | mAb-174 | 1924 | DIQMTQSPSSLSASVGDRVTISCRASQ | IGHV3-48 | IGHJ6 | IGKV1-16 | IGKJ2 | 2963 | ARELDSETYY |
| | | | GISTFLAWFQQRPGKAPKSLIYAASKL | (Human) | (Human) | (Human) | (Human) | | NYNSLDV |
| | | | QSGVPSRFSGSDSGPDFTLTIDNLRPE | | | | | | |
| | | | DSATYYCKQYNSYPYTFGQGTKLEIK | | | | | | |
| | mAb-175 | 1925 | EIVMTQSPSSLSASVGDSVTITCRASQS | IGHV1-18 | IGHJ3 | IGKV1-39 | IGKJ1 | 2964 | ARVVFRHGQ |
| | | | ISNYLNWYQDKPGKAPELLIYAASNLQ | (Human) | (Human) | (Human) | (Human) | | YDDSSGRLAF |
| | | | SGVPSRFSGSGSGTDFTLTISSLQPEDF | | | | | | DI |
| | | | ATYYCQQSYSDSWTFGQGTKVDIK | | | | | | |
| | mAb-176 | 1926 | ETTLTQSPLSLPVTLGQPASISCRSSQR | IGHV1-69 | IGHJ3 | IGKV2-30 | IGKJ1 | 2965 | ARDPSIHYTG |
| | | | IVHTDGNTYLNWFLQRPGQSPRRLIYK | (Human) | (Human) | (Human) | (Human) | | NHHWYDLDI |
| | | | VSNRDSGVPDRFSGSGSGTDFTLKISR | | | | | | |
| | | | VEAEDVGIYYCMQGTEWPRTFGQGT | | | | | | |
| | | | KVEIK | | | | | | |
| | mAb-177 | 1927 | DIQMTQSPLSLPVTLGQPASISCRSSQS | IGHV1-69 | IGHJ6 | IGKV2-30 | IGKJ3 | 2966 | VRDSDPYTAT |
| | | | VVHSDGNTYLNWFHQRPGQSPRRLIY | (Human) | (Human) | (Human) | (Human) | | SRNNHYWYG |
| | | | KVSNRDSGVPDRFSGSGSGTDFTLKIS | | | | | | MDV |
| | | | RVEAEDIGVYYCMQGTDWPRTFGQG | | | | | | |
| | | | TKVDIK | | | | | | |
| | mAb-178 | 1928 | GIVLTQSPDSLAVSVGERATINCKSSQT | IGHV3-30 | IGHJ4 | IGKV4-1 | IGKJ3 | 2967 | AREPDGIGAA |
| | | | VLYSSKNKHYLAWYQQKPGQPPKLLT | (Human) | (Human) | (Human) | (Human) | | GISGY |
| | | | SSPSTREPGVPDRFSGSGSGTDFTLTIS | | | | | | |
| | | | SLQAEDVAVYYCQQYYTTPYTFGQGT | | | | | | |
| | | | KVDIK | | | | | | |
| | mAb-179 | 1929 | EIVMTQTPSSLSASVGDRVTITCRASQ | IGHV3-11 | IGHJ6 | IGKV1-39 | IGKJ3 | 2968 | AREMATSFGY |
| | | | SISNYVNWYQQKPGRAPNLLIYAASSL | (Human) | (Human) | (Human) | (Human) | | YFVLDV |
| | | | QSGVSSRFSGSGSGTDFTLTISSLQPED | | | | | | |
| | | | FATYYCQQTYSTPPEGPTFGPGTKVEIK | | | | | | |
| | mAb-18 | 1930 | QPVLTQPASVSGSPGQSVTISCTGTSS | IGHV4-34 | IGHJ5 | IGLV2-23 | IGU3 | 2969 | ARGQESPIVG |
| | | | DVGSYSDVGNYVSWYQHHPGKAPKL | (Human) | (Human) | (Human) | (Human) | | VTGRWFDP |
| | | | MIYEVRTRPSWVSTRFSGSKSGTTASL | | | | | | |
| | | | TISGLQAEDEADYYCCSYAGASPFVVF | | | | | | |
| | | | GGGTKLTVL | | | | | | |
| | mAb-180 | 1931 | EIVMTQSPGTLSSSPGERATLSCRASQ | IGHV3-30 | IGHJ4 | IGKV3-20 | IGKJ2 | 2970 | VRPYSGSYTN |
| | | | SVSSRYLAWYQQKPGQAPRLLIYGTSN | (Human) | (Human) | (Human) | (Human) | | WFDL |
| | | | RATGIPDRFSGSGSGTDFTLTISRLEPE | | | | | | |
| | | | DFAVYYCQQYGSSYTFGPGTKLEIK | | | | | | |
| | mAb-181 | 1932 | DIVMTQSPLSLPVGLGQSASISCRSSQ | IGHV1-69 | IGHJ3 | IGKV2-30 | IGKJ1 | 2971 | ARDPSILNTG |
| | | | RVVHTDGNTYLNWFHQRPGQSPRRLI | (Human) | (Human) | (Human) | (Human) | | NHHWYDLDI |
| | | | YKVSNRDSGVPDRFSGSGSGTDFTLRI | | | | | | |
| | | | SRVEAEDVGVYYCMQATEWPRTFGQ | | | | | | |
| | | | GTKVEIK | | | | | | |
| | mAb-182 | 1933 | DIRLTQSPLSLSVTLGQAASISCTCSQS | IGHV1-69 | IGHJ6 | IGKV2-30 | IGKJ1 | 2972 | ARDPTFLNTG |
| | | | AVHSDGTTYFNWFHQRPGQSPRRLIYK | (Human) | (Human) | (Human) | (Human) | | NHFWYAVDI |
| | | | VSNRDSGVPDRFIGSGSGTHFTLKISR | | | | | | |
| | | | VEAEDVGVYYCMQTTDWPRTFGQGT | | | | | | |
| | | | KVEIK | | | | | | |
| | mAb-183 | 1934 | ETTLTQSPLSLPVTLGQPASISCRSSQS | IGHV1-69 | IGHJ6 | IGKV2-30 | IGKJ1 | 2973 | VRDSDPYTAT |
| | | | VVHSDGNTYLNWFHQRPGQSPRRLIY | (Human) | (Human) | (Human) | (Human) | | SRNNHYWYA |
| | | | KVSNRDSGVPDRFSGSGSGTDFTLKIS | | | | | | MDV |
| | | | RVEAEDIGVYYCMQGTDWPRTFGQG | | | | | | |
| | | | TKVEIK | | | | | | |
| | mAb-184 | 1935 | ETTLTQSPLSLPVTLGQPASISCTSSQT | IGHV1-69 | IGHJ4 | IGKV2-30 | IGKJ2 | 2974 | ARDPSFLNTG |
| | | | VVHTDRNTYLNWYHQRPGQSPRRLIYK | (Human) | (Human) | (Human) | (Human) | | NHFWYDFDL |
| | | | VSNRDSGVPDRFSGSGSGSHFTLKISR | | | | | | |
| | | | VEAEDVGVYYCMQGTEWPRTFGQGT | | | | | | |
| | | | KLEIK | | | | | | |
| | mAb-185 | 1936 | QPGLTQPASVSGSPGQSITISCTGTSG | IGHV4-34 | IGHJ4 | IGLV2-23 | IGU3 | 2975 | ARGMISPRIP |
| | | | DVGSDNLVSWYQRHPGKAPKLMIYE | (Human) | (Human) | (Human) | (Human) | | RTTRQRWFD |
| | | | GSKRPSGVSHRFSGSNSGNTASLTISG | | | | | | T |
| | | | LQAEDDADYYCCSYAGDDTVVFGGGT | | | | | | |
| | | | KLTVL | | | | | | |
| | mAb-186 | 1937 | DIVLTQSPGTLSLSPGERVTLSCRASQS | IGHV4-34 | IGHJ5 | IGKV3-20 | IGKJ1 | 2976 | ARGKAHRND |
| | | | ISSNYLAWYQQKPGQAPRLLISDASSRA | (Human) | (Human) | (Human) | (Human) | | FWSGYYPHW |
| | | | TGIPDRFSGSGSGADFTLIISRLEPEDF | | | | | | FDP |
| | | | AVYFCHQYGGSPTTFGQGTKVEIK | | | | | | |
| | mAb-187 | 1938 | ETTLTQSPLSLPVTLGQPASISCRSSQI | IGHV1-69 | IGHJ6 | IGKV2-30 | IGKJ1 | 2977 | ARDSDPYTAT |
| | | | AMHSDGNTYLSWFHQRPGQPPRRLIYK | (Human) | (Human) | (Human) | (Human) | | RRHNHYWYA |
| | | | ISNRDSGVPDRFSGSGSGTDFTLKISRV | | | | | | MDV |
| | | | EAEDIGTFYCMQGTDWPRTFGQGTK | | | | | | |
| | | | VEIK | | | | | | |
| | mAb-188 | 1939 | ETTLTQSPATLSLSPGERATLSCRASQS | IGHV4-59 | IGHJ3 | IGKV3-11 | IGKJ4 | 2978 | ARGLVVRYFD |
| | | | VSSYLAWYQQKPGQAPRLLIYDAFNR | (Human) | (Human) | (Human) | (Human) | | GFPSGPIIGAF |
| | | | ATGVPARFSGSGSGTDFTLTISSLEPED | | | | | | DI |
| | | | FAVYYCQQRTSTLTFGGGTKVDIK | | | | | | |
| | mAb-189 | 1940 | ETTLTQSPLSLPVTLGQAASISCRSSQA | IGHV1-69 | IGHJ6 | IGKV2-30 | IGKJ1 | 2979 | VRDPDPYTAT |
| | | | VVHSDGNTYLNWFQQRPGQPPRRLIY | (Human) | (Human) | (Human) | (Human) | | VRHNHYWH |
| | | | KVSNRDSGVPDRFSGSGSGTVFTLKIS | | | | | | GMDV |
| | | | RVEAEDIGVYYCMQGTEWPRTFGQG | | | | | | |
| | | | TKVEIK | | | | | | |
| | mAb-19 | 1941 | QSVLTQPPSVSAAPGQKVTISCSGSSS | IGHV4-34 | IGHJ5 | IGLV1-51 | IGU3 | 2980 | ARGSLSREYD |
| | | | NLGNNYVSWYQQLPGTAPKLLIYDNH | (Human) | (Human) | (Human) | (Human) | | FLTAPQNGP |
| | | | KRPSGIPDRFSGSKSGTSATLGITGLQT | | | | | | WFDS |
| | | | GDEADYYCETWDSSLSVVVFGGGTKV | | | | | | |
| | | | TVL | | | | | | |
| | mAb-190 | 1942 | QSVLTQPPSVSATPGQKVTISCSGSSS | IGHV4-39 | IGHJ4 | IGLV1-51 | IGU3 | 2981 | ARHSQKDIVLI |
| | | | NIGNNYVSWYQQLPGTAPKLLIYDNN | (Human) | (Human) | (Human) | (Human) | | PAAQSPIFDY |
| | | | KRPSGIPDRFSGSKSGTSATLGITGLQT | | | | | | |
| | | | GDEADYYCGTWDSRLSAVVFGGGTKL | | | | | | |
| | | | TVL | | | | | | |
| | mAb-191 | 1943 | DIRVTQSPATLSLSPGERATLSCRASEN | IGHV3- | IGHJ1 | IGKV3-11 | IGKJ3 | 2982 | VKDGGYYDSS |
| | | | IAHYLAWYQQKPGQAPRLVIYDASSR | 64D | (Human) | (Human) | (Human) | | GPGH |
| | | | ATGIPGRFSGSGAGTDFTLTINSLEPED | (Human) | | | | | |
| | | | FAVYYCQQRSNWPQNFGGGTKVDIK | | | | | | |
| | mAb-192 | 1944 | DIRLTQSPSSLSASVGDRVTITCRASQD | IGHV3-30 | IGHJ5 | IGKV1D-16 | IGKJ5 | 2983 | ARESRSSGRA |
| | | | ISSWLAWYQQKSGKAPKSLIYAASSLQ | (Human) | (Human) | (Human) | (Human) | | GCFDS |
| | | | NGVPSRFSGSRSGTDFTLTISSLQPEDL | | | | | | |
| | | | GTYYCQQYDSYPVTFGQGTRLEIK | | | | | | |
| | mAb-193 | 1945 | DIVLTQTPDSLAVSLGERATINCKSSQS | IGHV3-30 | IGHJ4 | IGKV4-1 | IGKJ1 | 2984 | ARAYGGNYQ |
| | | | VLYSSNNKNYLAWYQQKPGQPPKLLI | (Human) | (Human) | (Human) | (Human) | | NHFDH |
| | | | YWASTRESGVPDRFSGSGSGTDFTLTI | | | | | | |
| | | | SSLQAEDVAVYYCQQYYTMWTFGQG | | | | | | |
| | | | TKVEIK | | | | | | |
| | mAb-194 | 1946 | QSVLTQPPSVSAAPGQKVTISCSGSSS | IGHV4-34 | IGHJ5 | IGLV1-51 | IGU1 | 2985 | ARGTISPIVGV |
| | | | NIGNDYVSWYQQLPGTAPKLLIYENYK | (Human) | (Human) | (Human) | (Human) | | PTPVVPRRGR |
| | | | RPSGIPDRFSGSKSGTSATLDITGLQTG | | | | | | SWFDP |
| | | | DEADYYCGTWDSSLSVDNYVFGTGTK | | | | | | |
| | | | LTVL | | | | | | |
| | mAb-195 | 1947 | DIVLTQSPLSLPVTLGQPASISCRSSQS | IGHV1-69 | IGHJ6 | IGKV2-30 | IGKJ3 | 2986 | VRDSDPYTAT |
| | | | VVHSDGNTYLNWFHQRPGQSPRRLIY | (Human) | (Human) | (Human) | (Human) | | SRNNHYWYA |
| | | | KVSNRDSGVPDRFSGSGSGTDFTLKIS | | | | | | MDV |
| | | | RVEAEDIGVYYCMQGTDWPRTFGQG | | | | | | |
| | | | TKVDIK | | | | | | |
| | mAb-196 | 1948 | ETTLTQSPLSLPVTLGQPASISCRSSQV | IGHV1-69 | IGHJ6 | IGKV2-30 | IGKJ3 | 2987 | VRDSDPYTAT |
| | | | LVHSDGNTYLNWFQQRPGQSPRRLIY | (Human) | (Human) | (Human) | (Human) | | SSHNHYWYA |
| | | | KVSNRDSGVPDRFSGSGSGIDFTLKISR | | | | | | MDV |
| | | | VEAEDIGVYYCMQGTEWPRTFGQGT | | | | | | |
| | | | KVDIK | | | | | | |
| | mAb-197 | 1949 | DIQLTQSPSSLSASVGDRVTITCRASQS | IGHV3-11 | IGHJ5 | IGKV1-39 | IGKJ4 | 2988 | ARDRGSGVID |
| | | | ISHYLNWYQQKPGKAPKLLIYAASNLQ | (Human) | (Human) | (Human) | (Human) | | P |
| | | | SGVPSRFSGNGSGTDFTLTISSLQPEDF | | | | | | |
| | | | ATYFCQQSYSTPLTFGGGTKVDIK | | | | | | |
| | mAb-2 | 1950 | EIVLTQSPATLSLSPGERATLSCRASEN | IGHV3- | IGHJ1 | IGKV3-11 | IGKJ4 | 2989 | VKDGGYYDSS |
| | | | IAHYLAWYQQKPGQAPRLVIYDASSRA | 64D | (Human) | (Human) | (Human) | | GPGH |
| | | | TGIPGRFSGSGAGTDFTLTINSLEPEDF | (Human) | | | | | |
| | | | AVYYCQQRSNWPQNFGGGTKVEIK | | | | | | |
| | mAb-20 | 1951 | QSVLTQPPSVSGAPGQRVTISCTGSSS | IGHV4-59 | IGHJ2 | IGLV1-40 | IGU6 | 2990 | ARVGWVRYF |
| | | | NIGAGYYVHWYQQLPGTAPKLLIYDN | (Human) | (Human) | (Human) | (Human) | | DWSKPYYYFD |
| | | | NNRPSGVPDRFSGSRSGTSASLAITGL | | | | | | L |
| | | | QAEDEGDYFCQSYDSSLSASFGGGTK | | | | | | |
| | | | VTVL | | | | | | |
| | mAb-21 | 1952 | QSVLTQPPSVSGAPGQRVTISCTGSNS | IGHV3-23 | IGHJ5 | IGLV1-40 | IGU3 | 2991 | AKCDLVRYFD |
| | | | NIGAGYDVHWYQQLPETAPKLLIYAN | (Human) | (Human) | (Human) | (Human) | | WLGEENNWF |
| | | | GNRPSGVPDRFSGSKSGTSASLAITGL | | | | | | DP |
| | | | QAEDEADYYCQSYDSSLSGVLFGGGT | | | | | | |
| | | | KLTVL | | | | | | |
| | mAb-22 | 1953 | DIQLTQSPLSLPVTLGQPASISCRSSQH | IGHV1-69 | IGHJ3 | IGKV2-30 | IGKJ1 | 2992 | VRDSDPYTAT |
| | | | LVHSDGNTYLNWFQQRPGQSPRRLIY | (Human) | (Human) | (Human) | (Human) | | VRNNHYWYA |
| | | | KVSNRDSGVPDRFSGSGSGIDFTLKISR | | | | | | LDV |
| | | | VEAEDIGVYYCMQGTEWPRTFGQGT | | | | | | |
| | | | KVEIK | | | | | | |
| | mAb-23 | 1954 | DIQVTQSPATVSVSPGEGASLSCRASQ | IGHV3-66 | IGHJ4 | IGKV3-15 | IGKJ4 | 2993 | ARATPPGGG |
| | | | SVRSNLAWFQQKPGQAPRLLISDAST | (Human) | (Human) | (Human) | (Human) | | TGWPYFDF |
| | | | RASGVPARFTGSGFGTEFTLTISSLQSE | | | | | | |
| | | | DFAIYYCQQYNTWPPLTFGGGTKVEIK | | | | | | |
| | mAb-24 | 1955 | DIRLTQSPLSLPVTLGQPASISCRSSQS | IGHV1-69 | IGHJ6 | IGKV2-30 | IGKJ4 | 2994 | AREEPSGTYH |
| | | | LVHSNGNTYLNWFQQRPGQSPRRLIYE | (Human) | (Human) | (Human) | (Human) | | NYYGLDV |
| | | | VSNRDSGVPDRFTGSGSGTDFTLKISR | | | | | | |
| | | | VEAEDVGVYYCMQGTHWPRSFGGG | | | | | | |
| | | | TKVEIK | | | | | | |
| | mAb-25 | 1956 | ETTLTQSPGTLSLSPGERATLSCRASQS | IGHV4-4 | IGHJ3 | IGKV3-20 | IGKJ1 | 2995 | AARIRGATHY |
| | | | IGSSYLAWYQQKPGQAPRLLIYGASSR | (Human) | (Human) | (Human) | (Human) | | DFWSGFWA |
| | | | ATGIPDRFSGSGSGTDFTLTISRLEPED | | | | | | GPFDI |
| | | | FAVYYCQQYGSAPLYTFGQGTKVEIK | | | | | | |
| | mAb-26 | 1957 | EIVLTQSPGTLSLSPGERASLSCRASQS | IGHV4-4 | IGHJ3 | IGKV3-20 | IGKJ3 | 2996 | ARTQSNDFW |
| | | | VSSSYLAWYQHRPGQAPRLLIYDASSR | (Human) | (Human) | (Human) | (Human) | | SGYYTAAFDL |
| | | | ATGIPDRFSGSGSGTDFTLTISRLESED | | | | | | |
| | | | FAVYYCQQYGNSPRTFGQGTKVDIK | | | | | | |
| | mAb-27 | 1958 | QPVLTQPPSVSAAPGQKVTISCSGSSS | IGHV4-34 | IGHJ5 | IGLV1-51 | IGU3 | 2997 | ARGSLSREYD |
| | | | NIGDNYVSWYQQLPGTAPKLLIYDNN | (Human) | (Human) | (Human) | (Human) | | FLTAPQNGP |
| | | | KRPSGIPDRFSGSKSGTSATLGITGLQT | | | | | | WFDS |
| | | | GDEADYYCETWDTSLSVVVFGGGTKL | | | | | | |
| | | | TVL | | | | | | |
| | mAb-28 | 1959 | DIVMTQTPLSLAVTLGQPASISCRSSH | IGHV1-69 | IGHJ6 | IGKV2-30 | IGKJ1 | 2998 | VLDTTSANPH |
| | | | GLVHTNGNTYLNWFHQRPGQPPRRL | (Human) | (Human) | (Human) | (Human) | | NWYGMDV |
| | | | IYKISNRDSGVPDRFSGSGSGTDFTLKI | | | | | | |
| | | | SRVEADDVGVYYCMQGTDWPRTFG | | | | | | |
| | | | QGTKVEIK | | | | | | |
| | mAb-29 | 1960 | EIVLTQSPGTLSLSPGERATLSCRASQT | IGHV3-11 | IGHJ4 | IGKV3-20 | IGKJ2 | 2999 | ARMGPYGSG |
| | | | VTRSQLAWYQHKPGQPPRLLIYDSSKR | (Human) | (Human) | (Human) | (Human) | | SFDY |
| | | | ATGSPDRFSASGSGTDFTLTISGLEPED | | | | | | |
| | | | TGIYYCLQYSDATTFGPGTKLEIK | | | | | | |
| | mAb-3 | 1961 | EIVMTQSPSSLSASIGDRVTISCRASQN | IGHV4-38 | IGHJ4 | IGKV1-39 | IGKJ5 | 3000 | AGRHQELLP |
| | | | IGSYLNWYQQRPGKAPNLLIFVASSLQ | 2 | (Human) | (Human) | (Human) | | MGSFDM |
| | | | SGVPSRFSGSGSGTDFTLTISSLQAEDF | (Human) | | | | | |
| | | | ATYYCQQSYTTPITFGQGTRLEIK | | | | | | |
| | mAb-30 | 1962 | DIVMTQSPLSLPVGLGQSASISCRSSQ | IGHV1-69 | IGHJ3 | IGKV2-30 | IGKJ1 | 3001 | ARDPSILNTG |
| | | | RVVHTDGNTYLNWFHQRPGQSPRRLI | (Human) | (Human) | (Human) | (Human) | | NHHWYDLDI |
| | | | YKVSNRDSGVPDRFSGSGSGTDFTLRI | | | | | | |
| | | | SRVEAEDVGVYYCMQATEWPRTFGQ | | | | | | |
| | | | GTKVEIK | | | | | | |
| | mAb-31 | 1963 | EIVLTQSPATLSLSPGERATLSCRASQS | IGHV1-69 | IGHJ4 | IGKV3-11 | IGKJ2 | 3002 | VRDGAYDSS |
| | | | VSNFLAWYQQKPGQPPRLLIYDASNR | (Human) | (Human) | (Human) | (Human) | | GYYSTQ |
| | | | ASGIAARFSGRGSGTDFTLTISSLEPED | | | | | | |
| | | | FAVYYCQQRRNWPPMYTFGQGTKLEI | | | | | | |
| | | | K | | | | | | |
| | mAb-32 | 1964 | EIVLTQSPLSLSVTLGQAASISCTCSQT | IGHV1-69 | IGHJ6 | IGKV2-30 | IGKJ2 | 3003 | ARDPTFLNSG |
| | | | AVHSDGNTYLNWFHQRPGQSPRRLIYK | (Human) | (Human) | (Human) | (Human) | | NHFWYAVDI |
| | | | VSNRDSGVPDRFSGSGSGTDFTLKISR | | | | | | |
| | | | VEAEDVGIYYCMQTTDWPRTFGQGT | | | | | | |
| | | | KLEIK | | | | | | |
| | mAb-33 | 1965 | ETTLTQSPLSLSVTLGQAASISCTCSQS | IGHV1-69 | IGHJ6 | IGKV2-30 | IGKJ3 | 3004 | ARDPTFLNTG |
| | | | AVHSDGNTYFNWFHQRPGQSPRRLIY | (Human) | (Human) | (Human) | (Human) | | NHFWYAVDI |
| | | | KVSNRDSGVPERFSGSGSGTHFTLIISR | | | | | | |
| | | | VEAEDVGVYYCMQTTDWPRTFGQGT | | | | | | |
| | | | KVDIK | | | | | | |
| | mAb-34 | 1966 | NFMLTQPHSVSESPGKAVIISCTRSSG | IGHV1-2 | IGHJ5 | IGLV6-57 | IGU3 | 3005 | ARDFGVRYD |
| | | | NIASNFVQWYQQRPGSSPTPVIYEDKL | (Human) | (Human) | (Human) | (Human) | | DSRQLMKYC |
| | | | RPSGVPDRFSGSIDRSSNSASLTISGLK | | | | | | DS |
| | | | TEDEADYYCQSYDSGNLVVFGGGTKL | | | | | | |
| | | | TVL | | | | | | |
| | mAb-35 | 1967 | DIQMTQSPLSLPVTLGQAASISCRSSQ | IGHV1-69 | IGHJ6 | IGKV2-30 | IGKJ1 | 3006 | ARDPSILNTG |
| | | | PIPHTDGNTYLNWFHQRPGQSPRRLI | (Human) | (Human) | (Human) | (Human) | | NHHWYDLDL |
| | | | HKVSNRDSGVPDRFSGSGSGLDFTLEI | | | | | | |
| | | | SGVEAEDVGIYYCMQGTEWPRTFGQ | | | | | | |
| | | | GTKVEIK | | | | | | |
| | mAb-36 | 1968 | EIVMTQTPLSLPVTLGQPASISCRSSHG | IGHV1-69 | IGHJ6 | IGKV2-30 | IGKJ3 | 3007 | VVDTTMADP |
| | | | LVHTNGNTYLNWFHQRPGQPPRRLIY | (Human) | (Human) | (Human) | (Human) | | HNWYGLDV |
| | | | QVSHRDSGVPDRFSGSGSGTDFTLKIS | | | | | | |
| | | | RVEADDVGVYYCMQGTDWPRTFGQ | | | | | | |
| | | | GTKVDIK | | | | | | |
| | mAb-37 | 1969 | ETTLTQSPGTLSLSPGERATLSCRASQS | IGHV4-4 | IGHJ3 | IGKV3-20 | IGKJ1 | 3008 | ARVRVGASH |
| | | | VGNNYLAWYQQKPGQAPRLLIYGASS | (Human) | (Human) | (Human) | (Human) | | HNFWSGYYT |
| | | | RATGIPDRFSGSGSGTDFTLTISRLEPE | | | | | | DAFDI |
| | | | DFAVYYCQQYGTSPVYTFGQGTKVEIK | | | | | | |
| | mAb-38 | 1970 | DIQLTQSPLSLPVTLGQPASISCTSSQN | IGHV1-69 | IGHJ4 | IGKV2-30 | IGKJ1 | 3009 | ARDPSFLNTG |
| | | | VVHTDGNTYLNWFHQRPGQSPRRLIY | (Human) | (Human) | (Human) | (Human) | | NHFWYDFD |
| | | | KVSNRDSGVPDRFSGSGSGSHFTLKIS | | | | | | M |
| | | | RVEAEDVGIYYCMQGTDWPRTFGQG | | | | | | |
| | | | TKVEIK | | | | | | |
| | mAb-39 | 1971 | DIVLTQTPATLSLSPGERATLSCRASQS | IGHV1-69 | IGHJ4 | IGKV3-11 | IGKJ1 | 3010 | VRDGAYDSS |
| | | | VSNFLAWYQQKPGQPPRLLIYDASNR | (Human) | (Human) | (Human) | (Human) | | GYYSTQ |
| | | | ASGIAARFSGRGSGTDFTLTISSLEPED | | | | | | |
| | | | FAVYYCQQRRNWPPMYTFGQGTKVE | | | | | | |
| | | | IK | | | | | | |
| | mAb-4 | 1972 | QSVLTQPPSVSGAPGQRVTISCTGSSS | IGHV3-21 | IGHJ4 | IGLV1-40 | IGU1 | 3011 | ARDFPGDTA |
| | | | NIGAGYDVHWYQQVPGTAPKLLIYGN | (Human) | (Human) | (Human) | (Human) | | VAGTGFNY |
| | | | SNRPSGVPDRFSGSKSGTSASLAITGL | | | | | | |
| | | | QAEDEADYYCQSYDSSLSVLYVFGTGT | | | | | | |
| | | | KVTVL | | | | | | |
| | mAb-40 | 1973 | DIRLTQSPATLSLSPGERATLSCRASQS | IGHV3-49 | IGHJ4 | IGKV3-11 | IGKJ1 | 3012 | SRDLRRGYYD |
| | | | IGYYLAWFQQKPGQAPRLLIYDASKRA | (Human) | (Human) | (Human) | (Human) | | SNGHQQFDL |
| | | | TGIPARFSGSGSGTDFTLTISSLEPEDF | | | | | | |
| | | | AVYYCQHRTNWPYTFGQGTKVEIK | | | | | | |
| | mAb-41 | 1974 | QPGLTQEPSLTVSPGGTVTLTCGSSAG | IGHV3-30 | IGHJ6 | IGLV7-46 | IGU3 | 3013 | ARDPGPITFF |
| | | | AVTSGHYPYWFQQKPGQAPRTLIYDT | (Human) | (Human) | (Human) | (Human) | | DWSPDKSRK |
| | | | NNKYSWTPARFSGSLLGGKAALTLSG | | | | | | SYYDYNGMD |
| | | | AQPEDEAEYFCFLSYRGAPPVFGGGT | | | | | | V |
| | | | HLTVL | | | | | | |
| | mAb-42 | 1975 | DIRMTQSPFSLSASVGDRVTITCRASQ | IGHV3-30 | IGHJ4 | IGKV1-27 | IGKJ1 | 3014 | ARDLPPLDY |
| | | | GFGNRLAWYQQKPGRAPKLLIYDAST | (Human) | (Human) | (Human) | (Human) | | |
| | | | LQSGVPSRFSGSGSETDFALTISSLQPE | | | | | | |
| | | | DVATYYCQKHDRDPWTFGQGTKVEIK | | | | | | |
| | mAb-43 | 1976 | DIVMTQSPLSLPVTLGQPASISCRSSQR | IGHV1-69 | IGHJ6 | IGKV2-30 | IGKJ1 | 3015 | VRDSDPYTTT |
| | | | LVHSDGNTYLNWFQQRPGQSPRRLIY | (Human) | (Human) | (Human) | (Human) | | FSHNHYWYA |
| | | | KVSNRDSGVPDRFSGSGSGTDFTLKIS | | | | | | MDV |
| | | | RVEAEDIGVYYCMQGTEWPRTFGQG | | | | | | |
| | | | TKVEIK | | | | | | |
| | mAb-44 | 1977 | ETTLTQSPASVSASVGDRVTISCRASQ | IGHV3- | IGHJ4 | IGKV1-12 | IGKJ4 | 3016 | VKDNVILPGA |
| | | | GIGRWLAWYQQKPGRAPKLLIFSASSL | 64D | (Human) | (Human) | (Human) | | IVRPQFDY |
| | | | QSGVPSRFSGSGSGTDFTLTISSLQPED | (Human) | | | | | |
| | | | FATYYCQQAESFPFTFGGGTKVEIK | | | | | | |
| | mAb-45 | 1978 | ETTLTQSPLSLPVDLGQSASISCRSSQR | IGHV1-69 | IGHJ6 | IGKV2-30 | IGKJ1 | 3017 | ARDPSILNTG |
| | | | VVHTNGNTYLHWFHQRPGQAPRRLI | (Human) | (Human) | (Human) | (Human) | | NHHWYDLD |
| | | | YKVSNRESGVPDRFSGSGSGTDFTLRI | | | | | | M |
| | | | SRVEAEDVGVYYCMQATDWPRTFGQ | | | | | | |
| | | | GTKVEIK | | | | | | |
| | mAb-46 | 1979 | CIRLTQSPATLSLSPGERATLSCRASQS | IGHV3-30 | IGHJ4 | IGKV3-11 | IGKJ3 | 3018 | ARDNVVQQN |
| | | | VTSYLAWYQQRPGQAPRLLIYDTSNR | (Human) | (Human) | (Human) | (Human) | | ADNVGYFDF |
| | | | VTGVPVRFSGSGYGTDFTLTISSLEPED | | | | | | |
| | | | FAVYYCQQRGNGYTFGPGTKVEIK | | | | | | |
| | mAb-47 | 1980 | ETTLTQSPLSLPVGLGQSASISCRSSQR | IGHV1-69 | IGHJ6 | IGKV2-30 | IGKJ3 | 3019 | ARDPSILNTG |
| | | | VVHTDGNTYLNWFHQRPGQSPRRLIY | (Human) | (Human) | (Human) | (Human) | | NHHWYDLDL |
| | | | KVSNRDSGVPDRFSGSGSGTDFTLRIS | | | | | | |
| | | | RVEAEDVGVYYCMQATEWPRTFGQG | | | | | | |
| | | | TKVDIK | | | | | | |
| | mAb-48 | 1981 | EIVMTQSPLSLPVTLGQPASISCRSSQS | IGHV1-69 | IGHJ6 | IGKV2-30 | IGKJ2 | 3020 | VRDSDPYTAT |
| | | | VVHSDGNTYLNWFHQRPGQSPRRLIY | (Human) | (Human) | (Human) | (Human) | | SRNNHYWYG |
| | | | KVSNRDSGVPDRFSGSGSGTDFTLKIS | | | | | | MDV |
| | | | RVEAEDIGVYYCMQGTDWPRTFGQG | | | | | | |
| | | | TKLEIK | | | | | | |
| | mAb-49 | 1982 | DIVMTQSPLSLPVDLGQSASISCRSSQT | IGHV1-69 | IGHJ3 | IGKV2-30 | IGKJ2 | 3021 | ARDPSILNTG |
| | | | AVHTNGNTYLHWFHQRPGQAPRRLI | (Human) | (Human) | (Human) | (Human) | | NHHWYDLD |
| | | | YKVSNRESGVPDRFSGSGSGTDFTLRI | | | | | | M |
| | | | NRVEAEDVGVYYCMQATDWPRTFG | | | | | | |
| | | | QGTKLEIK | | | | | | |
| | mAb-5 | 1983 | DIVLTQTPSSLSASVGDRVTITCRASQT | IGHV3-49 | IGHJ6 | IGKV1-39 | IGKJ3 | 3022 | AREGTSLGYY |
| | | | ISYYLNWYQQKVGKAPQLLVYAASSLQ | (Human) | (Human) | (Human) | (Human) | | YYYAMDV |
| | | | SGVPSRFSGSGSGTDFTLTISSLQPEDF | | | | | | |
| | | | ATYYCQQSYSVPLTLGQGTKVDIK | | | | | | |
| | mAb-50 | 1984 | DIQMTQSPLSLPVDLGQSASISCRSSQ | IGHV1-69 | IGHJ6 | IGKV2-30 | IGKJ1 | 3023 | ARDPSILNTG |
| | | | RVVHTNGNTYLHWFHQRPGQAPRRL | (Human) | (Human) | (Human) | (Human) | | NHHWYDLD |
| | | | YKVSNRESGVPDRFSGSGSGTDFTLRI | | | | | | M |
| | | | SRVEAEDVGVYYCMQATDWPRTFGQ | | | | | | |
| | | | GTKVEIK | | | | | | |
| | mAb-51 | 1985 | QPGLTQPASVSGSPGQSITISCTRTSG | IGHV3-7 | IGHJ3 | IGLV2-14 | IGU3 | 3024 | ARGPIRHFGL |
| | | | DVGDYNSVSWYVSWYQQHPGRAPKL | (Human) | (Human) | (Human) | (Human) | | DAFDI |
| | | | MLYDVSNRPSGVSNRFSGSKLGDTAS | | | | | | |
| | | | LTISELQAEDEADYYCSTYTSTSTIFGG | | | | | | |
| | | | GTKLTVL | | | | | | |
| | mAb-52 | 1986 | DIQMTQSPLSLPVTLGQPASISCRASQ | IGHV1-69 | IGHJ6 | IGKV2-30 | IGKJ3 | 3025 | VRDSDPYTAT |
| | | | TVVHTNGNTYLNWFHQRPGQSPRRLI | (Human) | (Human) | (Human) | (Human) | | SRNNHYWYG |
| | | | YEVSNRDSGVPDRFSGSGSGTDFTLSI | | | | | | MDV |
| | | | SRVEAEDIGVYYCMQGTDWPRTFGP | | | | | | |
| | | | GTKVEIK | | | | | | |
| | mAb-53 | 1987 | DIRLTQSPLSLPVDLGQSASISCRSSQT | IGHV1-69 | IGHJ3 | IGKV2-30 | IGKJ3 | 3026 | ARDPSILNTG |
| | | | VVHTNGNTYLHWFHQRPGQAPRRLI | (Human) | (Human) | (Human) | (Human) | | NHHWYDLD |
| | | | YKVSNRESGVPDRFSGSGSGTDFTLRI | | | | | | M |
| | | | SRVEAEDVGVYYCMQATDWPRTFGQ | | | | | | |
| | | | GTKVDIK | | | | | | |
| | mAb-54 | 1988 | DIRVTQSPSSLSATVGDRVTITCRASQT | IGHV3- | IGHJ3 | IGKV1-39 | IGKJ5 | 3027 | VKDLGATVTY |
| | | | ITKYLNWYQQKPGKAPKLLLYGASSLQ | 64D | (Human) | (Human) | (Human) | | DVFDV |
| | | | SGVPSRFSGSGSGTDFTLTISSLQPEDF | (Human) | | | | | |
| | | | ATYYCQQTYITPGTFGQGTRLEIK | | | | | | |
| | mAb-55 | 1989 | DIRLTQSPDSLALSLGERATINCKSSQS | IGHV3-23 | IGHJ4 | IGKV4-1 | IGKJ1 | 3028 | AKERDLPGRG |
| | | | VLFSSNNNNYLGWYQQKPGQPPKLLI | (Human) | (Human) | (Human) | (Human) | | GYFDH |
| | | | YWASTRESGVPDRFSGSGSGTDFTLTI | | | | | | |
| | | | SSLQAEDVAVYYCQQYYLTPWTFGQG | | | | | | |
| | | | TKVDIK | | | | | | |
| | mAb-56 | 1990 | QPVLTQPASVSGSPGQSITISCTGTSSD | IGHV4-34 | IGHJ5 | IGLV2-23 | IGU3 | 3029 | ARGYTAPIIRE |
| | | | VGNYNVVSWYQQHPGKAPQLFIYED | (Human) | (Human) | (Human) | (Human) | | VPITFRPRWF |
| | | | TKRPSGVSDRFSGSKSGITASLTISRLQ | | | | | | DP |
| | | | PEDEADYYCCSYAVSGTVLFGGGTKVT | | | | | | |
| | | | VL | | | | | | |
| | mAb-57 | 1991 | EIVLTQSPGTLSVSPGERATLSCRASQI | IGHV3-11 | IGHJ4 | IGKV3-20 | IGKJ3 | 3030 | ARMGPYGSG |
| | | | INRSQLGWYQHKPGQAPRLLIFDSSKR | (Human) | (Human) | (Human) | (Human) | | TFDY |
| | | | ATGTPDRFSASGSETDFTLTISGVEPED | | | | | | |
| | | | SGVYYCLQYSLATTFGPGTKVDIK | | | | | | |
| | mAb-58 | 1992 | DIQLTQSPATLSLSPGERATLSCRASPS | IGHV3-30 | IGHJ5 | IGKV3-11 | IGKJ3 | 3031 | ARDHALQNG |
| | | | VFTFLAWYQQRPGQPPRLLIHDVSNR | (Human) | (Human) | (Human) | (Human) | | RPGYFDS |
| | | | APGIPARFSGSGSGTDFTLIISSLEPDD | | | | | | |
| | | | SAVYFCQQRSDGYNFGPGTKVDIK | | | | | | |
| | mAb-59 | 1993 | DIRVTQSPSSLSASVGDRVTITCRASQT | IGHV3-64 | IGHJ4 | IGKV1-39 | IGKJ1 | 3032 | VREGQQWLG |
| | | | INNYLNWYQQKPGKAPNLLIYAASTLQ | (Human) | (Human) | (Human) | (Human) | | LYFDH |
| | | | NGVPSRFSGSGSGTDFTLTISSVQPED | | | | | | |
| | | | FATYYCQQTYITPTWTFGQGTKVEIK | | | | | | |
| | mAb-6 | 1994 | EIVMTQSPSSLSASVGDRVTITCRASQS | IGHV1-8 | IGHJ4 | IGKV1-39 | IGKJ3 | 3033 | AREKKSFGPQ |
| | | | ISNYLYWYQQKPGKAPKLLIYVASNLQ | (Human) | (Human) | (Human) | (Human) | | YYYGSGED |
| | | | SGVPSRFSGSGSGTDFTLTISSLQPEDF | | | | | | |
| | | | ATYYCQQSYSTPYNFGQGTKVDIK | | | | | | |
| | mAb-60 | 1995 | EIVLTQSPSTLSASVGDRVTITCRASQN | IGHV5-51 | IGHJ3 | IGKV1-5 | IGKJ3 | 3034 | VRQRYCSGGS |
| | | | VNNWLAWYQQKPGKAPKLLIYEASTL | (Human) | (Human) | (Human) | (Human) | | CFLFEDAFEI |
| | | | KSGVPSRFSGSGSGTEFTLTISSLQPDD | | | | | | |
| | | | FATYYCQQYNSQYTFGQGTKVDIK | | | | | | |
| | mAb-61 | 1996 | QPVLTQPRSVSGSPGQSVTISCTGSSST | IGHV3-48 | IGHJ5 | IGLV2-11 | IGU3 | 3035 | VRDTDWAFD |
| | | | FGTDNHISWYQQLPGKVPKLIIHAVSQ | (Human) | (Human) | (Human) | (Human) | | s |
| | | | RPSVVPDRFSGSRSGNTASLTISGLQA | | | | | | |
| | | | EDEADYYCCSSPGTITWVFGGGTKVT | | | | | | |
| | | | VL | | | | | | |
| | mAb-62 | 1997 | QSVLTQPPSVSGVPRQKVTISCSGSTA | IGHV2-5 | IGHJ4 | IGLV1-36 | IGU3 | 3036 | AHTSELPPRR |
| | | | NIASNGVNWYQLVPGKAPRLLISYDDL | (Human) | (Human) | (Human) | (Human) | | PYAAFDF |
| | | | VPSGVSARFSGSKSGTSASLAISGLQAE | | | | | | |
| | | | DEADYYCATWDDILNGPVFGGGTKLT | | | | | | |
| | | | VL | | | | | | |
| | mAb-63 | 1998 | DIVLTQSPLSLPVTLGQSASISCRSSQG | IGHV3-23 | IGHJ4 | IGKV2-30 | IGKJ2 | 3037 | ANTNFLDY |
| | | | LVHSDGNIYLNWFQQRPGQSPRRLIY | (Human) | (Human) | (Human) | (Human) | | |
| | | | KVSNRDSGVPDRFSGSGSGTDFTLKIS | | | | | | |
| | | | RVEAEDVGLYYCMQATHWPRAFGQ | | | | | | |
| | | | GTKLEIK | | | | | | |
| | mAb-64 | 1999 | EIVMTQSPGTLSLSPGERVTLSCRATQ | IGHV4-4 | IGHJ6 | IGKV3-20 | IGKJ1 | 3038 | TRSFISFDSSG |
| | | | SVSNNHLAWYQQKPGQAPRLLIYGAS | (Human) | (Human) | (Human) | (Human) | | HPYYYYAMD |
| | | | TTATDIPDRFSGRVAGTDFTLTISRLDP | | | | | | V |
| | | | EDFAVYYCHQYGSSPWTFGQGTKVEI | | | | | | |
| | | | K | | | | | | |
| | mAb-65 | 2000 | DIRLTQSPATLSLSPGERATLSCRASQS | IGHV1-69 | IGHJ4 | IGKV3-11 | IGKJ1 | 3039 | TRETGTDEFD |
| | | | VSSSLAWYQQKPGQAPRLLIYEASNRA | (Human) | (Human) | (Human) | (Human) | | F |
| | | | TGVPARFSGSGSGTDFTLAISSLEPEDF | | | | | | |
| | | | AVYYCQHRSNWPPRYTFGQGTKVEIK | | | | | | |
| | mAb-66 | 2001 | SYELTQPPSVSVSPGQTARITCSGDALP | IGHV4-4 | IGHJ6 | IGLV3-10 | IGU6 | 3040 | ATMWGGLCT |
| | | | RRYAYWYQQRSGQAPVLVIYEDNKRP | (Human) | (Human) | (Human) | (Human) | | ASNCYGNPM |
| | | | SGIPERFSAFSSGTRATLTISGAQVEDE | | | | | | DV |
| | | | ADYYCYSTDSTANYKVFGGGTKVTVL | | | | | | |
| | mAb-67 | 2002 | SYELTQPPSVSVSPGQTARITCSGDALP | IGHV4-4 | IGHJ6 | IGLV3-10 | IGU3 | 3041 | ATMWGGLCT |
| | | | RRYAYWYQQRSGQAPVLVIYEDNKRP | (Human) | (Human) | (Human) | (Human) | | ASNCYGNPM |
| | | | SGIPERFSAFSSGTRATLTISGAQVEDE | | | | | | DV |
| | | | ADYYCYSTDSTANYKVFGGGTKLTVL | | | | | | |
| | mAb-68 | 2003 | SYELTQPPSVSVSPGQTARITCSGDALP | IGHV4-4 | IGHJ6 | IGLV3-10 | IGU3 | 3042 | ATMWGGLCT |
| | | | RRYAYWYQQRSGQAPVLVIYEDNKRP | (Human) | (Human) | (Human) | (Human) | | ASNCYGNPM |
| | | | SGIPERFSAFSSGTRATLTISGAQVEDE | | | | | | DV |
| | | | ADYYCYSTDSTANYKVFGGGTKLTVL | | | | | | |
| | mAb-69 | 2004 | QSVLTQPSSLSASPGASASLTCTLHSGF | IGHV3-74 | IGHJ4 | IGLV5-45 | IGU3 | 3043 | ARDLAWTFF |
| | | | NVGDHTINWYQQRPGSPPRYLLKYKS | (Human) | (Human) | (Human) | (Human) | | DY |
| | | | DSDKEQGSGVPIRFSGSKDASANAGFL | | | | | | |
| | | | LISGLRSEDEADYYCMIWHDNAVVFG | | | | | | |
| | | | GGTKLTVL | | | | | | |
| | mAb-7 | 2005 | EIVLTQSPATLSVSPGDGASLSCRASQS | IGHV3-66 | IGHJ4 | IGKV3-15 | IGKJ4 | 3044 | ARATPPGGTT |
| | | | VGSNLAWYQQKPGQAPRLLISDASAR | (Human) | (Human) | (Human) | (Human) | | GWPYIDL |
| | | | ATGVPARFTGSGSGTDFTLTISSLQSED | | | | | | |
| | | | FAVYYCHQYNTWPPLTFGGGTKLEIK | | | | | | |
| | mAb-70 | 2006 | EIVMTQSPLSLPVTLGQSASISCRSSQG | IGHV3-23 | IGHJ4 | IGKV2-30 | IGKJ3 | 3045 | ANTNFLDY |
| | | | LVHSDGNIYLNWFQQRPGQSPRRLIY | (Human) | (Human) | (Human) | (Human) | | |
| | | | KVSNRDSGVPDRFSGSGSGTDFTLKIS | | | | | | |
| | | | RVEAEDVGLYYCMQATHWPRAFGQ | | | | | | |
| | | | GTKVDIK | | | | | | |
| | mAb-71 | 2007 | DIVMTQSPLALSVTPGQPASISCKSSQ | IGHV3-9 | IGHJ6 | IGKV2D-29 | IGKJ5 | 3046 | AKLGTDHPIG |
| | | | SLLHSDGKTYFYWYLQKPGQSPHLLIY | (Human) | (Human) | (Human) | (Human) | | VDV |
| | | | EVSNRFSGVPDRFSGSGSGTDFTLKISR | | | | | | |
| | | | VEAEDVGVYYCMQSIQVPITFGGGTK | | | | | | |
| | | | LEIK | | | | | | |
| | mAb-72 | 2008 | DIVMTQSPATLSVSPGERATLSCRASQ | IGHV3- | IGHJ4 | IGKV3-15 | IGKJ3 | 3047 | VKALYSSSWC |
| | | | SVSSNLAWYQQKPGQAPRLLIYGAST | 64D | (Human) | (Human) | (Human) | | PFDY |
| | | | RATNIPARFSGSGSGTEFTLTISSLQSE | (Human) | | | | | |
| | | | DFAVYYCQQYNLWPYTFGQGTKVDIK | | | | | | |
| | mAb-73 | 2009 | EIVLTQSPSSLSASVGDRVTISCRASQG | IGHV3-48 | IGHJ6 | IGKV1-16 | IGKJ2 | 3048 | ARELDSETYY |
| | | | ISTFLAWFQQRPGKAPKSLIYAASKLQS | (Human) | (Human) | (Human) | (Human) | | NYNSLDV |
| | | | GVPSRFSGSDSGPDFTLTIDNLQPEDS | | | | | | |
| | | | ATYYCKQYNSYPYTFGQGTKLEIK | | | | | | |
| | mAb-74 | 2010 | QSVVTQPPSVSAAPGQKVTISCSGSSS | IGHV4-59 | IGHJ5 | IGLV1-51 | IGU3 | 3049 | AKAQGIYYRG |
| | | | NIGNNYVSWYQQLPGTAPKLLIYDSD | (Human) | (Human) | (Human) | (Human) | | WSYWFDP |
| | | | KRPSGIPERFSGSKSATSATLGITGLQT | | | | | | |
| | | | GDEADYYCETWDDSLSAVVFGGGTKL | | | | | | |
| | | | TVL | | | | | | |
| | mAb-75 | 2011 | DIVLTQSPLSLSVTPGQPASISCRSSQS | IGHV1-46 | IGHJ5 | IGKV2D-29 | IGKJ5 | 3050 | AREARRQVT |
| | | | LQHTDGKTYLYWYLQKPGQSPQLLIYEL | (Human) | (Human) | (Human) | (Human) | | QWFGEFWG |
| | | | FNRFSGVPERFSGSGSGTDFTLKISRVE | | | | | | PYNWFDP |
| | | | AEDVGIYYCMQSIQTPITFGQGTRLEIK | | | | | | |
| | mAb-76 | 2012 | DIRVTQSPDSLAVSLGERATINCKSSQS | IGHV4-34 | IGHJ4 | IGKV4-1 | IGKJ4 | 3051 | ARGLISYTLW |
| | | | VLYSSNNKNYLAWYQQKIGQPPKLLIY | (Human) | (Human) | (Human) | (Human) | | LRESYFDY |
| | | | WASIRESGVPDRFTGSGSGTDFTLTISS | | | | | | |
| | | | LQAEDVAVYYCQQYYSTPPTFGGGTK | | | | | | |
| | | | LEIK | | | | | | |
| | mAb-77 | 2013 | EIVMTQTPLSLPVTLGQPASISCRSSQF | IGHV1-69 | IGHJ6 | IGKV2-30 | IGKJ1 | 3052 | ARDPSILNTG |
| | | | VSHTDGNTYLNWFQQRPGQSPRRLIY | (Human) | (Human) | (Human) | (Human) | | NHHWYDLDI |
| | | | KVSKRDSGVPDRFSGSGSGTDFTLTIS | | | | | | |
| | | | RVEAEDVGVYYCMQATEWPRTFGQG | | | | | | |
| | | | TKVEYQ | | | | | | |
| | mAb-78 | 2014 | DIVMTQSPDSLAVSLGERATINCKSSQ | IGHV1-18 | IGHJ4 | IGKV4-1 | IGKJ3 | 3053 | ARDYGDGPP |
| | | | SVLFSSNNKNYLAWYQQKPGLPPKLLI | (Human) | (Human) | (Human) | (Human) | | DH |
| | | | YWASTRKSGVSDRFSGSGSGTDFTLTI | | | | | | |
| | | | SSLQAEDVAVYYCQQYYTTPLFTFGPG | | | | | | |
| | | | TKVDIK | | | | | | |
| | mAb-79 | 2015 | DIVMTQSPLSLPVTLGQPASISCRSSHS | IGHV1-69 | IGHJ6 | IGKV2-30 | IGKJ3 | 3054 | VLDTTMSHP |
| | | | LVHTNGNTYLNWFQQRPGQPPRRLIY | (Human) | (Human) | (Human) | (Human) | | HNWYGMDV |
| | | | QVSNRDSGVPDRFSGSGSGTDFTLKIS | | | | | | |
| | | | RVEADDVGIYYCMQGTDWPRTFGQG | | | | | | |
| | | | TKVDIK | | | | | | |
| | mAb-8 | 2016 | ETTLTQSPLSLPVTLGQPASISCRSSQG | IGHV1-69 | IGHJ4 | IGKV2-30 | IGKJ1 | 3055 | ASEYFDGRSY |
| | | | LAHSNGNTYLNWFHQRPGQSPRRLIY | (Human) | (Human) | (Human) | (Human) | | HSFCGLDV |
| | | | QVSNRDSGVPDRFSGSGSGTDFTLKIS | | | | | | |
| | | | RVEAEDVGVYYCMQGTEWPRTFGQ | | | | | | |
| | | | GTKVEIK | | | | | | |
| | mAb-80 | 2017 | DIVMTQSPLSLPVSLGQPASISCRSSQS | IGHV1-69 | IGHJ6 | IGKV2-30 | IGKJ3 | 3056 | ARDPSILNTG |
| | | | VVHTDGNTYLNWYQQRPGQSPRRLIY | (Human) | (Human) | (Human) | (Human) | | PHHWYDLDI |
| | | | KVSNRDSGVPDRFSGSGSVTDFTLKIS | | | | | | |
| | | | RVEAEDVAVYYCMQGTEWPRTFGQG | | | | | | |
| | | | TKVDIK | | | | | | |
| | mAb-81 | 2018 | DIQMTQSPLSLSVTLGQSASISCRASQT | IGHV1-69 | IGHJ6 | IGKV2-30 | IGKJ1 | 3057 | VRDSEPYTAT |
| | | | VVHSVDGNTYLNWFHQRPGQSPRRLL | (Human) | (Human) | (Human) | (Human) | | RSQNHYWYD |
| | | | YKVSNRDSGVPDRFSGSGSGTDFTLRI | | | | | | MDV |
| | | | SRVEAEDIGIYYCMQGTDWPRTFGQG | | | | | | |
| | | | TKVEIK | | | | | | |
| | mAb-82 | 2019 | QPVLTQPASVSGSPGQSITISCTGTSSD | IGHV4-4 | IGHJ6 | IGLV2-14 | IGU1 | 3058 | AREFGVRFLD |
| | | | VGSFNYVSWYQQHPGKAPKLVIYDVY | (Human) | (Human) | (Human) | (Human) | | RSLFGAMDV |
| | | | NRPSGVSNRFSGSKSGNTASLTISGLQ | | | | | | |
| | | | AEDEADYYCSSYTSSSTLYVFGTGTKVT | | | | | | |
| | | | VL | | | | | | |
| | mAb-83 | 2020 | DIRLTQSPSTLSASVGDRVTITCRASQS | IGHV3-30 | IGHJ3 | IGKV1-5 | IGKJ1 | 3059 | ARAGGYLSAF |
| | | | ISSWLAWYQQKPGKAPNLLIYKASTLES | (Human) | (Human) | (Human) | (Human) | | DI |
| | | | GVPSRFSGSGSGTEFTLTISSLQPDDFA | | | | | | |
| | | | TYYCQQYNSGWTFGQGTKVEIK | | | | | | |
| | mAb-84 | 2021 | DIRLTQSPLSLPVTLGQPASISCTSSQD | IGHV1-69 | IGHJ3 | IGKV2-30 | IGKJ3 | 3060 | ARDPSFLNAG |
| | | | VVHTDGNTYLNWFHQRPGQSPRRLIY | (Human) | (Human) | (Human) | (Human) | | NHFYYDFDV |
| | | | KVSNRDSGVPDRFSGSGSGSHFTLKIS | | | | | | |
| | | | RVEAEDAGIYYCMQGTDWPRTFGQG | | | | | | |
| | | | TKVDIK | | | | | | |
| | mAb-85 | 2022 | EIVLTQSPSSLSASVGDRVTITCRASQD | IGHV4-4 | IGHJ4 | IGKV1-9 | IGKJ3 | 3061 | ARAGNIVVM |
| | | | ISNYLAWYQQKPGKAPKLLIYAASTLQS | (Human) | (Human) | (Human) | (Human) | | PAAQYYFDY |
| | | | GVPLRFSGSGSGTDFTLTISSLQPEDFA | | | | | | |
| | | | TYYCLQLHNYSSFGGGTKVDIK | | | | | | |
| | mAb-86 | 2023 | EIVLTQSPATLSLSPGERATLSCRASQS | IGHV3-30 | IGHJ5 | IGKV3-11 | IGKJ2 | 3062 | VRDDVLQHS |
| | | | VSTFLGWYQQRPGQPPRLLIYDASYR | (Human) | (Human) | (Human) | (Human) | | RPSGPGYFVS |
| | | | APDIPVRFSGSGSGTDFTLTINSLEPED | | | | | | |
| | | | SAVYYCQQRSDGYNFGPGTKLEIK | | | | | | |
| | mAb-87 | 2024 | QSVLTQPASVSGSPGQSITISCTGTSSD | IGHV4-59 | IGHJ4 | IGLV2-14 | IGU3 | 3063 | ARGSQIDLRG |
| | | | VGAYNYVSWYQQHPGKAPKLTVYDV | (Human) | (Human) | (Human) | (Human) | | GLGATFFDY |
| | | | RNRPSGVSNRFSGSKSGNTASLTISGL | | | | | | |
| | | | QAEDEAHYYCSSYTSTNTVFGGGTKLT | | | | | | |
| | | | VL | | | | | | |
| | mAb-88 | 2025 | DIRLTQTPLSLPVTLGQPASISCRSSQI | IGHV1-69 | IGHJ6 | IGKV2-24 | IGKJ3 | 3064 | ARDSDPYTAT |
| | | | AMHSDGNTYLSWFHQRPGQPPRRLIYK | (Human) | (Human) | (Human) | (Human) | | RRHNHYWYA |
| | | | ISNRDSGVPDRFSGSGSGTDFTLKISRV | | | | | | MDV |
| | | | EAEDIGTFYCMQGTDWPRTFGQGTK | | | | | | |
| | | | VDIK | | | | | | |
| | mAb-89 | 2026 | QSALIQPRSVSGSPGQSVTISCTGSSST | IGHV3-48 | IGHJ5 | IGLV2-11 | IGU3 | 3065 | VRDTDWAFD |
| | | | FGTDNHISWYQQLPGKVPKLIIHAVSQ | (Human) | (Human) | (Human) | (Human) | | s |
| | | | RPSVVPDRFSGSRSGNTASLTISGLQA | | | | | | |
| | | | EDEADYYCCSSPGTITWVFGGGTKVT | | | | | | |
| | | | VL | | | | | | |
| | mAb-9 | 2027 | DIRLTQSPSSLSASVGDRVTIACRASQS | IGHV1-8 | IGHJ6 | IGKV1-39 | IGKJ1 | 3066 | ARRGNNFGY |
| | | | VSNYLNWYQQKPGKAPKLLIYAASSLQ | (Human) | (Human) | (Human) | (Human) | | YYYYTVDV |
| | | | NGVPSRFGGSGSGTDFTLTISSLQPED | | | | | | |
| | | | FATYYCQQCYSYPPTFGHGTKVEIK | | | | | | |
| | mAb-90 | 2028 | DIRMTQSPDSLAVSLGERATINCRTSQ | IGHV1-46 | IGHJ3 | IGKV4-1 | IGKJ3 | 3067 | ARVLAGSSHE |
| | | | SVLYSSNNKNYLGWYQQKPGQPPKLL | (Human) | (Human) | (Human) | (Human) | | WQLTHDAFD |
| | | | IYWASTRESGVPDRFSGSGSGTDFTLTI | | | | | | |
| | | | SSLQAEDVAVYYCQQYYSTPYTFGQGT | | | | | | |
| | | | KVDIK | | | | | | |
| | mAb-91 | 2029 | QSALTQPASVSGSPGQSITISCTGTNSD | IGHV3-23 | IGHJ4 | IGLV2-14 | IGU3 | 3068 | AKGDWIRYF |
| | | | ISNSYSVSWYQQYPGKAPKLVIFDVIN | (Human) | (Human) | (Human) | (Human) | | DWSLPISFFD |
| | | | RPSGVSNRFSGSKSGNTASLTISGLQA | | | | | | Y |
| | | | DDEADYYCCSYTRTNTPVLFGGGTKLT | | | | | | |
| | | | VL | | | | | | |
| | mAb-92 | 2030 | DIQLTQSPATLSVSPGERATLSCRASQS | IGHV4-61 | IGHJ4 | IGKV3-15 | IGKJ4 | 3069 | ARTTSPLTYSG |
| | | | VTKHLAWYQQKPGQAPRLLIYGASTR | (Human) | (Human) | (Human) | (Human) | | HWPLFDY |
| | | | ATGVPARFSGSGSDTEFSLTISSLQSED | | | | | | |
| | | | FAVYYCQQYYSWPPLTFGGGTKLEIK | | | | | | |
| | mAb-93 | 2031 | DIVMTQTPLSLPVTLGQPASISCRASQ | IGHV1-69 | IGHJ6 | IGKV2-30 | IGKJ3 | 3070 | VRDSDPYTAT |
| | | | TVVHTNGNTYLNWFHQRPGQSPRRLI | (Human) | (Human) | (Human) | (Human) | | SRNNHYWYG |
| | | | YEVSNRDSGVPDRFSGSGSGTDFTLSI | | | | | | MDV |
| | | | SRVEAEDIGVYYCMQGTDWPRTFGP | | | | | | |
| | | | GTKVEIK | | | | | | |
| | mAb-94 | 2032 | DIQLTQSPLSLPVGLGQSASISCRSSQR | IGHV1-69 | IGHJ6 | IGKV2-30 | IGKJ2 | 3071 | ARDPSILNTG |
| | | | VVHTDGNTYLNWYHQRPGQSPRRLIY | (Human) | (Human) | (Human) | (Human) | | NHHWYDLDI |
| | | | KVSNRDSGVPDRFSGSGSGTDFTLRIS | | | | | | |
| | | | RVEAEDVGVYYCMQSTDWPRTFGQG | | | | | | |
| | | | TKLEIK | | | | | | |
| | mAb-95 | 2033 | DIVLTQSPLSLPVGLGQSASISCRSSQR | IGHV1-69 | IGHJ3 | IGKV2-30 | IGKJ2 | 3072 | ARDPSILNTG |
| | | | VVHTDGNTYLHWFHQRPGQSPRRLIY | (Human) | (Human) | (Human) | (Human) | | NHHWYDLDI |
| | | | KVSNRDSGVPDRFSGSGSGTDFTLRIS | | | | | | |
| | | | RVEAEDVGVYYCMQGTEWPRTFGQ | | | | | | |
| | | | GTKLEIK | | | | | | |
| | mAb-96 | 2034 | ETTLTQSPVTLSVSPGERATLSCRASQS | IGHV3-66 | IGHJ5 | IGKV3-15 | IGKJ4 | 3073 | VRMDWME |
| | | | VISNLAWYQQKPGQAPRLLIFGASTRA | (Human) | (Human) | (Human) | (Human) | | WMKYYFDS |
| | | | TGVPARFSGSGSGTEFTLTISSLQSEDF | | | | | | |
| | | | AVYYCQQYNNWPSLTFGGGTKLEIK | | | | | | |
| | mAb-97 | 2035 | DIQVTQSPLSLPVGLGQSASISCRSSQR | IGHV1-69 | IGHJ3 | IGKV2-30 | IGKJ3 | 3074 | ARDPSILNTG |
| | | | VVHTDGNTYLNWFHQRPGQSPRRLIY | (Human) | (Human) | (Human) | (Human) | | NHHWYDLD |
| | | | KVSNRDSGVPDRFSGSGSGTDFTLRIS | | | | | | M |
| | | | RVEAEDVGVYYCMQATEWPRTFGQG | | | | | | |
| | | | TKVDIK | | | | | | |
| | mAb-98 | 2036 | EIVMTQSPATLSLSPGERATLSCRASQS | IGHV3-30 | IGHJ5 | IGKV3-11 | IGKJ2 | 3075 | VRDDVLQHS |
| | | | VSTFLGWYQQRPGQPPRLLIYDASYR | (Human) | (Human) | (Human) | (Human) | | RPSGPGYFVS |
| | | | APDIPVRFSGSGSGTDFTLTINSLEPED | | | | | | |
| | | | SAVYYCQQRSDGYNFGPGTKLEIK | | | | | | |
| | mAb-99 | 2037 | QPVLTQPPSVSVSPGQTARITCSGDAL | IGHV4-4 | IGHJ6 | IGLV3-10 | IGU3 | 3076 | ATMWGGLCT |
| | | | PRRYAYWYQQRSGQAPVLVIYEDNKR | (Human) | (Human) | (Human) | (Human) | | ASNCYGNPM |
| | | | PSGIPERFSAFSSGTRATLTISGAQVED | | | | | | DV |
| | | | QADYYCYSTDSTANYKVFGGGTKLTVL | | | | | | |
| | mBG17 | 2038 | DIVMSQSPSSLAVSVGEKITMSCKSSQ | IGHV6-6 | IGHJ4 | IGKV8-30 | IGKJ1 | 3077 | TRSAMDY |
| | | | SLLYTSDQKNYLAWFQQKPGQSPKLLI | (Mouse) | (Mouse) | (Mouse) | (Mouse) | | |
| | | | FWASTRDSGVPDRFTGSGSGTDFTLTI | | | | | | |
| | | | SSVKAEDLAVYYCQQFYNYPRTFGGG | | | | | | |
| | | | TKLEIK | | | | | | |
| | mBG21 | 2039 | IVMTQTPKFLLVSAGDRVTITCKASQS | IGHV9-2- | IGHJ3 | IGKV6-32 | IGKJ1 | 3078 | ALRR |
| | | | VSNDVAWFQQKPGQSPKKLLIYFASN | 1 | (Mouse) | (Mouse) | (Mouse) | | |
| | | | RYTGVPDRFTGSGYGTDFTFTITTVQA | (Mouse) | | | | | |
| | | | EDLAVYFCQQDYSSPWTFGGGTKLEIK | | | | | | |
| | mBG22 | 2040 | IVMTQTPKFLLVSAGDRVTITCKASQS | IGHV9-2- | IGHJ3 | IGKV6-32 | IGKJ1 | 3079 | ALRR |
| | | | VSNDVAWFQQKPGQSPKLLIYFASNR | 1 | (Mouse) | (Mouse) | (Mouse) | | |
| | | | YTGVPDRFTGSGYGTDFTFTITTVQAE | (Mouse) | | | | | |
| | | | DLAVYFCQQDYSSPWTFGGGTKLEIK | | | | | | |
| | mBG57 | 2041 | DIVMTQAAPSEPVTPGESVSISCGSSK | IGHV9-2- | IGHJ3 | IGKV2-137 | IGKJ5 | 3080 | ALRR |
| | | | SLLHSNDNTYLYWFLQRPGQSPQLLIY | 1 | (Mouse) | (Mouse) | (Mouse) | | |
| | | | RMSNLASGVPDRFTGSGSGTAFTLRIS | (Mouse) | | | | | |
| | | | RVEAEDVGVYYCMQHLENPLGVRWR | | | | | | |
| | | | HQAGNQT | | | | | | |
| | mBG67 | 2042 | QIVLTQSPAIMSASLGERVTMTCTASS | IGHV5-6- | IGHJ1 | IGKV4-74 | IGKJ1 | 3081 | ARIYDFDEDY |
| | | | SVSSSYLHWYQQKPGSSPKLWIYSTSN | 3 | (Mouse) | (Mouse) | (Mouse) | | FDV |
| | | | LASGVPARFSGSGSGTSYSLTISSMEAE | (Mouse) | | | | | |
| | | | DAATYYCLQYHRSPWTFGGGTKLEIK | | | | | | |
| | MD17 | | ND | IGHV3-64 | ND | IGKV1-39 | ND | 3082 | VKDQDSSSW |
| | | | | (Human) | | (Human) | | | YDAFDI |
| | MD29 | | ND | IGHV3-64 | ND | IGKV1-39 | ND | 3083 | VKDQDSSSW |
| | | | | (Human) | | (Human) | | | YDAFDI |
| | MD45 | | ND | IGHV3-53 | ND | IGKV3-20 | ND | 3084 | ARDLSVRGG |
| | | | | (Human) | | (Human) | | | MDV |
| | MD47 | | ND | IGHV3-23 | ND | IGLV3-21 | ND | 3085 | AKDLVTAPSY |
| | | | | (Human) | | (Human) | | | EAFDI |
| | MD62 | | ND | IGHV3-53 | ND | IGKV1-12 | ND | 3086 | ARDLQYYGM |
| | | | | (Human) | | (Human) | | | DV |
| | MD63 | | ND | IGHV3-64 | ND | IGKV1-39 | ND | 3087 | VKDQDSNSW |
| | | | | (Human) | | (Human) | | | YDAFDI |
| | MD65 | | ND | IGHV3-66 | ND | IGKV3-20 | ND | 3088 | ARDLAVAGAF |
| | | | | (Human) | | (Human) | | | DI |
| | MD67 | | ND | IGHV3-53 | ND | IGKV3-20 | ND | 3089 | ARDLSVRGG |
| | | | | (Human) | | (Human) | | | MDV |
| | mNb6 | | ND | ND | ND | ND | ND | | ND |
| | mNb6-tri | | ND | ND | ND | ND | ND | | ND |
| | MnCit3p1_ | 2043 | QAVVTQEPSLTVSPGGTVTLTCGSSTG | IGHV3-23 | IGHJ4 | IGLV7-46 | IGU3 | 3090 | ALASGSYFGG |
| | G9 | | PVTSDHYPYWFQQKPGQAPTTLIYDT | (Human) | (Human) | (Human) | (Human) | | ANY |
| | | | NNKHSWTPARFSGSLLGGKAALTLSG | | | | | | |
| | | | AQPEDEAEYYCLLSYTGARVFGGGTKL | | | | | | |
| | | | TVL | | | | | | |
| | MnC2t1p1_ | 2044 | DIQMTQSPSSVSASVGDRVTITCRASQ | IGHV3-66 | IGHJ4 | IGKV1D-12 | IGKJ1 | 3091 | ATGARFGESP |
| | A3 | | GISSWLAWYQQKPGKAPKLLIYAASSL | (Human) | (Human) | (Human) | (Human) | | FDY |
| | | | QSGVPSRFSGSGSGTDFTLTISSLQPED | | | | | | |
| | | | FATYYCQQANSFPGTFGQGTKVEIK | | | | | | |
| | MnC2t1p1_ | 2045 | DIQMTQSPSSVSASVGDRVTITCRASQ | IGHV3-66 | IGHJ4 | IGKV1D-12 | IGKJ1 | 3092 | ATGARFGESP |
| | C5 | | GISSWLAWYQQKPGKAPKLLIYAASSL | (Human) | (Human) | (Human) | (Human) | | FDY |
| | | | QSGVPSRFSGSGSGTDFTLTISSLQPED | | | | | | |
| | | | FATYYCQQANSFPGTFGQGTKVEIK | | | | | | |
| | MnC2t2p1_ | 2046 | DIQMTQSPSSLSASVGDRVTITCRASQ | IGHV1-69 | IGHJ5 | IGKV1-39 | IGKJ2 | 3093 | AREGGLDYFG |
| | C11 | | NISSYLNWYQQKPGKAPNLLIYAASSL | (Human) | (Human) | (Human) | (Human) | | SRNSGWTYT |
| | | | QSGVPPRFSGSGSGTDFTLTISSLQPED | | | | | | WFDP |
| | | | FATYYCQQSYSTLYSFGQGTKLEIK | | | | | | |
| | MnC4t1p1_ | 2047 | DIQMTQSPSSLSASVGDRVTITCRASQ | IGHV4-39 | IGHJ3 | IGKV1-17 | IGKJ3 | 3094 | ARGVNYYDR |
| | A10 | | GIRNDLGWYEQKPGKAPKRLIYAASSL | (Human) | (Human) | (Human) | (Human) | | NGYYRNDGF |
| | | | ESGVPSRFSGSGSGTEFTLTISSLQPED | | | | | | DI |
| | | | FATYYCLQHNTYPFTFGPGTRVDIK | | | | | | |
| | MnC4t1p1_ | 2048 | EIVLTQSPGTLSLSPGERATLSCRASQS | IGHV3-48 | IGHJ4 | IGKV3-20 | IGKJ1 | 3095 | ASSKGFCSGG |
| | A11 | | VSSSYLAWYQQKPGQAPRLLIYGVSSR | (Human) | (Human) | (Human) | (Human) | | SCSDY |
| | | | ATGIPDRFSGSGSGTDFTLTISRLEPED | | | | | | |
| | | | FVVYYCHQYGSSPWTFGQGTKVEIK | | | | | | |
| | MnC4t2p1_ | 2049 | DIQMIQSPSSVSASVGDRVTITCRASQ | IGHV3-9 | IGHJ2 | IGKV1-12 | IGKJ3 | 3096 | AKDLRRQDYY |
| | B3 | | GISSWLAWYQQKPGKAPKLLIYAASTL | (Human) | (Human) | (Human) | (Human) | | ADWYFDL |
| | | | LSAVPSRFSGSGSGTDFTLTISSLQPED | | | | | | |
| | | | FATYYCQQGNSFPFTFGPGTIVDV | | | | | | |
| | MnC4t2p1_ | 2050 | DIQMTQSPSSLSASVGDRVTITCRASQ | IGHV4-39 | IGHJ3 | IGKV1-17 | IGKJ3 | 3097 | ARGVNYYDR |
| | D10 | | GIRNDLGWYEQKPGKAPKRLIYAASSL | (Human) | (Human) | (Human) | (Human) | | NGYYRNDGF |
| | | | ESGVPSRFSGSGSGTEFTLTISSLQPED | | | | | | DI |
| | | | FATYYCLQHNTYPFTFGPGTRVDIK | | | | | | |
| | MnC4t2p1_ | 2051 | DIQMTQSPSSVSASVGDRVTITCRASQ | IGHV3-9 | IGHJ2 | IGKV1-12 | IGKJ3 | 3098 | AKDLRRQDYY |
| | E6 | | GISSWLAWYQQKPGKAPKLLIYAASTL | (Human) | (Human) | (Human) | (Human) | | ADWYFDL |
| | | | LSAVPSRFSGSGSGTDFTLTISSLQPED | | | | | | |
| | | | FATYYCQQGNSFPFTFGPGTKVDV | | | | | | |
| | MnC4t2p1_ | 2052 | DIQMTQSPSSLSASVGDRVTITCRASQ | IGHV4-39 | IGHJ3 | IGKV1-17 | IGKJ3 | 3099 | ARGVNYYDR |
| | F5 | | GIRNDLGWYEQKPGKAPKRLIYAASSL | (Human) | (Human) | (Human) | (Human) | | NGYYRNDGF |
| | | | ESGVPSRFSGSGSGTEFTLTISSLQPED | | | | | | DI |
| | | | FATYYCLQHNTYPFTFGPGTRVDIK | | | | | | |
| | MnC4t2p2_ | 2053 | EIVLTQSPGTLSLSPGERATLSCRASHS | IGHV7-4- | IGHJ4 | IGKV3-20 | IGKJ5 | 3100 | AKIGSRNSLG |
| | A4 | | VDRSYLAWYQQKPGLAPRLLIYGASSR | 1 | (Human) | (Human) | (Human) | | V |
| | | | ATGIPDRFSGSGSGTDFTLTISRLEPED | (Human) | | | | | |
| | | | FALYYCQHFGTSSVTFGRGTRLEIK | | | | | | |
| | MnC5t2p1_ | 2054 | EIVLTQSPGTLSLSPGERATLSCRASQS | IGHV1-58 | IGHJ3 | IGKV3-20 | IGKJ1 | 3101 | AAPRCSGGSC |
| | G1 | | VSSSYLAWYQHKPGQAPRLLICGASSR | (Human) | (Human) | (Human) | (Human) | | YDGFDI |
| | | | ATGIPDRFSGSGSGTGFTLTISRLEPED | | | | | | |
| | | | FAVYYCQQYGSSPWTFGQGTKVEIK | | | | | | |
| | MR10 | | N/A | IGHV3S53 | IGHJ4 | N/A | N/A | 3102 | NVKDEGATTK |
| | | | | (Alpaca) | (Alpaca) | | | | VYDY |
| | MR14 | | N/A | IGHV3S53 | IGHJ4 | N/A | N/A | 3103 | NVKDWGAA |
| | | | | (Alpaca) | (Alpaca) | | | | NKYYDY |
| | MR17 | | N/A | IGHV3S53 | IGHJ4 | N/A | N/A | 3104 | NVKDDGQLA |
| | | | | (Alpaca) | (Alpaca) | | | | YHYDY |
| | MR17_K56W | | N/A | IGHV3S53 | IGHJ4 | N/A | N/A | 3105 | NVKDDGQLA |
| | | | | (Alpaca) | (Alpaca) | | | | YHYDY |
| | MR17_K99W | | N/A | IGHV3S5 | IGHJ4 | N/A | N/A | 3106 | NVWDDGQL |
| | | | | (Alpaca) | (Alpaca) | | | | AYHYDY |
| | MR17_K99Y | | N/A | IGHV3S53 | IGHJ4 | N/A | N/A | 3107 | NVYDDGQLA |
| | | | | (Alpaca) | (Alpaca) | | | | YHYDY |
| | MR2 | | N/A | IGHV3S53 | IGHJ4 | N/A | N/A | 3108 | NVKDYGWYN |
| | | | | (Alpaca) | (Alpaca) | | | | SQYDY |
| | MR3 | | N/A | IGHV3S53 | IGHJ4 | N/A | N/A | 3109 | NVKDYGAAS |
| | | | | (Alpaca) | (Alpaca) | | | | WEYDY |
| | MR4 | | N/A | IGHV3S53 | IGHJ4 | N/A | N/A | 3110 | NVKDFGGHQ |
| | | | | (Alpaca) | (Alpaca) | | | | AYYDY |
| | MR6 | | N/A | IGHV3S53 | IGHJ4 | N/A | N/A | 3111 | NVKDEGDTS |
| | | | | (Alpaca) | (Alpaca) | | | | ASYDY |
| | MR7 | | N/A | IGHV3S53 | IGHJ4 | N/A | N/A | 3112 | NVKDEGYFSD |
| | | | | (Alpaca) | (Alpaca) | | | | EYDY |
| | MR8 | | N/A | IGHV3S53 | IGHJ4 | N/A | N/A | 3113 | NVKDWGSSN |
| | | | | (Alpaca) | (Alpaca) | | | | QYYDY |
| | Nb11 | | ND | ND | ND | ND | ND | | ND |
| | Nb11-59 | | ND | ND | ND | ND | ND | | ND |
| | Nb3 | | ND | ND | ND | ND | ND | | ND |
| | Nb3-bi | | ND | ND | ND | ND | ND | | ND |
| | Nb3-tri | | ND | ND | ND | ND | ND | | ND |
| | Nb4-43 | | ND | ND | ND | ND | ND | | ND |
| | Nb6 | | ND | ND | ND | ND | ND | | ND |
| | Nb6-tri | | ND | ND | ND | ND | ND | | ND |
| | NIH- | | N/A | IGHV3-3 | IGHJ7 | N/A | N/A | 3114 | AAVPSTYYNG |
| | CoVnb- | | | (Alpaca) | (Alpaca) | | | | SYYYTCHPGG |
| | 101 | | | | | | | | MDY |
| | NIH- | | N/A | IGHV3-3 | IGHJ7 | N/A | N/A | 3115 | AAVPSTYYSG |
| | CoVnb- | | | (Alpaca) | (Alpaca) | | | | TYYYNCHPGG |
| | 102 | | | | | | | | MDY |
| | NIH- | | N/A | IGHV3-3 | IGHJ7 | N/A | N/A | 3116 | ATAPGTYYKG |
| | CoVnb- | | | (Alpaca) | (Alpaca) | | | | SYYPMCHYYG |
| | 103 | | | | | | | | MDY |
| | NIH- | | N/A | IGHV3-3 | IGHJ7 | N/A | N/A | 3117 | ATRPLTYYSGS |
| | CoVnb- | | | (Alpaca) | (Alpaca) | | | | YYTTCSDYGM |
| | 104 | | | | | | | | DY |
| | NIH- | | N/A | IGHV3-3 | IGHJ7 | N/A | N/A | 3118 | AAVPSTYYSG |
| | CoVnb- | | | (Alpaca) | (Alpaca) | | | | SYYYTCHPGG |
| | 105 | | | | | | | | MDY |
| | NIH- | | N/A | IGHV3-3 | IGHJ7 | N/A | N/A | 3119 | AAVPSTYYSG |
| | CoVnb- | | | (Alpaca) | (Alpaca) | | | | SYYYTCHPGG |
| | 106 | | | | | | | | MDY |
| | NIH- | | N/A | IGHV3-3 | IGHJ7 | N/A | N/A | 3120 | AAVPSTYYSG |
| | CoVnb- | | | (Alpaca) | (Alpaca) | | | | SYYYTCHPGG |
| | 107 | | | | | | | | MDY |
| | NIH- | | N/A | IGHV3-3 | IGHJ7 | N/A | N/A | 3121 | AAVPSTYYSG |
| | CoVnb- | | | (Alpaca) | (Alpaca) | | | | SYYYTCHPGG |
| | 108 | | | | | | | | MDY |
| | NIH- | | N/A | IGHV3-3 | IGHJ7 | N/A | N/A | 3122 | ASFPSTYYSGS |
| | CoVnb- | | | (Alpaca) | (Alpaca) | | | | YYYTCHPGG |
| | 109 | | | | | | | | MDY |
| | NIH- | | N/A | IGHV3-3 | IGHJ7 | N/A | N/A | 3123 | AAVPSTYYSG |
| | CoVnb- | | | (Alpaca) | (Alpaca) | | | | TYYYNCHPGA |
| | 110 | | | | | | | | MHY |
| | NIH- | | N/A | IGHV3-3 | IGHJ7 | N/A | N/A | 3124 | AAVPSTYYSG |
| | CoVnb- | | | (Alpaca) | (Alpaca) | | | | TYYYNCHPGA |
| | 111 | | | | | | | | MHY |
| | NIH- | | N/A | IGHV3-3 | IGHJ7 | N/A | N/A | 3125 | AAVPSTYYSG |
| | CoVnb- | | | (Alpaca) | (Alpaca) | | | | TYYYTCHPGG |
| | 112 | | | | | | | | MDY |
| | NIH- | | N/A | IGHV3-3 | IGHJ7 | N/A | N/A | 3126 | AAVPSTYYSG |
| | CoVnb- | | | (Alpaca) | (Alpaca) | | | | TYYYTCHPGG |
| | 113 | | | | | | | | MDY |
| | P2B-2F6 | 2055 | QSALTQPPSASGSPGQSVTISCTGTSS | IGHV4- | IGHJ3 | IGLV2-8 | IGU3 | 3127 | ARAVVGIVVV |
| | | | DVGGYNYVSWYQQHPGKAPKLMIYE | 382 | (Human) | (Human) | (Human) | | PAAGRRAFDI |
| | | | VSKRPSGVPDRFSGSKSGNTASLTVSG | (Human) | | | | | |
| | | | LQAEDEADYYCSSYAGSNNLVFGGGT | | | | | | |
| | | | KLTVL | | | | | | |
| | S110 | | ND | IGHV3-30 | ND | IGKV2-30 | ND | 3128 | AKDRFQFARS |
| | | | | (Human) | | (Human) | | | WYGDYFDY |
| | S124 | | ND | IGHV2-26 | ND | IGKV1-39 | ND | 3129 | ARINTAAYDY |
| | | | | (Human) | | (Human) | | | DSTTFDI |
| | S303 | | ND | IGHV3-23 | ND | IGKV1-5 | ND | 3130 | ARERDDIFPM |
| | | | | (Human) | | (Human) | | | GLNAFDI |
| | S304 | | ND | IGHV3-13 | ND | IGKV1-39 | ND | 3131 | ARGDSSGYYY |
| | | | | (Human) | | (Human) | | | YFDY |
| | S306 | | ND | IGHV1-18 | ND | IGKV3-11 | ND | 3132 | ASDYFDSSGY |
| | | | | (Human) | | (Human) | | | YHSFDY |
| | S309 | 2056 | EIVLTQSPGTLSLSPGERATLSCRASQT | IGHV1-18 | IGHJ4 | IGKV3-20 | IGKJ4 | 3133 | ARDYTRGAW |
| | | | VSSTSLAWYQQKPGQAPRLLIYGASSR | (Human) | (Human) | (Human) | (Human) | | FGESLIGGFD |
| | | | ATGIPDRFSGSGSGTDFTLTISRLEPED | | | | | | N |
| | | | FAVYYCQQHDTSLTFGGGTKVEIK | | | | | | |
| | S310 | | ND | IGHV1-69 | ND | IGLV2-23 | ND | 3134 | ATRTYDSSGY |
| | | | | (Human) | | (Human) | | | RPYYYGLDV |
| | S315 | | ND | IGHV3-7 | ND | IGLV3-25 | ND | 3135 | ARDLWWND |
| | | | | (Human) | | (Human) | | | QAHYYGMDV |
| | Sb#1 | | N/A | IGHV3-3 | IGHJ4 | N/A | N/A | 3136 | RVFVGWHY |
| | | | | (Alpaca) | (Alpaca) | | | | |
| | Sb#10 | | N/A | IGHV3-3 | IGHJ4 | N/A | N/A | 3137 | TVYVGYEY |
| | | | | (Alpaca) | (Alpaca) | | | | |
| | Sb#11 | | N/A | IGHV3-3 | IGHJ4 | N/A | N/A | 3138 | EVEVGKWY |
| | | | | (Alpaca) | (Alpaca) | | | | |
| | Sb#12 | | N/A | IGHV3-3 | IGHJ5 | N/A | N/A | 3139 | YVWVGQEY |
| | | | | (Alpaca) | (Alpaca) | | | | |
| | Sb#13 | | N/A | IGHV3-3 | IGHJ4 | N/A | N/A | 3140 | WVIVGEYY |
| | | | | (Alpaca) | (Alpaca) | | | | |
| | Sb#14 | | N/A | IGHV3-3 | IGHJ4 | N/A | N/A | 3141 | YVYVGSSY |
| | | | | (Alpaca) | (Alpaca) | | | | |
| | Sb#15 | | N/A | IGHV3-3 | IGHJ4 | N/A | N/A | 3142 | FVYVGRSY |
| | | | | (Alpaca) | (Alpaca) | | | | |
| | Sb#16 | | N/A | IGHV3S53 | IGHJ4 | N/A | N/A | 3143 | IVWVGAQY |
| | | | | (Alpaca) | (Alpaca) | | | | |
| | Sb#17 | | N/A | IGHV3-3 | IGHJ4 | N/A | N/A | 3144 | HVWVGSLY |
| | | | | (Alpaca) | (Alpaca) | | | | |
| | Sb#18 | | N/A | IGHV3-3 | IGHJ4 | N/A | N/A | 3145 | YVYVGASY |
| | | | | (Alpaca) | (Alpaca) | | | | |
| | Sb#19 | | N/A | IGHV3-3 | IGHJ4 | N/A | N/A | 3146 | YVYVGSSY |
| | | | | (Alpaca) | (Alpaca) | | | | |
| | Sb#2 | | N/A | IGHV3-3 | IGHJ4 | N/A | N/A | 3147 | RAVYVGMHY |
| | | | | (Alpaca) | (Alpaca) | | | | |
| | Sb#20 | | N/A | IGHV3-3 | IGHJ4 | N/A | N/A | 3148 | YVYVGKSY |
| | | | | (Alpaca) | (Alpaca) | | | | |
| | Sb#21 | | N/A | IGHV3-3 | IGHJ4 | N/A | N/A | 3149 | FVGVGTHY |
| | | | | (Alpaca) | (Alpaca) | | | | |
| | Sb#22 | | N/A | IGHV3-3 | IGHJ4 | N/A | N/A | 3150 | FVYVGKSY |
| | | | | (Alpaca) | (Alpaca) | | | | |
| | Sb#23 | | N/A | IGHV3-3 | IGHJ4 | N/A | N/A | 3151 | FVYVGKSY |
| | | | | (Alpaca) | (Alpaca) | | | | |
| | Sb#25 | | N/A | IGHV3S53 | IGHJ4 | N/A | N/A | 3152 | NVKDFGTHH |
| | | | | (Alpaca) | (Alpaca) | | | | YAYDY |
| | Sb#26 | | N/A | IGHV3S53 | IGHJ4 | N/A | N/A | 3153 | NVKDKGMAV |
| | | | | (Alpaca) | (Alpaca) | | | | QWYDY |
| | Sb#27 | | N/A | IGHV3S53 | IGHJ4 | N/A | N/A | 3154 | NVKDEGDMF |
| | | | | (Alpaca) | (Alpaca) | | | | TAYDY |
| | Sb#28 | | N/A | IGHV3-3 | IGHJ4 | N/A | N/A | 3155 | NVKDSGQWR |
| | | | | (Alpaca) | (Alpaca) | | | | QEYDY |
| | Sb#29 | | N/A | IGHV3S53 | IGHJ4 | N/A | N/A | 3156 | NVKDFGYTW |
| | | | | (Alpaca) | (Alpaca) | | | | HEYDY |
| | Sb#3 | | N/A | IGHV3-3 | IGHJ4 | N/A | N/A | 3157 | VVWVGHNY |
| | | | | (Alpaca) | (Alpaca) | | | | |
| | Sb#30 | | N/A | IGHV3-3 | IGHJ4 | N/A | N/A | 3158 | NVKDYGQAH |
| | | | | (Alpaca) | (Alpaca) | | | | AYYDY |
| | Sb#31 | | N/A | IGHV3-3 | IGHJ4 | N/A | N/A | 3159 | NVKDTGTTED |
| | | | | (Alpaca) | (Alpaca) | | | | YDY |
| | Sb#32 | | N/A | IGHV3-3 | IGHJ4 | N/A | N/A | 3160 | NVKDAGRVY |
| | | | | (Alpaca) | (Alpaca) | | | | NSYDY |
| | Sb#33 | | N/A | IGHV3S53 | IGHJ4 | N/A | N/A | 3161 | NVKDTGTYRF |
| | | | | (Alpaca) | (Alpaca) | | | | YYDY |
| | Sb#34 | | N/A | IGHV3S53 | IGHJ4 | N/A | N/A | 3162 | NVKDAGVYN |
| | | | | (Alpaca) | (Alpaca) | | | | RYYDY |
| | Sb#35 | | N/A | IGHV3-3 | IGHJ4 | N/A | N/A | 3163 | NVKDWGFAS |
| | | | | (Alpaca) | (Alpaca) | | | | HAYDY |
| | Sb#36 | | N/A | IGHV3S53 | IGHJ4 | N/A | N/A | 3164 | NVKDFGWQ |
| | | | | (Alpaca) | (Alpaca) | | | | HQEYDY |
| | Sb#37 | | N/A | IGHV3-3 | IGHJ4 | N/A | N/A | 3165 | NVKDSGSFN |
| | | | | (Alpaca) | (Alpaca) | | | | QAYDY |
| | Sb#38 | | N/A | IGHV3S53 | IGHJ4 | N/A | N/A | 3166 | NVKDYGVHF |
| | | | | (Alpaca) | (Alpaca) | | | | KRYDY |
| | Sb#39 | | N/A | IGHV3-3 | IGHJ4 | N/A | N/A | 3167 | NVKDAGNTT |
| | | | | (Alpaca) | (Alpaca) | | | | SAYDY |
| | Sb#4 | | N/A | IGHV3-3 | IGHJ4 | N/A | N/A | 3168 | EVQVGAWY |
| | | | | (Alpaca) | (Alpaca) | | | | |
| | Sb#40 | | N/A | IGHV3S53 | IGHJ4 | N/A | N/A | 3169 | NVKDIDAEAY |
| | | | | (Alpaca) | (Alpaca) | | | | AYDY |
| | Sb#41 | | N/A | IGHV3-3 | IGHJ4 | N/A | N/A | 3170 | NVKDSGQWR |
| | | | | (Alpaca) | (Alpaca) | | | | VQYDY |
| | Sb#42 | | N/A | IGHV3S53 | IGHJ4 | N/A | N/A | 3171 | NVKDHGAQN |
| | | | | (Alpaca) | (Alpaca) | | | | QMYDY |
| | Sb#45 | | N/A | IGHV3S53 | IGHJ4 | N/A | N/A | 3172 | NVKDVGHHY |
| | | | | (Alpaca) | (Alpaca) | | | | EYYDY |
| | Sb#46 | | N/A | IGHV3S53 | IGHJ4 | N/A | N/A | 3173 | NVKDKGQM |
| | | | | (Alpaca) | (Alpaca) | | | | RAAYDY |
| | Sb#47 | | N/A | IGHV3-3 | IGHJ4 | N/A | N/A | 3174 | NVKDYGSSYY |
| | | | | (Alpaca) | (Alpaca) | | | | KYDY |
| | Sb#48 | | N/A | IGHV3S53 | IGHJ4 | N/A | N/A | 3175 | NVKDAGSSY |
| | | | | (Alpaca) | (Alpaca) | | | | WDYD |
| | Sb#49 | | N/A | IGHV3-3 | IGHJ4 | N/A | N/A | 3176 | AAARWGRTK |
| | | | | (Alpaca) | (Alpaca) | | | | PLNTYYYSY |
| | Sb#5 | | N/A | IGHV3-3 | IGHJ4 | N/A | N/A | 3177 | RVHVGAHY |
| | | | | (Alpaca) | (Alpaca) | | | | |
| | Sb#50 | | N/A | IGHV3-3 | IGHJ4 | N/A | N/A | 3178 | AAATEGHAH |
| | | | | (Alpaca) | (Alpaca) | | | | ALYRLHYY |
| | Sb#51 | | N/A | IGHV3-3 | IGHJ4 | N/A | N/A | 3179 | RVWVGTHY |
| | | | | (Alpaca) | (Alpaca) | | | | |
| | Sb#52 | | N/A | IGHV3-3 | IGHJ4 | N/A | N/A | 3180 | AAAYVGAEN |
| | | | | (Alpaca) | (Alpaca) | | | | PLPYSMYGY |
| | Sb#53 | | N/A | IGHV3-3 | IGHJ4 | N/A | N/A | 3181 | AAADYGASD |
| | | | | (Alpaca) | (Alpaca) | | | | PLWFIHYLY |
| | Sb#55 | | N/A | IGHV3-3 | IGHJ4 | N/A | N/A | 3182 | AAANYGSNFP |
| | | | | (Alpaca) | (Alpaca) | | | | LAEEDYWY |
| | Sb#56 | | N/A | IGHV3-3 | IGHJ4 | N/A | N/A | 3183 | AAAYYFGDDI |
| | | | | (Alpaca) | (Alpaca) | | | | PLWWEAYSY |
| | Sb#58 | | N/A | IGHV3-3 | IGHJ4 | N/A | N/A | 3184 | AAARWGRH |
| | | | | (Alpaca) | (Alpaca) | | | | MPLSATEYSY |
| | Sb#59 | | N/A | IGHV3-3 | IGHJ4 | N/A | N/A | 3185 | AAAAWGNSA |
| | | | | (Alpaca) | (Alpaca) | | | | PLTTYRYYY |
| | Sb#6 | | N/A | IGHV3-3 | IGHJ4 | N/A | N/A | 3186 | YVYVGAQY |
| | | | | (Alpaca) | (Alpaca) | | | | |
| | Sb#61 | | N/A | IGHV3-3 | IGHJ4 | N/A | N/A | 3187 | AAADWGYD |
| | | | | (Alpaca) | (Alpaca) | | | | WPLWDEWY |
| | | | | | | | | | WY |
| | Sb#62 | | N/A | IGHV3-3 | IGHJ4 | N/A | N/A | 3188 | AAANYGANY |
| | | | | (Alpaca) | (Alpaca) | | | | PLYSQQYSY |
| | Sb#63 | | N/A | IGHV3-3 | IGHJ4 | N/A | N/A | 3189 | AAANYGANE |
| | | | | (Alpaca) | (Alpaca) | | | | PLQYTHYNY |
| | Sb#64 | | N/A | IGHV3-3 | IGHJ4 | N/A | N/A | 3190 | AAASYGAAHP |
| | | | | (Alpaca) | (Alpaca) | | | | LSIMRYYY |
| | Sb#65 | | N/A | IGHV3-3 | IGHJ4 | N/A | N/A | 3191 | AAASYGANFP |
| | | | | (Alpaca) | (Alpaca) | | | | LKASDYSY |
| | Sb#66 | | N/A | IGHV3-3 | IGHJ4 | N/A | N/A | 3192 | AAADWGYD |
| | | | | (Alpaca) | (Alpaca) | | | | WPLWDEWY |
| | | | | | | | | | WY |
| | Sb#67 | | N/A | IGHV3-3 | IGHJ4 | N/A | N/A | 3193 | AAATWGHS |
| | | | | (Alpaca) | (Alpaca) | | | | WPLYNDEYW |
| | | | | | | | | | Y |
| | Sb#68 | | N/A | IGHV3-3 | IGHJ4 | N/A | N/A | 3194 | AAAAWGYA |
| | | | | (Alpaca) | (Alpaca) | | | | WPLHQDDY |
| | | | | | | | | | WY |
| | Sb#69 | | N/A | IGHV3-3 | IGHJ5 | N/A | N/A | 3195 | AAATWGYSW |
| | | | | (Alpaca) | (Alpaca) | | | | PLIAEYNWY |
| | Sb#7 | | N/A | IGHV3-3 | IGHJ4 | N/A | N/A | 3196 | FVKVGNWY |
| | | | | (Alpaca) | (Alpaca) | | | | |
| | Sb#71 | | N/A | IGHV3-3 | IGHJ4 | N/A | N/A | 3197 | AAANWGYS |
| | | | | (Alpaca) | (Alpaca) | | | | WPLYEADDW |
| | | | | | | | | | Y |
| | Sb#8 | | N/A | IGHV3-3 | IGHJ4 | N/A | N/A | 3198 | YVYVGGSY |
| | | | | (Alpaca) | (Alpaca) | | | | |
| | Sb#9 | | N/A | IGHV3-3 | IGHJ4 | N/A | N/A | 3199 | RVFVGMHY |
| | | | | (Alpaca) | (Alpaca) | | | | |
| | Sb100 | | N/A | IGHV3-3 | IGHJ4 | N/A | N/A | 3200 | AAANWGYS |
| | | | | (Alpaca) | (Alpaca) | | | | WPLYQTEYW |
| | | | | | | | | | Y |
| | Sb12 | | N/A | IGHV3-3 | IGHJ4 | N/A | N/A | 3201 | YVKVGEWY |
| | | | | (Alpaca) | (Alpaca) | | | | |
| | Sb13 | | N/A | IGHV3-3 | IGHJ4 | N/A | N/A | 3202 | YVVVGWGY |
| | | | | (Alpaca) | (Alpaca) | | | | |
| | Sb15 | | N/A | IGHV3-3 | IGHJ4 | N/A | N/A | 3203 | FVVVGNGY |
| | | | | (Alpaca) | (Alpaca) | | | | |
| | Sb16 | | N/A | IGHV3-3 | IGHJ4 | N/A | N/A | 3204 | YVYVGGSY |
| | | | | (Alpaca) | (Alpaca) | | | | |
| | Sb17 | | N/A | IGHV3-3 | IGHJ4 | N/A | N/A | 3205 | LVYVGATY |
| | | | | (Alpaca) | (Alpaca) | | | | |
| | Sb2 | | N/A | IGHV3-3 | IGHJ4 | N/A | N/A | 3206 | YVYVGASY |
| | | | | (Alpaca) | (Alpaca) | | | | |
| | Sb21 | | N/A | IGHV3-3 | IGHJ4 | N/A | N/A | 3207 | YVYVGGSY |
| | | | | (Alpaca) | (Alpaca) | | | | |
| | Sb22 | | N/A | IGHV3-3 | IGHJ4 | N/A | N/A | 3208 | YVYVGASY |
| | | | | (Alpaca) | (Alpaca) | | | | |
| | Sb23 | | N/A | IGHV3-3 | IGHJ4 | N/A | N/A | 3209 | AVQVGYWY |
| | | | | (Alpaca) | (Alpaca) | | | | |
| | Sb25 | | N/A | IGHV3-3 | IGHJ4 | N/A | N/A | 3210 | YVYVGAGY |
| | | | | (Alpaca) | (Alpaca) | | | | |
| | Sb27 | | N/A | IGHV3-3 | IGHJ4 | N/A | N/A | 3211 | YVWVGRSY |
| | | | | (Alpaca) | (Alpaca) | | | | |
| | Sb28 | | N/A | IGHV3-3 | IGHJ4 | N/A | N/A | 3212 | YVYVGSSY |
| | | | | (Alpaca) | (Alpaca) | | | | |
| | Sb30 | | N/A | IGHV3-3 | IGHJ4 | N/A | N/A | 3213 | YVYVGESY |
| | | | | (Alpaca) | (Alpaca) | | | | |
| | Sb32 | | N/A | IGHV3-3 | IGHJ4 | N/A | N/A | 3214 | VVWVGEVY |
| | | | | (Alpaca) | (Alpaca) | | | | |
| | Sb37 | | N/A | IGHV3S53 | IGHJ4 | N/A | N/A | 3215 | NVKDEGNTT |
| | | | | (Alpaca) | (Alpaca) | | | | AYYDY |
| | Sb38 | | N/A | IGHV3S53 | IGHJ4 | N/A | N/A | 3216 | NVKDFGTQE |
| | | | | (Alpaca) | (Alpaca) | | | | HYYDY |
| | Sb39 | | N/A | IGHV3S53 | IGHJ4 | N/A | N/A | 3217 | NVKDFGGYR |
| | | | | (Alpaca) | (Alpaca) | | | | YYYDY |
| | Sb40 | | N/A | IGHV3S53 | IGHJ4 | N/A | N/A | 3218 | NVKDEGAIAK |
| | | | | (Alpaca) | (Alpaca) | | | | NYDY |
| | Sb42 | | N/A | IGHV3S53 | IGHJ4 | N/A | N/A | 3219 | NVKDEGYTGY |
| | | | | (Alpaca) | (Alpaca) | | | | YYDY |
| | Sb43 | | N/A | IGHV3-3 | IGHJ4 | N/A | N/A | 3220 | NVKDWGSQ |
| | | | | (Alpaca) | (Alpaca) | | | | DRYYDY |
| | Sb45 | | N/A | IGHV3S53 | IGHJ4 | N/A | N/A | 3221 | NVKDEGKSS |
| | | | | (Alpaca) | (Alpaca) | | | | QVYDY |
| | Sb46 | | N/A | IGHV3S53 | IGHJ4 | N/A | N/A | 3222 | NVKDVGNDQ |
| | | | | (Alpaca) | (Alpaca) | | | | KSYDY |
| | Sb47 | | N/A | IGHV3S53 | IGHJ4 | N/A | N/A | 3223 | NVKDWGTYS |
| | | | | (Alpaca) | (Alpaca) | | | | TYYDY |
| | Sb5 | | N/A | IGHV3-3 | IGHJ4 | N/A | N/A | 3224 | YVYVGASY |
| | | | | (Alpaca) | (Alpaca) | | | | |
| | Sb50 | | N/A | IGHV3S53 | IGHJ4 | N/A | N/A | 3225 | NVKDWGWL |
| | | | | (Alpaca) | (Alpaca) | | | | AQYYDY |
| | Sb52 | | N/A | IGHV3S53 | IGHJ4 | N/A | N/A | 3226 | NVKDEGMW |
| | | | | (Alpaca) | (Alpaca) | | | | QHYYDY |
| | Sb54 | | N/A | IGHV3S53 | IGHJ4 | N/A | N/A | 3227 | NVKDEGNSQ |
| | | | | (Alpaca) | (Alpaca) | | | | SHYDY |
| | Sb56 | | N/A | IGHV3S53 | IGHJ4 | N/A | N/A | 3228 | NVKDAGNSK |
| | | | | (Alpaca) | (Alpaca) | | | | ALYDY |
| | Sb57 | | N/A | IGHV3S53 | IGHJ4 | N/A | N/A | 3229 | NVKDWGRA |
| | | | | (Alpaca) | (Alpaca) | | | | GARYDY |
| | Sb58 | | N/A | IGHV3S53 | IGHJ4 | N/A | N/A | 3230 | NVKDMDRW |
| | | | | (Alpaca) | (Alpaca) | | | | RTTYDY |
| | Sb6 | | N/A | IGHV3-3 | IGHJ4 | N/A | N/A | 3231 | YVWVGNQY |
| | | | | (Alpaca) | (Alpaca) | | | | |
| | Sb60 | | N/A | IGHV3S53 | IGHJ4 | N/A | N/A | 3232 | NVKDWGYEY |
| | | | | (Alpaca) | (Alpaca) | | | | EGYDY |
| | Sb61 | | N/A | IGHV3S53 | IGHJ4 | N/A | N/A | 3233 | NVKDTGTYQ |
| | | | | (Alpaca) | (Alpaca) | | | | AWYDY |
| | Sb62 | | N/A | IGHV3S53 | IGHJ4 | N/A | N/A | 3234 | NVKDWGGY |
| | | | | (Alpaca) | (Alpaca) | | | | QWYYDY |
| | Sb63 | | N/A | IGHV3S53 | IGHJ4 | N/A | N/A | 3235 | NVKDYGAQA |
| | | | | (Alpaca) | (Alpaca) | | | | HYYDY |
| | Sb67 | | N/A | IGHV3S53 | IGHJ4 | N/A | N/A | 3236 | NVKDWGTYN |
| | | | | (Alpaca) | (Alpaca) | | | | SYYDY |
| | Sb7 | | N/A | IGHV3-3 | IGHJ4 | N/A | N/A | 3237 | LVYVGSTY |
| | | | | (Alpaca) | (Alpaca) | | | | |
| | Sb71 | | N/A | IGHV3-3 | IGHJ4 | N/A | N/A | 3238 | AAAHYGDNF |
| | | | | (Alpaca) | (Alpaca) | | | | PLAYQAYLY |
| | Sb75 | | N/A | IGHV3-3 | IGHJ4 | N/A | N/A | 3239 | AAARWGRDE |
| | | | | (Alpaca) | (Alpaca) | | | | PLYHYYYSY |
| | Sb76 | | N/A | IGHV3-3 | IGHJ4 | N/A | N/A | 3240 | NVKDIGAQEV |
| | | | | (Alpaca) | (Alpaca) | | | | HYDY |
| | Sb78 | | N/A | IGHV3-3 | IGHJ4 | N/A | N/A | 3241 | AAANYGNN |
| | | | | (Alpaca) | (Alpaca) | | | | WPLTGVNYW |
| | Sb8 | | N/A | IGHV3-3 | IGHJ4 | N/A | N/A | 3242 | YVWVGDSY |
| | | | | (Alpaca) | (Alpaca) | | | | |
| | Sb83 | | N/A | IGHV3-3 | IGHJ4 | N/A | N/A | 3243 | AAAKYGQNF |
| | | | | (Alpaca) | (Alpaca) | | | | PLSYHAYRY |
| | Sb84 | | N/A | IGHV3-3 | IGHJ4 | N/A | N/A | 3244 | AAARYGRSDP |
| | | | | (Alpaca) | (Alpaca) | | | | LHYHEYSY |
| | Sb85 | | N/A | IGHV3-3 | IGHJ4 | N/A | N/A | 3245 | AAASWGYTW |
| | | | | (Alpaca) | (Alpaca) | | | | PLYTYDYWY |
| | Sb88 | | N/A | IGHV3S53 | IGHJ4 | N/A | N/A | 3246 | NVKDSGQYR |
| | | | | (Alpaca) | (Alpaca) | | | | ENYDY |
| | Sb9 | | N/A | IGHV3-3 | IGHJ4 | N/A | N/A | 3247 | WVYVGDYY |
| | | | | (Alpaca) | (Alpaca) | | | | |
| | Sb90 | | N/A | IGHV3-3 | IGHJ4 | N/A | N/A | 3248 | AAARWGRQY |
| | | | | (Alpaca) | (Alpaca) | | | | PLTFVYYSY |
| | Sb93 | | N/A | IGHV3-3 | IGHJ4 | N/A | N/A | 3249 | AAARWGRTY |
| | | | | (Alpaca) | (Alpaca) | | | | PLSYMAYTY |
| | Sb94 | | N/A | IGHV3-3 | IGHJ4 | N/A | N/A | 3250 | AAARWGRYE |
| | | | | (Alpaca) | (Alpaca) | | | | PLHYAYYSY |
| | Sb95 | | N/A | IGHV3-3 | IGHJ4 | N/A | N/A | 3251 | AAASYGANW |
| | | | | (Alpaca) | (Alpaca) | | | | PLVSAAYTY |
| | Sb97 | | N/A | IGHV3-3 | IGHJ4 | N/A | N/A | 3252 | AAARYGHAQ |
| | | | | (Alpaca) | (Alpaca) | | | | APLHYFWYGY |
| | SR1 | | N/A | IGHV3-3 | IGHJ4 | N/A | N/A | 3253 | YVYVGFSY |
| | | | | (Alpaca) | (Alpaca) | | | | |
| | SR13 | | N/A | IGHV3-3 | IGHJ4 | N/A | N/A | 3254 | YVYVGSSY |
| | | | | (Alpaca) | (Alpaca) | | | | |
| | SR18 | | N/A | IGHV3-3 | IGHJ4 | N/A | N/A | 3255 | YVYVGASY |
| | | | | (Alpaca) | (Alpaca) | | | | |
| | SR31 | | N/A | IGHV3-3 | IGHJ4 | N/A | N/A | 3256 | AVMVGFWY |
| | | | | (Alpaca) | (Alpaca) | | | | |
| | SR34 | | N/A | IGHV3-3 | IGHJ4 | N/A | N/A | 3257 | YVYVGSSY |
| | | | | (Alpaca) | (Alpaca) | | | | |
| | SR38 | | N/A | IGHV3-3 | IGHJ4 | N/A | N/A | 3258 | AVHVGQTY |
| | | | | (Alpaca) | (Alpaca) | | | | |
| | SR4 | | N/A | IGHV3-3 | IGHJ4 | N/A | N/A | 3259 | YVWVGHTY |
| | | | | (Alpaca) | (Alpaca) | | | | |
| | SR5 | | N/A | IGHV3-3 | IGHJ4 | N/A | N/A | 3260 | YVYVGTSY |
| | | | | (Alpaca) | (Alpaca) | | | | |
| | SR7 | | N/A | IGHV3S53 | IGHJ4 | N/A | N/A | 3261 | YVYVGYSY |
| | | | | (Alpaca) | (Alpaca) | | | | |
| | Ty1 | | N/A | IGHV3S1 | IGHJ4 | N/A | N/A | 3262 | AIGLNLSSSSV |
| | | | | (Alpaca) | (Alpaca) | | | | |
| | VH-Fc- | | ND | ND | ND | ND | ND | | ND |
| | B01 | | | | | | | | |
| | VH2-A01- | | ND | ND | ND | ND | ND | | ND |
| | B01 | | | | | | | | |
| | VH2-A01- | | ND | ND | ND | ND | ND | | ND |
| | B02 | | | | | | | | |
| | VH3-B01 | | ND | ND | ND | ND | ND | | ND |
| | VHH-72 | | N/A | IGHV3-3 | IGHJ4 | N/A | N/A | 3263 | AAAGLGTVVS |
| | | | | (Alpaca) | (Alpaca) | | | | EWDYDYDY |
| | W23UACh | | N/A | IGHV3S53 | IGHJ4 | N/A | N/A | 3264 | HALIKNELGFL |
| | | | | (Alpaca) | (Alpaca) | | | | DY |
| | W25UACh | | N/A | IGHV3S53 | IGHJ4 | N/A | N/A | 3265 | HALIKNELGFL |
| | | | | (Alpaca) | (Alpaca) | | | | DY |
| | | | | | | | SEQ | | |
| | | | | | | | ID | | |
| | | | | | | Name | NO.: | CDRL3 | Sources |
| | | | | | | 0304-2F8 | 3266 | MQALQTP | Xiangyang Chi et al., 2020 |
| | | | | | | | | QT | (https://science.sciencemag. |
| | | | | | | | | | org/content/early/ |
| | | | | | | | | | 2020/06/19/science. |
| | | | | | | | | | abc6952/tab-pdf) |
| | | | | | | 0304-3H3 | 3267 | QQYNKWP | Xiangyang Chi et al., 2020 |
| | | | | | | | | PWT | (https://science.sciencemag. |
| | | | | | | | | | org/content/early/ |
| | | | | | | | | | 2020/06/19/science. |
| | | | | | | | | | abc6952/tab-pdf) |
| | | | | | | 0304- | 3268 | QQYYSSPY | Xiangyang Chi et al., 2020 |
| | | | | | | 4A10 | | | (https://science.sciencemag. |
| | | | | | | | | | org/content/early/ |
| | | | | | | | | | 2020/06/19/science. |
| | | | | | | | | | abc6952/tab-pdf) |
| | | | | | | 0304-4A2 | 3269 | HQYYNTPR | Xiangyang Chi et al., 2020 |
| | | | | | | | | T | (https://science.sciencemag. |
| | | | | | | | | | org/content/early/ |
| | | | | | | | | | 2020/06/19/science. |
| | | | | | | | | | abc6952/tab-pdf) |
| | | | | | | 0317-A1 | 3270 | RQRSNWP | Xiangyang Chi et al., 2020 |
| | | | | | | | | PT | (https://science.sciencemag. |
| | | | | | | | | | org/content/early/ |
| | | | | | | | | | 2020/06/19/science. |
| | | | | | | | | | abc6952/tab-pdf) |
| | | | | | | 0317-A2 | 3271 | QQSYSTPP | Xiangyang Chi et al., 2020 |
| | | | | | | | | T | (https://science.sciencemag. |
| | | | | | | | | | org/content/early/ |
| | | | | | | | | | 2020/06/19/science. |
| | | | | | | | | | abc6952/tab-pdf) |
| | | | | | | 0317-A3 | 3272 | QQTYRPP | Xiangyang Chi et al., 2020 |
| | | | | | | | | WT | (https://science.sciencemag. |
| | | | | | | | | | org/content/early/ |
| | | | | | | | | | 2020/06/19/science. |
| | | | | | | | | | abc6952/tab-pdf) |
| | | | | | | 0317-A7 | 3273 | MQTLQTP | Xiangyang Chi et al., 2020 |
| | | | | | | | | | (https://science.sciencemag. |
| | | | | | | | | | org/content/early/ |
| | | | | | | | | | 2020/06/19/science. |
| | | | | | | | | | abc6952/tab-pdf) |
| | | | | | | 0317-A8 | 3274 | QQYDNLPL | Xiangyang Chi et al., 2020 |
| | | | | | | | | T | (https://science.sciencemag. |
| | | | | | | | | | org/content/early/ |
| | | | | | | | | | 2020/06/19/science. |
| | | | | | | | | | abc6952/tab-pdf) |
| | | | | | | 0317-A9 | 3275 | MQATQFP | Xiangyang Chi et al., 2020 |
| | | | | | | | | | (https://science.sciencemag. |
| | | | | | | | | | org/content/early/ |
| | | | | | | | | | 2020/06/19/science. |
| | | | | | | | | | abc6952/tab-pdf) |
| | | | | | | 0317-B1 | 3276 | QQYYSTYG | Xiangyang Chi et al., 2020 |
| | | | | | | | | S | (https://science.sciencemag. |
| | | | | | | | | | org/content/early/ |
| | | | | | | | | | 2020/06/19/science. |
| | | | | | | | | | abc6952/tab-pdf) |
| | | | | | | 0317-C4 | 3277 | GTWDSSLS | Xiangyang Chi et al., 2020 |
| | | | | | | | | VW | (https://science.sciencemag. |
| | | | | | | | | | org/content/early/ |
| | | | | | | | | | 2020/06/19/science. |
| | | | | | | | | | abc6952/tab-pdf) |
| | | | | | | 0317-C9 | 3278 | AAWDDSL | Xiangyang Chi et al., 2020 |
| | | | | | | | | NGW | (https://science.sciencemag. |
| | | | | | | | | | org/content/early/ |
| | | | | | | | | | 2020/06/19/science. |
| | | | | | | | | | abc6952/tab-pdf) |
| | | | | | | 10C10 | 3279 | LQHNNYPL | Xiangyang Chi et al., 2020 |
| | | | | | | | | T | (https://science.sciencemag. |
| | | | | | | | | | org/content/early/ |
| | | | | | | | | | 2020/06/19/science. |
| | | | | | | | | | abc6952/tab-pdf) |
| | | | | | | 1A09 | 3280 | ALWYSNH | Wafaa Alsoussi et al., |
| | | | | | | | | WV | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1A10 | 3281 | QQGNTLP | Wafaa Alsoussi et al., |
| | | | | | | | | | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1A12 | 3282 | LQYDEFRT | Wafaa Alsoussi et al., |
| | | | | | | | | | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1B07 | 3283 | ALWYSNQ | Wafaa Alsoussi et al., |
| | | | | | | | | FI | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1610 | 3284 | ALWYSNH | Wafaa Alsoussi et al., |
| | | | | | | | | WV | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1C05 | 3285 | QQNNEDP | Wafaa Alsoussi et al., |
| | | | | | | | | LT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1C06 | 3286 | FQGSHVPP | Wafaa Alsoussi et al., |
| | | | | | | | | T | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1C07 | 3287 | QQNNEDP | Wafaa Alsoussi et al., |
| | | | | | | | | LT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1D04 | 3288 | LQYDEFRT | Wafaa Alsoussi et al., |
| | | | | | | | | | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1D05 | 3289 | ALWYSNQ | Wafaa Alsoussi et al., |
| | | | | | | | | FI | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1E02 | 3290 | QQNNEDP | Wafaa Alsoussi et al., |
| | | | | | | | | LT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1E07 | 3291 | QQSRKVP | Wafaa Alsoussi et al., |
| | | | | | | | | WT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1E10 | 3292 | QQWSSNP | Wafaa Alsoussi et al., |
| | | | | | | | | PT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1H06 | 3293 | QQSRKVP | Wafaa Alsoussi et al., |
| | | | | | | | | WT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1H10 | 3294 | ALWYSNH | Wafaa Alsoussi et al., |
| | | | | | | | | WV | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 1M-1D2 | 3295 | AAWDDSLI | Xiangyang Chi et al., 2020 |
| | | | | | | | | YVL | (https://science.sciencemag. |
| | | | | | | | | | org/content/early/ |
| | | | | | | | | | 2020/06/19/science. |
| | | | | | | | | | abc6952/tab-pdf) |
| | | | | | | 2B04 | 3296 | ALWYNNH | Wafaa Alsoussi et al., |
| | | | | | | | | WV | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2C02 | 3297 | QQWSSYP | Wafaa Alsoussi et al., |
| | | | | | | | | LT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2C03 | 3298 | FQGSHVPY | Wafaa Alsoussi et al., |
| | | | | | | | | T | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2C04 | 3299 | QQSRKVP | Wafaa Alsoussi et al., |
| | | | | | | | | WT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2D01 | 3300 | QQWSSYP | Wafaa Alsoussi et al., |
| | | | | | | | | LT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2D08 | 3301 | QQSRKVP | Wafaa Alsoussi et al., |
| | | | | | | | | WT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2D11 | 3302 | QQSKEVP | Wafaa Alsoussi et al., |
| | | | | | | | | WT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2E06 | 3303 | QQSNEDP | Wafaa Alsoussi et al., |
| | | | | | | | | | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2E10 | 3304 | QQNNEDP | Wafaa Alsoussi et al., |
| | | | | | | | | LT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2F04 | 3305 | QQSSSWP | Wafaa Alsoussi et al., |
| | | | | | | | | LT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2H04 | 3306 | QQSSSWP | Wafaa Alsoussi et al., |
| | | | | | | | | LT | 2020 |
| | | | | | | | | | (https://www.jimmunol. |
| | | | | | | | | | org/content/early/ |
| | | | | | | | | | 2020/06/23/jimmunol. |
| | | | | | | | | | 2000583) |
| | | | | | | 2M- | 3307 | QSYDSSNH | Xiangyang Chi et al., 2020 |
| | | | | | | 10B11 | | WV | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-12D7 | 3308 | QQSYSTPR | Xiangyang Chi et al., 2020 |
| | | | | | | | | T | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-13A3 | 3309 | QQYYSTPF | Xiangyang Chi et al., 2020 |
| | | | | | | | | T | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M- | 3310 | QQYGSSRS | Xiangyang Chi et al., 2020 |
| | | | | | | 13D11 | | WT | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-14B2 | 3311 | QQSYSTFT | Xiangyang Chi et al., 2020 |
| | | | | | | | | LYT | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-14E4 | 3312 | QQSHSFPF | Xiangyang Chi et al., 2020 |
| | | | | | | | | T | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-14E5 | 3313 | QQLNSYVT | Xiangyang Chi et al., 2020 |
| | | | | | | | | | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-2D1 | 3314 | AARDDSLS | Xiangyang Chi et al., 2020 |
| | | | | | | | | GWV | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-2D4 | 3315 | QQRTNWP | Xiangyang Chi et al., 2020 |
| | | | | | | | | L | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-2G12 | 3316 | QQNYSTW | Xiangyang Chi et al., 2020 |
| | | | | | | | | T | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-4G4 | 3317 | CSYAVSST | Xiangyang Chi et al., 2020 |
| | | | | | | | | WV | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-7E9 | 3318 | QQRSNWP | Xiangyang Chi et al., 2020 |
| | | | | | | | | PAFT | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-8E7 | 3319 | QQRSNWP | Xiangyang Chi et al., 2020 |
| | | | | | | | | PKIT | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-8H10 | 3320 | QQYYNNQ | Xiangyang Chi et al., 2020 |
| | | | | | | | | WT | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-9F10 | 3321 | QQSFVSPR | Xiangyang Chi et al., 2020 |
| | | | | | | | | T | (https://science.sciencemag. |
| | | | | | | | | | org/content/early/ |
| | | | | | | | | | 2020/06/19/science.abc6952/ |
| | | | | | | | | | tab-pdf) |
| | | | | | | 2M-9H1 | 3322 | LQHKSYPL | Xiangyang Chi et al., 2020 |
| | | | | | | | | T | (https://science.sciencemag. |
| | | | | | | | | | org/content/early/ |
| | | | | | | | | | 2020/06/19/science.abc6952/ |
| | | | | | | | | | tab-pdf) |
| +0O | | | | | | | | | |
| | | | | | | 31B5 | | ND | Xiangyu Chen et al., |
| | | | | | | | | | 2020 |
| | | | | | | | | | (https://www.nature.com/ |
| | | | | | | | | | articles/s41423-020- |
| | | | | | | | | | 0426-7) |
| | | | | | | 31B9 | | ND | Xiangyu Chen et al., |
| | | | | | | | | | 2020 |
| | | | | | | | | | (https://www.nature.com/ |
| | | | | | | | | | articles/s41423-020- |
| | | | | | | | | | 0426-7) |
| | | | | | | 32D4 | | ND | Xiangyu Chen et al., |
| | | | | | | | | | 2020 |
| | | | | | | | | | (https://www.nature.com/ |
| | | | | | | | | | articles/s41423-020- |
| | | | | | | | | | 0426-7) |
| | | | | | | 413-2 | 3323 | QQYGSSPP | Jinkai Wan et al., 2020 |
| | | | | | | | | LT | (https://www.sciencedirect. |
| | | | | | | | | | com/science/article/ |
| | | | | | | | | | pii/S2211124720308998) |
| | | | | | | 414-1 | 3324 | AAWDDSL | Jinkai Wan et al., 2020 |
| | | | | | | | | NGW | (https://www.sciencedirect. |
| | | | | | | | | | com/science/article/ |
| | | | | | | | | | pii/S2211124720308998) |
| | | | | | | 47D11 | | ND | Chunyan Wang et al., |
| | | | | | | | | | 2020 |
| | | | | | | | | | (https://www.nature.com/ |
| | | | | | | | | | articles/s41467-020- |
| | | | | | | | | | 16256-y) |
| | | | | | | 4A8 | 3325 | TQATQFPY | Xiangyang Chi et al., 2020 |
| | | | | | | | | T | (https://science.sciencemag. |
| | | | | | | | | | org/content/early/ |
| | | | | | | | | | 2020/06/19/science.abc6952/ |
| | | | | | | | | | tab-pdf) |
| | | | | | | 505-3 | 3326 | MQGTHW | Jinkai Wan et al., 2020 |
| | | | | | | | | PPT | (https://www.sciencedirect. |
| | | | | | | | | | com/science/article/ |
| | | | | | | | | | pii/S2211124720308998) |
| | | | | | | 505-5 | 3327 | MQGTHW | Jinkai Wan et al., 2020 |
| | | | | | | | | PPT | (https://www.sciencedirect. |
| | | | | | | | | | com/science/article/ |
| | | | | | | | | | pii/S2211124720308998) |
| | | | | | | 505-8 | 3328 | SSYTSSSIV | Jinkai Wan et al., 2020 |
| | | | | | | | | V | (https://www.sciencedirect. |
| | | | | | | | | | com/science/article/ |
| | | | | | | | | | pii/S2211124720308998) |
| | | | | | | 515-1 | 3329 | MQGTHW | Jinkai Wan et al., 2020 |
| | | | | | | | | PPT | (https://www.sciencedirect. |
| | | | | | | | | | com/science/article/ |
| | | | | | | | | | pii/S2211124720308998) |
| | | | | | | 515-5 | 3330 | LQHNSYPI | Jinkai Wan et al., 2020 |
| | | | | | | | | T | (https://www.sciencedirect. |
| | | | | | | | | | com/science/article/ |
| | | | | | | | | | pii/S2211124720308998) |
| | | | | | | 553-15 | 3331 | QSYDGSN | Jinkai Wan et al., 2020 |
| | | | | | | | | HNVV | (https://www.sciencedirect. |
| | | | | | | | | | com/science/article/ |
| | | | | | | | | | pii/S2211124720308998) |
| | | | | | | 553-49 | 3332 | QSYDSSNH | Jinkai Wan et al., 2020 |
| | | | | | | | | W | (https://www.sciencedirect. |
| | | | | | | | | | com/science/article/ |
| | | | | | | | | | pii/S2211124720308998) |
| | | | | | | 553-60 | 3333 | QQYGSSPP | Jinkai Wan et al., 2020 |
| | | | | | | | | T | (https://www.sciencedirect. |
| | | | | | | | | | com/science/article/ |
| | | | | | | | | | pii/S2211124720308998) |
| | | | | | | 553-63 | 3334 | QQSYSIPR | Jinkai Wan et al., 2020 |
| | | | | | | | | T | (https://www.sciencedirect. |
| | | | | | | | | | com/science/article/ |
| | | | | | | | | | pii/S2211124720308998) |
| | | | | | | 8D2 | 3335 | LQHNSYPL | Xiangyang Chi et al., 2020 |
| | | | | | | | | T | (https://science.sciencemag. |
| | | | | | | | | | org/content/early/ |
| | | | | | | | | | 2020/06/19/science.abc |
| | | | | | | | | | 6952/tab-pdf) |
| | | | | | | 8D9 | 3336 | QAWDSST | Xiangyang Chi et al., 2020 |
| | | | | | | | | GV | (https://science.sciencemag. |
| | | | | | | | | | org/content/early/ |
| | | | | | | | | | 2020/06/19/science.abc |
| | | | | | | | | | 6952/tab-pdf) |
| | | | | | | 9A1 | 3337 | HQYSKWP | Xiangyang Chi et al., 2020 |
| | | | | | | | | VT | (https://science.sciencemag. |
| | | | | | | | | | org/content/early/ |
| | | | | | | | | | 2020/06/19/science.abc |
| | | | | | | | | | 6952/tab-pdf) |
| | | | | | | Ab_510A | 3338 | MQALQM | Xiaojian Han et al., 2020 |
| | | | | | | | | GT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_510A5 | 3339 | QQSYSTPP | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_510D7 | 3340 | QQYGSSRT | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_510G | 3341 | QQYDTLPL | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_510H10 | 3342 | QQSYSTPR | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_510H2 | 3343 | QQLNSYPR | Xiaojian Han et al., 2020 |
| | | | | | | | | MT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_510H | 3344 | QQRSNWP | Xiaojian Han et al., 2020 |
| | | | | | | | | GT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_510H7 | 3345 | QQFNNFLL | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511A1 | 3346 | QQYNNWP | Xiaojian Han et al., 2020 |
| | | | | | | | | PWT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511A5 | 3347 | QVWDSSA | Xiaojian Han et al., 2020 |
| | | | | | | | | DHYV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511B11 | 3348 | QSYDRSLS | Xiaojian Han et al., 2020 |
| | | | | | | | | VLYV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511B | 3349 | QQANSFRL | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511D11 | 3350 | QQYGRSPY | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511E5 | 3351 | NSRDSSGN | Xiaojian Han et al., 2020 |
| | | | | | | | | TVV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511E7 | 3352 | QQYHNLPI | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511E9 | 3353 | HQSSSLPY | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511G5 | 3354 | TTWDASR | Xiaojian Han et al., 2020 |
| | | | | | | | | GGWV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511G7 | 3355 | QQYHNVP | Xiaojian Han et al., 2020 |
| | | | | | | | | PA | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511H11 | 3356 | QQSYSSPP | Xiaojian Han et al., 2020 |
| | | | | | | | | WT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_511H7 | 3357 | AAWDDSL | Xiaojian Han et al., 2020 |
| | | | | | | | | SGPV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51A1 | 3358 | QHLNIDPIT | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51A3 | 3359 | QQRGNSIT | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51D2 | 3360 | QQSYNTLA | Xiaojian Han et al., 2020 |
| | | | | | | | | LS | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51D3 | 3361 | QQYADLPY | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51D4 | 3362 | QQSYSPPH | Xiaojian Han et al., 2020 |
| | | | | | | | | S | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51D7 | 3363 | QQSYSTPC | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51E10 | 3364 | QQYYITPQ | Xiaojian Han et al., 2020 |
| | | | | | | | | LT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51E12 | 3365 | QQYYGYPT | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51E7 | 3366 | QQSYSAPP | Xiaojian Han et al., 2020 |
| | | | | | | | | WT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_51F11 | 3367 | QQYGSSRT | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_52C1 | 3368 | QSYDSSLS | Xiaojian Han et al., 2020 |
| | | | | | | | | GSWV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_52C6 | 3369 | VLYMGSGI | Xiaojian Han et al., 2020 |
| | | | | | | | | W | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_52F7 | 3370 | MQGTHW | Xiaojian Han et al., 2020 |
| | | | | | | | | PPGT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_52G9 | 3371 | QQYDNLP | Xiaojian Han et al., 2020 |
| | | | | | | | | RT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_53C10 | 3372 | QVWDSSS | Xiaojian Han et al., 2020 |
| | | | | | | | | DPYV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_53C5 | 3373 | QQYNNLYT | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_53F12 | 3374 | QHYNNWP | Xiaojian Han et al., 2020 |
| | | | | | | | | LYT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_53F9 | 3375 | MQGTHW | Xiaojian Han et al., 2020 |
| | | | | | | | | PPGT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_53H3 | 3376 | QQYDNLPL | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_55A8 | 3377 | QQYNSYSH | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_55C9 | 3378 | QQSYSTPP | Xiaojian Han et al., 2020 |
| | | | | | | | | YT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_56C12 | 3379 | QQTYSTPR | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_56D7 | 3380 | QQLNSYPP | Xiaojian Han et al., 2020 |
| | | | | | | | | IT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_56E1 | 3381 | AAWDDSL | Xiaojian Han et al., 2020 |
| | | | | | | | | NGWV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_56H11 | 3382 | ATWDDSL | Xiaojian Han et al., 2020 |
| | | | | | | | | NGRV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_56H3 | 3383 | QQYGSSPR | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_57A6 | 3384 | NSRDSSGN | Xiaojian Han et al., 2020 |
| | | | | | | | | HWV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_57A8 | 3385 | QQYNSYSP | Xiaojian Han et al., 2020 |
| | | | | | | | | LT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_57A9 | 3386 | QQYYSFW | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_57B8 | 3387 | QLLNTDPI | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_57C4 | 3388 | QQSYSTPL | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_57E11 | 3389 | AAWDDSL | Xiaojian Han et al., 2020 |
| | | | | | | | | NGWV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_57F7 | 3390 | AVWDDSL | Xiaojian Han et al., 2020 |
| | | | | | | | | NGWV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_57G9 | 3391 | QQSYSIPR | Xiaojian Han et al., 2020 |
| | | | | | | | | T | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_58A4 | 3392 | QQYGSSP | Xiaojian Han et al., 2020 |
| | | | | | | | | WT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_58D2 | 3393 | QQLGT | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_58G1 | 3394 | QQTNSFPT | Xiaojian Han et al., 2020 |
| | | | | | | | | | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_58G6 | 3395 | QQYDNSP | Xiaojian Han et al., 2020 |
| | | | | | | | | WT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_59A2 | 3396 | QQSDNVP | Xiaojian Han et al., 2020 |
| | | | | | | | | VT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_59D6 | 3397 | QSYDSALV | Xiaojian Han et al., 2020 |
| | | | | | | | | V | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_81A11 | 3398 | QSYDSSLS | Xiaojian Han et al., 2020 |
| | | | | | | | | VW | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_81C3 | 3399 | SSFTSSSTP | Xiaojian Han et al., 2020 |
| | | | | | | | | YV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_81C7 | 3400 | AAWDDSL | Xiaojian Han et al., 2020 |
| | | | | | | | | NGPV | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_81C8 | 3401 | QQSYNTPP | Xiaojian Han et al., 2020 |
| | | | | | | | | WT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_81E1 | 3402 | AAWDDSL | Xiaojian Han et al., 2020 |
| | | | | | | | | SRW | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_81E10 | 3403 | AAWDDSL | Xiaojian Han et al., 2020 |
| | | | | | | | | SGVI | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_81F2 | 3404 | MQALQTP | Xiaojian Han et al., 2020 |
| | | | | | | | | RT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_82B6 | 3405 | CMQGTH | Xiaojian Han et al., 2020 |
| | | | | | | | | WPPT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_82C6 | 3406 | MQGTHW | Xiaojian Han et al., 2020 |
| | | | | | | | | PMYT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Ab_82F6 | 3407 | QQHDNVP | Xiaojian Han et al., 2020 |
| | | | | | | | | LT | (https://www.biorxiv. |
| | | | | | | | | | org/content/10.1101/ |
| | | | | | | | | | 2020.08.19.253369v2.full. |
| | | | | | | | | | pdf + html) |
| | | | | | | Acharya | | Various | Priyamvada Acharya et |
| | | | | | | et al., | | | al., 2020 |
| | | | | | | 2020 | | | (https://www.biorxiv.or |
| | | | | | | | | | g/content/10.1101/ |
| | | | | | | | | | 2020.06.30.178897v1); Wilton |
| | | | | | | | | | Williams et al., 2020 |
| | | | | | | | | | (https://www.biorxiv. |
| | | | | | | | | | org/content/10.1101/ |
| | | | | | | | | | 2020.06.30.178921V1) |
| | | | | | | B38 | 3408 | QQLNSYPP | Yan Wu et al., 2020 |
| | | | | | | | | | (https://science.sciencemag. |
| | | | | | | | | | org/content/early/ |
| | | | | | | | | | 2020/05/12/science.abc2241) |
| | | | | | | BD-236 | | ND | Shuo Du et al., 2020 |
| | | | | | | | | | (https://www.biorxiv. |
| | | | | | | | | | org/content/10.1101/ |
| | | | | | | | | | 2020.07.09.195263v1) |
| | | | | | | BD-368-2 | | ND | Shuo Du et al., 2020 |
| | | | | | | | | | (https://www.biorxiv. |
| | | | | | | | | | org/content/10.1101/ |
| | | | | | | | | | 2020.07.09.195263v1) |
| | | | | | | BD-494 | 3409 | QQLNSYPF | Yunlong Cao et al., 2020 |
| | | | | | | | | T | (https://www.sciencedirect. |
| | | | | | | | | | com/science/article/ |
| | | | | | | | | | pii/S0092867420306206) |
| | | | | | | BD-498 | 3410 | QQLNSYPL | Yunlong Cao et al., 2020 |
| | | | | | | | | T | (https://www.sciencedirect. |
| | | | | | | | | | com/science/article/ |
| | | | | | | | | | pii/S0092867420306206) |
| | | | | | | BD-500 | 3411 | QQSYSTPP | Yunlong Cao et al., 2020 |
| | | | | | | | | DT | (https://www.sciencedirect. |
| | | | | | | | | | com/science/article/ |
| | | | | | | | | | pii/S0092867420306206) |
| | | | | | | BD-503 | 3412 | QQSYTTPL | Yunlong Cao et al., 2020 |
| | | | | | | | | | (https://www.sciencedirect. |
| | | | | | | | | | com/science/article/ |
| | | | | | | | | | pii/S0092867420306206) |
| | | | | | | BD-504 | 3413 | QQSYTTPL | Yunlong Cao et al., 2020 |
| | | | | | | | | | (https://www.sciencedirect. |
| | | | | | | | | | com/science/article/ |
| | | | | | | | | | pii/S0092867420306206) |
| | | | | | | BD-505 | 3414 | HQYDNLPP | Yunlong Cao et al., 2020 |
| | | | | | | | | T | (https://www.sciencedirect. |
| | | | | | | | | | com/science/article/ |
| | | | | | | | | | pii/S0092867420306206) |
| | | | | | | BD-506 | 3415 | QQLNSYPL | Yunlong Cao et al., 2020 |
| | | | | | | | | T | (https://www.sciencedirect. |
| | | | | | | | | | com/science/article/ |
| | | | | | | | | | pii/S0092867420306206) |
| | | | | | | BD-507 | 3416 | QQLNSNP | Yunlong Cao et al., 2020 |
| | | | | | | | | PIT | (https://www.sciencedirect. |
| | | | | | | | | | com/science/article/ |
| | | | | | | | | | pii/S0092867420306206) |
| | | | | | | BD-508 | 3417 | QQSYSTPP | Yunlong Cao et al., 2020 |
| | | | | | | | | | (https://www.sciencedirect. |
| | | | | | | | | | com/science/article/ |
| | | | | | | | | | pii/S0092867420306206) |
| | | | | | | BD-515 | | ND | Yunlong Cao et al., 2020 |
| | | | | | | | | | (https://www.sciencedirect. |
| | | | | | | | | | com/science/article/ |
| | | | | | | | | | pii/S0092867420306206) |
| | | | | | | BD-604 | | ND | Shuo Du et al., 2020 |
| | | | | | | | | | (https://www.biorxiv.or |
| | | | | | | | | | g/content/10.1101/ |
| | | | | | | | | | 2020.07.09.195263vl) |
| | | | | | | BD-629 | | ND | Shuo Du et al., 2020 |
| | | | | | | | | | (https://www.biorxiv.or |
| | | | | | | | | | g/content/10.1101/ |
| | | | | | | | | | 2020.07.09.195263V1) |
| | | | | | | BD23 | 3418 | QQYNSYPY | Yunlong Cao et al., 2020 |
| | | | | | | | | T | (https://www.sciencedirect. |
| | | | | | | | | | com/science/article/ |
| | | | | | | | | | pii/S0092867420306206) |
| | | | | | | Bertoglio | | Various | Federico Bertoglio et |
| | | | | | | et al., | | | al., 2020 |
| | | | | | | 2020 | | | (https://www.biorxiv. |
| | | | | | | | | | org/content/10.1101/ |
| | | | | | | | | | 2020.06.05.135921V1) |
| | | | | | | C002 | 3419 | QQSYSTPR | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C003 | 3420 | QQYGSSPR | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C004 | 3421 | QQYDNLPI | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C005 | 3422 | QQYGSSP | Davide Robbiani et al., |
| | | | | | | | | WT | 2020 |
| | | | | | | | | | (https://www.nature.com/ |
| | | | | | | | | | articles/s41586-020- |
| | | | | | | | | | 2456-9) |
| | | | | | | C006 | 3423 | AAWDDSL | Davide Robbiani et al., |
| | | | | | | | | NGPV | 2020 |
| | | | | | | | | | (https://www.nature.com/ |
| | | | | | | | | | articles/s41586-020- |
| | | | | | | | | | 2456-9) |
| | | | | | | C008 | 3424 | QQYNSYW | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C009 | 3425 | SSDAGSNN | Davide Robbiani et al., |
| | | | | | | | | W | 2020 |
| | | | | | | | | | (https://www.nature.com/ |
| | | | | | | | | | articles/s41586-020- |
| | | | | | | | | | 2456-9) |
| | | | | | | C010 | 3426 | QQSYSTPP | Davide Robbiani et al., |
| | | | | | | | | WT | 2020 |
| | | | | | | | | | (https://www.nature.com/ |
| | | | | | | | | | articles/s41586-020- |
| | | | | | | | | | 2456-9) |
| | | | | | | C013 | 3427 | QQRSNWP | Davide Robbiani et al., |
| | | | | | | | | LT | 2020 |
| | | | | | | | | | (https://www.nature.com/ |
| | | | | | | | | | articles/s41586-020- |
| | | | | | | | | | 2456-9) |
| | | | | | | C016 | 3428 | QQYDNLP | Davide Robbiani et al., |
| | | | | | | | | PT | 2020 |
| | | | | | | | | | (https://www.nature.com/ |
| | | | | | | | | | articles/s41586-020- |
| | | | | | | | | | 2456-9) |
| | | | | | | C017 | 3429 | QQRIT | Davide Robbiani et al., |
| | | | | | | | | | 2020 |
| | | | | | | | | | (https://www.nature.com/ |
| | | | | | | | | | articles/s41586-020- |
| | | | | | | | | | 2456-9) |
| | | | | | | C018 | 3430 | QQSYSTPP | Davide Robbiani et al., |
| | | | | | | | | AT | 2020 |
| | | | | | | | | | (https://www.nature.com/ |
| | | | | | | | | | articles/s41586-020- |
| | | | | | | | | | 2456-9) |
| | | | | | | C019 | 3431 | QVWDSSS | Davide Robbiani et al., |
| | | | | | | | | DHVV | 2020 |
| | | | | | | | | | (https://www.nature.com/ |
| | | | | | | | | | articles/s41586-020- |
| | | | | | | | | | 2456-9) |
| | | | | | | C021 | 3432 | MQALQTP | Davide Robbiani et al., |
| | | | | | | | | | 2020 |
| | | | | | | | | | (https://www.nature.com/ |
| | | | | | | | | | articles/s41586-020- |
| | | | | | | | | | 2456-9) |
| | | | | | | C022 | 3433 | QQYNNYR | Davide Robbiani et al., |
| | | | | | | | | | 2020 |
| | | | | | | | | | (https://www.nature.com/ |
| | | | | | | | | | articles/s41586-020- |
| | | | | | | | | | 2456-9) |
| | | | | | | C027 | 3434 | QQYNSYST | Davide Robbiani et al., |
| | | | | | | | | | 2020 |
| | | | | | | | | | (https://www.nature.com/ |
| | | | | | | | | | articles/s41586-020- |
| | | | | | | | | | 2456-9) |
| | | | | | | C029 | 3435 | MQALQTP | Davide Robbiani et al., |
| | | | | | | | | HT | 2020 |
| | | | | | | | | | (https://www.nature.com/ |
| | | | | | | | | | articles/s41586-020- |
| | | | | | | | | | 2456-9) |
| | | | | | | C030 | 3436 | QQYNSYST | Davide Robbiani et al., |
| | | | | | | | | | 2020 |
| | | | | | | | | | (https://www.nature.com/ |
| | | | | | | | | | articles/s41586-020- |
| | | | | | | | | | 2456-9) |
| | | | | | | C031 | 3437 | QQSYSTPP | Davide Robbiani et al., |
| | | | | | | | | LT | 2020 |
| | | | | | | | | | (https://www.nature.com/ |
| | | | | | | | | | articles/s41586-020- |
| | | | | | | | | | 2456-9) |
| | | | | | | C101 | 3438 | QQYGSSPR | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C102 | 3439 | QQYGSSPR | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C103 | 3440 | QQYTTTPR | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C104 | 3441 | QQYGTTPR | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C105 | 3442 | SSYEGSNN | Davide Robbiani et al., |
| | | | | | | | | FVV | 2020 |
| | | | | | | | | | (https://www.nature.com/ |
| | | | | | | | | | articles/s41586-020- |
| | | | | | | | | | 2456-9); Christopher |
| | | | | | | | | | Barnes et al., 2020 |
| | | | | | | | | | (https://www.sciencedirect. |
| | | | | | | | | | com/science/article/ |
| | | | | | | | | | pii/S0092867420307571) |
| | | | | | | C106 | 3443 | QVWDSSR | Davide Robbiani et al., |
| | | | | | | | | DHVV | 2020 |
| | | | | | | | | | (https://www.nature.com/ |
| | | | | | | | | | articles/s41586-020- |
| | | | | | | | | | 2456-9) |
| | | | | | | C107 | 3444 | AAWDDSL | Davide Robbiani et al., |
| | | | | | | | | SGFW | 2020 |
| | | | | | | | | | (https://www.nature.com/ |
| | | | | | | | | | articles/s41586-020- |
| | | | | | | | | | 2456-9) |
| | | | | | | C108 | 3445 | NSYTSSSTR | Davide Robbiani et al., |
| | | | | | | | | V | 2020 |
| | | | | | | | | | (https://www.nature.com/ |
| | | | | | | | | | articles/s41586-020- |
| | | | | | | | | | 2456-9) |
| | | | | | | C109 | 3446 | SSYAGSNN | Davide Robbiani et al., |
| | | | | | | | | YVV | 2020 |
| | | | | | | | | | (https://www.nature.com/ |
| | | | | | | | | | articles/s41586-020- |
| | | | | | | | | | 2456-9) |
| | | | | | | C110 | 3447 | QQYNSYPY | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C111 | 3448 | AAWDDSL | Davide Robbiani et al., |
| | | | | | | | | NGAWV | 2020 |
| | | | | | | | | | (https://www.nature.com/ |
| | | | | | | | | | articles/s41586-020- |
| | | | | | | | | | 2456-9) |
| | | | | | | C112 | 3449 | SSYTSSST | Davide Robbiani et al., |
| | | | | | | | | WV | 2020 |
| | | | | | | | | | (https://www.nature.com/ |
| | | | | | | | | | articles/s41586-020- |
| | | | | | | | | | 2456-9) |
| | | | | | | C113 | 3450 | QQHNSSPL | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C114 | 3451 | QSYDSSLY | Davide Robbiani et al., |
| | | | | | | | | AV | 2020 |
| | | | | | | | | | (https://www.nature.com/ |
| | | | | | | | | | articles/s41586-020- |
| | | | | | | | | | 2456-9) |
| | | | | | | C115 | 3452 | MQVLQIPY | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C116 | 3453 | QSYDSGN | Davide Robbiani et al., |
| | | | | | | | | HWVV | 2020 |
| | | | | | | | | | (https://www.nature.com/ |
| | | | | | | | | | articles/s41586-020- |
| | | | | | | | | | 2456-9) |
| | | | | | | C117 | 3454 | GAWDSSLS | Davide Robbiani et al., |
| | | | | | | | | AGGVYV | 2020 |
| | | | | | | | | | (https://www.nature.com/ |
| | | | | | | | | | articles/s41586-020- |
| | | | | | | | | | 2456-9) |
| | | | | | | C118 | 3455 | QTWGTGIL | Davide Robbiani et al., |
| | | | | | | | | V | 2020 |
| | | | | | | | | | (https://www.nature.com/ |
| | | | | | | | | | articles/s41586-020- |
| | | | | | | | | | 2456-9) |
| | | | | | | C119 | 3456 | SSYTSSSTS | Davide Robbiani et al., |
| | | | | | | | | W | 2020 |
| | | | | | | | | | (https://www.nature.com/ |
| | | | | | | | | | articles/s41586-020- |
| | | | | | | | | | 2456-9) |
| | | | | | | C120 | 3457 | QQYDNLP | Davide Robbiani et al., |
| | | | | | | | | QT | 2020 |
| | | | | | | | | | (https://www.nature.com/ |
| | | | | | | | | | articles/s41586-020- |
| | | | | | | | | | 2456-9) |
| | | | | | | C121 | 3458 | CSYAGSSTL | Davide Robbiani et al., |
| | | | | | | | | V | 2020 |
| | | | | | | | | | (https://www.nature.com/ |
| | | | | | | | | | articles/s41586-020- |
| | | | | | | | | | 2456-9) |
| | | | | | | C122 | 3459 | QQLNSDSY | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C123 | 3460 | QQLNSYPP | Davide Robbiani et al., |
| | | | | | | | | | 2020 |
| | | | | | | | | | (https://www.nature.com/ |
| | | | | | | | | | articles/s41586-020- |
| | | | | | | | | | 2456-9) |
| | | | | | | C124 | 3461 | QQRNNW | Davide Robbiani et al., |
| | | | | | | | | PPEWT | 2020 |
| | | | | | | | | | (https://www.nature.com/ |
| | | | | | | | | | articles/s41586-020- |
| | | | | | | | | | 2456-9) |
| | | | | | | C125 | 3462 | QQYGSSP | Davide Robbiani et al., |
| | | | | | | | | WT | 2020 |
| | | | | | | | | | (https://www.nature.com/ |
| | | | | | | | | | articles/s41586-020- |
| | | | | | | | | | 2456-9) |
| | | | | | | C126 | 3463 | QSYDSSNL | Davide Robbiani et al., |
| | | | | | | | | V | 2020 |
| | | | | | | | | | (https://www.nature.com/ |
| | | | | | | | | | articles/s41586-020- |
| | | | | | | | | | 2456-9) |
| | | | | | | C127 | 3464 | AAWDDSL | Davide Robbiani et al., |
| | | | | | | | | NGW | 2020 |
| | | | | | | | | | (https://www.nature.com/ |
| | | | | | | | | | articles/s41586-020- |
| | | | | | | | | | 2456-9) |
| | | | | | | C128 | 3465 | QQYGSSRA | Davide Robbiani et al., |
| | | | | | | | | LT | 2020 |
| | | | | | | | | | (https://www.nature.com/ |
| | | | | | | | | | articles/s41586-020- |
| | | | | | | | | | 2456-9) |
| | | | | | | C130 | 3466 | QVWDSSS | Davide Robbiani et al., |
| | | | | | | | | DHPGVV | 2020 |
| | | | | | | | | | (https://www.nature.com/ |
| | | | | | | | | | articles/s41586-020- |
| | | | | | | | | | 2456-9) |
| | | | | | | C131 | 3467 | QQYNNWP | Davide Robbiani et al., |
| | | | | | | | | IT | 2020 |
| | | | | | | | | | (https://www.nature.com/ |
| | | | | | | | | | articles/s41586-020- |
| | | | | | | | | | 2456-9) |
| | | | | | | C132 | 3468 | SSYTSSSTL | Davide Robbiani et al., |
| | | | | | | | | L | 2020 |
| | | | | | | | | | (https://www.nature.com/ |
| | | | | | | | | | articles/s41586-020- |
| | | | | | | | | | 2456-9) |
| | | | | | | C133 | 3469 | QQSYSTPP | Davide Robbiani et al., |
| | | | | | | | | WT | 2020 |
| | | | | | | | | | (https://www.nature.com/ |
| | | | | | | | | | articles/s41586-020- |
| | | | | | | | | | 2456-9) |
| | | | | | | C134 | 3470 | QVWDSSS | Davide Robbiani et al., |
| | | | | | | | | DRPGVV | 2020 |
| | | | | | | | | | (https://www.nature.com/ |
| | | | | | | | | | articles/s41586-020- |
| | | | | | | | | | 2456-9) |
| | | | | | | C135 | 3471 | QQYNSYP | Davide Robbiani et al., |
| | | | | | | | | WT | 2020 |
| | | | | | | | | | (https://www.nature.com/ |
| | | | | | | | | | articles/s41586-020- |
| | | | | | | | | | 2456-9) |
| | | | | | | C138 | 3472 | QSYDSSN | Davide Robbiani et al., |
| | | | | | | | | WV | 2020 |
| | | | | | | | | | (https://www.nature.com/ |
| | | | | | | | | | articles/s41586-020- |
| | | | | | | | | | 2456-9) |
| | | | | | | C139 | 3473 | QQYDNLPL | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C140 | 3474 | QQLNSYSY | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C141 | 3475 | QSYDSSN | Davide Robbiani et al., |
| | | | | | | | | WV | 2020 |
| | | | | | | | | | (https://www.nature.com/ |
| | | | | | | | | | articles/s41586-020- |
| | | | | | | | | | 2456-9) |
| | | | | | | C143 | 3476 | CSYAGAST | Davide Robbiani et al., |
| | | | | | | | | FV | 2020 |
| | | | | | | | | | (https://www.nature.com/ |
| | | | | | | | | | articles/s41586-020- |
| | | | | | | | | | 2456-9) |
| | | | | | | C144 | 3477 | SSYTSSSTR | Davide Robbiani et al., |
| | | | | | | | | V | 2020 |
| | | | | | | | | | (https://www.nature.com/ |
| | | | | | | | | | articles/s41586-020- |
| | | | | | | | | | 2456-9) |
| | | | | | | C145 | 3478 | SSYTSSTTR | Davide Robbiani et al., |
| | | | | | | | | V | 2020 |
| | | | | | | | | | (https://www.nature.com/ |
| | | | | | | | | | articles/s41586-020- |
| | | | | | | | | | 2456-9) |
| | | | | | | C146 | 3479 | SSYRGSSTP | Davide Robbiani et al., |
| | | | | | | | | YV | 2020 |
| | | | | | | | | | (https://www.nature.com/ |
| | | | | | | | | | articles/s41586-020- |
| | | | | | | | | | 2456-9) |
| | | | | | | C147 | 3480 | LLSYSGAR | Davide Robbiani et al., |
| | | | | | | | | PV | 2020 |
| | | | | | | | | | (https://www.nature.com/ |
| | | | | | | | | | articles/s41586-020- |
| | | | | | | | | | 2456-9) |
| | | | | | | C148 | 3481 | QQYNNWP | Davide Robbiani et al., |
| | | | | | | | | PLT | 2020 |
| | | | | | | | | | (https://www.nature.com/ |
| | | | | | | | | | articles/s41586-020- |
| | | | | | | | | | 2456-9) |
| | | | | | | C150 | 3482 | SSYRSSSTL | Davide Robbiani et al., |
| | | | | | | | | V | 2020 |
| | | | | | | | | | (https://www.nature.com/ |
| | | | | | | | | | articles/s41586-020- |
| | | | | | | | | | 2456-9) |
| | | | | | | C151 | 3483 | QSYDSSNY | Davide Robbiani et al., |
| | | | | | | | | WV | 2020 |
| | | | | | | | | | (https://www.nature.com/ |
| | | | | | | | | | articles/s41586-020- |
| | | | | | | | | | 2456-9) |
| | | | | | | C153 | 3484 | CSYAGSST | Davide Robbiani et al., |
| | | | | | | | | WV | 2020 |
| | | | | | | | | | (https://www.nature.com/ |
| | | | | | | | | | articles/s41586-020- |
| | | | | | | | | | 2456-9) |
| | | | | | | C154 | 3485 | QQYDNLPI | Davide Robbiani et al., |
| | | | | | | | | T | 2020 |
| | | | | | | | | | (https://www.nature.com/ |
| | | | | | | | | | articles/s41586-020- |
| | | | | | | | | | 2456-9) |
| | | | | | | C155 | 3486 | QQYNNWP | Davide Robbiani et al., |
| | | | | | | | | RT | 2020 |
| | | | | | | | | | (https://www.nature.com/ |
| | | | | | | | | | articles/s41586-020- |
| | | | | | | | | | 2456-9) |
| | | | | | | C156 | 3487 | QVWDSSS | Davide Robbiani et al., |
| | | | | | | | | DPWV | 2020 |
| | | | | | | | | | (https://www.nature.com/ |
| | | | | | | | | | articles/s41586-020- |
| | | | | | | | | | 2456-9) |
| | | | | | | C164 | 3488 | CSYAGAST | Davide Robbiani et al., |
| | | | | | | | | FV | 2020 |
| | | | | | | | | | (https://www.nature.com/ |
| | | | | | | | | | articles/s41586-020- |
| | | | | | | | | | 2456-9) |
| | | | | | | C165 | 3489 | QQYGSSP | Davide Robbiani et al., |
| | | | | | | | | WT | 2020 |
| | | | | | | | | | (https://www.biorxiv. |
| | | | | | | | | | org/content/10.1101/ |
| | | | | | | | | | 2020.05.13.092619v2) |
| | | | | | | C201 | 3490 | QQANSFPL | Davide Robbiani et al., |
| | | | | | | | | T | 2020 |
| | | | | | | | | | (https://www.biorxiv. |
| | | | | | | | | | org/content/10.1101/ |
| | | | | | | | | | 2020.05.13.092619v2) |
| | | | | | | C202 | 3491 | QQYDNLP | Davide Robbiani et al., |
| | | | | | | | | RS | 2020 |
| | | | | | | | | | (https://www.biorxiv. |
| | | | | | | | | | org/content/10.1101/ |
| | | | | | | | | | 2020.05.13.092619v2) |
| | | | | | | C204 | 3492 | QQSYSTPP | Davide Robbiani et al., |
| | | | | | | | | WT | 2020 |
| | | | | | | | | | (https://www.biorxiv. |
| | | | | | | | | | org/content/10.1101/ |
| | | | | | | | | | 2020.05.13.092619v2) |
| | | | | | | C205 | 3493 | QQYVSSP | Davide Robbiani et al., |
| | | | | | | | | WT | 2020 |
| | | | | | | | | | (https://www.biorxiv. |
| | | | | | | | | | org/content/10.1101/ |
| | | | | | | | | | 2020.05.13.092619v2) |
| | | | | | | C207 | 3494 | QQRSNWP | Davide Robbiani et al., |
| | | | | | | | | RG | 2020 |
| | | | | | | | | | (https://www.biorxiv. |
| | | | | | | | | | org/content/10.1101/ |
| | | | | | | | | | 2020.05.13.092619v2) |
| | | | | | | C208 | 3495 | QQYGSSLT | Davide Robbiani et al., |
| | | | | | | | | | 2020 |
| | | | | | | | | | (https://www.biorxiv. |
| | | | | | | | | | org/content/10.1101/ |
| | | | | | | | | | 2020.05.13.092619v2) |
| | | | | | | C210 | 3496 | QQLNSYP | Davide Robbiani et al., |
| | | | | | | | | QGT | 2020 |
| | | | | | | | | | (https://www.biorxiv. |
| | | | | | | | | | org/content/10.1101/ |
| | | | | | | | | | 2020.05.13.092619v2) |
| | | | | | | C211 | 3497 | QQYNNWP | Davide Robbiani et al., |
| | | | | | | | | RT | 2020 |
| | | | | | | | | | (https://www.biorxiv. |
| | | | | | | | | | org/content/10.1101/ |
| | | | | | | | | | 2020.05.13.092619v2) |
| | | | | | | C212 | 3498 | CSYAGSST | Davide Robbiani et al., |
| | | | | | | | | RL | 2020 |
| | | | | | | | | | (https://www.biorxiv. |
| | | | | | | | | | org/content/10.1101/ |
| | | | | | | | | | 2020.05.13.092619v2) |
| | | | | | | C214 | 3499 | QQSYSTPP | Davide Robbiani et al., |
| | | | | | | | | WT | 2020 |
| | | | | | | | | | (https://www.biorxiv. |
| | | | | | | | | | org/content/10.1101/ |
| | | | | | | | | | 2020.05.13.092619v2) |
| | | | | | | C215 | 3500 | QQSYSTPP | Davide Robbiani et al., |
| | | | | | | | | WT | 2020 |
| | | | | | | | | | (https://www.biorxiv. |
| | | | | | | | | | org/content/10.1101/ |
| | | | | | | | | | 2020.05.13.092619v2) |
| | | | | | | C216 | 3501 | QQSYSTPPI | Davide Robbiani et al., |
| | | | | | | | | T | 2020 |
| | | | | | | | | | (https://www.biorxiv. |
| | | | | | | | | | org/content/10.1101/ |
| | | | | | | | | | 2020.05.13.092619v2) |
| | | | | | | CA1 | 3502 | QQRRNW | Rui Shi et al., 2020 |
| | | | | | | | | GT | (https://www.nature.com/ |
| | | | | | | | | | articles/s41586-020- |
| | | | | | | | | | 2381- |
| | | | | | | | | | y_reference.pdf); NCBI |
| | | | | | | | | | (https://www.ncbi.nlm. |
| | | | | | | | | | nih.gov/nuccore/MT470194); |
| | | | | | | | | | NCBI |
| | | | | | | | | | (https://www.ncbi.nlm. |
| | | | | | | | | | nih.gov/nuccore/MT4701945) |
| | | | | | | CB6 | 3503 | QQSYSTPP | Rui Shi et al., 2020 |
| | | | | | | | | EYT | (https://www.nature.com/ |
| | | | | | | | | | articles/s41586-020- |
| | | | | | | | | | 2381- |
| | | | | | | | | | y_reference.pdf); NCBI |
| | | | | | | | | | (https://www.ncbi.nlm. |
| | | | | | | | | | nih.gov/nuccore/MT470 |
| | | | | | | | | | 194);NCBI |
| | | | | | | | | | (https://www.ncbi.nlm. |
| | | | | | | | | | nih.gov/nuccore/MT4701945) |
| | | | | | | CC12.1 | 3504 | QQLNSYPP | Thomas Rogers et al., |
| | | | | | | | | KFT | 2020 |
| | | | | | | | | | (https://science.sciencemag. |
| | | | | | | | | | org/content/early/ |
| | | | | | | | | | 2020/06/15/science.abc7520); |
| | | | | | | | | | Meng Yuan et al., 2020 |
| | | | | | | | | | (https://www.biorxiv. |
| | | | | | | | | | org/content/10.1101/ |
| | | | | | | | | | 2020.06.08.141267v1) |
| | | | | | | CC12.10 | 3505 | SSYAGSST | Thomas Rogers et al., |
| | | | | | | | | QV | 2020 |
| | | | | | | | | | (https://science.sciencemag. |
| | | | | | | | | | org/content/early/ |
| | | | | | | | | | 2020/06/15/science.abc7520) |
| | | | | | | CC12.11 | 3506 | SSYTSSSAQ | Thomas Rogers et al., |
| | | | | | | | | L | 2020 |
| | | | | | | | | | (https://science.sciencemag. |
| | | | | | | | | | org/content/early/ |
| | | | | | | | | | 2020/06/15/science.abc7520) |
| | | | | | | CC12.12 | 3507 | SSYTSGST | Thomas Rogers et al., |
| | | | | | | | | QV | 2020 |
| | | | | | | | | | (https://science.sciencemag. |
| | | | | | | | | | org/content/early/ |
| | | | | | | | | | 2020/06/15/science.abc7520) |
| | | | | | | CC12.13 | 3508 | QQYDNLPI | Thomas Rogers et al., |
| | | | | | | | | T | 2020 |
| | | | | | | | | | (https://science.sciencemag. |
| | | | | | | | | | org/content/early/ |
| | | | | | | | | | 2020/06/15/science.abc7520) |
| | | | | | | CC12.14 | 3509 | MQGTHW | Thomas Rogers et al., |
| | | | | | | | | PPT | 2020 |
| | | | | | | | | | (https://science.sciencemag. |
| | | | | | | | | | org/content/early/ |
| | | | | | | | | | 2020/06/15/science.abc7520) |
| | | | | | | CC12.15 | 3510 | QSYDSSLS | Thomas Rogers et al., |
| | | | | | | | | GW | 2020 |
| | | | | | | | | | (https://science.sciencemag. |
| | | | | | | | | | org/content/early/ |
| | | | | | | | | | 2020/06/15/science.abc7520) |
| | | | | | | CC12.16 | 3511 | QVWDSSS | Thomas Rogers et al., |
| | | | | | | | | DPWV | 2020 |
| | | | | | | | | | (https://science.sciencemag. |
| | | | | | | | | | org/content/early/ |
| | | | | | | | | | 2020/06/15/science.abc7520) |
| | | | | | | CC12.17 | 3512 | QVWDSSS | Thomas Rogers et al., |
| | | | | | | | | DHPW | 2020 |
| | | | | | | | | | (https://science.sciencemag. |
| | | | | | | | | | org/content/early/ |
| | | | | | | | | | 2020/06/15/science.abc7520) |
| | | | | | | CC12.18 | 3513 | QSYDSSNH | Thomas Rogers et al., |
| | | | | | | | | EEIWV | 2020 |
| | | | | | | | | | (https://science.sciencemag. |
| | | | | | | | | | org/content/early/ |
| | | | | | | | | | 2020/06/15/science.abc7520) |
| | | | | | | CC12.19 | 3514 | QVWDNNS | Thomas Rogers et al., |
| | | | | | | | | DHLV | 2020 |
| | | | | | | | | | (https://science.sciencemag. |
| | | | | | | | | | org/content/early/ |
| | | | | | | | | | 2020/06/15/science.abc7520) |
| | | | | | | CC12.2 | 3515 | QQYGSSPR | Thomas Rogers et al., |
| | | | | | | | | T | 2020 |
| | | | | | | | | | (https://science.sciencemag. |
| | | | | | | | | | org/content/early/ |
| | | | | | | | | | 2020/06/15/science.abc7520) |
| | | | | | | CC12.20 | 3516 | AAWDDSL | Thomas Rogers et al., |
| | | | | | | | | SGRW | 2020 |
| | | | | | | | | | (https://science.sciencemag. |
| | | | | | | | | | org/content/early/ |
| | | | | | | | | | 2020/06/15/science.abc7520) |
| | | | | | | CC12.21 | 3517 | AAWDDSL | Thomas Rogers et al., |
| | | | | | | | | NGPV | 2020 |
| | | | | | | | | | (https://science.sciencemag. |
| | | | | | | | | | org/content/early/ |
| | | | | | | | | | 2020/06/15/science.abc7520) |
| | | | | | | CC12.23 | 3518 | QSADSSGT | Thomas Rogers et al., |
| | | | | | | | | YLVV | 2020 |
| | | | | | | | | | (https://science.sciencemag. |
| | | | | | | | | | org/content/early/ |
| | | | | | | | | | 2020/06/15/science.abc7520) |
| | | | | | | CC12.24 | 3519 | QQSYSTP | Thomas Rogers et al., |
| | | | | | | | | WT | 2020 |
| | | | | | | | | | (https://science.sciencemag. |
| | | | | | | | | | org/content/early/ |
| | | | | | | | | | 2020/06/15/science.abc7520) |
| | | | | | | CC12.25 | 3520 | AAWDDSL | Thomas Rogers et al., |
| | | | | | | | | NGPV | 2020 |
| | | | | | | | | | (https://science.sciencemag. |
| | | | | | | | | | org/content/early/ |
| | | | | | | | | | 2020/06/15/science.abc7520) |
| | | | | | | CC12.26 | 3521 | LLYYGGAQ | Thomas Rogers et al., |
| | | | | | | | | RWV | 2020 |
| | | | | | | | | | (https://science.sciencemag. |
| | | | | | | | | | org/content/early/ |
| | | | | | | | | | 2020/06/15/science.abc7520) |
| | | | | | | CC12.27 | 3522 | YSYAGSSTF | Thomas Rogers et al., |
| | | | | | | | | V | 2020 |
| | | | | | | | | | (https://science.sciencemag. |
| | | | | | | | | | org/content/early/ |
| | | | | | | | | | 2020/06/15/science.abc7520) |
| | | | | | | CC12.28 | 3523 | AAWDDSL | Thomas Rogers et al., |
| | | | | | | | | SGWV | 2020 |
| | | | | | | | | | (https://science.sciencemag. |
| | | | | | | | | | org/content/early/ |
| | | | | | | | | | 2020/06/15/science.abc7520) |
| | | | | | | CC12.3 | 3524 | QQYGSSPR | Thomas Rogers et al., |
| | | | | | | | | T | 2020 |
| | | | | | | | | | (https://science.sciencemag. |
| | | | | | | | | | org/content/early/ |
| | | | | | | | | | 2020/06/15/science.abc7520); |
| | | | | | | | | | Meng Yuan et al., 2020 |
| | | | | | | | | | (https://www.biorxiv. |
| | | | | | | | | | org/content/10.1101/ |
| | | | | | | | | | 2020.06.08.141267v1) |
| | | | | | | CC12.4 | 3525 | TSYAGSNN | Thomas Rogers et al., |
| | | | | | | | | FV | 2020 |
| | | | | | | | | | (https://science.sciencemag. |
| | | | | | | | | | org/content/early/ |
| | | | | | | | | | 2020/06/15/science.abc7520) |
| | | | | | | CC12.5 | 3526 | SSYTSSSTQ | Thomas Rogers et al., |
| | | | | | | | | V | 2020 |
| | | | | | | | | | (https://science.sciencemag. |
| | | | | | | | | | org/content/early/ |
| | | | | | | | | | 2020/06/15/science.abc7520) |
| | | | | | | CC12.6 | 3527 | SSYTSSSTQ | Thomas Rogers et al., |
| | | | | | | | | V | 2020 |
| | | | | | | | | | (https://science.sciencemag. |
| | | | | | | | | | org/content/early/ |
| | | | | | | | | | 2020/06/15/science.abc7520) |
| | | | | | | CC12.7 | 3528 | SSYTVSSTQ | Thomas Rogers et al., |
| | | | | | | | | V | 2020 |
| | | | | | | | | | (https://science.sciencemag. |
| | | | | | | | | | org/content/early/ |
| | | | | | | | | | 2020/06/15/science.abc7520) |
| | | | | | | CC12.8 | 3529 | SSYTSSSAQ | Thomas Rogers et al., |
| | | | | | | | | L | 2020 |
| | | | | | | | | | (https://science.sciencemag. |
| | | | | | | | | | org/content/early/ |
| | | | | | | | | | 2020/06/15/science.abc7520) |
| | | | | | | CC12.9 | 3530 | SSYTSSSAQ | Thomas Rogers et al., |
| | | | | | | | | V | 2020 |
| | | | | | | | | | (https://science.sciencemag. |
| | | | | | | | | | org/content/early/ |
| | | | | | | | | | 2020/06/15/science.abc7520) |
| | | | | | | CC6.29 | 3531 | QQSYSTPP | Thomas Rogers et al., |
| | | | | | | | | T | 2020 |
| | | | | | | | | | (https://science.sciencemag. |
| | | | | | | | | | org/content/early/ |
| | | | | | | | | | 2020/06/15/science.abc7520) |
| | | | | | | CC6.30 | 3532 | QQSYSTPR | Thomas Rogers et al., |
| | | | | | | | | T | 2020 |
| | | | | | | | | | (https://science.sciencemag. |
| | | | | | | | | | org/content/early/ |
| | | | | | | | | | 2020/06/15/science.abc7520) |
| | | | | | | CC6.31 | 3533 | LQHNSYPIL | Thomas Rogers et al., |
| | | | | | | | | T | 2020 |
| | | | | | | | | | (https://science.sciencemag. |
| | | | | | | | | | org/content/early/ |
| | | | | | | | | | 2020/06/15/science.abc7520) |
| | | | | | | CC6.32 | 3534 | QVWDSSS | Thomas Rogers et al., |
| | | | | | | | | DHPYV | 2020 |
| | | | | | | | | | (https://science.sciencemag. |
| | | | | | | | | | org/content/early/ |
| | | | | | | | | | 2020/06/15/science.abc7520) |
| | | | | | | CC6.33 | 3535 | QHYGSSL | Thomas Rogers et al., |
| | | | | | | | | WT | 2020 |
| | | | | | | | | | (https://science.sciencemag. |
| | | | | | | | | | org/content/early/ |
| | | | | | | | | | 2020/06/15/science.abc7520) |
| | | | | | | Clone11- | 3536 | FQGSHVPL | Nadezhda Antipova et |
| | | | | | | 9 | | T | al., 2020 |
| | | | | | | CnC2t1p1_ | 3537 | QQRSNWP | Christoph Kreer et al., |
| | | | | | | B10 | | PALT | 2020 |
| | | | | | | | | | (https://doi.org/10.1016/ |
| | | | | | | | | | j.cell.2020.06.044) |
| | | | | | | CnC2t1p1_ | 3538 | CSYAGSST | Christoph Kreer et al., |
| | | | | | | B4 | | WV | 2020 |
| | | | | | | | | | (https://doi.org/10.1016/ |
| | | | | | | | | | j.cell.2020.06.044) |
| | | | | | | CnC2t1p1_ | 3539 | MQALQTP | Christoph Kreer et al., |
| | | | | | | D6 | | GT | 2020 |
| | | | | | | | | | (https://doi.org/10.1016/ |
| | | | | | | | | | j.cell.2020.06.044) |
| | | | | | | CnC2t1p1_ | 3540 | MQALQTP | Christoph Kreer et al., |
| | | | | | | E12 | | GT | 2020 |
| | | | | | | | | | (https://doi.org/10.1016/ |
| | | | | | | | | | j.cell.2020.06.044) |
| | | | | | | CnC2t1p1_ | 3541 | CSYAGVRT | Christoph Kreer et al., |
| | | | | | | E8 | | W | 2020 |
| | | | | | | | | | (https://doi.org/10.1016/ |
| | | | | | | | | | j.cell.2020.06.044) |
| | | | | | | CnC2t1p1_ | 3542 | CSYAGVRT | Christoph Kreer et al., |
| | | | | | | G6 | | W | 2020 |
| | | | | | | | | | (https://doi.org/10.1016/ |
| | | | | | | | | | j.cell.2020.06.044) |
| | | | | | | COV2- | 3543 | QQYGSSP | Seth Zost et al., 2020 |
| | | | | | | 2006 | | WT | (https://www.nature.com/ |
| | | | | | | | | | articles/s41591-020- |
| | | | | | | | | | 0998-x) |
| | | | | | | COV2- | 3544 | QQYDNLLF | Seth Zost et al., 2020 |
| | | | | | | 2007 | | T | (https://www.nature.com/ |
| | | | | | | | | | articles/s41591-020- |
| | | | | | | | | | 0998-x) |
| | | | | | | COV2- | 3545 | SSYTSSSTL | Seth Zost et al., 2020 |
| | | | | | | 2009 | | WV | (https://www.nature.com/ |
| | | | | | | | | | articles/s41591-020- |
| | | | | | | | | | 0998-x) |
| | | | | | | COV2- | 3546 | QQSYSTFT | Seth Zost et al., 2020 |
| | | | | | | 2011 | | | (https://www.nature.com/ |
| | | | | | | | | | articles/s41591-020- |
| | | | | | | | | | 0998-x) |
| | | | | | | COV2- | 3547 | QQYYTAPL | Seth Zost et al., 2020 |
| | | | | | | 2013 | | T | (https://www.nature.com/ |
| | | | | | | | | | articles/s41591-020- |
| | | | | | | | | | 0998-x) |
| | | | | | | COV2- | 3548 | QQRSNWP | Seth Zost et al., 2020 |
| | | | | | | 2015 | | PYT | (https://www.nature.com/ |
| | | | | | | | | | articles/s41591-020- |
| | | | | | | | | | 0998-x) |
| | | | | | | COV2- | 3549 | SSYTSSTT | Seth Zost et al., 2020 |
| | | | | | | 2016 | | WV | (https://www.nature.com/ |
| | | | | | | | | | articles/s41591-020- |
| | | | | | | | | | 0998-x) |
| | | | | | | COV2- | 3550 | QSFDSSLS | Seth Zost et al., 2020 |
| | | | | | | 2017 | | GSDV | (https://www.nature.com/ |
| | | | | | | | | | articles/s41591-020- |
| | | | | | | | | | 0998-x) |
| | | | | | | COV2- | 3551 | AAWDASL | Seth Zost et al., 2020 |
| | | | | | | 2021 | | SGHVV | (https://www.nature.com/ |
| | | | | | | | | | articles/s41591-020- |
| | | | | | | | | | 0998-x) |
| | | | | | | COV2- | 3552 | MQRIEFP | Seth Zost et al., 2020 |
| | | | | | | 2022 | | WT | (https://www.nature.com/ |
| | | | | | | | | | articles/s41591-020- |
| | | | | | | | | | 0998-x) |
| | | | | | | COV2- | 3553 | QQYYTAPL | Seth Zost et al., 2020 |
| | | | | | | 2025 | | T | (https://www.nature.com/ |
| | | | | | | | | | articles/s41591-020- |
| | | | | | | | | | 0998-x) |
| | | | | | | COV2- | 3554 | QQYNSYP | Seth Zost et al., 2020 |
| | | | | | | 2026 | | WT | (https://www.nature.com/ |
| | | | | | | | | | articles/s41591-020- |
| | | | | | | | | | 0998-x) |
| | | | | | | COV2- | 3555 | QSADSSGT | Seth Zost et al., 2020 |
| | | | | | | 2027 | | YFWV | (https://www.nature.com/ |
| | | | | | | | | | articles/s41591-020- |
| | | | | | | | | | 0998-x) |
| | | | | | | COV2- | 3556 | QQYYTAPL | Seth Zost et al., 2020 |
| | | | | | | 2028 | | T | (https://www.nature.com/ |
| | | | | | | | | | articles/s41591-020- |
| | | | | | | | | | 0998-x) |
| | | | | | | COV2- | 3557 | SVWDDSL | Seth Zost et al., 2020 |
| | | | | | | 2029 | | NGPL | (https://www.nature.com/ |
| | | | | | | | | | articles/s41591-020- |
| | | | | | | | | | 0998-x) |
| | | | | | | COV2- | 3558 | QSSDSSGV | Seth Zost et al., 2020 |
| | | | | | | 2031 | | V | (https://www.nature.com/ |
| | | | | | | | | | articles/s41591-020- |
| | | | | | | | | | 0998-x) |
| | | | | | | COV2- | 3559 | QQYNSYS | Seth Zost et al., 2020 |
| | | | | | | 2032 | | WT | (https://www.nature.com/ |
| | | | | | | | | | articles/s41591-020- |
| | | | | | | | | | 0998-x) |
| | | | | | | COV2- | 3560 | SVWDDSL | Seth Zost et al., 2020 |
| | | | | | | 2033 | | NGPL | (https://www.nature.com/ |
| | | | | | | | | | articles/s41591-020- |
| | | | | | | | | | 0998-x) |
| | | | | | | COV2- | 3561 | QQSYSTPP | Seth Zost et al., 2020 |
| | | | | | | 2034 | | T | (https://www.nature.com/ |
| | | | | | | | | | articles/s41591-020- |
| | | | | | | | | | 0998-x) |
| | | | | | | COV2- | 3562 | QQSYNTPR | Seth Zost et al., 2020 |
| | | | | | | 2035 | | T | (https://www.nature.com/ |
| | | | | | | | | | articles/s41591-020- |
| | | | | | | | | | 0998-x) |
| | | | | | | COV2- | 3563 | QQTNSFPT | Seth Zost et al., 2020 |
| | | | | | | 2037 | | | (https://www.nature.com/ |
| | | | | | | | | | articles/s41591-020- |
| | | | | | | | | | 0998-x) |
| | | | | | | COV2- | 3564 | QQYGNSP | Seth Zost et al., 2020 |
| | | | | | | 2039 | | Q | (https://www.nature.com/ |
| | | | | | | | | | articles/s41591-020- |
| | | | | | | | | | 0998-x) |
| | | | | | | COV2- | 3565 | QQYNSYST | Seth Zost et al., 2020 |
| | | | | | | 2041 | | WT | (https://www.nature.com/ |
| | | | | | | | | | articles/s41591-020- |
| | | | | | | | | | 0998-x) |
| | | | | | | COV2- | 3566 | QQSYNTPY | Seth Zost et al., 2020 |
| | | | | | | 2046 | | T | (https://www.nature.com/ |
| | | | | | | | | | articles/s41591-020- |
| | | | | | | | | | 0998-x) |
| | | | | | | COV2- | 3567 | AAWDDSL | Seth Zost et al., 2020 |
| | | | | | | 2050 | | NALV | (https://www.nature.com/ |
| | | | | | | | | | articles/s41591-020- |
| | | | | | | | | | 0998-x) |
| | | | | | | COV2- | 3568 | QQSYSTPG | Seth Zost et al., 2020 |
| | | | | | | 2051 | | T | (https://www.nature.com/ |
| | | | | | | | | | articles/s41591-020- |
| | | | | | | | | | 0998-x) |
| | | | | | | COV2- | 3569 | QQSDNLP | Seth Zost et al., 2020 |
| | | | | | | 2054 | | MYT | (https://www.nature.com/ |
| | | | | | | | | | articles/s41591-020- |
| | | | | | | | | | 0998-x) |
| | | | | | | COV2- | 3570 | QVWDSSS | Seth Zost et al., 2020 |
| | | | | | | 2055 | | DHHW | (https://www.nature.com/ |
| | | | | | | | | | articles/s41591-020- |
| | | | | | | | | | 0998-x) |
| | | | | | | COV2- | 3571 | LVWDDSL | Seth Zost et al., 2020 |
| | | | | | | 2064 | | NGLV | (https://www.nature.com/ |
| | | | | | | | | | articles/s41591-020- |
| | | | | | | | | | 0998-x) |
| | | | | | | COV2- | 3572 | QSYDSRLS | Seth Zost et al., 2020 |
| | | | | | | 2068 | | GFW | (https://www.nature.com/ |
| | | | | | | | | | articles/s41591-020- |
| | | | | | | | | | 0998-x) |
| | | | | | | COV2- | 3573 | QQSYTTLL | Seth Zost et al., 2020 |
| | | | | | | 2070 | | T | (https://www.nature.com/ |
| | | | | | | | | | articles/s41591-020- |
| | | | | | | | | | 0998-x) |
| | | | | | | COV2- | 3574 | QQYGSSP | Seth Zost et al., 2020 |
| | | | | | | 2072 | | WT | (https://www.nature.com/ |
| | | | | | | | | | articles/s41591-020- |
| | | | | | | | | | 0998-x) |
| | | | | | | COV2- | 3575 | QSYDSSLS | Seth Zost et al., 2020 |
| | | | | | | 2078 | | DSV | (https://www.nature.com/ |
| | | | | | | | | | articles/s41591-020- |
| | | | | | | | | | 0998-x) |
| | | | | | | COV2- | 3576 | QLLSSHPL | Seth Zost et al., 2020 |
| | | | | | | 2080 | | T | (https://www.nature.com/ |
| | | | | | | | | | articles/s41591-020- |
| | | | | | | | | | 0998-x) |
| | | | | | | COV2- | 3577 | SSRDSSGS | Seth Zost et al., 2020 |
| | | | | | | 2082 | | AW | (https://www.nature.com/ |
| | | | | | | | | | articles/s41591-020- |
| | | | | | | | | | 0998-x) |
| | | | | | | COV2- | 3578 | QQYASLPF | Seth Zost et al., 2020 |
| | | | | | | 2083 | | T | (https://www.nature.com/ |
| | | | | | | | | | articles/s41591-020- |
| | | | | | | | | | 0998-x) |
| | | | | | | COV2- | 3579 | SSRDSSGS | Seth Zost et al., 2020 |
| | | | | | | 2094 | | AW | (https://www.nature.com/ |
| | | | | | | | | | articles/s41591-020- |
| | | | | | | | | | 0998-x) |
| | | | | | | COV2- | 3580 | AVWDDSL | Seth Zost et al., 2020 |
| | | | | | | 2096 | | SGLV | (https://www.nature.com/ |
| | | | | | | | | | articles/s41591-020- |
| | | | | | | | | | 0998-x) |
| | | | | | | COV2- | 3581 | QQSYSTP | Seth Zost et al., 2020 |
| | | | | | | 2097 | | WT | (https://www.nature.com/ |
| | | | | | | | | | articles/s41591-020- |
| | | | | | | | | | 0998-x) |
| | | | | | | COV2- | 3582 | HQYSSWP | Seth Zost et al., 2020 |
| | | | | | | 2098 | | QT | (https://www.nature.com/ |
| | | | | | | | | | articles/s41591-020- |
| | | | | | | | | | 0998-x) |
| | | | | | | COV2- | 3583 | QSYDGISR | Seth Zost et al., 2020 |
| | | | | | | 2103 | | AWV | (https://www.nature.com/ |
| | | | | | | | | | articles/s41591-020- |
| | | | | | | | | | 0998-x) |
| | | | | | | COV2- | 3584 | MQALQTP | Seth Zost et al., 2020 |
| | | | | | | 2108 | | LT | (https://www.nature.com/ |
| | | | | | | | | | articles/s41591-020- |
| | | | | | | | | | 0998-x) |
| | | | | | | COV2- | 3585 | QQSYSSLSI | Seth Zost et al., 2020 |
| | | | | | | 2110 | | T | (https://www.nature.com/ |
| | | | | | | | | | articles/s41591-020- |
| | | | | | | | | | 0998-x) |
| | | | | | | COV2- | 3586 | QQSYEIPP | Seth Zost et al., 2020 |
| | | | | | | 2111 | | WT | (https://www.nature.com/ |
| | | | | | | | | | articles/s41591-020- |
| | | | | | | | | | 0998-x) |
| | | | | | | COV2- | 3587 | QQYDSLP | Seth Zost et al., 2020 |
| | | | | | | 2113 | | PV | (https://www.nature.com/ |
| | | | | | | | | | articles/s41591-020- |
| | | | | | | | | | 0998-x) |
| | | | | | | COV2- | 3588 | QSYDSSLS | Seth Zost et al., 2020 |
| | | | | | | 2114 | | GSV | (https://www.nature.com/ |
| | | | | | | | | | articles/s41591-020- |
| | | | | | | | | | 0998-x) |
| | | | | | | COV2- | 3589 | QSYDSGNP | Seth Zost et al., 2020 |
| | | | | | | 2128 | | | (https://www.nature.com/ |
| | | | | | | | | | articles/s41591-020- |
| | | | | | | | | | 0998-x) |
| | | | | | | COV2- | 3590 | QQYYSTLT | Seth Zost et al., 2020 |
| | | | | | | 2130 | | | (https://www.nature.com/ |
| | | | | | | | | | articles/s41591-020- |
| | | | | | | | | | 0998-x) |
| | | | | | | COV2- | 3591 | QQYDNLP | Seth Zost et al., 2020 |
| | | | | | | 2132 | | PV | (https://www.nature.com/ |
| | | | | | | | | | articles/s41591-020- |
| | | | | | | | | | 0998-x) |
| | | | | | | COV2- | 3592 | QSYDGINR | Seth Zost et al., 2020 |
| | | | | | | 2137 | | WLV | (https://www.nature.com/ |
| | | | | | | | | | articles/s41591-020- |
| | | | | | | | | | 0998-x) |
| | | | | | | COV2- | 3593 | GADHGSG | Seth Zost et al., 2020 |
| | | | | | | 2142 | | SNFEYVV | (https://www.nature.com/ |
| | | | | | | | | | articles/s41591-020- |
| | | | | | | | | | 0998-x) |
| | | | | | | COV2- | 3594 | AAWDDSL | Seth Zost et al., 2020 |
| | | | | | | 2143 | | NGYV | (https://www.nature.com/ |
| | | | | | | | | | articles/s41591-020- |
| | | | | | | | | | 0998-x) |
| | | | | | | COV2- | 3595 | QQLNSYPV | Seth Zost et al., 2020 |
| | | | | | | 2146 | | T | (https://www.nature.com/ |
| | | | | | | | | | articles/s41591-020- |
| | | | | | | | | | 0998-x) |
| | | | | | | COV2- | 3596 | SSYTSSSTL | Seth Zost et al., 2020 |
| | | | | | | 2147 | | LYV | (https://www.nature.com/ |
| | | | | | | | | | articles/s41591-020- |
| | | | | | | | | | 0998-x) |
| | | | | | | COV2- | 3597 | QVWDSSS | Seth Zost et al., 2020 |
| | | | | | | 2150 | | DHPGV | (https://www.nature.com/ |
| | | | | | | | | | articles/s41591-020- |
| | | | | | | | | | 0998-x) |
| | | | | | | COV2- | 3598 | HYRSNWP | Seth Zost et al., 2020 |
| | | | | | | 2151 | | PVLT | (https://www.nature.com/ |
| | | | | | | | | | articles/s41591-020- |
| | | | | | | | | | 0998-x) |
| | | | | | | COV2- | 3599 | QQYDNLYS | Seth Zost et al., 2020 |
| | | | | | | 2158 | | VH | (https://www.nature.com/ |
| | | | | | | | | | articles/s41591-020- |
| | | | | | | | | | 0998-x) |
| | | | | | | COV2- | 3600 | SSYTSSSTL | Seth Zost et al., 2020 |
| | | | | | | 2159 | | LYV | (https://www.nature.com/ |
| | | | | | | | | | articles/s41591-020- |
| | | | | | | | | | 0998-x) |
| | | | | | | COV2- | 3601 | SSYTSSSTL | Seth Zost et al., 2020 |
| | | | | | | 2160 | | LYV | (https://www.nature.com/ |
| | | | | | | | | | articles/s41591-020- |
| | | | | | | | | | 0998-x) |
| | | | | | | COV2- | 3602 | QLLNSHPL | Seth Zost et al., 2020 |
| | | | | | | 2165 | | T | (https://www.nature.com/ |
| | | | | | | | | | articles/s41591-020- |
| | | | | | | | | | 0998-x) |
| | | | | | | COV2- | 3603 | QHRTNWP | Seth Zost et al., 2020 |
| | | | | | | 2166 | | PLFT | (https://www.nature.com/ |
| | | | | | | | | | articles/s41591-020- |
| | | | | | | | | | 0998-x) |
| | | | | | | COV2- | 3604 | QAWDSST | Seth Zost et al., 2020 |
| | | | | | | 2171 | | GV | (https://www.nature.com/ |
| | | | | | | | | | articles/s41591-020- |
| | | | | | | | | | 0998-x) |
| | | | | | | COV2- | 3605 | QQYNTYSG | Seth Zost et al., 2020 |
| | | | | | | 2173 | | T | (https://www.nature.com/ |
| | | | | | | | | | articles/s41591-020- |
| | | | | | | | | | 0998-x) |
| | | | | | | COV2- | 3606 | QAWDSST | Seth Zost et al., 2020 |
| | | | | | | 2175 | | GV | (https://www.nature.com/ |
| | | | | | | | | | articles/s41591-020- |
| | | | | | | | | | 0998-x) |
| | | | | | | COV2- | 3607 | QQTYSTF | Seth Zost et al., 2020 |
| | | | | | | 2177 | | WT | (https://www.nature.com/ |
| | | | | | | | | | articles/s41591-020- |
| | | | | | | | | | 0998-x) |
| | | | | | | COV2- | 3608 | QAWDSST | Seth Zost et al., 2020 |
| | | | | | | 2178 | | AV | (https://www.nature.com/ |
| | | | | | | | | | articles/s41591-020- |
| | | | | | | | | | 0998-x) |
| | | | | | | COV2- | 3609 | SSYTSSRAV | Seth Zost et al., 2020 |
| | | | | | | 2183 | | L | (https://www.nature.com/ |
| | | | | | | | | | articles/s41591-020- |
| | | | | | | | | | 0998-x) |
| | | | | | | COV2- | 3610 | QVWDSSS | Seth Zost et al., 2020 |
| | | | | | | 2187 | | DHPEWV | (https://www.nature.com/ |
| | | | | | | | | | articles/s41591-020- |
| | | | | | | | | | 0998-x) |
| | | | | | | COV2- | 3611 | QQSYSTPT | Seth Zost et al., 2020 |
| | | | | | | 2189 | | | (https://www.nature.com/ |
| | | | | | | | | | articles/s41591-020- |
| | | | | | | | | | 0998-x) |
| | | | | | | COV2- | 3612 | SSYTSSSTH | Seth Zost et al., 2020 |
| | | | | | | 2190 | | W | (https://www.nature.com/ |
| | | | | | | | | | articles/s41591-020- |
| | | | | | | | | | 0998-x) |
| | | | | | | COV2- | 3613 | QQYNTYSG | Seth Zost et al., 2020 |
| | | | | | | 2191 | | T | (https://www.nature.com/ |
| | | | | | | | | | articles/s41591-020- |
| | | | | | | | | | 0998-x) |
| | | | | | | COV2- | 3614 | QQYYTAPL | Seth Zost et al., 2020 |
| | | | | | | 2195 | | T | (https://www.nature.com/ |
| | | | | | | | | | articles/s41591-020- |
| | | | | | | | | | 0998-x) |
| | | | | | | COV2- | 3615 | QHYGSSRG | Seth Zost et al., 2020 |
| | | | | | | 2196 | | WT | (https://www.nature.com/ |
| | | | | | | | | | articles/s41591-020- |
| | | | | | | | | | 0998-x) |
| | | | | | | COV2- | 3616 | QQYGRSPI | Seth Zost et al., 2020 |
| | | | | | | 2197 | | T | (https://www.nature.com/ |
| | | | | | | | | | articles/s41591-020- |
| | | | | | | | | | 0998-x) |
| | | | | | | COV2- | 3617 | QAWDSST | Seth Zost et al., 2020 |
| | | | | | | 2199 | | W | (https://www.nature.com/ |
| | | | | | | | | | articles/s41591-020- |
| | | | | | | | | | 0998-x) |
| | | | | | | COV2- | 3618 | QQSYSTPY | Seth Zost et al., 2020 |
| | | | | | | 2203 | | T | (https://www.nature.com/ |
| | | | | | | | | | articles/s41591-020- |
| | | | | | | | | | 0998-x) |
| | | | | | | COV2- | 3619 | QSYDSSLG | Seth Zost et al., 2020 |
| | | | | | | 2207 | | AL | (https://www.nature.com/ |
| | | | | | | | | | articles/s41591-020- |
| | | | | | | | | | 0998-x) |
| | | | | | | COV2- | 3620 | QQANSFPP | Seth Zost et al., 2020 |
| | | | | | | 2210 | | T | (https://www.nature.com/ |
| | | | | | | | | | articles/s41591-020- |
| | | | | | | | | | 0998-x) |
| | | | | | | COV2- | 3621 | QQRGNW | Seth Zost et al., 2020 |
| | | | | | | 2212 | | WT | (https://www.nature.com/ |
| | | | | | | | | | articles/s41591-020- |
| | | | | | | | | | 0998-x) |
| | | | | | | COV2- | 3622 | QHRTNWP | Seth Zost et al., 2020 |
| | | | | | | 2214 | | PLFT | (https://www.nature.com/ |
| | | | | | | | | | articles/s41591-020- |
| | | | | | | | | | 0998-x) |
| | | | | | | COV2- | 3623 | QQCYNWP | Seth Zost et al., 2020 |
| | | | | | | 2215 | | PWT | (https://www.nature.com/ |
| | | | | | | | | | articles/s41591-020- |
| | | | | | | | | | 0998-x) |
| | | | | | | COV2- | 3624 | HKRSNWP | Seth Zost et al., 2020 |
| | | | | | | 2216 | | PSLT | (https://www.nature.com/ |
| | | | | | | | | | articles/s41591-020- |
| | | | | | | | | | 0998-x) |
| | | | | | | COV2- | 3625 | QQYYSISW | Seth Zost et al., 2020 |
| | | | | | | 2218 | | T | (https://www.nature.com/ |
| | | | | | | | | | articles/s41591-020- |
| | | | | | | | | | 0998-x) |
| | | | | | | COV2- | 3626 | CSYAVSTTY | Seth Zost et al., 2020 |
| | | | | | | 2222 | | VI | (https://www.nature.com/ |
| | | | | | | | | | articles/s41591-020- |
| | | | | | | | | | 0998-x) |
| | | | | | | COV2- | 3627 | QQYYSTP | Seth Zost et al., 2020 |
| | | | | | | 2224 | | WT | (https://www.nature.com/ |
| | | | | | | | | | articles/s41591-020- |
| | | | | | | | | | 0998-x) |
| | | | | | | COV2- | 3628 | GADHGSG | Seth Zost et al., 2020 |
| | | | | | | 2226 | | SNFVFVV | (https://www.nature.com/ |
| | | | | | | | | | articles/s41591-020- |
| | | | | | | | | | 0998-x) |
| | | | | | | COV2- | 3629 | MQALQTP | Seth Zost et al., 2020 |
| | | | | | | 2227 | | | (https://www.nature.com/ |
| | | | | | | | | | articles/s41591-020- |
| | | | | | | | | | 0998-x) |
| | | | | | | COV2- | 3630 | QQYNYWP | Seth Zost et al., 2020 |
| | | | | | | 2228 | | PLT | (https://www.nature.com/ |
| | | | | | | | | | articles/s41591-020- |
| | | | | | | | | | 0998-x) |
| | | | | | | COV2- | 3631 | QVWDSSS | Seth Zost et al., 2020 |
| | | | | | | 2231 | | DHHW | (https://www.nature.com/ |
| | | | | | | | | | articles/s41591-020- |
| | | | | | | | | | 0998-x) |
| | | | | | | COV2- | 3632 | QQYNTYS | Seth Zost et al., 2020 |
| | | | | | | 2235 | | QT | (https://www.nature.com/ |
| | | | | | | | | | articles/s41591-020- |
| | | | | | | | | | 0998-x) |
| | | | | | | COV2- | 3633 | QAWDND | Seth Zost et al., 2020 |
| | | | | | | 2238 | | AGW | (https://www.nature.com/ |
| | | | | | | | | | articles/s41591-020- |
| | | | | | | | | | 0998-x) |
| | | | | | | COV2- | 3634 | QQYNSHP | Seth Zost et al., 2020 |
| | | | | | | 2239 | | PT | (https://www.nature.com/ |
| | | | | | | | | | articles/s41591-020- |
| | | | | | | | | | 0998-x) |
| | | | | | | COV2- | 3635 | SAYAGSNN | Seth Zost et al., 2020 |
| | | | | | | 2240 | | LV | (https://www.nature.com/ |
| | | | | | | | | | articles/s41591-020- |
| | | | | | | | | | 0998-x) |
| | | | | | | COV2- | 3636 | GADHGSG | Seth Zost et al., 2020 |
| | | | | | | 2241 | | SNFEYVV | (https://www.nature.com/ |
| | | | | | | | | | articles/s41591-020- |
| | | | | | | | | | 0998-x) |
| | | | | | | COV2- | 3637 | CSYAGIVL | Seth Zost et al., 2020 |
| | | | | | | 2243 | | | (https://www.nature.com/ |
| | | | | | | | | | articles/s41591-020- |
| | | | | | | | | | 0998-x) |
| | | | | | | COV2- | 3638 | QSGDSSGT | Seth Zost et al., 2020 |
| | | | | | | 2245 | | YVV | (https://www.nature.com/ |
| | | | | | | | | | articles/s41591-020- |
| | | | | | | | | | 0998-x) |
| | | | | | | COV2- | 3639 | QSVDRSGT | Seth Zost et al., 2020 |
| | | | | | | 2248 | | YFNWV | (https://www.nature.com/ |
| | | | | | | | | | articles/s41591-020- |
| | | | | | | | | | 0998-x) |
| | | | | | | COV2- | 3640 | QQSYSTPY | Seth Zost et al., 2020 |
| | | | | | | 2250 | | T | (https://www.nature.com/ |
| | | | | | | | | | articles/s41591-020- |
| | | | | | | | | | 0998-x) |
| | | | | | | COV2- | 3641 | LLYYGGA | Seth Zost et al., 2020 |
| | | | | | | 2251 | | WV | (https://www.nature.com/ |
| | | | | | | | | | articles/s41591-020- |
| | | | | | | | | | 0998-x) |
| | | | | | | COV2- | 3642 | QSYDGSN | Seth Zost et al., 2020 |
| | | | | | | 2253 | | HAW | (https://www.nature.com/ |
| | | | | | | | | | articles/s41591-020- |
| | | | | | | | | | 0998-x) |
| | | | | | | COV2- | 3643 | QVWDSSS | Seth Zost et al., 2020 |
| | | | | | | 2256 | | DPW | (https://www.nature.com/ |
| | | | | | | | | | articles/s41591-020- |
| | | | | | | | | | 0998-x) |
| | | | | | | COV2- | 3644 | MQALQTP | Seth Zost et al., 2020 |
| | | | | | | 2257 | | QT | (https://www.nature.com/ |
| | | | | | | | | | articles/s41591-020- |
| | | | | | | | | | 0998-x) |
| | | | | | | COV2- | 3645 | QQYGGSLT | Seth Zost et al., 2020 |
| | | | | | | 2258 | | | (https://www.nature.com/ |
| | | | | | | | | | articles/s41591-020- |
| | | | | | | | | | 0998-x) |
| | | | | | | COV2- | 3646 | QAWDSSL | Seth Zost et al., 2020 |
| | | | | | | 2260 | | W | (https://www.nature.com/ |
| | | | | | | | | | articles/s41591-020- |
| | | | | | | | | | 0998-x) |
| | | | | | | COV2- | 3647 | SSYTSSSLR | Seth Zost et al., 2020 |
| | | | | | | 2262 | | V | (https://www.nature.com/ |
| | | | | | | | | | articles/s41591-020- |
| | | | | | | | | | 0998-x) |
| | | | | | | COV2- | 3648 | QQLNSYPV | Seth Zost et al., 2020 |
| | | | | | | 2263 | | T | (https://www.nature.com/ |
| | | | | | | | | | articles/s41591-020- |
| | | | | | | | | | 0998-x) |
| | | | | | | COV2- | 3649 | CSYAGSYT | Seth Zost et al., 2020 |
| | | | | | | 2266 | | YV | (https://www.nature.com/ |
| | | | | | | | | | articles/s41591-020- |
| | | | | | | | | | 0998-x) |
| | | | | | | COV2- | 3650 | SSYTSSSTL | Seth Zost et al., 2020 |
| | | | | | | 2268 | | VL | (https://www.nature.com/ |
| | | | | | | | | | articles/s41591-020- |
| | | | | | | | | | 0998-x) |
| | | | | | | COV2- | 3651 | QQRSNWP | Seth Zost et al., 2020 |
| | | | | | | 2270 | | PSYT | (https://www.nature.com/ |
| | | | | | | | | | articles/s41591-020- |
| | | | | | | | | | 0998-x) |
| | | | | | | COV2- | 3652 | SSYTSISTW | Seth Zost et al., 2020 |
| | | | | | | 2273 | | V | (https://www.nature.com/ |
| | | | | | | | | | articles/s41591-020- |
| | | | | | | | | | 0998-x) |
| | | | | | | COV2- | 3653 | FSMDSSG | Seth Zost et al., 2020 |
| | | | | | | 2274 | | DLRV | (https://www.nature.com/ |
| | | | | | | | | | articles/s41591-020- |
| | | | | | | | | | 0998-x) |
| | | | | | | COV2- | 3654 | QQSYSTPG | Seth Zost et al., 2020 |
| | | | | | | 2277 | | LT | (https://www.nature.com/ |
| | | | | | | | | | articles/s41591-020- |
| | | | | | | | | | 0998-x) |
| | | | | | | COV2- | 3655 | ISYTSSRTL | Seth Zost et al., 2020 |
| | | | | | | 2281 | | V | (https://www.nature.com/ |
| | | | | | | | | | articles/s41591-020- |
| | | | | | | | | | 0998-x) |
| | | | | | | COV2- | 3656 | QVWDSSY | Seth Zost et al., 2020 |
| | | | | | | 2287 | | YHPW | (https://www.nature.com/ |
| | | | | | | | | | articles/s41591-020- |
| | | | | | | | | | 0998-x) |
| | | | | | | COV2- | 3657 | LVWDDSL | Seth Zost et al., 2020 |
| | | | | | | 2290 | | NGLV | (https://www.nature.com/ |
| | | | | | | | | | articles/s41591-020- |
| | | | | | | | | | 0998-x) |
| | | | | | | COV2- | 3658 | GTWDSSLS | Seth Zost et al., 2020 |
| | | | | | | 2293 | | AW | (https://www.nature.com/ |
| | | | | | | | | | articles/s41591-020- |
| | | | | | | | | | 0998-x) |
| | | | | | | COV2- | 3659 | QQYSIYSW | Seth Zost et al., 2020 |
| | | | | | | 2296 | | T | (https://www.nature.com/ |
| | | | | | | | | | articles/s41591-020- |
| | | | | | | | | | 0998-x) |
| | | | | | | COV2- | 3660 | QQSYSTP | Seth Zost et al., 2020 |
| | | | | | | 2299 | | WT | (https://www.nature.com/ |
| | | | | | | | | | articles/s41591-020- |
| | | | | | | | | | 0998-x) |
| | | | | | | COV2- | 3661 | QQANSLPL | Seth Zost et al., 2020 |
| | | | | | | 2300 | | T | (https://www.nature.com/ |
| | | | | | | | | | articles/s41591-020- |
| | | | | | | | | | 0998-x) |
| | | | | | | COV2- | 3662 | AAWDDSL | Seth Zost et al., 2020 |
| | | | | | | 2304 | | NGW | (https://www.nature.com/ |
| | | | | | | | | | articles/s41591-020- |
| | | | | | | | | | 0998-x) |
| | | | | | | COV2- | 3663 | AAWDDSL | Seth Zost et al., 2020 |
| | | | | | | 2305 | | NGWV | (https://www.nature.com/ |
| | | | | | | | | | articles/s41591-020- |
| | | | | | | | | | 0998-x) |
| | | | | | | COV2- | 3664 | QAWDSST | Seth Zost et al., 2020 |
| | | | | | | 2307 | | AV | (https://www.nature.com/ |
| | | | | | | | | | articles/s41591-020- |
| | | | | | | | | | 0998-x) |
| | | | | | | COV2- | 3665 | HQYNNWP | Seth Zost et al., 2020 |
| | | | | | | 2308 | | QT | (https://www.nature.com/ |
| | | | | | | | | | articles/s41591-020- |
| | | | | | | | | | 0998-x) |
| | | | | | | COV2- | 3666 | QQSYSTLT | Seth Zost et al., 2020 |
| | | | | | | 2310 | | | (https://www.nature.com/ |
| | | | | | | | | | articles/s41591-020- |
| | | | | | | | | | 0998-x) |
| | | | | | | COV2- | 3667 | QHRSNWP | Seth Zost et al., 2020 |
| | | | | | | 2313 | | PRLT | (https://www.nature.com/ |
| | | | | | | | | | articles/s41591-020- |
| | | | | | | | | | 0998-x) |
| | | | | | | COV2- | 3668 | QQSYSTPG | Seth Zost et al., 2020 |
| | | | | | | 2318 | | T | (https://www.nature.com/ |
| | | | | | | | | | articles/s41591-020- |
| | | | | | | | | | 0998-x) |
| | | | | | | COV2- | 3669 | QQYGSSYT | Seth Zost et al., 2020 |
| | | | | | | 2322 | | | (https://www.nature.com/ |
| | | | | | | | | | articles/s41591-020- |
| | | | | | | | | | 0998-x) |
| | | | | | | COV2- | 3670 | QQTYSTF | Seth Zost et al., 2020 |
| | | | | | | 2325 | | WT | (https://www.nature.com/ |
| | | | | | | | | | articles/s41591-020- |
| | | | | | | | | | 0998-x) |
| | | | | | | COV2- | 3671 | AAWDDSL | Seth Zost et al., 2020 |
| | | | | | | 2329 | | SSWV | (https://www.nature.com/ |
| | | | | | | | | | articles/s41591-020- |
| | | | | | | | | | 0998-x) |
| | | | | | | COV2- | 3672 | QSYDSGNP | Seth Zost et al., 2020 |
| | | | | | | 2331 | | | (https://www.nature.com/ |
| | | | | | | | | | articles/s41591-020- |
| | | | | | | | | | 0998-x) |
| | | | | | | COV2- | 3673 | QAWSSST | Seth Zost et al., 2020 |
| | | | | | | 2333 | | AV | (https://www.nature.com/ |
| | | | | | | | | | articles/s41591-020- |
| | | | | | | | | | 0998-x) |
| | | | | | | COV2- | 3674 | SSYTSSTLN | Seth Zost et al., 2020 |
| | | | | | | 2335 | | VL | (https://www.nature.com/ |
| | | | | | | | | | articles/s41591-020- |
| | | | | | | | | | 0998-x) |
| | | | | | | COV2- | 3675 | QQSYSTPG | Seth Zost et al., 2020 |
| | | | | | | 2337 | | T | (https://www.nature.com/ |
| | | | | | | | | | articles/s41591-020- |
| | | | | | | | | | 0998-x) |
| | | | | | | COV2- | 3676 | QSYDSSLS | Seth Zost et al., 2020 |
| | | | | | | 2340 | | GWSV | (https://www.nature.com/ |
| | | | | | | | | | articles/s41591-020- |
| | | | | | | | | | 0998-x) |
| | | | | | | COV2- | 3677 | SSYTSSSTL | Seth Zost et al., 2020 |
| | | | | | | 2341 | | LYV | (https://www.nature.com/ |
| | | | | | | | | | articles/s41591-020- |
| | | | | | | | | | 0998-x) |
| | | | | | | COV2- | 3678 | QQSYSTH | Seth Zost et al., 2020 |
| | | | | | | 2342 | | MST | (https://www.nature.com/ |
| | | | | | | | | | articles/s41591-020- |
| | | | | | | | | | 0998-x) |
| | | | | | | COV2- | 3679 | MQSIQLA | Seth Zost et al., 2020 |
| | | | | | | 2343 | | | (https://www.nature.com/ |
| | | | | | | | | | articles/s41591-020- |
| | | | | | | | | | 0998-x) |
| | | | | | | COV2- | 3680 | QQYNYWP | Seth Zost et al., 2020 |
| | | | | | | 2346 | | PLT | (https://www.nature.com/ |
| | | | | | | | | | articles/s41591-020- |
| | | | | | | | | | 0998-x) |
| | | | | | | COV2- | 3681 | SSGDSSTD | Seth Zost et al., 2020 |
| | | | | | | 2351 | | HHVV | (https://www.nature.com/ |
| | | | | | | | | | articles/s41591-020- |
| | | | | | | | | | 0998-x) |
| | | | | | | COV2- | 3682 | HQSYGVPI | Seth Zost et al., 2020 |
| | | | | | | 2352 | | T | (https://www.nature.com/ |
| | | | | | | | | | articles/s41591-020- |
| | | | | | | | | | 0998-x) |
| | | | | | | COV2- | 3683 | QQYNNWP | Seth Zost et al., 2020 |
| | | | | | | 2353 | | PMYT | (https://www.nature.com/ |
| | | | | | | | | | articles/s41591-020- |
| | | | | | | | | | 0998-x) |
| | | | | | | COV2- | 3684 | QSYDSSKY | Seth Zost et al., 2020 |
| | | | | | | 2354 | | W | (https://www.nature.com/ |
| | | | | | | | | | articles/s41591-020- |
| | | | | | | | | | 0998-x) |
| | | | | | | COV2- | 3685 | QQYGSSP | Seth Zost et al., 2020 |
| | | | | | | 2355 | | WT | (https://www.nature.com/ |
| | | | | | | | | | articles/s41591-020- |
| | | | | | | | | | 0998-x) |
| | | | | | | COV2- | 3686 | MQALQTP | Seth Zost et al., 2020 |
| | | | | | | 2357 | | LYT | (https://www.nature.com/ |
| | | | | | | | | | articles/s41591-020- |
| | | | | | | | | | 0998-x) |
| | | | | | | COV2- | 3687 | QQSYSSLSI | Seth Zost et al., 2020 |
| | | | | | | 2358 | | T | (https://www.nature.com/ |
| | | | | | | | | | articles/s41591-020- |
| | | | | | | | | | 0998-x) |
| | | | | | | COV2- | 3688 | HKRSNWP | Seth Zost et al., 2020 |
| | | | | | | 2367 | | PSLT | (https://www.nature.com/ |
| | | | | | | | | | articles/s41591-020- |
| | | | | | | | | | 0998-x) |
| | | | | | | COV2- | 3689 | QSTASSGT | Seth Zost et al., 2020 |
| | | | | | | 2368 | | YVV | (https://www.nature.com/ |
| | | | | | | | | | articles/s41591-020- |
| | | | | | | | | | 0998-x) |
| | | | | | | COV2- | 3690 | QQYNSYSP | Seth Zost et al., 2020 |
| | | | | | | 2369 | | T | (https://www.nature.com/ |
| | | | | | | | | | articles/s41591-020- |
| | | | | | | | | | 0998-x) |
| | | | | | | COV2- | 3691 | QSYDGINR | Seth Zost et al., 2020 |
| | | | | | | 2370 | | WLV | (https://www.nature.com/ |
| | | | | | | | | | articles/s41591-020- |
| | | | | | | | | | 0998-x) |
| | | | | | | COV2- | 3692 | QQYNNWP | Seth Zost et al., 2020 |
| | | | | | | 2371 | | GT | (https://www.nature.com/ |
| | | | | | | | | | articles/s41591-020- |
| | | | | | | | | | 0998-x) |
| | | | | | | COV2- | 3693 | QQYGSSPP | Seth Zost et al., 2020 |
| | | | | | | 2373 | | | (https://www.nature.com/ |
| | | | | | | | | | articles/s41591-020- |
| | | | | | | | | | 0998-x) |
| | | | | | | COV2- | 3694 | GADHGSG | Seth Zost et al., 2020 |
| | | | | | | 2378 | | SNFVYVV | (https://www.nature.com/ |
| | | | | | | | | | articles/s41591-020- |
| | | | | | | | | | 0998-x) |
| | | | | | | COV2- | 3695 | QHFGSSSQ | Seth Zost et al., 2020 |
| | | | | | | 2381 | | WT | (https://www.nature.com/ |
| | | | | | | | | | articles/s41591-020- |
| | | | | | | | | | 0998-x) |
| | | | | | | COV2- | 3696 | LLYYGGPW | Seth Zost et al., 2020 |
| | | | | | | 2382 | | V | (https://www.nature.com/ |
| | | | | | | | | | articles/s41591-020- |
| | | | | | | | | | 0998-x) |
| | | | | | | COV2- | 3697 | QQYNNWP | Seth Zost et al., 2020 |
| | | | | | | 2383 | | LA | (https://www.nature.com/ |
| | | | | | | | | | articles/s41591-020- |
| | | | | | | | | | 0998-x) |
| | | | | | | COV2- | 3698 | CSYAGGNT | Seth Zost et al., 2020 |
| | | | | | | 2384 | | FVV | (https://www.nature.com/ |
| | | | | | | | | | articles/s41591-020- |
| | | | | | | | | | 0998-x) |
| | | | | | | COV2- | 3699 | QQYNNWP | Seth Zost et al., 2020 |
| | | | | | | 2386 | | RT | (https://www.nature.com/ |
| | | | | | | | | | articles/s41591-020- |
| | | | | | | | | | 0998-x) |
| | | | | | | COV2- | 3700 | SSYTSSSTP | Seth Zost et al., 2020 |
| | | | | | | 2387 | | W | (https://www.nature.com/ |
| | | | | | | | | | articles/s41591-020- |
| | | | | | | | | | 0998-x) |
| | | | | | | COV2- | 3701 | MQALQTP | Seth Zost et al., 2020 |
| | | | | | | 2388 | | RT | (https://www.nature.com/ |
| | | | | | | | | | articles/s41591-020- |
| | | | | | | | | | 0998-x) |
| | | | | | | COV2- | 3702 | QQSYSTP | Seth Zost et al., 2020 |
| | | | | | | 2389 | | WT | (https://www.nature.com/ |
| | | | | | | | | | articles/s41591-020- |
| | | | | | | | | | 0998-x) |
| | | | | | | COV2- | 3703 | AVWDDSL | Seth Zost et al., 2020 |
| | | | | | | 2391 | | NGLV | (https://www.nature.com/ |
| | | | | | | | | | articles/s41591-020- |
| | | | | | | | | | 0998-x) |
| | | | | | | COV2- | 3704 | QQCYNWP | Seth Zost et al., 2020 |
| | | | | | | 2394 | | PWT | (https://www.nature.com/ |
| | | | | | | | | | articles/s41591-020- |
| | | | | | | | | | 0998-x) |
| | | | | | | COV2- | 3705 | QAWDSST | Seth Zost et al., 2020 |
| | | | | | | 2397 | | GV | (https://www.nature.com/ |
| | | | | | | | | | articles/s41591-020- |
| | | | | | | | | | 0998-x) |
| | | | | | | COV2- | 3706 | QQYHNLP | Seth Zost et al., 2020 |
| | | | | | | 2399 | | RT | (https://www.nature.com/ |
| | | | | | | | | | articles/s41591-020- |
| | | | | | | | | | 0998-x) |
| | | | | | | COV2- | 3707 | QQLNSYPF | Seth Zost et al., 2020 |
| | | | | | | 2400 | | T | (https://www.nature.com/ |
| | | | | | | | | | articles/s41591-020- |
| | | | | | | | | | 0998-x) |
| | | | | | | COV2- | 3708 | QQYYSISW | Seth Zost et al., 2020 |
| | | | | | | 2401 | | T | (https://www.nature.com/ |
| | | | | | | | | | articles/s41591-020- |
| | | | | | | | | | 0998-x) |
| | | | | | | COV2- | 3709 | SSYTSSSTL | Seth Zost et al., 2020 |
| | | | | | | 2403 | | YV | (https://www.nature.com/ |
| | | | | | | | | | articles/s41591-020- |
| | | | | | | | | | 0998-x) |
| | | | | | | COV2- | 3710 | QKYNSAP | Seth Zost et al., 2020 |
| | | | | | | 2405 | | WT | (https://www.nature.com/ |
| | | | | | | | | | articles/s41591-020- |
| | | | | | | | | | 0998-x) |
| | | | | | | COV2- | 3711 | QAWDSST | Seth Zost et al., 2020 |
| | | | | | | 2406 | | GV | (https://www.nature.com/ |
| | | | | | | | | | articles/s41591-020- |
| | | | | | | | | | 0998-x) |
| | | | | | | COV2- | 3712 | QQLNSYPI | Seth Zost et al., 2020 |
| | | | | | | 2408 | | T | (https://www.nature.com/ |
| | | | | | | | | | articles/s41591-020- |
| | | | | | | | | | 0998-x) |
| | | | | | | COV2- | 3713 | AVWDDSL | Seth Zost et al., 2020 |
| | | | | | | 2413 | | NGLV | (https://www.nature.com/ |
| | | | | | | | | | articles/s41591-020- |
| | | | | | | | | | 0998-x) |
| | | | | | | COV2- | 3714 | AAWDDSL | Seth Zost et al., 2020 |
| | | | | | | 2416 | | NGW | (https://www.nature.com/ |
| | | | | | | | | | articles/s41591-020- |
| | | | | | | | | | 0998-x) |
| | | | | | | COV2- | 3715 | QQSYTTLL | Seth Zost et al., 2020 |
| | | | | | | 2417 | | T | (https://www.nature.com/ |
| | | | | | | | | | articles/s41591-020- |
| | | | | | | | | | 0998-x) |
| | | | | | | COV2- | 3716 | SSYTSTSTP | Seth Zost et al., 2020 |
| | | | | | | 2418 | | WV | (https://www.nature.com/ |
| | | | | | | | | | articles/s41591-020- |
| | | | | | | | | | 0998-x) |
| | | | | | | COV2- | 3717 | QQYHVWP | Seth Zost et al., 2020 |
| | | | | | | 2420 | | PIT | (https://www.nature.com/ |
| | | | | | | | | | articles/s41591-020- |
| | | | | | | | | | 0998-x) |
| | | | | | | COV2- | 3718 | QQYYSTPL | Seth Zost et al., 2020 |
| | | | | | | 2422 | | T | (https://www.nature.com/ |
| | | | | | | | | | articles/s41591-020- |
| | | | | | | | | | 0998-x) |
| | | | | | | COV2- | 3719 | QQGNSFPL | Seth Zost et al., 2020 |
| | | | | | | 2427 | | T | (https://www.nature.com/ |
| | | | | | | | | | articles/s41591-020- |
| | | | | | | | | | 0998-x) |
| | | | | | | COV2- | 3720 | QSYDSSLS | Seth Zost et al., 2020 |
| | | | | | | 2428 | | AWV | (https://www.nature.com/ |
| | | | | | | | | | articles/s41591-020- |
| | | | | | | | | | 0998-x) |
| | | | | | | COV2- | 3721 | QQYNTYS | Seth Zost et al., 2020 |
| | | | | | | 2429 | | QT | (https://www.nature.com/ |
| | | | | | | | | | articles/s41591-020- |
| | | | | | | | | | 0998-x) |
| | | | | | | COV2- | 3722 | QQRSNWP | Seth Zost et al., 2020 |
| | | | | | | 2430 | | PGVT | (https://www.nature.com/ |
| | | | | | | | | | articles/s41591-020- |
| | | | | | | | | | 0998-x) |
| | | | | | | COV2- | 3723 | HQYDYLPY | Seth Zost et al., 2020 |
| | | | | | | 2434 | | T | (https://www.nature.com/ |
| | | | | | | | | | articles/s41591-020- |
| | | | | | | | | | 0998-x) |
| | | | | | | COV2- | 3724 | QQTYSTPL | Seth Zost et al., 2020 |
| | | | | | | 2438 | | | (https://www.nature.com/ |
| | | | | | | | | | articles/s41591-020- |
| | | | | | | | | | 0998-x) |
| | | | | | | COV2- | 3725 | QHRTNWP | Seth Zost et al., 2020 |
| | | | | | | 2441 | | PLFT | (https://www.nature.com/ |
| | | | | | | | | | articles/s41591-020- |
| | | | | | | | | | 0998-x) |
| | | | | | | COV2- | 3726 | QSADSSGT | Seth Zost et al., 2020 |
| | | | | | | 2444 | | YVV | (https://www.nature.com/ |
| | | | | | | | | | articles/s41591-020- |
| | | | | | | | | | 0998-x) |
| | | | | | | COV2- | 3727 | QQYNTYSG | Seth Zost et al., 2020 |
| | | | | | | 2445 | | T | (https://www.nature.com/ |
| | | | | | | | | | articles/s41591-020- |
| | | | | | | | | | 0998-x) |
| | | | | | | COV2- | 3728 | GADHGSG | Seth Zost et al., 2020 |
| | | | | | | 2446 | | SNFVFVV | (https://www.nature.com/ |
| | | | | | | | | | articles/s41591-020- |
| | | | | | | | | | 0998-x) |
| | | | | | | COV2- | 3729 | QQYYSPP | Seth Zost et al., 2020 |
| | | | | | | 2449 | | WT | (https://www.nature.com/ |
| | | | | | | | | | articles/s41591-020- |
| | | | | | | | | | 0998-x) |
| | | | | | | COV2- | 3730 | QQYHNLP | Seth Zost et al., 2020 |
| | | | | | | 2450 | | PIT | (https://www.nature.com/ |
| | | | | | | | | | articles/s41591-020- |
| | | | | | | | | | 0998-x) |
| | | | | | | COV2- | 3731 | QAWDSST | Seth Zost et al., 2020 |
| | | | | | | 2451 | | GGV | (https://www.nature.com/ |
| | | | | | | | | | articles/s41591-020- |
| | | | | | | | | | 0998-x) |
| | | | | | | COV2- | 3732 | QQYDNLP | Seth Zost et al., 2020 |
| | | | | | | 2453 | | PGVSTT | (https://www.nature.com/ |
| | | | | | | | | | articles/s41591-020- |
| | | | | | | | | | 0998-x) |
| | | | | | | COV2- | 3733 | CSTDSSGN | Seth Zost et al., 2020 |
| | | | | | | 2454 | | QRV | (https://www.nature.com/ |
| | | | | | | | | | articles/s41591-020- |
| | | | | | | | | | 0998-x) |
| | | | | | | COV2- | 3734 | SSYTSSSTH | Seth Zost et al., 2020 |
| | | | | | | 2455 | | W | (https://www.nature.com/ |
| | | | | | | | | | articles/s41591-020- |
| | | | | | | | | | 0998-x) |
| | | | | | | COV2- | 3735 | QSADTIGT | Seth Zost et al., 2020 |
| | | | | | | 2458 | | YWV | (https://www.nature.com/ |
| | | | | | | | | | articles/s41591-020- |
| | | | | | | | | | 0998-x) |
| | | | | | | COV2- | 3736 | QQYNNWP | Seth Zost et al., 2020 |
| | | | | | | 2459 | | PMYT | (https://www.nature.com/ |
| | | | | | | | | | articles/s41591-020- |
| | | | | | | | | | 0998-x) |
| | | | | | | COV2- | 3737 | QQYDNLYS | Seth Zost et al., 2020 |
| | | | | | | 2461 | | VH | (https://www.nature.com/ |
| | | | | | | | | | articles/s41591-020- |
| | | | | | | | | | 0998-x) |
| | | | | | | COV2- | 3738 | QQSYEMP | Seth Zost et al., 2020 |
| | | | | | | 2462 | | PWT | (https://www.nature.com/ |
| | | | | | | | | | articles/s41591-020- |
| | | | | | | | | | 0998-x) |
| | | | | | | COV2- | 3739 | QSADSSGY | Seth Zost et al., 2020 |
| | | | | | | 2464 | | V | (https://www.nature.com/ |
| | | | | | | | | | articles/s41591-020- |
| | | | | | | | | | 0998-x) |
| | | | | | | COV2- | 3740 | SSYTSSSIPY | Seth Zost et al., 2020 |
| | | | | | | 2465 | | V | (https://www.nature.com/ |
| | | | | | | | | | articles/s41591-020- |
| | | | | | | | | | 0998-x) |
| | | | | | | COV2- | 3741 | QQRGNW | Seth Zost et al., 2020 |
| | | | | | | 2466 | | WT | (https://www.nature.com/ |
| | | | | | | | | | articles/s41591-020- |
| | | | | | | | | | 0998-x) |
| | | | | | | COV2- | 3742 | YSTDTSGN | Seth Zost et al., 2020 |
| | | | | | | 2473 | | HWV | (https://www.nature.com/ |
| | | | | | | | | | articles/s41591-020- |
| | | | | | | | | | 0998-x) |
| | | | | | | COV2- | 3743 | YSTDSSGN | Seth Zost et al., 2020 |
| | | | | | | 2474 | | HRV | (https://www.nature.com/ |
| | | | | | | | | | articles/s41591-020- |
| | | | | | | | | | 0998-x) |
| | | | | | | COV2- | 3744 | HQYGTSPY | Seth Zost et al., 2020 |
| | | | | | | 2478 | | T | (https://www.nature.com/ |
| | | | | | | | | | articles/s41591-020- |
| | | | | | | | | | 0998-x) |
| | | | | | | COV2- | 3745 | QQYNNFLT | Seth Zost et al., 2020 |
| | | | | | | 2479 | | | (https://www.nature.com/ |
| | | | | | | | | | articles/s41591-020- |
| | | | | | | | | | 0998-x) |
| | | | | | | COV2- | 3746 | QQYYSTPG | Seth Zost et al., 2020 |
| | | | | | | 2481 | | | (https://www.nature.com/ |
| | | | | | | | | | articles/s41591-020- |
| | | | | | | | | | 0998-x) |
| | | | | | | COV2- | 3747 | QSYDSSLS | Seth Zost et al., 2020 |
| | | | | | | 2485 | | DSV | (https://www.nature.com/ |
| | | | | | | | | | articles/s41591-020- |
| | | | | | | | | | 0998-x) |
| | | | | | | COV2- | 3748 | QSADSSGT | Seth Zost et al., 2020 |
| | | | | | | 2488 | | SWV | (https://www.nature.com/ |
| | | | | | | | | | articles/s41591-020- |
| | | | | | | | | | 0998-x) |
| | | | | | | COV2- | 3749 | QQYGSSPF | Seth Zost et al., 2020 |
| | | | | | | 2489 | | T | (https://www.nature.com/ |
| | | | | | | | | | articles/s41591-020- |
| | | | | | | | | | 0998-x) |
| | | | | | | COV2- | 3750 | QQYNSYSL | Seth Zost et al., 2020 |
| | | | | | | 2490 | | T | (https://www.nature.com/ |
| | | | | | | | | | articles/s41591-020- |
| | | | | | | | | | 0998-x) |
| | | | | | | COV2- | 3751 | QQYNNW | Seth Zost et al., 2020 |
| | | | | | | 2495 | | WRT | (https://www.nature.com/ |
| | | | | | | | | | articles/s41591-020- |
| | | | | | | | | | 0998-x) |
| | | | | | | COV2- | 3752 | YSTDSSGN | Seth Zost et al., 2020 |
| | | | | | | 2496 | | HWV | (https://www.nature.com/ |
| | | | | | | | | | articles/s41591-020- |
| | | | | | | | | | 0998-x) |
| | | | | | | COV2- | 3753 | NFRDSSGH | Seth Zost et al., 2020 |
| | | | | | | 2499 | | HPV | (https://www.nature.com/ |
| | | | | | | | | | articles/s41591-020- |
| | | | | | | | | | 0998-x) |
| | | | | | | COV2- | 3754 | AAWDDSL | Seth Zost et al., 2020 |
| | | | | | | 2504 | | NALV | (https://www.nature.com/ |
| | | | | | | | | | articles/s41591-020- |
| | | | | | | | | | 0998-x) |
| | | | | | | COV2- | 3755 | QQYNNWP | Seth Zost et al., 2020 |
| | | | | | | 2509 | | GT | (https://www.nature.com/ |
| | | | | | | | | | articles/s41591-020- |
| | | | | | | | | | 0998-x) |
| | | | | | | COV2- | 3756 | YSAADNN | Seth Zost et al., 2020 |
| | | | | | | 2510 | | RV | (https://www.nature.com/ |
| | | | | | | | | | articles/s41591-020- |
| | | | | | | | | | 0998-x) |
| | | | | | | COV2- | 3757 | NSRDSSGN | Seth Zost et al., 2020 |
| | | | | | | 2514 | | AW | (https://www.nature.com/ |
| | | | | | | | | | articles/s41591-020- |
| | | | | | | | | | 0998-x) |
| | | | | | | COV2- | 3758 | QQYDNLLL | Seth Zost et al., 2020 |
| | | | | | | 2515 | | | (https://www.nature.com/ |
| | | | | | | | | | articles/s41591-020- |
| | | | | | | | | | 0998-x) |
| | | | | | | COV2- | 3759 | QQRSNWP | Seth Zost et al., 2020 |
| | | | | | | 2516 | | PRTWT | (https://www.nature.com/ |
| | | | | | | | | | articles/s41591-020- |
| | | | | | | | | | 0998-x) |
| | | | | | | COV2- | 3760 | HYRSNWP | Seth Zost et al., 2020 |
| | | | | | | 2517 | | PVLT | (https://www.nature.com/ |
| | | | | | | | | | articles/s41591-020- |
| | | | | | | | | | 0998-x) |
| | | | | | | COV2- | 3761 | NSRDSSGN | Seth Zost et al., 2020 |
| | | | | | | 2518 | | AW | (https://www.nature.com/ |
| | | | | | | | | | articles/s41591-020- |
| | | | | | | | | | 0998-x) |
| | | | | | | COV2- | 3762 | NSRDSSGY | Seth Zost et al., 2020 |
| | | | | | | 2520 | | IWGWM | (https://www.nature.com/ |
| | | | | | | | | | articles/s41591-020- |
| | | | | | | | | | 0998-x) |
| | | | | | | COV2- | 3763 | QVWDSSS | Seth Zost et al., 2020 |
| | | | | | | 2521 | | DHWV | (https://www.nature.com/ |
| | | | | | | | | | articles/s41591-020- |
| | | | | | | | | | 0998-x) |
| | | | | | | COV2- | 3764 | QSYDSSLG | Seth Zost et al., 2020 |
| | | | | | | 2524 | | AL | (https://www.nature.com/ |
| | | | | | | | | | articles/s41591-020- |
| | | | | | | | | | 0998-x) |
| | | | | | | COV2- | 3765 | QSWDTGI | Seth Zost et al., 2020 |
| | | | | | | 2525 | | GV | (https://www.nature.com/ |
| | | | | | | | | | articles/s41591-020- |
| | | | | | | | | | 0998-x) |
| | | | | | | COV2- | 3766 | QAWDSST | Seth Zost et al., 2020 |
| | | | | | | 2526 | | W | (https://www.nature.com/ |
| | | | | | | | | | articles/s41591-020- |
| | | | | | | | | | 0998-x) |
| | | | | | | COV2- | 3767 | CSYVGSST | Seth Zost et al., 2020 |
| | | | | | | 2527 | | YVV | (https://www.nature.com/ |
| | | | | | | | | | articles/s41591-020- |
| | | | | | | | | | 0998-x) |
| | | | | | | COV2- | 3768 | QSADSSSH | Seth Zost et al., 2020 |
| | | | | | | 2529 | | WV | (https://www.nature.com/ |
| | | | | | | | | | articles/s41591-020- |
| | | | | | | | | | 0998-x) |
| | | | | | | COV2- | 3769 | QSFDSGNV | Seth Zost et al., 2020 |
| | | | | | | 2531 | | V | (https://www.nature.com/ |
| | | | | | | | | | articles/s41591-020- |
| | | | | | | | | | 0998-x) |
| | | | | | | COV2- | 3770 | VAWDDSR | Seth Zost et al., 2020 |
| | | | | | | 2532 | | NGLV | (https://www.nature.com/ |
| | | | | | | | | | articles/s41591-020- |
| | | | | | | | | | 0998-x) |
| | | | | | | COV2- | 3771 | QQYYSTRT | Seth Zost et al., 2020 |
| | | | | | | 2533 | | | (https://www.nature.com/ |
| | | | | | | | | | articles/s41591-020- |
| | | | | | | | | | 0998-x) |
| | | | | | | COV2- | 3772 | QQYYSTPP | Seth Zost et al., 2020 |
| | | | | | | 2536 | | | (https://www.nature.com/ |
| | | | | | | | | | articles/s41591-020- |
| | | | | | | | | | 0998-x) |
| | | | | | | COV2- | 3773 | LAWDDSL | Seth Zost et al., 2020 |
| | | | | | | 2539 | | NGLV | (https://www.nature.com/ |
| | | | | | | | | | articles/s41591-020- |
| | | | | | | | | | 0998-x) |
| | | | | | | COV2- | 3774 | AAWDASL | Seth Zost et al., 2020 |
| | | | | | | 2545 | | SGWV | (https://www.nature.com/ |
| | | | | | | | | | articles/s41591-020- |
| | | | | | | | | | 0998-x) |
| | | | | | | COV2- | 3775 | QSVDGSGS | Seth Zost et al., 2020 |
| | | | | | | 2546 | | SVV | (https://www.nature.com/ |
| | | | | | | | | | articles/s41591-020- |
| | | | | | | | | | 0998-x) |
| | | | | | | COV2- | 3776 | QAWDSST | Seth Zost et al., 2020 |
| | | | | | | 2549 | | HVV | (https://www.nature.com/ |
| | | | | | | | | | articles/s41591-020- |
| | | | | | | | | | 0998-x) |
| | | | | | | COV2- | 3777 | SSYTSSSTP | Seth Zost et al., 2020 |
| | | | | | | 2551 | | FV | (https://www.nature.com/ |
| | | | | | | | | | articles/s41591-020- |
| | | | | | | | | | 0998-x) |
| | | | | | | COV2- | 3778 | ETWDSNT | Seth Zost et al., 2020 |
| | | | | | | 2552 | | RV | (https://www.nature.com/ |
| | | | | | | | | | articles/s41591-020- |
| | | | | | | | | | 0998-x) |
| | | | | | | COV2- | 3779 | QQSYSTP | Seth Zost et al., 2020 |
| | | | | | | 2553 | | MHT | (https://www.nature.com/ |
| | | | | | | | | | articles/s41591-020- |
| | | | | | | | | | 0998-x) |
| | | | | | | COV2- | 3780 | QQLNSYLG | Seth Zost et al., 2020 |
| | | | | | | 2554 | | T | (https://www.nature.com/ |
| | | | | | | | | | articles/s41591-020- |
| | | | | | | | | | 0998-x) |
| | | | | | | COV2- | 3781 | QQANSFPP | Seth Zost et al., 2020 |
| | | | | | | 2558 | | T | (https://www.nature.com/ |
| | | | | | | | | | articles/s41591-020- |
| | | | | | | | | | 0998-x) |
| | | | | | | COV2- | 3782 | LVWDDSL | Seth Zost et al., 2020 |
| | | | | | | 2562 | | NGLV | (https://www.nature.com/ |
| | | | | | | | | | articles/s41591-020- |
| | | | | | | | | | 0998-x) |
| | | | | | | COV2- | 3783 | CSYAGSST | Seth Zost et al., 2020 |
| | | | | | | 2563 | | WG | (https://www.nature.com/ |
| | | | | | | | | | articles/s41591-020- |
| | | | | | | | | | 0998-x) |
| | | | | | | COV2- | 3784 | QVWDSSS | Seth Zost et al., 2020 |
| | | | | | | 2564 | | DHHW | (https://www.nature.com/ |
| | | | | | | | | | articles/s41591-020- |
| | | | | | | | | | 0998-x) |
| | | | | | | COV2- | 3785 | MQALQTP | Seth Zost et al., 2020 |
| | | | | | | 2565 | | LT | (https://www.nature.com/ |
| | | | | | | | | | articles/s41591-020- |
| | | | | | | | | | 0998-x) |
| | | | | | | COV2- | 3786 | QQYNNWP | Seth Zost et al., 2020 |
| | | | | | | 2570 | | PMYT | (https://www.nature.com/ |
| | | | | | | | | | articles/s41591-020- |
| | | | | | | | | | 0998-x) |
| | | | | | | COV2- | 3787 | QQYNNWP | Seth Zost et al., 2020 |
| | | | | | | 2571 | | RT | (https://www.nature.com/ |
| | | | | | | | | | articles/s41591-020- |
| | | | | | | | | | 0998-x) |
| | | | | | | COV2- | 3788 | QTWGT | Seth Zost et al., 2020 |
| | | | | | | 2574 | | | (https://www.nature.com/ |
| | | | | | | | | | articles/s41591-020- |
| | | | | | | | | | 0998-x) |
| | | | | | | COV2- | 3789 | MQSIQPPL | Seth Zost et al., 2020 |
| | | | | | | 2582 | | T | (https://www.nature.com/ |
| | | | | | | | | | articles/s41591-020- |
| | | | | | | | | | 0998-x) |
| | | | | | | COV2- | 3790 | QQYYSTPL | Seth Zost et al., 2020 |
| | | | | | | 2583 | | T | (https://www.nature.com/ |
| | | | | | | | | | articles/s41591-020- |
| | | | | | | | | | 0998-x) |
| | | | | | | COV2- | 3791 | SSFTSRGAL | Seth Zost et al., 2020 |
| | | | | | | 2584 | | VL | (https://www.nature.com/ |
| | | | | | | | | | articles/s41591-020- |
| | | | | | | | | | 0998-x) |
| | | | | | | COV2- | 3792 | CSYAGIWV | Seth Zost et al., 2020 |
| | | | | | | 2585 | | | (https://www.nature.com/ |
| | | | | | | | | | articles/s41591-020- |
| | | | | | | | | | 0998-x) |
| | | | | | | COV2- | 3793 | QHYGSSRS | Seth Zost et al., 2020 |
| | | | | | | 2586 | | T | (https://www.nature.com/ |
| | | | | | | | | | articles/s41591-020- |
| | | | | | | | | | 0998-x) |
| | | | | | | COV2- | 3794 | QQSYSTPT | Seth Zost et al., 2020 |
| | | | | | | 2587 | | | (https://www.nature.com/ |
| | | | | | | | | | articles/s41591-020- |
| | | | | | | | | | 0998-x) |
| | | | | | | COV2- | 3795 | QQSYSTPY | Seth Zost et al., 2020 |
| | | | | | | 2589 | | T | (https://www.nature.com/ |
| | | | | | | | | | articles/s41591-020- |
| | | | | | | | | | 0998-x) |
| | | | | | | COV2- | 3796 | NSRDSSGN | Seth Zost et al., 2020 |
| | | | | | | 2590 | | HLRV | (https://www.nature.com/ |
| | | | | | | | | | articles/s41591-020- |
| | | | | | | | | | 0998-x) |
| | | | | | | COV2- | 3797 | QQYGRSPI | Seth Zost et al., 2020 |
| | | | | | | 2602 | | T | (https://www.nature.com/ |
| | | | | | | | | | articles/s41591-020- |
| | | | | | | | | | 0998-x) |
| | | | | | | COV2- | 3798 | QQYDNLL | Seth Zost et al., 2020 |
| | | | | | | 2610 | | QFT | (https://www.nature.com/ |
| | | | | | | | | | articles/s41591-020- |
| | | | | | | | | | 0998-x) |
| | | | | | | COV2- | 3799 | YSTDSSGN | Seth Zost et al., 2020 |
| | | | | | | 2611 | | V | (https://www.nature.com/ |
| | | | | | | | | | articles/s41591-020- |
| | | | | | | | | | 0998-x) |
| | | | | | | COV2- | 3800 | QQYNSYSL | Seth Zost et al., 2020 |
| | | | | | | 2614 | | T | (https://www.nature.com/ |
| | | | | | | | | | articles/s41591-020- |
| | | | | | | | | | 0998-x) |
| | | | | | | COV2- | 3801 | QYYGSSPF | Seth Zost et al., 2020 |
| | | | | | | 2616 | | G | (https://www.nature.com/ |
| | | | | | | | | | articles/s41591-020- |
| | | | | | | | | | 0998-x) |
| | | | | | | COV2- | 3802 | QQRSNWP | Seth Zost et al., 2020 |
| | | | | | | 2617 | | PRLT | (https://www.nature.com/ |
| | | | | | | | | | articles/s41591-020- |
| | | | | | | | | | 0998-x) |
| | | | | | | COV2- | 3803 | AVWDDSL | Seth Zost et al., 2020 |
| | | | | | | 2618 | | NGVV | (https://www.nature.com/ |
| | | | | | | | | | articles/s41591-020- |
| | | | | | | | | | 0998-x) |
| | | | | | | COV2- | 3804 | LVWDDSL | Seth Zost et al., 2020 |
| | | | | | | 2619 | | NGLV | (https://www.nature.com/ |
| | | | | | | | | | articles/s41591-020- |
| | | | | | | | | | 0998-x) |
| | | | | | | COV2- | 3805 | SSYTSSRAV | Seth Zost et al., 2020 |
| | | | | | | 2620 | | L | (https://www.nature.com/ |
| | | | | | | | | | articles/s41591-020- |
| | | | | | | | | | 0998-x) |
| | | | | | | COV2- | 3806 | QQYYSSH | Seth Zost et al., 2020 |
| | | | | | | 2621 | | WT | (https://www.nature.com/ |
| | | | | | | | | | articles/s41591-020- |
| | | | | | | | | | 0998-x) |
| | | | | | | COV2- | 3807 | YSYAGSST | Seth Zost et al., 2020 |
| | | | | | | 2622 | | WV | (https://www.nature.com/ |
| | | | | | | | | | articles/s41591-020- |
| | | | | | | | | | 0998-x) |
| | | | | | | COV2- | 3808 | QSTDSSGS | Seth Zost et al., 2020 |
| | | | | | | 2624 | | YVV | (https://www.nature.com/ |
| | | | | | | | | | articles/s41591-020- |
| | | | | | | | | | 0998-x) |
| | | | | | | COV2- | 3809 | HQSYFTPQ | Seth Zost et al., 2020 |
| | | | | | | 2628 | | T | (https://www.nature.com/ |
| | | | | | | | | | articles/s41591-020- |
| | | | | | | | | | 0998-x) |
| | | | | | | COV2- | 3810 | QQYGNSP | Seth Zost et al., 2020 |
| | | | | | | 2631 | | | (https://www.nature.com/ |
| | | | | | | | | | articles/s41591-020- |
| | | | | | | | | | 0998-x) |
| | | | | | | COV2- | 3811 | QQYVEPPF | Seth Zost et al., 2020 |
| | | | | | | 2632 | | T | (https://www.nature.com/ |
| | | | | | | | | | articles/s41591-020- |
| | | | | | | | | | 0998-x) |
| | | | | | | COV2- | 3812 | SAYAGSNN | Seth Zost et al., 2020 |
| | | | | | | 2639 | | LV | (https://www.nature.com/ |
| | | | | | | | | | articles/s41591-020- |
| | | | | | | | | | 0998-x) |
| | | | | | | COV2- | 3813 | QQYGSSYT | Seth Zost et al., 2020 |
| | | | | | | 2641 | | | (https://www.nature.com/ |
| | | | | | | | | | articles/s41591-020- |
| | | | | | | | | | 0998-x) |
| | | | | | | COV2- | 3814 | QQSYSTPG | Seth Zost et al., 2020 |
| | | | | | | 2643 | | T | (https://www.nature.com/ |
| | | | | | | | | | articles/s41591-020- |
| | | | | | | | | | 0998-x) |
| | | | | | | COV2- | 3815 | QQYGRSS | Seth Zost et al., 2020 |
| | | | | | | 2656 | | GT | (https://www.nature.com/ |
| | | | | | | | | | articles/s41591-020- |
| | | | | | | | | | 0998-x) |
| | | | | | | COV2- | 3816 | QQYGSSPL | Seth Zost et al., 2020 |
| | | | | | | 2660 | | IT | (https://www.nature.com/ |
| | | | | | | | | | articles/s41591-020- |
| | | | | | | | | | 0998-x) |
| | | | | | | COV2- | 3817 | QQYYTAPL | Seth Zost et al., 2020 |
| | | | | | | 2669 | | T | (https://www.nature.com/ |
| | | | | | | | | | articles/s41591-020- |
| | | | | | | | | | 0998-x) |
| | | | | | | COV2- | 3818 | LLYYGGA | Seth Zost et al., 2020 |
| | | | | | | 2673 | | WV | (https://www.nature.com/ |
| | | | | | | | | | articles/s41591-020- |
| | | | | | | | | | 0998-x) |
| | | | | | | COV2- | 3819 | HQSSSLPP | Seth Zost et al., 2020 |
| | | | | | | 2675 | | T | (https://www.nature.com/ |
| | | | | | | | | | articles/s41591-020- |
| | | | | | | | | | 0998-x) |
| | | | | | | COV2- | 3820 | QRRSNWP | Seth Zost et al., 2020 |
| | | | | | | 2676 | | PFT | (https://www.nature.com/ |
| | | | | | | | | | articles/s41591-020- |
| | | | | | | | | | 0998-x) |
| | | | | | | COV2- | 3821 | QSYDSSNY | Seth Zost et al., 2020 |
| | | | | | | 2677 | | WV | (https://www.nature.com/ |
| | | | | | | | | | articles/s41591-020- |
| | | | | | | | | | 0998-x) |
| | | | | | | COV2- | 3822 | NSRDSSGN | Seth Zost et al., 2020 |
| | | | | | | 2678 | | AW | (https://www.nature.com/ |
| | | | | | | | | | articles/s41591-020- |
| | | | | | | | | | 0998-x) |
| | | | | | | COV2- | 3823 | QQYYSSPY | Seth Zost et al., 2020 |
| | | | | | | 2681 | | T | (https://www.nature.com/ |
| | | | | | | | | | articles/s41591-020- |
| | | | | | | | | | 0998-x) |
| | | | | | | COV2- | 3824 | QQYNNW | Seth Zost et al., 2020 |
| | | | | | | 2684 | | WRT | (https://www.nature.com/ |
| | | | | | | | | | articles/s41591-020- |
| | | | | | | | | | 0998-x) |
| | | | | | | COV2- | 3825 | QQYGSSPR | Seth Zost et al., 2020 |
| | | | | | | 2685 | | LT | (https://www.nature.com/ |
| | | | | | | | | | articles/s41591-020- |
| | | | | | | | | | 0998-x) |
| | | | | | | COV2- | 3826 | AVWDDSL | Seth Zost et al., 2020 |
| | | | | | | 2693 | | NGLV | (https://www.nature.com/ |
| | | | | | | | | | articles/s41591-020- |
| | | | | | | | | | 0998-x) |
| | | | | | | COV2- | 3827 | QSYDSSLN | Seth Zost et al., 2020 |
| | | | | | | 2694 | | GDV | (https://www.nature.com/ |
| | | | | | | | | | articles/s41591-020- |
| | | | | | | | | | 0998-x) |
| | | | | | | COV2- | 3828 | QQRSNWP | Seth Zost et al., 2020 |
| | | | | | | 2697 | | LIFT | (https://www.nature.com/ |
| | | | | | | | | | articles/s41591-020- |
| | | | | | | | | | 0998-x) |
| | | | | | | COV2- | 3829 | QSYDSSLS | Seth Zost et al., 2020 |
| | | | | | | 2700 | | GYV | (https://www.nature.com/ |
| | | | | | | | | | articles/s41591-020- |
| | | | | | | | | | 0998-x) |
| | | | | | | COV2- | 3830 | QQSYSTPPI | Seth Zost et al., 2020 |
| | | | | | | 2703 | | T | (https://www.nature.com/ |
| | | | | | | | | | articles/s41591-020- |
| | | | | | | | | | 0998-x) |
| | | | | | | COV2- | 3831 | QQSYSTPP | Seth Zost et al., 2020 |
| | | | | | | 2705 | | WT | (https://www.nature.com/ |
| | | | | | | | | | articles/s41591-020- |
| | | | | | | | | | 0998-x) |
| | | | | | | COV2- | 3832 | QQYANLPF | Seth Zost et al., 2020 |
| | | | | | | 2709 | | T | (https://www.nature.com/ |
| | | | | | | | | | articles/s41591-020- |
| | | | | | | | | | 0998-x) |
| | | | | | | COV2- | 3833 | VAWDDSR | Seth Zost et al., 2020 |
| | | | | | | 2710 | | NGLV | (https://www.nature.com/ |
| | | | | | | | | | articles/s41591-020- |
| | | | | | | | | | 0998-x) |
| | | | | | | COV2- | 3834 | QSVDSSGT | Seth Zost et al., 2020 |
| | | | | | | 2713 | | YPHVI | (https://www.nature.com/ |
| | | | | | | | | | articles/s41591-020- |
| | | | | | | | | | 0998-x) |
| | | | | | | COV2- | 3835 | QQSYSTPY | Seth Zost et al., 2020 |
| | | | | | | 2717 | | T | (https://www.nature.com/ |
| | | | | | | | | | articles/s41591-020- |
| | | | | | | | | | 0998-x) |
| | | | | | | COV2- | 3836 | QQLNSYPL | Seth Zost et al., 2020 |
| | | | | | | 2718 | | T | (https://www.nature.com/ |
| | | | | | | | | | articles/s41591-020- |
| | | | | | | | | | 0998-x) |
| | | | | | | COV2- | 3837 | TSYTSSSTL | Seth Zost et al., 2020 |
| | | | | | | 2722 | | NW | (https://www.nature.com/ |
| | | | | | | | | | articles/s41591-020- |
| | | | | | | | | | 0998-x) |
| | | | | | | COV2- | 3838 | CSYASSSIV | Seth Zost et al., 2020 |
| | | | | | | 2726 | | V | (https://www.nature.com/ |
| | | | | | | | | | articles/s41591-020- |
| | | | | | | | | | 0998-x) |
| | | | | | | COV2- | 3839 | QQSYSTPP | Seth Zost et al., 2020 |
| | | | | | | 2730 | | WT | (https://www.nature.com/ |
| | | | | | | | | | articles/s41591-020- |
| | | | | | | | | | 0998-x) |
| | | | | | | COV2- | 3840 | QQLNSYSF | Seth Zost et al., 2020 |
| | | | | | | 2733 | | ET | (https://www.nature.com/ |
| | | | | | | | | | articles/s41591-020- |
| | | | | | | | | | 0998-x) |
| | | | | | | COV2- | 3841 | QSYDGINR | Seth Zost et al., 2020 |
| | | | | | | 2734 | | WLV | (https://www.nature.com/ |
| | | | | | | | | | articles/s41591-020- |
| | | | | | | | | | 0998-x) |
| | | | | | | COV2- | 3842 | QQYNSYPY | Seth Zost et al., 2020 |
| | | | | | | 2736 | | T | (https://www.nature.com/ |
| | | | | | | | | | articles/s41591-020- |
| | | | | | | | | | 0998-x) |
| | | | | | | COV2- | 3843 | QQSSSSPIT | Seth Zost et al., 2020 |
| | | | | | | 2740 | | | (https://www.nature.com/ |
| | | | | | | | | | articles/s41591-020- |
| | | | | | | | | | 0998-x) |
| | | | | | | COV2- | 3844 | QQSYTTLL | Seth Zost et al., 2020 |
| | | | | | | 2749 | | T | (https://www.nature.com/ |
| | | | | | | | | | articles/s41591-020- |
| | | | | | | | | | 0998-x) |
| | | | | | | COV2- | 3845 | QVWDGIN | Seth Zost et al., 2020 |
| | | | | | | 2751 | | DRW | (https://www.nature.com/ |
| | | | | | | | | | articles/s41591-020- |
| | | | | | | | | | 0998-x) |
| | | | | | | COV2- | 3846 | QQYDNLP | Seth Zost et al., 2020 |
| | | | | | | 2752 | | PV | (https://www.nature.com/ |
| | | | | | | | | | articles/s41591-020- |
| | | | | | | | | | 0998-x) |
| | | | | | | COV2- | 3847 | QQSYSSE | Seth Zost et al., 2020 |
| | | | | | | 2753 | | WT | (https://www.nature.com/ |
| | | | | | | | | | articles/s41591-020- |
| | | | | | | | | | 0998-x) |
| | | | | | | COV2- | 3848 | QSYDISLG | Seth Zost et al., 2020 |
| | | | | | | 2756 | | GWV | (https://www.nature.com/ |
| | | | | | | | | | articles/s41591-020- |
| | | | | | | | | | 0998-x) |
| | | | | | | COV2- | 3849 | QQSYSMP | Seth Zost et al., 2020 |
| | | | | | | 2758 | | PIT | (https://www.nature.com/ |
| | | | | | | | | | articles/s41591-020- |
| | | | | | | | | | 0998-x) |
| | | | | | | COV2- | 3850 | AAWDDSL | Seth Zost et al., 2020 |
| | | | | | | 2759 | | NGSWV | (https://www.nature.com/ |
| | | | | | | | | | articles/s41591-020- |
| | | | | | | | | | 0998-x) |
| | | | | | | COV2- | 3851 | AAWDDSL | Seth Zost et al., 2020 |
| | | | | | | 2760 | | SGLI | (https://www.nature.com/ |
| | | | | | | | | | articles/s41591-020- |
| | | | | | | | | | 0998-x) |
| | | | | | | COV2- | 3852 | CSYAGSST | Seth Zost et al., 2020 |
| | | | | | | 2762 | | WL | (https://www.nature.com/ |
| | | | | | | | | | articles/s41591-020- |
| | | | | | | | | | 0998-x) |
| | | | | | | COV2- | 3853 | QVWDSSS | Seth Zost et al., 2020 |
| | | | | | | 2765 | | DHHW | (https://www.nature.com/ |
| | | | | | | | | | articles/s41591-020- |
| | | | | | | | | | 0998-x) |
| | | | | | | COV2- | 3854 | VAWDDSR | Seth Zost et al., 2020 |
| | | | | | | 2767 | | NGLV | (https://www.nature.com/ |
| | | | | | | | | | articles/s41591-020- |
| | | | | | | | | | 0998-x) |
| | | | | | | COV2- | 3855 | GTWDSSLS | Seth Zost et al., 2020 |
| | | | | | | 2768 | | AYV | (https://www.nature.com/ |
| | | | | | | | | | articles/s41591-020- |
| | | | | | | | | | 0998-x) |
| | | | | | | COV2- | 3856 | QVWDSSS | Seth Zost et al., 2020 |
| | | | | | | 2769 | | DHPGV | (https://www.nature.com/ |
| | | | | | | | | | articles/s41591-020- |
| | | | | | | | | | 0998-x) |
| | | | | | | COV2- | 3857 | QAWGSSR | Seth Zost et al., 2020 |
| | | | | | | 2774 | | GV | (https://www.nature.com/ |
| | | | | | | | | | articles/s41591-020- |
| | | | | | | | | | 0998-x) |
| | | | | | | COV2- | 3858 | NSRDNSG | Seth Zost et al., 2020 |
| | | | | | | 2776 | | NLNWV | (https://www.nature.com/ |
| | | | | | | | | | articles/s41591-020- |
| | | | | | | | | | 0998-x) |
| | | | | | | COV2- | 3859 | QQYDNLPY | Seth Zost et al., 2020 |
| | | | | | | 2780 | | T | (https://www.nature.com/ |
| | | | | | | | | | articles/s41591-020- |
| | | | | | | | | | 0998-x) |
| | | | | | | COV2- | 3860 | QSADSSGS | Seth Zost et al., 2020 |
| | | | | | | 2783 | | R | (https://www.nature.com/ |
| | | | | | | | | | articles/s41591-020- |
| | | | | | | | | | 0998-x) |
| | | | | | | COV2- | 3861 | QQYNNWP | Seth Zost et al., 2020 |
| | | | | | | 2784 | | | (https://www.nature.com/ |
| | | | | | | | | | articles/s41591-020- |
| | | | | | | | | | 0998-x) |
| | | | | | | COV2- | 3862 | QQSYEIPP | Seth Zost et al., 2020 |
| | | | | | | 2786 | | WT | (https://www.nature.com/ |
| | | | | | | | | | articles/s41591-020- |
| | | | | | | | | | 0998-x) |
| | | | | | | COV2- | 3863 | ATWDDSL | Seth Zost et al., 2020 |
| | | | | | | 2789 | | NGPV | (https://www.nature.com/ |
| | | | | | | | | | articles/s41591-020- |
| | | | | | | | | | 0998-x) |
| | | | | | | COV2- | 3864 | QSYDSRLS | Seth Zost et al., 2020 |
| | | | | | | 2790 | | GFW | (https://www.nature.com/ |
| | | | | | | | | | articles/s41591-020- |
| | | | | | | | | | 0998-x) |
| | | | | | | COV2- | 3865 | QSADSSGT | Seth Zost et al., 2020 |
| | | | | | | 2794 | | SVL | (https://www.nature.com/ |
| | | | | | | | | | articles/s41591-020- |
| | | | | | | | | | 0998-x) |
| | | | | | | COV2- | 3866 | CSYARSSTR | Seth Zost et al., 2020 |
| | | | | | | 2796 | | V | (https://www.nature.com/ |
| | | | | | | | | | articles/s41591-020- |
| | | | | | | | | | 0998-x) |
| | | | | | | COV2- | 3867 | QSADSRG | Seth Zost et al., 2020 |
| | | | | | | 2797 | | AV | (https://www.nature.com/ |
| | | | | | | | | | articles/s41591-020- |
| | | | | | | | | | 0998-x) |
| | | | | | | COV2- | 3868 | AVWDDSL | Seth Zost et al., 2020 |
| | | | | | | 2801 | | HSYV | (https://www.nature.com/ |
| | | | | | | | | | articles/s41591-020- |
| | | | | | | | | | 0998-x) |
| | | | | | | COV2- | 3869 | QQSYSTPR | Seth Zost et al., 2020 |
| | | | | | | 2807 | | T | (https://www.nature.com/ |
| | | | | | | | | | articles/s41591-020- |
| | | | | | | | | | 0998-x) |
| | | | | | | COV2- | 3870 | QQYDNLPL | Seth Zost et al., 2020 |
| | | | | | | 2808 | | | (https://www.nature.com/ |
| | | | | | | | | | articles/s41591-020- |
| | | | | | | | | | 0998-x) |
| | | | | | | COV2- | 3871 | QQSYTTLL | Seth Zost et al., 2020 |
| | | | | | | 2809 | | T | (https://www.nature.com/ |
| | | | | | | | | | articles/s41591-020- |
| | | | | | | | | | 0998-x) |
| | | | | | | COV2- | 3872 | QSYDSSLS | Seth Zost et al., 2020 |
| | | | | | | 2811 | | GWV | (https://www.nature.com/ |
| | | | | | | | | | articles/s41591-020- |
| | | | | | | | | | 0998-x) |
| | | | | | | COV2- | 3873 | QQYANLPF | Seth Zost et al., 2020 |
| | | | | | | 2812 | | T | (https://www.nature.com/ |
| | | | | | | | | | articles/s41591-020- |
| | | | | | | | | | 0998-x) |
| | | | | | | COV2- | 3874 | QQYDNLP | Seth Zost et al., 2020 |
| | | | | | | 2813 | | RT | (https://www.nature.com/ |
| | | | | | | | | | articles/s41591-020- |
| | | | | | | | | | 0998-x) |
| | | | | | | COV2- | 3875 | QSYDSSLS | Seth Zost et al., 2020 |
| | | | | | | 2814 | | GYV | (https://www.nature.com/ |
| | | | | | | | | | articles/s41591-020- |
| | | | | | | | | | 0998-x) |
| | | | | | | COV2- | 3876 | CSYAVSTTY | Seth Zost et al., 2020 |
| | | | | | | 2816 | | VI | (https://www.nature.com/ |
| | | | | | | | | | articles/s41591-020- |
| | | | | | | | | | 0998-x) |
| | | | | | | COV2- | 3877 | QQSYSNPS | Seth Zost et al., 2020 |
| | | | | | | 2817 | | | (https://www.nature.com/ |
| | | | | | | | | | articles/s41591-020- |
| | | | | | | | | | 0998-x) |
| | | | | | | COV2- | 3878 | QQYGSSPP | Seth Zost et al., 2020 |
| | | | | | | 2818 | | RYT | (https://www.nature.com/ |
| | | | | | | | | | articles/s41591-020- |
| | | | | | | | | | 0998-x) |
| | | | | | | COV2- | 3879 | QQSYSTLL | Seth Zost et al., 2020 |
| | | | | | | 2819 | | | (https://www.nature.com/ |
| | | | | | | | | | articles/s41591-020- |
| | | | | | | | | | 0998-x) |
| | | | | | | COV2- | 3880 | MQALQTP | Seth Zost et al., 2020 |
| | | | | | | 2820 | | FT | (https://www.nature.com/ |
| | | | | | | | | | articles/s41591-020- |
| | | | | | | | | | 0998-x) |
| | | | | | | COV2- | 3881 | QVWDSSS | Seth Zost et al., 2020 |
| | | | | | | 2821 | | DRLYV | (https://www.nature.com/ |
| | | | | | | | | | articles/s41591-020- |
| | | | | | | | | | 0998-x) |
| | | | | | | COV2- | 3882 | QQSYSNPS | Seth Zost et al., 2020 |
| | | | | | | 2822 | | | (https://www.nature.com/ |
| | | | | | | | | | articles/s41591-020- |
| | | | | | | | | | 0998-x) |
| | | | | | | COV2- | 3883 | CSYAGSST | Seth Zost et al., 2020 |
| | | | | | | 2826 | | WL | (https://www.nature.com/ |
| | | | | | | | | | articles/s41591-020- |
| | | | | | | | | | 0998-x) |
| | | | | | | COV2- | 3884 | QSYDSTSR | Seth Zost et al., 2020 |
| | | | | | | 2828 | | W | (https://www.nature.com/ |
| | | | | | | | | | articles/s41591-020- |
| | | | | | | | | | 0998-x) |
| | | | | | | COV2- | 3885 | QVWDSGS | Seth Zost et al., 2020 |
| | | | | | | 2830 | | DHVV | (https://www.nature.com/ |
| | | | | | | | | | articles/s41591-020- |
| | | | | | | | | | 0998-x) |
| | | | | | | COV2- | 3886 | QQSYSTPQ | Seth Zost et al., 2020 |
| | | | | | | 2832 | | T | (https://www.nature.com/ |
| | | | | | | | | | articles/s41591-020- |
| | | | | | | | | | 0998-x) |
| | | | | | | COV2- | 3887 | ASWDDSLI | Seth Zost et al., 2020 |
| | | | | | | 2834 | | GPV | (https://www.nature.com/ |
| | | | | | | | | | articles/s41591-020- |
| | | | | | | | | | 0998-x) |
| | | | | | | COV2- | 3888 | QQLNSYPG | Seth Zost et al., 2020 |
| | | | | | | 2835 | | YT | (https://www.nature.com/ |
| | | | | | | | | | articles/s41591-020- |
| | | | | | | | | | 0998-x) |
| | | | | | | COV2- | 3889 | QSYDSNN | Seth Zost et al., 2020 |
| | | | | | | 2841 | | QV | (https://www.nature.com/ |
| | | | | | | | | | articles/s41591-020- |
| | | | | | | | | | 0998-x) |
| | | | | | | COV2- | 3890 | QSYDSSDV | Seth Zost et al., 2020 |
| | | | | | | 2842 | | V | (https://www.nature.com/ |
| | | | | | | | | | articles/s41591-020- |
| | | | | | | | | | 0998-x) |
| | | | | | | COV2- | 3891 | QVWDSSS | Seth Zost et al., 2020 |
| | | | | | | 2844 | | DHHW | (https://www.nature.com/ |
| | | | | | | | | | articles/s41591-020- |
| | | | | | | | | | 0998-x) |
| | | | | | | COV2- | 3892 | QSADSSGT | Seth Zost et al., 2020 |
| | | | | | | 2848 | | YRV | (https://www.nature.com/ |
| | | | | | | | | | articles/s41591-020- |
| | | | | | | | | | 0998-x) |
| | | | | | | COV2- | 3893 | QQYYNWP | Seth Zost et al., 2020 |
| | | | | | | 2853 | | LT | (https://www.nature.com/ |
| | | | | | | | | | articles/s41591-020- |
| | | | | | | | | | 0998-x) |
| | | | | | | COV2- | 3894 | QSADSSAA | Seth Zost et al., 2020 |
| | | | | | | 2863 | | YVV | (https://www.nature.com/ |
| | | | | | | | | | articles/s41591-020- |
| | | | | | | | | | 0998-x) |
| | | | | | | COV2- | 3895 | YSTDSSGK | Seth Zost et al., 2020 |
| | | | | | | 2872 | | GV | (https://www.nature.com/ |
| | | | | | | | | | articles/s41591-020- |
| | | | | | | | | | 0998-x) |
| | | | | | | COV2- | 3896 | QVWDSSS | Seth Zost et al., 2020 |
| | | | | | | 2873 | | DPFYV | (https://www.nature.com/ |
| | | | | | | | | | articles/s41591-020- |
| | | | | | | | | | 0998-x) |
| | | | | | | COV2- | 3897 | QSYDGINR | Seth Zost et al., 2020 |
| | | | | | | 2878 | | AWV | (https://www.nature.com/ |
| | | | | | | | | | articles/s41591-020- |
| | | | | | | | | | 0998-x) |
| | | | | | | COV2- | 3898 | QQYYSTP | Seth Zost et al., 2020 |
| | | | | | | 2883 | | WT | (https://www.nature.com/ |
| | | | | | | | | | articles/s41591-020- |
| | | | | | | | | | 0998-x) |
| | | | | | | COV2- | 3899 | QQYNSYSY | Seth Zost et al., 2020 |
| | | | | | | 2891 | | T | (https://www.nature.com/ |
| | | | | | | | | | articles/s41591-020- |
| | | | | | | | | | 0998-x) |
| | | | | | | COV2- | 3900 | QQYDDWP | Seth Zost et al., 2020 |
| | | | | | | 2894 | | PEVT | (https://www.nature.com/ |
| | | | | | | | | | articles/s41591-020- |
| | | | | | | | | | 0998-x) |
| | | | | | | COV2- | 3901 | VAWDDSR | Seth Zost et al., 2020 |
| | | | | | | 2901 | | NGLV | (https://www.nature.com/ |
| | | | | | | | | | articles/s41591-020- |
| | | | | | | | | | 0998-x) |
| | | | | | | COV2- | 3902 | QSFDIGRG | Seth Zost et al., 2020 |
| | | | | | | 2904 | | GWI | (https://www.nature.com/ |
| | | | | | | | | | articles/s41591-020- |
| | | | | | | | | | 0998-x) |
| | | | | | | COV2- | 3903 | QQSYSTPP | Seth Zost et al., 2020 |
| | | | | | | 2906 | | T | (https://www.nature.com/ |
| | | | | | | | | | articles/s41591-020- |
| | | | | | | | | | 0998-x) |
| | | | | | | COV2- | 3904 | QSYDSSLS | Seth Zost et al., 2020 |
| | | | | | | 2909 | | GSV | (https://www.nature.com/ |
| | | | | | | | | | articles/s41591-020- |
| | | | | | | | | | 0998-x) |
| | | | | | | COV2- | 3905 | CSYAGSVL | Seth Zost et al., 2020 |
| | | | | | | 2911 | | | (https://www.nature.com/ |
| | | | | | | | | | articles/s41591-020- |
| | | | | | | | | | 0998-x) |
| | | | | | | COV2- | 3906 | QQSFSTPR | Seth Zost et al., 2020 |
| | | | | | | 2919 | | T | (https://www.nature.com/ |
| | | | | | | | | | articles/s41591-020- |
| | | | | | | | | | 0998-x) |
| | | | | | | COV2- | 3907 | QVWDSSS | Seth Zost et al., 2020 |
| | | | | | | 2933 | | DHPEWV | (https://www.nature.co |
| | | | | | | | | | m/articles/s41591-020- |
| | | | | | | | | | 0998-x) |
| | | | | | | COV2- | 3908 | MQALQTP | Seth Zost et al., 2020 |
| | | | | | | 2934 | | WT | (https://www.nature.com/ |
| | | | | | | | | | articles/s41591-020- |
| | | | | | | | | | 0998-x) |
| | | | | | | COV2- | 3909 | NSRDSSGN | Seth Zost et al., 2020 |
| | | | | | | 2939 | | PRW | (https://www.nature.com/ |
| | | | | | | | | | articles/s41591-020- |
| | | | | | | | | | 0998-x) |
| | | | | | | COV2- | 3910 | QQYGSSPP | Seth Zost et al., 2020 |
| | | | | | | 2941 | | RYT | (https://www.nature.com/ |
| | | | | | | | | | articles/s41591-020- |
| | | | | | | | | | 0998-x) |
| | | | | | | COV2- | 3911 | QSVDSSGT | Seth Zost et al., 2020 |
| | | | | | | 2942 | | YRV | (https://www.nature.com/ |
| | | | | | | | | | articles/s41591-020- |
| | | | | | | | | | 0998-x) |
| | | | | | | COV2- | 3912 | QQYDNLPL | Seth Zost et al., 2020 |
| | | | | | | 2944 | | | (https://www.nature.com/ |
| | | | | | | | | | articles/s41591-020- |
| | | | | | | | | | 0998-x) |
| | | | | | | COV2- | 3913 | MQGTHW | Seth Zost et al., 2020 |
| | | | | | | 2945 | | PYT | (https://www.nature.com/ |
| | | | | | | | | | articles/s41591-020- |
| | | | | | | | | | 0998-x) |
| | | | | | | COV2- | 3914 | CSYAGRYT | Seth Zost et al., 2020 |
| | | | | | | 2947 | | WV | (https://www.nature.com/ |
| | | | | | | | | | articles/s41591-020- |
| | | | | | | | | | 0998-x) |
| | | | | | | COV2- | 3915 | QLLNSHPL | Seth Zost et al., 2020 |
| | | | | | | 2952 | | T | (https://www.nature.com/ |
| | | | | | | | | | articles/s41591-020- |
| | | | | | | | | | 0998-x) |
| | | | | | | COV2- | 3916 | QSADSTG | Seth Zost et al., 2020 |
| | | | | | | 2953 | | WV | (https://www.nature.com/ |
| | | | | | | | | | articles/s41591-020- |
| | | | | | | | | | 0998-x) |
| | | | | | | COV2- | 3917 | MQSIQLPY | Seth Zost et al., 2020 |
| | | | | | | 2955 | | T | (https://www.nature.com/ |
| | | | | | | | | | articles/s41591-020- |
| | | | | | | | | | 0998-x) |
| | | | | | | COV2- | 3918 | MQSIQLA | Seth Zost et al., 2020 |
| | | | | | | 2960 | | | (https://www.nature.com/ |
| | | | | | | | | | articles/s41591-020- |
| | | | | | | | | | 0998-x) |
| | | | | | | COV2- | 3919 | QHYGSSRG | Seth Zost et al., 2020 |
| | | | | | | 2961 | | WT | (https://www.nature.com/ |
| | | | | | | | | | articles/s41591-020- |
| | | | | | | | | | 0998-x) |
| | | | | | | COV2- | 3920 | QQYYNWP | Seth Zost et al., 2020 |
| | | | | | | 2997 | | LT | (https://www.nature.com/ |
| | | | | | | | | | articles/s41591-020- |
| | | | | | | | | | 0998-x) |
| | | | | | | COV2- | 3921 | QQYNSYPY | Seth Zost et al., 2020 |
| | | | | | | 3010 | | T | (https://www.nature.com/ |
| | | | | | | | | | articles/s41591-020- |
| | | | | | | | | | 0998-x) |
| | | | | | | COV2- | 3922 | QQSYEIPP | Seth Zost et al., 2020 |
| | | | | | | 3012 | | WT | (https://www.nature.com/ |
| | | | | | | | | | articles/s41591-020- |
| | | | | | | | | | 0998-x) |
| | | | | | | COV2- | 3923 | QQYANLPF | Seth Zost et al., 2020 |
| | | | | | | 3013 | | T | (https://www.nature.com/ |
| | | | | | | | | | articles/s41591-020- |
| | | | | | | | | | 0998-x) |
| | | | | | | COV2- | 3924 | QHYGSSRG | Seth Zost et al., 2020 |
| | | | | | | 3025 | | WT | (https://www.nature.com/ |
| | | | | | | | | | articles/s41591-020- |
| | | | | | | | | | 0998-x) |
| | | | | | | COV2- | 3925 | QSVDSSGT | Seth Zost et al., 2020 |
| | | | | | | 3029 | | YPHVI | (https://www.nature.com/ |
| | | | | | | | | | articles/s41591-020- |
| | | | | | | | | | 0998-x) |
| | | | | | | COV2- | 3926 | QSYASSLS | Seth Zost et al., 2020 |
| | | | | | | 3057 | | AHVV | (https://www.nature.com/ |
| | | | | | | | | | articles/s41591-020- |
| | | | | | | | | | 0998-x) |
| | | | | | | COV2- | 3927 | QQYYSSYT | Seth Zost et al., 2020 |
| | | | | | | 3058 | | | (https://www.nature.co |
| | | | | | | | | | m/articles/s41591-020- |
| | | | | | | | | | 0998-x) |
| | | | | | | COVA1- | 3928 | QQYGSSPP | Philip Brouwer et al., |
| | | | | | | 01 | | PFT | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3929 | SSYTSSSTP | Philip Brouwer et al., |
| | | | | | | 02 | | W | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3930 | QKYNSAPP | Philip Brouwer et al., |
| | | | | | | 03 | | | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3931 | QAWGSTT | Philip Brouwer et al., |
| | | | | | | 06 | | AKV | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3932 | QQRSNWP | Philip Brouwer et al., |
| | | | | | | 07 | | PRVT | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3933 | SSYTSSSTR | Philip Brouwer et al., |
| | | | | | | 08 | | HWV | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3934 | QQSYSTPY | Philip Brouwer et al., |
| | | | | | | 09 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3935 | SAYTTTSTS | Philip Brouwer et al., |
| | | | | | | 10 | | WV | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3936 | SSYVGNN | Philip Brouwer et al., |
| | | | | | | 12 | | NWV | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3937 | QQYDNPP | Philip Brouwer et al., |
| | | | | | | 16 | | LT | 2020 |
| | | | | | | | | | (https://science.sciencemag. |
| | | | | | | | | | org/content/early/ |
| | | | | | | | | | 2020/06/15/science.abc5902), |
| | | | | | | | | | Hejun Liu et al 2020 |
| | | | | | | | | | (https://www.biorxiv.or |
| | | | | | | | | | g/content/10.1101/ |
| | | | | | | | | | 2020.08.02.233536v1.full. |
| | | | | | | | | | pdf) |
| | | | | | | COVA1- | 3938 | LLSYSGVW | Philip Brouwer et al., |
| | | | | | | 18 | | V | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3939 | QQYNNWP | Philip Brouwer et al., |
| | | | | | | 19 | | PWT | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3940 | QVWDSSS | Philip Brouwer et al., |
| | | | | | | 20 | | DHWV | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3941 | QQYNNWP | Philip Brouwer et al., |
| | | | | | | 21 | | PGT | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3942 | QAWDSST | Philip Brouwer et al., |
| | | | | | | 22 | | AV | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3943 | QSADSSGT | Philip Brouwer et al., |
| | | | | | | 23 | | YSW | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3944 | QQYNSYSI | Philip Brouwer et al., |
| | | | | | | 25 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3945 | QQLHSYPL | Philip Brouwer et al., |
| | | | | | | 26 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA1- | 3946 | QQYGSSPL | Philip Brouwer et al., |
| | | | | | | 27 | | | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3947 | QQSYSTPP | Philip Brouwer et al., |
| | | | | | | 01 | | VT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3948 | QQSYSTH | Philip Brouwer et al., |
| | | | | | | 02 | | MST | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3949 | QQRSNWP | Philip Brouwer et al., |
| | | | | | | 03 | | QVT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3950 | QQYGSLYT | Philip Brouwer et al., |
| | | | | | | 04 | | | 2020 |
| | | | | | | | | | (https://science.sciencemag. |
| | | | | | | | | | org/content/early |
| | | | | | | | | | 2020/06/15/science.abc5902); |
| | | | | | | | | | Nicholas Wu |
| | | | | | | | | | (https://www.biorxiv.org/ |
| | | | | | | | | | content/10.1101/ |
| | | | | | | | | | 2020.07.26.222232v1) |
| | | | | | | COVA2- | 3951 | QQYDNLPL | Philip Brouwer et al., |
| | | | | | | 05 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3952 | QQYGSSP | Philip Brouwer et al., |
| | | | | | | 07 | | GT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3953 | QQYGSSLL | Philip Brouwer et al., |
| | | | | | | 10 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3954 | QQYGSSPR | Philip Brouwer et al., |
| | | | | | | 11 | | LT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3955 | QQYNNWP | Philip Brouwer et al., |
| | | | | | | 12 | | PWT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3956 | QQYGSSP | Philip Brouwer et al., |
| | | | | | | 13 | | GT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3957 | QQRSNWP | Philip Brouwer et al., |
| | | | | | | 14 | | PMYT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3958 | MQGTHW | Philip Brouwer et al., |
| | | | | | | 15 | | PRT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3959 | LQHNSYPP | Philip Brouwer et al., |
| | | | | | | 16 | | L | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3960 | QQRSNWP | Philip Brouwer et al., |
| | | | | | | 17 | | PYT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3961 | QQRSNWP | Philip Brouwer et al., |
| | | | | | | 18 | | PSIT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3962 | LQHNSYL | Philip Brouwer et al., |
| | | | | | | 20 | | WT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3963 | QQYDNLPI | Philip Brouwer et al., |
| | | | | | | 22 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3964 | QQYGSSP | Philip Brouwer et al., |
| | | | | | | 23 | | GVT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3965 | QQSYSTPQ | Philip Brouwer et al., |
| | | | | | | 24 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3966 | SSYTSSST | Philip Brouwer et al., |
| | | | | | | 25 | | WV | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3967 | QAWDSST | Philip Brouwer et al., |
| | | | | | | 26 | | AW | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3968 | QQYNYWP | Philip Brouwer et al., |
| | | | | | | 28 | | LIT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3969 | QQSYSTPR | Philip Brouwer et al., |
| | | | | | | 29 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3970 | QVWASSS | Philip Brouwer et al., |
| | | | | | | 30 | | W | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3971 | MQSIQLPP | Philip Brouwer et al., |
| | | | | | | 31 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3972 | QQRSNWP | Philip Brouwer et al., |
| | | | | | | 32 | | PRLT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3973 | CSYAGSV | Philip Brouwer et al., |
| | | | | | | 33 | | | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3974 | SSYTSSSTL | Philip Brouwer et al., |
| | | | | | | 34 | | GLYV | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3975 | QSYDSSLS | Philip Brouwer et al., |
| | | | | | | 37 | | GSV | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3976 | LQHNSYPL | Philip Brouwer et al., |
| | | | | | | 38 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3977 | CSYAGSST | Philip Brouwer et al., |
| | | | | | | 39 | | WV | 2020 |
| | | | | | | | | | (https://science.sciencemag. |
| | | | | | | | | | org/content/early/ |
| | | | | | | | | | 2020/06/15/science.abc5902); |
| | | | | | | | | | Nicholas Wu |
| | | | | | | | | | (https://www.biorxiv. |
| | | | | | | | | | org/content/10.1101/ |
| | | | | | | | | | 2020.07.26.222232v1) |
| | | | | | | COVA2- | 3978 | CSYAGSST | Philip Brouwer et al., |
| | | | | | | 40 | | WV | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3979 | QQCYNWP | Philip Brouwer et al., |
| | | | | | | 41 | | PWT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3980 | LQHNSYPL | Philip Brouwer et al., |
| | | | | | | 43 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3981 | QQSYTTFIY | Philip Brouwer et al., |
| | | | | | | 44 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3982 | QQYGSSPY | Philip Brouwer et al., |
| | | | | | | 45 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3983 | QQYDNLLS | Philip Brouwer et al., |
| | | | | | | 46 | | LT | 2020 |
| | | | | | | | | | (https://science.sciencemag. |
| | | | | | | | | | org/content/early/ |
| | | | | | | | | | 2020/06/15/science.abc5902) |
| | | | | | | COVA2- | 3984 | QQYYSTPP | Philip Brouwer et al., |
| | | | | | | 47 | | LT | 2020 |
| | | | | | | | | | (https://science.sciencemag. |
| | | | | | | | | | org/content/early/ |
| | | | | | | | | | 2020/06/15/science.abc5902) |
| | | | | | | COVA3- | 3985 | QQSYSTLT | Philip Brouwer et al., |
| | | | | | | 01 | | | 2020 |
| | | | | | | | | | (https://science.sciencemag. |
| | | | | | | | | | org/content/early/ |
| | | | | | | | | | 2020/06/15/science.abc5902) |
| | | | | | | COVA3- | 3986 | QSADSSGT | Philip Brouwer et al., |
| | | | | | | 03 | | YRV | 2020 |
| | | | | | | | | | (https://science.sciencemag. |
| | | | | | | | | | org/content/early/ |
| | | | | | | | | | 2020/06/15/science.abc5902) |
| | | | | | | COVA3- | 3987 | QVWDSSS | Philip Brouwer et al., |
| | | | | | | 04 | | DHYV | 2020 |
| | | | | | | | | | (https://science.sciencemag. |
| | | | | | | | | | org/content/early/ |
| | | | | | | | | | 2020/06/15/science.abc5902) |
| | | | | | | COVA3- | 3988 | AAWDDSL | Philip Brouwer et al., |
| | | | | | | 05 | | NGPHWV | 2020 |
| | | | | | | | | | (https://science.sciencemag. |
| | | | | | | | | | org/content/early/ |
| | | | | | | | | | 2020/06/15/science.abc5902) |
| | | | | | | COVA3- | 3989 | QQYYSTPL | Philip Brouwer et al., |
| | | | | | | 06 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA3- | 3990 | QQYGSSPF | Philip Brouwer et al., |
| | | | | | | 07 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA3- | 3991 | QQSYSTPL | Philip Brouwer et al., |
| | | | | | | 08 | | T | 2020 |
| | | | | | | | | | (https://science.sciencemag. |
| | | | | | | | | | org/content/early/ |
| | | | | | | | | | 2020/06/15/science.abc5902) |
| | | | | | | COVA3- | 3992 | QQSYSTLT | Philip Brouwer et al., |
| | | | | | | 09 | | | 2020 |
| | | | | | | | | | (https://science.sciencemag. |
| | | | | | | | | | org/content/early/ |
| | | | | | | | | | 2020/06/15/science.abc5902) |
| | | | | | | COVA3- | 3993 | QQYYSTPIT | Philip Brouwer et al., |
| | | | | | | 10 | | | 2020 |
| | | | | | | | | | (https://science.sciencemag. |
| | | | | | | | | | org/content/early/ |
| | | | | | | | | | 2020/06/15/science.abc5902) |
| | | | | | | CR3022 | 3994 | QQYYSTPY | Jan ter Meulen et al., |
| | | | | | | | | T | 2006 |
| | | | | | | | | | (https://journals.plos.org/ |
| | | | | | | | | | plosmedicine/article?id = |
| | | | | | | | | | 10.1371/journal.pmed. |
| | | | | | | | | | 003 0237); Meng Yuan |
| | | | | | | | | | et al., 2020a |
| | | | | | | | | | (https://science.sciencemag. |
| | | | | | | | | | org/content/early/ |
| | | | | | | | | | 2020/04/02/science.abb7269); |
| | | | | | | | | | Meng Yuan et al.,2020b |
| | | | | | | | | | (https://www.biorxiv. |
| | | | | | | | | | org/content/10.1101/ |
| | | | | | | | | | 2020.06.08.141267vl) |
| | | | | | | CV-X1- | 3995 | QQANGFP | Jakob Kreye et al., 2020 |
| | | | | | | 126 | | PL | (https://www.biorxiv.org/ |
| | | | | | | | | | content/10.1101/ |
| | | | | | | | | | 2020.08.15.252320v1.full. |
| | | | | | | | | | pdf) |
| | | | | | | CV-X2- | 3996 | QQSYSTPY | Jakob Kreye et al., 2020 |
| | | | | | | 106 | | T | (https://www.biorxiv.org/ |
| | | | | | | | | | content/10.1101/ |
| | | | | | | | | | 2020.08.15.252320v1.full. |
| | | | | | | | | | pdf) |
| | | | | | | CV05-163 | 3997 | QQRSNWP | Jakob Kreye et al., 2020 |
| | | | | | | | | PVT | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-200 | 3998 | SSYTSSSTY | Jakob Kreye et al., 2020 |
| | | | | | | | | V | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-209 | 3999 | QQYDNLPL | Jakob Kreye et al., 2020 |
| | | | | | | | | T | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-222 | 4000 | CSYAGGST | Jakob Kreye et al., 2020 |
| | | | | | | | | SYV | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-250 | 4001 | SSYAGNND | Jakob Kreye et al., 2020 |
| | | | | | | | | FV | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-255 | 4002 | CSYAGHST | Jakob Kreye et al., 2020 |
| | | | | | | | | WV | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-262 | 4003 | CSYAGTST | Jakob Kreye et al., 2020 |
| | | | | | | | | FV | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-270 | 4004 | CSYAGSSS | Jakob Kreye et al., 2020 |
| | | | | | | | | WV | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-283 | 4005 | SSYTSSSTY | Jakob Kreye et al., 2020 |
| | | | | | | | | V | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-287 | 4006 | QQYYGSSP | Jakob Kreye et al., 2020 |
| | | | | | | | | WT | (https://www.biorxiv.org/ |
| | | | | | | | | | content/10.1101/ |
| | | | | | | | | | 2020.08.15.252320v1.full. |
| | | | | | | | | | pdf) |
| | | | | | | CV07-315 | 4007 | QQSYSTHA | Jakob Kreye et al., 2020 |
| | | | | | | | | | (https://www.biorxiv.org/ |
| | | | | | | | | | content/10.1101/ |
| | | | | | | | | | 2020.08.15.252320v1.full. |
| | | | | | | | | | pdf) |
| | | | | | | CV1 | 4008 | AAWDDSL | Emilie Seydoux et al., |
| | | | | | | | | NGPV | 2020 |
| | | | | | | | | | (https://www.biorxiv.org/ |
| | | | | | | | | | content/10.1101/ |
| | | | | | | | | | 2020.05.12.091298v1) |
| | | | | | | CV10 | 4009 | QQYAGSP | Emilie Seydoux et al., |
| | | | | | | | | WT | 2020 |
| | | | | | | | | | (https://www.biorxiv.org/ |
| | | | | | | | | | content/10.1101/ |
| | | | | | | | | | 2020.05.12.091298v1) |
| | | | | | | CV11 | 4010 | QQRSNWP | Emilie Seydoux et al., |
| | | | | | | | | PIFT | 2020 |
| | | | | | | | | | (https://www.biorxiv.org/ |
| | | | | | | | | | content/10.1101/ |
| | | | | | | | | | 2020.05.12.091298v1) |
| | | | | | | CV12 | 4011 | MQGTHW | Emilie Seydoux et al., |
| | | | | | | | | PVT | 2020 |
| | | | | | | | | | (https://www.biorxiv.org/ |
| | | | | | | | | | content/10.1101/ |
| | | | | | | | | | 2020.05.12.091298v1) |
| | | | | | | CV13 | 4012 | QQSYSNPL | Emilie Seydoux et al., |
| | | | | | | | | T | 2020 |
| | | | | | | | | | (https://www.biorxiv.org/ |
| | | | | | | | | | content/10.1101/ |
| | | | | | | | | | 2020.05.12.091298v1) |
| | | | | | | CV15 | 4013 | CSYAGSYT | Emilie Seydoux et al., |
| | | | | | | | | WV | 2020 |
| | | | | | | | | | (https://www.biorxiv.org/ |
| | | | | | | | | | content/10.1101/ |
| | | | | | | | | | 2020.05.12.091298v1) |
| | | | | | | CV16 | 4014 | QQYGSSR | Emilie Seydoux et al., |
| | | | | | | | | GT | 2020 |
| | | | | | | | | | (https://www.biorxiv.org/ |
| | | | | | | | | | content/10.1101/ |
| | | | | | | | | | 2020.05.12.091298v1) |
| | | | | | | CV17 | 4015 | CSYAGSST | Emilie Seydoux et al., |
| | | | | | | | | YV | 2020 |
| | | | | | | | | | (https://www.biorxiv.org/ |
| | | | | | | | | | content/10.1101/ |
| | | | | | | | | | 2020.05.12.091298v1) |
| | | | | | | CV18 | 4016 | GTWDSSLS | Emilie Seydoux et al., |
| | | | | | | | | AGPV | 2020 |
| | | | | | | | | | (https://www.biorxiv.org/ |
| | | | | | | | | | content/10.1101/ |
| | | | | | | | | | 2020.05.12.091298v1) |
| | | | | | | CV19 | 4017 | QQYGSSPP | Emilie Seydoux et al., |
| | | | | | | | | KYT | 2020 |
| | | | | | | | | | (https://www.biorxiv.org/ |
| | | | | | | | | | content/10.1101/ |
| | | | | | | | | | 2020.05.12.091298v1) |
| | | | | | | CV2 | 4018 | QQYNNWP | Emilie Seydoux et al., |
| | | | | | | | | PSLT | 2020 |
| | | | | | | | | | (https://www.biorxiv.org/ |
| | | | | | | | | | content/10.1101/ |
| | | | | | | | | | 2020.05.12.091298v1) |
| | | | | | | CV21 | 4019 | QQRSNWP | Emilie Seydoux et al., |
| | | | | | | | | LT | 2020 |
| | | | | | | | | | (https://www.biorxiv.org/ |
| | | | | | | | | | content/10.1101/ |
| | | | | | | | | | 2020.05.12.091298v1) |
| | | | | | | CV22 | 4020 | QTWGTGI | Emilie Seydoux et al., |
| | | | | | | | | RV | 2020 |
| | | | | | | | | | (https://www.biorxiv.org/ |
| | | | | | | | | | content/10.1101/ |
| | | | | | | | | | 2020.05.12.091298v1) |
| | | | | | | CV23 | 4021 | QSADSSGT | Emilie Seydoux et al., |
| | | | | | | | | YVV | 2020 |
| | | | | | | | | | (https://www.biorxiv.org/ |
| | | | | | | | | | content/10.1101/ |
| | | | | | | | | | 2020.05.12.091298v1) |
| | | | | | | CV24 | 4022 | GTWDSSLS | Emilie Seydoux et al., |
| | | | | | | | | ASYV | 2020 |
| | | | | | | | | | (https://www.biorxiv.org/ |
| | | | | | | | | | content/10.1101/ |
| | | | | | | | | | 2020.05.12.091298v1) |
| | | | | | | CV25 | 4023 | QQYNNWP | Emilie Seydoux et al., |
| | | | | | | | | | 2020 |
| | | | | | | | | | (https://www.biorxiv.org/ |
| | | | | | | | | | content/10.1101/ |
| | | | | | | | | | 2020.05.12.091298v1) |
| | | | | | | CV26 | 4024 | LQHNSYPF | Emilie Seydoux et al., |
| | | | | | | | | T | 2020 |
| | | | | | | | | | (https://www.biorxiv.org/ |
| | | | | | | | | | content/10.1101/ |
| | | | | | | | | | 2020.05.12.091298v1) |
| | | | | | | CV27 | 4025 | SSYTSSSTP | Emilie Seydoux et al., |
| | | | | | | | | YV | 2020 |
| | | | | | | | | | (https://www.biorxiv.org/ |
| | | | | | | | | | content/10.1101/ |
| | | | | | | | | | 2020.05.12.091298v1) |
| | | | | | | CV3 | 4026 | QQSYSNPL | Emilie Seydoux et al., |
| | | | | | | | | T | 2020 |
| | | | | | | | | | (https://www.biorxiv.org/ |
| | | | | | | | | | content/10.1101/ |
| | | | | | | | | | 2020.05.12.091298v1) |
| | | | | | | CV30 | 4027 | QQYGSSP | Emilie Seydoux et al., |
| | | | | | | | | QT | 2020 |
| | | | | | | | | | (https://www.biorxiv.org/ |
| | | | | | | | | | content/10.1101/ |
| | | | | | | | | | 2020.05.12.091298v1); |
| | | | | | | | | | Nicholas Hurlburt et al., |
| | | | | | | | | | 2020 |
| | | | | | | | | | (https://www.biorxiv.or |
| | | | | | | | | | g/content/10.1101/202 |
| | | | | | | | | | 0.06.12.148692V1) |
| | | | | | | CV31 | 4028 | GTWDSSLS | Emilie Seydoux et al., |
| | | | | | | | | ASYV | 2020 |
| | | | | | | | | | (https://www.biorxiv.org/ |
| | | | | | | | | | content/10.1101/ |
| | | | | | | | | | 2020.05.12.091298v1) |
| | | | | | | CV32 | 4029 | GTWDSSLS | Emilie Seydoux et al., |
| | | | | | | | | AW | 2020 |
| | | | | | | | | | (https://www.biorxiv.org/ |
| | | | | | | | | | content/10.1101/ |
| | | | | | | | | | 2020.05.12.091298v1) |
| | | | | | | CV33 | 4030 | QSYDSSLS | Emilie Seydoux et al., |
| | | | | | | | | GPVV | 2020 |
| | | | | | | | | | (https://www.biorxiv.org/ |
| | | | | | | | | | content/10.1101/ |
| | | | | | | | | | 2020.05.12.091298v1) |
| | | | | | | CV34 | 4031 | QVWDSSS | Emilie Seydoux et al., |
| | | | | | | | | DHVV | 2020 |
| | | | | | | | | | (https://www.biorxiv.org/ |
| | | | | | | | | | content/10.1101/ |
| | | | | | | | | | 2020.05.12.091298v1) |
| | | | | | | CV35 | 4032 | AAWDDSL | Emilie Seydoux et al., |
| | | | | | | | | NGPV | 2020 |
| | | | | | | | | | (https://www.biorxiv.org/ |
| | | | | | | | | | content/10.1101/ |
| | | | | | | | | | 2020.05.12.091298v1) |
| | | | | | | CV36 | 4033 | QSADSSGT | Emilie Seydoux et al., |
| | | | | | | | | YMI | 2020 |
| | | | | | | | | | (https://www.biorxiv.org/ |
| | | | | | | | | | content/10.1101/ |
| | | | | | | | | | 2020.05.12.091298v1) |
| | | | | | | CV37 | 4034 | QQYDNLP | Emilie Seydoux et al., |
| | | | | | | | | R | 2020 |
| | | | | | | | | | (https://www.biorxiv.org/ |
| | | | | | | | | | content/10.1101/ |
| | | | | | | | | | 2020.05.12.091298v1) |
| | | | | | | CV38 | 4035 | QQRSNWP | Emilie Seydoux et al., |
| | | | | | | | | PIT | 2020 |
| | | | | | | | | | (https://www.biorxiv.org/ |
| | | | | | | | | | content/10.1101/ |
| | | | | | | | | | 2020.05.12.091298v1) |
| | | | | | | CV38-113 | 4036 | QQYDNLPS | Jakob Kreye et al., 2020 |
| | | | | | | | | WT | (https://www.biorxiv.org/ |
| | | | | | | | | | content/10.1101/ |
| | | | | | | | | | 2020.08.15.252320v1.full. |
| | | | | | | | | | pdf) |
| | | | | | | CV38-139 | 4037 | QQLNSYPP | Jakob Kreye et al., 2020 |
| | | | | | | | | GT | (https://www.biorxiv.org/ |
| | | | | | | | | | content/10.1101/ |
| | | | | | | | | | 2020.08.15.252320v1.full. |
| | | | | | | | | | pdf) |
| | | | | | | CV38-142 | 4038 | QQSYSTPR | Jakob Kreye et al., 2020 |
| | | | | | | | | QWT | (https://www.biorxiv.org/ |
| | | | | | | | | | content/10.1101/ |
| | | | | | | | | | 2020.08.15.252320v1.full. |
| | | | | | | | | | pdf) |
| | | | | | | CV38-183 | 4039 | QSYDSSLS | Jakob Kreye et al., 2020 |
| | | | | | | | | GSV | (https://www.biorxiv.org/ |
| | | | | | | | | | content/10.1101/ |
| | | | | | | | | | 2020.08.15.252320v1.full. |
| | | | | | | | | | pdf) |
| | | | | | | CV38-221 | 4040 | QQLYT | Jakob Kreye et al., 2020 |
| | | | | | | | | | (https://www.biorxiv.org/ |
| | | | | | | | | | content/10.1101/ |
| | | | | | | | | | 2020.08.15.252320v1.full. |
| | | | | | | | | | pdf) |
| | | | | | | CV39 | 4041 | MQGTHW | Emilie Seydoux et al., |
| | | | | | | | | PVT | 2020 |
| | | | | | | | | | (https://www.biorxiv.org/ |
| | | | | | | | | | content/10.1101/ |
| | | | | | | | | | 2020.05.12.091298v1) |
| | | | | | | CV4 | 4042 | QQYNSYT | Emilie Seydoux et al., |
| | | | | | | | | | 2020 |
| | | | | | | | | | (https://www.biorxiv.org/ |
| | | | | | | | | | content/10.1101/ |
| | | | | | | | | | 2020.05.12.091298v1) |
| | | | | | | CV40 | 4043 | LQHNSYPL | Emilie Seydoux et al., |
| | | | | | | | | T | 2020 |
| | | | | | | | | | (https://www.biorxiv.org/ |
| | | | | | | | | | content/10.1101/ |
| | | | | | | | | | 2020.05.12.091298v1) |
| | | | | | | CV41 | 4044 | QQYNNWP | Emilie Seydoux et al., |
| | | | | | | | | LT | 2020 |
| | | | | | | | | | (https://www.biorxiv.org/ |
| | | | | | | | | | content/10.1101/ |
| | | | | | | | | | 2020.05.12.091298v1) |
| | | | | | | CV42 | 4045 | QQTYITAF | Emilie Seydoux et al., |
| | | | | | | | | T | 2020 |
| | | | | | | | | | (https://www.biorxiv.org/ |
| | | | | | | | | | content/10.1101/ |
| | | | | | | | | | 2020.05.12.091298v1) |
| | | | | | | CV43 | 4046 | QSYDSSN | Emilie Seydoux et al., |
| | | | | | | | | WV | 2020 |
| | | | | | | | | | (https://www.biorxiv.org/ |
| | | | | | | | | | content/10.1101/ |
| | | | | | | | | | 2020.05.12.091298v1) |
| | | | | | | CV44 | 4047 | QSADSSGT | Emilie Seydoux et al., |
| | | | | | | | | YVV | 2020 |
| | | | | | | | | | (https://www.biorxiv.org/ |
| | | | | | | | | | content/10.1101/ |
| | | | | | | | | | 2020.05.12.091298v1) |
| | | | | | | CV45 | 4048 | QSYDSSLTL | Emilie Seydoux et al., |
| | | | | | | | | YV | 2020 |
| | | | | | | | | | (https://www.biorxiv.org/ |
| | | | | | | | | | content/10.1101/ |
| | | | | | | | | | 2020.05.12.091298v1) |
| | | | | | | CV46 | 4049 | MQGTHW | Emilie Seydoux et al., |
| | | | | | | | | PVT | 2020 |
| | | | | | | | | | (https://www.biorxiv.org/ |
| | | | | | | | | | content/10.1101/ |
| | | | | | | | | | 2020.05.12.091298v1) |
| | | | | | | CV47 | 4050 | LQHNSYPL | Emilie Seydoux et al., |
| | | | | | | | | T | 2020 |
| | | | | | | | | | (https://www.biorxiv.org/ |
| | | | | | | | | | content/10.1101/ |
| | | | | | | | | | 2020.05.12.091298v1) |
| | | | | | | CV48 | 4051 | MQGTHW | Emilie Seydoux et al., |
| | | | | | | | | PPT | 2020 |
| | | | | | | | | | (https://www.biorxiv.org/ |
| | | | | | | | | | content/10.1101/ |
| | | | | | | | | | 2020.05.12.091298v1) |
| | | | | | | CV5 | 4052 | QQYYITPYT | Emilie Seydoux et al., |
| | | | | | | | | | 2020 |
| | | | | | | | | | (https://www.biorxiv.org/ |
| | | | | | | | | | content/10.1101/ |
| | | | | | | | | | 2020.05.12.091298v1) |
| | | | | | | CV50 | 4053 | YSTDSSGN | Emilie Seydoux et al., |
| | | | | | | | | LYV | 2020 |
| | | | | | | | | | (https://www.biorxiv.org/ |
| | | | | | | | | | content/10.1101/ |
| | | | | | | | | | 2020.05.12.091298v1) |
| | | | | | | CV7 | 4054 | QQYNSYT | Emilie Seydoux et al., |
| | | | | | | | | | 2020 |
| | | | | | | | | | (https://www.biorxiv.org/ |
| | | | | | | | | | content/10.1101/ |
| | | | | | | | | | 2020.05.12.091298v1) |
| | | | | | | CV8 | 4055 | QQYGSSP | Emilie Seydoux et al., |
| | | | | | | | | GT | 2020 |
| | | | | | | | | | (https://www.biorxiv.org/ |
| | | | | | | | | | content/10.1101/ |
| | | | | | | | | | 2020.05.12.091298v1) |
| | | | | | | CV9 | 4056 | SSYTSISTW | Emilie Seydoux et al., |
| | | | | | | | | V | 2020 |
| | | | | | | | | | (https://www.biorxiv.org/ |
| | | | | | | | | | content/10.1101/ |
| | | | | | | | | | 2020.05.12.091298v1) |
| | | | | | | EY6A | 4057 | QQSYSTLA | Darning Zhou et al., |
| | | | | | | | | LT | 2020 |
| | | | | | | | | | (https://www.biorxiv.org/ |
| | | | | | | | | | content/10.1101/ |
| | | | | | | | | | 2020.06.12.148387V1) |
| | | | | | | Fab 2-4 | 4058 | SSYAGSNN | David Ho et al., 2020 |
| | | | | | | | | LV | (https://www.biorxiv.org/ |
| | | | | | | | | | content/10.1101/ |
| | | | | | | | | | 2020.06.17.153486V1) |
| | | | | | | FnC1t1p2_ | 4059 | QQYGSSP | Christoph Kreer et al., |
| | | | | | | A5 | | GT | 2020 |
| | | | | | | | | | (https://doi.org/10.1016/ |
| | | | | | | | | | j.cell.2020.06.044) |
| | | | | | | FnC1t2p1_ | 4060 | QQYDNLPL | Christoph Kreer et al., |
| | | | | | | D4 | | T | 2020 |
| | | | | | | | | | (https://doi.org/10.1016/ |
| | | | | | | | | | j.cell.2020.06.044) |
| | | | | | | FnC1t2p1_ | 4061 | QQYDNLPL | Christoph Kreer et al., |
| | | | | | | G5 | | T | 2020 |
| | | | | | | | | | (https://doi.org/10.1016/ |
| | | | | | | | | | j.cell.2020.06.044) |
| | | | | | | H014 | | ND | Zhe Lv et al., 2020 |
| | | | | | | | | | (https://www.biorxiv.org/ |
| | | | | | | | | | content/10.1101/ |
| | | | | | | | | | 2020.06.02.129098v2) |
| | | | | | | H11-D4 | | N/A | Jiangdong Huo et al., |
| | | | | | | | | | 2020 |
| | | | | | | | | | (https://www.nature.com/ |
| | | | | | | | | | articles/s41594-020- |
| | | | | | | | | | 0469-6) |
| | | | | | | H11-H4 | | N/A | Jiangdong Huo et al., |
| | | | | | | | | | 2020 |
| | | | | | | | | | (https://www.nature.com/ |
| | | | | | | | | | articles/s41594-020- |
| | | | | | | | | | 0469-6) |
| | | | | | | H4 | 4062 | MQRIEFPL | Yan Wu et al., 2020 |
| | | | | | | | | T | (https://science.sciencemag. |
| | | | | | | | | | org/content/early/ |
| | | | | | | | | | 2020/05/12/science.abc2241) |
| | | | | | | HbnC2t1p2_ | 4063 | QQRSNWP | Christoph Kreer et al., |
| | | | | | | D9 | | PTWT | 2020 |
| | | | | | | | | | (https://doi.org/10.1016/ |
| | | | | | | | | | j.cell.2020.06.044) |
| | | | | | | HbnC3t1p1_ | 4064 | QQYGSSP | Christoph Kreer et al., |
| | | | | | | C6 | | WT | 2020 |
| | | | | | | | | | (https://doi.org/10.1016/ |
| | | | | | | | | | j.cell.2020.06.044) |
| | | | | | | HbnC3t1p1_ | 4065 | QHYHSFPL | Christoph Kreer et al., |
| | | | | | | F4 | | T | 2020 |
| | | | | | | | | | (https://doi.org/10.1016/ |
| | | | | | | | | | j.cell.2020.06.044) |
| | | | | | | HbnC3t1p1_ | 4066 | QQYGSSPR | Christoph Kreer et al., |
| | | | | | | G4 | | T | 2020 |
| | | | | | | | | | (https://doi.org/10.1016/ |
| | | | | | | | | | j.cell.2020.06.044) |
| | | | | | | HbnC3t1p2_ | 4067 | QQYGSSPR | Christoph Kreer et al., |
| | | | | | | B10 | | T | 2020 |
| | | | | | | | | | (https://doi.org/10.1016/ |
| | | | | | | | | | j.cell.2020.06.044) |
| | | | | | | HbnC3t1p2_ | 4068 | QQYGRSP | Christoph Kreer et al., |
| | | | | | | C6 | | WT | 2020 |
| | | | | | | | | | (https://doi.org/10.1016/ |
| | | | | | | | | | j.cell.2020.06.044) |
| | | | | | | HbnC4t1p1_ | 4069 | QQSYSNPL | Christoph Kreer et al., |
| | | | | | | D5 | | T | 2020 |
| | | | | | | | | | (https://doi.org/10.1016/ |
| | | | | | | | | | j.cell.2020.06.044) |
| | | | | | | Ju et al., | | Various | Bin Ju et al., 2020 |
| | | | | | | 2020 | | | (https://www.nature.com/ |
| | | | | | | | | | articles/s41586-020- |
| | | | | | | | | | 2380-z) |
| | | | | | | Kim et | | Various | Sang II Kim et al., 2020 |
| | | | | | | al., 2020 | | | (https://www.biorxiv.org/ |
| | | | | | | | | | content/10.1101/ |
| | | | | | | | | | 2020.06.26.174557v2) |
| | | | | | | LR1 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | LR11 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | LR15 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | LR16 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | LR2 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | LR3 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | LR5 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | LR6 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | LR7 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | LR8 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | mAb-1 | 4070 | QSYDSSLS | Anna Wee et al., 2020 |
| | | | | | | | | VLYV | (https://science.sciencemag. |
| | | | | | | | | | org/content/early/ |
| | | | | | | | | | 2020/06/15/science.abc7424) |
| | | | | | | mAb-10 | 4071 | QQYNSWP | Anna Wee et al., 2020 |
| | | | | | | | | PLT | (https://science.sciencemag. |
| | | | | | | | | | org/content/early/ |
| | | | | | | | | | 2020/06/15/science.abc7424) |
| | | | | | | mAb-100 | 4072 | QRRGDGY | Anna Wee et al., 2020 |
| | | | | | | | | N | (https://science.sciencemag. |
| | | | | | | | | | org/content/early/ |
| | | | | | | | | | 2020/06/15/science.abc7424) |
| | | | | | | mAb-101 | 4073 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-102 | 4074 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-103 | 4075 | QQSYSTPY | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-104 | 4076 | CSYAGSGT | Anna Wee et al., 2020 |
| | | | | | | | | WI | (https://science.sciencemag. |
| | | | | | | | | | org/content/early/ |
| | | | | | | | | | 2020/06/15/science.abc7424) |
| | | | | | | mAb-105 | 4077 | HQYGSSP | Anna Wee et al., 2020 |
| | | | | | | | | WT | (https://science.sciencemag. |
| | | | | | | | | | org/content/early/ |
| | | | | | | | | | 2020/06/15/science.abc7424) |
| | | | | | | mAb-106 | 4078 | QRRGDGY | Anna Wee et al., 2020 |
| | | | | | | | | N | (https://science.sciencemag. |
| | | | | | | | | | org/content/early/ |
| | | | | | | | | | 2020/06/15/science.abc7424) |
| | | | | | | mAb-107 | 4079 | MQATDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-108 | 4080 | MQTTDWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-109 | 4081 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-11 | 4082 | QQFEDLPI | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-110 | 4083 | QQYYSTPY | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-111 | 4084 | QQYGTSPV | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-112 | 4085 | GTWDFSLS | Anna Wee et al., 2020 |
| | | | | | | | | AGV | (https://science.sciencemag. |
| | | | | | | | | | org/content/early/ |
| | | | | | | | | | 2020/06/15/science.abc7424) |
| | | | | | | mAb-113 | 4086 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-114 | 4087 | QQYKSPLS | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-115 | 4088 | QQRSDWH | Anna Wee et al., 2020 |
| | | | | | | | | PIT | (https://science.sciencemag. |
| | | | | | | | | | org/content/early/ |
| | | | | | | | | | 2020/06/15/science.abc7424) |
| | | | | | | mAb-116 | 4089 | QQSYSTPG | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-117 | 4090 | QQRAKWP | Anna Wee et al., 2020 |
| | | | | | | | | PRVT | (https://science.sciencemag. |
| | | | | | | | | | org/content/early/ |
| | | | | | | | | | 2020/06/15/science.abc7424) |
| | | | | | | mAb-118 | 4091 | QQRAKWP | Anna Wee et al., 2020 |
| | | | | | | | | PRVI | (https://science.sciencemag. |
| | | | | | | | | | org/content/early/ |
| | | | | | | | | | 2020/06/15/science.abc7424) |
| | | | | | | mAb-119 | 4092 | YSTDSTAN | Anna Wee et al., 2020 |
| | | | | | | | | YKV | (https://science.sciencemag. |
| | | | | | | | | | org/content/early/ |
| | | | | | | | | | 2020/06/15/science.abc7424) |
| | | | | | | mAb-12 | 4093 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-120 | 4094 | CSYAGSSA | Anna Wee et al., 2020 |
| | | | | | | | | VVVV | (https://science.sciencemag. |
| | | | | | | | | | org/content/early/ |
| | | | | | | | | | 2020/06/15/science.abc7424) |
| | | | | | | mAb-121 | 4095 | QQYYSTPY | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-122 | 4096 | LQRSDWH | Anna Wee et al., 2020 |
| | | | | | | | | PIT | (https://science.sciencemag. |
| | | | | | | | | | org/content/early/ |
| | | | | | | | | | 2020/06/15/science.abc7424) |
| | | | | | | mAb-123 | 4097 | QQRSNWP | Anna Wee et al., 2020 |
| | | | | | | | | PRLT | (https://science.sciencemag. |
| | | | | | | | | | org/content/early/ |
| | | | | | | | | | 2020/06/15/science.abc7424) |
| | | | | | | mAb-124 | 4098 | LQYSLATT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-125 | 4099 | ETWDSNL | Anna Wee et al., 2020 |
| | | | | | | | | KGV | (https://science.sciencemag. |
| | | | | | | | | | org/content/early/ |
| | | | | | | | | | 2020/06/15/science.abc7424) |
| | | | | | | mAb-126 | 4100 | EAWDNNN | Anna Wee et al., 2020 |
| | | | | | | | | LGV | (https://science.sciencemag. |
| | | | | | | | | | org/content/early/ |
| | | | | | | | | | 2020/06/15/science.abc7424) |
| | | | | | | mAb-127 | 4101 | LQHNSYPL | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-128 | 4102 | MQALQTP | Anna Wee et al., 2020 |
| | | | | | | | | GVT | (https://science.sciencemag. |
| | | | | | | | | | org/content/early/ |
| | | | | | | | | | 2020/06/15/science.abc7424) |
| | | | | | | mAb-129 | 4103 | ATWDDSL | Anna Wee et al., 2020 |
| | | | | | | | | NGVV | (https://science.sciencemag. |
| | | | | | | | | | org/content/early/ |
| | | | | | | | | | 2020/06/15/science.abc7424) |
| | | | | | | mAb-13 | 4104 | MQGTDW | Anna Wee et al., 2020 |
| | | | | | | | | PRS | (https://science.sciencemag. |
| | | | | | | | | | org/content/early/ |
| | | | | | | | | | 2020/06/15/science.abc7424) |
| | | | | | | mAb-130 | 4105 | LHTYTTPRT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-131 | 4106 | QQYNYWP | Anna Wee et al., 2020 |
| | | | | | | | | PLT | (https://science.sciencemag. |
| | | | | | | | | | org/content/early/ |
| | | | | | | | | | 2020/06/15/science.abc7424) |
| | | | | | | mAb-132 | 4107 | QQSYRFPI | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-133 | 4108 | QQRTNWP | Anna Wee et al., 2020 |
| | | | | | | | | GAT | (https://science.sciencemag. |
| | | | | | | | | | org/content/early/ |
| | | | | | | | | | 2020/06/15/science.abc7424) |
| | | | | | | mAb-134 | 4109 | HTWGTDI | Anna Wee et al., 2020 |
| | | | | | | | | QV | (https://science. science |
| | | | | | | | | | mag.org/content/early/ |
| | | | | | | | | | 2020/06/15/science.abc |
| | | | | | | | | | 7424) |
| | | | | | | mAb-135 | 4110 | QSYDSSSQ | Anna Wee et al., 2020 |
| | | | | | | | | V | (https://science.sciencemag. |
| | | | | | | | | | org/content/early/ |
| | | | | | | | | | 2020/06/15/science.abc7424) |
| | | | | | | mAb-136 | 4111 | QQYNYWP | Anna Wee et al., 2020 |
| | | | | | | | | PLT | (https://science.sciencemag. |
| | | | | | | | | | org/content/early/ |
| | | | | | | | | | 2020/06/15/science.abc7424) |
| | | | | | | mAb-137 | 4112 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-138 | 4113 | QQYNSLYT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-139 | 4114 | QQYHHWP | Anna Wee et al., 2020 |
| | | | | | | | | PYT | (https://science.sciencemag. |
| | | | | | | | | | org/content/early/ |
| | | | | | | | | | 2020/06/15/science.abc7424) |
| | | | | | | mAb-14 | 4115 | QQYCSTPP | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-140 | 4116 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-141 | 4117 | MQGTDW | Anna Wee et al., 2020 |
| | | | | | | | | PRS | (https://science.sciencemag. |
| | | | | | | | | | org/content/early/ |
| | | | | | | | | | 2020/06/15/science.abc7424) |
| | | | | | | mAb-142 | 4118 | QQSYSTPLI | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-143 | 4119 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-144 | 4120 | MQGTDW | Anna Wee et al., 2020 |
| | | | | | | | | PRT | (https://science. science |
| | | | | | | | | | mag.org/content/early/ |
| | | | | | | | | | 2020/06/15/science.abc |
| | | | | | | | | | 7424) |
| | | | | | | mAb-145 | 4121 | QQRFSWY | Anna Wee et al., 2020 |
| | | | | | | | | N | (https://science.sciencemag. |
| | | | | | | | | | org/content/early/ |
| | | | | | | | | | 2020/06/15/science.abc7424) |
| | | | | | | mAb-146 | 4122 | CSYGGRST | Anna Wee et al., 2020 |
| | | | | | | | | SVV | (https://science.sciencemag. |
| | | | | | | | | | org/content/early/ |
| | | | | | | | | | 2020/06/15/science.abc7424) |
| | | | | | | mAb-147 | 4123 | QQRFSWY | Anna Wee et al., 2020 |
| | | | | | | | | N | (https://science.sciencemag. |
| | | | | | | | | | org/content/early/ |
| | | | | | | | | | 2020/06/15/science.abc7424) |
| | | | | | | mAb-148 | 4124 | SSYTSGGTL | Anna Wee et al., 2020 |
| | | | | | | | | V | (https://science.sciencemag. |
| | | | | | | | | | org/content/early/ |
| | | | | | | | | | 2020/06/15/science.abc7424) |
| | | | | | | mAb-149 | 4125 | MQATEWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-15 | 4126 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-150 | 4127 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-151 | 4128 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-152 | 4129 | MQGTEW | Anna Wee et al., 2020 |
| | | | | | | | | PRT | (https://science. science |
| | | | | | | | | | mag.org/content/early/ |
| | | | | | | | | | 2020/06/15/science.abc |
| | | | | | | | | | 7424) |
| | | | | | | mAb-153 | 4130 | MQATEWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-154 | 4131 | QSADSNDS | Anna Wee et al., 2020 |
| | | | | | | | | SPV | (https://science.sciencemag. |
| | | | | | | | | | org/content/early/ |
| | | | | | | | | | 2020/06/15/science.abc7424) |
| | | | | | | mAb-155 | 4132 | HQRSNWP | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-156 | 4133 | QQSYSIPW | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-157 | 4134 | QHSYSSPP | Anna Wee et al., 2020 |
| | | | | | | | | LT | (https://science.sciencemag. |
| | | | | | | | | | org/content/early/ |
| | | | | | | | | | 2020/06/15/science.abc7424) |
| | | | | | | mAb-158 | 4135 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-159 | 4136 | MQTTDWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-16 | 4137 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-160 | 4138 | HQYSGSAT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-161 | 4139 | ATWDDILN | Anna Wee et al., 2020 |
| | | | | | | | | GPV | (https://science.sciencemag. |
| | | | | | | | | | org/content/early/ |
| | | | | | | | | | 2020/06/15/science.abc7424) |
| | | | | | | mAb-162 | 4140 | QKYNRAP | Anna Wee et al., 2020 |
| | | | | | | | | WT | (https://science.sciencemag. |
| | | | | | | | | | org/content/early/ |
| | | | | | | | | | 2020/06/15/science.abc7424) |
| | | | | | | mAb-163 | 4141 | SSYISDIKLV | Anna Wee et al., 2020 |
| | | | | | | | | V | (https://science.sciencemag. |
| | | | | | | | | | org/content/early/ |
| | | | | | | | | | 2020/06/15/science.abc7424) |
| | | | | | | mAb-164 | 4142 | CSYAGTYI | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-165 | 4143 | CSYRSDNT | Anna Wee et al., 2020 |
| | | | | | | | | YI | (https://science.sciencemag. |
| | | | | | | | | | org/content/early/ |
| | | | | | | | | | 2020/06/15/science.abc7424) |
| | | | | | | mAb-166 | 4144 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-167 | 4145 | AAWDDSL | Anna Wee et al., 2020 |
| | | | | | | | | NTFRYV | (https://science.sciencemag. |
| | | | | | | | | | org/content/early/ |
| | | | | | | | | | 2020/06/15/science.abc7424) |
| | | | | | | mAb-168 | 4146 | QQSYNTFF | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-169 | 4147 | QHYDSYPT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-17 | 4148 | MQGTEWL | Anna Wee et al., 2020 |
| | | | | | | | | GT | (https://science.sciencemag. |
| | | | | | | | | | org/content/early/ |
| | | | | | | | | | 2020/06/15/science.abc7424) |
| | | | | | | mAb-170 | 4149 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-171 | 4150 | MQTTDWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-172 | 4151 | QQSYSTPP | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-173 | 4152 | HQYNKWP | Anna Wee et al., 2020 |
| | | | | | | | | PIT | (https://science.sciencemag. |
| | | | | | | | | | org/content/early/ |
| | | | | | | | | | 2020/06/15/science.abc7424) |
| | | | | | | mAb-174 | 4153 | KQYNSYPY | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-175 | 4154 | QQSYSDS | Anna Wee et al., 2020 |
| | | | | | | | | WT | (https://science.sciencemag. |
| | | | | | | | | | org/content/early/ |
| | | | | | | | | | 2020/06/15/science.abc7424) |
| | | | | | | mAb-176 | 4155 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-177 | 4156 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-178 | 4157 | QQYYTTPY | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-179 | 4158 | QQTYSTPP | Anna Wee et al., 2020 |
| | | | | | | | | EGPT | (https://science.sciencemag. |
| | | | | | | | | | org/content/early/ |
| | | | | | | | | | 2020/06/15/science.abc7424) |
| | | | | | | mAb-18 | 4159 | CSYAGASP | Anna Wee et al., 2020 |
| | | | | | | | | FVV | (https://science.sciencemag. |
| | | | | | | | | | org/content/early/ |
| | | | | | | | | | 2020/06/15/science.abc7424) |
| | | | | | | mAb-180 | 4160 | QQYGSSYT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-181 | 4161 | MQATEWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-182 | 4162 | MQTTDWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-183 | 4163 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-184 | 4164 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-185 | 4165 | CSYAGDDT | Anna Wee et al., 2020 |
| | | | | | | | | W | (https://science.sciencemag. |
| | | | | | | | | | org/content/early/ |
| | | | | | | | | | 2020/06/15/science.abc7424) |
| | | | | | | mAb-186 | 4166 | HQYGGSPT | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-187 | 4167 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-188 | 4168 | QQRTSTLT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-189 | 4169 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-19 | 4170 | ETWDSSLS | Anna Wee et al., 2020 |
| | | | | | | | | VW | (https://science.sciencemag. |
| | | | | | | | | | org/content/early/ |
| | | | | | | | | | 2020/06/15/science.abc7424) |
| | | | | | | mAb-190 | 4171 | GTWDSRLS | Anna Wee et al., 2020 |
| | | | | | | | | AW | (https://science.sciencemag. |
| | | | | | | | | | org/content/early/ |
| | | | | | | | | | 2020/06/15/science.abc7424) |
| | | | | | | mAb-191 | 4172 | QQRSNWP | Anna Wee et al., 2020 |
| | | | | | | | | QN | (https://science.sciencemag. |
| | | | | | | | | | org/content/early/ |
| | | | | | | | | | 2020/06/15/science.abc7424) |
| | | | | | | mAb-192 | 4173 | QQYDSYPV | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-193 | 4174 | QQYYTMW | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-194 | 4175 | GTWDSSLS | Anna Wee et al., 2020 |
| | | | | | | | | VDNYV | (https://science.sciencemag. |
| | | | | | | | | | org/content/early/ |
| | | | | | | | | | 2020/06/15/science.abc7424) |
| | | | | | | mAb-195 | 4176 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-196 | 4177 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-197 | 4178 | QQSYSTPL | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-2 | 4179 | QQRSNWP | Anna Wee et al., 2020 |
| | | | | | | | | QN | (https://science.sciencemag. |
| | | | | | | | | | org/content/early/ |
| | | | | | | | | | 2020/06/15/science.abc7424) |
| | | | | | | mAb-20 | 4180 | QSYDSSLS | Anna Wee et al., 2020 |
| | | | | | | | | AS | (https://science.sciencemag. |
| | | | | | | | | | org/content/early/ |
| | | | | | | | | | 2020/06/15/science.abc7424) |
| | | | | | | mAb-21 | 4181 | QSYDSSLS | Anna Wee et al., 2020 |
| | | | | | | | | GVL | (https://science.sciencemag. |
| | | | | | | | | | org/content/early/ |
| | | | | | | | | | 2020/06/15/science.abc7424) |
| | | | | | | mAb-22 | 4182 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-23 | 4183 | QQYNTWP | Anna Wee et al., 2020 |
| | | | | | | | | PLT | (https://science.sciencemag. |
| | | | | | | | | | org/content/early/ |
| | | | | | | | | | 2020/06/15/science.abc7424) |
| | | | | | | mAb-24 | 4184 | MQGTHW | Anna Wee et al., 2020 |
| | | | | | | | | PRS | (https://science.sciencemag. |
| | | | | | | | | | org/content/early/ |
| | | | | | | | | | 2020/06/15/science.abc7424) |
| | | | | | | mAb-25 | 4185 | QQYGSAPL | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-26 | 4186 | QQYGNSP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-27 | 4187 | ETWDTSLS | Anna Wee et al., 2020 |
| | | | | | | | | VW | (https://science.sciencemag. |
| | | | | | | | | | org/content/early/ |
| | | | | | | | | | 2020/06/15/science.abc7424) |
| | | | | | | mAb-28 | 4188 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-29 | 4189 | LQYSDATT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-3 | 4190 | QQSYTTPIT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-30 | 4191 | MQATEWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-31 | 4192 | QQRRNWP | Anna Wee et al., 2020 |
| | | | | | | | | PMYT | (https://science.sciencemag. |
| | | | | | | | | | org/content/early/ |
| | | | | | | | | | 2020/06/15/science.abc7424) |
| | | | | | | mAb-32 | 4193 | MQTTDWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-33 | 4194 | MQTTDWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-34 | 4195 | QSYDSGNL | Anna Wee et al., 2020 |
| | | | | | | | | W | (https://science.sciencemag. |
| | | | | | | | | | org/content/early/ |
| | | | | | | | | | 2020/06/15/science.abc7424) |
| | | | | | | mAb-35 | 4196 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-36 | 4197 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-37 | 4198 | QQYGTSPV | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-38 | 4199 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-39 | 4200 | QQRRNWP | Anna Wee et al., 2020 |
| | | | | | | | | PMYT | (https://science.sciencemag. |
| | | | | | | | | | org/content/early/ |
| | | | | | | | | | 2020/06/15/science.abc7424) |
| | | | | | | mAb-4 | 4201 | QSYDSSLS | Anna Wee et al., 2020 |
| | | | | | | | | VLYV | (https://science.sciencemag. |
| | | | | | | | | | org/content/early/ |
| | | | | | | | | | 2020/06/15/science.abc7424) |
| | | | | | | mAb-40 | 4202 | QHRTNWP | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-41 | 4203 | FLSYRGAP | Anna Wee et al., 2020 |
| | | | | | | | | PV | (https://science.sciencemag. |
| | | | | | | | | | org/content/early/ |
| | | | | | | | | | 2020/06/15/science.abc7424) |
| | | | | | | mAb-42 | 4204 | QKHDRDP | Anna Wee et al., 2020 |
| | | | | | | | | WT | (https://science.sciencemag. |
| | | | | | | | | | org/content/early/ |
| | | | | | | | | | 2020/06/15/science.abc7424) |
| | | | | | | mAb-43 | 4205 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-44 | 4206 | QQAESFPF | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-45 | 4207 | MQATDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-46 | 4208 | QQRGNGY | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-47 | 4209 | MQATEWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-48 | 4210 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-49 | 4211 | MQATDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-5 | 4212 | QQSYSVPL | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-50 | 4213 | MQATDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-51 | 4214 | STYTSTSTI | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-52 | 4215 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-53 | 4216 | MQATDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-54 | 4217 | QQTYITPG | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-55 | 4218 | QQYYLTP | Anna Wee et al., 2020 |
| | | | | | | | | WT | (https://science.sciencemag. |
| | | | | | | | | | org/content/early/ |
| | | | | | | | | | 2020/06/15/science.abc7424) |
| | | | | | | mAb-56 | 4219 | CSYAVSGT | Anna Wee et al., 2020 |
| | | | | | | | | VL | (https://science.sciencemag. |
| | | | | | | | | | org/content/early/ |
| | | | | | | | | | 2020/06/15/science.abc7424) |
| | | | | | | mAb-57 | 4220 | LQYSLATT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-58 | 4221 | QQRSDGY | Anna Wee et al., 2020 |
| | | | | | | | | N | (https://science.sciencemag. |
| | | | | | | | | | org/content/early/ |
| | | | | | | | | | 2020/06/15/science.abc7424) |
| | | | | | | mAb-59 | 4222 | QQTYITPT | Anna Wee et al., 2020 |
| | | | | | | | | WT | (https://science.sciencemag. |
| | | | | | | | | | org/content/early/ |
| | | | | | | | | | 2020/06/15/science.abc7424) |
| | | | | | | mAb-6 | 4223 | QQSYSTPY | Anna Wee et al., 2020 |
| | | | | | | | | N | (https://science.sciencemag. |
| | | | | | | | | | org/content/early/ |
| | | | | | | | | | 2020/06/15/science.abc7424) |
| | | | | | | mAb-60 | 4224 | QQYNSOY | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-61 | 4225 | CSSPGTIT | Anna Wee et al., 2020 |
| | | | | | | | | WV | (https://science.sciencemag. |
| | | | | | | | | | org/content/early/ |
| | | | | | | | | | 2020/06/15/science.abc7424) |
| | | | | | | mAb-62 | 4226 | ATWDDILN | Anna Wee et al., 2020 |
| | | | | | | | | GPV | (https://science.sciencemag. |
| | | | | | | | | | org/content/early/ |
| | | | | | | | | | 2020/06/15/science.abc7424) |
| | | | | | | mAb-63 | 4227 | MOATHW | Anna Wee et al., 2020 |
| | | | | | | | | PRA | (https://science.sciencemag. |
| | | | | | | | | | org/content/early/ |
| | | | | | | | | | 2020/06/15/science.abc7424) |
| | | | | | | mAb-64 | 4228 | HQYGSSP | Anna Wee et al., 2020 |
| | | | | | | | | WT | (https://science.sciencemag. |
| | | | | | | | | | org/content/early/ |
| | | | | | | | | | 2020/06/15/science.abc7424) |
| | | | | | | mAb-65 | 4229 | QHRSNWP | Anna Wee et al., 2020 |
| | | | | | | | | PRYT | (https://science.sciencemag. |
| | | | | | | | | | org/content/early/ |
| | | | | | | | | | 2020/06/15/science.abc7424) |
| | | | | | | mAb-66 | 4230 | YSTDSTAN | Anna Wee et al., 2020 |
| | | | | | | | | YKV | (https://science.sciencemag. |
| | | | | | | | | | org/content/early/ |
| | | | | | | | | | 2020/06/15/science.abc7424) |
| | | | | | | mAb-67 | 4231 | YSTDSTAN | Anna Wee et al., 2020 |
| | | | | | | | | YKV | (https://science. science |
| | | | | | | | | | mag.org/content/early/ |
| | | | | | | | | | 2020/06/15/science.abc |
| | | | | | | | | | 7424) |
| | | | | | | mAb-68 | 4232 | YSTDSTAN | Anna Wee et al., 2020 |
| | | | | | | | | YKV | (https://science.sciencemag. |
| | | | | | | | | | org/content/early/ |
| | | | | | | | | | 2020/06/15/science.abc7424) |
| | | | | | | mAb-69 | 4233 | MIWHDNA | Anna Wee et al., 2020 |
| | | | | | | | | W | (https://science.sciencemag. |
| | | | | | | | | | org/content/early/ |
| | | | | | | | | | 2020/06/15/science.abc7424) |
| | | | | | | mAb-7 | 4234 | HQYNTWP | Anna Wee et al., 2020 |
| | | | | | | | | PLT | (https://science.sciencemag. |
| | | | | | | | | | org/content/early/ |
| | | | | | | | | | 2020/06/15/science.abc7424) |
| | | | | | | mAb-70 | 4235 | MQATHW | Anna Wee et al., 2020 |
| | | | | | | | | PRA | (https://science.sciencemag. |
| | | | | | | | | | org/content/early/ |
| | | | | | | | | | 2020/06/15/science.abc7424) |
| | | | | | | mAb-71 | 4236 | MQSIQVPI | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-72 | 4237 | QQYNLWP | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-73 | 4238 | KQYNSYPY | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-74 | 4239 | ETWDDSLS | Anna Wee et al., 2020 |
| | | | | | | | | AW | (https://science.sciencemag. |
| | | | | | | | | | org/content/early/ |
| | | | | | | | | | 2020/06/15/science.abc7424) |
| | | | | | | mAb-75 | 4240 | MQSIQTPI | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-76 | 4241 | QQYYSTPP | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-77 | 4242 | MQATEWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-78 | 4243 | QQYYTTPL | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-79 | 4244 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-8 | 4245 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | | | | 7424) |
| | | | | | | mAb-80 | 4246 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-81 | 4247 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-82 | 4248 | SSYTSSSTL | Anna Wee et al., 2020 |
| | | | | | | | | YV | (https://science.sciencemag. |
| | | | | | | | | | org/content/early/ |
| | | | | | | | | | 2020/06/15/science.abc7424) |
| | | | | | | mAb-83 | 4249 | QQYNSGW | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-84 | 4250 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-85 | 4251 | LQLHNYSS | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-86 | 4252 | QQRSDGY | Anna Wee et al., 2020 |
| | | | | | | | | N | (https://science.sciencemag. |
| | | | | | | | | | org/content/early/ |
| | | | | | | | | | 2020/06/15/science.abc7424) |
| | | | | | | mAb-87 | 4253 | SSYTSTNTV | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-88 | 4254 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-89 | 4255 | CSSPGTIT | Anna Wee et al., 2020 |
| | | | | | | | | | (https://science.sciencemag. |
| | | | | | | | | | org/content/early/ |
| | | | | | | | | | 2020/06/15/science.abc7424) |
| | | | | | | mAb-9 | 4256 | QQCYSYPP | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-90 | 4257 | QQYYSTPY | Anna Wee et al., 2020 |
| | | | | | | | | T | (https://science.sciencemag. |
| | | | | | | | | | org/content/early/ |
| | | | | | | | | | 2020/06/15/science.abc7424) |
| | | | | | | mAb-91 | 4258 | CSYTRTNT | Anna Wee et al., 2020 |
| | | | | | | | | PVL | (https://science. science |
| | | | | | | | | | mag.org/content/early/ |
| | | | | | | | | | 2020/06/15/science.abc |
| | | | | | | | | | 7424) |
| | | | | | | mAb-92 | 4259 | QQYYSWP | Anna Wee et al., 2020 |
| | | | | | | | | PLT | (https://science.sciencemag. |
| | | | | | | | | | org/content/early/ |
| | | | | | | | | | 2020/06/15/science.abc7424) |
| | | | | | | mAb-93 | 4260 | MQGTDW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-94 | 4261 | MQSTDWP | Anna Wee et al., 2020 |
| | | | | | | | | RT | (https://science.sciencemag. |
| | | | | | | | | | org/content/early/ |
| | | | | | | | | | 2020/06/15/science.abc7424) |
| | | | | | | mAb-95 | 4262 | MQGTEW | Anna Wee et al., 2020 |
| | | | | | | | | PRT | (https://science.sciencemag. |
| | | | | | | | | | org/content/early/ |
| | | | | | | | | | 2020/06/15/science.abc7424) |
| | | | | | | mAb-96 | 4263 | QQYNNWP | Anna Wee et al., 2020 |
| | | | | | | | | SLT | (https://science.sciencemag. |
| | | | | | | | | | org/content/early/ |
| | | | | | | | | | 2020/06/15/science.abc7424) |
| | | | | | | mAb-97 | 4264 | MQATEWP | Anna Wee et al., 2020 |
| | | | | | | | | RT | (https://science.science |
| | | | | | | | | | mag.org/content/early/ |
| | | | | | | | | | 2020/06/15/science.abc7424) |
| | | | | | | mAb-98 | 4265 | QQRSDGY | Anna Wee et al., 2020 |
| | | | | | | | | N | (https://science.sciencemag. |
| | | | | | | | | | org/content/early/ |
| | | | | | | | | | 2020/06/15/science.abc7424) |
| | | | | | | mAb-99 | 4266 | YSTDSTAN | Anna Wee et al., 2020 |
| | | | | | | | | YKV | (https://science.sciencemag. |
| | | | | | | | | | org/content/early/ |
| | | | | | | | | | 2020/06/15/science.abc7424) |
| | | | | | | mBG17 | 4267 | QQFYNYPR | James Terry et al., 2020 |
| | | | | | | | | T | (https://www.biorxiv.org/ |
| | | | | | | | | | content/10.1101/ |
| | | | | | | | | | 2020.09.03.280370v1.full) |
| | | | | | | mBG21 | 4268 | QQDYSSP | James Terry et al., 2020 |
| | | | | | | | | | (https://www.biorxiv.org/ |
| | | | | | | | | | content/10.1101/ |
| | | | | | | | | | 2020.09.03.280370v1.full) |
| | | | | | | mBG22 | 4269 | QQDYSSP | James Terry et al., 2020 |
| | | | | | | | | | (https://www.biorxiv.org/ |
| | | | | | | | | | content/10.1101/ |
| | | | | | | | | | 2020.09.03.280370v1.full) |
| | | | | | | mBG57 | 4270 | MQHLENP | James Terry et al., 2020 |
| | | | | | | | | LGVR | (https://www.biorxiv.org/ |
| | | | | | | | | | content/10.1101/ |
| | | | | | | | | | 2020.09.03.280370v1.full) |
| | | | | | | mBG67 | 4271 | LQYHRSP | James Terry et al., 2020 |
| | | | | | | | | | (https://www.biorxiv.org/ |
| | | | | | | | | | content/10.1101/ |
| | | | | | | | | | 2020.09.03.280370v1.full) |
| | | | | | | MD17 | 4272 | QQSYTTPL | Tal Noy-Porat et al., |
| | | | | | | | | T | 2020 |
| | | | | | | | | | (https://www.biorxiv.org/ |
| | | | | | | | | | content/10.1101/ |
| | | | | | | | | | 2020.05.20.106609v2) |
| | | | | | | MD29 | 4273 | HQTYTSPY | Tal Noy-Porat et al., |
| | | | | | | | | T | 2020 |
| | | | | | | | | | (https://www.biorxiv.org/ |
| | | | | | | | | | content/10.1101/ |
| | | | | | | | | | 2020.05.20.106609v2) |
| | | | | | | MD45 | 4274 | QQYGVSPE | Tal Noy-Porat et al., |
| | | | | | | | | II | 2020 |
| | | | | | | | | | (https://www.biorxiv.org/ |
| | | | | | | | | | content/10.1101/ |
| | | | | | | | | | 2020.05.20.106609v2) |
| | | | | | | MD47 | 4275 | QVWDSSS | Tal Noy-Porat et al., |
| | | | | | | | | HHHW | 2020 |
| | | | | | | | | | (https://www.biorxiv.org/ |
| | | | | | | | | | content/10.1101/ |
| | | | | | | | | | 2020.05.20.106609v2) |
| | | | | | | MD62 | 4276 | QQANSFPL | Tal Noy-Porat et al., |
| | | | | | | | | T | 2020 |
| | | | | | | | | | (https://www.biorxiv.org/ |
| | | | | | | | | | content/10.1101/ |
| | | | | | | | | | 2020.05.20.106609v2) |
| | | | | | | MD63 | 4277 | QQSYSTPY | Tal Noy-Porat et al., |
| | | | | | | | | T | 2020 |
| | | | | | | | | | (https://www.biorxiv.org/ |
| | | | | | | | | | content/10.1101/ |
| | | | | | | | | | 2020.05.20.106609v2) |
| | | | | | | MD65 | 4278 | QQYGSSPL | Tal Noy-Porat et al., |
| | | | | | | | | T | 2020 |
| | | | | | | | | | (https://www.biorxiv.org/ |
| | | | | | | | | | content/10.1101/ |
| | | | | | | | | | 2020.05.20.106609v2) |
| | | | | | | MD67 | 4279 | QQFGSSPL | Tal Noy-Porat et al., |
| | | | | | | | | T | 2020 |
| | | | | | | | | | (https://www.biorxiv.org/ |
| | | | | | | | | | content/10.1101/ |
| | | | | | | | | | 2020.05.20.106609v2) |
| | | | | | | mNb6 | | ND | Michael Schoof et al., |
| | | | | | | | | | 2020 |
| | | | | | | | | | (https://www.biorxiv.org/ |
| | | | | | | | | | content/10.1101/ |
| | | | | | | | | | 2020.08.08.238469v1.full. |
| | | | | | | | | | pdf) |
| | | | | | | mNb6-tri | | ND | Michael Schoof et al., |
| | | | | | | | | | 2020 |
| | | | | | | | | | (https://www.biorxiv.org/ |
| | | | | | | | | | content/10.1101/ |
| | | | | | | | | | 2020.08.08.238469v1.full. |
| | | | | | | | | | pdf) |
| | | | | | | MnCit3p1_ | 4280 | LLSYTGAR | Christoph Kreer et al., |
| | | | | | | G9 | | V | 2020 |
| | | | | | | | | | (https://doi.org/10.1016/ |
| | | | | | | | | | j.cell.2020.06.044) |
| | | | | | | MnC2t1p1_ | 4281 | QQANSFP | Christoph Kreer et al., |
| | | | | | | A3 | | GT | 2020 |
| | | | | | | | | | (https://doi.org/10.1016/ |
| | | | | | | | | | j.cell.2020.06.044) |
| | | | | | | MnC2t1p1_ | 4282 | QQANSFP | Christoph Kreer et al., |
| | | | | | | C5 | | GT | 2020 |
| | | | | | | | | | (https://doi.org/10.1016/ |
| | | | | | | | | | j.cell.2020.06.044) |
| | | | | | | MnC2t2p1_ | 4283 | QQSYSTLY | Christoph Kreer et al., |
| | | | | | | C11 | | S | 2020 |
| | | | | | | | | | (https://doi.org/10.1016/ |
| | | | | | | | | | j.cell.2020.06.044) |
| | | | | | | MnC4t1p1_ | 4284 | LQHNTYPF | Christoph Kreer et al., |
| | | | | | | A10 | | T | 2020 |
| | | | | | | | | | (https://doi.org/10.1016/ |
| | | | | | | | | | j.cell.2020.06.044) |
| | | | | | | MnC4t1p1_ | 4285 | HQYGSSP | Christoph Kreer et al., |
| | | | | | | A11 | | WT | 2020 |
| | | | | | | | | | (https://doi.org/10.1016/ |
| | | | | | | | | | j.cell.2020.06.044) |
| | | | | | | MnC4t2p1_ | 4286 | QQGNSFPF | Christoph Kreer et al., |
| | | | | | | B3 | | T | 2020 |
| | | | | | | | | | (https://doi.org/10.1016/ |
| | | | | | | | | | j.cell.2020.06.044) |
| | | | | | | MnC4t2p1_ | 4287 | LQHNTYPF | Christoph Kreer et al., |
| | | | | | | D10 | | T | 2020 |
| | | | | | | | | | (https://doi.org/10.1016/ |
| | | | | | | | | | j.cell.2020.06.044) |
| | | | | | | MnC4t2p1_ | 4288 | QQGNSFPF | Christoph Kreer et al., |
| | | | | | | E6 | | T | 2020 |
| | | | | | | | | | (https://doi.org/10.1016/ |
| | | | | | | | | | j.cell.2020.06.044) |
| | | | | | | MnC4t2p1_ | 4289 | LQHNTYPF | Christoph Kreer et al., |
| | | | | | | F5 | | T | 2020 |
| | | | | | | | | | (https://doi.org/10.1016/ |
| | | | | | | | | | j.cell.2020.06.044) |
| | | | | | | MnC4t2p2_ | 4290 | QHFGTSSV | Christoph Kreer et al., |
| | | | | | | A4 | | T | 2020 |
| | | | | | | | | | (https://doi.org/10.1016/ |
| | | | | | | | | | j.cell.2020.06.044) |
| | | | | | | MnC5t2p1_ | 4291 | QQYGSSP | Christoph Kreer et al., |
| | | | | | | G1 | | WT | 2020 |
| | | | | | | | | | (https://doi.org/10.1016/ |
| | | | | | | | | | j.cell.2020.06.044) |
| | | | | | | MR10 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR14 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR17 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR17_K56W | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR17_K99W | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR17_K99Y | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR2 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR3 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR4 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR6 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR7 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | MR8 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | Nb11 | | ND | Michael Schoof et al., |
| | | | | | | | | | 2020 |
| | | | | | | | | | (https://www.biorxiv.org/ |
| | | | | | | | | | content/10.1101/ |
| | | | | | | | | | 2020.08.08.238469v1.full. |
| | | | | | | | | | pdf) |
| | | | | | | Nb11-59 | | ND | Junwei Gai et al., 2020 |
| | | | | | | | | | (https://www.biorxiv.org/ |
| | | | | | | | | | content/10.1101/ |
| | | | | | | | | | 2020.08.09.242867v1.full. |
| | | | | | | | | | pdf) |
| | | | | | | Nb3 | | ND | Michael Schoof et al., |
| | | | | | | | | | 2020 |
| | | | | | | | | | (https://www.biorxiv.org/ |
| | | | | | | | | | content/10.1101/ |
| | | | | | | | | | 2020.08.08.238469v1.full. |
| | | | | | | | | | pdf) |
| | | | | | | Nb3-bi | | ND | Michael Schoof et al., |
| | | | | | | | | | 2020 |
| | | | | | | | | | (https://www.biorxiv.org/ |
| | | | | | | | | | content/10.1101/ |
| | | | | | | | | | 2020.08.08.238469v1.full. |
| | | | | | | | | | pdf) |
| | | | | | | Nb3-tri | | ND | Michael Schoof et al., |
| | | | | | | | | | 2020 |
| | | | | | | | | | (https://www.biorxiv.org/ |
| | | | | | | | | | content/10.1101/ |
| | | | | | | | | | 2020.08.08.238469v1.full. |
| | | | | | | | | | pdf) |
| | | | | | | Nb4-43 | | ND | Junwei Gai et al., 2020 |
| | | | | | | | | | (https://www.biorxiv.org/ |
| | | | | | | | | | content/10.1101/ |
| | | | | | | | | | 2020.08.09.242867v1.full. |
| | | | | | | | | | pdf) |
| | | | | | | Nb6 | | ND | Michael Schoof et al., |
| | | | | | | | | | 2020 |
| | | | | | | | | | (https://www.biorxiv.org/ |
| | | | | | | | | | content/10.1101/ |
| | | | | | | | | | 2020.08.08.238469v1.full. |
| | | | | | | | | | pdf) |
| | | | | | | Nb6-tri | | ND | Michael Schoof et al., |
| | | | | | | | | | 2020 |
| | | | | | | | | | (https://www.biorxiv.org/ |
| | | | | | | | | | content/10.1101/ |
| | | | | | | | | | 2020.08.08.238469v1.full. |
| | | | | | | | | | pdf) |
| | | | | | | NIH- | | N/A | Thomas Esparza et al., |
| | | | | | | CoVnb- | | | 2020 |
| | | | | | | 101 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 102 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 103 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 104 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 105 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 106 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 107 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 108 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 109 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 110 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 111 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 112 | | | (https://www.biorxiv.org/ |
| | | | | | | | | | content/10.1101/ |
| | | | | | | | | | 2020.07.24.219857v1) |
| | | | | | | NIH- | | N/A | Thomas Esparza et al., |
| | | | | | | CoVnb- | | | 2020 |
| | | | | | | 113 | | | (https://www.biorxiv.org/ |
| | | | | | | | | | content/10.1101/ |
| | | | | | | | | | 2020.07.24.219857v1) |
| | | | | | | P2B-2F6 | 4292 | SSYAGSNN | Bin Ju et al., 2020 |
| | | | | | | | | LV | (https://www.nature.com/ |
| | | | | | | | | | articles/s41586-020- |
| | | | | | | | | | 2380-z) |
| | | | | | | S110 | 4293 | MQGTHW | Dora Pinto et al., 2020 |
| | | | | | | | | PPT | (https://www.nature.com/ |
| | | | | | | | | | articles/s41586-020- |
| | | | | | | | | | 2349-y) |
| | | | | | | S124 | 4294 | QQSYSTPP | Dora Pinto et al., 2020 |
| | | | | | | | | T | (https://www.nature.com/ |
| | | | | | | | | | articles/s41586-020- |
| | | | | | | | | | 2349-y) |
| | | | | | | S303 | 4295 | QQYDTYS | Dora Pinto et al., 2020 |
| | | | | | | | | WT | (https://www.nature.com/ |
| | | | | | | | | | articles/s41586-020- |
| | | | | | | | | | 2349-y) |
| | | | | | | S304 | 4296 | QQSYVSPT | Dora Pinto et al., 2020 |
| | | | | | | | | | (https://www.nature.com/ |
| | | | | | | | | | articles/s41586-020- |
| | | | | | | | | | 2349-y) |
| | | | | | | S306 | 4297 | QQRSNWP | Dora Pinto et al., 2020 |
| | | | | | | | | PGCS | (https://www.nature.com/ |
| | | | | | | | | | articles/s41586-020- |
| | | | | | | | | | 2349-y) |
| | | | | | | S309 | 4298 | QQHDTSLT | Dora Pinto et al., 2020 |
| | | | | | | | | | (https://www.nature.com/ |
| | | | | | | | | | articles/s41586-020- |
| | | | | | | | | | 2349-y) |
| | | | | | | S310 | 4299 | CSYAGSDT | Dora Pinto et al., 2020 |
| | | | | | | | | VI | (https://www.nature.com/ |
| | | | | | | | | | articles/s41586-020- |
| | | | | | | | | | 2349-y) |
| | | | | | | S315 | 4300 | QSADSSGT | Dora Pinto et al., 2020 |
| | | | | | | | | V | (https://www.nature.co |
| | | | | | | | | | m/articles/s41586-020- |
| | | | | | | | | | 2349-y) |
| | | | | | | Sb#1 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#10 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#11 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#12 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#13 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#14 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#15 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#16 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#17 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#18 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#19 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#2 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#20 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#21 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#22 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#23 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#25 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#26 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#27 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#28 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#29 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#3 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#30 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#31 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#32 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#33 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#34 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#35 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#36 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#37 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#38 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#39 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#4 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#40 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#41 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#42 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#45 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#46 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#47 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#48 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#49 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#5 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#50 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#51 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#52 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#53 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#55 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#56 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#58 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#59 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#6 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#61 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#62 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#63 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#64 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#65 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#66 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#67 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#68 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#69 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#7 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#71 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#8 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb#9 | | N/A | Justin Walter et al., |
| | | | | | | | | | 2020 |
| | | | | | | | | | (https://www.biorxiv.org/ |
| | | | | | | | | | content/10.1101/ |
| | | | | | | | | | 2020.04.16.045419v2) |
| | | | | | | Sb100 | | N/A | Tania Custodia et al., |
| | | | | | | | | | 2020 |
| | | | | | | | | | (https://www.biorxiv.or |
| | | | | | | | | | g/content/10.1101/202 |
| | | | | | | | | | 0.06.23.165415v1.full.p |
| | | | | | | | | | df+html) |
| | | | | | | Sb12 | | N/A | Tania Custodia et al., |
| | | | | | | | | | 2020 |
| | | | | | | | | | (https://www.biorxiv.or |
| | | | | | | | | | g/content/10.1101/202 |
| | | | | | | | | | 0.06.23.165415v1.full.p |
| | | | | | | | | | df+html) |
| | | | | | | Sb13 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb15 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb16 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb17 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb2 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb21 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb22 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb23 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb25 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb27 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb28 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb30 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb32 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb37 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb38 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb39 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb40 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb42 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb43 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb45 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb46 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb47 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb5 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb50 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb52 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb54 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb56 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb57 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb58 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb6 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb60 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb61 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb62 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb63 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb67 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb7 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb71 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb75 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb76 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb78 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb8 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb83 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb84 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb85 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb88 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb9 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb90 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb93 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb94 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb95 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | Sb97 | | N/A | Tania Custodia et al., |
| | | | | | | | | | 2020 |
| | | | | | | | | | (https://www.biorxiv.org/ |
| | | | | | | | | | content/10.1101/ |
| | | | | | | | | | 2020.06.23.165415v1.full. |
| | | | | | | | | | pdf + html) |
| | | | | | | SR1 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | SR13 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | SR18 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | SR31 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | SR34 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | SR38 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | SR4 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | SR5 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | SR7 | | N/A | Tingting Li et al., 2020 |
| | | | | | | | | | (https://www.biorxiv.org/ |
| | | | | | | | | | content/10.1101/ |
| | | | | | | | | | 2020.06.09.143438v1) |
| | | | | | | Ty1 | | N/A | Leo Hanke et al., 2020 |
| | | | | | | | | | (https://www.biorxiv.org/ |
| | | | | | | | | | content/10.1101/ |
| | | | | | | | | | 2020.06.02.130161V1) |
| | | | | | | VH-Fc- | | ND | Colton J. Bracken et al., |
| | | | | | | B01 | | | 2020 |
| | | | | | | | | | (https://www.biorxiv.org/ |
| | | | | | | | | | content/10.1101/ |
| | | | | | | | | | 2020.08.08.242511v1.full. |
| | | | | | | | | | pdf) |
| | | | | | | VH2-A01- | | ND | Colton J. Bracken et al., |
| | | | | | | B01 | | | 2020 |
| | | | | | | | | | (https://www.biorxiv.org/ |
| | | | | | | | | | content/10.1101/ |
| | | | | | | | | | 2020.08.08.242511v1.full. |
| | | | | | | | | | pdf) |
| | | | | | | VH2-A01- | | ND | Colton J. Bracken et al., |
| | | | | | | B02 | | | 2020 |
| | | | | | | | | | (https://www.biorxiv.org/ |
| | | | | | | | | | content/10.1101/ |
| | | | | | | | | | 2020.08.08.242511v1.full. |
| | | | | | | | | | pdf) |
| | | | | | | VH3-B01 | | ND | Colton J. Bracken et al., |
| | | | | | | | | | 2020 |
| | | | | | | | | | (https://www.biorxiv.org/ |
| | | | | | | | | | content/10.1101/ |
| | | | | | | | | | 2020.08.08.242511v1.full. |
| | | | | | | | | | pdf) |
| | | | | | | VHH-72 | | N/A | Daniel Wrapp et al., |
| | | | | | | | | | 2020 |
| | | | | | | | | | (https://www.sciencedirect. |
| | | | | | | | | | com/science/article/ |
| | | | | | | | | | pii/S0092867420304943) |
| | | | | | | W23UACh | | N/A | Guillermo Nieto et al., |
| | | | | | | | | | 2020 |
| | | | | | | | | | (https://www.biorxiv.org/ |
| | | | | | | | | | content/10.1101/ |
| | | | | | | | | | 2020.06.09.137935v1) |
| | | | | | | W25UACh | | N/A | Guillermo Nieto et al., |
| | | | | | | | | | 2020 |
| | | | | | | | | | (https://www.biorxiv.org/ |
| | | | | | | | | | content/10.1101/ |
| | | | | | | | | | 2020.06.09.137935v1) |

### EXAMPLES

The examples in this section are offered by way of illustration, and not by way of limitation.

### General Method: Plasmid Construction:

To construct single-gene expression plasmids, ORF (with Kosak and stop codon) of interested gene was used to simply replace existing gene at BstEII-BglII of BV2 vector (Kan resistant plasmid). For constructing multi-gene expression plasmids, the second ORF was inserted into expression-shuttle vector (Amp resistant plasmid) at BstEII-BglII. The expression cassette of resulting plasmid was then digested (RI-XbaI) and inserted into existing single expression plasmid at RI-NheI. This process yielded two expression cassette vectors with RI-NheI site available to another round of insertion.

### Example 1: Subcutaneous Injection of Plasmids in Mice with Successful Serum Expression

This Example describes subcutaneous injections of plasmids in mice with successful serum expression.

### Methods:

Three mice per group were injected subcutaneously (SC) with plasmid DNA encoding either an anti-CoV-2 mAb (experiment 375) or hGCSF (experiment 382). Mice were given subcutaneous injections in the interscapular region (scap) or shaved and given subcutaneous injections targeted to the 4^{th} and 5^{th} inguinal mammary fat pads (ing). DNA was suspended either in normal Lactated Ringer's or with hypertonic Ringer's (3%) (both without a particular carrier agent). Some mice were also given 1000 nmol DMPC neutral liposomes with the DNA, or low dose Chloroquine.

### Results:

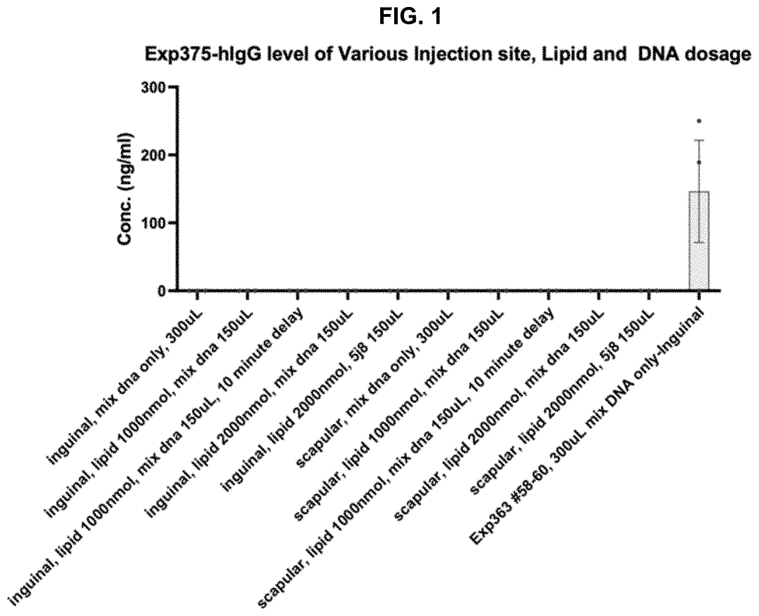

Results of experiment 375 (anti-CoV-2 mAb) are shown in FIG. 1, showing serum levels of protein expression. A significant number of different mouse groups each received a different subcutaneous regimen as described in the methods as well as in FIG. 1. Mice 58 and 59 from experiment 375 each produced significant serum levels of an anti-SARS-CoV-2 neutralizing mAb 24 hours after subcutaneous injection. In contrast, none of the other subcutaneous injected mice produce detectable levels of this same mAb. Mice 58 and 59 produced detectable levels plasmid DNA encoded protein after subcutaneous injection. Furthermore, each of these two mice produced sufficiently high serum protein levels of the DNA therapeutic protein to produce a therapeutic anti-covid effect. It is noted that all the mice without significant expression had weights of between about 30 and 40 grams (considered non-overweight), while mice 58 and 59 had weights of between about 42 and 53 grams (considered grossly obese). It is believed that showing significant serum protein levels after plasmid injection subcutaneously in mice, without an expression aid of any type, is a fundamental, World changing achievement that may progressively replace all bio reactor produced recombinant proteins including monoclonal antibodies worldwide.

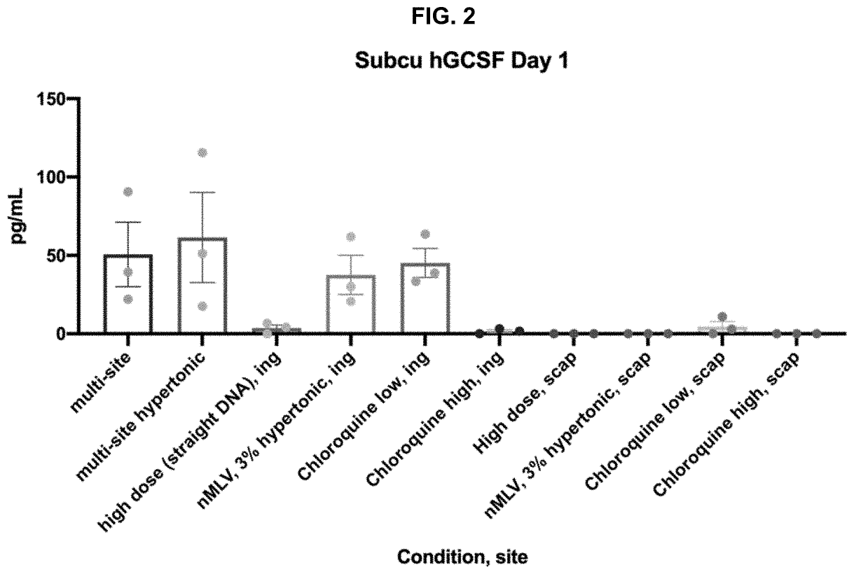

Results of experiment 382 (hGCSF) are shown in FIG. 2, showing serum levels of protein expression. A significant number of different mouse groups each received a different subcutaneous regimen as described in the methods as well as in FIG. 2. A variety of different mouse groups from experiment 382 each produced significant serum levels of the human G-CSF protein 24 hours after subcutaneous injection. These included mouse groups that received hG-CSF plasmid DNA alone (no added expression aids), hG-CSF plasmid DNA in hypertonic saline (3%) (no added expression aids), hG-CSF plasmid DNA mixed with low or high dose chloroquine hG-CSF plasmid, and/or DNA mixed with neutral MLV liposomes. Experiment 382 demonstrates that a variety of different subcutaneous injection approaches, including plasmid DNA alone or plasma DNA mixed with either chloroquine, hypertonic saline 5 or neutral MLV liposomes can each produce significant serum levels of the human G-CSF protein 24 hours after subcutaneous injection. Experiment 382 shows that a variety of different compositions containing either plasmid DNA alone or plasmid DNA with a variety of different drugs or diluents as well as with neutral MLV liposomes alone each produces significant serum levels of the plasmid DNA encoded protein product.

### Example 2: Subcutaneous Injection of Plasmids in Rats with Successful Serum Expression

This Example describes subcutaneous injections of plasmids in rats with successful serum expression.

### Methods:

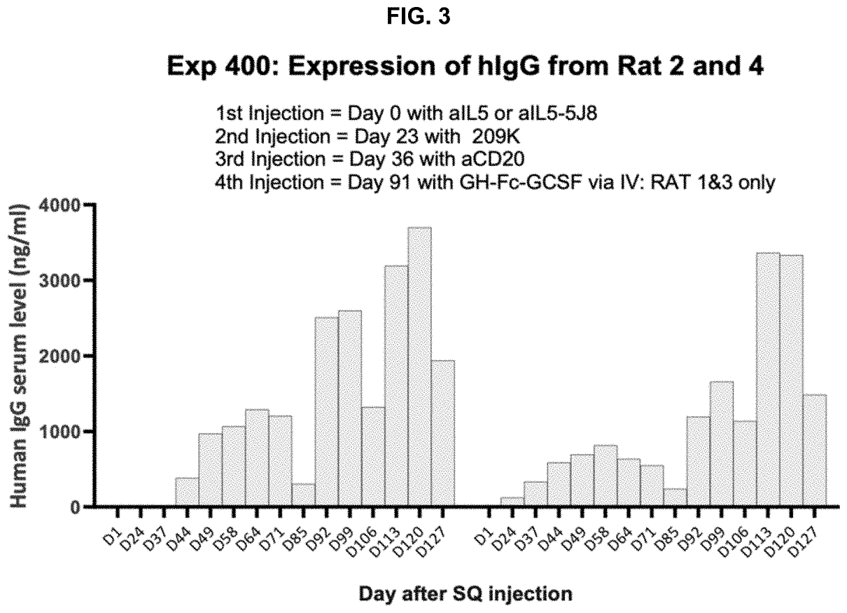

On day 0, rats were given subcutaneous injection of plasmid DNA encoding anti-human IL-5 mAb (17 mg, left) or anti-IL5 mAb plus anti-a5J8 mAb (5 mg, right). On day 23, rats were both injected subcutaneously with 25 mg plasmid encoding anti-CoV-2 mAb 209K, one with hyaluronidase (right side of FIG. 3) and one without any expression aid (left side of FIG. 3). On day 36, both rats were injected subcutaneously with 20 mg plasmid DNA encoding anti-CD20 (rituximab).

### Results:

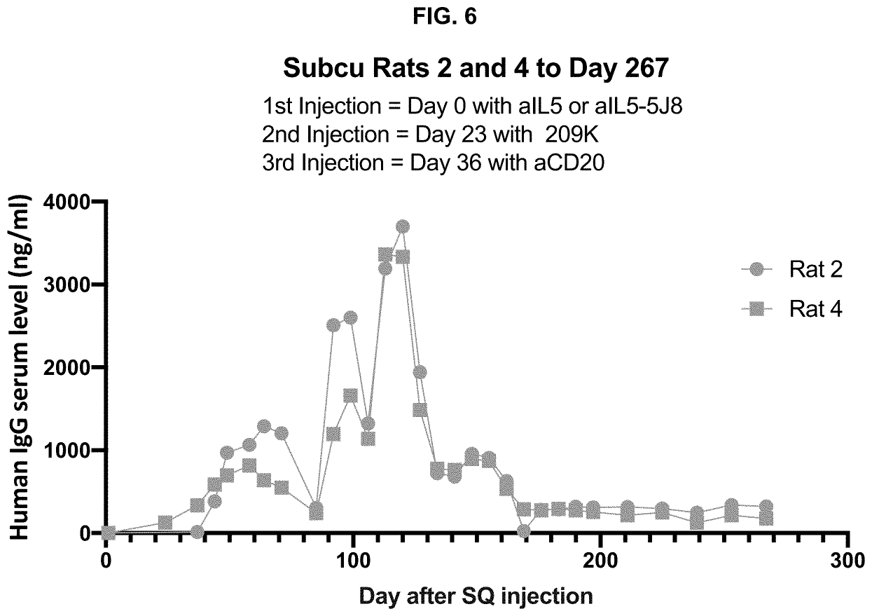

The results are shown in FIG. 3 and FIG. 6. Two different rat groups each received the same subcutaneous regimen as described in the methods as well as in FIG. 3 and FIG. 6. From day 44 on (day 267 was the most recent weekly blood draw), serum anti-human IgG levels of anti-CoV2 antibody were above one microgram per ml. These serum levels directly correlated with essentially 90 to 100 percent neutralization of the CoV-2 virus and for at least 127 days after a single subcutaneous injection. 127 days is the human equivalent of approximately 25 years. FIG. 6 further shows that the antibody level in serum peaked at about 115 days after administration and remained detectable for at least 267 days after initial administration.

This example shows two rats (#2 and #4) who produced detectable serum levels of plasmid DNA encoded protein following subcutaneous injection. Each of these two rats produced sufficiently high serum mAb protein levels of the plasmid DNA-encoded anti-CoV-2 mAb therapeutic protein to produce nearly 100% anti-CoV-2 neutralization. It is believed that showing significant serum protein levels after plasmid injection subcutaneously in rats, with and without an expression aid of any type, is an important achievement.

### Example 3: Subcutaneous Injection of Plasmids in Rats with Successful Serum Expression

This Example describes subcutaneous injections of plasmids in rats with successful serum expression with or without various pre-treatments.

### Methods:

Two rats (rats #1 and #2) were given IV pre-injections of liposomes composed of 4400 nmol DOTAP SUV with 2.5% dex palmitate and 2000 nmol DMPC MLV with 5% dex palmitate, followed by IV injection of 400 ug plasmid DNA encoding rituximab. This was followed by subcutaneous injection of 20 mg plasmid DNA encoding 5J8 in 3% hypertonic saline with hyaluronidase. The third rat (#3) was given 5 IP injection of an anti-CD20 antibody, followed by the same subcutaneous injection as the first two. Rat #4 was given IP injection of rituximab followed by the same subcutaneous injection as previous. Note that there is no cross-reactivity between the serum anti-human CD20 mAb ELISA and the serum anti-human 5J8 mAb used to measure serum levels of these two different mAbs

In addition, rats #5-8 were given subcutaneous injections only of the same liposomes as in rats 1 and 2, with the total divided into 7 injection sites. This was followed by subcutaneous injection of 20 mg plasmid DNA encoding 5J8. All subcutaneous injections were divided into 7 sites: the ₃rd 4th _{and 5}th mammary fat pads, and the interscapular region.

### Results:

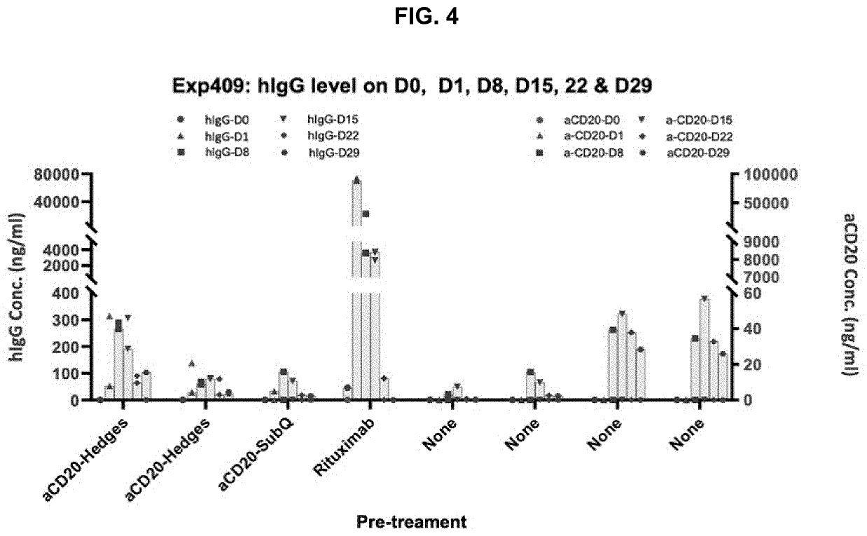

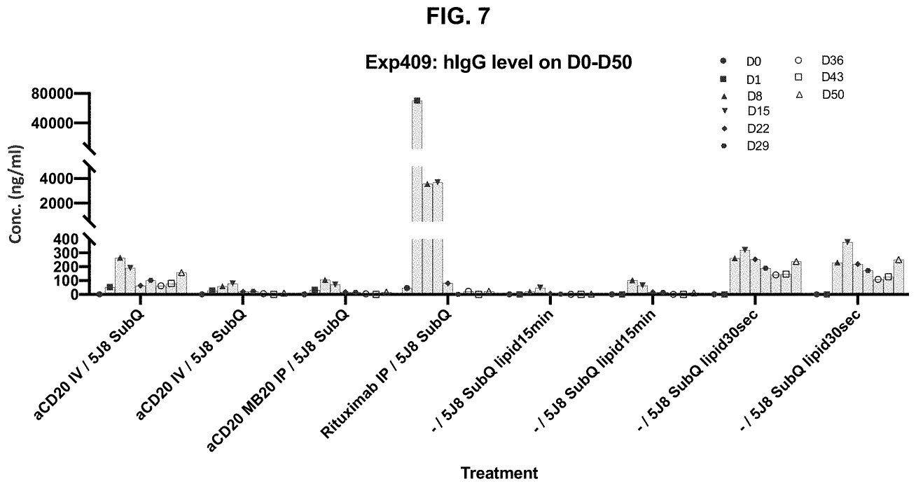

The results are shown in FIG. 4 and FIG. 7. A total of eight different rat groups each received one of the eight different subcutaneous regimens as described in the methods and above. All the rat groups receiving plasmid DNA encoding either of the two different mAb proteins showed detectable mAb serum protein levels. These results further confirm the ability of rat administered subcutaneous plasmid DNA to produce significant ongoing DNA plasmid encoded serum proteins in injected rats. FIG. 7 further shows long-term expression of protein in serum, which remained detectable for at least 50 days.

### Example 4: Subcutaneous Injection of Plasmids in Rats with Successful Serum Expression, With and Without, Lipid Pre-Treatment

This Example describes subcutaneous injections of plasmids in rats with successful serum expression with or without Lipid pre-treatment.

### Methods:

Two rats per group were injected subcutaneously with 5, 15, 25, or 40 mg plasmid DNA encoding an anti-CoV2 antibody (without expression aid). Two rats were also injected with 25 mg the same DNA but after being given liposome injections of 4,400 nmol of pure DOTAP SUV mixed with 2.5% dex palmitate and 2000 nmol DMPC neutral MLV with 5% dex palmitate.

### Results:

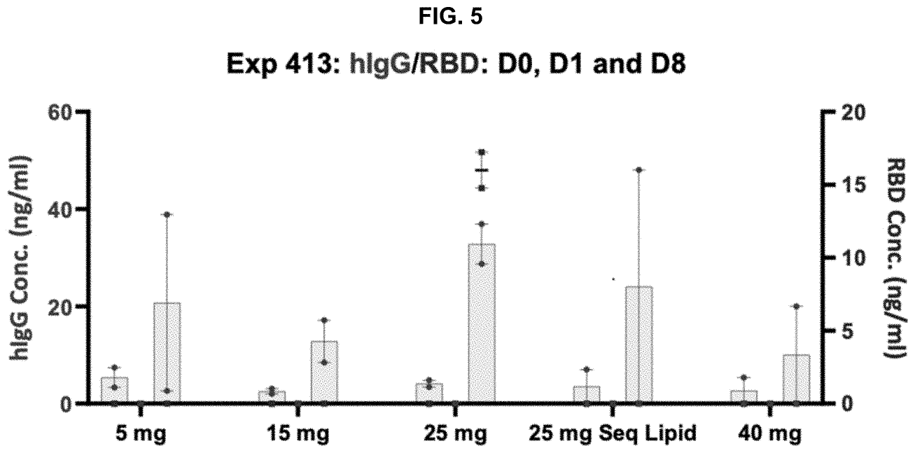

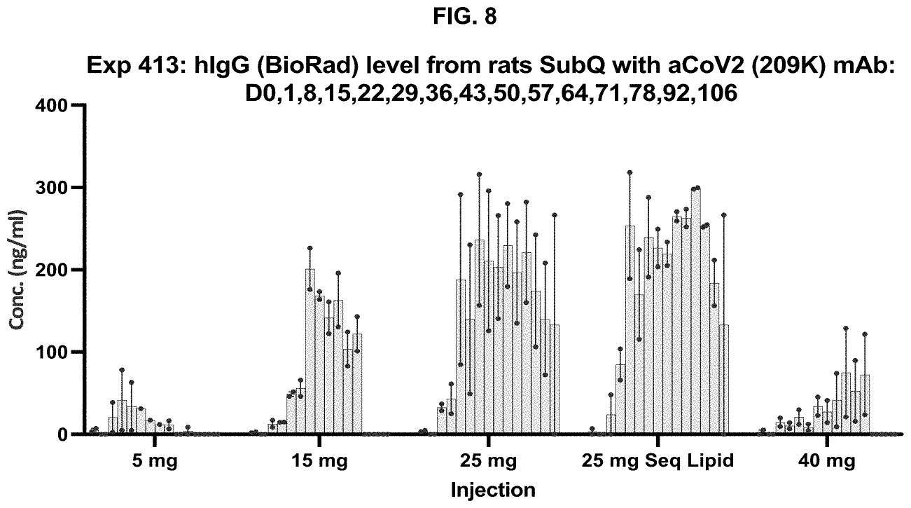

The results are shown in FIG. 5 and FIG. 8. A total of five different rat groups of two each received one of five different subcutaneous DNA does regimens as described in the methods. All five rat groups receiving plasmid DNA encoding an anti-CoV-2 mAb protein showed detectable anti-CoV-2 mAb serum protein levels on days one and eight after subcutaneous injection. This includes not only the four groups receiving subcutaneous plasmid DNA alone but also the fifth group that received a sequential subcutaneous injection, of cationic SUV liposomes mixed with neutral MLV liposomes followed 30 seconds later by subcutaneous injection of plasmid DNA only. These experimental results demonstrate that sequential liposome then plasmid DNA injection produced significant serum levels of the plasmid DNA encoded protein at very early time points after injection. FIG. 8 further shows that the long-term expression of proteins in serum, which was detectable for at least 106 days.

### Example 5: In Vitro Transfection of Human Preadipocyte Primary Cells

This example describes in vitro transfection of human derived preadipocyte cells in culture with successful expression.

### Methods:

Preadiopcytes were grown and then plated for transfection at a concentration of 0.25×10⁵ cells per well in a 24-well plate. The cells were transfected with 1 or 5μg plasmid DNA encoding an anti-CoV-2 mAb complexed with either 2.7 μl Expifectamine (ThermoFisher)/μg DNA, 1.334 or 3.335 molar ratio of PEI/μg DNA, or 10, 20, or 30 nmol DOTAP/μg DNA. Thin-film transfections were performed by adding the DNA-lipid complexes to already plated cells, while direct transfections were performed by combining the DNA-lipid complexes with the cell suspension before adding to the cell culture plate. The media was collected every three days and assayed for hIgG levels.

### Results:

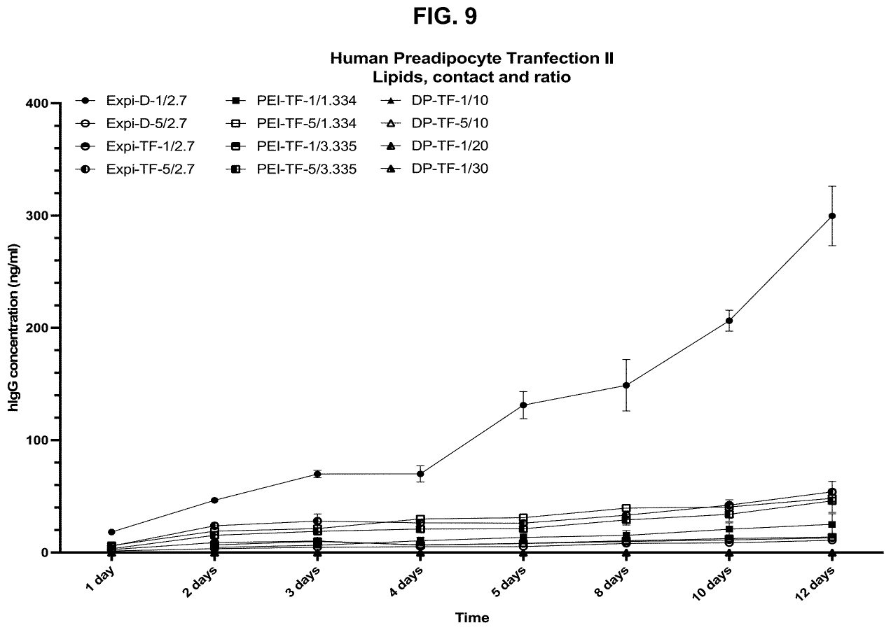

The results are shown in FIG. 9, showing protein expression levels. The best condition identified was the thin-film transfection with 1 μg DNA-2.7 μl Expifectamine which consistently showed the highest expression levels from day 1 to day 12. In contrast, the cells transfected with the different DNA-DOTAP ratios did not express from day 1 to day 12. The other expifectamine and PEI groups showed consistent low-level expression across the timeline. These results show that the preadipocyte cells were highly transfectable.

### Example 6: In Vitro Transfection of Human Preadipocyte Primary Cells in Various Stages of Differentiation into Mature Adipocytes

This example describes the in vitro transfection of the various stages of differentiation of human derived preadipocytes into fully mature adipocytes.

### Methods:

Human derived preadipocytes were differentiated into mature adipocytes over the course of 21 days. The cells were transfected every three days, starting with preadipocytes and ending with the mature adipocytes, with a plasmid DNA vector encoding an anti-CoV-2 mAb complexed with Expifectamine (ThermoFisher) at a ratio of 5 μg DNA:2.7 μl lipid. The media was changed every three days and assayed for hIgG levels.

### Results:

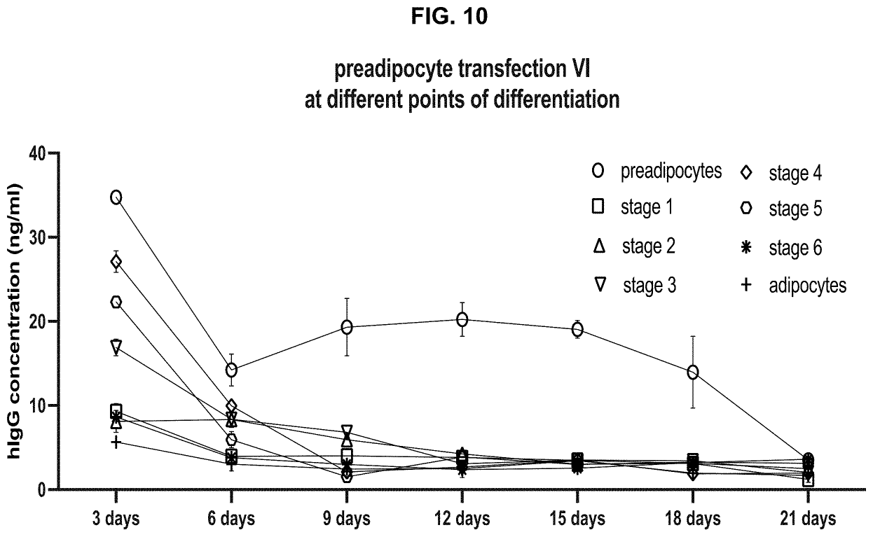

The results are shown in FIG. 10, showing protein expression levels following transfection of the various stages of preadipocyte differentiation into mature adipocytes. The most efficiently transfected stage was the preadipocytes as seen by the elevated expression levels of hIgG compared to the other stages of differentiating cells. The final mature adipocytes were the least transfectable as shown by the low levels of hIgG expression detected at day 3 (5 ng/ml) all the way up to day 21. There was reduced expression levels for stages 3, 4, and 5 compared to the preadipocyte group, but compared to the mature adipocytes the expression level was higher on days 3 and 6. These results show that targeting a mixed population of preadipocytes along with maturing adipocytes resulted in high levels of protein expression. Additionally, transfected preadipocytes maintained their expression levels as they mature into adipocytes.

### Example 7: In Vitro Transfection of Mature Human Adipocytes.

This example describes the in vitro transfection of matures human adipocytes.

### Methods:

Human derived preadipocytes were differentiated into mature adipocytes for 21 days before they were transfected with a plasmid DNA vector encoding an anti-CoV-2 mAb complexed with either 0.54 μl Expifectamine (ThermoFisher)/μg DNA, 1.334 molar ratio of PEI/μg DNA, or 2 nmol DOTAP/μg DNA. Transfections were done by either thin-film or direct transfection. Thin-film transfections were performed by adding the DNA-lipid complexes to already plated adipocytes, while direct transfections were performed by combining the DNA-lipid complexes and pipetting the mixture up and down in the well with the attached adipocytes to generate a single cell suspension. The media was collected every two days and assayed for hIgG levels.

### Results:

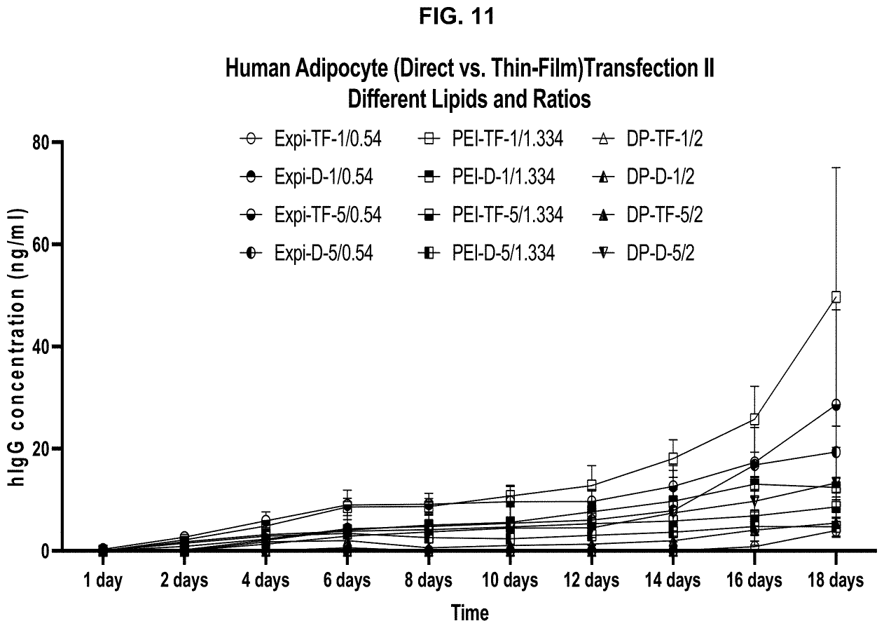

The results are shown in FIG. 11. The highest expression levels were seen in the thin-film PEI group transfected with 1 μg of plasmid DNA and a 1.334 molar ratio of PEI. Overall expression in the adipocytes took at least one week to start increasing in every group tested. The DOTAP groups were the least successful for each method and DNA-lipid ratio tested. The Expifectamine treated groups with the highest expression at day 18 were those transfected with the direct transfection method (both 1 and 5 μg plasmid DNA), while the other expifectamine thin-film groups did not express as highly. These results show that mature human adipocytes expressed the anti-CoV-2 mAb following transfection. The expression was both delayed and reduced compared to the predipocytes in previous examples.

### Example 8: Use of Obese Mice for Subcutaneous Injection

### Methods:

Three obsess mice (ob-/ob- or B6.Cg-Lepob/J strain) per group were injected subcutaneously with 800, 1600, or 3200 μg of a plasmid encoding hGH and hGCSF in Lactated Ringer's solution. Obese mice that received injections of lactated Ringer's solution (blank) were included as the negative control.

Blood samples were taken on both 1 day and 15 days post injection from the mice injected with the plasmid and control group, and the expression level of hGH and hGCSF were assessed by ELISA. Additionally, whole blood samples collected from the Day 15 bleeds were submitted for a complete blood count to assess absolute neutrophil count (ANC).

### Results:

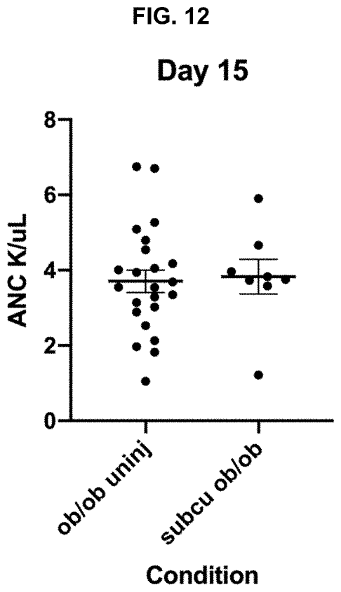

The obese mice, while having larger fat deposits in both visceral and subcutaneous fat compartments, did not express hGH and hGCSF faster or at higher levels as compared to the control group. The hGH and hGCSF ELISAs returned values that were indistinguishable from background (data not shown). The ANC count using Day 15 bleeds also did not differ between un-injected mice and subcutaneously injected mice (FIG. 12).

### Example 9: Injection of hGLA in 1 mL Volume

### Methods:

A total of 18 mice were group into 6 groups of three mice per group. The groups of three mice were each injected subcutaneously with 300, 900, 1800, or 3200 μg of a plasmid encoding hGLA-1×L-hyFc in 1 mL of Lactated Ringer's solution or 1 mL of hypotonic (.5%) Ringer's solution. The total 1 mL Volume was divided into 200 uL per inguinal fat pad and 600 uL into the subscapular region for each injected mice. Mice were bled at day 1 and then every 7 or 14 days thereafter and hGLA expression level assessed by ELISA.

### Results:

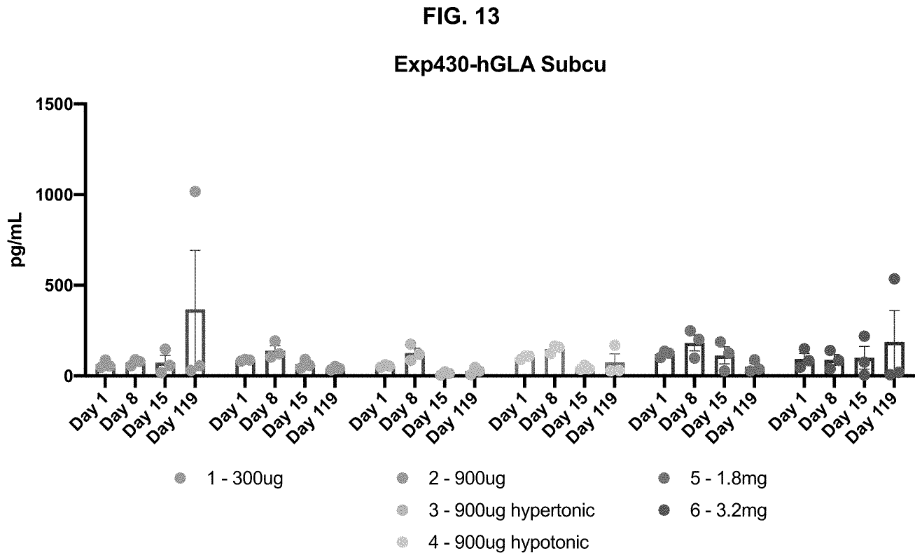

The results are shown in FIG. 13. Using the 1 mL volume, 2 of the 18 mice still expressed significant levels of hGLA at day 119.

### Example 10: Injection of hGLA in Smaller Volumes

### Methods:

Mice were treated with antibodies against CD4 and B220 to deplete B and T cell populations. A first group of five mice were injected subcutaneously with 800 μg of a plasmid encoding hGLA-1×L-hyFc in 800 μL Lactated Ringer's solution. A second group of five mice were injected subcutaneously with 800 μg of a plasmid encoding hGLA-1×L-hyFc in 200 μL Lactated Ringer's solution. A third group of five mice were injected subcutaneously with 1600 μg of a plasmid encoding hGLA-1×L-hyFc in 800 μL Lactated Ringer's solution. A fourth group of five mice were injected subcutaneously with 1600 μg of a plasmid encoding hGLA-1×L-hyFc in 400 μL Lactated Ringer's solution. A fifth group of five mice were injected subcutaneously with 3200 μg of a plasmid encoding hGLA-1×L-hyFc in 800 μL Lactated Ringer's solution. A final sixth group of five mice were injected with 1600 μg plasmid in 800 μL hypotonic (0.5%) Ringer's solution. The groups of mice were bled at day 1 and then every 7 or 14 days thereafter and hGLA expression level assessed by ELISA.

### Results:

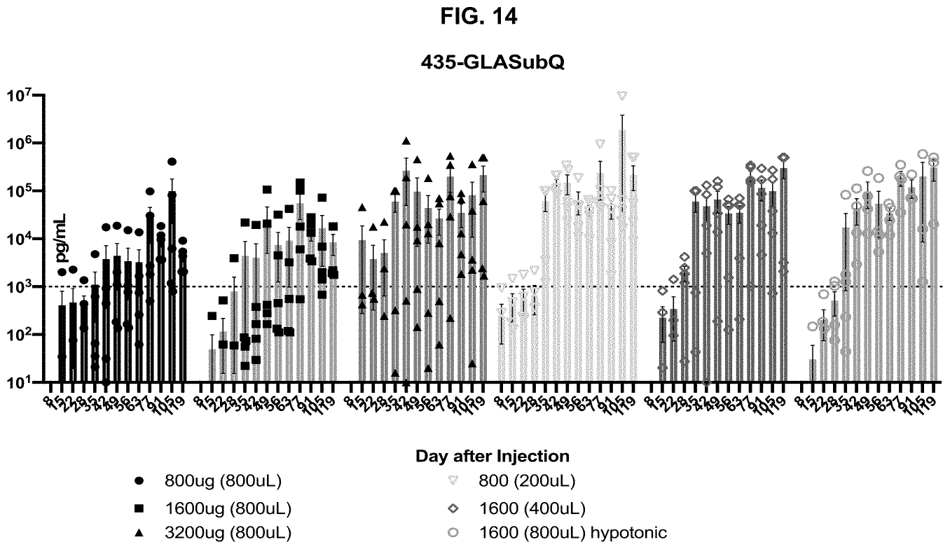

The results are shown in FIG. 14. The results indicate that 800 μg of hGLA plasmid injected one time was sufficient to produce protein expression well above the minimal therapeutic level (dotted line), starting between 22 and 28 days after injection. Smaller volumes of the same dose (group 4 vs group 1 and group 5 vs group 2) yielded higher expression. This high level of expression was maintained at least 119 days after injection.

### Example 11: Expression of Factor VIII or IX Related to Hemophilia

### Methods:

Five mice per group were treated with antibodies against CD4 and B220 to deplete B and T cell populations. Mice were injected subcutaneously with 400, 800, or 1600 μg of a plasmid encoding Factor IX in 800 μL (standard) or 1 mL (higher volume) volumes of Lactated Ringer's solution. Mice were bled at day 1 and then every 7 days thereafter and Factor IX expression level assessed by ELISA.

### Results:

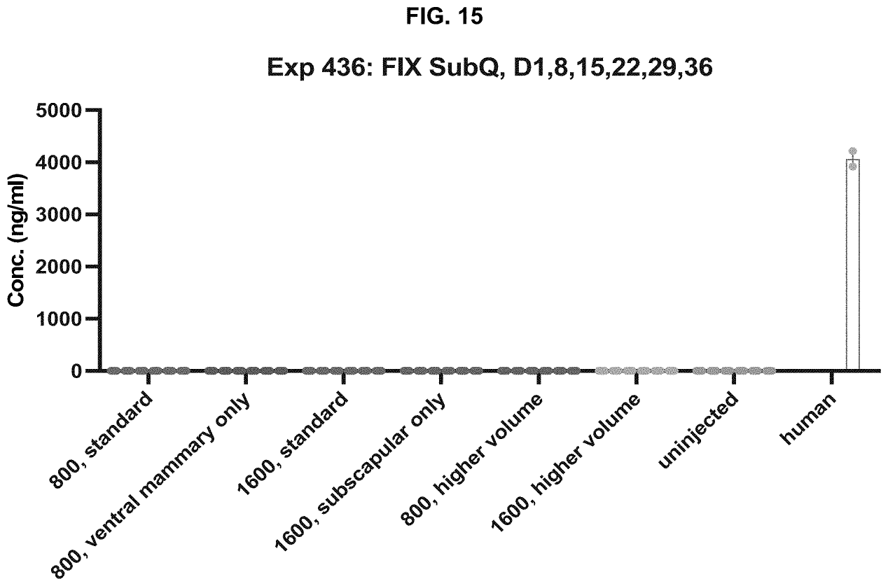

The results are shown in FIG. 15. The results indicate that Factor IX did not express when injected using the standard subcutaneous plasmid delivery that generated hGLA expression.

### Example 12: Expression of Antibody Against SARS-Cov-2 by Inguinal Injection

### Methods:

Three mice per group were injected subcutaneously with 400, 800,1600, or 3200 μg of a plasmid encoding an anti-CoV2 antibody in 100, 200, 400, or 800 μL volumes of Lactated Ringer's solution. In the final group, the volume was divided into 6 smaller injections distributed along the length of the inguinal fat pad. Mice were bled at day 1 and then every 7 or 14 days thereafter and protein expression level assessed by ELISA.

### Results:

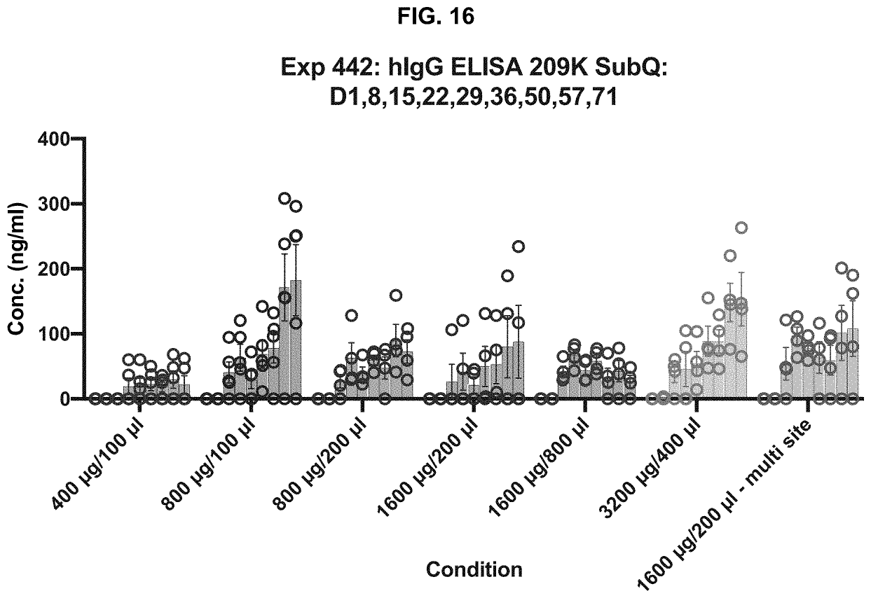

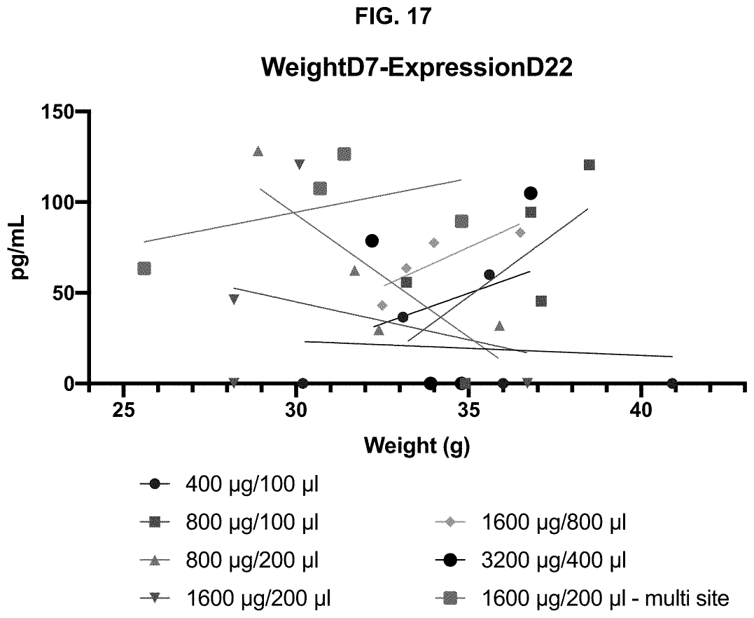

The results are shown in FIG. 16. The results indicate that anti-CoV2 antibody expressed at a low level in mice injected subcutaneously with at least 800 μg plasmid. Smaller volumes of the same dose of plasmid expressed at a slightly higher level.

Furthermore, to investigate whether the body weight of an individual mouse correlates with the expression level of the anti-CoV2 antibody in the mouse, measured expression levels of the SARS-CoV-2 antibody were plotted against body weights of test animal in FIG. 33. As shown in the figure, no clear correlation between expression level and mice weight was observed. These data demonstrate that long-term expression of the encoded protein using the present method does not require the subject to have obesity or have certain amount of body fat.

### Example 13: Mammary Fat Pad Implantation of Plasmid Encoding Anti-CoV2 Antibody

### Methods:

Three mice per group were administered with 800 μg of a plasmid encoding an anti-CoV2 antibody in 100, 200, or 400 μL volumes of Lactated Ringer's solution. Administration were made after mice were shaved and an ˜1 cm incision was made over the inguinal region to expose the fat pad and the fat pad was teased out with forceps. After injection, mice were closed with wound clips. Mice were bled at day 1 and then every 7 or 14 days thereafter and protein expression level assessed by ELISA.

### Results:

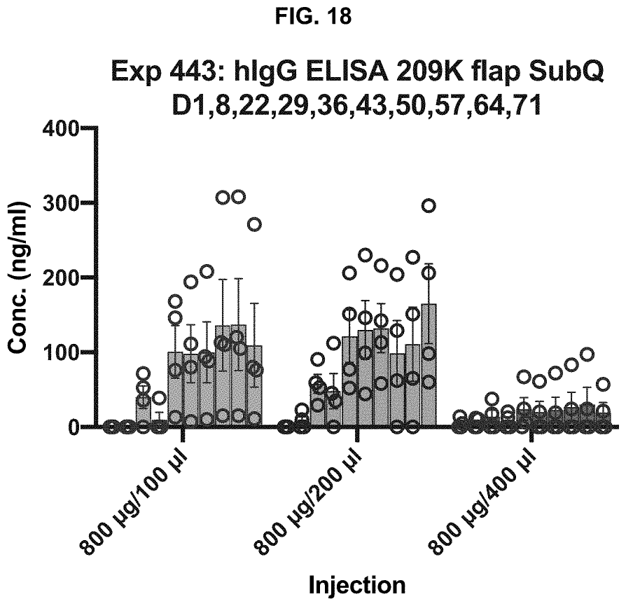

The results are shown in FIG. 18. The results indicate that anti-CoV2 antibodies expressed at a low level in mice given 800 μg plasmid via incision surgery. Mice given 100 and 200 μL volume expressed similarly to the mice given the same volumes via regular subcutaneous injection. Mice given 400 μL via incision surgery did not express significant levels of protein. Overall, less injection volume resulted in higher protein expression.

### Example 14: Expression of Anti-CoV2 Antibodies in Mice Previously Injected with Factor IX Plasmid

### Methods:

Mice that were previously injected with Factor IX were used for reinjection. Three mice per group were injected subcutaneously with 400, 800, or 1600 μg of a plasmid encoding the anti-CoV2 antibody in indicated volumes of Lactated Ringer's solution. Mice were bled at day 1 and then every 7 or 14 days thereafter and protein expression level assessed by ELISA.

### Results:

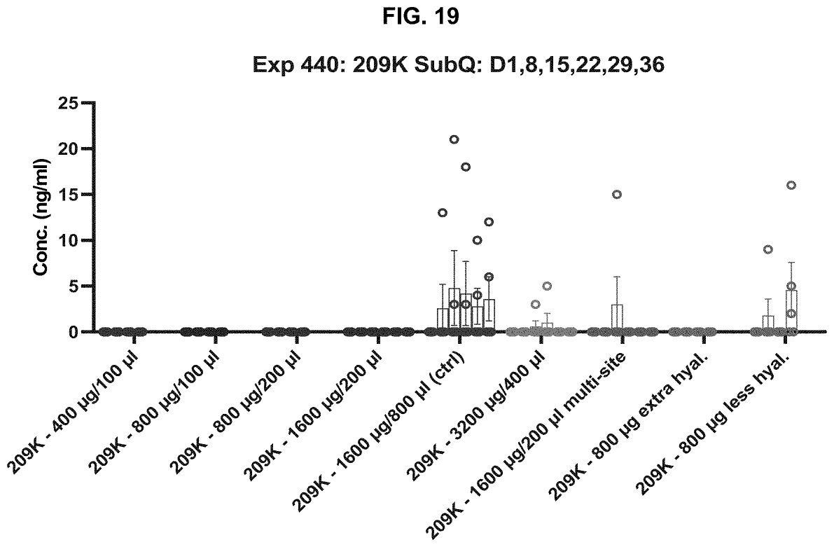

The results are shown in FIG. 19. The results indicate that anti-CoV2 antibodies did not express to appreciable levels in mice previously injected subcutaneously with a different plasmid that did not yield high expression.

### Example 15: Expression of hGLA at Various Plasmid Concentrations and Hyaluronidase Conditions

### Methods:

Three mice per group were treated with antibodies against CD4 and B220 to deplete B and T cell populations. Mice were injected subcutaneously with 800 μg of a plasmid encoding hGLA-1×L-hyFc in indicated volumes of Lactated Ringer's solution. The final group was injected with 60 Units of hyaluronidase per animal. Mice were bled at day 1 and then every 7 days thereafter and hGLA expression level assessed by ELISA.

### Results:

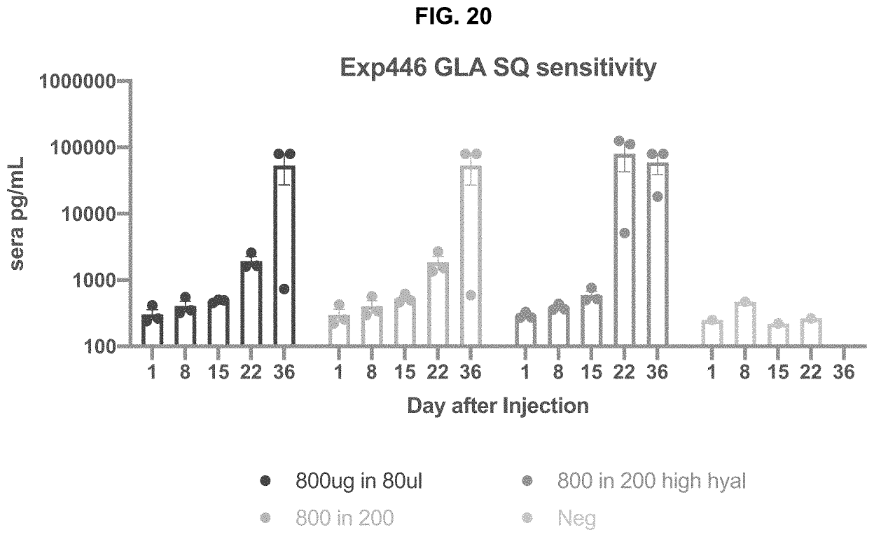

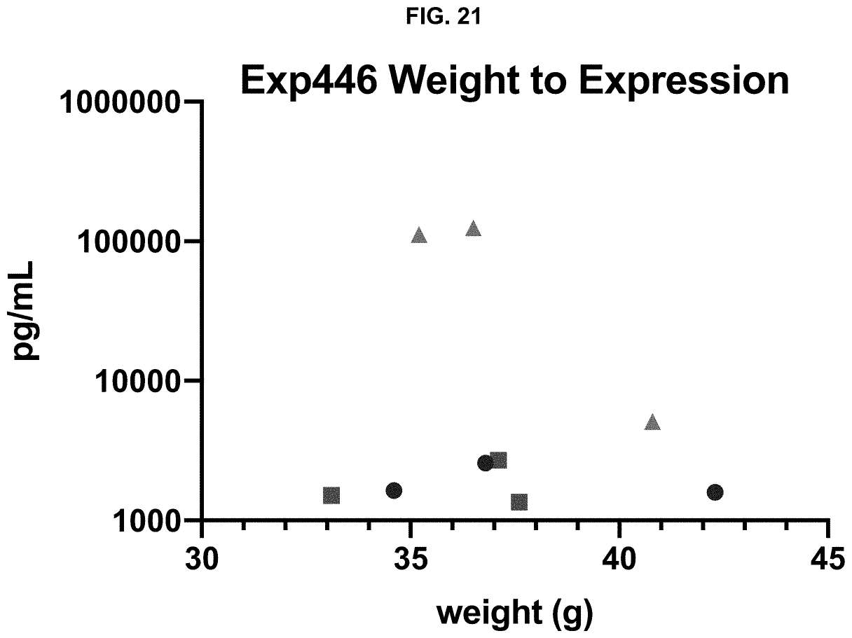

The results are shown in FIG. 20. The results indicate that 800 μg of hGLA plasmid injected one time was sufficient to produce protein expression well above the minimal therapeutic level (dotted line), starting roughly 22 days after injection, and that the group given higher dosage of hyaluronidase reached higher levels of expression faster.

Furthermore, to investigate whether the body weight of an individual mouse correlates with the hGLA expression level in the mouse, measured expression levels of hGLA were plotted against body weights of test animal in FIG. 34. As shown in the figure, no clear correlation between expression level and mice body weight was observed. These data demonstrate that long-term expression of the encoded protein using the present method does not require the subject to have obesity or have certain amount of body fat.

### Example 16

This example describes in vitro transfection of primary mouse preadipocytes cells and subsequent transplantation into mouse.

### Methods:

Two to three-week old C57BL/6 J female mice is euthanized and inguinal fat tissue is harvested and placed in HBSS buffer containing 3% (w/v) bovine serum albumin (BSA). Cells are incubated in 2 mg/mL collagenase in HBSS BSA 3% (w/v) for 30 min to digest the tissue. Cells are cultured to 80% confluence in DMEM/F12 cell culture media containing 1% Penicillin/streptomycin, 10% Fetal bovine serum, 100 μg/mL Normocin. The Preadiopcytes are then grown and plated for transfection at a concentration of 2×10⁶ cells per mL in a cell culture flask. The cells are transfected with 5 μg plasmid DNA comprising the sequence of GLA gene that encodes alpha-galactosidase A. The plasmid is complexed with 2.7 μl of Expifectamine (ThermoFisher)/μg DNA before being added to the cells. The cells are transfected overnight in an incubator. The cells are then collected, washed twice with 1× PBS, counted on a cell counter and pelleted. Each cell pellets contains about 10×10⁶ live cells and are kept on ice until implantation. Each pellet was mixed with matrigel up for a total volume of 400 μL on ice and the cell and matrigel suspension was slowly drawn into a 1 mL syringe, which is then slowly injected into three mice by tenting the subcutaneous subscapular area. Mice serum is collected every 3 days to measure the expression level of alpha-galactosidase A. The weight of mice is also monitored.

### Results:

Elevated expression of alpha-galactosidase A is detected in mouse serum and remains detectable over a course of 3 months. The mice remain healthy during the observation period.

### Example 17: Inclusion of Dexamethasone or TGF-β3 to Improve Expression of hGLA After Subcutaneous Injection

### Methods:

Groups of three mice were injected intraperitoneally or subcutaneously with 20 mg/kg body weight (high) or 2 mg/kg body weight (low) water-soluble dexamethasone along with monoclonal antibodies (mABs) to deplete B or T cells. One additional group of three mice were injected intraperitoneally with 50 or 200 ng human TGF-β3 along with mAbs to deplete B and T cells. Two groups of mice (control) were injected intraperitoneally with only depletion mAbs. 30 minutes subsequently, all mice were injected subcutaneously with 800 ug plasmid DNA encoding hGLA-1×L-hyFc in 200 uL Lactated Ringer's, along with 60U/animal hyaluronidase. Mice were bled on the 5^{th} days after injection and serum samples were collected and evaluated by ELISA.

### Results:

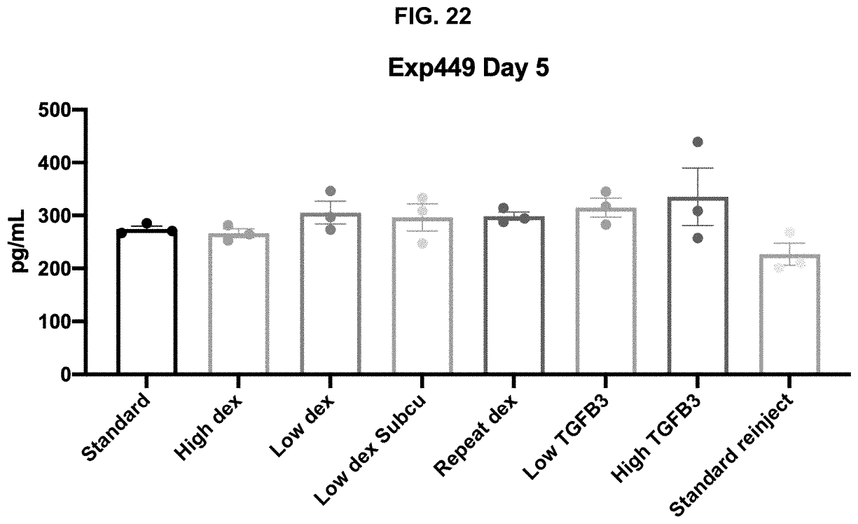

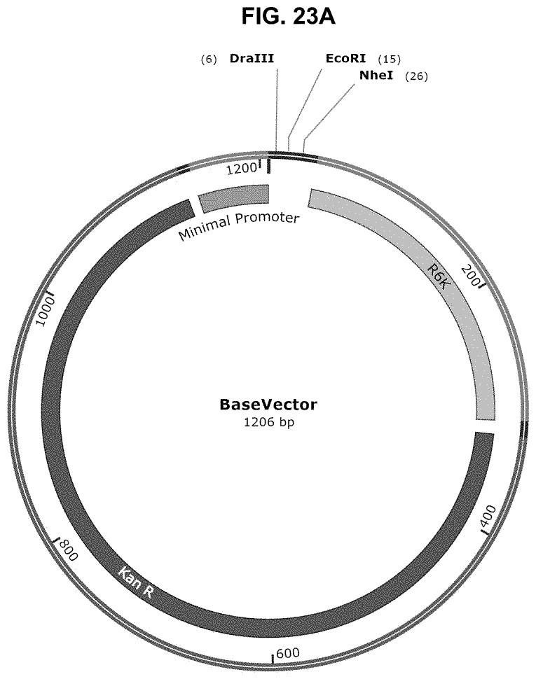

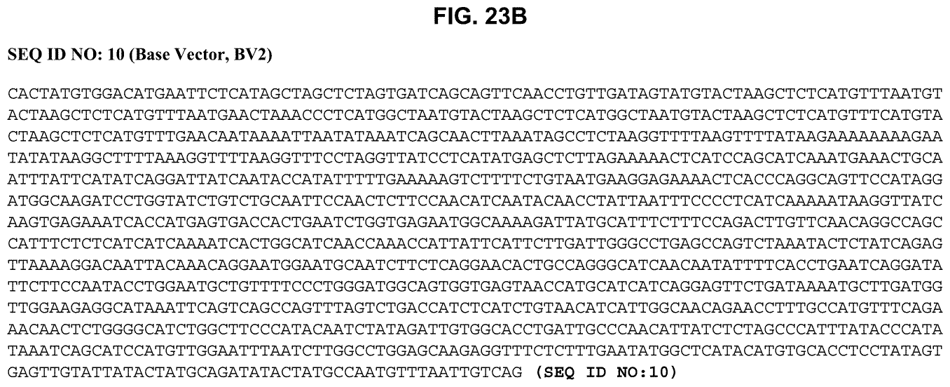

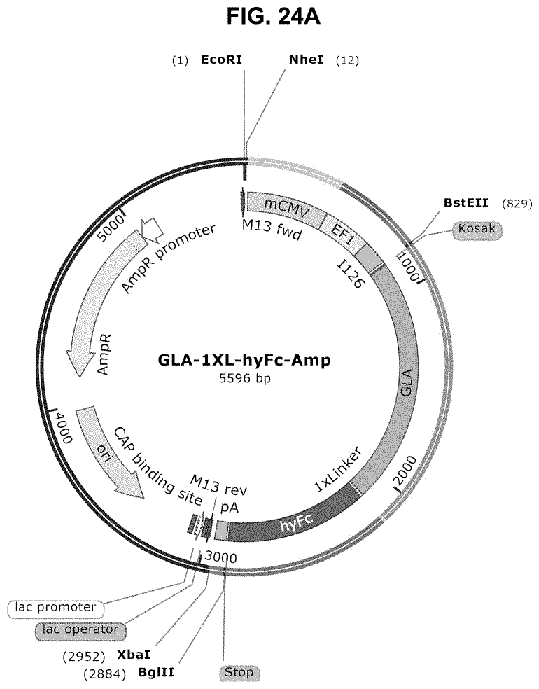

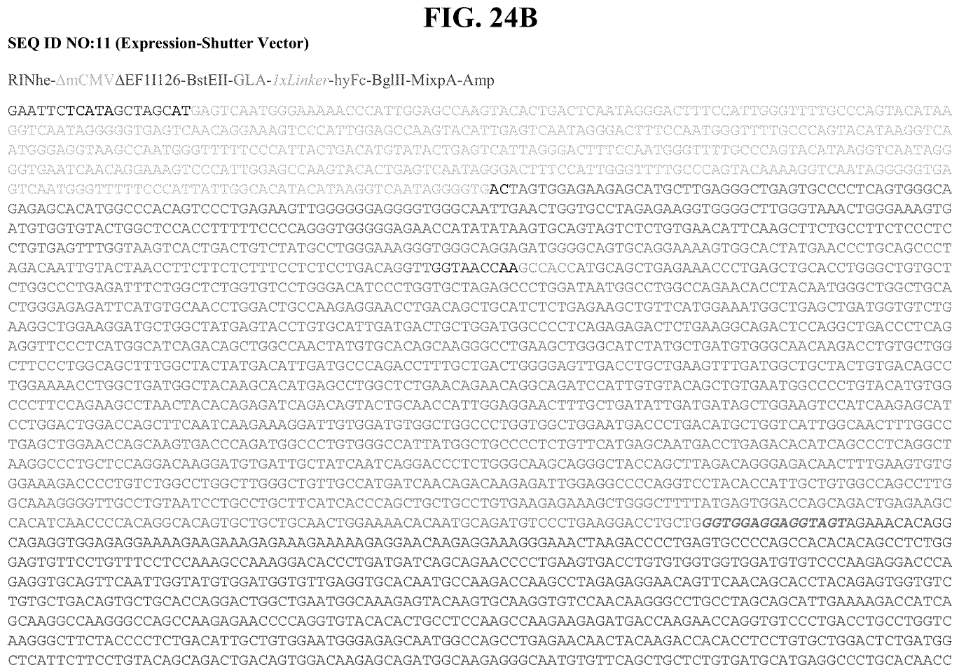

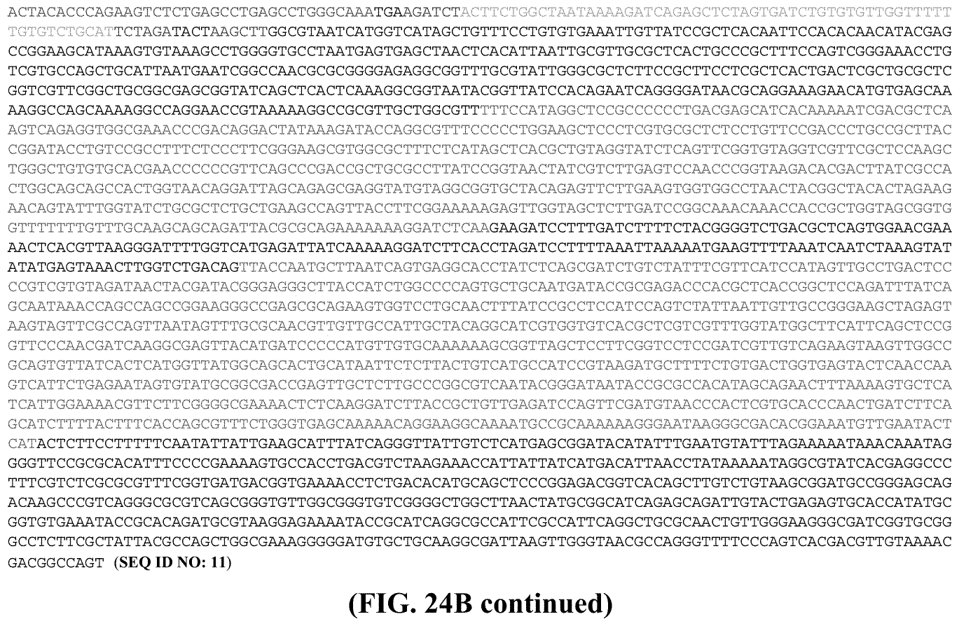

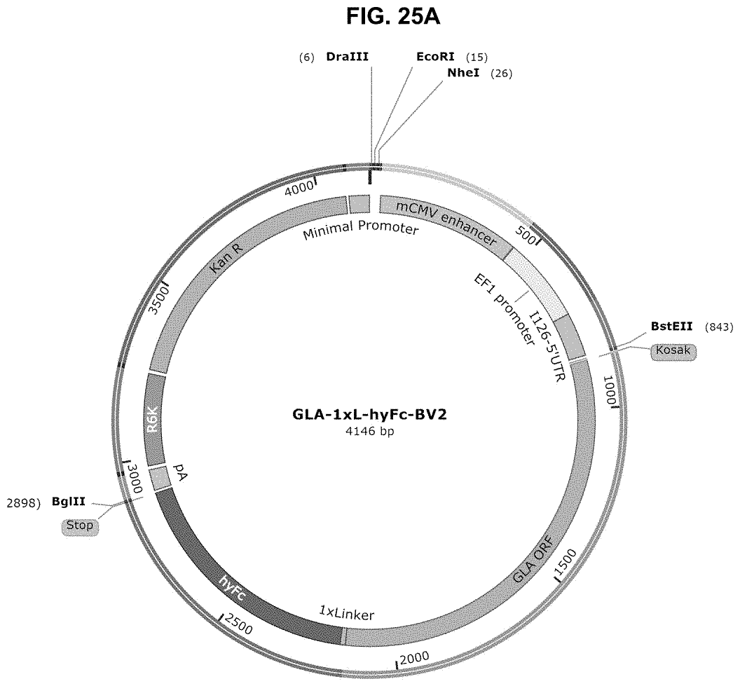

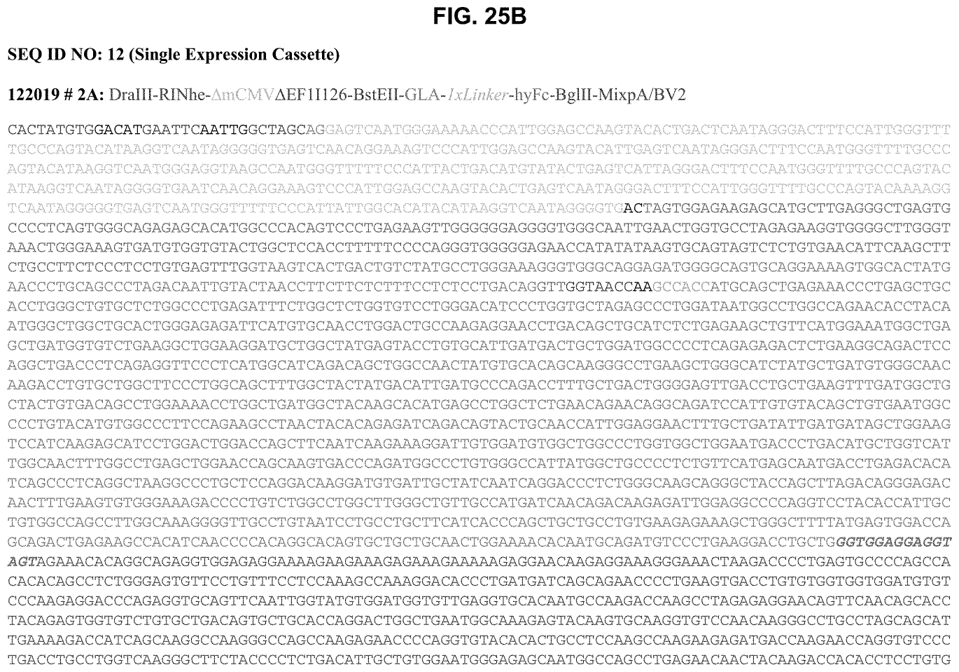

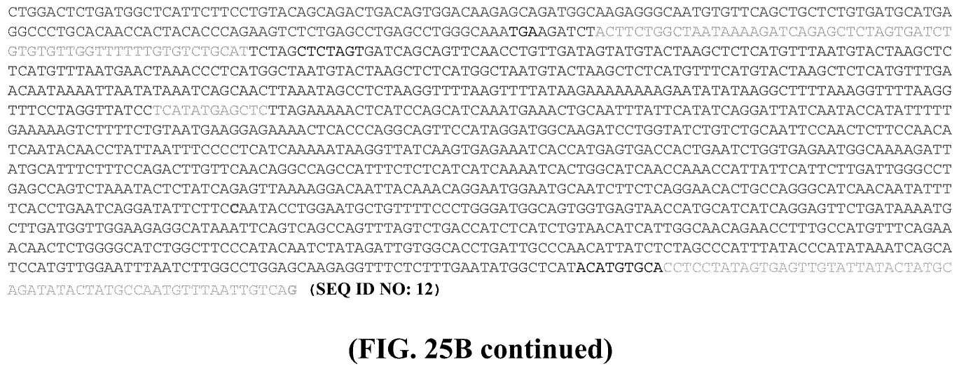

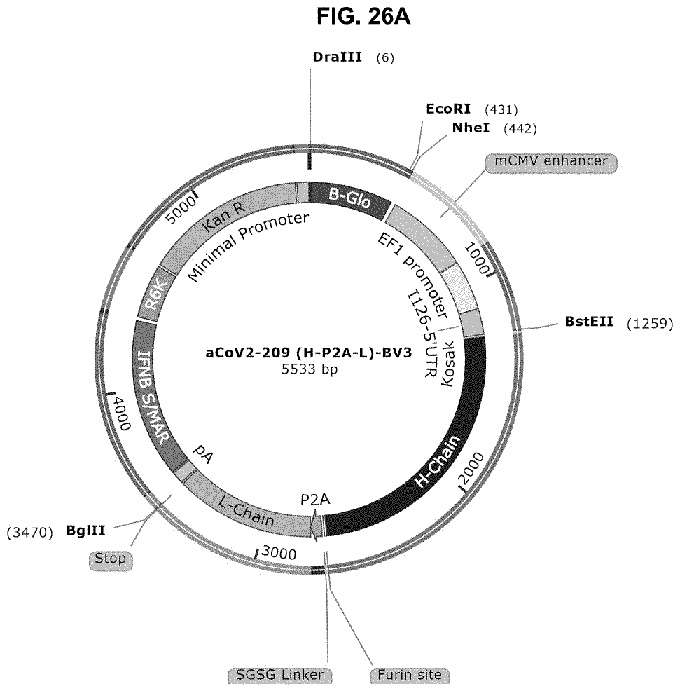

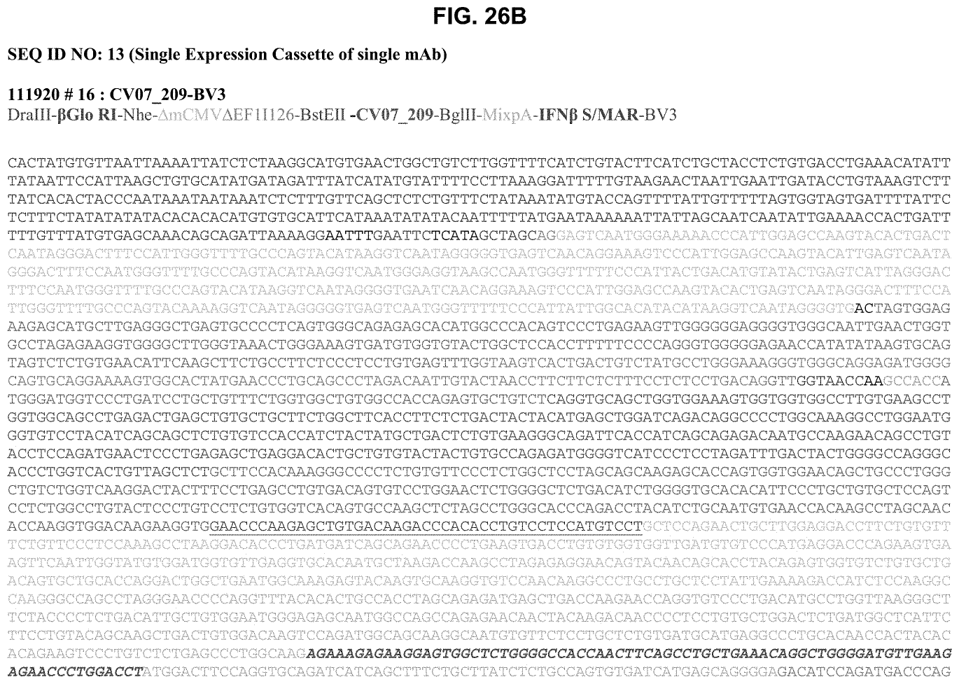

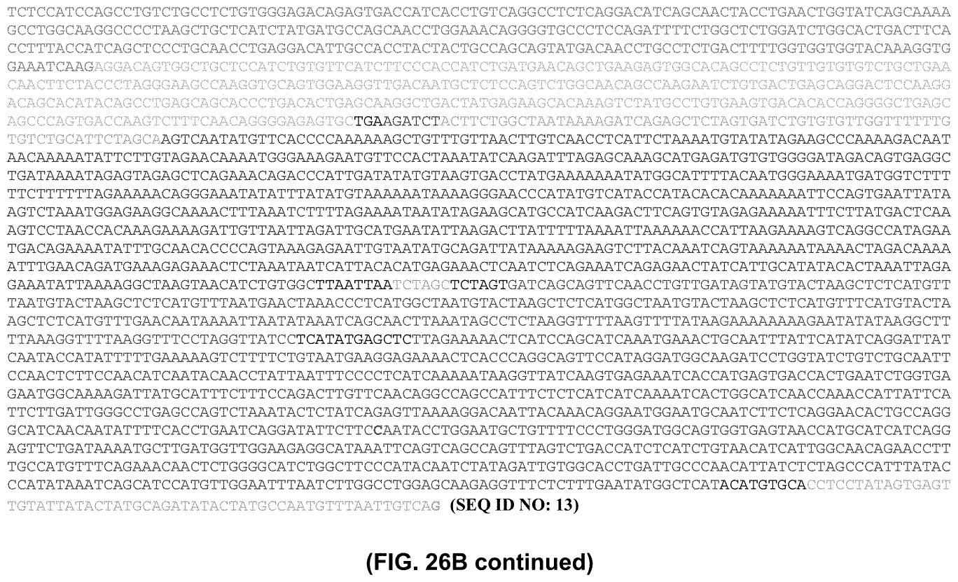

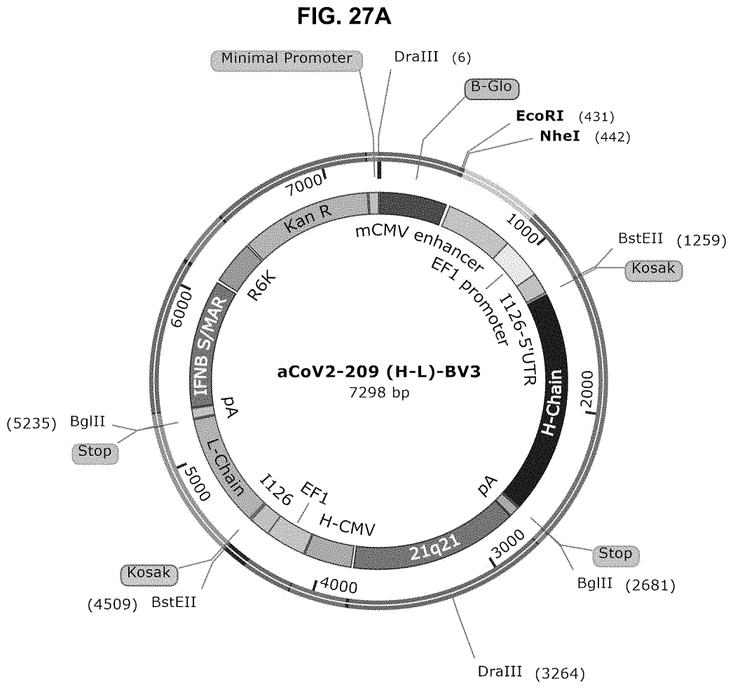

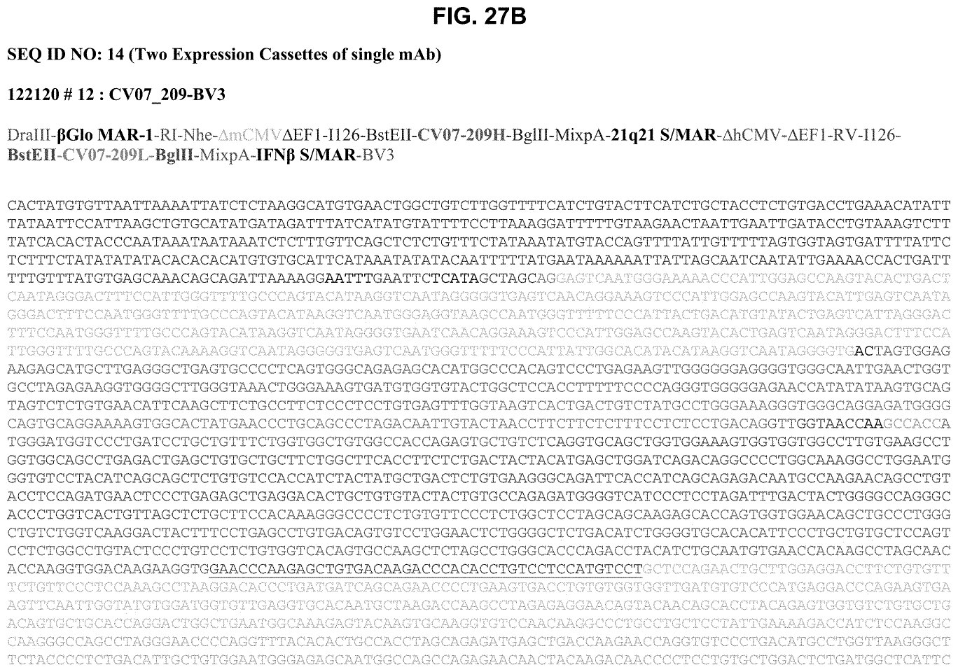

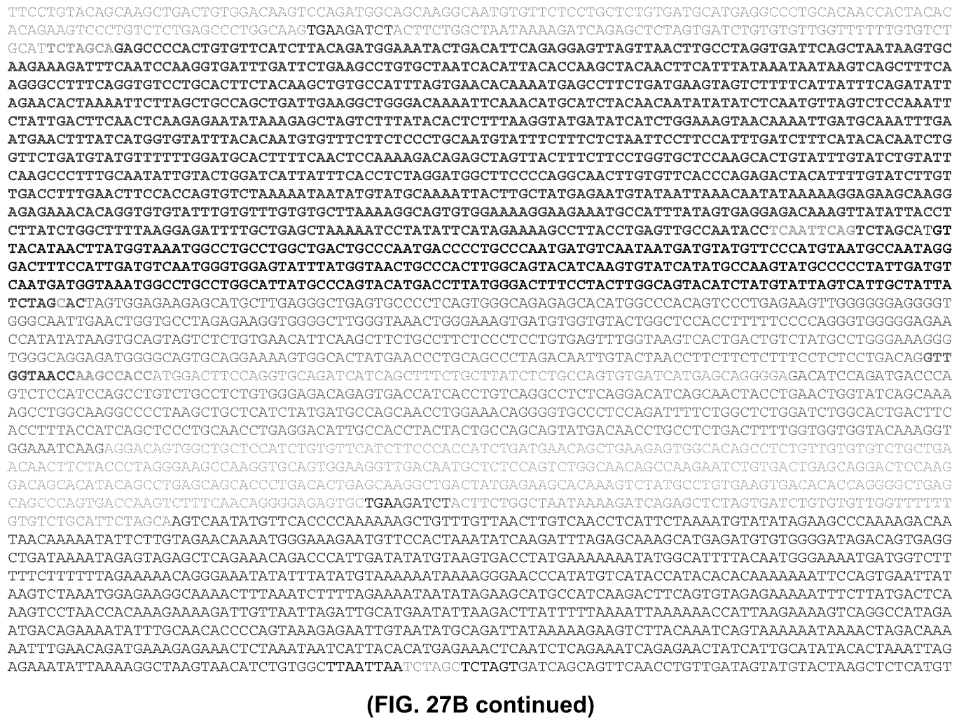

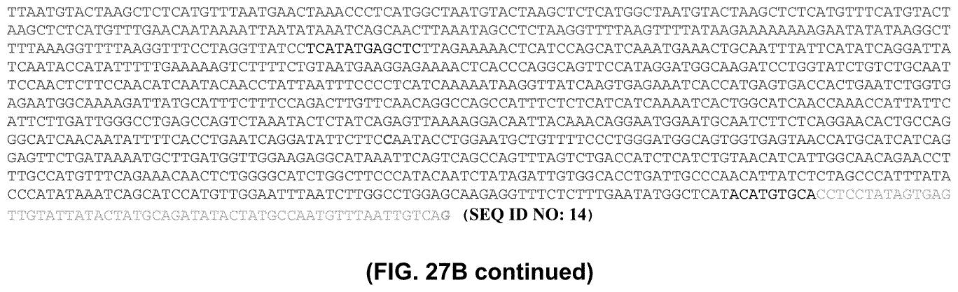

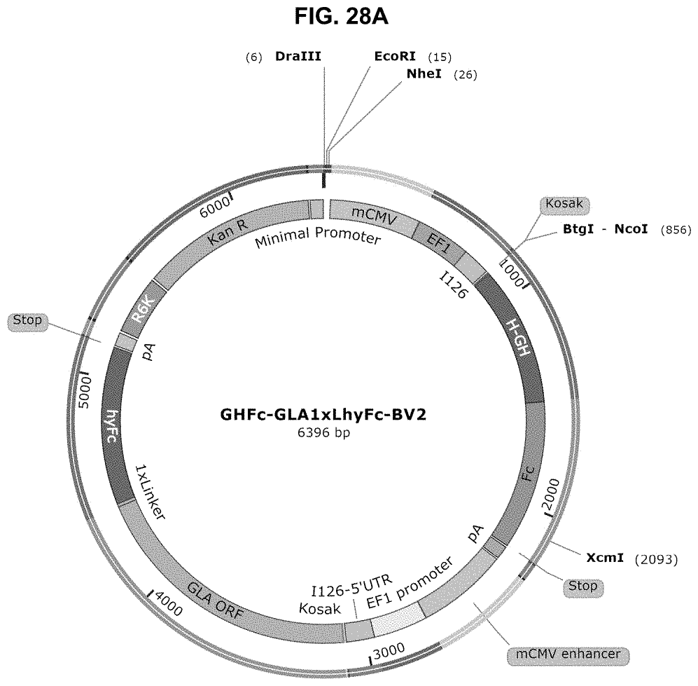

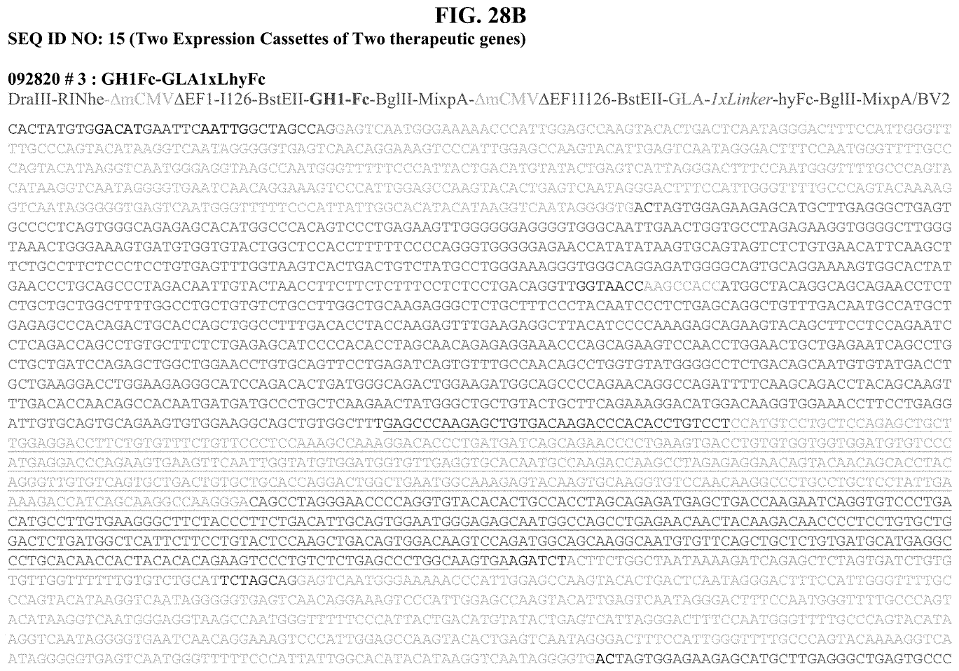

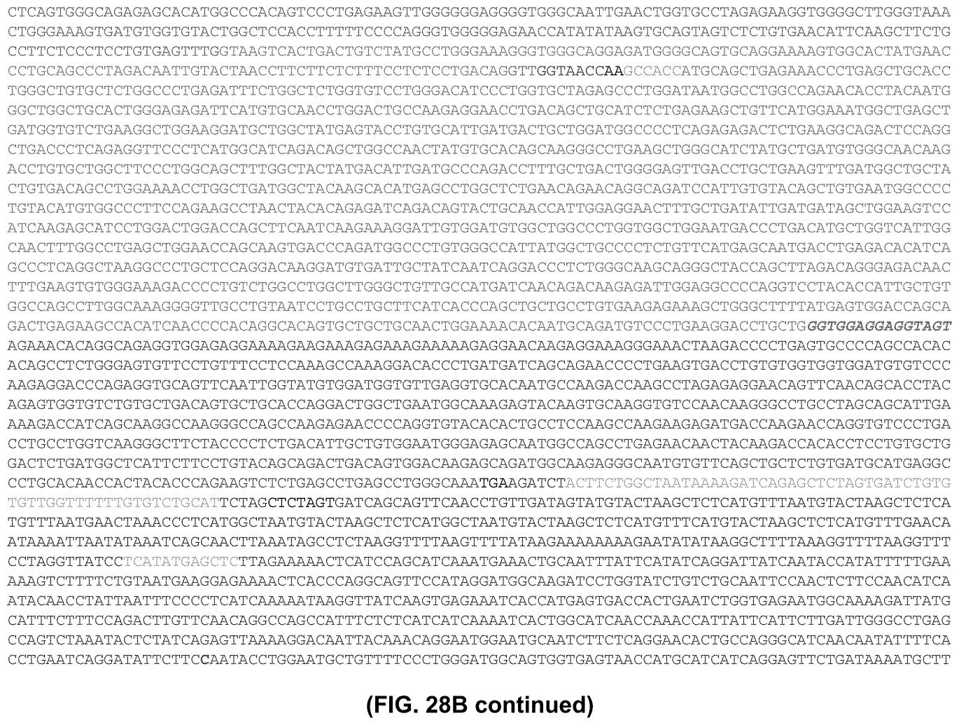

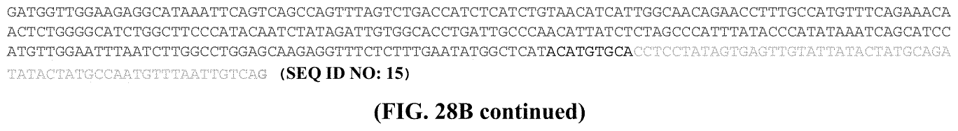

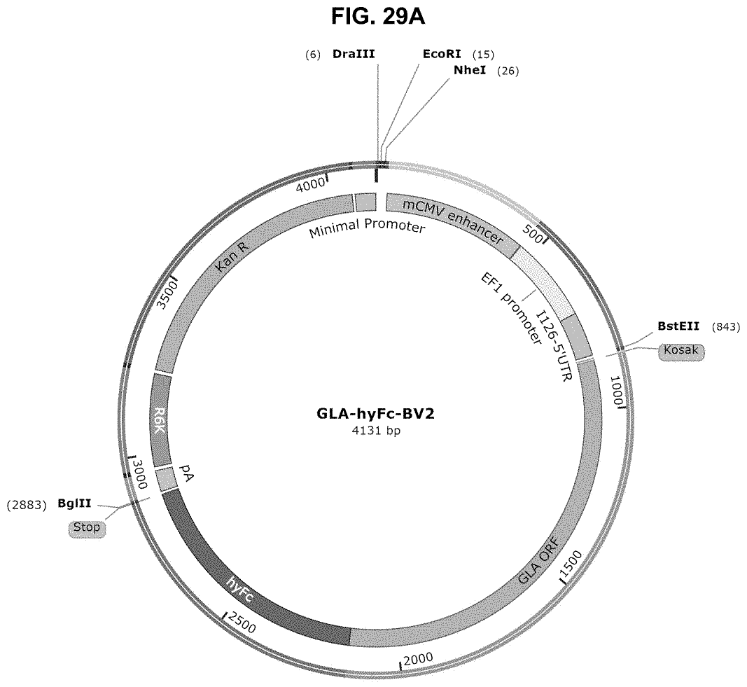

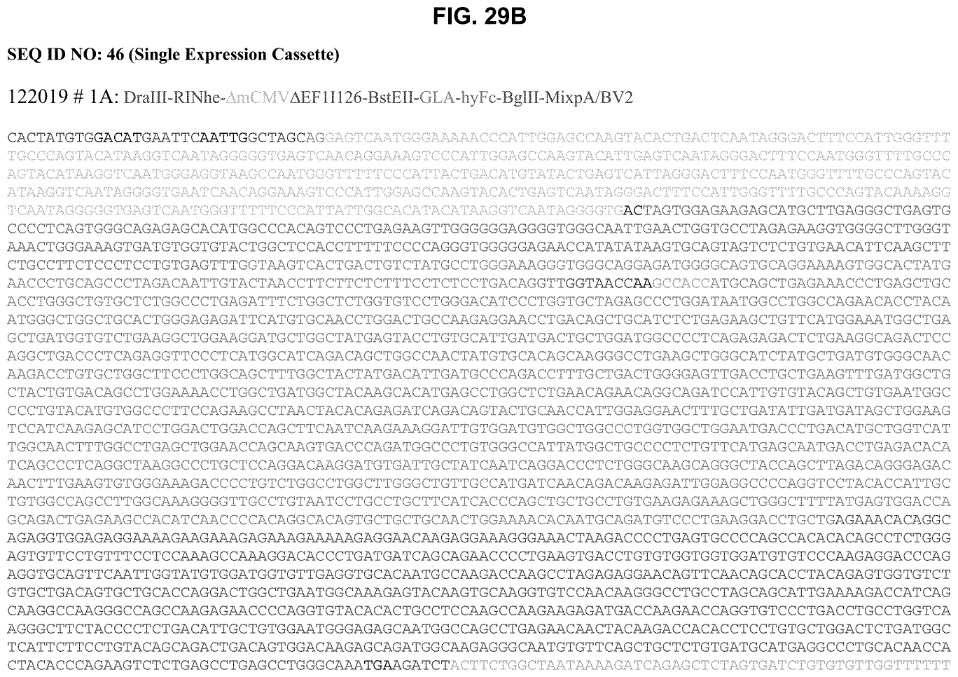

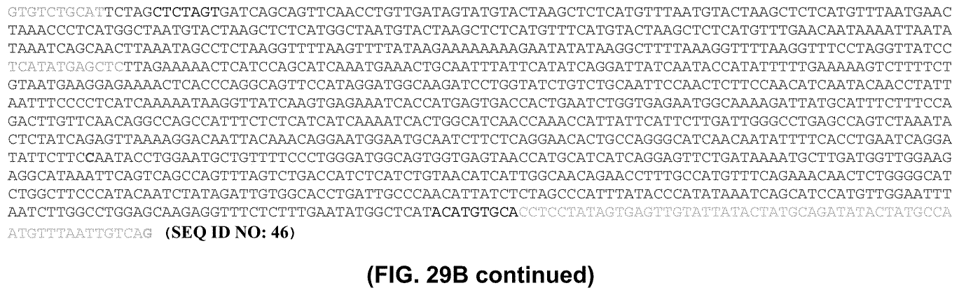

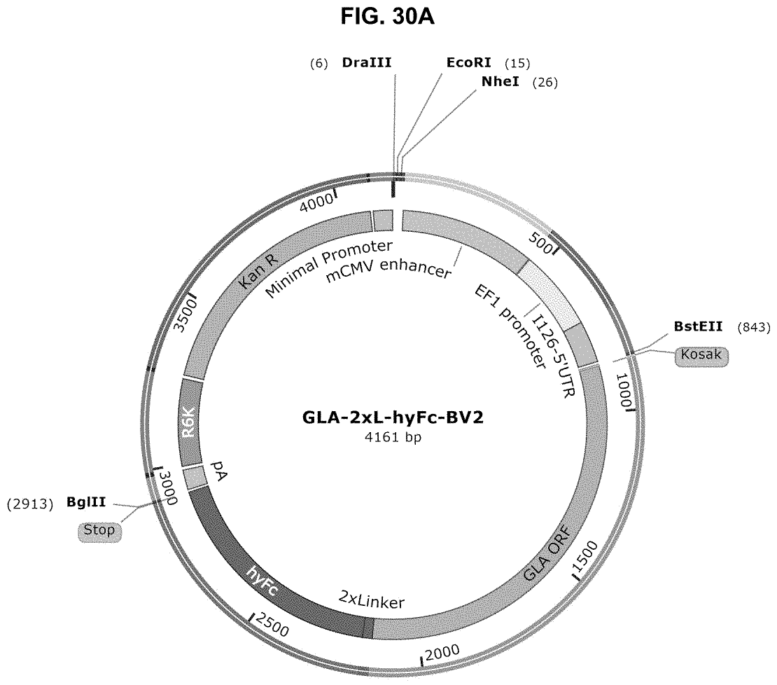

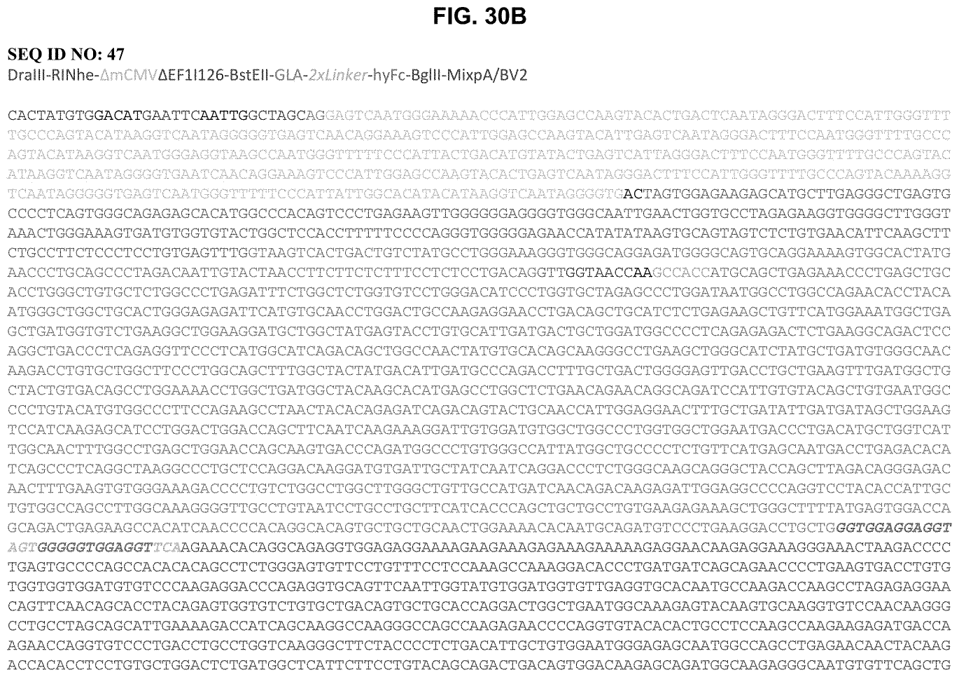

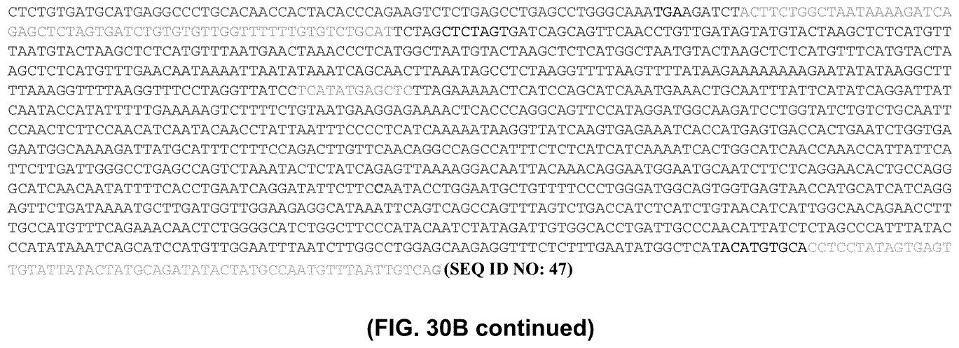

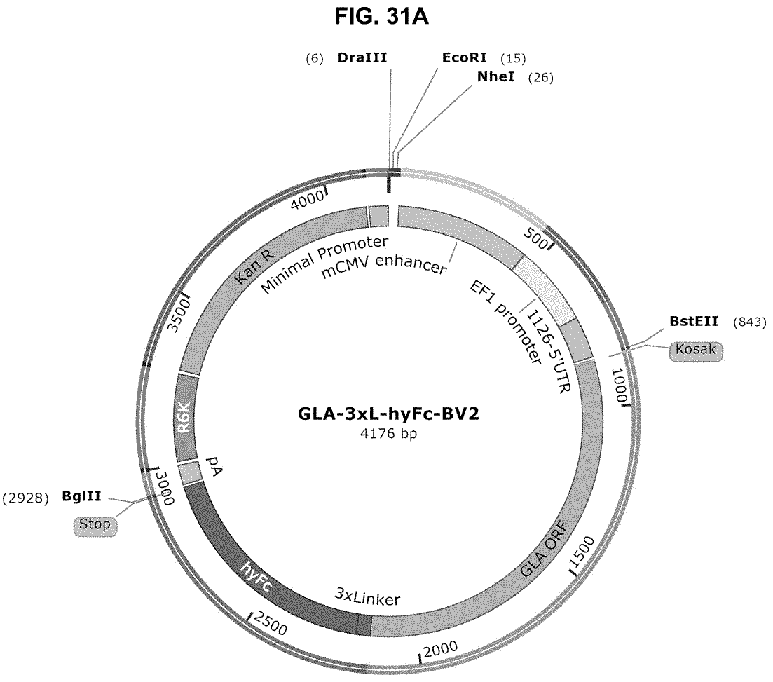

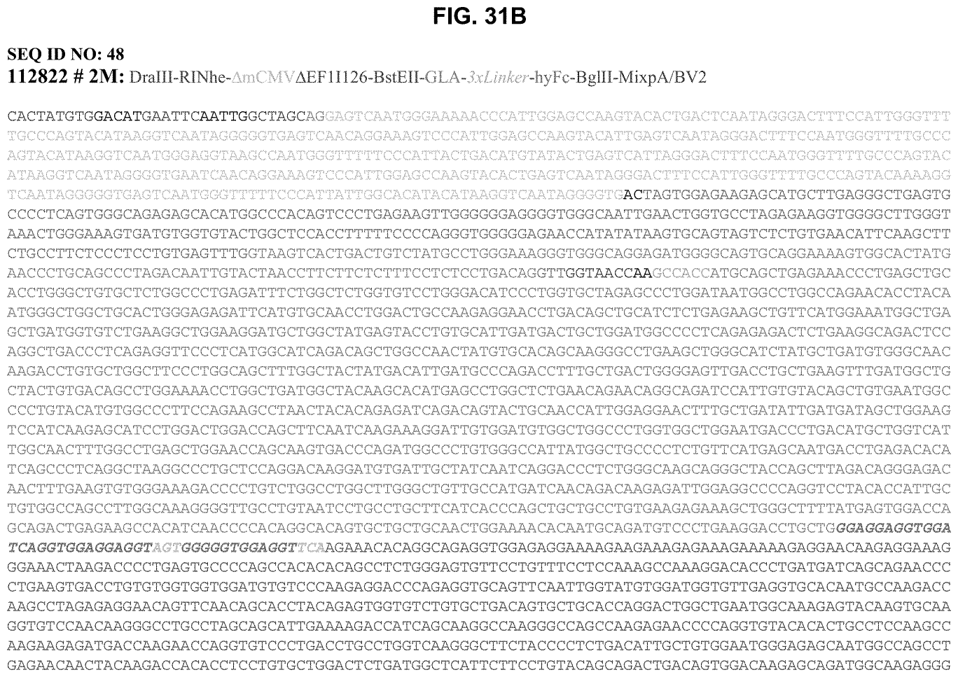

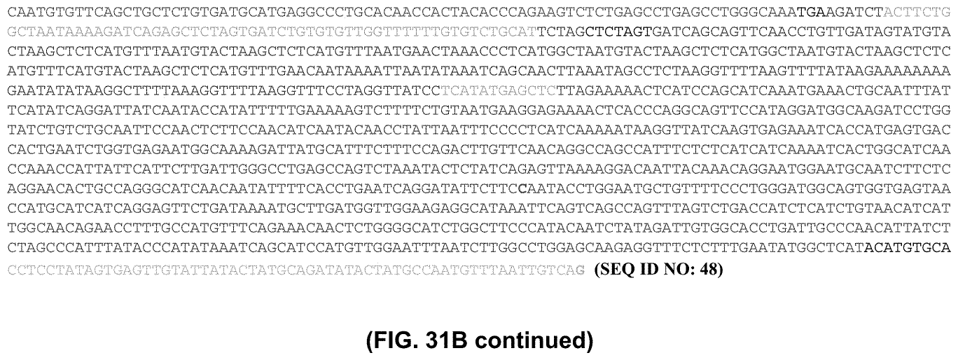

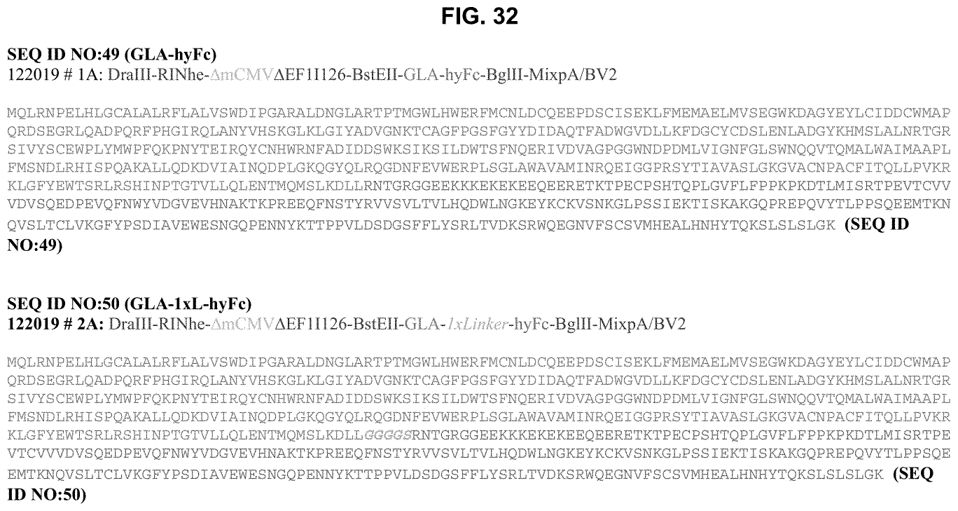

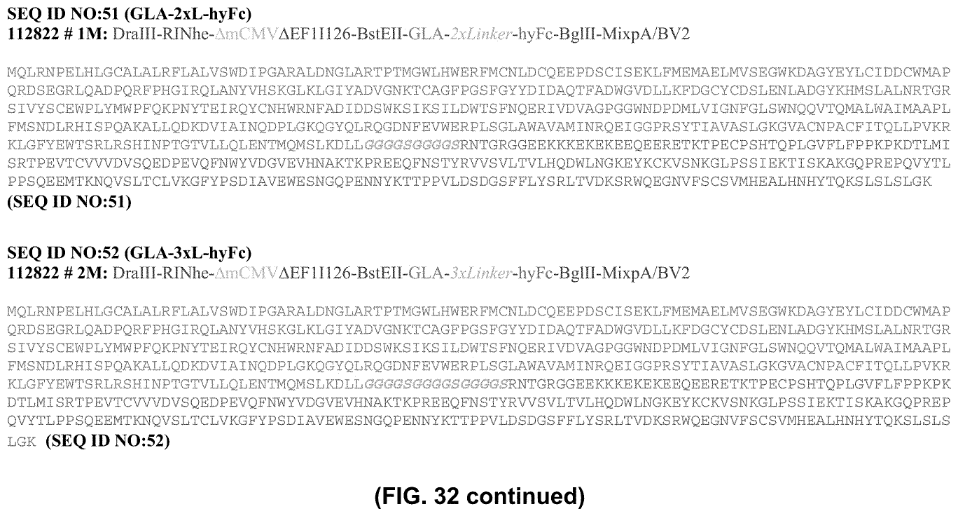

The results are shown in FIG. 22. The higher dexamethasone group performed similarly to the control group. Mice receiving intraperitoneal injection or subcutaneous injection of the low dose of dexamethasone, as well as mice receiving the higher TGF-beta3 dose performed better than the DNA and hyaluronidase alone control group.

### Example 18: Vectors Encoding Human GLA (hGLA-hyFc) Fusion Proteins

### Methods:

Groups of three mice are intraperitoneally injected with mAbs to deplete B and T Cells. The mice are subsequently injected subcutaneously once with plasmid DNAs encoding hGLA-hyFc (SEQ ID NO:49), hGLA-1×L-hyFc (SEQ ID NO:50), hGLA-2×L-hyFc (SEQ ID NO:51), and hGLA-3×L-hyFc (SEQ ID NO:52), respectively. Particularly, the 1×L linker has the sequence of GGGGS (SEQ ID NO:53), the 2×L linker has the sequence of GGGGSGGGGS (SEQ ID NO:54), and the 3×L linker has the sequence of GGGGSGGGGSGGGGS (SEQ ID NO:55). Each plasmid is mixed with 60U/ms hyaluronidase in 200 uL Lactated Ringer's before injection. Each plasmid is administered at three tested doses (400 ug/mouse, 800 ug/mouse, and 1600 ug/mouse) to three groups of mice, respectively. The following day and every week thereafter for 8 weeks, mice are bled into serum separator tubes. Resulting serum is tested for expression of hGLA protein by ELISA assay.

### Results:

For each plasmid, following the subcutaneous injection at the various doses, the tested subjects show injection-dose-dependent expressions of the encoded GLA fusion protein, with the lowest tested dose (400 ug/mouse) sufficient to produce stable expression of the encoded protein at a therapeutic level in the subject's blood. Higher dosage injection results in a higher expression level.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A method comprising: treating a subject, and/or said subject's subcutaneous pre-adipocytes and/or adipocytes, with a composition,
wherein said composition comprises, or consists essentially of, a nucleic acid sequence, or a vector containing said nucleic acid sequence, wherein said nucleic acid sequence encodes at least one protein or at least one biologically active nucleic acid molecule, and
wherein said treating comprises at least one of the following:
a) injecting said composition into one or more subcutaneous regions of said subject such that said at least one protein, or said at least one biologically active nucleic acid molecule, is detectable in a blood, serum, or plasma sample from said subject; and/or
b) injecting said composition into one or more subcutaneous regions of said subject such that a plurality of in-vivo transfected pre-adipocytes and/or adipocytes are generated in said subcutaneous region;
c) performing the following:
i) contacting a plurality said pre-adipocytes and/or adipocytes from said subject ex-vivo with said composition such that a plurality of ex-vivo transfected pre-adipocytes and/or adipocytes are generated, and
ii) injecting at least some of said plurality of ex-vivo transfected pre-adipocytes and/or adipocytes into one or more subcutaneous regions of said subject; and/or
d) implanting a solid medium carrying said composition into the one or more subcutaneous regions.

2. The method of claim 1, wherein said plurality of in-vivo transfected pre-adipocytes and/or adipocytes and/or said ex-vivo transfected pre-adipocytes and/or adipocytes are enlarged such that, on average, they contain at least 60 μg of lipid.

3. The method of claim 1, wherein said plurality of in-vivo transfected pre-adipocytes and/or adipocytes and/or said ex-vivo transfected pre-adipocytes and/or adipocytes are enlarged such that, on average, they contain at least 80 ug of lipid.

4. The method of claim 1, wherein said plurality of in-vivo transfected pre-adipocytes and/or adipocytes and/or said ex-vivo transfected pre-adipocytes and/or adipocytes are enlarged such that, on average, they have a diameter of at least 70 μm.

5. The method of claim 1, wherein said plurality of in-vivo transfected pre-adipocytes and/or adipocytes and/or said ex-vivo transfected pre-adipocytes and/or adipocytes are enlarged such that, on average, they have a diameter of at least 90 μm.

6. The method of claim 1, wherein said composition is free of any reagents that serve as aids to transfection.

7. The method of claim 1, wherein said composition comprises chloroquine, hyaluronidase, neutral liposomes, cationic liposomes, hypertonic saline, Ringer's solution, Lactated Ringer's solution, hypertonic Ringer's solution, hypotonic saline, hypotonic Ringers, hypotonic dextrose, pure water, or any combination thereof.

8. The method of claim 1, wherein said subject is overweight by BMI standards and/or is clinically obese, or wherein said subject is not overweight by BMI standards and/or is clinically obese.

9. The method of claim 1, wherein said injecting in a), b), or c) ii) is into a fat pad of said subject.

10. The method of claim 1, wherein said at least one protein comprises an antibody variable region.

11. The method of claim 10, wherein said antibody variable region is a light chain or heavy chain variable region.

12. The method of claim 1, wherein said at least one protein comprises a least a portion of an antibody light chain and at least a portion of an antibody heavy chain.

13. The method of claim 1, wherein said at least one protein comprises an antibody light chain and an antibody heavy chain, wherein said light and heavy chains together form a monoclonal antibody or antigen binding fragment thereof.

14. The method of claim 13, wherein said monoclonal antibody, or antigen binding portion thereof, are specific for SARS-CoV-2 or influenza.

15. The method of claim 13, wherein said monoclonal antibody, or antigen binding portion thereof, is selected from those listed in Table 4.

16. The method of claim 13, wherein said subject is infected with a pathogen, and wherein said monoclonal antibody, or antigen binding portion thereof, is expressed in said blood, serum, or plasma at pathogen-neutralizing levels.

17. The method of claim 1, further comprising, prior to said injecting in a), b), or c) ii), administering a solution comprising cationic liposomes and/or neutral liposomes to said subject.

18. The method of claim 17, wherein said solution comprises dexamethasone and/or dexamethasone palmitate.

19. The method of claim 17, wherein said administering said cationic and/or neutral liposomes is performed intravenously or subcutaneously.

20. The method of claim 1, wherein said injecting in a), b), or c) ii) is performed at a plurality of sites in said subject.

21. The method of claim 1, wherein said injecting in a) and/or b) causes said subject to receive between 1 and 60 micrograms, or between 0.00001 and 1.0 micrograms, per microliter of said composition of said nucleic acid sequence, or said vector containing said nucleic acid sequence.

22. The method of claim 1, wherein said vector comprises a non-viral vector, optionally wherein the vector further comprises one or more DNA expression cassettes; optionally wherein said vector is a plasmid.

23. The method of claim 1, wherein said nucleic acid sequence comprises DNA.

24. The method of claim 1, wherein said nucleic acid comprises mRNA, and wherein said mRNA is optionally capped and composed of at least some modified bases that reduce immunogenicity.

25. The method of claim 1, wherein said subject is a human.

26. The method of claim 1, wherein said at least one biologically active nucleic acid molecule comprises a sequence selected from: an siRNA, shRNA sequence, a miRNA sequence, an antisense sequence, a CRISPR single guide RNA sequence (sgRNA), piRNA, snoRNA, tsRNA, and srRNA.

27. The method of claim 1, wherein said nucleic acid sequence or vector are CPG-free or CPG-reduced.

28. The method of claim 1, wherein said at least one protein is selected from the group consisting of:
human growth hormone, G-CSF protein, erythropoietin, Etanercept, Bevacizumab, Rituximab, Adalimumab, Infliximab, Trastuzumab, Insulin, Insulin glargine, Epoetin alfa, Pegfilgrastim, Ranibizumab, Darbepoetin alfa, Interferon beta-1a, Interferon beta-1a (Rebif), Insulin aspart, Rhu insulin, Octocog alfa, Insulin lispro, Cetuximab, Peginterferon alfa-2a, Interferon beta-1b, Eptacog alfa, Insulin aspart, OnabotulinumtoxinA, Epoetin beta, Rec antihemophilic factor, Filgrastin, Insulin detemir, Natalizumab, Insulin (humulin), ACE2, Palivizumab, and a-galactosidase A (GLA).

29. The method of claim 1, wherein said at least one protein and/or said at least one biologically active nucleic acid, is expressed in said subject at a level of at least 50 μg/ml or at least 100 ng/ml, in blood, serum, or plasma, for at least 10 days, or at least 25 days.

30. The method of claim 1, wherein said at least one protein and/or said at least one biologically active nucleic acid, is expressed in said subject at a level of at least 1000 ng/ml, in blood, serum, or plasma, for at least 10 days, or at least 25 days or at least 114 days after subcutaneous injection.

31. The method of claim 29, wherein said expression level is maintained in said subject for at least one month without any further treatment.

32. The method of claim 17, wherein said cationic and neutral lipids are selected from the group consisting of: DOTAP (1,2-dioleoyl-3-trimethylammonium-propane); distearoyl phosphatidyl choline (DSPC); hydrogenated or non-hydrogenated soya phosphatidylcholine (HSPC); distearoylphosphatidylethanolamine (DSPE); egg phosphatidylcholine (EPC); 1,2-Distearoyl-sn-glycero-3-phospho-rac-glycerol (DSPG); dimyristoyl phosphatidylcholine (DMPC); 1,2-Dimyristoyl-sn-glycero-3-phosphoglycerol (DMPG); 1,2-Dipalmitoyl-sn-glycero-3-phosphate (DPPA); trimethylammonium propane lipids; DOTIM (1-[2-9(2)-octadecenoylloxy)ethyl]-2-(8(2)-heptadecenyl)-3-(2-hydroxyethyl) midizolinium chloride) lipids; and mixtures of two or more thereof.

33. The method of claim 1, wherein said one or more non-viral expression vectors comprise plasmids, wherein said plasmids are not attached to, or encapsulated in, any delivery agent.

34. The method of claim 1, wherein said at least one protein comprises at least one anti-SARS-CoV-2 monoclonal antibody, or antigen-binding portion thereof, and is optionally selected from the group consisting of: VIR-7831; LY-CoV1404; LY3853113; Zost 2355K; CV07-209K; C121L; Zost 2504L; CV38-183L; COVA215K; RBD215; CV07-250L; C144L; COVA118L; C135K; and B38.

35. The method of claim 34, wherein said at least one anti-SARS-CoV-2 antibody, or antigen-binding portion thereof, comprises at least two anti-SARS-CoV-2 antibodies, and/or antigen-binding portions thereof, which are expressed in said subject at an expression level sufficient to reduce: i) the SARS-CoV-2 viral load in said subject, and/or ii) at least one symptom in said subject caused by said SARS-CoV-2 infection.

36. The method of claim 34, wherein said at least one anti-SARS-CoV-2 antibody, or antigen-binding portion thereof, comprises at least four, or at least eight, or at least 11, anti-SARS-CoV-2 antibodies and/or antigen-binding portions thereof.

37. The method of claim 1, wherein, prior to said injecting, said subject is treated with dexamethasone fatty acid ester.

38. The method of claim 1, wherein said composition further comprise a physiologically tolerable buffer or intravenous solution.

39. The method of claim 1, wherein prior to said injecting, the method further comprises administering a solution to said subject comprising liposomes comprising said polycationic structures, wherein said liposomes further comprising one or more macrophage targeting moieties selected from the group consisting of: mannose moieties, maleimide moieties, a folate receptor ligand, folate, folate receptor antibody or fragment thereof, formyl peptide receptor ligands, N-formyl-Met-Leu-Phe, tetrapeptide Thr-Lys-Pro-Arg, galactose, lactobionic acid, a lipid bi-layer integrating peptide and/or a target peptide.

40. The method of claim 1, wherein 0.05-60 mg/mL of said vectors are present in said composition.

41. The method of claim 1, wherein in d) the implanting a is performed via an incision surgery.

42. The method of claim 1, further comprising, prior to said injecting in a), b), or c) ii) or said implanting in d), administering a composition comprising TGF-β3 to said subject, optionally wherein the composition comprising TGF-β3 is administered intravenously.

43. The method of claim 1, wherein prior to said injecting, said subject is treated with TGF-133.

44. A system comprising:
a) a plurality of transfected and enlarged adipocytes or pre-adipocytes, wherein each of said plurality of transfected and enlarged adipocytes or pre-adipocytes comprises an exogenous nucleic acid sequence, or a vector containing said nucleic acid sequence, wherein said nucleic acid sequence encodes at least one protein or at least one biologically active nucleic acid molecule, and
b) a first container, wherein said plurality of transfected and enlarged adipocytes or pre-adipocytes are present in said first container.

45. The system of claim 44, wherein said first container comprises a syringe configured for injecting said plurality of transfected and enlarged adipocytes or pre-adipocytes into a subject subcutaneously.

46. The system of claim 44, further comprising: c) a solution comprising at least one of the following: i) cationic liposomes, ii) neutral liposomes, iii) dexamethasone, and iv) dexamethasone palmitate.

47. The system of claim 44, wherein the plurality transfected and enlarged adipocytes or pre-adipocytes contain, on average, at least 60 pg of lipid.

48. The system of claim 44, wherein the plurality transfected and enlarged adipocytes or pre-adipocytes contain, on average, at least 80 pg of lipid.

49. The system of claim 44, wherein said transfected and enlarged adipocytes or pre-adipocytes have, on average, a diameter of at least 70 μm.

50. The system of claim 44, wherein said transfected and enlarged adipocytes or pre-adipocytes have, on average, a diameter of at least 90 μm.

51. The system of claim 44, wherein said transfected and enlarged adipocytes or pre-adipocytes are derived from one or more subcutaneous regions of a subject.
